(19) United States
(12) Patent Application Publication
(10) Pub. No.: US 2026/0159515 A1
(43) Pub. Date: Jun. 11, 2026

(54) BENZAMIDE AMINE ARYL ETHER ANALOGS AS INHIBITORS OF THE MENIN-MLL INTERACTION

(71) Applicant: Syndax Pharmaceuticals, Inc., New York, NY (US)

(72) Inventors: Gerard M. MCGEEHAN, Newtown Square, PA (US); William H. MILLER, Collegeville, PA (US); Swapan K. SAMANTA, Hyderabad (IN); Virsinha REDDY, Hyderabad (IN); Sangmeshwar P. SAWARAGVE, Hyderabad (IN); Singam Naveen KUMAR, Hyderabad (IN); Pradeep JADHAVAR, Hyderabad (IN)

(21) Appl. No.: 19/410,434

(22) Filed: Dec. 5, 2025

Related U.S. Application Data
(60) Provisional application No. 63/729,167, filed on Dec. 6, 2024.
(60) Provisional application No. 63/763,167, filed on Feb. 25, 2025.
(60) Provisional application No. 63/914,807, filed on Nov. 10, 2025.

(51) Int. Cl. C07D 471/04; A61K 31/501; A61K 31/506; A61K 31/53; A61K 31/5377; A61K 31/5386; A61P 35/02; C07D 401/14; C07D 403/14; C07D 471/14; C07D 519/00

(52) U.S. Cl. CPC C07D 471/04; A61K 31/501; A61K 31/506; A61K 31/53; A61K 31/5377; A61K 31/5386; A61P 35/02; C07D 401/14; C07D 403/14; C07D 471/14; C07D 519/00

(57) ABSTRACT

The present disclosure is directed to benzamide amine aryl ether analogs of Formula I, or a stereoisomer thereof, or a pharmaceutically acceptable salt thereof, as inhibitors of the interaction of menin with MLL and MLL fusion proteins, pharmaceutical compositions containing the same, and their use in the treatment of cancer and other diseases mediated by the menin-MLL interaction.

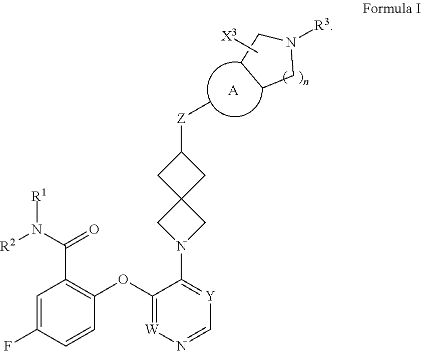

### RELATED APPLICATIONS

This application claims priority to U.S. Provisional Application No. 63/729,167, filed Dec. 6, 2024, U.S. Provisional Application No. 63/763,167, filed Feb. 25, 2025, and U.S. Provisional Application No. 63/914,807, filed Nov. 10, 2025, the entire contents of each of which is incorporated herein by reference.

### BACKGROUND

The mixed-lineage leukemia (MLL), also known as KMT2A, gene encodes a protein that is a histone methyltransferase that is mutated in clinically and biologically distinctive subsets of acute leukemia. Rearranged mixed lineage leukemia (MLL-r) involves recurrent translocations of the 11q23 chromosome locus which lead to an aggressive form of acute leukemia with limited therapeutic options. These translocations target the MLL gene creating an oncogenic fusion protein comprising the amino-terminus of MLL fused in frame with more than 60 different fusion protein partners. Menin, a ubiquitously expressed, nuclear protein encoded by the multiple endocrine neoplasia type 1 (MEN1) tumor suppressor gene, has a high affinity binding interaction with MLL fusion proteins and is an essential co-factor of oncogenic MLL-r fusion proteins. Disruption of this interaction leads to selective growth inhibition and apoptosis of MLL-r leukemia cells both in vitro and in vivo.

The interaction between menin and MLL or MLL fusion proteins is an attractive target for therapeutic intervention, and there is a need for novel agents that inhibit the menin-MLL interaction for the treatment of various diseases and conditions, including leukemia, other cancers, and diabetes.

### SUMMARY

In one aspect, the present disclosure is directed to a compound of Formula I′,

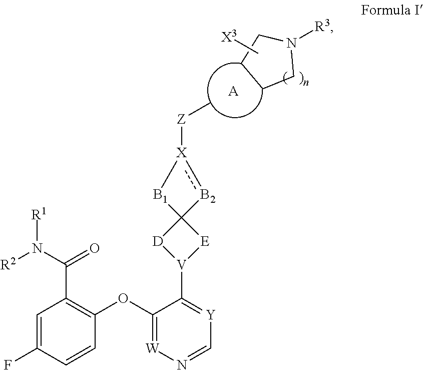

a stereoisomer, or a pharmaceutically acceptable salt thereof, wherein:
[symbol] denotes a single bond or a double bond, as valency permits;
B₁, B₂, D, and E are each independently selected from —C(R^{A1})(R^{A2})—, —C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)— wherein no more than one of B₁, B₂, D, and E is —C(R^{A1})(R^{A2})—O—, —C(R^{A1})(R^{A2})NR^{A3}—, —C(R^{A1})(R^{A2})—, C(═O)—, —C(═O)—, or —N═C(NH₂)—;
V is N or CR^{W}, wherein R^{W} is H, halo, CN, OH, C₁-C₄ alkyl, C₁-C₄ alkoxy, amino, C₁-C₄ alkyl amino, or C₂-C₈ dialkylamino;
X is N or CR^{X}, wherein R^{X} is absent, H, halo, CN, OH, C₁-C₄ alkyl, C₁-C₄ alkoxy, amino, C₁-C₄ alkyl amino, or C₂-C₈ dialkylamino;
each R^{A1} is independently selected from absent, H, halo, C₁-C₄ alkyl, C₁-C₄ alkoxy, C₁-C₄ haloalkyl, C₁-C₄ haloalkoxy, amino, C₁-C₄ alkylamino, C₂-C₈ dialkylamino, CN, NO₂, and OH;
each R^{A2} is independently selected from H, halo, C₁-C₄ alkyl, C₁-C₄ alkoxy, C₁-C₄ haloalkyl, C₁-C₄ haloalkoxy, amino, C₁-C₄ alkylamino, C₂-C₈ dialkylamino, CN, NO₂, and OH;
each R^{A3} is independently selected from H, C₁-C₄ alkyl, C₁-C₄ alkoxy, C₁-C₄ haloalkyl, C(O)R^{z}, and C(O)OR^{z}, wherein said C₁-C₄ alkyl is optionally substituted by phenyl, C₁-C₄ alkoxy, C₁-C₄ haloalkoxy, CN, NO₂, or OH;
R^{z} is H, C₁-C₄ alkyl, or phenyl;
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Z is O, CH₂, or NH;
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′})
each R^{4b} is independently H, S(═O)₂R^{4b′}, C(O)OR^{4b′}, C(O)R^{4b′}, C(O)NR^{4b′}, R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.

In one aspect, the present disclosure is directed to a compound of Formula I,

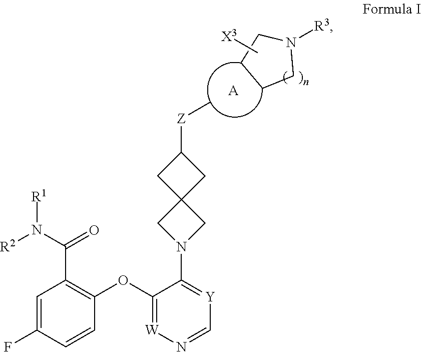

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1bs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Z is O, CH₂, or NH,
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.

In one aspect, the present disclosure is directed to a compound of Formula I-a,

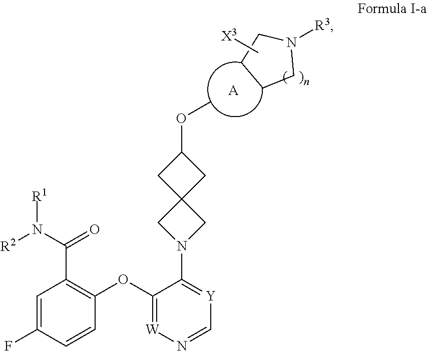

a stereoisomer, or a pharmaceutically acceptable salt thereof, wherein:
W is N or CH:
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(R^{3a′})(R^{3b′}), N(R^{3a′})(R^{3b′}), or S(═O)₂R^{3a′};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1es};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4′}), N(R^{4′})C(O)O(R′), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.

In one aspect, the present disclosure is directed to a compound of Formula I-b,

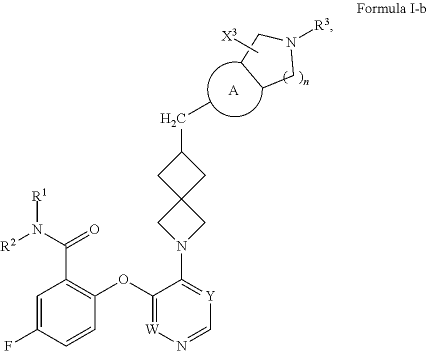

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{4a′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.

In one aspect, the present disclosure is directed to a compound of Formula I-c,

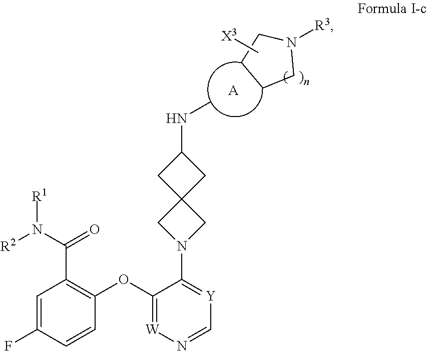

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{3a′};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});

Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R*), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.

In one aspect, the present application relates to a pharmaceutical composition comprising a compound of the application, or a pharmaceutically acceptable salt, and a pharmaceutically acceptable carrier.

In one aspect, the present application relates to a pharmaceutical composition comprising a therapeutically effective amount of a compound of the application, or a pharmaceutically acceptable salt, and a pharmaceutically acceptable carrier.

In one aspect, the present application relates to a pharmaceutical composition comprising a compound of the application, and a pharmaceutically acceptable carrier.

In one aspect, the present application relates to a pharmaceutical composition comprising a therapeutically effective amount of a compound of the application, and a pharmaceutically acceptable carrier.

The present disclosure further provides a use of a compound of Formula I′, I, I-a, I-b, or I-c, or a stereoisomer, or a pharmaceutically acceptable salt thereof in a method of treating cancer in a patient in need thereof.

The present disclosure further provides a compound of Formula I′, I, I-a, I-b, or I-c, or a stereoisomer, or a pharmaceutically acceptable salt thereof for use in a method of treating cancer in a patient in need thereof.

The present disclosure further provides a compound of Formula I′, I, I-a, I-b, or I-c, or a stereoisomer, or a pharmaceutically acceptable salt thereof for use in the manufacture of a medicament for the treatment of cancer in a patient in need thereof.

The present disclosure further provides a method of inhibiting the interaction between menin and MLL comprising contacting the menin and MLL with an effective amount of a compound of Formula I′, I, I-a, I-b, or I-c, or a stereoisomer, or a pharmaceutically acceptable salt thereof.

The present disclosure further provides a method of treating cancer in a patient comprising administering to the patient a therapeutically effective amount of a compound of Formula I′, I, I-a, I-b, or I-c, a stereoisomer, or a pharmaceutically acceptable salt thereof.

The present disclosure further provides a method of inhibiting the interaction between menin and MLL comprising contacting the menin and MLL with a compound of Formula I′, I, I-a, I-b, or I-c, a stereoisomer, or a pharmaceutically acceptable salt thereof.

The present disclosure further provides a method of treating cancer in a patient comprising administering to the patient an effective amount of a compound of Formula I′, I, I-a, I-b, or I-c, a stereoisomer, or a pharmaceutically acceptable salt thereof.

The present disclosure further provides a method of inhibiting the interaction between menin and MLL comprising contacting the menin and MLL with a compound of Formula I′, I, I-a, I-b, or I-c, or a stereoisomer, or a pharmaceutically acceptable salt thereof.

The present disclosure further provides a method of inhibiting the interaction between menin and MLL comprising contacting the menin and MLL with a pharmaceutical composition comprising a compound of Formula I′, I, I-a, I-b, or I-c, or a pharmaceutically acceptable salt thereof, and at least one pharmaceutically acceptable carrier.

The present disclosure further provides a method of treating cancer in a patient comprising administering to the patient a compound of Formula I′, I, I-a, I-b, or I-c, a stereoisomer, or a pharmaceutically acceptable salt thereof.

The present disclosure further provides a method of treating cancer in a patient comprising administering to the patient a pharmaceutical composition comprising a compound of Formula I′, I, I-a, I-b, or I-c, a stereoisomer, or a pharmaceutically acceptable salt thereof, and at least one pharmaceutically acceptable carrier.

The present disclosure further provides a pharmaceutical composition comprising a compound of Formula I′, I, I-a, I-b, or I-c, a stereoisomer, or a pharmaceutically acceptable salt thereof, and at least one pharmaceutically acceptable carrier.

The present disclosure further provides a pharmaceutical composition comprising a salt or crystalline form of a compound of Formula I′, I, I-a, I-b, or I-c, and at least one pharmaceutically acceptable carrier.

The present disclosure further provides a compound of Formula I′, I, I-a, I-b, or I-c, wherein the compound is useful for the treatment of cancer and wherein the compound minimizes hERG binding.

In some embodiments, present disclosure further provides a method of preparing a compound herein according to a scheme of the present disclosure or synthetic description in the Examples.

In some embodiments, present disclosure further provides an intermediate useful in the preparation of any one of the compounds herein.

The details of the disclosure are set forth in the accompanying description below. Although methods and materials similar or equivalent to those described herein can be used in the practice or testing of the present application, illustrative methods and materials are now described. In the case of conflict, the present specification, including definitions, will control. In addition, the materials, methods, and examples are illustrative only and are not intended to be limiting. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### DETAILED DESCRIPTION

In some embodiments, the present disclosure is directed to a compound of Formula I′,

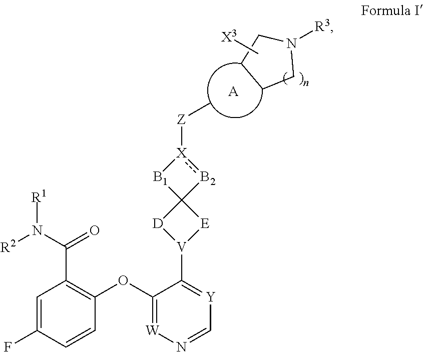

a stereoisomer, or a pharmaceutically acceptable salt thereof, wherein:
[symbol] denotes a single bond or a double bond, as valency permits;
B₁, B₂, D, and E are each independently selected from —C(R^{A1})(R^{A2})—, —C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)— wherein no more than one of B₁, B₂, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂)—;
V is N or CR^{W}, wherein R^{W} is H, halo, CN, OH, C₁-C₄ alkyl, C₁-C₄ alkoxy, amino, C₁-C₄ alkyl amino, or C₂-C₈ dialkylamino;
X is N or CR^{X}, wherein R^{X} is absent, H, halo, CN, OH, C₁-C₄ alkyl, C₁-C₄ alkoxy, amino, C₁-C₄ alkyl amino, or C₂-C₈ dialkylamino;
each R^{A1} is independently selected from absent, H, halo, C₁-C₄ alkyl, C₁-C₄ alkoxy, C₁-C₄ haloalkyl, C₁-C₄ haloalkoxy, amino, C₁-C₄ alkylamino, C₂-C₈ dialkylamino, CN, NO₂, and OH;
each R^{A2} is independently selected from H, halo, C₁-C₄ alkyl, C₁-C₄ alkoxy, C₁-C₄ haloalkyl, C₁-C₄ haloalkoxy, amino, C₁-C₄ alkylamino, C₂-C₈ dialkylamino, CN, NO₂, and OH;
each R^{A3} is independently selected from H, C₁-C₄ alkyl, C₁-C₄ alkoxy, C₁-C₄ haloalkyl, C(O)R^{z}, and C(O)OR^{z}, wherein said C₁-C₄ alkyl is optionally substituted by phenyl, C₁-C₄ alkoxy, C₁-C₄ haloalkoxy, CN, NO₂, or OH;
R^{z} is H, C₁-C₄ alkyl, or phenyl;
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R′)(R′);
Z is O, CH₂, or NH,
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4b′})C(O)(R^{4b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.

In some embodiments, when X is N in Formular I′, the atom of Z that is directly bonded with X is other than N.

In some embodiments, when X is N in Formular I′, the atom of Z that is directly bonded with X is other than O.

In some embodiments, when X is N in Formular I′, the atom of Z that is directly bonded with X is other than C.

In some embodiments, when [symbol] is a double bond, X is C.

In some embodiments, the spiro moiety represented by the below formula:

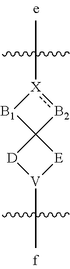

wherein e and f indicate points of attachment to the remainder of the molecule, is selected from:

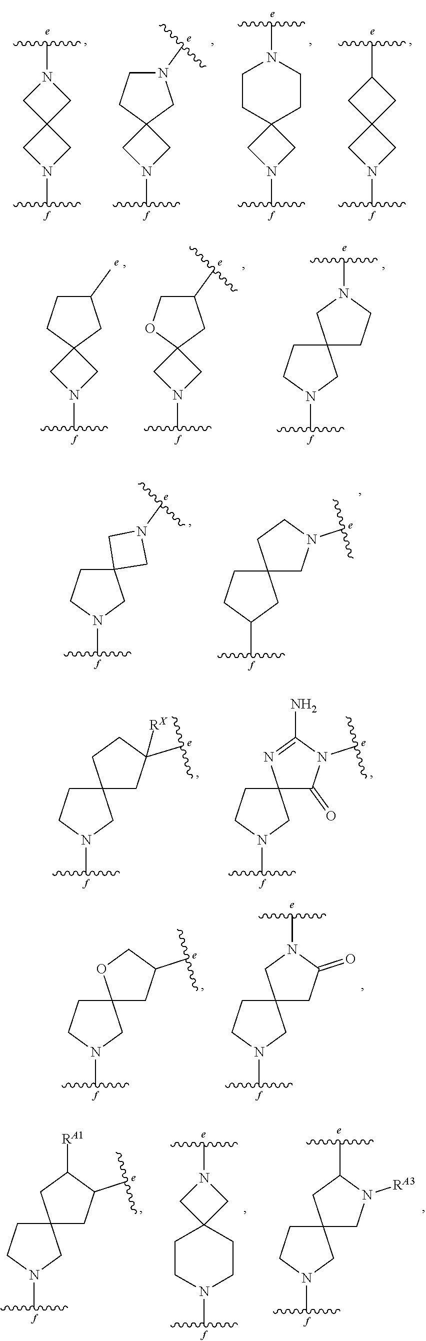

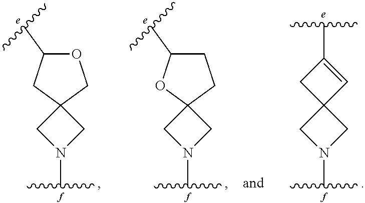 [SMILES: *C1CC2(CO1)CN(*)C2 ; *C1CCC2(CN(*)C2)O1 ; *C1=CC2(C1)CN(*)C2]

In some embodiments, the present disclosure is directed to a compound of Formula I,

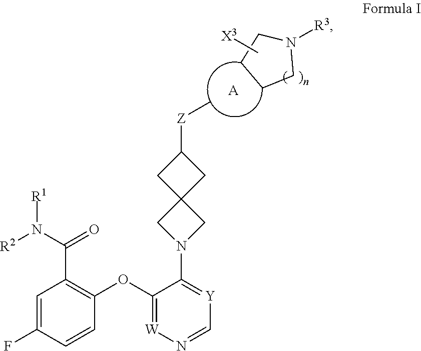

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Z is O, CH₂, or NH,
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a})(R^{4b}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{4a′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.

In some embodiments, the present disclosure is directed to a compound of Formula I-a,

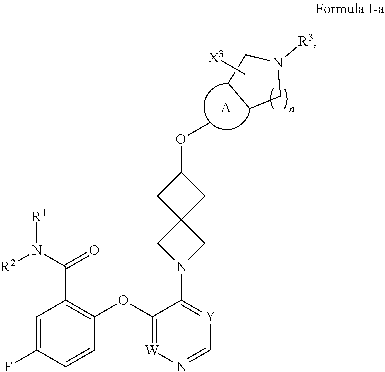

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R*), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})C(O)(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})C(O)(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.

In some embodiments, the present disclosure is directed to a compound of Formula I-a,

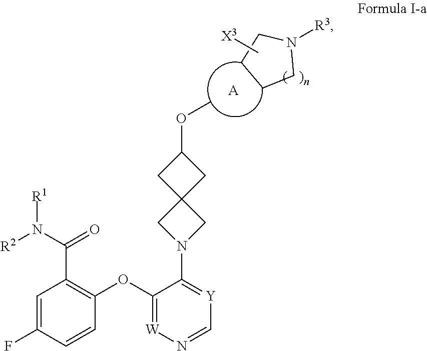

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} independently C₁-C₆ alkyl;
   (b) 3- to 12-membered heterocyclyl which has at least one nitrogen and optionally one or more other heteroatom ring members, wherein the 3- to 12-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, C₁-C₆ alkyl, or OR^{3a′}, wherein the alkyl is optionally substituted by one or more C₁-C₆ alkoxy;
R² is C₁-C₆ alkyl;
Ring A is 6- to 10-membered aryl or 6- to 10-membered heteroaryl;
X³ is H or C₁-C₆ alkyl;
R³ is H, C₁-C₆ alkyl, C(O)(R^{4a}), or C(O)O—C(R^{4a})₂—OC(O)(R^{4a}), wherein the C₁-C₆ alkyl is optionally substituted with one or more R^{3a}; or R³ forms a 3- to 12-membered heterocyclyl with the carbon atom next to the nitrogen atom to which it is connected;
each R^{3a} is independently C₁-C₆ alkoxy, C₆-C₁₀ aryl, or 3- to 12-membered heterocyclyl;
each R^{4a} is independently H or C₁-C₆ alkyl optionally substituted with C₆-C₁₀ aryl or N(R^{4a′})(R^{4b′});
each R^{3a′} is independently H, C₁-C₆ alkyl, C₁-C₆ haloalkyl, C(O)R^{3c′}, or 3- to 12-membered heterocyclyl, wherein the alkyl is optionally substituted with C₁-C₆ alkoxy;
each R^{3b′} is independently H or C₁-C₆ alkyl;
each R^{3c′} is independently C₁-C₆ alkyl;
each R^{4a′} and R^{4b′} is independently H; and
n is 1 or 2.

In some embodiments, the present disclosure is directed to a compound of Formula I-b,

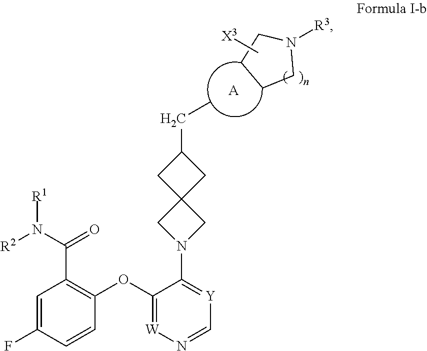

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a′})(R^{4b′});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.

In some embodiments, the present disclosure is directed to a compound of Formula I-b,

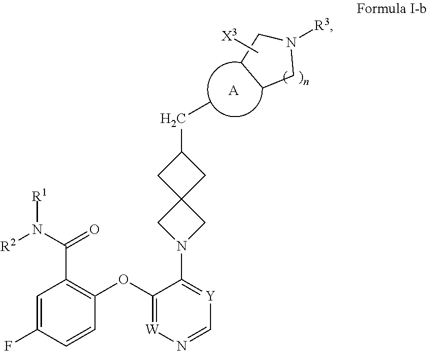

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently C₁-C₆ alkyl;
   (b) 3- to 12-membered heterocyclyl which has at least one nitrogen and optionally one or more other heteroatom ring members, wherein the 3- to 12-membered heterocyclyl is optionally substituted with one or more R^{Me}s;
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, C₁-C₆ alkyl, or OR^{3a′}, wherein the alkyl is optionally substituted by one or more C₁-C₆ alkoxy;
R² is C₁-C₆ alkyl;
Ring A is 6- to 10-membered aryl or 6- to 10-membered heteroaryl;
X³ is H or C₁-C₆ alkyl;
R³ is H, C₁-C₆ alkyl, C(O)(R^{4a}), or C(O)O—C(R^{4a})₂—OC(O)(R^{4a}), wherein the C₁-C₆ alkyl is optionally substituted with one or more R^{3a}; or R³ forms a 3- to 12-membered heterocyclyl with the carbon atom next to the nitrogen atom to which it is connected;
each R^{3a} is independently C₁-C₆ alkoxy, C₆-C₁₀ aryl, or 3- to 12-membered heterocyclyl;
each R^{4a} is independently H or C₁-C₆ alkyl optionally substituted with C₆-C₁₀ aryl or N(R^{4a′})(R^{4b′});
each R^{3a′} is independently H, C₁-C₆ alkyl, C₁-C₆ haloalkyl, C(O)R^{3c′}, or 3- to 12-membered heterocyclyl, wherein the alkyl is optionally substituted with C₁-C₆ alkoxy;
each R^{3b′} is independently H or C₁-C₆ alkyl;
each R^{3c′} is independently C₁-C₆ alkyl;
each R^{4a′} and R^{4b′} is independently H; and
n is 1 or 2.

In some embodiments, the present disclosure is directed to a compound of Formula I-c,

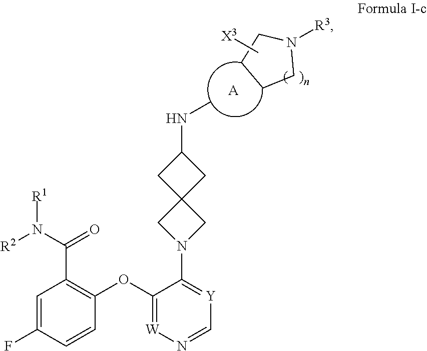

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(R^{3a′})(R^{3b′}), N(R^{3a′})(R^{3b′}), OR^{3′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a′})(R^{4b′});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4b′})C(O)(R^{4b′}), N(R^{4a′})C(O)O(R^{4b′}), C₁-C₆ alkyl that is optionally substituted with O(R^{4a′}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.

In some embodiments, the present disclosure is directed to a compound of Formula I-c,

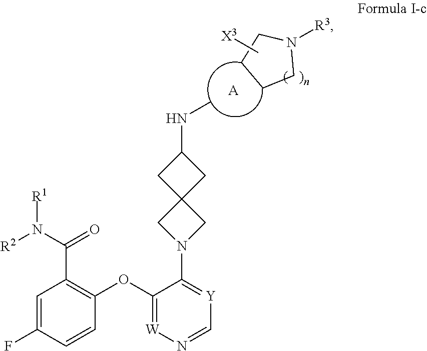

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} independently C₁-C₆ alkyl;
   (b) 3- to 12-membered heterocyclyl which has at least one nitrogen and optionally one or more other heteroatom ring members, wherein the 3- to 12-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1bs} is independently halo, C₁-C₆ alkyl, or OR^{3a′}, wherein the alkyl is optionally substituted by one or more C₁-C₆ alkoxy;
R² is C₁-C₆ alkyl;
Ring A is 6- to 10-membered aryl or 6- to 10-membered heteroaryl;
X³ is H or C₁-C₆ alkyl;
R³ is H, C₁-C₆ alkyl, C(O)(R^{4a}), or C(O)O—C(R^{4a})₂—OC(O)(R^{4a}), wherein the C₁-C₆ alkyl is optionally substituted with one or more R^{3a}; or R³ forms a 3- to 12-membered heterocyclyl with the carbon atom next to the nitrogen atom to which it is connected;
each R^{3a} is independently C₁-C₆ alkoxy, C₆-C₁₀ aryl, or 3- to 12-membered heterocyclyl;
each R^{4a} is independently H or C₁-C₆ alkyl optionally substituted with C₆-C₁₀ aryl or N(R^{4a′})(R^{4b′});
each R^{3a′} is independently H, C₁-C₆ alkyl, C₁-C₆ haloalkyl, C(O)R^{3c′}, or 3- to 12-membered heterocyclyl, wherein the alkyl is optionally substituted with C₁-C₆ alkoxy;
each R^{3b′} is independently H or C₁-C₆ alkyl;
each R^{3c′} is independently C₁-C₆ alkyl;
each R^{4a′} and R^{4b′} is independently H; and
n is 1 or 2.

### Embodiments

For any of Formulae I′, I, I-a, I-b, and I-c where applicable, the following embodiments are considered both alone and in conjunction with another where a stable compound is formed.
In some embodiments, W is N or CH.
In some embodiments, W is N.
In some embodiments, W is CH.
In some embodiments, Y is N or CH.
In some embodiments, Y is N.
In some embodiments, Y is CH.
In some embodiments, R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(O)₂R^{3a′},
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₁-C₆ alkylene-N(R^{3a′})(R^{3b′}), wherein the C₁-C₆ alkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₁-C₈ alkylene-N(R^{3a′})(R^{3b′}), wherein the C₁-C₈ alkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)R^{3a′},
each R^{3a} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₁-C₄ alkylene-N(R^{3a′})(R^{3b′}), wherein the C₁-C₄ alkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₁-C₃ alkylene-N(R^{3a′})(R^{3b′}), wherein the C₁-C₃ alkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′},
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₁-C₂ alkylene-N(R^{3a′})(R^{3b′}), wherein the C₁-C₂ alkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —CH₂—N(R^{3a′})(R^{3b′}), wherein the —CH₂— is optionally substituted with one or more R^{1as};
each R^{1as} is independently halo, oxo, C₁-C₆ alkyl, C₁-C₆ haloalkyl, C(═O)(R³), N(R^{3a′})(R^{3b′}), or S(O)₂R^{3a′},
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —(CH₂)₂—N(R^{3a′})(R^{3b′}), wherein the —(CH₂)₂— is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —(CH₂)₃—N(R^{3a′})(R^{3b′}), wherein the —(CH₂)₃— is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(O)R^{3a′},
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —(CH₂)₄—N(R^{3a′})(R^{3b′}), wherein the —(CH₂)₄— is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —(CH₂)₅—N(R^{3a′})(R^{3b′}), wherein the —(CH₂)₅— is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —(CH₂)₆—N(R^{3a′})(R^{3b′}), wherein the —(CH₂)₆— is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₃-C₁₂ cycloalkylene-N(R^{3a′})(R^{3b′}), wherein the C₃-C₁₂ cycloalkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)R^{3a′},
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₃-C₁₁ cycloalkylene-N(R^{3a′})(R^{3b′}), wherein the C₃-C₁₁ cycloalkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₃-C₁₀ cycloalkylene-N(R^{3a′})(R^{3b′}), wherein the C₃-C₁₀ cycloalkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′},
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₃-C₉ cycloalkylene-N(R^{3a′})(R^{3b′}), wherein the C₃-C₉ cycloalkylene is optionally substituted with one or more R^{1as};
each R^{1as} is independently halo, oxo, C₁-C₆ alkyl, C₁-C₆ haloalkyl, C(═O)(R^{3a′}), N(R^{3a′})R^{3b′}), or S(═O)₂R^{1as};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₃-C₈ cycloalkylene-N(R^{3a′})(R^{3b′}), wherein the C₃-C₈ cycloalkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)R^{3a′},
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₃-C₇ cycloalkylene-N(R^{3a′})(R^{3b′}), wherein the C₃-C₇ cycloalkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₃-C₆ cycloalkylene-N(R^{3a′})(R^{3b′}), wherein the C₃-C₆ cycloalkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′},
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₃-C₅ cycloalkylene-N(R^{3a′})(R^{3b′}), wherein the C₃-C₈ cycloalkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₃-C₄ cycloalkylene-N(R^{3a′})(R^{3b′}), wherein the C₃-C₄ cycloalkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(O)R^{3a′},
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₃ cycloalkylene-N(R^{3a′})(R^{3b′}), wherein the —C₃ cycloalkylene- is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{1as};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₄ cycloalkylene-N(R^{3a′})(R^{3b′}), wherein the —C₄ cycloalkylene- is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(O)R^{3a′},
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₅ cycloalkylene-N(R^{3a′})(R^{3b′}), wherein the —C₅ cycloalkylene- is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₆ cycloalkylene-N(R^{3a′})(R^{3b′}), wherein the —C₆ cycloalkylene- is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₇ cycloalkylene-N(R^{3a′})(R^{3b′}), wherein the —C₇ cycloalkylene- is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R¹ is independently H, C₁-C₆ alkyl, C₁-C₆ haloalkyl, C₃-C₁₂ cycloalkyl, or 3- to 12-membered heterocyclyl.

In some embodiments, R¹ is —C₈ cycloalkylene-N(R^{3a′})(R^{3b′}), wherein the —C₈ cycloalkylene- is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)R^{3a′},
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₉ cycloalkylene-N(R^{3a′})(R^{3b′}), wherein the —C₉ cycloalkylene- is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₁₀ cycloalkylene-N(R^{3a′})(R^{3b′}), wherein the —C₁₀ cycloalkylene- is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′},
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R¹ is independently H, C₁-C₆ alkyl, C₁-C₆ haloalkyl, C₃-C₁₂ cycloalkyl, or 3- to 12-membered heterocyclyl.

In some embodiments, R¹ is —C₁₁ cycloalkylene-N(R^{3a′})(R^{3b′}), wherein the —C₁₁ cycloalkylene- is optionally substituted with one or more R^{1as};
each R^{1as} is independently halo, oxo, C₁-C₆ alkyl, C₁-C₆ haloalkyl, C(═O)(R^{3a′}), N(R^{3a′})R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is —C₁₂ cycloalkylene-N(R^{3a′})(R^{3b′}), wherein the —C₁₂ cycloalkylene- is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is 3- to 12-membered heterocyclyl which has at least one nitrogen and optionally one or more other heteroatom ring members, wherein the 3- to 12-membered heterocyclyl is optionally substituted with one or more R^{1bs},
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′},
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is 3- to 11-membered heterocyclyl which has at least one nitrogen and optionally one or more other heteroatom ring members, wherein the 3- to 11-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is 3- to 10-membered heterocyclyl which has at least one nitrogen and optionally one or more other heteroatom ring members, wherein the 3- to 10-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{1bs} is independently H, C₁-C₆ alkyl, C₁-C₆ haloalkyl, C₃-C₁₂ cycloalkyl, or 3- to 12-membered heterocyclyl.

In some embodiments, R¹ is 3- to 9-membered heterocyclyl which has at least one nitrogen and optionally one or more other heteroatom ring members, wherein the 3- to 9-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R¹ is independently H, C₁-C₆ alkyl, C₁-C₆ haloalkyl, C₃-C₁₂ cycloalkyl, or 3- to 12-membered heterocyclyl.

In some embodiments, R¹ is 3- to 8-membered heterocyclyl which has at least one nitrogen and optionally one or more other heteroatom ring members, wherein the 3- to 8-membered heterocyclyl is optionally substituted with one or more R^{1bs};
each R^{1bs} is independently halo, oxo, OR^{3′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′},
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is 3- to 7-membered heterocyclyl which has at least one nitrogen and optionally one or more other heteroatom ring members, wherein the 3- to 7-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′},)(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is 3- to 6-membered heterocyclyl which has at least one nitrogen and optionally one or more other heteroatom ring members, wherein the 3- to 6-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is 3- to 5-membered heterocyclyl which has at least one nitrogen and optionally one or more other heteroatom ring members, wherein the 3- to 5-membered heterocyclyl is optionally substituted with one or more R^{1bs};
each R^{1bs} is independently halo, oxo, OR³, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is 3- to 4-membered heterocyclyl which has at least one nitrogen and optionally one or more other heteroatom ring members, wherein the 3- to 4-membered heterocyclyl is optionally substituted with one or more R^{1bs};
each R^{1bs} is independently halo, oxo, OR^{3′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′},
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is 12-membered heterocyclyl which has at least one nitrogen and optionally one or more other heteroatom ring members, wherein the 12-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is 11-membered heterocyclyl which has at least one nitrogen and optionally one or more other heteroatom ring members, wherein the 11-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is 10-membered heterocyclyl which has at least one nitrogen and optionally one or more other heteroatom ring members, wherein the 10-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is 9-membered heterocyclyl which has at least one nitrogen and optionally one or more other heteroatom ring members, wherein the 9-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′},
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is 8-membered heterocyclyl which has at least one nitrogen and optionally one or more other heteroatom ring members, wherein the 8-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)R^{3a′};
each R^{3a} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is 7-membered heterocyclyl which has at least one nitrogen and optionally one or more other heteroatom ring members, wherein the 7-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is 6-membered heterocyclyl which has at least one nitrogen and optionally one or more other heteroatom ring members, wherein the 6-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is 5-membered heterocyclyl which has at least one nitrogen and optionally one or more other heteroatom ring members, wherein the 5-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is 4-membered heterocyclyl which has at least one nitrogen and optionally one or more other heteroatom ring members, wherein the 4-membered heterocyclyl is optionally substituted with one or more R^{1bs};
each R^{1bs} is independently halo, oxo, OR^{3′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′},
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is 3-membered heterocyclyl which has at least one nitrogen and optionally one or more other heteroatom ring members, wherein the 3-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)R^{3a′};
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is C₃-C₁₁ cycloalkyl substituted with one or more 3- to 12-membered heterocyclyl which has at least one nitrogen and optionally one or more other heteroatom ring members; and wherein each of the C₃-C₁₁ cycloalkyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is C₃-C₁₀ cycloalkyl substituted with one or more 3- to 12-membered heterocyclyl which has at least one nitrogen and optionally one or more other heteroatom ring members; and wherein each of the C₃-C₁₀ cycloalkyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is C₃-C₉ cycloalkyl substituted with one or more 3- to 12-membered heterocyclyl which has at least one nitrogen and optionally one or more other heteroatom ring members; and wherein each of the C₃-C₉ cycloalkyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′} or S(═O)₂R^{3a′}, wherein the alkyl or haloalkyl is optionally substituted by one or more C₁-C₆ alkoxy;
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R¹ is independently H, C₁-C₆ alkyl, C₁-C₆ haloalkyl, C₃-C₁₂ cycloalkyl, or 3- to 12-membered heterocyclyl.

In some embodiments, R¹ is C₃-C₈ cycloalkyl substituted with one or more 3- to 12-membered heterocyclyl which has at least one nitrogen and optionally one or more other heteroatom ring members; and wherein each of the C₃-C₈ cycloalkyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is C₃-C₇ cycloalkyl substituted with one or more 3- to 12-membered heterocyclyl which has at least one nitrogen and optionally one or more other heteroatom ring members; and wherein each of the C₃-C₇ cycloalkyl and the 3- to 12-membered heterocyclyl is optionally substituted with one or more R^{1cs};
each R^{1cs} is independently halo, oxo, C₁-C₆ alkyl, C₁-C₆ haloalkyl, C(═OX(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is C₃-C₆ cycloalkyl substituted with one or more 3- to 12-membered heterocyclyl which has at least one nitrogen and optionally one or more other heteroatom ring members; and wherein each of the C₃-C₆ cycloalkyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is C₃-C₈ cycloalkyl substituted with one or more 3- to 12-membered heterocyclyl which has at least one nitrogen and optionally one or more other heteroatom ring members; and wherein each of the C₃-C₅ cycloalkyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3′}, or S(═O)₂R^{3a′}, wherein the alkyl or haloalkyl is optionally substituted by one or more C₁-C₆ alkoxy;
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is C₃-C₄ cycloalkyl substituted with one or more 3- to 12-membered heterocyclyl which has at least one nitrogen and optionally one or more other heteroatom ring members; and wherein each of the C₃-C₄ cycloalkyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R¹ is independently H, C₁-C₆ alkyl, C₁-C₆ haloalkyl, C₃-C₁₂ cycloalkyl, or 3- to 12-membered heterocyclyl.

In some embodiments, R¹ is cyclopropyl substituted with one or more 3- to 12-membered heterocyclyl which has at least one nitrogen and optionally one or more other heteroatom ring members; and wherein each of the cyclopropyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is cyclobutyl substituted with one or more 3- to 12-membered heterocyclyl which has at least one nitrogen and optionally one or more other heteroatom ring members; and wherein each of the cyclobutyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is cyclopentyl substituted with one or more 3- to 12-membered heterocyclyl which has at least one nitrogen and optionally one or more other heteroatom ring members; and wherein each of the cyclopentyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
each R^{3a′} is independently H, C₁-C₆ alkyl, C₁-C₆ haloalkyl, C(O)R^{3a′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is cyclohexyl substituted with one or more 3- to 12-membered heterocyclyl which has at least one nitrogen and optionally one or more other heteroatom ring members; and wherein each of the cyclohexyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R¹ is independently H, C₁-C₆ alkyl, C₁-C₆ haloalkyl, C₃-C₁₂ cycloalkyl, or 3- to 12-membered heterocyclyl.

In some embodiments, R¹ is cycloheptyl substituted with one or more 3- to 12-membered heterocyclyl which has at least one nitrogen and optionally one or more other heteroatom ring members; and wherein each of the cycloheptyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is cyclooctyl substituted with one or more 3- to 12-membered heterocyclyl which has at least one nitrogen and optionally one or more other heteroatom ring members; and wherein each of the cyclooctyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is cyclononyl substituted with one or more 3- to 12-membered heterocyclyl which has at least one nitrogen and optionally one or more other heteroatom ring members; and wherein each of the cyclononyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

In some embodiments, R¹ is cyclodecyl substituted with one or more 3- to 12-membered heterocyclyl which has at least one nitrogen and optionally one or more other heteroatom ring members; and wherein each of the cyclodecyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3′}, or S(═O)₂R^{3a′}, wherein the alkyl or haloalkyl is optionally substituted by one or more C₁-C₆ alkoxy;
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl; and
each R^{3c′} is independently H, C₁-C₆ alkyl, C₁-C₆ haloalkyl, C₃-C₁₂ cycloalkyl, or 3- to 12-membered heterocyclyl.

### In Some Embodiments, R¹ is:

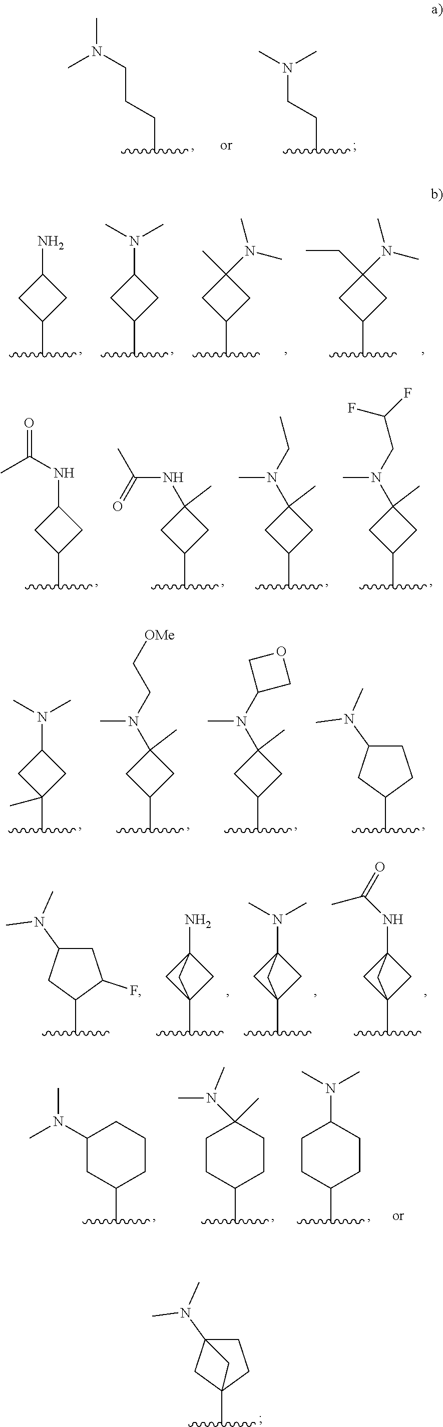

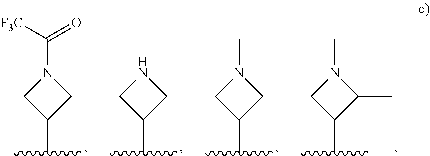

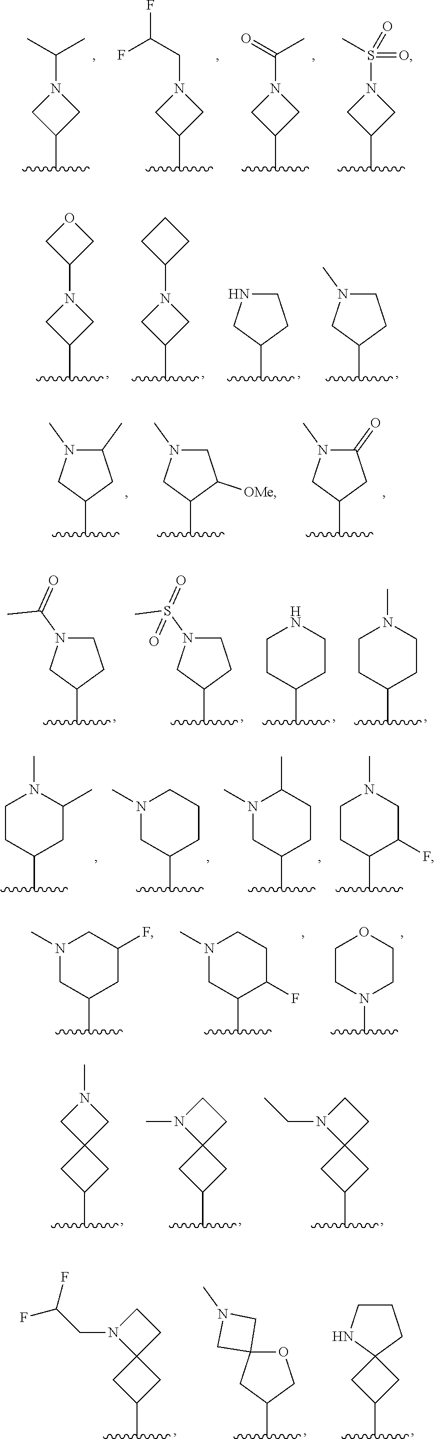

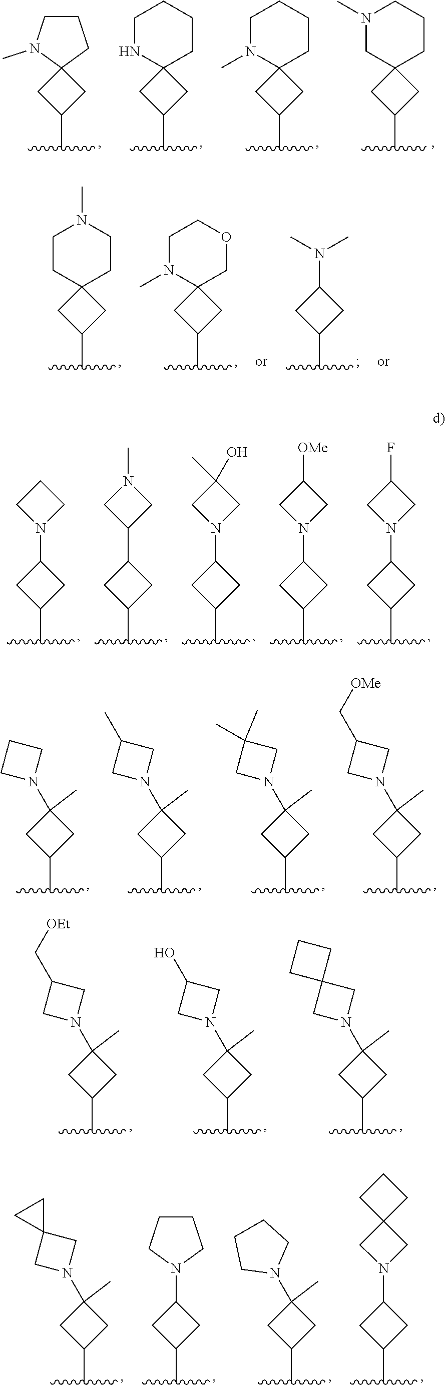

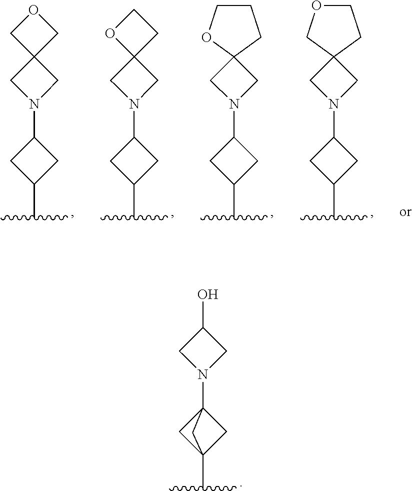

In some embodiments, R¹ is:

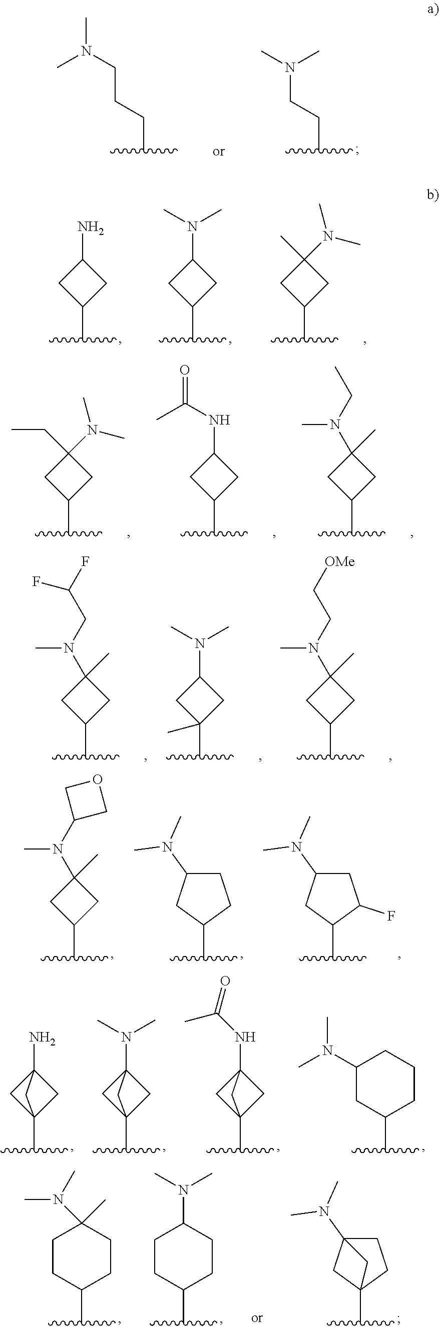

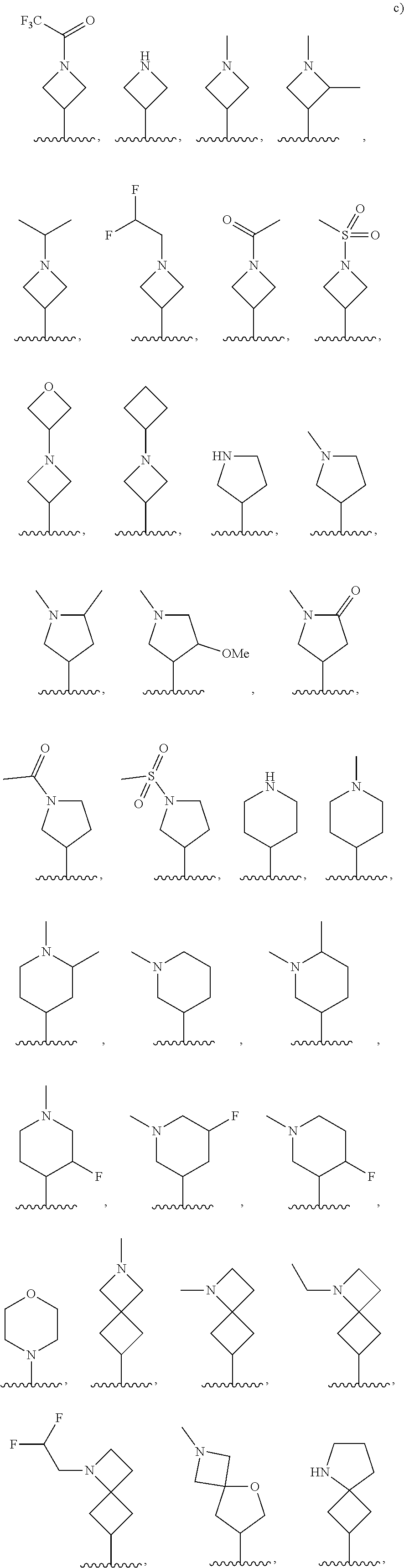

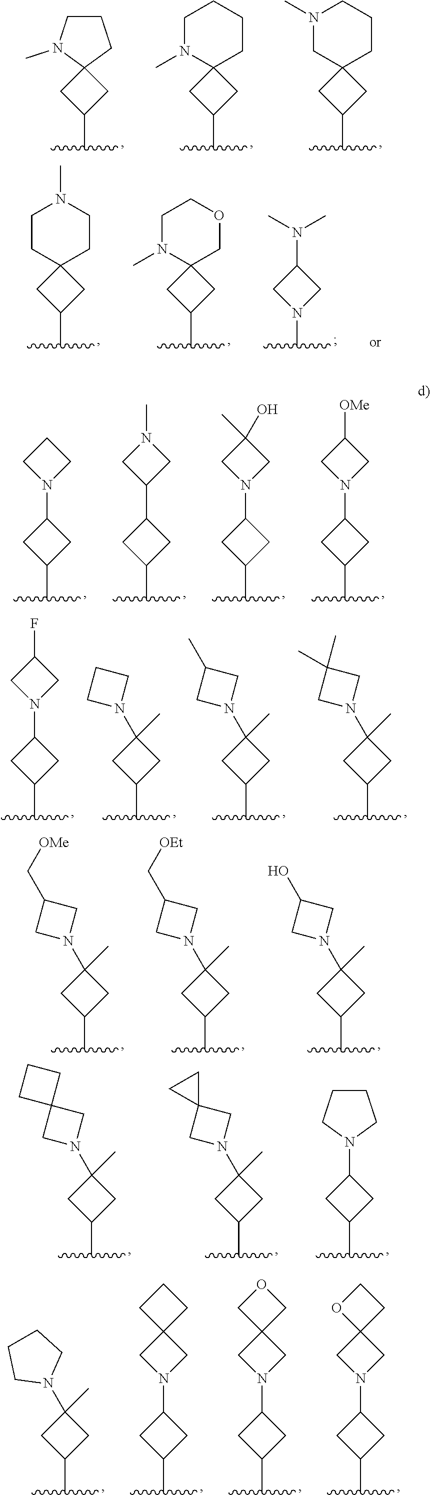

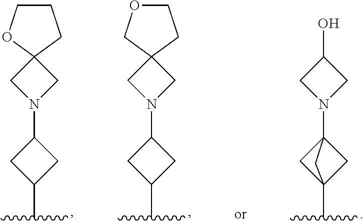 [SMILES: *C1CC(N2CC3(CCCO3)C2)C1 ; *C1CC(N2CC3(CCOC3)C2)C1 ; *C12CC(N3CC(O)C3)(C1)C2]

In some embodiments, R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′}), wherein each R^{4a′} and R^{4b′} is independently H, C₁-C₆ alkyl, C₃-C₁₂ cycloalkyl, or 3- to 12-membered heterocyclyl.

In some embodiments, R² is H.

In some embodiments, R² is C₁-C₆ alkyl.

In some embodiments, R² is C₁-C₈ alkyl.

In some embodiments, R² is C₁-C₄ alkyl.

In some embodiments, R² is C₁-C₃ alkyl.

In some embodiments, R² is C₁-C₂ alkyl.

In some embodiments, R² is methyl.

In some embodiments, R² is ethyl.

In some embodiments, R² is propyl.

In some embodiments, R² is isopropyl.

In some embodiments, R² is butyl.

In some embodiments, R² is tert-butyl.

In some embodiments, R² is pentyl.

In some embodiments, R² is hexyl.

In some embodiments, Z is O, CH₂, or NH.

In some embodiments, Z is O.

In some embodiments, Z is CH₂.

In some embodiments, Z is NH.

In some embodiments, Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy.

In some embodiments, Ring A is 6- to 10-membered aryl optionally substituted with one or more C₁-C₆ alkyl, halo, OH, CN, or C₁-C₆ alkoxy.

In some embodiments, Ring A is 6- to 9-membered aryl optionally substituted with one or more C₁-C₆ alkyl, halo, OH, CN, or C₁-C₆ alkoxy.

In some embodiments, Ring A is 6- to 8-membered aryl optionally substituted with one or more C₁-C₆ alkyl, halo, OH, CN, or C₁-C₆ alkoxy.

In some embodiments, Ring A is 6- to 7-membered aryl optionally substituted with one or more C₁-C₆ alkyl, halo, OH, CN, or C₁-C₆ alkoxy.

In some embodiments, Ring A is a phenyl ring.

In some embodiments, Ring A is a phenyl ring optionally substituted with one or more C₁-C₆ alkyl, halo, OH, CN, or C₁-C₆ alkoxy.

In some embodiments, Ring A is 6- to 10-membered heteroaryl optionally substituted with one or more C₁-C₆ alkyl, halo, OH, CN, or C₁-C₆ alkoxy.

In some embodiments, Ring A is 6- to 9-membered heteroaryl optionally substituted with one or more C₁-C₆ alkyl, halo, OH, CN, or C₁-C₆ alkoxy.

In some embodiments, Ring A is 6- to 8-membered heteroaryl optionally substituted with one or more C₁-C₆ alkyl, halo, OH, CN, or C₁-C₆ alkoxy.

In some embodiments, Ring A is 6- to 7-membered heteroaryl optionally substituted with one or more C₁-C₆ alkyl, halo, OH, CN, or C₁-C₆ alkoxy.

In some embodiments, Ring A is a pyridine ring optionally substituted with one or more C₁-C₆ alkyl, halo, OH, CN, or C₁-C₆ alkoxy.

In some embodiments, Ring A is a pyridine ring.

In some embodiments, Ring A is

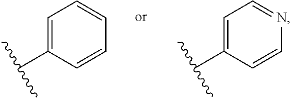 [SMILES: *c1ccccc1 ; "or" ; *c1ccncc1]

wherein

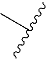

represents a bond to Z.

In some embodiments,

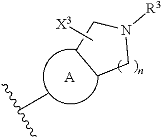

is

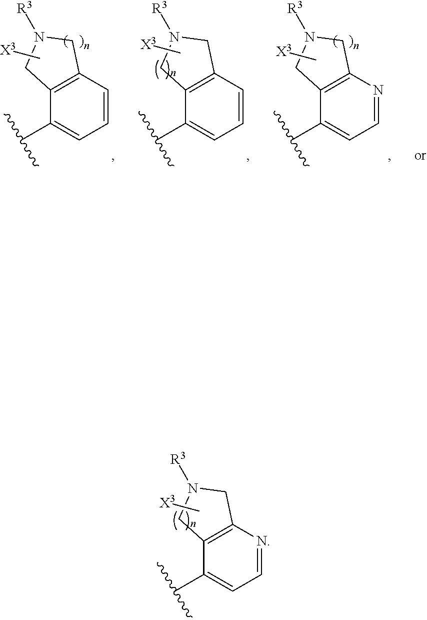

In some embodiments,

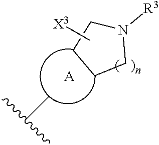

is

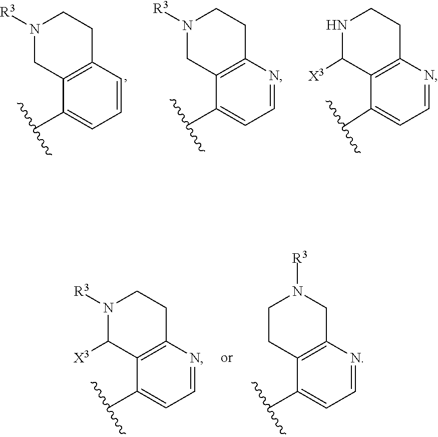

In some embodiments,

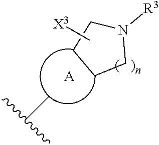

is

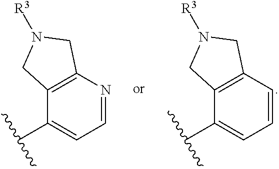

In some embodiments,

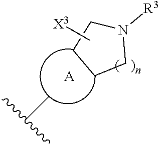

is

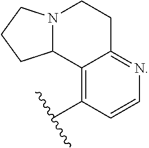 [SMILES: *c1ccnc2c1C1CCCN1CC2]

In some embodiments, X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b}); wherein:
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}, R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′}); and
each R^{4a′} and R^{4b′} is independently H, C₁-C₆ alkyl, C₃-C₁₂ cycloalkyl, or 3- to 12-membered heterocyclyl.

In some embodiments, X³ is H.

In some embodiments, X³ is C₁-C₆ alkyl.

In some embodiments, X³ is C₁-C₅ alkyl.

In some embodiments, X³ is C₁-C₄ alkyl.

In some embodiments, X³ is C₁-C₃ alkyl.

In some embodiments, X³ is C₁-C₂ alkyl.

In some embodiments, X³ is methyl.

In some embodiments, X³ is ethyl.

In some embodiments, X³ is propyl.

In some embodiments, X³ is isopropyl.

In some embodiments, X³ is butyl.

In some embodiments, X³ is tert-butyl.

In some embodiments, X³ is C₁-C₆ alkyl substituted with one or more —OH.

In some embodiments, X³ is C₁-C₅ alkyl substituted with one or more —OH.

In some embodiments, X³ is C₁-C₄ alkyl substituted with one or more —OH.

In some embodiments, X³ is C₁-C₃ alkyl substituted with one or more —OH.

In some embodiments, X³ is C₁-C₂ alkyl substituted with one or more —OH.

In some embodiments, X³ is C₁-C₆ alkyl substituted with one or more —OH.

In some embodiments, X³ is C₁-C₅ alkyl substituted with one or more —OH.

In some embodiments, X³ is C₁-C₄ alkyl substituted with one or more —OH.

In some embodiments, X³ is C₁-C₃ alkyl substituted with one or more —OH.

In some embodiments, X³ is C₁-C₂ alkyl substituted with one or more —OH.

In some embodiments, X³ is —CH₂(OH), —CH₂—CH₂(OH), or —CH₂—CH₂—CH₂(OH).

In some embodiments, X³ is C₁-C₆ alkyl substituted with one or more OR^{4a}; wherein each R^{4a} is independently H, CN, or C₁-C₆ alkyl optionally substituted with C₆-C₁₀ aryl or N(R^{4a′})(R^{4b′}), wherein each R^{4a′} and R^{4b′} is independently H, C₁-C₆ alkyl, C₃-C₁₂ cycloalkyl, or 3- to 12-membered heterocyclyl.

In some embodiments, X³ is C₁-C₅ alkyl substituted with one or more OR^{4a}; wherein each R^{4a} is independently H, CN, or C₁-C₆ alkyl optionally substituted with C₆-C₁₀ aryl or N(R^{4a′})(R^{4b′}), wherein each R^{4a′} and R^{4b′} is independently H, C₁-C₆ alkyl, C₃-C₁₂ cycloalkyl, or 3- to 12-membered heterocyclyl.

In some embodiments, X³ is C₁-C₄ alkyl substituted with one or more OR^{4a}; wherein each R¹ is independently H, CN, or C₁-C₆ alkyl optionally substituted with C₆-C₁₀ aryl or N(R^{4a′})(R^{4b′}), wherein each R^{4a′} and R^{4b′} is independently H, C₁-C₆ alkyl, C₃-C₁₂ cycloalkyl, or 3- to 12-membered heterocyclyl.

In some embodiments, X³ is C₁-C₃ alkyl substituted with one or more OR; wherein each R⁴ is independently H, CN, or C₁-C₆ alkyl optionally substituted with C₆-C₁₀ aryl or N(R^{4a′})(R^{4b′}), wherein each R^{4a′} and R^{4b′} is independently H, C₁-C₆ alkyl, C₃-C₁₂ cycloalkyl, or 3- to 12-membered heterocyclyl.

In some embodiments, X³ is C₁-C₂ alkyl substituted with one or more OR; wherein each R¹ is independently H, CN, or C₁-C₆ alkyl optionally substituted with C₆-C₁₀ aryl or N(R^{4a′})(R^{4b′}), wherein each R^{4a′} and R^{4b′} is independently H, C₁-C₆ alkyl, C₃-C₁₂ cycloalkyl, or 3- to 12-membered heterocyclyl.

In some embodiments, X³ is C₁-C₆ alkyl substituted with one or more oxo.

In some embodiments, X³ is C₁-C₈ alkyl substituted with one or more oxo.

In some embodiments, X³ is C₁-C₄ alkyl substituted with one or more oxo.

In some embodiments, X³ is C₁-C₃ alkyl substituted with one or more oxo.

In some embodiments, X³ is C₁-C₂ alkyl substituted with one or more oxo.

In some embodiments, X³ is C₁-C₆ alkyl substituted with one or more CN.

In some embodiments, X³ is C₁-C₈ alkyl substituted with one or more CN.

In some embodiments, X³ is C₁-C₄ alkyl substituted with one or more CN.

In some embodiments, X³ is C₁-C₃ alkyl substituted with one or more CN.

In some embodiments, X³ is C₁-C₂ alkyl substituted with one or more CN.

In some embodiments, X³ is C₁-C₆ alkyl substituted with one or more C(═O)N(R^{4a})(R^{4b}); wherein:
each R^{4a} is independently H, CN, or C₁-C₆ alkyl optionally substituted with C₆-C₁₀ aryl or N(R^{4a′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′}); and
each R^{4a′} and R^{4b′} is independently H, C₁-C₆ alkyl, C₃-C₁₂ cycloalkyl, or 3- to 12-membered heterocyclyl.

In some embodiments, X³ is C₁-C₈ alkyl substituted with one or more C(═O)N(R^{4a′})(R^{4b′}); wherein:
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′}); and
each R^{4a′} and R^{4b′} is independently H, C₁-C₆ alkyl, C₃-C₁₂ cycloalkyl, or 3- to 12-membered heterocyclyl.

In some embodiments, X³ is C₁-C₄ alkyl substituted with one or more C(═O)N(R^{4a})(R^{4b}); wherein:
each R^{4a} is independently H, CN, or C₁-C₆ alkyl optionally substituted with C₆-C₁₀ aryl or N(R^{4a′})(R^{4b′});
each R^{b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′), C}₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′}); and
each R^{4a′} and R^{4b′} is independently H, C₁-C₆ alkyl, C₃-C₁₂ cycloalkyl, or 3- to 12-membered heterocyclyl.

In some embodiments, X³ is C₁-C₃ alkyl substituted with one or more C(═O)N(R^{4a})(R^{4b}); wherein:
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′}); and
each R^{4a′} and R^{4b′} is independently H, C₁-C₆ alkyl, C₃-C₁₂ cycloalkyl, or 3- to 12-membered heterocyclyl.

In some embodiments, X³ is C₁-C₂ alkyl substituted with one or more C(═O)N(R^{4a})(R^{4b}); wherein:
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′}); and
each R^{4a′} and R^{4b′} is independently H, C₁-C₆ alkyl, C₃-C₁₂ cycloalkyl, or 3- to 12-membered heterocyclyl.

In some embodiments, X³ is C₁-C₈ alkyl substituted with one or more N(R^{4a})(R^{4b}); wherein:
each R^{4a} is independently H, CN, or C₁-C₆ alkyl optionally substituted with C₆-C₁₀ aryl or N(R^{4a})(R^{4b});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a})(R^{4b}); and
each R^{4a′} and R^{4b′} is independently H, C₁-C₆ alkyl, C₃-C₁₂ cycloalkyl, or 3- to 12-membered heterocyclyl.

In some embodiments, X³ is C₁-C₅ alkyl substituted with one or more N(R^{4a})(R^{4b}); wherein:
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′}); and
each R^{4a′} and R^{4b′} is independently H, C₁-C₆ alkyl, C₃-C₁₂ cycloalkyl, or 3- to 12-membered heterocyclyl.

In some embodiments, X³ is C₁-C₄ alkyl substituted with one or more N(R^{4a})(R^{4b}); wherein:
each R^{4a} is independently H, CN, or C₁-C₆ alkyl optionally substituted with C₆-C₁₀ aryl or NR^{4a′}R^{4b′;}
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′}); and
each R^{4a′} and R^{4b′} is independently H, C₁-C₆ alkyl, C₃-C₁₂ cycloalkyl, or 3- to 12-membered heterocyclyl.

In some embodiments, X³ is C₁-C₃ alkyl substituted with one or more N(R^{4a′})(R^{4b′}); wherein:
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′}); and
each R^{4a′} and R^{4b′} is independently H, C₁-C₆ alkyl, C₃-C₁₂ cycloalkyl, or 3- to 12-membered heterocyclyl.

In some embodiments, X³ is C₁-C₂ alkyl substituted with one or more N(R^{4a′})(R^{4b′}); wherein:
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′}); and
each R^{4a′} and R^{4b′} is independently H, C₁-C₆ alkyl, C₃-C₁₂ cycloalkyl, or 3- to 12-membered heterocyclyl.

In some embodiments, R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; wherein:
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or NR^{4a′}R^{4b′;}
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′}); and
each R^{4a′} and R^{4b′} is independently H, C₁-C₆ alkyl, C₃-C₁₂ cycloalkyl, or 3- to 12-membered heterocyclyl.

In some embodiments, R³ is H.

In some embodiments, R³ is C₁-C₆ alkyl.

In some embodiments, R³ is C₁-C₅ alkyl.

In some embodiments, R³ is C₁-C₄ alkyl.

In some embodiments, R³ is C₁-C₃ alkyl.

In some embodiments, R³ is C₁-C₂ alkyl.

In some embodiments, R³ is methyl.

In some embodiments, R³ is ethyl.

In some embodiments, R³ is propyl.

In some embodiments, R³ is isopropyl.

In some embodiments, R³ is butyl.

In some embodiments, R³ is tert-butyl.

In some embodiments, R³ is C₁-C₆ alkyl substituted with one or more R^{3a}.

In some embodiments, R³ is C₁-C₅ alkyl substituted with one or more R^{3a}.

In some embodiments, R³ is C₁-C₄ alkyl substituted with one or more R³.

In some embodiments, R³ is C₁-C₃ alkyl substituted with one or more R^{3a}.

In some embodiments, R³ is C₁-C₂ alkyl substituted with one or more R^{3a}.

In some embodiments, R³ is methyl optionally substituted with one OR^{4a}. In some embodiments, R^{4a} is methyl.

In some embodiments, R³ is ethyl optionally substituted with one OR^{4a}. In some embodiments, R^{4a} is methyl.

In some embodiments, R³ is propyl optionally substituted with one OR^{4a}. In some embodiments, R^{4a} is methyl.

In some embodiments, R³ is isopropyl optionally substituted with one OR^{4a}. In some embodiments, R^{4a} is methyl.

In some embodiments, R³ is butyl optionally substituted with one OR^{4a}. In some embodiments, R^{4a} is methyl.

In some embodiments, R³ is tert-butyl optionally substituted with one OR^{4a}. In some embodiments, R^{4a} is methyl.

In some embodiments, R³ is pentyl optionally substituted with one OR^{4a}. In some embodiments, R^{4a} is methyl.

In some embodiments, R³ is hexyl optionally substituted with one OR^{4a}. In some embodiments, R^{4a} is methyl.

In some embodiments, R³ is C₂-C₆ alkenyl optionally substituted with one or more R^{3a}.

In some embodiments, R³ is C₂-C₅ alkenyl optionally substituted with one or more R^{3a}.

In some embodiments, R³ is C₂-C₄ alkenyl optionally substituted with one or more R^{3a}.

In some embodiments, R³ is C₂-C₃ alkenyl optionally substituted with one or more R^{3a}.

In some embodiments, R³ is C₂-C₆ alkynyl optionally substituted with one or more R^{3a}.

In some embodiments, R³ is C₂-C₅ alkynyl optionally substituted with one or more R^{3a}.

In some embodiments, R³ is C₂-C₄ alkynyl optionally substituted with one or more R^{3a}.

In some embodiments, R³ is C₂-C₃ alkynyl optionally substituted with one or more R^{3a}.

In some embodiments, R³ is C₁-C₆ alkoxy optionally substituted with one or more R^{3a}.

In some embodiments, R³ is C₁-C₅ alkoxy optionally substituted with one or more R^{3a}.

In some embodiments, R³ is C₁-C₄ alkoxy optionally substituted with one or more R^{3a}.

In some embodiments, R³ is C₁-C₃ alkoxy optionally substituted with one or more R^{3a}.

In some embodiments, R³ is C₁-C₂ alkoxy optionally substituted with one or more R^{3a}.

In some embodiments, R³ is a C₃-C₁₂ cycloalkyl optionally substituted with one or more R^{3a}.

In some embodiments, R³ is a C₃-C₁₁ cycloalkyl optionally substituted with one or more R^{3a}.

In some embodiments, R³ is a C₃-C₁₀ cycloalkyl optionally substituted with one or more R^{3a}.

In some embodiments, R³ is a C₃-C₉ cycloalkyl optionally substituted with one or more R^{3a}.

In some embodiments, R³ is a C₃-C₈ cycloalkyl optionally substituted with one or more R^{3a}.

In some embodiments, R³ is a C₃-C₇ cycloalkyl optionally substituted with one or more R^{3a}.

In some embodiments, R³ is a C₃-C₆ cycloalkyl optionally substituted with one or more R^{3a}.

In some embodiments, R³ is a C₃-C₅ cycloalkyl optionally substituted with one or more R^{3a}.

In some embodiments, R³ is a C₃-C₄ cycloalkyl optionally substituted with one or more R^{3a}.

In some embodiments, R³ is a C₆-C₁₀ aryl optionally substituted with one or more R^{3a}.

In some embodiments, R³ is a C₆-C₉ aryl optionally substituted with one or more R^{3a}.

In some embodiments, R³ is a C₆-C₈ aryl optionally substituted with one or more R^{3a}.

In some embodiments, R³ is a C₆-C₇ aryl optionally substituted with one or more R^{3a}.

In some embodiments, R³ is a 5- to 10-membered heteroaryl optionally substituted with one or more R^{3a}.

In some embodiments, R³ is a 5- to 9-membered heteroaryl optionally substituted with one or more R^{3a}.

In some embodiments, R³ is a 5- to 8-membered heteroaryl optionally substituted with one or more R^{3a}.

In some embodiments, R³ is a 5- to 7-membered heteroaryl optionally substituted with one or more R^{3a}.

In some embodiments, R³ is a 5- to 6-membered heteroaryl optionally substituted with one or more R^{3a}.

In some embodiments, R³ is a 3- to 12-membered heterocyclyl optionally substituted with one or more R^{3a}.

In some embodiments, R³ is a 3- to 11-membered heterocyclyl optionally substituted with one or more R^{3a}.

In some embodiments, R³ is a 3- to 10-membered heterocyclyl optionally substituted with one or more R^{3a}.

In some embodiments, R³ is a 3- to 9-membered heterocyclyl optionally substituted with one or more R^{3a}.

In some embodiments, R³ is a 3- to 8-membered heterocyclyl optionally substituted with one or more R^{3a}.

In some embodiments, R³ is a 3- to 7-membered heterocyclyl optionally substituted with one or more R^{3a}.

In some embodiments, R³ is a 3- to 6-membered heterocyclyl optionally substituted with one or more R^{3a}.

In some embodiments, R³ is a 3- to 5-membered heterocyclyl optionally substituted with one or more R^{3a}.

In some embodiments, R³ is a 3- to 4-membered heterocyclyl optionally substituted with one or more R^{3a}.

In some embodiments, R³ is C(O)(R^{4a}), wherein:
R^{4a} is H, CN, or C₁-C₆ alkyl optionally substituted with C₆-C₁₀ aryl or N(R^{4a′})(R^{4b′}); and
each (R^{4a′} and R^{4b′} is independently H, C₁-C₆ alkyl, C₃-C₁₂ cycloalkyl, or 3- to 12-membered heterocyclyl.

In some embodiments, R³ is

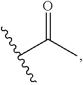 [SMILES: *C(C)=O]

In some embodiments, R³ is C(O)O—C(R^{4a})₂—OC(O)R^{4a}), wherein:
each R^{4a} is independently H, CN, or C₁-C₆ alkyl optionally substituted with C₆-C₁₀ aryl or N(R^{4a′})(R^{4b′}); and
each (R^{4a′} and R^{4b′} is independently H, C₁-C₆ alkyl, C₃-C₁₂ cycloalkyl, or 3- to 12-membered heterocyclyl.

In some embodiments, R³ is

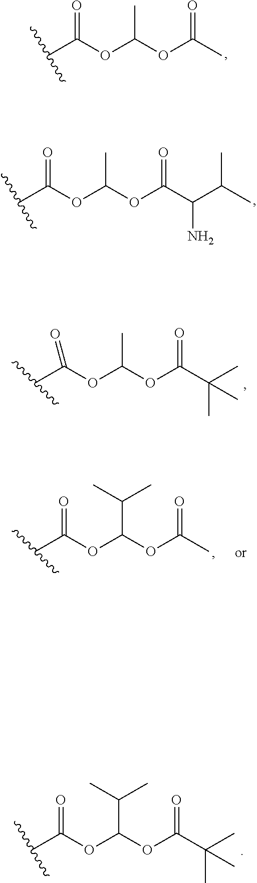

In some embodiments, R³ is C₁-C₆ alkyl substituted with one or more R^{3a}, wherein:
each R^{3a} is independently OR^{4a}, C₆-C₁₀ aryl, or 3- to 12-membered heterocyclyl; and
each R^{4a} is independently H, CN, or C₁-C₆ alkyl.

In some embodiments, R³ is C₁-C₈ alkyl substituted with one or more R^{3′}, wherein:
each R^{3a} is independently OR^{4a}, C₆-C₁₀ aryl, or 3- to 12-membered heterocyclyl; and
each R^{4a} is independently H, CN, or C₁-C₆ alkyl.

In some embodiments, R³ is C₁-C₄ alkyl substituted with one or more R^{3a}, wherein:
each R^{3a} is independently OR^{4a}, C₆-C₁₀ aryl, or 3- to 12-membered heterocyclyl; and
each R^{4a} is independently H, CN, or C₁-C₆ alkyl.
   In Some Embodiments, R³ is C₁-C₃ Alkyl Substituted with One or More R^{3a}, Wherein:
each R^{3a} is independently OR^{4a}, C₆-C₁₀ aryl, or 3- to 12-membered heterocyclyl; and
each R^{4a} is independently H, CN, or C₁-C₆ alkyl.
   In Some Embodiments, R³ is C₁-C₂ Alkyl Substituted with One or More R^{3a}, Wherein:
each R^{3a} is independently OR⁴, C₆-C₁₀ aryl, or 3- to 12-membered heterocyclyl; and
each R^{4a} is independently H, CN, or C₁-C₆ alkyl.
   In Some Embodiments, R³ is Methyl Substituted with One or More R^{3a}, Wherein:
each R^{3a} is independently OR^{4a}, C₆-C₁₀ aryl, or 3- to 12-membered heterocyclyl; and
each R¹ is independently H, CN, or C₁-C₆ alkyl.
   In Some Embodiments, R³ is Ethyl Substituted with One or More R^{3a}, Wherein:
each R^{3a} is independently OR^{4a}, C₆-C₁₀ aryl, or 3- to 12-membered heterocyclyl; and
each R¹ is independently H, CN, or C₁-C₆ alkyl.
   In Some Embodiments, R³ is Propyl Substituted with One or More R^{3a}, Wherein:
each R^{3a} is independently OR^{4a}, C₆-C₁₀ aryl, or 3- to 12-membered heterocyclyl; and
each R^{4a} is independently H, CN, or C₁-C₆ alkyl.

In some embodiments, R³ is methyl substituted with one or more R^{3a}, wherein each R^{3a} is independently C₆-C₁₀ aryl.

In some embodiments, R³ is methyl substituted with one phenyl.

In some embodiments, R³ is ethyl substituted with one or more R^{3a}, wherein each R^{3a} is independently C₆-C₁₀ aryl.

In some embodiments, R³ is propyl substituted with one or more R^{3a}, wherein each R^{3a} is independently C₆-C₁₀ aryl.

In some embodiments, R³ is isopropyl substituted with one or more R^{3a}, wherein each R^{3a} is independently C₆-C₁₀ aryl.

In some embodiments, R³ is butyl substituted with one or more R^{3a}, wherein each R^{3a} is independently C₆-C₁₀ aryl.

In some embodiments, R³ is tert-butyl substituted with one or more R^{3a}, wherein each R^{3a} is independently C₆-C₁₀ aryl.

In some embodiments, R³ is pentyl substituted with one or more R^{3a}, wherein each R^{3′} is independently C₆-C₁₀ aryl.

In some embodiments, R³ is hexyl substituted with one or more R^{3a}, wherein each R^{3a} is independently C₆-C₁₀ aryl.

In some embodiments, R³ is C₁-C₆ alkyl substituted with one or more R^{3a}, wherein each R^{3a} is independently 3- to 12-membered heterocyclyl.

In some embodiments, R³ is C₁-C₈ alkyl substituted with one or more R^{3a}, wherein each R^{3a} is independently 3- to 12-membered heterocyclyl.

In some embodiments, R³ is C₁-C₄ alkyl substituted with one or more R^{3a}, wherein each R^{3a} is independently 3- to 12-membered heterocyclyl.

In some embodiments, R³ is C₁-C₃ alkyl substituted with one or more R^{3a}, wherein each R^{3a} is independently 3- to 12-membered heterocyclyl.

In some embodiments, R³ is C₁-C₂ alkyl substituted with one or more R^{3a}, wherein each R^{3a} is independently 3- to 12-membered heterocyclyl.

In some embodiments, R³ is methyl substituted with one or more R^{3a}, wherein each R^{3a} is independently 3- to 12-membered heterocyclyl.

In some embodiments, R³ is ethyl substituted with one or more R^{3a}, wherein each R^{3a} is independently 3- to 12-membered heterocyclyl.

In some embodiments, R³ is propyl substituted with one or more R^{3a}, wherein each R^{3a} is independently 3- to 12-membered heterocyclyl.

In some embodiments, R³ is

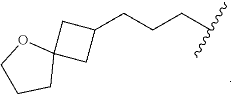 [SMILES: *CCCC1CC2(CCCO2)C1]

In some embodiments, R³ is isopropyl substituted with one or more R^{3a}, wherein each R^{3a} is independently 3- to 12-membered heterocyclyl.

In some embodiments, R³ is butyl substituted with one or more R^{3a}, wherein each R^{3a} is independently 3- to 12-membered heterocyclyl.

In some embodiments, R³ is tert-butyl substituted with one or more R^{3a}, wherein each R^{3a} is independently 3- to 12-membered heterocyclyl.

In some embodiments, R³ is pentyl substituted with one or more R^{3a}, wherein each R^{3a} is independently 3- to 12-membered heterocyclyl.

In some embodiments, R³ is hexyl substituted with one or more R^{3a}, wherein each R^{3a} is independently 3- to 12-membered heterocyclyl.

In some embodiments, R³ forms a 3- to 12-membered heterocyclyl with the carbon atom next to the nitrogen atom to which it is connected.

In some embodiments,

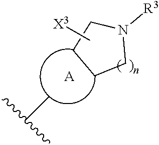

is

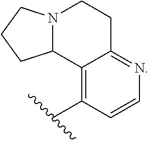 [SMILES: *c1ccnc2c1C1CCCN1CC2]

In some embodiments, n is 1.

In some embodiments, n is 2.

In some embodiments, n is 3.

In some embodiments, W is CH, Y is N, Z is O, and Ring A is

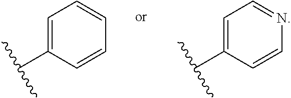 [SMILES: *c1ccccc1 ; "or" ; *c1ccncc1]

In some embodiments, W is CH; Y is N; Z is O; Ring A is

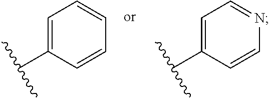 [SMILES: *c1ccccc1 ; "or" ; *c1ccncc1]

R² is isopropyl; and R¹ is

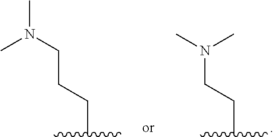

In some embodiments, W is CH; Y is N; Z is O; Ring A is

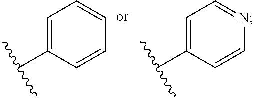 [SMILES: *c1ccccc1 ; "or" ; *c1ccncc1]

R² is isopropyl; and R¹ is

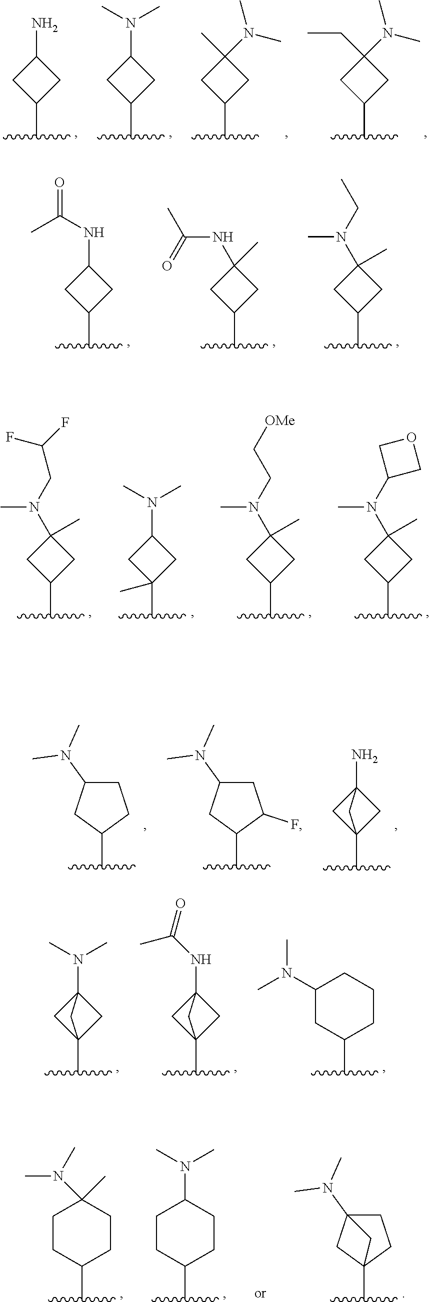

In some embodiments, W is CH; Y is N; Z is O; Ring A is

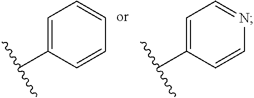 [SMILES: *c1ccccc1 ; "or" ; *c1ccncc1]

R² is isopropyl; and R¹ is

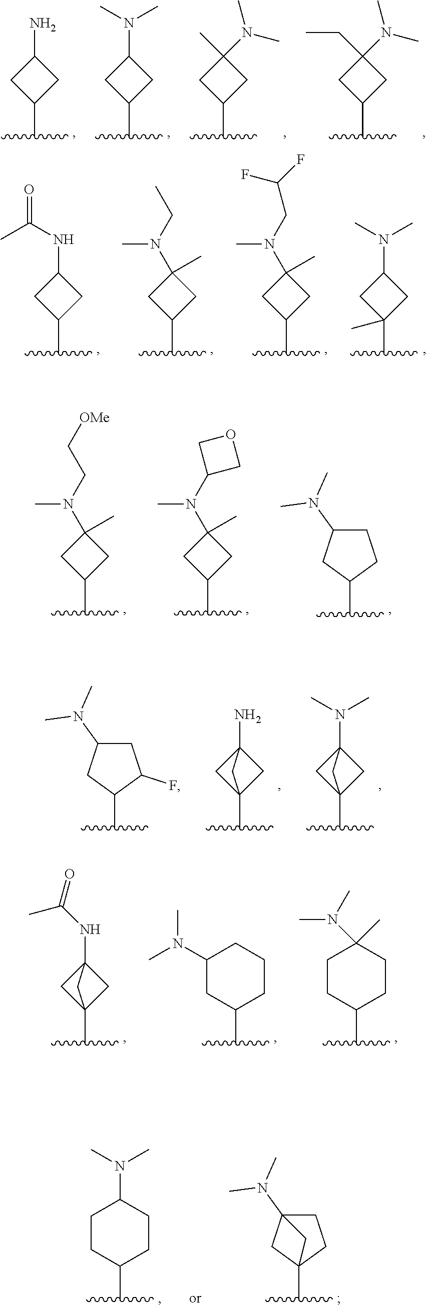

In some embodiments, W is CH; Y is N; Z is O; Ring A is

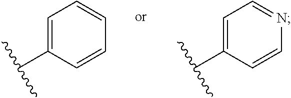 [SMILES: *c1ccccc1 ; "or" ; *c1ccncc1]

R² is isopropyl; and R¹ is

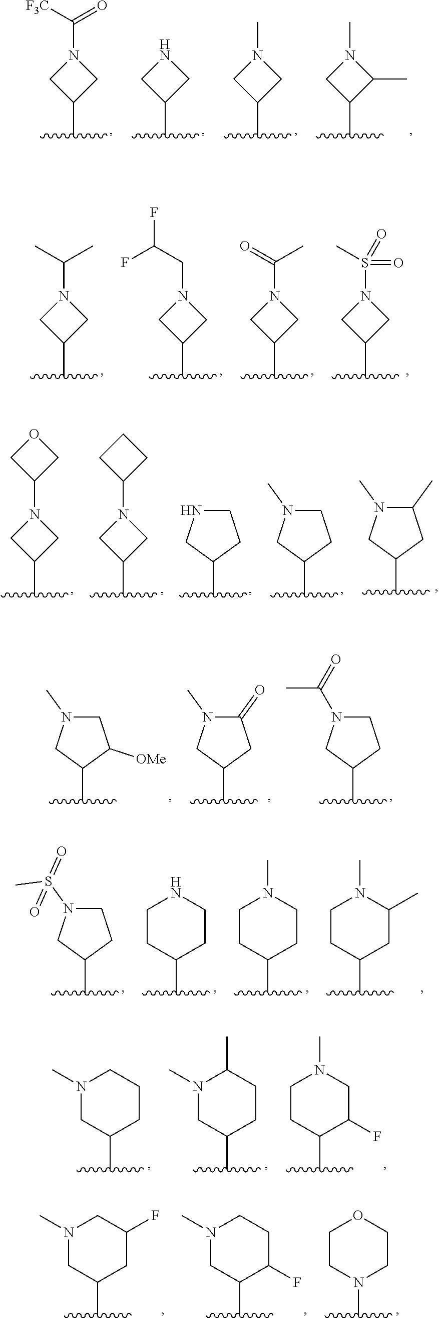

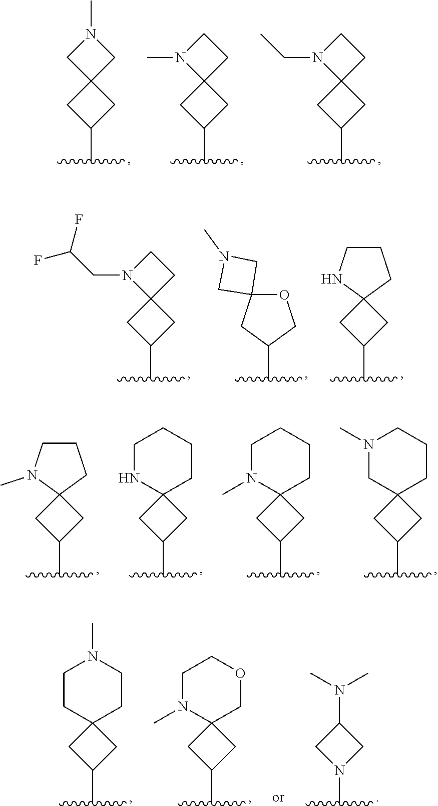

In some embodiments, W is CH, Y is N; Z is O, Ring A is

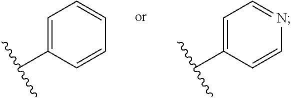 [SMILES: *c1ccccc1 ; "or" ; *c1ccncc1]

R² is isopropyl; and R¹ is

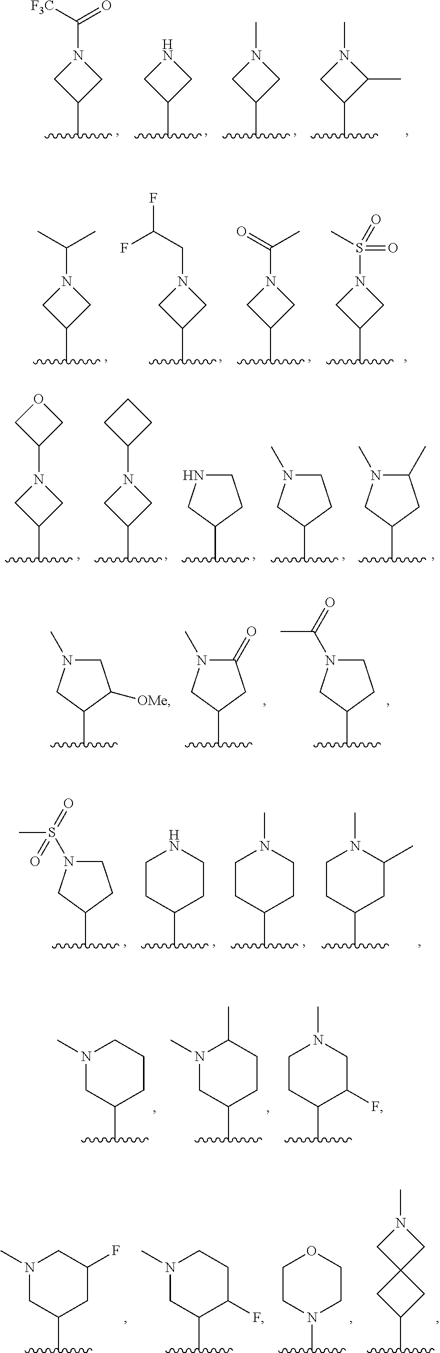

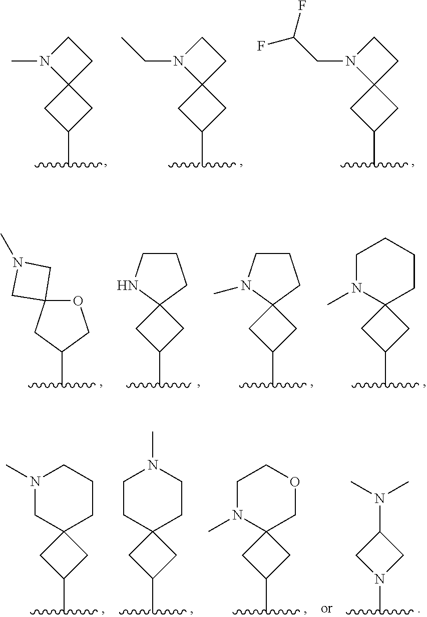

In some embodiments, W is CH; Y is N; Z is O; Ring A is

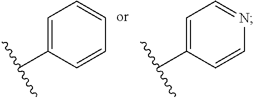 [SMILES: *c1ccccc1 ; "or" ; *c1ccncc1]

R² is isopropyl; and R¹ is

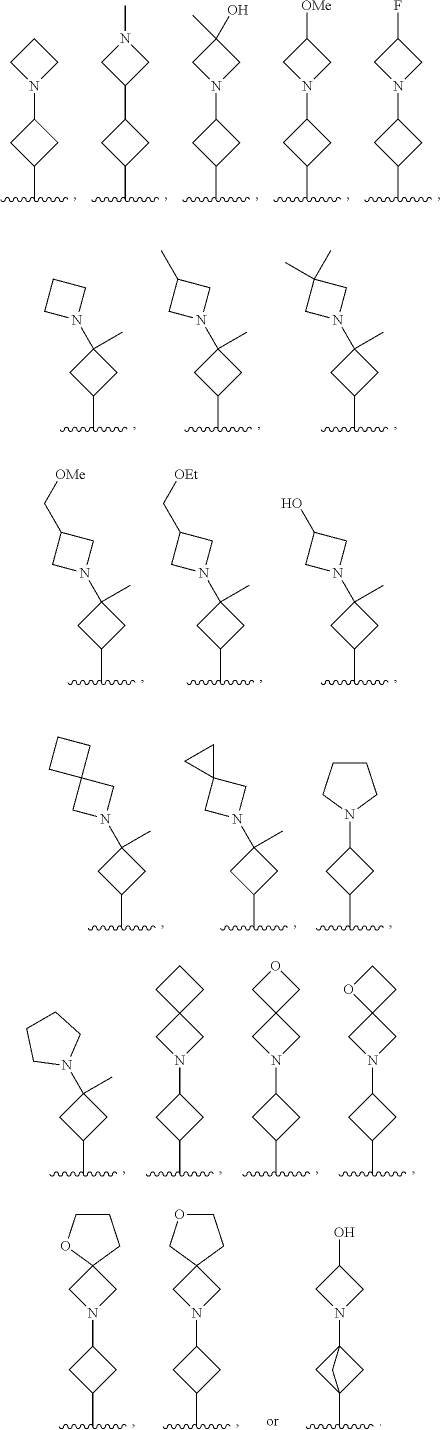

In some embodiments, W is CH; Y is N; Z is O; Ring A is

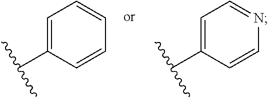 [SMILES: *c1ccccc1 ; "or" ; *c1ccncc1]

and X³ is H, methyl, or ethyl.

In some embodiments, W is CH; Y is N; Z is O; Ring A is

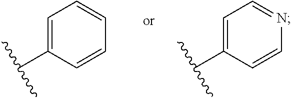 [SMILES: *c1ccccc1 ; *c1ccncc1]

X³ is H, methyl, or ethyl; R² is isopropyl; and R¹ is

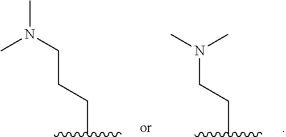

In some embodiments, W is CH; Y is N; Z is O; Ring A is

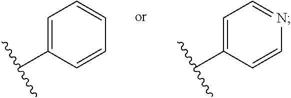 [SMILES: *c1ccccc1 ; "or" ; *c1ccncc1]

X³ is H, methyl, or ethyl; R² is isopropyl; and R¹ is

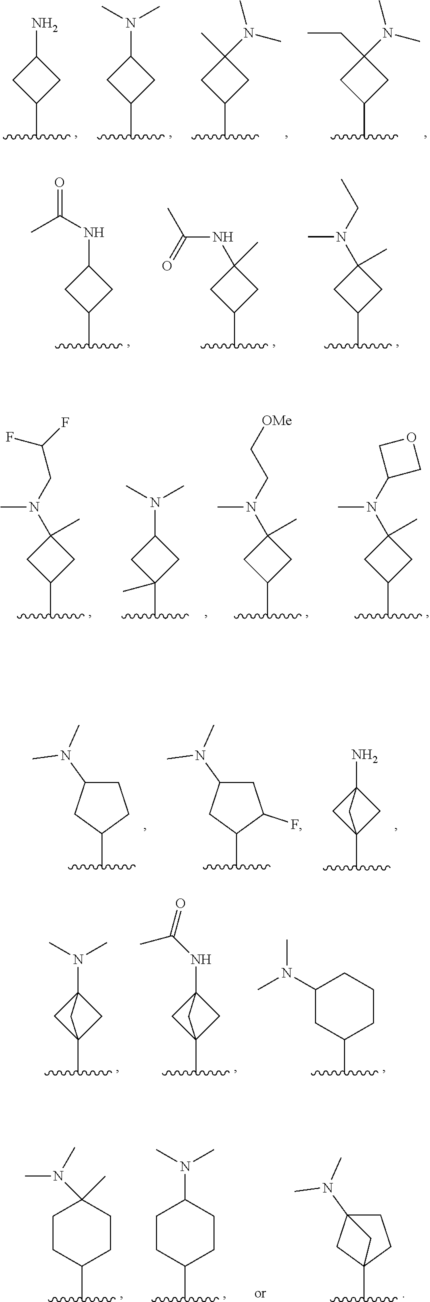

In some embodiments, W is CH; Y is N; Z is O; Ring A is

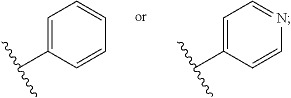 [SMILES: *c1ccccc1 ; "or" ; *c1ccncc1]

X³ is H, methyl, or ethyl; R² is isopropyl; and R¹ is

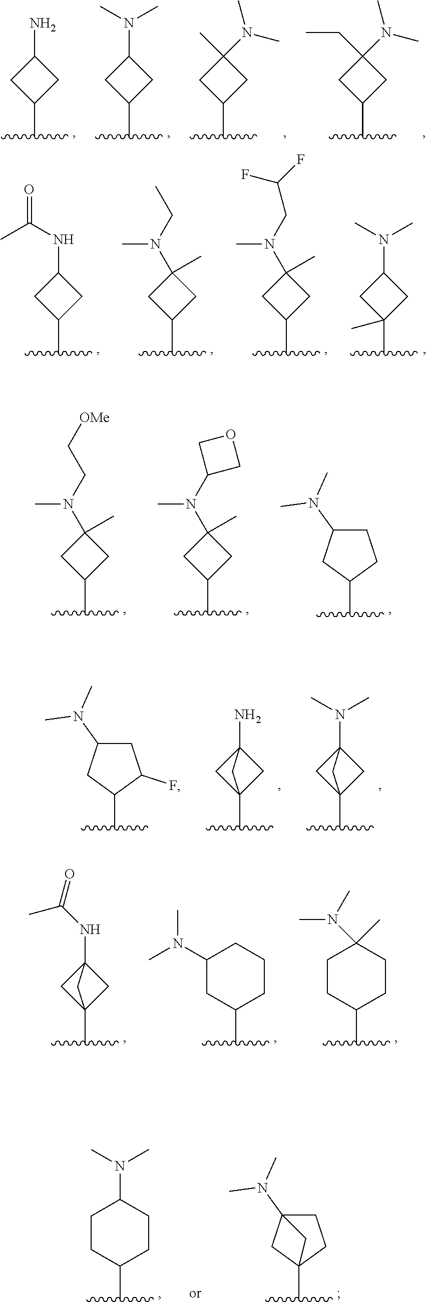

In some embodiments, W is CH; Y is N, Z is O; Ring A is

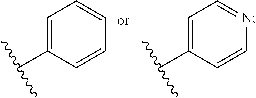 [SMILES: *c1ccccc1 ; "or" ; *c1ccncc1]

X³ is H, methyl, or ethyl; R² is isopropyl; and R¹ is

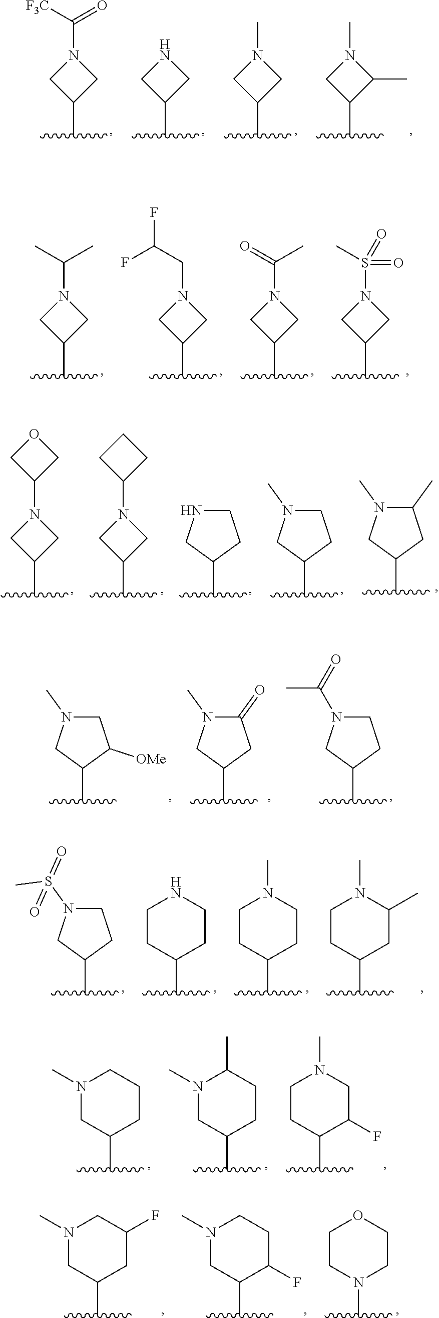

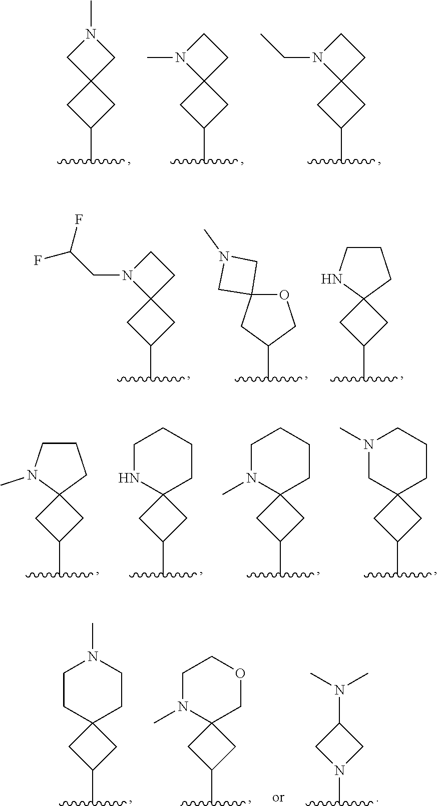

In some embodiments, W is CH; Y is N; Z is O; Ring A is

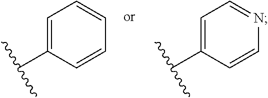 [SMILES: *c1ccccc1 ; "or" ; *c1ccncc1]

X³ is H, methyl, or ethyl; R² is isopropyl; and R¹ is

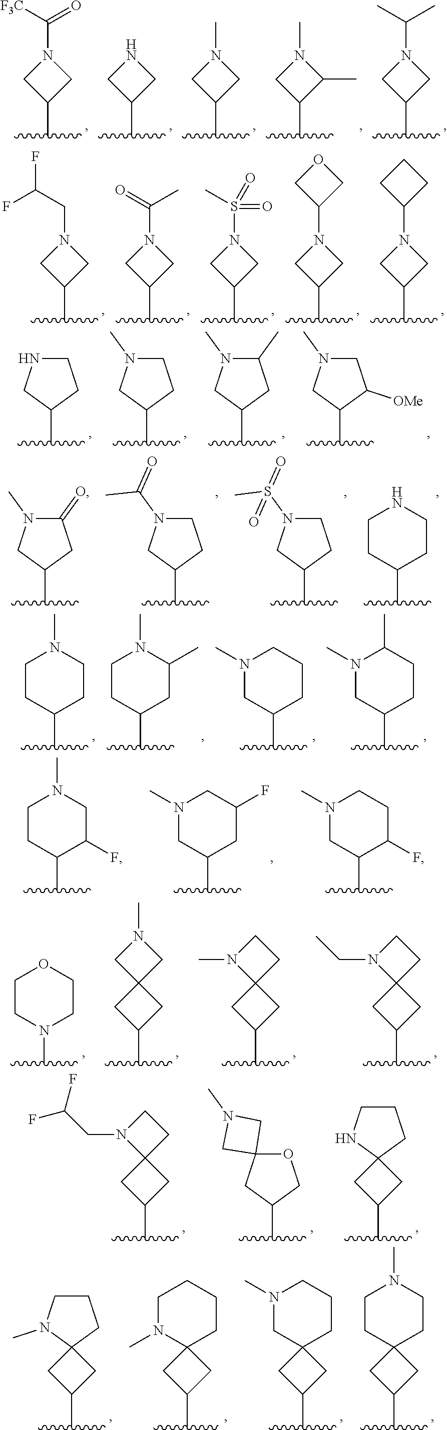

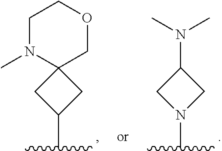

In some embodiments, W is CH; Y is N; Z is O; Ring A is

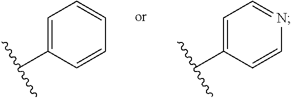 [SMILES: *c1ccccc1 ; "or" ; *c1ccncc1]

X³ is H, methyl, or ethyl; R² is isopropyl; and R¹ is

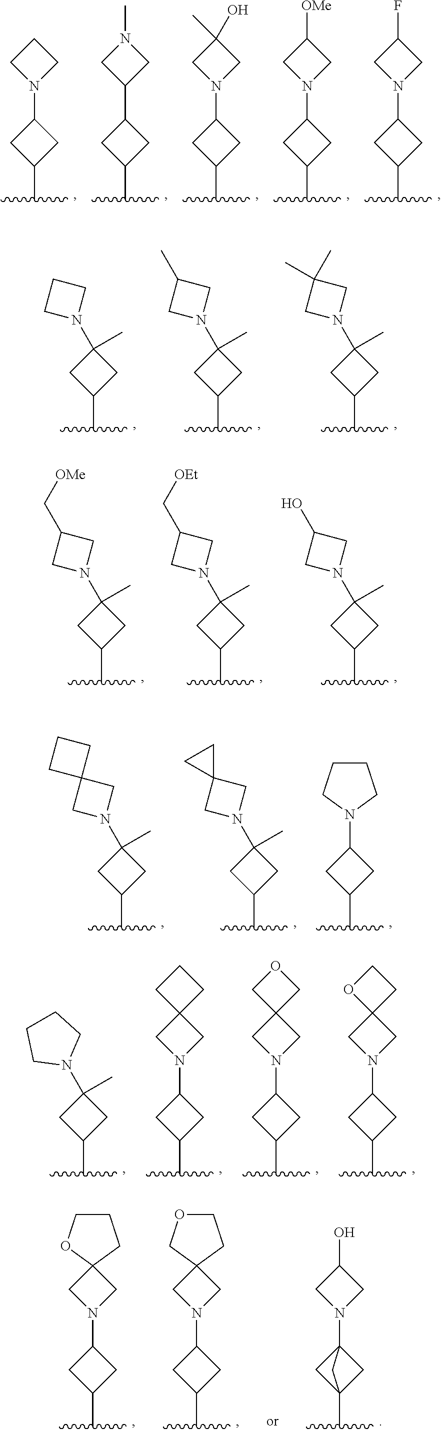

In some embodiments, W is CH; Y is N; Z is O; Ring A is

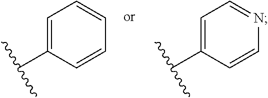 [SMILES: *c1ccccc1 ; "or" ; *c1ccncc1]

R³ is H, methyl, ethyl, isopropyl, tert-butyl,

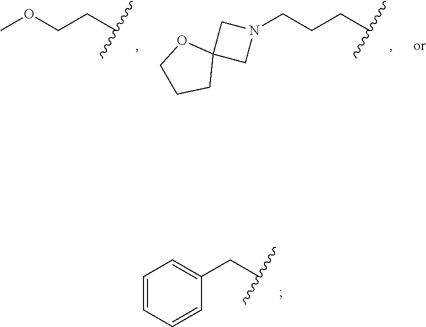

and X³ is H, methyl, or ethyl.

In some embodiments, W is CH; Y is N; Z is O; Ring A is

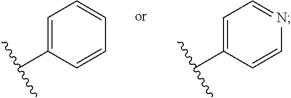 [SMILES: *c1ccccc1 ; "or" ; *c1ccncc1]

R³ is H, methyl, ethyl, isopropyl, tert-butyl,

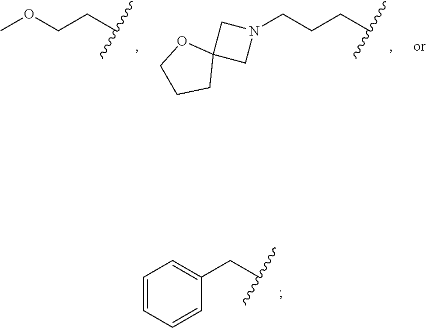

X³ is H, methyl, or ethyl; R² is isopropyl; and R¹ is

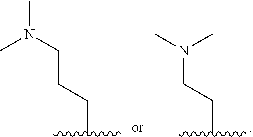

In some embodiments, W is CH; Y is N; Z is O; Ring A is

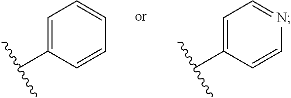 [SMILES: *c1ccccc1 ; "or" ; *c1ccncc1]

R³ is H, methyl, ethyl, isopropyl, tert-butyl,

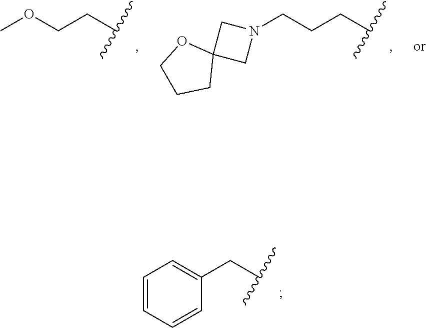

X³ is H, methyl, or ethyl; R² is isopropyl; and R¹ is

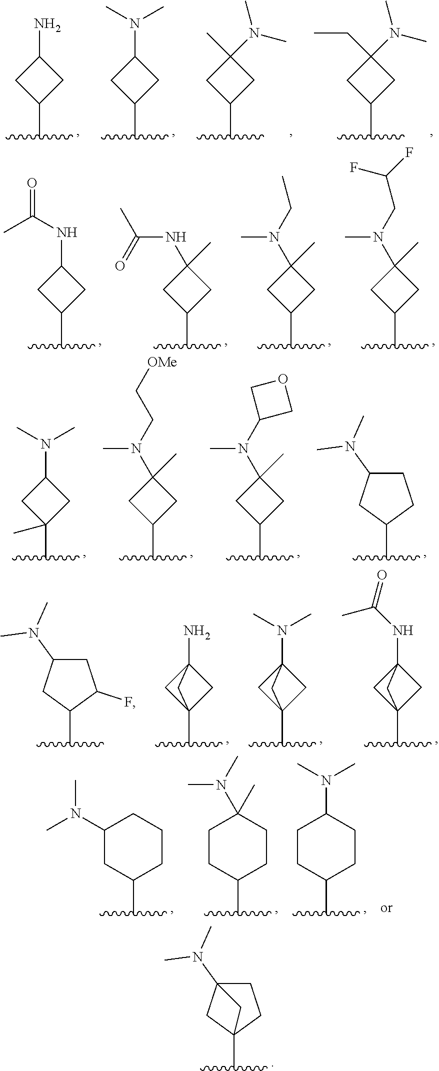

In some embodiments, W is CH, Y is N; Z is O; Ring A is

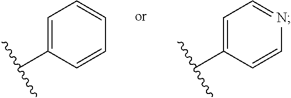 [SMILES: *c1ccccc1 ; "or" ; *c1ccncc1]

R³ is H, methyl, ethyl, isopropyl, tert-butyl,

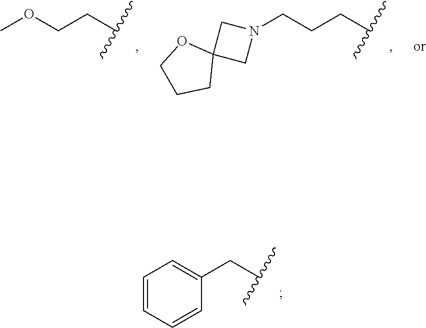

X³ is H, methyl, or ethyl; R² is isopropyl; and R¹ is

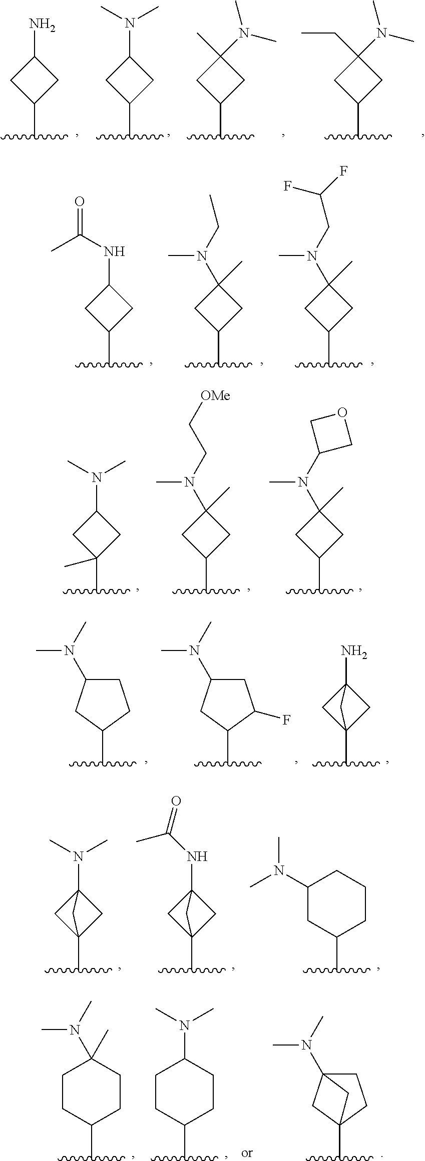

In same embodiments, W is CH; Y is N; Z is O; Ring A is

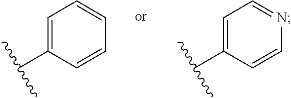 [SMILES: *c1ccccc1 ; "or" ; *c1ccncc1]

R³ is H, methyl, ethyl, isopropyl, tert-butyl,

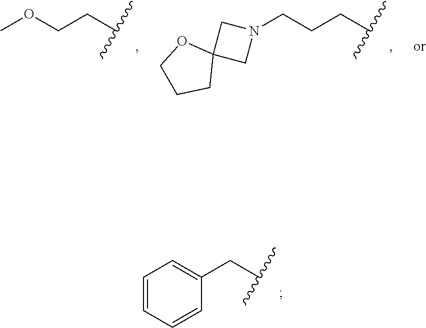

X³ is H, methyl, or ethyl; R² is isopropyl; and R¹ is

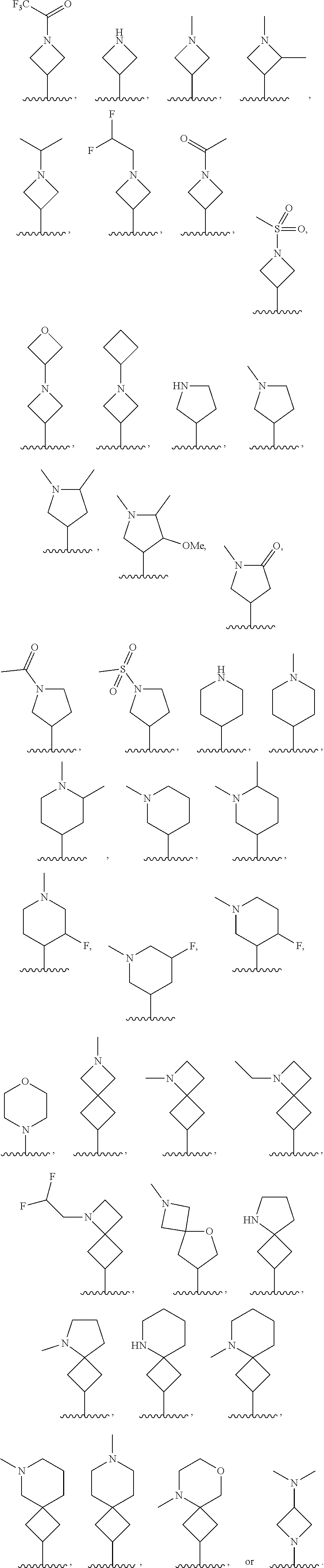

In some embodiments, W is CH; Y is N; Z is O; Ring A is

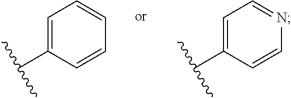 [SMILES: *c1ccccc1 ; "or" ; *c1ccncc1]

R³ is H, methyl, ethyl, isopropyl, tert-butyl,

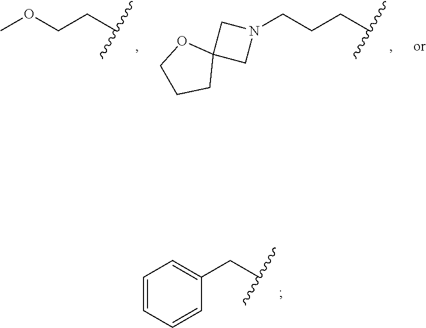

X³ is H, methyl, or ethyl; R² is isopropyl; and R¹ is

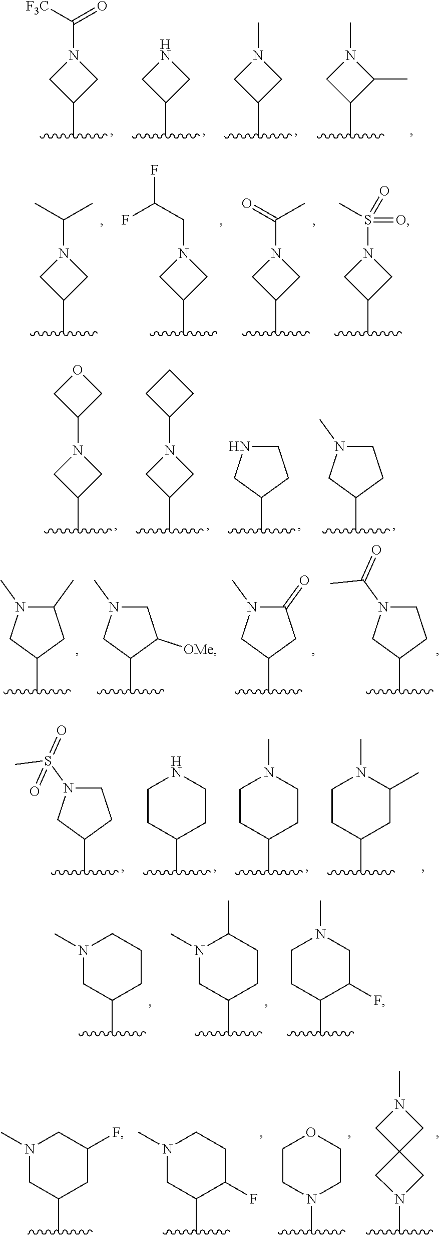

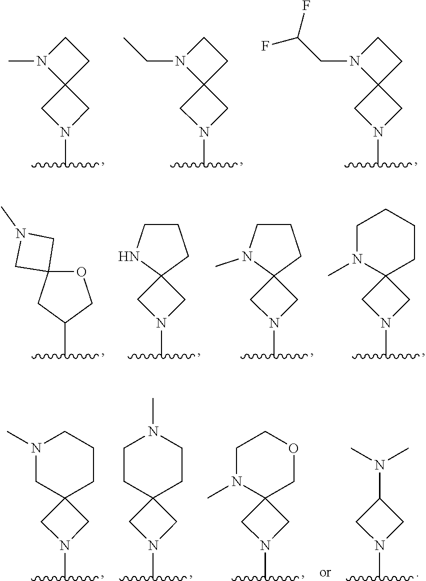

In some embodiments, W is CH; Y is N; Z is O; Ring A is

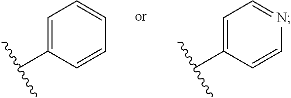 [SMILES: *c1ccccc1 ; "or" ; *c1ccncc1]

R³ is H, methyl, ethyl, isopropyl, tert-butyl,

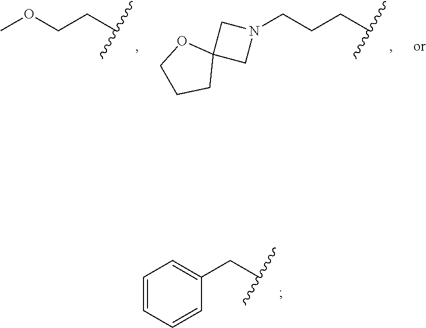

X³ is H, methyl, or ethyl; R² is isopropyl; and R¹ is

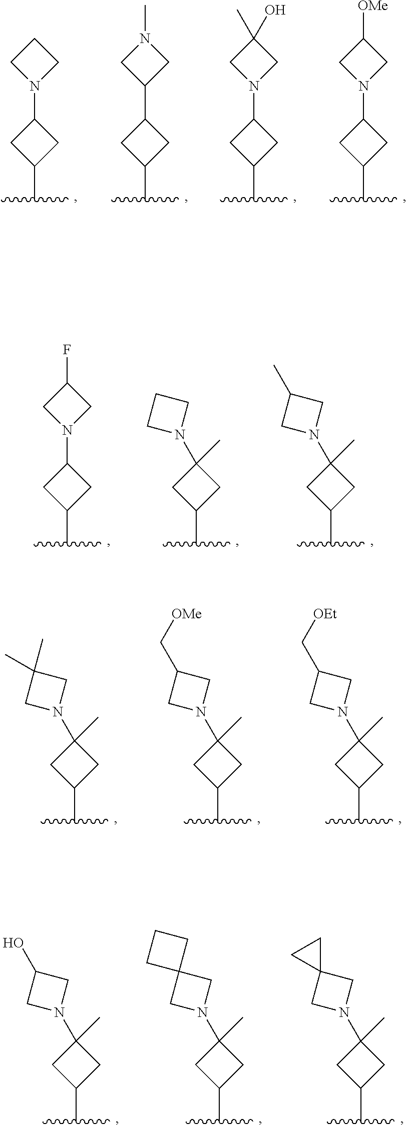

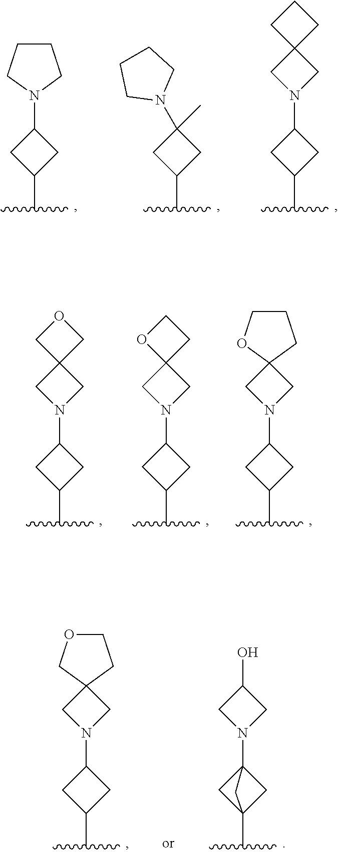

In some embodiments, W is CH, Y is N, Z is O, and

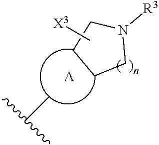

is

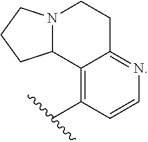 [SMILES: *c1ccnc2c1C1CCCN1CC2]

In some embodiments, W is CH; Y is N; Z is O;

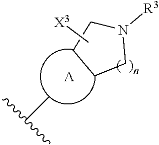

is

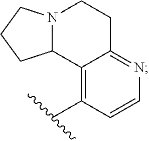 [SMILES: *c1ccnc2c1C1CCCN1CC2]

R² is isopropyl; and R¹ is

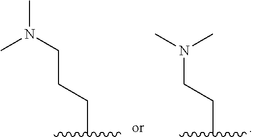

In some embodiments, W is CH; Y is N; Z is O;

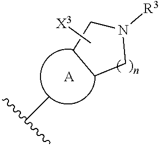

is

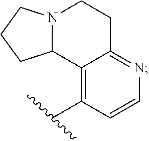 [SMILES: *c1ccnc2c1C1CCCN1CC2]

R² is isopropyl; and R¹ is

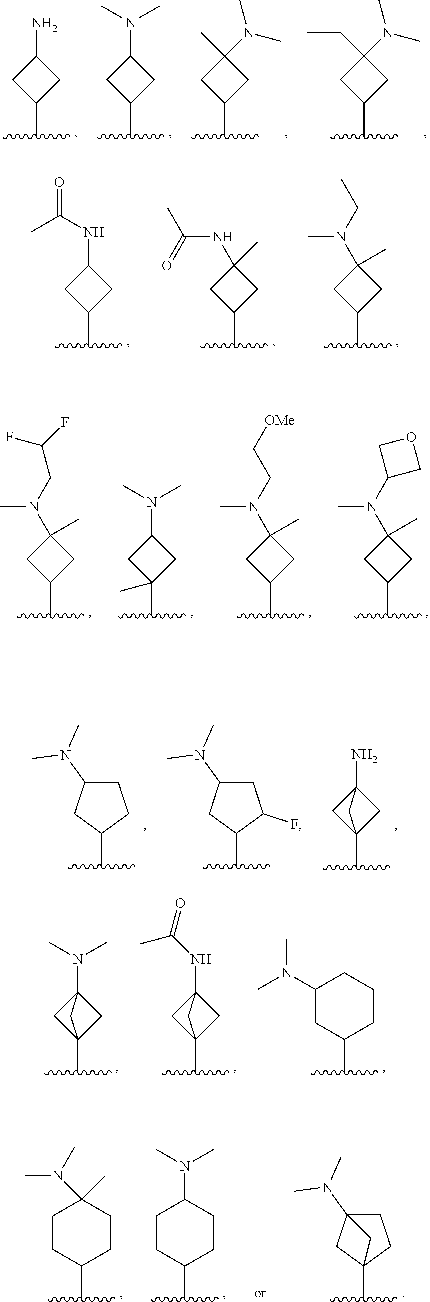

In some embodiments, W is CH; Y is N; Z is O;

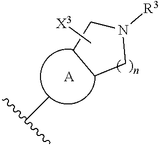

is

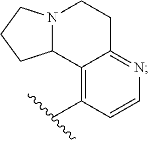 [SMILES: *c1ccnc2c1C1CCCN1CC2]

R² is isopropyl; and R¹ is

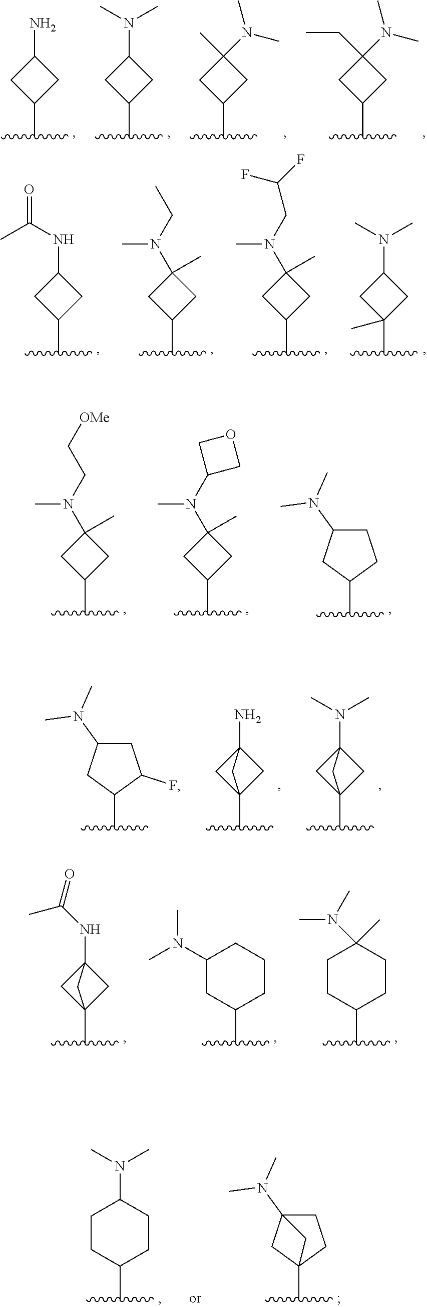

In some embodiments, W is CH; Y is N; Z is O;

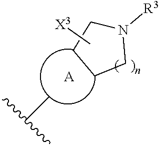

is

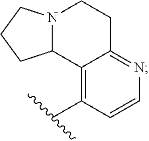 [SMILES: *c1ccnc2c1C1CCCN1CC2]

R² is isopropyl; and R¹ is

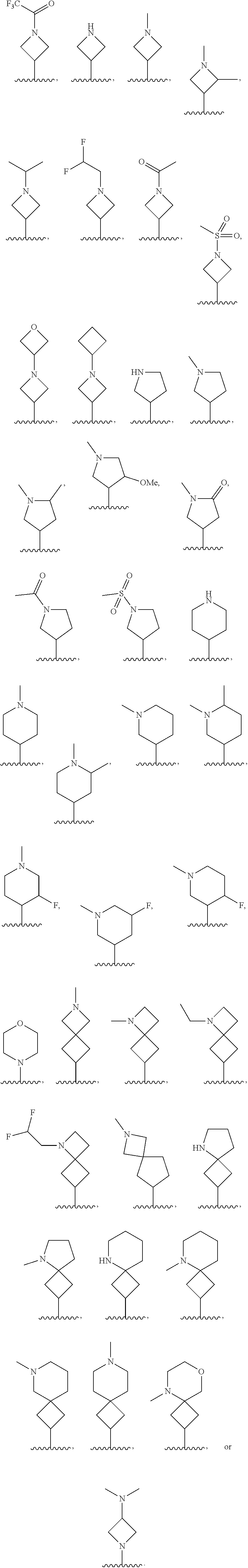

In some embodiments, W is CH; Y is N; Z is O;

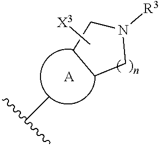

is

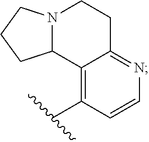 [SMILES: *c1ccnc2c1C1CCCN1CC2]

R² is isopropyl; and R¹ is

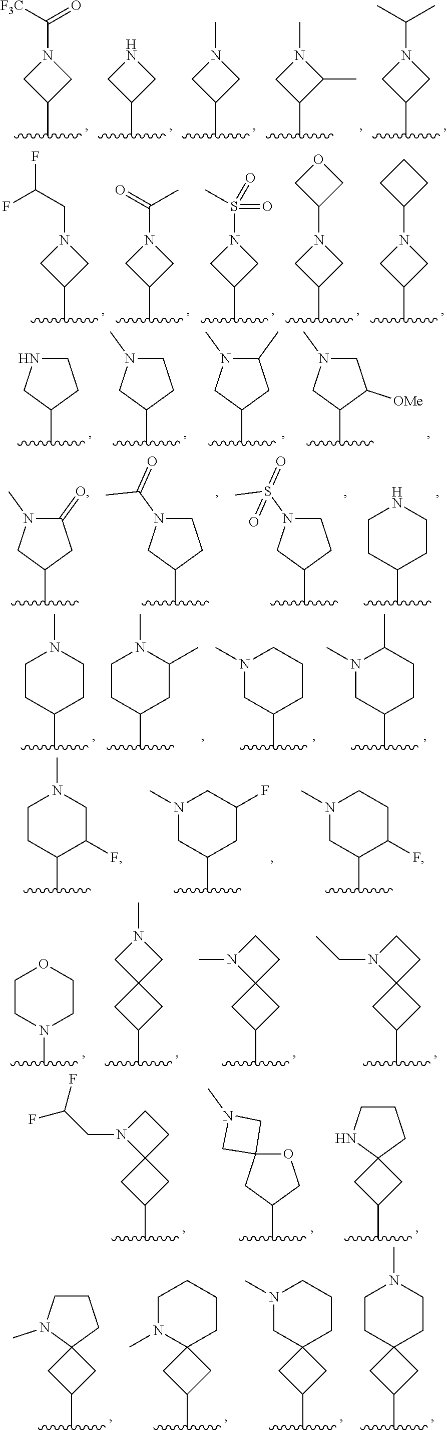

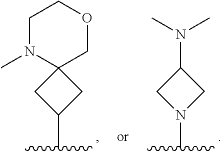

In some embodiments, W is CH; Y is N; Z is O;

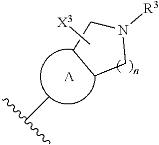

is

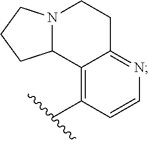 [SMILES: *c1ccnc2c1C1CCCN1CC2]

R² is isopropyl; and R¹ is

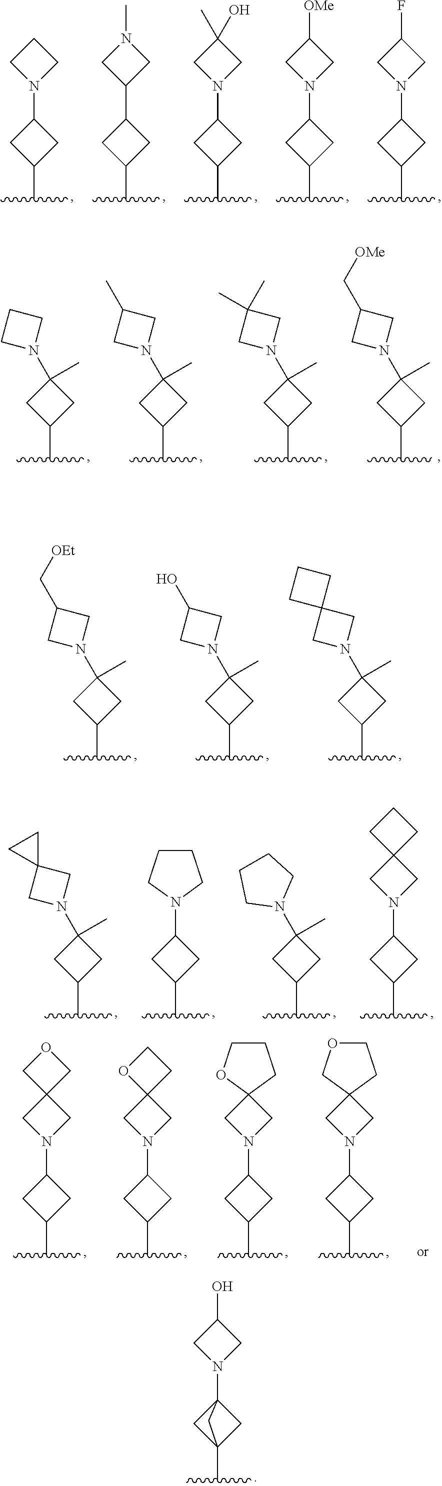

In some embodiments, W is CH, Y is N, Z is O, Ring A is

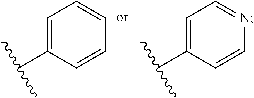 [SMILES: *c1ccccc1 ; "or" ; *c1ccncc1]

and n is 1, 2, or 3.

In some embodiments, W is CH; Y is N; Z is O; Ring A is

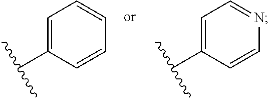 [SMILES: *c1ccccc1 ; "or" ; *c1ccncc1]

R² is isopropyl; R¹ is

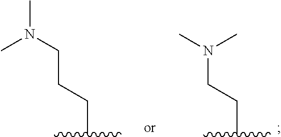

and n is 1, 2, or 3.

In some embodiments, W is CH; Y is N; Z is O; Ring A is

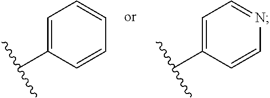 [SMILES: *c1ccccc1 ; "or" ; *c1ccncc1]

R² is isopropyl; R¹ is

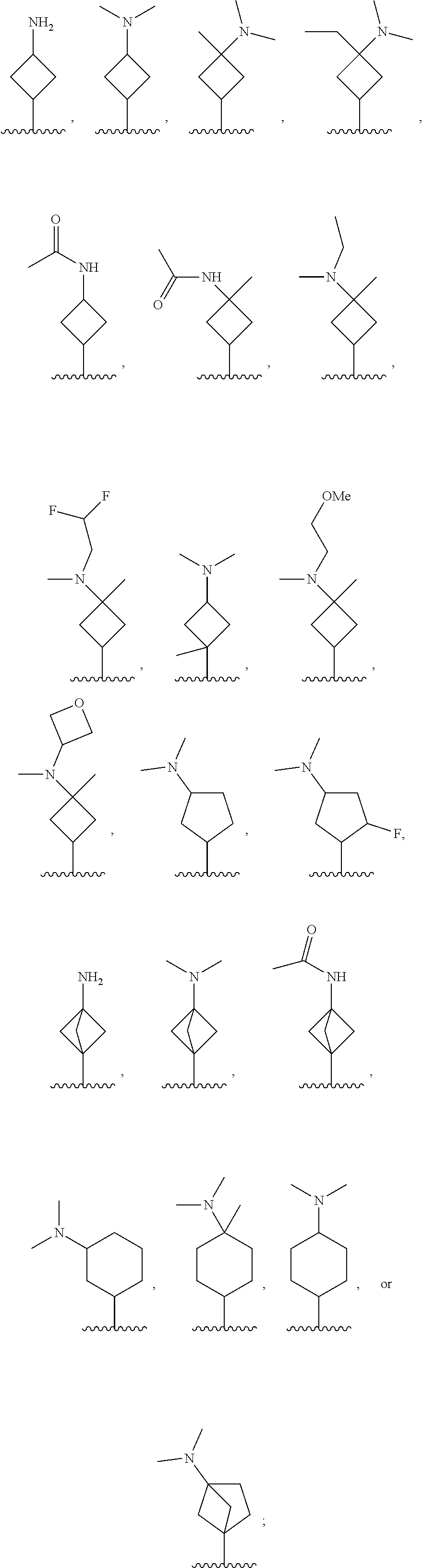

and n is 1, 2, or 3.

In some embodiments, W is CH; Y is N; Z is O; Ring A is

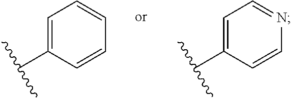 [SMILES: *c1ccccc1 ; "or" ; *c1ccncc1]

R² is isopropyl; R¹ is

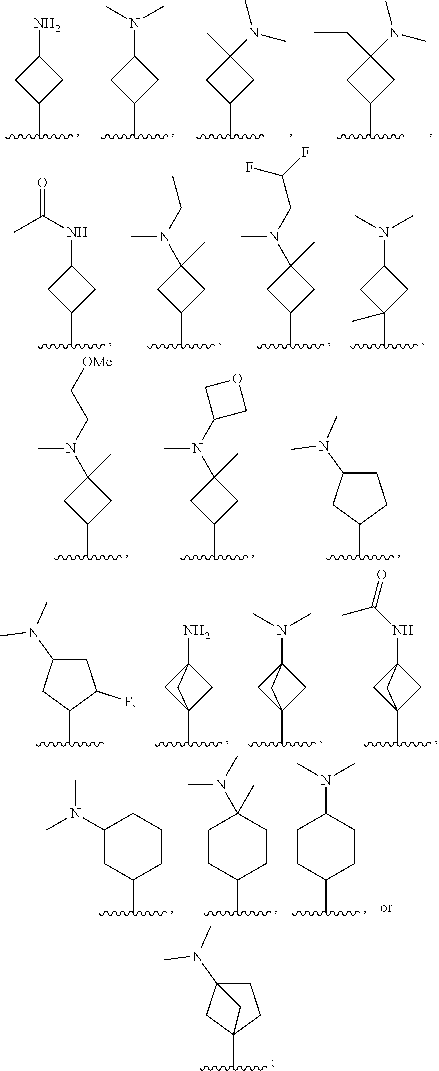

and n is 1, 2, or 3.

In some embodiments, W is CH; Y is N; Z is O; Ring A is

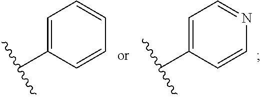 [SMILES: *c1ccccc1 ; *c1ccncc1]

R² is isopropyl, R¹ is

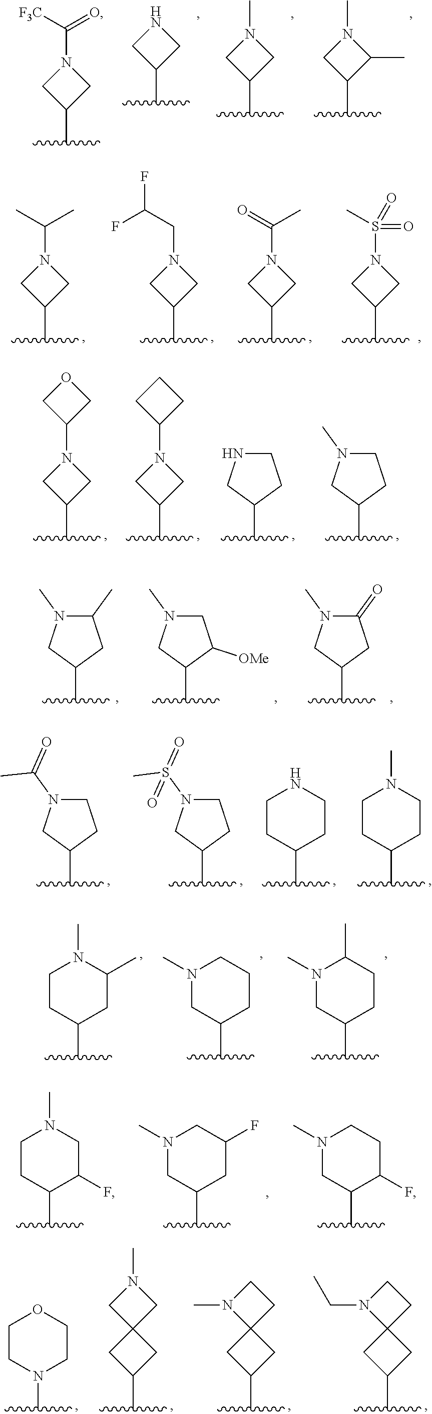

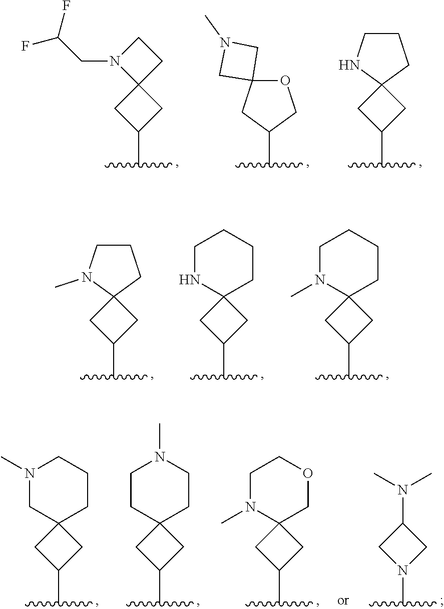

and n is 1, 2, or 3.

In some embodiments, W is CH; Y is N; Z is O; Ring A is

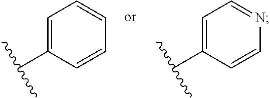 [SMILES: *c1ccccc1 ; "or" ; *c1ccncc1]

R² is isopropyl; R¹ is

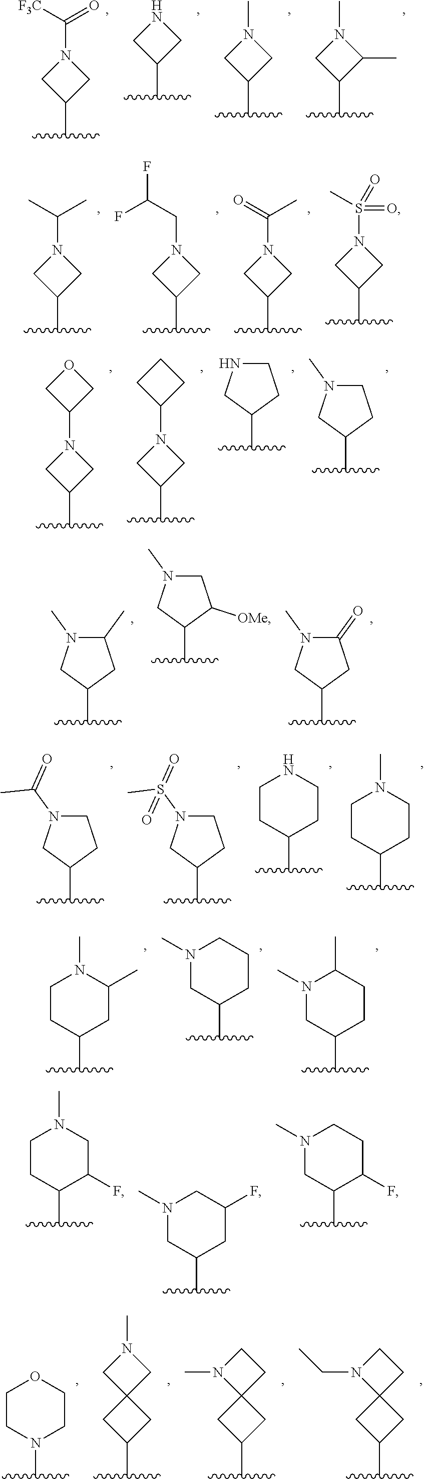

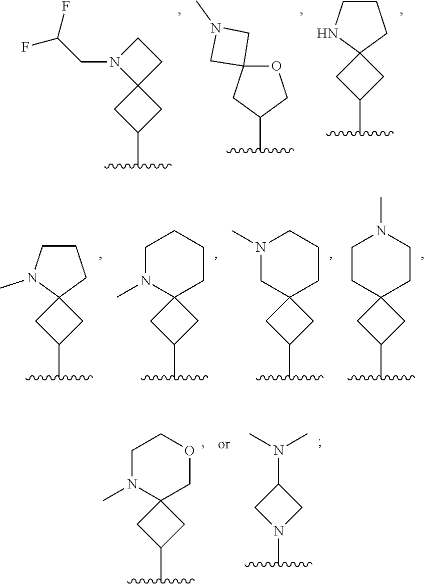

and n is 1, 2, or 3.

In some embodiments, W is CH; Y is N; Z is O; Ring A is

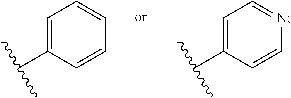 [SMILES: *c1ccccc1 ; "or" ; *c1ccncc1]

R² is isopropyl; R¹ is

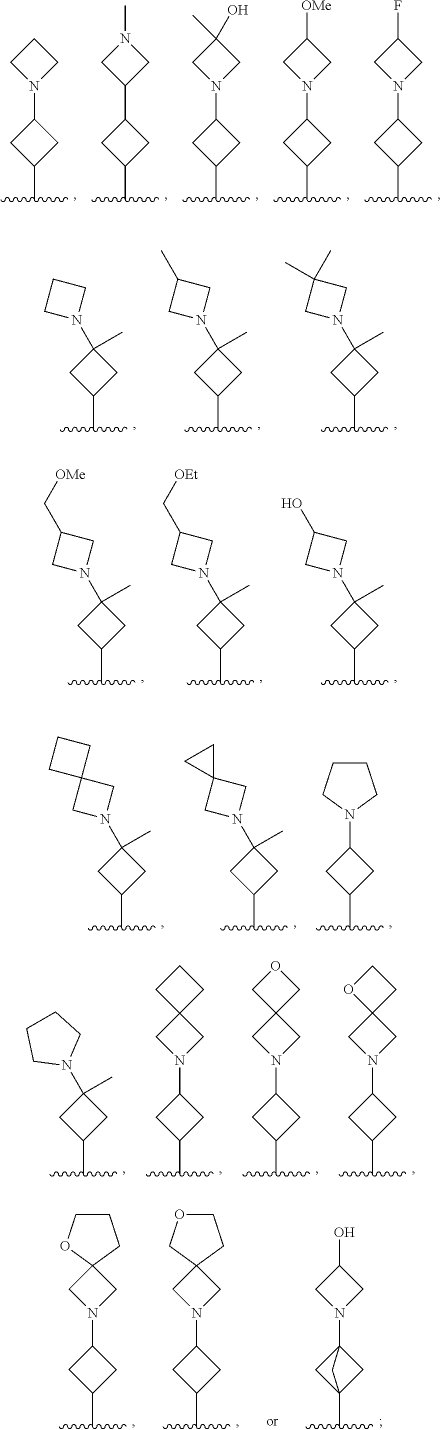

and n is 1, 2, or 3.

In some embodiments, W is CH; Y is N; Z is O; Ring A is

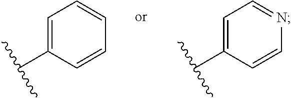 [SMILES: *c1ccccc1 ; "or" ; *c1ccncc1]

X³ is H, methyl, or ethyl; and n is 1, 2, or 3.

In some embodiments, W is CH; Y is N; Z is O; Ring A is

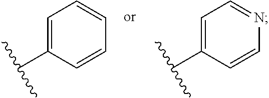 [SMILES: *c1ccccc1 ; "or" ; *c1ccncc1]

X³ is H, methyl, or ethyl; R² is isopropyl; R¹ is

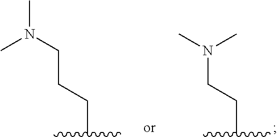

and n is 1, 2 or 3.

In some embodiments, W is CH; Y is N; Z is O; Ring A is

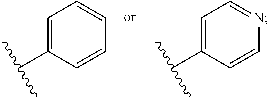 [SMILES: *c1ccccc1 ; "or" ; *c1ccncc1]

X³ is H, methyl, or ethyl; R² is isopropyl; R¹ is

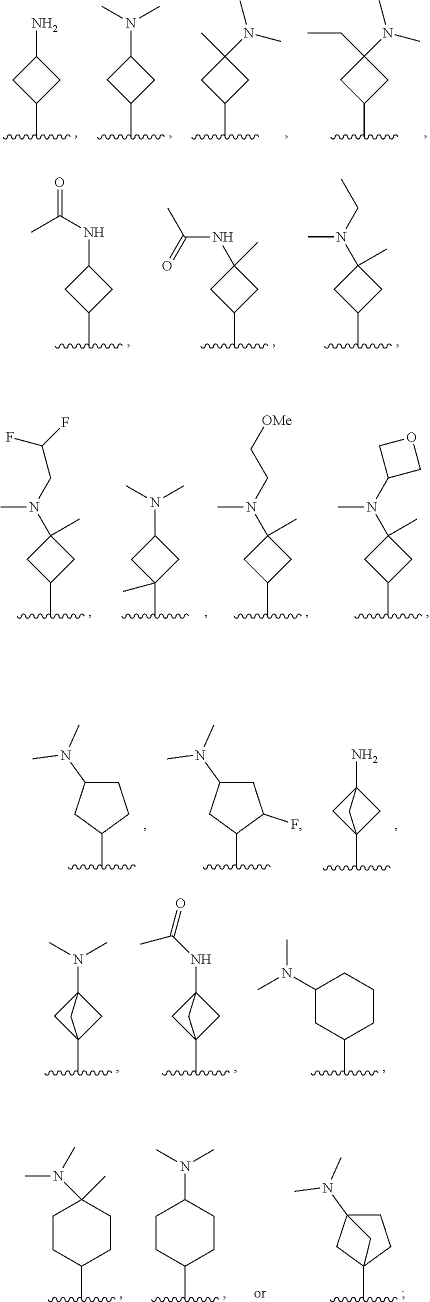

In some embodiments, W is CH; Y is N; Z is O; Ring A is

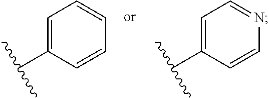 [SMILES: *c1ccccc1 ; "or" ; *c1ccncc1]

X³ is H, methyl, or ethyl; R² is isopropyl; R¹ is

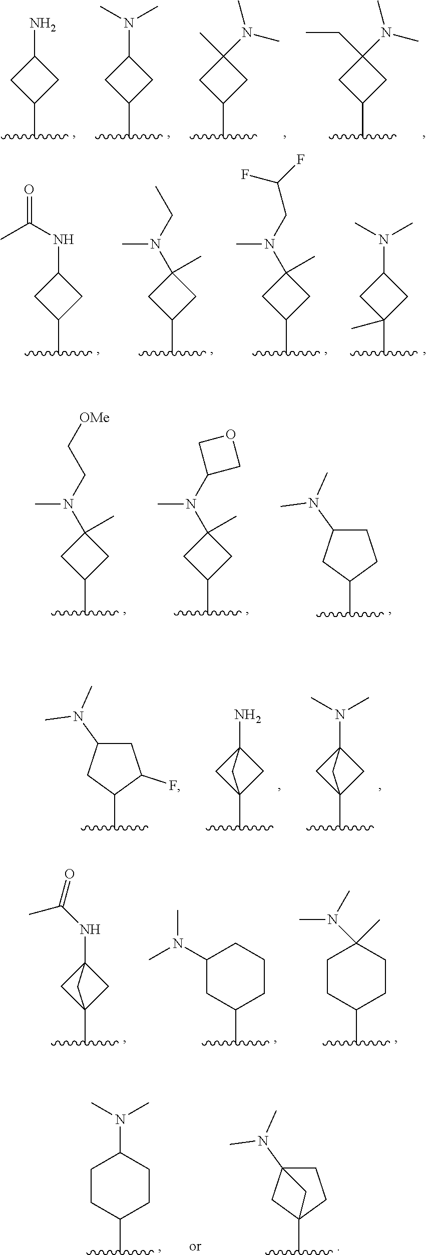

In some embodiments, W is CH; Y is N; Z is O; Ring A is

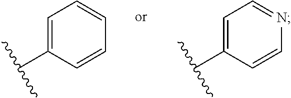 [SMILES: *c1ccccc1 ; "or" ; *c1ccncc1]

X³ is H, methyl, or ethyl; R² is isopropyl; R¹ is

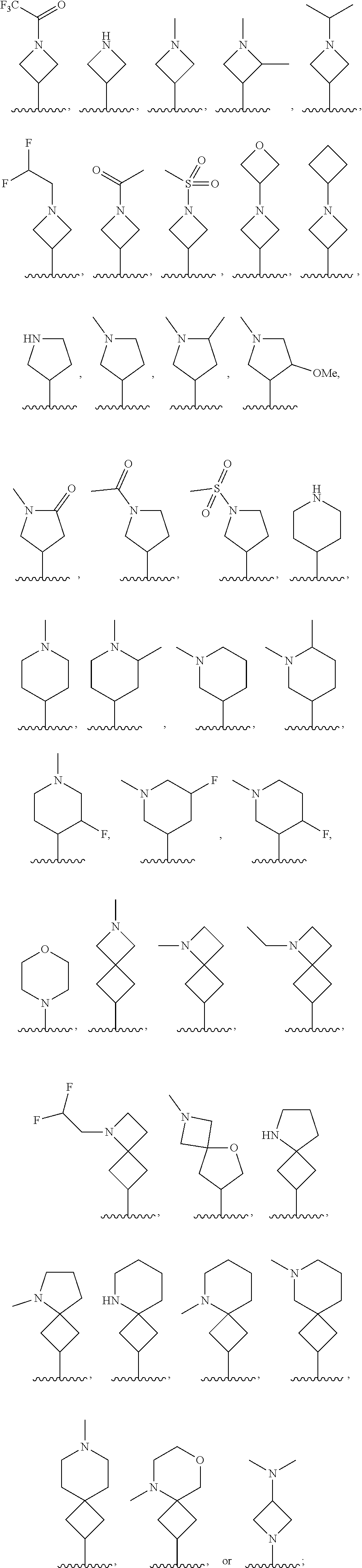

n is 1, 2, or 3.

In some embodiments, W is CH; Y is N; Z is O; Ring A is

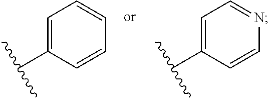 [SMILES: *c1ccccc1 ; "or" ; *c1ccncc1]

X³ is H, methyl, or ethyl; R² is isopropyl; R¹ is

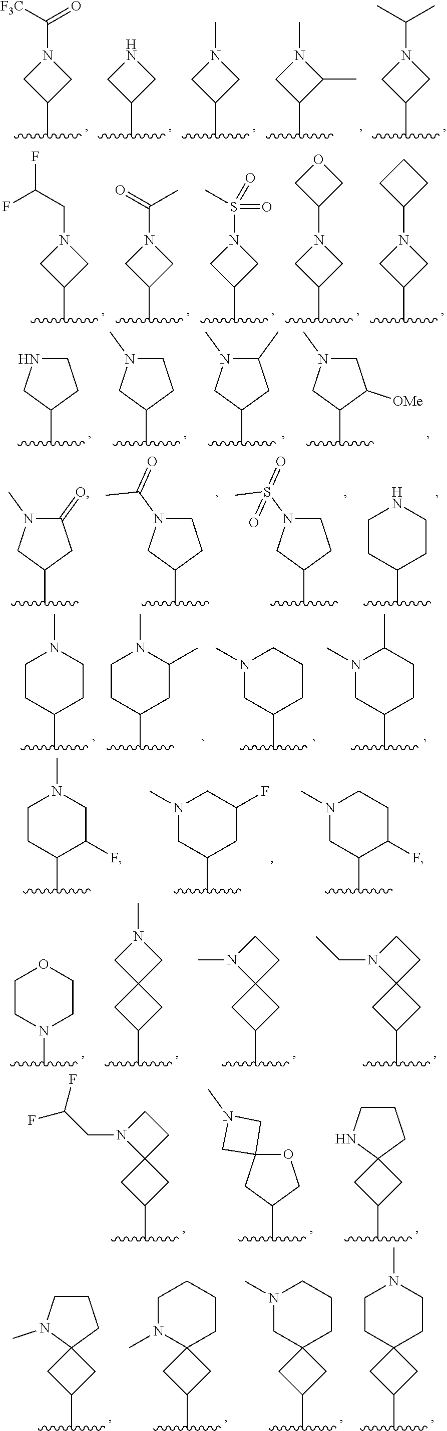

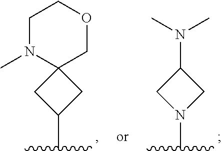

and n is 1, 2, or 3.

In some embodiments, W is CH; Y is N; Z is O; Ring A is

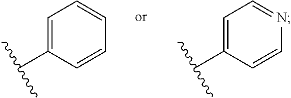 [SMILES: *c1ccccc1 ; "or" ; *c1ccncc1]

X³ is H, methyl, or ethyl; R² is isopropyl; R¹ is

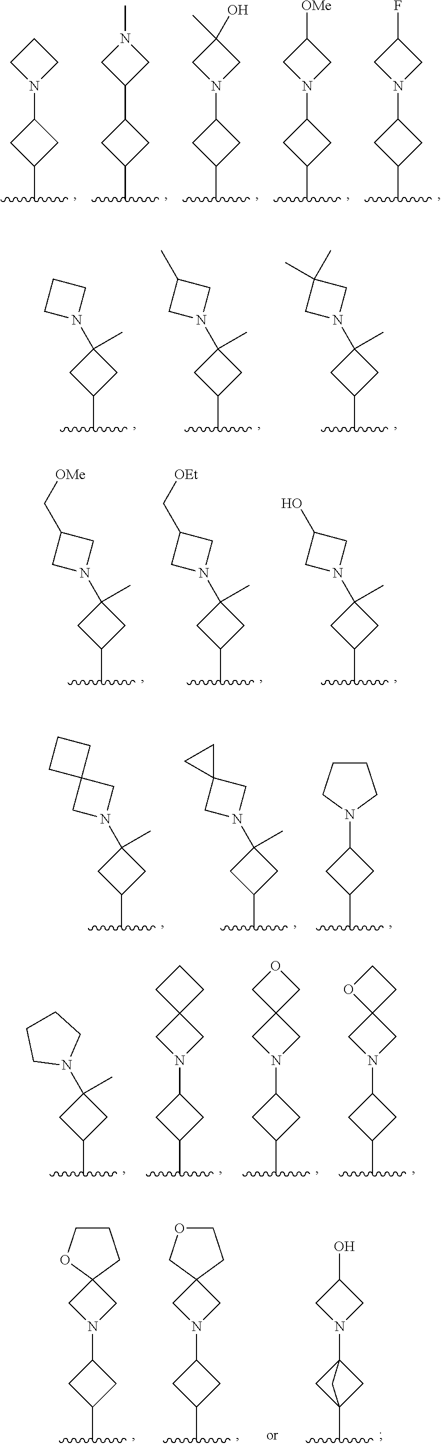

and n is 1, 2, or 3.

In some embodiments, W is CH; Y is N; Z is O; Ring A is

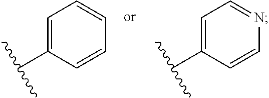 [SMILES: *c1ccccc1 ; "or" ; *c1ccncc1]

R³ is H, methyl, ethyl, isopropyl, tert-butyl,

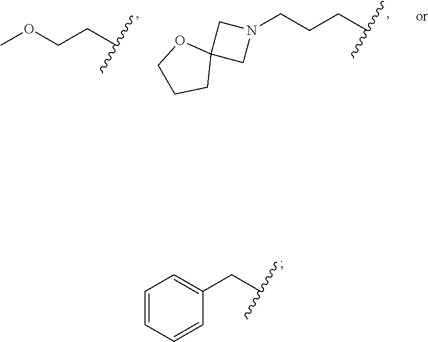

X³ is H, methyl, or ethyl; and n is 1, 2, or 3.

In some embodiments, W is CH; Y is N; Z is O; Ring A is

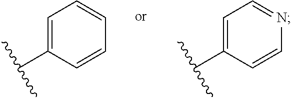 [SMILES: *c1ccccc1 ; "or" ; *c1ccncc1]

R³ is H, methyl, ethyl, isopropyl, tert-butyl,

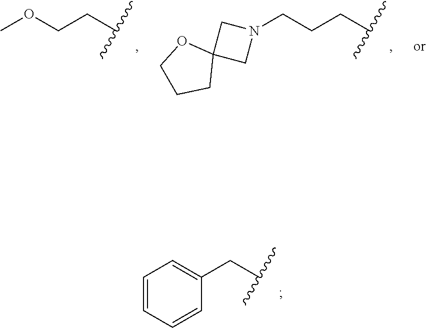

X³ is H, methyl, or ethyl; R² is isopropyl; R¹ is

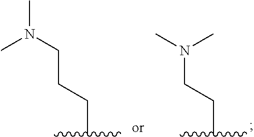

and n is 1, 2, or 3.

In some embodiments, W is CH; Y is N; Z is O; Ring A is

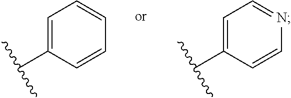 [SMILES: *c1ccccc1 ; "or" ; *c1ccncc1]

R³ is H, methyl, ethyl, isopropyl, tert-butyl,

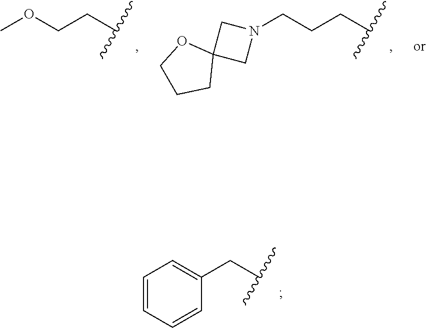

X³ is H, methyl, or ethyl; R² is isopropyl; R¹ is

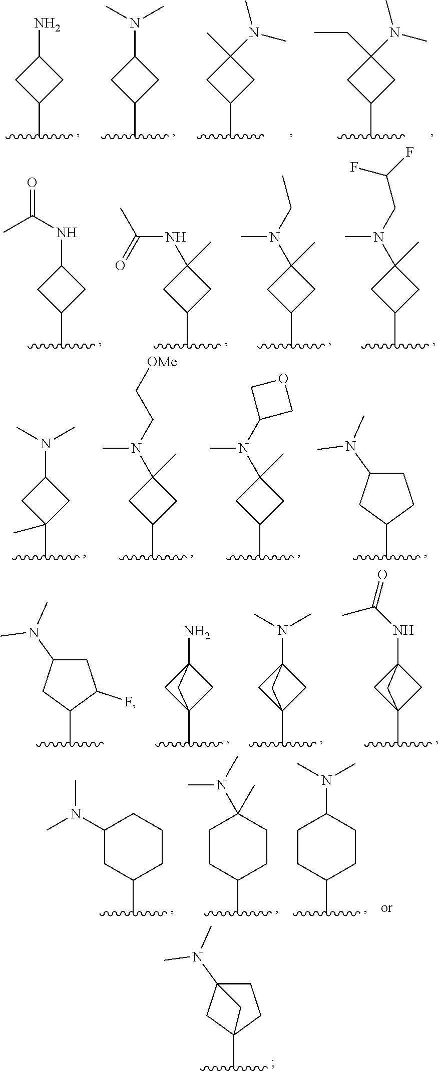

and n is 1, 2, or 3.

In some embodiments, W is CH; Y is N; Z is O Ring A is

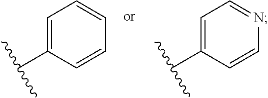 [SMILES: *c1ccccc1 ; "or" ; *c1ccncc1]

R³ is H, methyl, ethyl, isopropyl, tert-butyl,

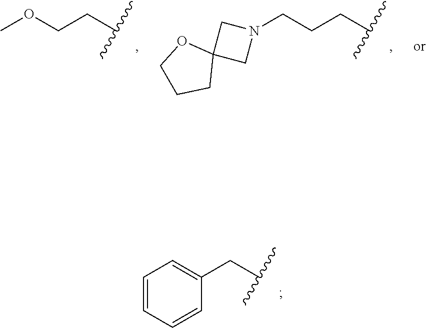

X³ is H, methyl, or ethyl; R² is isopropyl, R¹ is

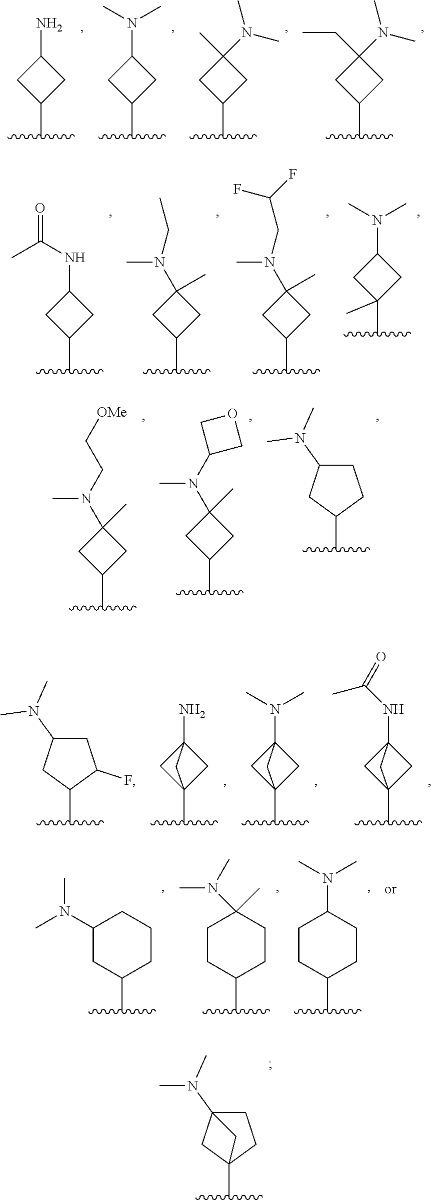

and n is 1, 2, or 3.

In some embodiments, W is CH; Y is N; Z is O; Ring A is

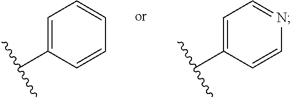 [SMILES: *c1ccccc1 ; "or" ; *c1ccncc1]

R³ is H, methyl, ethyl, isopropyl, tert-butyl,

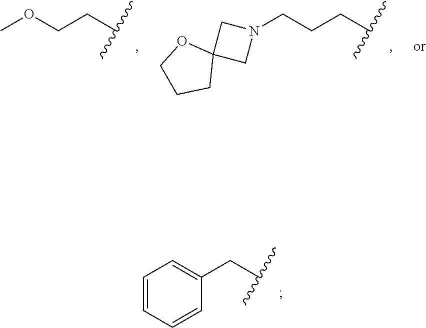

X³ is H, methyl, or ethyl; R² is isopropyl; R¹ is

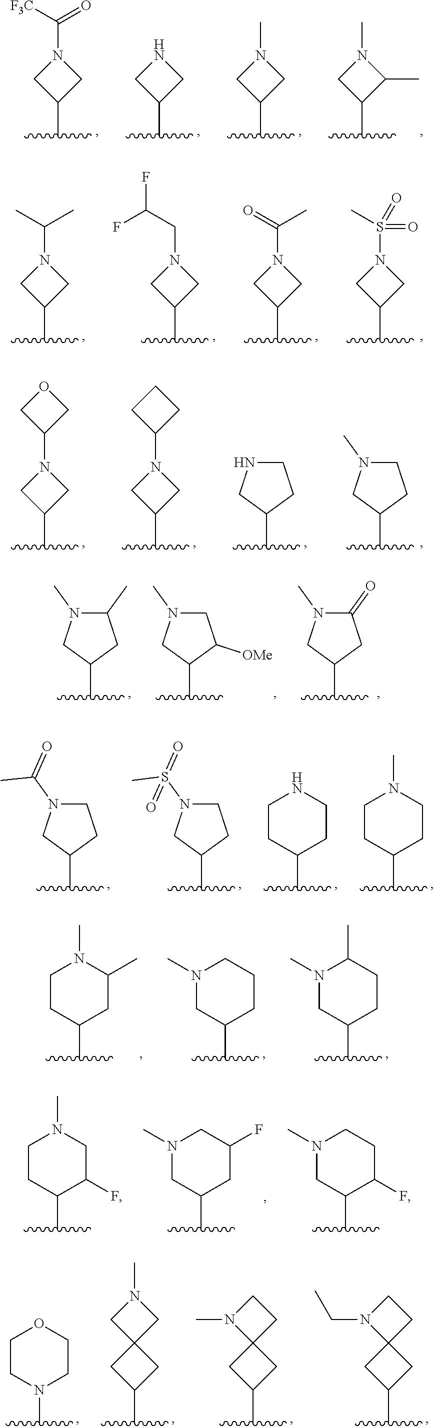

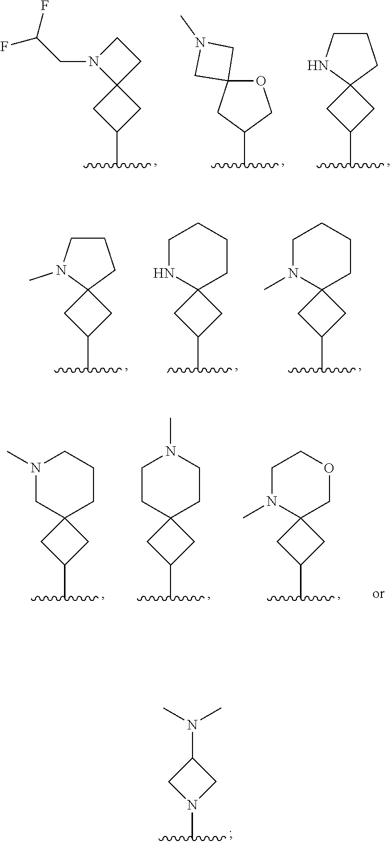

n is 1, 2, or 3.

In some embodiments, W is CH; Y is N; Z is O; Ring A is

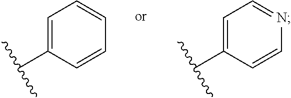 [SMILES: *c1ccccc1 ; "or" ; *c1ccncc1]

R³ is H, methyl, ethyl, isopropyl, tert-butyl,

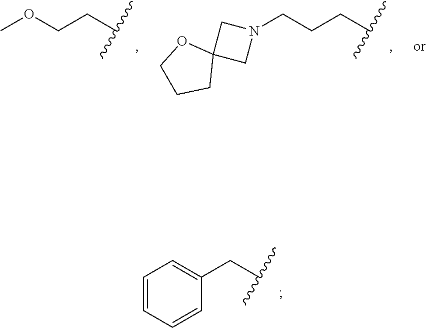

X³ is H, methyl, or ethyl; R² is isopropyl; R¹ is

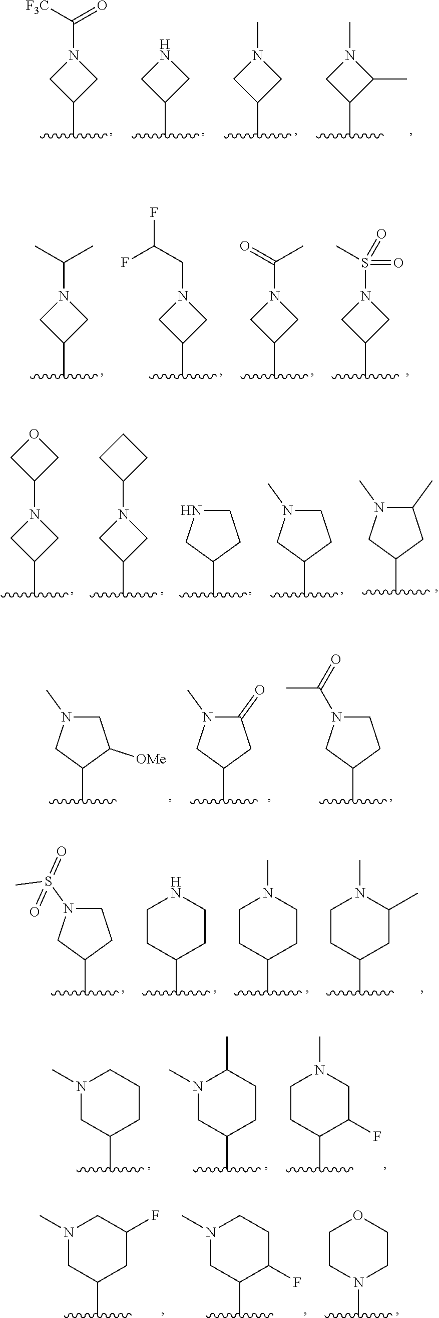

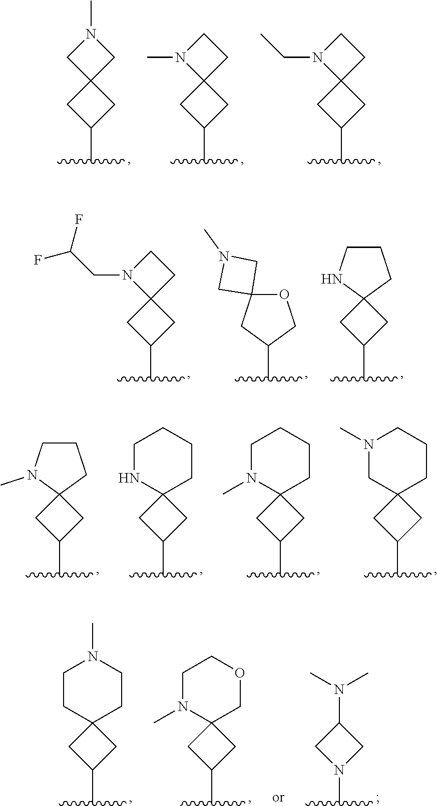

and n is 1, 2, or 3.

In some embodiments, W is CH; Y is N; Z is O; Ring A is

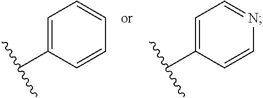 [SMILES: *c1ccccc1 ; *c1ccncc1]

R³ is H, methyl, ethyl, isopropyl, tert-butyl,

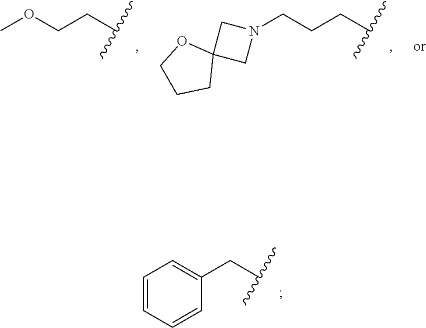

X³ is H, methyl, or ethyl; R² is isopropyl; R¹ is

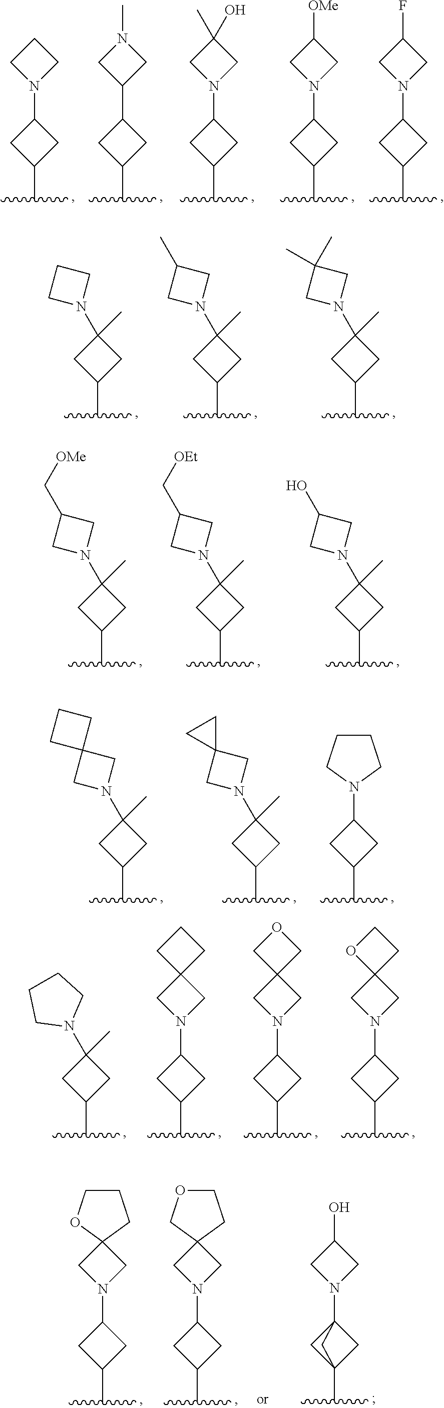

and n is 1, 2, or 3.

In some embodiments, W is CH, Y is N, Z is O,

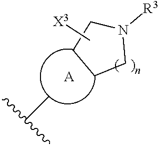

is

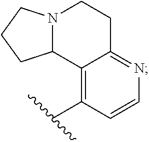 [SMILES: *c1ccnc2c1C1CCCN1CC2]

and n is 1, 2, or 3.

In some embodiments, W is CH; Y is N; Z is O;

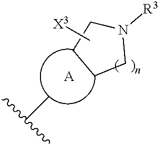

is

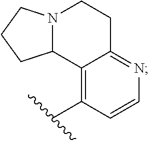 [SMILES: *c1ccnc2c1C1CCCN1CC2]

R² is isopropyl; R¹ is

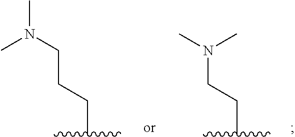

and n is 1, 2, or 3.

In some embodiments, W is CH; Y is N; Z is O;

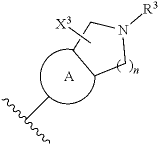

is

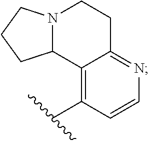 [SMILES: *c1ccnc2c1C1CCCN1CC2]

R² is isopropyl; R¹ is

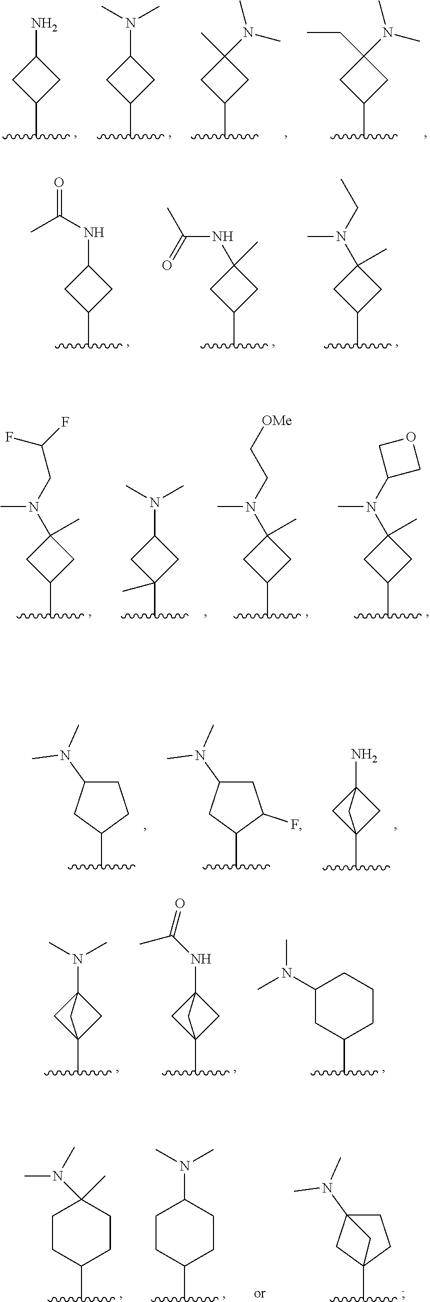

and n is 1, 2, or 3.

In some embodiments, W is CH; Y is N; Z is O;

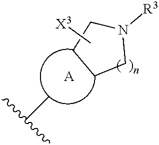

is

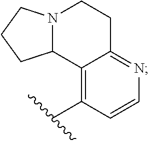 [SMILES: *c1ccnc2c1C1CCCN1CC2]

R² is isopropyl; R¹ is

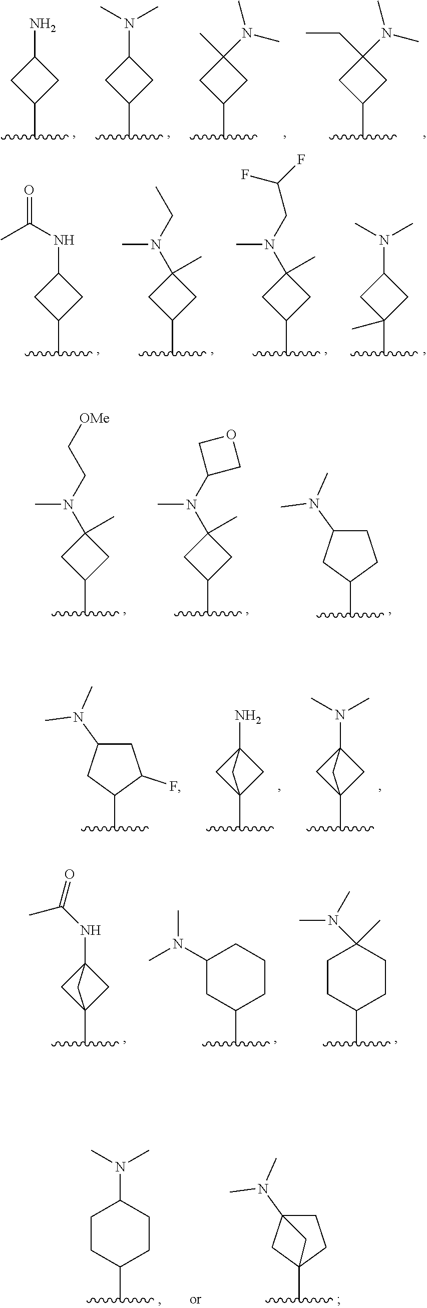

and n is 1, 2, or 3.

In some embodiments, W is CH; Y is N; Z is O;

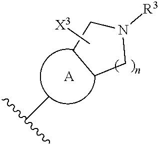

is

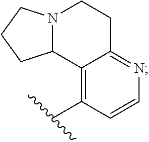 [SMILES: *c1ccnc2c1C1CCCN1CC2]

R² is isopropyl; R¹ is

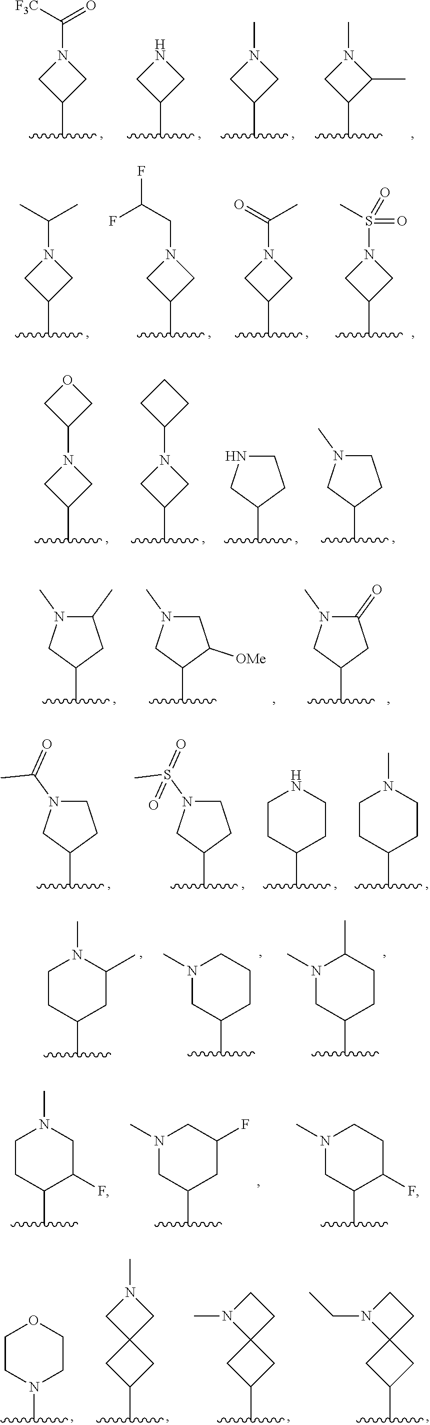

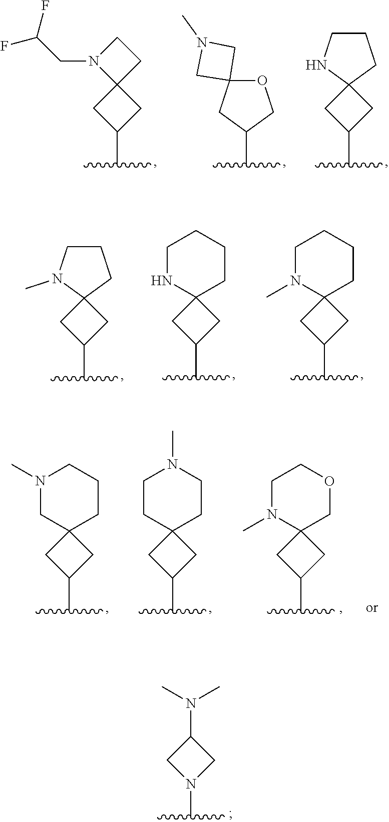

and n is 1, 2, or 3.

In some embodiments, W is CH; Y is N; Z is O;

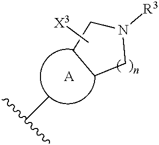

is

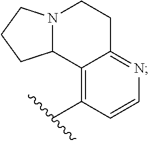 [SMILES: *c1ccnc2c1C1CCCN1CC2]

R² is isopropyl; R¹ is

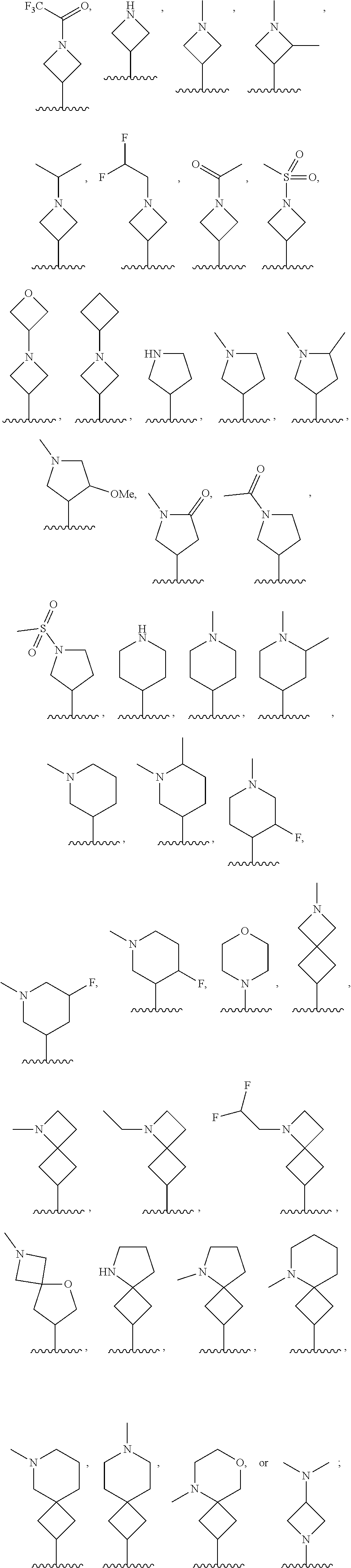

and n is 1, 2, or 3.

In some embodiments, W is CH; Y is N; Z is O;

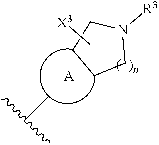

is

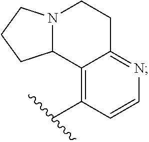 [SMILES: *c1ccnc2c1C1CCCN1CC2]

R² is isopropyl; R¹ is

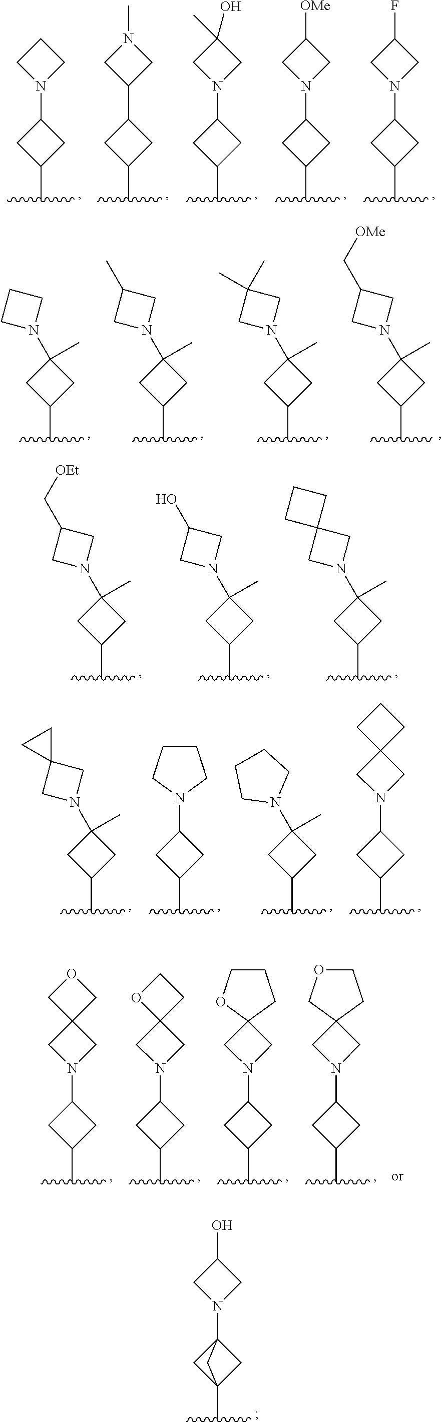

and n is 1, 2, or 3.

In some embodiments, W is CH; Y is N; Z is O; Ring A is

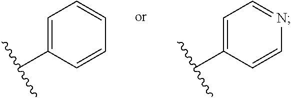 [SMILES: *c1ccccc1 ; "or" ; *c1ccncc1]

### R³ is

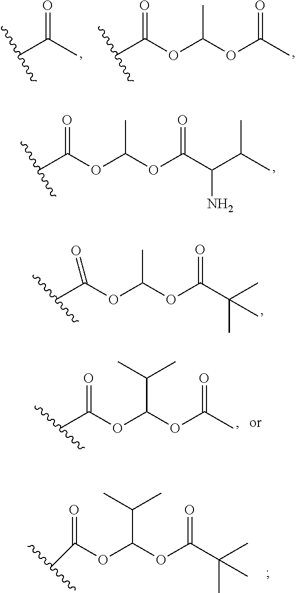

and X³ is H, methyl, or ethyl.

In some embodiments, W is CH; Y is N; Z is O; Ring A is

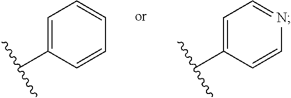 [SMILES: *c1ccccc1 ; "or" ; *c1ccncc1]

### R³ is

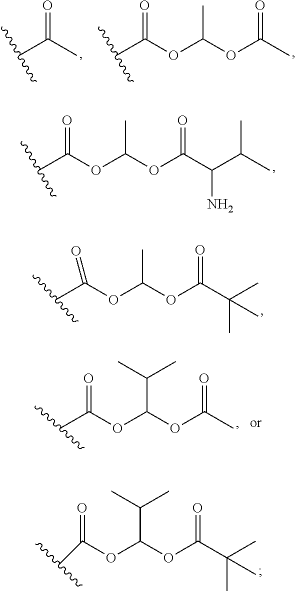

X³ is H, methyl, or ethyl; R² is isopropyl; and R¹ is

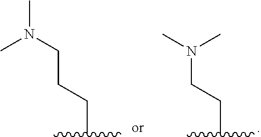

In some embodiments, W is CH; Y is N; Z is O; Ring A is

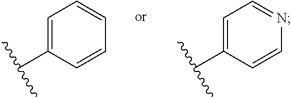 [SMILES: *c1ccccc1 ; "or" ; *c1ccncc1]

### R³ is

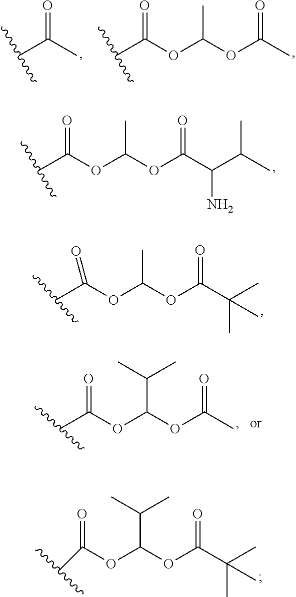

X³ is H, methyl, or ethyl; R² is isopropyl; and R¹ is

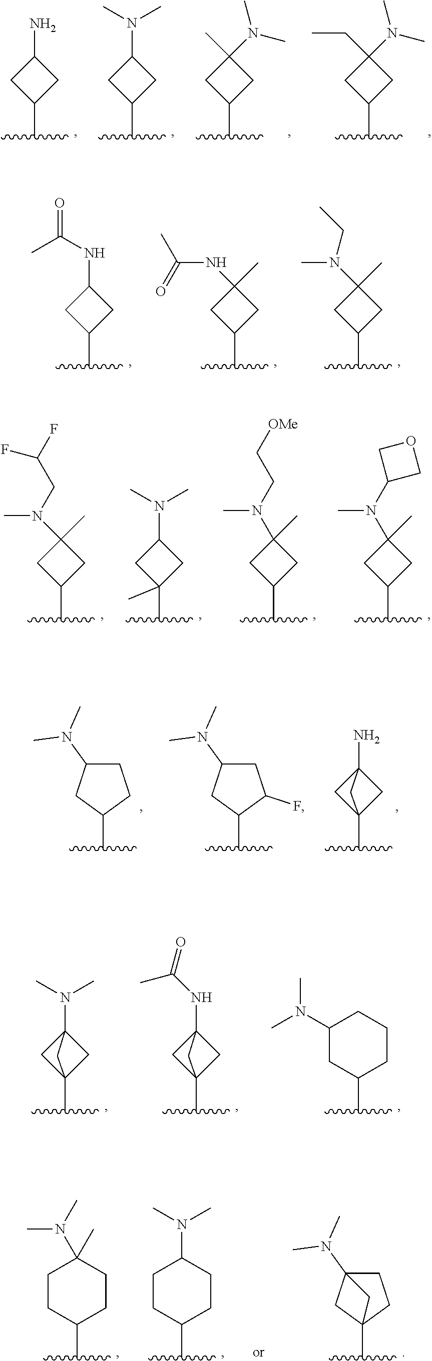

In some embodiments, W is CH; Y is N; Z is O; Ring A is

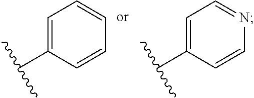 [SMILES: *c1ccccc1 ; "or" ; *c1ccncc1]

### R³ is

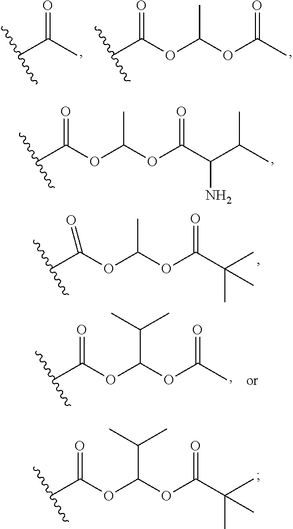

X is H, methyl, or ethyl; R² is isopropyl; and R¹ is

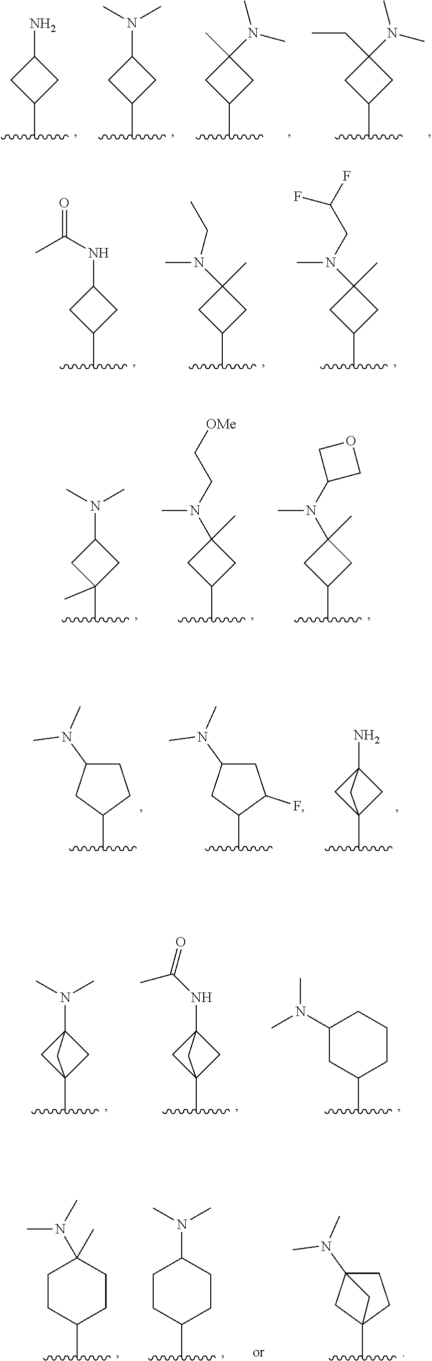

In some embodiments, W is CH; Y is N; Z is O; Ring A is

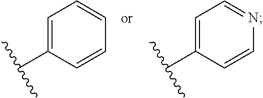 [SMILES: *c1ccccc1 ; *c1ccncc1]

### R³

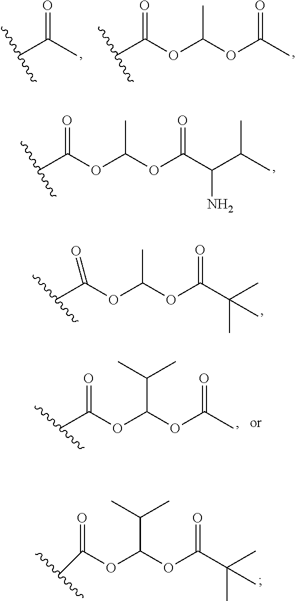

X³ is H, methyl, or ethyl; R² is isopropyl; and R¹ is

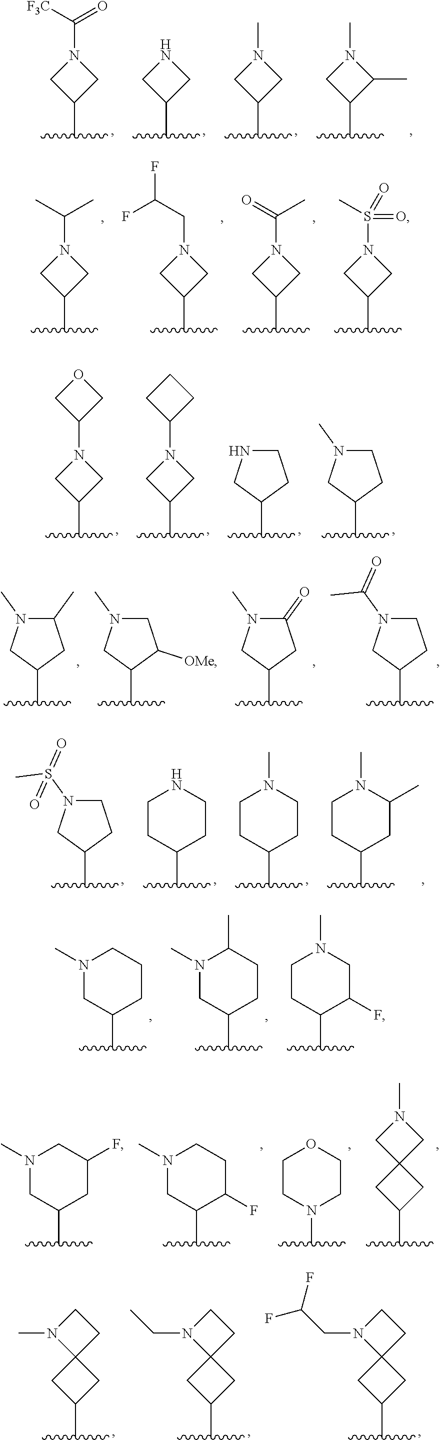

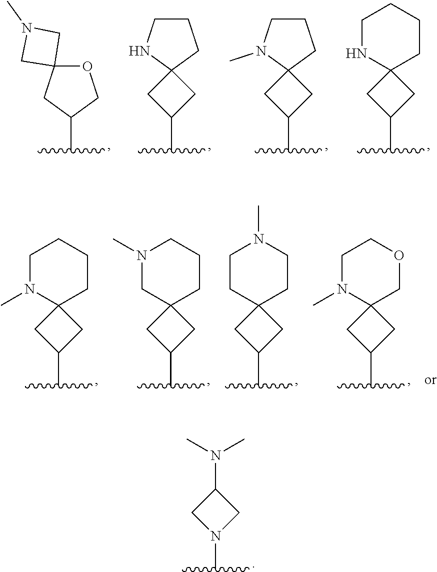

In some embodiments, W is CH; Y is N; Z is O; Ring A is

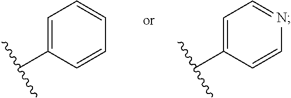 [SMILES: *c1ccccc1 ; *c1ccncc1]

### R³

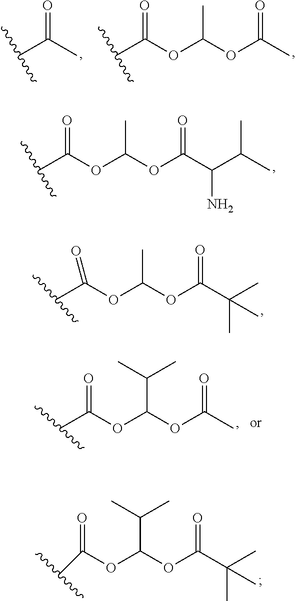

### R³

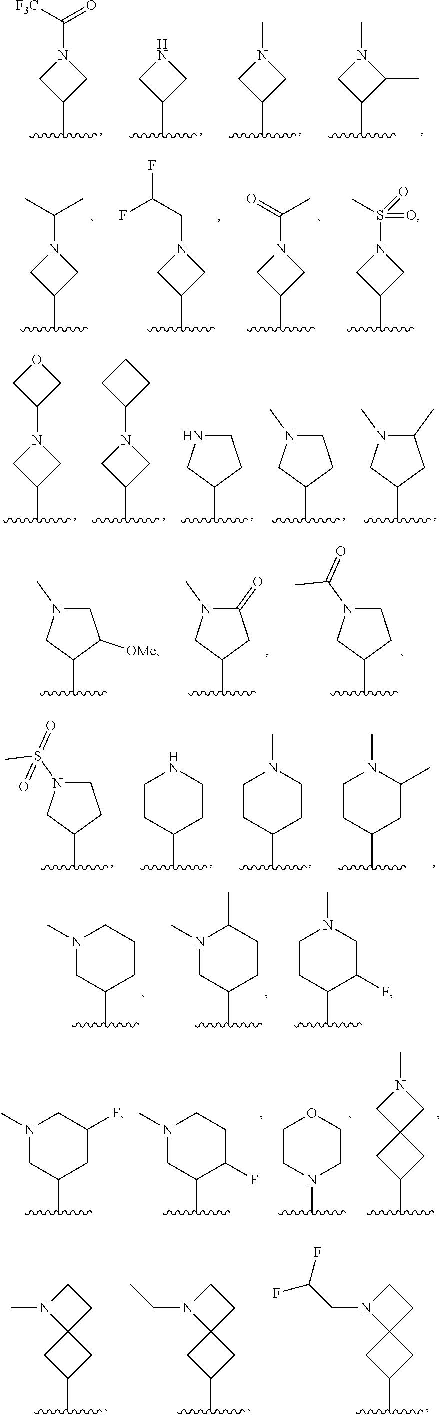

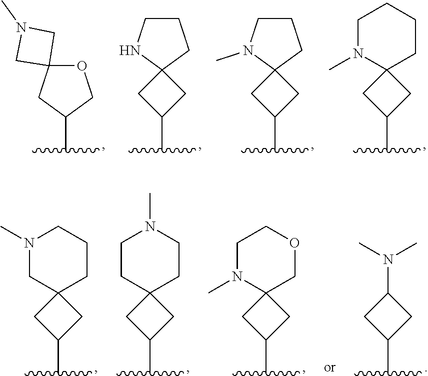

In some embodiments, W is CH; Y is N; Z is O; Ring A is

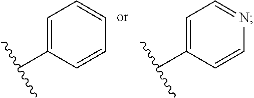 [SMILES: *c1ccccc1 ; "or" ; *c1ccncc1]

### R³ is

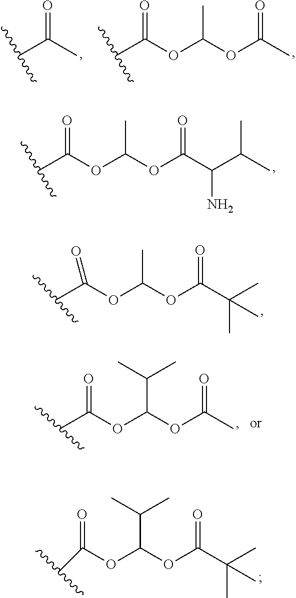

X³ is H, methyl, or ethyl; R² is isopropyl; and R¹ is

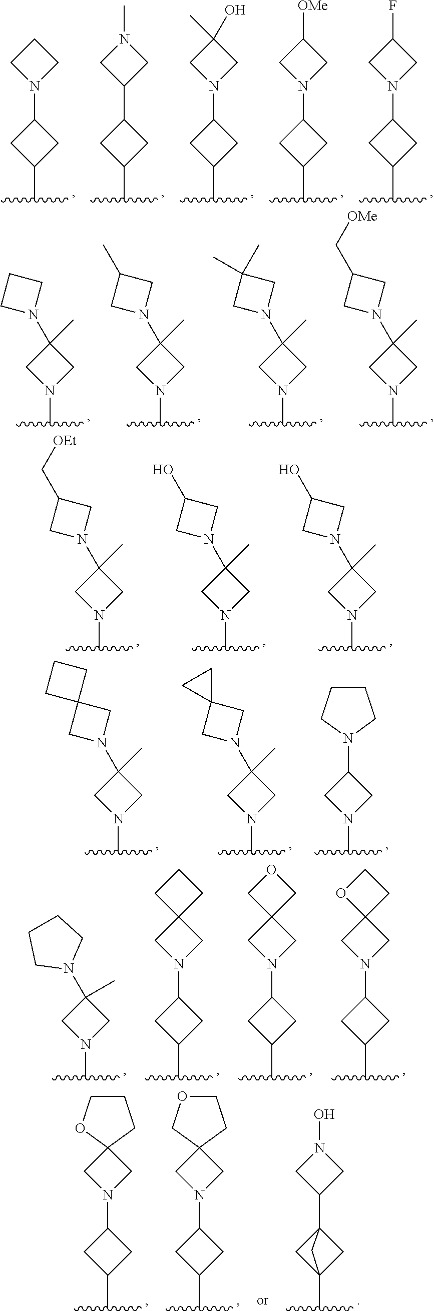

In some embodiments, W is CH; Y is N; Z is O; Ring A is

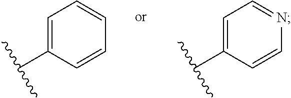 [SMILES: *c1ccccc1 ; "or" ; *c1ccncc1]

### R³ is

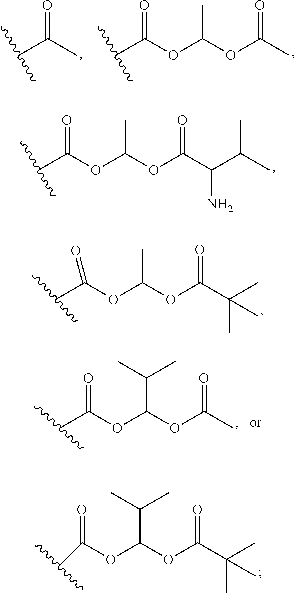

X³ is H, methyl, or ethyl; and n is 1, 2, or 3.

In some embodiments, W is CH; Y is N; Z is O; Ring A is

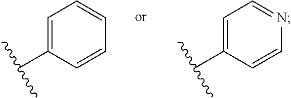 [SMILES: *c1ccccc1 ; "or" ; *c1ccncc1]

### R³ is

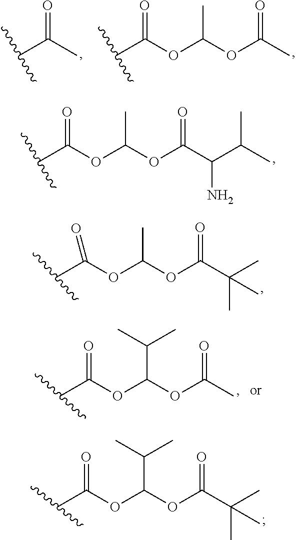

X³ is H, methyl, or ethyl; R² is isopropyl; R¹ is

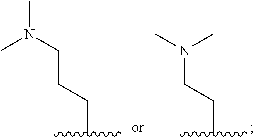

and n is 1, 2, or 3.

In some embodiments, W is CH; Y is N; Z is O; Ring A is

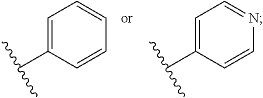 [SMILES: *c1ccccc1 ; *c1ccncc1]

### R³ is

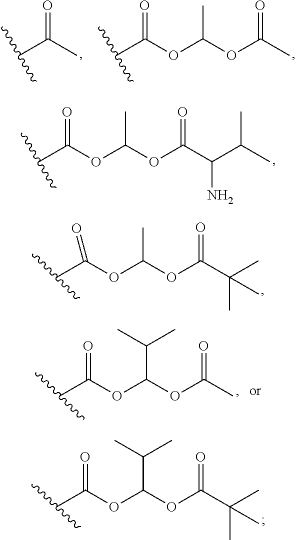

X³ is H, methyl, or ethyl; R² is isopropyl; R¹ is

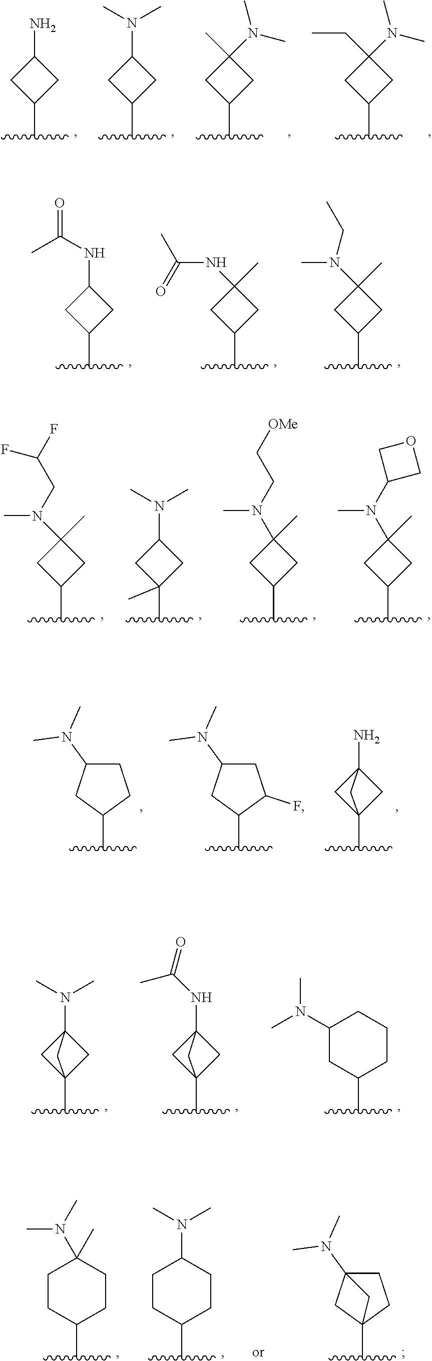

and n is 1, 2, or 3.

In some embodiments, W is CH; Y is N; Z is O; Ring A is

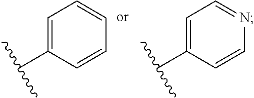 [SMILES: *c1ccccc1 ; "or" ; *c1ccncc1]

### R³ is

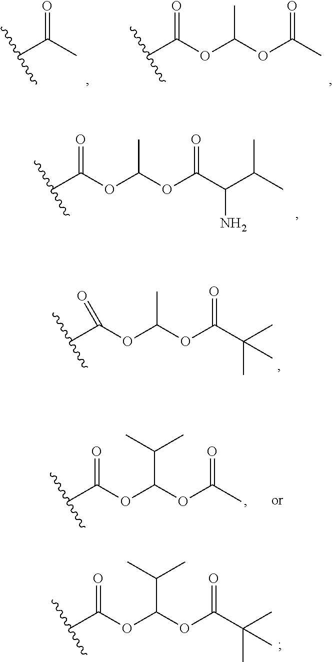

X³ is H, methyl, or ethyl; R² is isopropyl; R¹ is

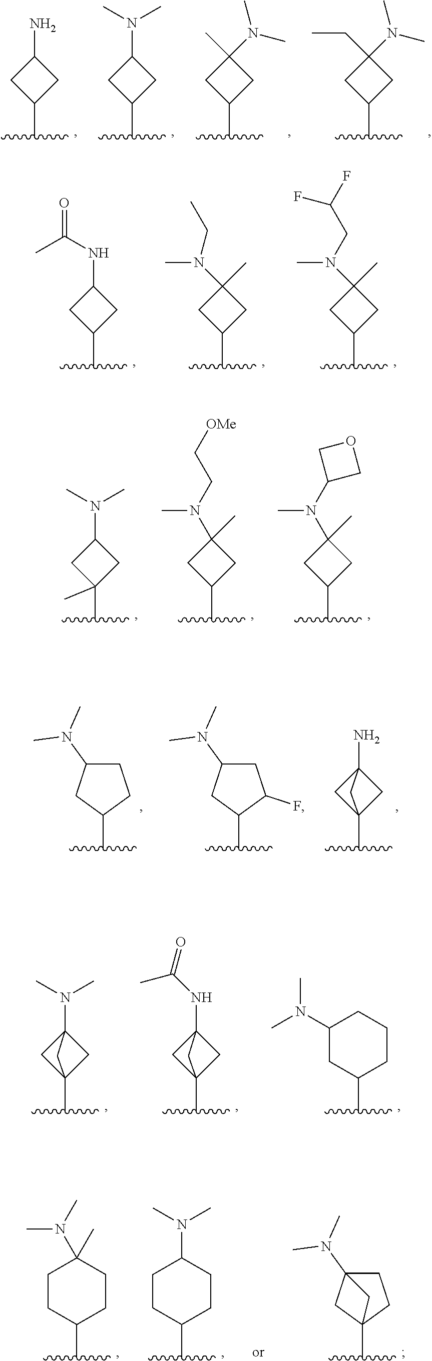

and n is 1, 2, or 3.

In some embodiments, W is CH Y is N; Z is O; Ring A is

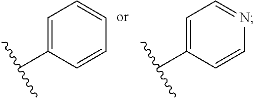 [SMILES: *c1ccccc1 ; "or" ; *c1ccncc1]

### R³ is

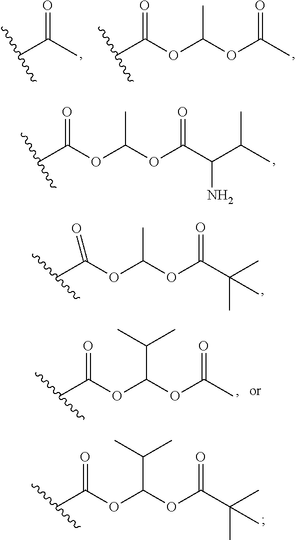

X³ is H, methyl, or ethyl; R² is isopropyl; R¹ is

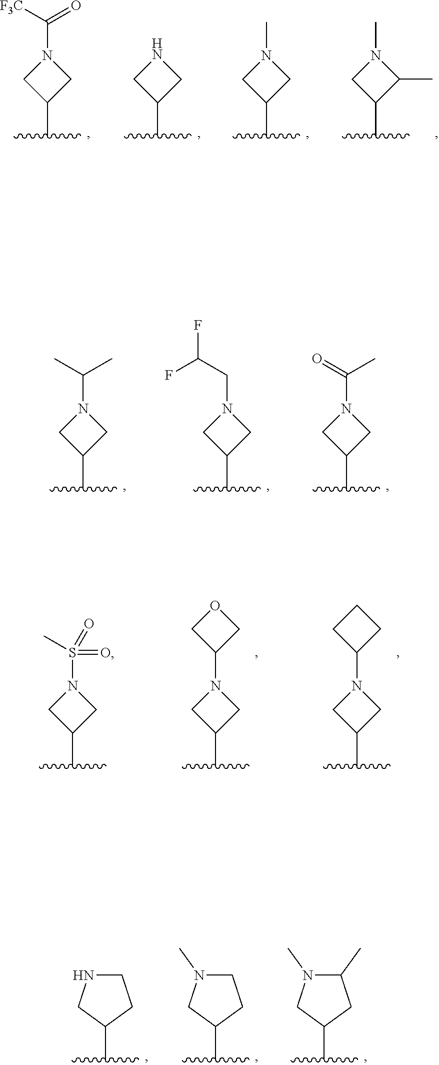

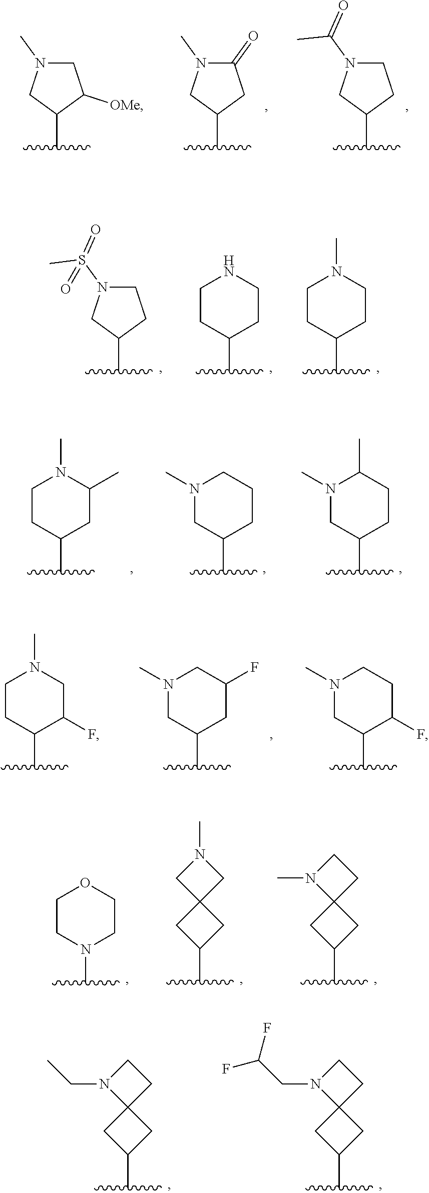

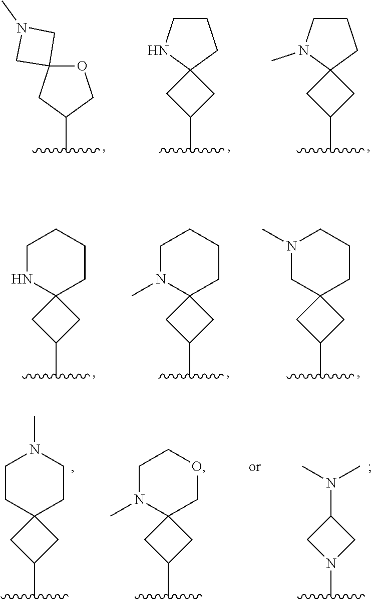

and n is 1, 2, or 3.

In some embodiments, W is CH; Y is N; Z is O; Ring A is

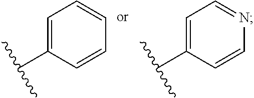 [SMILES: *c1ccccc1 ; "or" ; *c1ccncc1]

### R³ is

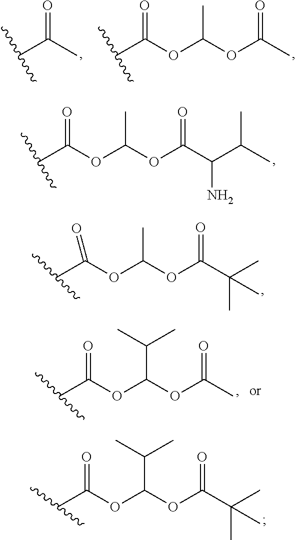

X³ is H, methyl, or ethyl; R² is isopropyl; R¹ is

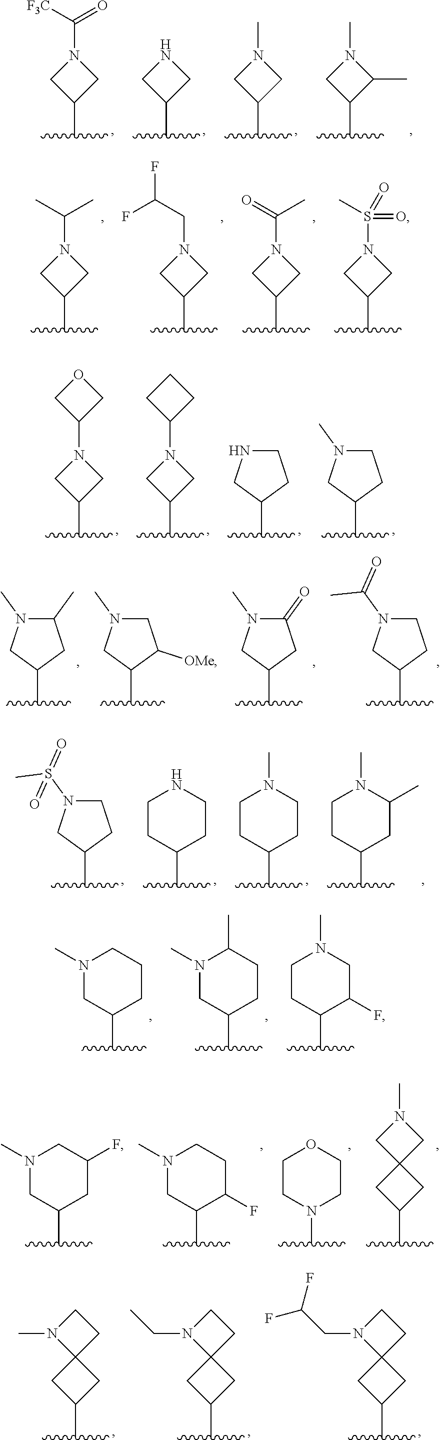

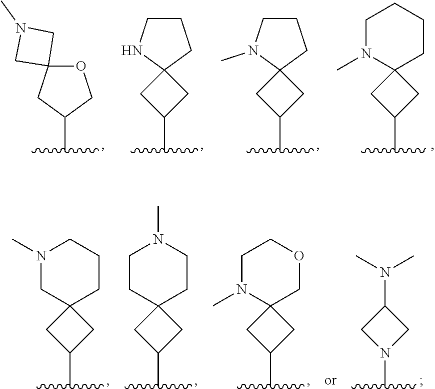

and n is 1, 2, or 3.

In some embodiments, W is CH; Y is N; Z is O, Ring A is

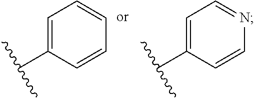 [SMILES: *c1ccccc1 ; "or" ; *c1ccncc1]

### R³ is

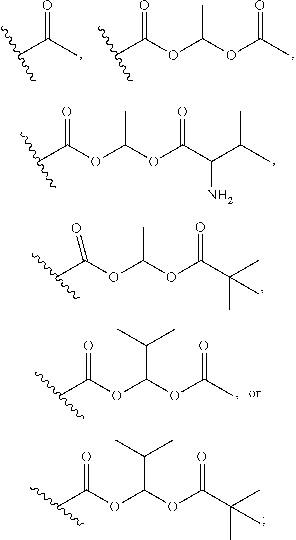

X³ is H, methyl, or ethyl; R² is isopropyl; R¹ is

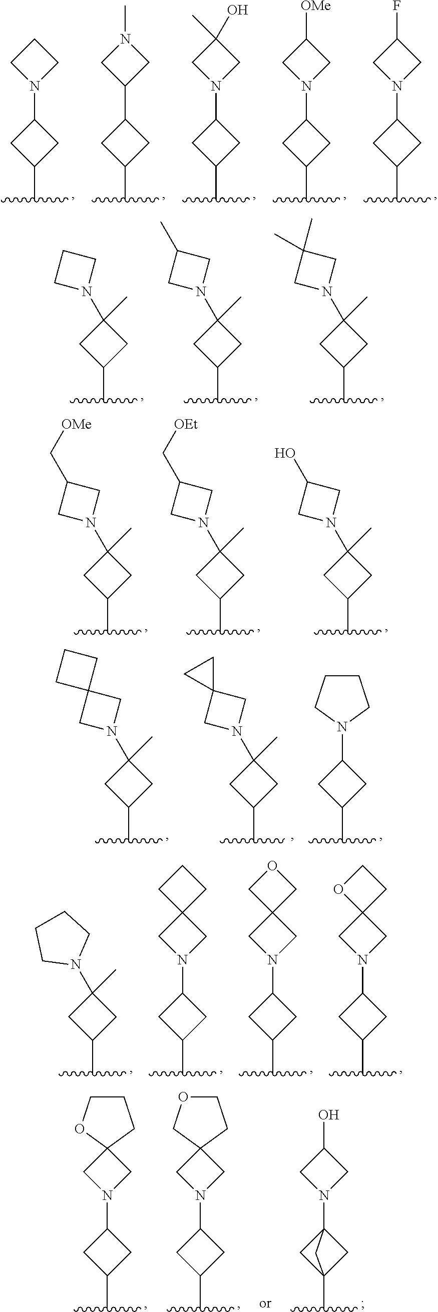

and n is 1, 2, or 3.

In some embodiments, W is N, Y is N, Z is O, and Ring A is

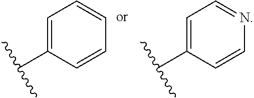 [SMILES: *c1ccccc1 ; "or" ; *c1ccncc1]

In some embodiments, W is N; Y is N; Z is O; Ring A is

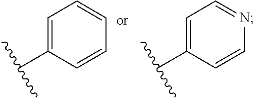 [SMILES: *c1ccccc1 ; *c1ccncc1]

R² is isopropyl; and R¹ is

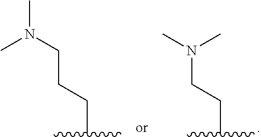

In some embodiments, W is N; Y is N; Z is O; Ring A is

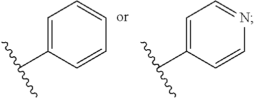 [SMILES: *c1ccccc1 ; "or" ; *c1ccncc1]

R² is isopropyl; and R¹ is

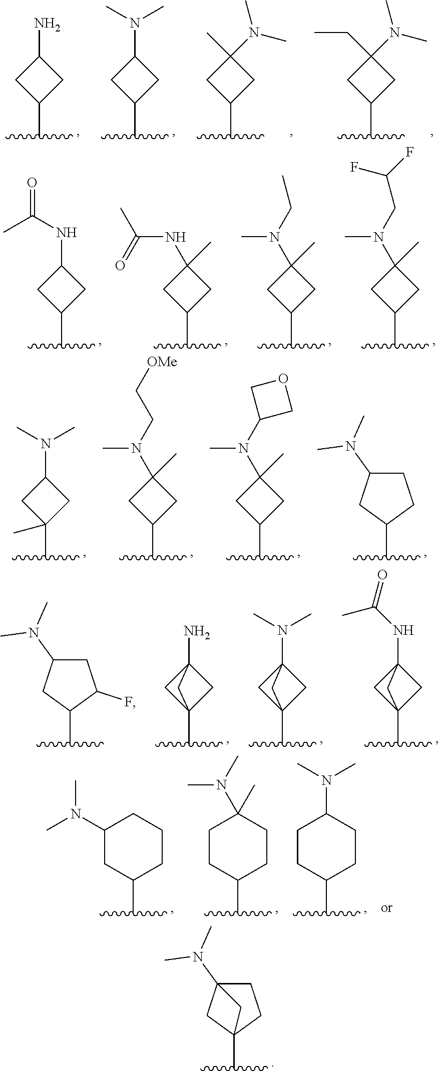

In some embodiments, W is N; Y is N; Z is O; Ring A is

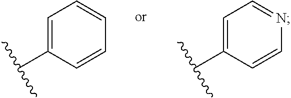 [SMILES: *c1ccccc1 ; "or" ; *c1ccncc1]

R² is isopropyl; and R¹ is

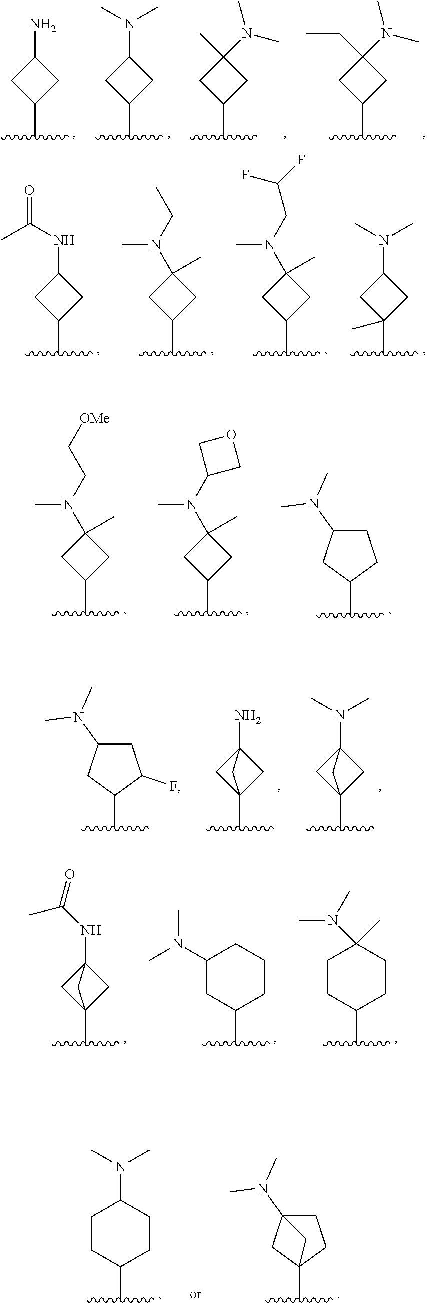

In some embodiments, W is N; Y is N; Z is O; Ring A is

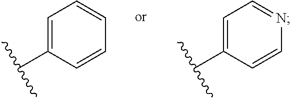 [SMILES: *c1ccccc1 ; "or" ; *c1ccncc1]

R² is-52 isopropyl; and R¹ is

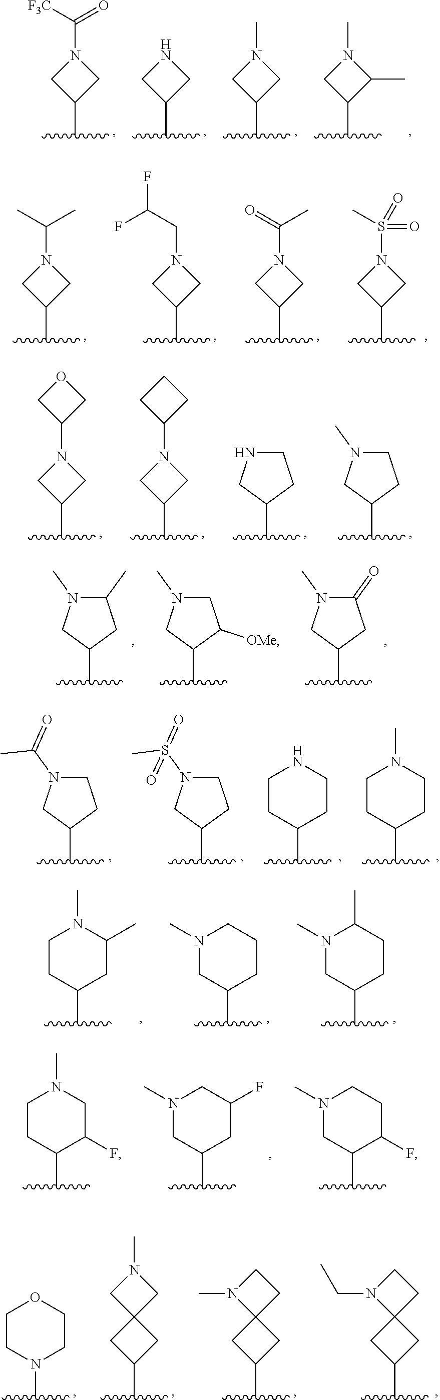

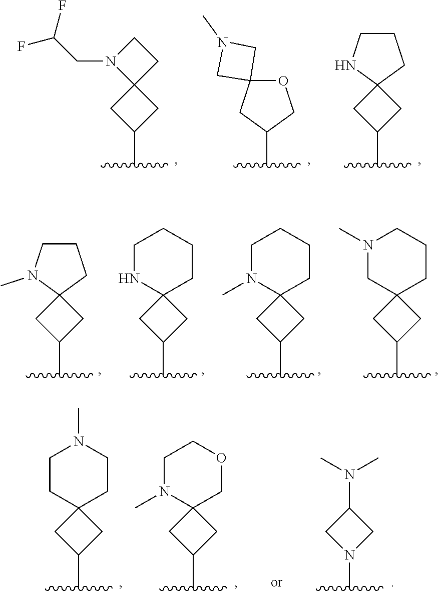

In some embodiments, W is N; Y is N; Z is O; Ring A is

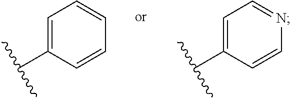 [SMILES: *c1ccccc1 ; "or" ; *c1ccncc1]

R² is isopropyl; and R¹ is

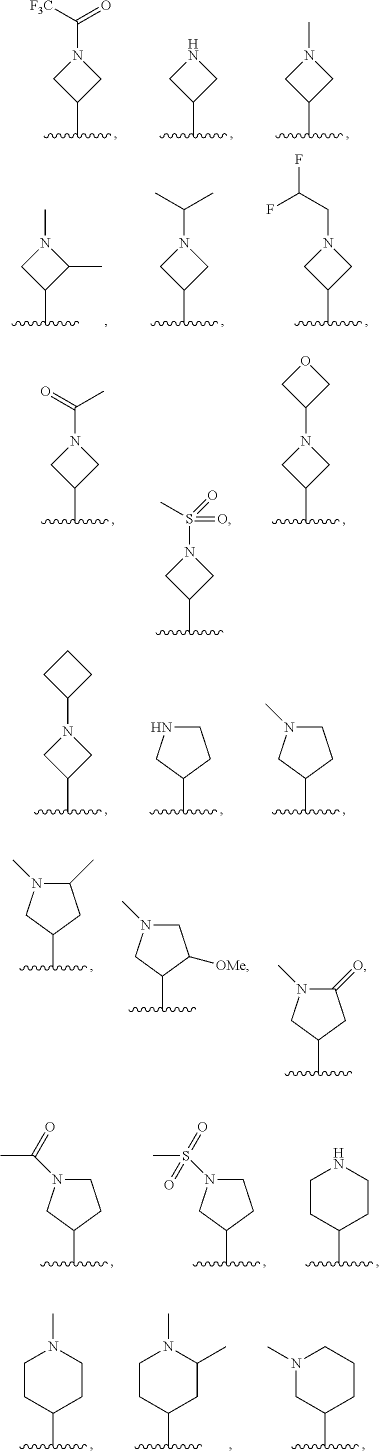

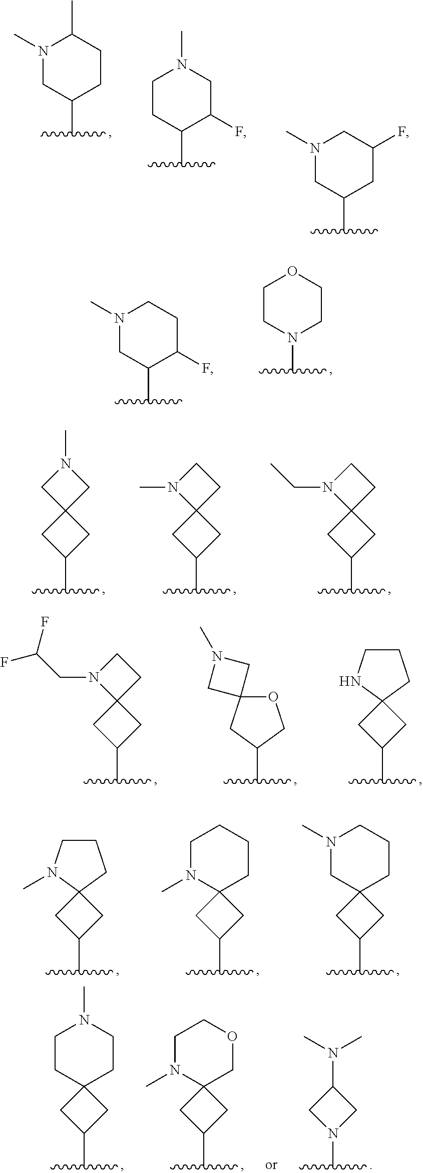

In some embodiments, W is N; Y is N, Z is O; Ring A is

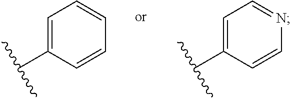 [SMILES: *c1ccccc1 ; "or" ; *c1ccncc1]

R² is isopropyl; and R¹ is

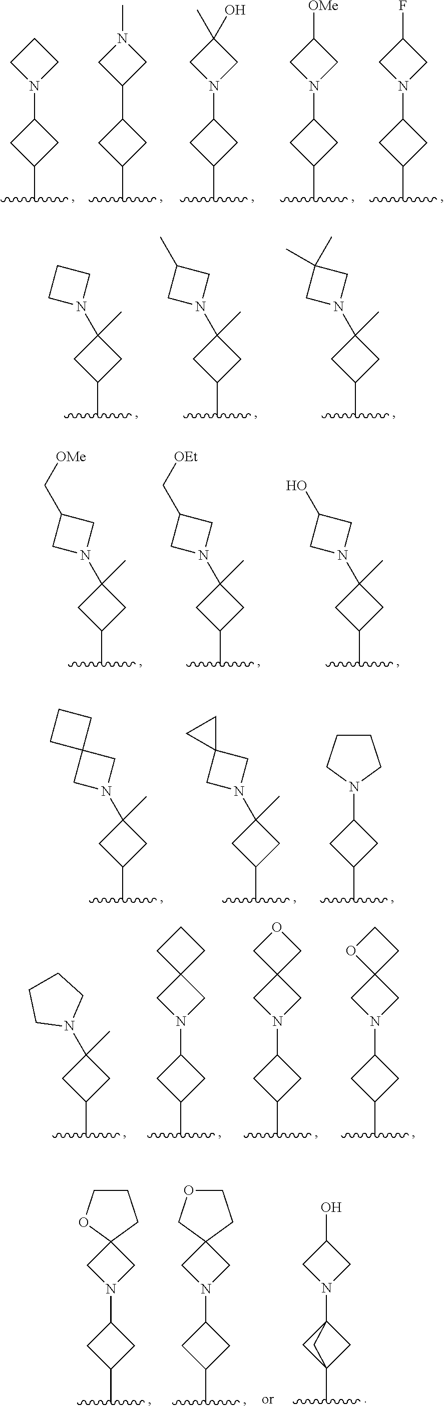

In some embodiments, W is N; Y is N, Z is O; Ring A is

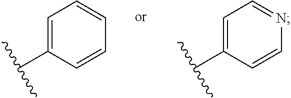 [SMILES: *c1ccccc1 ; "or" ; *c1ccncc1]

and X³ is H, methyl, or ethyl.

In some embodiments, W is N, Y is N; Z is O, Ring A is

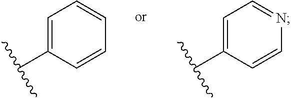 [SMILES: *c1ccccc1 ; "or" ; *c1ccncc1]

X³ is H, methyl, or ethyl; R² is isopropyl; and R¹ is

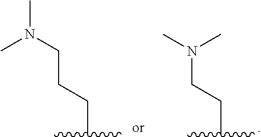

In some embodiments, W is N; Y is N; Z is O; Ring A is

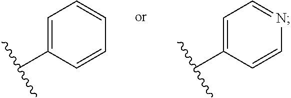 [SMILES: *c1ccccc1 ; "or" ; *c1ccncc1]

X³ is H, methyl, or ethyl; R² is isopropyl; and R¹ is

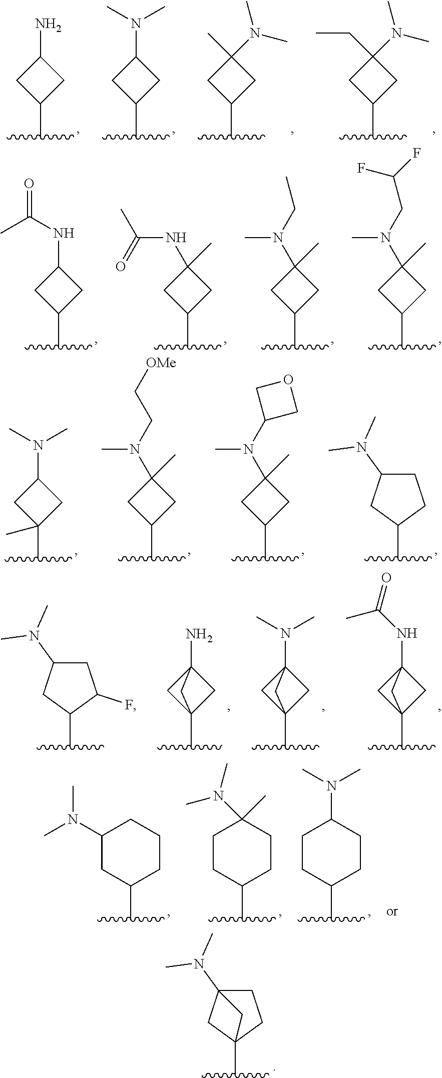

In some embodiments, W is N; Y is N; Z is O; Ring A is

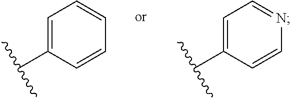 [SMILES: *c1ccccc1 ; "or" ; *c1ccncc1]

X³ is H, methyl, or ethyl; R² is isopropyl; and R¹ is

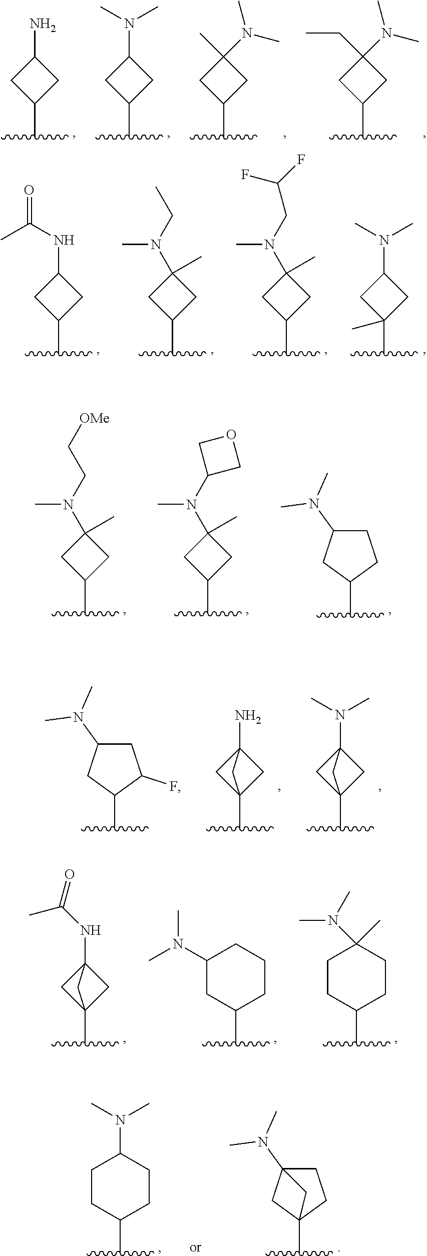

In some embodiments, W is N; Y is N; Z is O; Ring A is

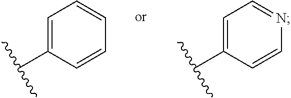 [SMILES: *c1ccccc1 ; "or" ; *c1ccncc1]

X³ is H, methyl, or ethyl; R is isopropyl; and R¹ is

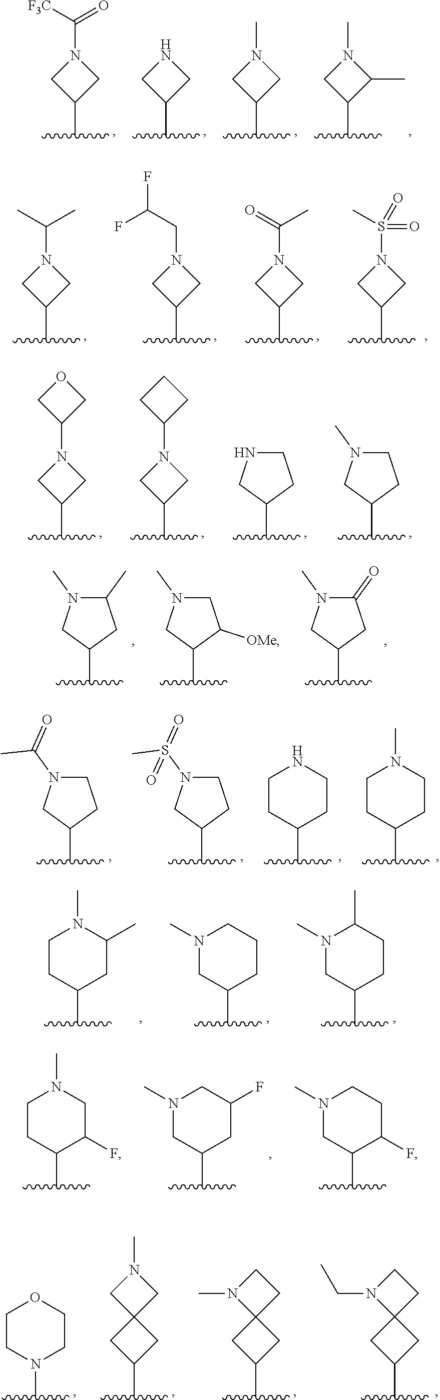

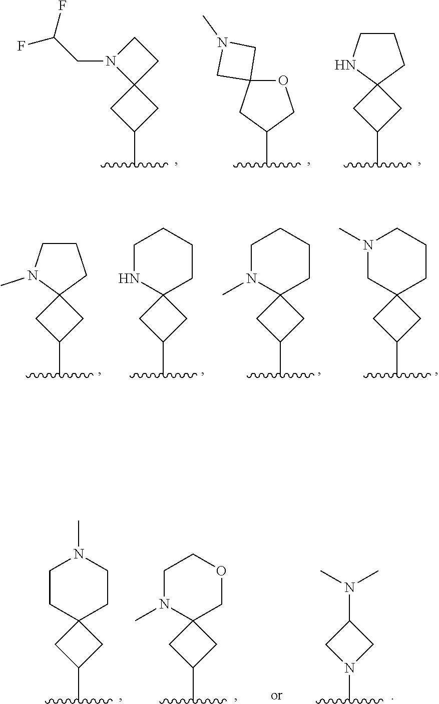

In some embodiments, W is N; Y is N; Z is O; Ring A is

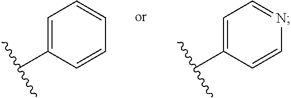 [SMILES: *c1ccccc1 ; "or" ; *c1ccncc1]

X³ is H, methyl, or ethyl; R² is isopropyl; and R¹ is

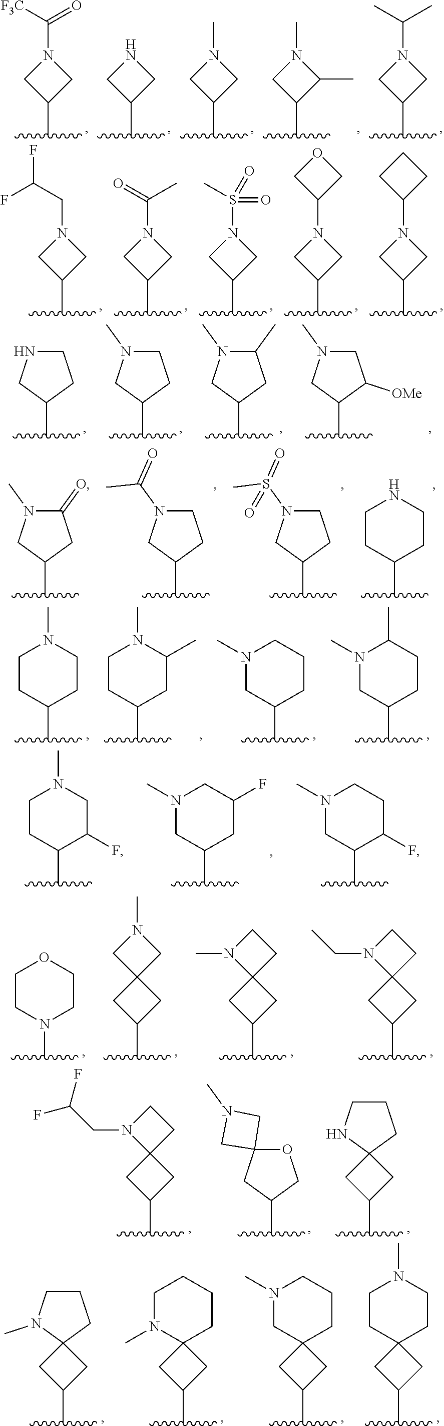

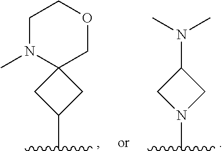

In some embodiments, W is N; Y is N; Z is O; Ring A is

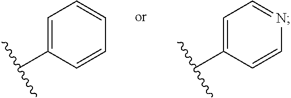 [SMILES: *c1ccccc1 ; "or" ; *c1ccncc1]

X³ is H, methyl, or ethyl; R² is isopropyl; and R¹ is

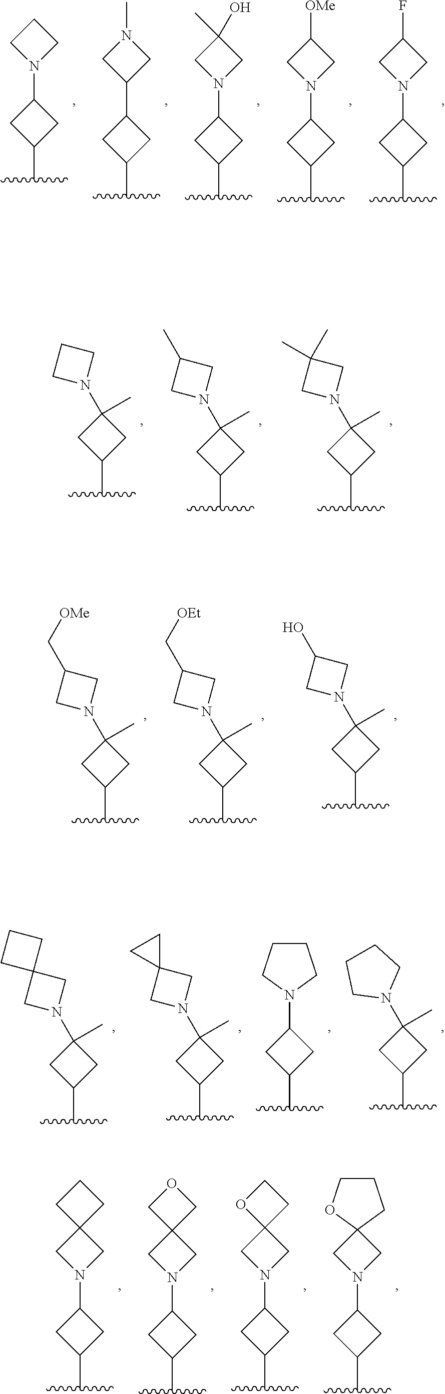

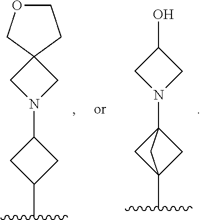 [SMILES: *C1CC(N2CC3(CCOC3)C2)C1 ; *C12CC(N3CC(O)C3)(C1)C2]

In some embodiments, W is N; Y is N, Z is O; Ring A is

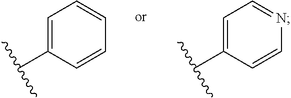 [SMILES: *c1ccccc1 ; "or" ; *c1ccncc1]

R³ is H, methyl, ethyl, isopropyl, tert-butyl,

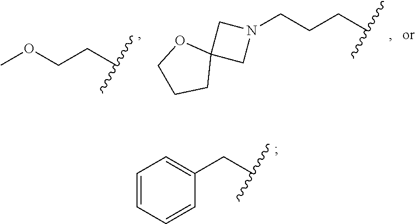

and X³ is H, methyl, or ethyl.

In some embodiments, W is N; Y is N; Z is O; Ring A is

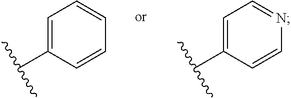 [SMILES: *c1ccccc1 ; "or" ; *c1ccncc1]

R³ is H, methyl, ethyl, isopropyl, tert-butyl,

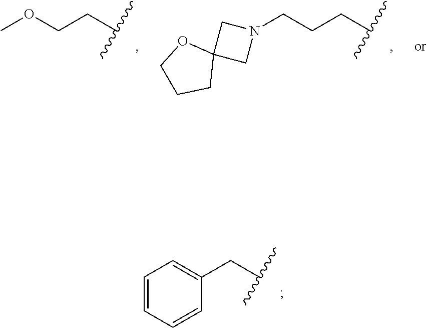

X³ is H, methyl, or ethyl; R² is isopropyl; and R¹ is

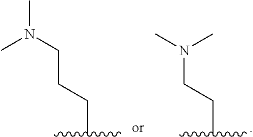

In some embodiments, W is N; Y is N; Z is O; Ring A is

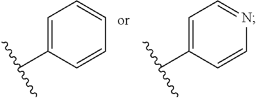 [SMILES: *c1ccccc1 ; "or" ; *c1ccncc1]

R³ is H, methyl, ethyl, isopropyl, tert-butyl,

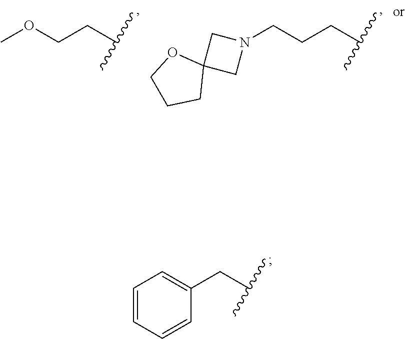

X³ is H, methyl, or ethyl; R² is isopropyl, and R¹ is

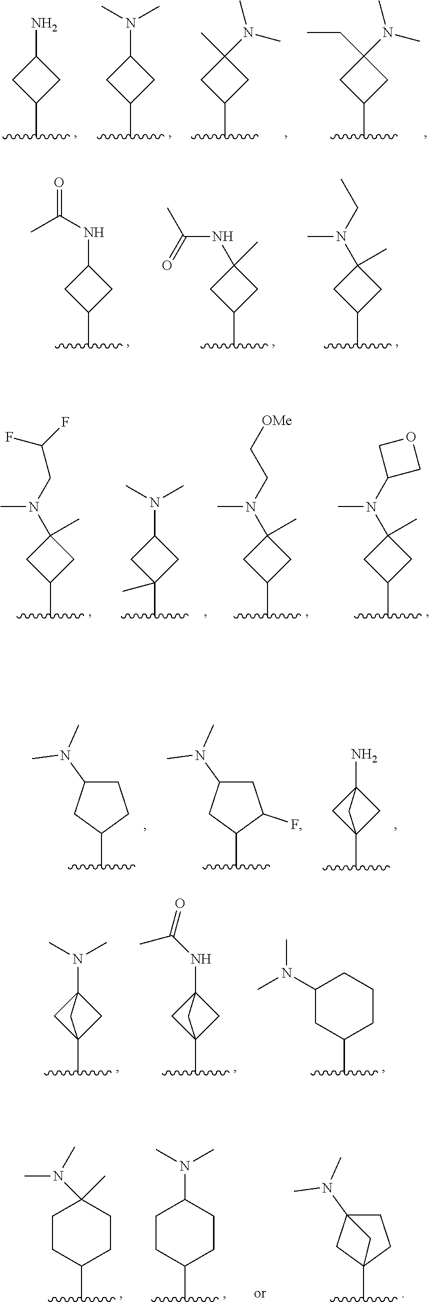

In some embodiments, W is N; Y is N; Z is O; Ring A is

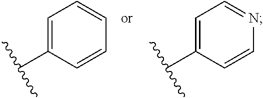 [SMILES: *c1ccccc1 ; *c1ccncc1]

R³ is H, methyl, ethyl, isopropyl, tert-butyl,

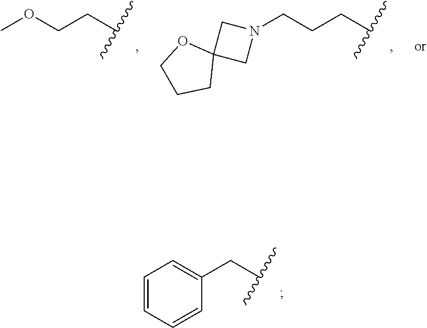

X³ is H, methyl, or ethyl; R² is isopropyl; and R¹ is

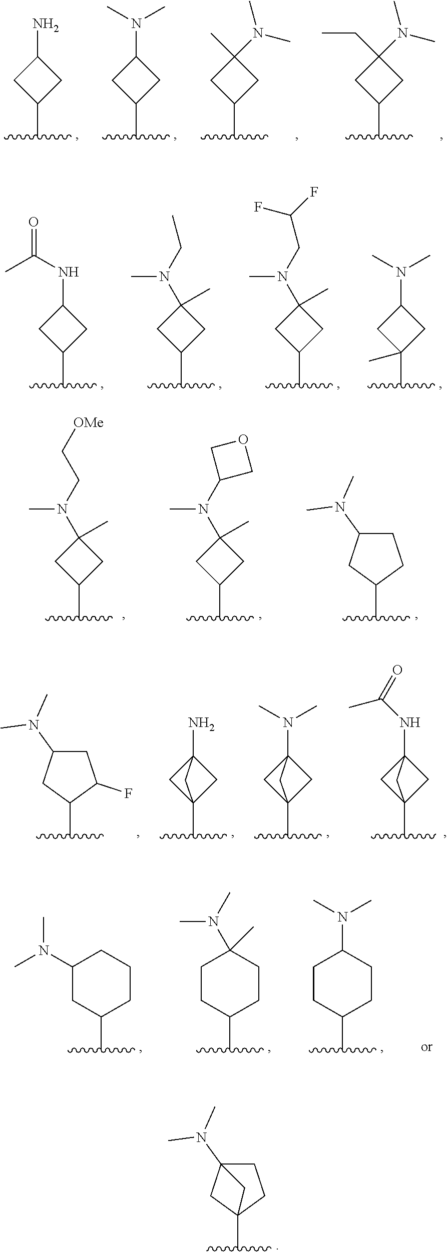

In some embodiments, W is N; Y is N; Z is O; Ring A is

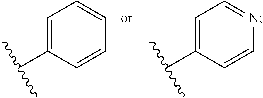 [SMILES: *c1ccccc1 ; *c1ccncc1]

R³ is H, methyl, ethyl, isopropyl, tert-butyl,

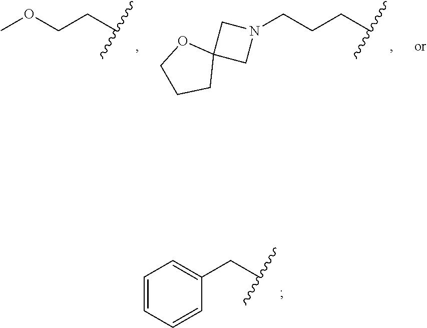

X³ is H, methyl, or ethyl; R² is isopropyl; and R¹ is

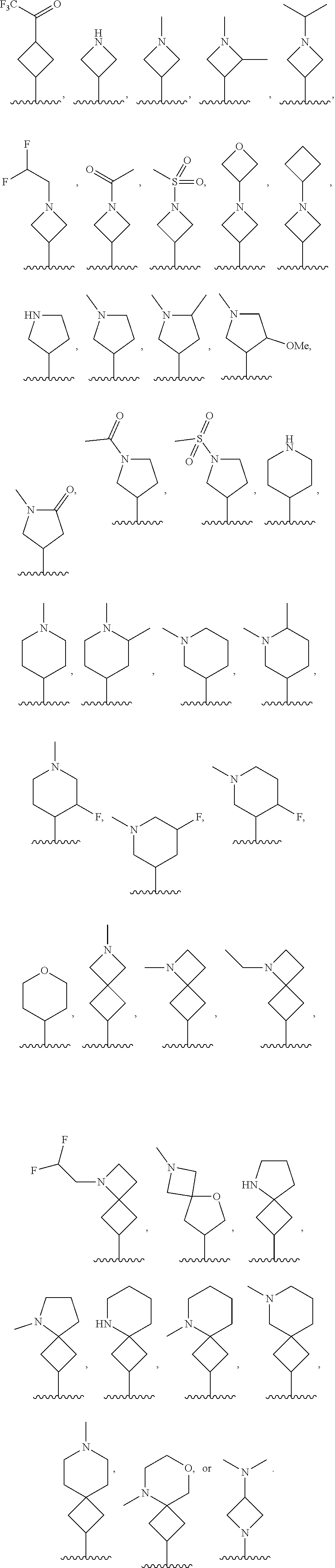

In some embodiments, W is N; Y is N; Z is O; Ring A is

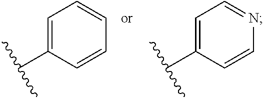 [SMILES: *c1ccccc1 ; *c1ccncc1]

R³ is H, methyl, ethyl, isopropyl, tert-butyl,

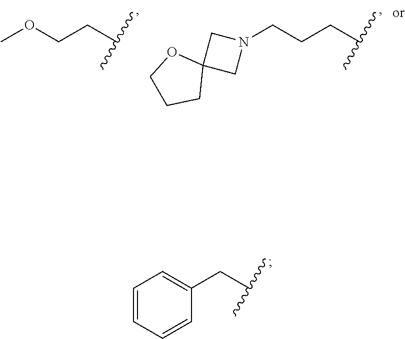

X³ is H, methyl, or ethyl; R² is isopropyl; and R¹ is

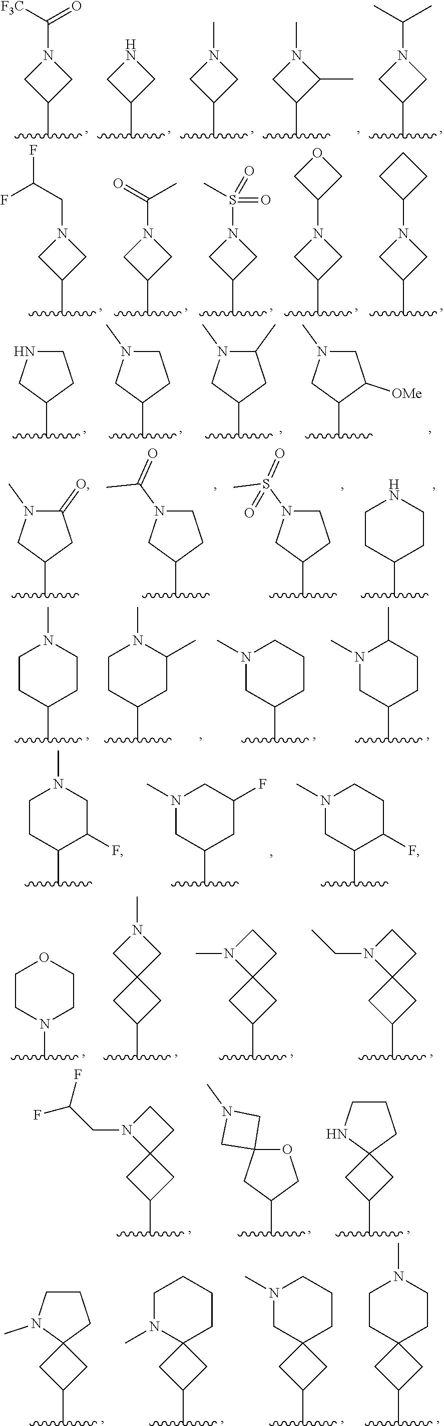

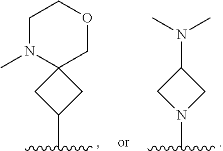

In some embodiments, W is N; Y is N; Z is O; Ring A is

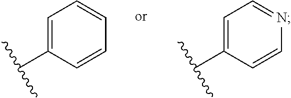 [SMILES: *c1ccccc1 ; "or" ; *c1ccncc1]

R³ is H, methyl, ethyl, isopropyl, tert-butyl,

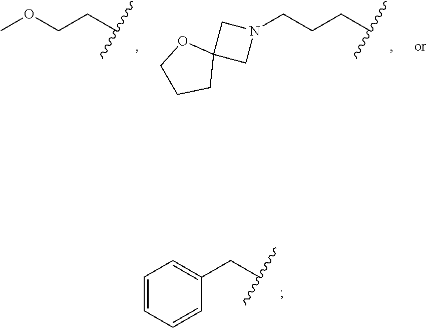

X³ is H, methyl, or ethyl; R² is isopropyl; and R¹ is

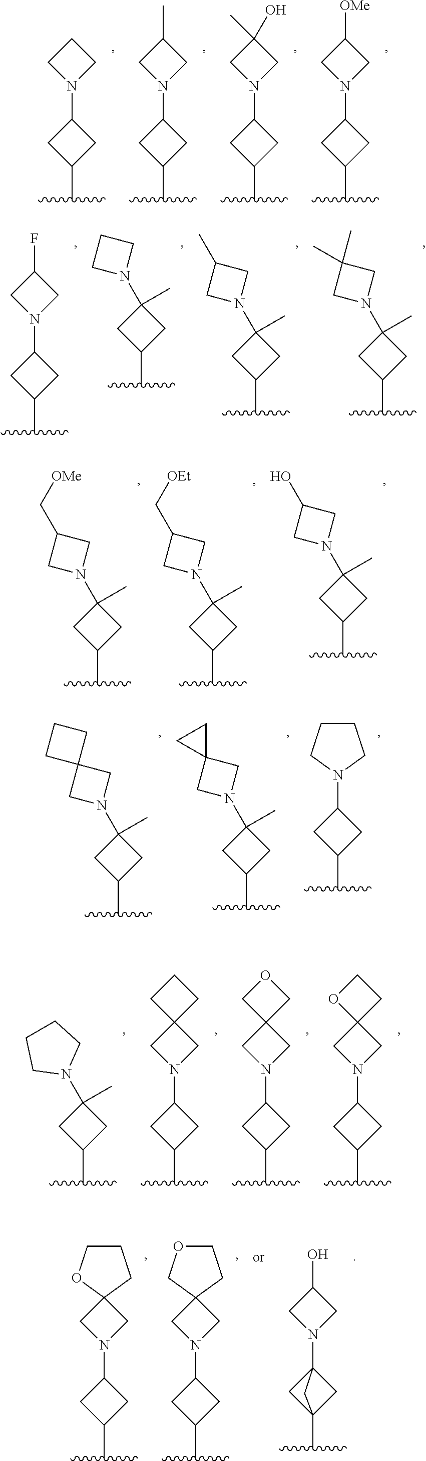

In some embodiments, W is N, Y is N; Z is O; and

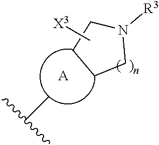

is

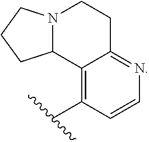 [SMILES: *c1ccnc2c1C1CCCN1CC2]

In some embodiments, W is N, Y is N; Z is O;

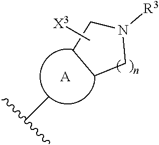

is

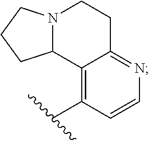 [SMILES: *c1ccnc2c1C1CCCN1CC2]

R² is isopropyl; and R¹ is

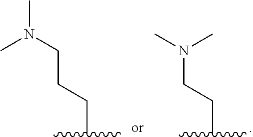

In some embodiments, W is N; Y is N; Z is O;

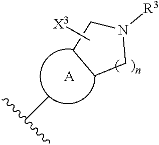

is

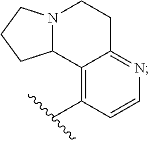 [SMILES: *c1ccnc2c1C1CCCN1CC2]

R² is isopropyl; and R¹ is

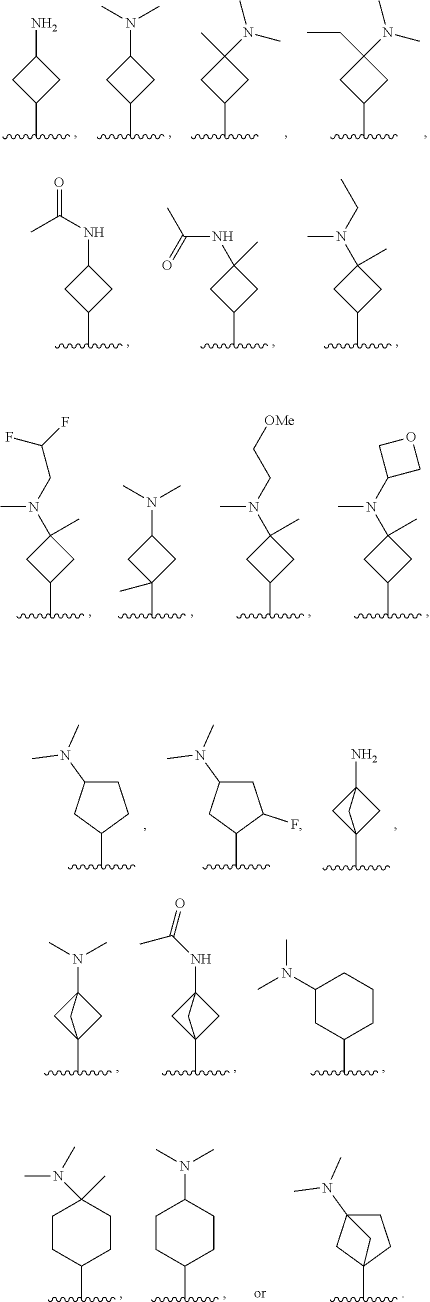

In some embodiments, W is N; Y is N, Z is O;

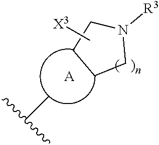

is

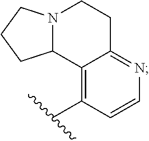 [SMILES: *c1ccnc2c1C1CCCN1CC2]

R² is isopropyl; and R¹ is

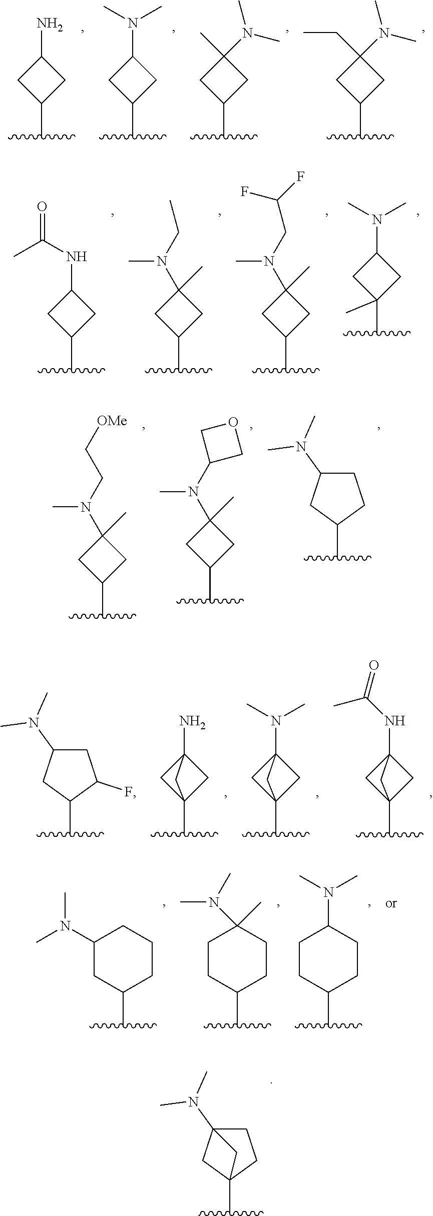

In some embodiments, W is N; Y is N; Z is O;

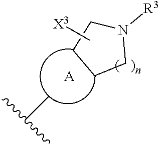

is

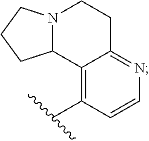 [SMILES: *c1ccnc2c1C1CCCN1CC2]

R² is isopropyl; and R¹ is

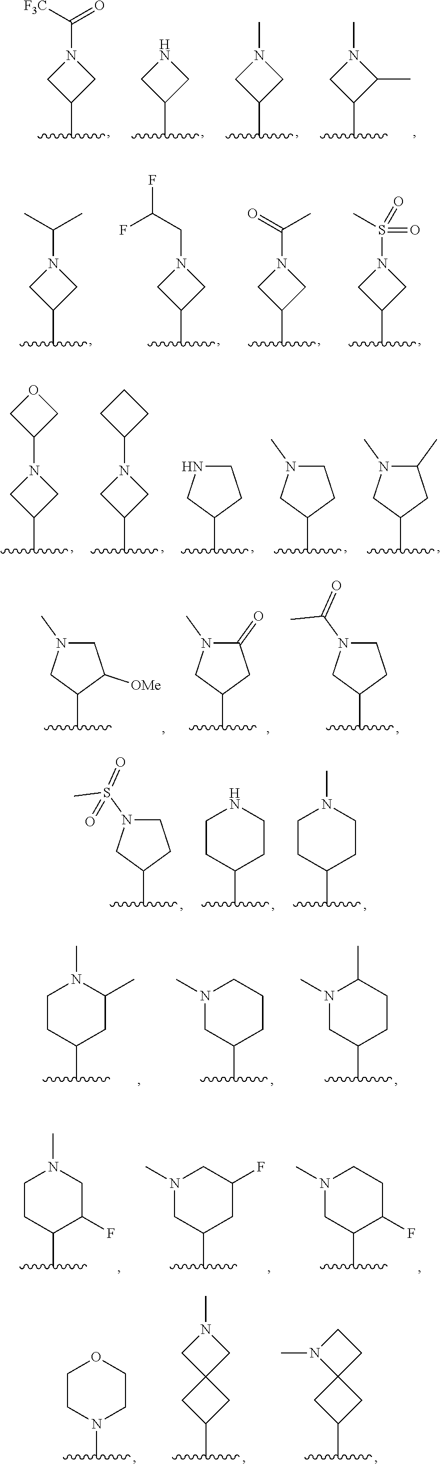

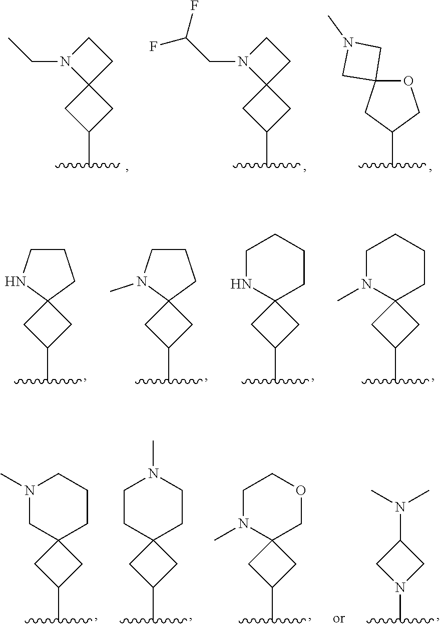

In some embodiments, W is N; Y is N; Z is O;

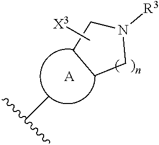

is

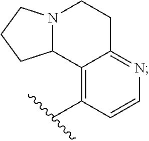 [SMILES: *c1ccnc2c1C1CCCN1CC2]

R² is isopropyl; and R¹ is

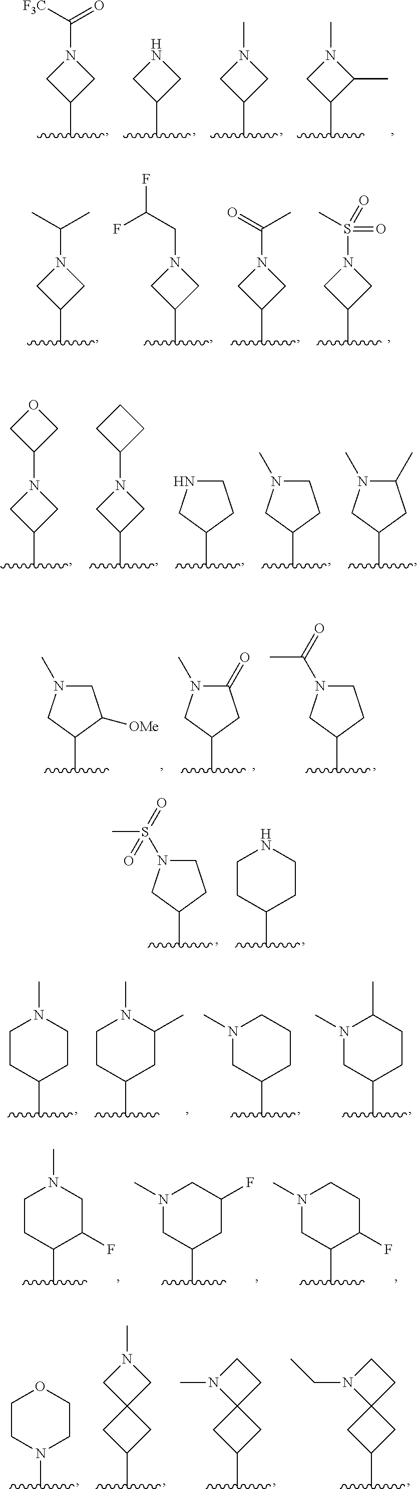

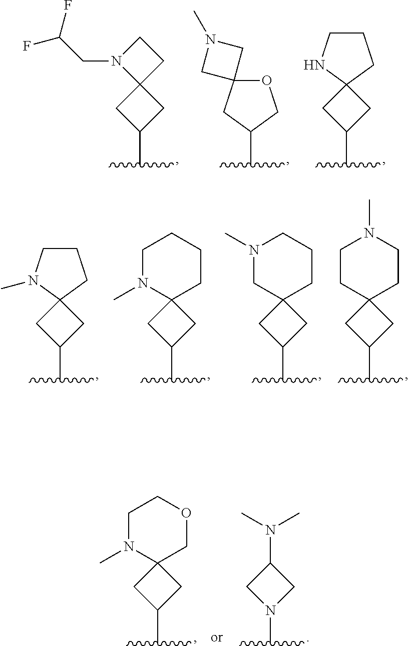

In some embodiments, W is N; Y is N; Z is O;

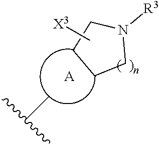

is

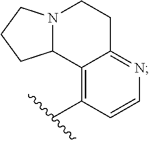 [SMILES: *c1ccnc2c1C1CCCN1CC2]

R² is isopropyl; and R¹ is

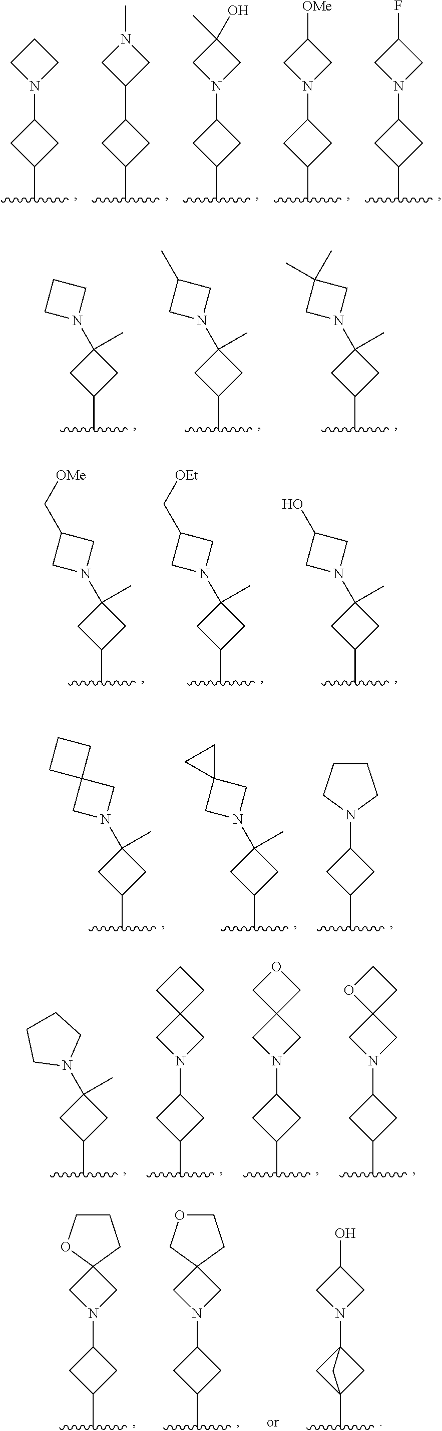

In some embodiments, W is N, Y is N, Z is O, Ring A is

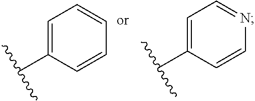 [SMILES: *c1ccccc1 ; "or" ; *c1ccncc1]

and n is 1, 2, or 3.

In some embodiments, W is N; Y is N; Z is O; Ring A is

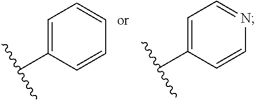 [SMILES: *c1ccccc1 ; *c1ccncc1]

R² is isopropyl; R¹ is

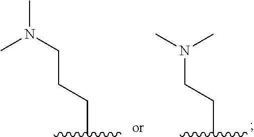

and n is 1, 2, or 3.

In some embodiments, W is N; Y is N; Z is O; Ring A is

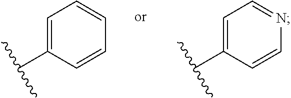 [SMILES: *c1ccccc1 ; "or" ; *c1ccncc1]

R² is isopropyl; R¹ is

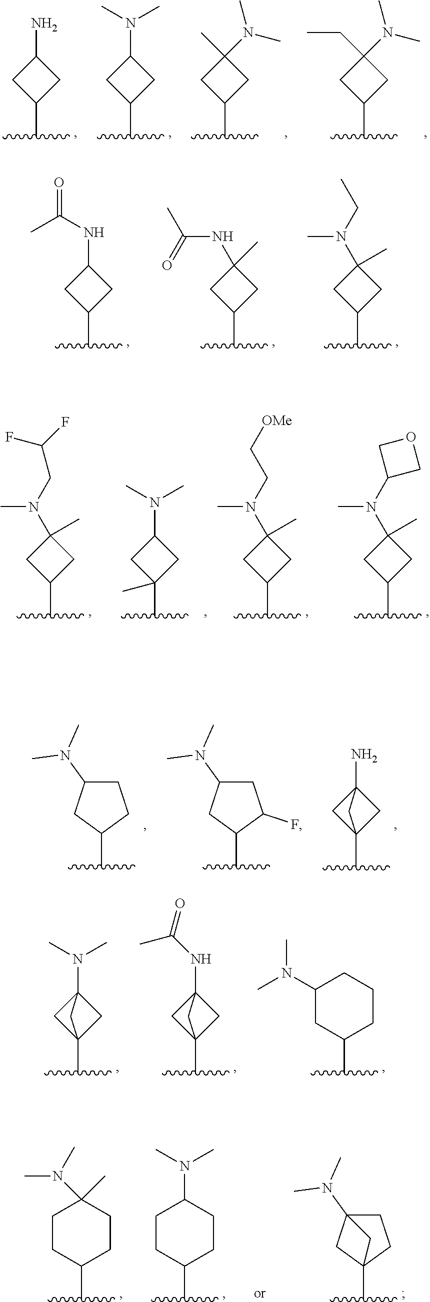

and n is 1, 2, or 3.

In some embodiments, W is N; Y is N; Z is O; Ring A is

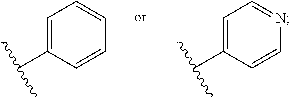 [SMILES: *c1ccccc1 ; "or" ; *c1ccncc1]

R² is isopropyl; R¹ is

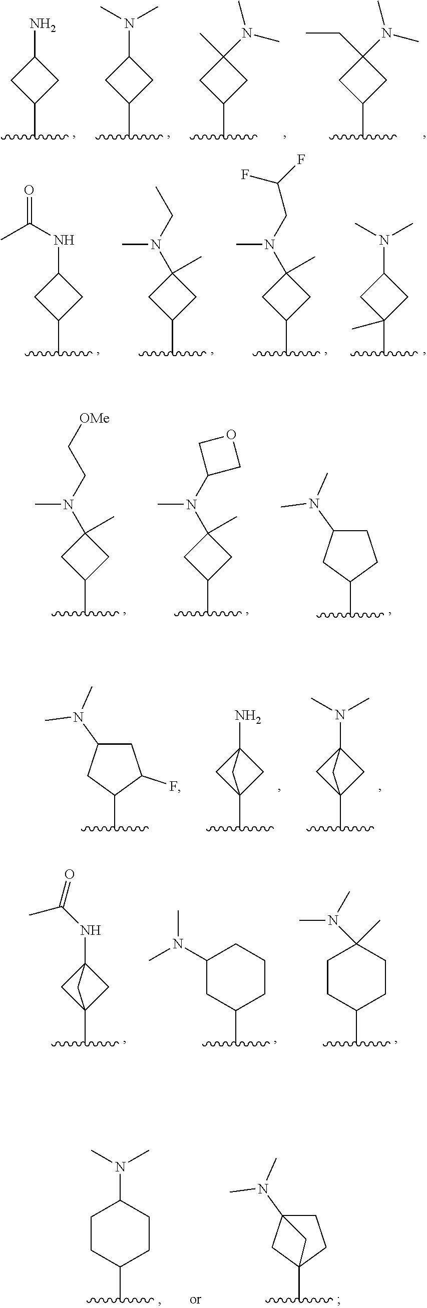

and n is 1, 2, or 3.

In some embodiments W is N; Y is N; Z is O; Ring A is

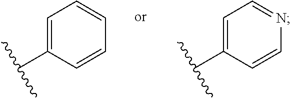 [SMILES: *c1ccccc1 ; "or" ; *c1ccncc1]

R² is isopropyl; R¹ is

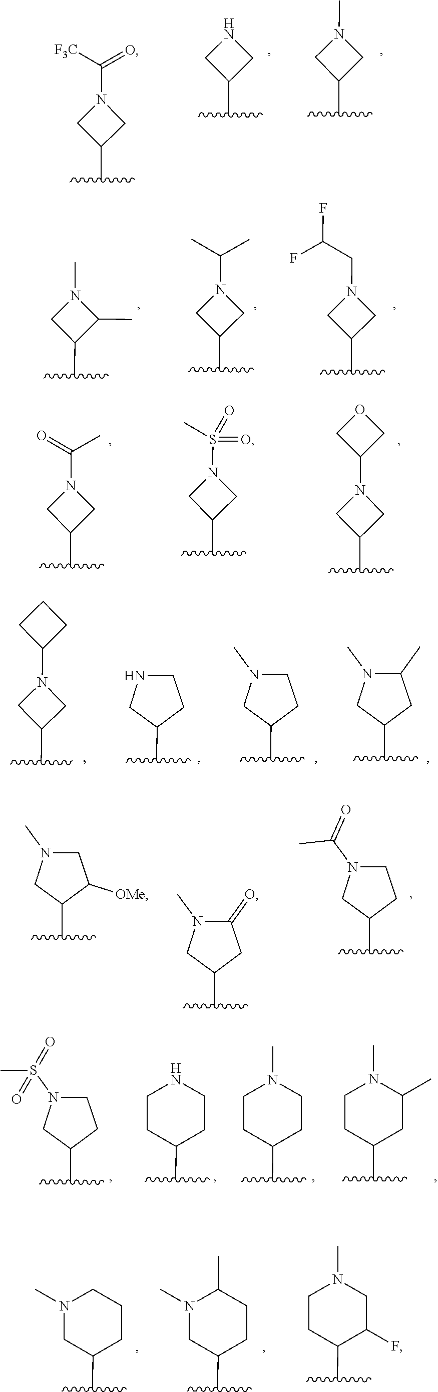

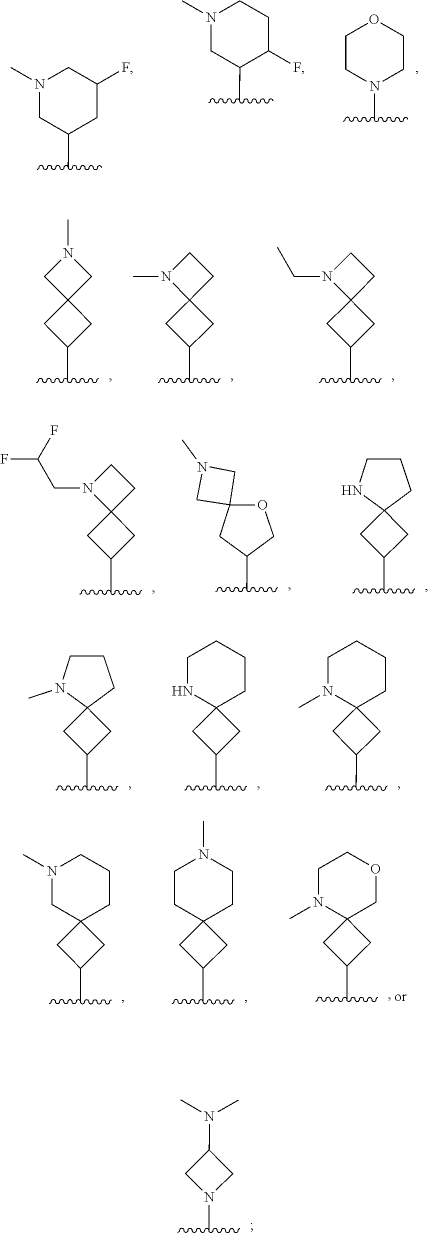

and n is 1, 2, or 3.

In some embodiments, W is N; Y is N; Z is O; Ring A is

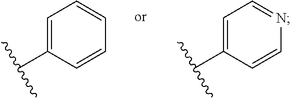 [SMILES: *c1ccccc1 ; "or" ; *c1ccncc1]

R² is isopropyl; R¹ is

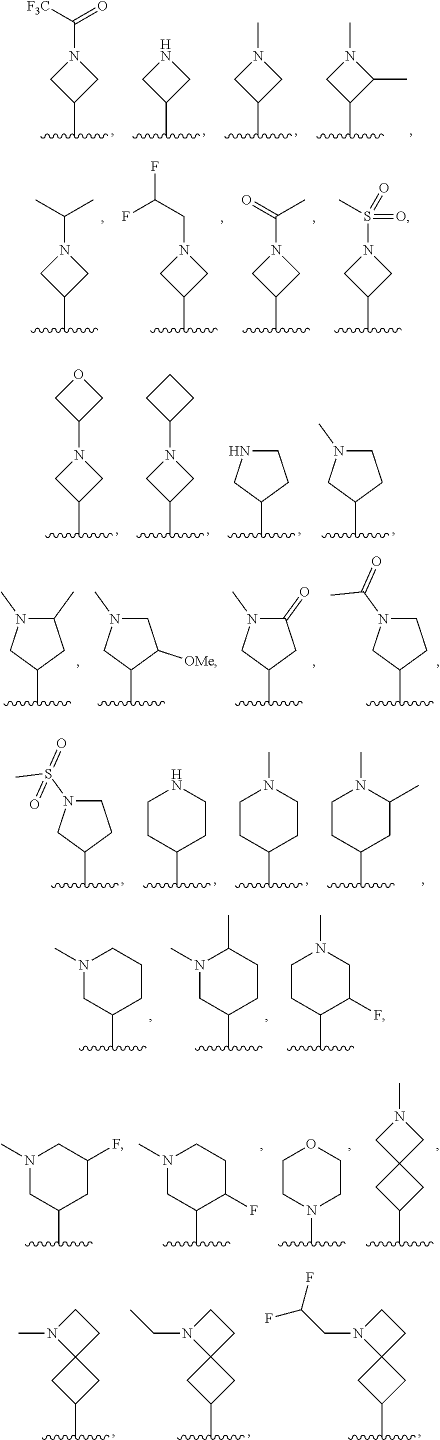

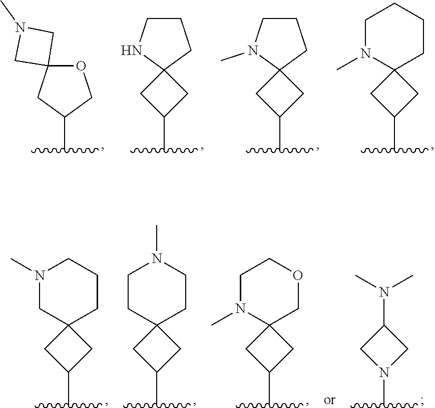

and n is 1, 2, or 3.

In some embodiments, W is N; Y is N; Z is O; Ring A is

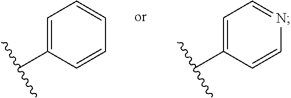 [SMILES: *c1ccccc1 ; "or" ; *c1ccncc1]

R² is isopropyl; R¹ is

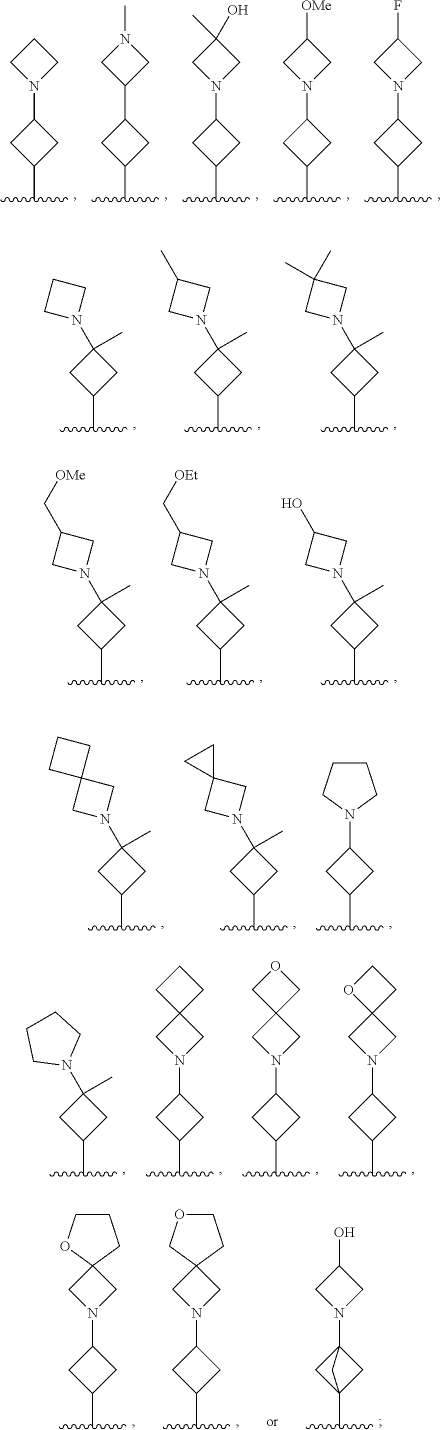

and n is 1, 2, or 3.

In some embodiments, W is N; Y is N; Z is O; Ring A is

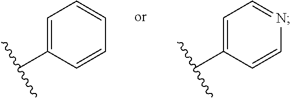 [SMILES: *c1ccccc1 ; "or" ; *c1ccncc1]

X³ is H, methyl, or ethyl; and n is 1, 2, or 3.

In some embodiments, W is N; Y is N; Z is O; Ring A is

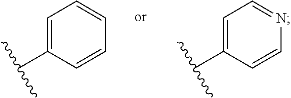 [SMILES: *c1ccccc1 ; "or" ; *c1ccncc1]

X³ is H, methyl, or ethyl; R² is isopropyl; R¹ is

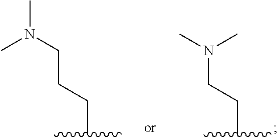

and n is 1, 2, or 3.

In some embodiments, W is N; Y is N; Z is O; Ring A is

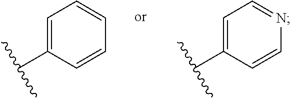 [SMILES: *c1ccccc1 ; "or" ; *c1ccncc1]

X³ is H, methyl, or ethyl; R² is isopropyl; R¹ is

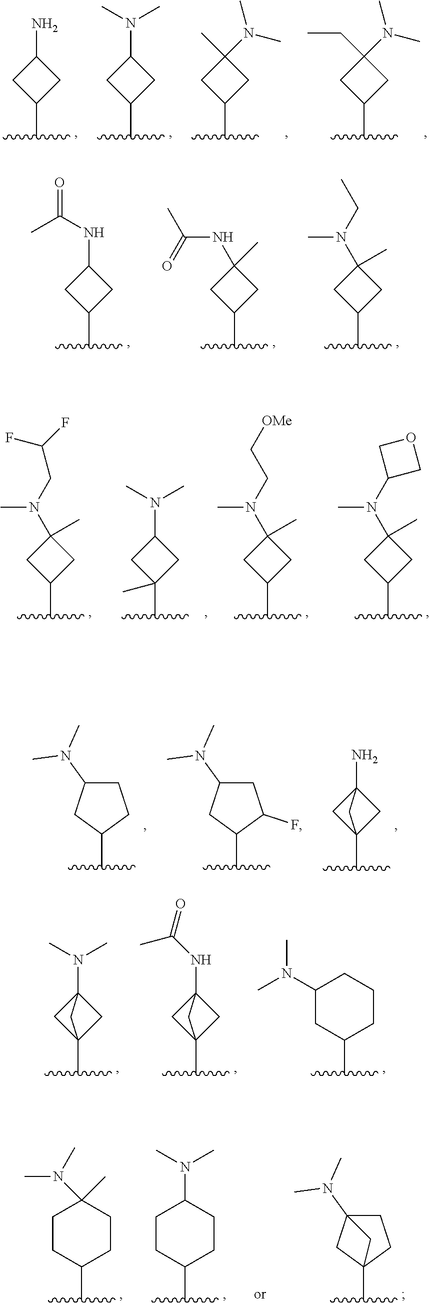

and n is 1, 2, or 3.

In some embodiments, W is N; Y is N; Z is O; Ring A is

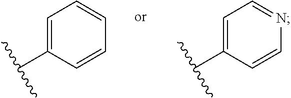 [SMILES: *c1ccccc1 ; "or" ; *c1ccncc1]

X³ is H, methyl, or ethyl; R² is isopropyl; R¹ is

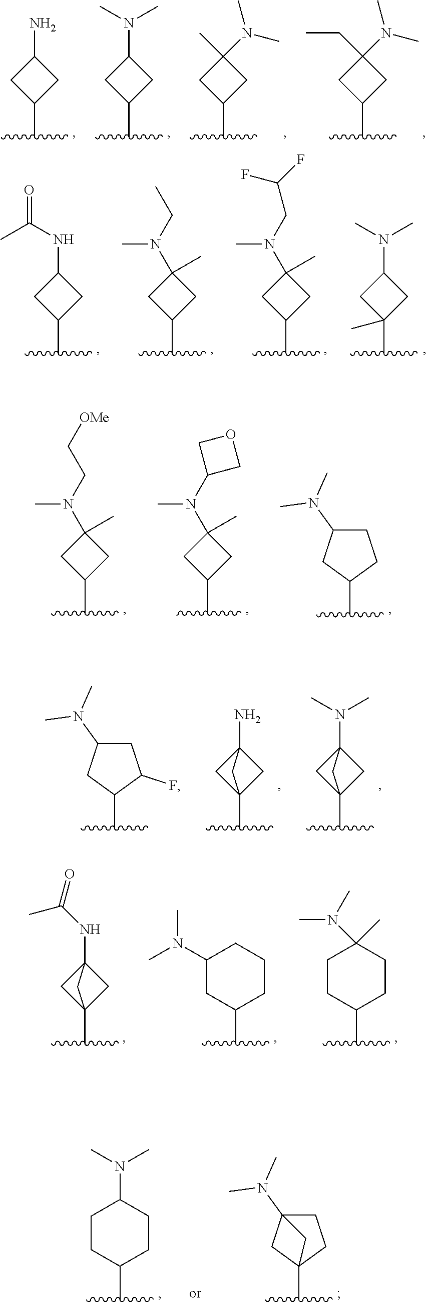

and n is 1, 2, or 3.

In some embodiments, W is N; Y is N; Z is O, Ring A is

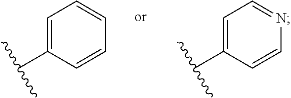 [SMILES: *c1ccccc1 ; "or" ; *c1ccncc1]

X³ is H, methyl, or ethyl; R² is isopropyl; R¹ is

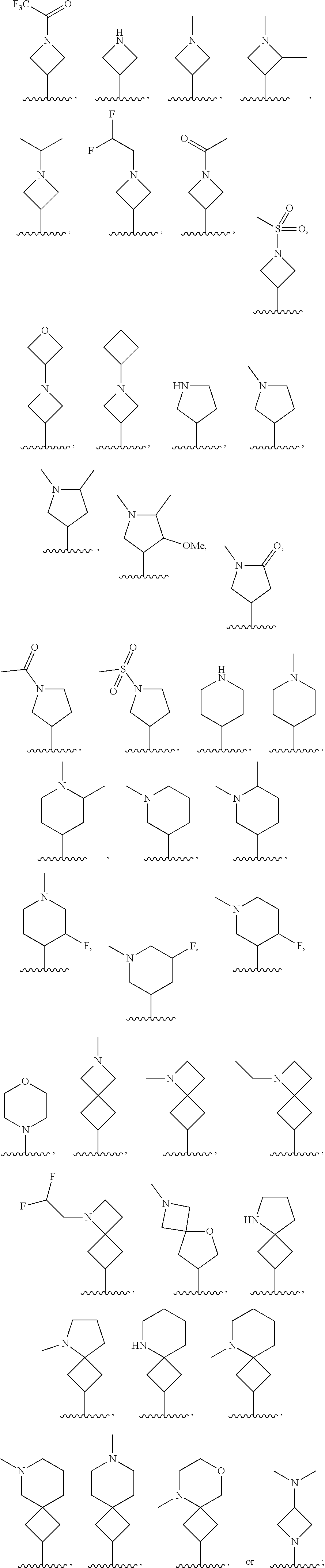

and n is 1, 2, or 3.

In some embodiments, W is N, Y is N; Z is O, Ring A is

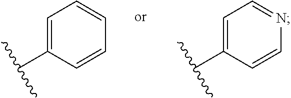 [SMILES: *c1ccccc1 ; "or" ; *c1ccncc1]

X³ is H, methyl, or ethyl; R² is isopropyl; R¹ is

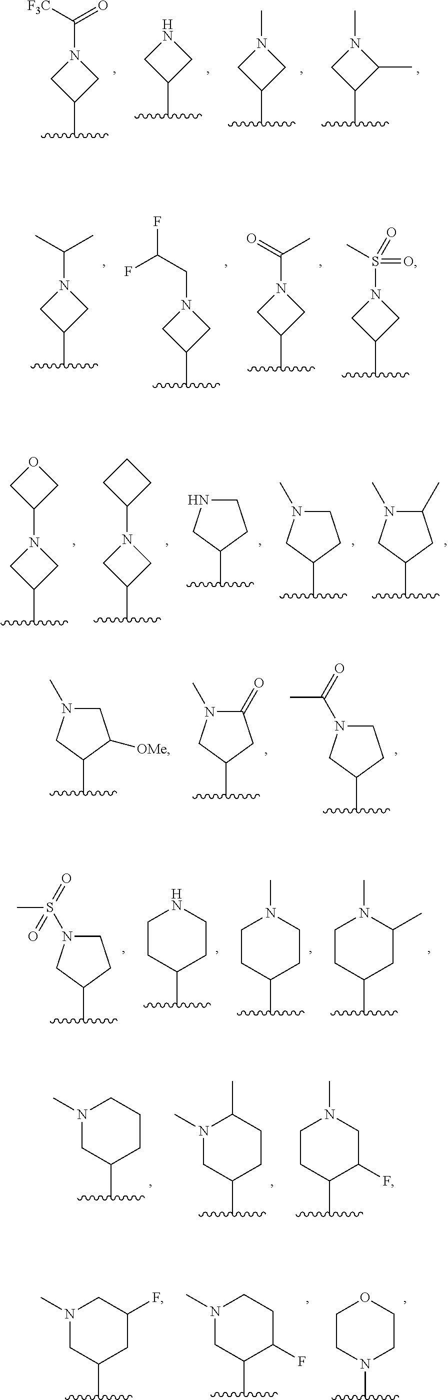

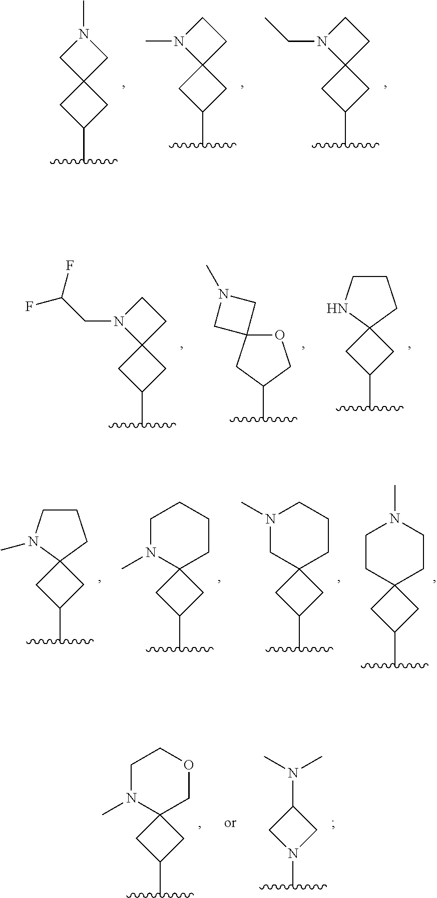

and n is 1, 2, or 3.1

In some embodiments, W is N; Y is N; Z is O; Ring A is

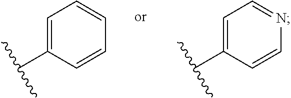 [SMILES: *c1ccccc1 ; "or" ; *c1ccncc1]

X³ is HK methyl, or ethyl; R² is isopropyl; R¹ is

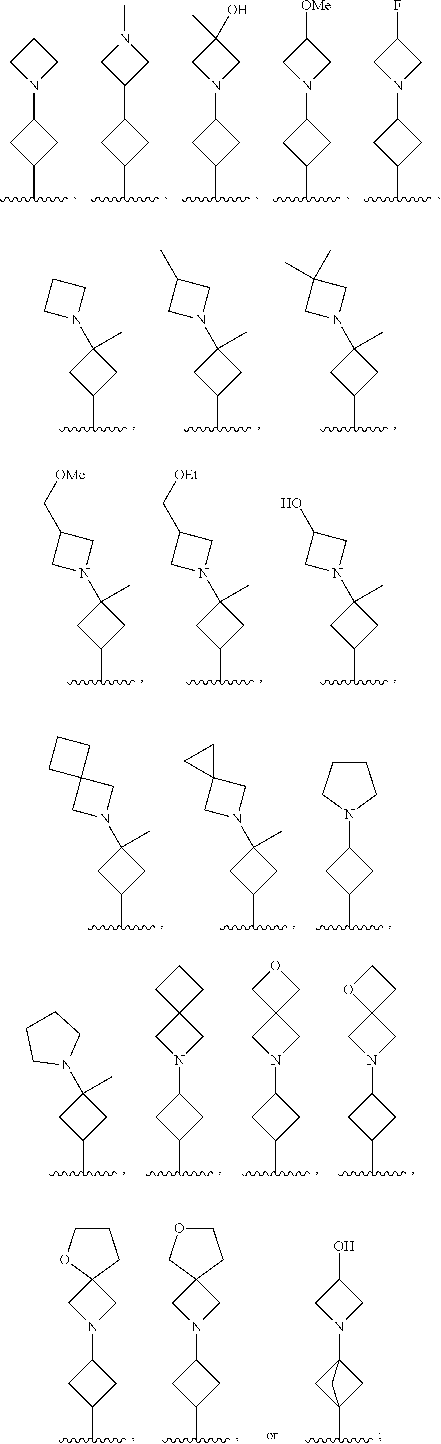

and n is 1, 2 or 3.

In some embodiments, W is N; Y is N; Z is O; Ring A is

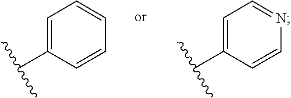 [SMILES: *c1ccccc1 ; "or" ; *c1ccncc1]

R³ is H, methyl, ethyl, isopropyl, tert-butyl,

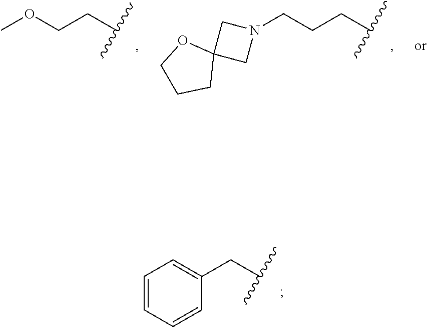

X³ is H, methyl, or ethyl; and n is 1, 2, or 3.

In some embodiments, W is N; Y is N; Z is O; Ring A is

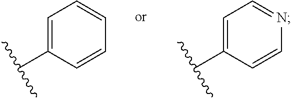 [SMILES: *c1ccccc1 ; "or" ; *c1ccncc1]

R³ is H, methyl, ethyl, isopropyl, tert-butyl,

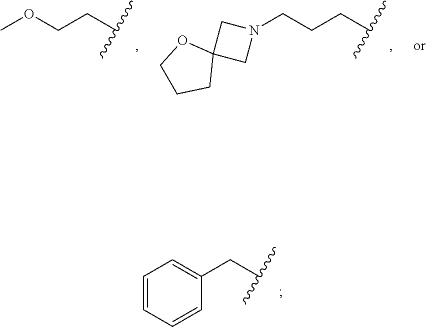

X³ is H, methyl, or ethyl; R² is isopropyl; R¹ is

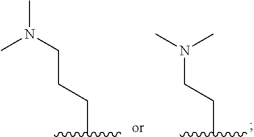

and n is 1, 2, or 3.

In some embodiments, W is N; Y is N; Z is O; Ring A is

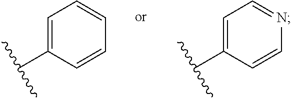 [SMILES: *c1ccccc1 ; "or" ; *c1ccncc1]

R³ is H, methyl, ethyl, isopropyl, tert-butyl,

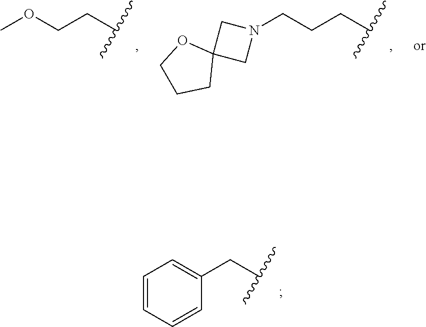

X³ is H, methyl, or ethyl; R² is isopropyl; R¹ is

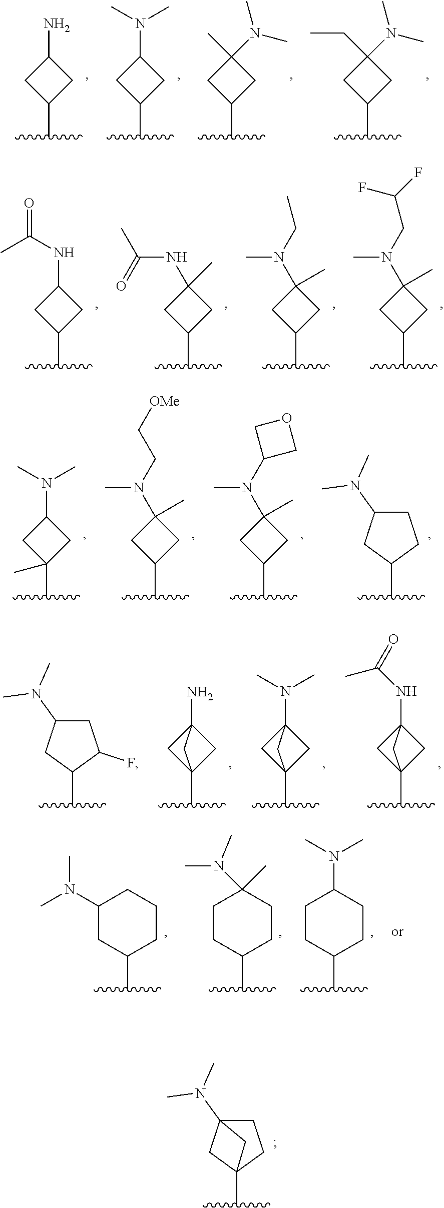

and n is 1, 2, or 3.

In some embodiments, W is N; Y is N; Z is O; Ring A is

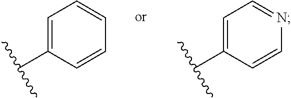 [SMILES: *c1ccccc1 ; "or" ; *c1ccncc1]

R³ is H, methyl, ethyl, isopropyl, tert-butyl,

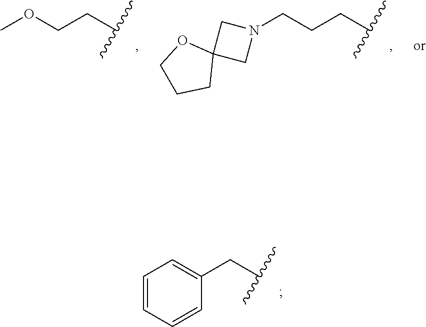

X³ is H, methyl, or ethyl; R² is isopropyl; R¹ is

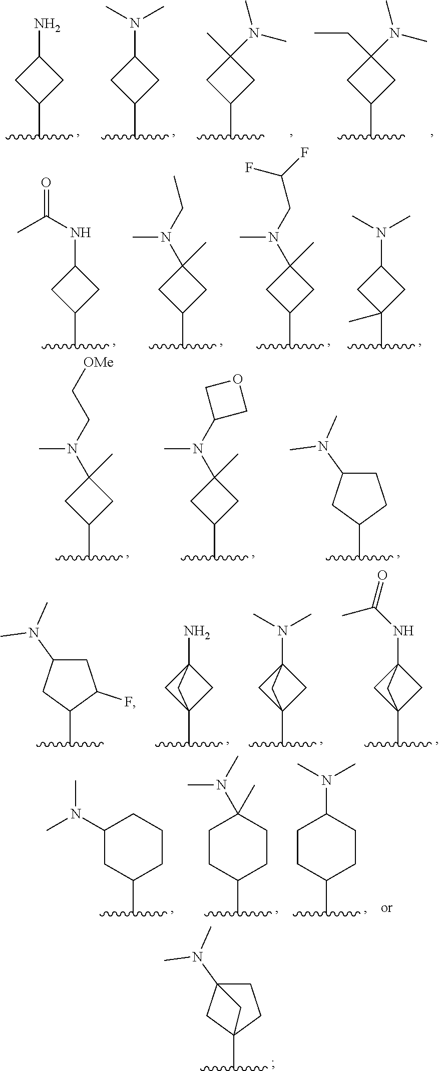

and n is 1, 2 or 3.

In some embodiments, W is N; Y is N; Z is O; Ring A is

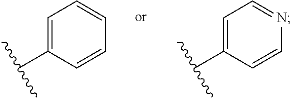 [SMILES: *c1ccccc1 ; "or" ; *c1ccncc1]

R³ is H, methyl, ethyl, isopropyl, tert-butyl,

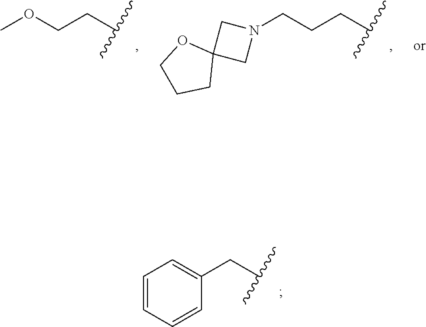

X³ or is H, methyl, or ethyl; R² is isopropyl; R¹ is

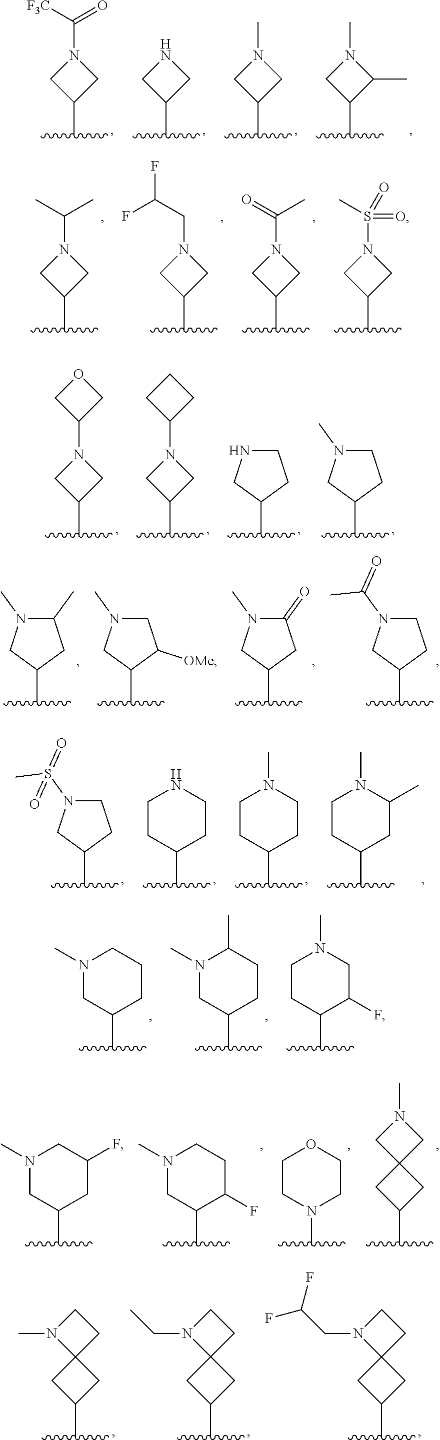

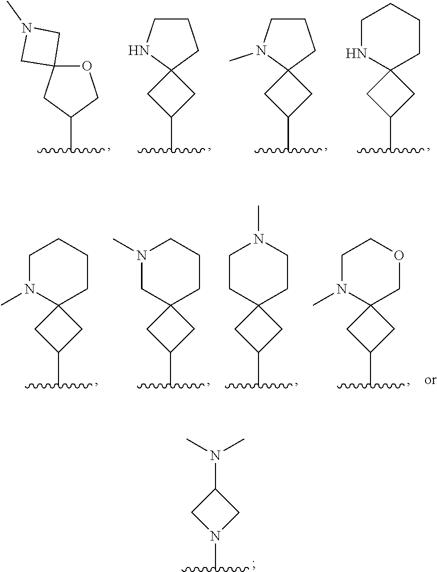

and n is 1, 2, or 3.

In some embodiments, W is N; Y is N; Z is O; Ring A is

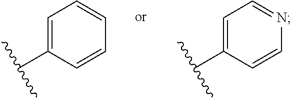 [SMILES: *c1ccccc1 ; "or" ; *c1ccncc1]

R³ is H methyl, ethyl, isopropyl, tert-butyl,

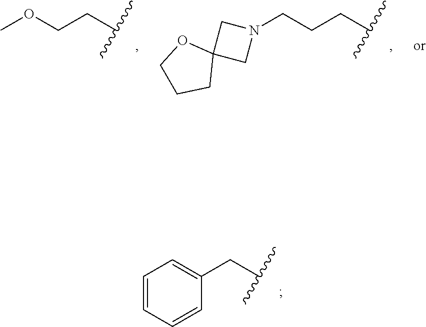

X³ is H, methyl, or ethyl; R² is isopropyl; R¹ is

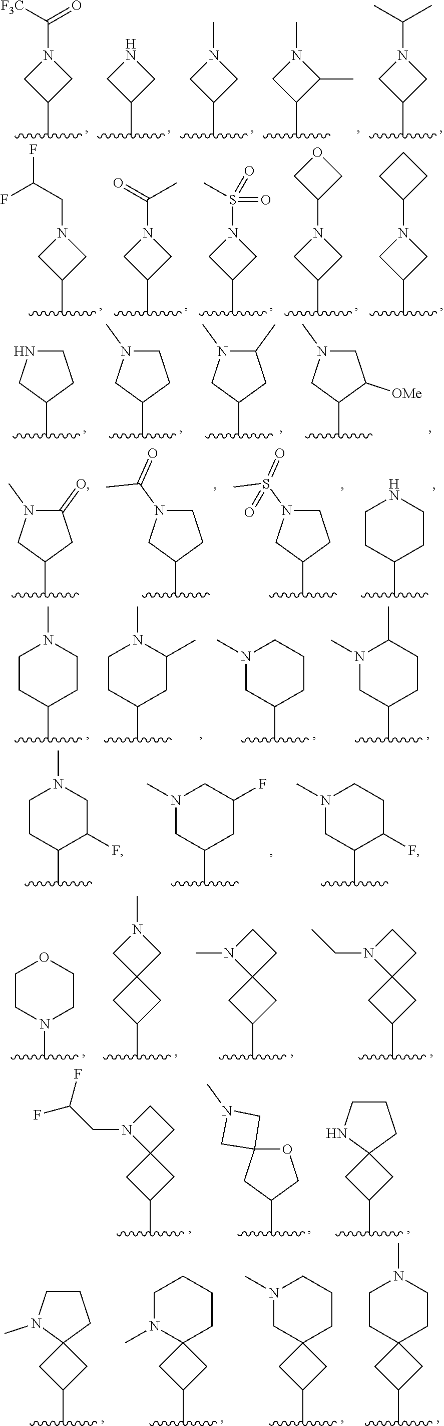

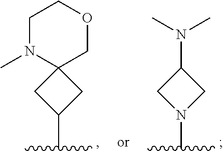

and n is 1, 2, or 3.

In some embodiments, W is N; Y is N; Z is O; Ring A is

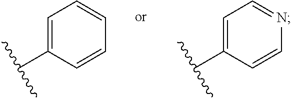 [SMILES: *c1ccccc1 ; "or" ; *c1ccncc1]

R³ is H, methyl, ethyl, isopropyl, tert-butyl,

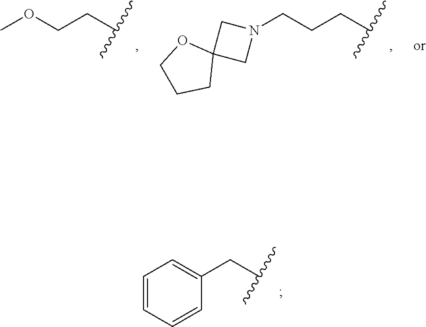

X³ is H, methyl, or ethyl; R² is isopropyl; R¹ is

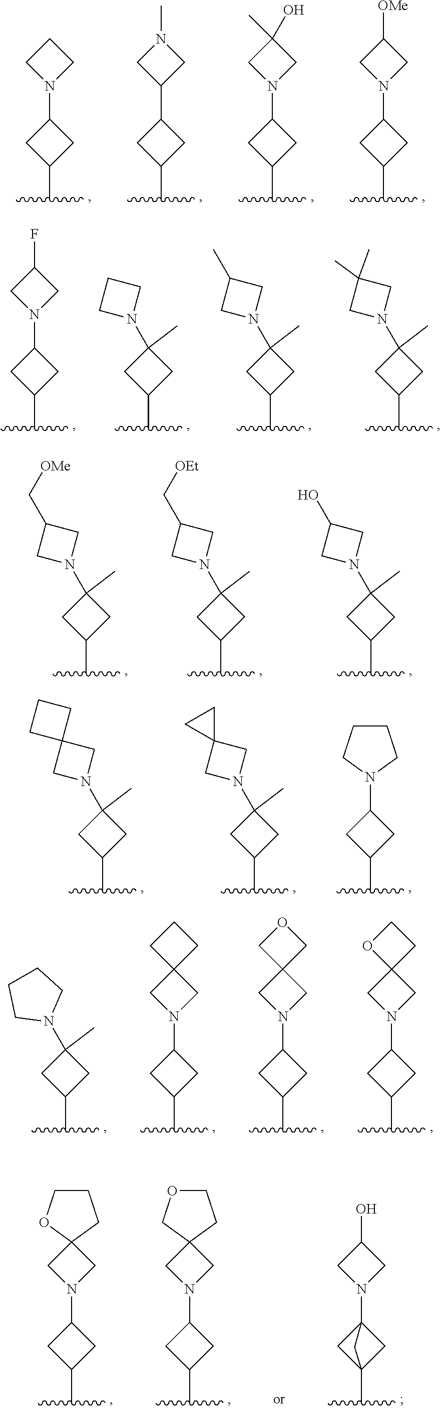

and n is 1, 2, or 3.

In some embodiments, W is N, Y is N, Z is O,

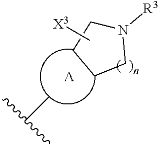

is

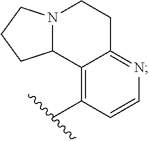 [SMILES: *c1ccnc2c1C1CCCN1CC2]

and n is 1, 2, or 3.

In some embodiments, W is N; Y is N; Z is O;

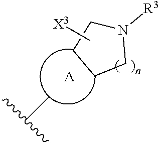

is

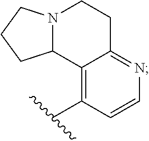 [SMILES: *c1ccnc2c1C1CCCN1CC2]

R² is isopropyl; R¹ is

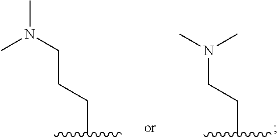

and n is 1, 2, or 3.

In some embodiments, W is N, Y is N; Z is O;

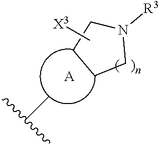

is

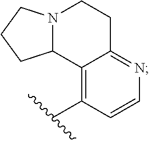 [SMILES: *c1ccnc2c1C1CCCN1CC2]

R² is isopropyl; R¹ is

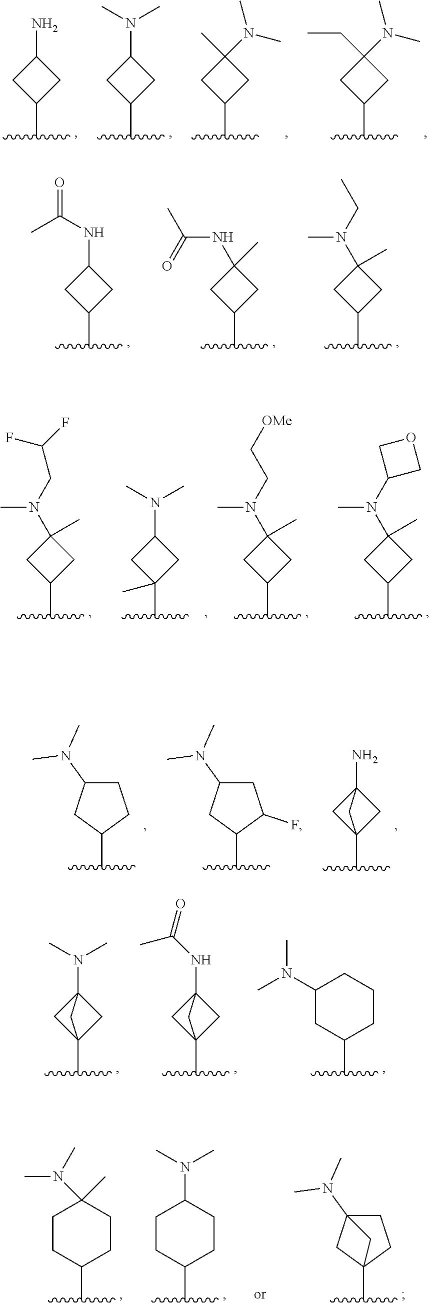

and n is 1, 2, or 3.

In some embodiments, W is N; Y is N; Z is O; is

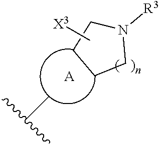

is

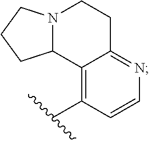 [SMILES: *c1ccnc2c1C1CCCN1CC2]

R² is isopropyl; R¹ is

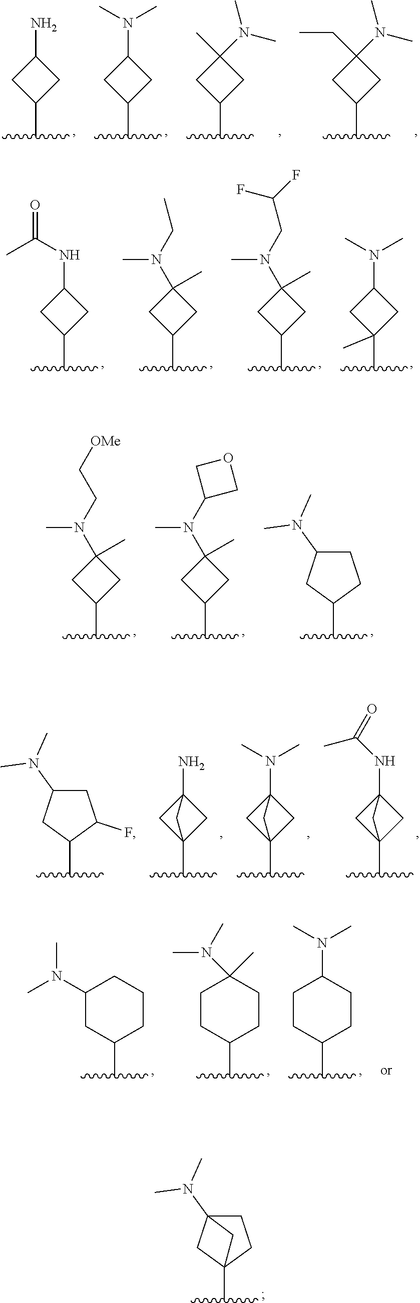

and n is 1, 2, or 3.

In some embodiments, W is N, Y is N; Z is O;

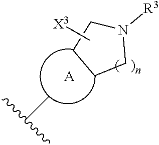

is

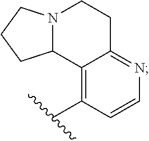 [SMILES: *c1ccnc2c1C1CCCN1CC2]

R² is isopropyl; R¹ is

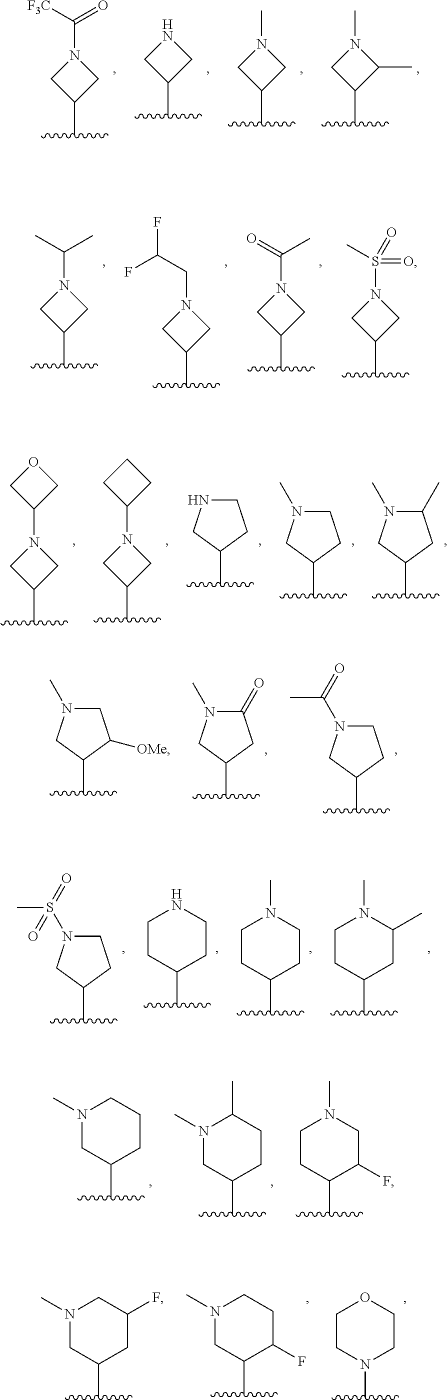

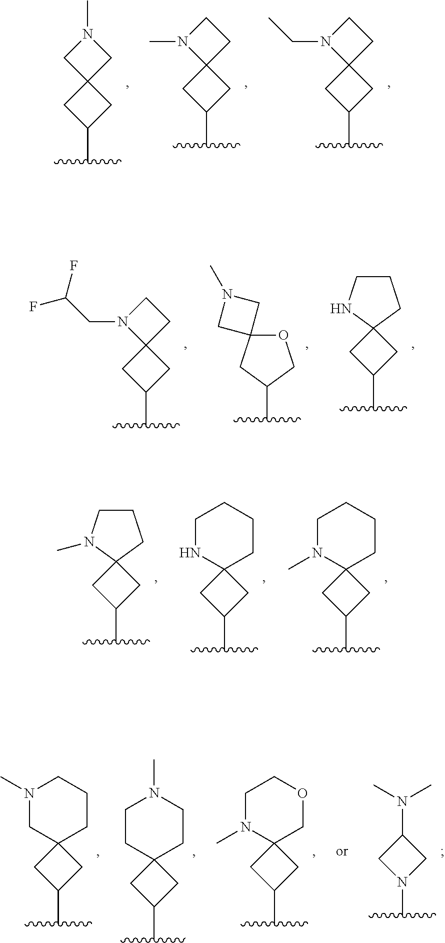

and n is 1, 2, or 3.

In some embodiments, W is N; Y is N; Z is O;

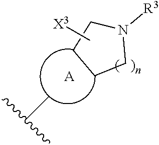

is

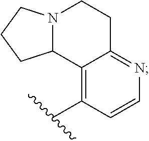 [SMILES: *c1ccnc2c1C1CCCN1CC2]

R² is isopropyl; R¹ is

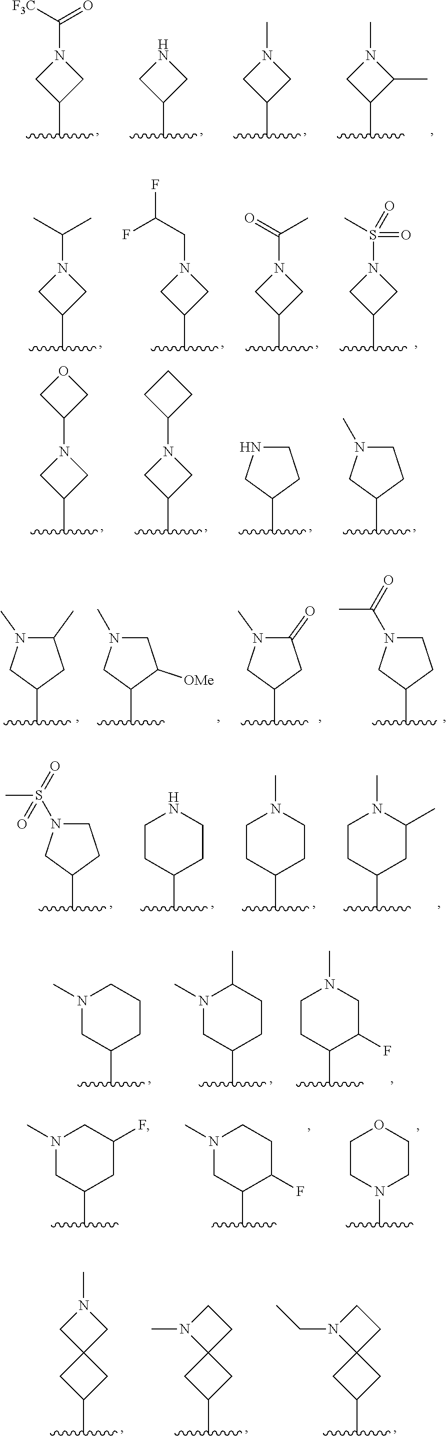

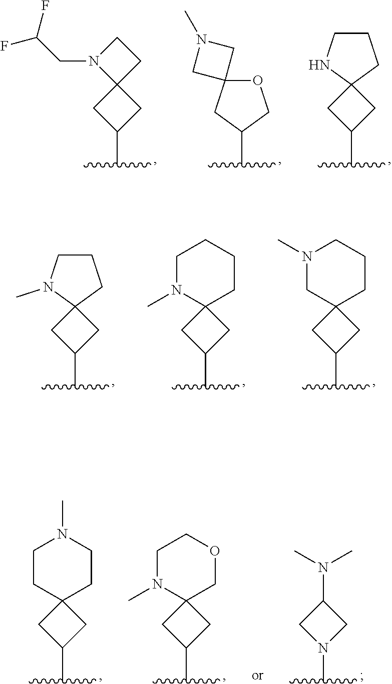

and n is 1, 2, or 3.

In some embodiments, W is N; Y is N; Z is O;

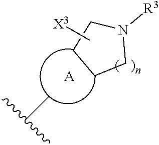

is

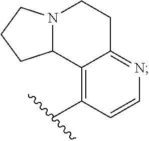 [SMILES: *c1ccnc2c1C1CCCN1CC2]

isopropyl; R¹ is

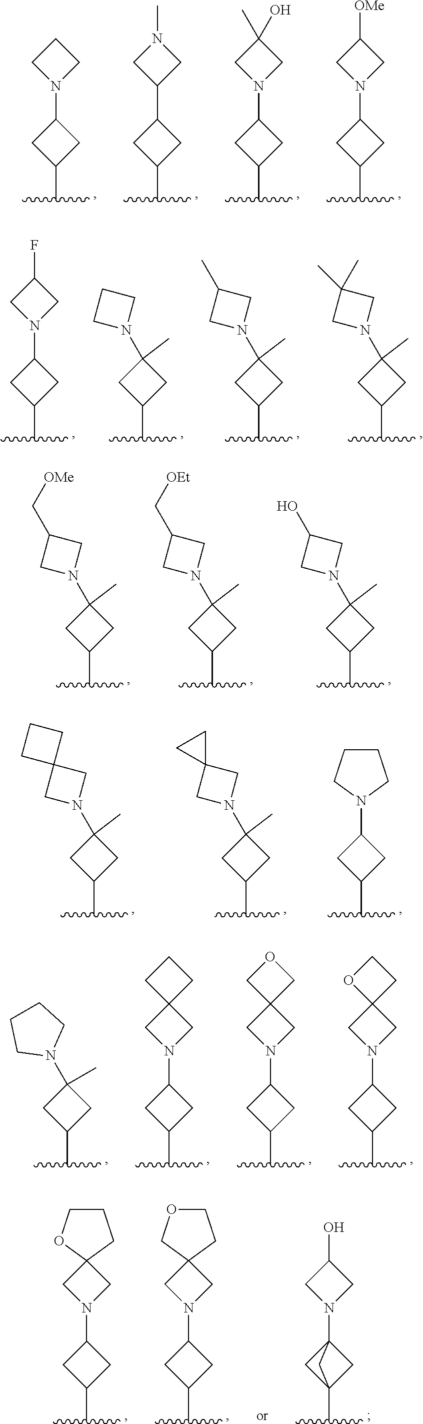

and n is 1, 2, or 3.

In some embodiments, W is N, Y is CH, Z is O, and Ring A is

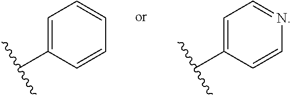 [SMILES: *c1ccccc1 ; "or" ; *c1ccncc1]

In some embodiments, W is N; Y is CH; Z is O; Ring A is

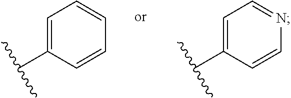 [SMILES: *c1ccccc1 ; "or" ; *c1ccncc1]

R² is isopropyl; and R¹ is

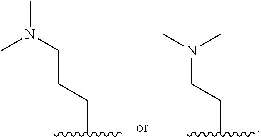

In some embodiments, W is N; Y is CH; Z is O; Ring A is

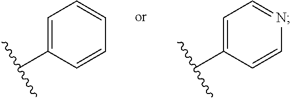 [SMILES: *c1ccccc1 ; "or" ; *c1ccncc1]

R² is isopropyl; and R¹ is

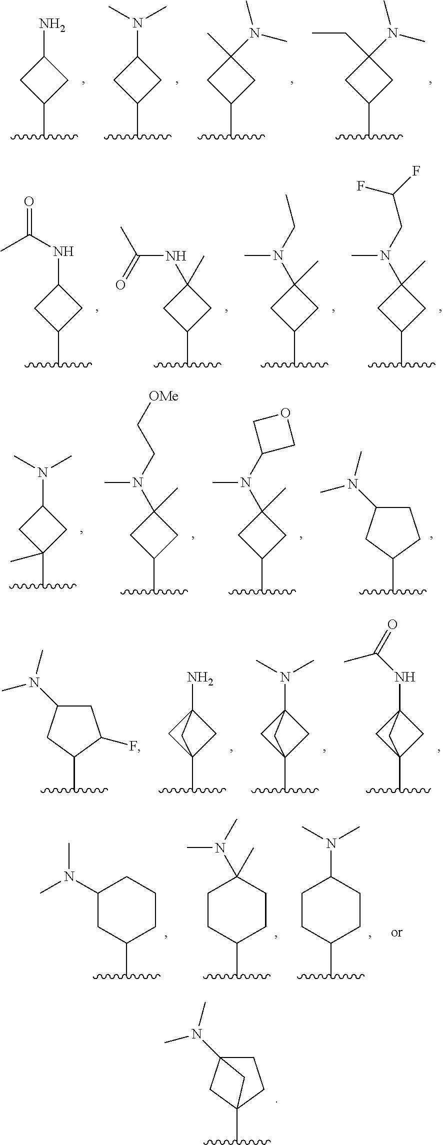

In some embodiments, W is N; Y is CH; Z is O; Ring A is

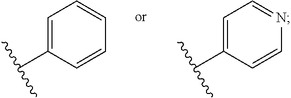 [SMILES: *c1ccccc1 ; "or" ; *c1ccncc1]

R² is isopropyl; and R¹ is

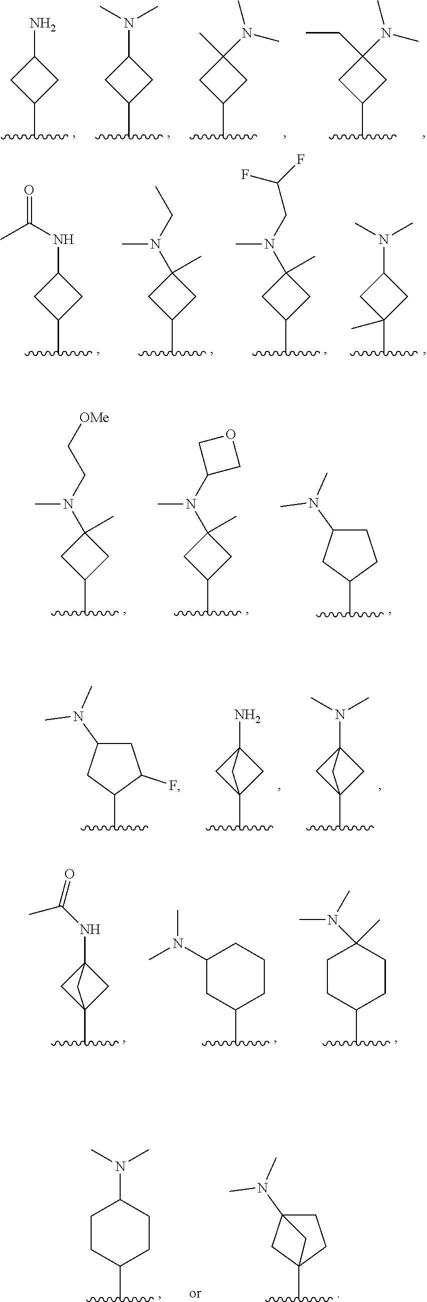

In some embodiments, W is N; Y is CH; Z is O; Ring A is

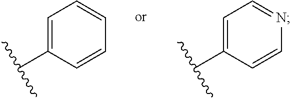 [SMILES: *c1ccccc1 ; "or" ; *c1ccncc1]

R² is isopropyl; and R¹ is

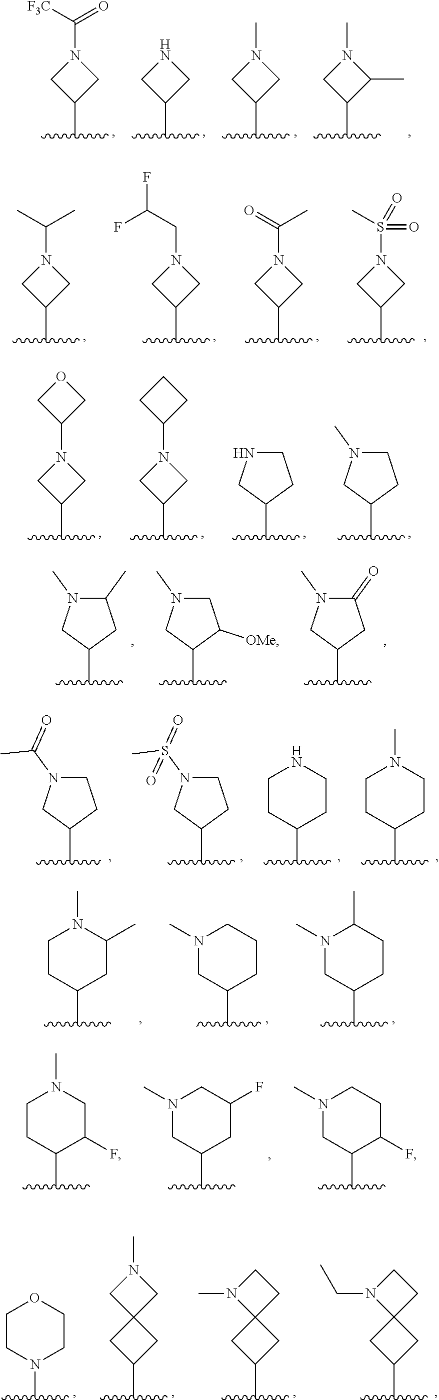

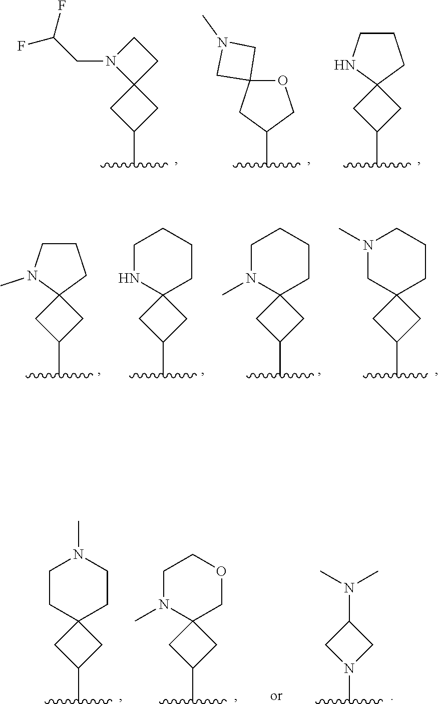

In some embodiments, W is N; Y is CH; Z is O; Ring A is

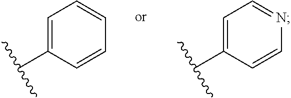 [SMILES: *c1ccccc1 ; "or" ; *c1ccncc1]

R² is isopropyl; and R¹ is

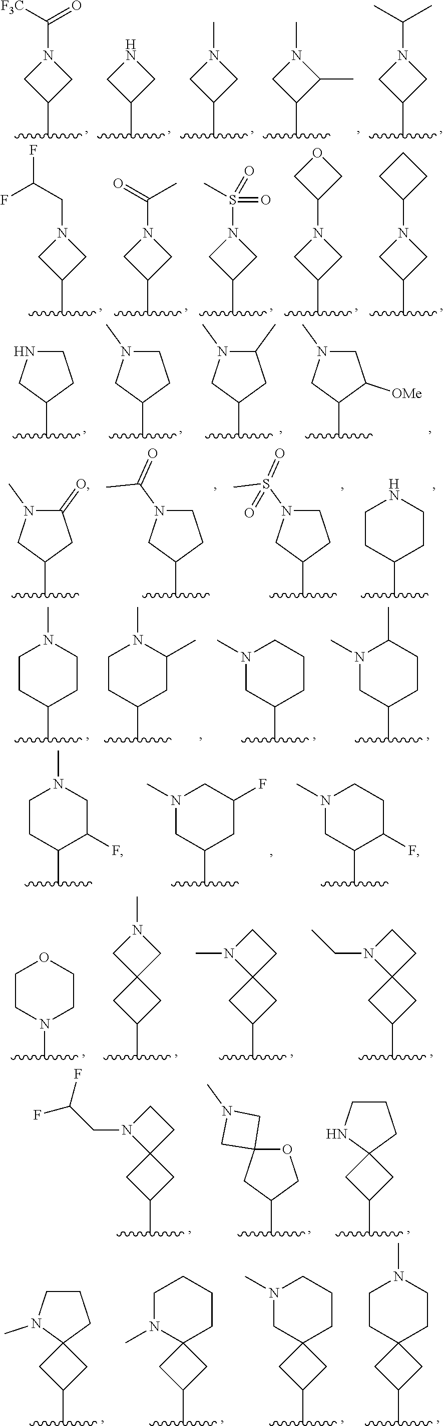

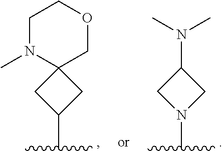

In some embodiments, W is N; Y is CH; Z is O; Ring A is

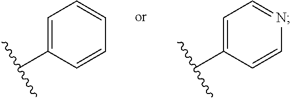 [SMILES: *c1ccccc1 ; "or" ; *c1ccncc1]

R² is isopropyl; and R¹ is

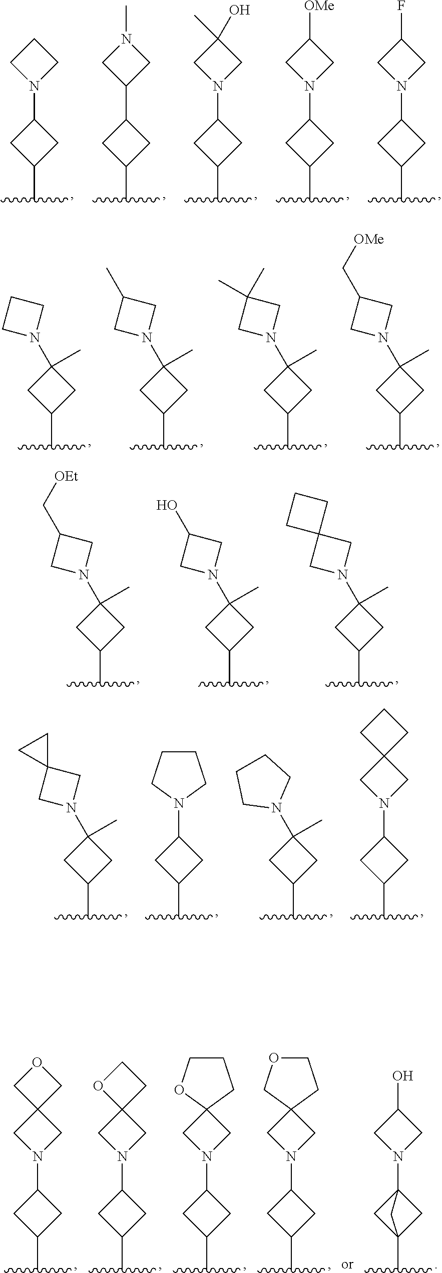

In some embodiments, W is N; Y is CH; Z is O; Ring A is

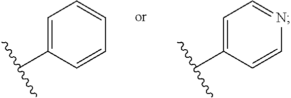 [SMILES: *c1ccccc1 ; "or" ; *c1ccncc1]

and X³ is H, methyl, or ethyl.

In some embodiments, W is N; Y is CH; Z is O; Ring A is

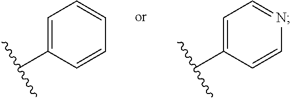 [SMILES: *c1ccccc1 ; "or" ; *c1ccncc1]

X³ is H, methyl, or ethyl; R² is isopropyl; and R¹ is

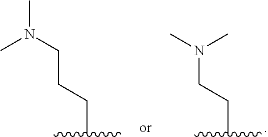

In some embodiments, W is N; Y is CH; Z is O; Ring A is

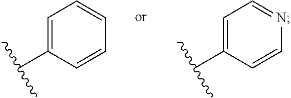 [SMILES: *c1ccccc1 ; "or" ; *c1ccncc1]

X³ is H, methyl, or ethyl; R² is isopropyl; and R¹ is

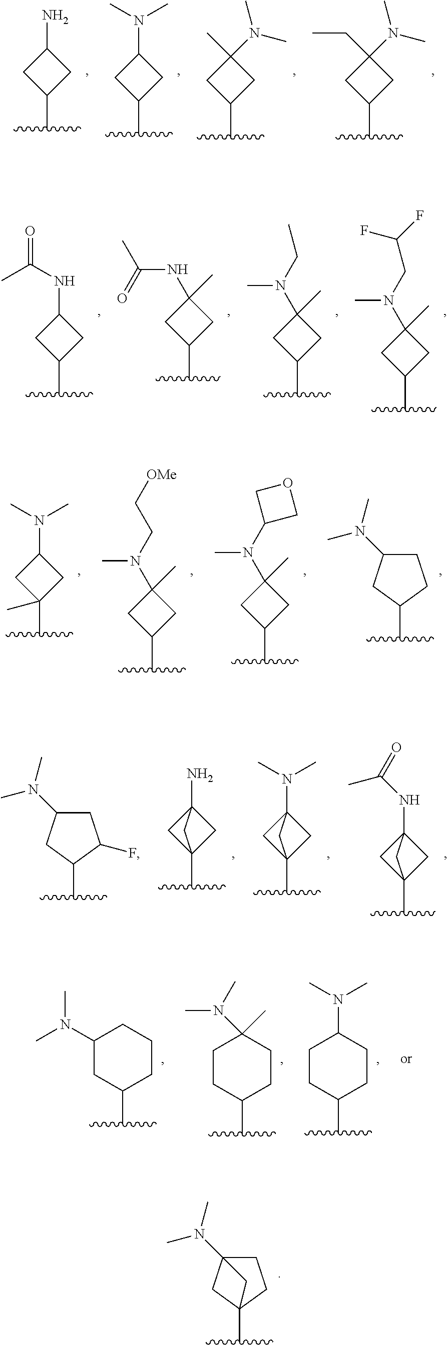

In some embodiments, W is N; Y is CH; Z is O; Ring A is

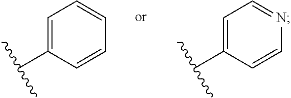 [SMILES: *c1ccccc1 ; "or" ; *c1ccncc1]

X³ is H, methyl, or ethyl, R² is isopropyl; and R¹ is

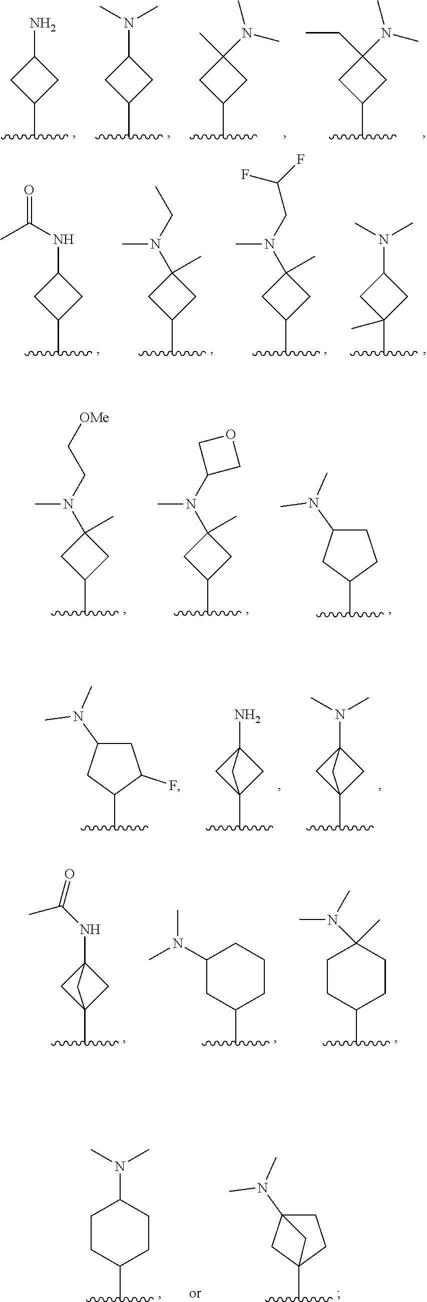

In some embodiments, W is N; Y is CH; Z is O; Ring A is

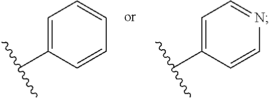 [SMILES: *c1ccccc1 ; "or" ; *c1ccncc1]

X³ is H, methyl, or ethyl; R² is isopropyl; and R¹ is

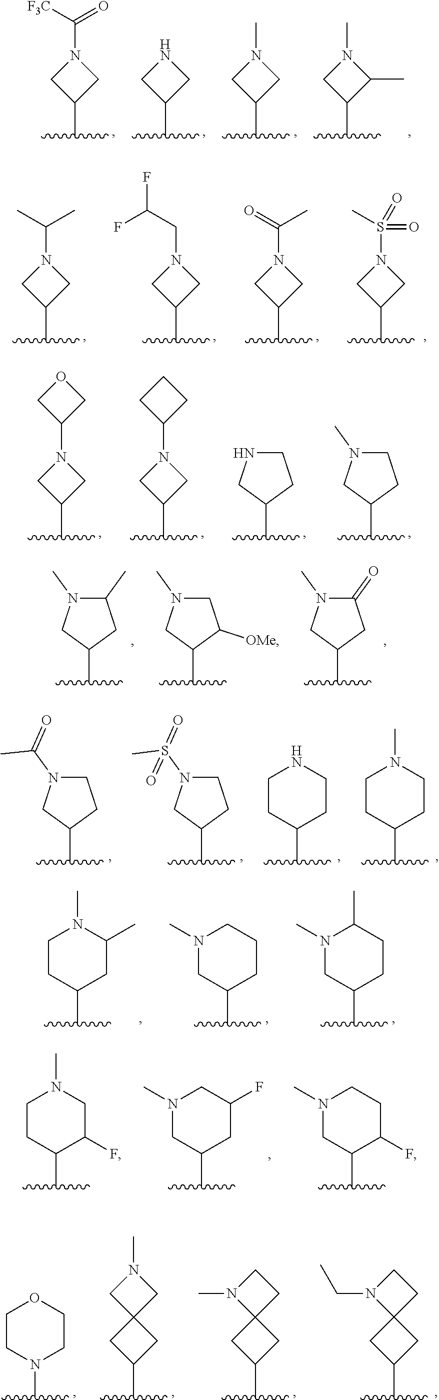

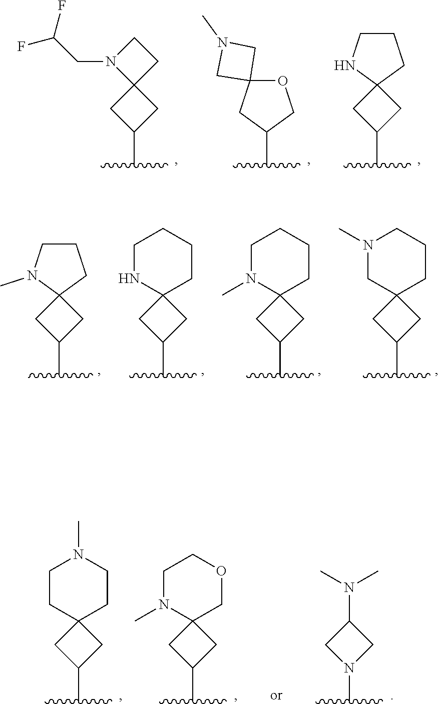

In some embodiments, W is N; Y is CH; Z is O; Ring A is

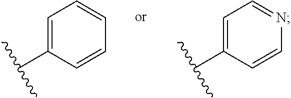 [SMILES: *c1ccccc1 ; "or" ; *c1ccncc1]

X³ is H, methyl, or ethyl; R² is isopropyl; and R¹ is

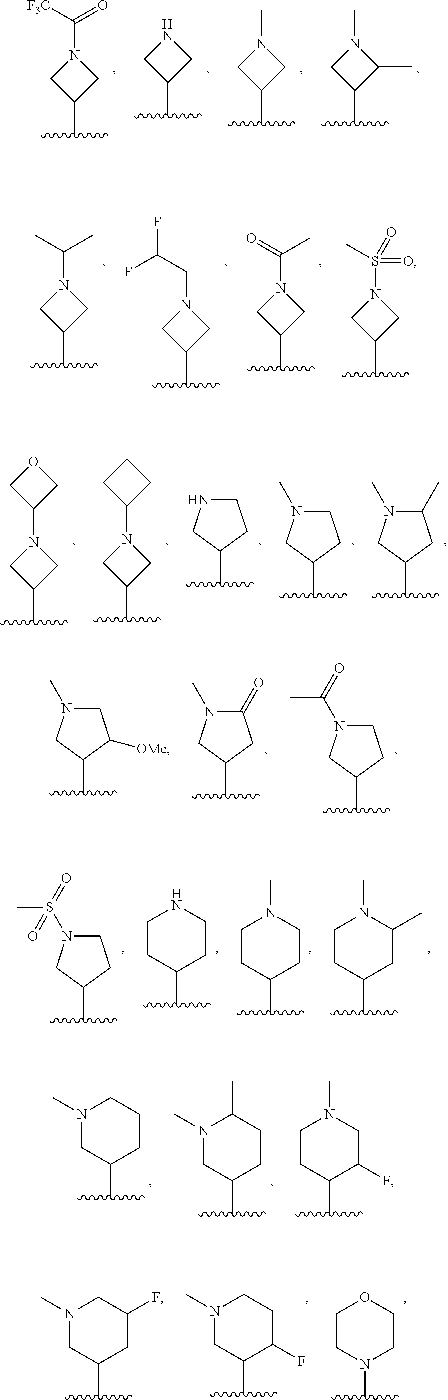

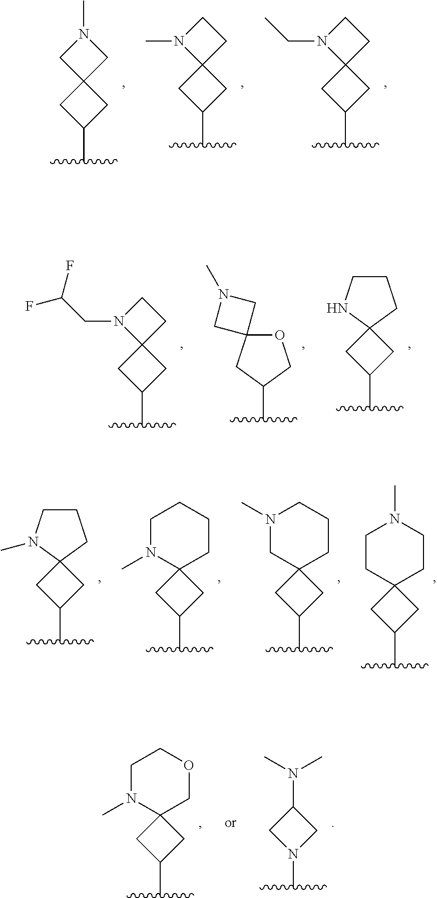

In some embodiments, W is N; Y is CH; Z is O; Ring A is

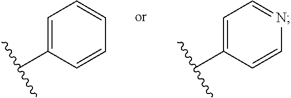 [SMILES: *c1ccccc1 ; "or" ; *c1ccncc1]

X³ is H, methyl, or ethyl; R² is isopropyl; and R¹ is

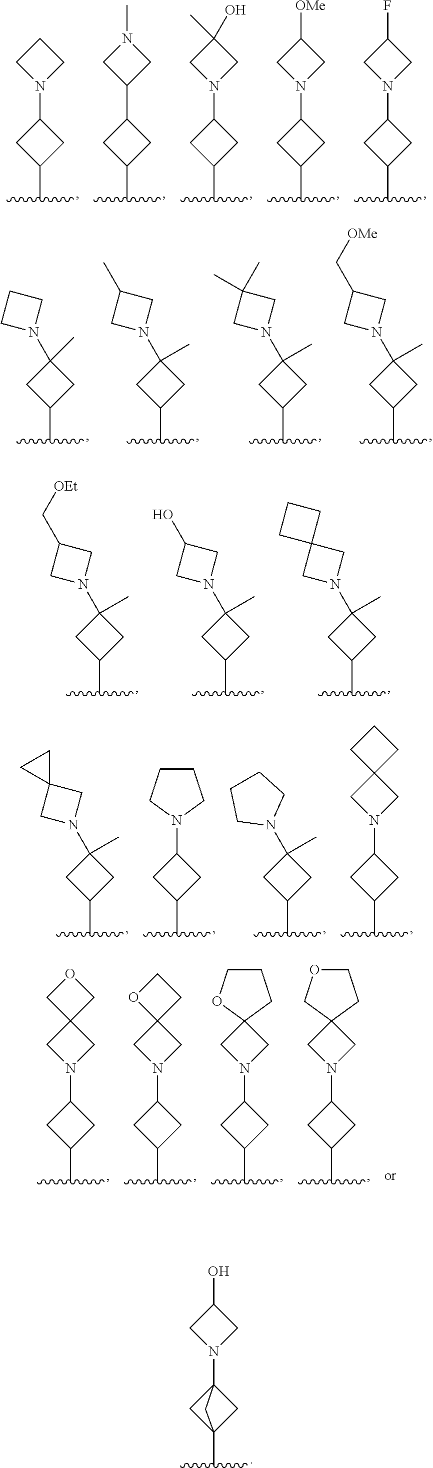

In some embodiments, W is N, Y is CH; Z is O; Ring A is

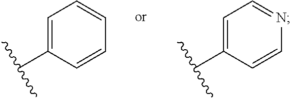 [SMILES: *c1ccccc1 ; "or" ; *c1ccncc1]

R³ is H, methyl, ethyl, isopropyl, tert-butyl,

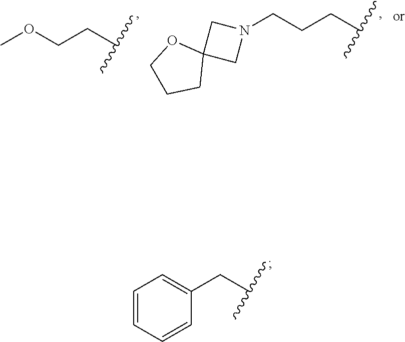

and X³ is H, methyl, or ethyl.

In some embodiments, W is N; Y is CH, Z is O; Ring A is

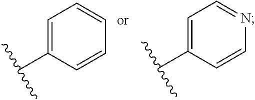 [SMILES: *c1ccccc1 ; *c1ccncc1]

R³ is H, methyl, ethyl, isopropyl, tert-butyl,

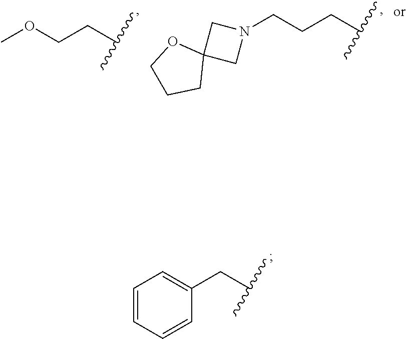

X³ is H, methyl, or ethyl; R² is isopropyl; and R¹ is

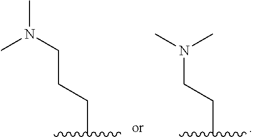

In some embodiments, W is N; Y is CH; Z is O; Ring A is

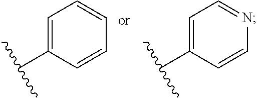 [SMILES: *c1ccccc1 ; "or" ; *c1ccncc1]

R³ is H, methyl, ethyl, isopropyl, tert-butyl,

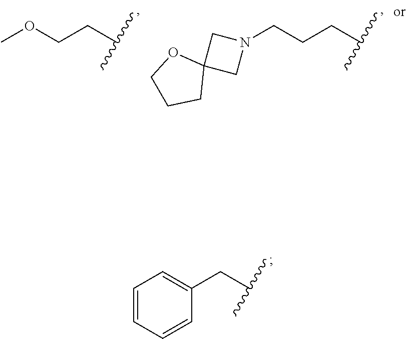

X³ is H, methyl, or ethyl; R² is isopropyl, and R¹ is

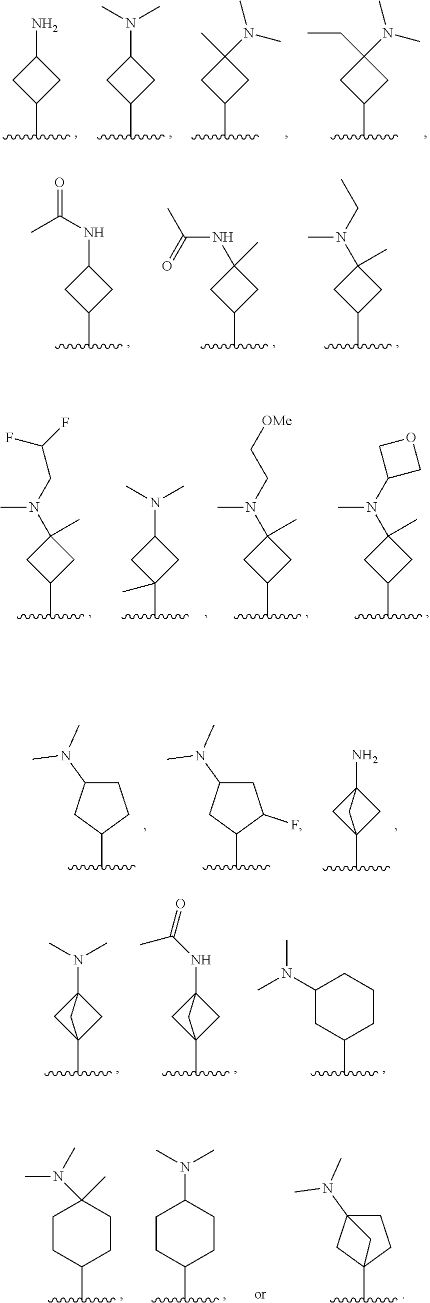

In some embodiments, W is N; Y is CH; Z is O; Ring A is

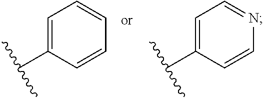 [SMILES: *c1ccccc1 ; *c1ccncc1]

R³ is H, methyl, ethyl, isopropyl, tert-butyl,

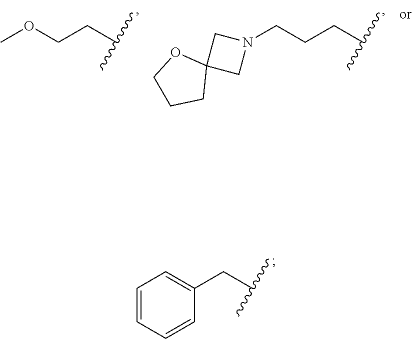

X³ is H, methyl, or ethyl; R² is isopropyl; and R¹ is

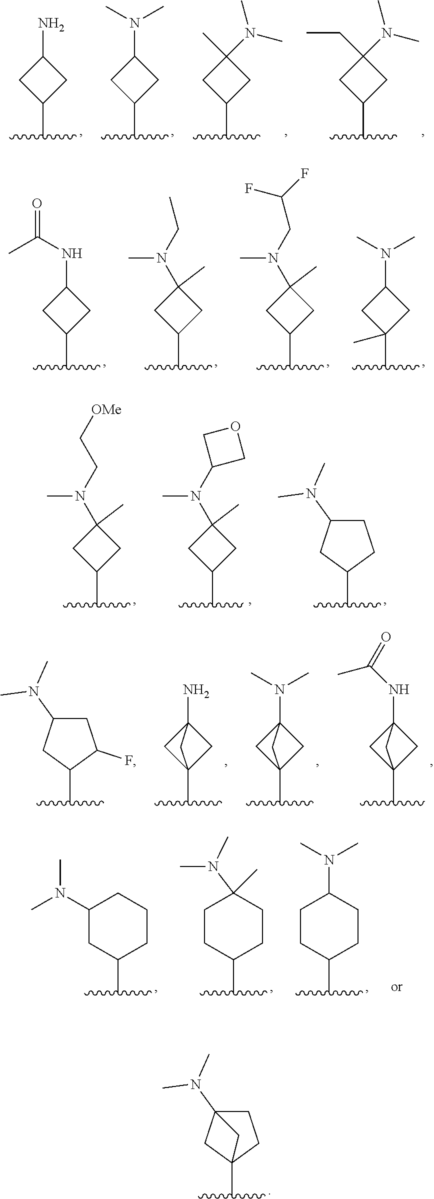

In some embodiments, W is N Y is CH; Z is O; Ring A is

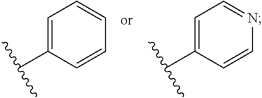 [SMILES: *c1ccccc1 ; *c1ccncc1]

R³ is H, methyl, ethyl, isopropyl, tert-butyl,

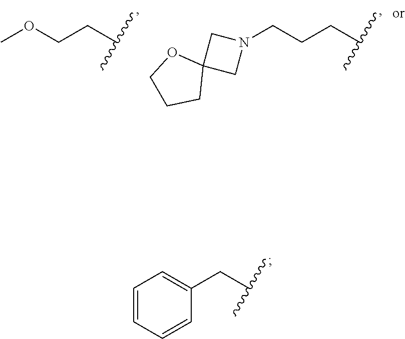

X³ is H, methyl, or ethyl; R² is isopropyl; and R¹ is

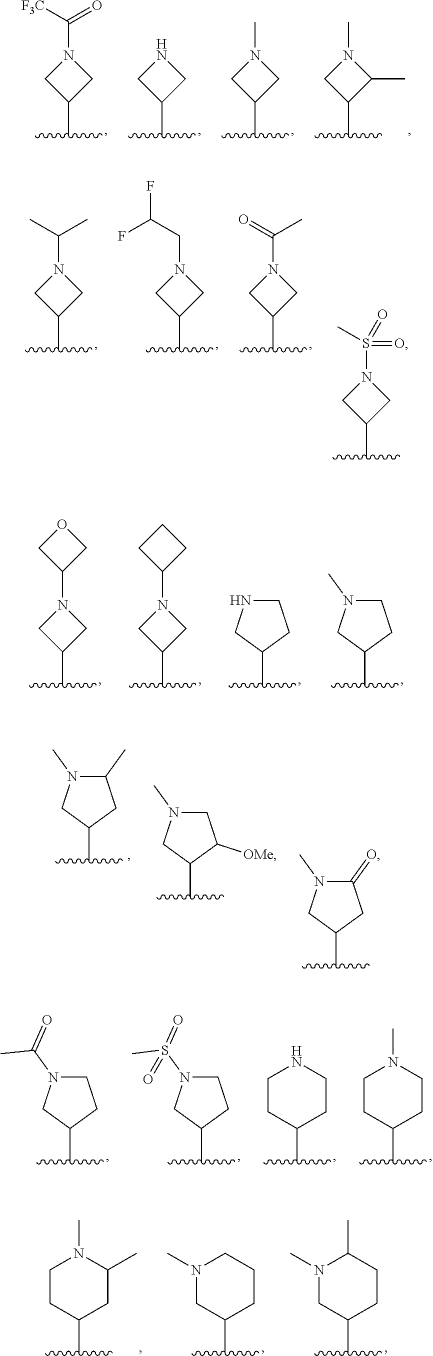

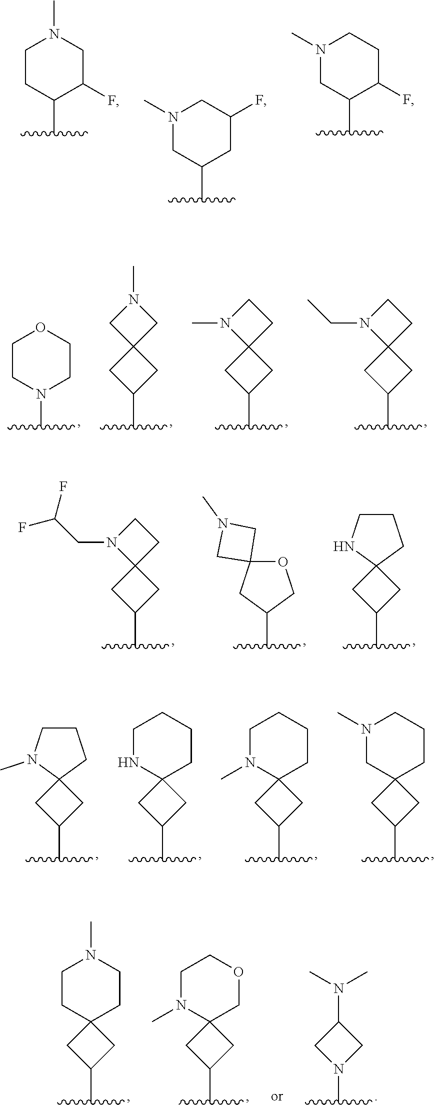

In some embodiments, W is N; Y is CH; Z is O; Ring A is

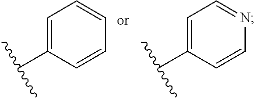 [SMILES: *c1ccccc1 ; "or" ; *c1ccncc1]

R³ is H, methyl, ethyl, isopropyl, tert-butyl,

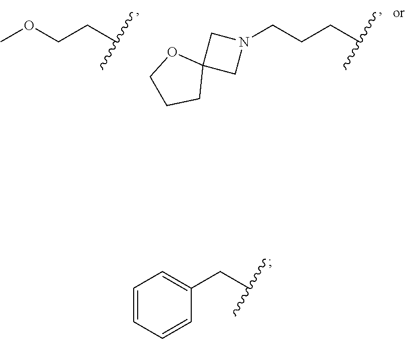

X³ is H, methyl, or ethyl; R² is isopropyl; and R¹ is

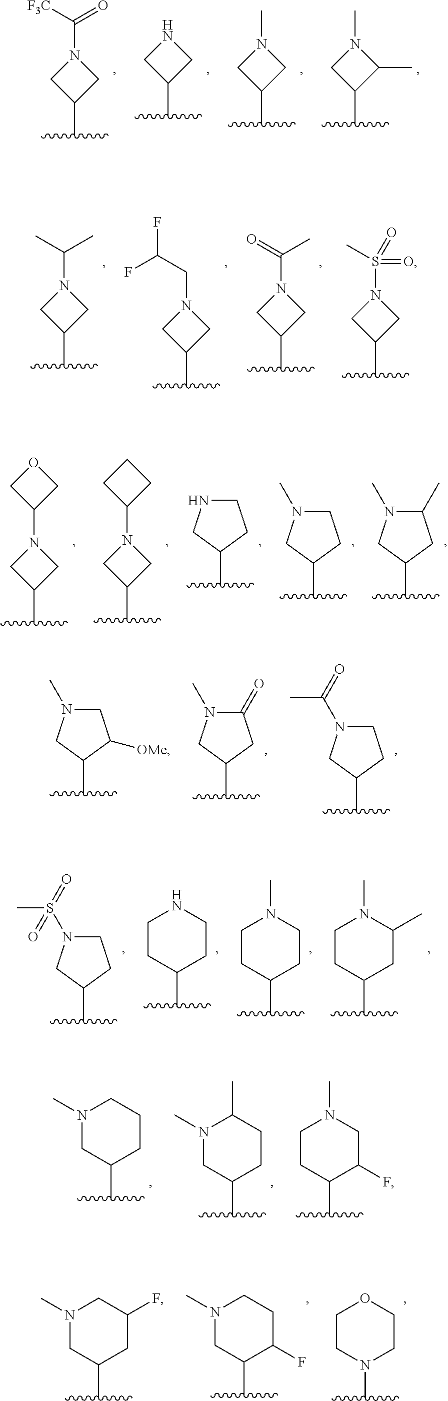

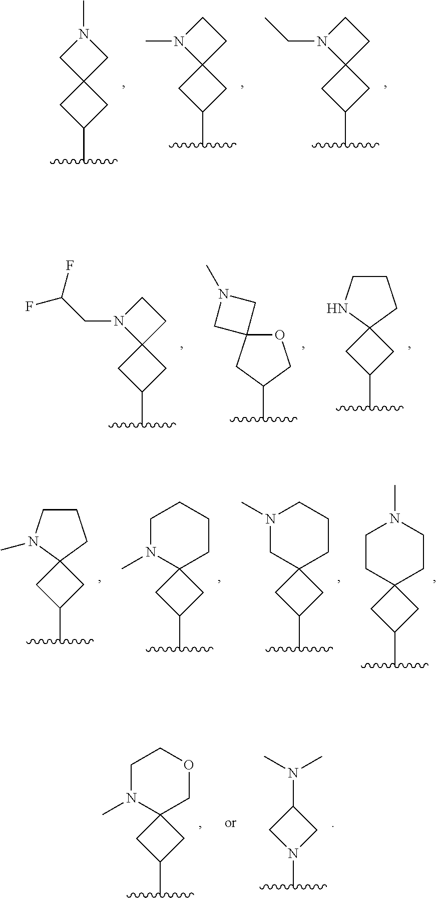

In some embodiments, W is N; Y is CH; Z is O; Ring A is

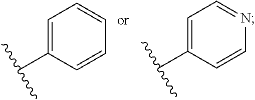 [SMILES: *c1ccccc1 ; *c1ccncc1]

R³ is H, methyl, ethyl, isopropyl, tert-butyl,

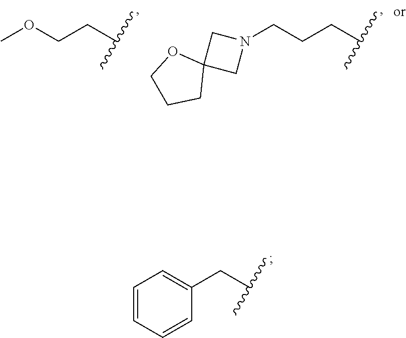

X³ is H, methyl, or ethyl; R² is isopropyl; and R¹ is

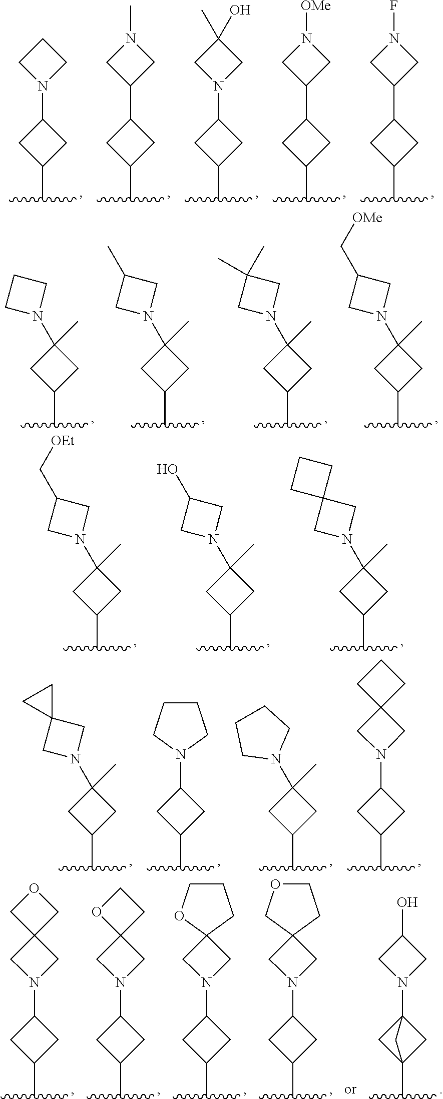

In some embodiments, W is N, Y is CH, Z is O, and

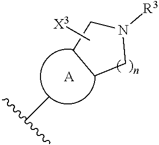

is

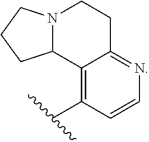 [SMILES: *c1ccnc2c1C1CCCN1CC2]

In some embodiments, W is N; Y is CH, Z is O;

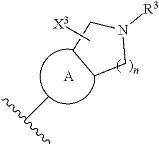

is

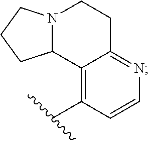 [SMILES: *c1ccnc2c1C1CCCN1CC2]

R¹ is isopropyl; and R¹ is

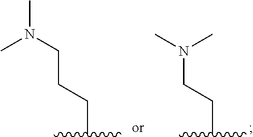

In some embodiments, W is N; Y is CH; Z is O;

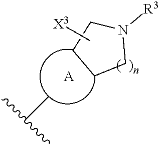

is

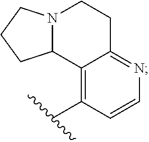 [SMILES: *c1ccnc2c1C1CCCN1CC2]

R² is isopropyl, and R¹ is

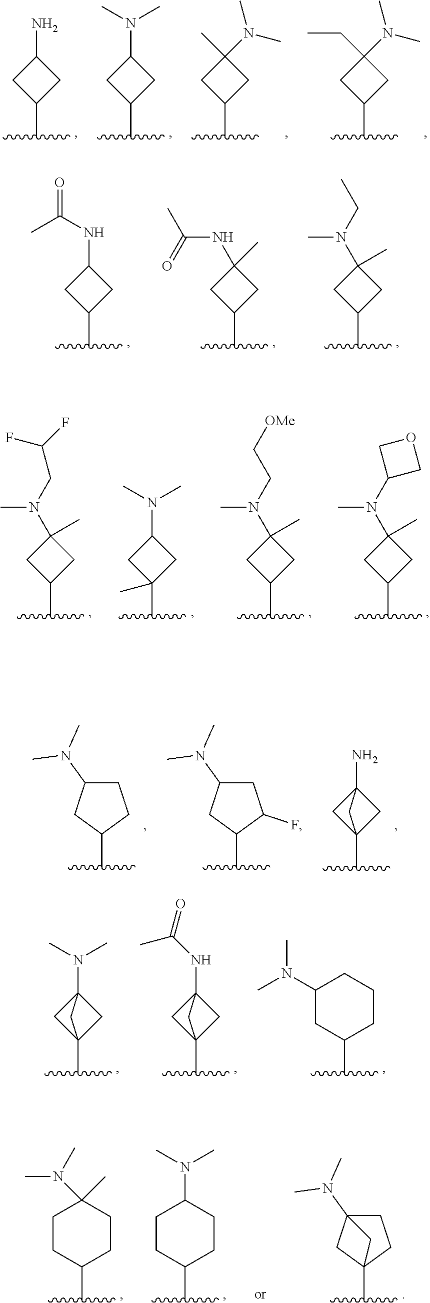

In some embodiments, W is N; Y is CH; Z is O;

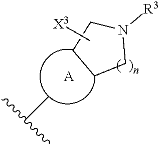

is

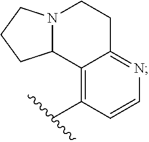 [SMILES: *c1ccnc2c1C1CCCN1CC2]

R² is isopropyl; and R¹ is

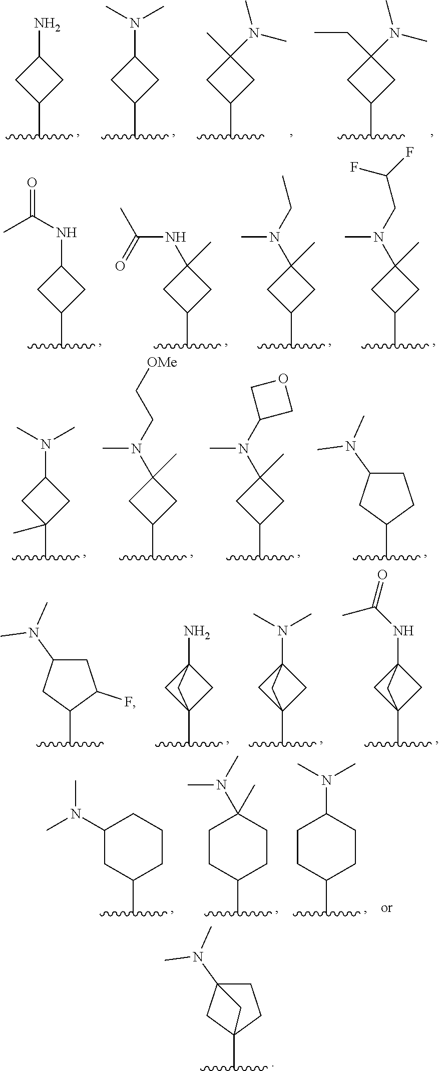

In some embodiments, W is N Y is CH; Z is O;

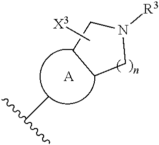

is

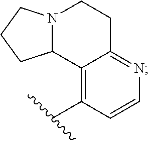 [SMILES: *c1ccnc2c1C1CCCN1CC2]

R² is isopropyl; and R¹ is

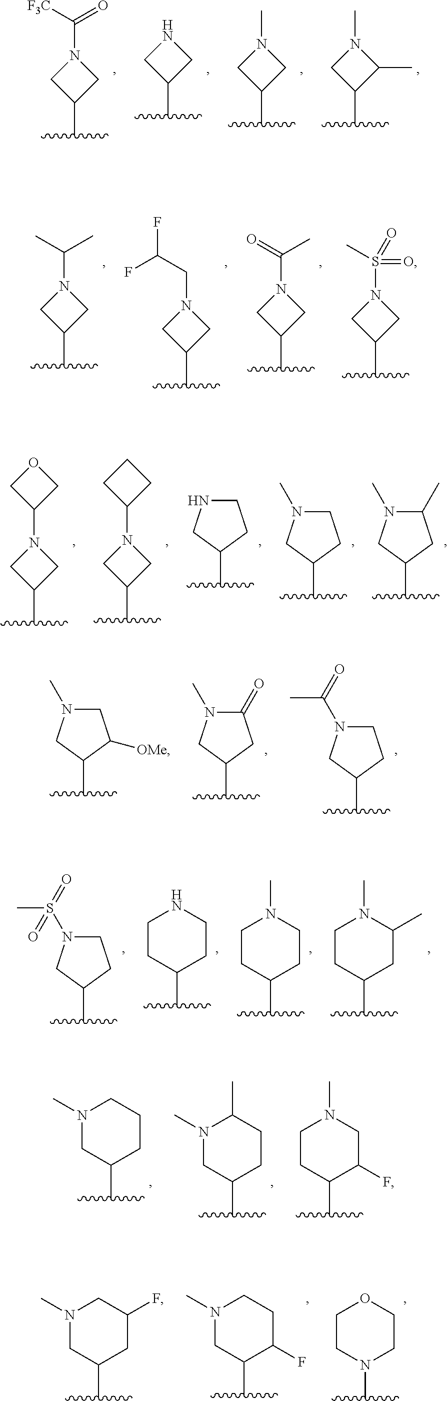

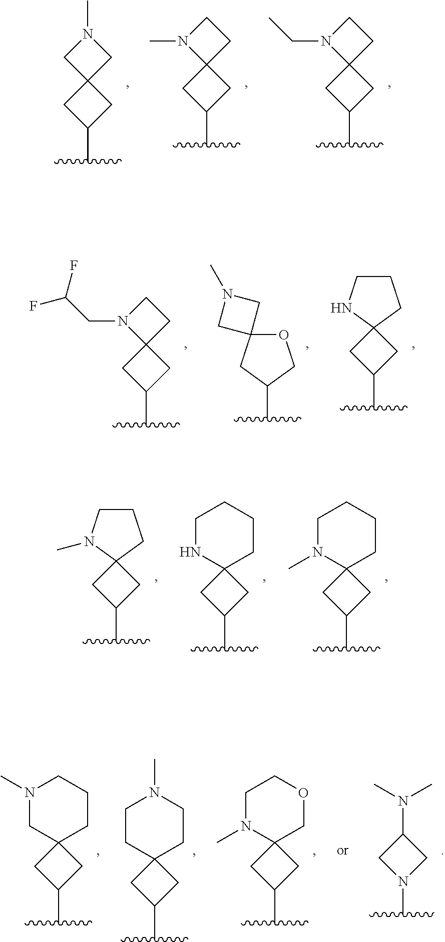

In some embodiments, W is N; Y is CH; Z is O;

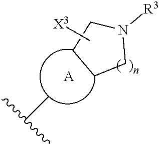

is

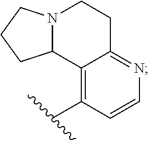 [SMILES: *c1ccnc2c1C1CCCN1CC2]

isopropyl; and R¹ is

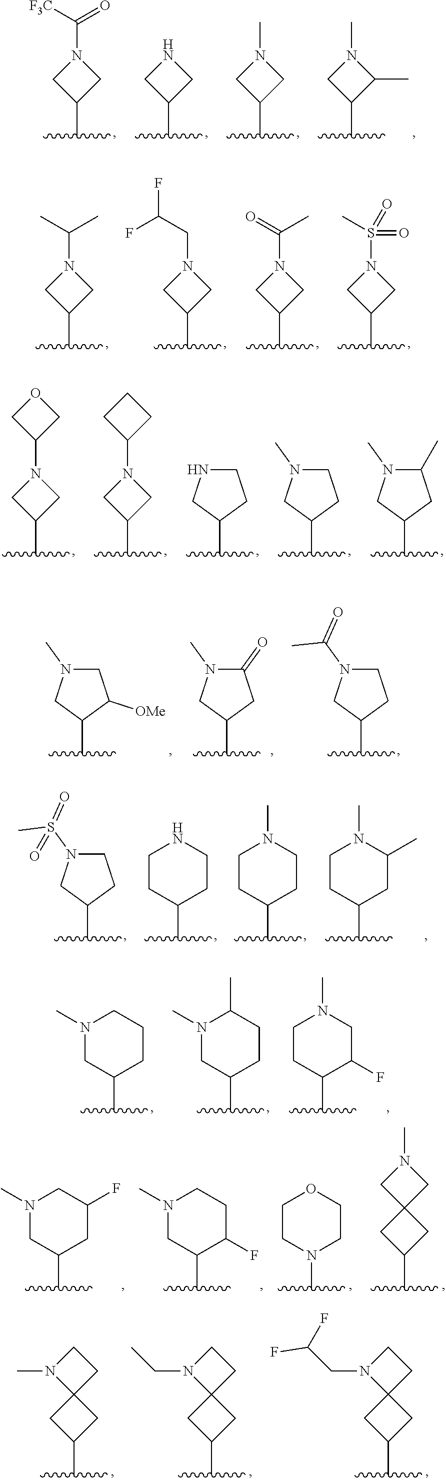

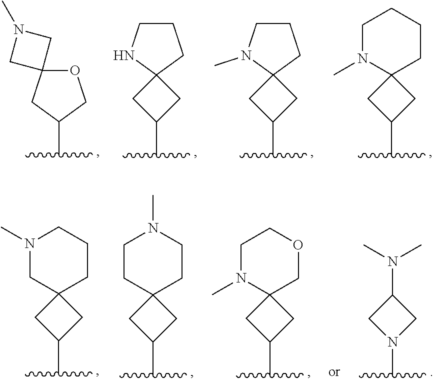

In some embodiments, W is N; Y is CH; Z is O;

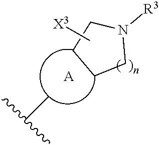

is

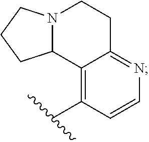 [SMILES: *c1ccnc2c1C1CCCN1CC2]

R² is isopropyl; and R¹ is

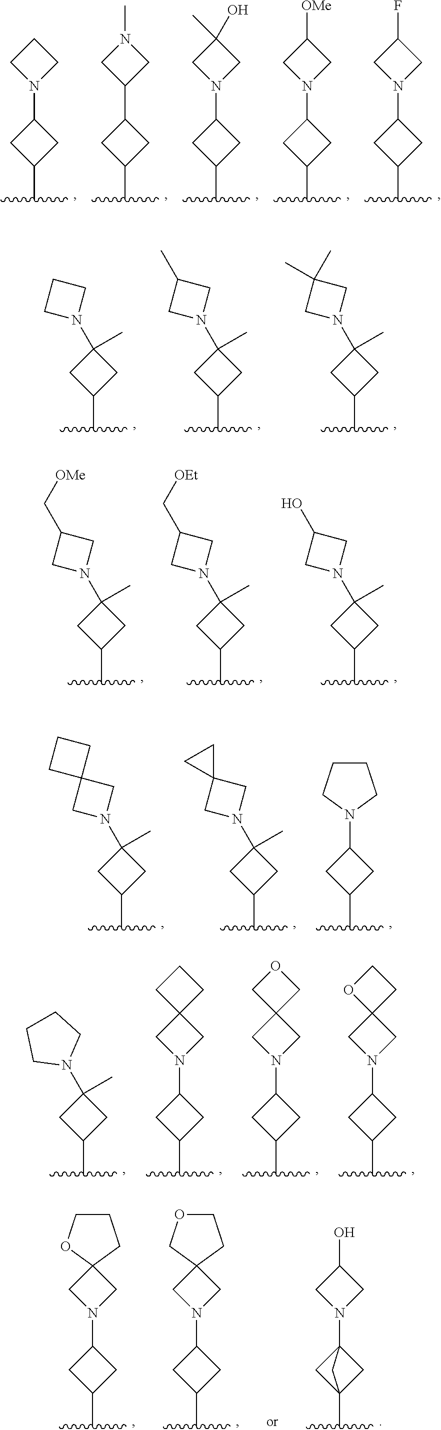

In some embodiments, W is N, Y is CH, Z is O, Ring A is

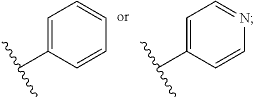 [SMILES: *c1ccccc1 ; "or" ; *c1ccncc1]

and n is 1, 2, or 3.

In some embodiments, W is N; Y is CH; Z is O; Ring A is

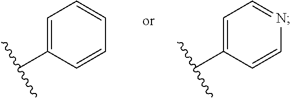 [SMILES: *c1ccccc1 ; *c1ccncc1]

R² is isopropyl; R¹ is

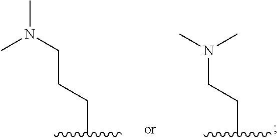

and n is 1, 2, or 3.

In some embodiments, W is N; Y is CH; Z is O; Ring A is

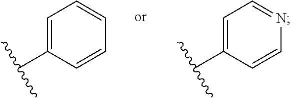 [SMILES: *c1ccccc1 ; "or" ; *c1ccncc1]

R² is isopropyl; R¹ is

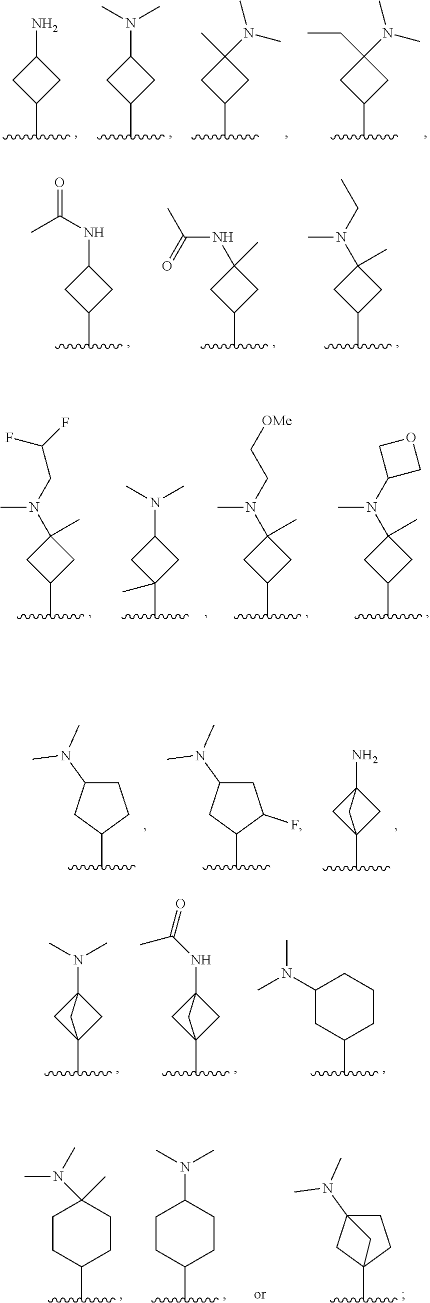

and n is 1, 2, or 3.

In some embodiments, W is N; Y is CH; Z is O; Ring A is

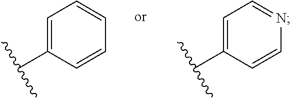 [SMILES: *c1ccccc1 ; "or" ; *c1ccncc1]

R² is isopropyl; R¹ is

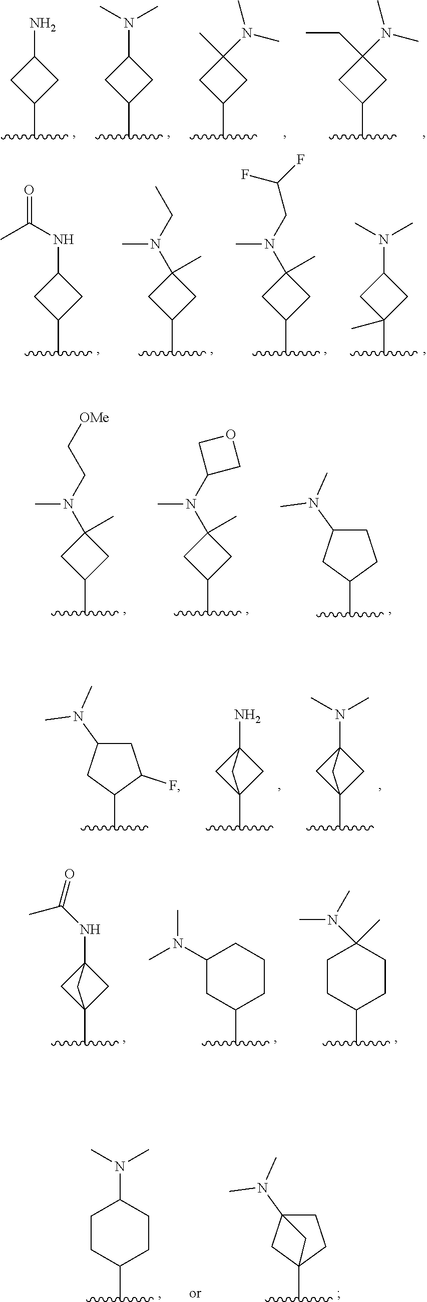

and n is 1, 2, or 3.

In some embodiments, W is N; Y is CH; Z is O; Ring A is

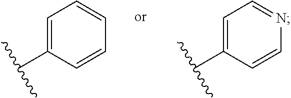 [SMILES: *c1ccccc1 ; "or" ; *c1ccncc1]

R² is isopropyl; R¹ is

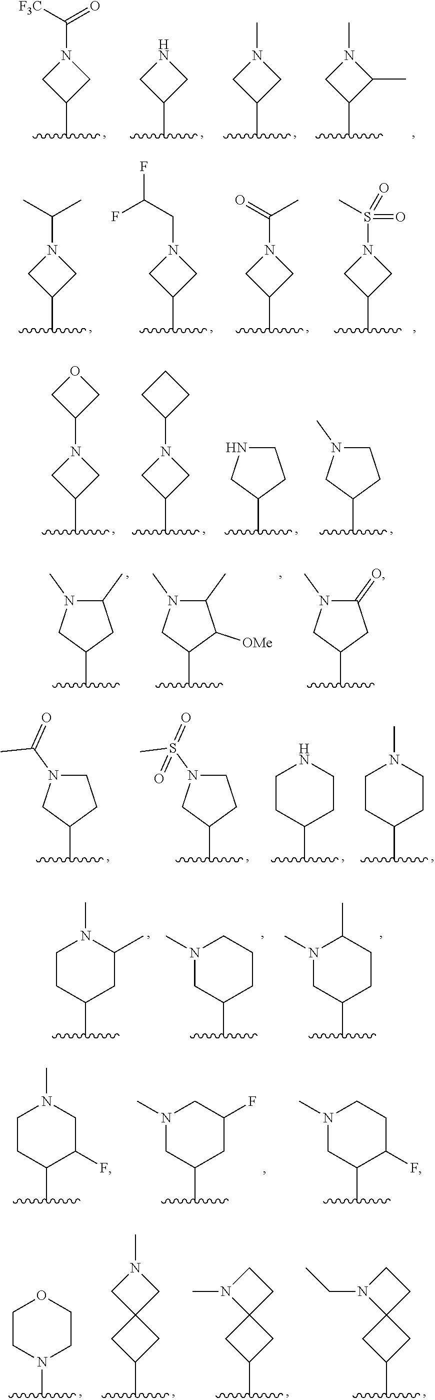

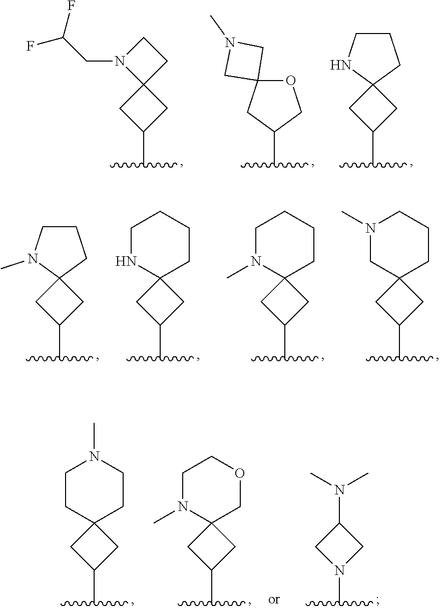

and n is 1, 2, or 3.

In some embodiments W is N; Y is CH; Z is O; Rin A is

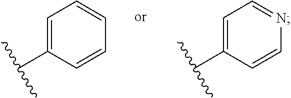 [SMILES: *c1ccccc1 ; "or" ; *c1ccncc1]

R² is isopropyl; R¹ is

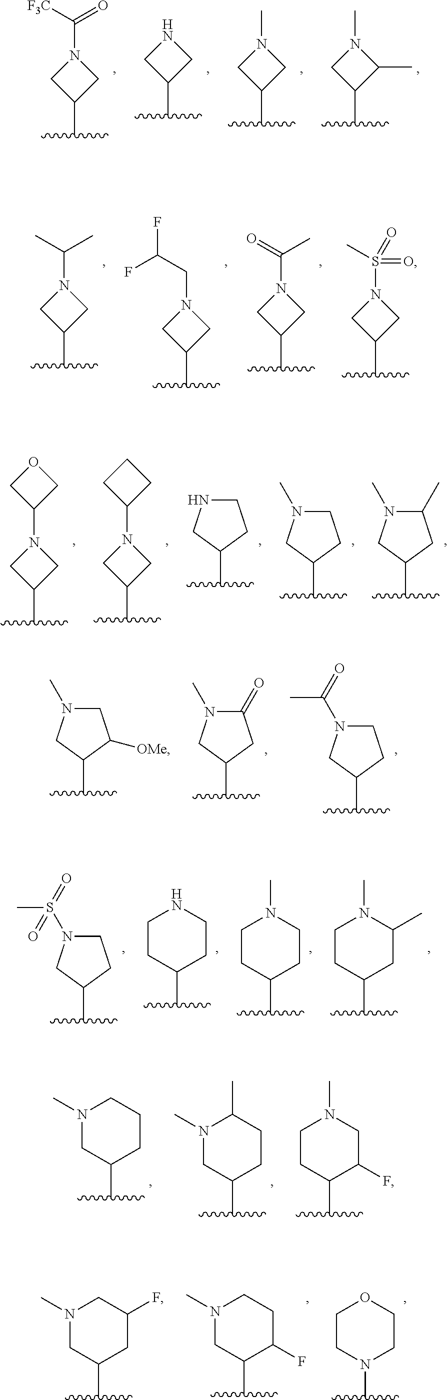

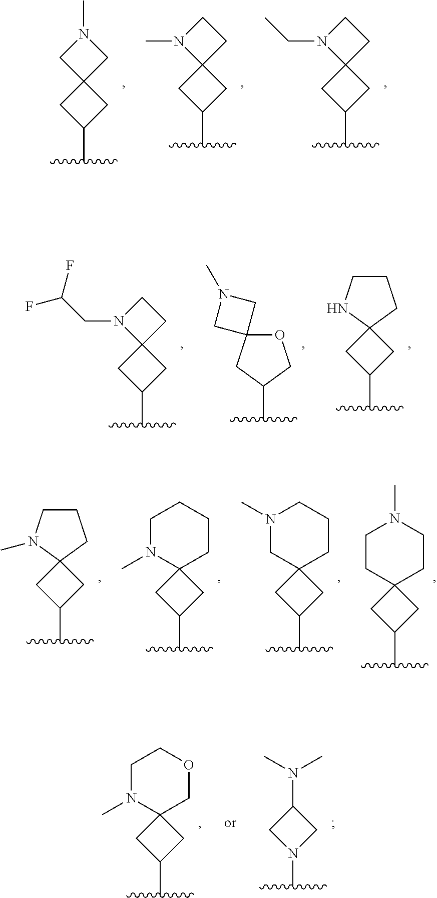

and n is 1, 2, or 3.

In some embodiments, W is N; Y is CH; Z is O; Ring A is

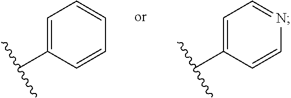 [SMILES: *c1ccccc1 ; "or" ; *c1ccncc1]

R² is isopropyl; R¹ is

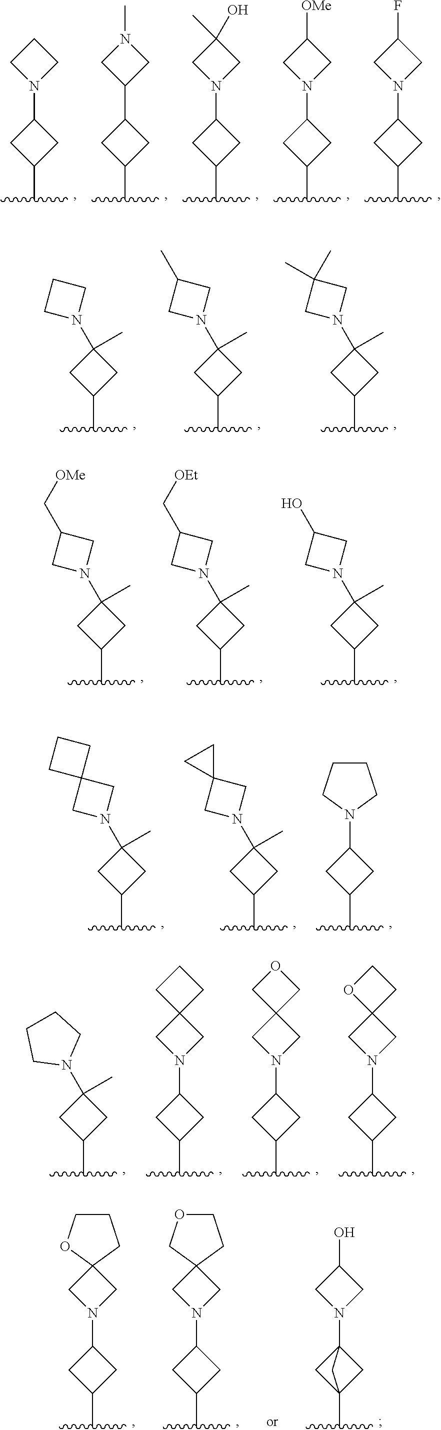

and n is 1, 2, or 3.

In some embodiments, W is N; Y is CH; Z is O; Ring A is

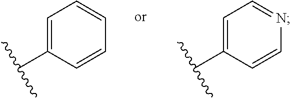 [SMILES: *c1ccccc1 ; "or" ; *c1ccncc1]

X³ is H, methyl, or ethyl; and n is 1, 2, or 3.

In some embodiments, W is N; Y is CH; Z is O; Ring A is

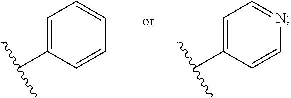 [SMILES: *c1ccccc1 ; *c1ccncc1]

X³ is H, methyl, or ethyl; R² is isopropyl; R¹ is

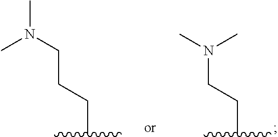

and n is 1, 2, or 3.

In some embodiments, W is N; Y is CH; Z is O; Ring A is

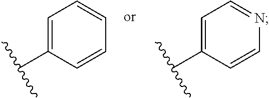 [SMILES: *c1ccccc1 ; "or" ; *c1ccncc1]

X³ is H, methyl, or ethyl; R² is isopropyl; R¹ is

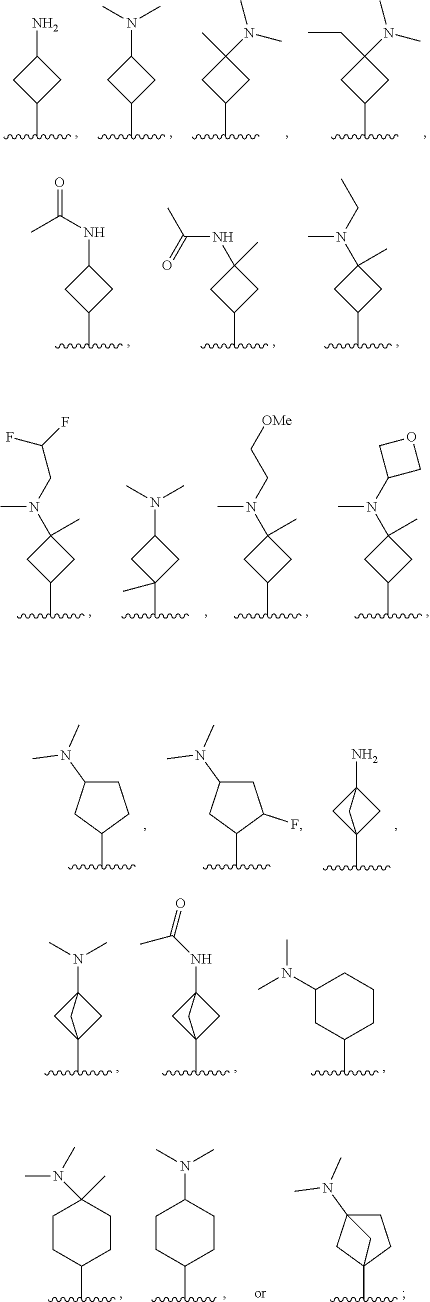

and n is 1, 2, or 3.

In some embodiments, W is N; Y is CH; Z is O; Ring A is

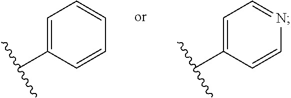 [SMILES: *c1ccccc1 ; "or" ; *c1ccncc1]

X³ is H, methyl, or ethyl; R² is isopropyl; R¹ is

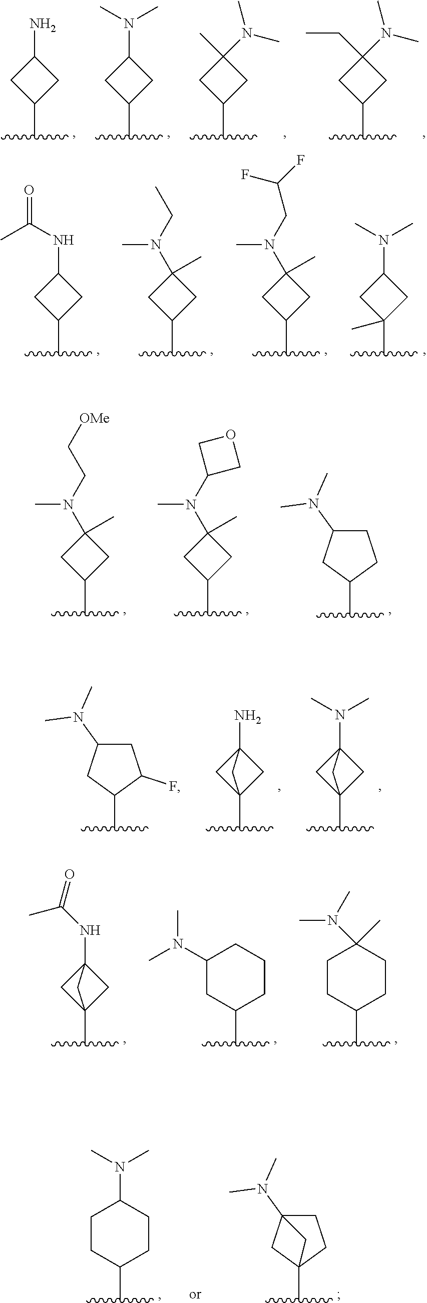

and n is 1, 2, or 3.

In some embodiments, W is N; Y is CH; Z is O; Ring A is

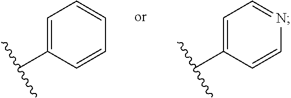 [SMILES: *c1ccccc1 ; "or" ; *c1ccncc1]

X³ is H, methyl, or ethyl; R² is isopropyl; R¹ is

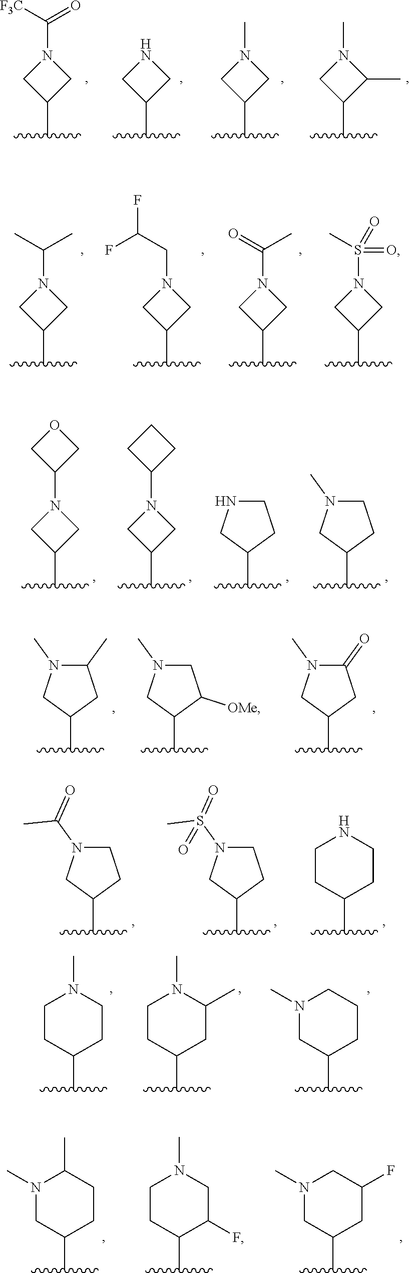

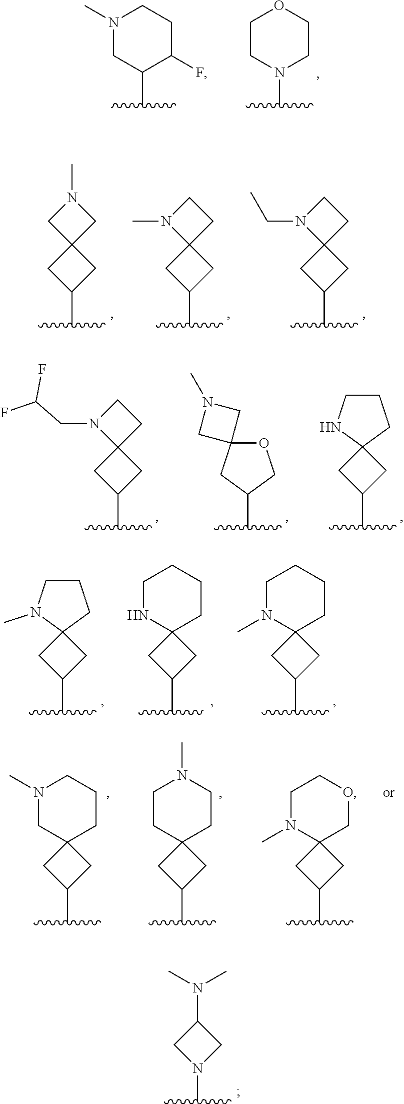

and n is 1, 2, or 3.

In some embodiments, W is N; Y is CH; Z is O; Ring A is

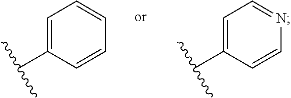 [SMILES: *c1ccccc1 ; "or" ; *c1ccncc1]

X³ is H, methyl, or ethyl; R² is isopropyl; R¹ is

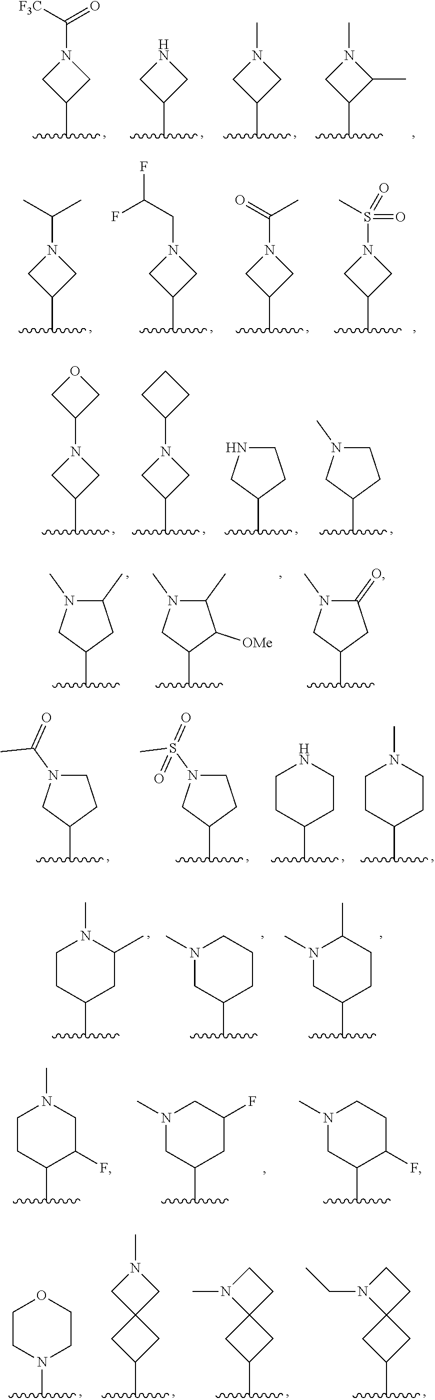

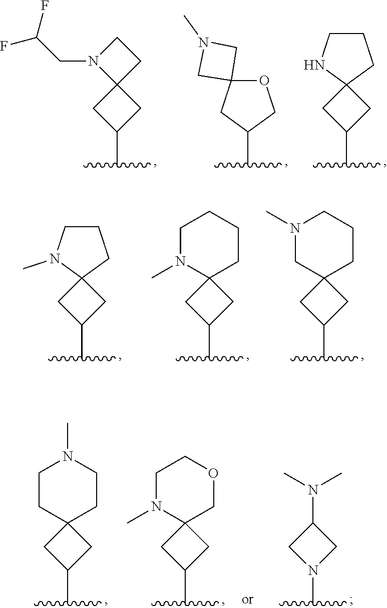

and n is 1, 2, or 3.1

In some embodiments, W is N; Y is CH; Z is O; Ring A is

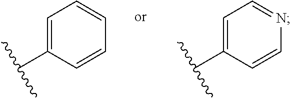 [SMILES: *c1ccccc1 ; "or" ; *c1ccncc1]

X³ is H, methyl, or ethyl; R² is isopropyl; R¹ is

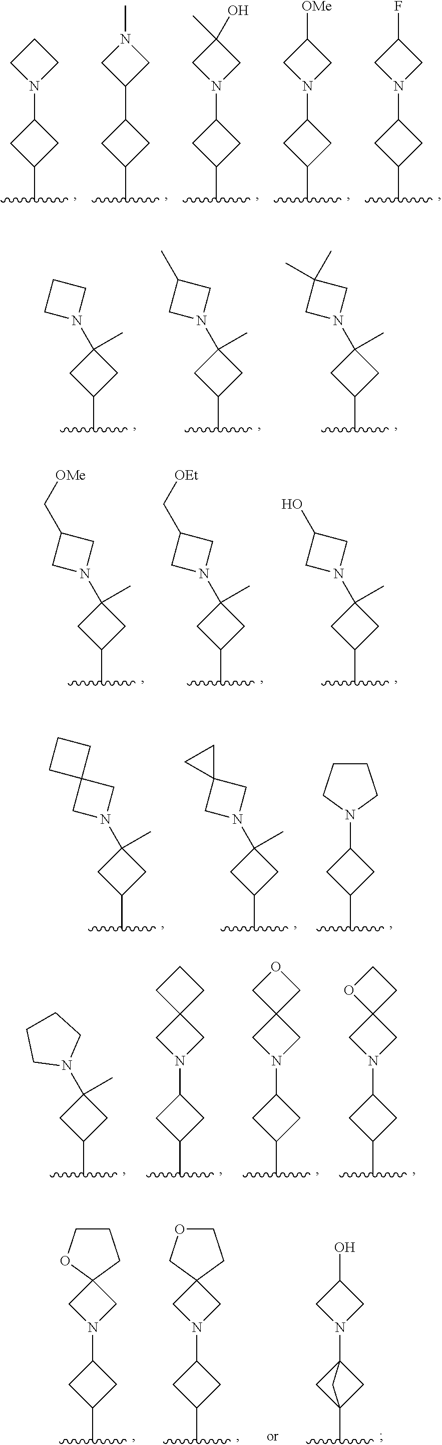

n is 1, 2, or 3.

In some embodiments, W is N; Y is CH; Z is O; Ring A is

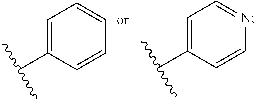 [SMILES: *c1ccccc1 ; *c1ccncc1]

R³ is H, methyl, ethyl, isopropyl, tert-butyl,

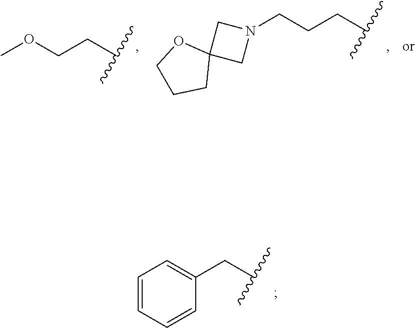

X³ is H, methyl, or ethyl; and n is 1, 2, or 3.

In some embodiments, W is N; Y is CH; Z is O; Ring A is

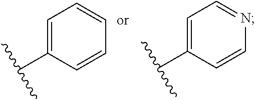 [SMILES: *c1ccccc1 ; *c1ccncc1]

R³ is H, methyl, ethyl, isopropyl, tert-butyl,

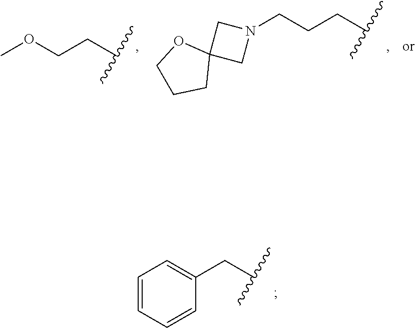

X³ is H, methyl, or ethyl; R² is isopropyl; R¹ is

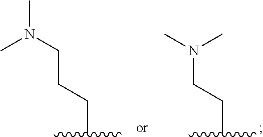

and n is 1, 2, or 3.

In some embodiments, W is N; Y is CH; Z is O; Ring A is

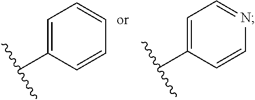 [SMILES: *c1ccccc1 ; *c1ccncc1]

R³ is H, methyl, ethyl, isopropyl, tert-butyl,

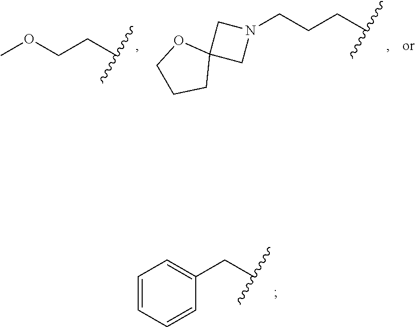

X³ is H, methyl, or ethyl; R² is isopropyl; R¹ is

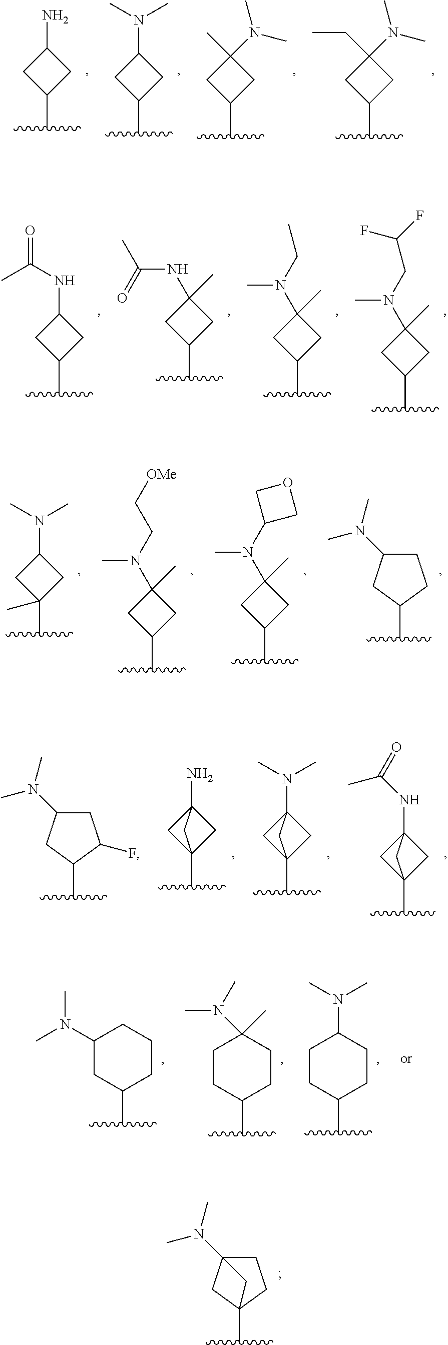

and n is 1, 2, or 3.

In some embodiments, W is N; Y is CH; Z is O; Ring A is

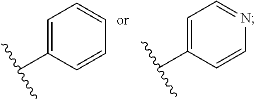 [SMILES: *c1ccccc1 ; *c1ccncc1]

R³ is H, methyl, ethyl, isopropyl, tert-butyl,

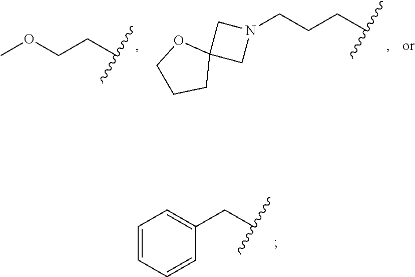

R³ is H, methyl, or ethyl; R² is isopropyl; R¹ is

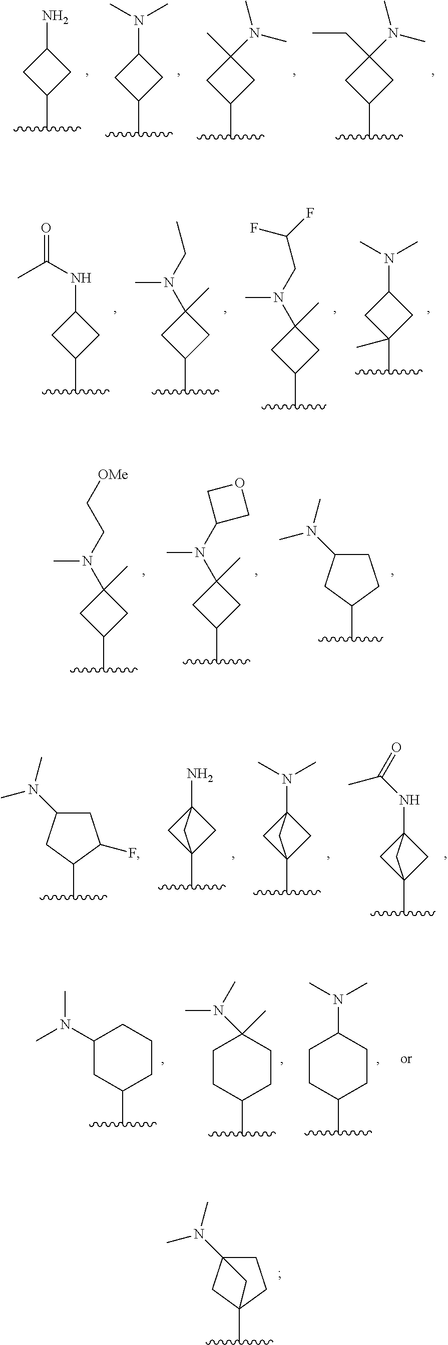

and n is 1, 2, or 3.

In some embodiments, W is N; Y is CH; Z is O; Ring A is

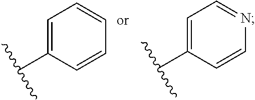 [SMILES: *c1ccccc1 ; *c1ccncc1]

R³ is H, methyl, ethyl, isopropyl, tert-butyl,

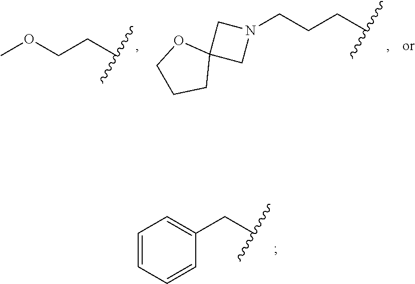

X³ is H, methyl, or ethyl; R² is isopropyl; R¹ is

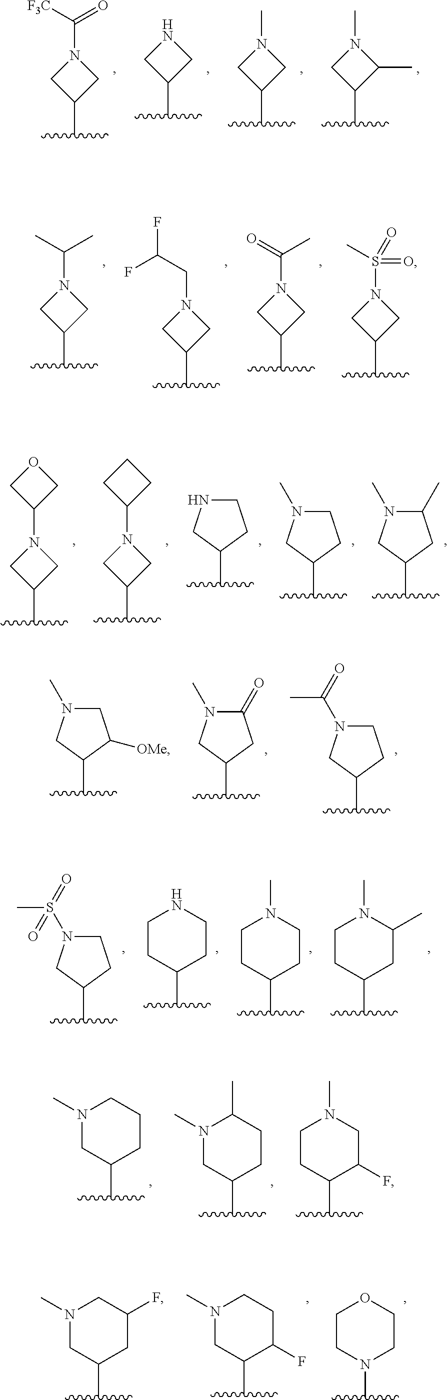

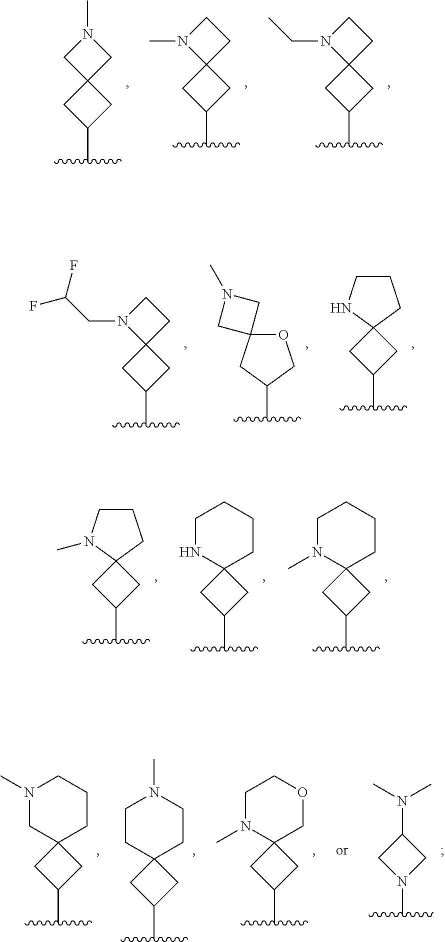

In some embodiments, W is N; Y is CH; Z is O; Ring A is

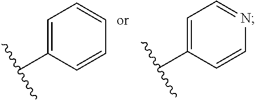 [SMILES: *c1ccccc1 ; *c1ccncc1]

R³ is H, methyl, ethyl, isopropyl, tert-butyl,

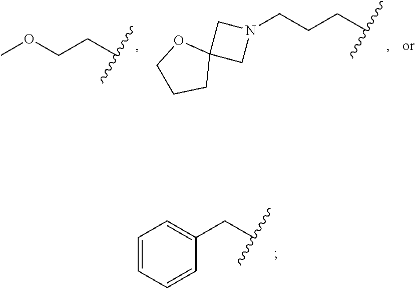

X³ is H, methyl, or ethyl; R² is isopropyl; R¹ is

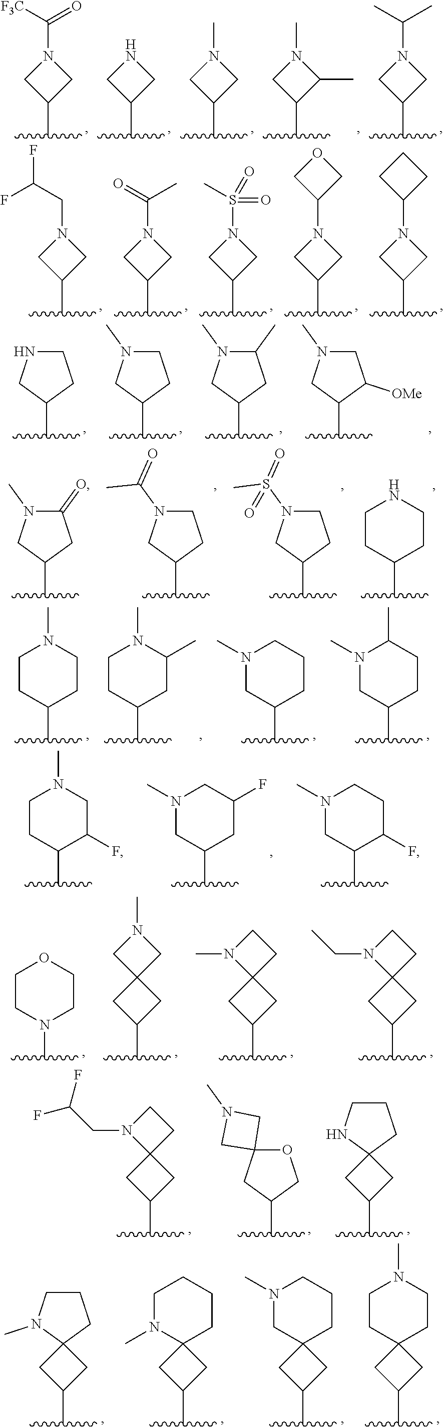

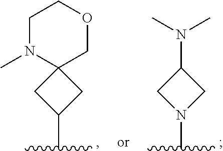

and n is 1, 2, or 3.

In some embodiments, W is N; Y is CH; Z is O; Ring A is

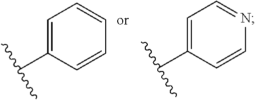 [SMILES: *c1ccccc1 ; *c1ccncc1]

R³ is H, methyl, ethyl, isopropyl, tert-butyl,

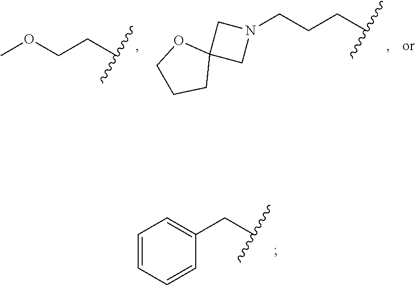

X³ is H, methyl, or ethyl; R² is isopropyl; R¹ is

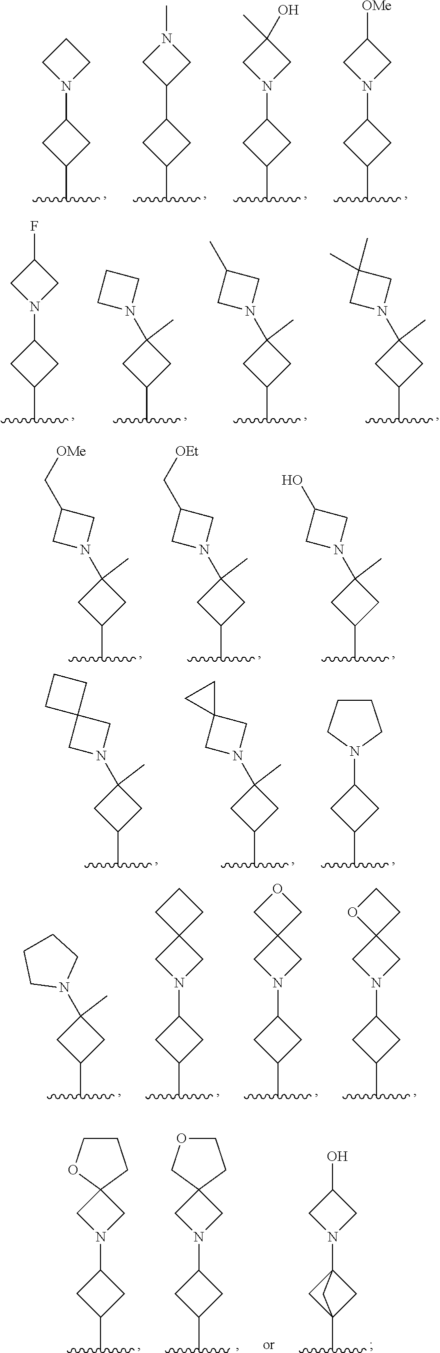

and n is, 2, or 3.

In some embodiments, W is N; Y is N; Z is O; Ring A is

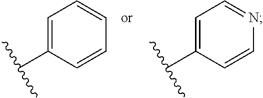 [SMILES: *c1ccccc1 ; *c1ccncc1]

### R³ is

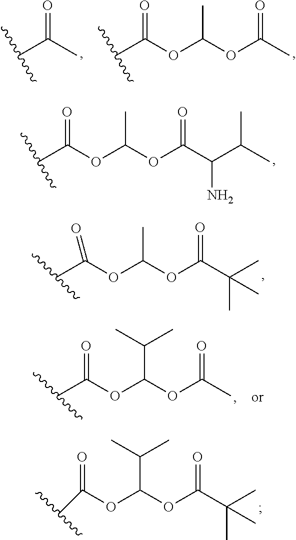

and X is H, methyl, or ethyl.

In some embodiments, W is N; Y is N; Z is O; Ring A is

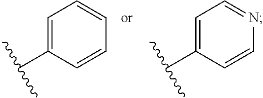 [SMILES: *c1ccccc1 ; *c1ccncc1]

### R³ is

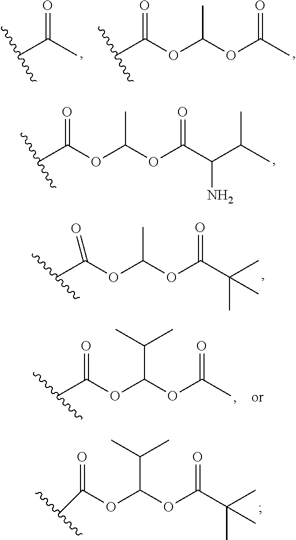

X³ is H, methyl, or ethyl; R² is isopropyl; and R¹ is

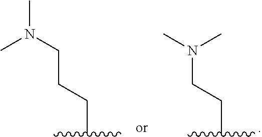

In some embodiments, W is N; Y is N; Z is O; Ring A is

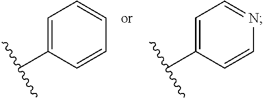 [SMILES: *c1ccccc1 ; *c1ccncc1]

### R³ is

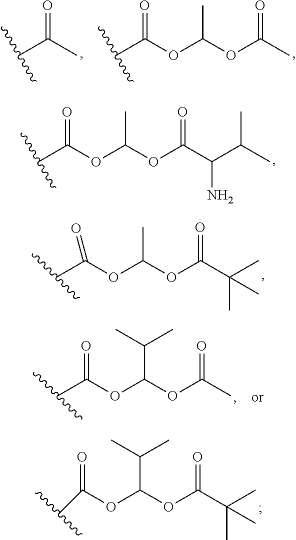

X³ is H, methyl, or ethyl; R² is isopropyl; and R¹ is

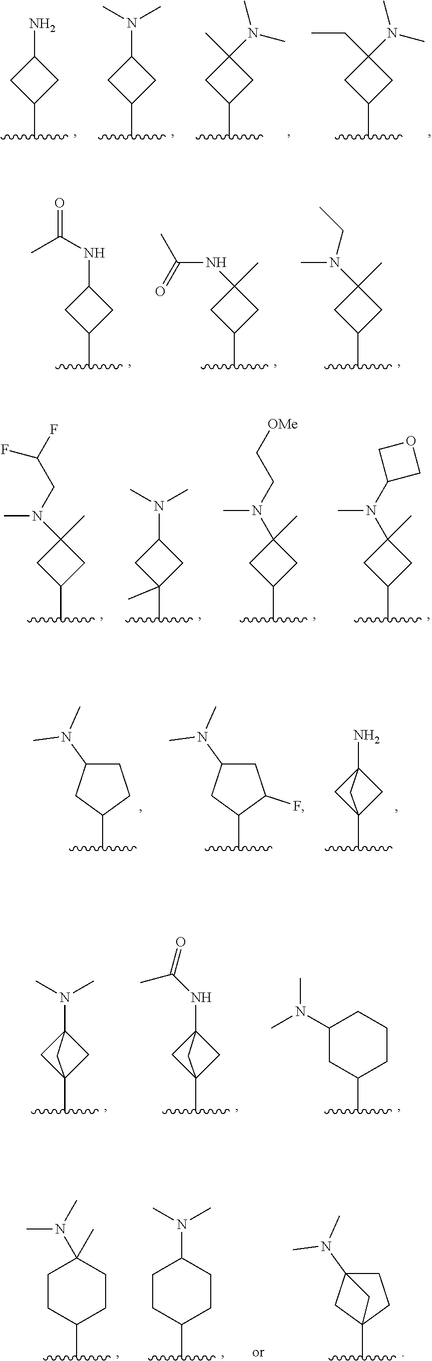

In some embodiments, W is N; Y is N; Z is O; Ring A is

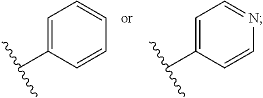 [SMILES: *c1ccccc1 ; *c1ccncc1]

### R³ is

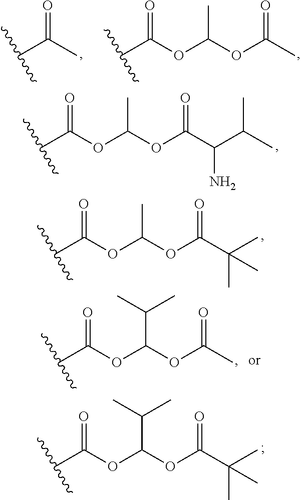

X³ is H, methyl, or ethyl; R² is isopropyl; and R¹ is

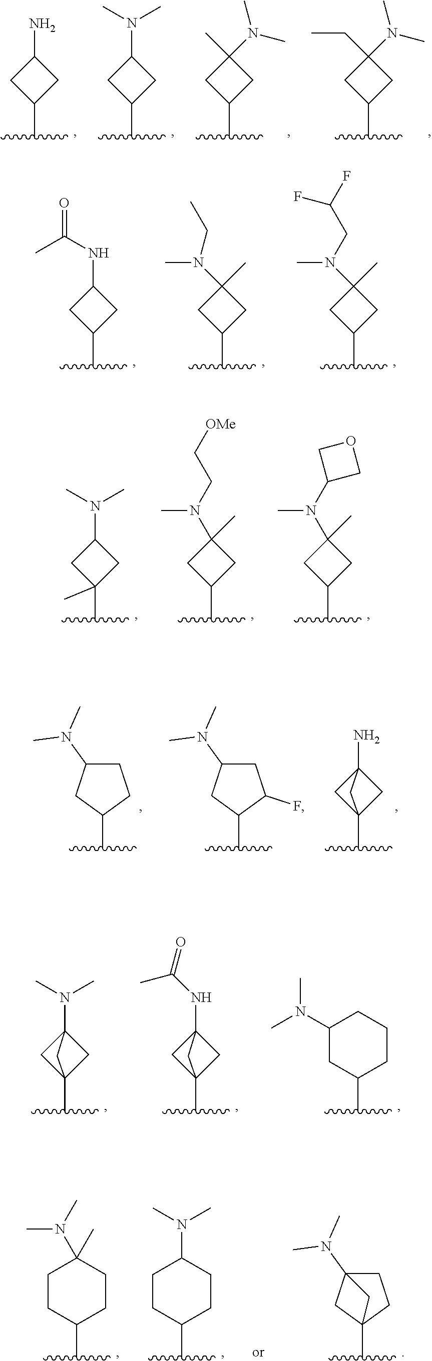

In some embodiments, W is N; Y is N, Z is O; Ring A is

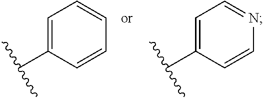 [SMILES: *c1ccccc1 ; *c1ccncc1]

### R³

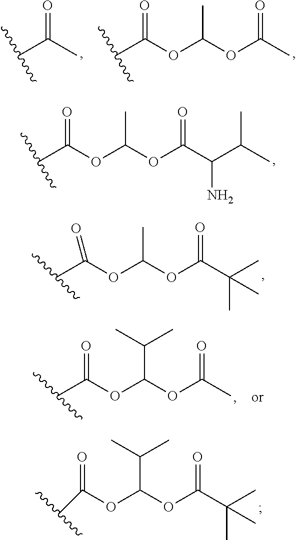

X³ is H, methyl, or ethyl; R² is isopropyl; and R¹ is

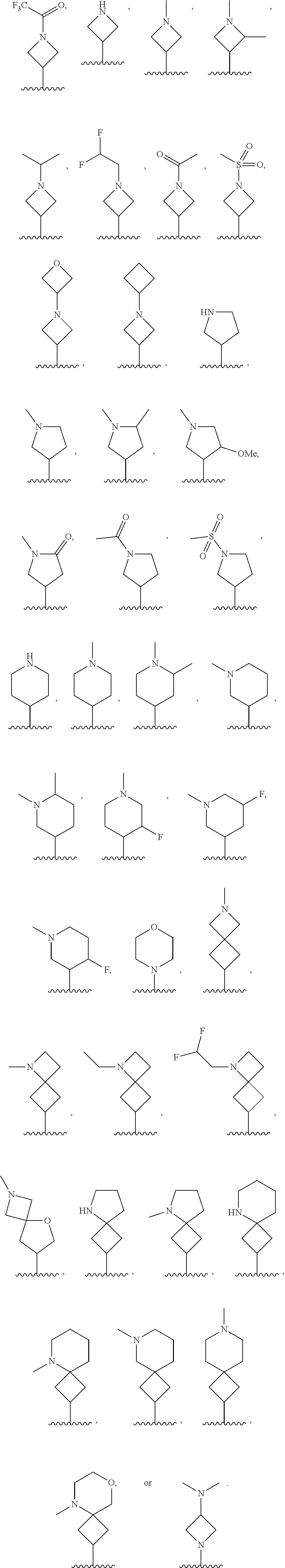

In some embodiments, W is N; Y is N; Z is O; Ring A is

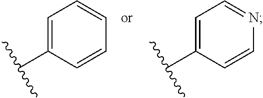 [SMILES: *c1ccccc1 ; *c1ccncc1]

### R³

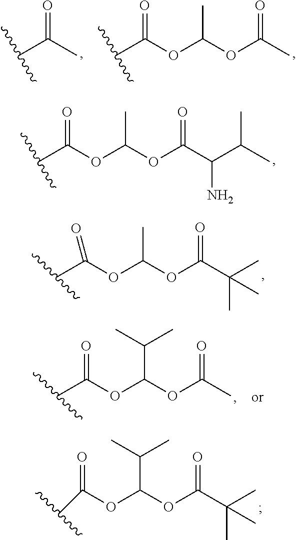

X³ is H, methyl, or ethyl; R² is isopropyl; and R¹ is

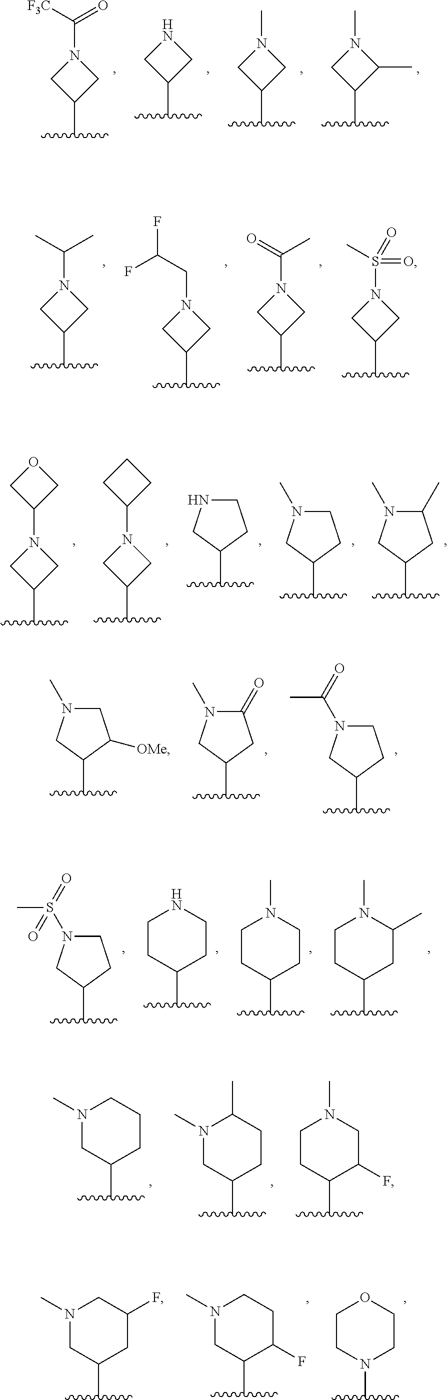

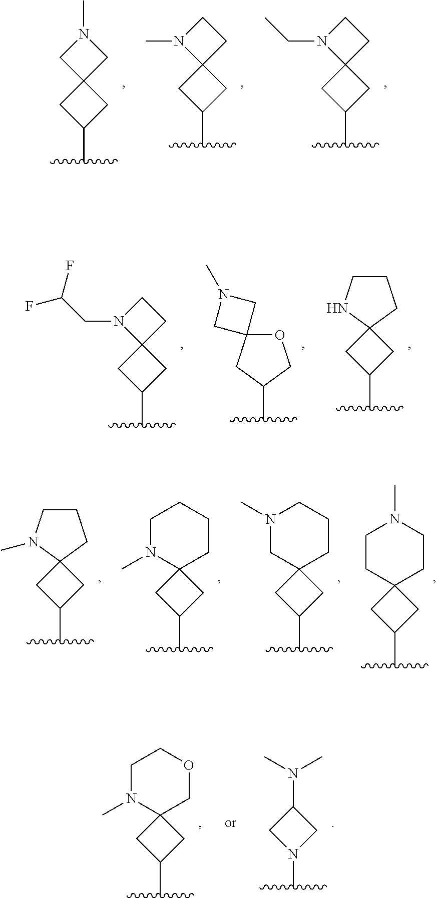

In some embodiments, W is N; Y is N; Z is O; Ring A is

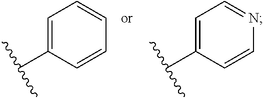 [SMILES: *c1ccccc1 ; *c1ccncc1]

### R³ is

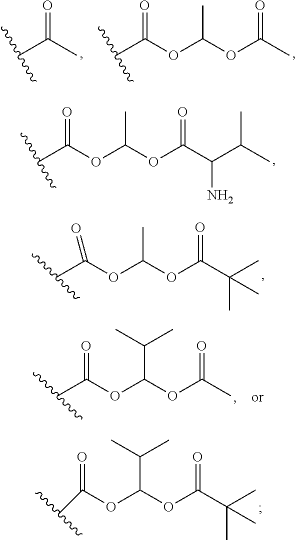

X³ is H, methyl, or ethyl; R² is isopropyl; and R¹ is

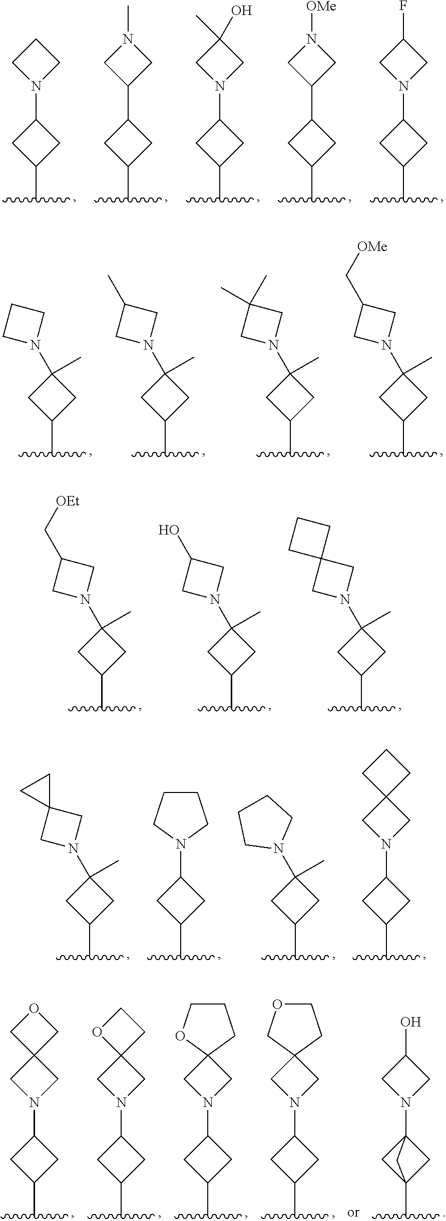

In some embodiments, W is N; Y is N; Z is O; Ring A is

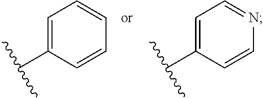 [SMILES: *c1ccccc1 ; *c1ccncc1]

### R³ is

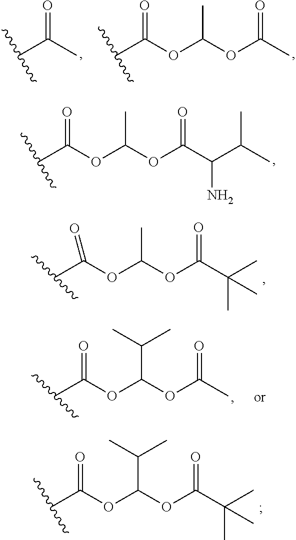

X³ is H, methyl, or ethyl; and n is 1, 2, or 3.

In some embodiments, W is N; Y is N; Z is O; Ring A is

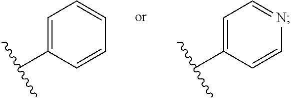 [SMILES: *c1ccccc1 ; "or" ; *c1ccncc1]

### R³ is

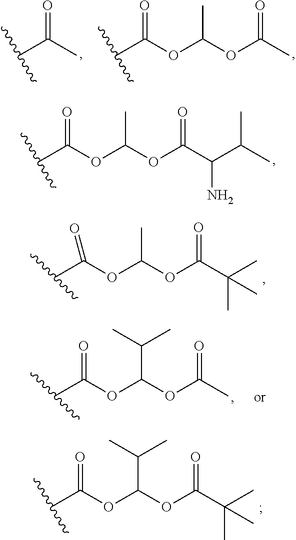

X³ is H, methyl, or ethyl; R² is isopropyl, R¹ is

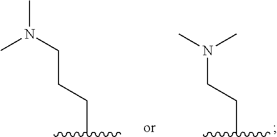

and n is 1, 2, or 3.

In some embodiments, W is N; Y is N; Z is O; Ring A is

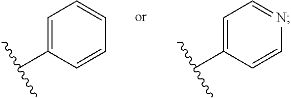 [SMILES: *c1ccccc1 ; "or" ; *c1ccncc1]

### R³ is

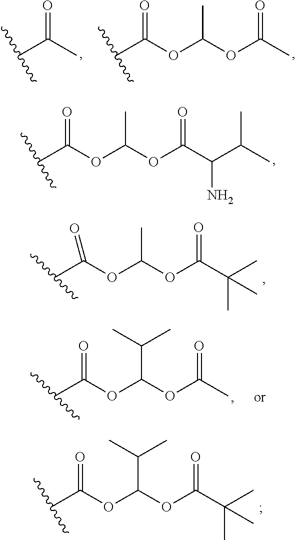

X³ is H, methyl, or ethyl; R² is isopropyl, R¹ is

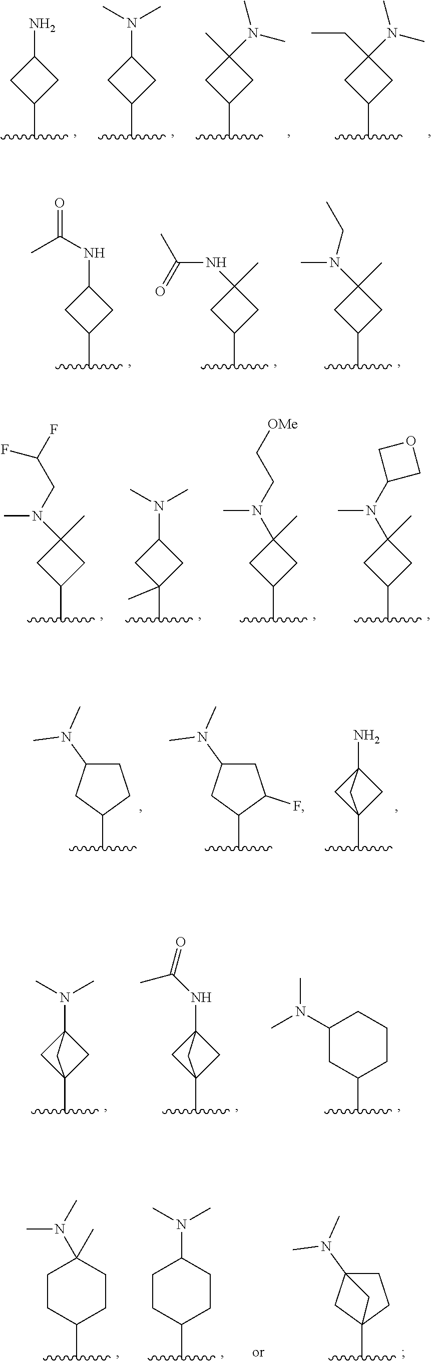

and n is 1, 2, or 3.

In some embodiments, W is N; Y is N; Z is O; Ring A is

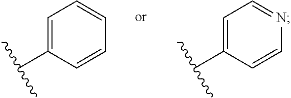 [SMILES: *c1ccccc1 ; "or" ; *c1ccncc1]

### R³ is

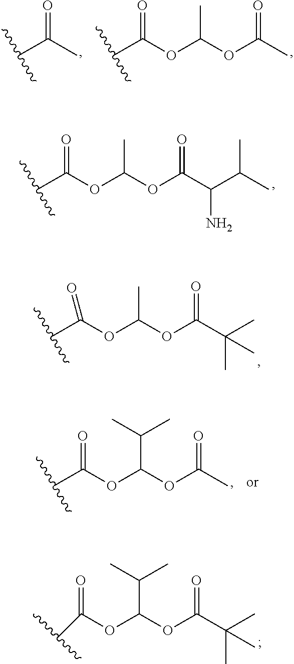

X³ is H, methyl, or ethyl; R² is isopropyl; R¹ is

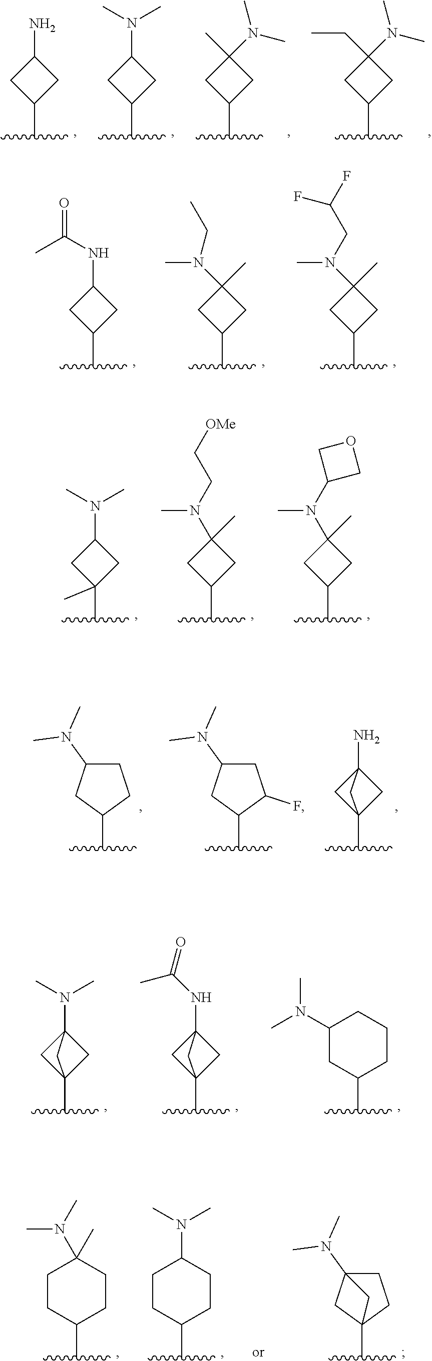

and n is 1, 2, or 3.

In some embodiments, W is N; Y is N; Z is O; Ring A is

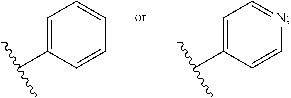 [SMILES: *c1ccccc1 ; "or" ; *c1ccncc1]

### R³ is

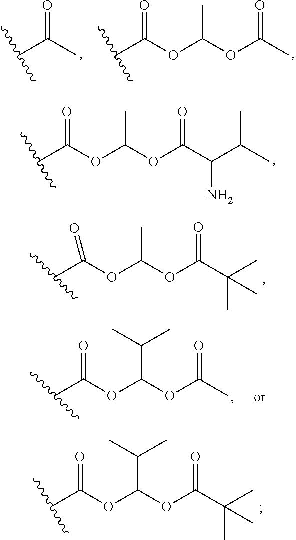

X³ is H, methyl, or ethyl; R² is isopropyl; R¹ is

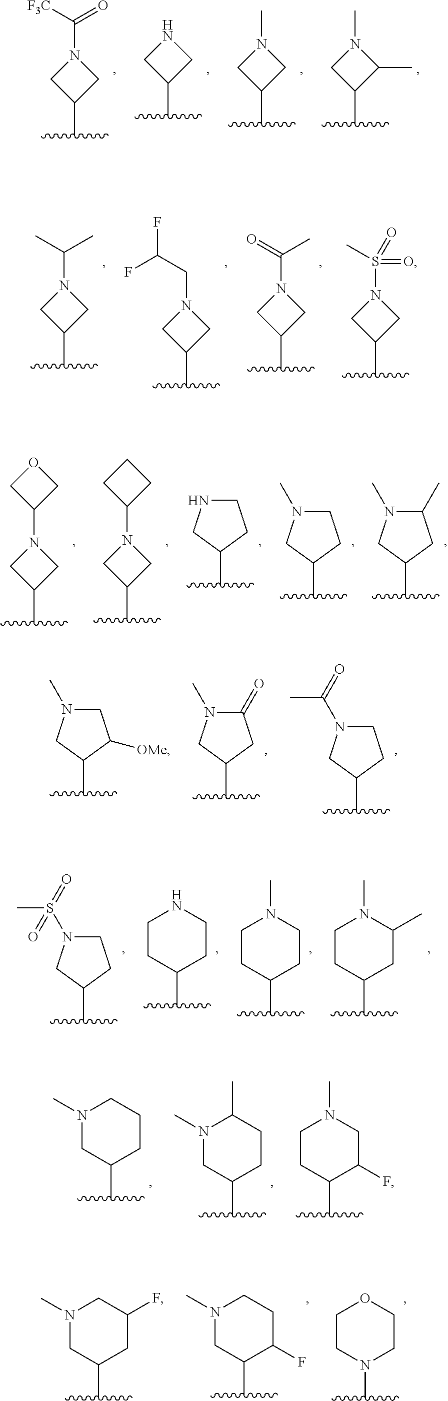

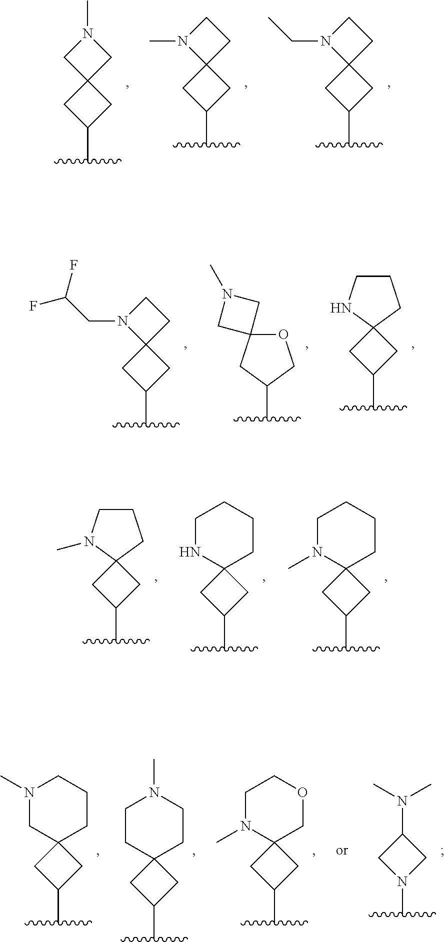

and n is 1, 2, or 3.

In some embodiments, W is N; Y is N; Z is O; Ring A is

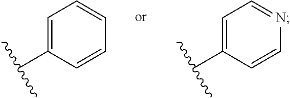 [SMILES: *c1ccccc1 ; "or" ; *c1ccncc1]

### R³ is

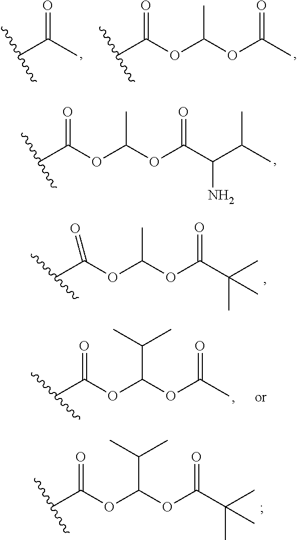

X³ is H, methyl, or ethyl; R² is isopropyl; R¹ is

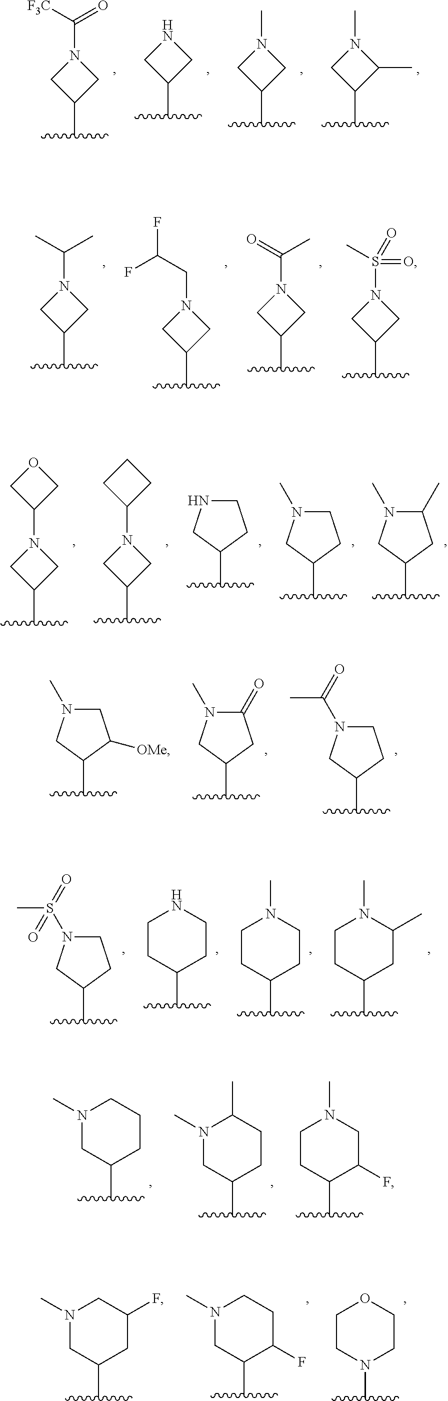

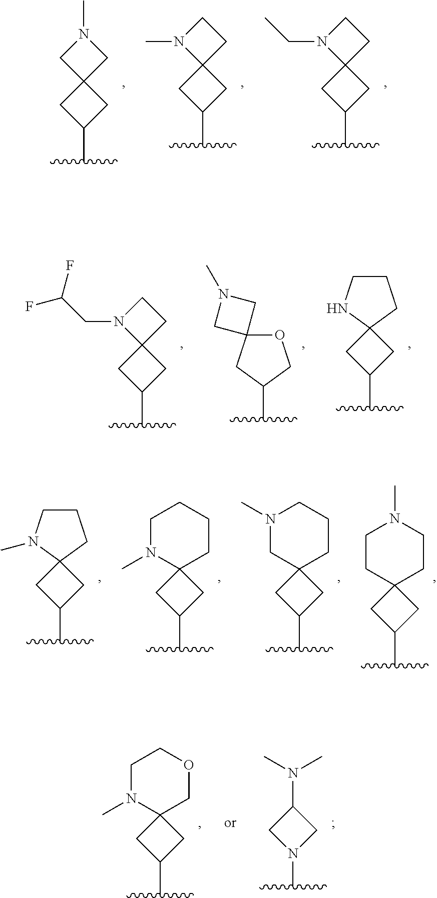

and n is 1, 2, or 3.

In some embodiments, W is N; Y is N; Z is O; Ring A is

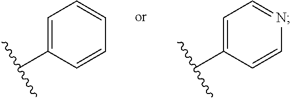 [SMILES: *c1ccccc1 ; "or" ; *c1ccncc1]

### R³ is

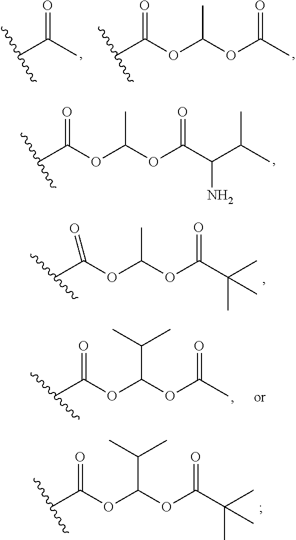

X³ is H, methyl, or ethyl; R² is isopropyl; R¹ is

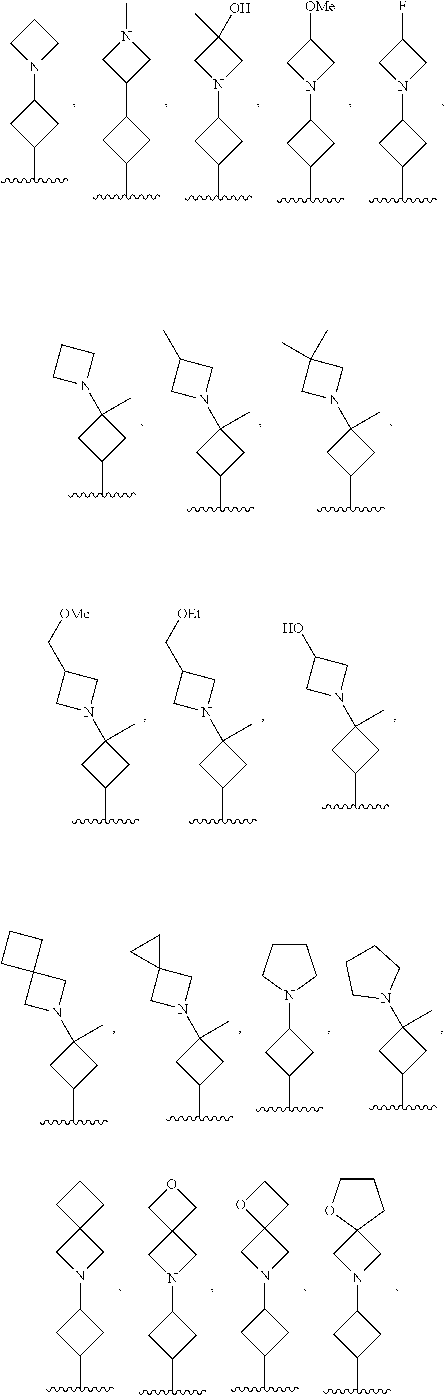

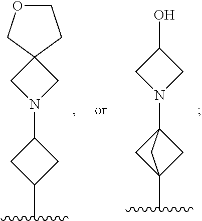 [SMILES: *C1CC(N2CC3(CCOC3)C2)C1 ; *C12CC(N3CC(O)C3)(C1)C2]

and n is 1, 2, or 3.

In some embodiments, W is N, Y is CH, Z is O,

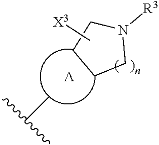

is

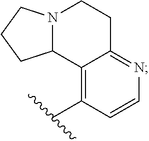 [SMILES: *c1ccnc2c1C1CCCN1CC2]

and n is 1, 2, or 3.

In some embodiments, W is N; Y is CH; Z is O;

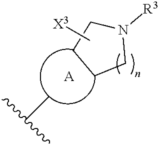

is

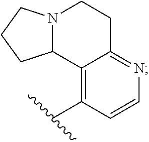 [SMILES: *c1ccnc2c1C1CCCN1CC2]

R² is isopropyl; R¹ is

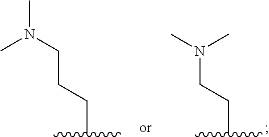

and n is 1, 2, or 3.

In some embodiments, W is N; Y is CH; Z is O;

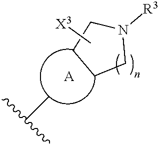

is

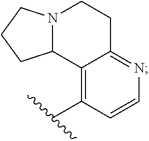 [SMILES: *c1ccnc2c1C1CCCN1CC2]

R² is isopropyl; R¹ is

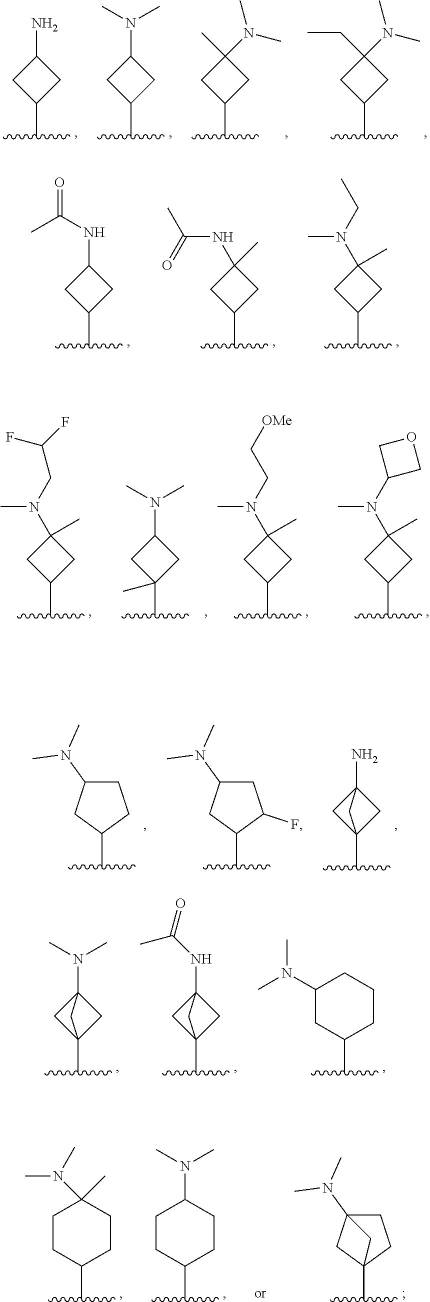

and n is 1, 2, or 3.

In some embodiments, W is N; Y is CH; Z is O;

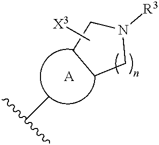

is

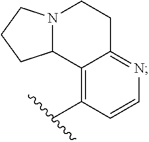 [SMILES: *c1ccnc2c1C1CCCN1CC2]

R² is isopropyl; R¹ is

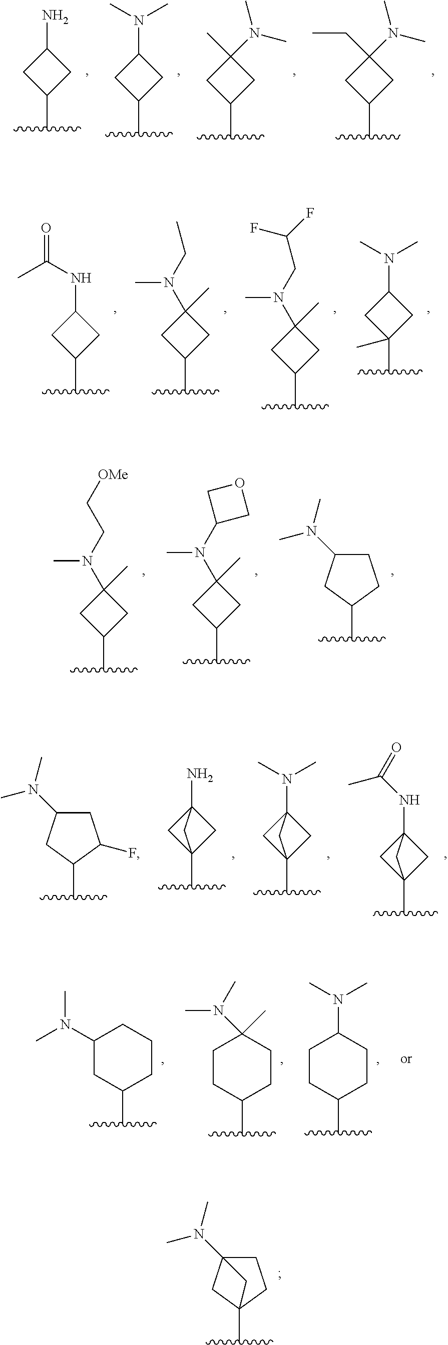

and n is 1, 2, or 3.

In some embodiments, W is N; Y is CH; Z is O;

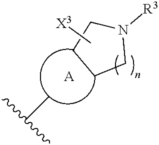

is

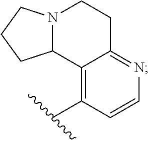 [SMILES: *c1ccnc2c1C1CCCN1CC2]

R² is isopropyl; R¹ is

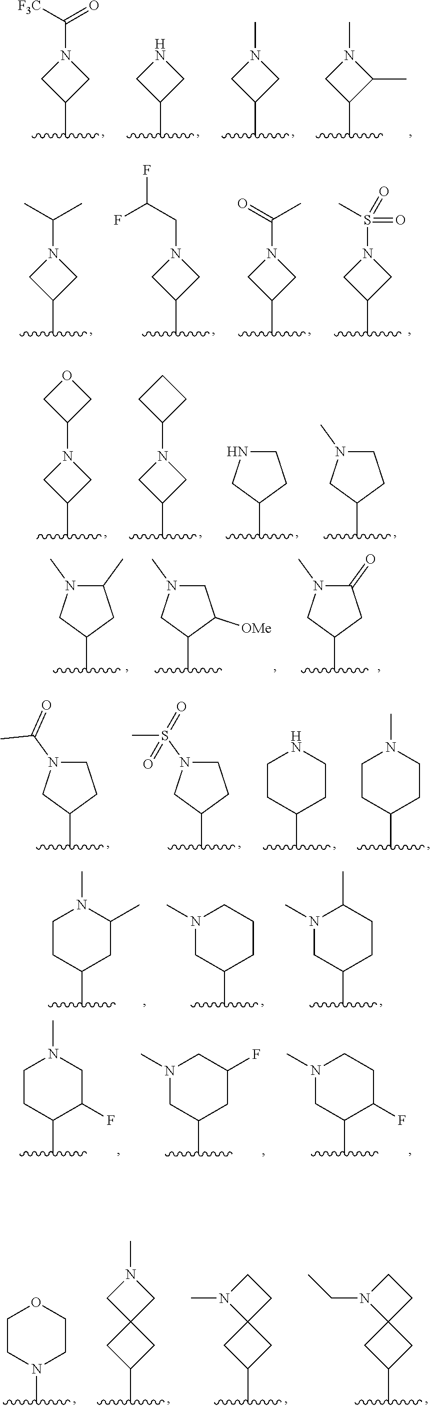

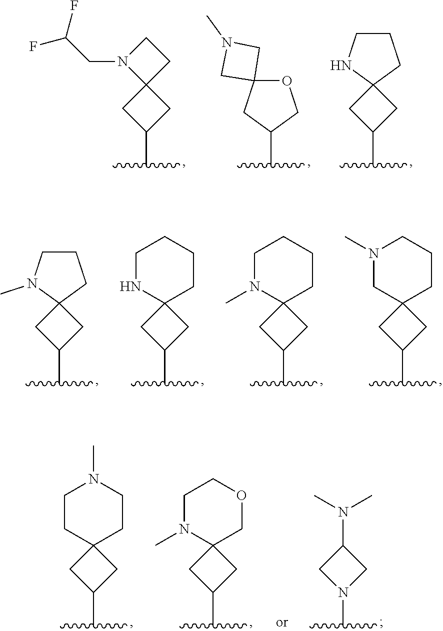

and n is 1, 2, or 3.

In some embodiments W is N; Y is CH; Z is O;

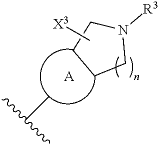

is

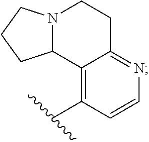 [SMILES: *c1ccnc2c1C1CCCN1CC2]

R² is isopropyl; R¹ is

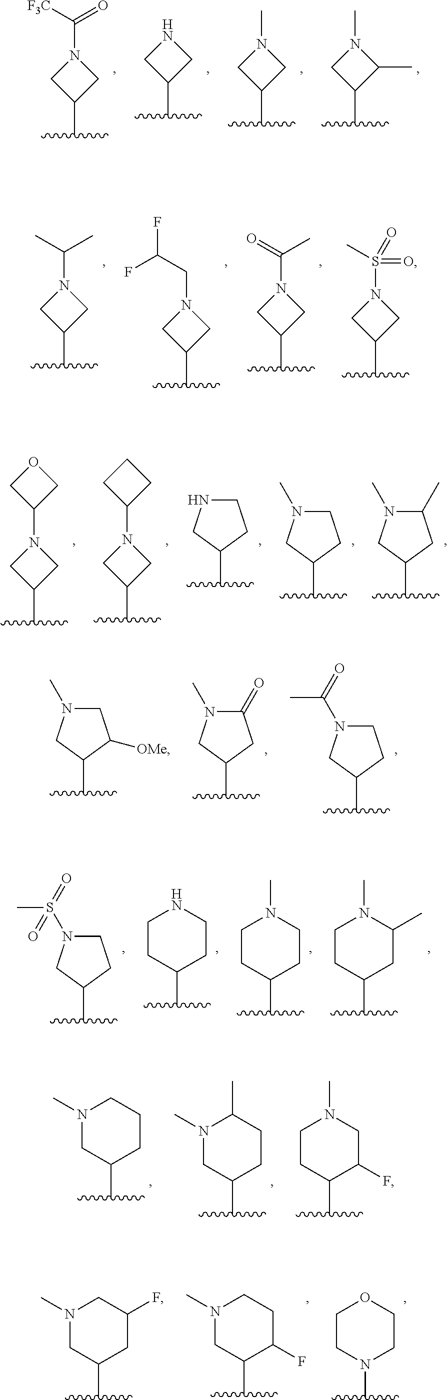

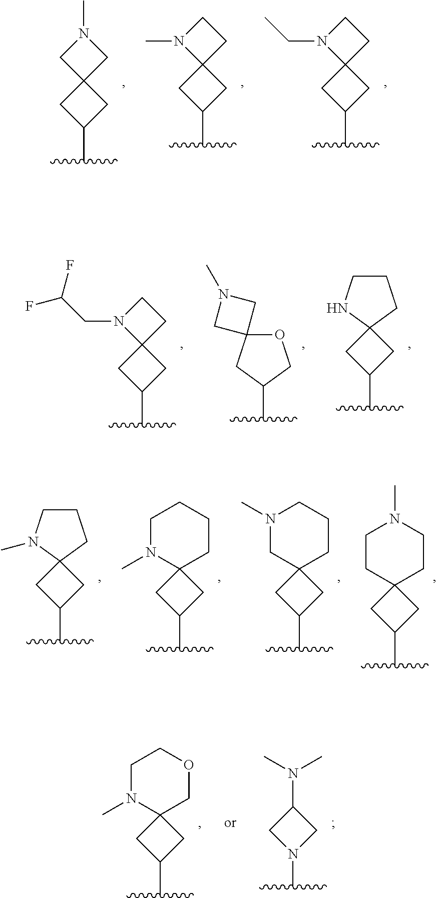

and n is 1, 2, or 3.

In some embodiments, W is N; Y is CH; Z is O;

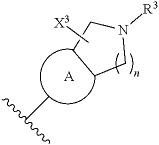

is

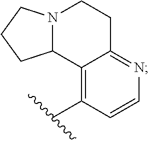 [SMILES: *c1ccnc2c1C1CCCN1CC2]

R² is isopropyl; R¹ is

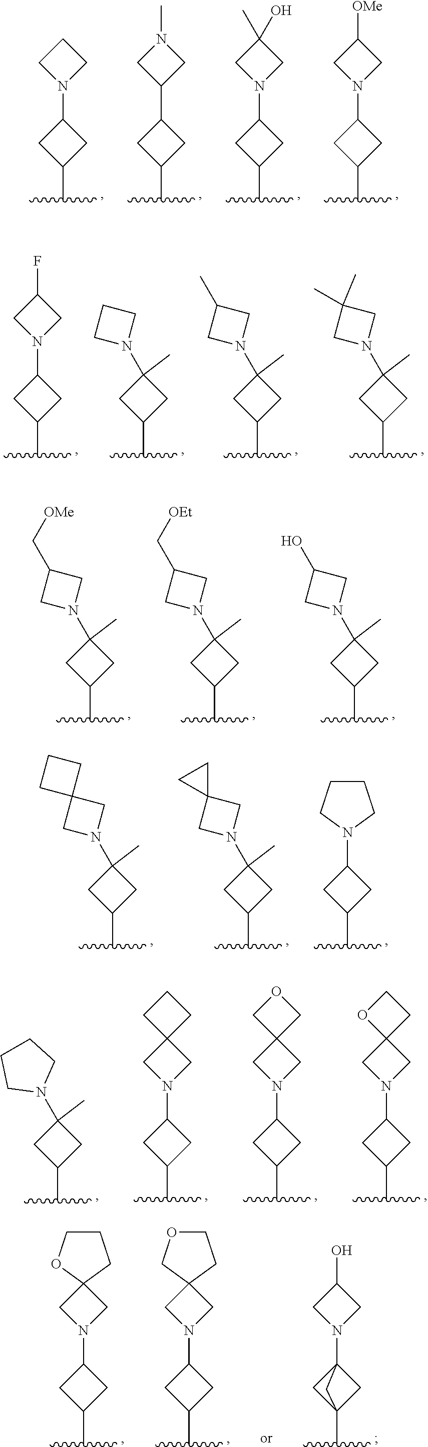

and n is 1, 2, or 3.

In some embodiments, the compound is of Formula I′, I, I-a, I-b, or I-c combined with any of the embodiments described herein.

Any of the groups described above for any variable can be combined with any of the other groups described above, where applicable, for any of the Formulae described herein.

Representative compounds of the present disclosure are shown in the table below.

TABLE 1
Representative Compounds of the Present Disclosure

| Compound | | |
|---|---|---|
| No. | Structure | IUPAC Name |
| 1 | 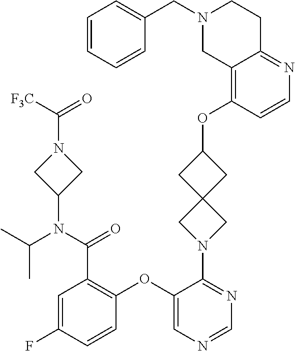 [SMILES: CC(C)N(C(=O)c1cc(F)ccc1Oc1cncnc1N1CC2(CC(Oc3ccnc4c3CN(Cc3ccccc3)CC4)C2)C1)C1CN(C(=O)C(F)(F)F)C1] | 2-((4-(6-((6-Benzyl-5,6,7,8-tetrahydro- 1,6-naphthyridin-4-yl)oxy)-2- azaspiro[3.3]heptan-2-yl)pyrimidin-5- yl)oxy)-5-fluoro-N-isopropyl-N-(1- (2,2,2-trifluoroacetyl)azetidin-3- yl)benzamide |
| 2 | 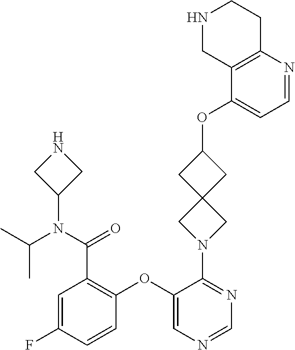 [SMILES: CC(C)N(C(=O)c1cc(F)ccc1Oc1cncnc1N1CC2(CC(Oc3ccnc4c3CNCC4)C2)C1)C1CNC1] | N-(Azetidin-3-yl)-5-fluoro-N-isopropyl- 2-((4-(6-((5,6,7,8-tetrahydro-1,6- naphthyridin-4-yl)oxy)-2- azaspiro[3.3]heptan-2-yl)pyrimidin-5- yl)oxy)benzamide |
| 3 | 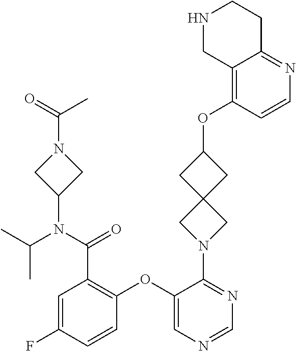 [SMILES: CC(=O)N1CC(N(C(=O)c2cc(F)ccc2Oc2cncnc2N2CC3(CC(Oc4ccnc5c4CNCC5)C3)C2)C(C)C)C1] | N-(1-Acetylazetidin-3-yl)-5-fluoro-N- isopropyl-2-((4-(6-((5,6,7,8-tetrahydro- 1,6-naphthyridin-4-yl)oxy)-2- azaspiro[3.3]heptan-2-yl)pyrimidin-5- yl)oxy)benzamide |
| 4 | 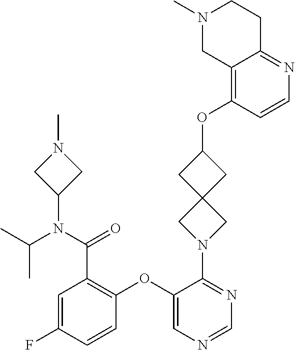 [SMILES: CC(C)N(C(=O)c1cc(F)ccc1Oc1cncnc1N1CC2(CC(Oc3ccnc4c3CN(C)CC4)C2)C1)C1CN(C)C1] | 5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-(1-methylazetidin-3- yl)benzamide |
| 5 | 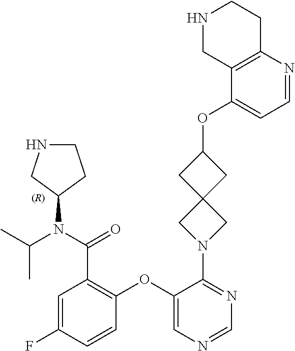 | (R)-5-Fluoro-N-isopropyl-N-(pyrrolidin- 3-yl)-2-((4-(6-((5,6,7,8-tetrahydro-1,6- naphthyridin-4-yl)oxy)-2- azaspiro[3.3]heptan-2-yl)pyrimidin-5- yl)oxy)benzamide |
| 6 | 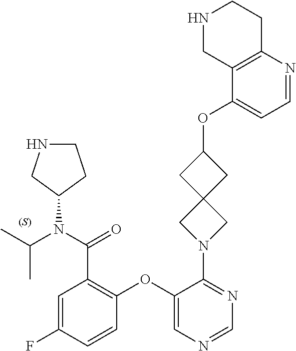 | (S)-5-Fluoro-N-isopropyl-N-(pyrrolidin- 3-yl)-2-((4-(6-((5,6,7,8-tetrahydro-1,6- naphthyridin-4-yl)oxy)-2- azaspiro[3.3]heptan-2-yl)pyrimidin-5- yl)oxy)benzamide |
| 7 | 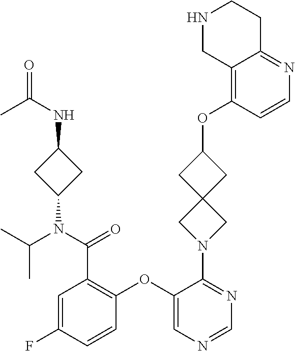 [SMILES: CC(=O)N[C@H]1C[C@H](N(C(=O)c2cc(F)ccc2Oc2cncnc2N2CC3(CC(Oc4ccnc5c4CNCC5)C3)C2)C(C)C)C1] | N-((1r,3r)-3-Acetamidocyclobutyl)-5- fluoro-N-isopropyl-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 8 | 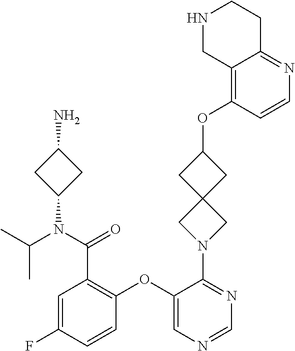 [SMILES: CC(C)N(C(=O)c1cc(F)ccc1Oc1cncnc1N1CC2(CC(Oc3ccnc4c3CNCC4)C2)C1)[C@H]1C[C@@H](N)C1] | N-((1s,3s)-3-Aminocyclobutyl)-5- fluoro-N-isopropyl-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 9 | 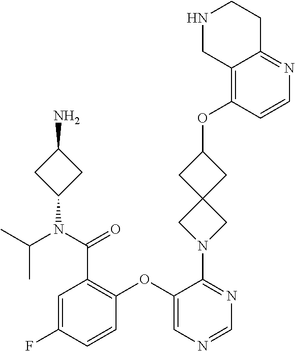 [SMILES: CC(C)N(C(=O)c1cc(F)ccc1Oc1cncnc1N1CC2(CC(Oc3ccnc4c3CNCC4)C2)C1)[C@H]1C[C@H](N)C1] | N-((1r,3r)-3-Aminocyclobutyl)-5- fluoro-N-isopropyl-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 10 | 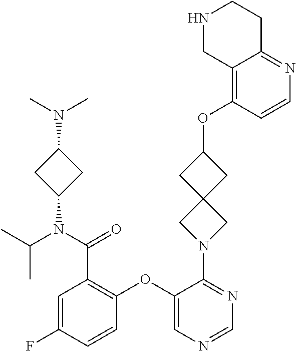 [SMILES: CC(C)N(C(=O)c1cc(F)ccc1Oc1cncnc1N1CC2(CC(Oc3ccnc4c3CNCC4)C2)C1)[C@H]1C[C@@H](N(C)C)C1] | N-((1s,3s)-3- (Dimethylamino)cyclobutyl)-5-fluoro- N-isopropyl-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 11 | 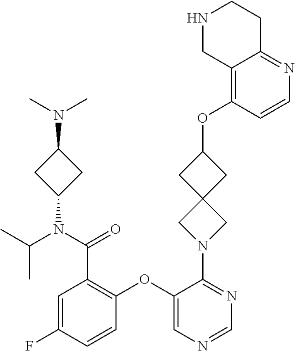 [SMILES: CC(C)N(C(=O)c1cc(F)ccc1Oc1cncnc1N1CC2(CC(Oc3ccnc4c3CNCC4)C2)C1)[C@H]1C[C@H](N(C)C)C1] | N-((1r,3r)-3- (Dimethylamino)cyclobutyl)-5-fluoro- N-isopropyl-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 12 | 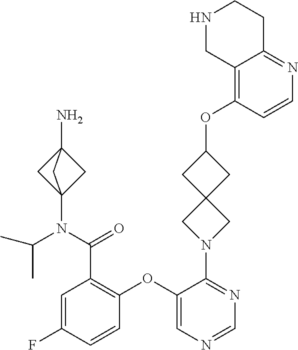 [SMILES: CC(C)N(C(=O)c1cc(F)ccc1Oc1cncnc1N1CC2(CC(Oc3ccnc4c3CNCC4)C2)C1)C12CC(N)(C1)C2] | N-(3-Aminobicyclo[1.1.1]pentan-1-yl)- 5-fluoro-N-isopropyl-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 13 | 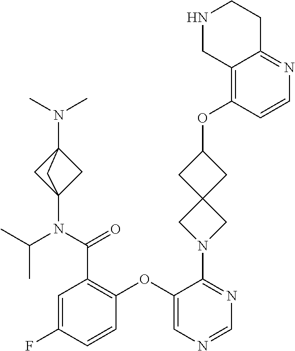 [SMILES: CC(C)N(C(=O)c1cc(F)ccc1Oc1cncnc1N1CC2(CC(Oc3ccnc4c3CNCC4)C2)C1)C12CC(N(C)C)(C1)C2] | N-(3- (Dimethylamino)bicyclo[1.1.1]pentan- 1-yl)-5-fluoro-N-isopropyl-2-((4-(6- ((5,6,7,8-tetrahydro-1,6-naphthyridin-4- yl)oxy)-2-azaspiro[3.3]heptan-2- yl)pyrimidin-5-yl)oxy)benzamide |
| 14 | 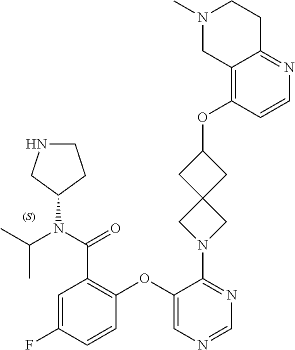 | (S)-5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-(pyrrolidin-3-yl)benzamide |
| 15 | 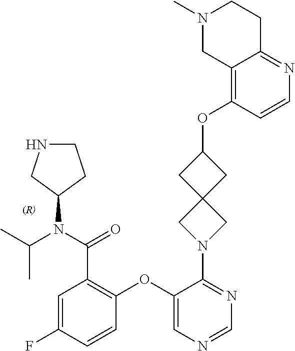 | (R)-5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-(pyrrolidin-3-yl)benzamide |
| 16 | 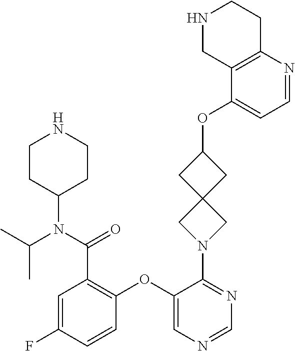 [SMILES: CC(C)N(C(=O)c1cc(F)ccc1Oc1cncnc1N1CC2(CC(Oc3ccnc4c3CNCC4)C2)C1)C1CCNCC1] | 5-Fluoro-N-isopropyl-N-(piperidin-4- yl)-2-((4-(6-((5,6,7,8-tetrahydro-1,6- naphthyridin-4-yl)oxy)-2- azaspiro[3.3]heptan-2-yl)pyrimidin-5- yl)oxy)benzamide |
| 17 | 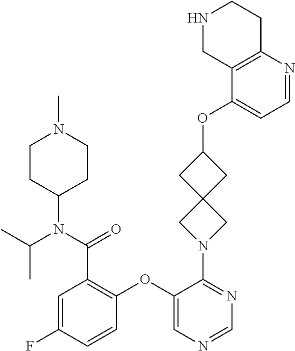 [SMILES: CC(C)N(C(=O)c1cc(F)ccc1Oc1cncnc1N1CC2(CC(Oc3ccnc4c3CNCC4)C2)C1)C1CCN(C)CC1] | 5-Fluoro-N-isopropyl-N-(1- methylpiperidin-4-yl)-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 18 | 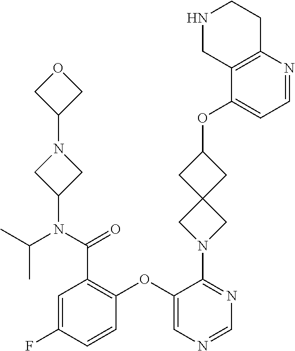 [SMILES: CC(C)N(C(=O)c1cc(F)ccc1Oc1cncnc1N1CC2(CC(Oc3ccnc4c3CNCC4)C2)C1)C1CN(C2COC2)C1] | 5-Fluoro-N-isopropyl-N-(1-(oxetan-3- yl)azetidin-3-yl)-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 19 | 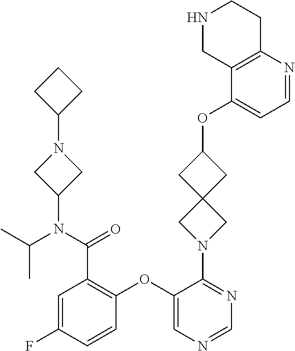 [SMILES: CC(C)N(C(=O)c1cc(F)ccc1Oc1cncnc1N1CC2(CC(Oc3ccnc4c3CNCC4)C2)C1)C1CN(C2CCC2)C1] | N-(1-Cyclobutylazetidin-3-yl)-5-fluoro- N-isopropyl-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 20 | 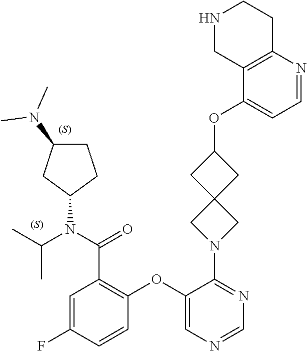 | N-((1S,3S)-3-(Dimethylamino) cyclopentyl)-5-fluoro-N-isopropyl-2- ((4-(6-((5,6,7,8-tetrahydro-1,6- naphthyridin-4-yl)oxy)-2-azaspiro[3.3] heptan-2-yl)pyrimidin-5- yl)oxy)benzamide |
| 21 | 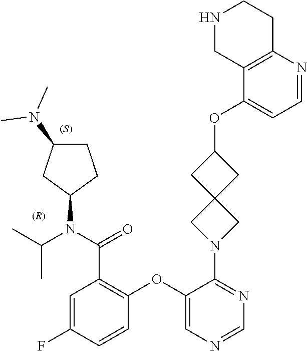 | N-((1R,3S)-3- (Dimethylamino)cyclopentyl)-5-fluoro- N-isopropyl-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 22 | 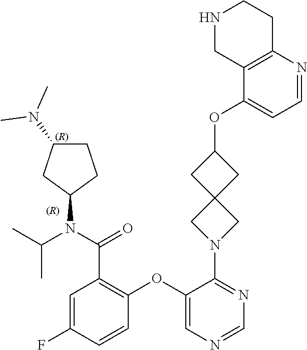 | N-((1R,3R)-3- (Dimethylamino)cyclopentyl)-5-fluoro- N-isopropyl-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 23 | 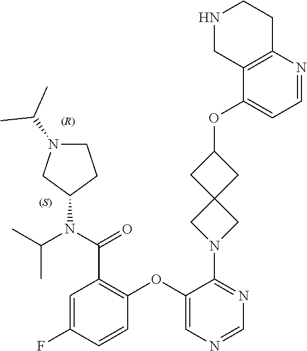 | N-((1S,3R)-3- (Dimethylamino)cyclopentyl)-5-fluoro- N-isopropyl-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 24 | 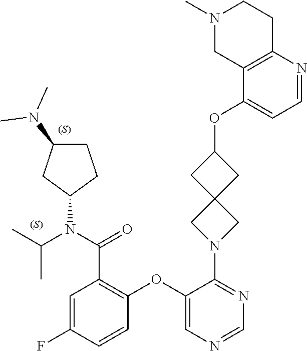 | N-((1S,3S)-3- (Dimethylamino)cyclopentyl)-5-fluoro- N-isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 25 | 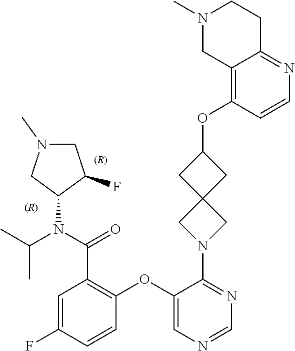 | 5-Fluoro-N-((3R,4R)-4-fluoro-1- methylpyrrolidin-3-yl)-N-isopropyl-2- ((4-(6-((6-methyl-5,6,7,8-tetrahydro- 1,6-naphthyridin-4-yl)oxy)-2- azaspiro[3.3]heptan-2-yl)pyrimidin-5- yl)oxy)benzamide |
| 26 | 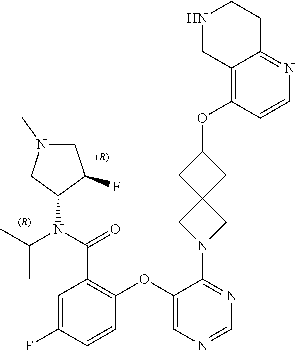 | 5-Fluoro-N-((3R,4R)-4-fluoro-1- methylpyrrolidin-3-yl)-N-isopropyl-2- ((4-(6-((5,6,7,8-tetrahydro-1,6- naphthyridin-4-yl)oxy)-2- azaspiro[3.3]heptan-2-yl)pyrimidin-5- yl)oxy)benzamide |
| 27 | 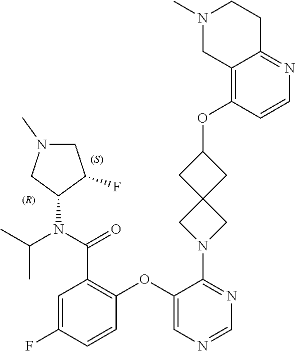 | 5-Fluoro-N-((3R,4S)-4-fluoro-1- methylpyrrolidin-3-yl)-N-isopropyl-2- ((4-(6-((6-methyl-5,6,7,8-tetrahydro- 1,6-naphthyridin-4-yl)oxy)-2- azaspiro[3.3]heptan-2-yl)pyrimidin-5- yl)oxy)benzamide |
| 28 | 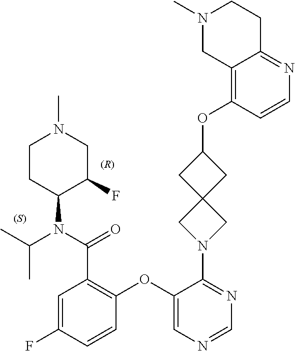 | 5-Fluoro-N-((3R,4S)-3-fluoro-1- methylpiperidin-4-yl)-N-isopropyl-2- ((4-(6-((6-methyl-5,6,7,8-tetrahydro- 1,6-naphthyridin-4-yl)oxy)-2-azaspiro [3.3]heptan-2-yl)pyrimidin-5-yl)oxy) benzamide |
| 29 | 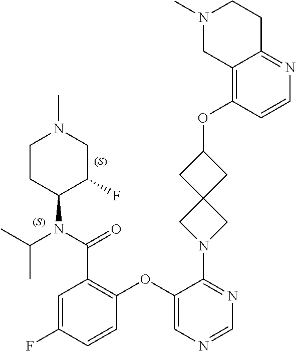 | 5-Fluoro-N-((3S,4S)-3-fluoro-1- methylpiperidin-4-yl)-N-isopropyl-2- ((4-(6-((6-methyl-5,6,7,8-tetrahydro- 1,6-naphthyridin-4-yl)oxy)-2- azaspiro[3.3]heptan-2-yl)pyrimidin-5- yl)oxy)benzamide |
| 30 | 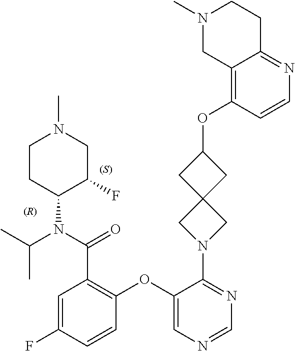 | 5-Fluoro-N-((3S,4R)-3-fluoro-1- methylpiperidin-4-yl)-N-isopropyl-2- ((4-(6-((6-methyl-5,6,7,8-tetrahydro- 1,6-naphthyridin-4-yl)oxy)-2- azaspiro[3.3]heptan-2-yl)pyrimidin-5- yl)oxy)benzamide |
| 31 | 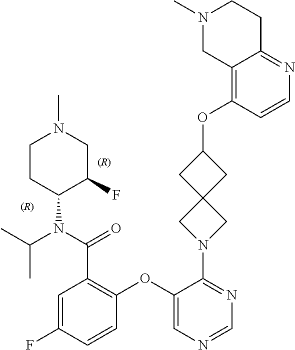 | 5-Fluoro-N-((3R,4R)-3-fluoro-1- methylpiperidin-4-yl)-N-isopropyl-2- ((4-(6-((6-methyl-5,6,7,8-tetrahydro- 1,6-naphthyridin-4-yl)oxy)-2- azaspiro[3.3]heptan-2-yl)pyrimidin-5- yl)oxy)benzamide |
| 32 | 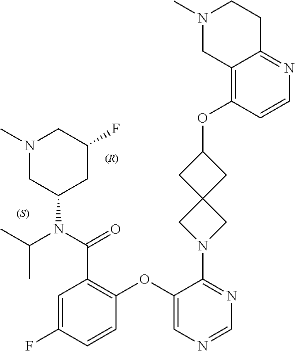 | 5-Fluoro-N-((3S,5R)-5-fluoro-1- methylpiperidin-3-yl)-N-isopropyl-2- ((4-(6-((6-methyl-5,6,7,8-tetrahydro- 1,6-naphthyridin-4-yl)oxy)-2- azaspiro[3.3]heptan-2-yl)pyrimidin-5- yl)oxy)benzamide |
| 33 | 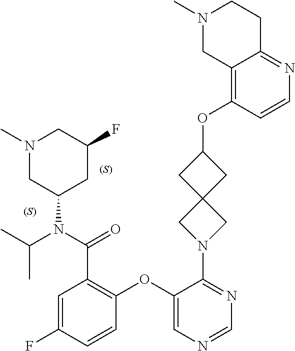 | 5-Fluoro-N-((3S,5S)-5-fluoro-1- methylpiperidin-3-yl)-N-isopropyl-2- ((4-(6-((6-methyl-5,6,7,8-tetrahydro- 1,6-naphthyridin-4-yl)oxy)-2- azaspiro[3.3]heptan-2-yl)pyrimidin-5- yl)oxy)benzamide |
| 34 | 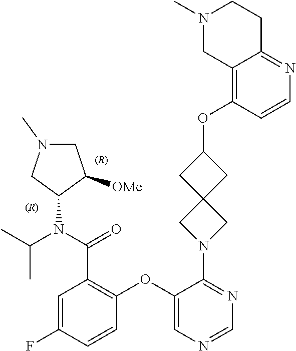 | 5-Fluoro-N-isopropyl-N-((3R,4R)-4- methoxy-1-methylpyrrolidin-3-yl)-2- ((4-(6-((6-methyl-5,6,7,8-tetrahydro- 1,6-naphthyridin-4-yl)oxy)-2- azaspiro[3.3]heptan-2-yl)pyrimidin-5- yl)oxy)benzamide |
| 35 | 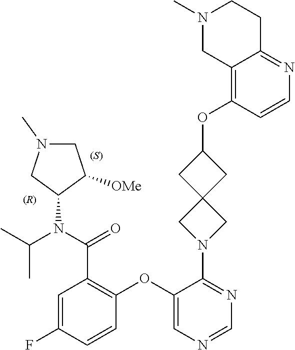 | 5-Fluoro-N-isopropyl-N-((3R,4S)-4- methoxy-1-methylpyrrolidin-3-yl)-2- ((4-(6-((6-methyl-5,6,7,8-tetrahydro- 1,6-naphthyridin-4-yl)oxy)-2- azaspiro[3.3]heptan-2-yl)pyrimidin-5- yl)oxy)benzamide |
| 36 | 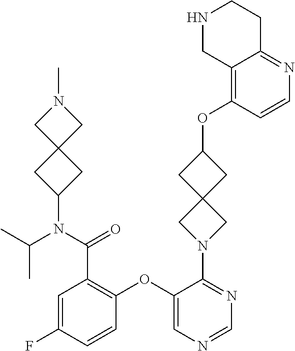 [SMILES: CC(C)N(C(=O)c1cc(F)ccc1Oc1cncnc1N1CC2(CC(Oc3ccnc4c3CNCC4)C2)C1)C1CC2(C1)CN(C)C2] | 5-Fluoro-N-isopropyl-N-(2-methyl-2- azaspiro[3.3]heptan-6-yl)-2-((4-(6- ((5,6,7,8-tetrahydro-1,6-naphthyridin-4- yl)oxy)-2-azaspiro[3.3]heptan-2- yl)pyrimidin-5-yl)oxy)benzamide |
| 37 | 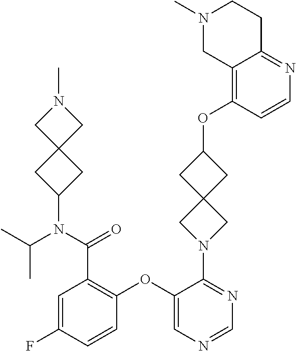 [SMILES: CC(C)N(C(=O)c1cc(F)ccc1Oc1cncnc1N1CC2(CC(Oc3ccnc4c3CN(C)CC4)C2)C1)C1CC2(C1)CN(C)C2] | 5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-(2-methyl-2- azaspiro[3.3]heptan-6-yl)benzamide |
| 38 | 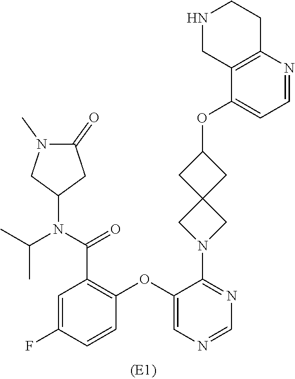 [SMILES: CC(C)N(C(=O)c1cc(F)ccc1Oc1cncnc1N1CC2(CC(Oc3ccnc4c3CNCC4)C2)C1)C1CC(=O)N(C)C1] | (E1)-5-Fluoro-N-isopropyl-N-(1-methyl- 5-oxopyrrolidin-3-yl)-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 39 | 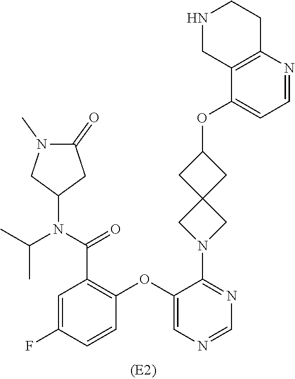 [SMILES: CC(C)N(C(=O)c1cc(F)ccc1Oc1cncnc1N1CC2(CC(Oc3ccnc4c3CNCC4)C2)C1)C1CC(=O)N(C)C1] | (E2)-5-Fluoro-N-isopropyl-N-(1-methyl- 5-oxopyrrolidin-3-yl)-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 40 | 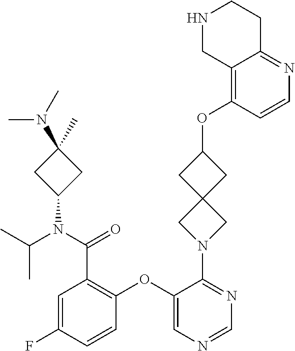 [SMILES: CC(C)N(C(=O)c1cc(F)ccc1Oc1cncnc1N1CC2(CC(Oc3ccnc4c3CNCC4)C2)C1)[C@H]1C[C@](C)(N(C)C)C1] | N-((1r,3r)-3-(Dimethylamino)-3- methylcyclobutyl)-5-fluoro-N- isopropyl-2-((4-(6-((5,6,7,8-tetrahydro- 1,6-naphthyridin-4-yl)oxy)-2- azaspiro[3.3]heptan-2-yl)pyrimidin-5- yl)oxy)benzamide |
| 41 | 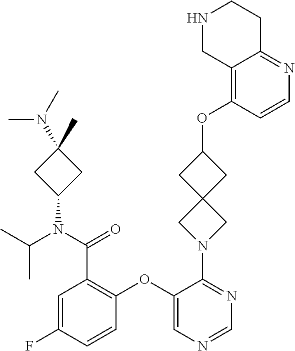 [SMILES: CC(C)N(C(=O)c1cc(F)ccc1Oc1cncnc1N1CC2(CC(Oc3ccnc4c3CNCC4)C2)C1)[C@H]1C[C@@](C)(N(C)C)C1] | N-((1s,3s)-3-(Dimethylamino)-3- methylcyclobutyl)-5-fluoro-N- isopropyl-2-((4-(6-((5,6,7,8-tetrahydro- 1,6-naphthyridin-4-yl)oxy)-2- azaspiro[3.3]heptan-2-yl)pyrimidin-5- yl)oxy)benzamide |
| 42 | 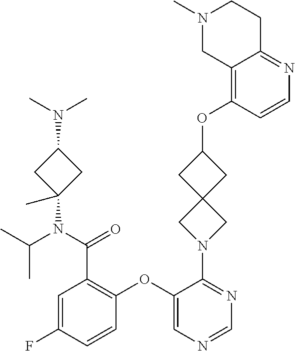 [SMILES: CC(C)N(C(=O)c1cc(F)ccc1Oc1cncnc1N1CC2(CC(Oc3ccnc4c3CN(C)CC4)C2)C1)[C@]1(C)C[C@H](N(C)C)C1] | N-((1s,3s)-3-(Dimethylamino)-1- methylcyclobutyl)-5-fluoro-N- isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 43 | 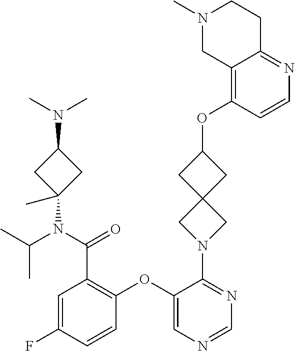 [SMILES: CC(C)N(C(=O)c1cc(F)ccc1Oc1cncnc1N1CC2(CC(Oc3ccnc4c3CN(C)CC4)C2)C1)[C@]1(C)C[C@@H](N(C)C)C1] | N-((1r,3r)-3-(Dimethylamino)-1- methylcyclobutyl)-5-fluoro-N- isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 44 | 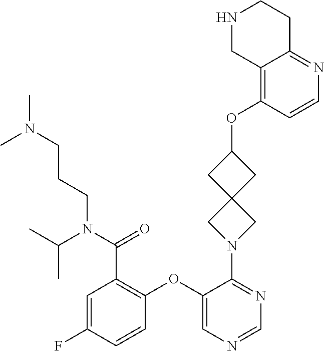 [SMILES: CC(C)N(CCCN(C)C)C(=O)c1cc(F)ccc1Oc1cncnc1N1CC2(CC(Oc3ccnc4c3CNCC4)C2)C1] | N-(3-(Dimethylamino)propyl)-5-fluoro- N-isopropyl-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 45 | 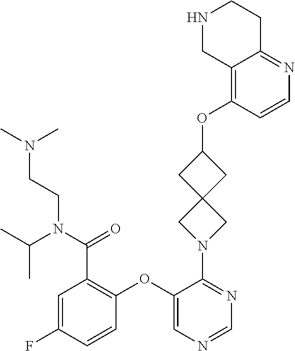 [SMILES: CC(C)N(CCN(C)C)C(=O)c1cc(F)ccc1Oc1cncnc1N1CC2(CC(Oc3ccnc4c3CNCC4)C2)C1] | N-(2-(Dimethylamino)ethyl)-5-fluoro- N-isopropyl-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 46 | 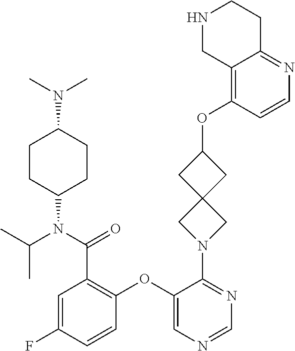 | N-((1s,4s)-4- (Dimethylamino)cyclohexyl)-5-fluoro- N-isopropyl-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 47 | 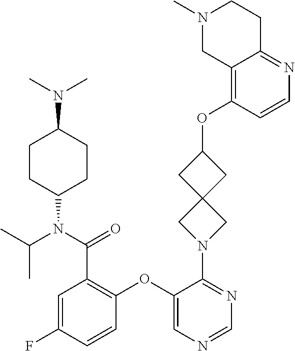 | N-((1r,4r)-4- (Dimethylamino)cyclohexyl)-5-fluoro- N-isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 48 | 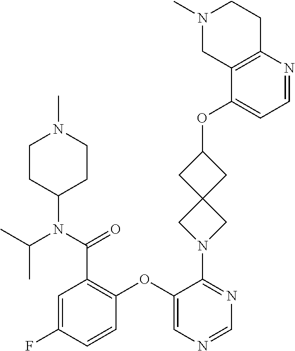 [SMILES: CC(C)N(C(=O)c1cc(F)ccc1Oc1cncnc1N1CC2(CC(Oc3ccnc4c3CN(C)CC4)C2)C1)C1CCN(C)CC1] | 5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-(1-methylpiperidin-4- yl)benzamide |
| 49 | 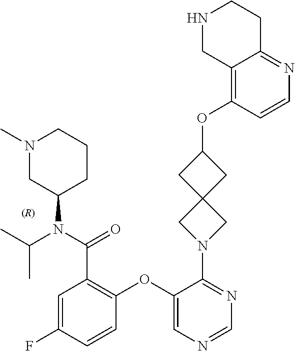 | (R)-5-Fluoro-N-isopropyl-N-(1- methylpiperidin-3-yl)-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 50 | 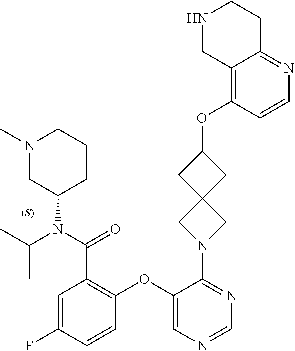 | (S)-5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-(1-methylpiperidin-3- yl)benzamide |
| 51 | 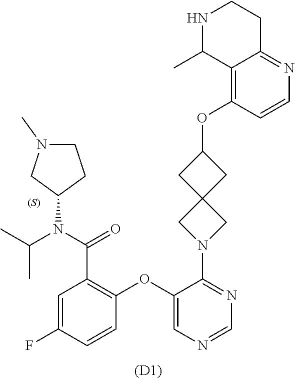 | (D1)-5-Fluoro-N-isopropyl-2-((4-(6-((5- methyl-5,6,7,8-tetrahydro-1,6- naphthyridin-4-yl)oxy)-2- azaspiro[3.3]heptan-2-yl)pyrimidin-5- yl)oxy)-N-((S)-1-methylpyrrolidin-3- yl)benzamide |
| 52 | 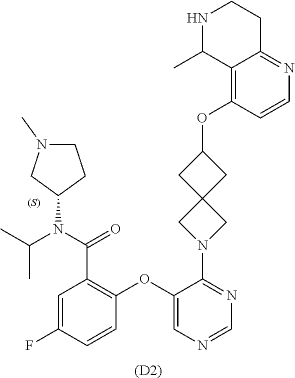 | (D2)-5-Fluoro-N-isopropyl-2-((4-(6-((5- methyl-5,6,7,8-tetrahydro-1,6- naphthyridin-4-yl)oxy)-2- azaspiro[3.3]heptan-2-yl)pyrimidin-5- yl)oxy)-N-((S)-1-methylpyrrolidin-3- yl)benzamide |
| 53 | 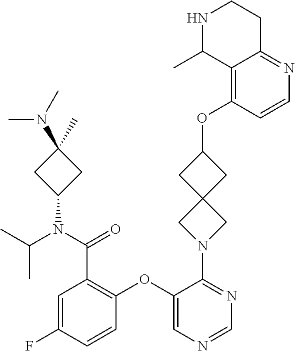 [SMILES: CC1NCCc2nccc(OC3CC4(C3)CN(c3ncncc3Oc3ccc(F)cc3C(=O)N(C(C)C)[C@H]3C[C@](C)(N(C)C)C3)C4)c21] | (D1)-N-((1r,3r)-3-(Dimethylamino)-3- methylcyclobutyl)-5-fluoro-N- isopropyl-2-((4-(6-((5-methyl-5,6,7,8- tetrahydro-1,6-naphthyridin-4-yl)oxy)- 2-azaspiro[3.3]heptan-2-yl)pyrimidin-5- yl)oxy)benzamide |
| | (Isomer 1) | |
| 54 | 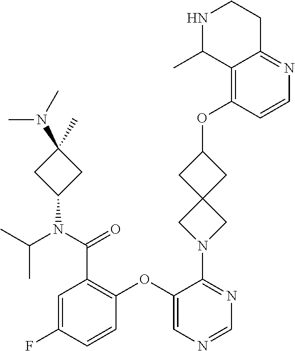 [SMILES: CC1NCCc2nccc(OC3CC4(C3)CN(c3ncncc3Oc3ccc(F)cc3C(=O)N(C(C)C)[C@H]3C[C@](C)(N(C)C)C3)C4)c21] | (D2)-N-((1r,3r)-3-(Dimethylamino)-3- methylcyclobutyl)-5-fluoro-N- isopropyl-2-((4-(6-((5-methyl-5,6,7,8- tetrahydro-1,6-naphthyridin-4-yl)oxy)- 2-azaspiro[3.3]heptan-2-yl)pyrimidin-5- yl)oxy)benzamide |
| | (Isomer 2) | |
| 55 | 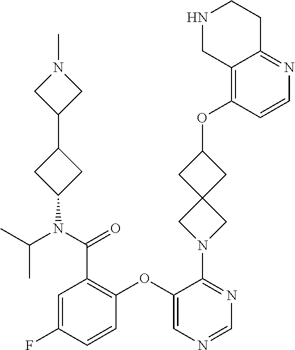 | (D1)-5-Fluoro-N-isopropyl-N-(3-(1- methylazetidin-3-yl)cyclobutyl)-2-((4- (6-((5,6,7,8-tetrahydro-1,6- naphthyridin-4-yl)oxy)-2- azaspiro[3.3]heptan-2-yl)pyrimidin-5- yl)oxy)benzamide |
| | (D1) | |
| 56 | 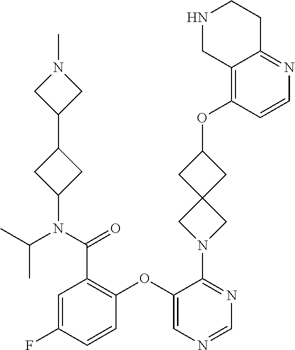 [SMILES: CC(C)N(C(=O)c1cc(F)ccc1Oc1cncnc1N1CC2(CC(Oc3ccnc4c3CNCC4)C2)C1)C1CC(C2CN(C)C2)C1] | (D2)-5-Fluoro-N-isopropyl-N-(3-(1- methylazetidin-3-yl)cyclobutyl)-2-((4- (6-((5,6,7,8-tetrahydro-1,6- naphthyridin-4-yl)oxy)-2- azaspiro[3.3]heptan-2-yl)pyrimidin-5- yl)oxy)benzamide |
| | (D2) | |
| 57 | 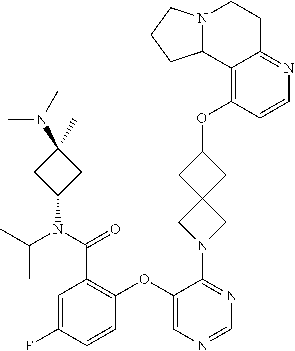 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(N(C)C)C1] | (D1)-N-((1r,3r)-3-(Dimethylamino)-3- methylcyclobutyl)-5-fluoro-2-((4-(6- ((5,6,8,9,10,10a-hexahydropyrrolo[2,1- f][1,6]naphthyridin-1-yl)oxy)-2- azaspiro[3.3]heptan-2-yl)pyrimidin-5- yl)oxy)-N-isopropylbenzamide |
| | (Isomer 1) | |
| 58 | 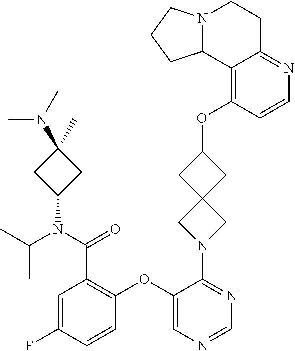 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(N(C)C)C1] | (D2)-N-((1r,3r)-3-(Dimethylamino)-3- methylcyclobutyl)-5-fluoro-2-((4-(6- ((5,6,8,9,10,10a-hexahydropyrrolo[2,1- f][1,6]naphthyridin-1-yl)oxy)-2- azaspiro[3.3]heptan-2-yl)pyrimidin-5- yl)oxy)-N-isopropylbenzamide |
| | (Isomer 2) | |
| 59 | 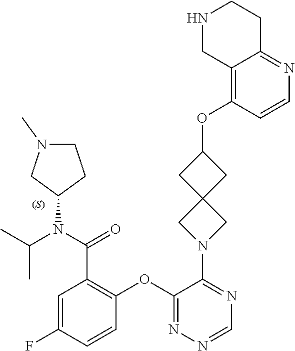 | (S)-5-Fluoro-N-isopropyl-N-(1- methylpyrrolidin-3-yl)-2-((5-(6- ((5,6,7,8-tetrahydro-1,6-naphthyridin-4- yl)oxy)-2-azaspiro[3.3]heptan-2-yl)- 1,2,4-triazin-6-yl)oxy)benzamide |
| 60 | 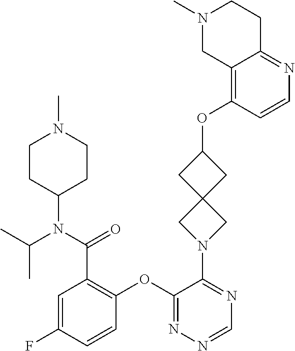 [SMILES: CC(C)N(C(=O)c1cc(F)ccc1Oc1nncnc1N1CC2(CC(Oc3ccnc4c3CN(C)CC4)C2)C1)C1CCN(C)CC1] | 5-Fluoro-N-isopropyl-2-((5-(6-((6- methyl-5,6,7,8-tetrahydro-1,6- naphthyridin-4-yl)oxy)-2- azaspiro[3.3]heptan-2-yl)-1,2,4-triazin- 6-yl)oxy)-N-(1-methylpiperidin-4- yl)benzamide |
| 61 | 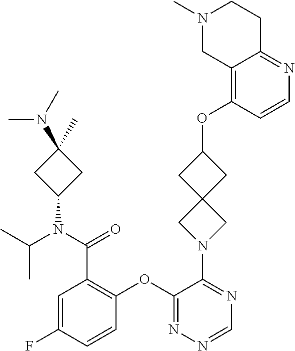 [SMILES: CC(C)N(C(=O)c1cc(F)ccc1Oc1nncnc1N1CC2(CC(Oc3ccnc4c3CN(C)CC4)C2)C1)[C@H]1C[C@](C)(N(C)C)C1] | N-((1r,3r)-3-(Dimethylamino)-3- methylcyclobutyl)-5-fluoro-N- isopropyl-2-((5-(6-((6-methyl-5,6,7,8- tetrahydro-1,6-naphthyridin-4-yl)oxy)- 2-azaspiro[3.3]heptan-2-yl)-1,2,4- triazin-6-yl)oxy)benzamide |
| 62 | 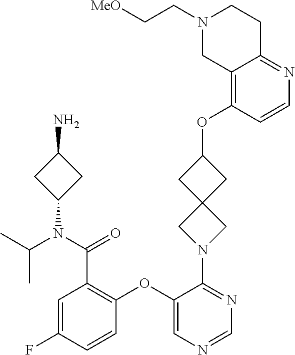 [SMILES: COCCN1CCc2nccc(OC3CC4(C3)CN(c3ncncc3Oc3ccc(F)cc3C(=O)N(C(C)C)[C@H]3C[C@H](N)C3)C4)c2C1] | N-((1r,3r)-3-Aminocyclobutyl)-5- fluoro-N-isopropyl-2-((4-(6-((6-(2- methoxyethyl)-5,6,7,8-tetrahydro-1,6- naphthyridin-4-yl)oxy)-2- azaspiro[3.3]heptan-2-yl)pyrimidin-5- yl)oxy)benzamide |
| 63 | 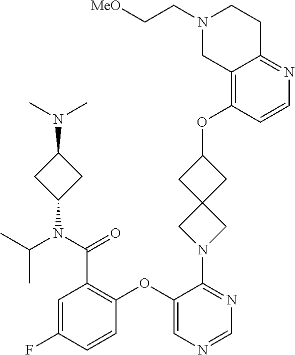 [SMILES: COCCN1CCc2nccc(OC3CC4(C3)CN(c3ncncc3Oc3ccc(F)cc3C(=O)N(C(C)C)[C@H]3C[C@H](N(C)C)C3)C4)c2C1] | N-((1r,3r)-3- (Dimethylamino)cyclobutyl)-5-fluoro- N-isopropyl-2-((4-(6-((6-(2- methoxyethyl)-5,6,7,8-tetrahydro-1,6- naphthyridin-4-yl)oxy)-2- azaspiro[3.3]heptan-2-yl)pyrimidin-5- yl)oxy)benzamide |
| 64 | 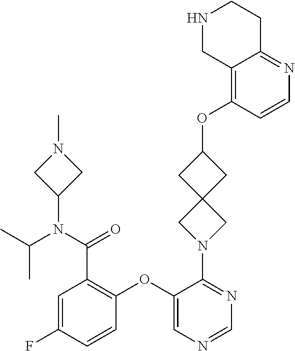 [SMILES: CC(C)N(C(=O)c1cc(F)ccc1Oc1cncnc1N1CC2(CC(Oc3ccnc4c3CNCC4)C2)C1)C1CN(C)C1] | 5-Fluoro-N-isopropyl-N-(1- methylazetidin-3-yl)-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 65 | 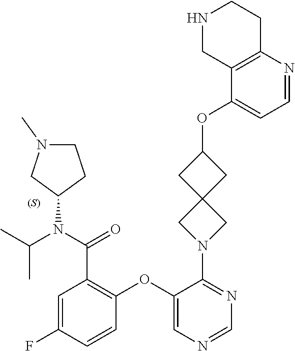 | (S)-5-Fluoro-N-isopropyl-N-(1- methylpyrrolidin-3-yl)-2-((4-(6- ((5,6,7,8-tetrahydro-1,6-naphthyridin-4- yl)oxy)-2-azaspiro[3.3]heptan-2- yl)pyrimidin-5-yl)oxy)benzamide |
| 66 | 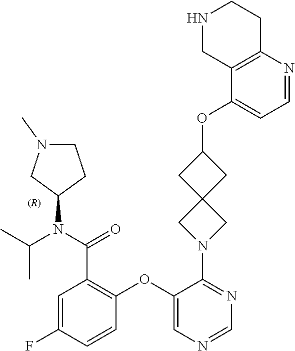 | (R)-5-Fluoro-N-isopropyl-N-(1- methylpyrrolidin-3-yl)-2-((4-(6- ((5,6,7,8-tetrahydro-1,6-naphthyridin-4- yl)oxy)-2-azaspiro[3.3]heptan-2- yl)pyrimidin-5-yl)oxy)benzamide |
| 67 | 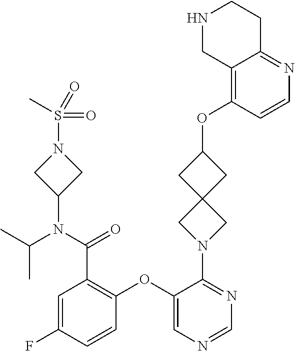 [SMILES: CC(C)N(C(=O)c1cc(F)ccc1Oc1cncnc1N1CC2(CC(Oc3ccnc4c3CNCC4)C2)C1)C1CN(S(C)(=O)=O)C1] | 5-Fluoro-N-isopropyl-N-(1- (methylsulfonyl)azetidin-3-yl)-2-((4-(6- ((5,6,7,8-tetrahydro-1,6-naphthyridin-4- yl)oxy)-2-azaspiro[3.3]heptan-2- yl)pyrimidin-5-yl)oxy)benzamide |
| 68 | 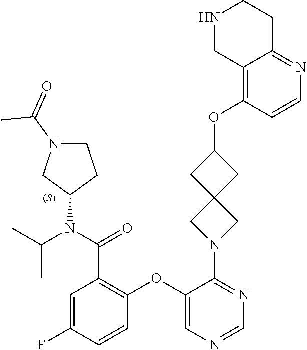 | (S)-N-(1-Acetylpyrrolidin-3-yl)-5- fluoro-N-isopropyl-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 69 | 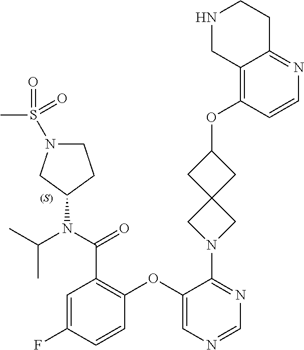 | (S)-5-Fluoro-N-isopropyl-N-(1- (methylsulfonyl)pyrrolidin-3-yl)-2-((4- (6-((5,6,7,8-tetrahydro-1,6- naphthyridin-4-yl)oxy)-2- azaspiro[3.3]heptan-2-yl)pyrimidin-5- yl)oxy)benzamide |
| 70 | 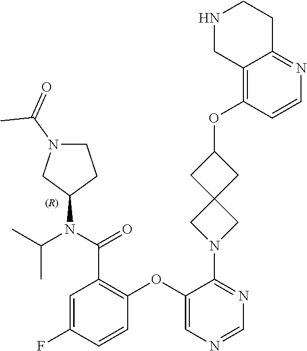 | (R)-N-(1-Acetylpyrrolidin-3-yl)-5- fluoro-N-isopropyl-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 71 | 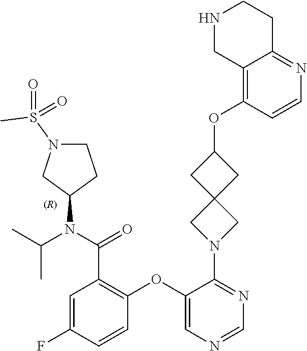 | (R)-5-Fluoro-N-isopropyl-N-(1- (methylsulfonyl)pyrrolidin-3-yl)-2-((4- (6-((5,6,7,8-tetrahydro-1,6- naphthyridin-4-yl)oxy)-2- azaspiro[3.3]heptan-2-yl)pyrimidin-5- yl)oxy)benzamide |
| 72 | 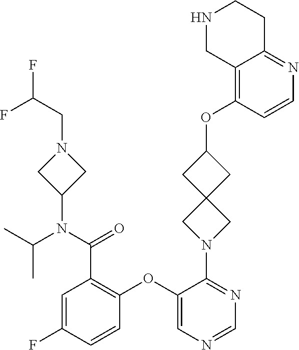 [SMILES: CC(C)N(C(=O)c1cc(F)ccc1Oc1cncnc1N1CC2(CC(Oc3ccnc4c3CNCC4)C2)C1)C1CN(CC(F)F)C1] | N-(1-(2,2-Difluoroethyl)azetidin-3-yl)- 5-fluoro-N-isopropyl-2-((4-(6-((5,6,7,8- tetrahydro-1,6-naphthyridin-4-yl)oxy)- 2-azaspiro[3.3]heptan-2-yl)pyrimidin-5- yl)oxy)benzamide |
| 73 | 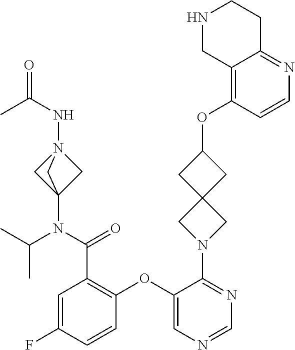 | N-(3-Acetamidobicyclo[1.1.1]pentan-1- yl)-5-fluoro-N-isopropyl-2-((4-(6- ((5,6,7,8-tetrahydro-1,6-naphthyridin-4- yl)oxy)-2-azaspiro[3.3]heptan-2- yl)pyrimidin-5-yl)oxy)benzamide |
| 74 | 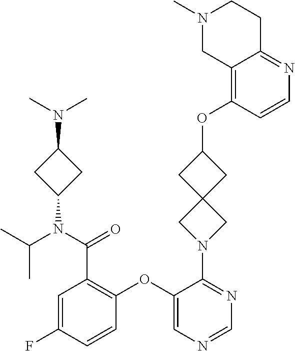 [SMILES: CC(C)N(C(=O)c1cc(F)ccc1Oc1cncnc1N1CC2(CC(Oc3ccnc4c3CN(C)CC4)C2)C1)[C@H]1C[C@H](N(C)C)C1] | N-((1r,3r)-3- (Dimethylamino)cyclobutyl)-5-fluoro- N-isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 75 | 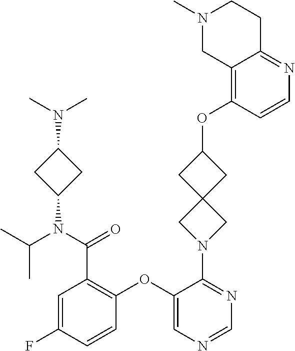 [SMILES: CC(C)N(C(=O)c1cc(F)ccc1Oc1cncnc1N1CC2(CC(Oc3ccnc4c3CN(C)CC4)C2)C1)[C@H]1C[C@@H](N(C)C)C1] | N-((1s,3s)-3- (Dimethylamino)cyclobutyl)-5-fluoro- N-isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 76 | 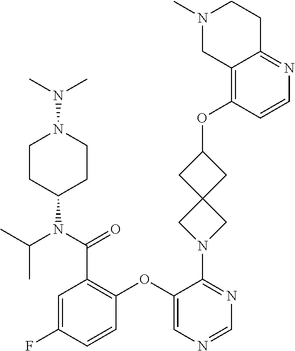 | N-((1s,4s)-4- (Dimethylamino)cyclohexyl)-5-fluoro- N-isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 77 | 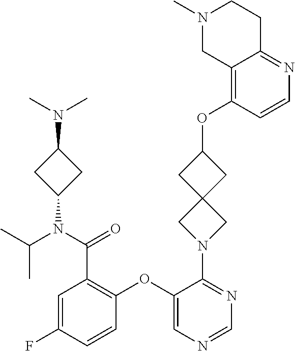 [SMILES: CC(C)N(C(=O)c1cc(F)ccc1Oc1cncnc1N1CC2(CC(Oc3ccnc4c3CN(C)CC4)C2)C1)[C@H]1C[C@H](N(C)C)C1] | N-((1r,3r)-3- (Dimethylamino)cyclobutyl)-5-fluoro-2- ((4-(6-((6-isobutyl-5,6,7,8-tetrahydro- 1,6-naphthyridin-4-yl)oxy)-2- azaspiro[3.3]heptan-2-yl)pyrimidin-5- yl)oxy)-N-isopropylbenzamide |
| 78 | 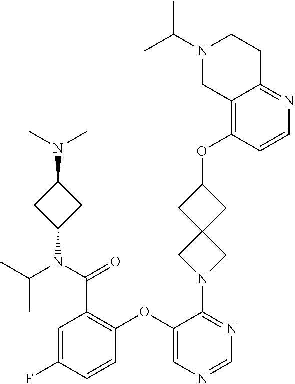 [SMILES: CC(C)N1CCc2nccc(OC3CC4(C3)CN(c3ncncc3Oc3ccc(F)cc3C(=O)N(C(C)C)[C@H]3C[C@H](N(C)C)C3)C4)c2C1] | N-((1r,3r)-3- (Dimethylamino)cyclobutyl)-5-fluoro- N-isopropyl-2-((4-(6-((6-isopropyl- 5,6,7,8-tetrahydro-1,6-naphthyridin-4- yl)oxy)-2-azaspiro[3.3]heptan-2- yl)pyrimidin-5-yl)oxy)benzamide |
| 79 | 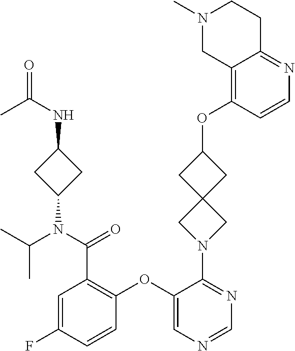 [SMILES: CC(=O)N[C@H]1C[C@H](N(C(=O)c2cc(F)ccc2Oc2cncnc2N2CC3(CC(Oc4ccnc5c4CN(C)CC5)C3)C2)C(C)C)C1] | N-((1r,3r)-3-Acetamidocyclobutyl)-5- fluoro-N-isopropyl-2-((4-(6-((6-methyl- 5,6,7,8-tetrahydro-1,6-naphthyridin-4- yl)oxy)-2-azaspiro[3.3]heptan-2- yl)pyrimidin-5-yl)oxy)benzamide |
| 80 | 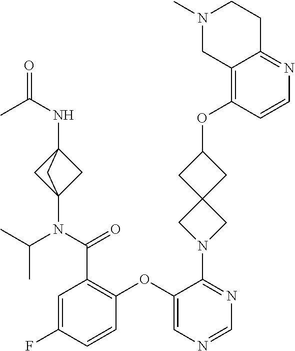 [SMILES: CC(=O)NC12CC(N(C(=O)c3cc(F)ccc3Oc3cncnc3N3CC4(CC(Oc5ccnc6c5CN(C)CC6)C4)C3)C(C)C)(C1)C2] | N-(3-Acetamidobicyclo[1.1.1]pentan-1- yl)-5-fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)benzamide |
| 81 | 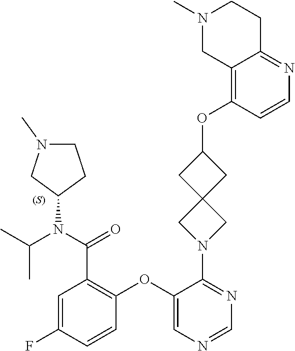 | (S)-5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-(1-methylpyrrolidin-3- yl)benzamide |
| 82 | 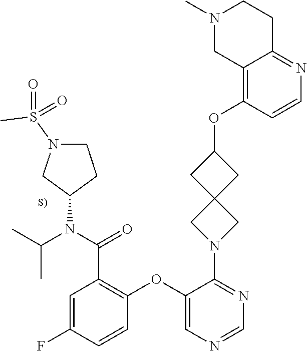 | (S)-5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-(1- (methylsulfonyl)pyrrolidin-3- yl)benzamide |
| 83 | 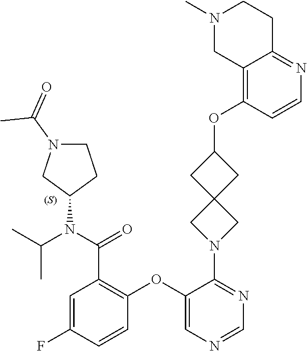 | (S)-N-(1-Acetylpyrrolidin-3-yl)-5- fluoro-N-isopropyl-2-((4-(6-((6-methyl- 5,6,7,8-tetrahydro-1,6-naphthyridin-4- yl)oxy)-2-azaspiro[3.3]heptan-2- yl)pyrimidin-5-yl)oxy)benzamide |
| 84 | 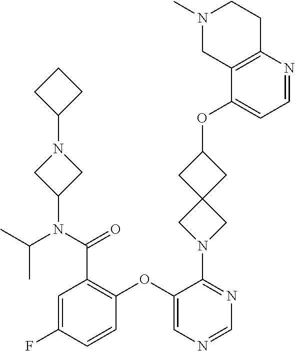 [SMILES: CC(C)N(C(=O)c1cc(F)ccc1Oc1cncnc1N1CC2(CC(Oc3ccnc4c3CN(C)CC4)C2)C1)C1CN(C2CCC2)C1] | N-(1-Cyclobutylazetidin-3-yl)-5-fluoro- N-isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 85 | 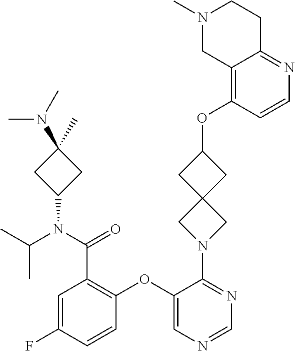 | N-((1r,3r)-3-(Dimethylamino)-3- methylcyclobutyl)-5-fluoro-N- isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 86 | 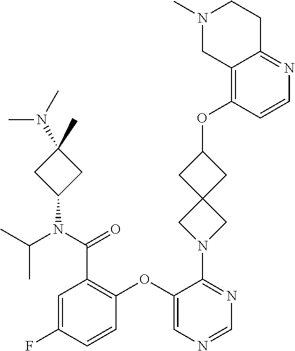 [SMILES: CC(C)N(C(=O)c1cc(F)ccc1Oc1cncnc1N1CC2(CC(Oc3ccnc4c3CN(C)CC4)C2)C1)[C@H]1C[C@@](C)(N(C)C)C1] | N-((1s,3s)-3-(Dimethylamino)-3- methylcyclobutyl)-5-fluoro-N- isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 87 | 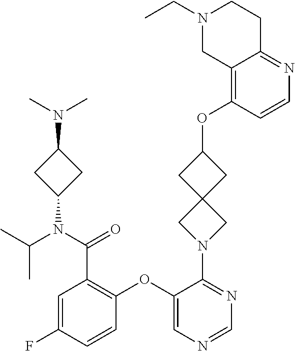 [SMILES: CCN1CCc2nccc(OC3CC4(C3)CN(c3ncncc3Oc3ccc(F)cc3C(=O)N(C(C)C)[C@H]3C[C@H](N(C)C)C3)C4)c2C1] | N-((1r,3r)-3- (Dimethylamino)cyclobutyl)-2-((4-(6- ((6-ethyl-5,6,7,8-tetrahydro-1,6- naphthyridin-4-yl)oxy)-2- azaspiro[3.3]heptan-2-yl)pyrimidin-5- yl)oxy)-5-fluoro-N-isopropylbenzamide |
| 88 | 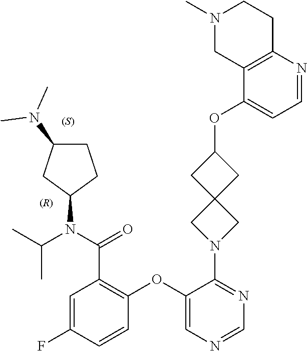 | N-((1R,3S)-3- (Dimethylamino)cyclopentyl)-5-fluoro- N-isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 89 | 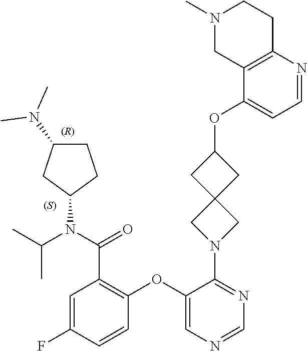 | N-((1S,3R)-3- (Dimethylamino)cyclopentyl)-5-fluoro- N-isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 90 | 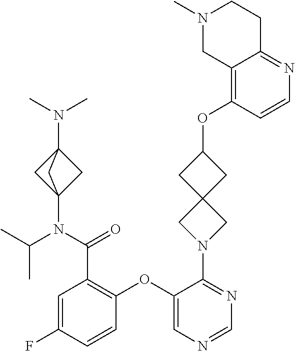 [SMILES: CC(C)N(C(=O)c1cc(F)ccc1Oc1cncnc1N1CC2(CC(Oc3ccnc4c3CN(C)CC4)C2)C1)C12CC(N(C)C)(C1)C2] | N-(3- (Dimethylamino)bicyclo[1.1.1]pentan- 1-yl)-5-fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)benzamide |
| 91 | 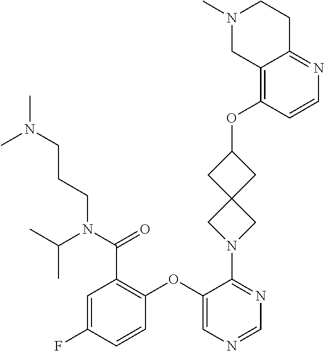 [SMILES: CC(C)N(CCCN(C)C)C(=O)c1cc(F)ccc1Oc1cncnc1N1CC2(CC(Oc3ccnc4c3CN(C)CC4)C2)C1] | N-(3-(Dimethylamino)propyl)-5-fluoro- N-isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 92 | 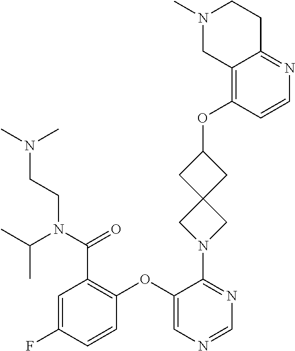 [SMILES: CC(C)N(CCN(C)C)C(=O)c1cc(F)ccc1Oc1cncnc1N1CC2(CC(Oc3ccnc4c3CN(C)CC4)C2)C1] | N-(2-(Dimethylamino)ethyl)-5-fluoro- N-isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 93 | 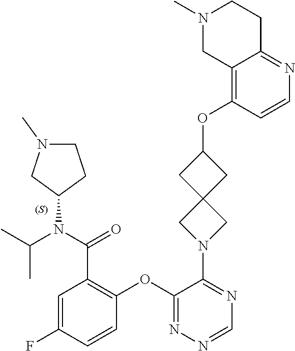 | (S)-5-Fluoro-N-isopropyl-2-((5-(6-((6- methyl-5,6,7,8-tetrahydro-1,6- naphthyridin-4-yl)oxy)-2- azaspiro[3.3]heptan-2-yl)-1,2,4-triazin- 6-yl)oxy)-N-(1-methylpyrrolidin-3- yl)benzamide |
| 94 | 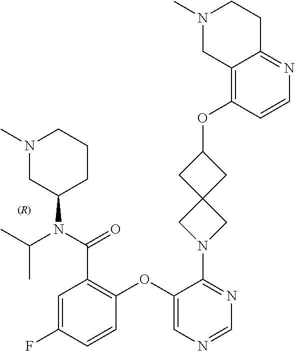 | (R)-5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-(1-methylpiperidin-3- yl)benzamide |
| 95 | 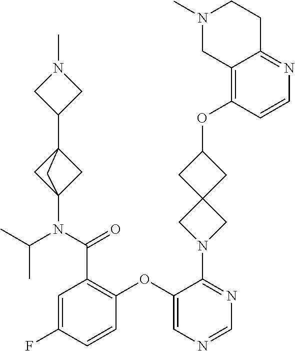 [SMILES: CC(C)N(C(=O)c1cc(F)ccc1Oc1cncnc1N1CC2(CC(Oc3ccnc4c3CN(C)CC4)C2)C1)C12CC(C3CN(C)C3)(C1)C2] | (D1)-5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-(3-(1-methylazetidin-3- yl)cyclobutyl)benzamide |
| | (D1) | |
| 96 | 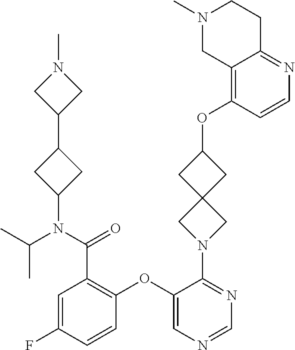 [SMILES: CC(C)N(C(=O)c1cc(F)ccc1Oc1cncnc1N1CC2(CC(Oc3ccnc4c3CN(C)CC4)C2)C1)C1CC(C2CN(C)C2)C1] | (D2)-5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-(3-(1-methylazetidin-3- yl)cyclobutyl)benzamide |
| | (D2) | |
| 97 | 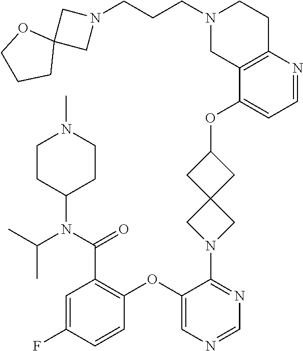 [SMILES: CC(C)N(C(=O)c1cc(F)ccc1Oc1cncnc1N1CC2(CC(Oc3ccnc4c3CN(CCCN3CC5(CCCO5)C3)CC4)C2)C1)C1CCN(C)CC1] | 2-((4-(6-((6-(3-(5-Oxa-2- azaspiro[3.4]octan-2-yl)propyl)-5,6,7,8- tetrahydro-1,6-naphthyridin-4-yl)oxy)- 2-azaspiro[3.3]heptan-2-yl)pyrimidin-5- yl)oxy)-5-fluoro-N-isopropyl-N-(1- methylpiperidin-4-yl)benzamide |
| 98 | 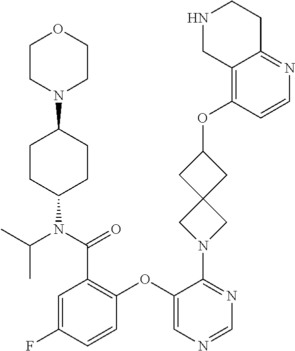 [SMILES: CC(C)N(C(=O)c1cc(F)ccc1Oc1cncnc1N1CC2(CC(Oc3ccnc4c3CNCC4)C2)C1)[C@H]1CC[C@H](N2CCOCC2)CC1] | 5-Fluoro-N-isopropyl-N-((1r,3r)-3- morpholinocyclobutyl)-2-((4-(6- ((5,6,7,8-tetrahydro-1,6-naphthyridin-4- yl)oxy)-2-azaspiro[3.3]heptan-2- yl)pyrimidin-5-yl)oxy)benzamide |
| 99 | 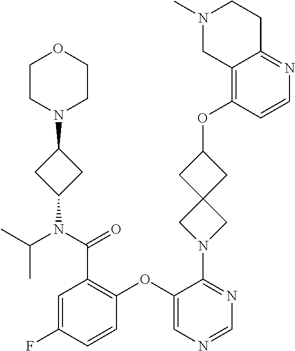 [SMILES: CC(C)N(C(=O)c1cc(F)ccc1Oc1cncnc1N1CC2(CC(Oc3ccnc4c3CN(C)CC4)C2)C1)[C@H]1C[C@H](N2CCOCC2)C1] | 5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-((1r,3r)-3- morpholinocyclobutyl)benzamide |
| 100 | 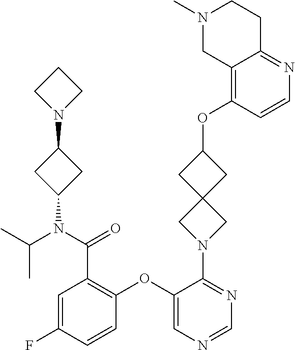 [SMILES: CC(C)N(C(=O)c1cc(F)ccc1Oc1cncnc1N1CC2(CC(Oc3ccnc4c3CN(C)CC4)C2)C1)[C@H]1C[C@H](N2CCC2)C1] | N-((1r,3r)-3-(Azetidin-1-yl)cyclobutyl)- 5-fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)benzamide |
| 101 | 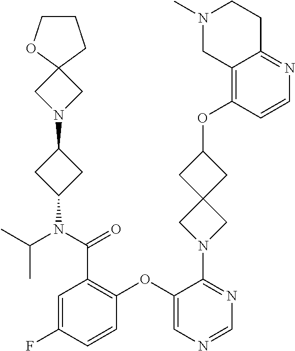 [SMILES: CC(C)N(C(=O)c1cc(F)ccc1Oc1cncnc1N1CC2(CC(Oc3ccnc4c3CN(C)CC4)C2)C1)[C@H]1C[C@H](N2CC3(CCCO3)C2)C1] | N-((1r,3r)-3-(5-Oxa-2- azaspiro[3.4]octan-2-yl)cyclobutyl)-5- fluoro-N-isopropyl-2-((4-(6-((6-methyl- 5,6,7,8-tetrahydro-1,6-naphthyridin-4- yl)oxy)-2-azaspiro[3.3]heptan-2- yl)pyrimidin-5-yl)oxy)benzamide |
| 102 | 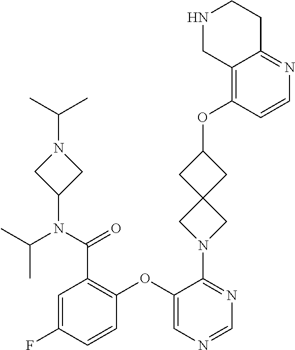 [SMILES: CC(C)N1CC(N(C(=O)c2cc(F)ccc2Oc2cncnc2N2CC3(CC(Oc4ccnc5c4CNCC5)C3)C2)C(C)C)C1] | 5-Fluoro-N-isopropyl-N-(1- isopropylazetidin-3-yl)-2-((4-(6- ((5,6,7,8-tetrahydro-1,6-naphthyridin-4- yl)oxy)-2-azaspiro[3.3]heptan-2- yl)pyrimidin-5-yl)oxy)benzamide |
| 103 | 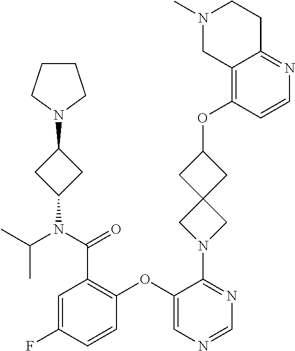 [SMILES: CC(C)N(C(=O)c1cc(F)ccc1Oc1cncnc1N1CC2(CC(Oc3ccnc4c3CN(C)CC4)C2)C1)[C@H]1C[C@H](N2CCCC2)C1] | 5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-((1r,3r)-3-(pyrrolidin-1- yl)cyclobutyl)benzamide |
| 104 | 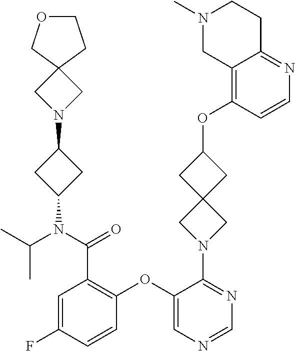 [SMILES: CC(C)N(C(=O)c1cc(F)ccc1Oc1cncnc1N1CC2(CC(Oc3ccnc4c3CN(C)CC4)C2)C1)[C@H]1C[C@H](N2CC3(CCOC3)C2)C1] | N-((1r,3r)-3-(6-Oxa-2- azaspiro[3.4]octan-2-yl)cyclobutyl)-5- fluoro-N-isopropyl-2-((4-(6-((6-methyl- 5,6,7,8-tetrahydro-1,6-naphthyridin-4- yl)oxy)-2-azaspiro[3.3]heptan-2- yl)pyrimidin-5-yl)oxy)benzamide |
| 105 | 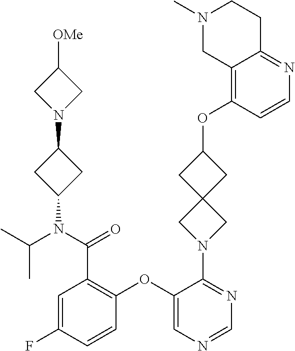 [SMILES: COC1CN([C@H]2C[C@H](N(C(=O)c3cc(F)ccc3Oc3cncnc3N3CC4(CC(Oc5ccnc6c5CN(C)CC6)C4)C3)C(C)C)C2)C1] | 5-Fluoro-N-isopropyl-N-((1r,3r)-3-(3- methoxyazetidin-1-yl)cyclobutyl)-2-((4- (6-((6-methyl-5,6,7,8-tetrahydro-1,6- naphthyridin-4-yl)oxy)-2- azaspiro[3.3]heptan-2-yl)pyrimidin-5- yl)oxy)benzamide |
| 106 | 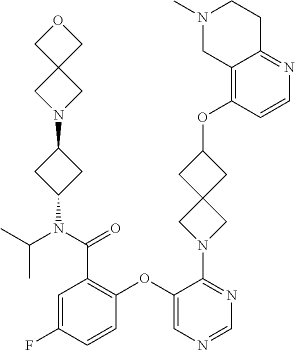 [SMILES: CC(C)N(C(=O)c1cc(F)ccc1Oc1cncnc1N1CC2(CC(Oc3ccnc4c3CN(C)CC4)C2)C1)[C@H]1C[C@H](N2CC3(COC3)C2)C1] | N-((1r,3r)-3-(2-Oxa-6- azaspiro[3.3]heptan-6-yl)cyclobutyl)-5- fluoro-N-isopropyl-2-((4-(6-((6-methyl- 5,6,7,8-tetrahydro-1,6-naphthyridin-4- yl)oxy)-2-azaspiro[3.3]heptan-2- yl)pyrimidin-5-yl)oxy)benzamide |
| 107 | 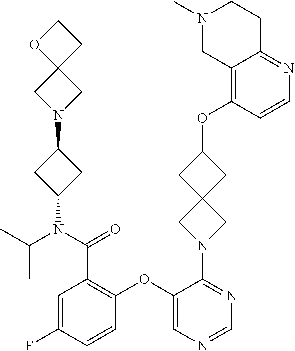 [SMILES: CC(C)N(C(=O)c1cc(F)ccc1Oc1cncnc1N1CC2(CC(Oc3ccnc4c3CN(C)CC4)C2)C1)[C@H]1C[C@H](N2CC3(CCO3)C2)C1] | N-((1r,3r)-3-(1-Oxa-6- azaspiro[3.3]heptan-6-yl)cyclobutyl)-5- fluoro-N-isopropyl-2-((4-(6-((6-methyl- 5,6,7,8-tetrahydro-1,6-naphthyridin-4- yl)oxy)-2-azaspiro[3.3]heptan-2- yl)pyrimidin-5-yl)oxy)benzamide |
| 108 | 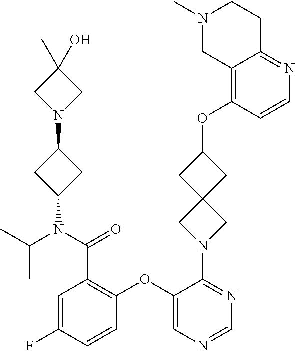 [SMILES: CC(C)N(C(=O)c1cc(F)ccc1Oc1cncnc1N1CC2(CC(Oc3ccnc4c3CN(C)CC4)C2)C1)[C@H]1C[C@H](N2CC(C)(O)C2)C1] | 5-Fluoro-N-((1r,3r)-3-(3-hydroxy-3- methylazetidin-1-yl)cyclobutyl)-N- isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 109 | 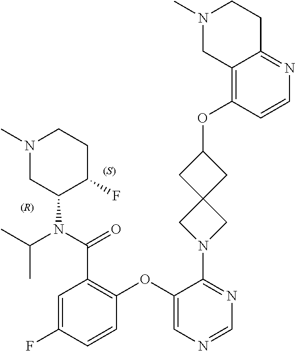 | 5-Fluoro-N-((3R,4S)-4-fluoro-1- methylpiperidin-3-yl)-N-isopropyl-2- ((4-(6-((6-methyl-5,6,7,8-tetrahydro- 1,6-naphthyridin-4-yl)oxy)-2- azaspiro[3.3]heptan-2-yl)pyrimidin-5- yl)oxy)benzamide |
| 110 | 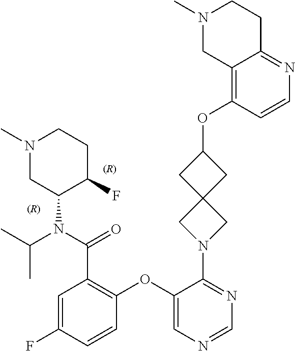 | 5-Fluoro-N-((3R,4R)-4-fluoro-1- methylpiperidin-3-yl)-N-isopropyl-2- ((4-(6-((6-methyl-5,6,7,8-tetrahydro- 1,6-naphthyridin-4-yl)oxy)-2- azaspiro[3.3]heptan-2-yl)pyrimidin-5- yl)oxy)benzamide |
| 111 | 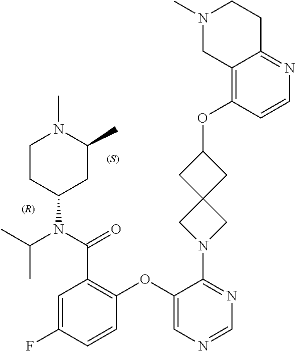 | N-((2S,4R)-1,2-Dimethylpiperidin-4-yl)- 5-fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)benzamide |
| 112 | 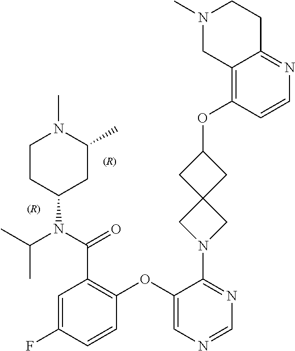 | N-((2R,4R)-1,2-Dimethylpiperidin-4- yl)-5-fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)benzamide |
| 113 | 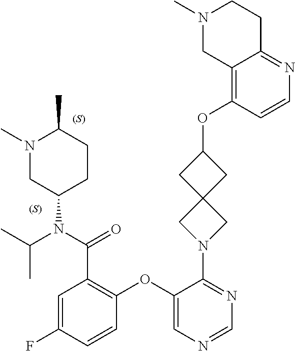 | N-((3S,6S)-1,6-Dimethylpiperidin-3-yl)- 5-fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)benzamide |
| 114 | 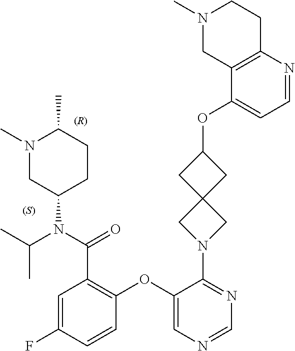 | N-((3S,6R)-1,6-Dimethylpiperidin-3-yl)- 5-fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)benzamide |
| 115 | 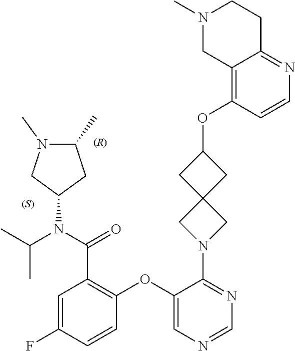 | N-((3S,5R)-1,5-Dimethylpyrrolidin-3- yl)-5-fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)benzamide |
| 116 | 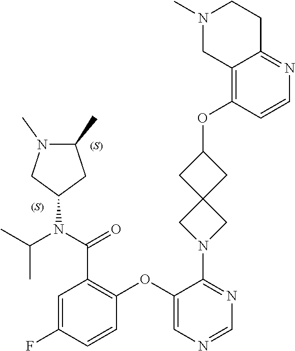 | N-((3S,5S)-1,5-Dimethylpyrrolidin-3- yl)-5-fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)benzamide |
| 117 | 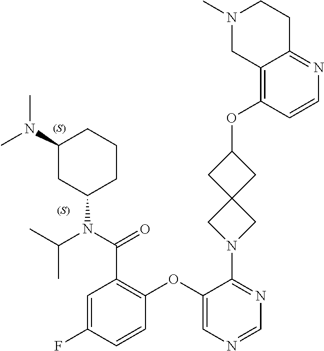 | N-((1S,3S)-3- (Dimethylamino)cyclohexyl)-5-fluoro- N-isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 118 | 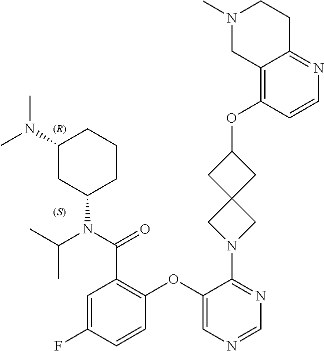 | N-((1S,3R)-3- (Dimethylamino)cyclohexyl)-5-fluoro- N-isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 119 | 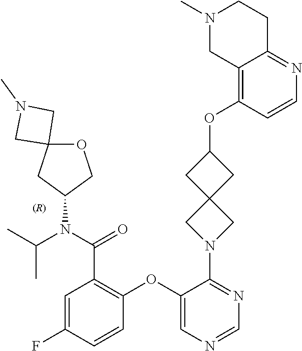 | (R)-5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-(2-methyl-5-oxa-2- azaspiro[3.4]octan-7-yl)benzamide |
| 120 | 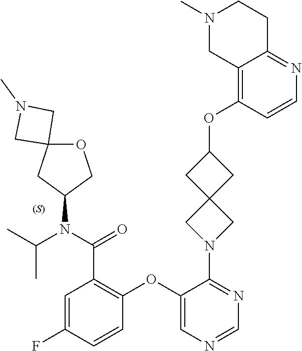 | (S)-5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-(2-methyl-5-oxa-2- azaspiro[3.4]octan-7-yl)benzamide |
| 121 | 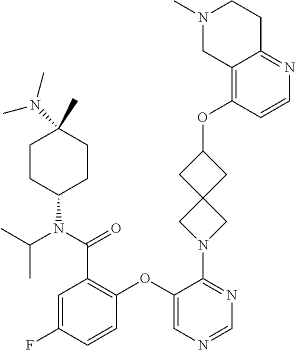 [SMILES: CC(C)N(C(=O)c1cc(F)ccc1Oc1cncnc1N1CC2(CC(Oc3ccnc4c3CN(C)CC4)C2)C1)[C@H]1CC[C@@](C)(N(C)C)CC1] | N-((1s,4s)-4-(Dimethylamino)-4- methylcyclohexyl)-5-fluoro-N- isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 122 | 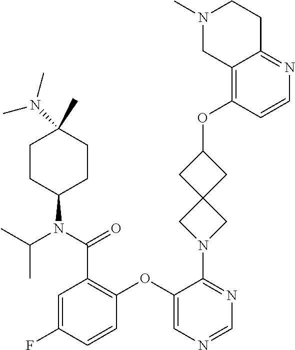 [SMILES: CC(C)N(C(=O)c1cc(F)ccc1Oc1cncnc1N1CC2(CC(Oc3ccnc4c3CN(C)CC4)C2)C1)[C@H]1CC[C@](C)(N(C)C)CC1] | N-((1r,4r)-4-(Dimethylamino)-4- methylcyclohexyl)-5-fluoro-N- isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 123 | 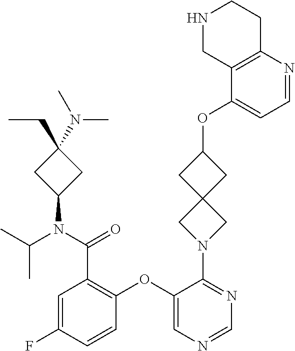 [SMILES: CC[C@]1(N(C)C)C[C@H](N(C(=O)c2cc(F)ccc2Oc2cncnc2N2CC3(CC(Oc4ccnc5c4CNCC5)C3)C2)C(C)C)C1] | N-((1s,3r)-3-(Dimethylamino)-3- ethylcyclobutyl)-5-fluoro-N-isopropyl- 2-((4-(6-((5,6,7,8-tetrahydro-1,6- naphthyridin-4-yl)oxy)-2- azaspiro[3.3]heptan-2-yl)pyrimidin-5- yl)oxy)benzamide |
| 124 | 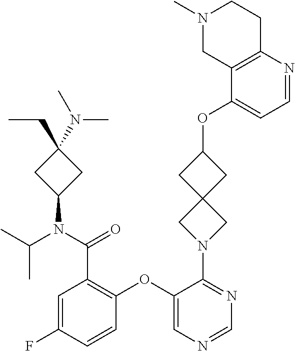 [SMILES: CC[C@]1(N(C)C)C[C@H](N(C(=O)c2cc(F)ccc2Oc2cncnc2N2CC3(CC(Oc4ccnc5c4CN(C)CC5)C3)C2)C(C)C)C1] | N-((1s,3r)-3-(Dimethylamino)-3- ethylcyclobutyl)-5-fluoro-N-isopropyl- 2-((4-(6-((6-methyl-5,6,7,8-tetrahydro- 1,6-naphthyridin-4-yl)oxy)-2- azaspiro[3.3]heptan-2-yl)pyrimidin-5- yl)oxy)benzamide |
| 125 | 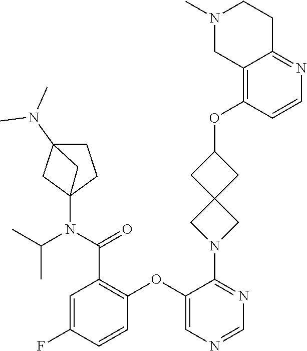 [SMILES: CC(C)N(C(=O)c1cc(F)ccc1Oc1cncnc1N1CC2(CC(Oc3ccnc4c3CN(C)CC4)C2)C1)C12CCC(N(C)C)(C1)C2] | N-(4- (Dimethylamino)bicyclo[2.1.1]hexan-1- yl)-5-fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)benzamide |
| 126 | 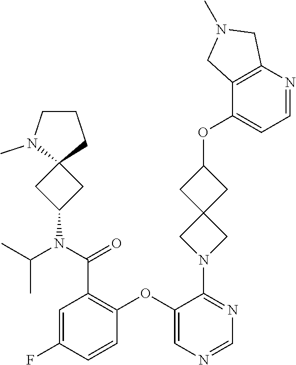 [SMILES: CC(C)N(C(=O)c1cc(F)ccc1Oc1cncnc1N1CC2(CC(Oc3ccnc4c3CN(C)C4)C2)C1)[C@H]1C[C@]2(CCCN2C)C1] | 5-Fluoro-N-isopropyl-2-((4-(6-((6- methyl-6,7-dihydro-5H-pyrrolo[3,4- b]pyridin-4-yl)oxy)-2- azaspiro[3.3]heptan-2-yl)pyrimidin-5- yl)oxy)-N-((2s,4r)-5-methyl-5- azaspiro[3.4]octan-2-yl)benzamide |
| 127 | 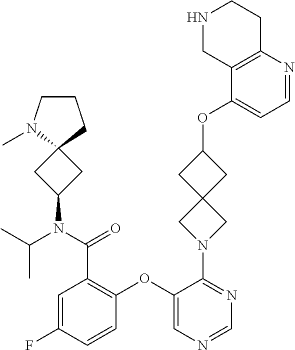 [SMILES: CC(C)N(C(=O)c1cc(F)ccc1Oc1cncnc1N1CC2(CC(Oc3ccnc4c3CNCC4)C2)C1)[C@H]1C[C@@]2(CCCN2C)C1] | 5-Fluoro-N-isopropyl-N-((2s,4r)-5- methyl-5-azaspiro[3.4]octan-2-yl)-2-((4- (6-((5,6,7,8-tetrahydro-1,6- naphthyridin-4-yl)oxy)-2- azaspiro[3.3]heptan-2-yl)pyrimidin-5- yl)oxy)benzamide |
| 128 | 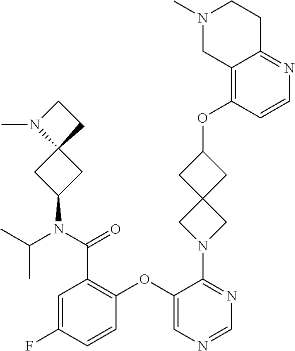 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCN2C)C1] | 5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-((4r,6s)-1-methyl-1- azaspiro[3.3]heptan-6-yl)benzamide |
| 129 | 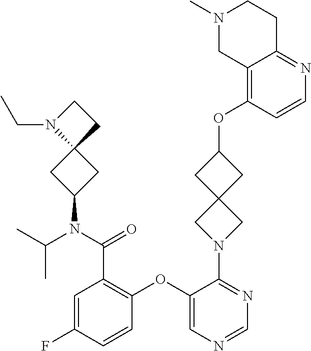 [SMILES: CCN1CC[C@]12C[C@@H](N(C(=O)c1cc(F)ccc1Oc1cncnc1N1CC3(CC(Oc4ccnc5c4CN(C)CC5)C3)C1)C(C)C)C2] | N-((4r,6s)-1-Ethyl-1- azaspiro[3.3]heptan-6-yl)-5-fluoro-N- isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 130 | 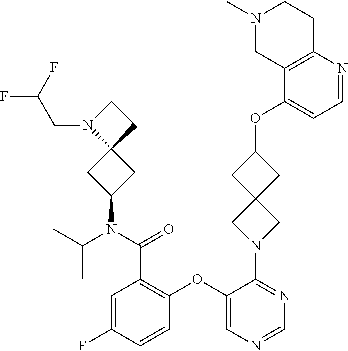 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCN2CC(F)F)C1] | N-((4r,6s)-1-(2,2-Difluoroethyl)-1- azaspiro[3.3]heptan-6-yl)-5-fluoro-N- isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 131 | 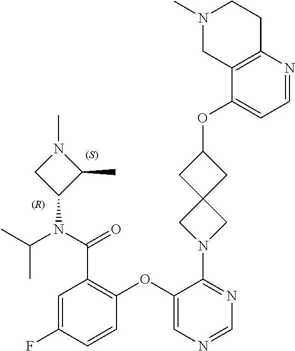 | N-((2S,3R)-1,2-Dimethylazetidin-3-yl)- 5-fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)benzamide |
| 132 | 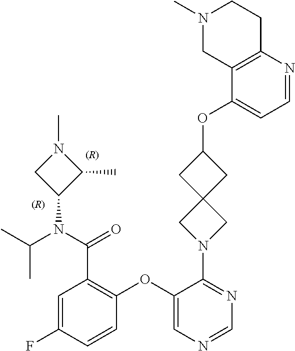 | N-((2R,3R)-1,2-dimethylazetidin-3-yl)- 5-fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)benzamide |
| 133 | 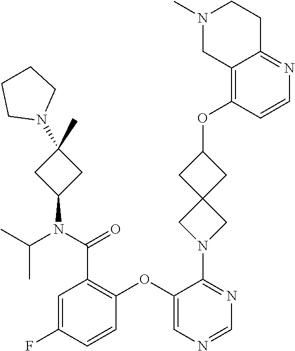 [SMILES: CC(C)N(C(=O)c1cc(F)ccc1Oc1cncnc1N1CC2(CC(Oc3ccnc4c3CN(C)CC4)C2)C1)[C@H]1C[C@](C)(N2CCCC2)C1] | 5-Fluoro-N-isopropyl-N-((1r,3r)-3- methyl-3-(pyrrolidin-1-yl)cyclobutyl)-2- ((4-(6-((6-methyl-5,6,7,8-tetrahydro- 1,6-naphthyridin-4-yl)oxy)-2- azaspiro[3.3]heptan-2-yl)pyrimidin-5- yl)oxy)benzamide |
| 134 | 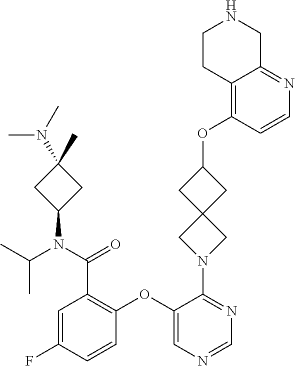 [SMILES: CC(C)N(C(=O)c1cc(F)ccc1Oc1cncnc1N1CC2(CC(Oc3ccnc4c3CCNC4)C2)C1)[C@H]1C[C@](C)(N(C)C)C1] | N-((1r,3r)-3-(Dimethylamino)-3- methylcyclobutyl)-5-fluoro-N- isopropyl-2-((4-(6-((5,6,7,8-tetrahydro- 1,7-naphthyridin-4-yl)oxy)-2- azaspiro[3.3]heptan-2-yl)pyrimidin-5- yl)oxy)benzamide |
| 135 | 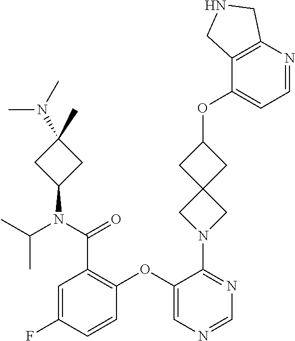 [SMILES: CC(C)N(C(=O)c1cc(F)ccc1Oc1cncnc1N1CC2(CC(Oc3ccnc4c3CNC4)C2)C1)[C@H]1C[C@](C)(N(C)C)C1] | 2-((4-(6-((6,7-Dihydro-5H-pyrrolo[3,4- b]pyridin-4-yl)oxy)-2- azaspiro[3.3]heptan-2-yl)pyrimidin-5- yl)oxy)-N-((1r,3r)-3-(dimethylamino)- 3-methylcyclobutyl)-5-fluoro-N- isopropylbenzamide |
| 136 | 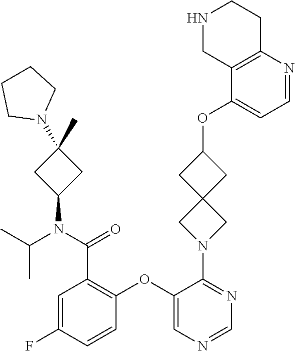 [SMILES: CC(C)N(C(=O)c1cc(F)ccc1Oc1cncnc1N1CC2(CC(Oc3ccnc4c3CNCC4)C2)C1)[C@H]1C[C@](C)(N2CCCC2)C1] | 5-Fluoro-N-isopropyl-N-((1r,3r)-3- methyl-3-(pyrrolidin-1-yl)cyclobutyl)-2- ((4-(6-((5,6,7,8-tetrahydro-1,6- naphthyridin-4-yl)oxy)-2- azaspiro[3.3]heptan-2-yl)pyrimidin-5- yl)oxy)benzamide |
| 137 | 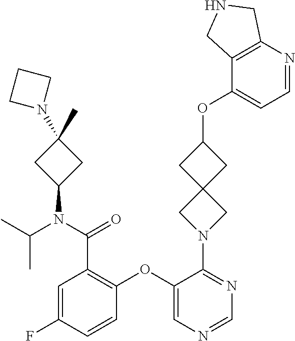 [SMILES: CC(C)N(C(=O)c1cc(F)ccc1Oc1cncnc1N1CC2(CC(Oc3ccnc4c3CNC4)C2)C1)[C@H]1C[C@](C)(N2CCC2)C1] | N-((1r,3r)-3-(Azetidin-1-yl)-3- methylcyclobutyl)-2-((4-(6-((6,7- dihydro-5H-pyrrolo[3,4-b]pyridin-4- yl)oxy)-2-azaspiro[3.3]heptan-2- yl)pyrimidin-5-yl)oxy)-5-fluoro-N- isopropylbenzamide |
| 138 | 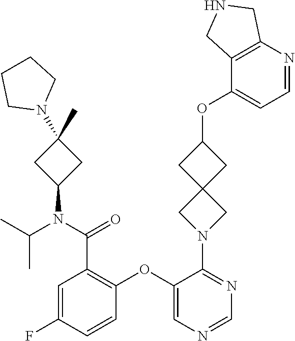 [SMILES: CC(C)N(C(=O)c1cc(F)ccc1Oc1cncnc1N1CC2(CC(Oc3ccnc4c3CNC4)C2)C1)[C@H]1C[C@](C)(N2CCCC2)C1] | 2-((4-(6-((6,7-Dihydro-5H-pyrrolo[3,4- b]pyridin-4-yl)oxy)-2-azaspiro[3.3] heptan-2-yl)pyrimidin-5-yl)oxy)-5- fluoro-N-isopropyl-N-((1r,3r)-3-methyl- 3-(pyrrolidin-1-yl)cyclobutyl) benzamide |
| 139 | 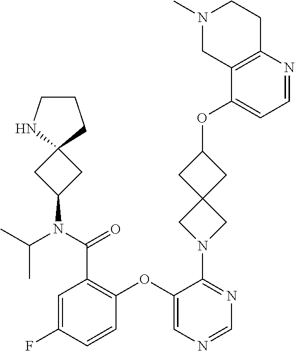 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCN2)C1] | 5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-((2s,4r)-5-azaspiro[3.4]octan- 2-yl)benzamide |
| 140 | 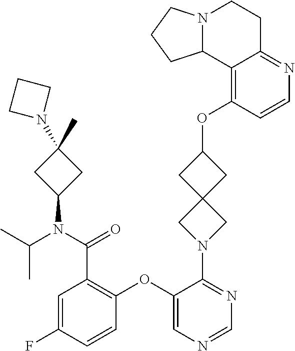 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(N2CCC2)C1] | (D1)-N-((1r,3r)-3-(Azetidin-1-yl)-3- methylcyclobutyl)-5-fluoro-2-((4-(6- ((5,6,8,9,10,10a-hexahydropyrrolo[2,1- f][1,6]naphthyridin-1-yl)oxy)-2- azaspiro[3.3]heptan-2-yl)pyrimidin-5- yl)oxy)-N-isopropylbenzamide |
| | (D1) | |
| 141 | 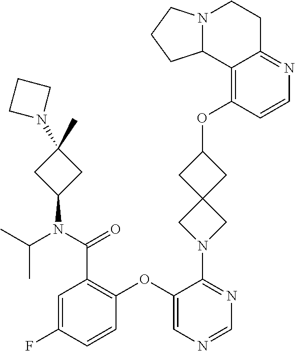 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(N2CCC2)C1] | (D2)-N-((1r,3r)-3-(Azetidin-1-yl)-3- methylcyclobutyl)-5-fluoro-2-((4-(6- ((5,6,8,9,10,10a-hexahydropyrrolo[2,1- f][1,6]naphthyridin-1-yl)oxy)-2- azaspiro[3.3]heptan-2-yl)pyrimidin-5- yl)oxy)-N-isopropylbenzamide |
| | (D2) | |
| 142 | 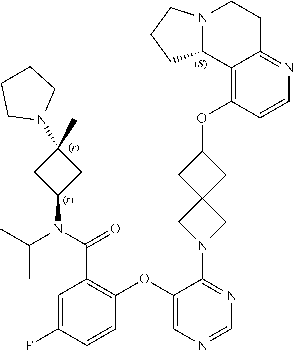 | 5-Fluoro-2-((4-(6-(((S)-5,6,8,9,10,10a- hexahydropyrrolo[2,1-f] [1,6]naphthyridin-1-yl)oxy)-2- azaspiro[3.3]heptan-2-yl)pyrimidin-5- yl)oxy)-N-isopropyl-N-((1r,3r)-3- methyl-3-(pyrrolidin-1- yl)cyclobutyl)benzamide or (S)-5-Fluoro-2-((4-(6-((5,6,8,9,10,10a- hexahydropyrrolo[2,1-f] [1,6]naphthyridin-1-yl)oxy)-2- azaspiro[3.3]heptan-2-yl)pyrimidin-5- yl)oxy)-N-isopropyl-N-((1r,3r)-3- methyl-3-(pyrrolidin-1- yl)cyclobutyl)benzamide |
| 143 | 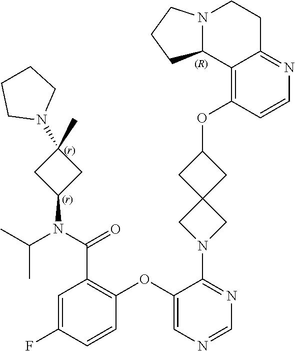 | 5-Fluoro-2-((4-(6-(((R)-5,6,8,9,10,10a- hexahydropyrrolo[2,1-f] [1,6]naphthyridin-1-yl)oxy)-2- azaspiro[3.3]heptan-2-yl)pyrimidin-5- yl)oxy)-N-isopropyl-N-((1r,3r)-3- methyl-3-(pyrrolidin-1- yl)cyclobutyl)benzamide or (R)-5-Fluoro-2-((4-(6-((5,6,8,9,10,10a- hexahydropyrrolo[2,1-f] [1,6]naphthyridin-1-yl)oxy)-2- azaspiro[3.3]heptan-2-yl)pyrimidin-5- yl)oxy)-N-isopropyl-N-((1r,3r)-3- methyl-3-(pyrrolidin-1- yl)cyclobutyl)benzamide |
| 144 | 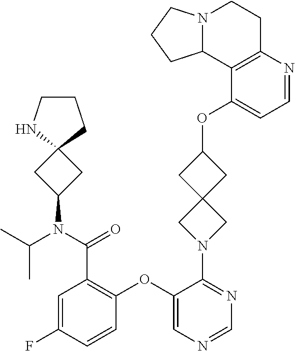 [SMILES: CC(C)N(C(=O)c1cc(F)ccc1Oc1cncnc1N1CC2(CC(Oc3ccnc4c3C3CCCN3CC4)C2)C1)[C@H]1C[C@@]2(CCCN2)C1] | (D1)-5-Fluoro-2-((4-(6-((5,6,8,9,10,10a- hexahydropyrrolo[2,1- [f][1,6]naphthyridin-1-yl)oxy)-2- azaspiro[3.3]heptan-2-yl)pyrimidin-5- yl)oxy)-N-isopropyl-N-((2s,4r)-5- azaspiro[3.4]octan-2-yl)benzamide |
| | (D1) | |
| 145 | 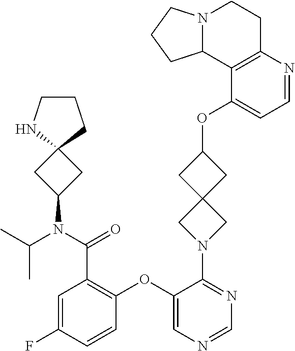 [SMILES: CC(C)N(C(=O)c1cc(F)ccc1Oc1cncnc1N1CC2(CC(Oc3ccnc4c3C3CCCN3CC4)C2)C1)[C@H]1C[C@@]2(CCCN2)C1] | (D2)-5-Fluoro-2-((4-(6-((5,6,8,9,10,10a- hexahydropyrrolo[2,1- f][1,6]naphthyridin-1-yl)oxy)-2- azaspiro[3.3]heptan-2-yl)pyrimidin-5- yl)oxy)-N-isopropyl-N-((2s,4r)-5- azaspiro[3.4]octan-2-yl)benzamide |
| | (D2) | |
| 146 | 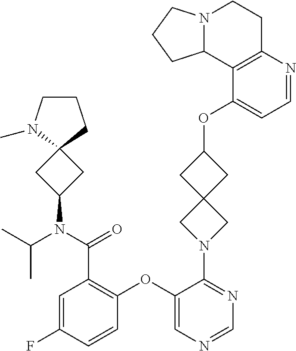 [SMILES: CC(C)N(C(=O)c1cc(F)ccc1Oc1cncnc1N1CC2(CC(Oc3ccnc4c3C3CCCN3CC4)C2)C1)[C@H]1C[C@@]2(CCCN2C)C1] | (D1)-5-Fluoro-2-((4-(6-((5,6,8,9,10,10a- hexahydropyrrolo[2,1- f][1,6]naphthyridin-1-yl)oxy)-2- azaspiro[3.3]heptan-2-yl)pyrimidin-5- yl)oxy)-N-isopropyl-N-((2s,4r)-5- methyl-5-azaspiro[3.4]octan-2- yl)benzamide |
| | (D1) | |
| 147 | 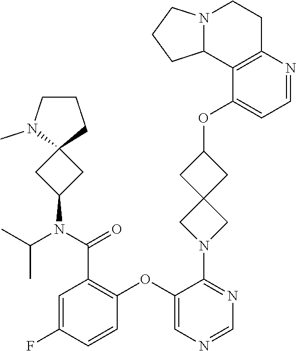 [SMILES: CC(C)N(C(=O)c1cc(F)ccc1Oc1cncnc1N1CC2(CC(Oc3ccnc4c3C3CCCN3CC4)C2)C1)[C@H]1C[C@@]2(CCCN2C)C1] | (D2)-5-Fluoro-2-((4-(6-((5,6,8,9,10,10a- hexahydropyrrolo[2,1- f][1,6]naphthyridin-1-yl)oxy)-2- azaspiro[3.3]heptan-2-yl)pyrimidin-5- yl)oxy)-N-isopropyl-N-((2s,4r)-5- methyl-5-azaspiro[3.4]octan-2- yl)benzamide |
| | (D2) | |
| 148 | 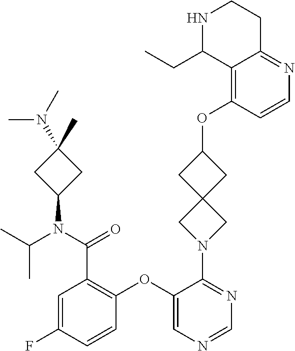 [SMILES: CCC1NCCc2nccc(OC3CC4(C3)CN(c3ncncc3Oc3ccc(F)cc3C(=O)N(C(C)C)[C@H]3C[C@](C)(N(C)C)C3)C4)c21] | (D1)-N-((1r,3r)-3-(Dimethylamino)-3- methylcyclobutyl)-2-((4-(6-((5-ethyl- 5,6,7,8-tetrahydro-1,6-naphthyridin-4- yl)oxy)-2-azaspiro[3.3]heptan-2- yl)pyrimidin-5-yl)oxy)-5-fluoro-N- isopropylbenzamide |
| | (D1) | |
| 149 | 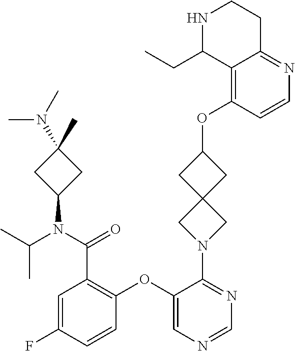 [SMILES: CCC1NCCc2nccc(OC3CC4(C3)CN(c3ncncc3Oc3ccc(F)cc3C(=O)N(C(C)C)[C@H]3C[C@](C)(N(C)C)C3)C4)c21] | (D2)-N-((1r,3r)-3-(Dimethylamino)-3- methylcyclobutyl)-2-((4-(6-((5-ethyl- 5,6,7,8-tetrahydro-1,6-naphthyridin-4- yl)oxy)-2-azaspiro[3.3]heptan-2- yl)pyrimidin-5-yl)oxy)-5-fluoro-N- isopropylbenzamide |
| | (D2) | |
| 150 | 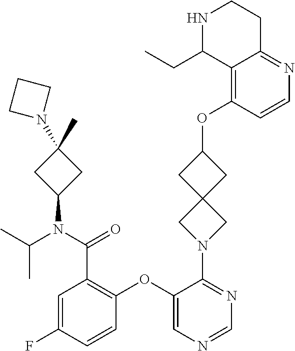 [SMILES: CCC1NCCc2nccc(OC3CC4(C3)CN(c3ncncc3Oc3ccc(F)cc3C(=O)N(C(C)C)[C@H]3C[C@](C)(N5CCC5)C3)C4)c21] | (D1)-N-((1r,3r)-3-(Azetidin-1-yl)-3- methylcyclobutyl)-2-((4-(6-((5-ethyl- 5,6,7,8-tetrahydro-1,6-naphthyridin-4- yl)oxy)-2-azaspiro[3.3]heptan-2- yl)pyrimidin-5-yl)oxy)-5-fluoro-N- isopropylbenzamide |
| | (D1) | |
| 151 | 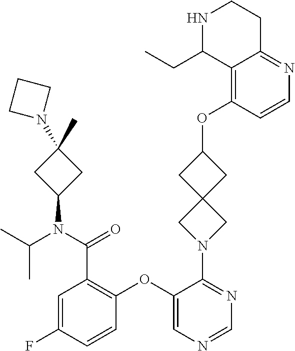 [SMILES: CCC1NCCc2nccc(OC3CC4(C3)CN(c3ncncc3Oc3ccc(F)cc3C(=O)N(C(C)C)[C@H]3C[C@](C)(N5CCC5)C3)C4)c21] | (D2)-N-((1r,3r)-3-(Azetidin-1-yl)-3- methylcyclobutyl)-2-((4-(6-((5-ethyl- 5,6,7,8-tetrahydro-1,6-naphthyridin-4- yl)oxy)-2-azaspiro[3.3]heptan-2- yl)pyrimidin-5-yl)oxy)-5-fluoro-N- isopropylbenzamide |
| | (D2) | |
| 152 | 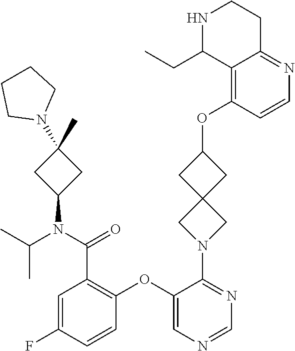 [SMILES: CCC1NCCc2nccc(OC3CC4(C3)CN(c3ncncc3Oc3ccc(F)cc3C(=O)N(C(C)C)[C@H]3C[C@](C)(N5CCCC5)C3)C4)c21] | (D1)-2-((4-(6-((5-Ethyl-5,6,7,8- tetrahydro-1,6-naphthyridin-4-yl)oxy)- 2-azaspiro[3.3]heptan-2-yl)pyrimidin-5- yl)oxy)-5-fluoro-N-isopropyl-N- ((1r,3r)-3-methyl-3-(pyrrolidin-1- yl)cyclobutyl)benzamide |
| | (D1) | |
| 153 | 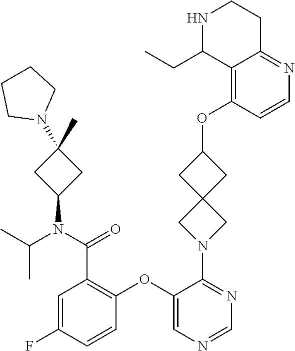 [SMILES: CCC1NCCc2nccc(OC3CC4(C3)CN(c3ncncc3Oc3ccc(F)cc3C(=O)N(C(C)C)[C@H]3C[C@](C)(N5CCCC5)C3)C4)c21] | (D2)-2-((4-(6-((5-Ethyl-5,6,7,8- tetrahydro-1,6-naphthyridin-4-yl)oxy)- 2-azaspiro[3.3]heptan-2-yl)pyrimidin-5- yl)oxy)-5-fluoro-N-isopropyl-N- ((1r,3r)-3-methyl-3-(pyrrolidin-1- yl)cyclobutyl)benzamide |
| | (D2) | |
| 154 | 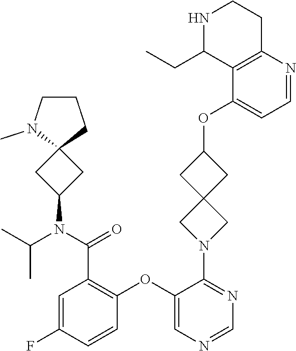 [SMILES: CCC1NCCc2nccc(OC3CC4(C3)CN(c3ncncc3Oc3ccc(F)cc3C(=O)N(C(C)C)[C@H]3C[C@@]5(CCCN5C)C3)C4)c21] | (D1)-2-((4-(6-((5-Ethyl-5,6,7,8- tetrahydro-1,6-naphthyridin-4-yl)oxy)- 2-azaspiro[3.3]heptan-2-yl)pyrimidin-5- yl)oxy)-5-fluoro-N-isopropyl-N- ((2s,4r)-5-methyl-5-azaspiro[3.4]octan- 2-yl)benzamide |
| | (D1) | |
| 155 | 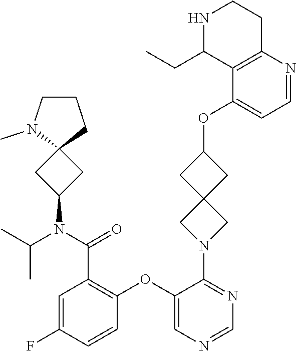 [SMILES: CCC1NCCc2nccc(OC3CC4(C3)CN(c3ncncc3Oc3ccc(F)cc3C(=O)N(C(C)C)[C@H]3C[C@@]5(CCCN5C)C3)C4)c21] | (D2)-2-((4-(6-((5-Ethyl-5,6,7,8- tetrahydro-1,6-naphthyridin-4-yl)oxy)- 2-azaspiro[3.3]heptan-2-yl)pyrimidin-5- yl)oxy)-5-fluoro-N-isopropyl-N- ((2s,4r)-5-methyl-5-azaspiro[3.4]octan- 2-yl)benzamide |
| | (D2) | |
| 156 | 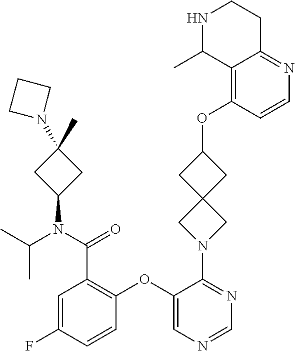 [SMILES: CC1NCCc2nccc(OC3CC4(C3)CN(c3ncncc3Oc3ccc(F)cc3C(=O)N(C(C)C)[C@H]3C[C@](C)(N5CCC5)C3)C4)c21] | (D1)-N-((1r,3r)-3-(Azetidin-1-yl)-3- methylcyclobutyl)-5-fluoro-N- isopropyl-2-((4-(6-((5-methyl-5,6,7,8- tetrahydro-1,6-naphthyridin-4-yl)oxy)- 2-azaspiro[3.3]heptan-2-yl)pyrimidin-5- yl)oxy)benzamide |
| | (D1) | |
| 157 | 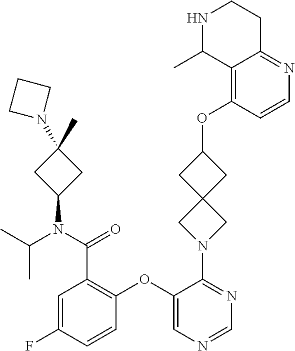 [SMILES: CC1NCCc2nccc(OC3CC4(C3)CN(c3ncncc3Oc3ccc(F)cc3C(=O)N(C(C)C)[C@H]3C[C@](C)(N5CCC5)C3)C4)c21] | (D2)-N-((1r,3r)-3-(Azetidin-1-yl)-3- methylcyclobutyl)-5-fluoro-N- isopropyl-2-((4-(6-((5-methyl-5,6,7,8- tetrahydro-1,6-naphthyridin-4-yl)oxy)- 2-azaspiro[3.3]heptan-2-yl)pyrimidin-5- yl)oxy)benzamide |
| | (D2) | |
| 158 | 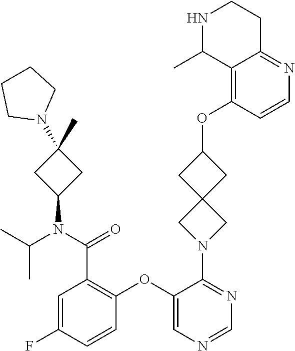 [SMILES: CC1NCCc2nccc(OC3CC4(C3)CN(c3ncncc3Oc3ccc(F)cc3C(=O)N(C(C)C)[C@H]3C[C@](C)(N5CCCC5)C3)C4)c21] | (D1)-5-Fluoro-N-isopropyl-N-((1r,3r)- 3-methyl-3-(pyrrolidin-1-yl)cyclobutyl)- 2-((4-(6-((5-methyl-5,6,7,8-tetrahydro- 1,6-naphthyridin-4-yl)oxy)-2- azaspiro[3.3]heptan-2-yl)pyrimidin-5- yl)oxy)benzamide |
| | (D1) | |
| 159 | 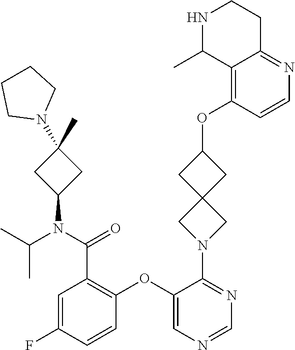 [SMILES: CC1NCCc2nccc(OC3CC4(C3)CN(c3ncncc3Oc3ccc(F)cc3C(=O)N(C(C)C)[C@H]3C[C@](C)(N5CCCC5)C3)C4)c21] | (D2)-5-Fluoro-N-isopropyl-N-((1r,3r)- 3-methyl-3-(pyrrolidin-1-yl)cyclobutyl)- 2-((4-(6-((5-methyl-5,6,7,8-tetrahydro- 1,6-naphthyridin-4-yl)oxy)-2- azaspiro[3.3]heptan-2-yl)pyrimidin-5- yl)oxy)benzamide |
| | (D2) | |
| 160 | 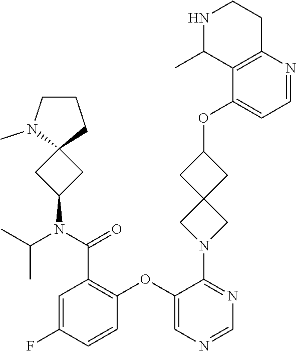 [SMILES: CC1NCCc2nccc(OC3CC4(C3)CN(c3ncncc3Oc3ccc(F)cc3C(=O)N(C(C)C)[C@H]3C[C@@]5(CCCN5C)C3)C4)c21] | (D1)-5-Fluoro-N-isopropyl-2-((4-(6-((5- methyl-5,6,7,8-tetrahydro-1,6- naphthyridin-4-yl)oxy)-2- azaspiro[3.3]heptan-2-yl)pyrimidin-5- yl)oxy)-N-((2s,4r)-5-methyl-5- azaspiro[3.4]octan-2-yl)benzamide |
| | (D1) | |
| 161 | 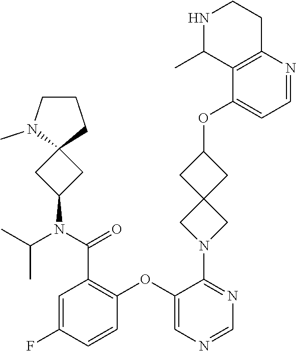 [SMILES: CC1NCCc2nccc(OC3CC4(C3)CN(c3ncncc3Oc3ccc(F)cc3C(=O)N(C(C)C)[C@H]3C[C@@]5(CCCN5C)C3)C4)c21] | (D2)-5-fluoro-N-isopropyl-2-((4-(6-((5- methyl-5,6,7,8-tetrahydro-1,6- naphthyridin-4-yl)oxy)-2- azaspiro[3.3]heptan-2-yl)pyrimidin-5- yl)oxy)-N-((2s,4r)-5-methyl-5- azaspiro[3.4]octan-2-yl)benzamide |
| | (D2) | |
| 162 | 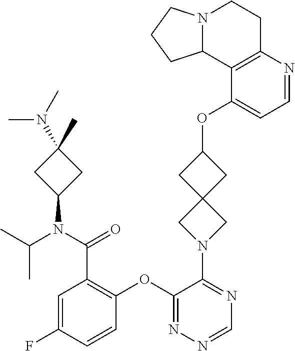 [SMILES: CC(C)N(C(=O)c1cc(F)ccc1Oc1nncnc1N1CC2(CC(Oc3ccnc4c3C3CCCN3CC4)C2)C1)[C@H]1C[C@](C)(N(C)C)C1] | (D1)-N-((1r,3r)-3-(Dimethylamino)-3- methylcyclobutyl)-5-fluoro-2-((5-(6- ((5,6,8,9,10,10a-hexahydropyrrolo[2,1- f][1,6]naphthyridin-1-yl)oxy)-2- azaspiro[3.3]heptan-2-yl)-1,2,4-triazin- 6-yl)oxy)-N-isopropylbenzamide |
| | (D1) | |
| 163 | 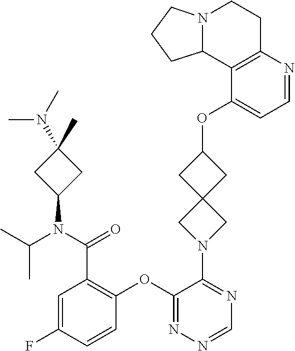 [SMILES: CC(C)N(C(=O)c1cc(F)ccc1Oc1nncnc1N1CC2(CC(Oc3ccnc4c3C3CCCN3CC4)C2)C1)[C@H]1C[C@](C)(N(C)C)C1] | (D2)-N-((1r,3r)-3-(Dimethylamino)-3- methylcyclobutyl)-5-fluoro-2-((5-(6- ((5,6,8,9,10,10a-hexahydropyrrolo[2,1- f][1,6]naphthyridin-1-yl)oxy)-2- azaspiro[3.3]heptan-2-yl)-1,2,4-triazin- 6-yl)oxy)-N-isopropylbenzamide |
| | (D2) | |
| 164 | 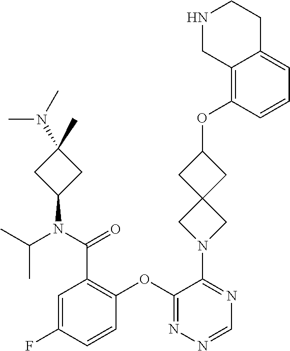 [SMILES: CC(C)N(C(=O)c1cc(F)ccc1Oc1nncnc1N1CC2(CC(Oc3cccc4c3CNCC4)C2)C1)[C@H]1C[C@](C)(N(C)C)C1] | N-((1r,3r)-3-(Dimethylamino)-3- methylcyclobutyl)-5-fluoro-N- isopropyl-2-((5-(6-((1,2,3,4- tetrahydroisoquinolin-8-yl)oxy)-2- azaspiro[3.3]heptan-2-yl)-1,2,4-triazin- 6-yl)oxy)benzamide |
| 165 | 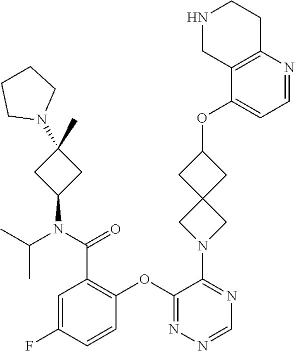 [SMILES: CC(C)N(C(=O)c1cc(F)ccc1Oc1nncnc1N1CC2(CC(Oc3ccnc4c3CNCC4)C2)C1)[C@H]1C[C@](C)(N2CCCC2)C1] | 5-Fluoro-N-isopropyl-N-((1r,3r)-3- methyl-3-(pyrrolidin-1-yl)cyclobutyl)-2- ((5-(6-((5,6,7,8-tetrahydro-1,6- naphthyridin-4-yl)oxy)-2- azaspiro[3.3]heptan-2-yl)-1,2,4-triazin- 6-yl)oxy)benzamide |
| 166 | 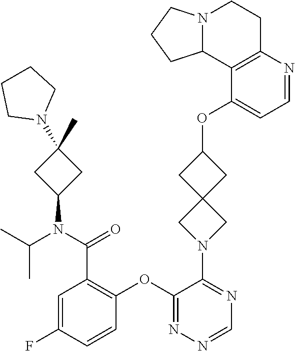 [SMILES: CC(C)N(C(=O)c1cc(F)ccc1Oc1nncnc1N1CC2(CC(Oc3ccnc4c3C3CCCN3CC4)C2)C1)[C@H]1C[C@](C)(N2CCCC2)C1] | (D1)-5-Fluoro-2-((5-(6-((5,6,8,9,10,10a- hexahydropyrrolo[2,1- f][1,6]naphthyridin-1-yl)oxy)-2- azaspiro[3.3]heptan-2-yl)-1,2,4-triazin- 6-yl)oxy)-N-isopropyl-N-((1r,3r)-3- methyl-3-(pyrrolidin-1- yl)cyclobutyl)benzamide |
| | (D1) | |
| 167 | 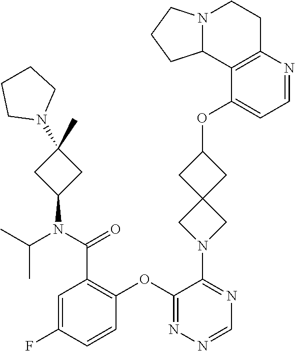 [SMILES: CC(C)N(C(=O)c1cc(F)ccc1Oc1nncnc1N1CC2(CC(Oc3ccnc4c3C3CCCN3CC4)C2)C1)[C@H]1C[C@](C)(N2CCCC2)C1] | (D2)-5-Fluoro-2-((5-(6-((5,6,8,9,10,10a- hexahydropyrrolo[2,1- f][1,6]naphthyridin-1-yl)oxy)-2- azaspiro[3.3]heptan-2-yl)-1,2,4-triazin- 6-yl)oxy)-N-isopropyl-N-((1r,3r)-3- methyl-3-(pyrrolidin-1- yl)cyclobutyl)benzamide |
| | (D2) | |
| 168 | 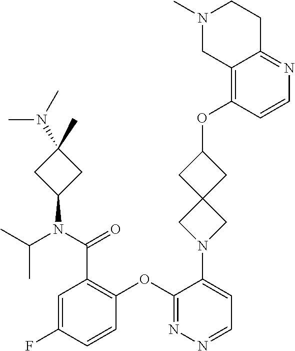 [SMILES: CC(C)N(C(=O)c1cc(F)ccc1Oc1nnccc1N1CC2(CC(Oc3ccnc4c3CN(C)CC4)C2)C1)[C@H]1C[C@](C)(N(C)C)C1] | N-((1r,3r)-3-(Dimethylamino)-3- methylcyclobutyl)-5-fluoro-N- isopropyl-2-((4-(6-((6-methyl-5,6,7,8- tetrahydro-1,6-naphthyridin-4-yl)oxy)- 2-azaspiro[3.3]heptan-2-yl)pyridazin-3- yl)oxy)benzamide |
| 169 | 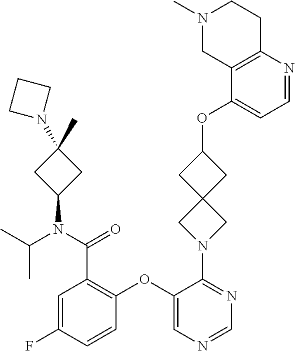 [SMILES: CC(C)N(C(=O)c1cc(F)ccc1Oc1cncnc1N1CC2(CC(Oc3ccnc4c3CN(C)CC4)C2)C1)[C@H]1C[C@](C)(N2CCC2)C1] | N-((1r,3r)-3-(Azetidin-1-yl)-3- methylcyclobutyl)-5-fluoro-N- isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 170 | 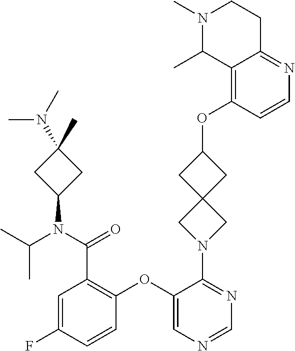 [SMILES: CC1c2c(OC3CC4(C3)CN(c3ncncc3Oc3ccc(F)cc3C(=O)N(C(C)C)[C@H]3C[C@](C)(N(C)C)C3)C4)ccnc2CCN1C] | (D2)-2-((4-(6-((5,6-Dimethyl-5,6,7,8- tetrahydro-1,6-naphthyridin-4-yl)oxy)- 2-azaspiro[3.3]heptan-2-yl)pyrimidin-5- yl)oxy)-N-((1r,3r)-3-(dimethylamino)- 3-methylcyclobutyl)-5-fluoro-N- isopropylbenzamide |
| | (D2) | |
| 171 | 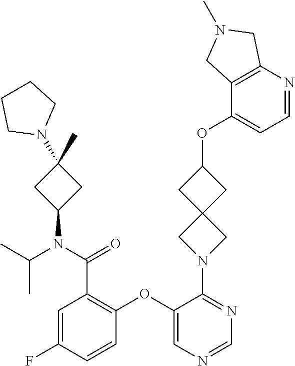 [SMILES: CC(C)N(C(=O)c1cc(F)ccc1Oc1cncnc1N1CC2(CC(Oc3ccnc4c3CN(C)C4)C2)C1)[C@H]1C[C@](C)(N2CCCC2)C1] | 5-Fluoro-N-isopropyl-N-((1r,3r)-3- methyl-3-(pyrrolidin-1-yl)cyclobutyl)-2- ((4-(6-((6-methyl-6,7-dihydro-5H- pyrrolo[3,4-b]pyridin-4-yl)oxy)-2- azaspiro[3.3]heptan-2-yl)pyrimidin-5- yl)oxy)benzamide |
| 172 | 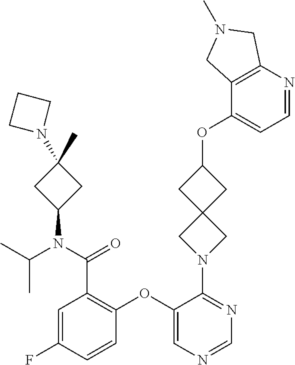 [SMILES: CC(C)N(C(=O)c1cc(F)ccc1Oc1cncnc1N1CC2(CC(Oc3ccnc4c3CN(C)C4)C2)C1)[C@H]1C[C@](C)(N2CCC2)C1] | N-((1r,3r)-3-(Azetidin-1-yl)-3- methylcyclobutyl)-5-fluoro-N- isopropyl-2-((4-(6-((6-methyl-6,7- dihydro-5H-pyrrolo[3,4-b]pyridin-4-yl) oxy)-2-azaspiro[3.3]heptan-2-yl) pyrimidin-5-yl)oxy)benzamide |
| 173 | 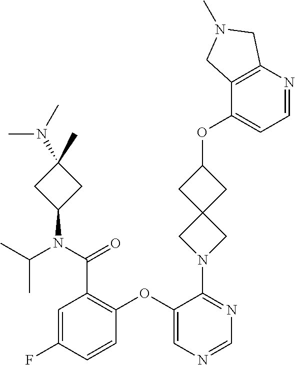 [SMILES: CC(C)N(C(=O)c1cc(F)ccc1Oc1cncnc1N1CC2(CC(Oc3ccnc4c3CN(C)C4)C2)C1)[C@H]1C[C@](C)(N(C)C)C1] | N-((1r,3r)-3-(Dimethylamino)-3- methylcyclobutyl)-5-fluoro-N- isopropyl-2-((4-(6-((6-methyl-6,7- dihydro-5H-pyrrolo[3,4-b]pyridin-4- yl)oxy)-2-azaspiro[3.3]heptan-2- yl)pyrimidin-5-yl)oxy)benzamide |
| 174 | 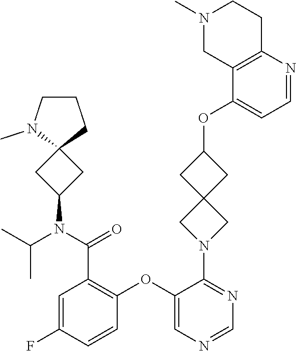 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCN2C)C1] | 5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-((2s,4r)-5-methyl-5- azaspiro[3.4]octan-2-yl)benzamide |
| 175 | 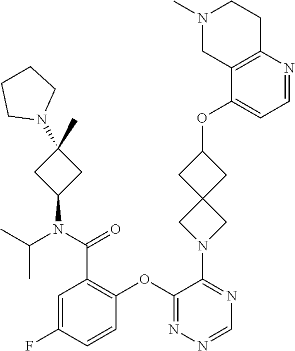 [SMILES: CC(C)N(C(=O)c1cc(F)ccc1Oc1nncnc1N1CC2(CC(Oc3ccnc4c3CN(C)CC4)C2)C1)[C@H]1C[C@](C)(N2CCCC2)C1] | 5-Fluoro-N-isopropyl-N-((1r,3r)-3- methyl-3-(pyrrolidin-1-yl)cyclobutyl)-2- ((5-(6-((6-methyl-5,6,7,8-tetrahydro- 1,6-naphthyridin-4-yl)oxy)-2- azaspiro[3.3]heptan-2-yl)-1,2,4-triazin- 6-yl)oxy)benzamide |
| 176 | 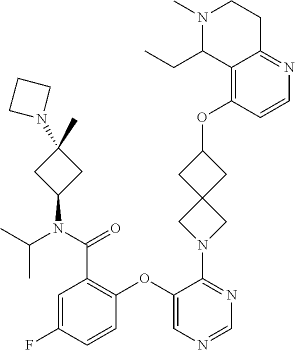 [SMILES: CCC1c2c(OC3CC4(C3)CN(c3ncncc3Oc3ccc(F)cc3C(=O)N(C(C)C)[C@H]3C[C@](C)(N5CCC5)C3)C4)ccnc2CCN1C] | (D2)-N-((1r,3r)-3-(Azetidin-1-yl)-3- methylcyclobutyl)-2-((4-(6-((5-ethyl-6- methyl-5,6,7,8-tetrahydro-1,6- naphthyridin-4-yl)oxy)-2- azaspiro[3.3]heptan-2-yl)pyrimidin-5- yl)oxy)-5-fluoro-N-isopropylbenzamide |
| | (D2) | |
| 177 | 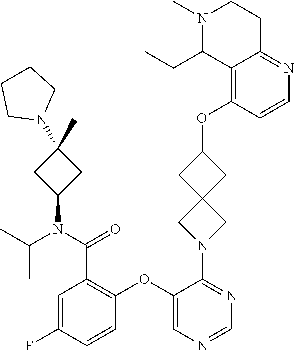 [SMILES: CCC1c2c(OC3CC4(C3)CN(c3ncncc3Oc3ccc(F)cc3C(=O)N(C(C)C)[C@H]3C[C@](C)(N5CCCC5)C3)C4)ccnc2CCN1C] | (D2)-2-((4-(6-((5-Ethyl-6-methyl- 5,6,7,8-tetrahydro-1,6-naphthyridin-4- yl)oxy)-2-azaspiro[3.3]heptan-2- yl)pyrimidin-5-yl)oxy)-5-fluoro-N- isopropyl-N-((1r,3r)-3-methyl-3- (pyrrolidin-1-yl)cyclobutyl)benzamide |
| | (D2) | |
| 178 | 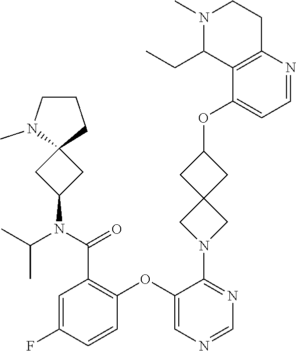 [SMILES: CCC1c2c(OC3CC4(C3)CN(c3ncncc3Oc3ccc(F)cc3C(=O)N(C(C)C)[C@H]3C[C@@]5(CCCN5C)C3)C4)ccnc2CCN1C] | (D2)-2-((4-(6-((5-Ethyl-6-methyl- 5,6,7,8-tetrahydro-1,6-naphthyridin-4- yl)oxy)-2-azaspiro[3.3]heptan-2- yl)pyrimidin-5-yl)oxy)-5-fluoro-N- isopropyl-N-((2s,4r)-5-methyl-5- azaspiro[3.4]octan-2-yl)benzamide |
| | (D2) | |
| 179 | 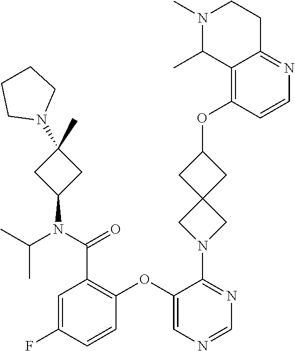 [SMILES: CC1c2c(OC3CC4(C3)CN(c3ncncc3Oc3ccc(F)cc3C(=O)N(C(C)C)[C@H]3C[C@](C)(N5CCCC5)C3)C4)ccnc2CCN1C] | (D2)-2-((4-(6-((5,6-Dimethyl-5,6,7,8- tetrahydro-1,6-naphthyridin-4-yl)oxy)- 2-azaspiro[3.3]heptan-2-yl)pyrimidin-5- yl)oxy)-5-fluoro-N-isopropyl-N- ((1r,3r)-3-methyl-3-(pyrrolidin-1- yl)cyclobutyl)benzamide |
| | (D2) | |
| 180 | 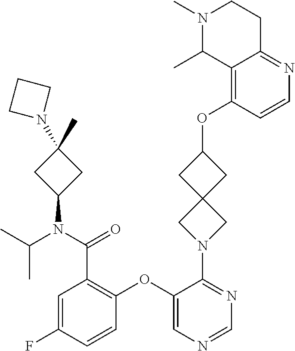 [SMILES: CC1c2c(OC3CC4(C3)CN(c3ncncc3Oc3ccc(F)cc3C(=O)N(C(C)C)[C@H]3C[C@](C)(N5CCC5)C3)C4)ccnc2CCN1C] | (D2)-N-((1r,3r)-3-(Azetidin-1-yl)-3- methylcyclobutyl)-2-((4-(6-((5,6- dimethyl-5,6,7,8-tetrahydro-1,6- naphthyridin-4-yl)oxy)-2- azaspiro[3.3]heptan-2-yl)pyrimidin-5- yl)oxy)-5-fluoro-N-isopropylbenzamide |
| | (D2) | |
| 181 | 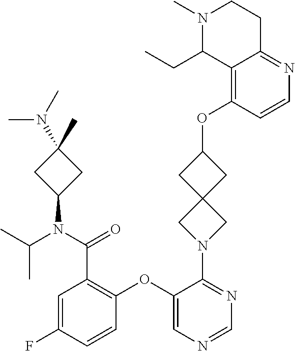 [SMILES: CCC1c2c(OC3CC4(C3)CN(c3ncncc3Oc3ccc(F)cc3C(=O)N(C(C)C)[C@H]3C[C@](C)(N(C)C)C3)C4)ccnc2CCN1C] | (D2)-N-((1r,3r)-3-(Dimethylamino)-3- methylcyclobutyl)-2-((4-(6-((5-ethyl-6- methyl-5,6,7,8-tetrahydro-1,6- naphthyridin-4-yl)oxy)-2- azaspiro[3.3]heptan-2-yl)pyrimidin-5- yl)oxy)-5-fluoro-N-isopropylbenzamide |
| | (D2) | |
| 182 | 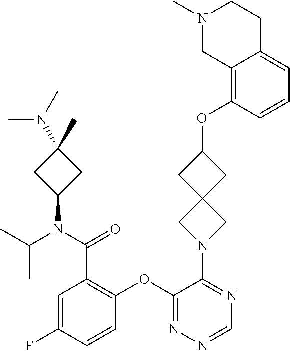 [SMILES: CC(C)N(C(=O)c1cc(F)ccc1Oc1nncnc1N1CC2(CC(Oc3cccc4c3CN(C)CC4)C2)C1)[C@H]1C[C@](C)(N(C)C)C1] | N-((1r,3r)-3-(Dimethylamino)-3- methylcyclobutyl)-5-fluoro-N- isopropyl-2-((5-(6-((2-methyl-1,2,3,4- tetrahydroisoquinolin-8-yl)oxy)-2- azaspiro[3.3]heptan-2-yl)-1,2,4-triazin- 6-yl)oxy)benzamide |
| 183 | 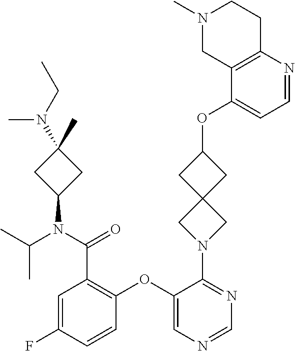 [SMILES: CCN(C)[C@]1(C)C[C@@H](N(C(=O)c2cc(F)ccc2Oc2cncnc2N2CC3(CC(Oc4ccnc5c4CN(C)CC5)C3)C2)C(C)C)C1] | N-((1r,3r)-3-(Ethyl(methyl)amino)-3- methylcyclobutyl)-5-fluoro-N- isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 184 | 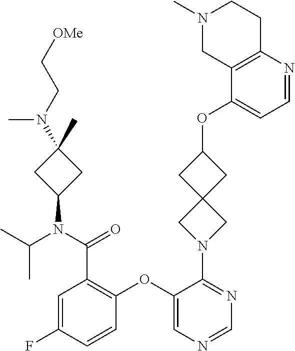 [SMILES: COCCN(C)[C@]1(C)C[C@@H](N(C(=O)c2cc(F)ccc2Oc2cncnc2N2CC3(CC(Oc4ccnc5c4CN(C)CC5)C3)C2)C(C)C)C1] | 5-Fluoro-N-isopropyl-N-((1r,3r)-3-((2- methoxyethyl)(methyl)amino)-3- methylcyclobutyl)-2-((4-(6-((6-methyl- 5,6,7,8-tetrahydro-1,6-naphthyridin-4- yl)oxy)-2-azaspiro[3.3]heptan-2- yl)pyrimidin-5-yl)oxy)benzamide |
| 185 | 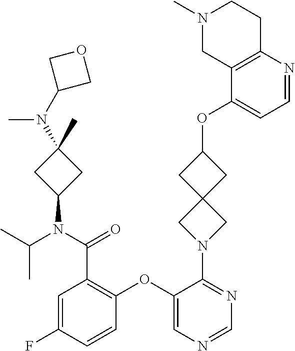 [SMILES: CC(C)N(C(=O)c1cc(F)ccc1Oc1cncnc1N1CC2(CC(Oc3ccnc4c3CN(C)CC4)C2)C1)[C@H]1C[C@](C)(N(C)C2COC2)C1] | 5-Fluoro-N-isopropyl-N-((1r,3r)-3- methyl-3-(methyl(oxetan-3- yl)amino)cyclobutyl)-2-((4-(6-((6- methyl-5,6,7,8-tetrahydro-1,6- naphthyridin-4-yl)oxy)-2- azaspiro[3.3]heptan-2-yl)pyrimidin-5- yl)oxy)benzamide |
| 186 | 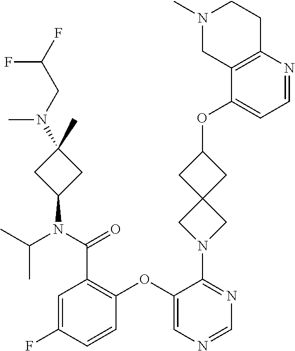 [SMILES: CC(C)N(C(=O)c1cc(F)ccc1Oc1cncnc1N1CC2(CC(Oc3ccnc4c3CN(C)CC4)C2)C1)[C@H]1C[C@](C)(N(C)CC(F)F)C1] | N-((1r,3r)-3-((2,2- Difluoroethyl)(methyl)amino)-3- methylcyclobutyl)-5-fluoro-N- isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 187 | 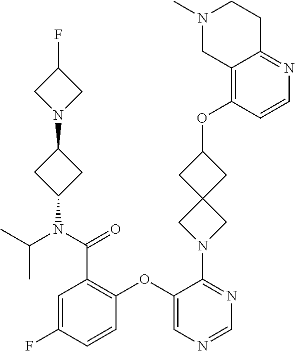 [SMILES: CC(C)N(C(=O)c1cc(F)ccc1Oc1cncnc1N1CC2(CC(Oc3ccnc4c3CN(C)CC4)C2)C1)[C@H]1C[C@H](N2CC(F)C2)C1] | S-Fluoro-N-((1r,3r)-3-(3-fluoroazetidin- 1-yl)cyclobutyl)-N-isopropyl-2-((4-(6- ((6-methyl-5,6,7,8-tetrahydro-1,6- naphthyridin-4-yl)oxy)-2- azaspiro[3.3]heptan-2-yl)pyrimidin-5- yl)oxy)benzamide |
| 188 | 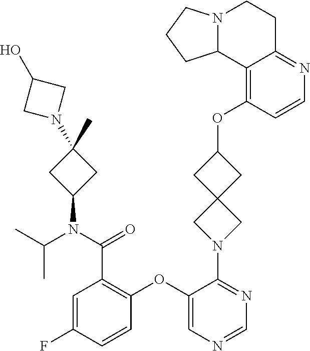 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(N2CC(O)C2)C1] | (D1)-5-Fluoro-2-((4-(6-((5,6,8,9,10,10a- hexahydropyrrolo[2,1- f][1,6]naphthyridin-1-yl)oxy)-2- azaspiro[3.3]heptan-2-yl)pyrimidin-5- yl)oxy)-N-((1r,3r)-3-(3- hydroxyazetidin-1-yl)-3- methylcyclobutyl)-N- isopropylbenzamide |
| | (D1) | |
| 189 | 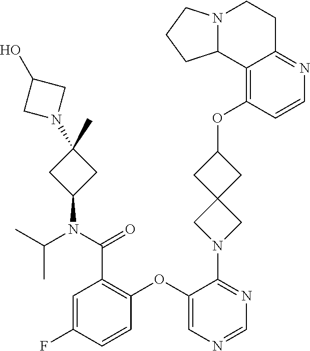 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(N2CC(O)C2)C1] | (D2)-5-Fluoro-2-((4-(6-((5,6,8,9,10,10a- hexahydropyrrolo[2,1- f][1,6]naphthyridin-1-yl)oxy)-2- azaspiro[3.3]heptan-2-yl)pyrimidin-5- yl)oxy)-N-((1r,3r)-3-(3- hydroxyazetidin-1-yl)-3- methylcyclobutyl)-N- isopropylbenzamide |
| | (D2) | |
| 190 | 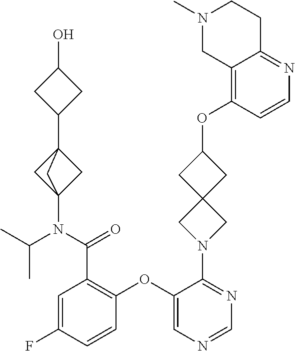 [SMILES: CC(C)N(C(=O)c1cc(F)ccc1Oc1cncnc1N1CC2(CC(Oc3ccnc4c3CN(C)CC4)C2)C1)C12CC(C3CC(O)C3)(C1)C2] | 5-Fluoro-N-(3-(3-hydroxyazetidin-1- yl)bicyclo[1.1.1]pentan-1-yl)-N- isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 191 | 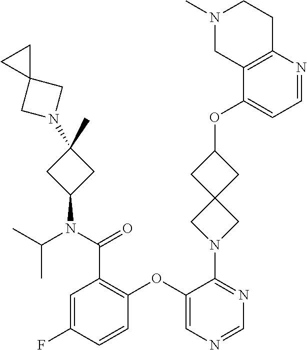 [SMILES: CC(C)N(C(=O)c1cc(F)ccc1Oc1cncnc1N1CC2(CC(Oc3ccnc4c3CN(C)CC4)C2)C1)[C@H]1C[C@](C)(N2CC3(CC3)C2)C1] | 5-Fluoro-N-isopropyl-N-((1r,3r)-3- methyl-3-(5-azaspiro[2.3]hexan-5-yl) cyclobutyl)-2-((4-(6-((6-methyl-5,6,7,8- tetrahydro-1,6-naphthyridin-4-yl)oxy)- 2-azaspiro[3.3]heptan-2-yl)pyrimidin- 5-yl)oxy)benzamide |
| 192 | 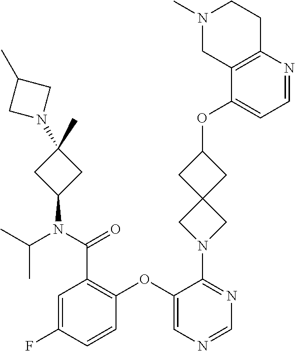 [SMILES: CC1CN([C@]2(C)C[C@@H](N(C(=O)c3cc(F)ccc3Oc3cncnc3N3CC4(CC(Oc5ccnc6c5CN(C)CC6)C4)C3)C(C)C)C2)C1] | 5-Fluoro-N-isopropyl-N-((1r,3r)-3- methyl-3-(3-methylazetidin-1- yl)cyclobutyl)-2-((4-(6-((6-methyl- 5,6,7,8-tetrahydro-1,6-naphthyridin-4- yl)oxy)-2-azaspiro[3.3]heptan-2- yl)pyrimidin-5-yl)oxy)benzamide |
| 193 | 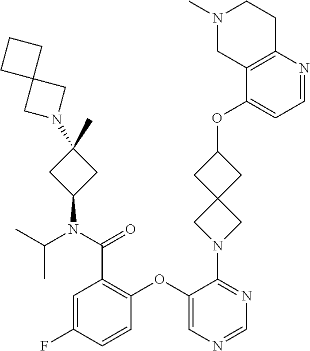 [SMILES: CC(C)N(C(=O)c1cc(F)ccc1Oc1cncnc1N1CC2(CC(Oc3ccnc4c3CN(C)CC4)C2)C1)[C@H]1C[C@](C)(N2CC3(CCC3)C2)C1] | 5-Fluoro-N-isopropyl-N-((1r,3r)-3- methyl-3-(2-azaspiro[3.3]heptan-2- yl)cyclobutyl)-2-((4-(6-((6-methyl- 5,6,7,8-tetrahydro-1,6-naphthyridin-4- yl)oxy)-2-azaspiro[3.3]heptan-2- yl)pyrimidin-5-yl)oxy)benzamide |
| 194 | 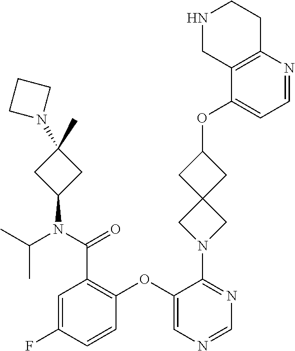 [SMILES: CC(C)N(C(=O)c1cc(F)ccc1Oc1cncnc1N1CC2(CC(Oc3ccnc4c3CNCC4)C2)C1)[C@H]1C[C@](C)(N2CCC2)C1] | N-((1r,3r)-3-(Azetidin-1-yl)-3- methylcyclobutyl)-5-fluoro-N- isopropyl-2-((4-(6-((5,6,7,8-tetrahydro- 1,6-naphthyridin-4-yl)oxy)-2- azaspiro[3.3]heptan-2-yl)pyrimidin-5- yl)oxy)benzamide |
| 195 | 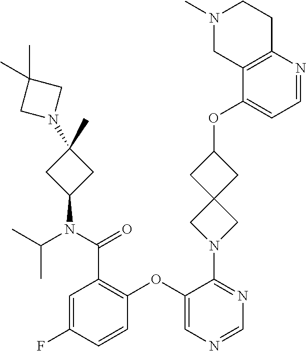 [SMILES: CC(C)N(C(=O)c1cc(F)ccc1Oc1cncnc1N1CC2(CC(Oc3ccnc4c3CN(C)CC4)C2)C1)[C@H]1C[C@](C)(N2CC(C)(C)C2)C1] | N-((1r,3r)-3-(3,3-Dimethylazetidin-1- yl)-3-methylcyclobutyl)-5-fluoro-N- isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 196 | 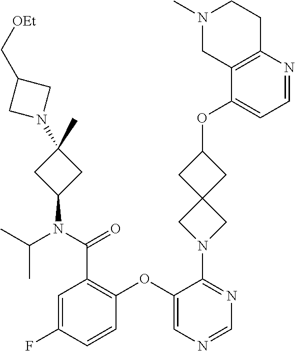 [SMILES: CCOCC1CN([C@]2(C)C[C@@H](N(C(=O)c3cc(F)ccc3Oc3cncnc3N3CC4(CC(Oc5ccnc6c5CN(C)CC6)C4)C3)C(C)C)C2)C1] | N-((1r,3r)-3-(3-(Ethoxy methyl) azetidin-1-yl)-3-methylcyclobutyl)-5- fluoro-N-isopropyl-2-((4-(6-((6-methyl- 5,6,7,8-tetrahydro-1,6-naphthyridin-4- yl)oxy)-2-azaspiro[3.3]heptan-2-yl) pyrimidin-5-yl)oxy)benzamide |
| 197 | 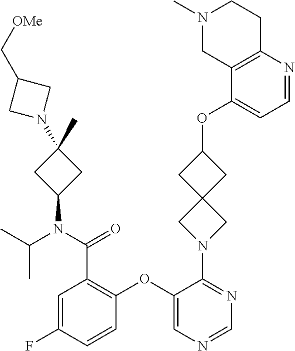 [SMILES: COCC1CN([C@]2(C)C[C@@H](N(C(=O)c3cc(F)ccc3Oc3cncnc3N3CC4(CC(Oc5ccnc6c5CN(C)CC6)C4)C3)C(C)C)C2)C1] | 5-Fluoro-N-isopropyl-N-((1r,3r)-3-(3- (methoxymethyl)azetidin-1-yl)-3- methylcyclobutyl)-2-((4-(6-((6-methyl- 5,6,7,8-tetrahydro-1,6-naphthyridin-4- yl)oxy)-2-azaspiro[3.3]heptan-2- yl)pyrimidin-5-yl)oxy)benzamide |
| 198 | 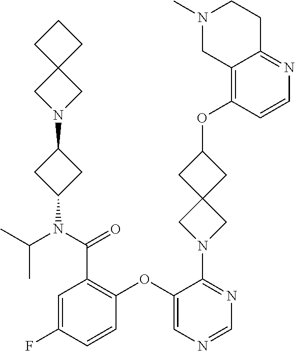 [SMILES: CC(C)N(C(=O)c1cc(F)ccc1Oc1cncnc1N1CC2(CC(Oc3ccnc4c3CN(C)CC4)C2)C1)[C@H]1C[C@H](N2CC3(CCC3)C2)C1] | N-((1r,3r)-3-(2-Azaspiro[3.3]heptan-2- yl)cyclobutyl)-5-fluoro-N-isopropyl-2- ((4-(6-((6-methyl-5,6,7,8-tetrahydro- 1,6-naphthyridin-4-yl)oxy)-2- azaspiro[3.3]heptan-2-yl)pyrimidin-5- yl)oxy)benzamide |
| 199 | 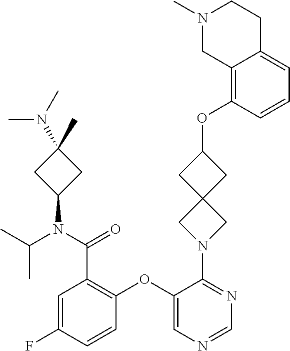 [SMILES: CC(C)N(C(=O)c1cc(F)ccc1Oc1cncnc1N1CC2(CC(Oc3cccc4c3CN(C)CC4)C2)C1)[C@H]1C[C@](C)(N(C)C)C1] | N-((1r,3r)-3-(Dimethylamino)-3- methylcyclobutyl)-5-fluoro-N- isopropyl-2-((4-(6-((2-methyl-1,2,3,4- tetrahydroisoquinolin-8-yl)oxy)-2- azaspiro[3.3]heptan-2-yl)pyrimidin-5- yl)oxy)benzamide |
| 200 | 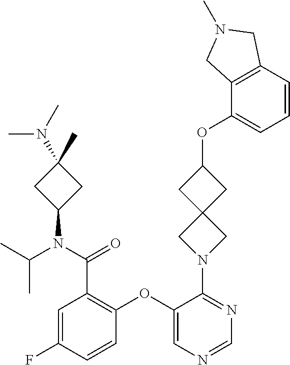 [SMILES: CC(C)N(C(=O)c1cc(F)ccc1Oc1cncnc1N1CC2(CC(Oc3cccc4c3CN(C)C4)C2)C1)[C@H]1C[C@](C)(N(C)C)C1] | N-((1r,3r)-3-(Dimethylamino)-3- methylcyclobutyl)-5-fluoro-N- isopropyl-2-((4-(6-((2-methylisoindolin- 4-yl)oxy)-2-azaspiro[3.3]heptan-2- yl)pyrimidin-5-yl)oxy)benzamide |
| 201 | 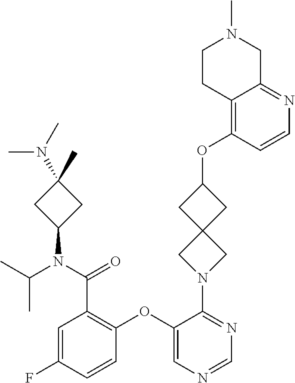 [SMILES: CC(C)N(C(=O)c1cc(F)ccc1Oc1cncnc1N1CC2(CC(Oc3ccnc4c3CCN(C)C4)C2)C1)[C@H]1C[C@](C)(N(C)C)C1] | N-((1r,3r)-3-(Dimethylamino)-3- methylcyclobutyl)-5-fluoro-N- isopropyl-2-((4-(6-((7-methyl-5,6,7,8- tetrahydro-1,7-naphthyridin-4-yl)oxy)- 2-azaspiro[3.3]heptan-2-yl)pyrimidin-5- yl)oxy)benzamide |
| 202 | 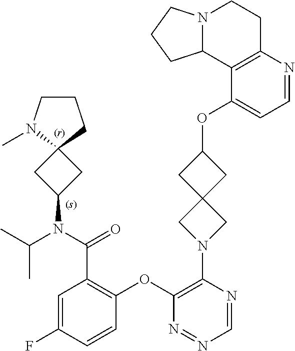 [SMILES: CC(C)N(C(=O)c1cc(F)ccc1Oc1nncnc1N1CC2(CC(Oc3ccnc4c3C3CCCN3CC4)C2)C1)[C@H]1C[C@@]2(CCCN2C)C1] | (D1)-5-Fluoro-2-((5-(6-((5,6,8,9,10,10a- hexahydropyrrolo[2,1- f][1,6]naphthyridin-1-yl)oxy)-2- azaspiro[3.3]heptan-2-yl)-1,2,4-triazin- 6-yl)oxy)-N-isopropyl-N-((2s,4r)-5- methyl-5-azaspiro[3.4]octan-2- yl)benzamide |
| | (Isomer 1) | |
| 203 | 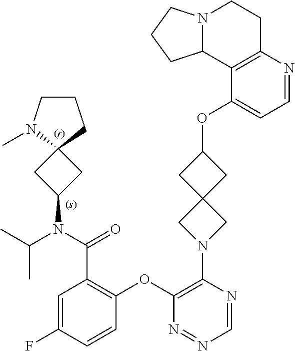 [SMILES: CC(C)N(C(=O)c1cc(F)ccc1Oc1nncnc1N1CC2(CC(Oc3ccnc4c3C3CCCN3CC4)C2)C1)[C@H]1C[C@@]2(CCCN2C)C1] | (D2)-5-Fluoro-2-((5-(6-((5,6,8,9,10,10a- hexahydropyrrolo[2,1- f][1,6]naphthyridin-1-yl)oxy)-2- azaspiro[3.3]heptan-2-yl)-1,2,4-triazin- 6-yl)oxy)-N-isopropyl-N-((2s,4r)-5- methyl-5-azaspiro[3.4]octan-2- yl)benzamide |
| | (Isomer 2) | |
| 204 | 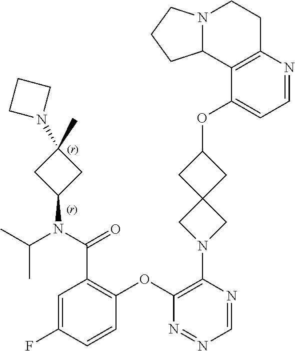 [SMILES: CC(C)N(C(=O)c1cc(F)ccc1Oc1nncnc1N1CC2(CC(Oc3ccnc4c3C3CCCN3CC4)C2)C1)[C@H]1C[C@](C)(N2CCC2)C1] | (D1)-N-((1r,3r)-3-(Azetidin-1-yl)-3- methylcyclobutyl)-5-fluoro-2-((5-(6- ((5,6,8,9,10,10a-hexahydropyrrolo[2,1- f][1,6]naphthyridin-1-yl)oxy)-2- azaspiro[3.3]heptan-2-yl)-1,2,4-triazin- 6-yl)oxy)-N-isopropylbenzamide |
| | (Isomer 1) | |
| 205 | 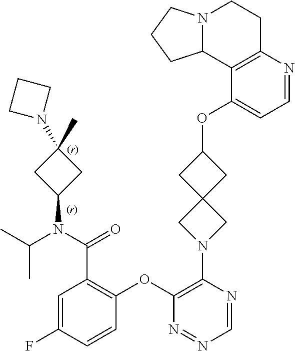 [SMILES: CC(C)N(C(=O)c1cc(F)ccc1Oc1nncnc1N1CC2(CC(Oc3ccnc4c3C3CCCN3CC4)C2)C1)[C@H]1C[C@](C)(N2CCC2)C1] | (D2)-N-((1r,3r)-3-(Azetidin-1-yl)-3- methylcyclobutyl)-5-fluoro-2-((5-(6- ((5,6,8,9,10,10a-hexahydropyrrolo[2,1- f][1,6]naphthyridin-1-yl)oxy)-2- azaspiro[3.3]heptan-2-yl)-1,2,4-triazin- 6-yl)oxy)-N-isopropylbenzamide |
| | (Isomer 2) | |
| 206 | 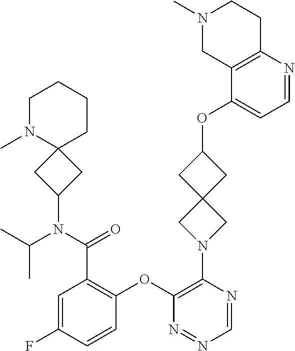 [SMILES: CC(C)N(C(=O)c1cc(F)ccc1Oc1nncnc1N1CC2(CC(Oc3ccnc4c3CN(C)CC4)C2)C1)C1CC2(CCCCN2C)C1] | (D1)-5-Fluoro-N-isopropyl-2-((5-(6-((6- methyl-5,6,7,8-tetrahydro-1,6- naphthyridin-4-yl)oxy)-2- azaspiro[3.3]heptan-2-yl)-1,2,4-triazin- 6-yl)oxy)-N-(5-methyl-5- azaspiro[3.5]nonan-2-yl)benzamide |
| | (Isomer 1) | |
| 207 | 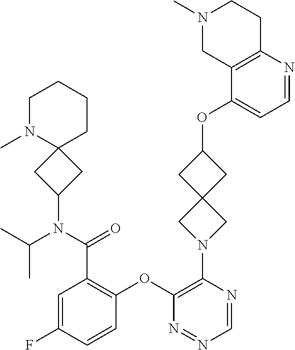 [SMILES: CC(C)N(C(=O)c1cc(F)ccc1Oc1nncnc1N1CC2(CC(Oc3ccnc4c3CN(C)CC4)C2)C1)C1CC2(CCCCN2C)C1] | (D2)-5-Fluoro-N-isopropyl-2-((5-(6-((6- methyl-5,6,7,8-tetrahydro-1,6- naphthyridin-4-yl)oxy)-2- azaspiro[3.3]heptan-2-yl)-1,2,4-triazin- 6-yl)oxy)-N-(5-methyl-5- azaspiro[3.5]nonan-2-yl)benzamide |
| | (Isomer 2) | |
| 208 | 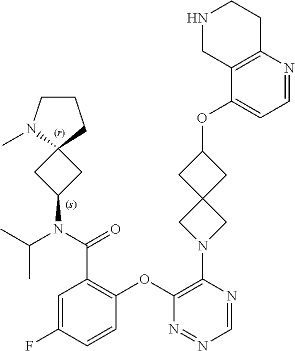 [SMILES: CC(C)N(C(=O)c1cc(F)ccc1Oc1nncnc1N1CC2(CC(Oc3ccnc4c3CNCC4)C2)C1)[C@H]1C[C@@]2(CCCN2C)C1] | 5-Fluoro-N-isopropyl-N-((2s,4r)-5- methyl-5-azaspiro[3.4]octan-2-yl)-2-((5- (6-((5,6,7,8-tetrahydro-1,6- naphthyridin-4-yl)oxy)-2- azaspiro[3.3]heptan-2-yl)-1,2,4-triazin- 6-yl)oxy)benzamide |
| 209 | 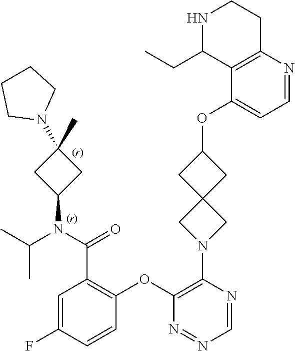 [SMILES: CCC1NCCc2nccc(OC3CC4(C3)CN(c3ncnnc3Oc3ccc(F)cc3C(=O)N(C(C)C)[C@H]3C[C@](C)(N5CCCC5)C3)C4)c21] | (D1)-2-((5-(6-((5-Ethyl-5,6,7,8- tetrahydro-1,6-naphthyridin-4-yl)oxy)- 2-azaspiro[3.3]heptan-2-yl)-1,2,4- triazin-6-yl)oxy)-5-fluoro-N-isopropyl- N-((1r,3r)-3-methyl-3-(pyrrolidin-1- yl)cyclobutyl)benzamide |
| | (Isomer 1) | |
| 210 | 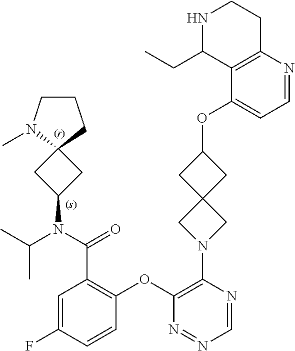 [SMILES: CCC1NCCc2nccc(OC3CC4(C3)CN(c3ncnnc3Oc3ccc(F)cc3C(=O)N(C(C)C)[C@H]3C[C@@]5(CCCN5C)C3)C4)c21] | (D1)-2-((5-(6-((5-Ethyl-5,6,7,8- tetrahydro-1,6-naphthyridin-4-yl)oxy)- 2-azaspiro[3.3]heptan-2-yl)-1,2,4- triazin-6-yl)oxy)-5-fluoro-N-isopropyl- N-((2s,4r)-5-methyl-5- azaspiro[3.4]octan-2-yl)benzamide |
| | (Isomer 1) | |
| 211 | 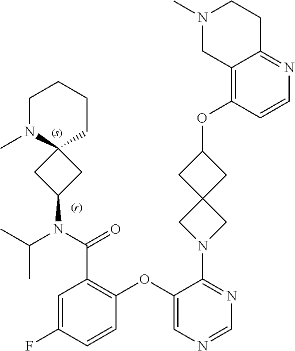 [SMILES: CC(C)N(C(=O)c1cc(F)ccc1Oc1cncnc1N1CC2(CC(Oc3ccnc4c3CN(C)CC4)C2)C1)[C@H]1C[C@]2(CCCCN2C)C1] | 5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-((2r,4s)-5-methyl-5- azaspiro[3.5]nonan-2-yl)benzamide |
| 212 | 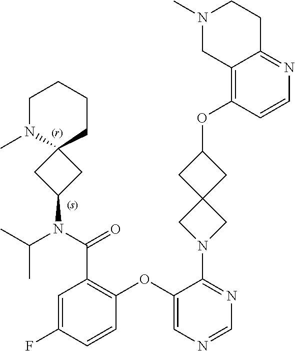 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCCN2C)C1] | 5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-((2s,4r)-5-methyl-5- azaspiro[3.5]nonan-2-yl)benzamide |
| 213 | 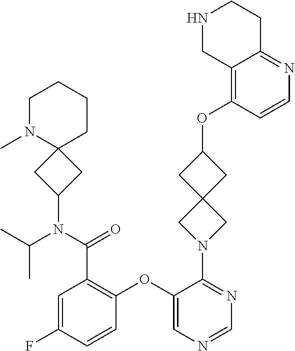 [SMILES: CC(C)N(C(=O)c1cc(F)ccc1Oc1cncnc1N1CC2(CC(Oc3ccnc4c3CNCC4)C2)C1)C1CC2(CCCCN2C)C1] | (D1)-5-Fluoro-N-isopropyl-N-(5- methyl-5-azaspiro[3.5]nonan-2-yl)-2- ((4-(6-((5,6,7,8-tetrahydro-1,6- naphthyridin-4-yl)oxy)-2- azaspiro[3.3]heptan-2-yl)pyrimidin-5- yl)oxy)benzamide |
| | (Isomer 1) | |
| 214 | 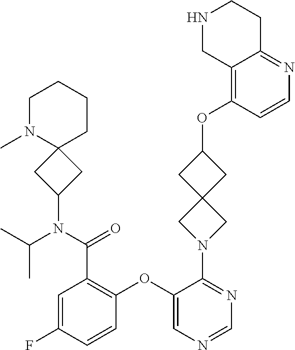 [SMILES: CC(C)N(C(=O)c1cc(F)ccc1Oc1cncnc1N1CC2(CC(Oc3ccnc4c3CNCC4)C2)C1)C1CC2(CCCCN2C)C1] | (D2)-5-Fluoro-N-isopropyl-N-(5- methyl-5-azaspiro[3.5]nonan-2-yl)-2- ((4-(6-((5,6,7,8-tetrahydro-1,6- naphthyridin-4-yl)oxy)-2- azaspiro[3.3]heptan-2-yl)pyrimidin-5- yl)oxy)benzamide |
| | (Isomer 2) | |
| 215 | 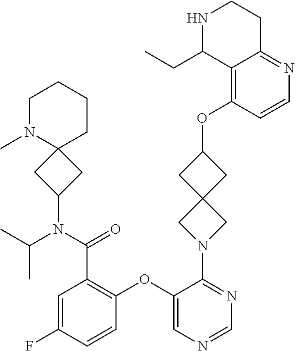 [SMILES: CCC1NCCc2nccc(OC3CC4(C3)CN(c3ncncc3Oc3ccc(F)cc3C(=O)N(C(C)C)C3CC5(CCCCN5C)C3)C4)c21] | (D1)-2-((4-(6-((5-Ethyl-5,6,7,8- tetrahydro-1,6-naphthyridin-4-yl)oxy)- 2-azaspiro[3.3]heptan-2-yl)pyrimidin-5- yl)oxy)-5-fluoro-N-isopropyl-N-(5- methyl-5-azaspiro[3.5]nonan-2- yl)benzamide |
| | (Isomer 1) | |
| 216 | 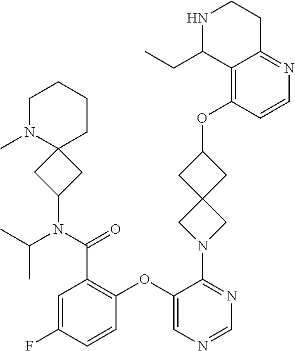 [SMILES: CCC1NCCc2nccc(OC3CC4(C3)CN(c3ncncc3Oc3ccc(F)cc3C(=O)N(C(C)C)C3CC5(CCCCN5C)C3)C4)c21] | (D2)-2-((4-(6-((5-Ethyl-5,6,7,8- tetrahydro-1,6-naphthyridin-4-yl)oxy)- 2-azaspiro[3.3]heptan-2-yl)pyrimidin-5- yl)oxy)-5-fluoro-N-isopropyl-N-(5- methyl-5-azaspiro[3.5]nonan-2- yl)benzamide |
| | (Isomer 2) | |
| 217 | 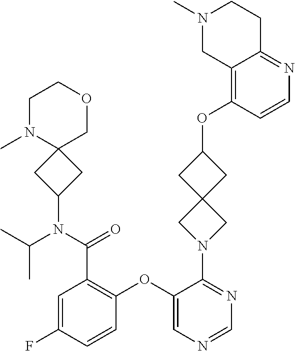 [SMILES: CC(C)N(C(=O)c1cc(F)ccc1Oc1cncnc1N1CC2(CC(Oc3ccnc4c3CN(C)CC4)C2)C1)C1CC2(COCCN2C)C1] | (D1)-5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-(5-methyl-8-oxa-5- azaspiro[3.5]nonan-2-yl)benzamide |
| | (Isomer 1) | |
| 218 | 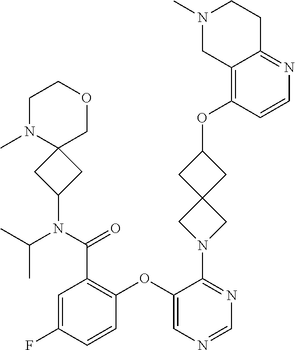 [SMILES: CC(C)N(C(=O)c1cc(F)ccc1Oc1cncnc1N1CC2(CC(Oc3ccnc4c3CN(C)CC4)C2)C1)C1CC2(COCCN2C)C1] | (D2)-5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-(5-methyl-8-oxa-5- azaspiro[3.5]nonan-2-yl)benzamide |
| | (Isomer 2) | |
| 219 | 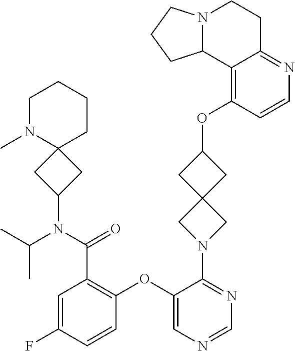 [SMILES: CC(C)N(C(=O)c1cc(F)ccc1Oc1cncnc1N1CC2(CC(Oc3ccnc4c3C3CCCN3CC4)C2)C1)C1CC2(CCCCN2C)C1] | (D1)-5-Fluoro-2-((4-(6-((5,6,8,9,10,10a- hexahydropyrrolo[2,1- f][1,6]naphthyridin-1-yl)oxy)-2- azaspiro[3.3]heptan-2-yl)pyrimidin-5- yl)oxy)-N-isopropyl-N-(5-methyl-5- azaspiro[3.5]nonan-2-yl)benzamide |
| | (Isomer 1) | |
| 220 | 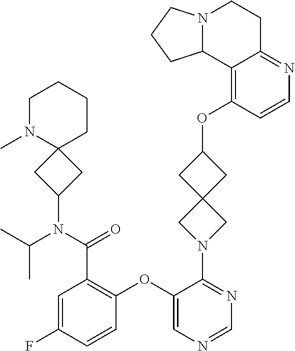 [SMILES: CC(C)N(C(=O)c1cc(F)ccc1Oc1cncnc1N1CC2(CC(Oc3ccnc4c3C3CCCN3CC4)C2)C1)C1CC2(CCCCN2C)C1] | (D2)-5-Fluoro-2-((4-(6-((5,6,8,9,10,10a- hexahydropyrrolo[2,1- f][1,6]naphthyridin-1-yl)oxy)-2- azaspiro[3.3]heptan-2-yl)pyrimidin-5- yl)oxy)-N-isopropyl-N-(5-methyl-5- azaspiro[3.5]nonan-2-yl)benzamide |
| | (Isomer 2) | |
| 221 | 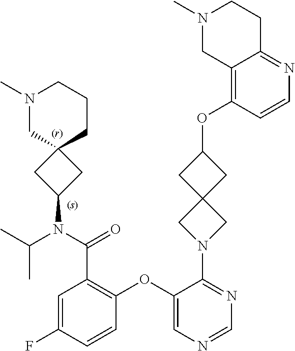 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCN(C)C2)C1] | 5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-((2s,4r)-6-methyl-6- azaspiro[3.5]nonan-2-yl)benzamide |
| 222 | 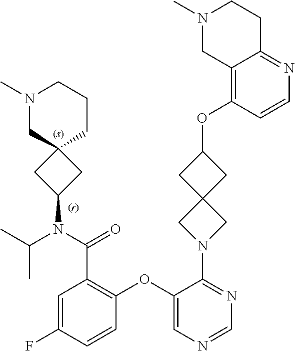 [SMILES: CC(C)N(C(=O)c1cc(F)ccc1Oc1cncnc1N1CC2(CC(Oc3ccnc4c3CN(C)CC4)C2)C1)[C@H]1C[C@]2(CCCN(C)C2)C1] | 5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-((2r,4s)-6-methyl-6- azaspiro[3.5]nonan-2-yl)benzamide |
| 223 | 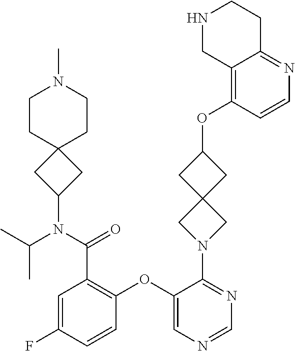 [SMILES: CC(C)N(C(=O)c1cc(F)ccc1Oc1cncnc1N1CC2(CC(Oc3ccnc4c3CNCC4)C2)C1)C1CC2(CCN(C)CC2)C1] | 5-Fluoro-N-isopropyl-N-(7-methyl-7- azaspiro[3.5]nonan-2-yl)-2-((4-(6- ((5,6,7,8-tetrahydro-1,6-naphthyridin-4- yl)oxy)-2-azaspiro[3.3]heptan-2- yl)pyrimidin-5-yl)oxy)benzamide |
| 224 | 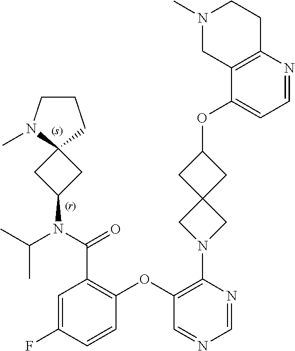 [SMILES: CC(C)N(C(=O)c1cc(F)ccc1Oc1cncnc1N1CC2(CC(Oc3ccnc4c3CN(C)CC4)C2)C1)[C@H]1C[C@]2(CCCN2C)C1] | 5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-((2r,4s)-5-methyl-5- azaspiro[3.4]octan-2-yl)benzamide |
| 225 | 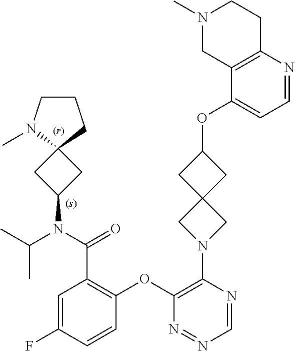 [SMILES: CC(C)N(C(=O)c1cc(F)ccc1Oc1nncnc1N1CC2(CC(Oc3ccnc4c3CN(C)CC4)C2)C1)[C@H]1C[C@@]2(CCCN2C)C1] | 5-Fluoro-N-isopropyl-2-((5-(6-((6- methyl-5,6,7,8-tetrahydro-1,6- naphthyridin-4-yl)oxy)-2- azaspiro[3.3]heptan-2-yl)-1,2,4-triazin- 6-yl)oxy)-N-((2s,4r)-5-methyl-5- azaspiro[3.4]octan-2-yl)benzamide |
| 226 | 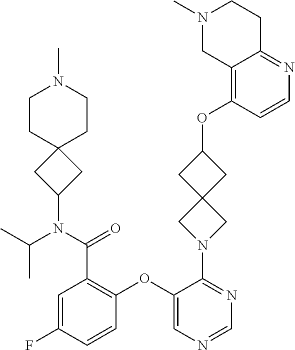 [SMILES: CC(C)N(C(=O)c1cc(F)ccc1Oc1cncnc1N1CC2(CC(Oc3ccnc4c3CN(C)CC4)C2)C1)C1CC2(CCN(C)CC2)C1] | 5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-(7-methyl-7- azaspiro[3.5]nonan-2-yl)benzamide |
| 227 | 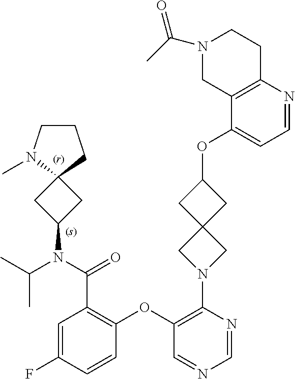 [SMILES: CC(=O)N1CCc2nccc(OC3CC4(C3)CN(c3ncncc3Oc3ccc(F)cc3C(=O)N(C(C)C)[C@H]3C[C@@]5(CCCN5C)C3)C4)c2C1] | 2-((4-(6-((6-Acetyl-5,6,7,8-tetrahydro- 1,6-naphthyridin-4-yl)oxy)-2- azaspiro[3.3]heptan-2-yl)pyrimidin-5- yl)oxy)-5-fluoro-N-isopropyl-N- ((2s,4r)-5-methyl-5-azaspiro[3.4]octan- 2-yl)benzamide |
| 228 | 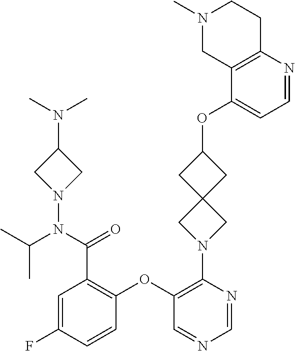 [SMILES: CC(C)N(C(=O)c1cc(F)ccc1Oc1cncnc1N1CC2(CC(Oc3ccnc4c3CN(C)CC4)C2)C1)N1CC(N(C)C)C1] | N-(3-(Dimethylamino)azetidin-1-yl)-5- fluoro-N-isopropyl-2-((4-(6-((6-methyl- 5,6,7,8-tetrahydro-1,6-naphthyridin-4- yl)oxy)-2-azaspiro[3.3]heptan-2- yl)pyrimidin-5-yl)oxy)benzamide |
| 229 | 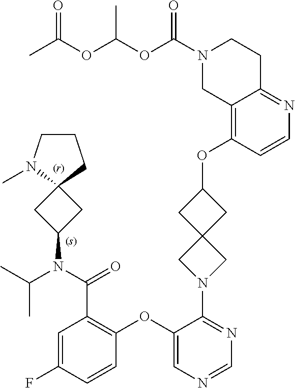 [SMILES: CC(=O)OC(C)OC(=O)N1CCc2nccc(OC3CC4(C3)CN(c3ncncc3Oc3ccc(F)cc3C(=O)N(C(C)C)[C@H]3C[C@@]5(CCCN5C)C3)C4)c2C1] | 1-Acetoxyethyl 4-((2-(5-(4-fluoro-2- (isopropyl((2s,4r)-5-methyl-5- azaspiro[3.4]octan-2- yl)carbamoyl)phenoxy)pyrimidin-4-yl)- 2-azaspiro[3.3]heptan-6-yl)oxy)-7,8- dihydro-1,6-naphthyridine-6(5H)- carboxylate |
| 230 | 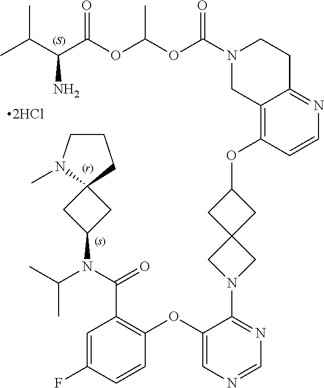 | 1-((L-Valyl)oxy)ethyl 4-((2-(5-(4- fluoro-2-(isopropyl((2s,4r)-5-methyl-5- azaspiro[3.4]octan-2- yl)carbamoyl)phenoxy)pyrimidin-4-yl)- 2-azaspiro[3.3]heptan-6-yl)oxy)-7,8- dihydro-1,6-naphthyridine-6(5H)- carboxylate dihydrochloride |
| 231 | 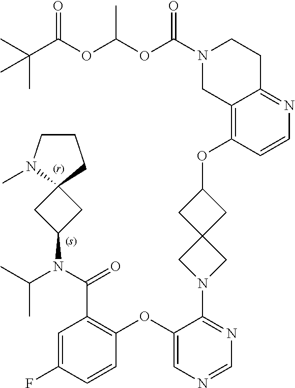 [SMILES: CC(OC(=O)N1CCc2nccc(OC3CC4(C3)CN(c3ncncc3Oc3ccc(F)cc3C(=O)N(C(C)C)[C@H]3C[C@@]5(CCCN5C)C3)C4)c2C1)OC(=O)C(C)(C)C] | 1-(Pivaloyloxy)ethyl 4-((2-(5-(4-fluoro- 2-(isopropyl((2s,4r)-5-methyl-5- azaspiro[3.4]octan-2- yl)carbamoyl)phenoxy)pyrimidin-4-yl)- 2-azaspiro[3.3]heptan-6-yl)oxy)-7,8- dihydro-1,6-naphthyridine-6(5H)- carboxylate |
| 232 | 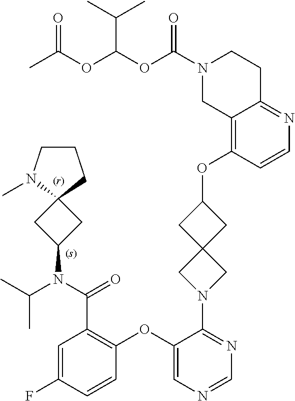 [SMILES: CC(=O)OC(OC(=O)N1CCc2nccc(OC3CC4(C3)CN(c3ncncc3Oc3ccc(F)cc3C(=O)N(C(C)C)[C@H]3C[C@@]5(CCCN5C)C3)C4)c2C1)C(C)C] | 1-Acetoxy-2-methylpropyl 4-((2-(5-(4- fluoro-2-(isopropyl((2s,4r)-5-methyl-5- azaspiro[3.4]octan-2- yl)carbamoyl)phenoxy)pyrimidin-4-yl)- 2-azaspiro[3.3]heptan-6-yl)oxy)-7,8- dihydro-1,6-naphthyridine-6(5H)- carboxylate |
| 233 | 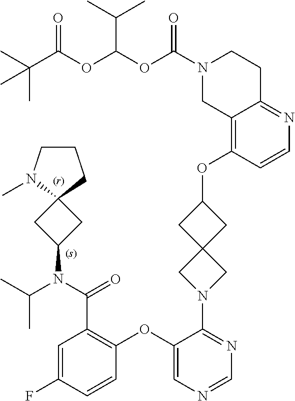 [SMILES: CC(C)C(OC(=O)N1CCc2nccc(OC3CC4(C3)CN(c3ncncc3Oc3ccc(F)cc3C(=O)N(C(C)C)[C@H]3C[C@@]5(CCCN5C)C3)C4)c2C1)OC(=O)C(C)(C)C] | 2-Methyl-1-(pivaloyloxy)propyl 4-((2- (5-(4-fluoro-2-(isopropyl((2s,4r)-5- methyl-5-azaspiro[3.4]octan-2- yl)carbamoyl)phenoxy)pyrimidin-4-yl)- 2-azaspiro[3.3]heptan-6-yl)oxy)-7,8- dihydro-1,6-naphthyridine-6(5H)- carboxylate |
| 234 | 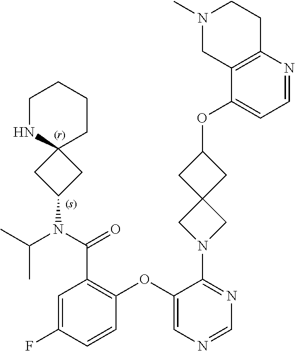 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCCN2)C1] | 5-Fluoro-N-isopropyl-2-((4-(6-((6- methyl-5,6,7,8-tetrahydro-1,6- naphthyridin-4-yl)oxy)-2- azaspiro[3.3]heptan-2-yl)pyrimidin-5- yl)oxy)-N-((2s,4r)-5- azaspiro[3.5]nonan-2-yl)benzamide |
| 235 | 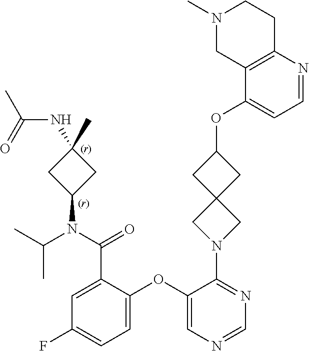 [SMILES: CC(=O)N[C@]1(C)C[C@@H](N(C(=O)c2cc(F)ccc2Oc2cncnc2N2CC3(CC(Oc4ccnc5c4CN(C)CC5)C3)C2)C(C)C)C1] | N-((1r,3r)-3-Acetamido-3- methylcyclobutyl)-5-fluoro-N- isopropyl-2-((4-(6-((6-methyl-5,6,7,8- tetrahydro-1,6-naphthyridin-4-yl)oxy)- 2-azaspiro[3.3]heptan-2-yl)pyrimidin-5- yl)oxy)benzamide |
| 236 | 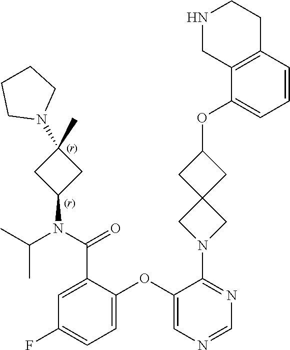 [SMILES: CC(C)N(C(=O)c1cc(F)ccc1Oc1cncnc1N1CC2(CC(Oc3cccc4c3CNCC4)C2)C1)[C@H]1C[C@](C)(N2CCCC2)C1] | 5-fluoro-N-isopropyl-N-((1r,3r)-3- methyl-3-(pyrrolidin-1-yl)cyclobutyl)-2- ((4-(6-((1,2,3,4-tetrahydroisoquinolin-8- yl)oxy)-2-azaspiro[3.3]heptan-2- yl)pyrimidin-5-yl)oxy)benzamide |
| 237 | 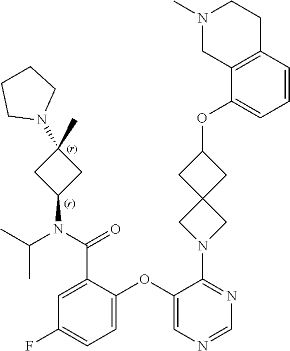 [SMILES: CC(C)N(C(=O)c1cc(F)ccc1Oc1cncnc1N1CC2(CC(Oc3cccc4c3CN(C)CC4)C2)C1)[C@H]1C[C@](C)(N2CCCC2)C1] | 5-Fluoro-N-isopropyl-2-((4-(6-((2- methyl-1,2,3,4-tetrahydroisoquinolin-8- yl)oxy)-2-azaspiro[3.3]heptan-2- yl)pyrimidin-5-yl)oxy)-N-((1r,3r)-3- methyl-3-(pyrrolidin-1- yl)cyclobutyl)benzamide |

In some embodiments, the present disclosure is directed to a compound as shown in Table 1 or a stereoisomer thereof, or a pharmaceutically acceptable salt thereof.

In some embodiments, the present disclosure is directed to a compound as shown in Table 1 or a pharmaceutically acceptable salt thereof.

In some embodiments, the present disclosure is directed to a compound as shown in Table 1.

In some embodiments, the present disclosure is directed to a compound as shown in Table 1 or a pharmaceutically acceptable salt thereof, wherein the salt is hydrochloride.

In some embodiments, a compound according to any embodiments herein (e.g., Formulae I′, I, I-a, I-b, and I-c, and Table 1) exhibits an inhibition activity against the binding of menin and MILL. In some embodiments, a compound according to any embodiments herein (e.g., Formulae I′, I, I-a, I-b, and I-c, and Table 1) exhibits an inhibition activity against the binding of menin and MILL. In some embodiments, a compound according to any embodiments herein e.g., Formulae I′, I, I-a, I-b, and I-c, and Table 1) exhibits an inhibition activity against the binding of menin and MLL which is useful in the treatment and/or prevention of one or more diseases in which menin and MLL play a role.

In some embodiments, a compound according to any embodiments herein (e.g., Formulae I′, I, I-a, I-b, and I-c, and Table 1) exhibits low hERG binding. In some embodiments, a compound according to any embodiments herein (e.g., Formulae I′, I, I-a, I-b, and I-c, and Table 1) minimizes hERG binding and is useful for the treatment of one or more diseases in which menin and MLL play a role. Without being bound to any theory, one of the primary causes of QT prolongation is thought to be blockage of the hERG potassium channel in cardiac myocytes. In some embodiments, the compounds of the present disclosure (e.g., Formulae I′, I, I-a, I-b, and I-c, and Table 1) do not significantly block the hERG potassium channel.

In some embodiments, the compounds of the present disclosure (e.g., Formulae I′, I, I-a, I-b, and I-c, and Table 1) do not significantly block the hERG potassium channel (e.g., an IC₅₀ greater than 1 μM, 5 μM, 10 μM, 15 μM, 20 μM, 25 μM, 30 μM, 35 μM, 40 μM, or 50 μM) as measured by a standard patch clamp hERG assay.

In some embodiments, the compounds of the present disclosure (e.g., Formulae I′, I, I-a, I-b, and I-c, and Table 1) do not significantly block the hERG potassium channel (e.g., a compound of the invention has an IC₅₀ greater than 1 μM, 5 μM, 10 μM, 15 μM, 20 μM, 25 μM, 30 μM, 35 μM, 40 μM, or 50 μM for the hERG potassium channel).

In some embodiments and without wishing to be bound to any theory, the present disclosure is directed to inhibitors of the menin-MLL interaction comprising a pyrrolidine moiety (e.g., Formulae I′, I, I-a, I-b, and I-c, and Table 1) where the pyrrolidine moiety has been found to reduce hERG inhibition.

In some embodiments and without wishing to be bound to any theory, the present disclosure is directed to inhibitors of the menin-MLL interaction comprising a piperidine moiety (e.g., Formulae I′, I, I-a, I-b, and I-c, and Table 1) where the piperidine moiety has been found to reduce hERG inhibition.

In some embodiments and without wishing to be bound to any theory, the present disclosure is directed to inhibitors of the menin-MLL interaction (e.g., Formulae I′, I, I-a, I-b, and I-c, and Table 1) which are resistant to metabolism. In some embodiments and without wishing to be bound to any theory, the present disclosure is directed to inhibitors of the menin-MLL interaction (e.g., Formulae I′, I, I-a, I-b, and I-c, and Table 1) which are resistant to metabolism, where the metabolites are inhibitors of the hERG potassium channel. In some embodiments and without wishing to be bound to any theory, the present disclosure is directed to inhibitors of the menin-MLL interaction (e.g., Formulae I′, I, I-a, I-b, and I-c, and Table 1) which are resistant to metabolism, where the metabolism decreases the bioavailability of the inhibitor. In some embodiments and without wishing to be bound to any theory, the present disclosure is directed to inhibitors of the menin-MLL interaction (e.g., Formulae I′, I, I-a, I-b, and I-c, and Table 1) which are resistant to metabolism, where the metabolism decreases the bioavailability of the inhibitor and the corresponding metabolites are more effective (e.g., by IC₅₀, etc.) at binding the hERG potassium channel.

In some embodiments, one or more hydrogen atoms in any of the compounds of Formula I′, I, I-a, I-b, or I-c may be replaced with one or more deuterium atoms.

Another aspect is an isotopically labeled compound of any of the formulae delineated herein. Such compounds have one or more isotopic atoms (e.g., ³H, ²H, ¹⁴C, ¹³C, ¹⁸F, ³⁵S, ³²P, ¹²⁵I, and ¹³¹I) introduced into the compound. Such compounds are useful for drug metabolism studies and diagnostics, as well as therapeutic applications. In some embodiments, the compound is an isotopic derivative of any one of the compounds described in Table 1, or a pharmaceutically acceptable salt thereof.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the present disclosure or a pharmaceutically acceptable salt or solvate thereof that contains the aforementioned deuterium atom(s) is within the scope of the disclosure. Further, substitution with deuterium (i.e., 2H) may afford certain therapeutic advantages resulting from greater metabolic stability, e.g., increased in vivo half-life or reduced dosage requirements.

For the avoidance of doubt, it is to be understood that, where in this specification a group is qualified by “described herein”, the said group encompasses the first occurring and broadest definition as well as each and all of the particular definitions for that group.

Potency can also be determined by IC₅₀ value. A compound with a lower IC₅₀ value, as determined under substantially similar conditions, is more potent relative to a compound with a higher IC₅₀ value.

Potency can also be determined by EC₅₀ value. A compound with a lower EC₅₀ value, as determined under substantially similar conditions, is more potent relative to a compound with a higher EC₅₀ value.

In some embodiments, a pair of enantiomers have similar potency.

In some embodiments, a pair of enantiomers have different potency. In specific embodiments, the enantiomer with (R) configuration is more potent than the enantiomer with (S) configuration. In other specific embodiments, the enantiomer with (S) configuration is more potent than the enantiomer with (R) configuration.

In some embodiments, a pair of isomers have similar potency.

In some embodiments, a pair of isomers have different potency. In specific embodiments, the trans isomer is more potent than the cis isomer. In specific embodiments, the cis isomer is more potent than the trans isomer.

In some embodiments, a group of diastereomers have similar potency.

In some embodiments, a group of diastereomers have different potency.

In specific embodiments, the diastereomer with (R)(R) configuration is the most potent among the group of diastereomers.

In specific embodiments, the diastereomer with (S)(S) configuration is the most potent among the group of diastereomers.

In specific embodiments, the diastereomer with (S)(R) configuration is the most potent among the group of diastereomers.

In specific embodiments, the diastereomer with (R)(S) configuration is the most potent among the group of diastereomers.

In specific embodiments, the diastereomer with (R)(R) configuration is more potent than the diastereomer with (S)(S) configuration.

In specific embodiments, the diastereomer with (S)(S) configuration is more potent than the diastereomer with (R)(R) configuration.

In specific embodiments, the diastereomer with (R)(S) configuration is more potent than the diastereomer with (S)(R) configuration.

In specific embodiments, the diastereomer with (S)(R) configuration is more potent than the diastereomer with (R)(S) configuration.

The compounds of the application are defined herein by their chemical structures and/or chemical names. Where a compound is referred to by both a chemical structure and a chemical name, and the chemical structure and chemical name conflict, the chemical structure is determinative of the compound's identity.

In another aspect, the application provides a method of synthesizing a compound disclosed herein. The synthesis of the compounds of the application can be found herein and in the Examples below. Other embodiments are a method of making a compound of any of the formulae herein using any one, or combination of, reactions delineated herein. The method can include the use of one or more intermediates or chemical reagents delineated herein.

In some embodiments, each of the intermediates prepared in the Schemes herein are considered embodiments of the present disclosure. In some embodiments, each of the intermediates prepared in the Examples herein are considered embodiments of the present disclosure. In some embodiments, each synthetic step as disclosed in the Schemes herein is separately considered as part of the present disclosure. In some embodiments, each synthetic step as disclosed in the Examples herein is separately considered as part of the present disclosure. It is appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various cycloalkyl, and heterocyclyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

For compounds of the disclosure in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the term “amino,” employed alone or in combination with other terms, refers to a group of formula —NH₂. In some embodiments, an amine can be substituted by one or more groups, e.g., —N—(C₁-C₆ alkyl)₂. In some embodiments, when two groups are attached to an amine they can be the same or different. Each group is selected independently of each other and can be each independently optionally substituted.

As used herein, the term “halogen” or “halo,” employed alone or in combination with other terms, refers to a halogen atom selected from F, Cl, I or Br. In some embodiments, “halo” refers to a halogen atom selected from F, Cl, or Br. In some embodiments, the halo substituent is F.

The term “haloalkyl” or “haloalkoxyl” refers to an alkyl or alkoxyl substituted with one or more halogen atoms. In some embodiments, the haloalkyl group is fluorinated. In some embodiments, the haloalkyl group is fluorinated only. In some embodiments, the haloalkyl group is fluoromethyl, difluoromethyl, or trifluoromethyl. In some embodiments, the haloalkyl group is trifluoromethyl. In some embodiments, the haloalkyl group is 2,2,2-trifluoroethyl. In some embodiments, the haloalkyl group is 2,2-difluoroethyl. In some embodiments, the haloalkyl group has 1 to 6 or 1 to 4 carbon atoms.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic. In some embodiments, cycloalkyl may optionally contain one or more alkenylene groups as part of the ring structure. Cycloalkyl groups can include mono- or polycyclic ring systems. Polycyclic ring systems can include fused ring systems and spirocycles. Also included in the definition of cycloalkyl are moieties that have one or more aromatic rings fused (i.e., having a bond in common with) to the cycloalkyl ring, for example, benzo or pyrido derivatives of cyclopentane, cyclopentene, cyclohexane, and the like. A heterocyclyl group that includes a fused aromatic (e.g., aryl or heteroaryl) moiety can be attached to the molecule through an atom from either the aromatic or non-aromatic portion. One or more ring-forming carbon atoms of a cycloalkyl group can be oxidized to form carbonyl linkages. In some embodiments, cycloalkyl is C₃₋₁₀ cycloalkyl, C₃₋₇ cycloalkyl, or C₅₋₆ cycloalkyl. Exemplary cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, and the like. Further exemplary cycloalkyl groups include cyclopropyl, cyclobutyl, cyclopentyl, and cyclohexyl. Additional example cycloalkyl groups, where the cycloalkyl group has a fused aryl or heteroaryl moiety, include tetrahydronaphthalen-2-yl, 2,3-dihydro-1H-inden-2-yl; 2,3,4,9-tetrahydro-1H-carbazol-7-yl; 2,6,7,8-tetrahydrobenzo[cd]indazol-4-yl; and 5,6,7,8,9,10-hexahydrocyclohepta[b]indol-3-yl. Further exemplary cycloalkyl groups include and

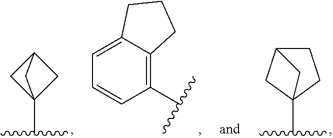

As used herein, the term “aryl,” employed alone or in combination with other terms, refers to a monocyclic or polycyclic (e.g., having 2, 3 or 4 fused rings) aromatic hydrocarbon, such as, but not limited to, phenyl, 1-naphthyl, 2-naphthyl, anthracenyl, phenanthrenyl, and the like. In some embodiments, aryl is C₆₋₁₀ aryl. In some embodiments, aryl is C₆₋₁₄ aryl. In some embodiments, the aryl group is a naphthalene ring or phenyl ring. In some embodiments, the aryl group is phenyl.

As used herein, the term “heteroaryl,” employed alone or in combination with other terms, refers to a monocyclic or polycyclic (e.g., having 2, 3 or 4 fused rings) aromatic heterocylic moiety, having one or more heteroatom ring members selected from nitrogen, sulfur and oxygen. In some embodiments, the heteroaryl group has 1, 2, 3, or 4 heteroatom ring members. In some embodiments, the heteroaryl group has 1, 2, or 3 heteroatom ring members. In some embodiments, the heteroaryl group has 1 or 2 heteroatom ring members. In some embodiments, the heteroaryl group has 1 heteroatom ring member. In some embodiments, the heteroaryl group is 5- to 10-membered or 5- to 6-membered. In some embodiments, the heteroaryl group is 5-membered. In some embodiments, the heteroaryl group is 6-membered. In some embodiments, the heteroaryl group is 9- or 10-membered bicyclic. In some embodiments, the heteroaryl is 9-member bicyclic. When the heteroaryl group contains more than one heteroatom ring member, the heteroatoms may be the same or different. The nitrogen atoms in the ring(s) of the heteroaryl group can be oxidized to form N-oxides. Example heteroaryl groups include, but are not limited to, pyridinyl, pyrimidinyl, pyrazinyl, pyridazinyl, pyrrolyl, pyrazolyl, azolyl, oxazolyl, isoxazolyl, thiazolyl, isothiazolyl, imidazolyl, furanyl, thiophenyl, triazolyl, tetrazolyl, thiadiazolyl, quinolinyl, isoquinolinyl, indolyl, benzothiophenyl, benzofuranyl, benzisoxazolyl, benzoimidazolyl, imidazo[1,2-b]thiazolyl, purinyl, triazinyl, and the like. In some embodiments, the heteroaryl group is 9H-carbazol-2-yl; 1H-benzo[d]imidazol-6-yl; 1H-indol-6-yl; 1H-indazol-6-yl; 2H-indazol-4-yl; 1H-benzo[d][1,2,3]triazol-6-yl; benzo[d]oxazol-2-yl; quinolin-6-yl; or benzo[d]thiazol-2-yl.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthyridine, indole, benzofuran, purine, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the phrase “optionally substituted” means unsubstituted or substituted. As used herein, the term “substituted” or “substituted with one or more”, means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “heterocyclyl” or “heterocycloalkyl” employed alone or in combination with other terms, refers to a non-aromatic heterocyclic ring system, which may optionally contain one or more unsaturations as part of the ring structure, and which has at least one heteroatom ring member independently selected from nitrogen, sulfur and oxygen. In some embodiments, the heterocyclyl group has 1, 2, 3, or 4 heteroatom ring members. In some embodiments, the heterocyclyl group has 1, 2, or 3 heteroatom ring members. In some embodiments, the heterocyclyl group has 1 or 2 heteroatom ring members. In some embodiments, the heterocyclyl group has 1 heteroatom ring member. When the heterocyclyl group contains more than one heteroatom in the ring, the heteroatoms may be the same or different. Example ring-forming members include CH, CH₂, C(O), N, NH, O, S, S(O), and S(═O)₂. Heterocyclyl groups can include mono- or polycyclic (e.g., having 2, 3 or 4 fused rings) ring systems. Polycyclic rings can include both fused systems and spirocycles. Also included in the definition of heterocyclyl are moieties that have one or more aromatic rings fused (i.e., having a bond in common with) to the non-aromatic ring, for example, 1, 2, 3, 4-tetrahydro-quinoline, dihydrobenzofuran and the like. A heterocyclyl group that includes a fused aromatic moiety can be attached to the molecule through an atom from either the aromatic or non-aromatic portion. The carbon atoms or heteroatoms in the ring(s) of the heterocyclyl group can be oxidized to form a carbonyl, sulfinyl, or sulfonyl group (or other oxidized linkage) or a nitrogen atom can be quaternized. In some embodiments, heterocyclyl is 5- to 10-membered, 4- to 10-membered, 4- to 7-membered, 5-membered, or 6-membered. Examples of heterocyclyl groups include 1, 2, 3, 4-tetrahydro-quinolinyl, dihydrobenzofuranyl, azetidinyl, azepanyl, pyrrolidinyl, piperidinyl, piperazinyl, morpholinyl, thiomorpholinyl, and pyranyl. Examples of heterocyclyl groups that include one or more fused aromatic groups (e.g., aryl or heteroaryl) include N-(2′-oxospiro[cyclohexane-1,3′-indolin]-6′-yl; 1,2,3,4-tetrahydroisoquinolin-6-yl; 2,3-dihydro-1H-benzo[d]imidazol-5-yl; 1,3-dihydrospiro[indene-2,3′-indolin]-6′-yl; 2,3-dihydrobenzo[d]oxazol-5-yl; 1,2-dihydroquinolin-7-yl; indolin-6-yl; spiro[cyclopentane-1,3′-indolin]-6′-yl; spiro[cyclohexane-1,3′-indolin]-6′-yl; chroman-6-yl; 3,4-dihydro-2H-benzo[b][1,4]oxazin-6-yl; and benzo[d][1,3]dioxol-5-yl.

Examples of “heterocyclyl” or “heterocycloalkyl” groups include

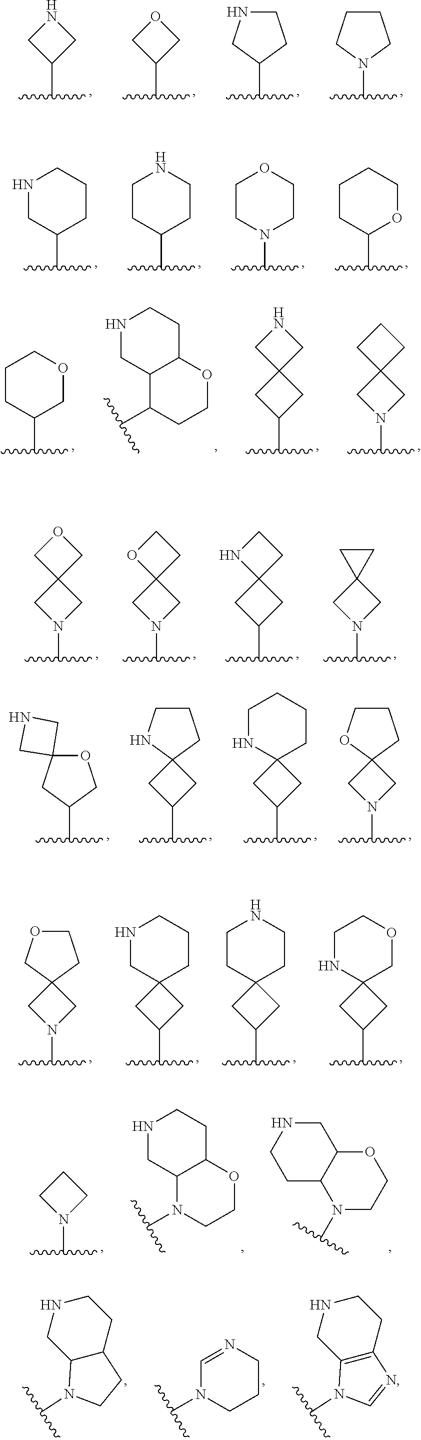

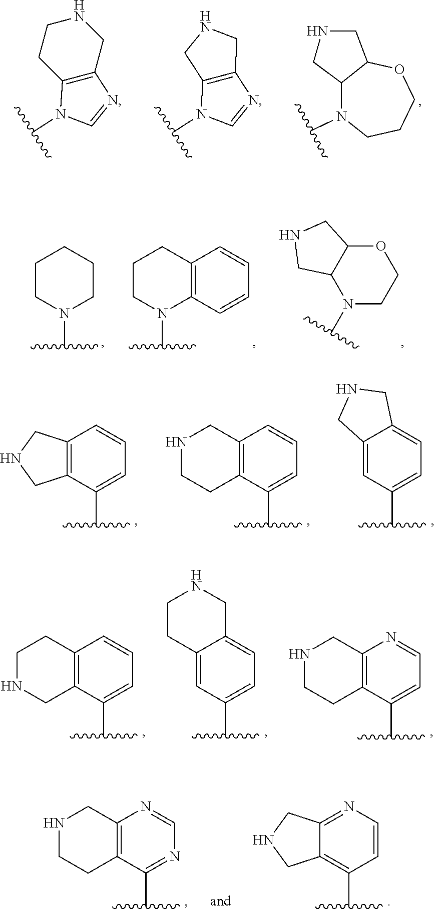

As used herein, the term “alkylene” refers to a refers to a divalent radical derived from an alkane, as exemplified, by (—CH₂—)ₙ, wherein n may be 1 to about 24. By way of example only, such groups include, but are not limited to, groups having 10 or fewer carbon atoms such as the structures —CH₂CH₂— and —CH₂CH₂CH₂CH₂—.

As used herein, the terms “Cₙ-Cₘ” and “Cₙ₋ₘ”, where each of n and m is an integer, can be used interchangeably.

As used herein, the term “cycloalkylene” refers to a cycloalkyl group wherein two hydrogens are removed to provide a divalent radical. It is understood that the two hydrogens, prior to removal, can be attached to same or different atoms in the cycloalkyl group. Examples of cycloalkylene groups include

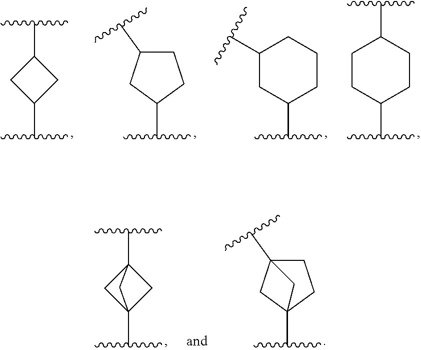

As used herein, “ [symbol] ” represents a bond to the position indicated by the corresponding variable, for example Ring A, R¹, R², R³, Z, W, Y, X³, R^{1as}, R^{1bs}, R^{1cs}, R^{3a}, R^{4a}, R^{4b}, R^{3a′}, R^{3b′}, R^{3c′}, R^{4a′}, or R^{4b′}.

As used in the embodiments describing Ring A, when Ring A is

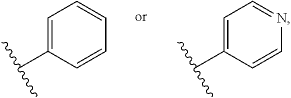 [SMILES: *c1ccccc1 ; "or" ; *c1ccncc1]

it is intended that the position of the substituents can be at any position allowed by the available valences according to general formulae I′, I, I-a, I-b, and I-c, or a stereoisomer, or a pharmaceutically acceptable salt thereof, e.g.

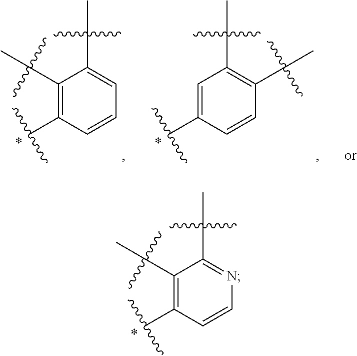

wherein * indicates attachment to Z.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R and S sequencing rules of Cahn and Prelog, or by the manner in which the molecule rotates the plane of polarised light and designated as dextrorotatory or levorotatory (i.e., as (+) or (−) isomers respectively). A chiral compound can exist as either individual enantiomer or as a mixture thereof. A mixture containing equal proportions of the enantiomers is called a “racemic mixture”. The compounds described herein can be asymmetric (e.g., having one or more stereocenters). All stereoisomers, such as enantiomers and diastereoisomers, are intended unless otherwise indicated. Where a compound name or structure is silent with respect to the stereochemistry of a stereocenter, all possible configurations at the stereocenter are intended. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

When the compounds of the disclosure contain a chiral center, the compounds can be any of the possible stereoisomers. In compounds with a single chiral center, the stereochemistry of the chiral center can be (R) or (S). In compounds with two chiral centers, the stereochemistry of the chiral centers can each be independently (R) or (S) so the configuration of the chiral centers can be (R) and (R), (R) and (S); (S) and (R), or (S) and (S). In compounds with three chiral centers, the stereochemistry each of the three chiral centers can each be independently (R) or (S) so the configuration of the chiral centers can be (R), (R) and (R); (R), (R) and (S); (R), (S) and (R); (R), (S) and (S); (S), (R) and (R); (S), (R) and (S); (S), (S) and (R); or (S), (S) and (S).

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diasteriomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

As used herein, the terms “E1” and “E2” correspond to first and second enantiomers to elute from a chiral chromatography column as discussed in the Examples.

As used herein, the terms “D1” and “D2” correspond to first and second diastereomers to elute from a chiral chromatography column as discussed in the Examples.

When a disclosed compound is named or depicted without indicating the stereochemistry of one or more stereocenters, each of the stereoisomers resulting from the possible stereochemistries at the undefined stereocenter(s) are intended to be encompassed. For example, if a stereocenter is not designated as R or S, then either or both are intended.

Compounds of the disclosure also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, amide-imidic acid pairs, enamine—imine pairs, and annular forms where a proton can occupy two or more positions of a heterocyclic system, for example, 1H- and 3H-imidazole, 1H-, 2H- and 4H-1, 2,4-triazole, 1H- and 2H-isoindole, and 1H- and 2H-pyrazole. Tautomeric forms can be in equilibrium or sterically locked into one form by appropriate substitution.

Compounds of the disclosure can also include all isotopes of atoms occurring in the intermediates or final compounds. Isotopes include those atoms having the same atomic number but different mass numbers. Isotopes of constituent atoms of the compounds of the disclosure can be present in natural or non-natural abundance. Examples of isotopes of hydrogen include deuterium and tritium. In some embodiments, the compounds of the disclosure are deuterated, meaning at least one deuterium atom is present in the place of a hydrogen atom. In some embodiments, 1, 2, 3, 4, 5, 6, 7, or 8 hydrogens in a compound of the disclosure are replaced by deuterium. Methods for replacing hydrogen with deuterium in a molecule are known in the art.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified (e.g., in the case of purine rings, unless otherwise indicated, when the compound name or structure has the 9H tautomer, it is understood that the 7H tautomer is also encompassed).

All compounds, and pharmaceutically acceptable salts thereof, can be found together with other substances such as water and solvents (e.g., hydrates and solvates) or can be isolated.

It will be understood that the compounds of the present disclosure and any pharmaceutically acceptable salts thereof, comprise stereoisomers, mixtures of stereoisomers, polymorphs of all isomeric forms of said compounds.

In some embodiments, the compounds of the disclosure, or salts thereof, or crystalline forms of any of the aforementioned, are purified or substantially isolated. By “substantially isolated” is meant that the compound is at least partially or substantially separated from the environment in which it was formed or detected. Partial separation can include, for example, a composition enriched in a compound of the disclosure. Substantial separation can include compositions containing at least about 50%, at least about 60%, at least about 70%, at least about 80%, at least about 90%, at least about 95%, at least about 97%, or at least about 99% by weight of the compounds of the disclosure, or salt thereof. In some embodiments, the compounds of the disclosure, or salts thereof, or crystalline forms of any of the aforementioned, can be prepared with a purity of about 75% or more, 80% or more, 85% or more, 90% or more, 95% or more, 98% or more, or 99% or more.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature,” as used herein, are understood in the art, and refer generally to a temperature, e.g., a reaction temperature, that is about the temperature of the room in which the reaction is carried out, for example, a temperature from about 20° C. to about 30° C.

The present disclosure also includes pharmaceutically acceptable salts of the compounds described herein. As used herein, “pharmaceutically acceptable salts” or “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two, generally, non-aqueous media like ether, ethyl acetate, alcohols (e.g., methanol, ethanol, iso-propanol, or butanol) or acetonitrile (MeCN) are preferred.

The compounds disclosed herein include the compounds themselves, as well as their salts, their solvates, and their prodrugs, if applicable. A salt, for example, can be formed between an anion and a positively charged group (e.g., protonated amino) on a compound of this disclosure. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoracetate). The term “pharmaceutically acceptable anion” refers to an anion suitable for forming a pharmaceutically acceptable salt. Likewise, a salt can also be formed between a cation and a negatively charged group (e.g., carboxylate) on a compound of this disclosure. Suitable cations include sodium ion, potassium ion, magnesium ion, calcium ion, and an ammonium cation such as tetramethylammonium ion. The compounds of this disclosure also include those salts containing quaternary nitrogen atoms. Examples of prodrugs include esters and other pharmaceutically acceptable derivatives, which, upon administration to a subject, are capable of providing active compounds of this disclosure.

Additionally, physiologically acceptable, i.e., pharmaceutically compatible, salts can be salts of the compounds disclosed herein with inorganic or organic acids. Preference is given to salts with inorganic acids, such as, for example, hydrochloric acid, hydrobromic acid, phosphoric acid or sulphuric acid, or to salts with organic carboxylic or sulphonic acids, such as, for example, acetic acid, trifluoroacetic acid, propionic acid, maleic acid, fumaric acid, malic acid, citric acid, tartaric acid, lactic acid, benzoic acid, or methanesulphonic acid, ethanesulphonic acid, benzenesulphonic acid, toluenesulphonic acid or naphthalenesulphonic acid.

Other pharmaceutically compatible salts which may be mentioned are salts with customary bases, such as, for example, alkali metal salts (for example sodium or potassium salts), alkaline earth metal salts (for example calcium or magnesium salts) or ammonium salts, derived from ammonia or organic amines, such as, for example, diethylamine, triethylamine, ethyldiisopropylamine, procaine, dibenzylamine, N-methylmorpholine, dehydroabietylamine or methylpiperidine.

As used herein, “pharmaceutically acceptable salts” can refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts can include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, or an alkaline earth metal ion, e.g., an aluminum ion; or coordinates with an organic base such as ethanolamine, diethanolamine, triethanolamine, tromethamine, N-methylglucamine, diethylamine, diethylaminoethanol, ethylenediamine, imidazole, lysine, arginine, morpholine, 2-hydroxyethylmorpholine, dibenzylethylenediamine, trimethylamine, piperidine, pyrrolidine, benzylamine, tetramethylammonium hydroxide and the like.

It should be understood that all references to pharmaceutically acceptable salts include solvent addition forms (solvates) or crystal forms (polymorphs) as defined herein, of the same salt.

It is to be understood that, unless otherwise stated, any description of a method of treatment or prevention includes use of the compounds to provide such treatment or prevention as is described herein. It is to be further understood, unless otherwise stated, any description of a method of treatment or prevention includes use of the compounds to prepare a medicament to treat or prevent such condition. The treatment or prevention includes treatment or prevention of human or non-human animals including rodents and other disease models.

It is to be understood that, unless otherwise stated, any description of a method of treatment includes use of the compounds to provide such treatment as is described herein. It is to be further understood, unless otherwise stated, any description of a method of treatment includes use of the compounds to prepare a medicament to treat such condition. The treatment includes treatment of human or non-human animals including rodents and other disease models.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

The compounds of the present disclosure may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at a sulfonylurea group.

Accordingly, the present disclosure includes those compounds of the present disclosure as defined hereinbefore when made available by organic synthesis and when made available within the human or animal body by way of cleavage of a prodrug thereof. Accordingly, the present disclosure includes those compounds of the present disclosure that are produced by organic synthetic means and also such compounds that are produced in the human or animal body by way of metabolism of a precursor compound, that is a compound of the present disclosure may be a synthetically-produced compound or a metabolically-produced compound.

A suitable pharmaceutically acceptable prodrug of a compound of the present disclosure is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of the present disclosure that possesses a hydroxy group is, for example, an in vivo cleavable ester or ether thereof. An in vivo cleavable ester or ether of a compound of the present disclosure containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N-(C₁-C₆ alkyl)₂ carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4—(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of the present disclosure that possesses a carboxy group is, for example, an in vivo cleavable amide thereof, for example an amide formed with an amine such as ammonia, a C₁-C₄ alkylamine such as methylamine, a (C₁-C₄ alkyl)₂ amine such as dimethylamine, N-ethyl N-methylamine or diethylamine, a C₁-C₄ alkoxy C₂-C₄ alkylamine such as 2 methoxyethylamine, a phenyl C₁-C₄ alkylamine such as benzylamine and amino acids such as glycine or an ester thereof.

A suitable pharmaceutically acceptable prodrug of a compound of the present disclosure that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl, and 4—(C₁-C₄ alkyl)piperazin-1-ylmethyl.

The in vivo effects of a compound of the present disclosure may be exerted in part by one or more metabolites that are formed within the human or animal body after administration of a compound of the present disclosure. As stated hereinbefore, the in vivo effects of a compound of the present disclosure may also be exerted by way of metabolism of a precursor compound (a prodrug).

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “compound of the disclosure” refers to those compounds which are disclosed herein, both generically and specifically.

### Synthesis

In some aspects, the present disclosure provides a method of preparing a compound disclosed herein.

In some aspects, the present disclosure provides a method of preparing a compound, comprising one or more steps as described herein.

In some aspects, the present disclosure provides a compound obtainable by, or obtained by, or directly obtained by a method for preparing a compound described herein.

In some aspects, the present disclosure provides an intermediate being suitable for use in a method for preparing a compound described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl (Bn). The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively, an acyl group such as a tert butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively, an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of the present disclosure has been synthesized by any one of the processes defined herein, the processes may then further comprise the additional steps of: (i) removing any protecting groups present; (ii) converting the compound of the present disclosure into another compound of the present disclosure; (iii) forming a pharmaceutically acceptable salt, hydrate or solvate thereof; and/or (iv) forming a prodrug thereof.

The resultant compounds of the present disclosure can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilizing the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognize which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesized by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons). The compounds of the disclosure can be synthesized by the methods described in Schemes 1-11 below. The synthesis of various hydroxyl-substituted heterocycles is well documented in the literature and can be synthesized by known literature methods. The depicted intermediates may also be available as commercial reagents from numerous vendors.

The compounds of the present disclosure can be prepared according to the following general procedures illustrated in the following Schemes. It will be appreciated that, although the general methods may depict the synthesis of certain compounds of the present disclosure, the following general methods, and other methods known to one of ordinary skill in the art, can be applied to all compounds and subclasses and species of each of these compounds, as described herein. The variables, where used in the following General Schemes, can be replaced to match the variable or moieties of any of the Formulae and Compounds disclosed herein as appropriate.

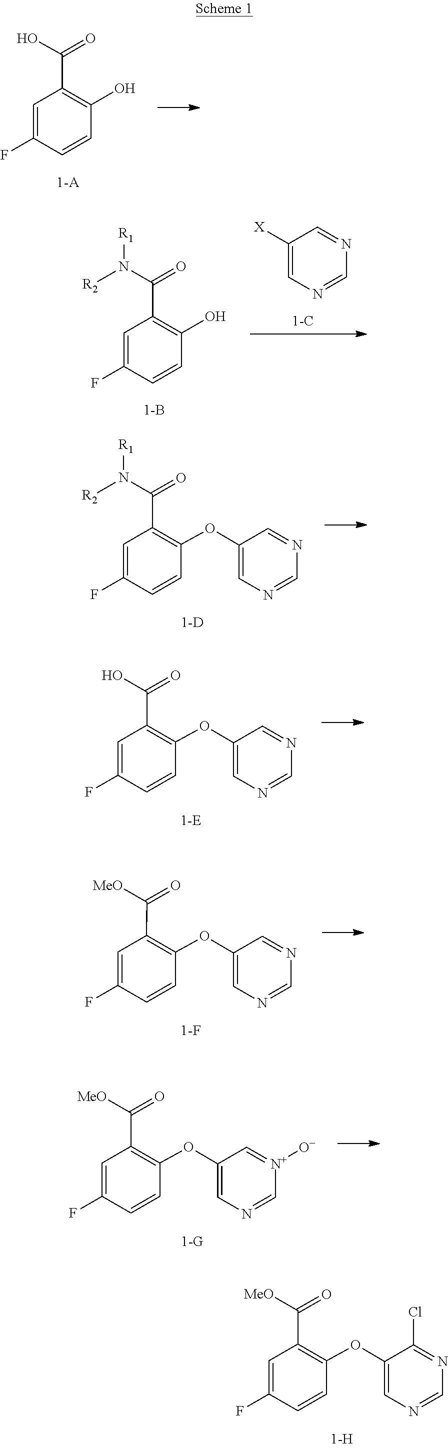

Scheme 1 illustrates the general methods for preparing the key intermediate 1-H. The carboxylic acid 1-A can be converted initially to the amide 1-B. Many different conditions have been developed to achieve this type of transformation, and are well-known to those in the art. For instance, carboxylic acid 1-A can be converted to an intermediate acid chloride (not shown), using thionyl chloride, in a neutral solvent like CH₂Cl₂ or THF, and that acid chloride can then be reacted with a suitable amine to afford amide 1-B. Typically, N,N-dimethylamine is the amine of choice for this particular chemistry, which affords the N,N-dimethyl amide (1-B; R₁ and R₂═CH₃). Amide 1-B is then reacted with a 5-halopyrimidine (1-C), such as 5-bromopyrimidine or 5-iodopyrimidine, to afford the corresponding diaryl ether 1-D. As an example, amide 1-B can be reacted with 5-bromopyrimidine in the presence of a base, for instance cesium carbonate, in a neutral solvent such as dimethylacetamide, to afford 1-D. The amide group of compound 1-D is subsequently saponified to the corresponding carboxylic acid 1-E. Typically, this transformation is accomplished using LiOH, NaOH or KOH, in an aqueous solvent system, such as methanol/water or THF/water. Heat can be used to increase the reaction rate. The carboxylic acid is then converted to ester 1-F. The transformation of a carboxylic acid to an ester is usually achieved by reacting the carboxylic acid with an alcohol, such as methanol or ethanol, in the presence of an acid catalyst like HCl or H₂SO₄. Typically, the alcohol is used as the solvent, and heat can be used to increase the reaction rate. Alternatively, 1-E can be O-alkylated using methyl iodide in the presence of an acid scavenger, oftentimes K₂CO₃, to afford 1-F. Reaction of 1-F with a suitable oxidizing agent, such as urea hydroperoxide in the presence of trifluoroacetic anhydride, or meta-chloroperoxybenzoic acid (mCPBA), in a suitable solvent, typically CH₂Cl₂ or THF, affords the pyrimidine N-oxide 1-G. This then can be chlorinated to afford compound 1-H using an appropriate chlorinating agent, typically POCl₃ or oxalyl chloride, in the presence of an acid scavenger, usually Et₃N, (i-Pr)₂NEt, in a neutral solvent like CH₂Cl₂, ethyl acetate (EtOAc), or isopropyl acetate.

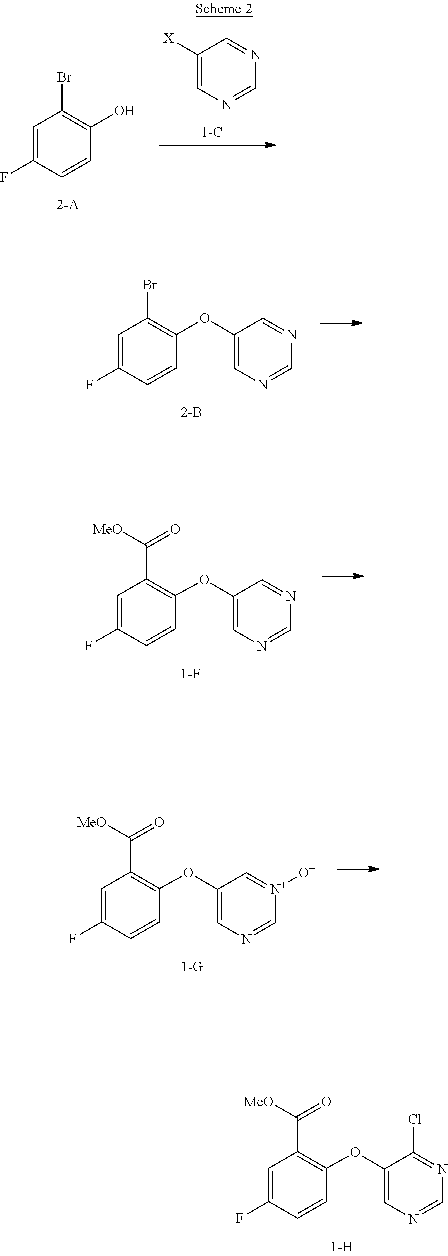

Scheme 2 illustrates an alternative method to prepare the key intermediate 1-H. A suitably substituted 2-halophenol, for instance 4-fluoro-2-bromophenol (2-A), is reacted with a 5-halopyrimidine (1-C), for example 5-bromopyrimidine or 5-iodopyrimidine, to afford the corresponding diaryl ether (2-B). This reaction is typically conducted in the presence of an acid scavenger like Na₂CO₃, K₂CO₃, Cs₂CO₃, K₃PO₄, Et₃N (triethylamine) or (i-Pr)₂NEt (diisopropylethylamine, also known as Hunig's base), in a suitable, neutral solvent, such as DMA (dimethyl acetamide) or NMP (N-methylpyrrolidinone). In some instances, this transformation can be facilitated by addition of an appropriate catalyst, typically Cu₂O or CuI, in the presence of a suitable ligand, for example rel-(1R,2R)—N¹,N²-bis(2-pyridinylmethylene)-1,2-cyclohexanediamine or 3,4,7,8-tetramethyl-1,10-phenanthroline. Conversion of compound 2-B to compound 1-F can be readily achieved by carbonylation under Pd catalysis, in the presence of an acid scavenger and suitable alcohol (typically used as the solvent in the reaction). Oftentimes, Pd(dppf)Cl₂ (dppf=1,1-bis(diphenylphosphino)ferrocene) is used as the palladium catalyst, but other catalyst systems, such as Pd(Ph₃P)₂Cl₂ (Ph₃P=triphenylphosphine) or Pd(OAc)₂ with Ph₃P, can also be used. Commonly, Et₃N or (i-Pr)₂NEt are used as the acid scavengers, and methanol is used as the solvent. These reactions are most typically run under an atmosphere of CO, but other CO sources, for example oxalic acid, can be used in some instances. Reaction of 1-F under oxidizing conditions, such as those described in Scheme 1, delivers the N-oxide derivative 1-G, which can be chlorinated, according to the general conditions described in Scheme 1, to afford the key intermediate 1-H. It will be appreciated by those of skill in the art that compound 1-H is a flexible intermediate that can be converted into the compounds of the present invention in a variety of ways.

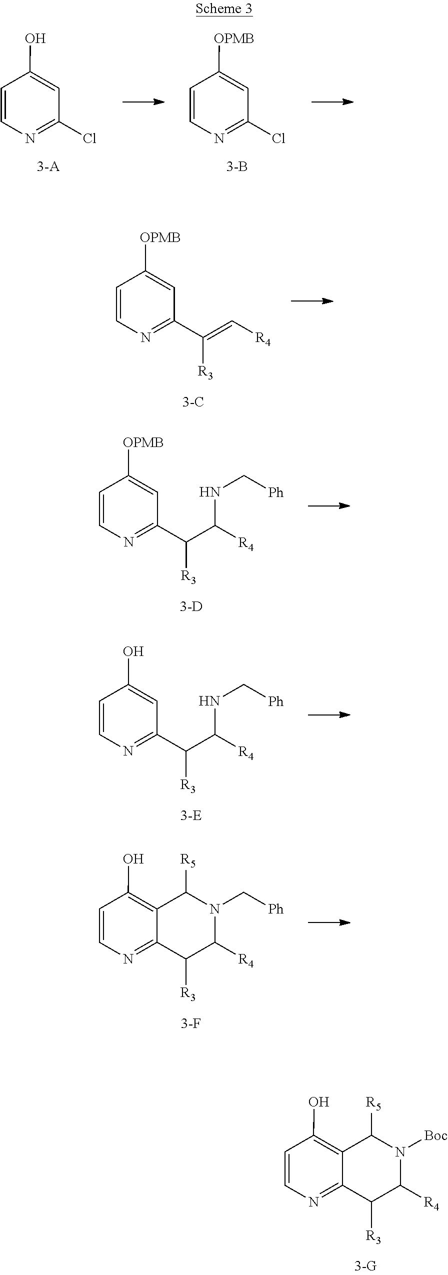

Scheme 3 illustrates the general method for the preparation of key intermediates like compound 3-G. A suitable 2-halo-4-hydroxypyridine, for instance 2-chloro-4-hydroxypyridine (3-A), is reacted with an appropriate alkylating or acylating agent, for instance 4-methoxybenzyl chloride, to afford the protected derivative 3-B. The protecting group is selected so that it can be removed selectively when desired. Note that, in some instances, the protecting group might attach to nitrogen, to afford an N-substituted 4-pyridone derivative (not shown). This would be considered acceptable, as long as the position of the protecting group (O vs. N) does not interfere with the ensuing chemistry. Compound 3-B is subsequently transformed to compound 3-C. Typically, this transformation is accomplished by reacting 3-B with a suitable olefinic boron reagent, for instance potassium vinyltrifluoroborate, vinylboronic acid, vinylboronic acid pinacol ester (also known as 2-vinyl-4,4,5,5-tetramethyl-1,3,2-dioxaborolane), isopropenylboronic acid, isopropenylboronic acid pinacol ester (also known as 2-isopropenyl-4,4,5,5-tetramethyl-1,3,2-dioxaborolane), propenylboronic acid, or propenylboronic acid pinacol ester (also known as 4,4,5,5-tetramethyl-2-(prop-1-en-1-yl)-1,3,2-dioxaborolane). The corresponding olefinic stannanes, for instance tributylvinyltin (also known as tributyl(vinyl)stannane), might also be used. This reaction is typically mediated by a palladium catalyst, such as PdCl₂; Pd(OAc)₂, or [(Ph₃P)]₄Pd, oftentimes in the presence of a ligand for Pd, such as dibenzylideneacetone or 1,1′-bis(diphenylphosphino)ferrocene (dppf). Typically, an acid scavenger, like Et₃N, Hunig's base, potassium carbonate, or cesium carbonate, is used, and the reaction is conducted in an appropriate solvent, such as ethanol, tetrahydrofuran, or 1,4-dioxane. As an example of this type of reaction, compound 3-B can be reacted with potassium vinyltrifluoroborate, PdCl₂(dppf), and Et₃N in ethanol to afford 3-C(R³═R⁴═H). Reaction of 3-C with an appropriate amine, for instance benzylamine, produces 3-D. This reaction oftentimes requires an acidic catalyst, such as acetic acid, in methanol or ethanol as the solvent. Removal of the protecting group then delivers 3-E. Conditions for adding and removing protecting groups are described in established reference volumes, for instance Greene's Protective Groups in Organic Synthesis, 4′ Edition (ISBN:9780470053485). In the case of 3-E, the 4-methoxybenzyl group can be selectively removed using trifluoroacetic acid (TFA) in a neutral solvent like CH₂Cl₂ or 1,2-dichloroethane. Compound 3-E will react with aldehydes in a classic Pictet-Spengler-type reaction. Typical solvents for this type of reaction include methanol, ethanol, n-butanol, or DMSO, and sometimes, a base like Et₃N, Hunig's base, or K₂CO₃ is used. For example, reaction of 3-E with formalin in methanol or ethanol affords the tetrahydronaphthyridine derivative 3-F. Reaction with other aldehydes, for instance acetaldehyde, would deliver 3-F (R₅═CH₃). If desired, the benzyl group on the basic nitrogen of 3-F can be removed by hydrogenolysis over a palladium catalyst, typically using hydrogen gas in the presence of a palladium catalyst (oftentimes palladium on activated carbon). This reaction is typically conducted in a solvent like acetic acid, methanol, ethanol, ethyl acetate, or mixtures thereof. Sometimes, a strong acid (like HCl) is incorporated into the reaction to afford the acid salt of the amine. Alternatively, if this reaction is conducted in the presence of Boc anhydride, in a solvent like methanol, ethanol, ethyl acetate, or mixtures thereof, the N-Boc derivative 3-G would emerge from the reaction. Compounds 3-F and 3-G are both useful intermediates for preparing the compounds of this invention.

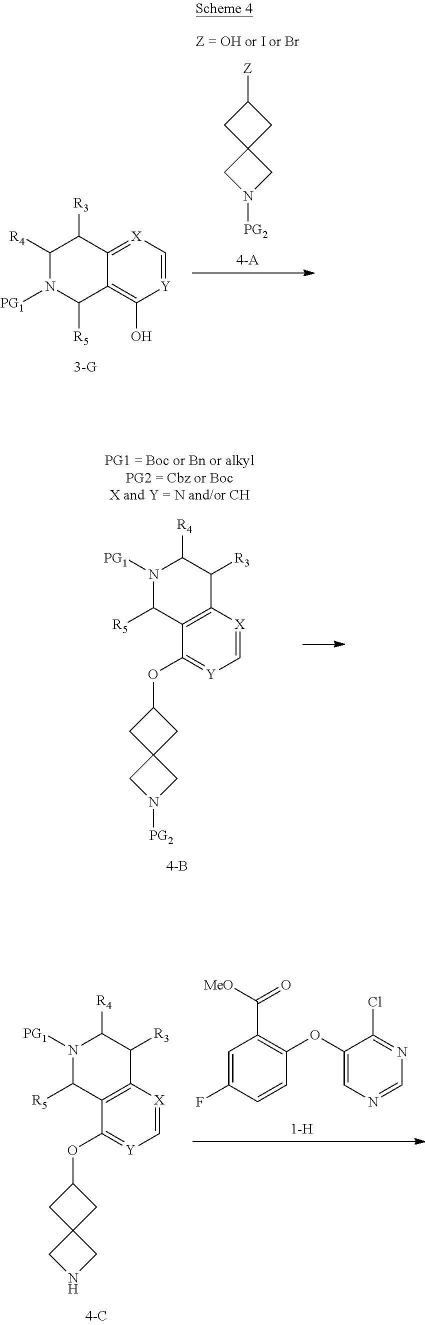

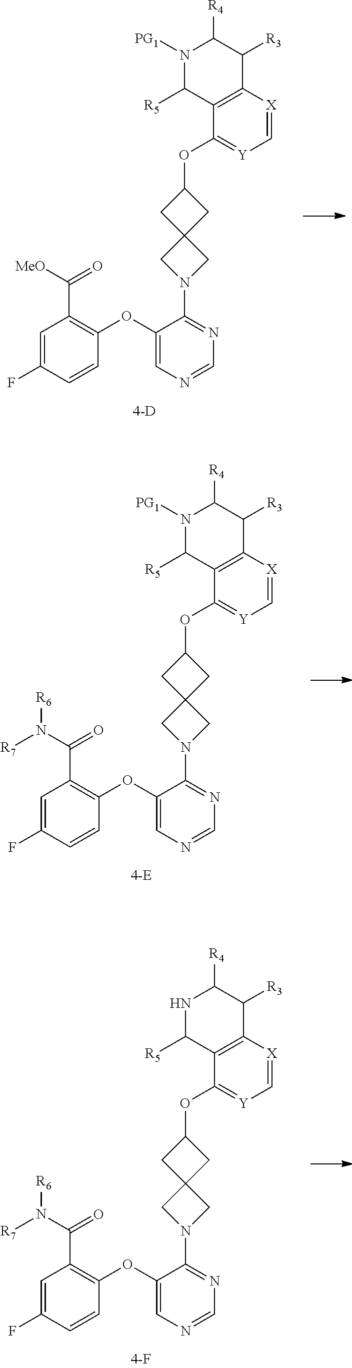

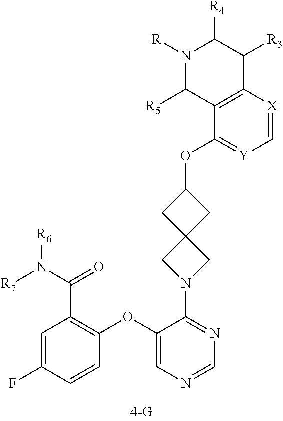

Scheme 4 illustrates the general methods for converting the key intermediate 3-G into the compounds of the present invention (4-F and 4-G) (X and Y═N and/or CH). Compound 3-G (X═N, Y═CH), which can be prepared according to the general procedures described in Scheme 3, can be reacted with an appropriate aliphatic iodo/bromo compound 4-A, under standard O-alkylation conditions, to afford compound 4-B. The O-alkylation reaction can be performed under a wide range of conditions well-known to those of skill in the art. For example, compound 3-G and benzyl 6-iodo-2-azaspiro[3.3]heptane-2-carboxylate can be combined in the presence of a base, such as Na₂CO₃, K₂CO₃, or Cs₂CO₃, in a neutral solvent like THF, DMF, DMSO or NMP, at elevated temperature, to afford 4-B. Alternatively, 4-B can be synthesized using 3-G and an appropriate alcohol, for instance, a suitably protected variant of 2-azaspiro[3.3]heptan-6-ol, under Mitsunobu conditions. Typically, a Mitsunobu reaction is conducted using a diazodicarboxylate, typically DEAD (diethyl azodicarboxylate), DIAD (diisopropyl azodicarboxylate), or ADDP (1,1′-(azodicarbonyl)dipiperidine), and an appropriate phosphine, typically PPh₃ (triphenylphosphine). Alternatively, the coupling can be mediated by the Tsunoda reagent (2-tributylphosphoranylidene)acetonitrile). The reaction is conducted in a neutral solvent like THF, ACN, CH₂Cl₂ or toluene. On the other hand, one might employ a copper (II) catalyzed coupling reaction to prepare 4-B, using an appropriate boron reagent like a potassium aryltrifluoroborate, and a suitably protected variant of 2-azaspiro[3.3]heptan-6-ol. This type of reaction is usually conducted in the presence of a ligand like TMEDA or 1,10-phenanthroline, in the presence of a base like pyridine or DMAP, in a neutral solvent like CH₂Cl₂. Another approach to prepare compound 4-B could be to react compound 3-G (where, X and Y═N, Cl instead of OH) with an appropriate alcohol 4-A, using a strong base like KOtBu or sodium hydride, in a neutral solvent like THF, DMF, DMSO or NMP. As an example of this type of reaction, compound 3-G (where X and Y═N; Cl instead of OH) can be reacted with benzyl 6-hydroxy-2-azaspiro[3.3]heptane-2-carboxylate and sodium hydride in DMF to afford 4-B (where X and Y═N) at RT. Removal of the protecting group then affords compound 4-C, which is a useful intermediate that can be used to make additional compounds. The use of protecting groups in organic synthesis is well-known to those of skill in the art, and conditions for adding and removing protecting groups are described in established reference volumes, for instance, Greene's Protective Groups in Organic Synthesis, 4^{th} Edition (ISBN:9780470053485). For instance, when the protecting group PG2 in 4-B is Cbz, deprotection is oftentimes achieved under hydrogenolysis conditions, to give compound 4-C. Hydrogenolysis is accomplished by reaction with hydrogen gas, generally in the presence of a palladium catalyst, such as palladium on activated carbon (Pd/C) or Pd(OH)₂. Sometimes, AcOH or HCl is added to the reaction mixture, and a suitable solvent, like methanol, ethanol, 2,2,2-trifluoroethanol, ethyl acetate, or a combination thereof, is typically used. Sometimes, PtO₂ can be used as a hydrogenolysis catalyst. When the protecting group PG2 in 4-B is Boc (tert-butyloxycarbonyl), deprotection is oftentimes accomplished under acidic conditions, using acids like HCl, TFA (trifluoracetic acid), or p-TsOH (para-toluenesulfonic acid), in a suitable solvent, oftentimes CH₂Cl₂, THF or 1,4-dioxane. Preferably, the Boc group in compound 4-B is removed with TMSCl in 2,2,2-trifluoroethanol.

The amine compound 4-C can be readily reacted with the chloride of compound 1-H. The ester group is subsequently saponified to the corresponding carboxylic acid (not shown in the scheme). Typically, this transformation is accomplished using LiOH, NaOH or KOH, in an aqueous solvent system, such as methanol/water or THF/water. If desired, the carboxylic acid can also be isolated as the Li, Na, or K salt. The carboxylic acid is then converted to an amide (4-E). Many different conditions have been developed to achieve this type of transformation and will be generally familiar to those of skill in the art. For instance, the carboxylic acid can be reacted with phosphorus trichloride, thionyl chloride or oxalyl chloride, which forms the corresponding acid chloride. This reaction can be conducted in a suitable, neutral solvent like CH₂Cl₂, or if thionyl chloride is used, can be run with thionyl chloride as the reactant and solvent. The acid chloride is subsequently reacted with a suitable amine, R6R7NH (R6=alkyl and R7=substituted cyclic/acyclic amine, substituted spiro and bi-cyclic amines, cyclic amide, and sulfonamide), in the presence of an appropriate acid scavenger, for example, Et₃N, (i-Pr)₂NEt, or pyridine, in a neutral solvent like CH₂Cl₂ or THF. In some instances, pyridine can be used as the acid scavenger and the solvent for the reaction. Alternatively, the carboxylic acid and amine R⁶R⁷NH (R⁶=alkyl and R⁷=substituted cyclic/acyclic amine, substituted spiro and bi-cyclic amines, cyclic amide, and sulfonamide) can be combined and coupled using a reagent such as DCC (dicyclohexyl carbodiimide), EDC (1-ethyl-3-(4-dimethylaminopropyl)carbodiimide), HATU (1-[bis(dimethylamino)methylene]-1H-1,2,3-triazolo[4,5-b]pyridinium 3-oxide hexafluorophosphate), HBTU (2-(1H-benzotriazol-1-yl)-1,1,3,3-tetramethyluronium hexafluorophosphate), or T3P (propanephosphonic acid anhydride, 50% in EtOAc), in the presence of an appropriate acid scavenger, for example, Et₃N, (i-Pr)₂NEt, or pyridine, in a neutral solvent, oftentimes DMF, NMP, CH₂Cl₂ or THF. In some instances, when DCC or EDC is used as the coupling reagent, HOBt (hydroxybenzotriazole) might be added to the reaction to facilitate the desired coupling reaction. In some cases, phosphorus trichloride in combination with DIPEA in a neutral solvent like CH₂Cl₂. can be used for this transformation. When a protecting group (PG1) is required, it can be removed subsequently according to the nature of the protecting group. For instance, when the protecting group PG1 in 4-E is Boc, deprotection is achieved according to the general methods described for the Boc-deprotection under acidic condition (see 4-B to 4-C), to give compound 4-F. Compound 4-F can be isolated as a salt, where n HX denotes the salt form, or as the free base, following work-up under standard basic conditions. When PG1=Bn in 4-E, deprotection can be accomplished according to the general methods described for the Cbz-deprotection under hydrogenolysis conditions (see 4-B to 4-C).

If desired, 4-F can be N-alkylated to afford compound 4-G (X and Y═N and/or CH, R=an appropriate alkyl group). In this type of reaction, 4-F reacts with an alkyl halide or an alkyl sulfonate (for instance, a triflate, mesylate, or tosylate) to afford the alkylated product 5-G. This reaction can be conducted in the presence of a base like, Na₂CO₃, K₂CO₃, Cs₂CO₃, Et₃N (triethylamine) or (i-Pr)₂NEt (diisopropylethylamine, also known as Hunig's base), in a suitable, neutral solvent, such as ACN, THF, DMF, DMA (dimethyl acetamide) or NMP (N-methylpyrrolidinone). Alternatively, 4-F can react with a suitable aldehyde or ketone in a reductive amination reaction. This reaction can be conducted in the presence of STAB (sodium triacetoxyborohydride) or NaCNBH₃ (sodium cyanoborohydride) and acetic acid, in a suitable solvent, such as MeOH, DCE, CH₂Cl₂, THF, DMF or NMP or a combination thereof. In a representative example, formaldehyde can be reacted with 4-F in the presence of STAB (sodium triacetoxyborohydride) and AcOH, in DCE to afford the N-methylated compound 4-G. Alternatively, another approach to convert compound 4-F to 4-G is to react 4-F with a dihaloalkane (like 1-bromo-3-chloropropane) in the presence of Na₂CO₃, K₂CO₃, Cs₂CO₃, Et₃N or (i-Pr)₂NEt, in a suitable, neutral solvent, such as ACN, THF, DMF or DMA to obtain the corresponding N-alkylated halo intermediate (not shown in the scheme). This can further react with another amine R₆R₇NH, in the presence of a base, such as Et₃N or (i-Pr)₂NEt), in a suitable, neutral solvent, such as ACN, THF or DMF, to afford compound 4-G (wherein R contains the R₆R₇N— group).

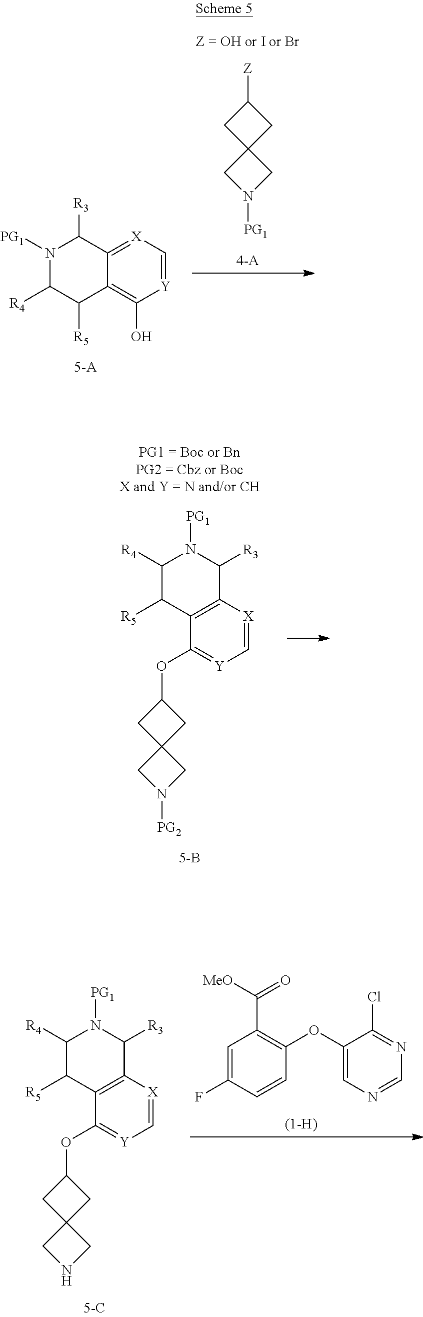

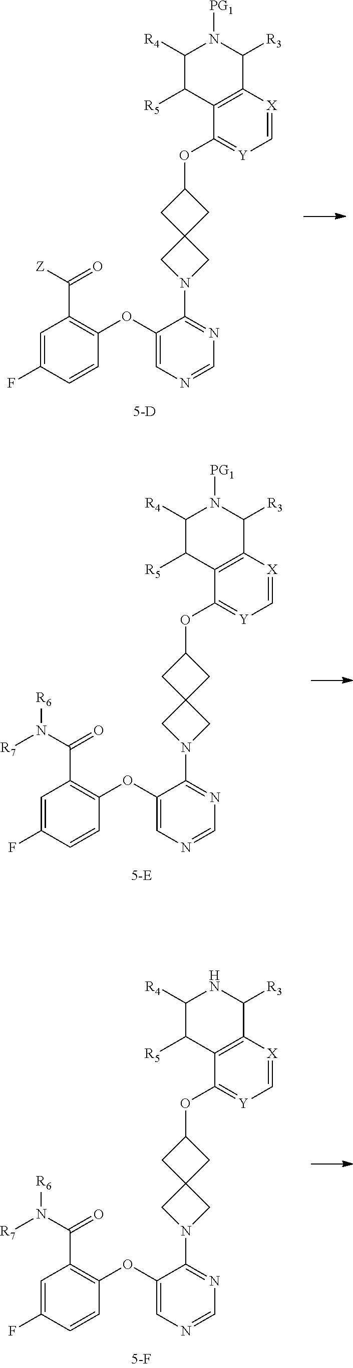

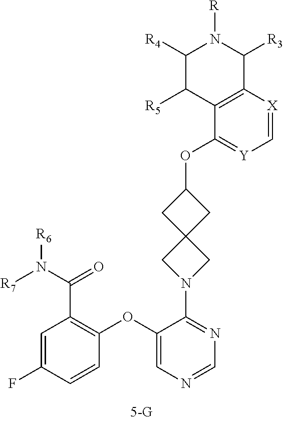

Scheme 5 illustrates the general methods for converting compound 5-A into the compounds of the present invention (5-F and 5-G) (X and Y═N and/or CH). O-Alkylation of 5-A using 4-A can be accomplished using the general procedures described in Scheme 4 (see 3-G to 4-B). Removal of the protecting group (PG2=Cbz or Boc) of 5-B can be accomplished using the general procedures for removing Cbz or Boc group as described in Scheme 4 (see 4-B to 4-C) to afford 5-C. This can be converted to the compound (5-E) as described in Scheme 4 (see 4-C to 4-E). Removal of the protecting group (PG1=Boc or Bn) of 5-E affords 5-F (see 4-B to 4-C). Subsequently, if desired, N-alkylation with a suitable alkylating agent, according to the general methods described in Scheme 4 (see 4-F to 4-G), affords 5-G. The resulting amines 5-F and 5-G can be isolated as the free base or as a suitable salt form, as discussed in Scheme 4.

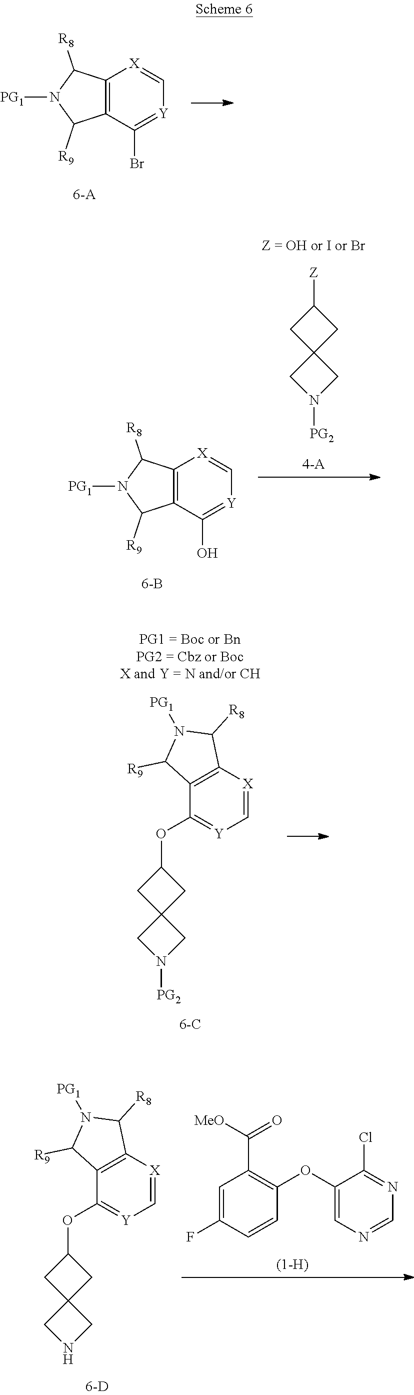

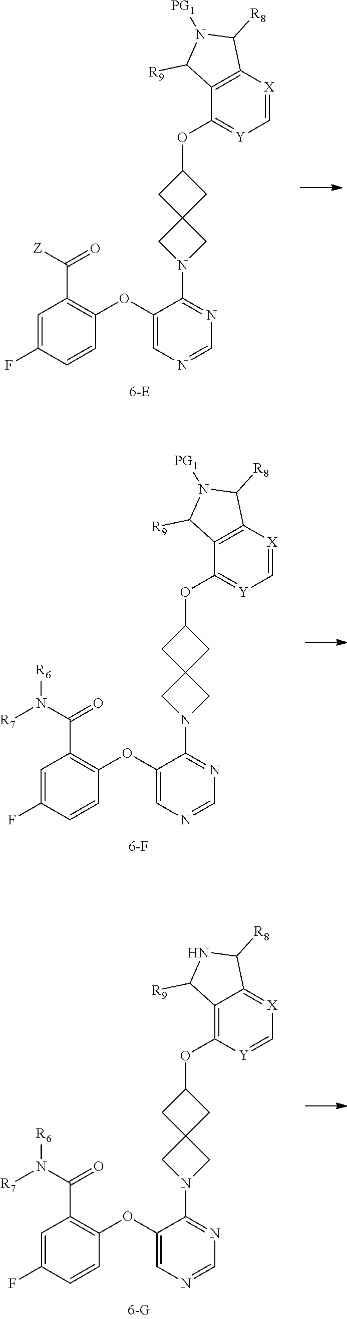

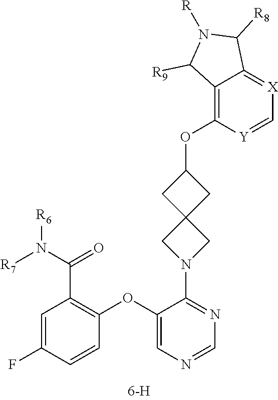

Scheme 6 illustrates the general methods for converting compound 6-A into the compounds of the present invention (6-G and 6-H) (X and Y═N and/or CH). Hydroxylation of 6-A can be accomplished using KOH or NaOH in the presence of Pd catalyst, such as PdCl₂; Pd(OAc)₂, or [(Ph₃P)]₄Pd, oftentimes in the presence of a ligand for Pd, such as dibenzylideneacetone or 1,1′-bis(diphenylphosphino)ferrocene (dppf). Solvents for this type of reaction typically include THF, DMSO, or toluene with water. For instance, 6-A can be converted to 4-hydroxy azaisoindoline 6-B using KOH in the presence of Pd₂(dba)₃ and tBuXphos in 1,4-dioxane and water. O-Alkylation of 6-B with 4-A can be accomplished using the general procedures described in Scheme 4 (see 3-G to 4-B). The protecting group (PG2=Cbz or Boc) of 6-C can be removed using the general procedures for removing Cbz or Boc as described in Scheme 4 (see 4-B to 4-C) to afford 6-D. Compound 6-D can then be converted to 6-F as described in Scheme 4 (see 4-C to 4-E). Removal of the protecting group (PG1=Boc or Bn) of 6-F affords 6-G (see 4-B to 4-C). Subsequent N-alkylation, as discussed in Scheme 4 (see 4-F to 4-G), affords 6-H. The resulting amines 6-G and 6-H can be isolated as the free base or as a suitable salt form, as is discussed in Scheme 4.

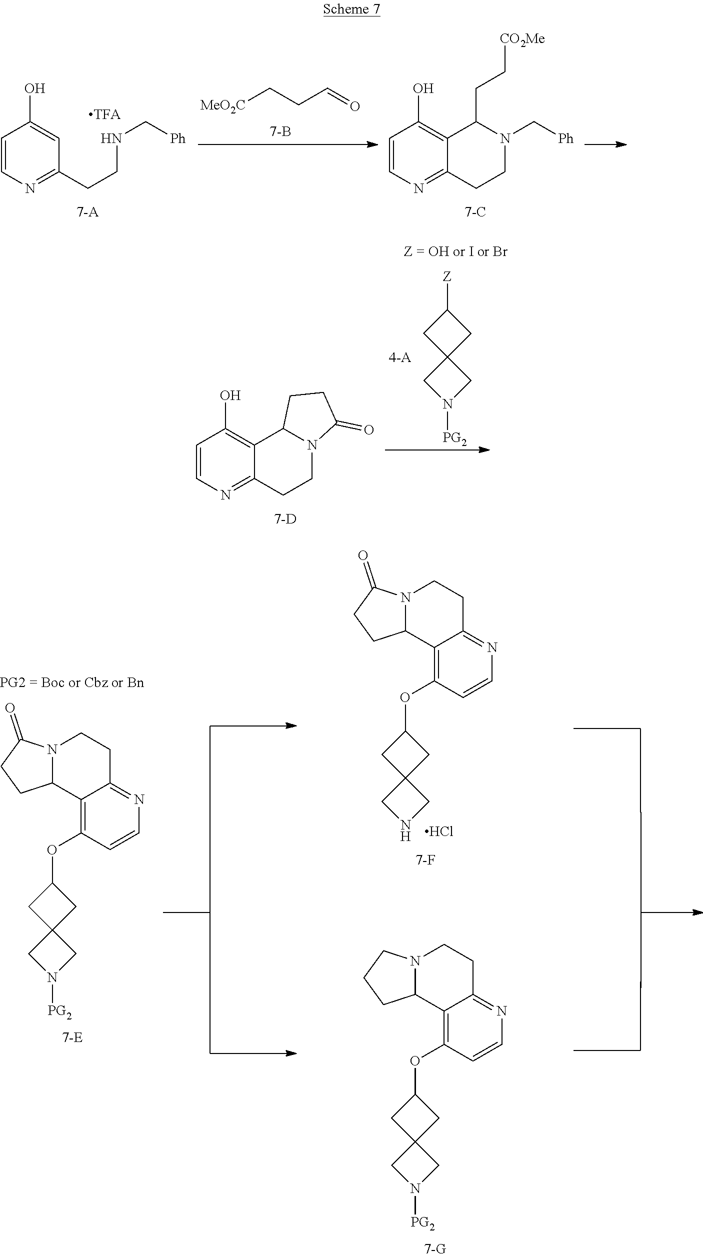

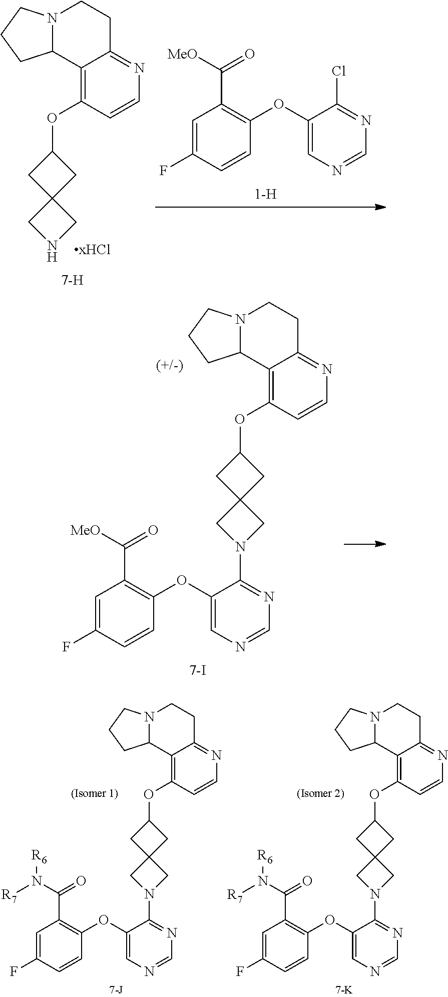

Scheme 7 illustrates the general methods for converting compound 7-A into compounds 7-J and 7-K. Compound 7-A can be reacted with aldehydes in a Pictet-Spengler-type reaction, according to the general procedures described in Scheme 3 (see 3-E to 3-F). For example, reaction of 7-A with methyl 4-oxobutanoate in DMSO affords the tetrahydronaphthyridine derivative 7-C. The benzyl group on the basic nitrogen of 5-C can be removed by hydrogenolysis, typically using hydrogen gas with a palladium catalyst (oftentimes palladium on activated carbon), in the presence of catalytic acetic acid, in MeOH or ethanol as solvent. The resulting free amine (not shown in the scheme) typically reacts with the ester in situ to afford the lactam product 7-D. Compound 7-D can be O-alkylated using a suitable alkylating reagent, following the general methods described in Scheme 4 (see 3-G to 4-B). Preferably, 7-D can be reacted with 4-A (tert-butyl 6-iodo-2-azaspiro[3.3]heptane-2-carboxylate) under basic conditions, such as K₂CO₃, in a neutral solvent, such as DMF, at elevated temperature, to afford 7-E. The protecting group (Boc or Cbz or Bn) of 7-E can be removed using the general procedures for removing a Boc or Cbz or Bn protecting group described in Scheme 4 (see 4-B to 4-C), to afford 7-F. The amide group of 7-F can be subsequently reduced to the corresponding amine 7-H. Typically, this transformation is accomplished using aluminum hydride or boron hydride reducing agents (for example, LiAlH₄, BH₃·Et₂O, BH₃·DMS, or 9-BBN), in neutral solvents such as THF, Et₂O, DCM, hexane or toluene. As an example, the amide group of 7-F can be reduced to the amine 7-H using LiAlH₄ in THF. Alternatively, the amide group of 7-E can be reduced and then the protecting group (Boc or Cbz or Bn) of 7-G can be removed to afford 7-H. Another method for reduction of the lactam 7-E to the corresponding amine 7-G is to use transition-metal catalysis in the presence of an appropriate reducing agent. Typically, this type of reaction is conducted in the presence of transition metal complexes of rhodium [RhH(CO)(PPh₃)₃], manganese [CpMn(CO)₃], iron [Fe(CO)₅ or Fe₃(CO)₁₂ or [Fe₃(CO)₁₁(μ-H)]₂Fe(DMF)₄], cobalt [Co₂(CO)₈] or nickel [Ni(OAc)₂ or NiCl₂(DME)], using hydrosilanes [PhSiH₃ or Ph₂SiH₂ or =polymethylhydrosiloxane (PMHS) or tetramethyldisiloxane (TMDS)], hydroboranes [HBPin or HBCat (Pin=pinacol, Cat=catechol)], or H₂ as the reducing agent. The reaction is typically conducted in THF or toluene. In a representative example, 7-E was converted to corresponding amine 7-G using [RhH(CO)(PPh₃)₃] and PhSiH₃ in THF. Many other methods can be employed to reduce the amide to the corresponding amine. For example, the amide can be activated using Tf₂O (trifluoromethanesulfonic anhydride, trifluoromethanesulfonic acid), or strong Lewis acids, such as such as Yb(OTf)₃ or BF₃, or B(C₆F₅)₃ or BF₃·Et₂O, followed by either hydrogenation using a transition metal catalyst and H₂ or reduction with a reducing agent like NaBH₄ or tetramethyldisiloxane. The protecting group (Boc or Cbz or Bn) of 7-G can be removed using the general procedures as previously described in Scheme 4. The amine 7-H can be isolated as the free base or as a suitable salt form, as discussed in Scheme 4. Compound 7-H is then converted to 7-I according to the general methods described previously (see earlier Scheme 4). The ester group of 7-1 is subsequently saponified to the corresponding carboxylic acid (not shown). The carboxylic acid is then converted to the racemate corresponding to 7-J and 7-K, according to the general methods for amide formation described in Scheme 4. In a representative example, the carboxylic acid is reacted with a suitable amine, R6R7NH (R6=alkyl and R7=substituted cyclic/acyclic amine, substituted spiro and bi-cyclic amines, cyclic amide, and sulfonamide), using HATU as the coupling reagent, in the presence of (i-Pr)₂NEt as the acid scavenger, in CH₂Cl₂ as solvent, to obtain the racemate corresponding to 7-L and 7-K (not shown). The pure enantiomers, 7-J and 7-K, can be isolated by chiral purification using HPLC or SFC. Typically, this purification is accomplished using a chiral SFC method.

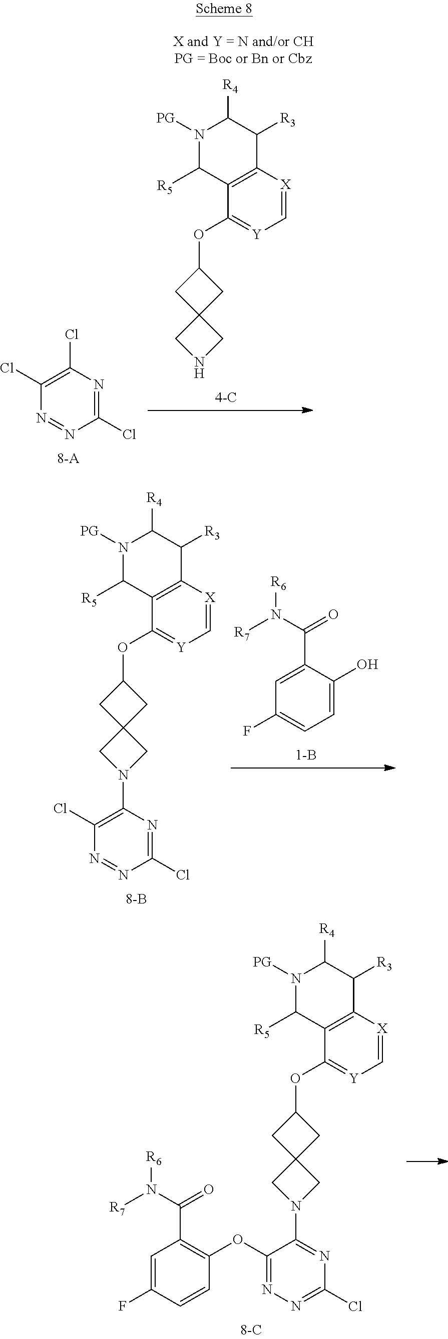

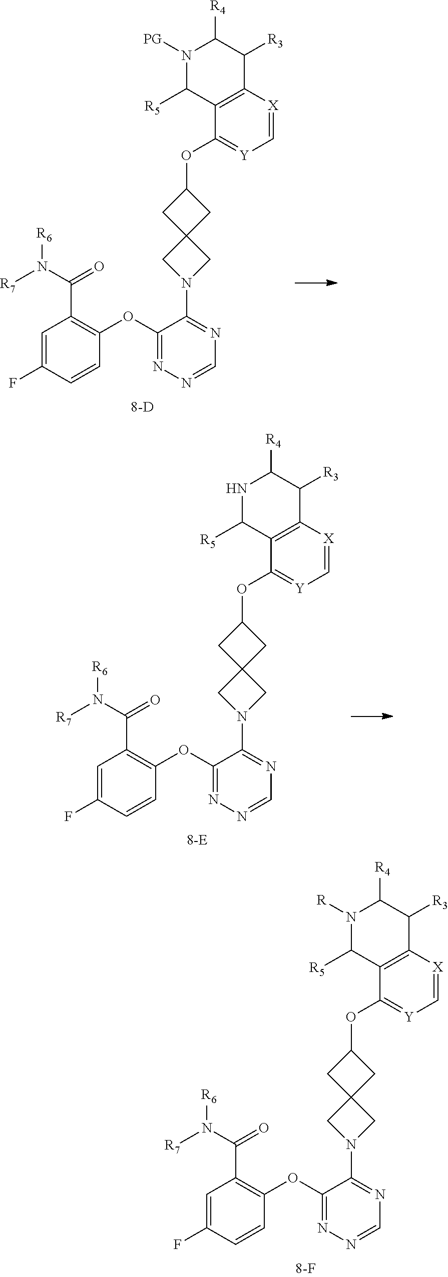

Scheme 8 illustrates the general methods for converting compound 4-C into 8-E and 8-F (X and Y═N and/or CH). Under suitable conditions, 3,5,6-trichloro-1,2,4-triazine (8-A) can be reacted with various amines to afford the product resulting from the displacement of the chloro-group at the 5-position. For example, compound 8-A can be reacted with tert-butyl 4-((2-azaspiro[3.3]heptan-6-yl)oxy)-7,8-dihydro-1,6-naphthyridine-6(5H)-carboxylate to afford compound 8-B. Typically, this reaction is conducted in the presence of a suitable base, such as Et₃N or (i-Pr)₂NEt, and in a neutral solvent like CH₂Cl₂, THF or NMP. Under appropriate conditions, phenol 1-B can react with compound 8-B to afford compound 8-C, via displacement of the chloro-group at the 6-position of 8-B. This reaction is conducted in the presence of a suitable base, oftentimes DBU (1,8-diazabicyclo[5.4.0]undec-7-ene) or tetramethyl guanidine, in a neutral solvent, such as THF, 2-MeTHF, or CPME (cyclopentyl methyl ether). Many different acid scavengers, for example, Et₃N, (i-Pr)₂NEt, or pyridine, in a neutral solvent like CH₂Cl₂ or dioxane, can be used to achieve this type of transformation. Those of skill in the art will recognize that competing displacement of the chloro-group at the 3-position might also occur, to varying degrees, during this reaction. As desired, the chloro-group at the 3-position of compound 8-C can be retained, displaced by a nucleophile, or removed to afford compound 8-D. Removal of the chloro group and the protecting group (where PG=benzyl) of 8-C is typically accomplished in one pot under reductive conditions to afford compounds 8-E. This transformation is oftentimes achieved by reaction with hydrogen gas in the presence of a catalyst like Pd—C, Pd(OH)₂/C, Raney Nickel, Ru/C or Ir/C, in a suitable solvent, usually methanol, ethanol, 2,2,2-trifluoroethanol, ethyl acetate, or a combination thereof. When PG=Boc, hydrogenation will remove the chloro group but leave the Boc protecting group. The dechlorination reaction can also be accomplished using a metal hydride like NaBH₄, LiBH₄ or 9-BBN, generally in the presence of a palladium catalyst, like Pd(dppf)Cl₂ or Pd(OAc)₂. Removal of the Boc group of 8-D to affords 8-E, and subsequent N-alkylation, as illustrated in Scheme 4 (see 4-F to 4-G), affords 8-F (R=an appropriate alkyl group). The resulting amines 8-E and 8-F can be isolated as the free base or as a suitable salt form, as is discussed in Scheme 4.

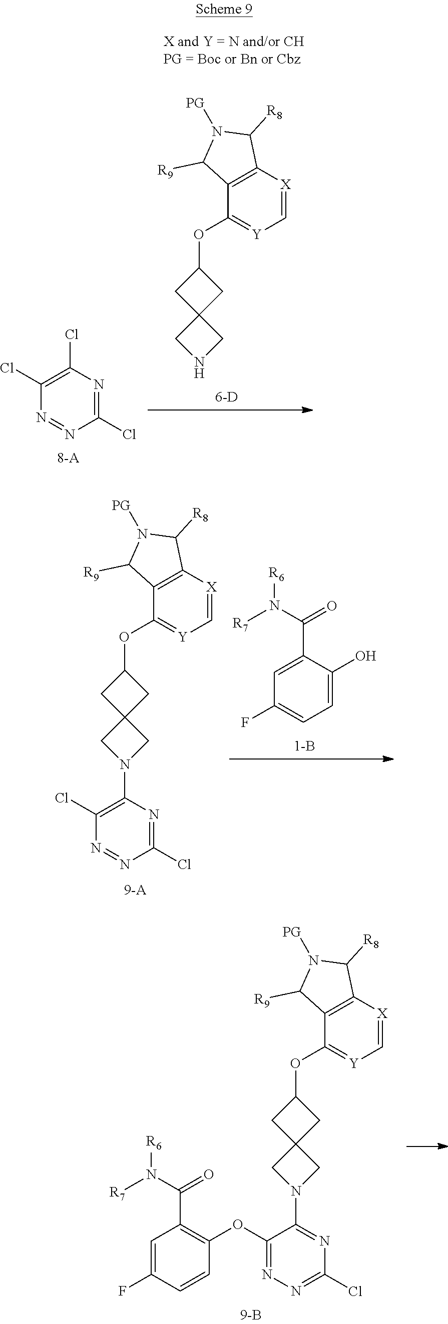

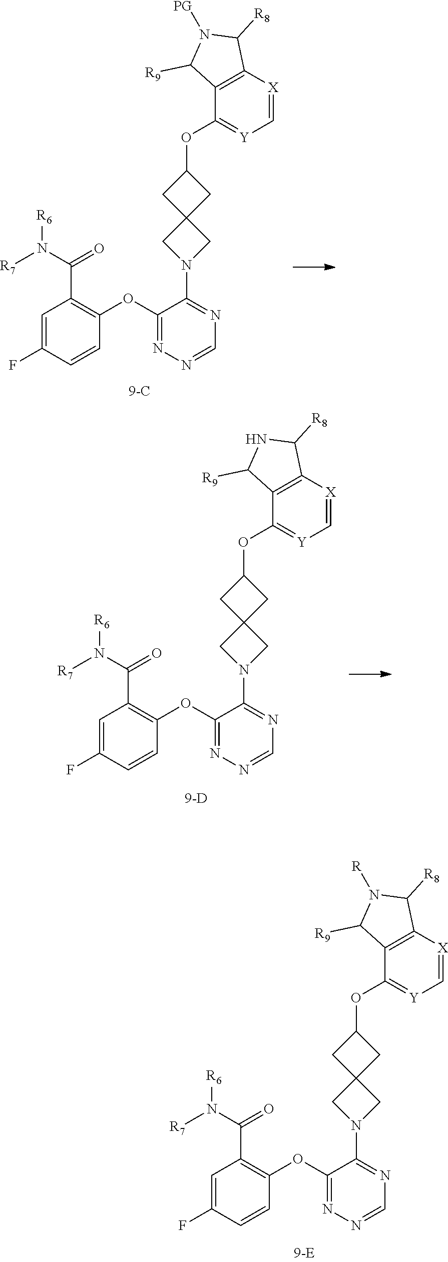

Scheme 9 illustrates the general methods for converting compound 6-D into 9-D and 9-E (X and Y═N and/or CH). Chloro displacement at the 5-position with various amines 6-D to afford the product 9-A can be accomplished using the general procedures described in Scheme 8 (see 8-A to B—B). Subsequently, as described in Scheme 8 (see B—B to 8-C), phenol 1-B can react with compound 9-A to afford compound 9-B, Wia displacement of the chloro-group at the 6-position of 9-A. Removal of the chloro-group and the protecting group (where PG=benzyl or Boc) of 9-B can be accomplished as illustrated in Scheme 8 (8-C to 8-E), to afford compounds 9-D. Subsequent N-alkylation using a range of alkylating agents, as described in Scheme 4 (see 4-F to 4-G), affords compounds 9-E (R=an appropriate alkyl group). The resulting amines 9-D and 9-E can be isolated as the free base or as a suitable salt form, as is discussed in Scheme 4.

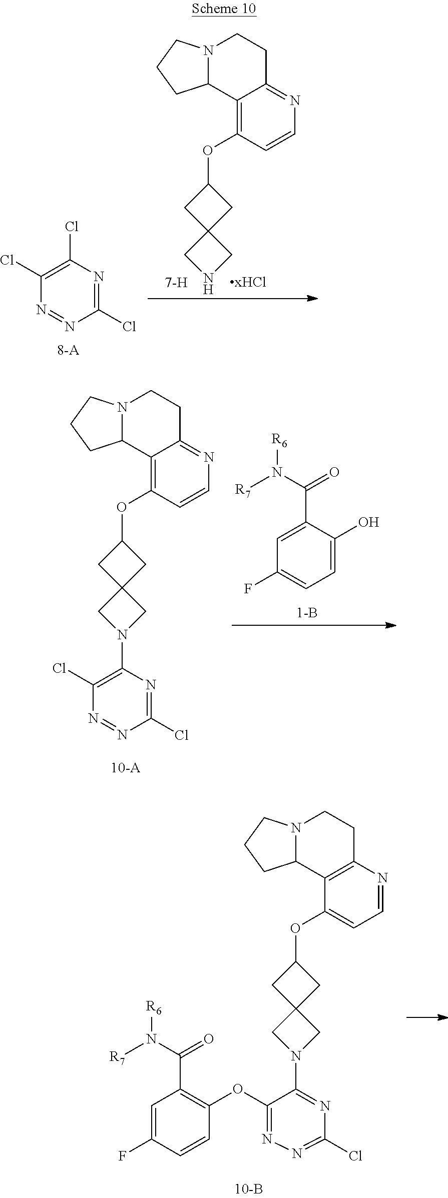

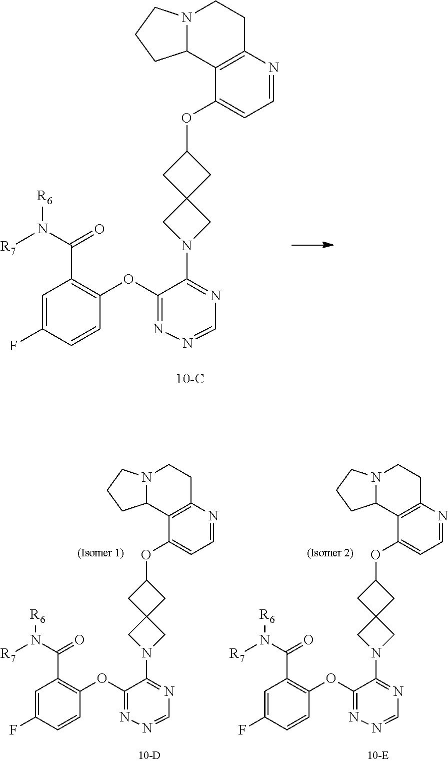

Scheme 10 illustrates the general methods for converting compound 7-H into 10-D and 10-E. Chloro displacement at the 5-position with various amines 7-H to afford the product 10-A can be accomplished using the general procedures described in Scheme 8 (see 8-A to 8-B). Under the general conditions described in Scheme 8 (see 8-B to 8-C), phenol 1-B can react with compound 10-A to afford compound 10-B, via displacement of the chloro-group at the 6-position of 10-A. Removal of the chloro-group of 10-B can be accomplished as illustrated in Scheme 8 (8-C to 8-E), to afford compounds racemic amine 10-C. The pure enantiomers, 10-D and 10-E can be isolated by chiral purification using HPLC or SFC. Typically, this purification is accomplished using a chiral SFC method.

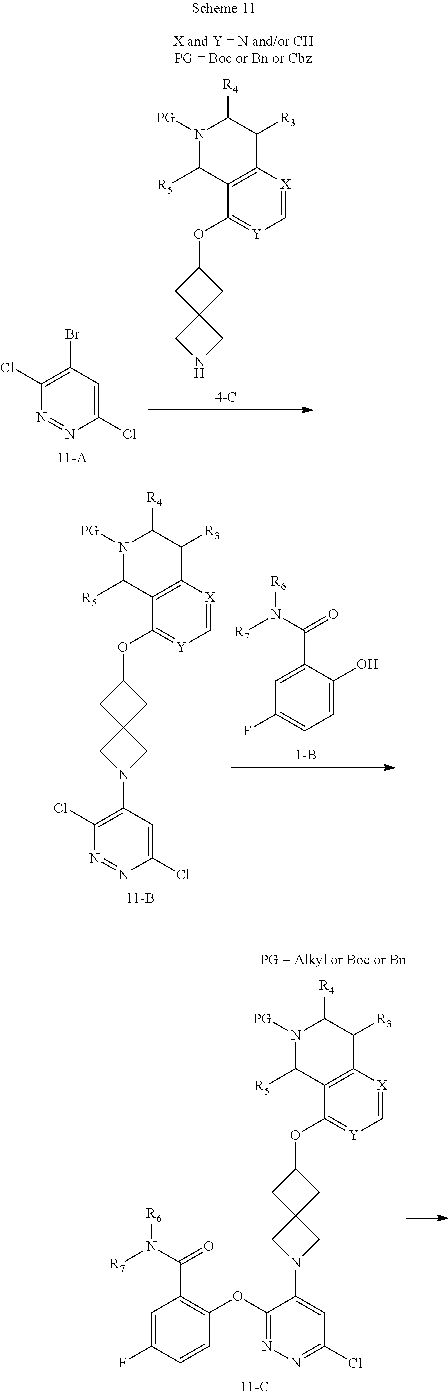

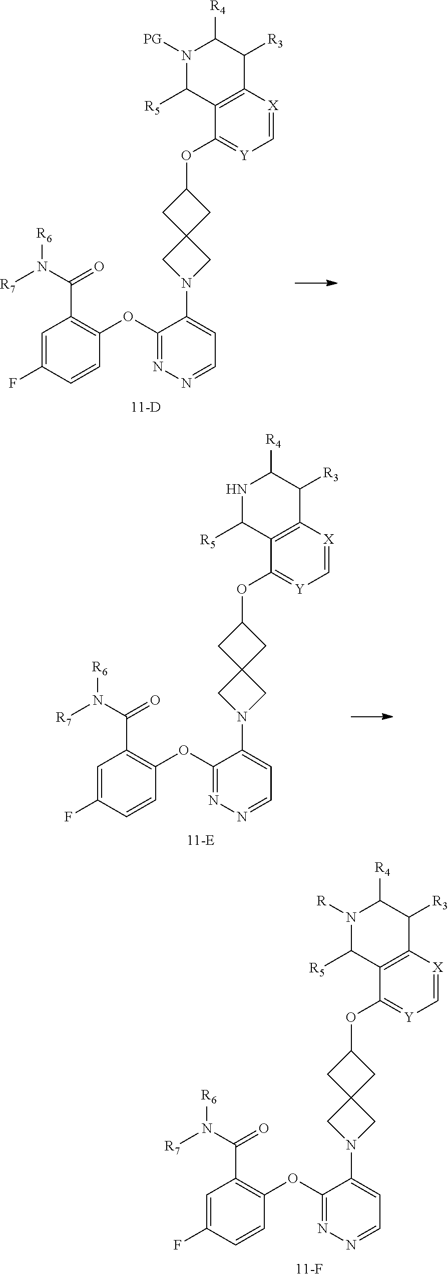

Scheme 11 illustrates the general methods for converting compound 4-C into compounds 11-E and 11-F (X and Y═N and/or CH). According to the general procedures described in Scheme 8 (see 8-A to 8-B), displacement of the bromo group at the 4-position of 4-bromo-3,6-dichloropyridazine (compound 11-A) with various amines 4-C affords compound 11-B. As an example of this transformation, 11-A is reacted with tert-butyl 4-((2-azaspiro[3.3]heptan-6-yl)oxy)-7,8-dihydro-1,6-naphthyridine-6(5H)-carboxylate in the presence of K₂CO₃ in DME to afford 11-B. Subsequently, under the general conditions described in Scheme 8 (see 8-B to 8-C), 4-((2-(3,6-dichloropyridazin-4-yl)-2-azaspiro[3.3]heptan-6-yl)oxy)-6-methyl-5,6,7,8-tetrahydro-1,6-naphthyridine 11-B is reacted with N-((1r,3r)-3-(dimethylamino)-3-methylcyclobutyl)-5-fluoro-2-hydroxy-N-isopropylbenzamide 1-B in the presence of a suitable base, K₂CO₃, in 1,4-dioxane, under elevated temperature to afford corresponding compound 11-C. Removal of the chloro group and the protecting group (where PG=benzyl) of 11-C is typically accomplished in one pot under reductive conditions, as generally described previously, to afford compounds 11-E. Removal of the Boc group of 11-D to afford compounds 11-E is accomplished as described in Scheme 4 (see 4-E to 4-G). Subsequent N-alkylation can be accomplished using a wide range of appropriate alkylating agents, as illustrated in Scheme 4 (see 4-F to 4-G), to afford the compounds 11-F (R=an appropriate alkyl group). The resulting amine 11-E and 11-F can be isolated as the free base or as a suitable salt form, as is discussed in Scheme 4.

### General Biological Methods

### Menin-MLL Competition and MV4; Cell Proliferation Assays

Menin-MLL is a competition assay between human Menin and N-terminal portion of human MLL representing amino acids 4-43 of the protein. The interaction between Menin and MLL peptide was monitored by HTRF employing Terbium labeled anti-His6 antibody directed to the N-terminal His6-tag on recombinant Menin and FITC group covalently attached to the MLL peptide. The N-terminal fragment of MLL, retained in all MLL fusion proteins, is involved in the interactions with Menin, and this protein-protein interaction is critical for the MLL fusion proteins mediated leukemogenic transformations.

For IC₅₀ determination test compounds may be prepared as stock solutions. Lower sub-stocks of 50 μM may be prepared from the 10 mM stock solution. To test the compounds in assay, 3.16-fold serial dilutions are made in 100% DMSO. Mid-stock of 50× compounds (50 μM) were serially diluted (3.16 fold) in 100% DMSO in Polypropylene plate. In assay plate 1 micro-litre of the previously prepared compound dilution was stamped. H-FL-Menin diluted to 4 nM in assay buffer (50 mM Tris-HCl, pH 7.4, 50 mM NaCl, freshly prepared 1 mM DTT, 0.01% BSA, 0.005% Triton X-100) was pre-incubated with 8 nM anti-His6-Tb for 30 min at room temperature. FITC-MLL-4-43 was diluted to 2 nM in assay buffer and 25 μL was dispensed into each well of the assay plate followed by addition of 25 μl of pre-incubated H-FL-Menin and anti-His6-Tb mixture. Final concentration H-FL-Menin diluted to 1 nM in assay plate with 2 nM anti-His6-Tb and 1 nM FITC-MLL-4-43. After 1 hr incubation at room temperature, the HTRF signal was measured on the Spark multi-label plate reader. Resulting data were captured as a ratio of RFU520/RFU485×1000. The max values were obtained from 0% inhibition in presence of 2% DMSO and the min. values were 100% inhibition in presence of 1 μM reference compound.

### Cell Proliferation Assay—MV4; 11

Compounds were evaluated for its capacity to inhibit the proliferation of the MLLr leukemia cell line MV4-11 that harbors an MLL1-AF4 fusion protein. MV4-11 cells were cultured for 72 hours with limiting dilutions of compounds and viability was measured using CellTiter-Glo.

Compounds were dissolved to obtain as 10 mM solution in DMSO. The stock was diluted 1:5 to the top concentration of 2 mM in 100% DMSO. For IC₅₀ determination, serial 1:3.16 dilutions were prepared in 100% DMSO by diluting 20 μL into 43.5 μL of DMSO for 8 concentrations. Each prepared DMSO solution were further diluted 1:500 in the cell culture media to obtain the 2× dosing solutions. The final concentrations of tested compounds in the cell culture media ranged from 0.632 nM to 2000 nM.

MV4-11 cells were cultured in IMDM with 10% FBS and 1× Penicillin-Streptomycin at 5% CO₂ and 37° C. A cell suspension was prepared containing 15,000 cells/ml in the culture medium and 100 μL of this suspension was added per well to a 96-well cell culture plate. Then, 100 μL of 2× dosing media containing test compounds were added bringing the total volume to 200 μL. These cells were cultured for 72 hours at 37° C. and 5% CO₂ in a humidified incubator.

After 72 hours, the cultured cells were mixed and 100 μL was transferred to a 96-well black plate. Cell Titer Glo (100 μl) was then added to this plate. The plate was mixed with shaking for 15 mins at RT, the luminescence was then measured using Tecan Spark 20 M spectrophotometer. Cell Viability (%) was determined by RLU of test/RLU average vehicle control*100 and % max inhibition was determined by 100−(% cell viability remaining at the top concentration of compound).

hERG Patch Clamp Assay

The hERG inhibition assay uses a high throughput single cell planar patch clamp approach. Chinese hamster ovary cells transfected with the hERG gene (CHO-hERG) are dispensed into the PatchPlate. Amphotericin is used as a perforating agent to gain electrical access to the cells. The hERG tail current is measured prior to the addition of the test compound by perforated patch clamping. Following addition of the test compound at a defined concentration or range of concentrations a second recording of the hERG current is performed. The degree of inhibition (%) is obtained by measuring the tail current amplitude, which is induced by a one second test pulse to −40 mV after a two second pulse to +20 mV, before and after drug incubation (the difference current is normalized to control and multiplied by 100 to obtain the percent of inhibition). The patch clamp assay can be used according to the knowledge of a person of ordinary skill in the art to accordingly assess the compounds of the present disclosure.

In any one of the embodiments described herein, the compound has an IC₅₀ of more than 10, 15, 20, 25, or 30 μM in a standard human ether-a-go-go related gene (hERG) patch clamp assay.

A number of drugs have been withdrawn from late-stage clinical trials due to cardiotoxic effects, therefore it is important to identify and avoid compounds with potential for cardiotoxic effects early in drug discovery. The cardiovascular toxicity of a compound can be measured using a standard human ether-a-go-go related gene (hERG) assay. The human ether-a-go-go related gene (hERG) encodes the inward rectifying voltage gated potassium channel in the heart (IKr), which is involved in cardiac repolarization. Inhibition of the hERG current causes QT interval prolongation resulting in potentially fatal ventricular tachyarrhythmia called Torsade de Pointes. A compound having an IC50 of more than about 10 μM or more than about 15 μM, in the hERG assay may be considered as free from any cardiovascular toxicity. In some embodiments, the compounds of Formulae I′, I, I-a, I-b, and I-c, and/or Table 1 have reduced hERG binding compared to structural analogs. In some embodiments, the compounds of Formulae I′, I, I-a, 1-b, and I-c, and/or Table 1 have IC₅₀ of more than 10 μM, 15 μM, 20 μM, 25 μM, or 30 μM in the standard patch clamp hERG assay.

In some embodiments, the compounds of the present disclosure (e.g., Formulae I′, I, I-a, I-b, and I-c, and Table 1) do not significantly block the hERG potassium channel (e.g., an IC₅₀ greater than 1 μM, 5 μM, 10 μM, 15 μM, 20 μM, 25 μM, 30 μM, 35 μM, 40 μM, or 50 μM) in the standard patch clamp hERG assay.

### Methods of Use

The compounds of the invention are inhibitors of the interaction of menin with MLL and MLL fusion proteins. In some embodiments, the present disclosure is directed to a method of inhibiting the interaction between menin and MLL or an MLL fusion protein by contacting menin and MLL or the MLL fusion protein with a compound of the disclosure. The contacting can be carried out in vitro or in vivo. In some embodiments, the compounds of the disclosure can bind to menin, thereby interfering with the binding of MLL to menin. In some embodiments, the present disclosure provides a method of inhibiting the activity of menin by contacting menin with a compound of the disclosure in the presence of MLL or an MLL fusion protein. In further embodiments, the present disclosure provides a method of inhibiting the binding of MILL or an MLL fusion protein to menin, comprising contacting menin with a compound of the disclosure in the presence of the MLL or MLL fusion protein.

In some embodiments, compounds of the present disclosure minimize hERG interactions. In some embodiments, the present disclosure is directed to a method of inhibiting the interaction between menin and MILL or an MILL fusion protein by contacting menin and MLL or the MLL fusion protein with a compound of the disclosure while the compounds of the disclosure minimize hERG activity. In some embodiments, the present disclosure is directed to a method of inhibiting the interaction between menin and MLL or an MLL fusion protein by contacting menin and MLL or the MLL fusion protein with a compound of the disclosure while the compound of the disclosure avoids drug-induced blockade of hERG.

Evaluating the hERG activity can be accomplished by many methods known in the art. Including, such methods for the assessment of hERG liability is the patch-clamp electrophysiological assay on hERG transfected cells. Various other strategies including radiolabeled binding assays, functional assays, and rubidium efflux assays also quantify hERG potency.

The compounds of the disclosure are also useful in treating diseases associated with the menin-MLL interaction or menin-MLL fusion protein interaction. For example, diseases and conditions treatable according to the methods of the disclosure include cancer, such as leukemia, and other diseases or disorders mediated by the menin-MLL interaction or menin-MLL fusion protein interaction such as diabetes.

Accordingly, the compounds of the disclosure are believed to be effective against a broad range of cancers, including, but not limited to, hematological cancer (e.g., leukemia and lymphoma), bladder cancer, brain cancer (e.g., glioma, diffuse intrinsic pontine glioma (DIPG)), breast cancer (e.g., triple-negative breast cancer, estrogen-receptor-positive breast cancer (i.e., ER+ breast cancer)), colorectal cancer, cervical cancer, gastrointestinal cancer (e.g., colorectal carcinoma, gastric cancer), genitourinary cancer, head and neck cancer, liver cancer, lung cancer, melanoma, ovarian cancer, pancreatic cancer, prostate cancer (e.g., castration resistant prostate cancer), renal cancer (e.g., renal cell carcinoma), skin cancer, thyroid cancer (e.g., papillary thyroid carcinoma), testicular cancer, sarcoma (e.g., Ewing's sarcoma), and AIDS-related cancers. In some embodiments, the cancer is associated with a rearranged MLL gene. In some embodiments, the pathophysiology of the cancer is dependent on the MLL gene. In some embodiments, the cancer is associated with mutant p53 gain-of-function.

In some embodiments, the specific cancers that may be treated by the compounds, compositions and methods described herein include cardiac cancers, such as for example, sarcoma (e.g., angiosarcoma, fibrosarcoma, rhabdomyosarcoma, and liposarcoma), myxoma, rhabdomyoma, fibroma, lipoma and teratoma; lung cancers, including, for example, bronchogenic carcinoma (e.g., squamous cell, undifferentiated small cell, undifferentiated large cell, and adenocarcinoma), alveolar and bronchiolar carcinoma, bronchial adenoma, sarcoma, lymphoma, chondromatous hamartoma, mesothelioma, non-small cell lung cancer, small cell lung cancer, bronchial adenomas/carcinoids, and pleuropulmonary blastoma; gastrointestinal cancer, including, for example, cancers of the esophagus (e.g., squamous cell carcinoma, adenocarcinoma, leiomyosarcoma, and lymphoma), cancers of the stomach (e.g., carcinoma, lymphoma, and leiomyosarcoma), cancers of the pancreas (e.g., ductal adenocarcinoma, insulinoma, glucagonoma, gastrinoma, carcinoid tumors, and vipoma), cancers of the small bowel (e.g., adenocarcinoma, lymphoma, carcinoid tumors, Kaposi's sarcoma, leiomyoma, hemangioma, lipoma, neurofibroma, and fibroma), cancers of the large bowel or colon, (e.g., adenocarcinoma, tubular adenoma, villous adenoma, hamartoma, and leiomyoma), and other cancers of the digestive tract (e.g., anal cancer, anorectal cancer, appendix cancer, cancer of the anal canal, cancer of the tongue, gallbladder cancer, gastrointestinal stromal tumor (GIST), colon cancer, colorectal cancer, extrahepatic bile duct cancer, intrahepatic bile duct cancer, rectal cancer, and small intestine cancer); genitourinary tract cancers, including, for example, cancers of the kidney (e.g., adenocarcinoma, Wilm's tumor (nephroblastoma), lymphoma, and leukemia), cancers of the bladder and urethra (e.g., squamous cell carcinoma, transitional cell carcinoma, and adenocarcinoma), cancers of the prostate (e.g., adenocarcinoma and sarcoma), cancers of the testis, (e.g., seminoma, teratoma, embryonal carcinoma, teratocarcinoma, choriocarcinoma, sarcoma, interstitial cell carcinoma, fibroma, fibroadenoma, adenomatoid tumors, and lipoma), as well as transitional cell cancer, transitional cell cancer of the renal pelvis and ureter and other urinary organs, urethral cancer, and urinary bladder cancer; liver cancers, including, for example, hepatoma (e.g., hepatocellular carcinoma), cholangiocarcinoma, hepatoblastoma, angiosarcoma, hepatocellular adenoma, and hemangioma; bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochondroma (osteocartilaginous exostoses), benign chondroma, chondroblastoma, chondromyxofibroma, osteoid osteoma and giant cell tumors; nervous system cancers, including, for example, cancers of the skull (e.g., osteoma, hemangioma, granuloma, xanthoma, and osteitis deformans); cancers of the meninges (e.g., meningioma, meningiosarcoma, and gliomatosis); cancers of the brain (e.g., astrocytoma, medulloblastoma, glioma, ependymoma, germinoma (pinealoma), glioblastoma multiforme, oligodendroglioma, schwannoma, retinoblastoma, and congenital tumors); cancers of the spinal cord (e.g., neurofibroma, meningioma, glioma, and sarcoma), and other nervous system cancers (e.g., brain stem glioma, diffuse intrinsic pontine glioma (DIPG), brain tumor, central nervous system cancer, cerebellar astrocytoma, cerebral astrocytoma/malignant glioma, childhood cerebellar astrocytoma, childhood cerebral astrocytoma, primary central nervous system lymphoma, visual pathway and hypothalamic glioma, nervous system lymphoma, supratentorial primitive neuroectodermal tumors, pineoblastoma and supratentorial primitive neuroectodermal tumors); gynecological cancers, including, for example, cancers of the uterus (e.g., endometrial carcinoma), cancers of the cervix (e.g., cervical carcinoma, and pre tumor cervical dysplasia), cancers of the ovaries (e.g., ovarian carcinoma, including serous cystadenocarcinoma, mucinous cystadenocarcinoma, unclassified carcinoma, granulosa thecal cell tumors, Sertoli Leydig cell tumors, dysgerminoma, and malignant teratoma), cancers of the vulva (e.g., squamous cell carcinoma, intraepithelial carcinoma, adenocarcinoma, fibrosarcoma, and melanoma), cancers of the vagina (e.g., clear cell carcinoma, squamous cell carcinoma, botryoid sarcoma, and embryonal rhabdomyosarcoma), and cancers of the fallopian tubes (e.g., carcinoma); other reproductive tract cancers, including, for example, endometrial cancer, endometrial uterine cancer, germ cell tumor, gestational trophoblastic tumor, gestational trophoblastic tumor glioma, ovarian epithelial cancer, ovarian germ cell tumor, ovarian low malignant potential tumor, penile cancer, vaginal cancer, vulvar cancer, extracranial germ cell tumor, extragonadal germ cell tumor, uterine cancer, uterine corpus cancer, uterine sarcoma; lymphatic and hematologic cancers, including, for example, cancers of the blood (e.g., acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, and myelodysplastic syndrome, Hodgkin's lymphoma, non Hodgkin's lymphoma (malignant lymphoma) and Waldenstrom's macroglobulinemia), and other lymphatic or hematologic cancers including, for example, childhood leukemia, myeloproliferative disorders (e.g., primary myelofibrosis), plasma cell neoplasm/multiple myeloma, myelodysplasia, myelodysplastic syndrome, cutaneous T-cell lymphoma, lymphoid neoplasm, AIDS-related lymphoma, thymoma, thymoma and thymic carcinoma, mycosis fungoides, and Sézary Syndrome; skin cancers, including, for example, malignant melanoma, basal cell carcinoma, squamous cell carcinoma, Kaposi's sarcoma, moles dysplastic nevi, lipoma, angioma, dermatofibroma, keloids, psoriasis, merkel cell carcinoma, merkel cell skin carcinoma, melanoma, and carcinoid tumor; adrenal gland cancers, including, for example, neuroblastoma; other cancers associated with the endocrine system including, for example, adrenocortical carcinoma, multiple endocrine neoplasia (e.g., multiple endocrine neoplasia type I), multiple endocrine neoplasia syndrome, parathyroid cancer, pituitary tumor, pheochromocytoma, islet cell pancreatic cancer, and islet cell tumors); connective tissue cancer (e.g., bone cancer, bone and joint cancer, osteosarcoma and malignant fibrous histiocytoma); cancer associated with the head, neck, and mouth (e.g., head and neck cancer, paranasal sinus and nasal cavity cancer, metastatic squamous neck cancer, mouth cancer, throat cancer, esophageal cancer, laryngeal cancer, pharyngeal cancer, hypopharyngeal cancer, lip and oral cavity cancer, nasopharyngeal cancer, oral cancer, oropharyngeal cancer, and salivary gland cancer); and cancer associated with the eye (e.g., ocular cancer, intraocular melanoma). In some embodiments, the cancer is Ewing's sarcoma.

In some embodiments, the cancer is a hematological cancer such as leukemia or lymphoma. Example leukemia and lymphomas treatable by the compounds of the disclosure include mixed lineage leukemia (MLL), MLL-related leukemia, MLL-associated leukemia, MLL-positive leukemia, MLL-induced leukemia, rearranged mixed lineage leukemia (MLL-r), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL) (also referred to as acute lymphoblastic leukemia or acute lymphoid leukemia), acute myeloid leukemia (AML) (also referred to as acute myelogenous leukemia or acute myeloblastic leukemia), acute granulocytic leukemia, acute nonlymphocytic leukemia, chronic lymphocytic leukemia (CLL) (also referred to as chronic lymphoblastic leukemia), chronic myelogenous leukemia (CML) (also referred to as chronic myeloid leukemia), therapy related leukemia, myelodysplastic syndrome (MDS), myeloproliferative disease (MPD) (such as primary myelofibrosis (PMF)), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sezary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non-Hodgkin's lymphoma (malignant lymphoma), and Waldenstrom's macroglobulinemia. In some embodiments, the acute myeloid leukemia (AML) is abstract nucleophosmin (NPM1)-mutated acute myeloid leukemia (i.e., NPM1^{mut} acute myeloid leukemia).

In particular embodiments, compounds of the disclosure are used to treat a rearranged mixed lineage (KMT2A-rearranged) leukemia (MLL-r).

In particular embodiments, compounds of the disclosure are used to treat leukemia associated with a MLL rearrangement, acute lymphocytic leukemia associated with a MLL rearrangement, acute lymphoblastic leukemia associated with a MLL rearrangement, acute lymphoid leukemia associated with a MLL rearrangement, acute myeloid leukemia associated with a MLL rearrangement, acute myelogenous leukemia associated with a MLL rearrangement, or acute myeloblastic leukemia associated with a MLL rearrangement. As used herein, “MLL rearrangement” means a rearrangement of the MLL gene.

In some embodiments, diseases and conditions treatable with compounds of the disclosure include insulin resistance, pre-diabetes, diabetes (e.g., Type 2 diabetes or Type 1 diabetes), and risk of diabetes. In some embodiments, diseases and conditions treatable with compounds of the disclosure include hyperglycemia. In some embodiments, the hyperglycemia is associated with diabetes, such as Type 2 diabetes. In some embodiments, compounds of the disclosure are used to treat loss of response to other anti-diabetic agents and/or reduced beta cell function in a patient or subject. In some embodiments, compounds of the disclosure are used to restore response to other anti-diabetic agents and/or to restore beta cell function and/or to reduce the need for insulin in a patient or subject. In some embodiments, compounds of the disclosure are used to reduce insulin resistance, reduce the risk of diabetes, or reduce increases in blood glucose caused by a statin in a subject taking a statin. In some embodiments, compounds of the disclosure are used to treat diabetes in a subject taking a statin or to prevent diabetes in a subject taking a statin. Methods of the disclosure include decreasing, reducing, inhibiting, suppressing, limiting or controlling in the patient elevated blood glucose levels. In further aspects, methods of the disclosure include increasing, stimulating, enhancing, promoting, inducing or activating in the subject insulin sensitivity. Statins include, but are not limited to atorvastatin, cerivastatin, fluvastatin, lovastatin, mevastatin, pitavastatin, pravastatin, rosuvastatin and simvastatin.

In some embodiments, a patient is treated with (e.g., administered) a compound of the present disclosure in an amount sufficient to treat or ameliorate one or more of the diseases and conditions recited above (e.g., a therapeutically effective amount). The compounds of the disclosure may also be useful in the prevention of one or more of the diseases recited therein.

In some embodiments, a compound of the present disclosure or a pharmaceutically acceptable salt thereof, or a pharmaceutical composition of the present disclosure is used in treating or preventing a disease caused by, or associated with, menin expression, activity, and/or function.

In some embodiments, a compound of the present disclosure or a pharmaceutically acceptable salt thereof, or a pharmaceutical composition of the present disclosure is used in treating or preventing cancer.

In some embodiments, a compound of the present disclosure or a pharmaceutically acceptable salt thereof, or a pharmaceutical composition of the present disclosure is used for treating or preventing a disease caused by, or associated with, menin expression, activity, and/or function.

In some embodiments, a compound of the present disclosure or a pharmaceutically acceptable salt thereof, or a pharmaceutical composition of the present disclosure is used in the manufacture of a medicament for treating or preventing a disease caused by, or associated with, menin expression, activity, and/or function.

In some embodiments, the present disclosure relates to a kit comprising a compound of the present disclosure or a pharmaceutically acceptable salt thereof, or a pharmaceutical composition of the present disclosure and instructions for its use.

In some embodiments, a compound of the present disclosure or a pharmaceutically acceptable salt thereof, or a pharmaceutical composition of the present disclosure is used for treating hematological malignancies. In some embodiments, a compound of the present disclosure or a pharmaceutically acceptable salt thereof, or a pharmaceutical composition of the present disclosure is used for treating hematological malignancies as described in WO2023150635A1, which disclosure is incorporated by reference herein.

In some embodiments, a compound of the present disclosure or a pharmaceutically acceptable salt thereof, or a pharmaceutical composition of the present disclosure is used for treating hematologic cancer with high interferon regulatory factor 8 (IRF8) expression. In some embodiments, a compound of the present disclosure or a pharmaceutically acceptable salt thereof, or a pharmaceutical composition of the present disclosure is used for treating hematologic cancer with high interferon regulatory factor 8 (IRF8) expression as described in WO2025015152A1, which disclosure is incorporated by reference herein.

In some embodiments, a compound of the present disclosure or a pharmaceutically acceptable salt thereof, or a pharmaceutical composition of the present disclosure is used for treating PIK3CA mutation colon cancer. In some embodiments, a compound of the present disclosure or a pharmaceutically acceptable salt thereof, or a pharmaceutical composition of the present disclosure is used for treating PIK3CA mutation colon cancer as described in CN patent No. 118021815B, which disclosure is incorporated by reference herein.

In some embodiments, a compound of the present disclosure or a pharmaceutically acceptable salt thereof, or a pharmaceutical composition of the present disclosure is used for treating sickle cell disease. In some embodiments, a compound of the present disclosure or a pharmaceutically acceptable salt thereof, or a pharmaceutical composition of the present disclosure is used for treating sickle cell disease as described in U.S. Patent Publication No. 20240100049A1, which disclosure is incorporated by reference herein.

### Combination Therapy

The disclosure further relates to a combination therapy for treating a disease or a disorder described herein. In some embodiments, the combination therapy comprises administering at least one compound of the present disclosure in combination with one or more other pharmaceutically active agents for treating cancer or other disorders mediated by menin/MLL. In some embodiments, the combination therapy comprises administering at least one compound of the present disclosure in combination with one or more other pharmaceutically active agents, such as for the treatment of cancer. The pharmaceutically active agents can be combined with a compound of the disclosure in a single dosage form, or the therapeutics can be administered simultaneously or sequentially as separate dosage forms.

In some embodiments, the invention provides a combination therapy comprising a menin inhibitor of the present disclosure (e.g., a compound of Formula I′, Formula I, Formula I-a, Formula I-b, Formula I-c, etc.) and a CYP3A4 inhibitor. In certain embodiments, the invention provides for a pharmaceutical composition comprising: (a) a menin inhibitor of the present disclosure (e.g., a compound of Formula I′, Formula I, Formula I-a, Formula I-b, Formula I-c, etc.), and (b) a CYP3A4 inhibitor. In some embodiments, the invention is directed to a method for treating a patient comprising (a) administering a menin inhibitor of the present disclosure (e.g., a compound of Formula I′, Formula I, Formula I-a, Formula I-b, Formula I-c, etc.), and (b) administering a CYP3A4 inhibitor.

Some embodiments of this invention are directed to combination therapies designed to treat or manage cancer in a subject, wherein the combination therapies comprise administering a menin inhibitor of the present disclosure (e.g., a compound of Formula I′, Formula I, Formula I-a, Formula I-b, Formula I-c, etc.) in combination with a CYP3A4 inhibitor. In particular, some embodiments of this invention are directed to methods of treating or managing cancer in a subject, comprising administering a menin inhibitor in combination with a therapeutically effective amount of a CYP3A4 inhibitor administered simultaneously, separately or sequentially.

In some embodiments, the CYP3A inhibitor is: an antiarrhythmic; an antihistamine; an azole antifungal; a benzodiazepine; a calcium channel blocker; a HIV antiviral; a HMG CoA Reductase inhibitor; a macrolide antibiotic; a prokinetic; a protease inhibitor; or any combinations thereof. In some embodiments, the CYP3A inhibitor is: posaconazole, alprazolam; amiodarone; amlodipine; aprepitant; aripiprazole; astemizole; atorvastatin; boceprevir; buspirone; chloramphenicol; chlorpheniramine; cimetidine; ciprofloxacin; cisapride; clarithromycin; cobicistat (GS-9350); analogs or derivatives of cobicistat (GS-9350); cyclosporine; delaviridine; diazepam→3—OH; diethyl-dithiocarbamate; diltiazem; erythromycin; felodipine; fluconazole; fluvoxamine; gestodene; gleevec; grapefruit juice; haloperidol; imatinib; indinavir; itraconazole; ketoconazole; lovastatin; methadone; mibefradil; midazolam; mifepristone; nefazodone; nelfinavir; nifedipine; nisoldipine; nitrendipine; norfloxacin; norfluoxetine; pimozide; quinine; quinidine→3—OH; ritonavir; saquinavir; sildenafil; simvastatin; starfruit; tacrolimus (FK506); tamoxifen; telaprevir; telithromycin; trazodone; triazolam; verapamil; telaprevir; vincristine; voriconazole; or any combinations thereof.

In some embodiments, the CYP3A4 inhibitor is posaconazole, cobicistat (GS-9350) or analogs or derivatives of cobicistat (GS-9350). In some embodiments, the CYP3A4 inhibitor is ketoconazole. In some embodiments, the CYP3A4 inhibitor is ritonavir. In some embodiments, the menin inhibitor and the CYP3A4 inhibitor are in separate dosage forms. In some embodiments, the pharmaceutical composition is in a combined dosage form. In some embodiments, the CYP3A4 inhibitor is posaconazole.

In some embodiments, the pharmaceutical composition comprises an amount of the CYP3A4 inhibitor that is effective to increase the oral bioavailability of the menin inhibitor. The compounds according to the disclosure may also be used in combination with immunotherapies, including but not limited to cell-based therapies, antibody therapies and cytokine therapies, for the treatment of a disease or disorder disclosed herein.

In certain embodiments, compounds according to the disclosure are used in combination with one or more passive immunotherapies, including but not limited to naked monoclonal antibody drugs and conjugated monoclonal antibody drugs. Examples of naked monoclonal antibody drugs that can be used include, but are not limited to, rituximab (Rituxan®), an antibody against the CD20 antigen; trastuzumab (Herceptin®), an antibody against the HER2 protein; alemtuzumab (Lemtrada®, Campath®), an antibody against the CD52 antigen; cetuximab (Erbitux®), an antibody against the EGFR protein; and bevacizumab (Avastin®) which is an anti-angiogenesis inhibitor of VEGF protein.

Examples of conjugated monoclonal antibodies that can be used include, but are not limited to, radiolabeled antibody ibritumomab tiuxetan (Zevalin®); radiolabeled antibody tositumomab (Bexxar®); and immunotoxin gemtuzumab ozogamicin (Mylotarg®) which contains calicheamicin; BL22, an anti-CD22 monoclonal antibody-immunotoxin conjugate; radiolabeled antibodies such as OncoScint® and ProstaScint®; brentuximab vedotin (Adcetris®); ado-trastuzumab emtansine (Kadcyla®, also called TDM-1).

Further examples of therapeutic antibodies that can be used include, but are not limited to, REOPRO® (abciximab), an antibody against the glycoprotein IIb/IIIa receptor on platelets; ZENAPAX® (daclizumab) an immunosuppressive, humanized anti-CD25 monoclonal antibody; PANOREX™, a murine anti-17-IA cell surface antigen IgG2a antibody; BEC2, a murine anti-idiotype (GD3 epitope) IgG antibody; IMC-C225, a chimeric anti-EGFR IgG antibody; VITAXIN™ a humanized anti-αVβ3 integrin antibody; Campath 1H/LDP-03, a humanized anti CD52 IgG1 antibody; Smart M195, a humanized anti-CD33 IgG antibody; LYMPHOCIDE™, a humanized anti-CD22 IgG antibody; LYMPHOCIDE™ Y-90; Lymphoscan; Nuvion® (against CD3; CM3, a humanized anti-ICAM3 antibody; IDEC-114 a primatized anti-CD80 antibody; IDEC-131 a humanized anti-CD40L antibody; IDEC-151 a primatized anti-CD4 antibody; IDEC-152 a primatized anti-CD23 antibody; SMART anti-CD3, a humanized anti-CD3 IgG; 5G1.1, a humanized anti-complement factor 5 (C5) antibody; D2E7, a humanized anti-TNF-α antibody; CDP870, a humanized anti-TNF-α Fab fragment; IDEC-151, a primatized anti-CD4 IgG1 antibody; MDX-CD4, a human anti-CD4 IgG antibody; CD20-streptavidin (+biotin-yttrium 90); CDP571, a humanized anti-TNF-α IgG4 antibody; LDP-02, a humanized anti-α4β7 antibody; OrthoClone OKT4A, a humanized anti-CD4 IgG antibody; ANTOVA™, a humanized anti-CD40L IgG antibody; ANTEGREN™, a humanized anti-VLA-4 IgG antibody; and CAT-152, a human anti-TGF-β₂ antibody.

In certain embodiments, compounds according to the disclosure are used in combination with one or more targeted immunotherapies containing toxins but not an antibody, including but not limited to denileukin diftitox (Ontak®), IL-2 linked to diphtheria toxin.

The compounds according to the disclosure may also be used in combination with adjuvant immunotherapies for the treatment of a disease or disorder disclosed herein. Such adjuvant immunotherapies include, but are not limited to, cytokines, such as granulocyte-macrophage colony-stimulating factor (GM-CSF), granulocyte-colony stimulating factor (G-CSF), macrophage inflammatory protein (MIP)-1-alpha, interleukins (including IL-1, IL-2, IL-4, IL-6, IL-7, IL-12, IL-15, IL-18, IL-21, and IL-27), tumor necrosis factors (including TNF-alpha), and interferons (including IFN-alpha, IFN-beta, and IFN-gamma); aluminum hydroxide (alum); Bacille Calmette-Guerin (BCG); Keyhole limpet hemocyanin (KLH); Incomplete Freund's adjuvant (IFA); QS-21; DETOX; Levamisole; and Dinitrophenyl (DNP), and combinations thereof, such as, for example, combinations of interleukins, for example IL-2, with other cytokines, such as IFN-alpha.

In certain embodiments, compounds according to the disclosure are used in combination with vaccine therapy, including but not limited to autologous and allogeneic tumor cell vaccines, antigen vaccines (including polyvalent antigen vaccines), dendritic cell vaccines, and viral vaccines.

In another embodiment, the present disclosure comprises administering to a subject with cancer an effective amount of a compound of the disclosure and one or more additional anti-cancer therapies selected from: surgery, anti-cancer agents/drugs, biological therapy, radiation therapy, anti-angiogenesis therapy, immunotherapy, adoptive transfer of effector cells, gene therapy or hormonal therapy. Examples of anti-cancer agents/drugs are described below.

In some embodiments, the anti-cancer agents/drug is, for example, adriamycin, actinomycin, bleomycin, vinblastine, cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; palbociclib; Yervoy® (ipilimumab); Mekinist™ (trametinib); peginterferon alfa-2b, recombinant interferon alfa-2b; Sylatron™ (peginterferon alfa-2b); Tafinlar® (dabrafenib); Zelboraf® (vemurafenib); or nivolumab.

The compounds according to the present disclosure can be administered in combination with existing methods of treating cancers, for example by chemotherapy, irradiation, or surgery. Thus, there is further provided a method of treating cancer comprising administering an effective amount of a compound of the disclosure, or a pharmaceutically acceptable salt form thereof, to a subject in need of such treatment, wherein an effective amount of at least one additional cancer chemotherapeutic agent is administered to the subject. Examples of suitable cancer chemotherapeutic agents include any of: abarelix, ado-trastuzumab emtansine, aldesleukin, alemtuzumab, alitretinoin, allopurinol, altretamine, anastrozole, arsenic trioxide, asparaginase, azacitidine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, emtansine, epirubicin, eribulin, erlotinib, estramustine, etoposide phosphate, etoposide, everolimus, exemestane, fentanyl citrate, filgrastim, floxuridine, fludarabine, fluorouracil, fruquintinib, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, ixabepilone, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, paclitaxel albumin-stabilized nanoparticle formulation, pamidronate, panitumumab, pegaspargase, pegfilgrastim, pemetrexed disodium, pentostatin, pertuzuma, pipobroman, plicamycin, procarbazine, quinacrine, rasburicase, rituximab, sorafenib, streptozocin, sulfatinib, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, volitinib, vorinostat, and zoledronate.

In particular embodiments, compounds according to the disclosure are used in combination with one or more anti-cancer agent selected from methotrexate, paclitaxel albumin-stabilized nanoparticle formulation, ado-trastuzumab emtansine, eribulin, doxorubicin, fluorouracil, everolimus, anastrozole, pamidronate disodium, exemestane, capecitabine, cyclophosphamide, docetaxel, epirubicin, toremifene, fulvestrant, letrozole, gemcitabine, gemcitabine hydrochloride, goserelin acetate, trastuzumab, ixabepilone, lapatinib ditosylate, megestrol acetate, tamoxifen citrate, pamidronate disodium, palbociclib, and pertuzumab for the treatment of breast cancer.

Other anti-cancer agents/drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; andrographolide; angiogenesis inhibitors; antagonist D; antagonist G; antarelix; anti-dorsalizing morphogenetic protein-1; antiandrogen; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors; castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclin-dependent kinase inhibitors; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; elfomithine; elemene; emitefur; epirubicin; epristeride; estramustine analogue; estrogen agonists; estrogen antagonists; etanidazole; etoposide phosphate; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitors; microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentine; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; zanoterone; zilascorb; zinostatin stimalamer; 5-fluorouracil; and leucovorin.

In some embodiments, the anti-cancer agent/drug is an agent that stabilizes microtubules. As used herein, a “microtubulin stabilizer” means an anti-cancer agent/drug which acts by arresting cells in the G2-M phases due to stabilization of microtubules. Examples of microtubulin stabilizers include ACLITAXEL® and Taxol® analogues. Additional examples of microtubulin stabilizers include without limitation the following marketed drugs and drugs in development: Discodermolide (also known as NVP-XX-A-296); Epothilones (such as Epothilone A, Epothilone B, Epothilone C (also known as desoxyepothilone A or dEpoA); Epothilone D (also referred to as KOS-862, dEpoB, and desoxyepothilone B); Epothilone E; Epothilone F; Epothilone B N-oxide; Epothilone A N-oxide; 16-aza-epothilone B; 21-aminoepothilone B (also known as BMS-310705); 21-hydroxyepothilone D (also known as Desoxyepothilone F and dEpoF), 26-fluoroepothilone); FR-182877 (Fujisawa, also known as WS-9885B), BSF-223651 (BASF, also known as ILX-651 and LU-223651); AC-7739 (Ajinomoto, also known as AVE-8063A and CS-39·HCl); AC-7700 (Ajinomoto, also known as AVE-8062, AVE-8062A, CS-39-L-Ser.HCl, and RPR-258062A); Fijianolide B; Laulimalide; Caribaeoside; Caribaeolin; Taccalonolide; Eleutherobin; Sarcodictyin; Laulimalide; Dictyostatin-1; Jatrophane esters; and analogs and derivatives thereof.

In another embodiment, the anti-cancer agent/drug is an agent that inhibits microtubules. As used herein, a “microtubulin inhibitor” means an anti-cancer agent which acts by inhibiting tubulin polymerization or microtubule assembly. Examples of microtubulin inhibitors include without limitation the following marketed drugs and drugs in development: Erbulozole (also known as R-55104); Dolastatin 10 (also known as DLS-10 and NSC-376128); Mivobulin isethionate (also known as CI-980); Vincristine; NSC-639829; ABT-751 (Abbott, also known as E-7010); Altorhyrtins (such as Altorhyrtin A and Altorhyrtin C); Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9); Cemadotin hydrochloride (also known as LU-103793 and NSC-D-669356); Auristatin PE (also known as NSC-654663); Soblidotin (also known as TZT-1027), LS-4559-P (Pharmacia, also known as LS-4577); LS-4578 (Pharmacia, also known as LS-477-P); LS-4477 (Pharmacia), LS-4559 (Pharmacia); RPR-112378 (Aventis); Vincristine sulfate; DZ-3358 (Daiichi); GS-164 (Takeda); GS-198 (Takeda); KAR-2 (Hungarian Academy of Sciences); SAH-49960 (Lilly/Novartis); SDZ-268970 (Lilly/Novartis); AM-97 (Armad/Kyowa Hakko); AM-132 (Armad); AM-138 (Armad/Kyowa Hakko); IDN-5005 (Indena); Cryptophycin 52 (also known as LY-355703); Vitilevuamide; Tubulysin A; Canadensol; Centaureidin (also known as NSC-106969); T-138067 (Tularik, also known as T-67, TL-138067 and TI-138067); COBRA-1 (Parker Hughes Institute, also known as DDE-261 and WHI-261); H10 (Kansas State University); H16 (Kansas State University); Oncocidin A1 (also known as BTO-956 and DIME); DDE-313 (Parker Hughes Institute); SPA-2 (Parker Hughes Institute); SPA-1 (Parker Hughes Institute, also known as SPIKET-P); 3-IAABU (Cytoskeleton/Mt. Sinai School of Medicine, also known as MF-569); Narcosine (also known as NSC-5366); Nascapine, D-24851 (Asta Medica), A-105972 (Abbott); Hemiasterlin; 3-BAABU (Cytoskeleton/Mt. Sinai School of Medicine, also known as MF-191); TMPN (Arizona State University); Vanadocene acetylacetonate; T-138026 (Tularik); Monsatrol; Indanocine (also known as NSC-698666); 3-IAABE (Cytoskeleton/Mt. Sinai School of Medicine); A-204197 (Abbott); T-607 (Tularik, also known as T-900607); RPR-115781 (Aventis); Eleutherobins (such as Desmethyleleutherobin, Desaetyleleutherobin, Isoeleutherobin A, and Z-Eleutherobin); Halichondrin B; D-64131 (Asta Medica); D-68144 (Asta Medica); Diazonamide A; A-293620 (Abbott); NPI-2350 (Nereus); TUB-245 (Aventis); A-259754 (Abbott); Diozostatin; (−)-Phenylahistin (also known as NSCL-96F037); D-68838 (Asta Medica); D-68836 (Asta Medica); Myoseverin B; D-43411 (Zentaris, also known as D-81862); A-289099 (Abbott); A-318315 (Abbott); HTI-286 (also known as SPA-110, trifluoroacetate salt) (Wyeth); D-82317 (Zentaris); D-82318 (Zentaris); SC-12983 (NCI); Resverastatin phosphate sodium; BPR-OY-007 (National Health Research Institutes); SSR-250411 (Sanofi); Combretastatin A4; eribulin (Halaven®); and analogs and derivatives thereof.

In further embodiments, compounds according to the disclosure are used in combination with one or more alkylating agents, antimetabolites, natural products, or hormones.

Examples of alkylating agents useful in the methods of the disclosure include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.).

Examples of antimetabolites useful in the methods of the disclosure include but are not limited to folic acid analog (e.g., methotrexate), or pyrimidine analogs (e.g., fluorouracil, floxouridine, cytarabine), and purine analogs (e.g., mercaptopurine, thioguanine, pentostatin). Examples of natural products useful in the methods of the disclosure include but are not limited to vinca alkaloids (e.g., vinblastin, vincristine), epipodophyllotoxins (e.g., etoposide, teniposide), antibiotics (e.g., actinomycin D, daunorubicin, doxorubicin, bleomycin, plicamycin, mitomycin) or enzymes (e.g., L-asparaginase).

Examples of hormones and antagonists useful for the treatment of cancer include but are not limited to adrenocorticosteroids (e.g., prednisone), progestins (e.g., hydroxyprogesterone caproate, megestrol acetate, medroxyprogesterone acetate), estrogens (e.g., diethylstilbestrol, ethinyl estradiol), antiestrogen (e.g., tamoxifen), androgens (e.g., testosterone propionate, fluoxymesterone), antiandrogen (e.g., flutamide), and gonadotropin releasing hormone analog (e.g., leuprolide).

Other agents that can be used in combination with the compounds of the disclosure for the treatment of cancer include platinum coordination complexes (e.g., cisplatin, carboplatin), anthracenedione (e.g., mitoxantrone), substituted urea (e.g., hydroxyurea), methyl hydrazine derivative (e.g., procarbazine), and adrenocortical suppressant (e.g., mitotane, aminoglutethimide). Other anti-cancer agents/drugs that can be used in combination with the compounds of the disclosure include, but are not limited to, liver X receptor (LXR) modulators, including LXR agonists and LXR beta-selective agonists; aryl hydrocarbon receptor (AhR) inhibitors; inhibitors of the enzyme poly ADP ribose polymerase (PARP), including olaparib, iniparib, rucaparib, veliparib; inhibitors of vascular endothelial growth factor (VEGF) receptor tyrosine kinases, including cediranib; programmed cell death protein 1 (PD-1) inhibitors, including nivolumab (Bristol-Myers Squibb Co.) and pembrolizumab (Merck & Co., Inc.; MK-3475); MEK inhibitors, including cobimetinib; B-Raf enzyme inhibitors, including vemurafenib; cytotoxic T lymphocyte antigen (CTLA-4) inhibitors, including tremelimumab; programmed death-ligand 1 (PD-L1) inhibitors, including MEDI4736 (AstraZeneca); inhibitors of the Wnt pathway; inhibitors of epidermal growth factor receptor (EGFR) including AZD9291 (AstraZeneca), erlotinib, gefitinib, panitumumab, and cetuximab; adenosine A2A receptor inhibitors; adenosine A2B receptor inhibitors; colony-stimulating factor-1 receptor (CSF1R) inhibitors, including PLX3397 (Plexxikon), and inhibitors of CD73.

The compounds of the disclosure can be used in combination with one or more therapeutic strategies including immune checkpoint inhibitors, including inhibitors of PD-1, PD-L1, and CTLA-4.

The compounds of the disclosure can be used in combination with one or more anti-cancer agents selected from MCL-1 inhibitors, e.g., homoharringtonine (HHT) and omacetaxine; BCL-2 inhibitors, e.g., venetoclax (ABT-199), navitoclax (ABT-263), ABT-737, gossypol (AT-101), apogossypolone (ApoG2) and obatoclax; selective inhibitors of nuclear export (SINEs), e.g., selinexor (KPT-330).

In particular embodiments, the compounds of the disclosure are used in combination with one or more anti-cancer agents selected from methotrexate (Abitrexate®; Folex®; Folex PFS®; Mexate®; Mexate-AQ®); nelarabine (Arranon®); blinatumomab (Blincyto®); rubidomycin hydrochloride or daunorubicin hydrochloride (Cerubidine®); cyclophosphamide (Clafen®; Cytoxan®; Neosar®); clofarabine (Clofarex®; Clolar®); cytarabine (Cytosar-U®; Tarabine PFS®); dasatinib (Sprycel®); doxorubicin hydrochloride; asparaginase Erwinia chrysanthemi (Erwinaze); imatinib mesylate (Gleevec®); ponatinib hydrochloride (Iclusig®); mercaptopurine (Purinethol; Purixan); pegaspargase (Oncaspar®); prednisone; vincristine sulfate (Oncovin®, Vincasar PFS®, Vincrex®); vincristine sulfate liposome (Marqibo®); hyper-CVAD (fractionated cyclophosphamide, vincristine, adriamycin, and dexamethasone); arsenic trioxide (Trisenox®); idarubicin hydrochloride (Idamycin®); mitoxantrone hydrochloride; thioguanine (Tabloid®); ADE (cytarabine, daunorubicin, and etoposide); alemtuzumab (Lemtrada®, Campath®); chlorambucil (Ambochlorin®, Amboclorin®, Leukeran®, Linfolizin®); ofatumumab (Arzerra); bendamustine hydrochloride (Treanda®); fludarabine phosphate (Fludara®); obinutuzumab (Gazyva®); ibrutinib (Imbruvica®); idelalisib (Zydelig®); mechlorethamine hydrochloride (Mustargen®); rituximab (Rituxan®); chlorambucil-prednisone; CVP (cyclophosphamide, vincristine, and prednisone); bosutinib (Bosulif®); busulfan (Busulfex®; Myleran®); omacetaxine mepesuccinate (Synribo®); nilotinib (Tasigna®); Intron® A (recombinant interferon Alfa-2b); DOT1L inhibitors, including EPZ-5676 (Epizyme, Inc.); and inhibitors of bromodomain and extra-terminal motif (BET) proteins (BET inhibitors), including MS417, JQ1, I-BET 762, and I-BET 151 for the treatment of leukemia.

Compounds of the disclosure can be used in combination with one or more other agents or therapies for the treatment of insulin resistance, pre-diabetes, diabetes (e.g., Type 2 diabetes or Type 1 diabetes), and risk of diabetes, including but not limited to insulins and insulin analogues, such as Humulin® (Eli Lilly), Lantus® (Sanofi Aventis), Novolin® (Novo Nordisk), and Exubera® (Pfizer); Avandamet® (metformin HCI and rosiglitazone maleate, GSK); Avandaryl® (glimepiride and rosiglitazone maleate, GSK); Metaglip® (glipizide and metformin HCI, Bristol Myers Squibb); Glucovance® (glyburide and metformin HCI, Bristol Myers Squibb); PPAR gamma agonists, such as Avandia® (rosiglitizone maleate, GSK) and Actos® (pioglitazone hydrochloride, Takeda/Eli Lilly); sulfonylureas, such as Amaryl® (glimepiride, Sanofi Aventis), Diabeta® (glyburide, Sanofi Aventis), Micronase®/Glynase® (glyburide, Pfizer), and Glucotrol®/Glucotrol XL® (glipizide, Pfizer); meglitinides, such as Prandin®/NovoNorm® (repaglinide, Novo Nordisk), Starlix® (nateglinide, Novartis), and Glufast® (mitiglinide, Takeda); biguanides, such as Glucophase®/Glucophase XR® (metformin HCI, Bristol Myers Squibb) and Glumetza® (metformin HCI, Depomed); thiazolidinediones; amylin analogs; GLP-1 analogs; DPP-IV inhibitors such as Januvia® (sitagliptin, Merck) and Galvus' (vildagliptin, Novartis); PTB-1 B inhibitors; protein kinase inhibitors (including AMP-activated protein kinase inhibitors); glucagon antagonists, glycogen synthase kinase-3 beta inhibitors; glucose-6-phosphatase inhibitors; glycogen phosphorylase inhibitors; sodium glucose co-transporter inhibitors; and alpha-glucosidase inhibitors, such as Glycet® (miglitol, Pfizer); statins, fibrates, and Zetia® (ezetimibe); alpha-blockers; beta-blockers; calcium channel blockers; diuretics; angiotensin converting enzyme (ACE) inhibitors; dual ACE and neutral endopeptidase (NEP) inhibitors; angiotensin-receptor blockers (ARBs); aldosterone synthase inhibitors; aldosterone-receptor antagonists; endothelin receptor antagonists; orlistat; phentermine; sibutramine; Acomplia® (rimonabant); thiazolidinediones (e.g., rosiglitazone, pioglitazone); SGLT 2 inhibitors (e.g., dapagliflozin, remogliflozin etabonate, sergliflozin, canagliflozin, and 1-chloro-4-(β-D-glucopyranos-1-yl)-2-[4-(('S)-tetrahydrofuran-3-yloxy)-benzyl]-benzene); PPAR-gamma-agonists (e.g., Gl 262570) and antagonists; PPAR-gamma/alpha modulators (e.g., KRP 297); alpha-glucosidase inhibitors (e.g., acarbose, voglibose); DPPIV inhibitors (e.g., Januvia® (sitagliptin), Galvus®/Zomelis® (vildagliptin), Onglyza® (saxagliptin), Nesina®/Nipidia® (alogliptin), and Tradjenta®/Trajenta® (linagliptin)); alpha2-antagonists; glucagon-like protein-1 (GLP-1) receptor agonists and analogues (e.g., exendin-4); amylin; inhibitors of protein tyrosinephosphatase 1; substances that affect deregulated glucose production in the liver, e.g., inhibitors of glucose-6-phosphatase, or fructose-1,6-bisphosphatase, glycogen phosphorylase; glucagon receptor antagonists; inhibitors of phosphoenol pyruvate carboxykinase; glycogen synthase kinase and glucokinase activators; lipid lowering agents such as HMG-CoA-reductase inhibitors (e.g., simvastatin, atorvastatin); fibrates (e.g., bezafibrate, fenofibrate), nicotinic acid and the derivatives thereof, PPAR-alpha agonists, PPAR-delta agonists; ACAT inhibitors (e.g., avasimibe); cholesterol absorption inhibitors such as ezetimibe; bile acid-binding substances such as cholestyramine; inhibitors of ileac bile acid transport; HDL-raising compounds such as CETP inhibitors and ABC1 regulators; active substances for treating obesity such as sibutramine and tetrahydrobiopterin; SDRIs; axokine; leptin; leptin mimetics; antagonists of the cannabinoid 1 receptor; and MCH-1 receptor antagonists; MC4 receptor agonists; NPY5 and NPY2 antagonists; beta3 adrenergic agonists such as SB-418790 and AD-9677; agonists of the 5HT2c receptor; GABA-receptor antagonists; Na-channel blockers; topiramate; protein-kinase C inhibitors; advanced glycation end product inhibitors; and aldose reductase inhibitors.

### Pharmaceutical Formulations, Administration, and Dosage Forms

When employed as pharmaceuticals, the compounds of the disclosure can be administered in the form of a pharmaceutical composition which refers to a combination of a compound of the disclosure, or its pharmaceutically acceptable salt, and at least one pharmaceutically acceptable carrier. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal, or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, one or more of the compounds of the disclosure above in combination with one or more pharmaceutically acceptable carriers. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

Compounds or compositions described herein may be administered to a patient using any amount and any route of administration effective for treating or lessening the severity of one or more of the diseases and conditions described herein. The exact amount required will vary from subject to subject, depending on the species, age, and general condition of the subject, the severity of the infection, disease or disorder, the particular agent, its mode of administration, and the like. Provided compounds are preferably formulated in a particular unit dosage form for ease of administration and uniformity of dosage. The expression “unit dosage form” as used herein refers to a physically discrete unit of agent appropriate for the patient to be treated.

The therapeutic dosage of the compounds of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. The dosage is likely to depend on such variables as the type and extent of progression of the disease or disorder, the overall health status of the particular patient, the relative biological efficacy of the compound selected, formulation of the excipient, and its route of administration.

### NUMBERED EMBODIMENTS

1. A compound of Formula I′,

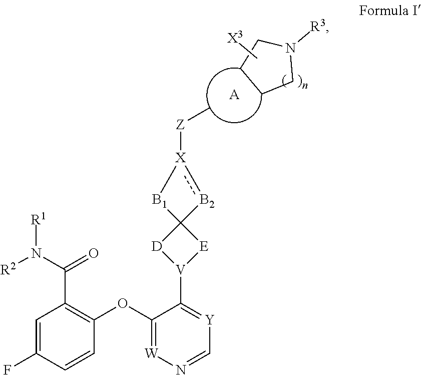

a stereoisomer, or a pharmaceutically acceptable salt thereof, wherein:
[symbol] denotes a single bond or a double bond, as valency permits;
B₁, B₂, D, and E are each independently selected from —C(R^{A1})(R^{A2}), —C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})C(═O)—, and —N═C(NH₂)— wherein no more than one of B₁, B₂, D, and E is —C(R^{A1})(R^{A2})—C(R^{A1})(R^{A2})—NR^{A3}—, —C(R^{A1})(R^{A2})—C(═O)—, —C(═O)—, or —N═C(NH₂)—;
V is N or CR^{W}, wherein R^{W} is H, halo, CN, OH, C₁-C₄ alkyl, C₁-C₄ alkoxy, amino, C₁-C₄ alkyl amino, or C₂-C₈ dialkylamino;
X is N or CR^{X}, wherein R^{X} is absent, H, halo, CN, OH, C₁-C₄ alkyl, C₁-C₄ alkoxy, amino, C₁-C₄ alkyl amino, or C₂-C₈ dialkylamino;
each R^{A1} is independently selected from absent, H, halo, C₁-C₄ alkyl, C₁-C₄ alkoxy, C₁-C₄ haloalkyl, C₁-C₄ haloalkoxy, amino, C₁-C₄ alkylamino, C₂-C₈ dialkylamino, CN, NO₂, and OH;
each R^{A2} is independently selected from H, halo, C₁-C₄ alkyl, C₁-C₄ alkoxy, C₁-C₄ haloalkyl, C₁-C₄ haloalkoxy, amino, C₁-C₄ alkylamino, C₂-C₈ dialkylamino, CN, NO₂, and OH;
each R^{A3} is independently selected from H, C₁-C₄ alkyl, C₁-C₄ alkoxy, C₁-C₄ haloalkyl, C(O)R^{z}, and C(O)OR^{z}, wherein said C₁-C₄ alkyl is optionally substituted by phenyl, C₁-C₄ alkoxy, C₁-C₄ haloalkoxy, CN, NO₂, or OH;
R is H, C₁-C₄ alkyl, or phenyl;
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Z is O, CH₂, or NH;
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a′})(R^{4b′}), or N(R^{4a′})(R^{4b′});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.
2. The compound of embodiment 1, a stereoisomer, or a pharmaceutically acceptable salt thereof, wherein when [symbol] is a double bond, X is C.
3. The compound of any one of the preceding embodiments, a stereoisomer, or a pharmaceutically acceptable salt thereof, wherein the spiro moiety represented by the below formula:

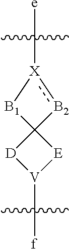

wherein e and f indicate points of attachment to the remainder of the molecule, is selected from:

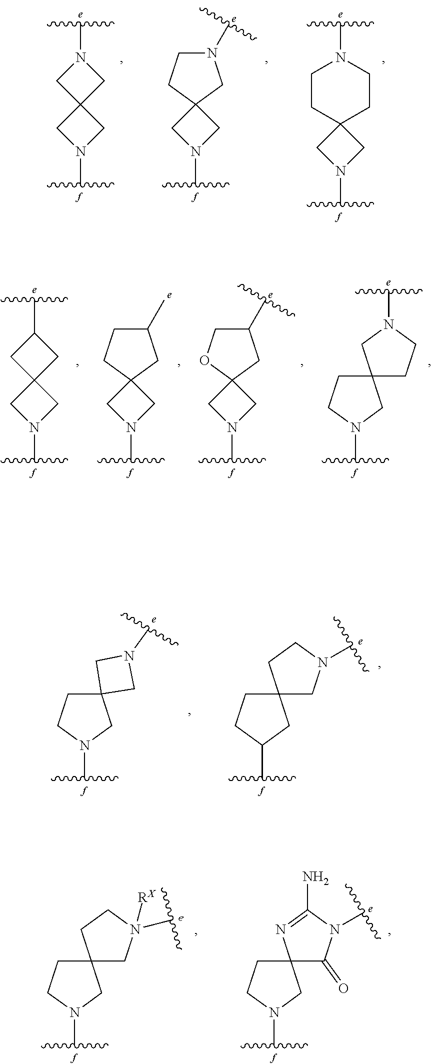

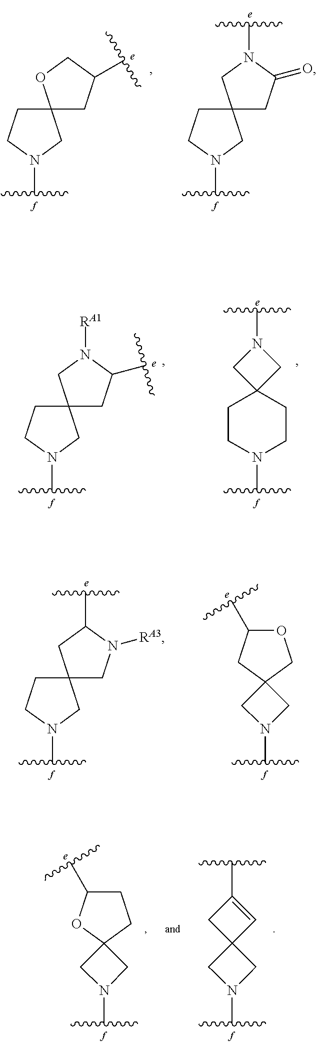

4. A compound of Formula I,

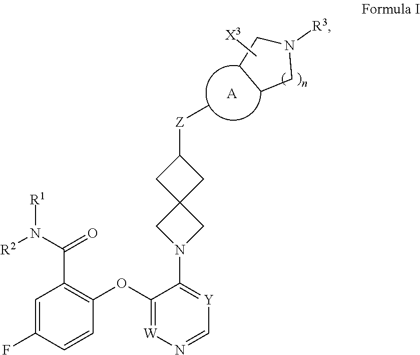

a stereoisomer, or a pharmaceutically acceptable salt thereof, wherein:
W is N or CH,
Y is N or CH,
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{1bs};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Z is O, CH₂, or NH,
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.

5. A compound of Formula I-a,

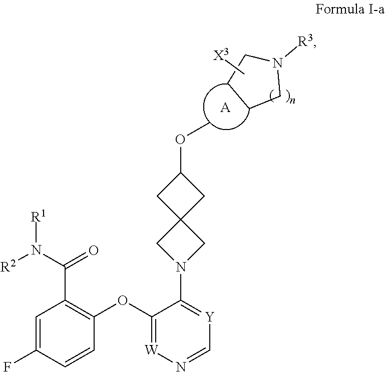

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.
6. A compound of Formula I-b,

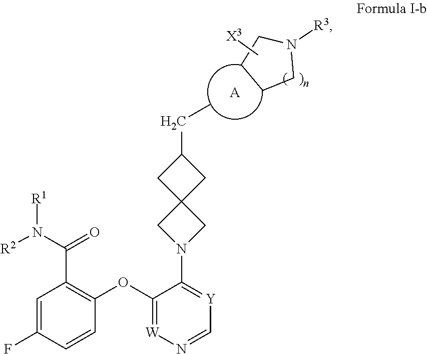

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.
7. A compound of Formula I-c,

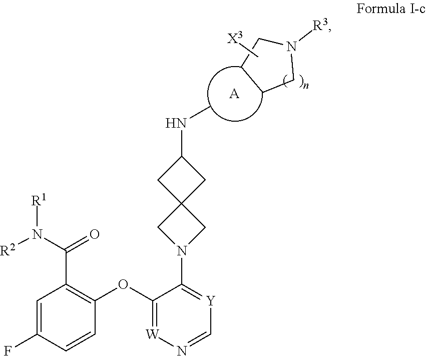

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′},
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR⁴, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl; each R^{4a′} and R^{4b′} is independently H, C₁-C₆ alkyl, C₃-C₁₂ cycloalkyl, or 3- to 12-membered heterocyclyl; and
n is 1, 2, or 3.
8. The compound of any one of the preceding embodiments, wherein W is N.
9. The compound of any one of the preceding embodiments, wherein W is CH.
10. The compound of any one of the preceding embodiments, wherein Y is N.
11. The compound of any one of the preceding embodiments, wherein Y is CH.
12. The compound of any one of the preceding embodiments, wherein R² is C₁-C₆ alkyl optionally substituted with one or more halo, OH, oxo, CN, or N(R^{4a′})(R^{4b′}).
13. The compound of any one of the preceding embodiments, wherein R² is isopropyl.
14. The compound of any one of the preceding embodiments, wherein Z is O.
15. The compound of any one of the preceding embodiments, wherein Z is CH₂.
16. The compound of any one of the preceding embodiments, wherein Z is NH.
17. The compound of any one of embodiments 1-16, wherein R¹ is —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a}.
18. The compound of any one of embodiments 1-17, wherein R¹ is —C₁-C₆ alkylene-N(R^{3a′})(R^{3b′}) wherein the C₁-C₆ alkylene is optionally substituted with one or more R^{1as};
   each R^{1as} is independently halo, oxo, C₁-C₆ alkyl, C₁-C₆ haloalkyl, C(═O)(R^{3a′}),
   N(R^{3a′})(R^{3b′}), or S(═O)₂R^{3a}.
19. The compound of embodiment 18, wherein R¹ is

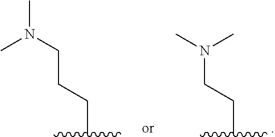

20. The compound of any one of embodiments 1-17, wherein R¹ is —C₃-C₁₂ cycloalkylene-N(R^{3a′})(R^{3b′}), wherein the C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b}′), or S(═O)₂R^{3a}.
21. The compound of embodiment 20, wherein R¹ is

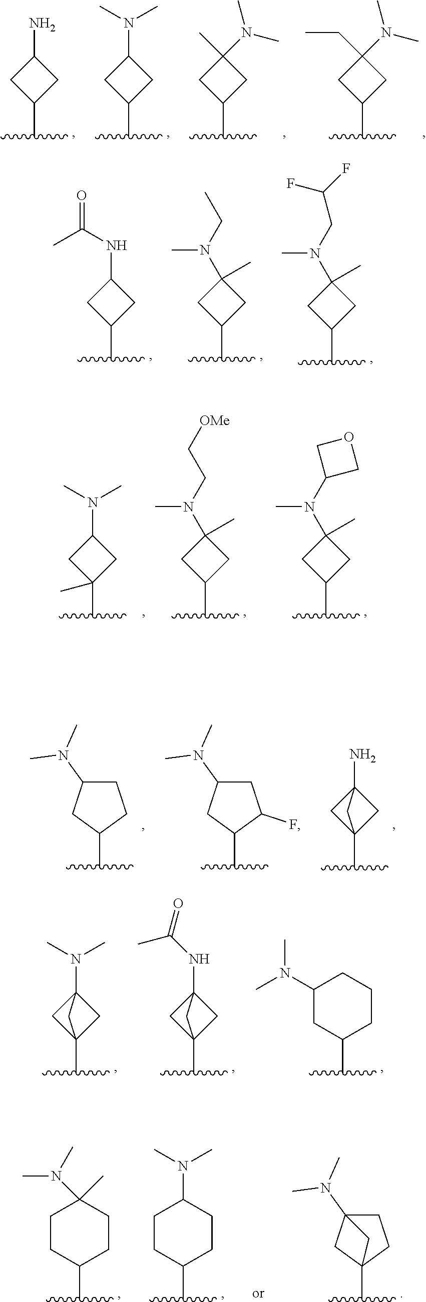

22. The compound of any one of embodiments 1-16, wherein R¹ is 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}.
23. The compound of embodiment 22, wherein R¹ is

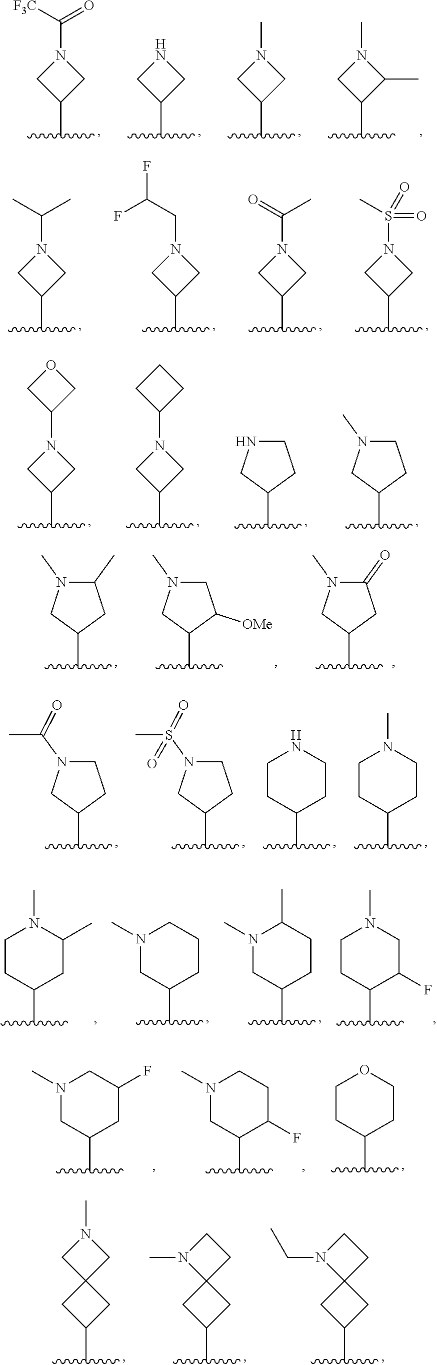

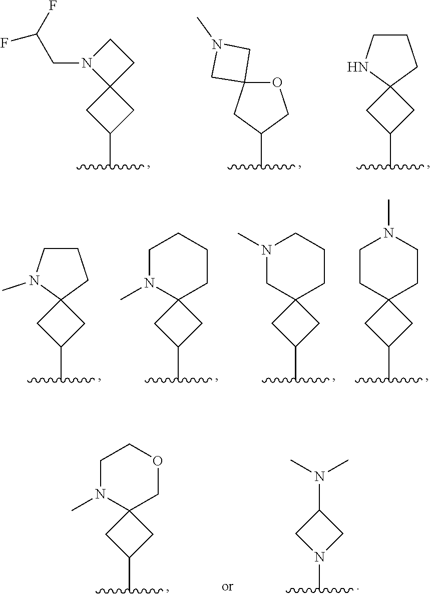

24. The compound of any one of embodiments 1-16, wherein R¹ is C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy.
25. The compound of embodiment 24, wherein R¹ is

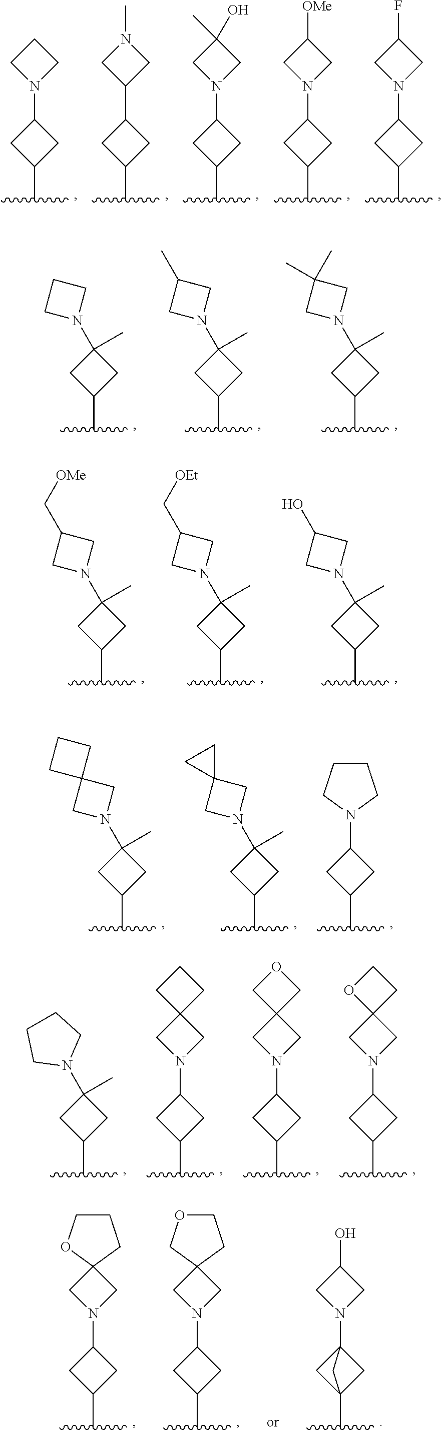

26. The compound of any one of the preceding embodiments, wherein Ring A is 6- to 10-membered aryl optionally substituted with one or more C₁-C₆ alkyl, halo, OH, CN, or C₁-C₆ alkoxy.
27. The compound of any one of the preceding embodiments, wherein Ring A is 6- to 10-membered aryl.
28. The compound of any one of embodiments 1-25, wherein Ring A is 6- to 10-membered heteroaryl optionally substituted with one or more C₁-C₆ alkyl, halo, OH, CN, or C₁-C₆ alkoxy.
29. The compound of any one of embodiments 1-25 and 28, wherein Ring A is 6- to 10-membered heteroaryl.
30. The compound of any one of embodiments 1-27, wherein Ring A is

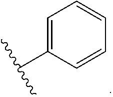 [SMILES: *c1ccccc1]

31. The compound of any one of embodiments 1-25, 28, and 29, wherein Ring A is

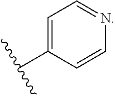 [SMILES: *c1ccncc1]

32. The compound of any one of embodiments 1-25, wherein

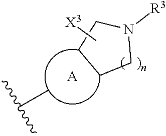

is

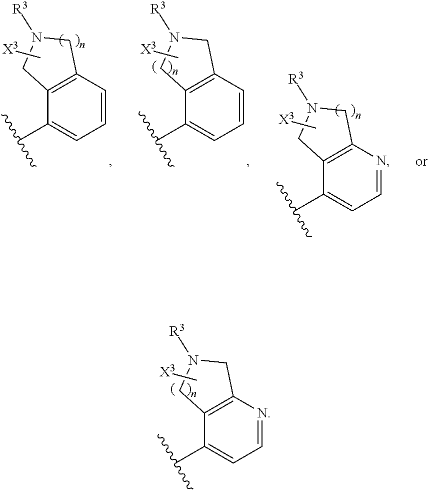

33. The compound of embodiment 32, wherein

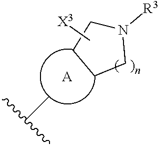

is

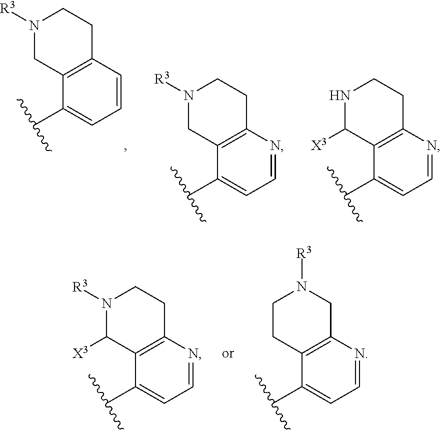

34. The compound of embodiment 32, wherein

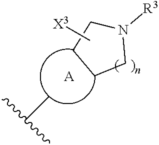

is

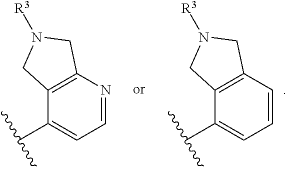

35. The compound of embodiment 32, wherein

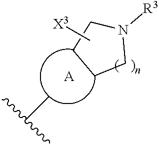

is

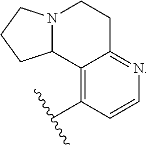 [SMILES: *c1ccnc2c1C1CCCN1CC2]

36. The compound of any one of embodiments 1-33, wherein X³ is C₁-C₆ alkyl optionally substituted with one or more halo, OR^{4a}, oxo, CN, C(═O)N(R^{4a})(R^{4b}), or N(R^{4a})(R^{4b}).
37. The compound of any one of embodiments 1-33 and 36, wherein X³ is C₁-C₆ alkyl.
38. The compound of any one of embodiments 1-33, 36, and 37, wherein X³ is methyl or ethyl.
39. The compound of any one of embodiments 1-33, wherein X³ is H.
40. The compound of any one of the preceding embodiments, wherein R³ is C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a};
   each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a}), and 3- to 12-membered heterocyclyl that is optionally substituted with oxo;
   each R^{4b} is independently H, CN, or C₁-C₆ alkyl optionally substituted with C₆-C₁₀ aryl or N(R^{4a′})(R^{4b′});
   each R^{4a} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′}); and
   each R^{4a′} and R^{4b′} is independently H, C₁-C₆ alkyl, C₃-C₁₂ cycloalkyl, or 3- to 12-membered heterocyclyl.
41. The compound of any one of the preceding embodiments, wherein R³ is C₁-C₆ alkyl optionally substituted with OR^{4a}, and R^{4a} is H, CN, or C₁-C₆ alkyl.
42. The compound of any one of the preceding embodiments, wherein R³ is methyl, ethyl, isopropyl or tert-butyl.
43. The compound of any one of embodiments 1-41, wherein R³ is

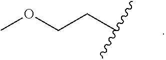 [SMILES: *CCOC]

44. The compound of any one of embodiments 1-40, wherein R³ is C₁-C₆ alkyl optionally substituted with C₆-C₁₀ aryl or 3- to 12-membered heterocyclyl.
45. The compound of any one of embodiments 1-40 and 44, wherein R³ is or

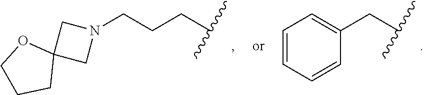 [SMILES: *CCCN1CC2(CCCO2)C1 ; *Cc1ccccc1]

46. The compound of any one of embodiments 1-40, wherein R³ is C(O)(R^{4a}) or C(O)O—C(R^{4a})₂—OC(O)(R^{4a}), wherein:
   each R^{4a} is independently H, CN, or C₁-C₆ alkyl optionally substituted with C₆-C₁₀ aryl or N(R^{4a′})(R^{4b′}); and
   each R^{4a′} and R^{4b′} is independently H, C₁-C₆ alkyl, C₃-C₁₂ cycloalkyl, or 3- to 12-membered heterocyclyl.
47. The compound of any one of embodiments 1-40 and 46, wherein R³ is

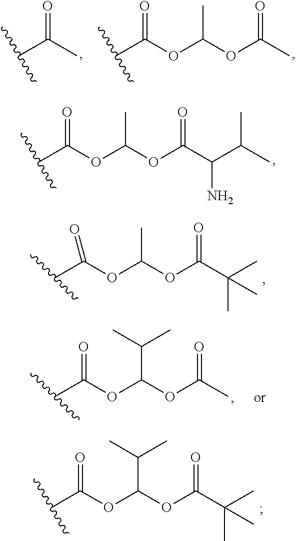

48. The compound ofany one of embodiments 1-39, wherein R³ is H.
49. The compound of any one of embodiments 1-25 and 35, wherein R³ forms a 3- to 12-membered heterocyclyl with the carbon atom next to the nitrogen atom to which it is connected.
50. The compound of any one of the preceding embodiments, wherein n is 1.
51. The compound of any one of embodiments 1-49, wherein n is 2.
52. The compound of any one of embodiments 1-49, wherein n is 3.
53. A compound as shown in Table 1 or a stereoisomer thereof, or a pharmaceutically acceptable salt thereof.
54. A compound as shown in Table 1 or a pharmaceutically acceptable salt thereof.
55. A compound as shown in Table 1.
56. A compound as shown in Table 1 or a pharmaceutically acceptable salt thereof, wherein the salt is hydrochloride.
57. The compound according to any one of the preceding embodiments, wherein the compound is useful for the treatment of cancer and wherein the compound minimizes hERG binding.
58. A pharmaceutical composition comprising a compound of any one of the preceding embodiments, or a pharmaceutically acceptable salt thereof, and at least one pharmaceutically acceptable carrier.
59. A pharmaceutical composition comprising a salt or crystalline form of the compound of any one of embodiments 1-57, and at least one pharmaceutically acceptable carrier.
60. A method of inhibiting the interaction between menin and MLL comprising contacting the menin and MLL with a compound of any one of embodiments 1-57 or a pharmaceutical composition of embodiment 58 or embodiment 59.
61. A method of treating cancer in a patient comprising administering to the patient a compound of any one of embodiments 1-57 or a pharmaceutical composition of embodiment 58 or embodiment 59.
62. The method of embodiment 61, wherein the cancer is a hematological cancer.
63. The method of embodiment 61, wherein the cancer is a leukemia.
64. The method of embodiment 61, wherein the cancer is a lymphoma.
65. The method of embodiment 61, wherein the cancer is mixed lineage leukemia (MLL), MLL-related leukemia, MLL-associated leukemia, MLL-positive leukemia, MLL-induced leukemia, rearranged mixed lineage (KMT2A-rearranged)leukemia (MLL-r), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL), acute myeloid leukemia (AML), acute granulocytic leukemia, acute nonlymphocytic leukemia, chronic lymphocytic leukemia (CLL), chronic myelogenous leukemia (CML), therapy related leukemia, myelodysplastic syndrome (MDS), myeloproliferative disease (MPD), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sezary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non Hodgkin's lymphoma (malignant lymphoma), or Waldenstrom's macroglobulinemia.
66. The method of embodiment 61, wherein the cancer is an abstract nucleophosmin (NPM1)-mutated acute myeloid leukemia (i.e., NPM1^{mut} acute myeloid leukemia).
67. The method of embodiment 61, wherein the cancer is a rearranged mixed lineage (KMT2A-rearranged) leukemia (MLL-r).
68. The compound of any one of embodiments 1-57 or a pharmaceutically acceptable salt thereof, or the pharmaceutical composition of embodiment 58 or embodiment 59, for use in treating or preventing a disease caused by, or associated with, menin expression, activity, and/or function.
69. The compound of any one of embodiments 1-57 or a pharmaceutically acceptable salt thereof, or the pharmaceutical composition of embodiment 58 or embodiment 59, for use in treating or preventing cancer.
70. Use of the compound of any one of embodiments 1-57 or a pharmaceutically acceptable salt thereof, or the pharmaceutical composition of embodiment 58 or embodiment 59, for treating or preventing a disease caused by, or associated with, menin expression, activity, and/or function.
71. Use of the compound of any one of embodiments 1-57 or a pharmaceutically acceptable salt thereof, or the pharmaceutical composition of embodiment 58 or embodiment 59, in the manufacture of a medicament for treating or preventing a disease caused by, or associated with, menin expression, activity, and/or function.
72. Use of a compound of any of embodiments 1-57 or a pharmaceutically acceptable salt thereof, or the pharmaceutical composition of embodiment 58 or embodiment 59, for treating or preventing cancer.
73. Use of the compound of any one of embodiments 1-57 or a pharmaceutically acceptable salt thereof, or the pharmaceutical composition of embodiment 58 or embodiment 59, in the manufacture of a medicament for treating or preventing cancer.
74. A kit comprising the compound of any one of embodiments 1-57 or a pharmaceutically acceptable salt thereof, or the pharmaceutical composition of embodiment 58 or embodiment 59 and instructions for its use.
75. A method of preparing a compound of any one of embodiments 1-57 or a pharmaceutically acceptable salt thereof, according to a scheme of the present disclosure or synthetic description in the Examples.
76. An intermediate useful in the preparation of any one of embodiments 1-57 or a pharmaceutically acceptable salt thereof.

### Numbered Embodiments B

B1. A compound of Formula I′,

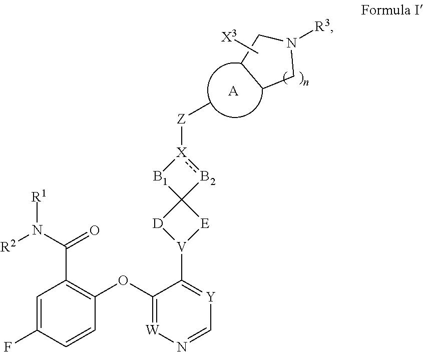

a stereoisomer, or a pharmaceutically acceptable salt thereof, wherein:
[symbol] denotes a single bond or a double bond, as valency permits;
B₁, B₂, D, and E are each independently selected from —C(R^{A1})(R^{A2})—, —C(R^{A1})(R^{A2})—C(R^{A1})(R^{A2})—, —C(R^{A1})(R^{A2})—O, —C(R^{A1})(R^{A2})—NR^{A3}—, —C(═O)—, —C(R^{A1})(R^{A2})—C(═O)—, and —N═C(NH₂)— wherein no more than one of B1, B2, D, and E is —C(R^{A1})(R^{A2})—, —(R^{A1})(R^{A2})—NR³—, —C(R^{A1})(R^{A2})—C(═O)—, —C(═O)—, or —N═C(NH₂)—;
V is N or CR^{X}, wherein R^{X} is H, halo, CN, OH, C₁-C₄ alkyl, C₁-C₄ alkoxy, amino, C₁-C₄ alkyl amino, or C₂-C₈ dialkylamino;
X is N or CR^{X}, wherein R^{X} is absent, H, halo, CN, OH, C₁-C₄ alkyl, C₁-C₄ alkoxy, amino, C₁-C₄ alkyl amino, or C₂-C₈ dialkylamino;
each R^{A1} is independently selected from absent, H, halo, C₁-C₄ alkyl, C₁-C₄ alkoxy, C₁-C₄ haloalkyl, C₁-C₄ haloalkoxy, amino, C₁-C₄ alkylamino, C₂-C₈ dialkylamino, CN, NO₂, and OH;
each R^{A2} is independently selected from H, halo, C₁-C₄ alkyl, C₁-C₄ alkoxy, C₁-C₄ haloalkyl, C₁-C₄ haloalkoxy, amino, C₁-C₄ alkylamino, C₂-C₈ dialkylamino, CN, NO₂, and OH;
each R^{A3} is independently selected from H, C₁-C₄ alkyl, C₁-C₄ alkoxy, C₁-C₄ haloalkyl, C(O)R^{z}, and C(O)OR^{z}, wherein said C₁-C₄ alkyl is optionally substituted by phenyl, C₁-C₄ alkoxy, C₁-C₄ haloalkoxy, CN, NO₂, or OH;
R is H, C₁-C₄ alkyl, or phenyl;
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Z is O, CH₂, or NH;
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(Ra), and 3- to 12-membered heterocyclyl that is optionally substituted with oxo;
each R¹ is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.
B2. The compound of embodiment 1, a stereoisomer, or a pharmaceutically acceptable salt thereof, wherein when [symbol] is a double bond, X is C.
B3. The compound of any one of the preceding embodiments, a stereoisomer, or a pharmaceutically acceptable salt thereof, wherein the spiro moiety represented by the below formula:

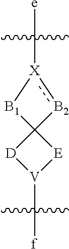

wherein e and f indicate points of attachment to the remainder of the molecule, is selected from:

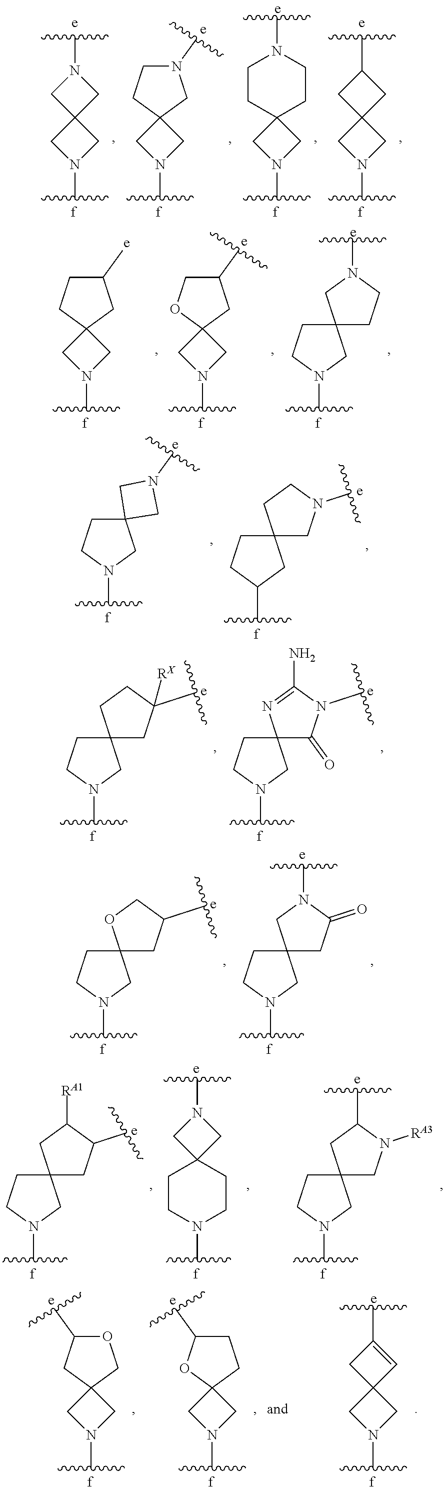

B4. A compound of Formula I,

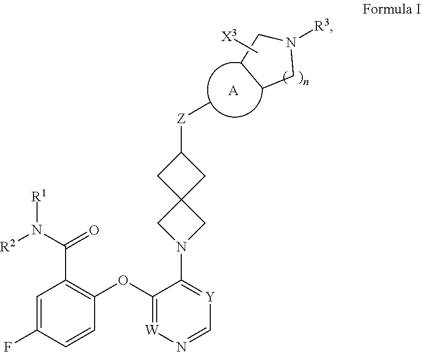

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
(c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4′})R^{4′});
Z is O, CH₂, or NH;
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4b′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.
B5. A compound of Formula I-a,

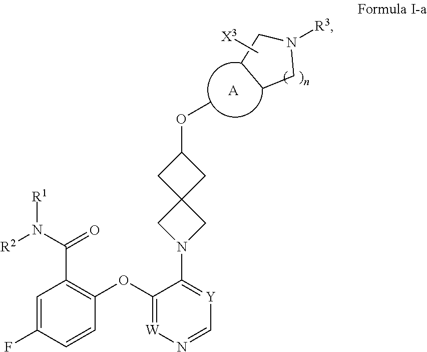

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R′), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(Ra), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.
B6. A compound of Formula I-b,

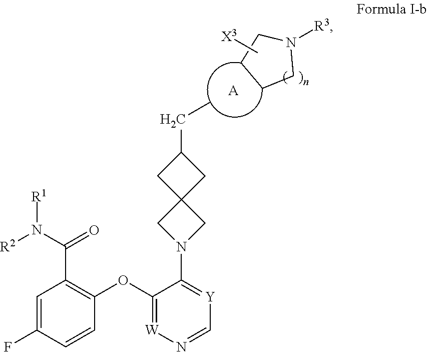

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a}), oxo, CN, C(O)OR^{4a′}, OC(O)N(R^{4a′})(R^{4b′}), N(R^{4a′})(R^{4b′}), N(R^{4a})C(O)(R^{4b′}), N(R^{4a})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.
B7. A compound of Formula I-c,

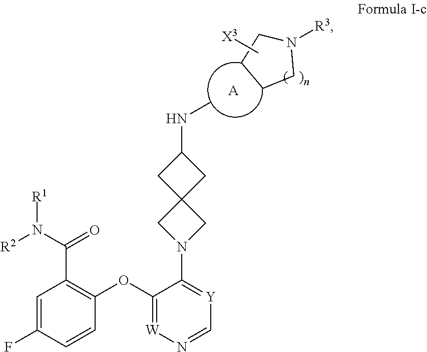

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR⁴, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a′}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.
B8. The compound of any one of the preceding embodiments, wherein W is N.
B9. The compound of any one of the preceding embodiments, wherein W is CH.
B10. The compound of any one of the preceding embodiments, wherein Y is N.
B11. The compound of any one of the preceding embodiments, wherein Y is CH.
B12. The compound of any one of the preceding embodiments, wherein R² is C₁-C₆ alkyl optionally substituted with one or more halo, OH, oxo, CN, or N(R^{4a′})(R^{4b′}).
B13. The compound of any one of the preceding embodiments, wherein R² is isopropyl.
B14. The compound of any one of the preceding embodiments, wherein Z is O.
B15. The compound of any one of the preceding embodiments, wherein Z is CH₂.
B16. The compound of any one of the preceding embodiments, wherein Z is NH.
B17. The compound of any one of embodiments B1-B16, wherein R¹ is —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a}.
B18. The compound of any one of embodiments B1-B17, wherein R¹ is —C₁-C₆ alkylene-N(R^{3a′})(R^{3b′}) wherein the C₁-C₆ alkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a}.
B19. The compound of embodiment B18, wherein R¹ is

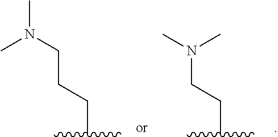

B20. The compound of any one of embodiments B1-B17, wherein R¹ is —C₃-C₁₂ cycloalkylene-N(R^{3a′})(R^{3b′}), wherein the C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a}.
B21. The compound of embodiment B20, wherein R¹ is

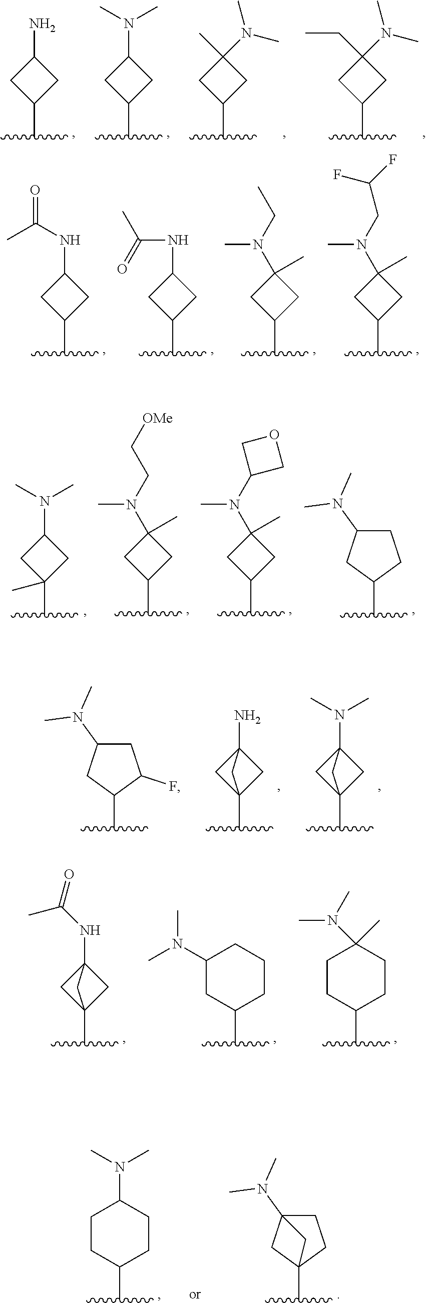

B22. The compound of any one of embodiments B1-B16, wherein R¹ is 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}.
B23. The compound of embodiment B22, wherein R¹ is

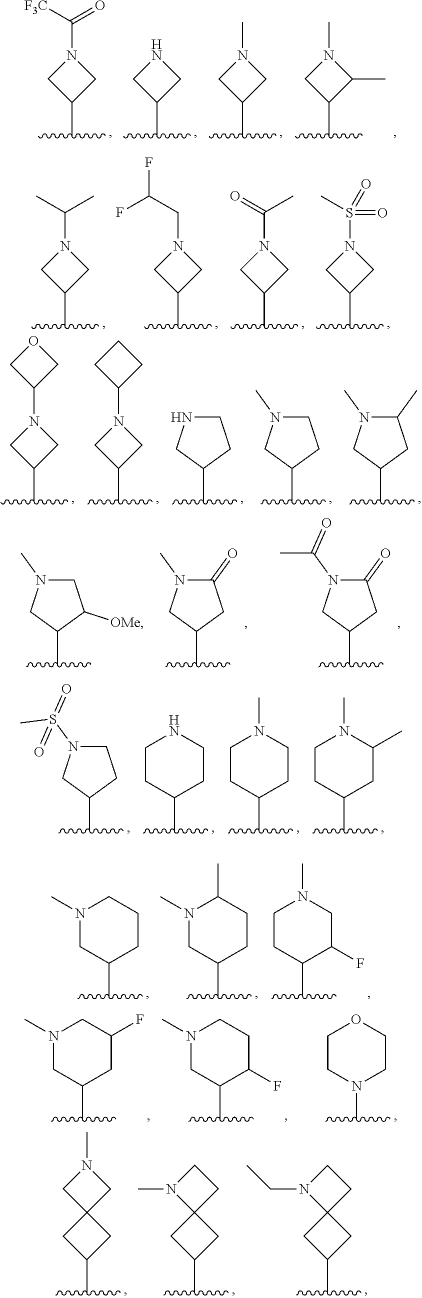

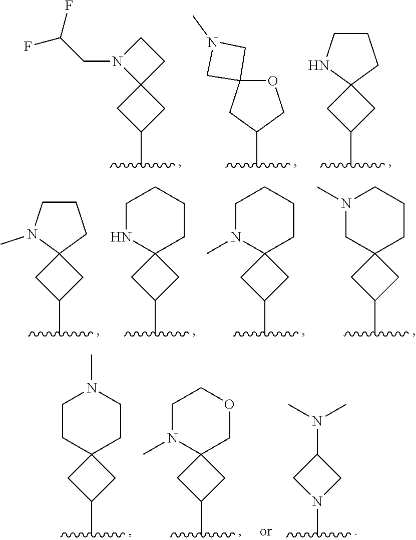

B24. The compound of any one of embodiments B1-B16, wherein R¹ is C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy.
B25. The compound of embodiment 24, wherein R¹ is

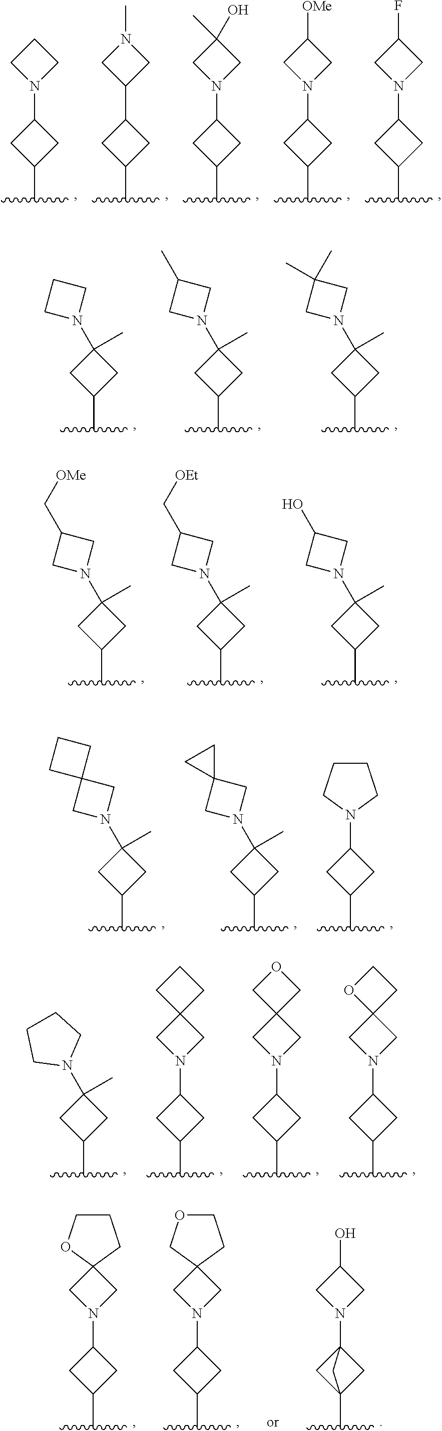

B26. The compound of any one of the preceding embodiments, wherein Ring A is 6- to 10-membered aryl optionally substituted with one or more C₁-C₆ alkyl, halo, OH, CN, or C₁-C₆ alkoxy.
B27. The compound of any one of the preceding embodiments, wherein Ring A is 6- to 10-membered aryl.
B28. The compound of any one of embodiments B1-B25, wherein Ring A is 6- to 10-membered heteroaryl optionally substituted with one or more C₁-C₆ alkyl, halo, OH, CN, or C₁-C₆ alkoxy.
B29. The compound of any one of embodiments B1-B25 and B28, wherein Ring A is 6- to 10-membered heteroaryl.
B30. The compound of any one of embodiments B1-B27, wherein Ring A is

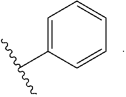 [SMILES: *c1ccccc1]

B31. The compound of any one of embodiments B1-B25, B28, and B29, wherein Ring A is

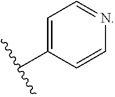 [SMILES: *c1ccncc1]

B32. The compound of any one of embodiments B1-B25, wherein

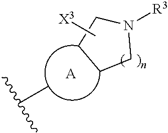

is

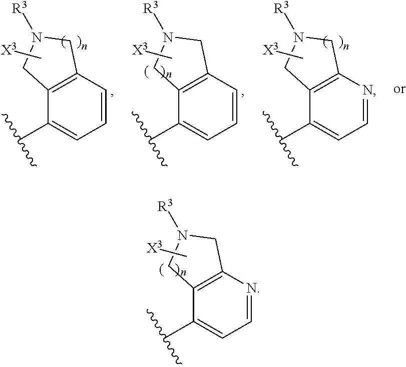

B33. The compound of embodiment B32, wherein

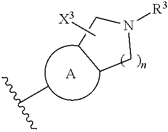

is

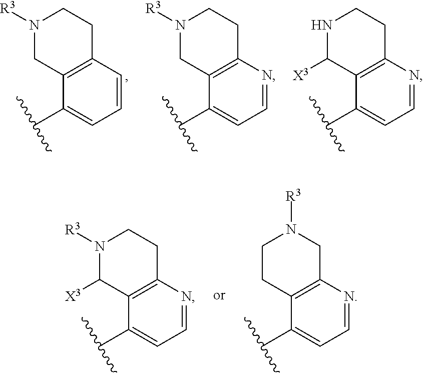

B34. The compound of embodiment B32, wherein

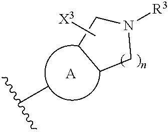

is

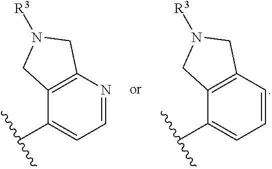

B35. The compound of embodiment B32, wherein

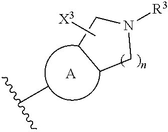

is

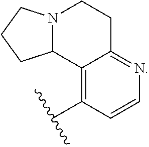 [SMILES: *c1ccnc2c1C1CCCN1CC2]

B36. The compound of any one of embodiments B1-B33, wherein X³ is C₁-C₆ alkyl optionally substituted with one or more halo, OR^{4a}, oxo, CN, C(═O)N(R^{4a})(R^{4b}), or N(R^{4a})(R^{4b}).
B37. The compound of any one of embodiments B1-B33 and B36, wherein X³ is C₁-C₆ alkyl.
B38. The compound of any one of embodiments B1-B33, B36, and B37, wherein X³ is methyl or ethyl.
B39. The compound of any one of embodiments B1-B33, wherein X³ is H.
B40. The compound of any one of the preceding embodiments, wherein R³ is C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a};
   each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a′}), and 3- to 12-membered heterocyclyl that is optionally substituted with oxo;
   each R^{4a} is independently H, CN, or C₁-C₆ alkyl optionally substituted with C₆-C₁₀ aryl or N(R^{4a′})(R^{4b′});
   each R^{4a} is independently H, S(═O)₂R^{4b′}, C(O)OR^{4b′}, C(O)R^{4b′}, C(O)NR^{4a′}R^{4b′}, C(O)CH₂—N(R^{4a′})R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′}); and
   each R^{4a′} and R^{4b′} is independently H, C₁-C₆ alkyl, C₃-C₁₂ cycloalkyl, or 3- to 12-membered heterocyclyl.
B41. The compound of any one of the preceding embodiments, wherein R³ is C₁-C₆ alkyl optionally substituted with OR^{4a}, and R^{4a} is H, CN, or C₁-C₆ alkyl.
B42. The compound of any one of the preceding embodiments, wherein R³ is methyl, ethyl, isopropyl or tert-butyl.
B43. The compound of any one of embodiments B1-B41, wherein R³ is

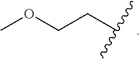 [SMILES: *CCOC]

B44. The compound of any one of embodiments B1-B40, wherein R³ is C₁-C₆ alkyl optionally substituted with C₆-C₁₀ aryl or 3- to 12-membered heterocyclyl.
B45. The compound of any one of embodiments B1-B40 and B44, wherein R³ is

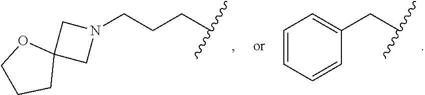 [SMILES: *CCCN1CC2(CCCO2)C1 ; *Cc1ccccc1]

B46. The compound of any one of embodiments B1-B40, wherein R³ is C(O)(R₄ₐ) or C(O)O—C(R^{4a})₂—OC(O)(R^{4a}), wherein:
   each R^{4a} is independently H, CN, or C₁-C₆ alkyl optionally substituted with C₆-C₁₀ aryl or N(R^{4a′})(R^{4b′}); and
   each R^{4a′} and R^{4b′} is independently H, C₁-C₆ alkyl, C₃-C₁₂ cycloalkyl, or 3- to 12-membered heterocyclyl.
B47. The compound of any one of embodiments B1-B40 and B46, wherein R³ is

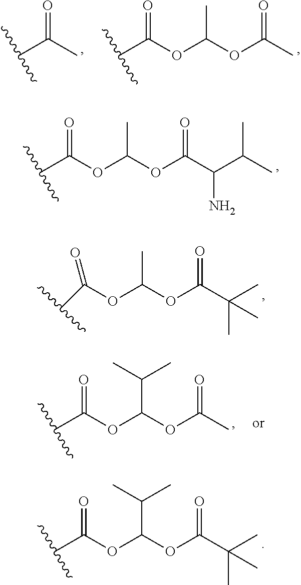

B48. The compound of any one of embodiments B1-B39, wherein R³ is H.
B49. The compound of any one of embodiments B1-B25 and B35, wherein R³ forms a 3- to 12-membered heterocyclyl with the carbon atom next to the nitrogen atom to which it is connected.
B50. The compound of any one of the preceding embodiments, wherein n is 1.
B51. The compound of any one of embodiments B1-B49, wherein n is 2.
B52. The compound of any one of embodiments B1-B49, wherein n is 3.
B53. A compound as shown in Table 1 or a stereoisomer thereof, or a pharmaceutically acceptable salt thereof.
B54. A compound as shown in Table 1 or a pharmaceutically acceptable salt thereof.
B55. A compound as shown in Table 1.
B56. A compound as shown in Table 1 or a pharmaceutically acceptable salt thereof, wherein the salt is hydrochloride.
B57. The compound according to any one of the preceding embodiments, wherein the compound is useful for the treatment of cancer and wherein the compound minimizes hERG binding.
B58. A pharmaceutical composition comprising a compound of any one of the preceding embodiments, or a pharmaceutically acceptable salt thereof, and at least one pharmaceutically acceptable carrier.
B59. A pharmaceutical composition comprising a salt or crystalline form of the compound of any one of embodiments B1-B57, and at least one pharmaceutically acceptable carrier.
B60. A method of inhibiting the interaction between menin and MLL comprising contacting the menin and MLL with a compound of any one of embodiments B1-B57 or a pharmaceutical composition of embodiment B58 or embodiment B59.
B61. A method of treating cancer in a patient comprising administering to the patient a compound of any one of embodiments 1-57 or a pharmaceutical composition of embodiment 58 or embodiment B59.
B62. The method of embodiment B61, wherein the cancer is a hematological cancer.
B63. The method of embodiment B61, wherein the cancer is a leukemia.
B64. The method of embodiment B61, wherein the cancer is a lymphoma.
B65. The method of embodiment B61, wherein the cancer is mixed lineage leukemia (MLL), MLL-related leukemia, MLL-associated leukemia, MLL-positive leukemia, MLL-induced leukemia, rearranged mixed lineage (KMT2A-rearranged)leukemia (MLL-r), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL), acute myeloid leukemia (AML), acute granulocytic leukemia, acute nonlymphocytic leukemia, chronic lymphocytic leukemia (CLL), chronic myelogenous leukemia (CML), therapy related leukemia, myelodysplastic syndrome (MDS), myeloproliferative disease (MPD), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sezary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non Hodgkin's lymphoma (malignant lymphoma), or Waldenstrom's macroglobulinemia.
B66. The method of embodiment B61, wherein the cancer is an abstract nucleophosmin (NPM1)-mutated acute myeloid leukemia (i.e., NPM1^{mut} acute myeloid leukemia).
B67. The method of embodiment B61, wherein the cancer is a rearranged mixed lineage (KMT2A-rearranged) leukemia (MLL-r).
B68. The compound of any one of embodiments B1-B57 or a pharmaceutically acceptable salt thereof, or the pharmaceutical composition of embodiment B58 or embodiment B59, for use in treating or preventing a disease caused by, or associated with, menin expression, activity, and/or function.
B69. The compound of any one of embodiments B1-B57 or a pharmaceutically acceptable salt thereof, or the pharmaceutical composition of embodiment B58 or embodiment B59, for use in treating or preventing cancer.
B70. Use of the compound of any one of embodiments B1-B57 or a pharmaceutically acceptable salt thereof, or the pharmaceutical composition of embodiment B58 or embodiment B59, for treating or preventing a disease caused by, or associated with, menin expression, activity, and/or function.
B71. Use of the compound of any one of embodiments B1-B57 or a pharmaceutically acceptable salt thereof, or the pharmaceutical composition of embodiment B58 or embodiment B59, in the manufacture of a medicament for treating or preventing a disease caused by, or associated with, menin expression, activity, and/or function.
B72. Use of a compound of any of embodiments B1-B57 or a pharmaceutically acceptable salt thereof, or the pharmaceutical composition of embodiment B58 or embodiment B59, for treating or preventing cancer.
B73. Use of the compound of any one of embodiments B1-B57 or a pharmaceutically acceptable salt thereof, or the pharmaceutical composition of embodiment B58 or embodiment B59, in the manufacture of a medicament for treating or preventing cancer.
B74. A kit comprising the compound of any one of embodiments B1-B57 or a pharmaceutically acceptable salt thereof, or the pharmaceutical composition of embodiment B58 or embodiment
B59 and instructions for its use.
B75. A method of preparing a compound of any one of embodiments B1-B57 or a pharmaceutically acceptable salt thereof, according to a scheme of the present disclosure or synthetic description in the Examples.
B76. An intermediate useful in the preparation of any one of embodiments B1-B57 or a pharmaceutically acceptable salt thereof.

### Numbered Embodiments C

C1. A compound of Formula I′,

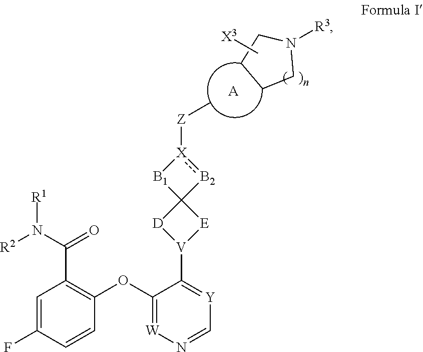

a stereoisomer a pharmaceutically acceptable salt thereof, wherein:
[symbol] denotes a single bond or a double bond, as valency permits;
B₁, B₂, D, and E are each independently selected from —C(R^{A1})(R^{A2})—, —C(R^{A1})(R^{A2})—(R^{A1})(R^{A2})—, —C(R^{A1})(R^{A2})—O, —C(R^{A1})(R^{A2})—NR^{A3}—, —C(═O)—, —C(R^{A1})(R^{A2})—C(═O)—, and —N═C(NH₂)— wherein no more than one of B1, B2, D, and E is —C(R^{A1})(R^{A2})—, —(R^{A1})(R^{A2})—NR³—, —C(R^{A1})(R^{A2})—C(═O)—, —C(═O)—, or —N═C(NH₂)—;
V is N or CR^{W}, wherein R^{W} is H, halo, CN, OH, C₁-C₄ alkyl, C₁-C₄ alkoxy, amino, C₁-C₄ alkyl amino, or C₂-C₈ dialkylamino;
X is N or CR^{X}, wherein R^{X} is absent, H, halo, CN, OH, C₁-C₄ alkyl, C₁-C₄ alkoxy, amino, C₁-C₄ alkyl amino, or C₂-C₈ dialkylamino;
each R^{A1} is independently selected from absent. H, halo, C₁-C₄ alkyl, C₁-C₄ alkoxy, C₁-C₄ haloalkyl, C₁-C₄ haloalkoxy, amino, C₁-C₄ alkylamino, C₂-C₈ dialkylamino, CN, NO₂, and OH;
each R^{A2} is independently selected from H, halo, C₁-C₄ alkyl, C₁-C₄ alkoxy, C₁-C₄ haloalkyl, C₁-C₄ haloalkoxy, amino, C₁-C₄ alkylamino, C₂-C₈ dialkylamino, CN, NO₂, and OH;
each R^{A3} is independently selected from H, C₁-C₄ alkyl, C₁-C₄ alkoxy, C₁-C₄ haloalkyl, C(O)R^{z}, and C(O)OR^{z}, wherein said C₁-C₄ alkyl is optionally substituted by phenyl, C₁-C₄ alkoxy, C₁-C₄ haloalkoxy, CN, NO₂, or OH;
R is H, C₁-C₄ alkyl, or phenyl;
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Z is O, CH₂, or NH;
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.
C₂. A compound of Formula I,

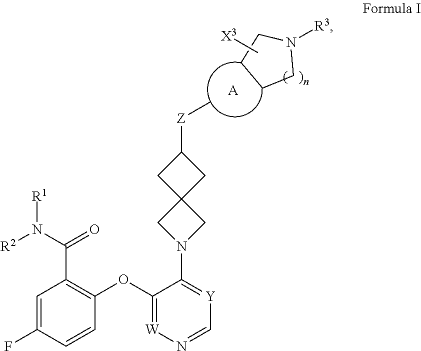

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a})(R^{3b′}), OR^{3a′} or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Z is O, CH₂, or NH;
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR⁴, oxo, CN, C(═O)N(R^{4a′})(R^{4b′}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R¹ is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.
C3. A compound of Formula I-a,

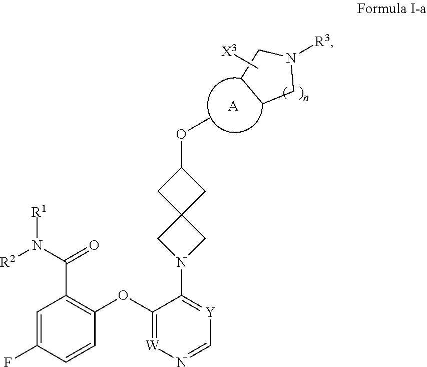

a stereoisomer, or a pharmaceutically acceptable salt thereof, wherein:
W is N or CH;
Y is N or CH;
R¹ is
   (a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
   each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′},
   (b) 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
   (c) C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR⁴, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′}, C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.
C4. The compound of any one of embodiments C1-C3, wherein W is N.
C5. The compound of any one of embodiments C1-C4, wherein W is CH.
C6. The compound of any one of embodiments C1-C5, wherein Y is N.
C7. The compound of any one of embodiments C1-C6, wherein Y is CH.
C8. The compound of any one of embodiments C1-C7, wherein R² is C₁-C₆ alkyl optionally substituted with one or more halo, OH, oxo, CN, or N(R^{4a′})(R^{4b′}).
C9. The compound of any one of embodiments C1-C8, wherein R² is isopropyl.
C10. The compound of any one of embodiments C1-C9, wherein Z is O.
C11. The compound of any one of embodiments C1-C10, wherein R¹ is —C₁-C₆ alkylene-N(R^{3a′})(R^{3b′}) wherein the C₁-C₆ alkylene is optionally substituted with one or more R^{1as};
   each R^{1as} is independently halo, oxo, C₁-C₆ alkyl, C1-C6 haloalkyl, C(═O)(R^{3a′}), N(R^{3a′})(R^{3b′}), or S(═O)₂R^{3a}.
C12. The compound of embodiment C11, wherein R¹ is

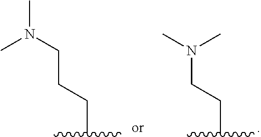

C13. The compound of any one of embodiments C1-C10, wherein R¹ is —C₃-C₁₂ cycloalkylene-N(R^{3a′})(R^{3b′}), wherein the C₃-C₁₂ cycloalkylene is optionally substituted with one or more R^{1as};
   each R^{1as} is independently halo, oxo, C₁-C₆ alkyl, C1-C6 haloalkyl, C(═O)(R^{3a′}), N(R^{3a′})(R^{3b′}), or S(═O)₂R^{3a}.
C14. The compound of embodiment C13, wherein R¹ is

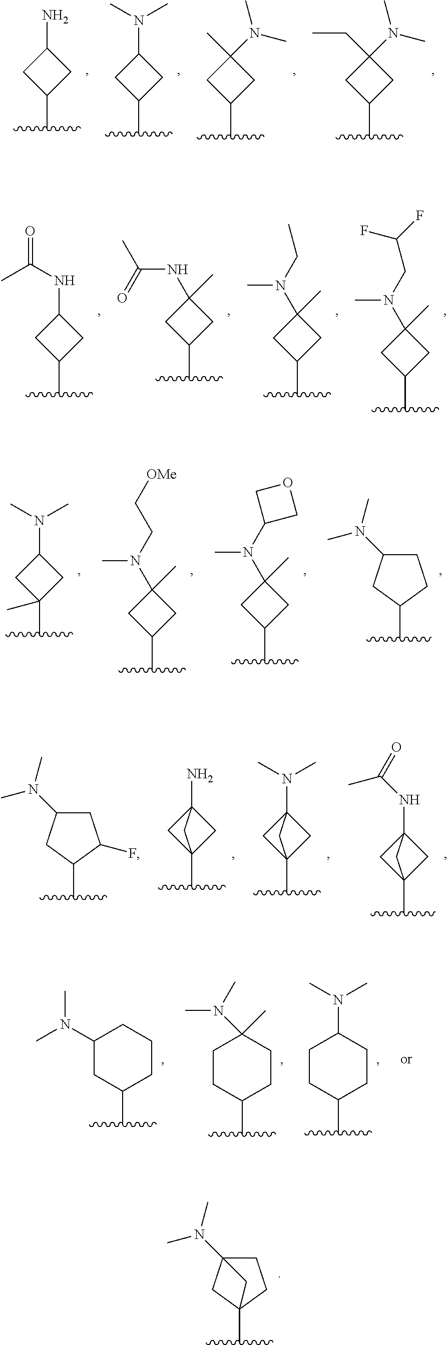

C15. The compound of any one of embodiments C1-C10, wherein R¹ is 3- to 12-membered heterocyclyl which has at least one nitrogen and optionally one or more other heteroatom ring members, wherein the 3- to 12-membered heterocyclyl is optionally substituted with one or more R^{1bs};
   each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}.
C16. The compound of embodiment C15, wherein R¹ is

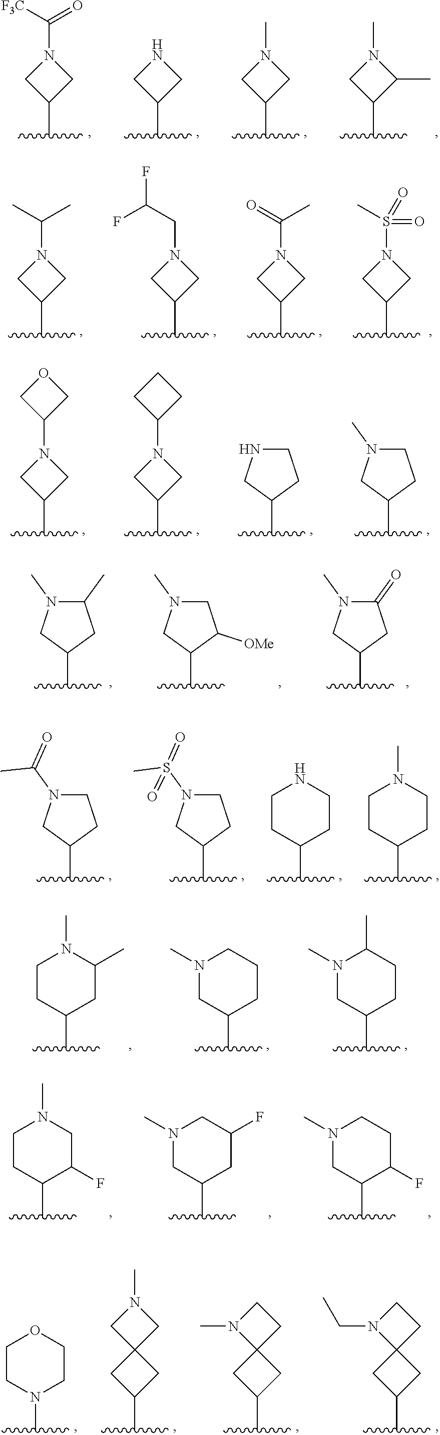

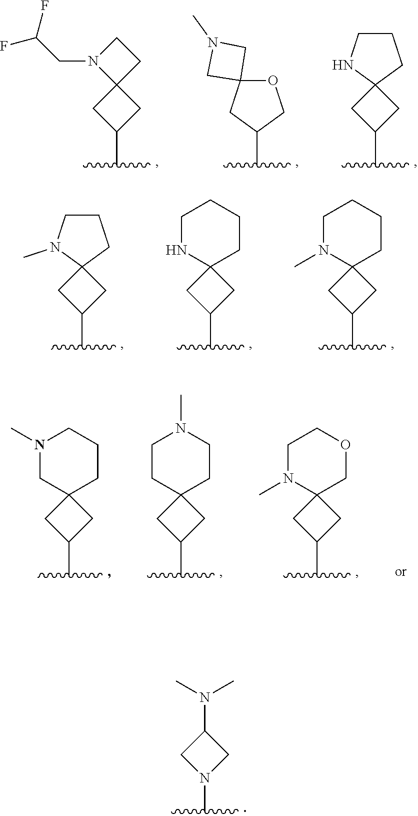

C17. The compound of any one of embodiments C1-C10, wherein R¹ is C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
   each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy.
C18. The compound of embodiment C17, wherein R¹ is

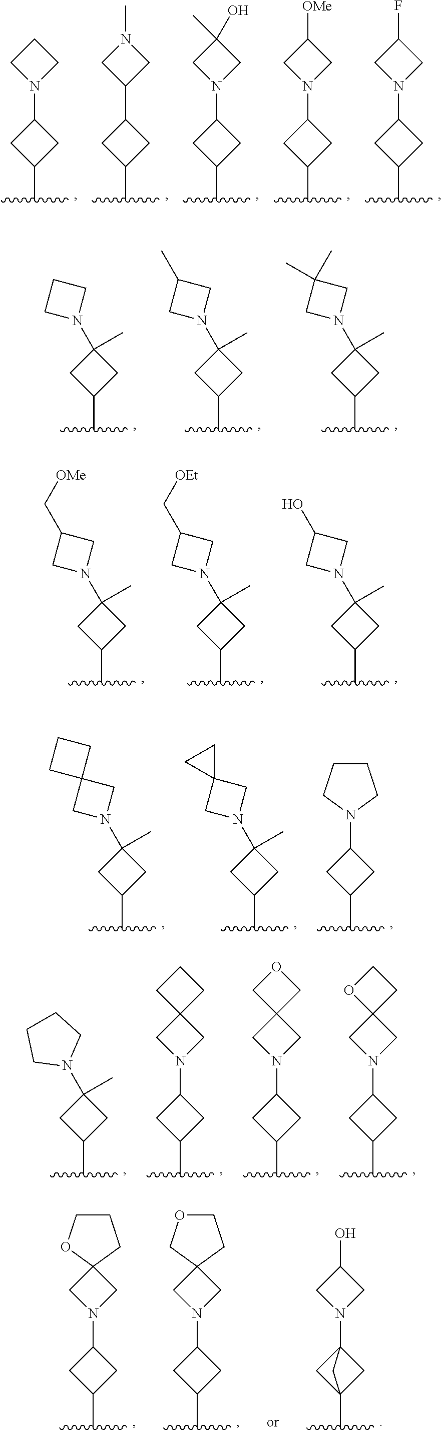

C19. The compound of any one of embodiments C1-C18, wherein Ring A is 6- to 10-membered aryl or 6- to 10-membered heteroaryl.
C20. The compound of any one of embodiments C1-C19, wherein Ring A is

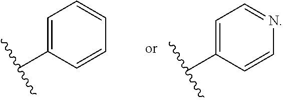 [SMILES: *c1ccccc1 ; *c1ccncc1]

C21. The compound of any one of embodiments C1-C20, wherein

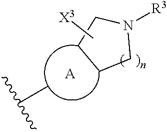

is

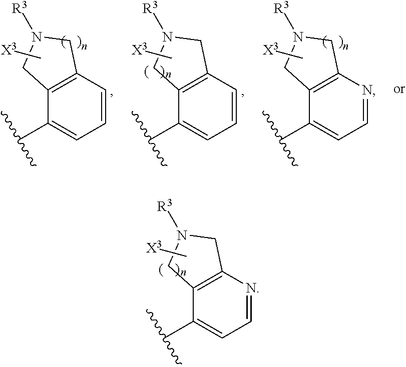

C22. The compound of embodiment C21, wherein

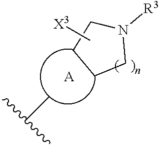

is

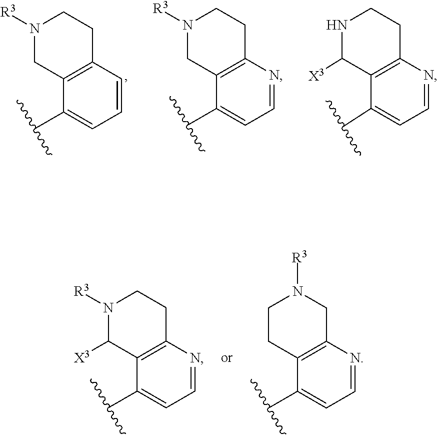

C23. The compound of embodiment C21, wherein

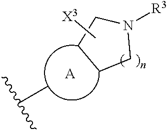

is

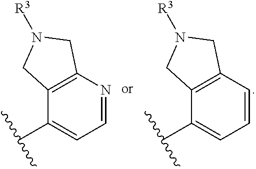

C24. The compound of embodiment C21, wherein

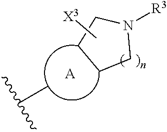

is

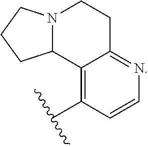 [SMILES: *c1ccnc2c1C1CCCN1CC2]

C25. The compound of any one of embodiments C1-C22, wherein X³ is H or C₁-C₆ alkyl.
C26. The compound of any one of embodiments C1-C25, wherein R³ is C₁-C₆ alkyl optionally substituted with C₆-C₁₀ aryl, 3- to 12-membered heterocyclyl, or OR^{4a}, and R^{4a} is H, CN, or C₁-C₆ alkyl.
C27. The compound of any one of embodiments C1-C26, wherein R³ is methyl, ethyl, isopropyl or tert-butyl.
C28. The compound of any one of embodiments C1-C27, wherein R³ is

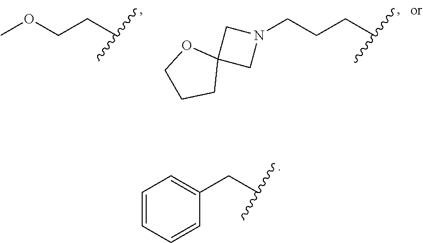

C29. The compound of any one of embodiments C1-C25, wherein R³ is C(O)(R^{4a}) or C(O)O—C(R^{4a})₂—OC(O)(R^{4a}), wherein:
   each R^{4a} is independently H, CN, or C₁-C₆ alkyl optionally substituted with C₆-C₁₀ aryl or N(R^{4a′})(R^{4b′}); and
   each R^{4a′} and R^{4b′} is independently H, C₁-C₆ alkyl, C₃-C₁₂ cycloalkyl, or 3- to 12-membered heterocyclyl.
C30. The compound of any one of embodiments C1-C25 and C29, wherein R³ is

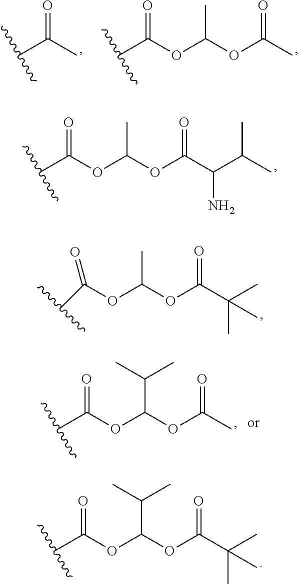

C31. The compound of any one of embodiments C1-C25, wherein R³ is H.
C32. The compound of any one of embodiments C1-C31, wherein n is 1.
C33. The compound of any one of embodiments C1-C31, wherein n is 2.
C34. A compound as shown in Table 1 or a stereoisomer thereof, or a pharmaceutically acceptable salt thereof.
C35. A compound as shown in Table 1 or a pharmaceutically acceptable salt thereof.
C36. A compound as shown in Table 1.
C37. A compound as shown in Table 1 or a pharmaceutically acceptable salt thereof, wherein the salt is hydrochloride.
C38. The compound according to any one of embodiments C1-C37, wherein the compound is useful for the treatment of cancer and wherein the compound minimizes hERG binding.
C39. A pharmaceutical composition comprising a compound of any one of embodiments C1-C38, or a pharmaceutically acceptable salt thereof, and at least one pharmaceutically acceptable carrier.
C40. A pharmaceutical composition comprising a salt or crystalline form of the compound of any one of embodiments C1-C38, and at least one pharmaceutically acceptable carrier.
C41. A method of inhibiting the interaction between menin and MLL comprising contacting the menin and MLL with a compound of any one of embodiments C1-C38 or the pharmaceutical composition of embodiment C39 or embodiment C40.
C42. A method of treating cancer in a patient comprising administering to the patient a compound of any one of embodiments C1-C38 or the pharmaceutical composition of embodiment C39 or embodiment C40.
C43. The method of embodiment C42, wherein the cancer is a hematological cancer.
C44. The method of embodiment C42, wherein the cancer is a leukemia or a lymphoma.
C45. The method of embodiment C42, wherein the cancer is mixed lineage leukemia (MLL), MLL-related leukemia, MLL-associated leukemia, MLL-positive leukemia, MLL-induced leukemia, rearranged mixed lineage (KMT2A-rearranged)leukemia (MLL-r), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL), acute myeloid leukemia (AML), acute granulocytic leukemia, acute nonlymphocytic leukemia, chronic lymphocytic leukemia (CLL), chronic myelogenous leukemia (CML), therapy related leukemia, myelodysplastic syndrome (MDS), myeloproliferative disease (MPD), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sezary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non Hodgkin's lymphoma (malignant lymphoma), or Waldenstrom's macroglobulinemia.
C46. The method of embodiment C42, wherein the cancer is an abstract nucleophosmin (NPM1)-mutated acute myeloid leukemia (i.e., NPM1^{mut} acute myeloid leukemia) or a rearranged mixed lineage (KMT2A-rearranged) leukemia (MLL-r).
C47. The compound of any one of embodiments C1-C38 or a pharmaceutically acceptable salt thereof, or the pharmaceutical composition of embodiment C39 or embodiment C40, for use in treating or preventing a disease caused by, or associated with, menin expression, activity, and/or function.
C48. The compound of any one of embodiments C1-C38 or a pharmaceutically acceptable salt thereof, or the pharmaceutical composition of embodiment C39 or embodiment C40, for use in treating or preventing cancer.
C49. Use of the compound of any one of embodiments C1-C38 or a pharmaceutically acceptable salt thereof, or the pharmaceutical composition of embodiment C39 or embodiment C40, for treating or preventing a disease caused by, or associated with, menin expression, activity, and/or function.
C50. Use of the compound of any one of embodiments C1-C38 or a pharmaceutically acceptable salt thereof, or the pharmaceutical composition of embodiment C39 or embodiment C40, in the manufacture of a medicament for treating or preventing a disease caused by, or associated with, menin expression, activity, and/or function.
C51. Use of a compound of any of embodiments C1-C38 or a pharmaceutically acceptable salt thereof, or the pharmaceutical composition of embodiment C39 or embodiment C40, for treating or preventing cancer.
C52. Use of the compound of any one of embodiments C1-C38 or a pharmaceutically acceptable salt thereof, or the pharmaceutical composition of embodiment C39 or embodiment C40, in the manufacture of a medicament for treating or preventing cancer.
C53. A kit comprising the compound of any one of embodiments C1-C38 or a pharmaceutically acceptable salt thereof, or the pharmaceutical composition of embodiment C39 or embodiment
C40 and instructions for its use.
C54. A method of preparing a compound of any one of embodiments C1-C38 or a pharmaceutically acceptable salt thereof, according to a scheme of the present disclosure or synthetic description in the Examples.
C55. An intermediate useful in the preparation of a compound of any one of embodiments C1-C38 or a pharmaceutically acceptable salt thereof.

### EXAMPLES

As depicted in the Examples below, the compounds of the present disclosure were prepared by the methods described below. Although the Examples illustrate specific methods, those of skill in the art will recognize that other reasonable approaches might also be utilized to prepare these and related compounds.

| Abbreviation | Definition |
|---|---|
| ACN | Acetonitrile |
| AcOH | Acetic acid |
| Ac₂O | Acetic anhydride |
| (Boc)₂O | Di-tert-butyl dicarbonate |
| BOP-Cl | Bis(2-oxo-3-oxazolidinyl)phosphinic chloride |
| n-BuOH | n-Butanol |
| t-BuOH | tert-Butanol |
| tBuXphos | [2-Di-tert-butylphosphino-2′,4′,6′-triisopropylbiphenyl] |
| Cbz-Cl | Benzyl chloroformate; benzyl chloridocarbonate |
| CDI | Carbonyldiimidazole |
| CPME | Cyclopentyl methyl ether |
| DBU | 1,8-Diazabicyclo[5.4.0]undec-7-ene |
| DCE | 1,2-Dichloroethane |
| DCM | Dichloromethane (methylene chloride) |
| DEE | Diethyl ether |
| DIBAL-H | Diisobutylaluminum hydride |
| DIEA (DIPEA), | Diisopropylethylamine |
| iPr₂NEt | |
| DMA (DMAc) | Dimethylacetamide |
| DMAP | 4-Dimethylaminopyridine |
| DME | Dimethoxyethane |
| DMF | Dimethylformamide |
| DMSO | Dimethylsulfoxide |
| dppf | 1,1-bis(Diphenylphosphino)ferrocene |
| DSC | Differential scanning calorimetry |
| EDC.HCl | N-Ethyl-N′-(3-dimethylaminopropyl)carbodiimide hydrochloride |
| Et₃N (TEA) | Triethylamine |
| EtOAc | Ethyl acetate |
| EtOH | Ethanol |
| h | Hour(s) |
| HATU | 1-[bis(Dimethylamino)methylene]-1H-1,2,3-triazolo[4,5-b]pyridinium |
| | 3-oxide hexafluorophosphate |
| HBTU | 2-(1H-Benzotriazole-1-yl)-1,1,3,3-tetramethyluronium |
| | hexafluorophosphate |
| HFIP | Hexafluoroisopropanol |
| HOBt | 1-Hydroxybenzonotrazole |
| HPLC | High performance liquid chromatography |
| IPA | Isopropyl alcohol |
| LAH | Lithium aluminum hydride |
| LCMS | Liquid chromatography-mass spectrometry |
| mCPBA | meta-Chloroperoxybenzoic acid |
| Me | Methyl |
| MeI | Methyl iodide |
| MeOH | Methanol |
| MeOTf | Methyl trifluoromethanesulfonate |
| MeMgCl | Methylmagnesium chloride |
| mg | Milligram |
| min | Minute(s) |
| mL | Milliliters |
| mmol | Millimoles |
| MsCl | Methanesulfonyl chloride |
| MTBE | tert-Butyl methyl ether |
| MW | Microwave |
| NCS | N-Chlorosuccinamide |
| NMP | N-Methyl-2-pyrrolidone |
| NOE | Nuclear Overhauser effect |
| PdCl₂(dppf).CH₂Cl₂ | [1,1′-bis(Diphenylphosphino)ferrocene]dichloropalladium(II), |
| | complex with dichloromethane |
| Pd(dppf)Cl₂ | (1, l′-Bis(diphenylphosphino)ferrocene)palladium(II) dichloride |
| Pd₂dba₃ | Tris(dibenzylideneacetone)dipalladium |
| PhMe | Toluene |
| PG | Protecting group |
| PIDA | Phenyliodine(III) diacetate |
| i-PrOH | Isopropyl alcohol |
| ROE | Rotating-frame Overhauser effect |
| RP | Reverse phase |
| RT | Room temperature |
| Rₜ | Retention time |
| RuPhos | 2-Dicyclohexylphosphino-2′,6′-diisopropoxybiphenyl |
| SFC | Supercritical fluid chromatography |
| SPhos Pd Gen 2 | Chloro(2-dicyclohexylphosphino-2′,6′-dimethoxy-1,1′-biphenyl)[2-(2′- |
| | amino-1,1′-biphenyl)]palladium(II) |
| STAB | Sodium triacetoxyborohydride |
| Tf₂O | Trifluoromethanesulfonic anhydride |
| T3P | Propanephosphonic acid anhydride |
| TBAF | Tetra-n-butylammonium fluoride |
| TFA | Trifluoroacetic acid |
| TFAA | Trifluoroacetic anhydride |
| TFE | Trifluoroethanol |
| THF | Tetrahydrofuran |
| TLC | Thin layer chromatography |
| TMEDA | Tetramethylethylenediamine |
| TMSCl | Trimethylsilyl chloride |
| pTsCl (TsCl) | 4-Toluenesulfonyl chloride |
| UHP | Urea hydrogen peroxide |
| UPLC | Ultra-performance liquid chromatography |
| Xantphos | 4,5-bis(Diphenylphosphino)-9,9-dimethylxanthene |
| XPhos | 2-Dicyclohexyphosphino-2′,4′,6′-triisopropyl-1,1′-biphenyl |

### LCMS

Instrument names: Shimadzu LC2020 Nexera Series; Shimadzu MS2020N-Series; Agilent 1290 Infinity II series with 1260 MSD.
Method A: Mobile Phase A: 5 mM NH₄HCO₃ in water, mobile phase B: ACN; Column: Phenomenex Kinetex EVO C18 (50×3.0) mm, 2.6 μm.
Method B: Mobile Phase A: 0.1% FA in water, mobile phase B: 0.05% FA in ACN; Column: ZORBAX ECLIPSE PLUS C18 (50×2.1) mm, 1.8 μm.
Method C: Mobile Phase: A: 0.05% TFA in water, mobile phase B: ACN; Column: ZORBAX ECLIPSE PLUS C18 (50×2.1) mm, 1.8 μm.
Method D: Mobile Phase A: 10 mM NH₄HCO₃ in water, mobile phase B: ACN; Column: Phenomenex Kinetex EVO C18 (50×3.0) mm, 2.6 μm.
Method E: Mobile Phase A: 5 mM NH₄HCO₃ in water, mobile phase B: ACN; Column: X Bridge C8 (50×4.6) mm, 3.5 μm.
Method F: Mobile Phase A: 5 mM NH₄HCO₃ in water, mobile phase B: ACN; Column: Acquity UPLC BEH C18 (50×2.1) mm, 1.7 μm.
Method G: Mobile Phase A: 5 mM NH₄HCO₃ in water, mobile phase B: ACN; Column: X Bridge BEH C18 (50×2.1) mm, 2.5 μm.
Method H: Mobile Phase: A: 0.1% TFA in water, mobile phase B: ACN; Column: ZORBAX ECLIPSE PLUS C18 (50×2.1) mm, 1.8 μm.
Method I: Mobile Phase A: 0.1% FA in water, mobile phase B: 0.05% FA in ACN; Column: X Bridge C18 BEH (150×4.6) mm, 3.5 μm.
Method J: Mobile Phase A: 0.1% FA in water, mobile phase B: 0.05% FA in ACN; Column: X Bridge BEH C18 (50×2.1) mm, 3.5 μm.
Method K: Mobile Phase A: 5 mM NH₄HCO₃ in water, mobile phase B: ACN; Column: X Bridge BEH C18 (150×4.6) mm, 3.5 μm.
Method L: Mobile Phase A: 0.1% HCOOH in water, mobile phase B: 0.1% HCOOH in ACN; Column: ZORBAX ECLIPSE PLUS C18 (50×2.1 mm), 1.8 μm.
Method M: Mobile Phase A: 5 mM NH₄OAc in water, mobile phase B: ACN; Column: Acquity BEH C18 (50×2.1) mm, 1.7 μm.

### HPLC

Instrument names: Shimadzu LC; Prominence-I series instruments as followed using % with UV detection (Maxplot).
Method A: Mobile Phase A: 10 mM NH₄HCO₃ in water, mobile phase B: ACN; Flow Rate: 2.0 mL/min; Column: X-Bridge C18 (150×4.6) mm, 5 μm
Method B: Mobile Phase A: 0.1% TFA in water, mobile phase B: ACN; Flow Rate: 2.0 mL/min; Column: X-Select CSH C18 (150×4.6) mm, 5 μm.
Method C: Mobile Phase A: 10 mM NH₄HCO₃ in water, mobile phase B: ACN; Flow Rate: 1.2 mL/min; Column: X-Bridge C18 (150×4.6) mm, 3.5 μm
Method D: Mobile Phase A: 10 mM NH₄HCO₃ in water, mobile phase B: ACN; Flow Rate: 2.0 mL/min; Column: X-Bridge C8 (150×4.6) mm, 5 μm
Method E: Mobile Phase A: 5 mM NH₄HCO₃ in water, mobile phase B: ACN; Flow Rate: 1.2 ml/min; Column: X-Bridge C18 (150×4.6) mm, 3.5 μm
Method F: Mobile Phase A: 0.1% TFA in water, mobile phase B: ACN; Flow Rate: 1.2 mL/min; Column: X-Bridge C18 (150×4.6) mm, 3.5 μm
Method G: Mobile Phase A: 0.1% TFA in water, mobile phase B: ACN; Flow Rate: 1.2 m/min; Column: Atlantis T3 C18 (150×4.6) mm, 3 μm.
Method H: Mobile Phase A: 0.1% FA in water, mobile phase B: ACN; Flow Rate: 1.2 mL/min; Column: Atlantis T3 C18 (150×4.6) mm, 3 μm.

### Prep HPLC

Method A: Mobile Phase A: 10 mM NH₄HCO₃ in water; mobile phase B: ACN; Flow Rate: 18.0 mL/min; Column: X-Timate C18 (250×21.2) mm, 5 μm.
Method B: Mobile Phase A: 10 mM NH₄HCO₃ in water; mobile phase B: ACN; Flow Rate: 15.0 mL/min; Column: X-Select CSH C18 (250×19) mm, 5 μm.
Method C: Mobile Phase A: 10 mM NH₄HCO₃ in water; mobile phase B: ACN; Flow Rate: 15.0 m/min; Column: SHIMPACK GIST C18 (150×19.0) mm, 5 μm.
Method D: Mobile Phase A: 10 mM NH₄HCO₃ in water, mobile phase B: ACN; Flow Rate: 18.0 mL/min; Column: SHIMPACK GIST C18 (150×19.0) mm, 5 μm.
Method E: Mobile Phase A: 5 mM NH₄HCO₃ in water; mobile phase B: ACN; Flow Rate: 18.0 mL/min; Column: X-TIMATE C18 (250×21.2) mm, 5 μm.
Method F: Mobile Phase A: 0.1% FA in water; mobile phase B: ACN; Flow Rate: 18.0 mL/min; Column: X-Timate C18 (250×21.2) mm, 5 μm
Method G: Mobile Phase A: 0.1% TFA in water; mobile phase B: ACN; Flow Rate: 15.0 mL/min; Column: X-Select CSH C18 (150×19) mm, 5 μm.
Method H: Mobile Phase A: 10 mM NH₄HCO₃ in water; mobile phase B: ACN; Flow Rate: 15.0 mL/min; Column: X-Select CSH C18 (150×19) mm, 5 μm.
Method I: Mobile Phase A: 10 mM NH₄HCO₃ in water; mobile phase B: ACN; Flow Rate: 12.0 mL/min; Column: X-Bridge OBD C8 (150×19.0) mm, 5 μm.
Method J: Mobile Phase A: 10 mM NH₄HCO₃ in water; mobile phase B: ACN; Flow Rate: 18.0 mL/min; Column: SHIMPACK GIST C18 (150×20.0) mm, 5 μm.
Method K: Mobile Phase A: 10 mM NH₄HCO₃ in water; mobile phase B: ACN; Flow Rate: 15.0 mL/min; Column: X-Select CSH C18 (150×19) mm, 5 μm.
Method L: Mobile Phase A: 10 mM NH₄HCO₃ in water; mobile phase B: ACN; Flow Rate: 15.0 m/min; Column: X-Bridge OBD C8 (150×19.0) mm, 5 μm.
Method M: Mobile Phase A: 5 mM NH₄HCO₃ in water, mobile phase B: ACN; Flow Rate: 18.0 mL/min; Column: Gemini NX C18 (250×21.2) mm, 5 μm.
Method N: Mobile Phase A: 0.1% FA in water, mobile phase B: ACN; Flow Rate: 13 mL/min; Column: Atlantis T3 C18 (250×19) mm, 5 μm.
Method O: Mobile Phase A: 10 mM NH₄HCO₃ in water; mobile phase B: ACN; Flow Rate: 15.0 mL/min; Column: X-Timate C18 (250×21.2) mm, 5 μm.
Method P: Mobile Phase A: 10 mM NH₄HCO₃ in water; mobile phase B: ACN; Flow Rate: 18.0 mL/min; Column: Gemini C18 (250×21.2) mm, 5 μm.

### Chiral SFC

### Instrument Names: Shimadzu Nexera UC SFC Analytical; PIC-SFC Analytical-10

Method A: Mobile Phase A: CO₂; Co-solvent—0.5% Isopropylamine in [ACN:MeOH (1:1)](55:45); Flow Rate: 4.0 mL/min; % Co-Solvent: 45%; Column: Whelk-(R,R) (250×4.6) mm, 5 μm.
Method B: Mobile Phase A: CO₂; Co-solvent—0.5% Isopropylamine in MeOH (70:30); Flow Rate: 4.0 mL/min; % Co-Solvent: 30%; Column: I-Cellulose Z (250×4.6) mm, 5 μm.
Method C: Mobile Phase A: CO₂; Co-solvent—0.5% Isopropylamine in MeOH (50:50); Flow Rate: 4.0 mL/min; % Co-Solvent: 50%; Column: I-Cellulose Z (250×4.6) mm, 5 μm.
Method D: Mobile Phase A: CO₂; Co-solvent—0.5% Isopropylamine in MeOH (75:25); Flow Rate: 4.0 mL/min; % Co-Solvent: 25%; Column: I-Cellulose B (250×4.6) mm, 5 μm.
Method E: Mobile Phase A: CO₂; Co-solvent—0.5% Isopropylamine in IPA (50:50); Flow Rate: 4.0 mL/min; % Co-Solvent: 50%; Column: LUX-I-Amylose 3 (250×4.6) mm, 5 μm.
Method F: Mobile Phase A: CO₂; Co-solvent—0.5% Isopropylamine in MeOH (65:35); Flow Rate: 4.0 mL/min; % Co-Solvent: 35%; Column: C Cellulose B (250×4.6) mm, 5 μm.
Method G: Mobile Phase A: CO₂; Co-solvent—0.5% Isopropylamine in MeOH (50:50); Flow Rate: 4.0 mL/min; % Co-Solvent: 50%; Column: LUX-I-Amylose3 (250×4.6) mm, 5 μm.
Method H: Mobile Phase A: CO₂; Co-solvent—0.5% Isopropylamine in MeOH (50:50); Flow Rate: 4.0 mL/min; % Co-Solvent: 50%; Column: Whelk-(R,R) (250×4.6) mm, 5 μm.
Method I: Mobile Phase A: CO₂; Co-solvent—0.5% Isopropylamine in MeOH (60:40); Flow Rate: 4.0 mL/min; % Co-Solvent: 40%; Column: Whelk-(R,R) (250×4.6) mm, 5 μm.
Method J: Mobile Phase A: CO₂; Co-solvent—0.5% Isopropylamine in MeOH (55:45); Flow Rate: 4.0 mL/min; % Co-Solvent: 45%; Column: Whelk-(RR) (250×4.6) mm, 5 μm.
Method K: Mobile Phase A: CO₂; Co-solvent—0.5% Isopropylamine in [ACN:MeOH (1:1)](50:50); Flow Rate: 4.0 mL/min; % Co-Solvent: 50%; Column: I-Cellulose Z (250×4.6) mm, 5 μm.
Method L: Mobile Phase A: CO₂; Co-solvent—0.5% Isopropylamine in MeOH (70:30); Flow Rate: 4.0 mL/min; % Co-Solvent: 30%; Column: I Cellulose B (250×4.6) mm
Method M: Mobile Phase A: CO₂; Co-solvent—0.5% Isopropylamine in [ACN:MeOH (1:1)](70:30); Flow Rate: 4.0 mL/min; % Co-Solvent: 30%; Column: I Cellulose B (250×4.6) mm, 5 μm.
Method N: Mobile Phase A: CO₂; Co-solvent—0.5% Isopropylamine in MeOH (60:40); Flow Rate: 4.0 mL/min; % Co-Solvent: 40%; Column: I Cellulose B (250×4.6) mm, 5 μm.
Method O: Mobile Phase A: CO₂; Co-solvent—0.5% Isopropylamine in [ACN:MeOH (1:1)](60:40); Flow Rate: 4.0 mL/min; % Co-Solvent: 40%; Column: I-Amylose A (250×4.6) mm, 5 μm.
Method P: Mobile Phase A: CO₂; Co-solvent—0.5% Isopropylamine in [ACN:MeOH (1:1)](55:45); Flow Rate: 4.0 mL/min; % Co-Solvent: 45%; Column: I-Cellulose Z (250×4.6) mm, 5 μm.
Method Q: Mobile Phase A: CO₂; Co-solvent—0.5% Isopropylamine in MeOH (55:45); Flow Rate: 5.0 mL/min; % Co-Solvent: 45%; Column: I-Cellulose Z (250×4.6) mm, 5 μm.
Method R: Mobile Phase A: CO₂; Co-solvent—0.5% Isopropylamine in MeOH (65:35); Flow Rate: 4.0 mL/min; % Co-Solvent: 35%; Column: CHIRALPAK-IH (250×4.6) mm, 5 μm.
Method S: Mobile Phase A: CO₂; Co-solvent—0.5% Isopropylamine in MeOH (55:45); Flow Rate: 5.0 mL/min; % Co-Solvent: 45%; Column: Whelk-(RR) (250×4.6) mm, 5 μm.
Method T: Mobile Phase A: CO₂; Co-solvent—0.5% Isopropylamine in [ACN:MeOH (1:1)](50:50); Flow Rate: 4.0 mL/min; % Co-Solvent: 50%; Column: I-Cellulose Z (250×4.6) mm, 5 μm.
Method U: Mobile Phase A: CO₂; Co-solvent—0.5% Isopropylamine in MeOH (55:45); Flow Rate: 4.0 mL/min; % Co-Solvent: 45%; Column: I Cellulose B (250×4.6) mm
Method V: Mobile Phase A: CO₂; Co-solvent—0.5% Isopropylamine in MeOH (60:40); Flow Rate: 4.0 mL/min; % Co-Solvent: 40%; Column: LUX-I-Amylose-3 (250×4.6) mm, 5 μm.
Method W: Mobile Phase A: CO₂; Co-solvent—0.5% Isopropylamine in [ACN:MeOH (1:1)](55:45); Flow Rate: 5.0 mL/min; % Co-Solvent: 45%; Column: LUX-I-Amylose 3 (250×4.6) mm, 5 μm.
Method X: Mobile Phase A: CO₂; Co-solvent—0.5% Isopropylamine in [IPA:MeOH (1:1)](50:50); Flow Rate: 4.0 mL/min; % Co-Solvent: 50%; Column: LUX-Cellulose-4 (250×4.6) mm, 5 μm.
Method Y: Mobile Phase A: CO₂; Co-solvent—0.5% Isopropylamine in MeOH (55:45); Flow Rate: 4.0 mL/min; % Co-Solvent: 45%; Column: I-Cellulose Z (250×4.6) mm, 5 μm.
Method Z: Mobile Phase A: CO₂; Co-solvent—0.5% Isopropylamine in MeOH (55:45); Flow Rate: 4.0 mL/min; % Co-Solvent: 45%; Column: CHIRALPAK-IG (250×4.6) mm, 5 μm.
Method AA: Mobile Phase A: CO₂; Co-solvent—0.5% Isopropylamine in MeOH (65:35); Flow Rate: 4.0 mL/min; % Co-Solvent: 35%; Column: I-Amylose A (250×4.6) mm
Method AB: Mobile Phase A: CO₂; Co-solvent—0.5% Isopropylamine in MeOH (70:30); Flow Rate: 4.0 mL/min; % Co-Solvent: 30%; Column: C Cellulose B (250×4.6) mm, 5 μm.
Method AC: Mobile Phase A: CO₂; Co-solvent—0.5% Isopropylamine in MeOH (65:35); Flow Rate: 4.0 mL/min; % Co-Solvent: 35%; Column: I-Cellulose B (250×4.6) mm, 5 μm.
Method AD: Mobile Phase A: CO₂; Co-solvent—0.5% Isopropylamine in MeOH (50:50); Flow Rate: 5.0 mL/min; % Co-Solvent: 50; Column: I-Cellulose Z (250×4.6) mm, 5 μm.
Method AE: Mobile Phase A: CO₂; Co-solvent—0.5% Isopropylamine in IPA (70:30); Flow Rate: 4.0 mL/min; % Co-Solvent: 30%; Column: LUX-Amylose-1 (250×4.6) mm, 5 μm.
Method AF: Mobile Phase A: CO₂; Co-solvent—0.5% Isopropylamine in MeOH (50:50); Flow Rate: 5.0 mL/min; % Co-Solvent: 45%; Column: Whelk-(R,R) (250×4.6) mm, 5 μm.
Method AG: Mobile Phase A: CO₂; Co-solvent—0.5% Isopropylamine in IPA (60:40); Flow Rate: 4.0 mL/min; % Co-Solvent: 40%; Column: LUX-I-Amylose-3 (250×4.6) mm, 5 μm.
Method AH: Mobile Phase A: CO₂; Co-solvent—0.5% Isopropylamine [IPA:ACN (1:1)](55:45); Flow Rate: 4.0 mL/min; % Co-Solvent: 45%; Column: CHIRALPAK-IK (250×4.6) mm, 5 μm.
Method AL: Mobile Phase A: CO₂; Co-solvent—0.5% Isopropylamine in [ACN:MeOH (1:1)](55:45); Flow Rate: 5.0 mL/min; % Co-Solvent: 45%; Column: Whelk-(R,R) (250×4.6) mm, 5 μm.
Method AJ: Mobile Phase A: CO₂; Co-solvent—0.5% Isopropylamine in [IPA:ACN (1:1)](50:50); Flow Rate: 4.0 mL/min; % Co-Solvent: 50%; Column: I-Cellulose Z (250×4.6) mm, 5 μm.
Method AK: Mobile Phase A: CO₂; Co-solvent—0.5% Isopropylamine in MeOH (55:45); Flow Rate: 5.0 mL/min; % Co-Solvent: 45%; Column: CHIRALPAK-IK (250×4.6) mm, 5 μm.
Method AL: Mobile Phase A: CO₂; Co-solvent—0.5% Isopropylamine in MeOH (60:40); Flow Rate: 5.0 mL/min; % Co-Solvent: 40%; Column: I Cellulose B (250×4.6) mm, 5 μm.
Method AM: Mobile Phase A: CO₂; Co-solvent—0.5% Isopropylamine in MeOH (60:40); Flow Rate: 5.0 mL/min; % Co-Solvent: 40%; Column: Whelk-(R,R) (250×4.6) mm, 5 μm.
Method AN: Mobile Phase A: CO₂; Co-solvent—0.5% Isopropylamine in [ACN:MeOH (1:1)](55:45); Flow Rate: 5.0 mL/min; % Co-Solvent: 45%; Column: LUX-I-Amylose 3 (250×4.6) mm, 5 μm.
Method AO: Mobile Phase A: CO₂; Co-solvent—0.5% Isopropylamine in MeOH (80:20); Flow Rate: 5.0 mL/min; % Co-Solvent: 20%; Column: CHIRALPAK AS-H (250×4.6) mm, 5 μm.
Method AP: Mobile Phase A: CO₂; Co-solvent—0.5% Isopropylamine in MeOH (70:30); Flow Rate: 5.0 mL/min; % Co-Solvent: 30%; Column: CHIRALPAK AS-H (250×4.6) mm, 5 μm.
Method AQ: Mobile Phase A: CO₂; Co-solvent—0.5% Isopropylamine in [ACN:MeOH (1:1)](50:50); Flow Rate: 5.0 mL/min; % Co-Solvent: 50%; Column: Whelk-(R,R) (250×4.6) mm, 5 μm.
Method AR: Mobile Phase A: CO₂; Co-solvent—0.5% Isopropylamine in IPA (50:50); Flow Rate: 5.0 mL/min; % Co-Solvent: 50%; Column: LUX-I-Amylose 3 (250×4.6) mm, 5 μm.
Method AS: Mobile Phase A: CO₂; Co-solvent—0.5% Isopropylamine in [ACN:MeOH (1:1)](60:40); Flow Rate: 5.0 mL/min; % Co-Solvent: 40%; Column: Whelk-(R,R) (250×4.6) mm, 5 μm.
Method AT: Mobile Phase A: CO₂; Co-solvent—0.5% Isopropylamine in [IPA:ACN (1:1)](50:50); Flow Rate: 4.0 mL/min; % Co-Solvent: 50%; Column: CHIRALPAK-IK (250×4.6) mm, 5 μm.
Method AU: Mobile Phase A: CO₂; Co-solvent—0.5% Isopropylamine in [IPA:ACN (1:1)](50:50); Flow Rate: 4.0 mL/min; % Co-Solvent: 50%; Column: I-Cellulose C (250×4.6) mm, 5 μm.
Method AV: Mobile Phase A: CO₂; Co-solvent—0.5% Isopropylamine [IPA:ACN (1:1)](50:50); Flow Rate: 4.0 mL/min; % Co-Solvent: 50%; Column: Whelk-(R,R) (250×4.6) mm, 5 μm.
Method AW: Mobile Phase A: CO₂; Co-solvent—0.5% Isopropylamine in IPA (55:45); Flow Rate: 4.0 mL/min; % Co-Solvent: 45%; Column: LUX-I-Amylose 3 (250×4.6) mm, 5 μm.
Method AX: Mobile Phase A: CO₂; Co-solvent—0.5% Isopropylamine in IPA (50:50); Flow Rate: 4.0 mL/min; % Co-Solvent: 50%; Column: CHIRALPAK-IK (250×4.6) mm, 5 μm.
Method AY: Mobile Phase A: CO₂; Co-solvent—0.5% Isopropylamine in [MeOH:ACN (1:1)](60:40); Flow Rate: 4.0 mL/min; % Co-Solvent: 40%; Column: LUX-Cellulose 4 (250×4.6) mm, 5 μm.
Method AZ: Mobile Phase A: CO₂; Co-solvent—0.5% Isopropylamine in MeOH (50:50); Flow Rate: 4.0 mL/min; % Co-Solvent: 50%; Column: LUX-Cellulose 4 (250×4.6) mm, 5 μm.
Method AAA: Mobile Phase A: CO₂; Co-solvent—0.5% Isopropylamine in [IPA:ACN (1:1)](55:45); Flow Rate: 4.0 mL/min; % Co-Solvent: 45%; Column: I-Cellulose Z (250×4.6) mm, 5 μm.
Method AAB: Mobile Phase A: CO₂; Co-solvent—0.5% Isopropylamine in MeOH (75:25); Flow Rate: 4.0 mL/min; % Co-Solvent: 25%; Column: I Cellulose J (250×4.6) mm, 5 μm.
Method AAC: Mobile Phase A: CO₂; Co-solvent—0.5% Isopropylamine in MeOH (55:45); Flow Rate: 4.0 mL/min; % Co-Solvent: 45%; Column: C Cellulose B (250×4.6) mm, 5 μm.
Method AAD: Mobile Phase A: CO₂; Co-solvent—0.5% Isopropylamine in MeOH (55:45); Flow Rate: 5.0 mL/min; % Co-Solvent: 50%; Column: LUX-I-Amylose 2 (250×4.6) mm, 5 μm.
Method AAE: Mobile Phase A: CO₂; Co-solvent—0.5% Isopropylamine in MeOH (55:45); Flow Rate: 4.0 mL/min; % Co-Solvent: 50%; Column: LUX-Cellulose-4 (250×4.6) mm, 5 μm.
Method AAF: Mobile Phase A: CO₂; Co-solvent—0.5% Isopropylamine in MeOH (60:40); Flow Rate: 4.0 mL/min; % Co-Solvent: 40%; Column: I-Cellulose Z (250×4.6) mm, 5 μm.
Method AAG: Mobile Phase A: CO₂; Co-solvent—0.5% Isopropylamine in [ACN:MeOH (1:1)](55:45); Flow Rate: 4.0 mL/min; % Co-Solvent: 45%; Column: I Cellulose B (250×4.6) mm, 5 μm.
Method AAH: Mobile Phase A: CO₂; Co-solvent—0.5% Isopropylamine in [ACN:MeOH (1:1)](45:55); Flow Rate: 5.0 mL/min; % Co-Solvent: 45%; Column: Whelk-(R,R) (250×4.6) mm, 5 μm.
Method AAI: Mobile Phase A: CO₂; Co-solvent—0.5% Isopropylamine in IPA (50:50); Flow Rate: 4.0 mL/min; % Co-Solvent: 50%; Column: I Cellulose B (250×4.6) mm, 5 μm.
Method AAJ: Mobile Phase A: CO₂; Co-solvent—0.5% Isopropylamine in MeOH (60:40); Flow Rate: 4.0 mL/min; % Co-Solvent: 40%; Column: CHIRALPAK AS-H (250×4.6) mm, 5 μm.
Method AAK: Mobile Phase A: CO₂; Co-solvent—0.5% Isopropylamine in MeOH (80:20); Flow Rate: 4.0 mL/min; % Co-Solvent: 20%; Column: I-Cellulose C (250×4.6) mm, 5 m.
Method AAL: Mobile Phase A: CO₂; Co-solvent—0.5% Isopropylamine in MeOH (60:40); Flow Rate: 4.0 mL/min; % Co-Solvent: 40%; Column: LUX-Cellulose 4 (250×4.6) mm, 5 μm.
Method AAM: Mobile Phase A: CO₂; Co-solvent—0.5% Isopropylamine in MeOH (50:50); Flow Rate: 4.0 mL/min; % Co-Solvent: 50%; Column: LUX-Cellulose 4 (250×4.6) mm, 5 μm.
Method AAN: Mobile Phase A: CO₂; Co-solvent—0.5% Isopropylamine in [IPA:ACN (1:1)](60:40); Flow Rate: 4.0 mL/min; % Co-Solvent: 40%; Column: I-Cellulose Z (250×4.6) mm, 5 μm.
Method AAO: Mobile Phase A: CO₂; Co-solvent—0.5% Isopropylamine in MeOH (85:15); Flow Rate: 4 mL/min; % Co-Solvent: 15%; Column: I-Cellulose Z (250×4.6) mm, 5 μm.
Method AAP: Mobile Phase A: CO₂; Co-solvent—0.5% Isopropylamine in [MeOH:ACN (1:1)](60:40); Flow Rate: 4.0 mL/min; % Co-Solvent: 40%; Column: LUX-Amylose-1 (250×4.6) mm, 5 μm.
Method AAQ: Mobile Phase A: CO₂; Co-solvent—0.5% Isopropylamine in [ACN:MeOH (1:1)](55:45); Flow Rate: 4.0 mL/min; % Co-Solvent: 45%; Column: I-Amylose A (250×4.6) mm, 5 μm.
Method AAR: Mobile Phase A: CO₂; Co-solvent—0.5% Isopropylamine in MeOH (60:40); Flow Rate: 4.0 mL/min; % Co-Solvent: 40%; Column: I-Cellulose C (250×4.6) mm, 5 μm.
Method AAS: Mobile Phase A: CO₂; Co-solvent—0.5% Isopropylamine in [ACN:MeOH (1:1)](55:45); Flow Rate: 4.0 mL/min; % Co-Solvent: 45%; Column: CHIRALPAK-U (250×4.6) mm, 5 μm.
Method AAT: Mobile Phase A: CO₂; Co-solvent—0.5% Isopropylamine in IPA (50:50); Flow Rate: 4.0 mL/min; % Co-Solvent: 50%; Column: CHIRALPAK-IG (250×4.6) mm, 5 μm.
Method AAU: Mobile Phase A: CO₂; Co-solvent—0.5% Isopropylamine in IPA (85:15); Flow Rate: 4.0 mL/min; % Co-Solvent: 15%; Column: Whelk-(R,R) (250×4.6) mm, 5 μm. Chiral Prep SFC
Method A: Mobile Phase A: CO₂; Co-solvent—0.5% Isopropylamine in [IPA:ACN (1:1) (50:50)]; Flow Rate: 100 mL/min; % Co-Solvent: 50%; Column: I-Cellulose Z (250×30) mm, 5 sm.
Method B: Mobile Phase A: CO₂; Co-solvent—0.5% Isopropylamine in MeOH (70:30); Flow Rate: 100 mL/min; % Co-Solvent: 30%; Column: I-Cellulose Z (250×30) mm, 5 μm.
Method C: Mobile Phase A: CO₂; Co-solvent—0.5% Isopropylamine in IPA (50:50); Flow Rate: 60 mL/min; % Co-Solvent: 50%; Column: LUX-I Amylose 3 (250×21) mm, 5 μm.
Method D: Mobile Phase A: CO₂: Co-solvent—0.5% Isopropylamine in MeOH (55:45); Flow Rate: 60 mL/min; % Co-Solvent: 45%; Column: RR WHELK (250×21) mm, 5 μm
Method E: Mobile Phase A: CO₂; Co-solvent—0.5% Isopropylamine in [IPA:MeOH (1:1)](50:50); Flow Rate: 60 m/min; % Co-Solvent: 50%; Column: LUX-Cellulose-4 (250×21) mm, 5 μm.
Method F: Mobile Phase A: CO₂; Co-solvent—0.5% Isopropylamine in MeOH (50:50); Flow Rate: 100 mL/min; % Co-Solvent: 50%; Column: I-Cellulose Z (250×30) mm, 5 μm.
Method G: Mobile Phase A: CO₂; Co-solvent—0.5% Isopropylamine in IPA (70:30); Flow Rate: 60 mL/min; % Co-Solvent: 30%; Column: C Cellulose B (250×21) mm, 5 μm.
Method H: Mobile Phase A: CO₂; Co-solvent—0.5% Isopropylamine in IPA (50:50); Flow Rate: 100 m/min; % Co-Solvent: 50%; Column: LUX-I Amylose 3 (250×30) mm, 5 μm.
Method I: Mobile Phase A: CO₂: Co-solvent 0.5% Isopropylamine in IPA (70:30); Flow Rate: 60 mL/min; % Co-Solvent: 30%; Column: LUX A1 (250×21) mm, 5 μm.
Method J: Mobile Phase A: CO₂; Co-solvent—0.5% Isopropylamine in IPA (70:30); Flow Rate: 50 mL/min; % Co-Solvent: 30%; Column: LUX-Cellulose-1 (250×21) mm, 5 μm.
Method K: Mobile Phase A: CO₂; Co-solvent—0.5% Isopropylamine in [IPA:ACN (1:1)](55:45); Flow Rate: 100 mL/min; % Co-Solvent: 45%; Column: CHIRALPAK-IK (250×21) mm, 5 μm.
Method L: Mobile Phase A: CO₂; Co-solvent—0.5% Isopropylamine in [IPA:ACN (1:1)](70:30); Flow Rate: 60 mL/min; % Co-Solvent: 30%; Column: I-Cellulose C (250×20) mm, 5 μm.
Method M: Mobile Phase A: CO₂; Co-solvent—0.5% Isopropylamine in IPA (50:50); Flow Rate: 100 mL/min; % Co-Solvent: 50%; Column: LUX-I Amylose 3 (250×30) mm, 5 μm.
Method N: Mobile Phase A: CO₂: Co-solvent—0.5% Isopropylamine in MeOH (50:50); Flow Rate: 100 mL/min; % Co-Solvent: 50%; Column: RR WHELK (250×21) mm, 5 μm
Method O: Mobile Phase A: CO₂; Co-solvent—0.5% Isopropylamine in IPA (50:50); Flow Rate: 120 mL/min; % Co-Solvent: 50%; Column: CHIRALPAK-IK (250×30) mm, 5 μm.
Method P: Mobile Phase A: CO₂; Co-solvent—0.5% Isopropylamine in [ACN:MeOH (1:1) (50:50)]; Flow Rate: 100 mL/min; % Co-Solvent: 50%; Column: I-Cellulose Z (250×30) mm, 5 μm.
Method Q: Mobile Phase A: CO₂; Co-solvent—0.5% Isopropylamine in [MeOH:ACN (1:1)](55:45); Flow Rate: 100 mL/min; % Co-Solvent: 45%; Column: LUX-Cellulose 4 (250×30) mm, 5 μm.
Method R: Mobile Phase A: CO₂; Co-solvent—0.5% Isopropylamine in MeOH (55:45); Flow Rate: 100 mL/min; % Co-Solvent: 45%; Column: LUX-Cellulose 4 (250×30) mm, 5 μm.
Method S: Mobile Phase A: CO₂; Co-solvent—0.5% Isopropylamine in [IPA:ACN (1:1)](55:45); Flow Rate: 100 mL/min; % Co-Solvent: 45%; Column: 1-Cellulose Z (250×30) mm, 5 μm.
Method T: Mobile Phase A: CO₂; Co-solvent—0.5% Isopropylamine in MeOH (82:18); Flow Rate: 60 mL/min; % Co-Solvent: 18%; Column: C Cellulose B (250×20) mm, 5 μm.
Method U: Mobile Phase A: CO₂; Co-solvent—0.5% Isopropylamine in IPA (55:45); Flow Rate: 100 mL/min; % Co-Solvent: 45%; Column: CHIRALPAK-IK (250×21) mm, 5 μm.
Method V: Mobile Phase A: CO₂; Co-solvent—0.5% Isopropylamine in [IPA:ACN (1:1)](50:50); Flow Rate: 100 mL/min; % Co-Solvent: 50%; Column: CHIRALPAK-IK (250×30) mm, 5 μm.
Method W: Mobile Phase A: CO₂; Co-solvent—0.5% Isopropylamine in [IPA:ACN (1:1)](55:45); Flow Rate: 120 mL/min; % Co-Solvent: 45%; Column: I-Cellulose Z (250×30) mm, 5 μm.
Method X: Mobile Phase A: CO₂; Co-solvent 0.2% Isopropylamine in [IPA:ACN (1:1)](50:50); Flow Rate: 100 mL/min; % Co-Solvent: 45%; Column: I-Cellulose Z (250×30) mm, 5 μm.
Method Y: Mobile Phase A: CO₂; Co-solvent—0.5% Isopropylamine in MeOH (80:20); Flow Rate: 300 mL/min, % Co-Solvent: 20%; Column: I-Cellulose C (250×50) mm, 5 μm.
Method Z: Mobile Phase A: CO₂; Co-solvent—0.5% Isopropylamine in MeOH (45:55); Flow Rate: 60 mL/min; % Co-Solvent: 55%; Column: LUX-Cellulose 4 (250×30) mm, 5 μm.
Method AA: Mobile Phase A: CO₂; Co-solvent—0.5% Isopropylamine in MeOH (80:20); Flow Rate: 100 mL/min; % Co-Solvent: 20%; Column: I-Cellulose Z (250×30) mm, 5 μm.
Method AB: Mobile Phase A: CO₂; Co-solvent—0.5% Isopropylamine in MeOH (55:45); Flow Rate: 100 mL/min; % Co-Solvent: 45%; Column: I-Cellulose C (250×30) mm, 5 μm.
Method AC: Mobile Phase A: CO₂; Co-solvent—0.5% Isopropylamine in [ACN:MeOH (1:1)](60:40); Flow Rate: 120 mL/min; % Co-Solvent: 40%; Column: Whelk-(R,R) (250×30) mm, 5 μm.
Method AD: Mobile Phase A: CO₂; Co-solvent—0.5% Isopropylamine in IPA (50:50); Flow Rate: 50 mL/min; % Co-Solvent: 50%; Column: CHIRALPAK-IG (250×20) mm, 5 μm.
Method AE: Mobile Phase A: CO₂; Co-solvent—0.5% Isopropylamine in [ACN:MeOH (1:1) (55:45)]; Flow Rate: 110 mL/min; % Co-Solvent: 45%; Column: I-Cellulose Z (250×30) mm, 5 μm.
Method AF: Mobile Phase A: CO₂; Co-solvent—0.5% Isopropylamine in [ACN:MeOH (1:1) (60:40)]; Flow Rate: 100 mL/min; % Co-Solvent: 40%; Column: 1-Cellulose Z (250×30) mm, 5 μm.
Method AG: Mobile Phase A: CO₂: Co-solvent—0.5% Isopropylamine in IPA (85:15); Flow Rate: 120 mL/min; % Co-Solvent: 15%; Column: RR WHELK (250×21) mm, 5 μm. Achiral SFC Instrument names: PIC-SFC-175; PIC-SFC-400
Method A: Mobile Phase A: CO₂: Co-solvent—0.5% Isopropylamine in MeOH (85:15); Flow Rate: 4.0 mL/min; % Co-Solvent: 15%; Column: DIOL (250×4.6) mm, 5 μm.
Method B: Mobile Phase A: CO₂: Co-solvent—0.5% Isopropylamine in MeOH (85:15); Flow Rate: 4 mL/min; % Co-Solvent: 15%; Column: 4-Ethyl pyridine (250×4.6) mm, 5 μm.
Method C: Mobile Phase A: CO₂: Co-solvent—0.5% Isopropylamine in MeOH (90:10); Flow Rate: 4 mL/min; % Co-Solvent: 10%; Column: Prontosil 4-Ethyl pyridine (250×4.6) mm, 5 μm.

### Achiral Prep SFC

Instrument names: PIC-SFC-175; PIC-SFC-400
Method A: Mobile Phase A: CO₂: Co-solvent—0.5% Isopropylamine in MeOH (80:20); Flow Rate: 100 mL/min; % Co-Solvent: 20%; Column: 4-Ethyl pyridine (250×30) mm, 5 μm.
Method B: Mobile Phase A: CO₂: Co-solvent—0.5% Isopropylamine in MeOH (80:20); Flow Rate: 100 mL/min; % Co-Solvent: 20%; Column: DIOL (250×30) mm, 5 μm.
Method C: Mobile Phase A: CO₂: Co-solvent—0.5% Isopropylamine in MeOH (80:20); Flow Rate: 120 mL/min; % Co-Solvent: 20%; Column: DIOL (250×30) mm, 5 μm.
Method D: Mobile Phase A: CO₂: Co-solvent—0.5% Isopropylamine in MeOH (85:15), Flow Rate: 100 mL/min; % Co-Solvent: 15%; Column: DIOL (250×30) mm, 5 μm.
Method E: Mobile Phase A: CO₂: Co-solvent—0.5% Isopropylamine in MeOH (90:10); Flow Rate: 120 mL/min; % Co-Solvent: 10%; Column: 4-Ethyl pyridine (250×30) mm, 5 μm.
Method F: Mobile Phase A: CO₂: Co-solvent—0.5% Isopropylamine in MeOH (80:20); Flow Rate: 110 mL/min; % Co-Solvent: 20%; Column: DIOL (250×30) mm, 5 μm.
Method G: Mobile Phase A: CO₂: Co-solvent—0.5% Isopropylamine in IPA (75:25); Flow Rate: 120 mL/min; % Co-Solvent: 25%; Column: DIOL (250×30) mm, 5 μm.
Method H: Mobile Phase A: CO₂: Co-solvent—0.5% Isopropylamine in MeOH (85:15), Flow Rate: 110 mL/min; % Co-Solvent: 15%; Column: DIOL (250×30) mm, 5 μm.
Method I: Mobile Phase A: CO₂: Co-solvent—0.5% Isopropylamine in MeOH (90:10); Flow Rate: 120 mL/min; % Co-Solvent: 10%; Column: P4VP (250×30) mm, 5 μm.

### Intermediates

### Intermediate 1. tert-Butyl 3-(isopropylamino)azetidine-1-carboxylate

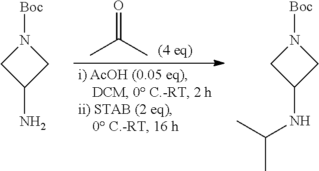

In a dried, 25 mL round bottom flask under a nitrogen atmosphere, tert-butyl 3-aminoazetidine-1-carboxylate (1 g, 5.81 mmol) was dissolved in DCM (20 mL). To this solution, acetone (1.349 g, 23.23 mmol) and AcOH (0.017 g, 0.290 mmol) were added at 0° C. and the reaction was stirred at RT for 2 h. To this reaction mixture, STAB (2.461 g, 11.61 mmol) was added at 0° C. and the reaction was stirred at RT for 16 h. The reaction progress was monitored by LCMS and TLC (5% MeOH in DCM). After completion, the reaction was quenched with saturated sodium bicarbonate solution (50 mL) and the mixture was extracted with DCM (3×50 mL). The combined organic layer was washed with ice-cold water (50 mL), dried over anhydrous sodium sulfate and filtered, and the filtrate was concentrated under reduced pressure to afford crude tert-butyl 3-(isopropylamino)azetidine-1-carboxylate (1.2 g, 96% yield) as a gummy liquid. This material was used without further purification. ¹H NMR (400 MHz, CDCl₃): δ 4.21-4.04 (m, 2H), 3.73-3.57 (m, 3H), 2.81 (spt, J=6.3 Hz, 1H), 1.42 (s, 9H), 1.03 (d, J=6.3 Hz, 6H), one proton merged with solvent peaks.

### Intermediate 2. tert-Butyl (R)-3-(isopropylamino)pyrrolidine-1-carboxylate

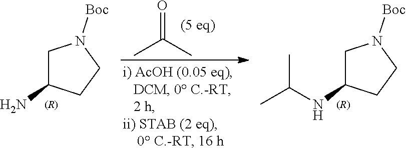

This compound was synthesized following the procedure described for the synthesis of Intermediate 1, starting with 1 equivalent of tert-butyl (R)-3-aminopyrrolidine-1-carboxylate, except that 5 equivalents of acetone was used. After completion, the reaction was quenched with saturated sodium bicarbonate solution and the mixture was extracted 10% MeOH DCM to obtain crude tert-butyl (R)-3-(isopropylamino)pyrrolidine-1-carboxylate as a liquid. This material was used without further purification.

Yield: 77%; ¹H NMR (400 MHz, DMSO-d₆): δ 3.44-3.35 (m, 1H), 3.34-3.27 (m, 2H), 3.25-3.09 (m, 1H), 2.96-2.82 (m, 1H), 2.81-2.67 (m, 1H), 1.97-1.92 (m, 1H), 1.65-1.44 (m, 1H), 1.39 (s, 9H), 0.99-0.93 (m, 6H), one proton merged with solvent peaks.

### Intermediate 3. tert-Butyl (S)-3-(isopropylamino)pyrrolidine-1-carboxylate

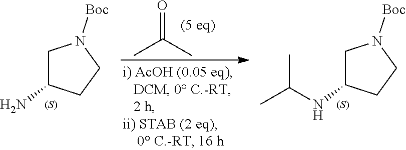

This compound was synthesized following the procedure described for the synthesis of Intermediate 1, starting with 1 equivalent of tert-butyl (S)-3-aminopyrrolidine-1-carboxylate, except that 5 equivalents of acetone was used. After completion, the reaction was quenched with saturated sodium bicarbonate solution and the mixture was extracted with 10% MeOH DCM to obtain crude tert-butyl (S)-3-(isopropylamino)pyrrolidine-1-carboxylate as a sticky liquid. This material was used without further purification.

Yield: 65.3%; ¹H NMR (400 MHz, DMSO-d₆): δ 3.42-3.25 (m, 3H), 3.24-3.10 (m, 1H), 2.93-2.81 (m, 1H), 2.81-2.67 (m, 1H), 2.12-1.85 (m, 1H), 1.69-1.45 (m, 1H), 1.39 (s, 9H), 0.99-0.94 (m, 6H), one proton merged with solvent peaks.

### Intermediate 4. tert-Butyl (3R,4R)-3-fluoro-4-(isopropylamino)pyrrolidine-1-carboxylate

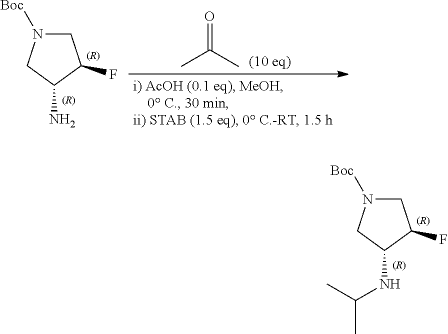

This compound was synthesized following the procedure described for the synthesis of Intermediate 1, starting with 1 equivalent of tert-butyl (3R,4R)-3-amino-4-fluoropyrrolidine-1-carboxylate, except that 10 equivalents of acetone, 0.1 equivalent of AcOH, and 1.5 equivalents of STAB were used, and MeOH was used as the solvent. After completion, the reaction was quenched with ice-cold water, aqueous Na₂CO₃ was added, and the mixture was extracted with EtOAc. The combined organic layer was washed with brine, dried over Na₂SO₄, and filtered, and the filtrate was concentrated under reduced pressure to afford crude tert-butyl (3R,4R)-3-fluoro-4-(isopropyl amino)pyrrolidine-1-carboxylate as a liquid. This material was used without further purification.

Yield: 82%; ¹H NMR (400 MHz, DMSO-d₆): δ 5.11-4.76 (m, 1H), 3.66-3.33 (m, 4H), 3.22-3.16 (m, 1H), 2.79 (spt, J=6.2 Hz, 1H), 2.01-1.70 (m, 1H), 1.41 (s, 9H), 1.03-0.94 (m, 6H).

### Intermediate 5. tert-Butyl 4-(isopropylamino)piperidine-1-carboxylate

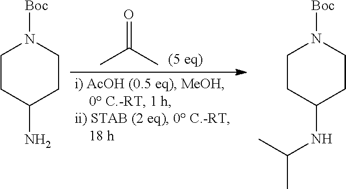

This compound was synthesized following the procedure described for the synthesis of Intermediate 1, starting with 1 equivalent of tert-butyl 4-aminopiperidine-1-carboxylate, except that 5 equivalents of acetone and 0.5 equivalent of AcOH were used, and MeOH was used as the solvent. After completion, the reaction was concentrated under reduced pressure and quenched with saturated sodium bicarbonate solution. The mixture was extracted with DCM to obtain crude tert-butyl 4-(isopropylamino)piperidine-1-carboxylate as a sticky liquid. This material was used without further purification.

Yield: 67.1%; ¹H NMR (400 MHz, CDCl₃): δ 4.07 (br s, 2H), 3.01 (spt, J=6.2 Hz, 1H), 2.87-2.65 (m, 3H), 1.92-1.79 (m, 2H), 1.50-1.44 (m, 9H), 1.32-1.13 (m, 2H), 1.08 (d, J=6.3 Hz, 6H), one proton merged with solvent peaks; LCMS (Method A): Rt=1.39 min, m/z=243.1 [M+H]⁺, 90.22%.

### Intermediate 6. 1-Cyclobutyl-N-isopropylazetidin-3-amine

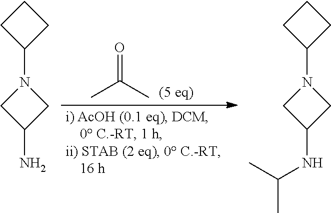

This compound was synthesized following the procedure described for the synthesis of Intermediate 1, starting with 1 equivalent of 1-cyclobutylazetidin-3-amine, except that 5 equivalents of acetone and 0.1 equivalent of AcOH were used to obtain crude 1-cyclobutyl-N-isopropylazetidin-3-amine as a liquid. This material was used without further purification.

Yield: 52.1%; ¹H NMR (400 MHz, DMSO-d₆): δ 3.38-3.31 (m, 2H), 3.26 (quin, J=6.5 Hz, 1H), 3.04-2.95 (m, 1H), 2.67 (spt, J=6.3 Hz, 1H), 2.61-2.56 (m, 2H), 1.91-1.81 (m, 2H), 1.76-1.53 (m, 4H), 0.92 (d, J=6.3 Hz, 6H); LCMS (Method A): Rt=1.26 min, m/z=169.1 [M+H]⁺, 63.11%.

### Intermediate 7. tert-Butyl ((1r,3r)-3-(isopropylamino)cyclobutyl)carbamate

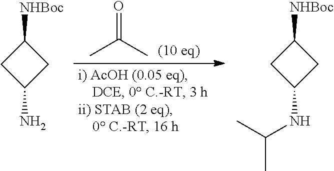

This compound was synthesized following the procedure described for the synthesis of Intermediate 1, starting with 1 equivalent of tert-butyl ((1r,3r)-3-aminocyclobutyl)carbamate, except that 10 equivalents of acetone was used, and DCE was used as the solvent. After work-up, the filtrate was concentrated under reduced pressure to obtain crude tert-butyl ((1r,3r)-3-(isopropylamino)cyclobutyl)carbamate as a semi-solid. This material was used without further purification.

Yield: 82%; ¹H NMR (400 MHz, CDCl₃): δ 4.79 (br s, 1H), 4.17-4.02 (m, 1H), 3.53-3.47 (m, 1H), 2.84-2.73 (m, 1H), 2.17-2.08 (m, 4H), 1.44 (s, 9H), 1.02 (d, J=6.3 Hz, 6H), one proton merged with solvent peaks.

### Intermediate 8. tert-Butyl ((1r,3r)-3-(isopropylamino)-1-methylcyclobutyl)carbamate

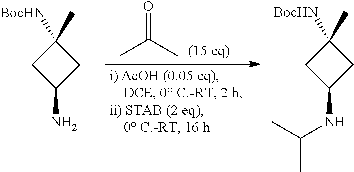

This compound was synthesized following the procedure described for the synthesis of Intermediate 1, starting with 1 equivalent of tert-butyl ((1r,3r)-3-amino-1-methylcyclobutyl)carbamate, except that 15 equivalents of acetone was used, and DCE was used as the solvent. After work-up, the filtrate was concentrated under reduced pressure to obtain crude tert-butyl ((1r,3r)-3-(isopropylamino)-1-methylcyclobutyl)carbamate as a semi-solid. This material was used without further purification.

Yield: 99%; ¹H NMR (400 MHz, DMSO-d₆): δ 6.82 (br s, 1H), 3.18 (quin, J=7.6 Hz, 1H), 2.71-2.59 (m, 1H), 2.47-2.34 (m, 2H), 1.55-1.48 (m, 2H), 1.38 (s, 9H), 1.28 (s, 3H), 0.91 (d, J=6.2 Hz, 6H), one proton merged with solvent peaks.

### Intermediate 9. tert-Butyl ((1R,3S)-3-(isopropylamino)cyclopentyl)carbamate

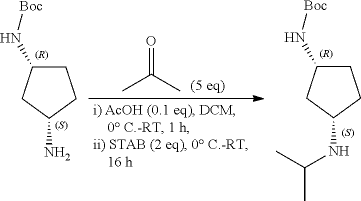

This compound was synthesized following the procedure described for the synthesis of Intermediate 1, starting with 1 equivalent of tert-butyl ((1R,3S)-3-aminocyclopentyl)carbamate, except that 5 equivalents of acetone and 0.1 equivalent of AcOH were used to obtain crude tert-butyl ((1R,3S)-3-(isopropylamino)cyclopentyl)carbamate as a sticky liquid. This material was used without further purification.

Yield: 98%; Yield: 98%; ¹H NMR (400 MHz, DMSO-d₆): δ 6.85 (br d, J=7.0 Hz, 1H), 3.89-3.63 (m, 1H), 3.23-3.07 (m, 1H), 2.89-2.76 (m, 1H), 2.56-2.53 (m, 1H), 1.82-1.70 (m, 2H), 1.52-1.40 (m, 2H), 1.38 (s, 9H), 1.25-1.14 (m, 1H), 1.00 (d, J=6.3 hz, 6H), one proton merged with solvent peaks.

### Intermediate 10. 2-(2-Benzylamino)ethyl)pyridin-4-ol 2,2,2-trifluoroacetic Acid

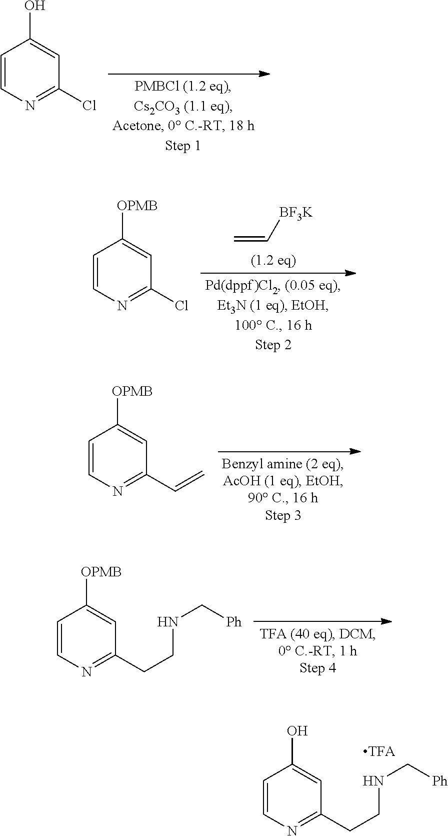

### Step 1. 2-Chloro-4-((4-methoxybenzyl)oxy)pyridine

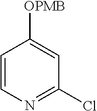

The reaction was performed following the same procedure in two batches (2×150 g).

In a 10 L three neck round bottom flask under nitrogen atmosphere, 2-chloropyridin-4-ol (150 g, 1158 mmol) was dissolved in acetone (2.5 L) at RT, and the reaction was cooled to 0° C. To this cooled solution, Cs₂CO₃ (415 g, 1274 mmol) was added, then 4-methoxybenzyl chloride (0.189 L, 1390 mmol) was added slowly using a dropping funnel. The reaction was stirred at RT for 18 h. The progress of the reaction was monitored by TLC (30% EtOAc in hexane). After completion, the two batches were combined and filtered, and the filter pad was washed with acetone (2×1 L). The filtrate was concentrated under reduced pressure, and the residue was diluted with water (1 L) and extracted with EtOAc (3×2.5 L). The combined organic layer was washed with brine solution (2×1 L), dried over anhydrous sodium sulfate, and filtered. The filtrate was concentrated under reduced pressure to obtain the crude compound. The crude was purified by silica gel flash column chromatography using EtOAc in hexane (product was eluted at 20/EtOAc in hexane) to obtain 2-chloro-4-((4-methoxybenzyl)oxy)pyridine (275 g, 44.3% yield, combined yield for 300 g batch) as a solid. ¹H NMR (400 MHz, DMSO-d₆): δ 8.21 (d, J=5.79 Hz, 1H), 7.40 (d, J=8.55 Hz, 2H), 7.18 (d, J=2.10 Hz, 1H), 7.06 (dd, J=5.79, 2.24 Hz, 1H), 6.97 (d, J=8.68 Hz, 2H), 5.15 (s, 2H), 3.76 (s, 3H); LCMS (Method A): Rt=1.96 min, m/z=250.0 [M+H]⁺, 93.16%.

### Step 2. 4-((4-Methoxybenzyl)oxy)-2-vinylpyridine

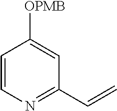

In a 10 L three neck round bottom flask, ethanol (3.5 L) was degassed by purging with argon gas for 15 min. To this solution, 2-chloro-4-((4-methoxybenzyl)oxy)pyridine (170 g, 681 mmol), potassium trifluoro(vinyl)borate (109 g, 817 mmol), TEA (96 mL, 681 mmol), and PdCl₂(dppf) (24.91 g, 34.0 mmol) were added at RT. The reaction was stirred at 100° C. for 16 h under an argon atmosphere. The progress of the reaction was monitored by TLC (50% EtOAc in hexane). After completion, the reaction was filtered through a Celite® pad, and the pad was washed with EtOAc (2×500 mL). The filtrate was concentrated under reduced pressure to obtain the crude compound. The crude was purified by flash column chromatography using EtOAc and hexane (product was eluted at 20% EtOAc in hexane) to obtain 4-((4-methoxybenzyl)oxy)-2-vinylpyridine (110 g, 63.4% yield) as a solid. ¹H NMR (400 MHz, DMSO-d₆): δ 8.35 (d, J=5.66 Hz, 1H), 7.41 (d, J=8.55 Hz, 2H), 7.14 (d, J=2.37 Hz, 1H), 6.96 (d, J=8.68 Hz, 2H), 6.92 (dd, J=2.50, 5.66 Hz, 1H), 6.75 (dd, J=10.72, 17.43 Hz, 1H), 6.24 (dd, J=1.64, 17.43 Hz, 1H), 5.45 (dd, J=1.64, 10.72 Hz, 1H), 5.13 (s, 2H), 3.77 (s, 3H); LCMS (Method A): Rt=1.97 min, m/z=242.2 [M+H]⁺, 94.67%.

### Step 3. N-Benzyl-2-(4-((4-methoxybenzyl)oxy)pyridin-2-yl)ethan-1-amine

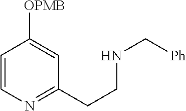

To a stirred solution of 4-((4-methoxybenzyl)oxy)-2-vinylpyridine (100 g, 414 mmol) in ethanol (2 L), benzylamine (91 mL, 829 mmol) and acetic acid (23.73 mL, 414 mmol) were added at RT under nitrogen atmosphere. The reaction was stirred at 90° C. for 16 h. The progress of the reaction was monitored by TLC (10% MeOH in DCM). After completion, the reaction was cooled to RT and concentrated under reduced pressure to obtain the crude compound. The crude was purified by silica gel flash column chromatography using MeOH in DCM (product was eluted at 10% MeOH in DCM) to afford N-benzyl-2-(4-((4-methoxybenzyl)oxy)pyridin-2-yl)ethan-1-amine (102 g, 56.1% yield) as a semi-solid. ¹H NMR (400 MHz, DMSO-d₆): δ 8.26 (d, J=5.75 Hz, 1H), 7.38 (d, J=8.76 Hz, 2H), 7.34-7.33 (m, 2H), 7.31-7.29 (m, 5H), 6.96-6.94 (m, 2H), 5.07 (s, 2H), 3.76 (s, 3H), 3.75 (s, 2H), 2.83-2.81 (m, 4H), one proton merged with solvent peaks; LCMS (Method D): Rt=1.96 min, m/z=349.0 [M+H]⁺, 79.48%.

### Step 4. 2-(2-(Benzylamino)ethyl)pyridin-4-ol 2,2,2-trifluoroacetic Acid

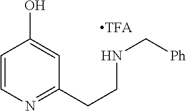 [SMILES: Oc1ccnc(CCNCc2ccccc2)c1]

To a stirred solution of N-benzyl-2-(4-((4-methoxybenzyl)oxy)pyridin-2-yl)ethan-1-amine (50 g, 143 mmol) in DCM (320 mL), TFA (439 mL, 5740 mmol) was added over 20 min at 0° C. The reaction was slowly warmed to RT and stirred for 1 h. The reaction turned purple from its initial pale-yellow color. The progress of the reaction was monitored by TLC (15% MeOH in DCM). After completion, the reaction was concentrated under reduced pressure to obtain the crude compound. The crude was purified by silica gel flash column chromatography using MeOH in DCM (product was eluted at 15% MeOH in DCM) to obtain 2-(2-(benzylamino)ethyl)pyridin-4-ol 2,2,2-trifluoroacetic acid (48.5 g, 75% yield) as a semi-solid. LCMS (Method A): Rt=1.15 min, m/z=229.1 [M+H]⁺, 76.16%.

### Intermediate 11. 6-Benzyl-5,6,7,8-tetrahydro-1,6-naphthyridin-4-ol

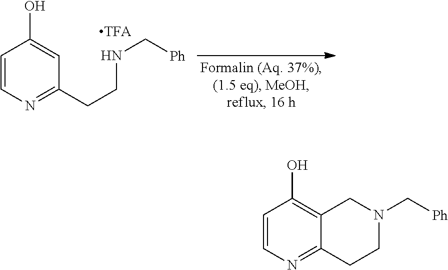 [SMILES: Oc1ccnc(CCNCc2ccccc2)c1 ; Oc1ccnc2c1CN(Cc1ccccc1)CC2]

To a stirred solution of 2-(2-(benzylamino)ethyl)pyridin-4-ol 2,2,2-trifluoroacetic acid (90 g, 263 mmol) in MeOH (2.5 L), formaldehyde solution (29.4 mL, 394 mmol; 37 wt % in water) was added slowly at RT. The reaction was stirred at 70° C. for 16 h. The progress of the reaction was monitored by TLC (10% MeOH in DCM). After completion, the reaction was concentrated under reduced pressure to obtain the crude compound. The crude was purified by silica gel flash column chromatography using MeOH in DCM (product was eluted at 15% MOH in DCM) to obtain 6-benzyl-5,6,7,8-tetrahydro-1,6-naphthyridin-4-ol (63 g, 99% yield) as semisolid. ¹H NMR (400 MHz, methanol-d₄) δ 11.91 (brs, 1H), 7.74 (d, J=7.2 Hz, 1H), 7.56-7.42 (m, 5H), 6.17 (d, J=6.8 Hz, 11H), 4.33 (br s, 2H), 3.78 (br s, 2H), 3.33 (br s, 2H), 2.99-2.90 (in, 2H); LCMS (Method A): Rt=1.26 min, m/z=241.1 [M+H]⁺, 98.99%.

### Intermediate 12. 6-Benzyl-5,6,7,8-tetrahydro-1,6-naphthyridin-4-ol Dihydrochloride

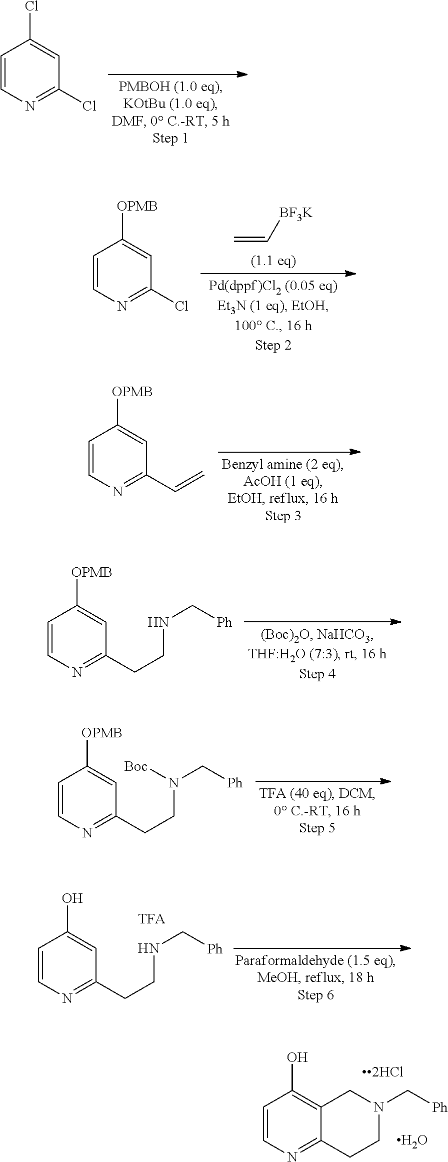

### Step 1. 2-Chloro-4-((4-methoxybenzyl)oxy)pyridine

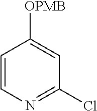

The reaction was performed following the same procedure in three batches (2 Kg, 10 Kg, and 12.1 Kg).

To a stirred solution of 4-methoxybenzyl alcohol (1.87 Kg, 0.0135 mol, 1.0 eq) in DMF (20.0 L) under a nitrogen atmosphere, KOtBu (1.52 Kg, 0.0135 mol, in 10 L DMF) was added at 0° C. over 1 h. To this reaction mixture, 2,4-dichloropyridine (2.0 Kg, 0.0135 mol) was added over 1 h at 0° C. using a dropping funnel. The reaction was stirred at 0-5° C. for 4-5 h. The reaction progress was monitored by TLC (30% EtOAc in hexane). After completion, water (40.0 L) was added to the reaction over 40-50 min at 0-5° C. using an addition funnel, and the reaction was maintained under stirring for 1-2 h at the same temperature. The resulting solid was filtered and washed with de-mineralized water (10.0 L). The wet solid was further subjected to slurry purification with pet ether (6.0 L). The solid was filtered and washed with pet ether (4.0 L). The resulting solid was further dried at 45-50° C. to obtain 2-chloro-4-((4-methoxybenzyl)oxy)pyridine (2.31 Kg, 68.4% yield) as a solid. All 3 batches were blended for pet ether slurry purification.

Combined yield: 27.83 Kg, 68.4%; ¹H NMR (400 MHz, CDCl₃): δ 8.19 (d, J=5.6 Hz, 1H), 7.34 (d, J=8.4 Hz, 2H), 6.94 (d, J=2.0 Hz, 2H), 6.90 (d, J=2.0, Hz, 1H), 6.81 (dd, J=2.8, J=6.0 Hz, 1H), 5.02 (s, 2H), 3.82 (s, 3H); LCMS: RT=5.27 min, m/z=250.0 [M+H]⁺, 95.40%.

### Step 2. 4-((4-Methoxybenzyl)oxy)-2-vinylpyridine

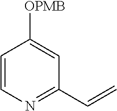

In a round bottom flask, 1,4-dioxane (20.0 L) was degassed with argon for 35-45 min. To this solvent, 2-chloro-4-((4-methoxybenzyl)oxy)pyridine (1.5 Kg, 6.007 mmol), potassium vinyltrifluoroborate (0.883 Kg, 6.607 mmol), and potassium carbonate (0.830 Kg, 6.007 mmol) were added at RT. The reaction mixture was again degassed with argon for 25-30 min at 25-35° C. To this reaction mixture, PdCl₂(dppf) (0.122 Kg, 0.15 mmol) was added and the reaction was heated at 90-95° C. for 3-4 h. The reaction progress was monitored by TLC (50% EtOAc in hexane). After completion, the reaction was cooled to 25-35° C. and diluted with EtOAc (7.5 L), and the mixture was filtered to remove insoluble inorganics. The bed was washed with EtOAc (6.0 L). The filtrate was distilled completely and re-dissolved in EtOAc (5.0 L). To this mixture, pet ether (10.0 L) was added and the mixture was stirred for 1-2 h at RT (Note: A brown-red colored gummy mass which does not contain any product adhered to the walls of the flask). The organic layer was filtered through a Celite® pad and the pad was washed with EtOAc:pet ether solution (4.5 L, 1.5:3, 3 times). The filtrate was concentrated under reduced pressure to afford crude 4-((4-methoxybenzyl)oxy)-2-vinylpyridine (1.08 Kg, 74% yield) as a gummy liquid. This material was used without further purification.

### Step 2 Scale Up Batches Summary Using the Above Procedure

| S. No. | 2-chloro-4-((4-methoxybenzyl)oxy)pyridine | | 4-((4-methoxybenzyl)oxy)-2-vinylpyridine |
|---|---|---|---|
| Batch-1 | 2 × 1.5 | Kg | lot-1: 18.0 Kg |
| Batch-2 | 2.0 | Kg | % yield: crude weight |
| Batch-3 | 2 × 3.0 | Kg | LCMS Purity: 95.61% |
| Batch-4 | 2 × 3.0 | Kg | Lot-2 (i.e., 2^{nd} crop): Filtrate MLR was |
| | | | distilled and subjected to re-purification |
| Batch-5 | 2 × 3.0 | Kg | in EtOAc/pet ether to obtain crude in |
| Batch-6 | 2.0 | Kg | 4.40 Kg with 83.67% HPLC purity |

Note: All above batches were combined for single distillation on Crop-1 to obtain product 18.0 Kg. 2^{nd} Crop: Filtrated MLR was distilled and subjected to re-purification to obtain 4.40 Kg, HPLC purity: 83.67%. Total starting material used: 25 Kg, product obtained: 22.4 Kg.

Combined yield: 22.4 Kg, 86.3%; ¹H NMR (400 MHz, CDCl₃): δ 8.40 (d, J=5.6 Hz, 1H), 7.35 (d, J=8.4 Hz, 2H), 6.94-6.91 (m, 3H), 6.79-6.72 (m, 2H), 6.19 (dd, J=1.2, J=17.2 Hz, 1H), 5.46 (dd, J=0.8 Hz, 10.8 Hz, 1H), 5.03 (s, 2H), 3.82 (s, 3H); HPLC Purity: 95.61%; MS: m/z=242.2 [M+H]⁺.

### Step 3. N-Benzyl-2-(4-((4-methoxybenzyl)oxy)pyridin-2-yl)ethan-1-amine

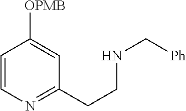

To a stirred solution of 4-((4-methoxybenzyl)oxy)-2-vinylpyridine (17.0 Kg, 70.455 mmol) in ethanol (340 L) under a nitrogen atmosphere, benzylamine (22.65 Kg, 211.36 mmol) and acetic acid (4.22 Kg, 70.455 mmol) were added at RT. The reaction was stirred at 90-95° C. for 16 h. The reaction progress was monitored by TLC (10% MeOH in DCM). After completion, the reaction was cooled to RT and distilled under reduced pressure at 45-50° C. to obtain crude N-benzyl-2-(4-((4-methoxybenzyl)oxy)pyridin-2-yl)ethan-1-amine (33.0 Kg, 75.3% yield) as a gummy liquid. HPLC purity: 56.17%. This material was used without further purification.

### Step 4. tert-Butyl benzyl(2-(4-((4-methoxybenzyl)oxy)pyridin-2-yl)ethyl)carbamate

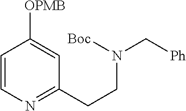

To a stirred solution of crude N-benzyl-2-(4-((4-methoxybenzyl)oxy)pyridin-2-yl)ethan-1-amine (54.0 g, 154.97 mmol) in THF (378 mL), aqueous sodium bicarbonate solution (52.06 g, in 162 mL of water) was added at RT. The reaction mass was cooled to 10-15° C. and (Boc)₂O (67.64 g, 309.94 mmol) was added using an addition funnel for 1-2 h. The reaction was warmed to RT and allowed to stir for 18-20 h. The reaction was monitored by HPLC. After completion, the reaction was quenched with water (216 mL), and the mixture was extracted with EtOAc (2×243 mL) at 25-35° C. The combined organic layer was dried over anhydrous sodium sulfate and filtered. The filtrate was distilled under reduced pressure at 40-45° C. and the crude was purified by silica gel column chromatography using 15% EtOAc in heptane to obtain tert-butyl benzyl(2-(4-((4-methoxybenzyl)oxy)pyridin-2-yl)ethyl)carbamate (41.5 g, 50.0% yield for 2 steps). ¹H NMR (400 MHz, CDCl₃): δ 8.32 (s, 1H), 7.34-7.28 (m, 4H), 7.25-7.21 (m, 3H), 6.93 (d, J=8.8 Hz, 2H), 6.79-6.63 (m, 2H), 4.99 (s, 2H), 4.38 (d, 1=11.2 Hz, 2H), 3.82 (s, 3H), 3.59-3.49 (m, 2H), 2.98-2.90 (m, 2H), 1.44 (s, 9H); HPLC Purity: 98.11%; LCMS: RT=6.15 min, m/z=449 [M+H]⁺.

### Step 5. 2-(2-(Benzylamino)ethyl)pyridin-4-ol 2,2,2-trifluoroacetic Acid

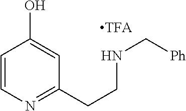 [SMILES: Oc1ccnc(CCNCc2ccccc2)c1]

To a stirred solution of tert-butyl benzyl(2-(4-((4-methoxybenzyl)oxy)pyridin-2-yl)ethyl)carbamate (500 g, 1114.7 mmol) in DCM (5.0 L) under a nitrogen atmosphere, TFA (4.0 L) was added over 45-50 min at 0° C. The reaction was stirred RT for 16 h. The reaction mixture turned purple from the initial pale-yellow color. The reaction progress was monitored by TLC (10% MeOH in DCM). After completion, the reaction was concentrated under reduced pressure to obtain crude 2-(2-(benzylamino)ethyl)pyridin-4-ol 2,2,2-trifluoroacetic acid (1.35 Kg, crude HPLC purity: 77.06%) as a semisolid. This material was used without further purification.

### Step 6. 6-Benzyl-5,6,7,8-tetrahydro-1,6-naphthyridin-4-ol Dihydrochloride Hydrate

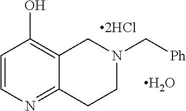

To a stirred solution of crude 2-(2-(benzylamino)ethyl)pyridin-4-ol 2,2,2-trifluoroacetic acid (1.35 Kg, 204 mmol) in methanol (8.1 L) under a nitrogen atmosphere, paraformaldehyde (22.84 mL, 307 mmol) was at RT. The reaction mixture was stirred at reflux temperature for 18 h. The reaction progress was monitored by HPLC (HPLC purity: product: 93.08%, SM: 1.91%). After completion, the reaction was concentrated under reduced pressure at 50° C. to obtain the crude compound. The crude was diluted with EtOAc (1.50 L). To this mixture, 4.0 M HCl (1.50 L, in EtOAc) was added at 0-5° C. and the reaction was stirred at 25-35° C. for 5-6 h. The reaction mixture was filtered and the wet cake was washed with EtOAc (1 L). The resulting solid was dried by sucking air through the filter cake for 1 hr. The resulting solid was further purified by 5% MeOH in DCM (1.0 L) slurry at RT. The solid was filtered and washed with DCM (1.0 L). The solid material was dried at 45-50° C. for 8-9 h to obtain 6-benzyl-5,6,7,8-tetrahydro-1,6-naphthyridin-4-ol dihydrochloride hydrate (250 g, 75.4% yield for 2 steps) as a solid. ¹H NMR (400 MHz, D₂O): δ 8.24 (d, J=6.8 Hz, 1H), 7.58-7.53 (m, 5H), 7.02 (d, J=7.2 Hz, 1H), 4.59 (s, 2H), 4.33-4.31 (m, 2H), 3.86-3.64 (m, 2H), 3.38 (t, J=5.6 Hz, 2H); LCMS: RT=2.94 min, m/z=241.2 [M+H]⁺; HPLC purity: 98.83%.

### Intermediate 13. tert-Butyl 6-iodo-2-azaspiro[3.3]heptane-2-carboxylate

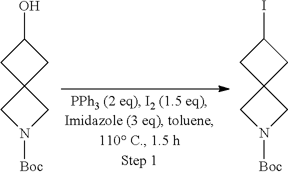 [SMILES: CC(C)(C)OC(=O)N1CC2(CC(O)C2)C1 ; CC(C)(C)OC(=O)N1CC2(CC(I)C2)C1]

To a solution of tert-butyl 6-hydroxy-2-azaspiro[3.3]heptane-2-carboxylate (tert-butyl 6-hydroxy-2-azaspiro[3.3]heptane-2-carboxylate (25 g, 117 mmol) in toluene (250 mL), imidazole (23.94 g, 352 mmol), triphenylphosphine (61.5 g, 234 mmol) and iodine (44.6 g, 176 mmol)) were added at RT. The reaction was stirred at 110° C. for 1.5 h. The progress of the reaction was monitored by TLC (30% EtOAc in hexane). After completion, the reaction was cooled to RT, and the solvent was evaporated under reduced pressure. To this residue, water (500 mL) was added, and the mixture was extracted with EtOAc (3×200 mL). The combined organic layer was washed with brine solution (100 mL), dried over anhydrous sodium sulfate, and filtered, and the filtrate was concentrated under reduced pressure to obtain the crude compound. The crude was purified by silica gel flash column chromatography using EtOAc in hexane (product was eluted at 20-25% EtOAc in hexane) to obtain tert-butyl 6-iodo-2-azaspiro[3.3]heptane-2-carboxylate (25 g, 63.4% yield) as a solid. ¹H NMR (400 MHz, DMSO-d₆): δ 4.43 (p, J=7.8 Hz, 1H), 3.85 (br s, 4H), 2.95-2.83 (m, 2H), 2.70-2.60 (m, 2H), 1.36 (s, 9H); LCMS (Method A): Rt=2.11 min, m/z: 268.1 [M−56]⁺, 96.11%.

### Intermediate 14. Benzyl 6-iodo-2-azaspiro[3.3]heptane-2-carboxylate

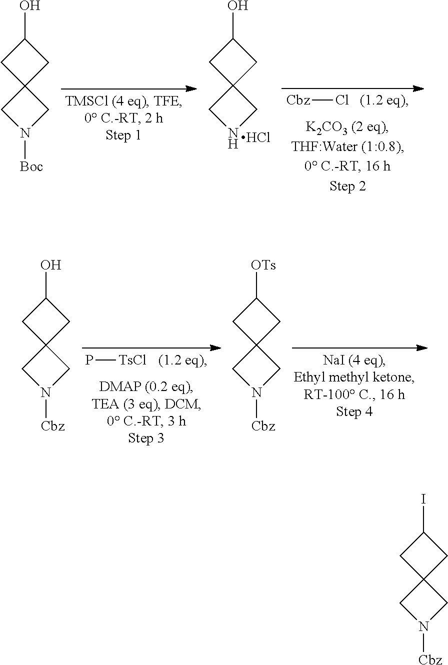

### Step 1. 2-Azaspiro[3.3]heptan-6-ol Hydrochloride

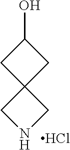

In a 3 L three neck round bottom flask under a nitrogen atmosphere, tert-butyl 6-hydroxy-2-azaspiro[3.3]heptane-2-carboxylate (200 g, 938 mmol) was dissolved in 2,2,2-trifluoroethanol (2.5 L). The reaction was cooled to 0° C., and TMSCl (0.476 L, 3751 mmol) was added slowly through a dropping funnel. The reaction was stirred at RT for 2 h. The progress of the reaction was monitored by TLC (100% EtOAc). After completion, the reaction was concentrated under reduced pressure to obtain the crude compound. The crude was triturated with ethyl acetate (2×500 mL) and dried under reduced pressure to obtain 2-azaspiro[3.3]heptan-6-ol hydrochloride (130 g, 92% yield) as a solid. ¹H NMR (400 MHz, DMSO-d₆): δ 9.15 (br s, 2H), 5.11 (d, J=6.00 Hz, 1H), 3.99-3.90 (m, 1H), 3.88 (s, 2H), 3.84 (s, 2H), 2.49-2.43 (m, 2H), 2.01-1.91 (m, 2H); LCMS (Method A): Rt=0.41 min, m/z=114.1 [M+H]⁺, 99.40%. This material was used without further purification.

### Step 2. Benzyl 6-hydroxy-2-azaspiro[3.3]heptane-2-carboxylate

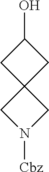 [SMILES: O=C(OCc1ccccc1)N1CC2(CC(O)C2)C1]

In a 3 L three neck round bottom flask under a nitrogen atmosphere, 2-azaspiro[3.3]heptan-6-ol hydrochloride (130 g, 869 mmol) was dissolved in THF (1 L) and water (800 mL) at RT. The reaction was cooled to 0° C., and K₂CO₃ (240 g, 1738 mmol) was added. To this reaction mixture, benzyl chloroformate (0.147 L, 1043 mmol) was added slowly using a dropping funnel. The reaction was stirred at RT for 16 h. The progress of the reaction was monitored by TLC (100% EtOAc). After completion, the reaction was diluted with water (1.5 L) and extracted with EtOAc (2×2.5 L). The combined organic layer was washed with brine solution (1 L), dried over anhydrous sodium sulfate, and filtered, and the filtrate was concentrated under reduced pressure to obtain the crude compound. The crude was purified by silica gel flash column chromatography using EtOAc in hexane (the product was eluted at 80% EtOAc in hexane) to obtain benzyl 6-hydroxy-2-azaspiro[3.3]heptane-2-carboxylate (210 g, 93% yield) as a solid. ¹H NMR (400 MHz, DMSO-d₆): δ 7.40-7.28 (m, 5H), 5.03-4.99 (m, 3H), 3.98-3.79 (m, 5H), 2.45-2.36 (m, 2H), 1.98-1.89 (m, 2H); LCMS (Method A): Rt=1.47 min, m/z=248.2 [M+H]⁺, 95.12%.

### Step-3. Benzyl 6-(tosyloxy)-2-azaspiro[3.3]heptane-2-carboxylate

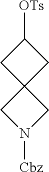 [SMILES: Cc1ccc(S(=O)(=O)OC2CC3(C2)CN(C(=O)OCc2ccccc2)C3)cc1]

In a 5 L three neck round bottom flask under a nitrogen atmosphere, benzyl 6-hydroxy-2-azaspiro[3.3]heptane-2-carboxylate (210 g, 849 mmol) was dissolved in DCM (2 L). The reaction was cooled to 0° C., and TEA (0.355 L, 2548 mmol) and DMAP (20.75 g, 170 mmol) were added. To this reaction mixture, p-toluenesulfonyl chloride (194 g, 1019 mmol) was added portion-wise at the same temperature. The resulting reaction was stirred at RT for 3 h. The progress of the reaction was monitored by TLC (100% EtOAc). After completion, the reaction was diluted with water (1 L) and extracted with DCM (2×2 L). The combined organic layer was dried over anhydrous sodium sulfate and filtered, and the filtrate was concentrated under reduced pressure to obtain the crude compound. The crude was purified by silica gel flash column chromatography using EtOAc in hexane followed by DCM (product was eluted at 100% DCM) to obtain benzyl 6-(tosyloxy)-2-azaspiro[3.3]heptane-2-carboxylate (270 g, 79% yield) as a solid. ¹H NMR (400 MHz, DMSO-d₆): δ 7.77 (d, J=8.00 Hz, 2H), 7.48 (d, J=8.00 Hz, 2H), 7.39-7.28 (m, 5H), 4.99 (s, 2H), 4.79-4.67 (m, 1H), 3.97-3.78 (m, 4H), 2.48-2.44 (m, 2H), 2.43 (s, 31H), 2.27-2.20 (m, 2H); LCMS (Method B): Rt=2.66 min, m/z=402.2 [M+H]⁺, 99.59%.

### Step-4. Benzyl 6-iodo-2-azaspiro[3.3]heptane-2-carboxylate

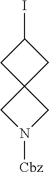 [SMILES: O=C(OCc1ccccc1)N1CC2(CC(I)C2)C1]

In a 3 L three neck round-bottom flask under a nitrogen atmosphere, benzyl 6-(tosyloxy)-2-azaspiro[3.3]heptane-2-carboxylate (270 g, 673 mmol) was dissolved in ethyl methyl ketone (2 L). To this solution, sodium iodide (403 g, 2690 mmol) was added portion-wise at RT, and the reaction was heated to 100° C. for 16 h. The progress of the reaction was monitored by TLC (100% EtOAc). After completion, the reaction was cooled to RT, diluted with water (2 L), and extracted with EtOAc (3×1.5 L). The combined organic layer was washed with brine solution (1 L), dried over anhydrous sodium sulfate, and filtered, and the filtrate was concentrated under reduced pressure to obtain the crude compound. The crude was purified by silica gel flash column chromatography using MeOH in DCM (product was eluted at 5% MeOH in DCM) to obtain benzyl 6-iodo-2-azaspiro[3.3]heptane-2-carboxylate (205 g, 81% yield) as a solid. ¹H NMR (400 MHz, DMSO-d₆): δ 7.40-7.29 (m, 5H), 5.01 (s, 2H), 4.43 (p, J=8 Hz, 1H), 4.03-3.89 (m, 4H), 2.96-2.87 (m, 2H), 2.72-2.62 (m, 2H); LCMS (Method D): Rt=2.54 min, m/z=358.1 [M+H]⁺, 94.78%.

### Intermediate 15. tert-Butyl 4-((2-azaspiro[3.3]heptan-6-yl)oxy)-7,8-dihydro-1,6-naphthyridine-6(5H)-carboxylate

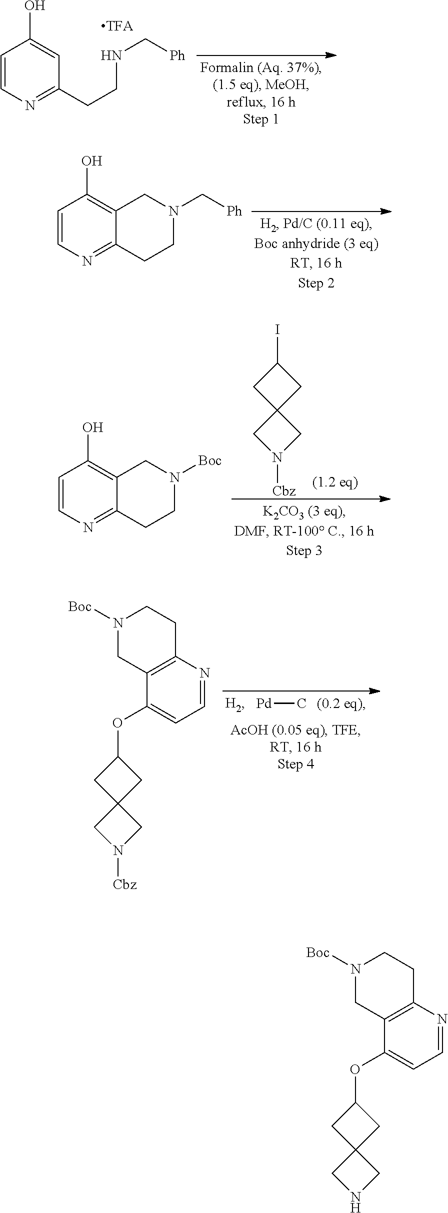

### Step 1. 6-Benzyl-5,6,7,8-tetrahydro-1,6-naphthyridin-4-ol

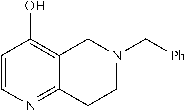 [SMILES: Oc1ccnc2c1CN(Cc1ccccc1)CC2]

To a stirred solution of 2-(2-(benzylamino)ethyl)pyridin-4-ol 2,2,2-trifluoroacetic acid (90 g, 263 mmol) in MeOH (2.5 L), formaldehyde solution (29.4 mL, 394 mmol; 37 wt % in water) was added slowly at RT. The reaction was stirred at 70° C. for 16 h. The progress of the reaction was monitored by TLC (10% MeOH in DCM). After completion, the reaction was concentrated under reduced pressure to obtain the crude compound. The crude was purified by silica gel flash column chromatography using MeOH in DCM (product was eluted at 15% MeOH in DCM) to obtain 6-benzyl-5,6,7,8-tetrahydro-1,6-naphthyridin-4-ol (63 g, 99% yield) as semisolid. ¹H NMR (400 MHz, methanol-d₄) δ 11.91 (brs, 1H), 7.74 (d, J=7.2 Hz, 1H), 7.56-7.42 (m, 5H), 6.17 (d, J=6.8 Hz, 1H), 4.33 (br s, 2H), 3.78 (br s, 2H), 3.33 (br s, 2H), 2.99-2.90 (in, 2H); LCMS (Method A): Rt=1.26 min, m/z=241.1 [M+H]⁺, 98.99%.

### Step 2. tert-Butyl 4-hydroxy-7,8-dihydro-1,6-naphthyridine-6(5H)-carboxylate

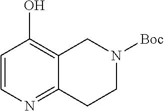 [SMILES: CC(C)(C)OC(=O)N1CCc2nccc(O)c2C1]

In a 2 L three neck round bottom flask under an argon atmosphere, 6-benzyl-5,6,7,8-tetrahydro-1,6-naphthyridin-4-ol (35 g, 146 mmol) was dissolved in MeOH (350 mL). To this solution, Pd—C (17.5 g, 16.44 mmol) and (Boc)₂O (101 mL, 437 mmol) were added, and the reaction was stirred at RT for 16 h under a hydrogen atmosphere (balloon pressure). The progress of the reaction was monitored by TLC (10% MeOH in DCM). After completion, the reaction was filtered through a Celite® pad, and the pad was washed with MeOH (2×100 mL). The filtrate was concentrated under reduced pressure to obtain the crude compound. The crude was purified by silica gel flash column chromatography using MeOH in DCM (product eluted at 10% MeOH in DCM) to obtain tert-butyl 4-hydroxy-7,8-dihydro-1,6-naphthyridine-6(5H)-carboxylate (28 g, 74.8% yield) as fluffy solid. 1H NMR (400 MHz, DMSO-d₆): δ 7.99 (br s, 1H), 6.57 (br s, 1H), 4.25 (s, 2H), 3.61 (br t, J=5.32 Hz, 2H), 2.79 (br t, J=4.94 Hz, 2H), 1.43 (m, 9H), —OH proton not observed; LCMS (Method D): Rt=1.16 min, m/z=251.2 [M+H]⁺, 97.33%.

### Step 3. tert-Butyl 4-((2-((benzyloxy)carbonyl)-2-azaspiro[3.3]heptan-6-yl)oxy)-7,8-dihydro-1,6-naphthyridine-6(5H)-carboxylate

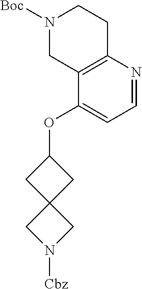 [SMILES: CC(C)(C)OC(=O)N1CCc2nccc(OC3CC4(C3)CN(C(=O)OCc3ccccc3)C4)c2C1]

To a stirred solution of tert-butyl 4-hydroxy-7,8-dihydro-1,6-naphthyridine-6(5H)-carboxylate (50 g, 200 mmol, 0.599 mmol) and benzyl 6-iodo-2-azaspiro[3.3]heptane-2-carboxylate (143 g, 400 mmol) in DMF (500 mL), K₂CO₃ (138 g, 999 mmol) was added at RT. The reaction was stirred at 100° C. for 16 h. The progress of the reaction was monitored by LCMS. After completion, the reaction was diluted with cold water (1 L) and extracted with EtOAc (2×1 L). The combined organic layer was washed with brine solution (1 L), dried over anhydrous sodium sulfate, and filtered. The filtrate was concentrated under reduced pressure to obtain the crude compound. The crude was purified by silica gel flash column chromatography using MeOH in DCM (the product was eluted in 10% MeOH in DCM) to obtain tert-butyl 4-((2-((benzyloxy)carbonyl)-2-azaspiro[3.3]heptan-6-yl)oxy)-7,8-dihydro-1,6-naphthyridine-6(5H)-carboxylate (90 g, 85% yield) as a solid. ¹H NMR (400 MHz, DMSO-d₆): δ 8.24 (d, J=5.6 Hz, 1H), 7.41-7.29 (m, 5H), 6.73 (d, J=5.7 Hz, 1H), 5.03 (s, 2H), 4.76 (quin, J=6.4 Hz, 1H), 4.36 (br s, 2H), 4.07-3.90 (m, 4H), 3.61 (br t, J=5.8 Hz, 2H), 2.82-2.75 (m, 4H), 2.30-2.20 (m, 2H), 1.42 (s, 9H). LCMS (Method G): Rt=2.18 min, m/z=480.3 [M+H]⁺, 93.85%.

### Step 4. tert-Butyl 4-((2-azaspiro[3.3]heptan-6-yl)oxy)-7,8-dihydro-1,6-naphthyridine-6(5H)-carboxylate

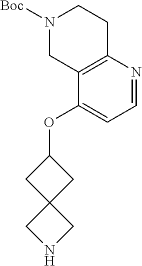 [SMILES: CC(C)(C)OC(=O)N1CCc2nccc(OC3CC4(CNC4)C3)c2C1]

In a 100 mL single neck round bottom flask under nitrogen atmosphere, tert-butyl 4-((2-((benzyloxy)carbonyl)-2-azaspiro[3.3]heptan-6-yl)oxy)-7,8-dihydro-1,6-naphthyridine-6(5H)-carboxylate (25 g, 52.1 mmol) was dissolved in 2,2,2-trifluoroethanol (250 mL). To this reaction mixture, Pd—C (24.96 g, 23.46 mmol) was added, and the reaction was stirred at RT for 16 h under a hydrogen atmosphere (balloon pressure). The progress of the reaction was monitored by TLC (100% EtOAc). After completion, the reaction was filtered through a Celite® pad, and the filtrate pad was washed with 10% MeOH in DCM (3×200 mL). The filtrate was concentrated under reduced pressure to obtain tert-butyl 4-((2-azaspiro[3.3]heptan-6-yl)oxy)-7,8-dihydro-1,6-naphthyridine-6(5H)-carboxylate (18 g, 94% yield) as a semi-solid. LCMS (Method G): Rt=1.35 min, m/z=346.1 [M+H]⁺, 93.78%.

### Intermediate 16. 4-((2-Azaspiro[3.3]heptan-6-yl)oxy)-6-benzyl-5,6,7,8-tetrahydro-1,6-naphthyridine Hydrochloride

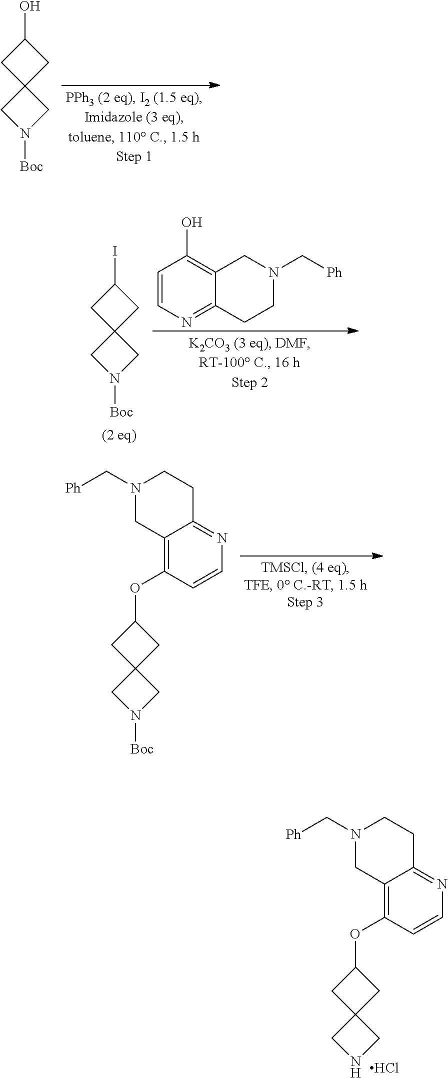

### Step 1. tert-Butyl 6-iodo-2-azaspiro[3.3]heptane-2-carboxylate

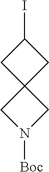 [SMILES: CC(C)(C)OC(=O)N1CC2(CC(I)C2)C1]

To a solution of tert-butyl 6-hydroxy-2-azaspiro[3.3]heptane-2-carboxylate (tert-butyl 6-hydroxy-2-azaspiro[3.3]heptane-2-carboxylate (25 g, 117 mmol) in toluene (250 mL), imidazole (23.94 g, 352 mmol), triphenylphosphine (61.5 g, 234 mmol) and iodine (44.6 g, 176 mmol)) were added at RT. The reaction was stirred at 110° C. for 1.5 h. The progress of the reaction was monitored by TLC (30% EtOAc in hexane). After completion, the reaction was cooled to RT, and the solvent was evaporated under reduced pressure. To this residue, water (500 mL) was added, and the mixture was extracted with EtOAc (3×200 mL). The combined organic layer was washed with brine solution (100 mL), dried over anhydrous sodium sulfate, and filtered, and the filtrate was concentrated under reduced pressure to obtain the crude compound. The crude was purified by silica gel flash column chromatography using EtOAc in hexane (product was eluted at 20-25% EtOAc in hexane) to obtain tert-butyl 6-iodo-2-azaspiro[3.3]heptane-2-carboxylate (25 g, 63.4% yield) as a solid. ¹H NMR (400 MHz, DMSO-d₆): δ 4.43 (p, J=7.8 Hz, 1H), 3.85 (br s, 4H), 2.95-2.83 (m, 2H), 2.70-2.60 (m, 2H), 1.36 (s, 9H); LCMS (Method A): Rt=2.11 min, m/z: 268.1 [M−56]⁺, 96.11%.

### Step 2. tert-Butyl 6-((6-benzyl-5,6,7,8-tetrahydro-1,6-naphthyridin-4-yl)oxy)-2-azaspiro[3.3]heptane-2-carboxylate

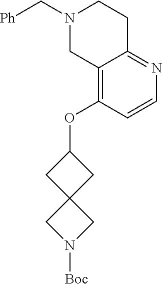 [SMILES: CC(C)(C)OC(=O)N1CC2(CC(Oc3ccnc4c3CN(Cc3ccccc3)CC4)C2)C1]

To a stirred solution of 6-benzyl-5,6,7,8-tetrahydro-1,6-naphthyridin-4-ol (104 g, 433 mmol) dissolved in DMF (1000 mL), powdered K₂CO₃ (179 g, 1298 mmol) was added at RT under a nitrogen atmosphere. The reaction was stirred at RT for 5 min. To this solution, tert-butyl 6-iodo-2-azaspiro[3.3]heptane-2-carboxylate (280 g, 866 mmol) was added at RT. The resulting mixture was stirred at RT for 10 min and then stirred at 100° C. for 16 h. The reaction was monitored by TLC (100% EtOAc). The reaction was quenched with water (3000 mL) and extracted with EtOAc (4500 mL). The organic layer was washed with cool brine (3×4500 mL), dried over anhydrous sodium sulfate, and filtered, and the filtrate was concentrated under reduced pressure. The crude material was purified by silica gel column chromatography using 100% EtOAc, and then with 10% MeOH in EtOAc, to afford tert-butyl 6-((6-benzyl-5,6,7,8-tetrahydro-1,6-naphthyridin-4-yl)oxy)-2-azaspiro[3.3]heptane-2-carboxylate (122 g, 64.4% yield) as a solid. ¹H NMR (400 MHz, DMSO-d₆): δ 8.20 (d, J=5.7 Hz, 1H), 7.38-7.32 (m, 4H), 7.31-7.23 (m, 1H), 6.65 (d, J=5.5 Hz, 1H), 4.68 (quin, J=6.7 Hz, 1H), 3.93-3.76 (m, 4H), 3.69 (s, 2H), 3.45 (s, 2H), 2.84-2.77 (m, 2H), 2.75-2.67 (m, 4H), 2.18-2.13 (m, 2H), 1.37 (s, 9H); LCMS (Method A): Rt=2.10 min, m/z=436.2 [M+H]⁺, 99.49%.

### Step 3. 4-((2-Azaspiro[3.3]heptan-6-yl)oxy)-6-benzyl-5,6,7,8-tetrahydro-1,6-naphthyridine Hydrochloride

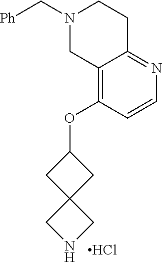 [SMILES: c1ccc(CN2CCc3nccc(OC4CC5(CNC5)C4)c3C2)cc1 ; Cl]

To a stirred solution of tert-butyl 6-((6-benzyl-5,6,7,8-tetrahydro-1,6-naphthyridin-4-yl)oxy)-2-azaspiro[3.3]heptane-2-carboxylate (70 g, 161 mmol) in 2,2,2-trifluoroethanol (700 mL), TMSCl (82 mL, 643 mmol) was added slowly at 0° C. The reaction was stirred at RT for 1.5 h. The progress of the reaction was monitored by LCMS. After completion, the reaction was concentrated under reduced pressure to obtain crude 4-((2-azaspiro[3.3]heptan-6-yl)oxy)-6-benzyl-5,6,7,8-tetrahydro-1,6-naphthyridine hydrochloride (60 g, 97% yield) as a solid. This material was used without further purification. LCMS (Method G): Rt=1.23 min, m/z=336.1 [M+H]⁺, 96.69%.

### Intermediate 17. 4-((2-Azaspiro[3.3]heptan-6-yl)oxy)-6-methyl-5,6,7,8-tetrahydro-1,6-naphthyridine Hydrochloride

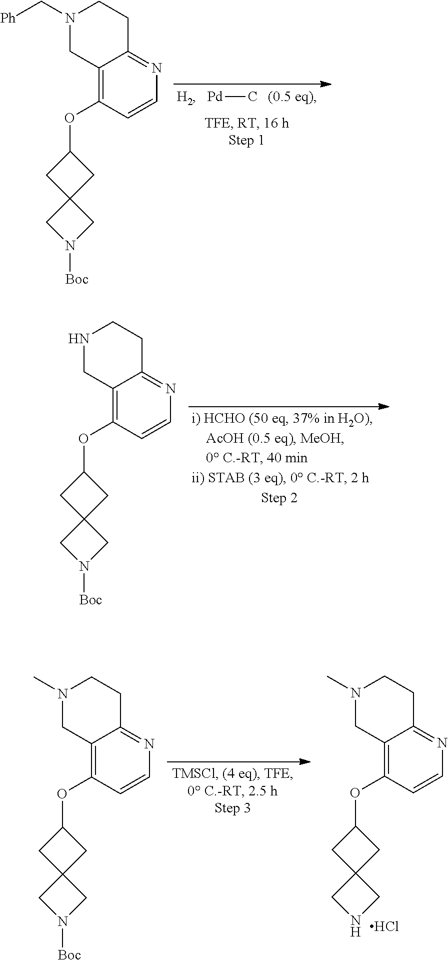

### Step 1. tert-Butyl 6-((5,6,7,8-tetrahydro-1,6-naphthyridin-4-yl)oxy)-2-azaspiro[3.3]heptane-2-carboxylate

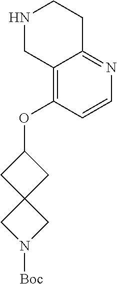 [SMILES: CC(C)(C)OC(=O)N1CC2(CC(Oc3ccnc4c3CNCC4)C2)C1]

The reaction was performed following the same procedure in two batches (20 g and 30 g).

In a dried, 500 mL two neck round bottom flask under a nitrogen atmosphere, tert-butyl 6-((6-benzyl-5,6,7,8-tetrahydro-1,6-naphthyridin-4-yl)oxy)-2-azaspiro[3.3]heptane-2-carboxylate (20 g, 45.9 mmol) was dissolved in TFE (300 mL). To this solution, Pd—C (24.43 g, 22.96 mmol) was added, and the reaction was stirred at RT for 16 h under a hydrogen atmosphere (balloon pressure). The reaction progress was monitored by LCMS and TLC (10% MeOH in DCM). After completion, the two batches were combined and filtered through a Celite® pad, and the pad was washed with 10% MeOH in DCM (3×150 mL). The filtrate was concentrated under reduced pressure to obtain crude tert-butyl 6-((5,6,7,8-tetrahydro-1,6-naphthyridin-4-yl)oxy)-2-azaspiro[3.3]heptane-2-carboxylate as a semi-solid (40 g, quantitative yield). This material was used without further purification. ¹H NMR (400 MHz, DMSO-d₆): δ 8.18 (d, J=5.5 Hz, 1H), 6.65 (d, J=5.6 Hz, 1H), 4.70 (quin, J=6.7 Hz, 1H), 3.93-3.88 (m, 4H), 3.71 (s, 2H), 2.98 (t, J=5.9 Hz, 2H), 2.75-2.67 (m, 4H), 2.24-2.16 (m, 2H), 1.37 (s, 9H), one proton merged with solvent peaks; LCMS (Method A): Rt=2.24 min, m/z=346.2 [M+H]⁺, 79.30%.

### Step 2. tert-Butyl 6-((6-methyl-5,6,7,8-tetrahydro-1,6-naphthyridin-4-yl)oxy)-2-azaspiro[3.3]heptane-2-carboxylate

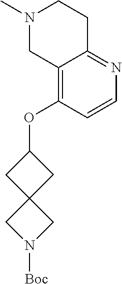 [SMILES: CN1CCc2nccc(OC3CC4(C3)CN(C(=O)OC(C)(C)C)C4)c2C1]

To a stirred solution of tert-butyl 6-((5,6,7,8-tetrahydro-1,6-naphthyridin-4-yl)oxy)-2-azaspiro[3.3]heptane-2-carboxylate (40 g, 116 mmol) in MeOH (400 mL), formaldehyde (431 mL, 5790 mmol, 37% in H₂O) and acetic acid (3.31 mL, 57.9 mmol) were added at 0° C. The reaction was stirred at RT for 40 min. To this reaction mixture, STAB (73.6 g, 347 mmol) was added at 0° C., and the reaction was stirred at RT for 2 h. The reaction progress was monitored by LCMS and TLC (10% MeOH in DCM). After completion, the reaction was filtered through a Celite® pad and the pad was washed with MeOH (2×100 mL). The crude was purified by silica gel flash column chromatography using MeOH in DCM (product was eluted at 10% MeOH in DCM) to obtain tert-butyl 6-((6-methyl-5,6,7,8-tetrahydro-1,6-naphthyridin-4-yl)oxy)-2-azaspiro[3.3]heptane-2-carboxylate (25 g, 59.7% yield). ¹H NMR (400 MHz, DMSO-d₆): δ 8.19 (d, J=5.6 Hz, 1H), 6.65 (d, J=5.6 Hz, 1H), 4.71 (quin, J=6.7 Hz, 1H), 3.91 (s, 2H), 3.83 (s, 2H), 3.36 (s, 2H), 2.84-2.79 (m, 2H), 2.76-2.71 (m, 2H), 2.66-2.61 (m, 2H), 2.37 (s, 3H), 2.25-2.18 (m, 2H), 1.37 (s, 9H). LCMS (Method A): Rt=1.77 min, m/z=360.1 [M+H]⁺, 98.83%; LCMS (Method A): Rt=1.78 min, m/z=360.1 [M+H]⁺, 98.83%.

### Step 3. 4-((2-Azaspiro[3.3]heptan-6-yl)oxy)-6-methyl-5,6,7,8-tetrahydro-1,6-naphthyridine Hydrochloride

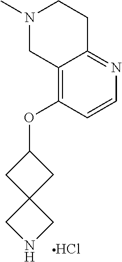 [SMILES: CN1CCc2nccc(OC3CC4(CNC4)C3)c2C1 ; Cl]

To a stirred solution of tert-butyl 6-((6-methyl-5,6,7,8-tetrahydro-1,6-naphthyridin-4-yl)oxy)-2-azaspiro[3.3]heptane-2-carboxylate (20 g, 55.6 mmol) in TFE (200 mL), TMSCl (28.4 mL, 223 mmol) was added at 0° C. The reaction was stirred at RT for 2.5 h. The reaction progress was monitored by LCMS. After completion, the reaction was concentrated under reduced pressure and the residue was triturated with EtOAc (2×100 mL). The solvent was decanted, and the solid was dried under vacuum to obtain crude 4-((2-azaspiro[3.3]heptan-6-yl)oxy)-6-methyl-5,6,7,8-tetrahydro-1,6-naphthyridine hydrochloride (20 g, quantitative yield). This material was used without further purification. LCMS (Method A): Rt=1.00 min, m/z=260.1 [M+H]⁺, 90.83%.

### Intermediate 18. (±)-1-((2-Azaspiro[3.3]heptan-6-yl)oxy)-5,6,8,9,10,10a-hexahydropyrrolo[2,1-f][1,6]naphthyridine

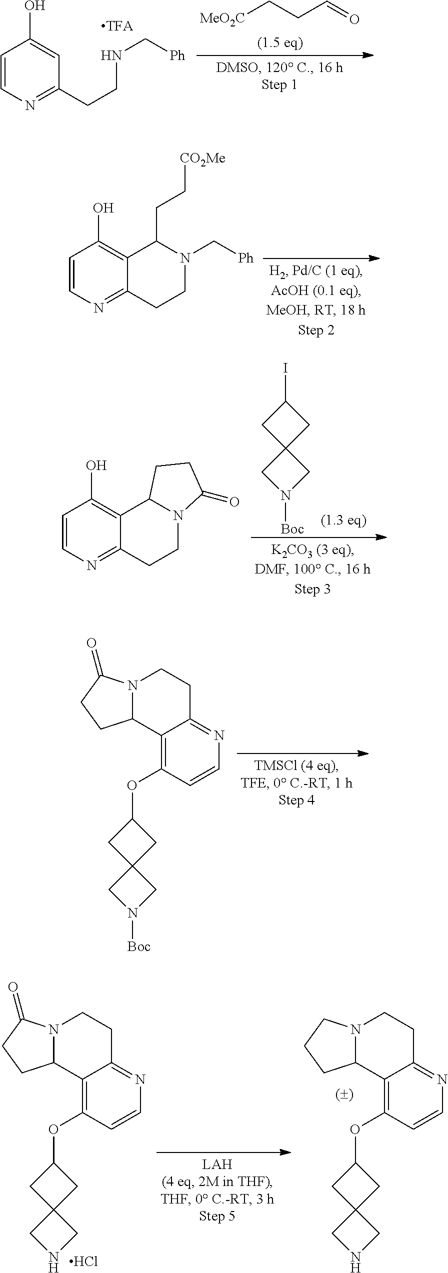

### Step 1. (±)-Methyl 3-(6-benzyl-4-hydroxy-5,6,7,8-tetrahydro-1,6-naphthyridin-5-yl)propanoate

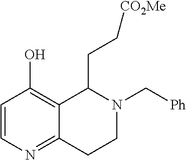 [SMILES: COC(=O)CCC1c2c(O)ccnc2CCN1Cc1ccccc1]

Step 1 was performed following the same procedure in four batches (4×500 mg).

In a 30 mL pressure vial under a nitrogen atmosphere, 2-(2-(benzylamino)ethyl)pyridin-4-ol 2,2,2-trifluoroacetic acid (500 mg, 1.461 mmol) was dissolved in DMSO (12.5 mL). To this solution, methyl 4-oxobutanoate (254 mg, 2.191 mmol) was added at RT. The reaction was stirred at 120° C. for 16 h. The reaction was monitored by TLC (10% MeOH in DCM). The crude reaction mixtures from four batches were combined for the workup. To this crude reaction mixture, ice-cold water (50 mL) was added, and the mixture was extracted with 10% MeOH in DCM (3×50 mL). The product was found in the aqueous layer. Therefore, the aqueous layer was basified with sodium bicarbonate solution (25 mL) and re-extracted with 10% MeOH in DCM (2×50 mL). The combined organic layer was washed with brine (50 mL), dried over anhydrous Na₂SO₄, and filtered, and the filtrate was concentrated under reduced pressure. The crude product was purified by silica gel flash column chromatography using MeOH in DCM (product eluted at 15% MeOH in DCM) to obtain (t)-methyl 3-(6-benzyl-4-hydroxy-5,6,7,8-tetrahydro-1,6-naphthyridin-5-yl)propanoate (837 mg, 43.88% combined yield) as a solid. LCMS (Method A): Rt=1.40 min, m/z=327.3 [M+H]⁺, 95.84%.

### Step 2. (±)-1-Hydroxy-5,9,10,10α-tetrahydropyrrolo[2,1-f][1,6]naphthyridin-8(6H)-one

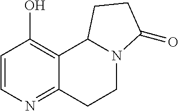 [SMILES: O=C1CCC2c3c(O)ccnc3CCN12]

In a 100 mL round bottom flask, (±)-methyl 3-(6-benzyl-4-hydroxy-5,6,7,8-tetrahydro-1,6-naphthyridin-5-yl)propanoate (1.2 g, 3.68 mmol) was dissolved in MeOH (23 mL) at RT. To this solution, AcOH (0.021 mL, 0.368 mmol) and Pd—C (3.91 g, 3.68 mmol) were added, and the reaction was stirred under a hydrogen atmosphere (balloon pressure) at RT for 18 h. The reaction progress was monitored by TLC (10% MeOH in DCM). The reaction was filtered through a Celite® pad, and the pad was washed with MeOH (2×50 mL). The filtrate was concentrated under reduced pressure. The crude compound was purified by reverse phase chromatography using a SiliCycle SiliaSep C18 column (40 g) on Biotage isolera one (Mobile phase A: 10 mM NH₄HCO₃ in water, B: ACN). The pure fractions were collected and concentrated under reduced pressure to afford (±)-1-hydroxy-5,9,10,10a-tetrahydropyrrolo[2,1-f][1,6]naphthyridin-8(6H)-one (480 mg, 63.91% yield) as a solid. ¹H NMR (400 MHz, DMSO-d₆): δ 11.38 (br s, 1H), 7.57 (d, J=7.3 Hz, 1H), 6.02 (d, J=7.0 Hz, 1H), 4.51 (t, J=7.8 Hz, 1H), 4.17-4.10 (m, 1H), 2.91-2.74 (m, 2H), 2.70-2.54 (m, 2H), 2.46-2.32 (m, 1H), 2.20-2.10 (m, 1H), 1.50-1.36 (m, 1H); LCMS (Method D): Rt=0.61 min, m/z=205.1 [M+H]⁺, 98.93%.

### Step 3. (®)-tert-Butyl 6-((8-oxo-5,6,8,9,10,10α-hexahydropyrrolo[2,1-f][1,6]naphthyridine-1-yl)oxy)-2-azaspiro[3.3]heptane-2-carboxylate

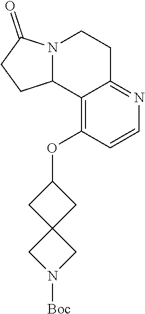 [SMILES: CC(C)(C)OC(=O)N1CC2(CC(Oc3ccnc4c3C3CCC(=O)N3CC4)C2)C1]

To a stirred solution of (±)-1-hydroxy-5,9,10,10a-tetrahydropyrrolo[2,1-f][1,6]naphthyridin-8(6H)-one (0.48 g, 2.350 mmol) in DMF (14.8 mL), K₂CO₃ (0.974 g, 7.05 mmol) was added at RT. The reaction was stirred at RT for 10 min. To this reaction mixture, tert-butyl 6-iodo-2-azaspiro[3.3]heptane-2-carboxylate (0.987 g, 3.06 mmol) was added, and the reaction was stirred at 100° C. for 16 h. The reaction was monitored by TLC (10% MeOH in DCM). After completion, the reaction was diluted with water (50 mL) and the mixture was extracted with DCM (2×50 mL). The combined organic layer was washed with ice-cold water (2×50 mL), dried over Na₂SO₄ and filtered, and the filtrate was concentrated under reduced pressure. The crude was purified by silica gel flash column chromatography using MeOH in DCM (product eluted at 8% MeOH in DCM) to obtain (f)-tert-butyl 6-((8-oxo-5,6,8,9,10,10a-hexahydropyrrolo[2,1-f][1,6]naphthyridin-1-yl)oxy)-2-azaspiro[3.3]heptane-2-carboxylate (0.55 g, 57.4% yield) as a solid. ¹H NMR (400 MHz, DMSO-d₆): δ 8.25 (d, J=5.5 Hz, 1H), 6.76 (d, J=5.7 Hz, 1H), 4.81-4.70 (m, 2H), 4.23-4.16 (m, 1H), 3.91 (s, 2H), 3.85 (s, 2H), 2.95-2.90 (m, 1H), 2.81-2.70 (m, 5H), 2.49-2.41 (m, 1H), 2.32-2.14 (m, 3H), 1.65-1.50 (m, 1H), 1.37 (s, 9H); LCMS (Method D): Rt=1.69 min, m/z=400.2 [M+H]⁺, 98.01%.

### Step 4. (=)-1-((2-Azaspiro[3.3]heptan-6-yl)oxy)-5,9,10,10α-tetrahydropyrrolo[2,1-f][1,6]naphthyridin-8(6H)-one Hydrochloride

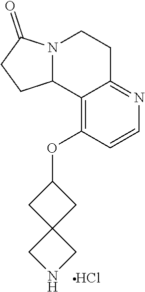 [SMILES: O=C1CCC2c3c(OC4CC5(CNC5)C4)ccnc3CCN12 ; Cl]

To a stirred solution of (±)-tert-butyl 6-((8-oxo-5,6,8,9,10,10a-hexahydropyrrolo[2,1-f][1,6]naphthyridin-1-yl)oxy)-2-azaspiro[3.3]heptane-2-carboxylate (0.2 g, 0.501 mmol) in 2,2,2-trifluoroethanol (10 mL), TMSCl (0.256 mL, 2.003 mmol) was added at 0° C. The reaction was stirred at RT for 1 h and the reaction progress was monitored by TLC (10% MeOH in DCM). After completion, the reaction was concentrated under reduced pressure. The crude compound was reconcentrated from EtOAc (50 mL), and the residue was dried under reduced pressure to obtain crude (±)-1-((2-azaspiro[3.3]heptan-6-yl)oxy)-5,9,10,10a-tetrahydropyrrolo[2,1-f][1,6]naphthyridin-8(6H)-one hydrochloride (0.16 g, 92% yield) as a solid. This material was used without further purification. ¹H NMR (400 MHz, DMSO-d₆): δ 8.92 (br s, 2H), 8.64 (br s, 1H), 7.28 (br s, 1H), 5.01 (br s, 1H), 4.80 (t, J=8.1 Hz, 1H), 4.32-4.19 (m, 1H), 4.03 (t, J=6.0 Hz, 2H), 3.98 (t, J=6.1 Hz, 2H), 3.06-2.72 (m, 6H), 2.47-2.34 (m, 3H), 2.30-2.22 (m, 1H), 1.75-1.63 (m, 1H); LCMS (Method A): Rt=0.99 min, m/z=300.3 [M+H]⁺, 97.41%. Step 5. (i)-1-((2-Azaspiro[3.3]heptan-6-yl)oxy)-5,6,8,9,10,10a-hexahydropyrrolo[2,1-f][1,6]naphthyridine

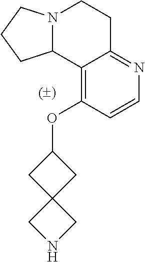 [SMILES: c1cc(OC2CC3(CNC3)C2)c2c(n1)CCN1CCCC21]

To a stirred solution of (i)-1-((2-azaspiro[3.3]heptan-6-yl)oxy)-5,9,10,10a-tetrahydropyrrolo[2,1-f][1,6]naphthyridin-8(6H)-one hydrochloride (1.6 g, 4.76 mmol) in dry THF (20 mL), lithium aluminum hydride (9.53 mL, 19.06 mmol, 2 M in THF) was added at 0° C. The reaction was stirred at RT for 4 h. The reaction progress was monitored by TLC (20% MeOH in DCM). After completion, the reaction was quenched with cold water (10 mL), and MeOH (30 mL) was added. The mixture was filtered through a sintered funnel using Whatman filter paper. The filtrate was concentrated under reduced pressure to afford crude (±)-1-((2-azaspiro[3.3]heptan-6-yl)oxy)-5,6,8,9,10,10a-hexahydropyrrolo[2,1-f][1,6]naphthyridine (1.5 g, quantitative yield) as a sticky liquid. This material was used without further purification. LCMS (Method B): Rt=0.21 min, m/z=286.4 [M+H]⁺, 99.85%.

### Intermediate 19. (±)-1-((2-Azaspiro[3.3]heptan-6-yl)oxy)-5,6,8,9,10,10a-hexahydropyrrolo[2,1-f][1,6]naphthyridine xHCl (Procedure 1)

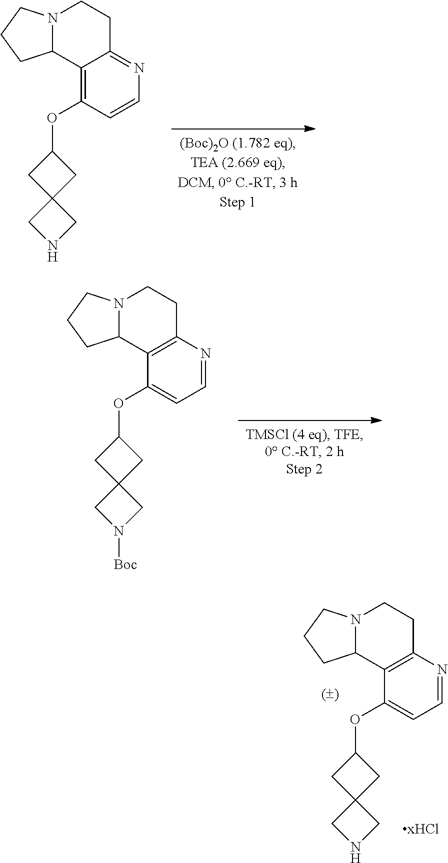 [SMILES: c1cc(OC2CC3(CNC3)C2)c2c(n1)CCN1CCCC21 ; CC(C)(C)OC(=O)N1CC2(CC(Oc3ccnc4c3C3CCCN3CC4)C2)C1 ; c1cc(OC2CC3(CNC3)C2)c2c(n1)CCN1CCCC21]

### Step 1. (±)-tert-Butyl 6-((5,6,8,9,10,10α-hexahydropyrrolo[2,1-f][1,6]naphthyridin-1-yl)oxy)-2-azaspiro[3.3]heptane-2-carboxylate

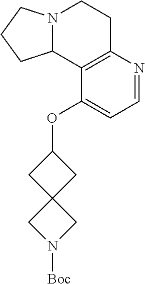 [SMILES: CC(C)(C)OC(=O)N1CC2(CC(Oc3ccnc4c3C3CCCN3CC4)C2)C1]

To a stirred solution of (±)-1-((2-azaspiro[3.3]heptan-6-yl)oxy)-5,6,8,9,10,10a-hexahydropyrrolo[2,1-f][1,6]naphthyridine (5 g, 17.46 mmol) in DCM (100 mL), TEA (6.51 mL, 46.6 mmol) and Boc-anhydride (7.14 mL, 31.1 mmol) were added at 0° C. under a nitrogen atmosphere. The reaction was stirred at RT for 3 h. The reaction was monitored by LCMS and TLC (5% MeOH in DCM). After completion, the reaction was quenched with water (200 mL), and the mixture was extracted with 10% MeOH in DCM (2×60 mL). The combined organic layer was washed with brine (50 mL), dried over anhydrous sodium sulfate, and filtered, and the filtrate was concentrated under reduced pressure. The crude product was purified by reverse phase chromatography using a SiliCycle SiliaSep C18 column (120 g) on Biotage isolera one (Mobile phase A: 10 mM NH₄HCO₃ in water, B: ACN), then by silica gel flash column chromatography using 0-100% EtOAc in hexane and then by 18% MeOH in DCM to obtain (t)-tert-butyl 6-((5,6,8,9,10,10a-hexahydropyrrolo[2,1-f][1,6]naphthyridin-1-yl)oxy)-2-azaspiro[3.3]heptane-2-carboxylate (1 g, 16.53% yield) as a solid. ¹H NMR (400 MHz, DMSO-d₆): δ 8.19 (d, J=5.6 Hz, 1H), 6.67 (d, J=5.6 Hz, 1H), 4.71 (quin, J=6.6 Hz, 1H), 3.98-3.78 (m, 4H), 3.74-3.66 (m, 1H), 3.05-2.83 (m, 3H), 2.80-2.65 (m, 5H), 2.47-2.38 (m, 1H), 2.26-2.15 (m, 2H), 1.87-1.70 (m, 2H), 1.56-1.44 (m, 1H), 1.37 (s, 9H); LCMS (Method C): Rt=1.57 min, m/z=386.3 [M+H]⁺, 99.72%.

### Step 2. (i)-1-((2-Azaspiro[3.3]heptan-6-yl)oxy)-5,6,8,9,10,10α-hexahydropyrrolo[2,1-f][1,6]naphthyridine xHCl

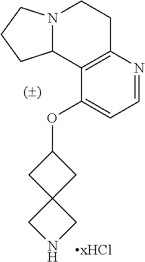 [SMILES: c1cc(OC2CC3(CNC3)C2)c2c(n1)CCN1CCCC21]

To a stirred solution of (f)-tert-butyl 6-((5,6,8,9,10,10a-hexahydropyrrolo[2,1-f][1,6]naphthyridin-1-yl)oxy)-2-azaspiro[3.3]heptane-2-carboxylate (0.95 g, 2.464 mmol) in TFE (10 mL), TMSCl (1.260 mL, 9.86 mmol) was added at 0° C. The reaction was stirred at RT for 2 h under a nitrogen atmosphere. The reaction progress was monitored by LCMS and TLC (10% MeOH in DCM). The reaction was concentrated under reduced pressure. The crude product was reconcentrated from EtOAc (20 mL) to obtain crude (±)-1-((2-azaspiro[3.3]heptan-6-yl)oxy)-5,6,8,9,10,10a-hexahydropyrrolo[2,1-f][1,6]naphthyridine xHCl (950 mg, quantitative yield) as a gummy solid. This material was used without further purification. ¹H NMR (400 MHz, DMSO-d₆): δ 11.98-11.78 (m, 1H), 9.05-8.85 (m, 2H), 8.67-8.52 (m, 1H), 7.23-7.06 (m, 1H), 5.01-4.89 (m, 1H), 4.75-4.62 (m, 1H), 4.06-3.95 (m, 4H), 3.64-3.54 (m, 2H), 3.28-3.19 (m, 2H), 2.94-2.82 (m, 2H), 2.72-2.62 (m, 1H), 2.44-2.35 (m, 2H), 2.13-2.02 (m, 2H), 1.90-1.81 (m, 1H), two protons merged with solvent peaks; LCMS (Method B): Rt=0.189 min, m/z=286.4 [M+H]⁺, 95.87%.

### Intermediate 19. (±)-1-((2-Azaspiro[3.3]heptan-6-yl)oxy)-5,6,8,9,10,10a-hexahydropyrrolo[2,1-f][1,6]naphthyridine xHCl (Procedure 2)

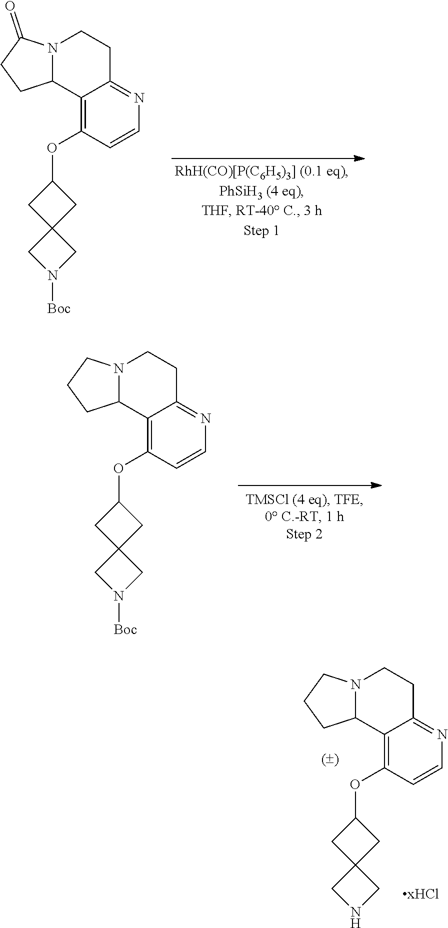 [SMILES: CC(C)(C)OC(=O)N1CC2(CC(Oc3ccnc4c3C3CCC(=O)N3CC4)C2)C1 ; CC(C)(C)OC(=O)N1CC2(CC(Oc3ccnc4c3C3CCCN3CC4)C2)C1 ; c1cc(OC2CC3(CNC3)C2)c2c(n1)CCN1CCCC21]

### Step 1. (±)-tert-Butyl 6-((5,6,8,9,10,10α-hexahydropyrrolo[2,1-f][1,6]naphthyridin-1-yl)oxy)-2-azaspiro[3.3]heptane-2-carboxylate

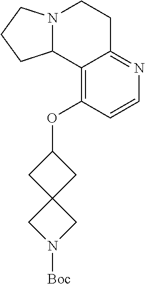 [SMILES: CC(C)(C)OC(=O)N1CC2(CC(Oc3ccnc4c3C3CCCN3CC4)C2)C1]

To a stirred solution of (±)-tert-butyl 6-((8-oxo-5,6,8,9,10,10a-hexahydropyrrolo[2,1-f][1,6]naphthyridin-1-yl)oxy)-2-azaspiro[3.3]heptane-2-carboxylate (19.5 g, 48.8 mmol) in THF (400 mL), carbonylhydridetris(triphenylphosphine)rhodium(I) (4.48 g, 4.88 mmol) was added at RT. To this reaction mixture, phenylsilane (24.06 mL, 195 mmol) was added drop-wise, and the reaction was stirred at 40° C. for 3 h. The reaction progress was monitored by LCMS. After completion, the reaction was poured into ice-cold water (500 mL), and the mixture was extracted with EtOAc (2×200 mL). The combined organic layer was dried over anhydrous sodium sulfate and filtered, and the filtrate was concentrated under reduced pressure. The crude compound was purified by reverse phase chromatography using a SiliCycle SiliaSep C18 column (120 g) on Biotage isolera one (Mobile phase A: 0.1% formic acid in water, B: ACN). The pure fractions were concentrated under reduced pressure. The residue was treated with saturated sodium bicarbonate solution (200 mL) and the mixture was extracted with DCM (3×200 mL). The combined organic layer was dried over sodium sulfate and filtered, and the filtrate was concentrated under reduced pressure to obtain (±)-tert-butyl 6-((5,6,8,9,10,10a-hexahydropyrrolo[2,1-f][1,6]naphthyridin-1-yl)oxy)-2-azaspiro[3.3]heptane-2-carboxylate (10.3 g, 54.4% yield) as a solid. ¹H NMR (400 MHz, DMSO-d₆): δ 8.18 (d, J=5.5 Hz, 1H), 6.66 (d, J=5.6 Hz, 1H), 4.71 (quin, J=6.6 Hz, 1H), 3.91 (br s, 2H), 3.84 (br s, 2H), 3.64 (t, J=8.3 Hz, 1H), 3.02-2.83 (m, 3H), 2.78-2.61 (m, 5H), 2.45-2.36 (m, 1H), 2.29-2.11 (m, 2H), 1.87-1.67 (m, 2H), 1.54-1.41 (m, 1H), 1.37 (s, 9H); LCMS (Method B): Rt=1.11 min, m/z=386.5 [M+H]⁺, 99.43%.

### Step 2. (±)-1-((2-Azaspiro[3.3]heptan-6-yl)oxy)-5,6,8,9,10,10α-hexahydropyrrolo[2,1-f][1,6]naphthyridine xHCl

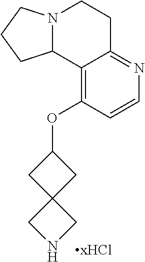 [SMILES: c1cc(OC2CC3(CNC3)C2)c2c(n1)CCN1CCCC21]

To a stirred solution of (±)-tert-butyl 6-((5,6,8,9,10,10a-hexahydropyrrolo[2,1-f][1,6]naphthyridin-1-yl)oxy)-2-azaspiro[3.3]heptane-2-carboxylate (0.95 g, 2.464 mmol) in TFE (10 mL), TMSCl (1.260 mL, 9.86 mmol) was added at 0° C. under a nitrogen atmosphere. The reaction was stirred at RT for 2 h, and the reaction progress was monitored by LCMS and TLC (10% MeOH in DCM). The reaction was concentrated under reduced pressure, and the residue was re-concentrated from EtOAc (20 mL) to obtain crude (±)-1-((2-azaspiro[3.3]heptan-6-yl)oxy)-5,6,8,9,10,10a-hexahydropyrrolo[2,1-f][1,6]naphthyridine xHCl (950 mg, quantitative yield) as a gummy solid. This material was used without further purification. LCMS (Method B): Rt=0.28 min, m/z=286.2 [M+H]⁺, 91.85%.

### Intermediate 20. 5-Fluoro-2-methoxybenzoic Acid

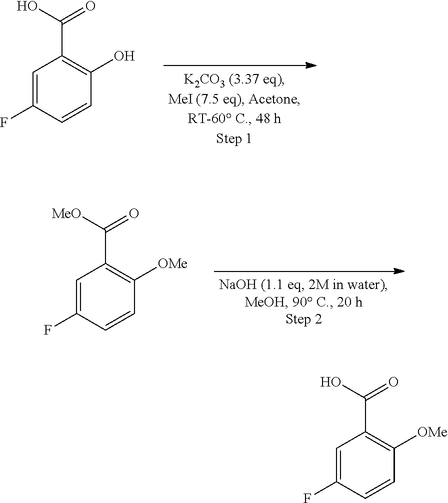 [SMILES: O=C(O)c1cc(F)ccc1O ; COC(=O)c1cc(F)ccc1OC ; COc1ccc(F)cc1C(=O)O]

### Step 1. Methyl 5-fluoro-2-methoxybenzoate

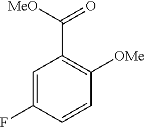 [SMILES: COC(=O)c1cc(F)ccc1OC]

To a dried, 25 L four neck round bottom flask under nitrogen atmosphere, 5-fluoro-2-hydroxybenzoic acid (800 g, 5125 mmol) was dissolved in acetone (7 L). K₂CO₃ (2387 g, 17271 mmol) was added, followed by dropwise addition of MeI (2394 mL, 38437 mmol) at RT. The resulting reaction was heated at 60° C. for 48 h. The reaction progress was monitored by TLC (50% EtOAc in hexane). The reaction was filtered and the filtrate was concentrated under reduced pressure. The residue was diluted with water (4000 mL) and extracted with EtOAc (3×2000 mL). The combined organic layer was dried over anhydrous sodium sulfate and filtered, and the filtrate was concentrated under reduced pressure to obtain methyl 5-fluoro-2-methoxybenzoate (930 g, 97% yield) as a gummy liquid. ¹H NMR (400 MHz, DMSO-d₆): δ 7.47-7.37 (m, 2H), 7.18 (dd, J=4.4, 9.1 Hz, 1H), 3.81 (s, 3H), 3.80 (s, 3H); LCMS (Method L): Rt=1.70 min, m/z=185.2 [M+H]⁺; HPLC (Method D): Rt=5.05 min, 98.07%.

### Step 2. 5-Fluoro-2-methoxybenzoic Acid

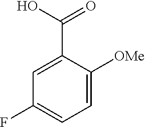 [SMILES: COc1ccc(F)cc1C(=O)O]

In a 25 L four neck round bottom flask, methyl 5-fluoro-2-methoxybenzoate (1.45 kg, 7.87 mol) was dissolved in MeOH (5 L). To this solution, NaOH (4330 mL, 8.66 mol, 2 M in H₂O) was added at RT, and the reaction was stirred at 90° C. for 20 h. The reaction progress was monitored by TLC (50% EtOAc in hexane). The reaction was concentrated, diluted with water (5 L), and acidified to pH 2 with 1.5N HCl (5 L). The resulting solid compound was filtered, washed with water (5 L), and dried under reduced pressure to obtain 5-fluoro-2-methoxybenzoic acid (1.2 kg, 89% yield) as a solid. ¹H NMR (400 MHz, DMSO-d₆): δ 12.85 (br s, 1H), 7.41 (dd, J=3.3, 8.8 Hz, 1H), 7.35 (ddd, J=3.3, 8.1, 9.1 Hz, 1H), 7.14 (dd, J=4.3, 9.1 Hz, 1H), 3.80 (s, 3H); LCMS (Method L): Rt=1.31 min, m/z=169.2 [M−H]⁻, 99.65%.

### Intermediate 21. Methyl 2-((4-chloropyrimidin-5-yl)oxy)-5-fluorobenzoate

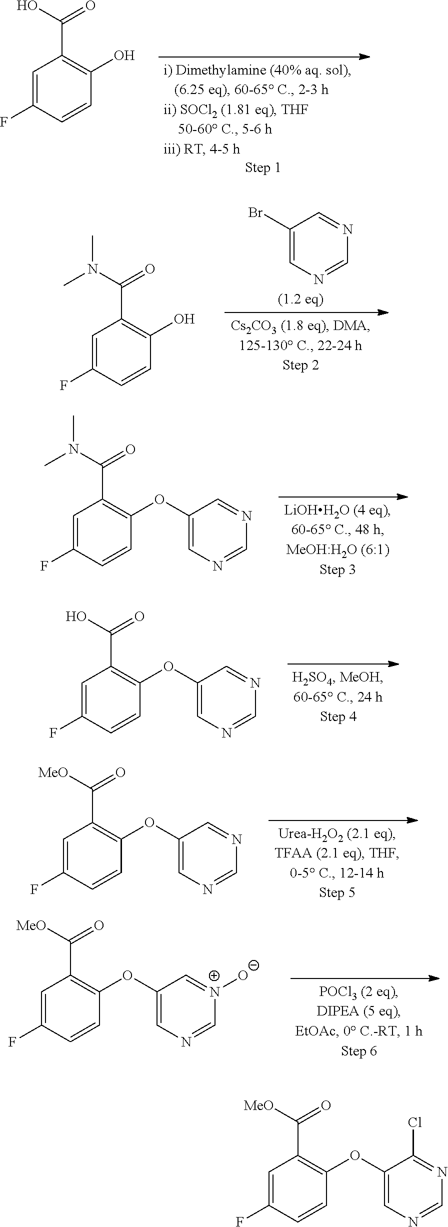

### Step 1. 5-Fluoro-2-hydroxy-N,N-dimethylbenzamide

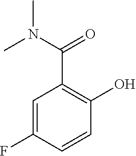

A 40% aqueous dimethylamine solution (140.0 L, 1200.0 mol) was heated at 60-65° C. for 2-3 h to release dimethylamine gas, which was purged into a cooled solution (0-5° C.) of THF (250.0 L).

In another reactor, thionyl chloride (41.0 kg, 347.4 moles) was added to a solution of 5-fluorosalicylic acid (30.0 kg, 192.0 moles) in THF (100.0 L) over a period of 1-2 h. The reaction mixture was heated to 50-60° C. for 5-6 h. After completion of the reaction, as judged by TLC, the reaction mixture was concentrated completely. THF was added and distilled out to remove the remaining thionyl chloride. The acid chloride thus obtained was dissolved in THF (30.0 L) and was added to the dimethylamine-THF solution at 0-5° C. The reaction mixture was stirred at RT for 4-5 h. After completion of the reaction, the reaction mixture was concentrated under a vacuum below 40° C. Ice water was added, and the mixture was stirred for 1-2 h at room temperature to precipitate a solid. The solid was filtered and washed with water. The solid was triturated with cyclohexane and centrifuged to give 5-fluoro-2-hydroxy-N,N-dimethylbenzamide (28.5 kg, 81% yield) as a solid. ¹H NMR (400 MHz, DMSO-d₆): δ 2.81 (s, 3H), 2.94 (s, 3H), 6.83-6.87 (m, 1H), 6.92-6.95 (m, 1H), 7.02-7.07 (m, 1H), 9.76 (br, 1H); HPLC (% purity): 99.63%.

### Step 2. 5-Fluoro-N,N-dimethyl-2-(pyrimidin-5-yloxy)benzamide

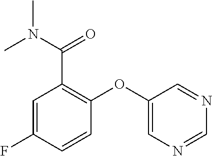 [SMILES: CN(C)C(=O)c1cc(F)ccc1Oc1cncnc1]

To a solution of 5-fluoro-2-hydroxy-N,N-dimethylbenzamide (28.0 kg, 153.0 moles) and 5-bromopyrimidine (29.16 kg, 183.0 moles) in dimethyl acetamide (180.0 L) were added cesium carbonate (89.73, 270.0 moles). The reaction mixture was heated to 125-130° C. for 22-24 h. After complete consumption of the starting material, as judged by TLC, the reaction mixture was cooled to 35-40° C. and filtered to remove the cesium carbonate. The reaction mixture was concentrated under a vacuum. The residue thus obtained was dissolved in water (84.0 L) and extracted with DCM (2×56.0 L). The DCM layer was washed with 10% aqueous sodium hydroxide solution, followed by dilute HCl solution, dried over sodium sulfate, filtered, and concentrated to give 5-fluoro-N,N-dimethyl-2-(pyrimidin-5-yloxy)benzamide (24.0 kg, 60.1% yield) as a semisolid. ¹H NMR (400 MHz, DMSO-d₆): δ 8.94 (s, 1H), 8.54 (s, 2H), 7.25-7.34 (m, 3H), 2.85 (s, 3H), 2.84 (s, 3H); HPLC (% purity): 86.75%.

### Step 3. 5-Fluoro-2-(pyrimidin-5-yloxy)benzoic Acid

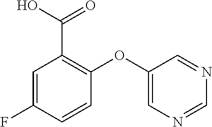 [SMILES: O=C(O)c1cc(F)ccc1Oc1cncnc1]

To a solution of 5-fluoro-N,N-dimethyl-2-(pyrimidin-5-yloxy)benzamide (23.5 kg, 90.03 moles) in 6:1 MeOH/H₂O (140.0 L) was added LiOH·H₂O (15.5 kg, 360.0 moles), and the reaction was heated at 60-65° C. for 48 h. The reaction mixture was cooled to 40° C., and the methanol was distilled out in vacuum below 40° C. The residue thus obtained was dissolved in water. The aqueous layer was washed with ethyl acetate. The aqueous layer was treated with activated carbon and filtered on Hyflo. The aqueous layer was acidified with concentrated HCl (1.5 L) to pH˜1.0-2.0 to precipitate a solid. This solid was filtered, washed with water, and dried at 40-45° C. to give 5-fluoro-2-(pyrimidin-5-yloxy)benzoic acid (13.5 kg, 64.1% yield) as a solid. ¹H NMR (400 MHz, DMSO-d₆): δ 13.40 (br s, 1H), 8.92 (s, 1H), 8.46 (s, 2H), 7.69-7.71 (m, 1H), 7.52-7.57 (m, 1H), 7.38-7.42 (m, 1H); HPLC (% purity): 96.94%.

### Step 4. Methyl 5-fluoro-2-(pyrimidin-5-yloxy)benzoate

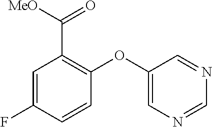 [SMILES: COC(=O)c1cc(F)ccc1Oc1cncnc1]

To a cooled solution of 5-fluoro-2-(pyrimidin-5-yloxy)benzoic acid (13.5 kg, 57.69 moles) in methanol (90.0 L) was added sulfuric acid (2.16 L), and the reaction was heated at 60-65° C. for 24 h. The reaction mixture was cooled to room temperature, and the methanol was distilled out completely under reduced pressure. The residue thus obtained was dissolved in DCM (54.0 L). The DCM layer was washed with 5% sodium bicarbonate solution and saturated sodium chloride solution. The organic layer was dried over anhydrous sodium sulfate, filtered, and concentrated to give methyl 5-fluoro-2-(pyrimidin-5-yloxy)benzoate (9.6 kg, 67.1% yield) as a solid. ¹H NMR (400 MHz, DMSO-d₆): δ 8.94 (s, 1H), 8.50 (s, 2H), 7.72-7.75 (m, 1H), 7.57-7.62 (m, 1H), 7.41-7.45 (m, 1H), 3.73 (s, 3H); HPLC (% purity): 98.37%; assay by Q-NMR (%): 99.21%.

### Step 5. 5-(4-Fluoro-2-(methoxycarbonyl)phenoxy)pyrimidine 1-oxide

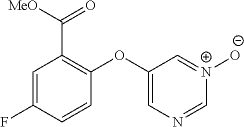 [SMILES: COC(=O)c1cc(F)ccc1Oc1cnc[n+]([O-])c1]

To a cooled solution of methyl 5-fluoro-2-(pyrimidin-5-yloxy)benzoate (9.6 kg, 38.7 moles) in THF (150 L) was added UHP (7.64 kg, 81.2 moles) at 0-5° C. (over a period of 30 min), and the reaction was stirred for 10 min at 0-5° C. TFAA (17.0 kg, 81.0 moles) was then added dropwise over 30-40 min at 0-5° C. The reaction was stirred at 0-5° C. for 12-14 h. After complete consumption of starting material, as judged by TLC, the reaction mixture was quenched by the addition of 5% NaHCO₃ solution at 0-5° C. DCM (60.0 L) was then added at 5-10° C., and the mixture was stirred for 20 min. The aqueous and organic layers were separated. The organic layer was washed with 5% NaHCO₃. Aqueous sodium thiosulfate solution was then added to the organic layer (DCM layer), and the mixture was stirred for 12 hours at 10° C. The organic layer was separated, dried over anhydrous Na₂SO₄, filtered, and concentrated under reduced pressure to give 5-(4-fluoro-2-(methoxycarbonyl)phenoxy)pyrimidine-1-oxide (5.12 kg, 50.0% yield) as solid. ¹HNMR (400 MHz, DMSO-d₆): δ 8.86 (s, 1H), 8.44 (s, 1H), 8.01 (s, 1H), 7.72-7.75 (m, 1H), 7.59-7.64 (m, 1H), 7.49-7.52 (m, 1H), 3.76 (s, 3H); HPLC (% purity): 99.60%.

### Step 6. Methyl 2-((4-chloropyrimidin-5-yl)oxy)-5-fluorobenzoate

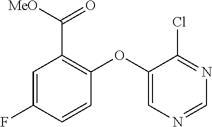 [SMILES: COC(=O)c1cc(F)ccc1Oc1cncnc1Cl]

In a 100 mL two neck round bottom flask under a nitrogen atmosphere, 5-(4-fluoro-2-(methoxycarbonyl)phenoxy)pyrimidine 1-oxide (20 g, 76 mmol) was dissolved in EtOAc (200 mL). To this solution, DIPEA (65.3 mL, 378 mmol) was added at 0° C., and the reaction was stirred at the same temperature for 15 min. To this reaction mixture, POCl₃ (14.15 mL, 151 mmol) was added dropwise at 0° C. The reaction was warmed to RT and stirred for 1 h. The reaction was monitored by TLC (50% EtOAc in hexane). The reaction was concentrated under reduced pressure to afford crude compound. The crude compound was purified by silica gel flash column chromatography using EtOAc in hexane (product eluted at 25% EtOAc in hexane) to obtain methyl 2-((4-chloropyrimidin-5-yl)oxy)-5-fluorobenzoate (16.5 g, 72.2% yield) as a solid. ¹HNMR (400 MHz, DMSO-d₆): δ 8.81 (s, 1H), 8.30 (s, 1H), 7.76 (dd, J=3.1, 8.8 Hz, 1H), 7.61 (ddd, J=3.3, 7.9, 9.0 Hz, 1H), 7.46 (dd, J=4.6, 9.0 Hz, 1H), 3.75 (s, 3H); LCMS (Method A): Rt=1.74 min, m/z=283.1 [M+H]⁺; 93.65%.

### Intermediate 22. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluorobenzoic Acid

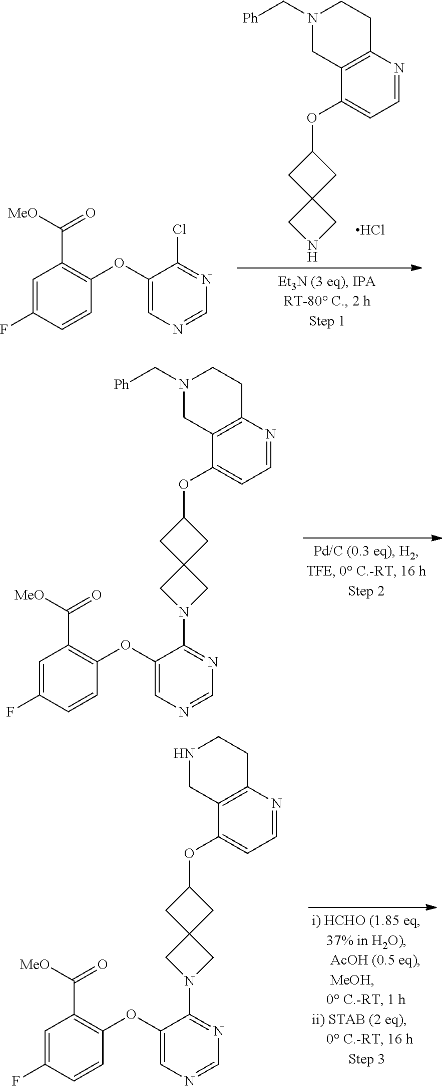

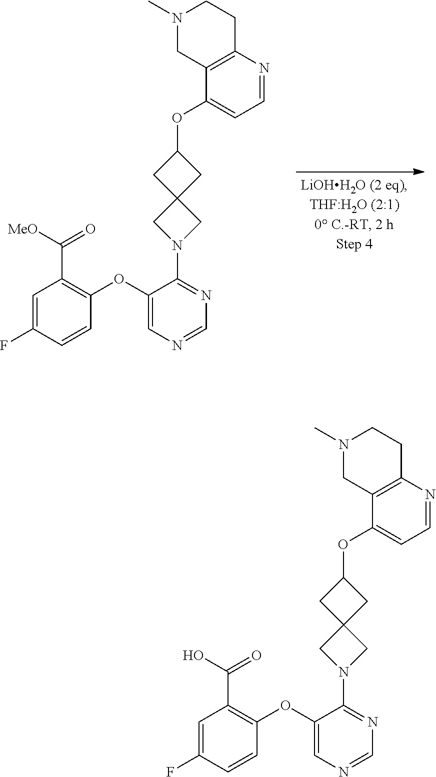 [SMILES: COC(=O)c1cc(F)ccc1Oc1cncnc1N1CC2(CC(Oc3ccnc4c3CN(C)CC4)C2)C1 ; CN1CCc2nccc(OC3CC4(C3)CN(c3ncncc3Oc3ccc(F)cc3C(=O)O)C4)c2C1]

### Step 1. Methyl 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoate

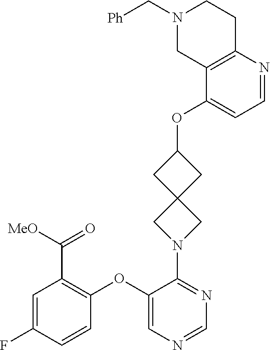 [SMILES: COC(=O)c1cc(F)ccc1Oc1cncnc1N1CC2(CC(Oc3ccnc4c3CN(Cc3ccccc3)CC4)C2)C1]

To a stirred solution of methyl 2-((4-chloropyrimidin-5-yl)oxy)-5-fluorobenzoate (14.93 g, 52.8 mmol) in 2-propanol (150 mL), TEA (22.09 mL, 158 mmol) and 4-((2-azaspiro[3.3]heptan-6-yl)oxy)-6-benzyl-5,6,7,8-tetrahydro-1,6-naphthyridine hydrochloride (19.64 g, 52.8 mmol) were added at RT. The reaction was stirred at 80° C. for 2 h. The reaction progress was monitored by TLC (10% MeOH in DCM). After completion, the reaction was concentrated under reduced pressure, diluted with water (100 mL), and the mixture was extracted with DCM (2×100 mL). The combined organic layer was dried over anhydrous sodium sulfate and filtered, and the filtrate was concentrated under reduced pressure to afford a sticky liquid. The crude product was purified by silica gel flash column chromatography using MeOH in DCM (product eluted at 7% MeOH in DCM) to obtain methyl 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoate (26 g, 82% yield) as a solid ¹H NMR (400 MHz, DMSO-d₆): δ 8.29 (s, 1H), 8.19 (d, J=5.7 Hz, 1H), 7.70-7.63 (m, 2H), 7.49 (ddd, J=3.2, 7.9, 9.0 Hz, 1H), 7.35 (d, J=4.3 Hz, 4H), 7.27 (qd, J=4.2, 8.5 Hz, 1H), 7.11 (dd, J=4.5, 9.2 Hz, 1H), 6.67 (d, J=5.7 Hz, 1H), 4.72 (quin, J=6.6 Hz, 1H), 4.26 (s, 2H), 4.16 (s, 2H), 3.81 (s, 31H), 3.67 (s, 2H), 3.45 (s, 2H), 2.84-2.73 (m, 4H), 2.69 (br t, J=5.2 Hz, 2H), 2.27-2.18 (m, 2H); LCMS (Method F): Rt=2.17 min, m/z=582.4 [M+H]⁺, 97.31%.

### Step 2. Methyl 5-fluoro-2-((4-(6-((5,6,7,8-tetrahydro-1,6-naphthyridin-4-yl)oxy)-2-azaspiro[3.3]heptan-2-yl)pyrimidin-5-yl)oxy)benzoate

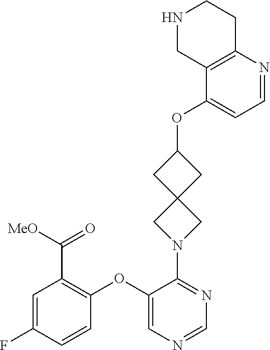 [SMILES: COC(=O)c1cc(F)ccc1Oc1cncnc1N1CC2(CC(Oc3ccnc4c3CNCC4)C2)C1]

To a stirred solution of methyl 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoate (10 g, 17.19 mmol) in TFE (180 mL), Pd—C (5.49 g, 5.16 mmol) was added under a nitrogen atmosphere. The reaction was stirred at RT for 16 h under a hydrogen atmosphere (balloon pressure). The reaction was monitored by TLC(10% MeOH in DCM). After completion, the reaction was filtered through a Celite® pad, and the pad was washed with MeOH (2×100 mL). The filtrate was concentrated under reduced pressure to obtain crude methyl 5-fluoro-2-((4-(6-((5,6,7,8-tetrahydro-1,6-naphthyridin-4-yl)oxy)-2-azaspiro[3.3]heptan-2-yl)pyrimidin-5-yl)oxy)benzoate (8.4 g, 97% yield) as a solid. This material was used without further purification. ¹H NMR (400 MHz, DMSO-d₆): δ 8.30 (s, 1H), 8.17 (d, J=5.6 Hz, 1H), 7.70-7.64 (m, 2H), 7.49 (ddd, J=3.3, 7.9, 9.0 Hz, 11H), 7.10 (dd, J=4.5, 9.1 Hz, 1H), 6.66 (d, J=5.6 Hz, 1H), 4.73 (quin, J=6.6 Hz, 1H), 4.31-4.15 (m, 4H), 3.82 (s, 3H), 3.72 (s, 2H), 2.98 (t, J=5.8 Hz, 2H), 2.83-2.74 (m, 2H), 2.70 (br t, J=5.7 Hz, 2H), 2.30-2.18 (m, 2H), one proton merged with solvent peaks; LCMS (Method A): Rt=1.56 min, m/z=492.2 [M+H]⁺, 97.21%.

### Step 3. Methyl 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoate

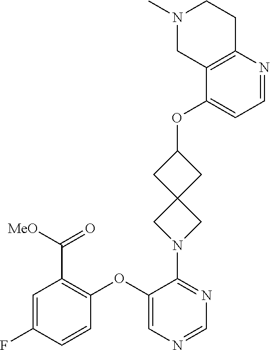 [SMILES: COC(=O)c1cc(F)ccc1Oc1cncnc1N1CC2(CC(Oc3ccnc4c3CN(C)CC4)C2)C1]

In a dried, 500 mL round bottom flask under a nitrogen atmosphere, methyl 5-fluoro-2-((4-(6-((5,6,7,8-tetrahydro-1,6-naphthyridin-4-yl)oxy)-2-azaspiro[3.3]heptan-2-yl)pyrimidin-5-yl)oxy)benzoate (8.4 g, 17.09 mmol) and formaldehyde (2.354 mL, 85 mmol, 37% in H₂O) were dissolved in MeOH (90 mL) at 0° C., then AcOH (0.489 mL, 8.54 mmol) was added. The reaction was stirred at RT for 1 h. To this reaction mixture, sodium triacetoxyborohydride (7.24 g, 34.2 mmol) was added at 0° C., and the reaction was stirred at RT for 16 h. The reaction progress was monitored by TLC (10% MeOH in DCM). After completion, the reaction was concentrated under reduced pressure. Saturated sodium bicarbonate solution (100 mL) was added to the residue and the mixture was extracted with DCM (2×100 mL). The combined organic layer was dried over anhydrous sodium sulfate and filtered, and the filtrate was concentrated under reduced pressure. The crude compound was purified by reverse phase chromatography using a SiliCycle SiliaSep C18 column (120 g) on Biotage isolera one (Mobile phase A: 10 mM NH₄HCO₃ in water, B: ACN). The fractions were concentrated under reduced pressure to afford methyl 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoate as (6.7 g, 75% yield) as white solid. ¹H NMR (400 MHz, DMSO-d₆): δ 8.30 (s, 1H), 8.18 (d, J=5.6 Hz, 1H), 7.69-7.65 (m, 2H), 7.49 (ddd, J=3.3, 7.8, 9.1 Hz, 1H), 7.11 (dd, J=4.4, 9.1 Hz, 1H), 6.67 (d, J=5.8 Hz, 1H), 4.75 (quin, J=6.7 Hz, 1H), 4.27 (s, 2H), 4.18 (s, 2H), 3.82 (s, 3H), 3.35 (s, 2H), 2.84-2.76 (m, 4H), 2.65-2.59 (m, 2H), 2.36 (s, 3H), 2.30-2.23 (m, 2H); LCMS (Method A): Rt=1.69 min, m/z=506.3 [M+H]⁺, 96.85%.

### Step 3. 5-Fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic Acid

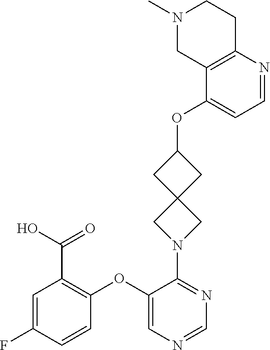 [SMILES: CN1CCc2nccc(OC3CC4(C3)CN(c3ncncc3Oc3ccc(F)cc3C(=O)O)C4)c2C1]

Procedure 1: To a stirred solution of methyl 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoate (7.2 g, 14.24 mmol) in THF:water (55 mL:23.57 mL), lithium hydroxide monohydrate (0.682 g, 28.5 mmol) was added at 0° C. The reaction was stirred at RT for 2 h under a nitrogen atmosphere. The reaction progress was monitored by TLC (10% MeOH in DCM). After completion, the reaction was concentrated under reduced pressure, the crude was dissolved in water (15 mL), and the mixture was acidified with 1.5N HCl (pH˜2). The solvent was concentrated under reduced pressure, and the crude was azeotroped with toluene (2×100 mL) and then with EtOAc (2×100 mL). The residue was lyophilized to afford crude 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid. xHCl (6.2 g, 87% yield) as a solid. This material was used without further purification. ¹H NMR (400 MHz, DMSO-d₆): δ 8.18 (d, J=5.6 Hz, 1H), 8.14 (s, 1H), 7.38 (s, 1H), 7.26 (dd, J=2.5, 9.1 Hz, 1H), 7.05 (dt, J=3.2, 8.5 Hz, 1H), 6.92 (dd, J=4.6, 8.9 Hz, 1H), 6.69 (d, J=5.6 Hz, 1H), 4.76 (quin, J=6.8 Hz, 1H), 4.39-4.30 (m, 2H), 4.29-4.20 (m, 2H), 3.37 (s, 2H), 2.85-2.76 (m, 4H), 2.66-2.60 (m, 2H), 2.36 (s, 3H), 2.30-2.25 (m, 2H), acid proton not observed; LCMS (Method A): Rt=1.91 min, m/z=492.2 [M+H]⁺; HPLC (Method A): Rt=3.42 min, 98.12%.

Procedure 2: To a stirred solution of methyl 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoate (8 g, 15.82 mmol) in THF:water (40 mL:17.14 mL), lithium hydroxide·monohydrate (1.328 g, 31.6 mmol) was added at 0° C. The reaction was stirred at RT for 2 h under a nitrogen atmosphere. The reaction progress was monitored by TLC (10% MeOH in DCM). The reaction was concentrated under reduced pressure, the crude was dissolved in water (20 mL), and the mixture was acidified with 1.5N HCl (pH˜6). The resulting mixture was concentrated under reduced pressure, the residue was dissolved in water (30 mL), and the solution was neutralized with solid NaHCO₃ at 0° C. The precipitated solid was filtered and washed with water (20 mL) to afford 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid (6.1 g, 76% yield) as a solid. ¹H NMR (400 MHz, DMSO-d₆): δ 8.30-8.23 (m, 1H), 8.21 (d, J=5.7 Hz, 1H), 7.61 (s, 1H), 7.55 (dd, J=3.2, 8.7 Hz, 1H), 7.36 (dt, J=3.3, 8.4 Hz, 1H), 7.02 (br dd, J=4.3, 8.9 Hz, 1H), 6.69 (d, J=5.8 Hz, 1H), 4.77 (quin, J=6.2 Hz, 1H), 4.26 (br s, 2H), 4.19 (br s, 2H), 3.53 (s, 2H), 2.91-2.71 (m, 6H), 2.48 (s, 3H), 2.29-2.17 (m, 2H), acid proton not observed; LCMS (Method A): Rt=1.21 min, m/z=492.1 [M+H]⁺, 97.99%.

### Intermediate 23. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxyl-5-fluorobenzoic Acid

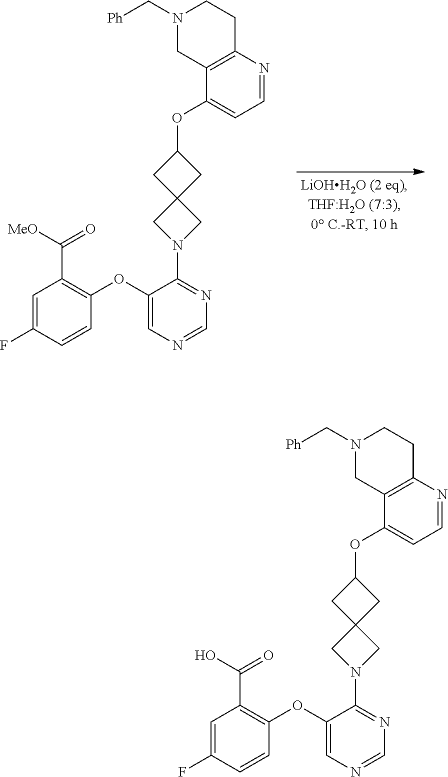 [SMILES: COC(=O)c1cc(F)ccc1Oc1cncnc1N1CC2(CC(Oc3ccnc4c3CN(Cc3ccccc3)CC4)C2)C1 ; O=C(O)c1cc(F)ccc1Oc1cncnc1N1CC2(CC(Oc3ccnc4c3CN(Cc3ccccc3)CC4)C2)C1]

Procedure 1. Lithium hydroxide monohydrate (2.164 g, 51.6 mmol) was added to a stirred solution of methyl 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoate (15 g, 25.8 mmol) in a mixture of THF (150 mL) and water (75 mL) at 0° C. The reaction was stirred at RT for 2 h. The reaction progress was monitored by TLC (10% MeOH in DCM). After completion, the reaction was concentrated under reduced pressure. The crude was acidified with 1.5N HCl (pH-2-3). The mixture was concentrated under reduced pressure. The reside was reconcentrated from toluene (2×50 mL), then from EtOAc (50 mL) to obtain crude 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid xHCl (12 g, 82% yield) as a solid. This material was used without further purification. ¹H NMR (400 MHz, DMSO-d₆): δ 8.26 (s, 1H), 8.19 (d, J=5.8 Hz, 1H), 7.62 (dd, J=3.3, 8.8 Hz, 1H), 7.59 (s, 1H), 7.48-7.39 (m, 1H), 7.38-7.31 (m, 4H), 7.31-7.24 (m, 1H), 7.09 (dd, J=4.5, 9.0 Hz, 1H), 6.68 (d, J=5.8 Hz, 1H), 4.73 (quin, 1=6.7 Hz, 1H), 4.27 (br s, 2H), 4.17 (br s, 2H), 3.70 (s, 2H), 3.48 (s, 2H), 2.85-2.66 (m, 6H), 2.26-2.14 (m, 2H), acidic protons were not observed; LCMS (Method G): Rt=1.37 min, m/z=568.2 [M+H]⁺, 99.28%.

Procedure 2. To a stirred solution of methyl 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoate (20 g, 34.4 mmol) in THF:H₂O (7:3, 140 mL), lithium hydroxide monohydrate (4.33 g, 103 mmol) was added at 0° C. The reaction was stirred at RT for 10 h. The reaction progress was monitored by TLC (10% MeOH in DCM). After completion, the reaction was concentrated under reduced pressure. The residue was neutralized with 1.5N HCl (pH˜7) at 0° C. and the mixture was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN) to obtain 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid (12 g, 57.8% yield) as a solid. ¹H NMR (400 MHz, DMSO-d₆): δ 8.21-8.16 (m, 2H), 7.50-7.31 (m, 6H), 7.29-7.24 (m, 1H), 7.22-7.13 (m, 1H), 7.05-6.89 (m, 1H), 6.68 (d, J=5.6 Hz, 1H), 4.73 (quin, J=6.6 Hz, 1H), 4.31 (br s, 2H), 4.21 (br s, 2H), 3.68 (s, 2H), 2.84-2.73 (m, 4H), 2.72-2.68 (m, 2H), 2.27-2.18 (m, 2H), three protons merged with solvent peaks; LCMS (Method A): Rt=1.40 min, m/z=568.1 [M+H]⁺, 98.33%.

### Intermediate 24. 2-((4-(6-((6-(tert-Butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic Acid

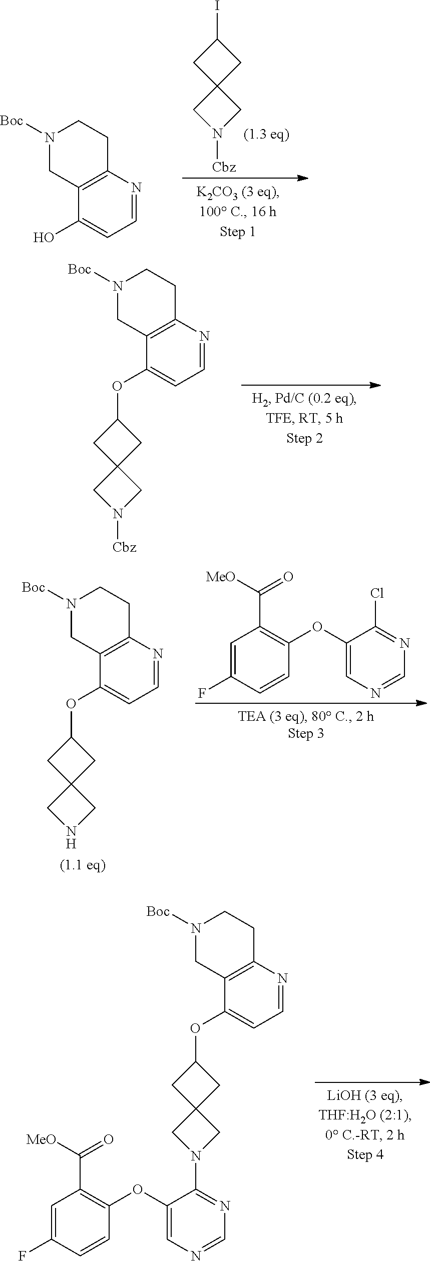

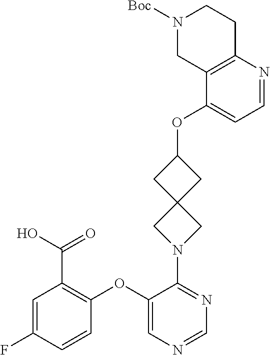 [SMILES: CC(C)(C)OC(=O)N1CCc2nccc(OC3CC4(C3)CN(c3ncncc3Oc3ccc(F)cc3C(=O)O)C4)c2C1]

### Step 1. tert-Butyl 4-((2-((benzyloxy)carbonyl)-2-azaspiro[3.3]heptan-6-yl)oxy)-7,8-dihydro-1,6-naphthyridine-6(5H)-carboxylate

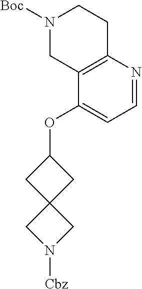 [SMILES: CC(C)(C)OC(=O)N1CCc2nccc(OC3CC4(C3)CN(C(=O)OCc3ccccc3)C4)c2C1]

To a stirred solution of tert-butyl 4-hydroxy-7,8-dihydro-1,6-naphthyridine-6(5-t)-carboxylate (47 g, 188 mmol) in DMF (750 mL), benzyl 6-iodo-2-azaspiro[3.3]heptane-2-carboxylate (87 g, 244 mmol), and K₂CO₃ (78 g, 563 mmol) were added at RT. The reaction was stirred at 100° C. for 16 h. The reaction progress was monitored by TLC (100% EtOAc). After completion, the reaction was quenched with water (1 L) and the mixture was extracted with EtOAc (2×1.5 L). The combined organic layer was washed with brine (2×1.5 L), dried over sodium sulfate, and filtered, and the filtrate was concentrated under reduced pressure. The crude was purified by silica gel flash column chromatography using MeOH in DCM (product eluted at 10% MeOH in DCM) to obtain tert-butyl 4-((2-((benzyloxy)carbonyl)-2-azaspiro[3.3]heptan-6-yl)oxy)-7,8-dihydro-1,6-naphthyridine-6(5H)-carboxylate (71.82 g, 77% yield) as a gummy liquid. ¹H NMR (400 MHz, DMSO-d₆): δ 8.24 (d, J=5.6 Hz, 1H), 7.44-7.28 (m, 5H), 6.73 (d, J=5.8 Hz, 1H), 5.03 (s, 2H), 4.76 (quin, J=6.3 Hz, 1H), 4.36 (s, 2H), 4.14-3.88 (m, 4H), 3.62 (br t, J=5.8 Hz, 2H), 2.84-2.72 (m, 4H), 2.30-2.21 (m, 2H), 1.42 (s, 9H); LCMS (Method A): Rt=2.23 min, m/z=480.4 [M+H]⁺, 96.28%.

### Step 2. tert-Butyl 4-((2-azaspiro[3.3]heptan-6-yl)oxy)-7,8-dihydro-1,6-naphthyridine-6(5H)-carboxylate

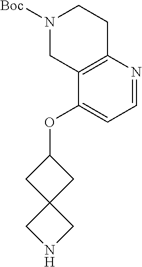 [SMILES: CC(C)(C)OC(=O)N1CCc2nccc(OC3CC4(CNC4)C3)c2C1]

In a 250 mL round bottom flask under a nitrogen atmosphere, tert-butyl 4-((2-((benzyloxy)carbonyl)-2-azaspiro[3.3]heptan-6-yl)oxy)-7,8-dihydro-1,6-naphthyridine-6(5H)-carboxylate (5 g, 10.43 mmol) was dissolved in TFE (50 mL). To this solution, Pd—C (2.219 g, 2.085 mmol) was added and the reaction was stirred at RT for 5 h under a hydrogen atmosphere (balloon pressure). The reaction progress was monitored by TLC (10% MeOH in DCM). After completion, the reaction was filtered through a Celite® pad, and the pad was washed with MeOH (80 mL). The filtrate was concentrated under reduced pressure to obtain crude tert-butyl 4-((2-azaspiro[3.3]heptan-6-yl)oxy)-7,8-dihydro-1,6-naphthyridine-6(5H)-carboxylate (3.5 g, 74.4% yield) as a gummy liquid. This material was used without further purification. LCMS (Method A): Rt=1.44 min, m/z=346.1 [M+H]⁺, 76.51%.

### Step 3. tert-Butyl 4-((2-(5-(4-fluoro-2-(methoxycarbonyl)phenoxy)pyrimidin-4-yl)-2-azaspiro[3.3]heptan-6-yl)oxy)-7,8-dihydro-1,6-naphthyridine-6(5H)-carboxylate

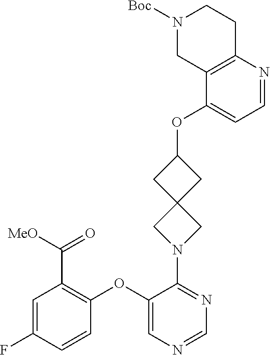 [SMILES: COC(=O)c1cc(F)ccc1Oc1cncnc1N1CC2(CC(Oc3ccnc4c3CN(C(=O)OC(C)(C)C)CC4)C2)C1]

To a stirred solution of tert-butyl 4-((2-azaspiro[3.3]heptan-6-yl)oxy)-7,8-dihydro-1,6-naphthyridine-6(5H)-carboxylate (2.017 g, 5.84 mmol) in 2-propanol (20 mL), TEA (2.219 mL, 15.92 mmol), and methyl 2-((4-chloropyrimidin-5-yl)oxy)-5-fluorobenzoate (1.5 g, 5.31 mmol) were added at RT. The reaction was stirred at 80° C. for 2 h. The reaction progress was monitored by TLC (100% EtOAc). When complete, the reaction was concentrated under reduced pressure. The crude was purified by silica gel flash column chromatography using MeOH in DCM (product eluted at 5-10% MeOH in DCM) to obtain tert-butyl 4-((2-(5-(4-fluoro-2-(methoxycarbonyl)phenoxy)pyrimidin-4-yl)-2-azaspiro[3.3]heptan-6-yl)oxy)-7,8-dihydro-1,6-naphthyridine-6(5H)-carboxylate (1.9 g, 50.1% yield) as a solid. ¹H NMR (400 MHz, CDCl₃): δ 8.38 (s, 1H), 8.28 (d, J=5.7 Hz, 1H), 7.70-7.62 (m, 2H), 7.26-7.16 (m, 1H), 6.88 (dd, J=4.3, 9.1 Hz, 1H), 6.46 (d, J=5.8 Hz, 1H), 4.67 (quin, J=6.5 Hz, 1H), 4.48 (br s, 2H), 4.35 (s, 2H), 4.32 (s, 2H), 3.90 (s, 3H), 3.71 (br t, 0.1=5.8 Hz, 2H), 2.95 (br t, J=5.6 Hz, 2H), 2.87-2.77 (m, 2H), 2.50-2.36 (m, 2H), 1.50 (s, 9H); LCMS (Method A): Rt=2.07 min, m/z=592.2 [M+H]⁺, 82.78%.

### Step 4. 2-((4-(6-((6-(tert-Butoxycarbonyl)-5,6,7,8-tetrahydro-1,6-naphthyridin-4-yl)oxy)-2-azaspiro[3.3]heptan-2-yl)pyrimidin-5-yl)(oxy)-5-fluorobenzoic Acid

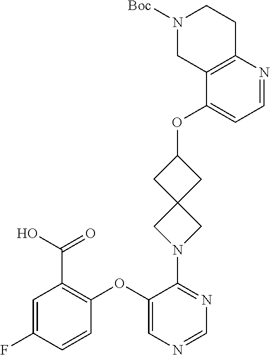 [SMILES: CC(C)(C)OC(=O)N1CCc2nccc(OC3CC4(C3)CN(c3ncncc3Oc3ccc(F)cc3C(=O)O)C4)c2C1]

To a stirred solution of tert-butyl 4-((2-(5-(4-fluoro-2-(methoxycarbonyl)phenoxy)pyrimidin-4-yl)-2-azaspiro[3.3]heptan-6-yl)oxy)-7,8-dihydro-1,6-naphthyridine-6(5H)-carboxylate (1.9 g, 3.21 mmol) in THF:H₂O (2:1, 21 mL), LiOH·H₂O (0.404 g, 9.63 mmol) was added at 0° C. The reaction was stirred at RT for 2 h. The reaction progress was monitored by TLC (10% MeOH in DCM). After completion, the reaction was concentrated and the residue was reconcentrated from toluene (3×15 mL) to afford crude lithium 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoate (1.8 g, 88% yield) as a solid. The crude material (1 g, 1.714 mmol) was diluted with water (10 mL), cooled to 0° C., and neutralized with 1.5N HCl (2.285 mL, 3.43 mmol) to pH˜7. The mixture was extracted with EtOAc (2×20 mL). The combined organic layer was dried over sodium sulfate and filtered, and the filtrate was concentrated under reduced pressure to obtain crude 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid (380 mg, 37.7% yield) as a solid. This material was used without further purification. ¹H NMR (400 MHz, DMSO-d₆): δ 13.32 (br s, 1H), 8.26 (s, 1H), 8.23 (d, J=5.6 Hz, 1H), 7.64-7.59 (m, 1H), 7.58 (s, 1H), 7.47-7.39 (m, 1H), 7.11 (dd, J=4.5, 9.1 Hz, 1H), 6.75 (d, J=5.8 Hz, 1H), 4.79 (quin, J=6.5 Hz, 1H), 4.36 (s, 2H), 4.30 (br s, 2H), 4.22 (br s, 2H), 3.61 (br t, J=5.8 Hz, 2H), 2.86-2.76 (m, 4H), 2.32-2.26 (m, 2H), 1.42 (s, 9H); LCMS (Method G): Rt=1.54 min, m/z=578.3 [M+H]⁺, 98.27%.

### Intermediate 25. (±)-2-((4-(6-((6-Benzyl-5-methyl-5,6,7,8-tetrahydro-1,6-naphthyridin-4-yl)oxy)-2-azaspiro[3.3]heptan-2-yl)pyrimidin-5-yl)oxy)-5-fluorobenzoic Acid

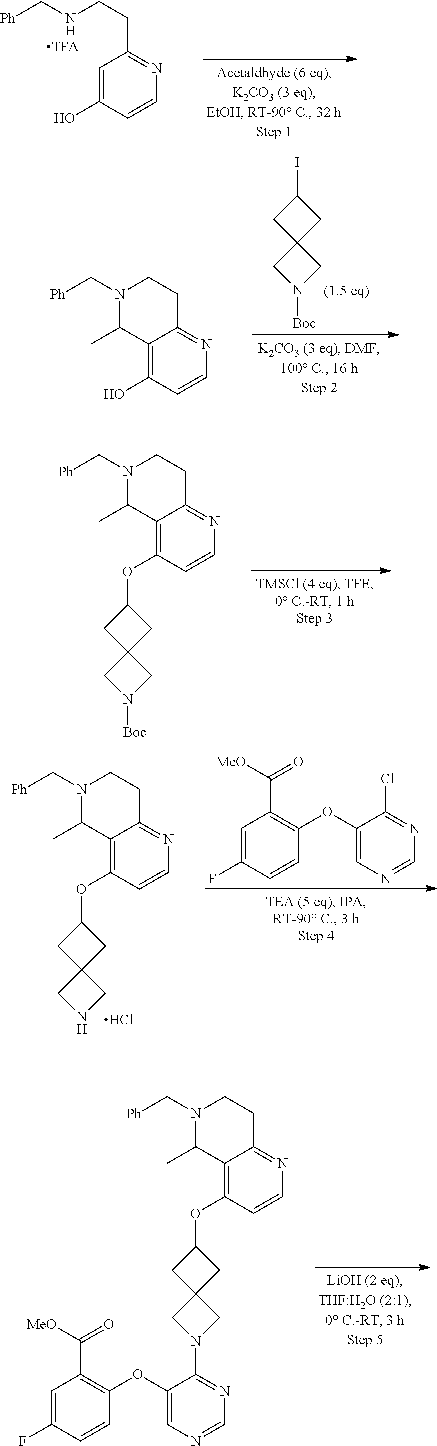

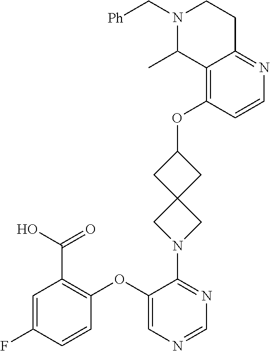 [SMILES: CC1c2c(OC3CC4(C3)CN(c3ncncc3Oc3ccc(F)cc3C(=O)O)C4)ccnc2CCN1Cc1ccccc1]

### Step 1. (±)-6-Benzyl-5-methyl-5,6,7,8-tetrahydro-1,6-naphthyridin-4-ol

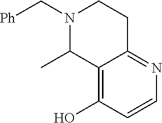 [SMILES: CC1c2c(O)ccnc2CCN1Cc1ccccc1]

In a 500 mL two neck round bottom flask under a nitrogen atmosphere, 2-(2-(benzylamino)ethyl)pyridin-4-ol 2,2,2-trifluoroacetate (10 g, 29.2 mmol) was dissolved in EtOH (100 mL). To this solution, K₂CO₃ (12.11 g, 88 mmol) and acetaldehyde (4.95 mL, 88 mmol) were added at RT and the reaction was stirred at 90° C. for 16 h. The reaction progress was monitored by TLC (10% MeOH in DCM). The starting material was not consumed, therefore another portion of acetaldehyde (4.95 mL, 88 mmol) was added and the reaction was stirred at 90° C. for 16 h. After completion, the reaction was filtered through a Celite® pad, and the pad was washed with MeOH (100 mL). The filtrate was concentrated under reduced pressure and the crude was purified by silica gel flash column chromatography using MeOH in DCM (product was eluted at 20% MeOH in DCM) to obtain (±)-6-benzyl-5-methyl-5,6,7,8-tetrahydro-1,6-naphthyridin-4-ol (4.5 g, 57.6% yield) as a viscous liquid. LCMS (Method A): Rt=1.72 min, m/z=255.1 [M+H]⁺, 95.12%.

### Step 2. (±)-tert-Butyl 6-((6-benzyl-5-methyl-5,6,7,8-tetrahydro-1,6-naphthyridin-4-yl)oxy)-2-azaspiro[3.3]heptane-2-carboxylate

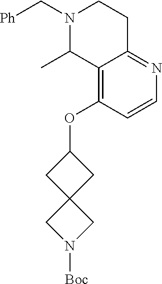 [SMILES: CC1c2c(OC3CC4(C3)CN(C(=O)OC(C)(C)C)C4)ccnc2CCN1Cc1ccccc1]

To a stirred solution of (±)-6-benzyl-5-methyl-5,6,7,8-tetrahydro-1,6-naphthyridin-4-ol (4.2 g, 16.51 mmol) in DMF (45 mL), K₂CO₃ (6.85 g, 49.5 mmol) and tert-butyl 6-iodo-2-azaspiro[3.3]heptane-2-carboxylate (8.01 g, 24.77 mmol) were added at RT. The reaction was stirred at 100° C. for 16 h. The reaction progress was monitored by TLC (10% MeOH in DCM). After completion, the reaction was quenched with ice cold water (50 mL), and the mixture was extracted with EtOAc (3×60 mL). The combined organic layer was washed with brine (50 mL), dried over sodium sulfate and filtered, and the filtrate was concentrated under reduced pressure. The crude was purified by silica gel flash column chromatography using MeOH in DCM (product was eluted at 7% MeOH in DCM) to obtain (f)-tert-butyl 6-((6-benzyl-5-methyl-5,6,7,8-tetrahydro-1,6-naphthyridin-4-yl)oxy)-2-azaspiro[3.3]heptane-2-carboxylate (4.2 g, 53.2% yield) as a gummy liquid. ¹H NMR (400 MHz, DMSO-d₆): δ 8.20 (d, J=5.6 Hz, 1H), 7.39-7.23 (m, 5H), 6.66 (d, J=5.6 Hz, 1H), 4.75-4.65 (m, 1H), 4.02-3.93 (m, 1H), 3.92-3.86 (m, 2H), 3.83-3.80 (m, 2H), 3.78-3.74 (m, 1H), 3.61 (d, J=13.9 Hz, 1H), 3.09-3.01 (m, 1H), 2.88-2.82 (m, 1H), 2.73-2.69 (m, 2H), 2.20-2.11 (m, 2H), 1.37 (s, 9H), 1.24 (d, J=6.6 Hz, 3H), two protons merged with solvent peaks; LCMS (Method A): Rt=2.27 min, m/z=450.2 [M+H]⁺, 94.07%.

### Step 3. (±)-4-((2-Azaspiro[3.3]heptan-6-yl)oxy)-6-benzyl-5-methyl-5,6,7,8-tetrahydro-1,6-naphthyridine Hydrochloride

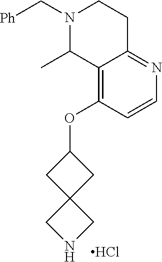 [SMILES: CC1c2c(OC3CC4(CNC4)C3)ccnc2CCN1Cc1ccccc1 ; Cl]

To a stirred solution of (f)-tert-butyl 6-((6-benzyl-5-methyl-5,6,7,8-tetrahydro-1,6-naphthyridin-4-yl)oxy)-2-azaspiro[3.3]heptane-2-carboxylate (4.2 g, 9.34 mmol) in TFE (45 mL), TMSCl (4.78 mL, 37.4 mmol) was added at 0° C. The reaction was stirred at RT for 1 h. The reaction progress was monitored by TLC (10% MeOH in DCM). After completion, the reaction was concentrated under reduced pressure, and the residue was triturated with EtOAc (2×50 mL). The organic layer was decanted and the resulting solid was dried under vacuum to afford (±)-4-((2-azaspiro[3.3]heptan-6-yl)oxy)-6-benzyl-5-methyl-5,6,7,8-tetrahydro-1,6-naphthyridine hydrochloride (4.5 g, quantitative yield) as a solid. LCMS (Method A): Rt=1.50 min, m/z=350.1 [M+H]⁺, 96.73%.

### Step 4. (±)-Methyl 2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluorobenzoate

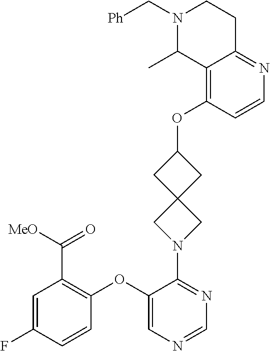 [SMILES: COC(=O)c1cc(F)ccc1Oc1cncnc1N1CC2(CC(Oc3ccnc4c3C(C)N(Cc3ccccc3)CC4)C2)C1]

In a 250 mL round bottom flask under a nitrogen atmosphere, (±)-4-((2-azaspiro[3.3]heptan-6-yl)oxy)-6-benzyl-5-methyl-5,6,7,8-tetrahydro-1,6-naphthyridine hydrochloride (4.5 g, 11.66 mmol) and methyl 2-((4-chloropyrimidin-5-yl)oxy)-5-fluorobenzoate (3.30 g, 11.66 mmol) were dissolved in IPA (45 mL). To this reaction mixture, TEA (8.13 mL, 58.3 mmol) was added at RT and the reaction was stirred at 90° C. for 3 h. The reaction progress was monitored by TLC (10% MeOH in DCM). After completion, the reaction was concentrated under reduced pressure, the residue was diluted with water (40 mL), and the mixture was extracted with EtOAc (3×30 mL). The combined organic layer was washed with brine (50 mL), dried over sodium sulfate and filtered, and the filtrate was concentrated under reduced pressure. The crude was purified by silica gel flash column chromatography using MeOH in DCM (product was eluted at 8% MeOH in DCM) to obtain (±)-methyl 2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluorobenzoate (4.2 g, 53.7% yield) as a gummy solid. ¹H NMR (400 MHz, DMSO-d₆): δ 8.30 (s, 1H), 8.20 (d, J=5.5 Hz, 1H), 7.72-7.62 (m, 2H), 7.49 (ddd, J=3.3, 7.9, 9.0 Hz, 1H), 7.38-7.21 (m, 5H), 7.11 (dd, J=4.4, 9.1 Hz, 1H), 6.68 (d, J=5.7 Hz, 1H), 4.74 (quin, J=6.6 Hz, 1H), 4.26 (s, 2H), 4.17 (s, 2H), 4.02-3.92 (m, 1H), 3.81 (s, 3H), 3.78-3.72 (m, 1H), 3.63-3.57 (m, 1H), 3.13-3.00 (m, 1H), 2.94-2.68 (m, 4H), 2.64-2.56 (m, 1H), 2.30-2.14 (m, 2H), 1.23 (d, J=6.4 Hz, 3H); LCMS (Method A): Rt=2.16 min, m/z=596.1 [M+H]⁺, 88.72%.

### Step 5. (±)-2-((4-(6-((6-Benzyl-5-methyl-5,6,7,8-tetrahydro-1,6-naphthyridin-4-yl)oxy)-2-azaspiro[3.3]heptan-2-yl)pyrimidin-5-yl)oxy)-5-fluorobenzoic Acid

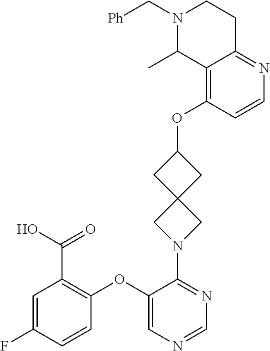 [SMILES: CC1c2c(OC3CC4(C3)CN(c3ncncc3Oc3ccc(F)cc3C(=O)O)C4)ccnc2CCN1Cc1ccccc1]

To a stirred solution of (±)-methyl 2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluorobenzoate (1 g, 1.679 mmol) in THF:H₂O (2:1, 15 mL), LiOH·H₂O (0.14 g, 3.36 mmol) was added at 0° C. The reaction was stirred at RT for 3 h. The reaction progress was monitored by TLC (10% MeOH in DCM). After completion, the reaction was concentrated under reduced pressure. The residue was acidified with 1.5N HCl (pH˜5) and the mixture was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN) to obtain (±)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluorobenzoic acid (900 mg, 92% yield) as a solid. ¹H NMR (400 MHz, DMSO-d₆): δ; LCMS (Method A): Rt=1.43 min, m/z=582.2 [M+H]⁺, 91.65%.

### Intermediate 26. tert-Butyl ((1s,3s)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclobutyl)carbamate

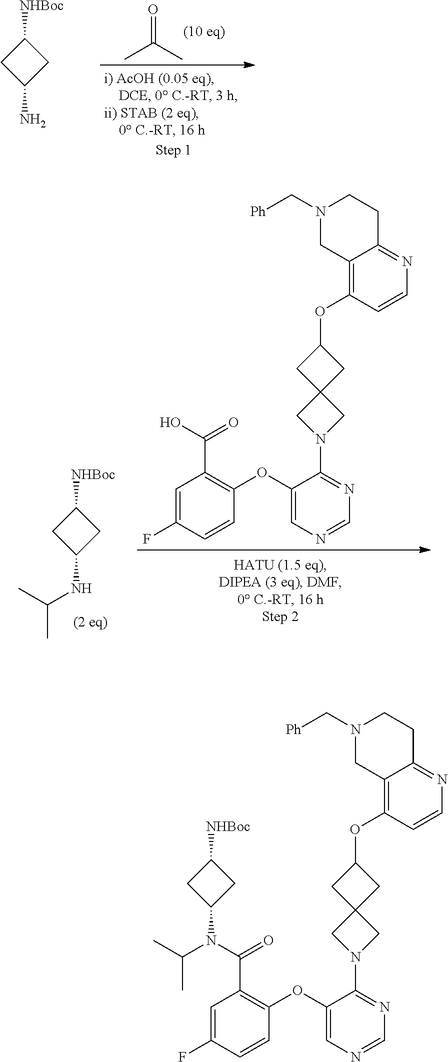

### Step 1. tert-Butyl ((1s,3s)-3-(isopropylamino)cyclobutyl)carbamate

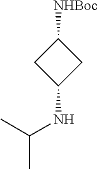 [SMILES: CC(C)N[C@H]1C[C@@H](NC(=O)OC(C)(C)C)C1]

This compound was synthesized following the procedure described for the synthesis of Intermediate 1, starting with 1 equivalent of tert-butyl ((1s,3s)-3-aminocyclobutyl)carbamate, except that 10 equivalents of acetone was used, and DCE was used as the solvent. After work-up, the filtrate was concentrated under reduced pressure to obtain crude tert-butyl ((1s,3s)-3-(isopropylamino)cyclobutyl)carbamate (40.0% yield) as a semi-solid. This material was used without further purification. ¹H NMR (400 MHz, CDCl₃): δ 4.64 (br s, 1H), 3.89-3.77 (m, 1H), 3.07-2.98 (m, 1H), 2.87-2.77 (m, 1H), 2.77-2.64 (m, 2H), 1.62-1.50 (m, 2H), 1.45 (s, 9H), 1.04 (d, J=6.3 Hz, 6H), one proton merged with solvent peaks.

### Step 2. tert-Butyl ((1s, 3s)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclobutyl)carbamate

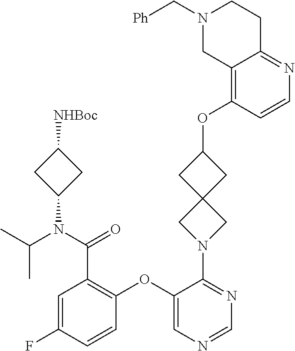 [SMILES: CC(C)N(C(=O)c1cc(F)ccc1Oc1cncnc1N1CC2(CC(Oc3ccnc4c3CN(Cc3ccccc3)CC4)C2)C1)[C@H]1C[C@@H](NC(=O)OC(C)(C)C)C1]

In a dried, 50 mL two neck round bottom flask under a nitrogen atmosphere, 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid (0.400 g, 0.705 mmol) and tert-butyl ((1s,3s)-3-(isopropylamino)cyclobutyl)carbamate (0.322 g, 1.409 mmol) were dissolved in DMF (5 mL). To this solution, DIPEA (0.358 mL, 2.114 mmol) and HATU (0.402 g, 1.057 mmol) were added at 0° C. and the reaction was stirred at RT for 16 h. The reaction progress was monitored by TLC (10% MeOH in DCM). After completion, the reaction was diluted with cold water (25 mL) and the mixture was extracted with EtOAc (3×30 mL). The combined organic layer was washed with brine solution (30 mL), dried over anhydrous sodium sulfate, and filtered, and the filtrate was concentrated under reduced pressure. The crude product was purified by silica gel flash column chromatography using MeOH in DCM (product eluted at 10% MeOH in DCM) to obtain tert-butyl ((1s,3s)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclobutyl)carbamate (0.350 g, 54.9% yield). LCMS (Method A): Rt=2.34 min, m/z=778.9 [M+H]⁺, 86.33%.

### Intermediate 27. tert-Butyl ((1r,3r)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclobutyl)carbamate

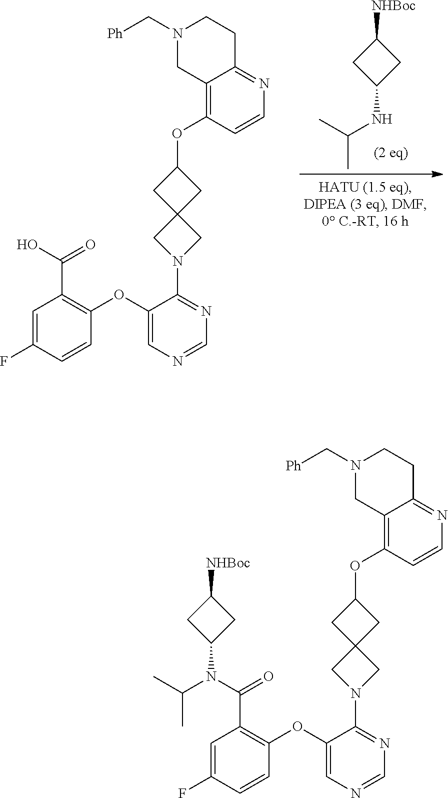 [SMILES: O=C(O)c1cc(F)ccc1Oc1cncnc1N1CC2(CC(Oc3ccnc4c3CN(Cc3ccccc3)CC4)C2)C1 ; CC(C)N[C@H]1C[C@H](NC(=O)OC(C)(C)C)C1 ; CC(C)N(C(=O)c1cc(F)ccc1Oc1cncnc1N1CC2(CC(Oc3ccnc4c3CN(Cc3ccccc3)CC4)C2)C1)[C@H]1C[C@H](NC(=O)OC(C)(C)C)C1]

This compound was synthesized following the procedure described for the synthesis of Intermediate 26, step 2, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 2 equivalents of tert-butyl ((1r,3r)-3-(isopropylamino)cyclobutyl)carbamate. After work-up, the crude product was purified by silica gel flash column chromatography using MeOH in DCM (product eluted at 10% MeOH in DCM) to obtain tert-butyl ((1r,3r)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclobutyl)carbamate as a solid.

Yield: 54.9%; LCMS (Method A): Rt=2.32 min, m/z=778.8 [M+H]⁺, 85.07%.

### Intermediate 28. N-((1r,3r)-3-Aminocyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl

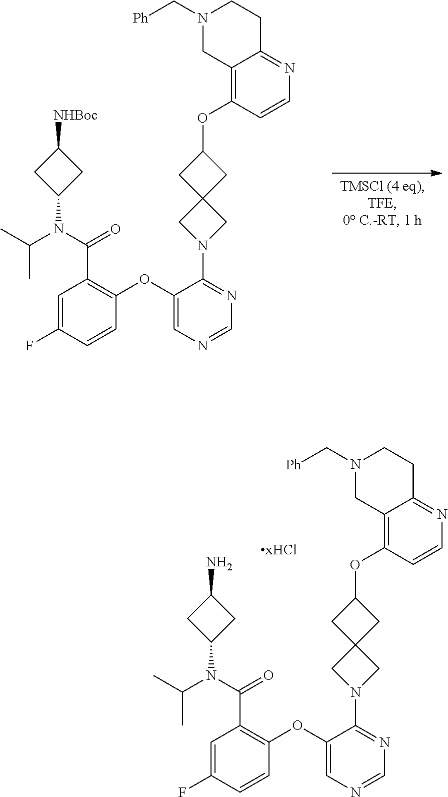 [SMILES: CC(C)N(C(=O)c1cc(F)ccc1Oc1cncnc1N1CC2(CC(Oc3ccnc4c3CN(Cc3ccccc3)CC4)C2)C1)[C@H]1C[C@H](NC(=O)OC(C)(C)C)C1 ; CC(C)N(C(=O)c1cc(F)ccc1Oc1cncnc1N1CC2(CC(Oc3ccnc4c3CN(Cc3ccccc3)CC4)C2)C1)[C@H]1C[C@H](N)C1]

To a stirred solution of tert-butyl ((1r,3r)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclobutyl)carbamate (0.450 g, 0.578 mmol) in 2,2,2-trifluoroethanol (5 mL), TMSCl (0.296 mL, 2.314 mmol) was added at 0° C. The reaction was stirred at RT for 1 h. The reaction progress was monitored by LCMS. After completion, the reaction was concentrated under reduced pressure to obtain crude N-((1r,3r)-3-aminocyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl (0.410 g, 94% yield) as a solid. This material was used without further purification. LCMS (Method F): Rt=2.03 min, m/z=678.5 [M+H]⁺, 95.07%.

### Intermediate 29. tert-Butyl (3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)bicyclo[1.1.1]pentan-1-yl)carbamate

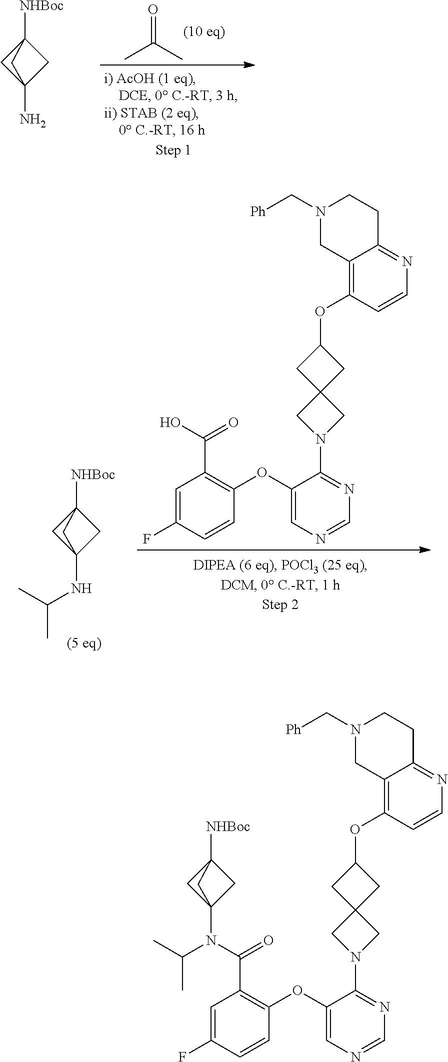

### Step 1. tert-Butyl (3-(Isopropylamino)bicyclo[1.1.1]pentan-1-yl)carbamate

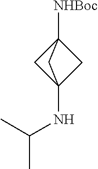 [SMILES: CC(C)NC12CC(NC(=O)OC(C)(C)C)(C1)C2]

This compound was synthesized following the procedure described for the synthesis of Intermediate 1, starting with 1 equivalent of tert-butyl (3-aminobicyclo[1.1.1]pentan-1-yl)carbamate, except that 10 equivalents of acetone and 1 equivalent of AcOH were used, and DCE was used as the solvent. After work-up, the filtrate was concentrated under reduced pressure to obtain crude tert-butyl (3-(isopropylamino)bicyclo[1.1.1]pentan-1-yl)carbamate as a semi-solid. This material was used without further purification.

Yield: 50.7%; ¹H NMR (400 MHz, DMSO-d₆): δ 7.38 (br s, 1H), 2.78-2.66 (m, 1H), 1.84 (s, 6H), 1.41-1.33 (m, 9H), 0.95 (d, J=6.2 Hz, 6H), one proton was merged with solvent peaks.

### Step 2. tert-Butyl (3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)bicyclo[1.1.1]pentan-1-yl)carbamate

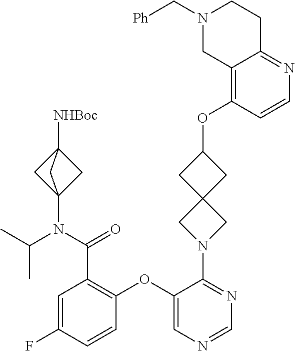 [SMILES: CC(C)N(C(=O)c1cc(F)ccc1Oc1cncnc1N1CC2(CC(Oc3ccnc4c3CN(Cc3ccccc3)CC4)C2)C1)C12CC(NC(=O)OC(C)(C)C)(C1)C2]

To a stirred solution of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid (1 g, 1.762 mmol) in DCM (30 mL), tert-butyl (3-(isopropylamino)bicyclo[1.1.1]pentan-1-yl)carbamate (2.117 g, 8.81 mmol) and DIPEA (7.69 mL, 44.0 mmol) were added at 0° C. To this reaction mixture, POCl₃ (0.985 mL, 10.57 mmol) was added at 0° C. and the reaction was stirred at RT for 1 h. The reaction progress was monitored by LCMS. After completion, the reaction was quenched with saturated bicarbonate solution (25 mL) and the mixture was extracted with DCM (3×25 mL). The combined organic layer was dried over sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain tert-butyl (3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)bicyclo[1.1.1]pentan-1-yl)carbamate (0.400 g, 28.1% yield). ¹H NMR (400 MHz, DMSO-d₆): δ 8.28 (br s, 1H), 8.19 (d, J=5.5 Hz, 11H), 7.78-7.61 (m, 1H), 7.38-7.32 (m, 4H), 7.30-7.22 (m, 3H), 7.11-7.04 (m, 1H), 6.66 (d, J=5.6 Hz, 1H), 4.72 (quin, J=6.7 Hz, 1H), 4.33-3.98 (m, 5H), 3.69 (s, 2H), 3.47 (s, 2H), 2.85-2.72 (m, 4H), 2.71-2.67 (m, 2H), 2.41 (br s, 2H), 2.27-2.16 (m, 3H), 1.99-1.83 (m, 4H), 1.41-1.06 (m, 15H). LCMS (Method B): Rt=1.64 min, m/z=790.0 [M+H]⁺, 97.75%.

### Intermediate 30. (S)-2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(pyrrolidin-3-yl)benzamide xHCl

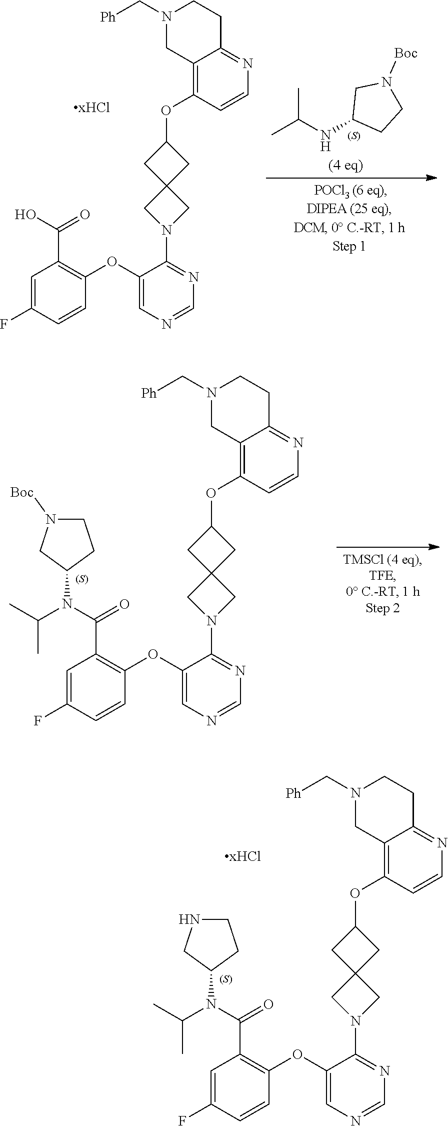

### Step 1. tert-Butyl (S)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)pyrrolidine-1-carboxylate

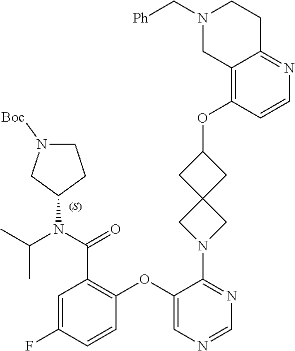

The reaction was performed following the same procedure in four batches (4×0.5 g).

To a stirred solution of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid (1.5 g, 2.64 mmol) in DCM (75 mL), tert-butyl (S)-3-(isopropylamino)pyrrolidine-1-carboxylate (2.414 g, 10.57 mmol) and DIPEA (8.54 g, 66.1 mmol) were added at RT. The reaction was stirred at RT for 5 min. POCl₃ (2.431 g, 15.86 mmol) was added at 0° C. and the reaction was stirred at RT for 1 h. The reaction progress was monitored by LCMS. After completion, the crude reaction mixtures from four batches were combined and quenched with aqueous NaHCO₃ solution (60 mL), and the mixture was extracted with DCM (2×50 mL). The combined organic layer was dried over sodium sulfate and filtered, and the filtrate was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN) to afford tert-butyl (S)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)pyrrolidine-1-carboxylate (1.1 g, 40.14% combined yield) as a solid. LCMS (Method A): Rt=2.26 min, m/z=778.4 [M+H]⁺, 98.47%.

### Step 2. (S)-2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(pyrrolidin-3-yl)benzamide xHCl

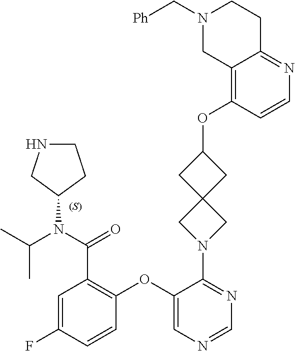

In a 25 mL round bottom flask, tert-butyl (S)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)pyrrolidine-1-carboxylate (0.75 g, 0.964 mmol) was dissolved in TFE (10 mL). To this solution, TMSCl (0.489 mL, 3.86 mmol) was added at 0° C. under a nitrogen atmosphere. The reaction was stirred at RT for 1 h. The reaction progress was monitored by TLC (10% MeOH in DCM). After completion, the reaction was concentrated under reduced pressure. The residue was re-concentrated from EtOAc (2×50 mL) to afford crude (S)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(pyrrolidin-3-yl)benzamide xHCl (0.65 g, 93% yield) as a solid. This material was used without further purification. LCMS (Method B): Rt=1.05 min, m/z=678.2 [M+H]⁺, 98.42%.

### Intermediate 31. (R)-2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(pyrrolidin-3-yl)benzamide xHCl

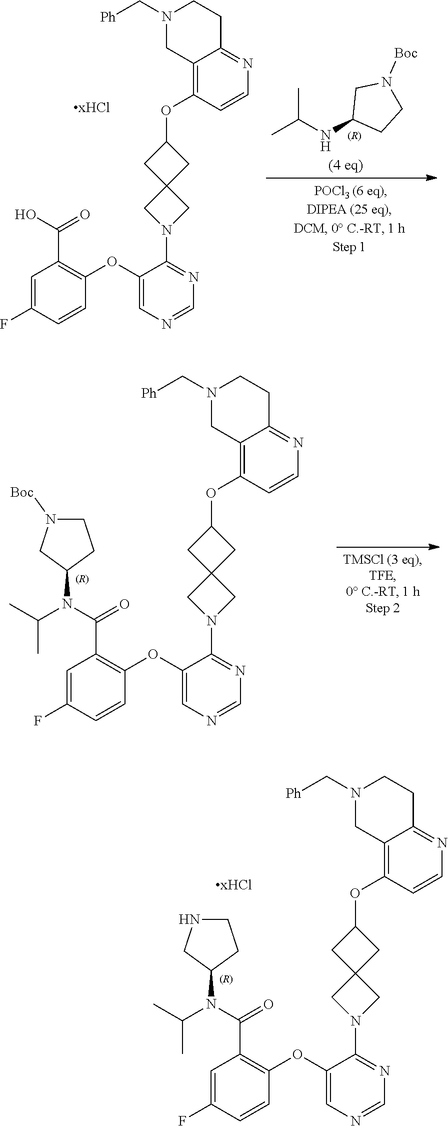

### Step 1. tert-Butyl (R)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)pyrrolidine-1-carboxylate

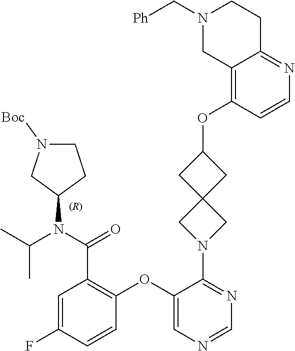

This compound was synthesized following the procedure described for the synthesis of Intermediate 30, Step 1, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid xHCl, except that 4 equivalents of tert-butyl (R)-3-(isopropylamino)pyrrolidine-1-carboxylate was used. The crude product was purified by reverse phase chromatography using a SiliCycle SiliaSep C18 column (40 g) on Biotage isolera one (Mobile phase A: 10 mM NH₄HCO₃ in water, B: ACN) to obtain tert-butyl (R)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)pyrrolidine-1-carboxylate as a solid.

Yield: 21.70%; LCMS (Method F): Rt=2.26 min, m/z=778.3 [M+H]⁺, 95.18%.

### Step 2. (R)-2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(pyrrolidin-3-yl)benzamide. xHCl

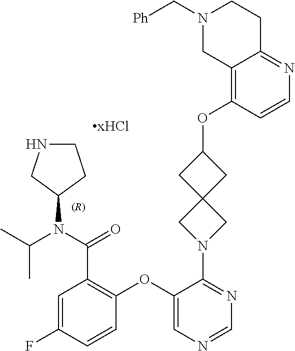

This compound was synthesized following the procedure described for the synthesis of Intermediate 30, Step 2, starting with 1 equivalent tert-butyl (R)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)pyrrolidine-1-carboxylate, except that 3 equivalents of TMSCl was used. The reaction was concentrated under reduced pressure to obtain crude (R)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(pyrrolidin-3-yl)benzamide xHCl. This material was used without further purification.

Yield: 64.8%; LCMS (Method A): Rt=2.05 min, m/z=678.3 [M+H]⁺, 70.88%.

### Intermediate 32. tert-Butyl 4-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)piperidine-1-carboxylate

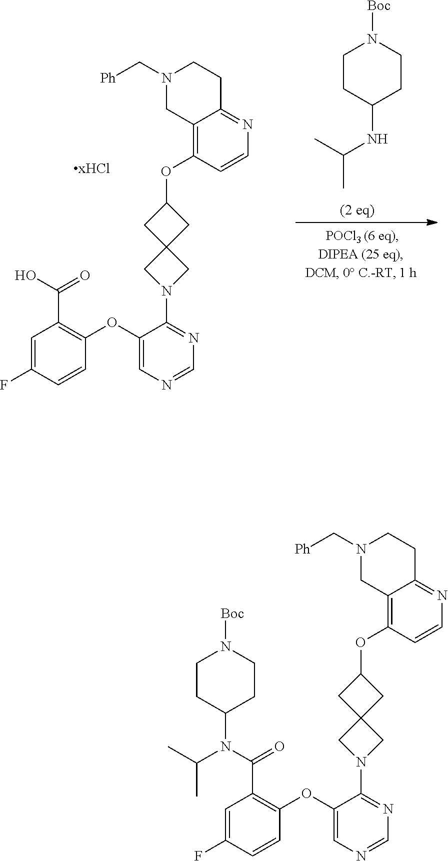 [SMILES: O=C(O)c1cc(F)ccc1Oc1cncnc1N1CC2(CC(Oc3ccnc4c3CN(Cc3ccccc3)CC4)C2)C1 ; CC(C)NC1CCN(C(=O)OC(C)(C)C)CC1 ; CC(C)N(C(=O)c1cc(F)ccc1Oc1cncnc1N1CC2(CC(Oc3ccnc4c3CN(Cc3ccccc3)CC4)C2)C1)C1CCN(C(=O)OC(C)(C)C)CC1]

This compound was synthesized following the procedure described for the synthesis Intermediate 29, Step 1, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid, except that 2 equivalents of tert-butyl 4-(isopropylamino)piperidine-1-carboxylate was used. The reaction was quenched with water and the mixture was extracted with DCM. The combined organic layer was washed with cold water and brine solution. The organic layer was dried over anhydrous sodium sulfate and filtered, and the filtrate was concentrated under reduced pressure. The crude compound was purified by reverse phase chromatography using a SiliCycle SiliaSep C18 column (120 g) on Biotage isolera one (Mobile phase A: 10 mM NH₄HCO₃ in water, B: ACN). The pure fractions were concentrated under reduced pressure to afford tert-butyl 4-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)piperidine-1-carboxylate as a solid.

Yield: 58.74% yield; LCMS (Method A): Rt=2.30 min, m/z=792.4 [M+H]⁺, 98.06%.

### Intermediate 33. (1s,3s)-3-(5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclobutyl 4-methylbenzenesulfonate

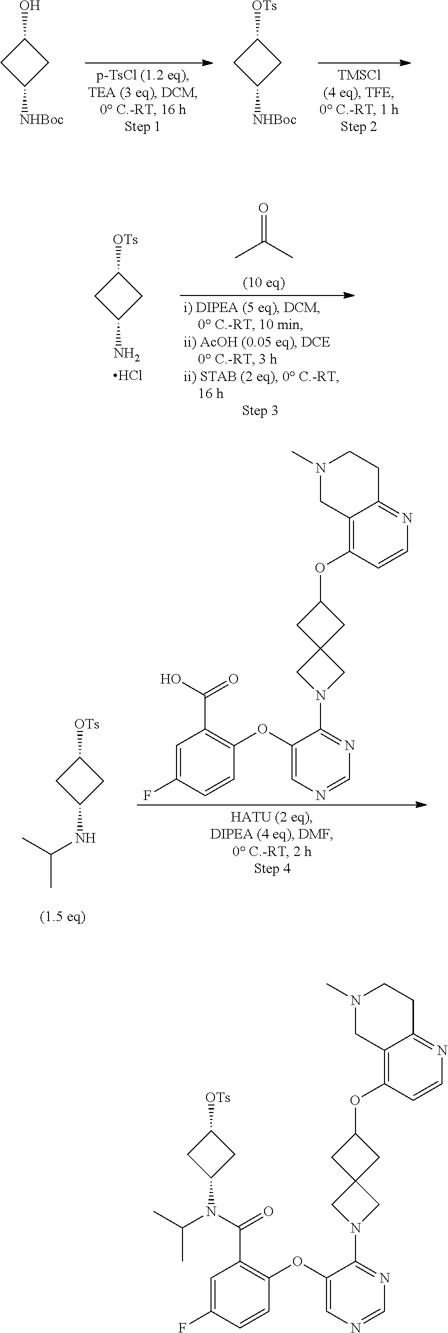

### Step 1. (1s,3s)-3-((tert-Butoxycarbonyl)amino)cyclobutyl 4-methylbenzenesulfonate

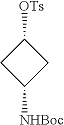 [SMILES: Cc1ccc(S(=O)(=O)O[C@H]2C[C@@H](NC(=O)OC(C)(C)C)C2)cc1]

To a stirred solution of tert-butyl ((1s,3s)-3-hydroxycyclobutyl)carbamate (2 g, 10.68 mmol) in DCM (10 mL), TEA (0.962 mL, 6.90 mmol) was added at 0° C. To this reaction mixture, p-TsCl (526 mg, 2.76 mmol) was added and the reaction was stirred at RT for 16 h. The reaction progress was monitored by LCMS. After completion, the reaction was cooled to 0° C. and quenched with saturated NaHCO₃ solution (25 mL), and the mixture was extracted with DCM (3×35 mL). The combined organic layer was washed with brine (50 mL), dried over sodium sulfate, and filtered. The filtrate was concentrated under reduced pressure. The crude was purified by silica gel flash column chromatography using EtOAc in hexane (product was eluted at 30% EtOAc in hexane) to obtain (1s,3s)-3-((tert-butoxycarbonyl)amino)cyclobutyl 4-methylbenzenesulfonate (2.5 g, 68.4% yield) as a solid. ¹H NMR (400 MHz, CDCl₃): δ 7.81-7.77 (m, 2H), 7.36 (d, J=8.0 Hz, 2H), 4.65 (brs, 1H), 4.51 (quin, J=7.2 Hz, 1H), 3.86-3.64 (m, 1H), 2.76-2.66 (m, 2H), 2.47 (s, 3H), 2.08-1.96 (m, 2H), 1.43 (s, 9H).

### Step 2. (1s,3s)-3-Aminocyclobutyl 4-methylbenzenesulfonate Hydrochloride

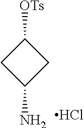 [SMILES: Cc1ccc(S(=O)(=O)O[C@H]2C[C@@H](N)C2)cc1 ; Cl]

To a stirred solution of (1s,3s)-3-((tert-butoxycarbonyl)amino)cyclobutyl 4-methylbenzenesulfonate (1.8 g, 5.27 mmol) in 2,2,2-trifluoroethanol (5 mL), TMSCl (2.67 mL, 21.09 mmol) was added at 0° C. The reaction was stirred at RT for 1 h. The reaction progress was monitored by TLC (40% EtOAc in hexane). After completion, the reaction was concentrated under reduced pressure. The residue was re-concentrated from EtOAc (2×10 mL) to obtain crude (1s,3s)-3-aminocyclobutyl 4-methylbenzenesulfonate hydrochloride (1.5 g, quantitative yield) as a solid. This material was used without further purification. LCMS (Method A): Rt=1.44 min, m/z=242.1 [M+H]⁺, 99.34%.

### Step 3. (1s,3s)-3-(Isopropylamino)cyclobutyl 4-methylbenzenesulfonate

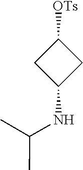 [SMILES: Cc1ccc(S(=O)(=O)O[C@H]2C[C@@H](NC(C)C)C2)cc1]

This compound was synthesized following the procedure described for the synthesis of Intermediate 1, starting with 1 equivalent of (1s,3s)-3-aminocyclobutyl 4-methylbenzenesulfonate hydrochloride, except that 5 equivalents of DIPEA and 10 equivalents of acetone were used, and DCE was used as the solvent. After work-up, the filtrate was concentrated under reduced pressure to obtain crude (1s,3s)-3-(isopropylamino)cyclobutyl 4-methylbenzenesulfonate as a semi-solid. This material was used without further purification.

Yield: 58.8%; ¹H NMR (400 MHz, CDCl₃): δ 7.82-7.78 (m, 2H), 7.35 (d, J=8.0 Hz, 2H), 4.55 (quin, J=7.4 Hz, 1H), 2.95-2.86 (m, 1H), 2.84-2.74 (m, 1H), 2.69-2.60 (m, 2H), 2.47 (s, 3H), 1.93-1.82 (m, 2H), 1.01 (d, J=6.3 Hz, 6H), one proton merged with solvent peak; LCMS (Method A): Rt=1.75 min, m/z=284.1 [M+H]⁺, 69.32%.

### Step 4. (1s,3s)-3-(5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclobutyl 4-methylbenzenesulfonate

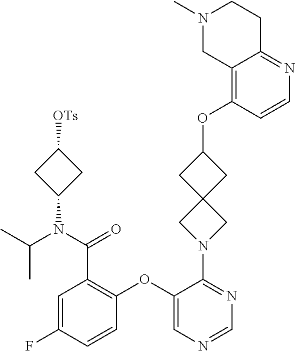 [SMILES: Cc1ccc(S(=O)(=O)O[C@H]2C[C@@H](N(C(=O)c3cc(F)ccc3Oc3cncnc3N3CC4(CC(Oc5ccnc6c5CN(C)CC6)C4)C3)C(C)C)C2)cc1]

To a stirred solution of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid (1 g, 2.034 mmol) and (1s,3s)-3-(isopropylamino)cyclobutyl 4-methylbenzenesulfonate (0.865 g, 3.05 mmol) in DMF (10 mL), DIPEA (1.421 mL, 8.14 mmol) and HATU (1.547 g, 4.07 mmol) were added at 0° C. The reaction was stirred at RT for 2 h. The reaction progress was monitored by LCMS. After completion, the reaction was diluted with cold water (25 mL), and the mixture was extracted with EtOAc (3×40 mL). The combined organic layer was washed with brine (50 mL), dried over anhydrous sodium sulfate and filtered, and the filtrate was concentrated under reduced pressure. The crude compound was purified by reverse phase chromatography using a SiliCycle SiliaSep C18 column (120 g) on Biotage isolera one (Mobile phase A: 10 mM NH₄HCO₃ in water, B: ACN) to obtain (1s,3s)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclobutyl 4-methylbenzenesulfonate (1.1 g, 70.7% yield) as a solid. ¹H NMR (400 MHz, DMSO-d₆): δ 8.31-8.25 (m, 1H), 8.18 (d, J=5.6 Hz, 1H), 7.79-7.72 (m, 3H), 7.45 (d, J=7.8 Hz, 2H), 7.32-7.21 (m, 2H), 7.15-7.00 (m, 1H), 6.69-6.65 (m, 1H), 4.79-4.69 (m, 1H), 4.61-4.41 (m, 1H), 4.30-4.03 (m, 4H), 3.81-3.52 (m, 2H), 3.37 (br s, 2H), 3.28-3.18 (m, 1H), 2.86-2.72 (m, 4H), 2.69-2.60 (m, 2H), 2.40 (s, 3H), 2.36 (s, 3H), 2.34-2.18 (m, 5H), 1.40-0.91 (m, 6H); LCMS (Method A): Rt=2.00 min, m/z=757.1 [M+H]⁺, 98.94%; Chiral SFC (Method AF: Rt=3.99 min, 100%.

### Intermediate 34. 6-Benzyl-4-((2-(3,6-dichloro-1,2,4-triazin-5-yl)-2-azaspiro[3.3]heptan-6-yl)oxy)-5,6,7,8-tetrahydro-1,6-naphthyridine

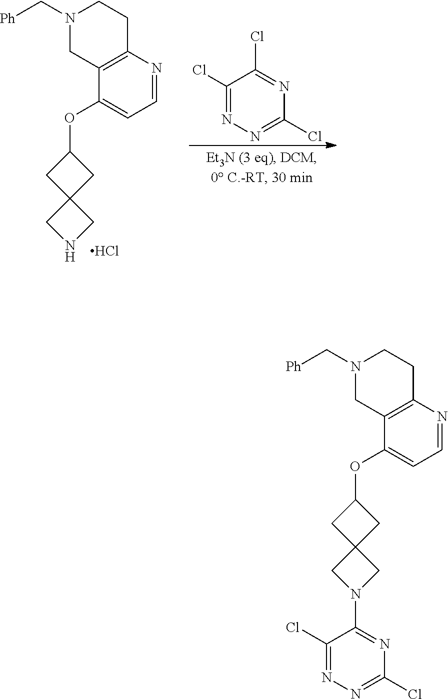

To a stirred solution of 4-((2-azaspiro[3.3]heptan-6-yl)oxy)-6-benzyl-5,6,7,8-tetrahydro-1,6-naphthyridine hydrochloride (49 g, 132 mmol) in DCM (490 mL), Et₃N (55.6 mL, 395 mmol) was added at 0° C. under a nitrogen atmosphere. To this reaction mixture, 3,5,6-trichloro-1,2,4-triazine (24.30 g, 132 mmol) was added, and the reaction was stirred at RT for 30 min. The progress of the reaction was monitored by TLC (5% MeOH in DCM). After completion, the reaction was quenched with cold water (100 mL) and extracted with DCM (3×150 mL). The combined organic layer was washed with brine solution (200 mL), dried over anhydrous sodium sulfate and filtered, and the filtrate was concentrated under reduced pressure. The residue was triturated with 50% EtOAc in hexane (3×50 mL), and the organic layer was decanted. The resulting solid was dried under vacuum to obtain 6-benzyl-4-((2-(3,6-dichloro-1,2,4-triazin-5-yl)-2-azaspiro[3.3]heptan-6-yl)oxy)-5,6,7,8-tetrahydro-1,6-naphthyridine (54 g, 85% yield) as a solid. ¹H NMR (400 MHz, DMSO-d₆): δ 8.22 (d, J=5.6 Hz, 1H), 7.40-7.25 (m, 5H), 6.68 (d, J=5.6 Hz, 1H), 4.81-4.67 (m, 3H), 4.25-4.12 (m, 2H), 3.71 (br s, 2H), 3.47 (br s, 2H), 2.85-2.77 (m, 4H), 2.72 (br s, 2H), 2.32-2.23 (m, 2H); LCMS (Method G): Rt=2.08 min, m/z=483.2 [M+H]⁺, 99.84%.

### Intermediate 35. N-((1r,3r)-3-(Dimethylamino)-3-methylcyclobutyl)-5-fluoro-2-hydroxy-N-isopropylbenzamide

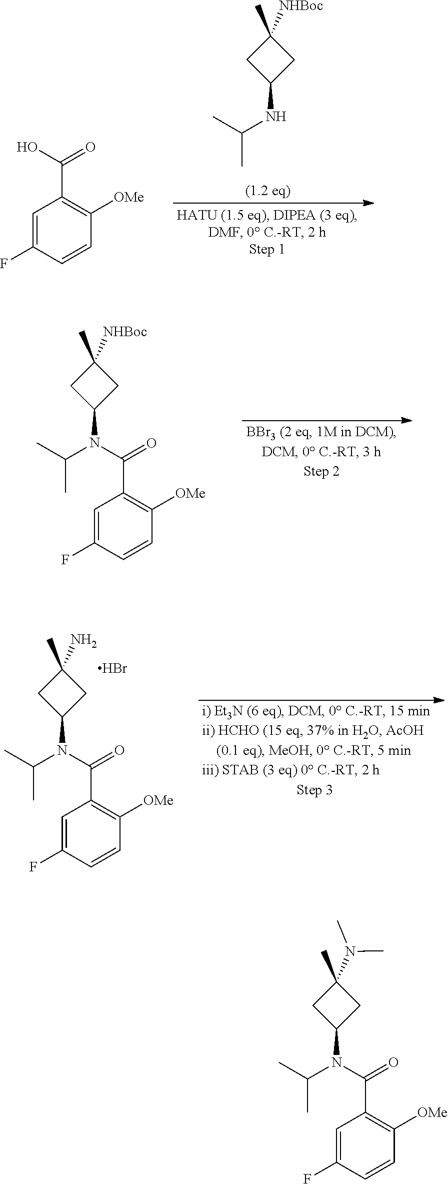 [SMILES: COc1ccc(F)cc1C(=O)O ; CC(C)N[C@H]1C[C@](C)(NC(=O)OC(C)(C)C)C1 ; COc1ccc(F)cc1C(=O)N(C(C)C)[C@H]1C[C@](C)(NC(=O)OC(C)(C)C)C1 ; COc1ccc(F)cc1C(=O)N(C(C)C)[C@H]1C[C@](C)(N)C1 ; Br ; COc1ccc(F)cc1C(=O)N(C(C)C)[C@H]1C[C@](C)(N(C)C)C1]

### Step 1. tert-Butyl ((1r,3r)-3-(5-fluoro-N-isopropyl-2-methoxybenzamido)-1-methylcyclobutyl)carbamate

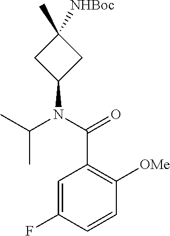 [SMILES: COc1ccc(F)cc1C(=O)N(C(C)C)[C@H]1C[C@](C)(NC(=O)OC(C)(C)C)C1]

In a 50 mL round bottom flask under a nitrogen atmosphere, 5-fluoro-2-methoxybenzoic acid (300 mg, 1.763 mmol) and tert-butyl ((1r,3r)-3-(isopropylamino)-1-methylcyclobutyl)carbamate (513 mg, 2.116 mmol) were dissolved in DMF (5 mL). To this reaction mixture, HATU (1006 mg, 2.64 mmol) and DIPEA (0.921 mL, 5.29 mmol) were added at 0° C. and the reaction was stirred at RT for 2 h. The reaction progress was monitored by TLC (10% MeOH in DCM). After completion, the reaction was quenched with water (20 mL) and the mixture was extracted with EtOAc (2×25 mL). The combined organic layer was dried over anhydrous sodium sulfate and filtered. The filtrate was concentrated under reduced pressure to obtain crude tert-butyl ((1r,3r)-3-(5-fluoro-N-isopropyl-2-methoxybenzamido)-1-methylcyclobutyl)carbamate (400 mg, 25.3% yield) as a solid. This material was used without further purification. LCMS (Method A): Rt=2.10 min, m/z=395.2 [M+H]⁺, 88.99%.

### Step 2. N-((1r,3r)-3-Amino-3-methylcyclobutyl)-5-fluoro-2-hydroxy-N-isopropylbenzamide Hydrobromide

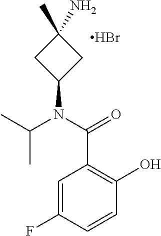 [SMILES: CC(C)N(C(=O)c1cc(F)ccc1O)[C@H]1C[C@](C)(N)C1 ; Br]

To a stirred solution of tert-butyl ((1r,3r)-3-(5-fluoro-N-isopropyl-2-methoxybenzamido)-1-methylcyclobutyl)carbamate (600 mg, 1.521 mmol) in DCM (3 mL), BBr₃ (3.04 mL, 3.04 mmol, 1 M in DCM) was added at 0° C. The reaction was stirred at RT for 3 h under a nitrogen atmosphere. The reaction progress was monitored by TLC (10% MeOH in DCM). After completion, the reaction was quenched with MeOH (25 mL) at 0° C. and the mixture was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain N-((1r,3r)-3-amino-3-methylcyclobutyl)-5-fluoro-2-hydroxy-N-isopropylbenzamide hydrobromide (420 mg, 72.0% yield) as a solid. ¹H NMR (400 MHz, DMSO-d₆): δ 9.64 (m, 1H), 8.15 (br s, 3H), 7.03 (dt, J=3.1, 8.7 Hz, 1H), 6.92 (dd, J=3.0, 8.5 Hz, 1H), 6.86 (dd, J=4.5, 8.9 Hz, 1H), 4.37 (quin, J=8.8 Hz, 1H), 3.69-3.52 (m, 11H), 3.15-3.01 (m, 2H), 2.38-2.25 (m, 2H), 1.49 (s, 3H), 1.25-0.98 (m, 6H); LCMS (Method A): Rt=1.31 min, m/z=281.1 [M+H]⁺, 68.87%.

### Step 3. N-((1r,3r)-3-(dimethylamino)-3-methylcyclobutyl)-5-fluoro-2-hydroxy-N-isopropylbenzamide

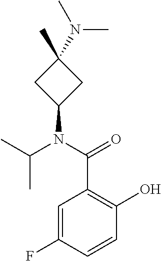 [SMILES: CC(C)N(C(=O)c1cc(F)ccc1O)[C@H]1C[C@](C)(N(C)C)C1]

To a stirred solution of N-((1r,3r)-3-amino-3-methylcyclobutyl)-5-fluoro-2-hydroxy-N-isopropylbenzamide (420 mg, 1.498 mmol) in DCM (5 mL), TEA (1.253 mL, 8.99 mmol) was added, and the reaction was stirred at RT for 15 min. The solvent was concentrated under reduced pressure, and the residue was dissolved in MeOH (5 mL). To this mixture, formaldehyde (1824 mg, 22.47 mmol, 37% in H₂O) and AcOH (8.58 μL, 0.150 mmol) were added at 0° C., and the reaction was stirred at RT for 5 min. STAB (953 mg, 4.49 mmol) was added at 0° C., and the reaction was stirred RT for 2 h. The reaction progress was monitored by TLC (10% MeOH in DCM). After completion, the reaction was concentrated under reduced pressure. Aqueous Na₂CO₃ (10 mL) solution was added to the residue and the mixture was extracted with 10% MeOH in DCM (2×75 mL). The combined organic layer was washed with brine solution (10 mL), dried over anhydrous sodium sulfate, and filtered. The filtrate was concentrated under reduced pressure to obtain crude N-((1r,3r)-3-amino-3-methylcyclobutyl)-5-fluoro-2-hydroxy-N-isopropylbenzamide (380 mg, 82% yield) as a solid. This material was used without further purification. LCMS (Method A): Rt=1.48 min, m/z=309.0 [M+H]⁺, 99.20%.

### Intermediate 36. (±)-5-Fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoic Acid

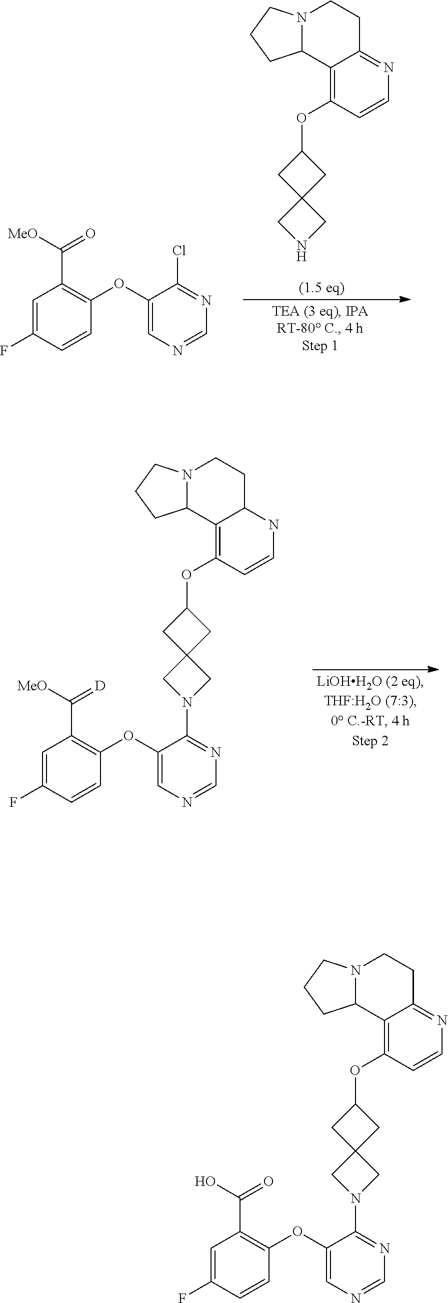

### Step 1. (±)-Methyl 5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)benzoate

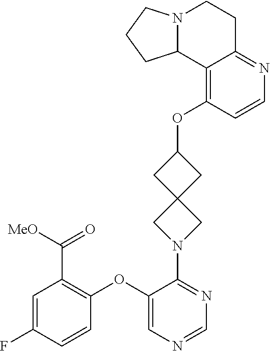 [SMILES: COC(=O)c1cc(F)ccc1Oc1cncnc1N1CC2(CC(Oc3ccnc4c3C3CCCN3CC4)C2)C1]

To a stirred solution of (±)-1-((2-azaspiro[3.3]heptan-6-yl)oxy)-5,6,8,9,10,10a-hexahydropyrrolo[2,1-f][1,6]naphthyridine (0.854 g, 2.65 mmol) in IPA (15 mL), TEA (0.746 mL, 5.31 mmol) and methyl 2-((4-chloropyrimidin-5-yl)oxy)-5-fluorobenzoate (0.5 g, 1.769 mmol) were added at RT. The reaction was stirred at 80° C. for 4 h under a nitrogen atmosphere. The reaction progress was monitored by LCMS and TLC (20% MeOH in DCM). After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain (±)-methyl 5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoate (310 mg, 21.76%) as a solid. LCMS (Method A): Rt=1.78-1.87 min, m/z=532.3 [M+H]⁺, 66.28%.

Note: (±)-5-Fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoic acid (170 mg, 9.85% yield) was also obtained. LCMS (Method A): Rt=1.20 min, m/z=518.0 [M+H]⁺, 53.06%.

### Step 2. (±)-5-Fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)benzoic Acid

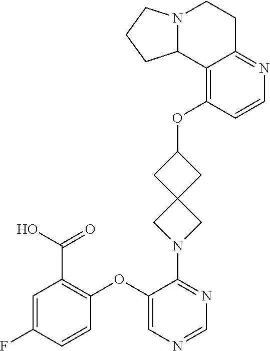 [SMILES: O=C(O)c1cc(F)ccc1Oc1cncnc1N1CC2(CC(Oc3ccnc4c3C3CCCN3CC4)C2)C1]

To a stirred solution of (±)-methyl 5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoate (0.41 g, 0.771 mmol) in a mixture of THF:H₂O (2.33:1, 10 mL), LiOH·H₂O (0.065 mg, 1.543 mmol) was added at 0° C. The reaction was stirred at RT for 4 h. The reaction progress was monitored by TLC (10% MeOH in DCM). After completion, the reaction was concentrated under reduced pressure. The crude was diluted with water (5 mL) and acidified with 1.5N HCl (to pH˜6), and the mixture was concentrated under reduced pressure. The crude was purified reverse phase chromatography using a SiliCycle SiliaSep C18 column (40 g) on Biotage isolera one (Mobile phase A: 10 mM NH₄HCO₃ in water, B: ACN) to obtain (±)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoic acid (430 mg, quantitative yield) as a solid. LCMS (Method A): Rt=1.20 min, m/z=518.1 [M+H]⁺, 97.72%.

### Intermediate 37. Benzyl ((1r,3r)-3-amino-3-methylcyclobutyl)(isopropyl)carbamate

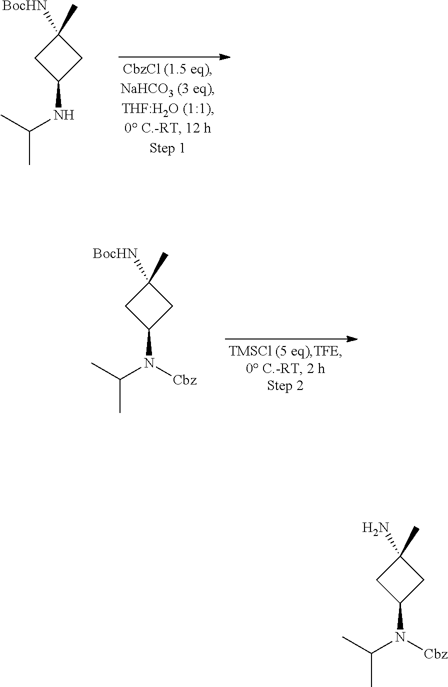 [SMILES: CC(C)N[C@H]1C[C@](C)(NC(=O)OC(C)(C)C)C1 ; CC(C)N(C(=O)OCc1ccccc1)[C@H]1C[C@](C)(NC(=O)OC(C)(C)C)C1 ; CC(C)N(C(=O)OCc1ccccc1)[C@H]1C[C@](C)(N)C1]

### Step 1. Benzyl ((1r,3r)-3-((tert-butoxycarbonyl)amino)-3-methylcyclobutyl)(isopropyl)carbamate

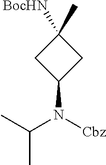 [SMILES: CC(C)N(C(=O)OCc1ccccc1)[C@H]1C[C@](C)(NC(=O)OC(C)(C)C)C1]

To a stirred solution of tert-butyl ((1r,3r)-3-(isopropylamino)-1-methylcyclobutyl)carbamate (2.1 g, 8.66 mmol) in THF:H₂O (1:1, 54 mL), sodium bicarbonate (2.184 g, 26.0 mmol) and Cbz-Cl (1.855 mL, 13.00 mmol) were added at 0° C. The reaction was stirred at RT for 12 h. The reaction progress was monitored by TLC (30% EtOAc in hexane). After completion, the reaction was quenched with ice-cold water (25 mL), and the mixture was extracted with DCM (2×50 mL). The combined organic layer was dried over anhydrous sodium sulfate and filtered, and the filtrate was concentrated under reduced pressure. The crude was purified by silica gel flash column chromatography using EtOAc in hexane (product was eluted at 11% EtOAc in hexane) to obtain benzyl ((1r,3r)-3-((tert-butoxycarbonyl)amino)-3-methylcyclobutyl)(isopropyl)carbamate (2.5 g, 64.9% yield) as a liquid. ¹H NMR (400 MHz, CDCl₃): δ 7.44-7.30 (m, 5H), 5.15 (s, 2H), 4.62 (br s, 1H), 4.22-4.14 (m, 1H), 2.64-2.46 (m, 2H), 2.42-2.21 (m, 2H), 1.46 (s, 9H), 1.39-1.30 (br s, 3H), 1.17 (d, J=6.9 Hz, 6H), one proton merged with solvent peaks; LCMS (Method A): Rt=2.42 min, m/z=377.2 [M+H]⁺, 84.74%.

### Step 2. Benzyl ((1r,3r)-3-amino-3-methylcyclobutyl)(isopropyl)carbamate

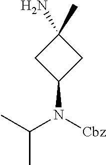 [SMILES: CC(C)N(C(=O)OCc1ccccc1)[C@H]1C[C@](C)(N)C1]

To a stirred solution of benzyl ((1r,3r)-3-((tert-butoxycarbonyl)amino)-3-methylcyclobutyl)isopropyl)carbamate (2.5 g, 6.64 mmol) in TFE (12 mL), TMSCl (4.21 mL, 33.1 mmol) was added at 0° C., and the reaction was stirred at RT for 2 h. The reaction progress was monitored by TLC (10% MeOH in DCM). After completion, the reaction was concentrated under reduced pressure to obtain benzyl ((1r,3r)-3-amino-3-methylcyclobutyl)(isopropyl)carbamate hydrochloride. The residue was basified using aqueous NaOH solution (20 mL) and the mixture was extracted with DCM (2×50 mL). The combined organic layer was washed with brine solution, dried over anhydrous sodium sulfate, and filtered. The filtrate was concentrated under reduced pressure to obtain benzyl ((1r,3r)-3-amino-3-methylcyclobutyl)(isopropyl)carbamate (1.6 g, 87% yield) as a liquid. ¹H NMR (400 MHz, DMSO-d₆): δ 7.43-7.31 (m, 5H), 5.07 (s, 2H), 4.28 (quin, J=8.8 Hz, 1H), 4.00 (spt, J=6.7 Hz, 1H), 2.33-2.24 (m, 2H), 1.89-1.79 (m, 2H), 1.78-1.61 (m, 2H), 1.12 (d, J=6.8 Hz, 6H), 1.08 (br s, 3H).

### Intermediate 38. (1r,3r)—N³-Isopropyl-N¹,N¹-trimethylcyclobutane-1,3-diamine

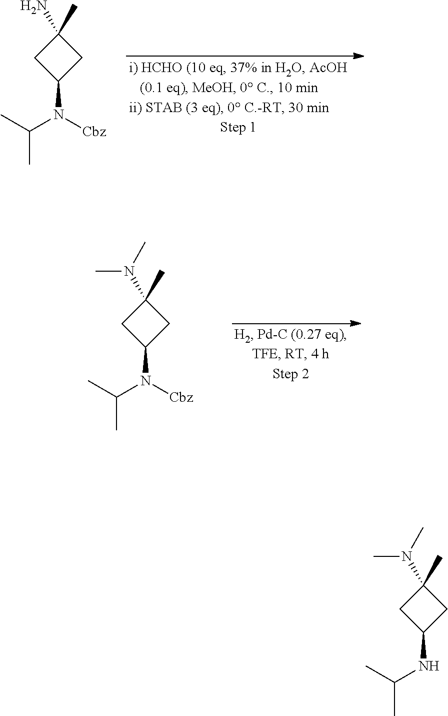 [SMILES: CC(C)N(C(=O)OCc1ccccc1)[C@H]1C[C@](C)(N)C1 ; CC(C)N(C(=O)OCc1ccccc1)[C@H]1C[C@](C)(N(C)C)C1 ; CC(C)N[C@H]1C[C@](C)(N(C)C)C1]

### Step 1. Benzyl ((1r,3r)-3-(dimethylamino)-3-methylcyclobutyl)(isopropyl)carbamate

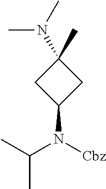 [SMILES: CC(C)N(C(=O)OCc1ccccc1)[C@H]1C[C@](C)(N(C)C)C1]

To a stirred solution of benzyl ((1r,3r)-3-amino-3-methylcyclobutyl)(isopropyl)carbamate (1 g, 3.62 mmol) in MeOH (10 mL), formaldehyde (2.69 mL, 36.2 mmol, 37% in H₂O) and AcOH (0.021 mL, 0.362 mmol) were added at 0° C., and the reaction was stirred at the same temperature for 10 min. To this reaction mixture, STAB (2.301 g, 10.85 mmol) was added at 0° C., and the reaction was stirred at RT for 30 min. The reaction progress was monitored by TLC (10% MeOH in DCM). After completion, the reaction was concentrated under reduced pressure. Aqueous NaHCO₃ (10 mL) was added to the residue and the mixture was extracted with EtOAc (2×30 mL). The combined organic layer was dried over anhydrous sodium sulfate and filtered, and the filtrate was concentrated under reduced pressure. The crude compound was purified by reverse phase chromatography using a SiliCycle SiliaSep C18 column (80 g) on Biotage isolera one (Mobile phase A: 10 mM NH₄HCO₃ in water, B: ACN) to obtain benzyl ((1r,3r)-3-(dimethylamino)-3-methylcyclobutyl)(isopropyl)carbamate (750 mg, 62.0% yield) as a solid. ¹H NMR (400 MHz, DMSO-d₆): δ 7.45-7.28 (m, 5H), 5.08 (s, 2H), 4.10-3.95 (m, 2H), 2.18-2.03 (m, 10H), 1.11 (d, J=6.8 Hz, 6H), 0.92 (br s, 3H); LCMS (Method A): Rt=2.02 min, m/z=305.1 [M+H]⁺, 91.03%.

### Step 2. (1r,3r)—N³-Isopropyl-N¹,N¹-trimethylcyclobutane-1,3-diamine

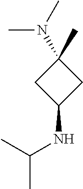

In a 50 mL round bottom flask under a nitrogen atmosphere, benzyl ((1r,3r)-3-(dimethylamino)-3-methylcyclobutyl)(isopropyl)carbamate (750 mg, 2.464 mmol) was dissolved 5 in TFE (10 mL). To this solution, Pd—C (750 mg, 0.705 mmol) was added, and the reaction was stirred at RT for 4 h under a hydrogen atmosphere (balloon pressure). The reaction progress was monitored by TLC (10% MeOH in DCM). After completion, the reaction was filtered through a Celite® pad, and the pad was washed with TFE (30 mL). The filtrate was concentrated under reduced pressure to obtain crude (1r,3r)—N³-isopropyl-N¹,N¹,1-trimethylcyclobutane-1,3-diamine (430 mg, quantitative yield) as a liquid. This material was used without further purification. ¹H NMR (400 MHz, DMSO-d₆): δ 3.19 (quin, J=7.3 Hz, 1H), 2.73 (spt, J=6.3 Hz, 1H), 2.29-2.18 (m, 2H), 2.06 (s, 6H), 1.48-1.40 (m, 2H), 1.02 (s, 3H), 0.95 (d, J=6.3 Hz, 6H), one proton merged with solvent peaks.

### Intermediate 39. (1r,3r)-3-(Azetidin-1-yl)-N-isopropyl-3-methylcyclobutan-1-amine

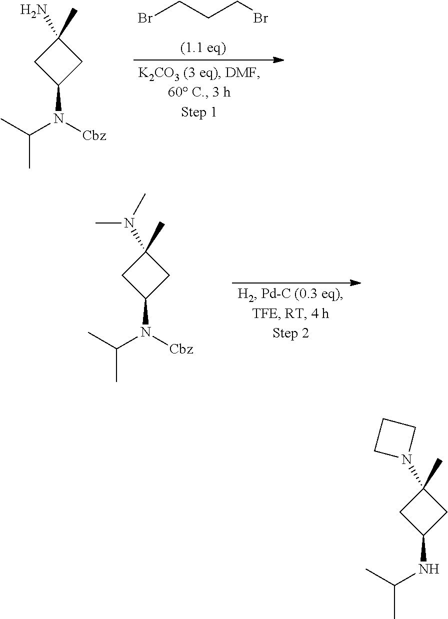 [SMILES: CC(C)N(C(=O)OCc1ccccc1)[C@H]1C[C@](C)(N)C1 ; BrCCCBr ; CC(C)N(C(=O)OCc1ccccc1)[C@H]1C[C@](C)(N(C)C)C1 ; CC(C)N[C@H]1C[C@](C)(N2CCC2)C1]

### Step 1. Benzyl ((1r,3r)-3-(azetidin-1-yl)-3-methylcyclobutyl)(isopropyl)carbamate

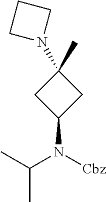 [SMILES: CC(C)N(C(=O)OCc1ccccc1)[C@H]1C[C@](C)(N2CCC2)C1]

In a 250 mL round bottom flask, benzyl ((1r,3r)-3-amino-3-methylcyclobutyl)(isopropyl)carbamate (1 g, 3.62 mmol) was dissolved in DMF (100 mL). To this solution, K₂CO₃ (1.5 g, 10.85 mmol) and 1,3-dibromopropane (0.406 mL, 3.98 mmol) were added at RT. The reaction was stirred at 60° C. for 3 h. The reaction progress was monitored by TLC (10% MeOH in DCM). After completion, the reaction was quenched with ice-cold water (50 mL) and the mixture was extracted with EtOAc (3×50 mL). The combined organic layer was washed with ice-cold brine (3×50 mL), dried over anhydrous sodium sulfate, and filtered, and the filtrate was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure to afford benzyl ((1r,3r)-3-(azetidin-1-yl)-3-methylcyclobutyl)(isopropyl)carbamate (800 mg, 69.5% yield) as a gummy solid. ¹H NMR (400 MHz, DMSO-d₆): δ 7.42-7.29 (m, 5H), 5.08 (s, 2H), 4.09-3.92 (m, 2H), 3.08 (t, J=6.9 Hz, 3H), 2.11 (br d, J=9.0 Hz, 3H), 1.86 (quin, J=6.9 Hz, 2H), 1.11 (d, J=6.8 Hz, 6H), 0.97 (br s, 3H), two protons merged with solvent peaks; LCMS (Method H): Rt=1.58 min, m/z=317.2 [M+H]⁺, 99.43%.

### Step 2. (1r,3r)-3-(Azetidin-1-yl)-N-isopropyl-3-methylcyclobutan-1-amine

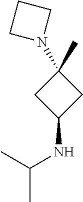

This compound was synthesized following the procedure described for the synthesis of Intermediate 38, step 2, starting with 1 equivalent of benzyl ((1r,3r)-3-(azetidin-1-yl)-3-methylcyclobutyl)(isopropyl)carbamate. After filtration, the combined filtrate was concentrated under reduced pressure to afford crude (1r,3r)-3-(azetidin-1-yl)-N-isopropyl-3-methylcyclobutan-1-amine as a semisolid. This material was used without further purification.

Yield: 56.5%. ¹H NMR (400 MHz, DMSO-d₆): δ 3.20-3.13 (m, 1H), 3.06 (t, J=6.9 Hz, 4H), 2.75-2.68 (m, 1H), 2.29-2.20 (m, 2H), 1.85 (quin, J=6.9 Hz, 2H), 1.43-1.36 (m, 2H), 1.04 (s, 3H), 0.94 (d, J=6.3 Hz, 6H), one proton merged with solvent peaks; LCMS (Method A): Rt=1.03 min, m/z=183.1 [M+H]⁺, 49.43%.

### Intermediate 40. (1r,3r)—N-Isopropyl-3-methyl-3-(pyrrolidin-1-yl)cyclobutan-1-amine (Procedure 1)

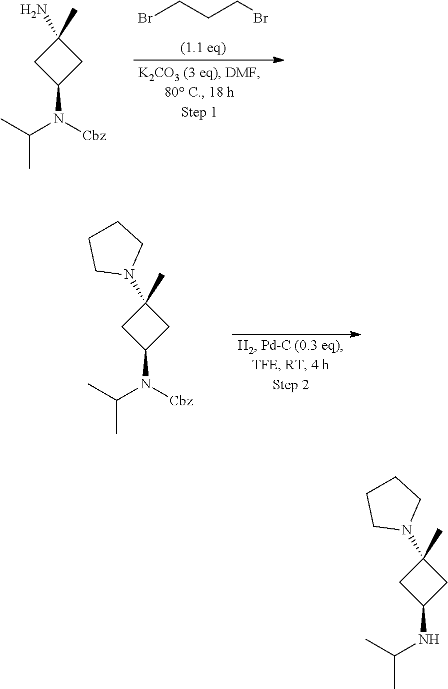 [SMILES: CC(C)N(C(=O)OCc1ccccc1)[C@H]1C[C@](C)(N)C1 ; BrCCCBr ; CC(C)N(C(=O)OCc1ccccc1)[C@H]1C[C@](C)(N2CCCC2)C1 ; CC(C)N[C@H]1C[C@](C)(N2CCCC2)C1]

### Step 1. Benzyl isopropyl((1r,3r)-3-methyl-3-(pyrrolidin-1-yl)cyclobutyl)carbamate

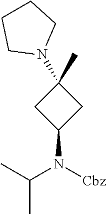 [SMILES: CC(C)N(C(=O)OCc1ccccc1)[C@H]1C[C@](C)(N2CCCC2)C1]

In a 40 mL pressure vial, benzyl ((1r,3r)-3-amino-3-methylcyclobutyl)(isopropyl)carbamate (1.0 g, 3.62 mmol) was dissolved in ACN (15 mL). To this solution, K₂CO₃ (1.500 g, 10.85 mmol) and 1,4-dibromobutane (0.859 g, 3.98 mmol) were added at RT, and the reaction was stirred at 80° C. for 18 h. The reaction progress was monitored by LCMS. After completion, the reaction was quenched with 10% NaHCO₃ solution (15 mL) and the mixture was extracted with EtOAc (3×45 mL). The combined organic layer was washed with brine solution (50 mL), dried over anhydrous sodium sulfate, and filtered. The filtrate was concentrated under reduced pressure to obtain crude benzyl isopropyl((1r,3r)-3-methyl-3-(pyrrolidin-1-yl)cyclobutyl)carbamate (1.35 g, quantitative yield) as a liquid. This material was used without further purification. ¹H NMR (400 MHz, DMSO-d₆): δ 7.44-7.30 (m, 5H), 5.08 (s, 2H), 4.16-3.92 (m, 2H), 2.26-2.06 (m, 4H), 1.72-1.62 (m, 4H), 1.12 (d, J=6.8 Hz, 6H), 1.03 (br s, 3H), four protons merged with solvent peaks; LCMS (Method H): Rt=1.59 min, m/z=331.2 [M+H]⁺, 97.62%.

### Step 2. (1r,3r)—N-Isopropyl-3-methyl-3-(pyrrolidin-1-yl)cyclobutan-1-amine

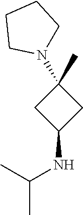

This compound was synthesized following the procedure described for the synthesis of Intermediate 38, step 2, starting with 1 equivalent of benzyl isopropyl((1r,3r)-3-methyl-3-(pyrrolidin-1-yl)cyclobutyl)carbamate. After filtration, the filtrate was concentrated under reduced pressure to obtain crude (1r,3r)—N-isopropyl-3-methyl-3-(pyrrolidin-1-yl)cyclobutan-1-amine as a gummy liquid. This material was used without further purification.

Yield: 97%; ¹H NMR (400 MHz, DMSO-d₆): δ 3.51-3.43 (m, 1H), 3.01-2.92 (m, 1H), 2.58-2.52 (m, 4H), 2.45-2.35 (m, 2H), 1.80-1.64 (m, 6H), 1.16 (s, 3H), 1.06 (d, J=6.4 Hz, 6H), one proton merged with solvent peaks.

### Intermediate 40. (1r,3r)—N-isopropyl-3-methyl-3-(pyrrolidin-1-yl)cyclobutan-1-amine (Procedure 2)

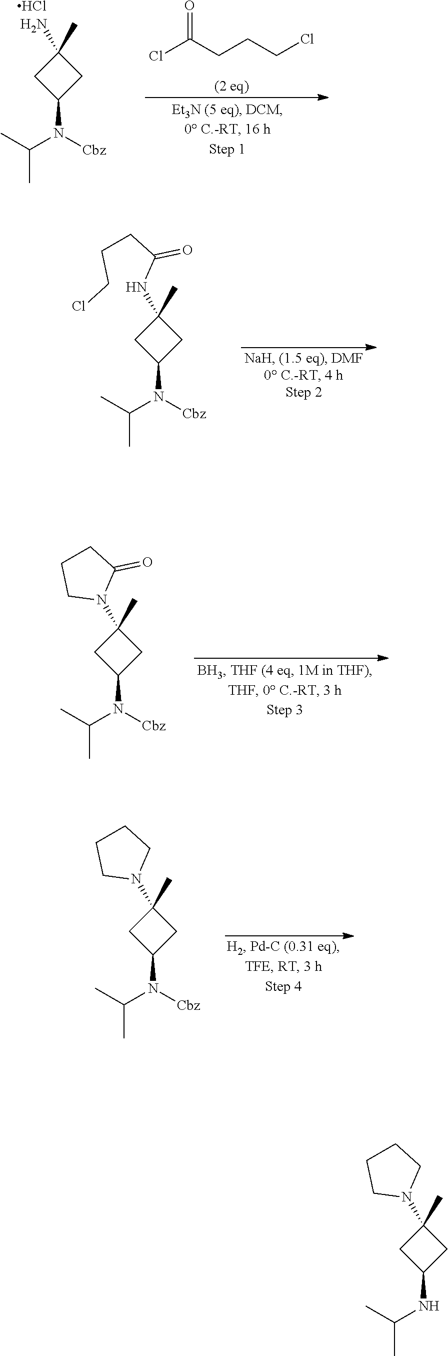

### Step 1. Benzyl ((1r,3r)-3-(4-chlorobutanamido)-3-methylcyclobutyl)(isopropyl)carbamate

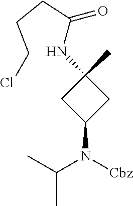 [SMILES: CC(C)N(C(=O)OCc1ccccc1)[C@H]1C[C@](C)(NC(=O)CCCCl)C1]

In a dried, 50 mL round bottom flask under a nitrogen atmosphere, benzyl ((1r,3r)-3-amino-3-methylcyclobutyl)(isopropyl)carbamate hydrochloride (580 mg, 1.854 mmol) was dissolved in DCM (7 mL). To this solution, TEA (1.292 mL, 9.27 mmol) and 4-chlorobutanoyl chloride (523 mg, 3.71 mmol) were added at 0° C., and the reaction was stirred at RT for 16 h. The reaction progress was monitored by TLC (10% MeOH in DCM). After completion, the reaction was cooled to 0° C.-5° C. and quenched with NaHCO₃ solution (15 mL), and the mixture was extracted with EtOAc (3×40 mL). The combined organic layers were washed with brine solution (25 mL), dried over anhydrous sodium sulfate and filtered, and the filtrate was concentrated under reduced pressure to afford crude benzyl ((1r,3r)-3-(4-chlorobutanamido)-3-methylcyclobutyl)(isopropyl)carbamate (600 mg, 74.3% yield) as a gummy compound. This material was used without further purification. LCMS (Method A): Rt=2.07 min, m/z=381.1 [M+H]⁺, 87.47%.

### Step 2. Benzyl isopropyl((1r,3r)-3-methyl-3-(2-oxopyrrolidin-1-yl)cyclobutyl)carbamate

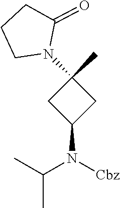 [SMILES: CC(C)N(C(=O)OCc1ccccc1)[C@H]1C[C@](C)(N2CCCC2=O)C1]

In a dried, 25 mL two neck round bottom flask under a nitrogen atmosphere, benzyl ((1r,3r)-3-(4-chlorobutanamido)-3-methylcyclobutyl)(isopropyl)carbamate (600 mg, 1.575 mmol) was dissolved in DMF (8 mL). To this solution, NaH (95 mg, 2.363 mmol) was added at 0° C., and the reaction was stirred at RT for 4 h. The reaction progress was monitored by TLC (10% MeOH in DCM). After completion, the reaction was quenched with ice-cold water (10 mL), and the mixture was extracted with EtOAc (2×70 mL). The combined organic layer was washed with water (2×10 mL), dried over anhydrous sodium sulfate and filtered, and the filtrate was concentrated under reduced pressure. The crude was purified by silica gel column chromatography using MeOH in DCM (product was eluted at 7-8% MeOH in DCM) to obtain benzyl isopropyl((1r,3r)-3-methyl-3-(2-oxopyrrolidin-1-yl)cyclobutyl)carbamate (390 mg, 69.7% yield) as a gummy liquid. ¹H NMR (400 MHz, DMSO-d₆): δ 7.50-7.29 (m, 5H), 5.08 (s, 2H), 4.14-3.89 (m, 2H), 3.39 (t, J=6.9 Hz, 2H), 2.72-2.61 (m, 2H), 2.36 (br t, J=10.4 Hz, 2H), 2.21 (t, J=8.1 Hz, 2H), 1.89 (quin, J=7.5 Hz, 2H), 1.19 (br s, 3H), 1.11 (d, J=6.9 Hz, 6H); LCMS (Method A): Rt=1.95 min, m/z=345.1 [M+H]⁺, 96.98%.

### Step 3. Benzyl isopropyl((1r,3r)-3-methyl-3-(pyrrolidin-1-yl)cyclobutyl)carbamate

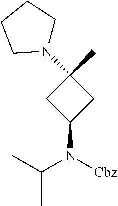 [SMILES: CC(C)N(C(=O)OCc1ccccc1)[C@H]1C[C@](C)(N2CCCC2)C1]

In a dried, 50 mL round bottom flask under a nitrogen atmosphere, benzyl isopropyl((1r,3r)-3-methyl-3-(2-oxopyrrolidin-1-yl)cyclobutyl)carbamate (340 mg, 0.987 mmol) was dissolved in THF (12 mL). To this solution, BH₃·THF (3.95 mL, 3.95 mmol, 1 M in THF) was added at 0° C., and the reaction was stirred at RT for 3 h, then at 50° C. for 3 h. The reaction progress was monitored by TLC (100% EtOAc). After completion, the reaction was concentrated under reduced pressure. The residue was dissolved in MeOH (12 mL) and the mixture was stirred at 60° C. for 8 h. The reaction was concentrated under reduced pressure to obtain crude benzyl isopropyl((1r,3r)-3-methyl-3-(pyrrolidin-1-yl)cyclobutyl)carbamate (300 mg, 92% yield) as a liquid. This material was used without further purification. LCMS (Method A): Rt=2.18 min, m/z=331.1 [M+H]⁺, 72.80%.

### Step 4. (1r,3r)—N-Isopropyl-3-methyl-3-(pyrrolidin-1-yl)cyclobutan-1-amine

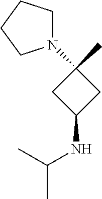

This compound was synthesized following the procedure described for the synthesis of Intermediate 38, step 2, starting with 1 equivalent of benzyl isopropyl((1r,3r)-3-methyl-3-(pyrrolidin-1-yl)cyclobutyl)carbamate, except that 0.31 equivalent of Pd—C was used. After filtration, the filtrate was concentrated under reduced pressure to obtain crude (1r,3r)—N-isopropyl-3-methyl-3-(pyrrolidin-1-yl)cyclobutan-1-amine as a gummy liquid. This material was used without further purification. This material was used without further purification.

Yield: 69%; ¹H NMR (400 MHz, DMSO-d₆): δ 3.97-3.83 (m, 1H), 3.26-3.10 (m, 2H), 2.76-2.60 (m, 2H), 2.39-2.27 (m, 2H), 1.71-1.57 (m, 5H), 1.49-1.40 (m, 2H), 1.12 (s, 3H), 0.93 (d, J=6.2 Hz, 6H), one proton merged with solvent peaks.

### Intermediate 41. tert-Butyl(2s,4r)-2-(isopropylamino)-5-azaspiro[3.4]octane-5-carboxylate (Procedure 1)

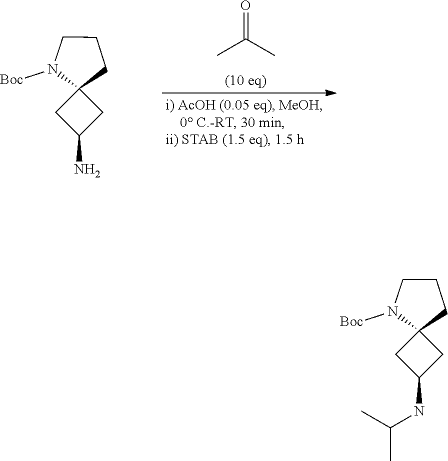

This compound was synthesized following the procedure described for the synthesis of Intermediate 1, starting with 1 equivalent of tert-butyl(2s,4r)-2-amino-5-azaspiro[3.4]octane-5-carboxylate, except that 10 equivalents of acetone, and 1.5 equivalents of STAB were used. After completion, the reaction was quenched with aqueous NaHCO₃ solution, and the mixture was extracted with EtOAc. The combined organic layer was dried over anhydrous sodium sulfate and filtered, and the filtrate was concentrated under reduced pressure to obtain crude tert-butyl(2s,4r)-2-(isopropylamino)-5-azaspiro[3.4]octane-5-carboxylate as a gummy liquid. This material was used without further purification.

Yield: 79%; ¹H NMR (400 MHz, DMSO-d₆): δ 3.55-3.45 (m, 1H), 3.23 (br t, J=6.6 Hz, 2H), 3.01-2.75 (m, 3H), 2.08-2.02 (m, 2H), 1.82-1.71 (m, 2H), 1.66-1.55 (m, 2H), 1.52-1.34 (m, 9H), 0.96 (br d, J=3.9 Hz, 6H), one proton merged with solvent peaks.

### Intermediate 41. tert-Butyl(2s,4r)-2-(isopropylamino)-5-azaspiro[3.4]octane-5-carboxylate (Procedure 2)

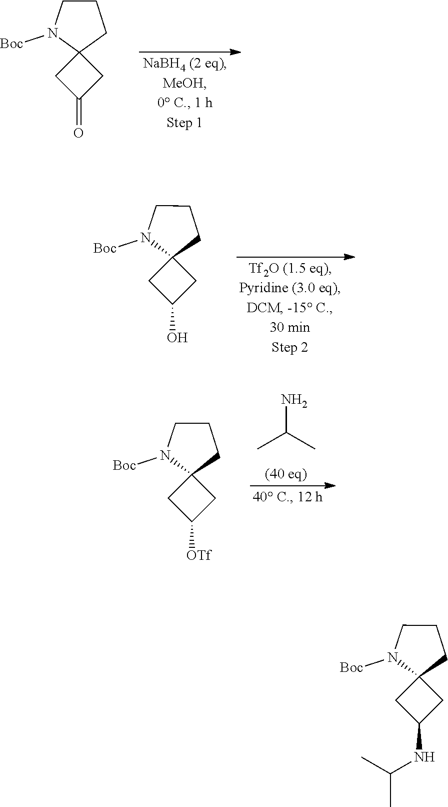

### Step 1. tert-Butyl(2r,4s)-2-hydroxy-5-azaspiro[3.4]octane-5-carboxylate

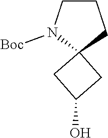 [SMILES: CC(C)(C)OC(=O)N1CCC[C@]12C[C@H](O)C2]

To a stirred solution of tert-butyl 2-oxo-5-azaspiro[3.4]octane-5-carboxylate (10 g, 44.4 mmol) in MeOH (100 mL), NaBH₄ (3.36 g, 89 mmol) was added over 20 min at 0° C., and the reaction was stirred at the same temperature for 1 h. The reaction progress was monitored by TLC (30/a EtOAc in hexane). After completion, the reaction was quenched with ice-cold water (150 mL), and the mixture was extracted with EtOAc (2×200 mL). The combined organic layer was washed with brine solution (100 mL), dried over anhydrous sodium sulfate and filtered, and the filtrate was concentrated under reduced pressure to obtain crude tert-butyl(2r,4s)-2-hydroxy-5-azaspiro[3.4]octane-5-carboxylate (14.2 g, quantitative yield) as a solid. This material was used without further purification. LCMS (Method A): Rt=1.68 min, m/z=172.1 [M+H−56]⁺, 98.69%.

### Step 2. tert-Butyl(2r,4s)-2-(((trifluoromethyl)sulfonyl)oxy)-5-azaspiro[3.4]octane-5-carboxylate

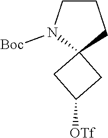 [SMILES: CC(C)(C)OC(=O)N1CCC[C@]12C[C@H](OS(=O)(=O)C(F)(F)F)C2]

To a stirred solution of tert-butyl(2r,4s)-2-hydroxy-5-azaspiro[3.4]octane-5-carboxylate (7 g, 30.8 mmol) in DCM (70 mL) and pyridine (7.46 mL, 92 mmol), Tf₂O (7.76 mL, 46.2 mmol) was added over 15 min at 0° C. The reaction was stirred at 0° C. for 30 min. The reaction progress was monitored by TLC (30% EtOAc in hexane). After completion, the reaction was quenched with ice-cold water (70 mL), and the mixture was extracted with DCM (2×100 mL). The combined organic layer was washed with aqueous NaHCO₃ solution (70 mL), dried over anhydrous sodium sulfate and filtered, and the filtrate was concentrated under reduced pressure to afford crude tert-butyl(2r,4s)-2-(((trifluoromethyl)sulfonyl)oxy)-5-azaspiro[3.4]octane-5-carboxylate (10 g, 14.39 mmol, 46.7% yield). This compound was used without further purification. LCMS (Method A): Rt=2.36 min, m/z=303.9 [M+H−56]⁺, 51.70%.

### Step 3. tert-Butyl(2s,4r)-2-(isopropylamino)-5-azaspiro[3.4]octane-5-carboxylate

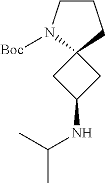 [SMILES: CC(C)N[C@H]1C[C@@]2(CCCN2C(=O)OC(C)(C)C)C1]

In 250 mL sealed tube, tert-butyl(2r,4s)-2-(((trifluoromethyl)sulfonyl)oxy)-5-azaspiro[3.4]octane-5-carboxylate (10 g, 27.8 mmol) was dissolved in isopropylamine (65.8 g, 1113 mmol) at RT, and the reaction was stirred at 40° C. for 12 h. The reaction progress was monitored by TLC (10% MeOH in DCM). After completion, the reaction was quenched with 10% NaHCO₃ solution (100 mL) and the mixture was extracted with 10% MeOH in DCM (3×100 mL). The combined organic layer was washed with brine solution (100 mL), dried over anhydrous sodium sulfate and filtered, and the filtrate was concentrated under reduced pressure to afford crude (tert-butyl(2s,4r)-2-(isopropylamino)-5-azaspiro[3.4]octane-5-carboxylate (10.5 g, quantitative yield) as a gummy liquid. This material was used without further purification. ¹H NMR (400 MHz, DMSO-d₆): δ 3.59-3.53 (m, 1H), 3.45-3.35 (m, 1H), 3.23 (t, J=6.6 Hz, 2H), 2.95-2.66 (m, 3H), 2.09-1.98 (m, 2H), 1.84-1.73 (m, 2H), 1.60 (quin, J=6.6 Hz, 2H), 1.53-1.34 (m, 9H), 1.05-0.86 (m, 6H); LCMS (Method B): Rt=1.25 min, m/z=269.6 [M+H]⁺, 83.12%.

Note: The structure was confirmed by ¹H—COSY and NOE studies.

### Intermediate 42. tert-Butyl (1-ethyl-3-(isopropylamino)cyclobutyl)carbamate

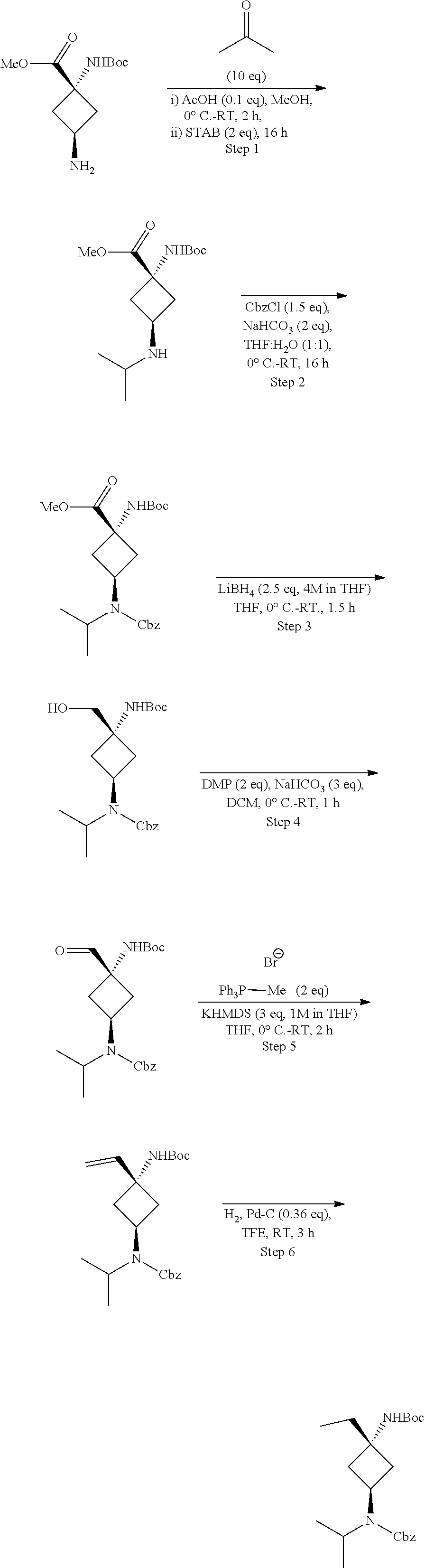

### Step 1. Methyl (1r,3r)-1-((tert-butoxycarbonyl)amino)-3-(isopropylamino)cyclobutane-1-carboxylate

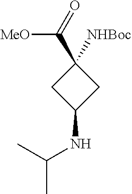 [SMILES: COC(=O)[C@]1(NC(=O)OC(C)(C)C)C[C@H](NC(C)C)C1]

This compound was synthesized following the procedure described for the synthesis of Intermediate 1, starting with 1 equivalent of methyl (1r,3r)-3-amino-1-((tert-butoxycarbonyl)amino)cyclobutane-1-carboxylate, except that 10 equivalents of acetone and 0.1 equivalent of AcOH were used. After work-up, the combined organic layer was concentrated under reduced pressure to obtain crude methyl (1r,3r)-1-((tert-butoxycarbonyl)amino)-3-(isopropylamino)cyclobutane-1-carboxylate as a gummy liquid. This material was used without further purification.

Yield: 99%; ¹H NMR (400 MHz, DMSO-d₆): δ 7.55 (br s, 1H), 3.59 (s, 3H), 3.30-3.23 (m, 1H), 2.71-2.63 (m, 1H), 2.40-2.30 (m, 2H), 2.16-2.05 (m, 2H), 1.44-1.26 (m, 9H), 0.92 (d, J=6.2 Hz, 6H), one proton merged with solvent peaks.

### Step 2. Methyl (1r,3r)-3-(((benzyloxy)carbonyl)(isopropyl)amino)-1-((tert-butoxycarbonyl)amino)cyclobutane-1-carboxylate

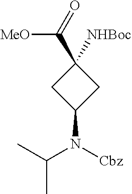 [SMILES: COC(=O)[C@]1(NC(=O)OC(C)(C)C)C[C@H](N(C(=O)OCc2ccccc2)C(C)C)C1]

This compound was synthesized following the procedure described for the synthesis of Intermediate 37, Step 1, starting with 1 equivalent of methyl (1r,3r)-1-((tert-butoxycarbonyl)amino)-3-(isopropylamino)cyclobutane-1-carboxylate, except that 2 equivalents of NaHCO₃ was used. After completion, the reaction was quenched with NaHCO₃ solution, and the mixture was extracted with EtOAc. The combined organic layer was washed with brine solution, dried over anhydrous sodium sulfate and filtered, and the filtrate was concentrated under reduced pressure. The crude was purified by silica gel flash column chromatography using EtOAc in hexane (product was eluted at 15% EtOAc in hexane) to obtain methyl (1r,3r)-3-(((benzyloxy)carbonyl)(isopropyl)amino)-1-((tert-butoxycarbonyl)amino)cyclobutane-1-carboxylate as a gummy liquid.

Yield: 64.2%; LCMS (Method E): Rt=2.62 min, m/z=321.2 [M+H−100]⁺, 50.25%.

### Step 3. Benzyl ((1r,3r)-3-((tert-butoxycarbonyl)amino)-3-(hydroxymethyl)cyclobutyl) (isopropyl)carbamate

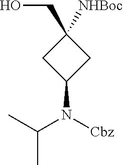 [SMILES: CC(C)N(C(=O)OCc1ccccc1)[C@H]1C[C@](CO)(NC(=O)OC(C)(C)C)C1]

In a 100 mL two-neck dried round bottom flask under a nitrogen atmosphere, methyl (1r,3r)-3-(((benzyloxy)carbonyl)(isopropyl)amino)-1-((tert-butoxycarbonyl)amino)cyclobutane-1-carboxylate (1.4 g, 3.33 mmol) was dissolved in THF (30 mL). To this solution, lithium borohydride (2.081 mL, 8.32 mmol, 4 M in THF) was added at 0° C., and the reaction was stirred at RT for 1.5 h. The reaction progress was monitored by TLC (30% EtOAc in hexane). After completion, the reaction was quenched with EtOAc (1 mL) at 0° C., the mixture was filtered through a Celite® pad, and the pad was washed with EtOAc (2×40 mL). The filtrate was concentrated under reduced pressure to obtain crude benzyl ((1r,3r)-3-((tert-butoxycarbonyl)amino)-3-(hydroxymethyl)cyclobutyl)(isopropyl)carbamate (1.3 g, 66.1% yield) as a gummy liquid. This material was used without further purification. LCMS (Method E): Rt=2.04 min, m/z=293.0 [M+H−100]⁺, 47.61%.

### Step 4. Benzyl ((1r,3r)-3-((tert-butoxycarbonyl)amino)-3-formylcyclobutyl)(isopropyl)carbamate

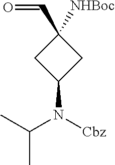 [SMILES: CC(C)N(C(=O)OCc1ccccc1)[C@H]1C[C@](C=O)(NC(=O)OC(C)(C)C)C1]

In a 100 mL two-neck dried round bottom flask under a nitrogen atmosphere, benzyl ((1r,3r)-3-((tert-butoxycarbonyl)amino)-3-(hydroxymethyl)cyclobutyl)(isopropyl)carbamate (1 g, 2.55 mmol) was dissolved in DCM (30 mL). To this solution, sodium bicarbonate (0.642 g, 7.64 mmol) and DMP (2.161 g, 5.10 mmol) were added at 0° C., and the reaction was stirred at RT for 1 h. The reaction progress was monitored by TLC (30% EtOAc in hexane). After completion, the reaction was quenched with NaHCO₃ solution (30 mL), and the reaction was extracted with DCM (3×50 mL). The combined organic layer was washed with brine solution (40 mL), dried over anhydrous sodium sulfate and filtered, and the filtrate was concentrated under reduced pressure. The crude was purified by silica gel flash column chromatography using EtOAc in hexane (product was eluted at 35% EtOAc in hexane) to obtain benzyl ((1r,3r)-3-((tert-butoxycarbonyl)amino)-3-formylcyclobutyl)(isopropyl)carbamate (900 mg, 74.1% yield) as a solid. ¹H NMR (400 MHz, DMSO-d₆): δ 9.31 (br s, 1H), 7.90 (s, 1H), 7.43-7.29 (m, 5H), 5.09 (s, 2H), 4.45-4.25 (m, 1H), 3.97-3.87 (m, 1H), 2.79-2.66 (m, 2H), 2.22-2.11 (m, 2H), 1.45-1.27 (m, 9H), 1.16 (d, J=5.8 Hz, 6H); LCMS (Method A): Rt=2.21 min, m/z=291.0 [M+H−100]⁺, 81.95%.

### Step 5. Benzyl ((1r,3r)-3-((tert-butoxycarbonyl)amino)-3-vinylcyclopentyl)(isopropyl)carbamate

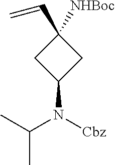 [SMILES: C=C[C@]1(NC(=O)OC(C)(C)C)C[C@H](N(C(=O)OCc2ccccc2)C(C)C)C1]

In a 100 mL two-neck dried round bottom flask under a nitrogen atmosphere, methyltriphenylphosphonium bromide (1555 mg, 4.35 mmol) was dissolved in THF (10 mL). To this solution, potassium bis(trimethylsilyl)amide (6.53 mL, 6.53 mmol, 1 M in THF) was added, and the reaction was stirred at 0° C. for 30 min. To this reaction, benzyl ((1r,3r)-3-((tert-butoxycarbonyl)amino)-3-formylcyclobutyl)(isopropyl)carbamate (850 mg, 2.177 mmol, in 10 mL THF) was added at 0° C., and the reaction was stirred at RT for 2 h. The reaction progress was monitored by TLC (20% EtOAc in hexane). After completion, the reaction was quenched with aqueous NH₄Cl solution (30 mL), and the mixture was extracted with EtOAc (3×50 mL). The combined organic layer was washed with brine solution (50 mL), dried over anhydrous sodium sulfate and filtered, and the filtrate was concentrated under reduced pressure to obtain crude benzyl ((1r,3r)-3-((tert-butoxycarbonyl)amino)-3-vinylcyclobutyl)(isopropyl)carbamate (800 mg, 82% yield) as a semi-solid. This material was used without further purification. LCMS (Method H): Rt=2.30 min, m/z=389.2 [M+H]⁺, 98.53%.

### Step 6. tert-Butyl ((1r,3s)-1-ethyl-3-(isopropylamino)cyclobutyl)carbamate

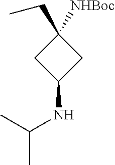 [SMILES: CC[C@]1(NC(=O)OC(C)(C)C)C[C@H](NC(C)C)C1]

This compound was synthesized following the procedure described for the synthesis of Intermediate 38, Step 2, starting with 1 equivalent of benzyl (3-((tert-butoxycarbonyl)amino)-3-vinylcyclobutyl)(isopropyl)carbamate, except that 0.36 equivalent of Pd—C was used. After filtration, the filtrate was concentrated under reduced pressure to obtain crude tert-butyl ((1r,3s)-1-ethyl-3-(isopropylamino)cyclobutyl)carbamate as a gummy liquid. This material was used without further purification.

Yield: 82% yield; ¹H NMR (400 MHz, DMSO-d₆): δ 6.78 (br s, 1H), 3.25-3.19 (m, 1H), 2.75-2.63 (m, 1H), 2.41-2.32 (m, 2H), 1.64 (q, J=7.4 Hz, 2H), 1.53-1.44 (m, 2H), 1.38 (s, 9H), 0.92 (d, J=6.3 Hz, 6H), 0.67 (t, J=7.4 Hz, 3H), one proton merged with solvent peaks.

### Intermediate 43. 5-Fluoro-2-hydroxy-N-isopropyl-N-((1r,3r)-3-methyl-3-(pyrrolidin-1-yl)cyclobutyl)benzamide

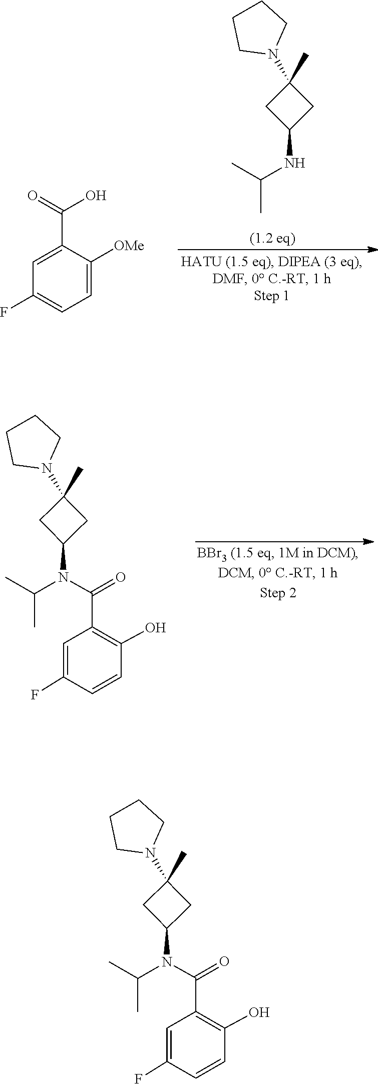 [SMILES: COc1ccc(F)cc1C(=O)O ; CC(C)N[C@H]1C[C@](C)(N2CCCC2)C1 ; CC(C)N(C(=O)c1cc(F)ccc1O)[C@H]1C[C@](C)(N2CCCC2)C1 ; CC(C)N(C(=O)c1cc(F)ccc1O)[C@H]1C[C@](C)(N2CCCC2)C1]

### Step 1. 5-Fluoro-N-isopropyl-2-methoxy-N-((1r,3r)-3-methyl-3-(pyrrolidin-1-yl)cyclobutyl)benzamide

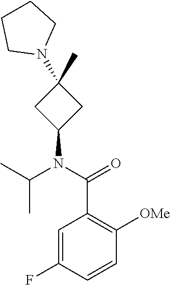 [SMILES: COc1ccc(F)cc1C(=O)N(C(C)C)[C@H]1C[C@](C)(N2CCCC2)C1]

This compound was synthesized following the procedure described for the synthesis of Intermediate 35, Step 1, starting with 1 equivalent of 5-fluoro-2-methoxybenzoic acid and 1.2 equivalents of (1r,3r)—N-isopropyl-3-methyl-3-(pyrrolidin-1-yl)cyclobutan-1-amine. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain 5-fluoro-N-isopropyl-2-methoxy-N-((1r,3r)-3-methyl-3-(pyrrolidin-1-yl)cyclobutyl)benzamide as a gummy liquid.

Yield: 47.7%; LCMS (Method B): Rt=1.13 min, m/z=349.2 [M+H]⁺, 90.40%.

### Step 2. 5-Fluoro-2-hydroxy-N-isopropyl-N-((1r,3r)-3-methyl-3-(pyrrolidin-1-yl)cyclobutyl)benzamide

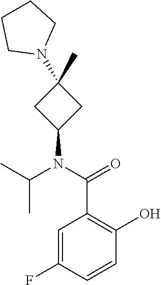 [SMILES: CC(C)N(C(=O)c1cc(F)ccc1O)[C@H]1C[C@](C)(N2CCCC2)C1]

This compound was synthesized following the procedure described for the synthesis of Intermediate 35, step 2, starting with 1 equivalent of 5-fluoro-N-isopropyl-2-methoxy-N-((1r,3r)-3-methyl-3-(pyrrolidin-1-yl)cyclobutyl)benzamide, except that 1.5 equivalents of BBr₃ (IM in DCM) was used. After completion, the reaction was quenched with 10% aqueous NaHCO₃ solution and the mixture was extracted with DCM. The combined organic layer was dried over anhydrous sodium sulfate and filtered, and the filtrate was concentrated under reduced pressure to obtain crude 5-fluoro-2-hydroxy-N-isopropyl-N-((1r,3r)-3-methyl-3-(pyrrolidin-1-yl)cyclobutyl)benzamide as a solid. This material was used without further purification.

Yield: 78%; LCMS (Method A): Rt=1.46 min, m/z=335.1 [M+H]⁺, 87.75%.

### Intermediate 44. 5-Fluoro-2-hydroxy-N-isopropyl-N-((2s,4r)-5-methyl-5-azaspiro[3.4]octan-2-yl)benzamide

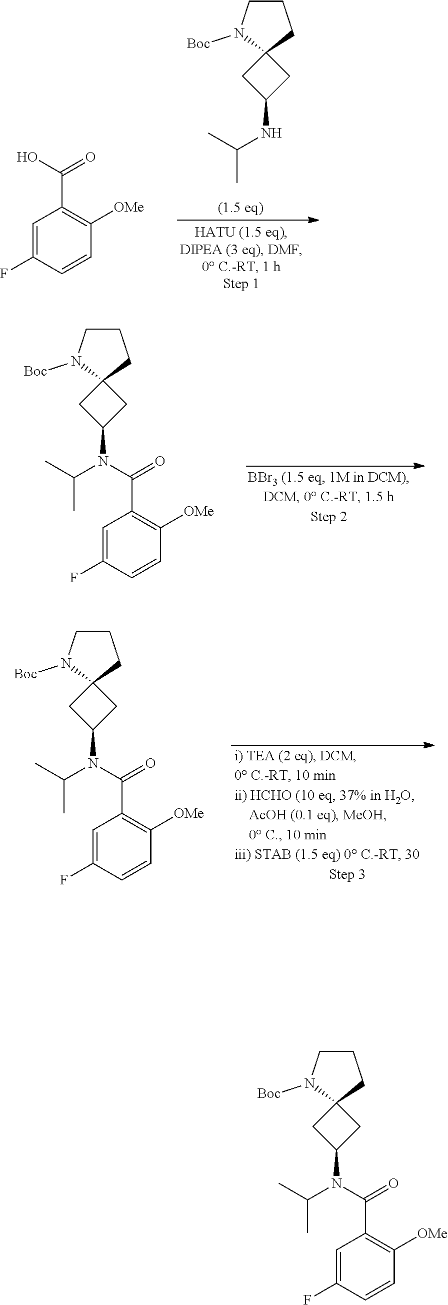 [SMILES: COc1ccc(F)cc1C(=O)O ; CC(C)N[C@H]1C[C@@]2(CCCN2C(=O)OC(C)(C)C)C1 ; COc1ccc(F)cc1C(=O)N(C(C)C)[C@H]1C[C@@]2(CCCN2C(=O)OC(C)(C)C)C1 ; COc1ccc(F)cc1C(=O)N(C(C)C)[C@H]1C[C@@]2(CCCN2C(=O)OC(C)(C)C)C1 ; COc1ccc(F)cc1C(=O)N(C(C)C)[C@H]1C[C@@]2(CCCN2C(=O)OC(C)(C)C)C1]

### Step 1. tert-Butyl(2s,4r)-2-(5-fluoro-N-isopropyl-2-methoxybenzamido)-5-azaspiro[3.4]octane-5-carboxylate

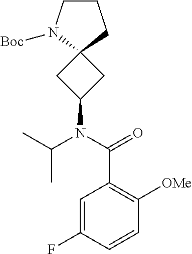 [SMILES: COc1ccc(F)cc1C(=O)N(C(C)C)[C@H]1C[C@@]2(CCCN2C(=O)OC(C)(C)C)C1]

This compound was synthesized following the procedure described for the synthesis of Intermediate 35, step 1, starting with 1 equivalent of 5-fluoro-2-methoxybenzoic acid and 1.5 equivalents of tert-butyl(2s,4r)-2-(isopropylamino)-5-azaspiro[3.4]octane-5-carboxylate. After work-up, the crude was purified by silica gel flash column chromatography using EtOAc in hexane (product eluted at 15% EtOAc in hexane) to obtain tert-butyl(2s,4r)-2-(5-fluoro-N-isopropyl-2-methoxybenzamido)-5-azaspiro[3.4]octane-5-carboxylate as a gummy liquid.

Yield: 72.6%; ¹H NMR (400 MHz, DMSO-d₆): δ 7.25-7.12 (m, 1H), 7.11-6.91 (m, 2H), 4.47-4.02 (m, 1H), 3.81-3.67 (m, 3H), 3.61-3.45 (m, 1H), 3.30-3.18 (m, 3H), 3.03-2.88 (m, 1H), 2.74-2.63 (m, 2H), 2.29-1.91 (m, 2H), 1.72-1.36 (m, 11H), 1.16-0.90 (m, 6H); LCMS (Method A): Rt=2.30 min, m/z=421.2 [M+H]⁺, 95.42%.

### Step 2. 5-Fluoro-2-hydroxy-N-isopropyl-N-((2s,4r)-5-azaspiro[3.4]octan-2-yl)benzamide Hydrobromide

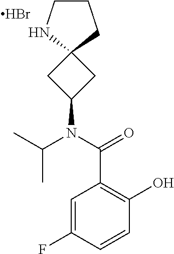 [SMILES: Br ; CC(C)N(C(=O)c1cc(F)ccc1O)[C@H]1C[C@@]2(CCCN2)C1]

This compound was synthesized following the procedure described for the synthesis of Intermediate 35, step 2, starting with 1 equivalent of tert-butyl(2s,4r)-2-(5-fluoro-N-isopropyl-2-methoxybenzamido)-5-azaspiro[3.4]octane-5-carboxylate, except that 1.5 equivalents of BBr₃ was used. After completion, the reaction was quenched with MeOH at 0° C. The crude was re-concentrated from MeOH and the residue was dried under reduced pressure to obtain crude 5-fluoro-2-hydroxy-N-isopropyl-N-((2s,4r)-5-azaspiro[3.4]octan-2-yl)benzamide hydrobromide as a gummy liquid. This material was used without further purification.

Yield: 93% yield; LCMS (Method A): Rt=1.27 min, m/z=307.1 [M+H]⁺, 66.51%.

### Step 3. 5-Fluoro-2-hydroxy-N-isopropyl-N-((2s,4r)-5-methy-5-azaspiro[3.4]octan-2-yl)benzamide

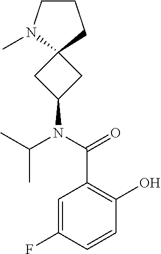 [SMILES: CC(C)N(C(=O)c1cc(F)ccc1O)[C@H]1C[C@@]2(CCCN2C)C1]

This compound was synthesized following the procedure described for the synthesis of Intermediate 1, starting with 1 equivalent of 5-fluoro-2-hydroxy-N-isopropyl-N-((2s,4r)-5-azaspiro[3.4]octan-2-yl)benzamide hydrobromide, except that 2 equivalents of TEA, 10 equivalents of formaldehyde (37% in H₂O), 0.1 equivalent of AcOH, and 1.5 equivalents of STAB were used. After work-up, the crude was purified by reverse phase chromatography using a SiliCycle SiliaSep C18 column (120 g) on Biotage isolera one (Mobile phase A: 10 mM NH₄HCO₃ in water, B: ACN) to afford 5-fluoro-2-hydroxy-N-isopropyl-N-((2s,4r)-5-methyl-5-azaspiro[3.4]octan-2-yl)benzamide as a solid.

Yield: 48.3%; ¹H NMR (400 MHz, DMSO-d₆): δ 9.62 (s, 1H), 7.08-6.98 (m, 1H), 6.92-6.79 (m, 2H), 3.95-3.76 (m, 1H), 3.72-3.59 (m, 1H), 2.49-2.42 (m, 2H), 2.33-2.11 (m, 5H), 1.94-1.76 (m, 2H), 1.55 (quin, J=7.4 Hz, 2H), 1.39-0.99 (m, 6H), two protons merged with solvent peaks; LCMS (Method A): Rt=1.50 min, m/z=321.4 [M+H]⁺, 97.11%.

### Intermediate 45. (±)-2-((4-(6-((6-Benzyl-5-methyl-5,6,7,8-tetrahydro-1,6-naphthyridin-4-yl)oxy)-2-azaspiro[3.3]heptan-2-yl)pyrimidin-5-yl)oxy)-5-fluorobenzoic Acid

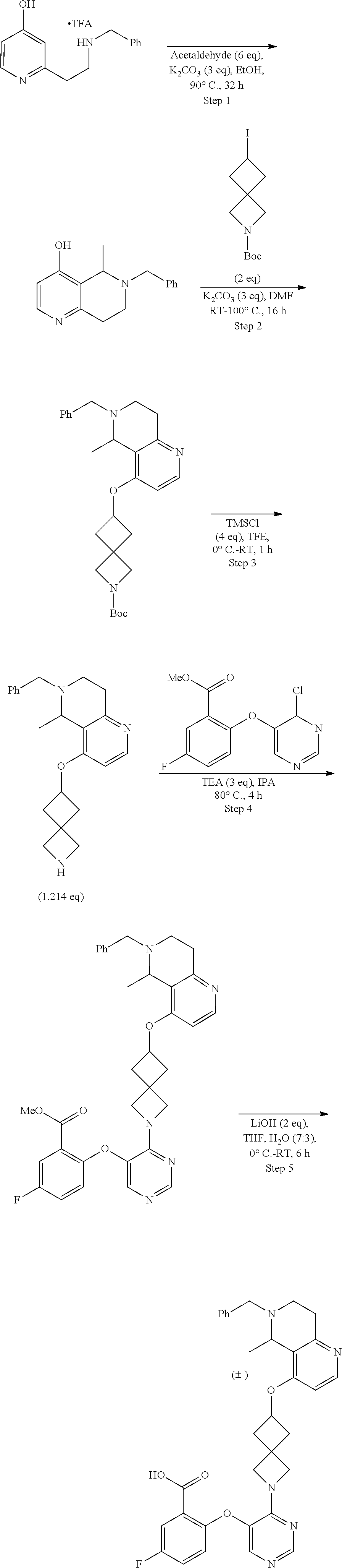

### Step 1. (±)-6-Benzyl-5-methyl-5,6,7,8-tetrahydro-1,6-naphthyridin-4-ol

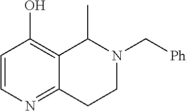 [SMILES: CC1c2c(O)ccnc2CCN1Cc1ccccc1]

The reaction was performed following the same procedure in four batches (4×10 g).

To a stirred solution of 2-(2-(benzylamino)ethyl)pyridin-4-ol 2,2,2-trifluoroacetic acid (10 g, 29.2 mmol) in EtOH (100 mL), potassium carbonate (12.11 g, 88 mmol) and acetaldehyde (4.95 mL, 88 mmol) were added at RT. The reaction was stirred at 90° C. for 16 h under a nitrogen atmosphere. The starting material was not consumed by TLC (10% MeOH in DCM), therefore another portion of acetaldehyde (4.95 mL, 88 mmol) was added at RT, and the reaction was stirred at 90° C. for 16 h. The reaction progress was monitored by TLC (10% MeOH in DCM). The reaction mixtures from four batches were combined and filtered through a Celite® pad, and the pad was washed with MeOH (2×200 mL). The filtrate was concentrated under reduced pressure. The crude product was purified by silica gel flash column chromatography using MeOH in DCM (product eluted at 20% MeOH in DCM) to afford (±)-6-benzyl-5-methyl-5,6,7,8-tetrahydro-1,6-naphthyridin-4-ol (18.5 g, 62.23% combined yield) as a solid. LCMS (Method A): Rt=1.30 min, m/z=255.2 [M+H]⁺, 90.57%.

Note: Traces of TFA remained with the product.

### Step 2. (±)-tert-Butyl 6-((6-benzyl-5-methyl-5,6,7,8-tetrahydro-1,6-naphthyridin-4-yl)oxy)-2-azaspiro[3.3]heptane-2-carboxylate

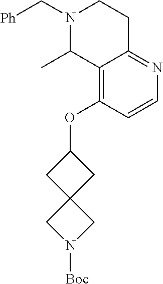 [SMILES: CC1c2c(OC3CC4(C3)CN(C(=O)OC(C)(C)C)C4)ccnc2CCN1Cc1ccccc1]

The reaction was performed following the same procedure in two batches.

This compound was synthesized following the procedure described for the synthesis of Intermediate 16, step 2, starting with 1 equivalent of (±)-6-benzyl-5-methyl-5,6,7,8-tetrahydro-1,6-naphthyridin-4-ol and 2 equivalents of tert-butyl 6-iodo-2-azaspiro[3.3]heptane-2-carboxylate. The reaction mixtures from both batches were combined and diluted with ice cold water, and the mixture was extracted with EtOAc. The aqueous layer was re-extracted with 10% MeOH in DCM. The combined organic layer was washed with cold water, dried over anhydrous sodium sulfate, and filtered, and the filtrate was concentrated under reduced pressure. The crude product was purified by silica gel flash column chromatography using MeOH in DCM (product eluted at 8% MeOH in DCM) to obtain (±)-tert-butyl 6-((6-benzyl-5-methyl-5,6,7,8-tetrahydro-1,6-naphthyridin-4-yl)oxy)-2-azaspiro[3.3]heptane-2-carboxylate as a sticky solid.

Combined yield: 55.64%; LCMS (Method B): Rt=1.48 min, m/z=450.2 [M+H]⁺, 92.41%.

### Step 3. (k)-4-((2-Azaspiro[3.3]heptan-6-yl)oxy)-6-benzyl-S-methyl-5,6,7,8-tetrahydro-1,6-naphthyridine

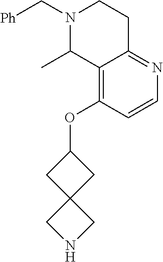 [SMILES: CC1c2c(OC3CC4(CNC4)C3)ccnc2CCN1Cc1ccccc1]

To a stirred solution of (±)-tert-butyl 6-((6-benzyl-5-methyl-5,6,7,8-tetrahydro-1,6-naphthyridin-4-yl)oxy)-2-azaspiro[3.3]heptane-2-carboxylate (12.5 g, 27.8 mmol) in TFE (130 mL), TMSCl (14.21 mL 111 mmol) was added at 0° C. The reaction was stirred at RT for 1 h. After completion, the reaction was concentrated under reduced pressure. The residue was dissolved in saturated aqueous sodium bicarbonate solution (50 mL) and the mixture was extracted with 10/a MeOH in DCM (3×50 mL). The combined organic layer was dried over anhydrous sodium sulfate and filtered, and the filtrate was concentrated under reduced pressure to afford crude (±)-4-((2-azaspiro[3.3]heptan-6-yl)oxy)-6-benzyl-5-methyl-5,6,7,8-tetrahydro-1,6-naphthyridine (10.7 g, 78% yield) as a sticky liquid. This material was used without further purification. LCMS (Method A): Rt=1.41 min, m/z=350.1 [M+H-Boc]⁺, 71.15%.

### Step 4. (±)-Methyl 2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluorobenzoate

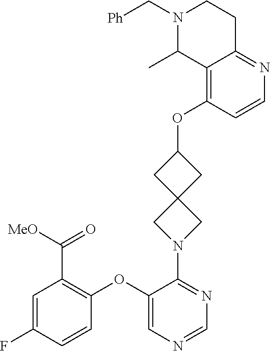 [SMILES: COC(=O)c1cc(F)ccc1Oc1cncnc1N1CC2(CC(Oc3ccnc4c3C(C)N(Cc3ccccc3)CC4)C2)C1]

This compound was synthesized following the procedure described for the synthesis of Intermediate 22, step 1, starting with 1 equivalent of methyl 2-((4-chloropyrimidin-5-yl)oxy)-5-fluorobenzoate and 1.214 equivalents of (±)-4-((2-azaspiro[3.3]heptan-6-yl)oxy)-6-benzyl-5-methyl-5,6,7,8-tetrahydro-1,6-naphthyridine. After completion, the reaction was concentrated under reduced pressure. The crude compound was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure and lyophilized to afford (±)-methyl 2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluorobenzoate as a sticky solid.

Yield: 80%; ¹H NMR (400 MHz, DMSO-d₆): δ 8.29 (s, 1H), 8.19 (d, J=5.6 Hz, 1H), 7.69-7.63 (m, 2H), 7.48 (ddd, J=3.3, 7.8, 9.1 Hz, 1H), 7.35-7.29 (m, 4H), 7.27-7.21 (m, 1H), 7.11 (dd, J=4.4, 9.1 Hz, 1H), 6.68 (d, J=5.8 Hz, 1H), 4.73 (quin, J=6.6 Hz, 1H), 4.26 (s, 2H), 4.17 (s, 2H), 3.96 (q, J=6.4 Hz, 1H), 3.81 (s, 3H), 3.77-3.72 (m, 1H), 3.60 (d, J=13.8 Hz, 1H), 3.10-2.99 (m, 1H), 2.92-2.68 (m, 4H), 2.65-2.54 (m, 1H), 2.29-2.15 (m 2H), 1.23 (d, J=6.5 Hz, 3H); LCMS (Method A): Rt=2.14 min, m/z=596.2 [M+H]⁺, 98.37%.

### Step 5. (±)-2-((4-(6-((6-Benzyl-5-methyl-5,6,7,8-tetrahydro-1,6-naphthyridin-4-yl)oxy)-2-azaspiro[3.3]heptan-2-yl)pyrimidin-5-yl)oxy)-5-fluorobenzoic Acid

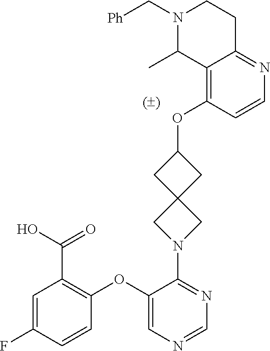 [SMILES: CC1c2c(OC3CC4(C3)CN(c3ncncc3Oc3ccc(F)cc3C(=O)O)C4)ccnc2CCN1Cc1ccccc1]

In a 500 mL two neck round bottom flask, (±)-methyl 2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluorobenzoate (6 g, 10.07 mmol) was dissolved in THF (120 mL) and water (51.4 mL). To this solution, LiOH·H₂O (0.845 g, 20.15 mmol) was added at 0° C. The reaction was stirred at RT for 6 h. The reaction progress was monitored by LCMS and TLC (10% MeOH in DCM). After completion, the reaction was concentrated under reduced pressure. The residue was diluted with water (5 mL), acidified with 1.5N HCl (˜pH-2), then neutralized using solid NaHCO₃ (˜pH-7). The mixture was extracted with 10% MeOH in DCM (2×25 mL). The combined organic layer was dried over anhydrous sodium sulfate and filtered, and the filtrate was concentrated under reduced pressure to obtain crude (±)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluorobenzoic acid (5.1 g, 70.7% yield) as a solid. This material was used without further purification. LCMS (Method A): Rt=1.44 min, m/z=582.1 [M+H]⁺, 81.23%.

### Intermediate 46. (±)-2-((4-(6-((6-Benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluorobenzoic Acid

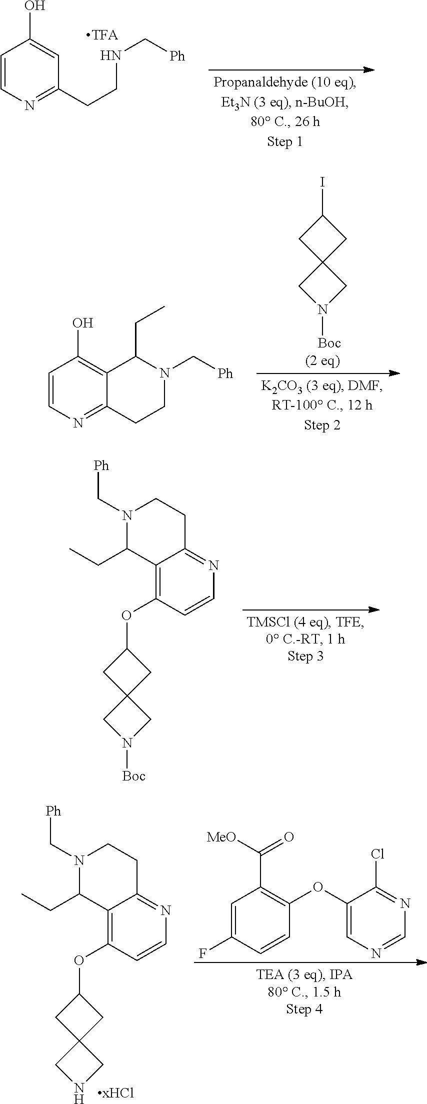

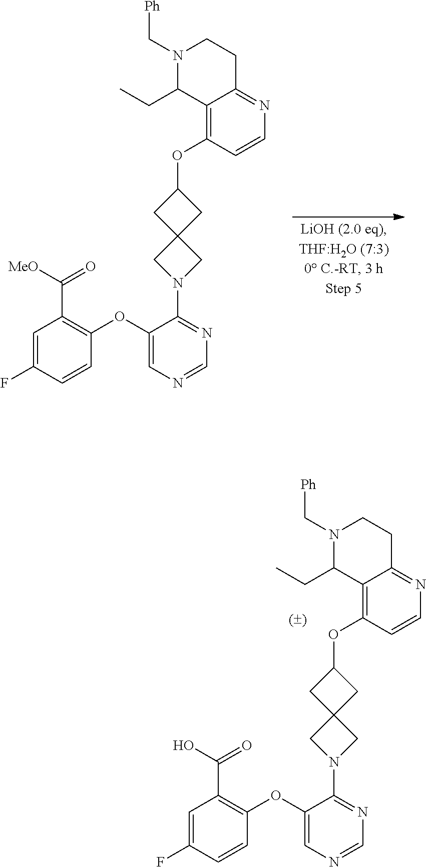 [SMILES: CCC1c2c(OC3CC4(C3)CN(c3ncncc3Oc3ccc(F)cc3C(=O)OC)C4)ccnc2CCN1Cc1ccccc1 ; CCC1c2c(OC3CC4(C3)CN(c3ncncc3Oc3ccc(F)cc3C(=O)O)C4)ccnc2CCN1Cc1ccccc1]

### Step 1. (±)-6-Benzyl-5-ethyl-5,6,7,8-tetrahydro-1,6-naphthyridin-4-ol

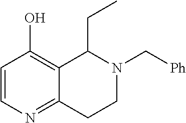 [SMILES: CCC1c2c(O)ccnc2CCN1Cc1ccccc1]

In a dried, 500 mL two neck round bottom flask under a nitrogen atmosphere, 2-(2-(benzylamino)ethyl)pyridin-4-ol 2,2,2-trifluoroacetate (24 g, 70.1 mmol) was dissolved in n-butanol (200 mL). To this solution, TEA (29.6 mL, 210 mmol) and propionaldehyde (25.1 mL, 351 mmol) were added at RT, and the reaction was stirred at 80° C. for 12 h. The starting material was not consumed. Therefore, another portion of propionaldehyde (25.1 mL, 351 mmol) was added at RT and the reaction was stirred at 80° C. for 12 h. The reaction progress was monitored by LCMS. After completion, the reaction was concentrated under reduced pressure. The residue was diluted with 10% aqueous NaHCO₃ solution (500 mL) and extracted with 10% MeOH in DCM (3×500 mL). The combined organic layer was washed with water (500 mL), dried over anhydrous sodium sulfate and filtered, and the filtrate was concentrated under reduced pressure. The crude compound was triturated with EtOAc (200 mL). The organic layer was decanted and the solid was dried under vacuum to obtain (±)-6-benzyl-5-ethyl-5,6,7,8-tetrahydro-1,6-naphthyridin-4-ol (7.5 g, 38.4% yield) as a solid. ¹H NMR (400 MHz, DMSO-d₆): δ 11.21 (br s, 1H), 7.49 (br d, J=6.6 Hz, 1H), 7.39-7.16 (m, 5H), 5.94 (br d, J=6.6 Hz, 1H), 3.77-3.48 (m, 2H), 3.39-3.36 (m, 1H), 3.22-3.05 (m, 1H), 2.81-2.70 (m, 2H), 2.31-2.19 (m, 1H), 1.76-1.57 (m, 1H), 1.47-1.30 (m, 1H), 0.87 (t, J=7.3 Hz, 3H); LCMS (Method A): Rt=2.94 min, m/z=269.0 [M+H]⁺, 96.33%.

### Step 2. (t)-tert-Butyl 6-((6-benzyl-5-ethyl-5,6,7,8-tetrahydro-1,6-naphthyridin-4-yl)oxy)-2-azaspiro[3.3]heptane-2-carboxylate

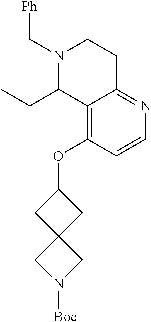 [SMILES: CCC1c2c(OC3CC4(C3)CN(C(=O)OC(C)(C)C)C4)ccnc2CCN1Cc1ccccc1]

The reaction was performed following the same procedure in two batches.

This compound was synthesized following the procedure described for the synthesis of Intermediate 16, step 2, starting with 1 equivalent of (±)-6-benzyl-5-ethyl-5,6,7,8-tetrahydro-1,6-naphthyridin-4-ol and 2 equivalents of tert-butyl 6-iodo-2-azaspiro[3.3]heptane-2-carboxylate. After completion, the reaction mixture was diluted with EtOAc and filtered through a Celite® pad, and the pad was washed with EtOAc. The filtrate was diluted with ice cold water, and the mixture was extracted with EtOAc. The combined organic layer was dried over anhydrous sodium sulfate and filtered, and the filtrate was concentrated under reduced pressure. The crude from both batches were combined and purified by silica gel flash column chromatography using EtOAc in hexane (product eluted at 100% EtOAc) to obtain (f)-tert-butyl 6-((6-benzyl-5-ethyl-5,6,7,8-tetrahydro-1,6-naphthyridin-4-yl)oxy)-2-azaspiro[3.3]heptane-2-carboxylate as a gummy liquid.

Combined yield: 64.5%; ¹H NMR (400 MHz, DMSO-d₆): δ 8.21 (d, J=5.5 Hz, 1H), 7.37-7.21 (m, 5H), 6.67 (d, J=5.7 Hz, 1H), 4.70 (quin, J=6.4 Hz, 1H), 3.89 (br s, 2H), 3.81 (br s, 2H), 3.75-3.65 (m, 1H), 3.63-3.54 (m, 2H), 3.21-3.11 (m, 1H), 2.98-2.86 (m, 1H), 2.84-2.69 (m, 3H), 2.21-2.07 (m, 2H), 1.67-1.50 (m, 2H), 1.37 (s, 9H), 0.92 (t, J=7.2 Hz, 3H), one proton merged with solvent peaks; LCMS (Method A): Rt=2.52 min, m/z=464.2 [M+H]⁺, 99.40%.

### Step 3. (±)-4-((2-Azaspiro[3.3]heptan-6-yl)oxy)-6-benzyl-5-ethyl-5,6,7,8-tetrahydro-1,6-naphthyridine xHCl

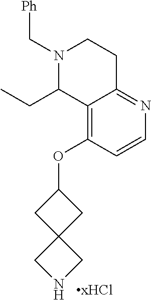 [SMILES: CCC1c2c(OC3CC4(CNC4)C3)ccnc2CCN1Cc1ccccc1]

The reaction was performed following the same procedure in two batches.

This compound was synthesized following the procedure described for the synthesis of Intermediate 16, step 3, starting with 1 equivalent of (±)-tert-butyl 6-((6-benzyl-5-ethyl-5,6,7,8-tetrahydro-1,6-naphthyridin-4-yl)oxy)-2-azaspiro[3.3]heptane-2-carboxylate. After completion, the crude from both batches were combined and concentrated under reduced pressure. The residue was triturated with EtOAc. The organic layer was decanted and the solid was dried under vacuum to obtain crude (±)-4-((2-azaspiro[3.3]heptan-6-yl)oxy)-6-benzyl-5-ethyl-5,6,7,8-tetrahydro-1,6-naphthyridine xHCl as a solid. This material was used without further purification.

Quantitative yield; LCMS (Method H): Rt=1.04 min, m/z=364.4 [M+H]⁺, 96.46%.

### Step 4. (±)-Methyl 2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluorobenzoate

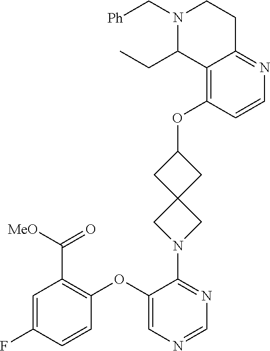 [SMILES: CCC1c2c(OC3CC4(C3)CN(c3ncncc3Oc3ccc(F)cc3C(=O)OC)C4)ccnc2CCN1Cc1ccccc1]

The reaction was performed following the same procedure in two batches.

This compound was synthesized following the procedure described for the synthesis of Intermediate 22, step 1, starting with 1 equivalent of (±)-methyl 2-((4-chloropyrimidin-5-yl)oxy)-5-fluorobenzoate and 1 equivalent of 4-((2-azaspiro[3.3]heptan-6-yl)oxy)-6-benzyl-5-ethyl-5,6,7,8-tetrahydro-1,6-naphthyridine xHCl. After completion, the reaction was concentrated under reduced pressure, the residue was diluted with water, and the mixture was extracted with EtOAc. The combined organic layer was dried over anhydrous sodium sulfate and filtered, and the filtrate was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN) to obtain (t)-methyl 2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluorobenzoate as a solid.

Yield: 70%; ¹H NMR (400 MHz, DMSO-d₆): δ 8.29 (s, 1H), 8.20 (d, J=5.6 Hz, 1H), 7.70-7.62 (m, 2H), 7.53-7.44 (m, 1H), 7.37-7.18 (m, 5H), 7.12 (dd, J=4.5, 9.1 Hz, 1H), 6.68 (d, J=5.6 Hz, 1H), 4.74 (quin, J=6.4 Hz, 1H), 4.26 (s, 2H), 4.18 (s, 2H), 3.80 (s, 3H), 3.73-3.49 (m, 31H), 3.23-3.11 (m, 11H), 2.97-2.85 (m, 1H), 2.83-2.71 (m, 3H), 2.49-2.44 (m, 1H), 2.26-2.10 (m, 2H), 1.67-1.47 (m, 2H), 0.90 (t, J=7.3 Hz, 3H); LCMS (Method A): Rt=2.30 min, m/z=610.2 [M+H]⁺; HPLC (Method C): Rt=11.82 min, 98.89%.

### Step 5. (±)-2-((4-(6-((6-Benzyl-5-ethyl-5,6,7,8-tetrahydro-1,6-naphthyridine-4-yl)oxy)-2-azaspiro[3.3]heptan-2-yl)pyrimidin-5-yl)oxy)-5-fluorobenzoic Acid

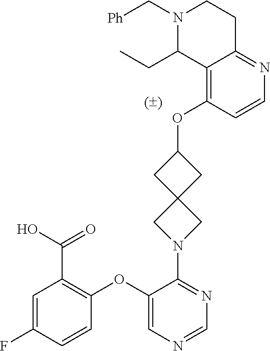 [SMILES: CCC1c2c(OC3CC4(C3)CN(c3ncncc3Oc3ccc(F)cc3C(=O)O)C4)ccnc2CCN1Cc1ccccc1]

This compound was synthesized following the procedure described for the synthesis of Intermediate 45, step 5, starting with 1 equivalent of (±)-methyl 2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluorobenzoate. After completion, the reaction was concentrated under reduced pressure. The residue was acidified with 1.5N HCl (pH˜7). The resulting solid was filtered and dried under vacuum to afford crude (±)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluorobenzoic acid as a solid. This material was used without further purification.

Yield: 97%; ¹H NMR (400 MHz, DMSO-d₆): δ 8.23 (s, 1H), 8.19 (d, J=5.7 Hz, 1H), 7.58-7.48 (m, 2H), 7.41-7.21 (m, 6H), 7.06 (dd, J=4.5, 8.9 Hz, 1H), 6.69 (d, J=5.7 Hz, 1H), 4.74 (quin, J=6.4 Hz, 1H), 4.29 (br s, 2H), 4.20 (br s, 2H), 3.76-3.47 (m, 3H), 3.22-3.11 (m, 1H), 2.97-2.84 (m, 1H), 2.83-2.70 (m, 3H), 2.48-2.44 (m, 1H), 2.26-2.12 (m, 2H), 1.68-1.47 (m, 2H), 0.91 (t, J=7.2 Hz, 3H); one proton merged with solvent peaks; LCMS (Method A): Rt=1.51 min, m/z=596.2 [M+H]⁺, 97.96%.

### Intermediate 47. 2-((4-(6-((6-(tert-Butoxycarbonyl)-6,7-dihydro-5H-pyrrolo[3,4-b]pyridin-4-yl)oxy)-2-azaspiro[3.3]heptan-2-yl)pyrimidin-5-yl)oxy)-5-fluorobenzoic Acid

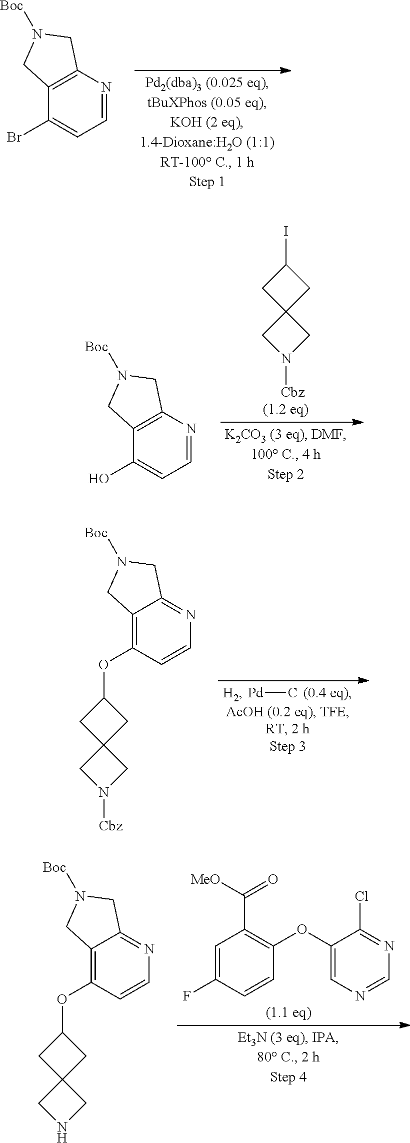

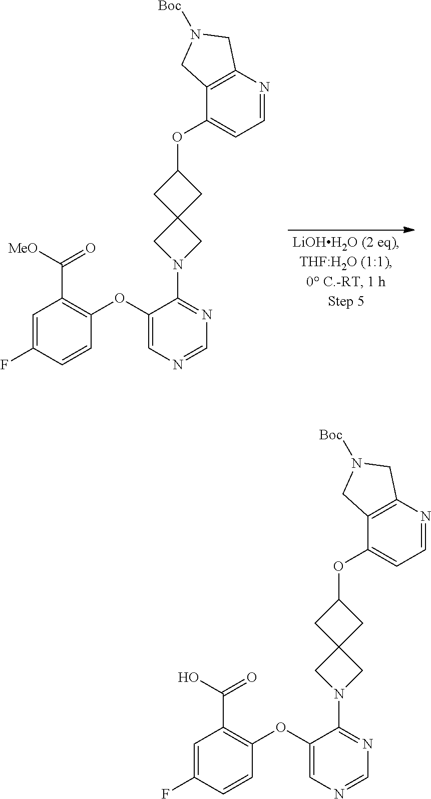 [SMILES: COC(=O)c1cc(F)ccc1Oc1cncnc1N1CC2(CC(Oc3ccnc4c3CN(C(=O)OC(C)(C)C)C4)C2)C1 ; CC(C)(C)OC(=O)N1Cc2nccc(OC3CC4(C3)CN(c3ncncc3Oc3ccc(F)cc3C(=O)O)C4)c2C1]

### Step 1. tert-Butyl 4-hydroxy-5,7-dihydro-6H-pyrrolo[3,4-b]pyridine-6-carboxylate

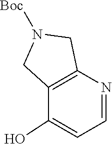

Nitrogen was purged through a solution of tert-butyl 4-bromo-5,7-dihydro-6H-pyrrolo[3,4-b]pyridine-6-carboxylate (3.9 g, 13.04 mmol) and KOH (1.463 g, 26.1 mmol) in 1,4-dioxane (30 mL) and water (30 mL) for 5 min. To this solution, tBuXPhos (0.277 g, 0.652 mmol) and Pd₂(dba)₃ (0.298 g, 0.326 mmol) were added at RT. The reaction was stirred at 100° C. for 1 h and monitored by TLC (10% MeOH in DCM). The reaction was concentrated under reduced pressure to remove 1,4-dioxane. The resulting mixture was diluted with water (20 mL) and washed with EtOAc in hexane (2×50 mL). The aqueous layer was neutralized with saturated ammonium chloride solution (40 mL) and the mixture was concentrated under reduced pressure. To this residue, 100% MeOH in DCM (100 mL) was added, and the solid was filtered and washed with 10% MeOH in DCM (2×100 mL). The filtrate was concentrated under reduced pressure to obtain crude tert-butyl 4-hydroxy-5,7-dihydro-6H-pyrrolo[3,4-b]pyridine-6-carboxylate (3.1 g, 93% yield) as a solid. This material was used without further purification. ¹H NMR (400 MHz, DMSO-d₆): δ 8.01-7.75 (m, 1H), 6.58-6.10 (m, 1H), 4.55-4.46 (m, 2H), 4.43-4.28 (m, 2H), 1.45 (s, 9H), one proton merged with solvent peaks; LCMS (Method A). Rt=1.18 min, m/z=237.0 [M+H]⁺, 92.14%.

### Step 2. tert-Butyl 4-((2-((benzyloxy)carbonyl)-2-azaspiro[3.3]heptan-6-yl)oxy)-5,7-dihydro-6H-pyrrolo[3,4-b]pyridine-6-carboxylate

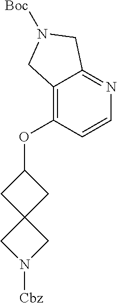 [SMILES: CC(C)(C)OC(=O)N1Cc2nccc(OC3CC4(C3)CN(C(=O)OCc3ccccc3)C4)c2C1]

This compound was synthesized following the procedure described for the synthesis of Intermediate 16, step 2, starting with 1 equivalent of 6-iodo-2-azaspiro[3.3]heptane-2-carboxylate and 1.2 equivalents of tert-butyl 4-hydroxy-5,7-dihydro-6H-pyrrolo[3,4-b]pyridine-6-carboxylate. After work-up, the crude product was purified by silica gel flash column chromatography using MeOH in DCM (product eluted at 6% MeOH in DCM) to obtain tert-butyl 4-((2-((benzyloxy)carbonyl)-2-azaspiro[3.3]heptan-6-yl)oxy)-5,7-dihydro-6H-pyrrolo[3,4-b]pyridine-6-carboxylate as a liquid.

Yield: 58.4%; ¹H NMR (400 MHz, DMSO-d₆): δ 8.32 (d, J=5.8 Hz, 1H), 7.44-7.27 (m, 5H), 6.80 (d, J=5.8 Hz, 1H), 5.03 (s, 2H), 4.80 (quin, J=6.7 Hz, 1H), 4.56-4.44 (m, 4H), 4.11-3.89 (m, 4H), 2.84-2.72 (m, 2H), 2.31-2.21 (m, 2H), 1.46 (s, 9H); LCMS (Method A): Rt=2.13 min, m/z=466.2 [M+H]⁺, 95.88%.

### Step 3. tert-Butyl 4-((2-azaspiro[3.3]heptan-6-yl)oxy)-5,7-dihydro-6H-pyrrolo[3,4-b]pyridine-6-carboxylate

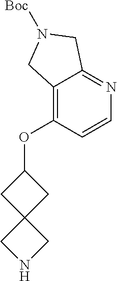 [SMILES: CC(C)(C)OC(=O)N1Cc2nccc(OC3CC4(CNC4)C3)c2C1]

In a 25 mL round bottom flask, tert-butyl 4-((2-((benzyloxy)carbonyl)-2-azaspiro[3.3]heptan-6-yl)oxy)-5,7-dihydro-6H-pyrrolo[3,4-b]pyridine-6-carboxylate (1.0 g, 2.148 mmol) was dissolved in TFE (10 mL). To this solution, AcOH (0.025 ml, 0.430 mmol) and Pd—C (0.914 g, 8.59 mmol) were added, and the reaction was stirred at RT for 2 h under a hydrogen atmosphere (balloon pressure). The reaction progress was monitored by TLC (10% MeOH in DCM). After completion, the reaction was filtered through a Celite® pad, and the pad was washed with EtOAc:THF (150 mL, 1:1). The filtrate was concentrated under reduced pressure and the residue was triturated with 10% EtOAc in hexane (2×10 mL). The organic layer was decanted, and the resulting solid was dried under vacuum to afford crude tert-butyl 4-((2-azaspiro[3.3]heptan-6-yl)oxy)-5,7-dihydro-6H-pyrrolo[3,4-b]pyridine-6-carboxylate (0.58 g, 81% yield) as a solid. This material was used without further purification. ¹H NMR (400 MHz, DMSO-d₆): δ 8.31 (d, J=5.7 Hz, 1H), 6.80 (d, J=5.7 Hz, 1H), 4.76 (quin, J=6.7 Hz, 1H), 4.57-4.44 (m, 4H), 3.65 (br s, 2H), 3.57 (br s, 2H), 2.81-2.68 (m, 2H), 2.23-2.11 (m, 2H), 1.46 (s, 9H), one proton merged with solvent peaks; LCMS (Method A): Rt=1.37 min, m/z=332.1 [M+H]⁺, 99.65%.

### Step 4. tert-Butyl 4-((2-(5-(4-fluoro-2-(methoxycarbonyl)phenoxy)pyrimidin-4-yl)-2-azaspiro[3.3]heptan-6-yl)oxy)-5,7-dihydro-6H-pyrrolo[3,4-b]pyridine-6-carboxylate

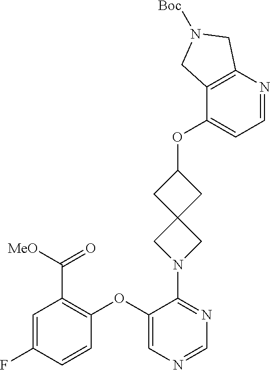 [SMILES: COC(=O)c1cc(F)ccc1Oc1cncnc1N1CC2(CC(Oc3ccnc4c3CN(C(=O)OC(C)(C)C)C4)C2)C1]

In a 40 mL pressure vial, tert-butyl 4-((2-azaspiro[3.3]heptan-6-yl)oxy)-5,7-dihydro-6H-pyrrolo[3,4-b]pyridine-6-carboxylate (0.53 g, 1.599 mmol) and methyl 2-((4-chloropyrimidin-5-yl)oxy)-5-fluorobenzoate (0.497 g, 1.759 mmol) were dissolved in IPA (10 mL). To this reaction mixture, TEA (0.669 mL, 4.80 mmol) was added at RT and the reaction was stirred at 80° C. for 2 h. The reaction progress was monitored by TLC (10% MeOH in DCM). After completion, the reaction was concentrated under reduced pressure. Water (50 mL) was added to the residue and the mixture was extracted with EtOAc (2×50 mL). The combined organic layer was washed with brine solution (25 mL), dried over anhydrous sodium sulfate and filtered, and the filtrate was concentrated under reduced pressure. The crude product was purified by silica gel flash column chromatography using MeOH in DCM (product eluted at 4% MeOH in DCM) to obtain tert-butyl 4-((2-(5-(4-fluoro-2-(methoxycarbonyl)phenoxy)pyrimidin-4-yl)-2-azaspiro[3.3]heptan-6-yl)oxy)-5,7-dihydro-6H-pyrrolo[3,4-b]pyridine-6-carboxylate (0.605 g, 65.4% yield) as a solid. ¹H NMR (400 MHz, DMSO-d₆): δ 8.38-8.27 (m, 2H), 7.75-7.63 (m, 2H), 7.55-7.44 (m, 1H), 7.18-7.05 (m, 1H), 6.81 (d, J=5.8 Hz, 1H), 4.83 (quin, J=6.7 Hz, 1H), 4.58-4.44 (m, 4H), 4.27 (s, 2H), 4.19 (s, 2H), 3.82 (s, 3H), 2.88-2.75 (m, 2H), 2.38-2.24 (m, 2H), 1.46 (s, 9H); LCMS (Method A): Rt=2.04 min, m/z=578.1 [M+H]⁺, 99.84%.

### Step 5. 2-((4-(6-((6-(tert-Butoxycarbonyl)-6,7-dihydro-5H-pyrrolo[3,4-b]pyridin-4-yl)oxy)-2-azaspiro[3.3]heptan-2-yl)pyrimidin-5-yl)oxy)-5-fluorobenzoic Acid

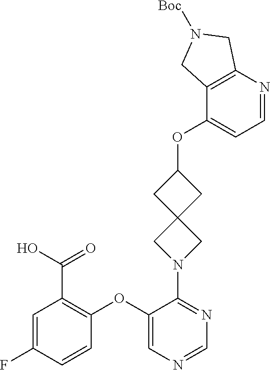 [SMILES: CC(C)(C)OC(=O)N1Cc2nccc(OC3CC4(C3)CN(c3ncncc3Oc3ccc(F)cc3C(=O)O)C4)c2C1]

In a dried, 100 mL round bottom flask under a nitrogen atmosphere, tert-butyl 4-((2-(5-(4-fluoro-2-(methoxycarbonyl)phenoxy)pyrimidin-4-yl)-2-azaspiro[3.3]heptan-6-yl)oxy)-5,7-dihydro-6H-pyrrolo[3,4-b]pyridine-6-carboxylate (1.62 g, 2.80 mmol) was dissolved in THF (10 mL). To this reaction mixture, a solution of LiOH·H₂O (0.235 g, 5.61 mmol) in H₂O (10 mL) was added at 0° C., and the reaction was stirred at RT for 1 h. The reaction progress was monitored by TLC (10% MeOH in DCM). After completion, the reaction was concentrated under reduced pressure. The residue was diluted with water (4 mL) and acidified with 1.5N HCl (pH˜6). The mixture was extracted with 10% MeOH in DCM (3×15 mL). The combined organic layer was dried over anhydrous sodium sulfate and filtered, and the filtrate was concentrated under reduced pressure to obtain crude 2-((4-(6-((6-(tert-butoxycarbonyl)-6,7-dihydro-5H-pyrrolo[3,4-b]pyridin-4-yl)oxy)-2-azaspiro[3.3]heptan-2-yl)pyrimidin-5-yl)oxy)-5-fluorobenzoic acid (1.49 g, 91% yield) as a solid. This material was used without further purification. LCMS (Method B): Rt=1.48 min, m/z=564.4 [M+H]⁺, 97.02%.

### Intermediate 48. 5-Fluoro-N-isopropyl-N-((1r,3r)-3-methyl-3-(methylamino)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl

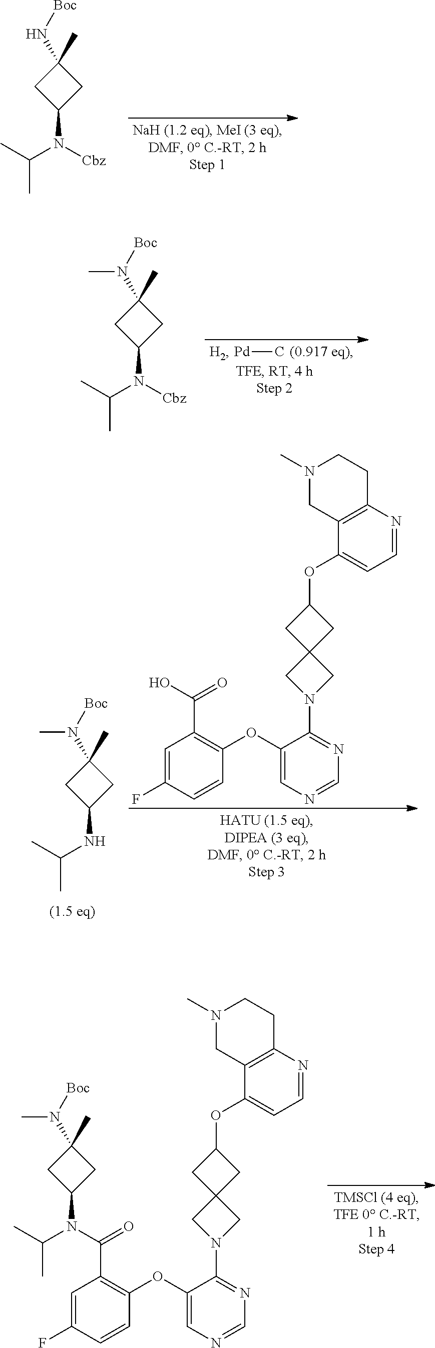

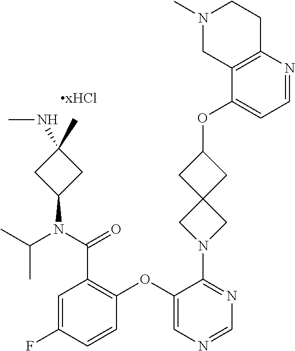 [SMILES: CN[C@]1(C)C[C@@H](N(C(=O)c2cc(F)ccc2Oc2cncnc2N2CC3(CC(Oc4ccnc5c4CN(C)CC5)C3)C2)C(C)C)C1]

### Step 1. tert-Butyl ((1r,3r)-3-(((benzyloxy)carbonyl)(isopropyl)amino)-1-methylcyclobutyl)(methyl)carbamate

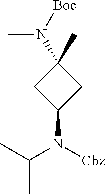 [SMILES: CC(C)N(C(=O)OCc1ccccc1)[C@H]1C[C@](C)(N(C)C(=O)OC(C)(C)C)C1]

In a dried, 50 mL two-neck round bottom flask under a nitrogen atmosphere, benzyl ((1r,3r)-3-((tert-butoxycarbonyl)amino)-3-methylcyclobutyl)(isopropyl)carbamate (310 mg, 0.823 mmol) was dissolved in DMF (11 mL). To this solution, sodium hydride (39.5 mg, 0.988 mmol) was added at 0° C., and the reaction was stirred at the same temperature for 15 min. MeI (0.154 mL, 2.470 mmol) was added to this reaction mixture at 0° C. The reaction was stirred at RT for 2 h. After completion, the reaction was quenched with aqueous ammonium chloride (10 mL) and the mixture was extracted with EtOAc (3×10 mL). The combined organic layer was washed with brine (30 mL), dried over anhydrous sodium sulfate and filtered, and the filtrate was concentrated under reduced pressure. The crude compound was purified by silica gel flash column chromatography using EtOAc in hexane (product eluted at 10% EtOAc in hexane) to obtain benzyl ((1r,3r)-3-((tert-butoxycarbonyl)(methyl)amino)-3-methylcyclobutyl)(isopropyl)carbamate (360 mg, 89% yield) as a sticky liquid. ¹H NMR (400 MHz, DMSO-d₆): δ 7.40-7.30 (m, 5H), 5.11-5.06 (m, 2H), 4.05-3.88 (m, 2H), 2.74 (s, 3H), 2.64-2.56 (m, 2H), 2.36-2.26 (m, 2H), 1.38 (s, 9H), 1.22 (s, 3H), 1.12 (d, J=6.9 Hz, 6H); LCMS (Method A): Rt=2.50 min, m/z=391.1 [M+H]⁺, 79.77%.

### Step 2. tert-Butyl ((1r,3r)-3-(isopropylamino)-1-methylcyclobutyl)(methyl)carbamate

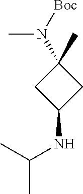 [SMILES: CC(C)N[C@H]1C[C@](C)(N(C)C(=O)OC(C)(C)C)C1]

This compound was synthesized following the procedure described for the synthesis of Intermediate 22, step 2, starting with 1 equivalent of benzyl ((1r,3r)-3-((tert-butoxycarbonyl)(methyl)amino)-3-methylcyclobutyl)(isopropyl)carbamate, except that 0.917 equivalent of Pd—C was used. After filtration, the filtrate was concentrated under reduced pressure to afford crude tert-butyl ((1r,3r)-3-(isopropylamino)-1-methylcyclobutyl)(methyl)carbamate as a liquid. This material was used without further purification.

Yield: 39.9%; ¹H NMR (400 MHz, DMSO-d₆): δ 3.19-3.12 (m, 1H), 2.70-2.64 (m, 1H), 2.62 (s, 3H), 2.48-2.41 (m, 2H), 1.69-1.62 (m, 2H), 1.41-1.28 (m, 12H), 0.93 (d, J=6.3 Hz, 6H), one proton merged with solvent peaks.

### Step 3. tert-Butyl ((1r,3r)-3-(5-fluoro-N-isopropyl-2-((4-((6-methyl-5,6,7,8-tetrahydro-1,6-naphthyridine-4-yl)oxy)-2-azaspiro[3.3]heptan-2-yl)pyrimidin-5-yl)oxy)benzamido)-1-methylcyclobutyl)(methyl)carbamate

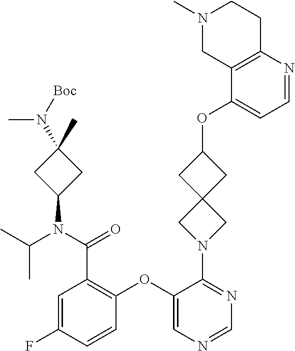 [SMILES: CC(C)N(C(=O)c1cc(F)ccc1Oc1cncnc1N1CC2(CC(Oc3ccnc4c3CN(C)CC4)C2)C1)[C@H]1C[C@](C)(N(C)C(=O)OC(C)(C)C)C1]

This compound was synthesized following the procedure described for the synthesis of Intermediate 26, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.5 equivalents of tert-butyl ((1r,3r)-3-(isopropylamino)-1-methylcyclobutyl)(methyl)carbamate. After work-up, the crude was purified by reverse phase chromatography using a SiliCycle SiliaSep C18 column (80 g) on Biotage isolera one (Mobile phase A: 10 mM NH₄HCO₃ in water, B: ACN). The fractions were concentrated under reduced pressure and lyophilized to afford tert-butyl ((1r,3r)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-1-methylcyclobutyl)(methyl)carbamate as a solid.

Yield: 84%; ¹H NMR (400 MHz, DMSO-d₆): δ 8.31-8.23 (m, 1H), 8.18 (br d, J=5.5 Hz, 11H), 7.79-7.58 (m, 1H), 7.33-7.05 (m, 3H), 6.69-6.62 (m, 1H), 4.75 (quin, J=6.6 Hz, 1H), 4.37-4.09 (m, 4H), 3.94-3.81 (m, 1H), 3.76-3.64 (m, 1H), 3.36 (s, 2H), 3.20-2.94 (m, 1H), 2.85-2.73 (m, 6H), 2.66-2.58 (m, 4H), 2.36 (s, 3H), 2.30-2.21 (m, 2H), 2.02-1.89 (m, 1H), 1.47-1.30 (m, 12H), 1.28-0.92 (m, 6H), one proton merged with solvent peaks; LCMS (Method A): Rt=2.18 min, m/z=730.6 [M+H]⁺, 86.94%.

### Step 4. 5-Fluoro-N-isopropyl-N-((1r,3r)-3-methyl-3-(methylamino)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl

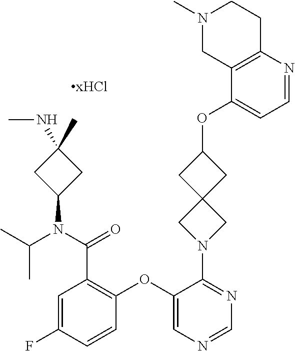 [SMILES: CN[C@]1(C)C[C@@H](N(C(=O)c2cc(F)ccc2Oc2cncnc2N2CC3(CC(Oc4ccnc5c4CN(C)CC5)C3)C2)C(C)C)C1]

This compound was synthesized following the procedure described for the synthesis of Intermediate 16, step 3, starting with 1 equivalent of tert-butyl ((1r,3r)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-1-methylcyclobutyl)(methyl)carbamate. After completion, the reaction was concentrated under reduced pressure. The crude was reconcentrated from EtOAc, and the residue was dried under reduced pressure to obtain crude 5-fluoro-N-isopropyl-N-((1r,3r)-3-methyl-3-(methylamino)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl as a solid. This material was used without further purification.

Yield: Quantitative; LCMS (Method A): Rt=1.74 min, m/z=630.5 [M+H]⁺, 87.19%.

### Intermediate 49. tert-Butyl 8-((2-azaspiro[3.3]heptan-6-yl)oxy)-3,4-dihydroisoquinoline-2(1H)-carboxylate

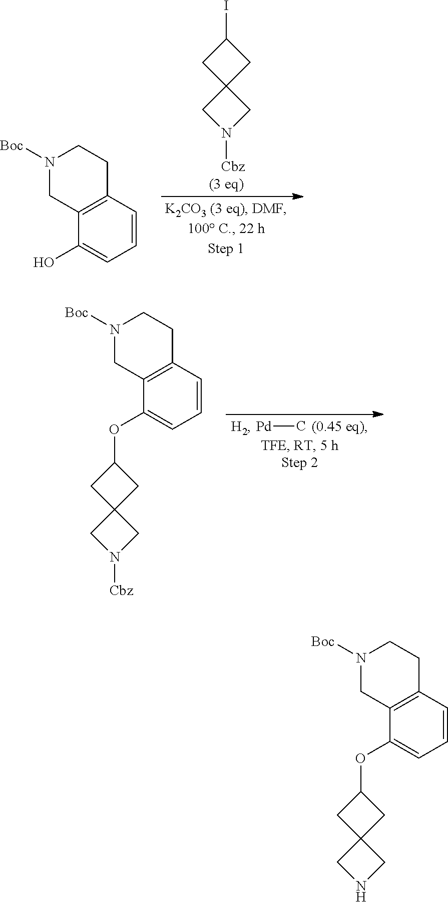

### Step 1. tert-Butyl 8-((2-((benzyloxy)carbonyl)-2-azaspiro[3.3]heptan-6-yl)oxy)-3,4-dihydroisoquinoline-2(1H)-carboxylate

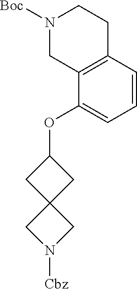 [SMILES: CC(C)(C)OC(=O)N1CCc2cccc(OC3CC4(C3)CN(C(=O)OCc3ccccc3)C4)c2C1]

This compound was synthesized following the procedure described for the synthesis of Intermediate 24, step 1, starting with 1 equivalent of tert-butyl 8-hydroxy-3,4-dihydroisoquinoline-2(1H)-carboxylate and 3 equivalents of benzyl 6-iodo-2-azaspiro[3.3]heptane-2-carboxylate. The reaction was diluted with water and the mixture was extracted with EtOAc. The combined organic layer was washed with brine, dried over anhydrous sodium sulfate and filtered, and the filtrate was concentrated under reduced pressure. The crude product was purified by silica gel flash column chromatography using MeOH in DCM (product eluted at 12% MeOH in DCM) to obtain tert-butyl 8-((2-((benzyloxy)carbonyl)-2-azaspiro[3.3]heptan-6-yl)oxy)-3,4-dihydroisoquinoline-2(1H)-carboxylate as a gummy solid.

Yield: 82%; ¹H NMR (400 MHz, DMSO-d₆): δ 7.40-7.29 (m, 5H), 7.10 (t, J=7.9 Hz, 1H), 6.76-6.71 (m, 1H), 6.64 (d, J=8.1 Hz, 1H), 5.02 (s, 2H), 4.64 (quin, J=6.5 Hz, 1H), 4.35 (s, 2H), 4.07-3.89 (m, 4H), 3.52 (br t, J=5.6 Hz, 2H), 2.77-2.69 (m, 4H), 2.22 (br s, 2H), 1.42 (s, 9H); LCMS (Method B): Rt=2.94-2.96 min, m/z=423.4 [M+H−56]⁺, 94.31%.

### Step 2. tert-Butyl 8-((2-azaspiro[3.3]heptan-6-yl)oxy)-3,4-dihydroisoquinoline-2(1H)-carboxylate

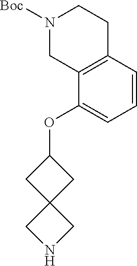 [SMILES: CC(C)(C)OC(=O)N1CCc2cccc(OC3CC4(CNC4)C3)c2C1]

This compound was synthesized following the procedure described for the synthesis of Intermediate 24, step 2, starting with 1 equivalent of tert-butyl 8-((2-((benzyloxy)carbonyl)-2-azaspiro[3.3]heptan-6-yl)oxy)-3,4-dihydroisoquinoline-2(1H)-carboxylate, except that 0.45 equivalent of Pd—C was used. After completion, the filtrate was concentrated under reduced pressure to obtain crude tert-butyl 8-((2-azaspiro[3.3]heptan-6-yl)oxy)-3,4-dihydroisoquinoline-2(1H)-carboxylate as a liquid. This material was used without further purification.

Yield: Quantitative; ¹HNMR (400 MHz, DMSO-d₆): δ 7.10 (t, J=7.9 Hz, 1H), 6.73 (br d, J=7.5 Hz, 1H), 6.64 (br d, J=8.0 Hz, 1H), 4.59 (quin, 1=6.5 Hz, 1H), 4.34 (br s, 2H), 3.63-3.49 (m, 5H), 2.76-2.65 (m, 4H), 2.16-2.09 (m, 2H), 1.43 (s, 9H), two protons merged with solvent peaks; LCMS-ELSD (Method A): Rt=1.80 min, m/z=345.1 [M+H]⁺, 95.71%.

### Intermediate 50. N-((1r,3r)-3-(Azetidin-1-yl)-3-methylcyclobutyl)-5-fluoro-2-hydroxy-N-isopropylbenzamide

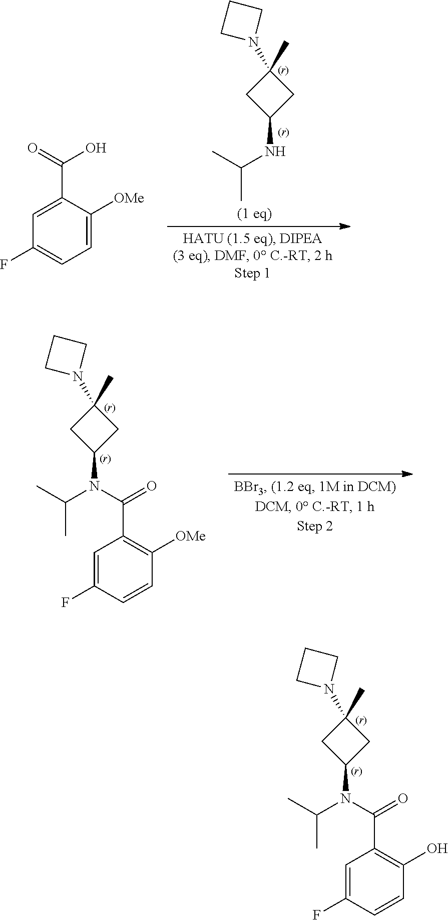 [SMILES: COc1ccc(F)cc1C(=O)O ; CC(C)N[C@H]1C[C@](C)(N2CCC2)C1 ; COc1ccc(F)cc1C(=O)N(C(C)C)[C@H]1C[C@](C)(N2CCC2)C1 ; CC(C)N(C(=O)c1cc(F)ccc1O)[C@H]1C[C@](C)(N2CCC2)C1]

### Step 1. N-((1r,3r)-3-(Azetidin-1-yl)-3-methylcyclobutyl)-5-fluoro-N-isopropyl-2-methoxybenzamide

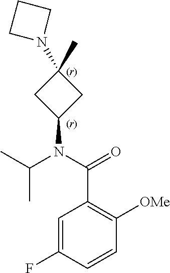 [SMILES: COc1ccc(F)cc1C(=O)N(C(C)C)[C@H]1C[C@](C)(N2CCC2)C1]

This compound was synthesized following the procedure described for the synthesis of Intermediate 43, Step 1, starting with 1 equivalent of 5-fluoro-2-methoxybenzoic acid and 1 equivalent of (1r,3r)-3-(azetidin-1-yl)-N-isopropyl-3-methylcyclobutan-1-amine. After work-up, the crude was purified by reverse phase column using a SiliCycle SiliaSep C18 column (120 g) on Biotage isolera one (Mobile phase A: 10 mm NH₄HCO₃ in water, B: ACN) to obtain N-((1r,3r)-3-(azetidin-1-yl)-3-methylcyclobutyl)-5-fluoro-N-isopropyl-2-methoxybenzamide as a solid.

Yield: 31.1%; LCMS (Method A): Rt=1.63 min, m/z=335.3 [M+H]⁺; HPLC (Method C): Rt=8.25 min, 95.07%.

### Step 2. N-((1r,3r)-3-(Azetidin-1-yl)-3-methylcyclobutyl)-5-fluoro-2-hydroxy-N-isopropylbenzamide

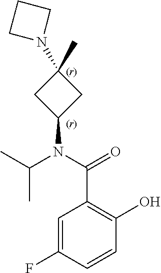 [SMILES: CC(C)N(C(=O)c1cc(F)ccc1O)[C@H]1C[C@](C)(N2CCC2)C1]

This compound was synthesized following the procedure described for the synthesis of Intermediate 43, Step 2, starting with 1 equivalent of N-((1r,3r)-3-(azetidin-1-yl)-3-methylcyclobutyl)-5-fluoro-N-isopropyl-2-methoxybenzamide, except that 1.2 equivalents of BBr₃ (IM in DCM) was used. After completion, the reaction was quenched with methanol and re-concentrated from MeOH 4 times. To this residue, aqueous NaHCO₃ solution was added and the mixture was extracted with 10% MeOH in DCM. The combined organic layer was dried over sodium sulfate and filtered, and the filtrate was concentrated under reduced pressure to obtain crude N-((1r,3r)-3-(azetidin-1-yl)-3-methylcyclobutyl)-5-fluoro-2-hydroxy-N-isopropylbenzamide as a solid. This material was used without further purification.

Yield: 68.5%; ¹H NMR (400 MHz, DMSO-d₆): δ 9.67 (br s, 1H), 7.03 (dt, J=3.1, 8.7 Hz, 1H), 6.96-6.89 (m, 1H), 6.85 (dd, J=4.4, 8.9 Hz, 1H), 4.04-3.89 (m, 1H), 3.74-3.55 (m, 3H), 2.95-2.72 (m, 1H), 2.41-1.98 (m, 4H), 1.50-1.01 (m, 9H), three protons merged with solvent peaks; LCMS (Method A): Rt=1.58 min, m/z=321.1 [M+H]⁺; HPLC (Method C): Rt=7.46 min, 86.53%.

### Intermediate 51. tert-Butyl 2-(isopropylamino)-5-azaspiro[3.5]nonane-5-carboxylate

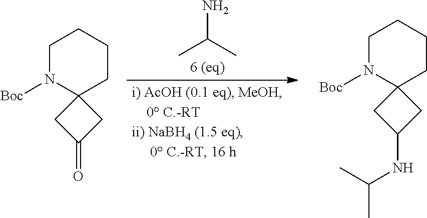

In a 40 mL pressure vial, tert-butyl 2-oxo-5-azaspiro[3.5]nonane-5-carboxylate (1.0 g, 4.18 mmol) was dissolved in MeOH (3 mL). To this stirred solution, propan-2-amine (2.148 mL, 25.07 mmol) and AcOH (0.024 mL, 0.418 mmol) were added at 0° C. The reaction was stirred at RT. After 16 h, NaBH₄ (0.237 g, 6.27 mmol) was added at 0° C., and the reaction was continued at 0° C. for 1 h. The reaction progress was monitored by TLC (30% EtOAc in hexane). After completion, the reaction was concentrated under reduced pressure. The residue was diluted with water (30 mL) and the mixture was extracted with EtOAc (3×45 mL). The combined organic layer was dried over sodium sulfate and filtered, and the filtrate was concentrated under reduced pressure to obtain crude tert-butyl 2-(isopropylamino)-5-azaspiro[3.5]nonane-5-carboxylate (1.1 g, 93% yield) as a gummy liquid. This material was used without further purification. ¹H NMR (400 MHz, DMSO-d₆): δ 3.21-3.07 (m, 3H), 2.92-2.60 (m, 2H), 2.48-2.32 (m, 2H), 1.83-1.71 (m, 3H), 1.65-1.49 (m, 3H), 1.39 (s, 9H), 1.33-1.21 (m, 2H), 0.93 (dd, J=2.1, 6.2 Hz, 6H); LCMS (Method B): Rt=1.40 min, m/z=283.2 [M+H]⁺, 99.90%.

### Intermediate 52. 1-Chloroethyl (4-nitrophenyl) carbonate

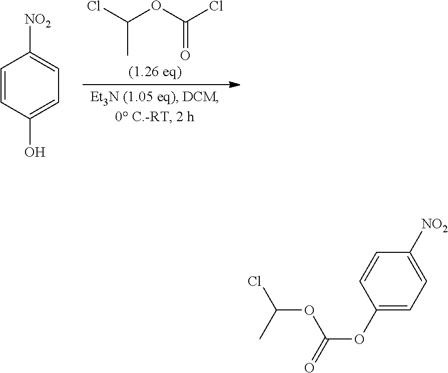

To a stirred solution of 4-nitrophenol (10 g, 71.9 mmol) in DCM (100 mL), triethylamine (10.52 mL, 75 mmol) was added at 0° C. To this reaction mixture, a solution of 1-chloroethyl carbonochloridate (9.81 mL, 91 mmol) in DCM (50 mL) was added at 0° C. The reaction was stirred at RT for 2 h. The reaction progress was monitored by TLC (10% EtOAc in hexane). After completion, the reaction was quenched with water (100 mL) and the mixture was extracted with DCM (3×150 mL). The combined organic layer was washed with 10% NaHCO₃ solution (100 mL), dried over sodium sulfate and filtered, and the filtrate was concentrated under reduced pressure to crude obtain 1-chloroethyl (4-nitrophenyl) carbonate (17 g, 96% yield) as a solid. This material was used without further purification. ¹H NMR (400 MHz, DMSO-d₆): δ 8.36-8.30 (m, 2H), 7.48-7.41 (m, 2H), 6.53 (q, J=5.8 Hz, 1H), 1.95 (d, J=5.9 Hz, 3H).

### Intermediate 53. 1-Chloro-2-methylpropyl (4-nitrophenyl) carbonate

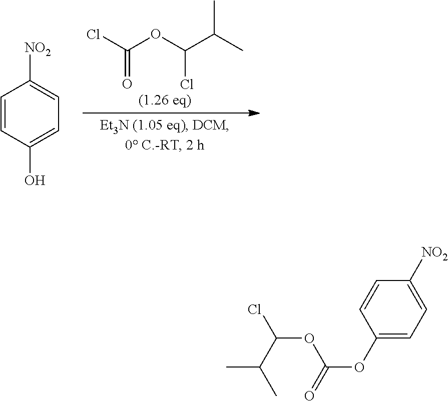

This compound was synthesized following the procedure described for the synthesis of Intermediate 52, starting with 1 equivalent of 4-nitrophenol and 1.26 equivalents of 1-chloro-2-methylpropyl carbonochloridate. After completion, the reaction was quenched with 10% NaHCO₃ solution and the mixture was extracted with DCM (3×150 mL). The combined organic layer was washed with 10% NaOH solution, dried over sodium sulfate and filtered, and the filtrate was concentrated under reduced pressure to obtain 1-chloro-2-methylpropyl (4-nitrophenyl) carbonate as a liquid (2.6 g, 83% yield) as a liquid. This material was used without further purification. ¹H NMR (400 MHz, DMSO-d₆): δ 8.38-8.32 (m, 2H), 7.67-7.61 (m, 2H), 6.45 (d, J=4.1 Hz, 1H), 2.38-2.25 (m, 1H), 1.09-1.02 (m, 6H).

### Intermediate 54. (E1)-4-((2-Azaspiro[3.3]heptan-6-yl)oxy)-6-benzyl-5-ethyl-5,6,7,8-tetrahydro-1,6-naphthyridine xHCl

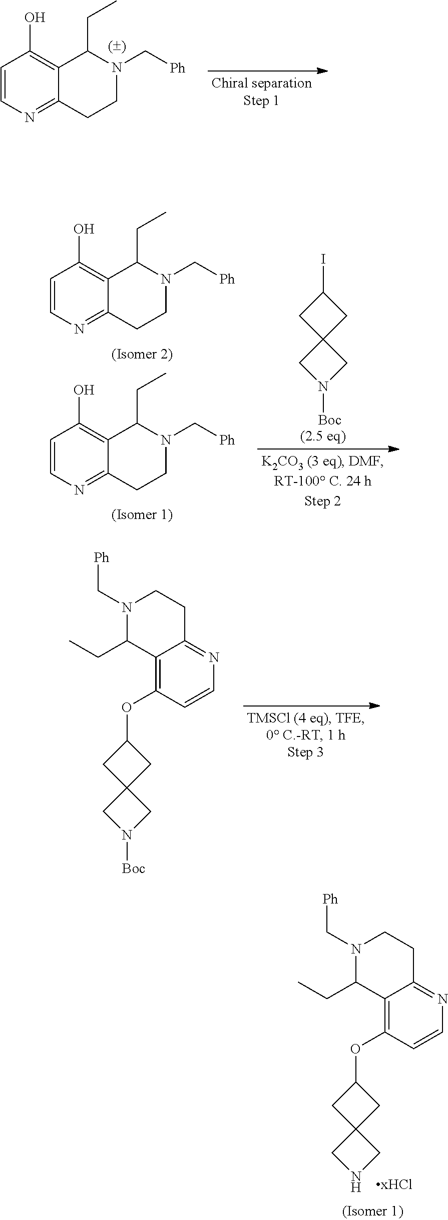 [SMILES: CCC1c2c(O)ccnc2CCN1Cc1ccccc1 ; CCC1c2c(O)ccnc2CCN1Cc1ccccc1 ; CCC1c2c(O)ccnc2CCN1Cc1ccccc1 ; CC(C)(C)OC(=O)N1CC2(CC(I)C2)C1 ; CCC1c2c(OC3CC4(C3)CN(C(=O)OC(C)(C)C)C4)ccnc2CCN1Cc1ccccc1 ; CCC1c2c(OC3CC4(CNC4)C3)ccnc2CCN1Cc1ccccc1]

### Step 1. (E1)-6-Benzyl-5-ethyl-5,6,7,8-tetrahydro-1,6-naphthyridin-4-ol and (E2)-6-benzyl-5-ethyl-5,6,7,8-tetrahydro-1,6-naphthyridin-4-ol

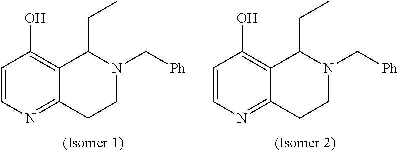

(±)-6-Benzyl-5-ethyl-5,6,7,8-tetrahydro-1,6-naphthyridin-4-ol (18 g, 67.1 mmol) was purified by chiral Prep SFC (Method AA) to afford (E1)-6-benzyl-5-ethyl-5,6,7,8-tetrahydro-1,6-naphthyridin-4-ol (6 g, 33.2% yield) and (E2)-6-benzyl-5-ethyl-5,6,7,8-tetrahydro-1,6-naphthyridin-4-ol (8 g, 43.9% yield) as solids.

Isomer 1. ¹H NMR (400 MHz, DMSO-d₆): δ 11.17 (br s, 1H), 7.49 (br d, J=7.1 Hz, 1H), 7.36-7.22 (m, 5H), 5.93 (br d, J=7.1 Hz, 1H), 3.71-3.51 (m, 2H), 3.38-3.36 (m, 1H), 3.19-3.06 (m, 1H), 2.81-2.66 (m, 2H), 2.30-2.15 (m, 1H), 1.74-1.60 (m, 1H), 1.50-1.29 (m, 1H), 0.87 (t, J=7.3 Hz, 3H); LCMS (Method A): Rt=1.46 min, m/z=269.1 [M+H]⁺; HPLC (Method C): Rt=7.83 min, 99.71%; Chiral SFC (Method AAO): Rt=4.17 min, 100%; [α]_{D}²⁵=+40.44° (c 0.733, MeOH).

Isomer 2. ¹H NMR (400 MHz, DMSO-d₆): δ 11.18 (br s, 1H), 7.48 (br d, J=6.0 Hz, 1H), 7.37-7.22 (m, 5H), 5.93 (d, J=7.1 Hz, 1H), 3.73-3.50 (m, 2H), 3.38-3.34 (m, 1H), 3.17-3.06 (m, 1H), 2.80-2.66 (m, 2H), 2.29-2.20 (m, 1H), 1.73-1.61 (m, 1H), 1.44-1.30 (m, 1H), 0.87 (t, J=7.3 Hz, 3H); LCMS (Method A): Rt=1.46 min, m/z=269.1 [M+H]⁺; HPLC (Method C): Rt=7.84 min, 98.83%; Chiral SFC (Method AAO): Rt=5.30 min, 98.40%. [α]_{D}²⁵=−29.46° (c 1.0, MeOH).

### Step 2. (E1)-tert-Butyl 6-((6-benzyl-5-ethyl-5,6,7,8-tetrahydro-1,6-naphthyridin-4-yl)oxy)-2-azaspiro[3.3]heptane-2-carboxylate

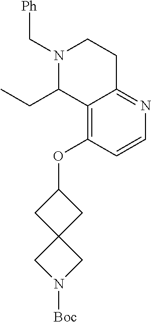 [SMILES: CCC1c2c(OC3CC4(C3)CN(C(=O)OC(C)(C)C)C4)ccnc2CCN1Cc1ccccc1]

This compound was synthesized following the procedure described for the synthesis of Intermediate 16, step 2, starting with 1 equivalent (E1)-6-benzyl-5-ethyl-5,6,7,8-tetrahydro-1,6-naphthyridin-4-ol and 2.5 equivalents of tert-butyl 6-iodo-2-azaspiro[3.3]heptane-2-carboxylate. After work-up, the crude product was purified by silica gel flash column chromatography using MeOH in DCM (product eluted at 8% MeOH in DCM) to obtain (E1)-tert-butyl (R)-6-((6-benzyl-5-ethyl-5,6,7,8-tetrahydro-1,6-naphthyridin-4-yl)oxy)-2-azaspiro[3.3]heptane-2-carboxylate as a solid.

Yield: 85%; ¹H NMR (400 MHz, DMSO-d₆): δ 8.21 (d, J=5.5 Hz, 1H), 7.37-7.29 (m, 4H), 7.28-7.23 (m, 1H), 6.67 (d, 1=5.7 Hz, 11H), 4.70 (quin, J=6.4 Hz, 1H), 3.89 (br s, 2H), 3.81 (br s, 2H), 3.70 (d, J=13.4 Hz, 1H), 3.61-3.50 (m, 2H), 3.24-3.13 (m, 1H), 2.99-2.86 (m, 1H), 2.84-2.66 (m, 3H), 2.20-2.06 (m, 2H), 1.64-1.49 (m, 2H), 1.37 (s, 9H), 0.92 (t, J=7.2 Hz, 3H), one proton merged with solvent peaks; LCMS (Method A): Rt=2.42 min, m/z=464.7 [M+H]⁺, 96.53%; Chiral SFC (Method I): Rt=2.30 min, 92.70%.

### Step 3. (E1)-4-((2-Azaspiro[3.3]heptan-6-yl)oxy)-6-benzyl-5-ethyl-5,6,7,8-tetrahydro-1,6-naphthyridine xHCl

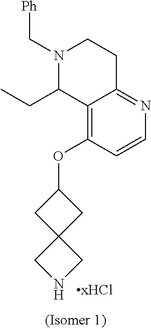 [SMILES: CCC1c2c(OC3CC4(CNC4)C3)ccnc2CCN1Cc1ccccc1]

This compound was synthesized following the procedure described for the synthesis of Intermediate 16, step 3, starting with 1 equivalent of (E1)-tert-butyl-6-((6-benzyl-5-ethyl-5,6,7,8-tetrahydro-1,6-naphthyridin-4-yl)oxy)-2-azaspiro[3.3]heptane-2-carboxylate. After completion, the reaction was concentrated under reduced pressure to obtain crude (E1)-4-((2-azaspiro[3.3]heptan-6-yl)oxy)-6-benzyl-5-ethyl-5,6,7,8-tetrahydro-1,6-naphthyridine xHCl as a sticky solid. This material was used without further purification.

Yield: Quantitative; LCMS (Method H): Rt=2.23-2.29 min, m/z=364.2 [M+H]⁺, 95.45%;

### Intermediate 55. (E1)-6-Benzyl-4-((2-(3,6-dichloro-1,2,4-triazin-5-yl)-2-azaspiro[3.3]heptan-6-yl)oxy)-5-ethyl-5,6,7,8-tetrahydro-1,6-naphthyridine

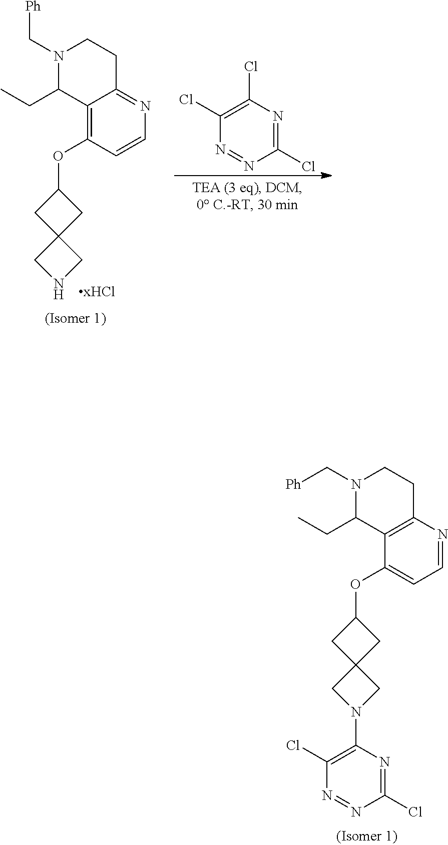

This compound was synthesized following the procedure described for the synthesis of Intermediate 34, starting with 1 equivalent of (E1)-4-((2-azaspiro[3.3]heptan-6-yl)oxy)-6-benzyl-5-ethyl-5,6,7,8-tetrahydro-1,6-naphthyridine xHCl and 1 equivalent of 3,5,6-trichloro-1,2,4-triazine. After work-up, the crude was purified by reverse phase chromatography using a SiliCycle SiliaSep C18 column (120 g) on Biotage isolera one (Mobile phase A: 10 mM NH₄HCO₃ in water, B: ACN). The pure fractions were concentrated under reduced pressure to obtain (E1)-6-benzyl-4-((2-(3,6-dichloro-1,2,4-triazin-5-yl)-2-azaspiro[3.3]heptan-6-yl)oxy)-5-ethyl-5,6,7,8-tetrahydro-1,6-naphthyridine as a solid.

Yield: 66.4%; ¹H NMR (400 MHz, DMSO-d₆): δ 8.23 (d, J=5.7 Hz, 1H), 7.37-7.20 (m, 5H), 6.70 (d, J=5.7 Hz, 1H), 4.84-4.66 (m, 3H), 4.27-4.13 (m, 2H), 3.76-3.65 (m, 1H), 3.63-3.52 (m, 2H), 3.25-3.14 (m, 1H), 2.99-2.75 (m, 4H), 2.32-2.18 (m, 2H), 1.73-1.52 (m, 2H), 1.01-0.85 (m, 3H), one proton merged with solvent peaks; LCMS (Method A): Rt=2.30 min, m/z=511.2 [M+H]⁺, 99.11%.

### Intermediate 56. (±)-1-((2-(3,6-Dichloro-1,2,4-triazin-5-yl)-2-azaspiro[3.3]heptan-6-yl)oxy)-5,6,8,9,10,10a-hexahydropyrrolo[2,1-f][1,6]naphthyridine

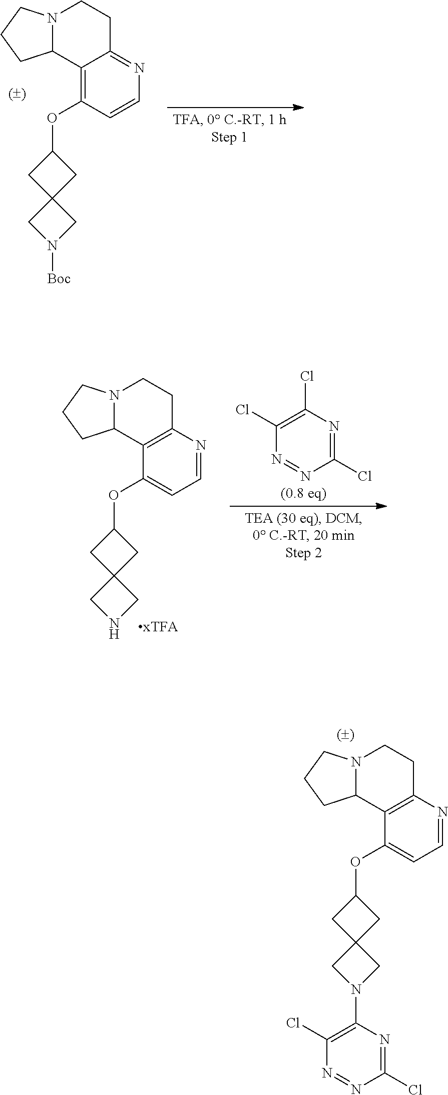 [SMILES: CC(C)(C)OC(=O)N1CC2(CC(Oc3ccnc4c3C3CCCN3CC4)C2)C1 ; c1cc(OC2CC3(CNC3)C2)c2c(n1)CCN1CCCC21 ; Clc1nnc(Cl)c(Cl)n1 ; Clc1nnc(Cl)c(N2CC3(CC(Oc4ccnc5c4C4CCCN4CC5)C3)C2)n1]

### Step 1. (±)-1-((2-Azaspiro[3.3]heptan-6-yl)oxy)-5,6,8,9,10,10a-hexahydropyrrolo[2,1-f][1,6]naphthridine xTFA

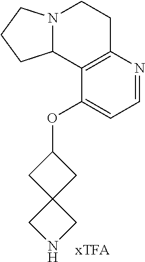 [SMILES: c1cc(OC2CC3(CNC3)C2)c2c(n1)CCN1CCCC21]

In a 250 mL two-necked round bottom flask, (f)-tert-butyl 6-((5,6,8,9,10,10α-hexahydropyrrolo[2,1-f][1,6]naphthyridin-1-yl)oxy)-2-azaspiro[3.3]heptane-2-carboxylate (5.2 g, 13.49 mmol) was dissolved trifluoroacetic acid (15 mL). The reaction was stirred at RT for 1 h. The reaction was monitored by LCMS and TLC (10% MeOH in DCM). After completion, the reaction was concentrated under reduced pressure and the residue was triturated with EtOAc (2×10 mL). The organic layer was decanted and the residue was dried under vacuum to obtain crude (±)-1-((2-azaspiro[3.3]heptan-6-yl)oxy)-5,6,8,9,10,10a-hexahydropyrrolo[2,1-f][1,6]naphthyridine xTFA (10 g, quantitative yield) as a gummy thick liquid. This material was used without further purification. LCMS (Method H): Rt=0.42-0.61 min, m/z=300.1 [M+H]⁺, 91.76%.

### Step 2. (±)-1-((2-(3,6-Dichloro-1,2,4-triazin-5-yl)-2-azaspiro[3.3]heptan-6-yl)oxy)-5,6,8,9,10,10α-hexahydropyrrolo[2,1-f][1,6]naphthyridine

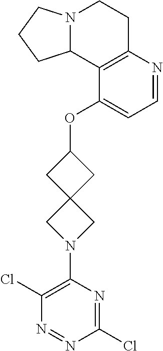 [SMILES: Clc1nnc(Cl)c(N2CC3(CC(Oc4ccnc5c4C4CCCN4CC5)C3)C2)n1]

To a stirred solution of 1-((2-azaspiro[3.3]heptan-6-yl)oxy)-5,6,8,9,10,10a-hexahydropyrrolo[2,1-f][1,6]naphthyridine xTFA (10 g, 15.94 mmol) in DCM (130 mL), TEA (66.6 mL, 478 mmol) was added. To this reaction mixture, 3,5,6-trichloro-1,2,4-triazine (2.351 g, 12.75 mmol) was added at 0° C. The reaction was stirred at the same temperature for 20 min under a nitrogen atmosphere. The reaction was monitored by TLC (10% MeOH in DCM) and LCMS. After completion, the reaction was quenched with water (10 mL) and the mixture was extracted with DCM (3×50 mL). The combined organic layer was dried over anhydrous sodium sulfate and filtered, and the filtrate was concentrated under reduced pressure. The crude was purified by reverse phase chromatography using a SiliCycle SiliaSep C18 column (120 g) on Biotage isolera one (Mobile phase A: 0.1% FA in water, B: ACN). The pure fractions were concentrated under reduced pressure and neutralized with saturated sodium bicarbonate solution. The mixture was extracted with 10% MeOH in DCM. The combined organic layer was dried over anhydrous sodium sulfate and filtered, and the filtrate was concentrated under reduced pressure to obtain (i)-1-((2-(3,6-dichloro-1,2,4-triazin-5-yl)-2-azaspiro[3.3]heptan-6-yl)oxy)-5,6,8,9,10,10a-hexahydropyrrolo[2,1-f][1,6]naphthyridine (3.5 g, 49.7% yield) as a solid. ¹H NMR (400 MHz, DMSO-d₆): δ 8.19 (d, J=5.6 Hz, 1H), 6.68 (d, J=5.6 Hz, 1H), 4.86-4.69 (m, 3H), 4.28-4.15 (m, 2H), 3.67 (br t, J=8.2 Hz, 1H), 3.02-2.75 (m, 6H), 2.73-2.62 (m, 2H), 2.47-2.39 (m, 1H), 2.37-2.27 (m, 2H), 1.89-1.68 (m, 2H), 1.56-1.42 (m, 1H); LCMS (Method B): Rt=0.94 min, m/z=433.2 [M+H]⁺, 98.00%.

### Intermediate 57. 5-Fluoro-2-hydroxy-N-isopropyl-N-(5-methyl-5-azaspiro[3.5]nonan-2-yl)benzamide

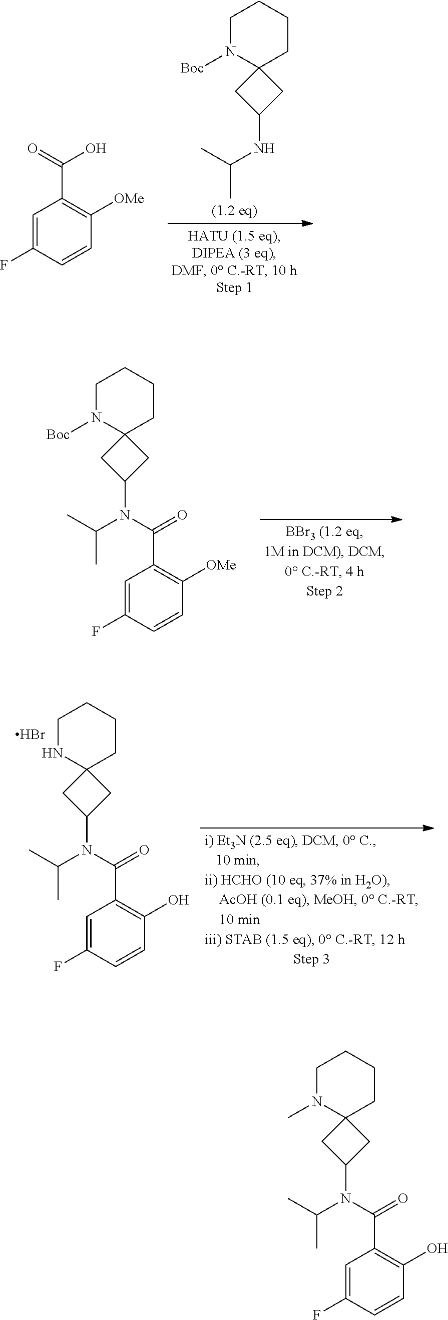 [SMILES: COc1ccc(F)cc1C(=O)O ; CC(C)NC1CC2(CCCCN2C(=O)OC(C)(C)C)C1 ; COc1ccc(F)cc1C(=O)N(C(C)C)C1CC2(CCCCN2C(=O)OC(C)(C)C)C1 ; Br ; CC(C)N(C(=O)c1cc(F)ccc1O)C1CC2(CCCCN2)C1 ; CC(C)N(C(=O)c1cc(F)ccc1O)C1CC2(CCCCN2C)C1]

### Step 1. tert-Butyl 2-(5-fluoro-N-isopropyl-2-methoxybenzamido)-5-azaspiro[3.5]nonane-5-carboxylate

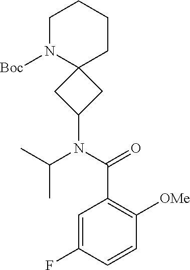 [SMILES: COc1ccc(F)cc1C(=O)N(C(C)C)C1CC2(CCCCN2C(=O)OC(C)(C)C)C1]

This compound was synthesized following the procedure described for the synthesis of Intermediate 43, Step 1, starting with 1 equivalent of 5-fluoro-2-methoxybenzoic acid and 1.2 equivalents of tert-butyl 2-(isopropylamino)-5-azaspiro[3.5]nonane-5-carboxylate. After work-up, the crude was purified by silica gel flash column chromatography using EtOAc in hexane (product eluted at 30% EtOAc in hexane) to obtain tert-butyl 2-(5-fluoro-N-isopropyl-2-methoxybenzamido)-5-azaspiro[3.5]nonane-5-carboxylate as a solid.

Yield: 68.5%; LCMS (Method A): Peak 1: Rt=2.32 min, 50.01%; Peak 2: Rt=2.38 min, 49.99%, m/z=435.3 [M+H]⁺.

### Step 2 5-Fluoro-2-hydroxy-N-isopropyl-N-(5-azaspiro[3.5]nonan-2-yl)benzamide Hydrobromide

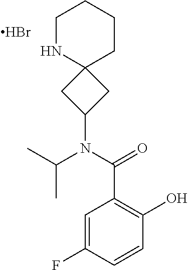 [SMILES: Br ; CC(C)N(C(=O)c1cc(F)ccc1O)C1CC2(CCCCN2)C1]

This compound was synthesized following the procedure described for the synthesis of Intermediate 43, Step 2, starting with 1 equivalent of tert-butyl 2-(5-fluoro-N-isopropyl-2-methoxybenzamido)-5-azaspiro[3.5]nonane-5-carboxylate, except that 1.2 equivalents of BBr₃ (1 M in DCM) was used. After completion, the reaction was quenched with MeOH and the mixture was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN) to obtain 5-fluoro-2-hydroxy-N-isopropyl-N-(5-azaspiro[3.5]nonan-2-yl)benzamide hydrobromide as a solid.

Yield: 62.9%; ¹H NMR (400 MHz, DMSO-d₆): δ 9.71 (br s, 1H), 8.58-7.39 (m, 2H), 7.09-7.00 (m, 1H), 6.99-6.82 (m, 2H), 4.37-3.85 (m, 1H), 3.80-3.55 (m, 1H), 3.11-2.79 (m, 3H), 2.37-2.21 (m, 2H), 1.92-1.35 (m, 6H), 1.28-0.93 (m, 6H), one proton merged with solvent peaks; LCMS (Method A): Peak 1 & Peak 2: Rt=1.47-1.58 min, m/z=321.1 [M+H]⁺, 81.27%.

### Step 3. 5-Fluoro-2-hydroxy-N-isopropyl-N-(5-methyl-5-azaspiro[3.5]nonan-2-yl)benzamide

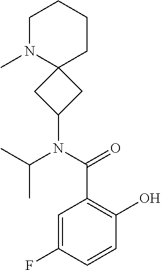 [SMILES: CC(C)N(C(=O)c1cc(F)ccc1O)C1CC2(CCCCN2C)C1]

To a stirred solution of 5-fluoro-2-hydroxy-N-isopropyl-N-(5-azaspiro[3.5]nonan-2-yl)benzamide hydrobromide (1.5 g, 4.20 mmol)) in DCM (15 mL), triethylamine (1.477 mL, 10.51 mmol) was added at 0° C. The reaction was stirred at RT for 10 min, then was concentrated under reduced pressure. The residue was dissolved in MeOH (15 mL), formaldehyde (3.31 mL, 42.0 mmol, 37% in H₂O) and acetic acid (0.024 mL, 0.420 mmol) were added at 0° C., and the reaction was stirred at the same temperature for 10 min. To this reaction mixture, sodium triacetoxyborohydride (1.336 g, 6.30 mmol) was added at 0° C., and the reaction was continued at RT for 12 h. The reaction progress was monitored by TLC (10% MeOH in DCM). After completion, the reaction was concentrated under reduced pressure. To this residue, water (50 mL) was added, and the mixture was extracted with 10% MeOH in DCM (3×75 mL). The combined organic layer was dried over sodium sulfate and filtered, and the filtrate was concentrated under reduced pressure. The crude (3.5 g) was purified by reverse phase chromatography using a Silicycle Siliasep C18 column (120 g) cartridge on Biotage isolera one (Mobile phase A: 10 mM NH₄HCO₃ in water, B: ACN) to obtain 5-fluoro-2-hydroxy-N-isopropyl-N-(5-methyl-5-azaspiro[3.5]nonan-2-yl)benzamide (1.1 g, 78% yield)) as a solid. ¹H NMR (400 MHz, DMSO-d₆): δ 9.66 (br s, 1H), 7.05-6.97 (m, 1H), 6.90-6.79 (m, 2H), 3.96-3.74 (m, 1H), 3.72-3.56 (m, 2H), 2.35 (br s, 2H), 2.31-2.14 (m, 4H), 1.81-1.59 (m, 2H), 1.52-1.03 (m, 10H), two protons merged with solvent peaks; LCMS (Method A): Rt=1.64 min, m/z=335.1 [M+H]⁺, 99.44%.

### Example 1. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-(2,2,2-trifluoroacetyl)azetidin-3-yl)benzamide (Compound No. 1)

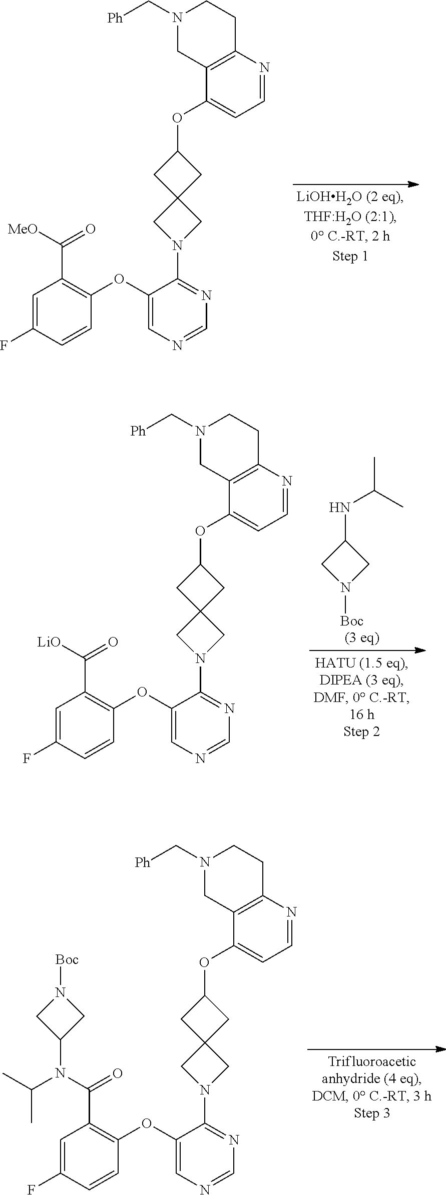

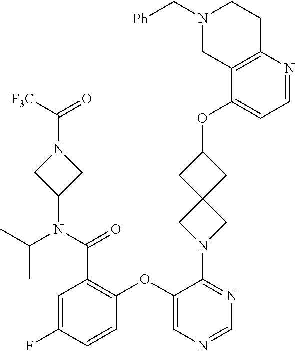 [SMILES: CC(C)N(C(=O)c1cc(F)ccc1Oc1cncnc1N1CC2(CC(Oc3ccnc4c3CN(Cc3ccccc3)CC4)C2)C1)C1CN(C(=O)C(F)(F)F)C1]

### Step 1. Lithium 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobutanoate

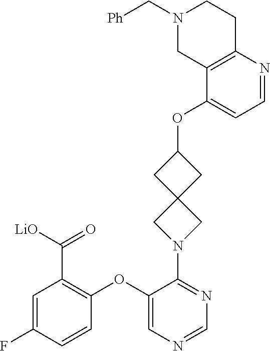

To a stirred solution of methyl 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoate (900 mg, 1.547 mmol) in THF:H₂O (2:1, 15 mL), LiOH·H₂O (130 mg, 3.09 mmol) was added at 0° C. The reaction was stirred at RT for 2 h. The reaction progress was monitored by TLC (10% MeOH in DCM). After completion, the reaction was concentrated under reduced pressure, and the residue was re-concentrated from toluene (3×20 mL) to obtain crude lithium 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoate (900 mg, 79% yield) as a solid. This material was used without further purification. LCMS (Method A): Rt=1.43 min, m/z=568.2 [M+H]⁺, 77.84%.

### Step 2. tert-Butyl 3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)azetidine-1-carboxylate

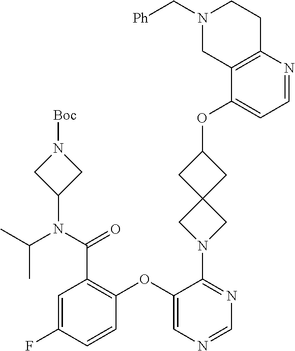 [SMILES: CC(C)N(C(=O)c1cc(F)ccc1Oc1cncnc1N1CC2(CC(Oc3ccnc4c3CN(Cc3ccccc3)CC4)C2)C1)C1CN(C(=O)OC(C)(C)C)C1]

In a dried, 25 mL two neck round bottom flask under a nitrogen atmosphere, 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid (350 mg, 0.617 mmol) and tert-butyl 3-(isopropylamino)azetidine-1-carboxylate (396 mg, 1.850 mmol) were dissolved in DMF (4 mL). To this reaction mixture, DIPEA (239 mg, 1.850 mmol) and HATU (352 mg, 0.925 mmol) were added at 0° C. and the reaction was stirred at RT for 16 h. The reaction progress was monitored by TLC (10% MeOH in DCM). After completion, the reaction was diluted with water (20 mL) and the mixture was extracted with EtOAc (3×20 mL). The combined organic layer was washed with cold water (50 mL) and brine solution (30 mL), dried over anhydrous sodium sulfate and filtered. The filtrate was concentrated under reduced pressure. The crude product was purified by silica gel flash column chromatography using MeOH in DCM (product eluted at 11% MeOH in DCM) to obtain tert-butyl 3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)azetidine-1-carboxylate (300 mg, 41.4% yield) as a gummy liquid. LCMS (Method A): Rt=2.28 min, m/z=764.3 [M+H]⁺, 65.17%.

### Step 3. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-(2,2,2-trifluoroacetyl)azetidin-3-yl)benzamide

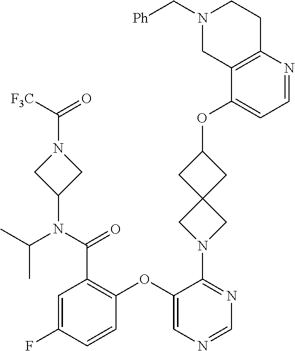 [SMILES: CC(C)N(C(=O)c1cc(F)ccc1Oc1cncnc1N1CC2(CC(Oc3ccnc4c3CN(Cc3ccccc3)CC4)C2)C1)C1CN(C(=O)C(F)(F)F)C1]

In a dried, 25 mL round bottom flask under a nitrogen atmosphere, tert-butyl 3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)azetidine-1-carboxylate (200 mg, 0.262 mmol) was dissolved in DCM (2 mL). To this solution, trifluoroacetic anhydride (0.148 mL, 1.047 mmol) was added at 0° C., and the reaction was stirred at RT for 3 h. The reaction progress was monitored by LCMS and TLC (10% MeOH in DCM). After completion, the reaction was concentrated and re-concentrated from EtOAc (2×10 mL) to obtain crude 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-(2,2,2-trifluoroacetyl)azetidin-3-yl)benzamide (145 mg). The crude (45 mg) was purified by reverse phase chromatography using a SiliCycle SiliaSep C18 column (40 g) on Biotage isolera one (Mobile phase A: 10 mM NH₄HCO₃ in water, B: ACN) to obtain 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-(2,2,2-trifluoroacetyl)azetidin-3-yl)benzamide (16.45 mg, 8.26% yield) as a solid. ¹H NMR (400 MHz, DMSO-d₆): δ 8.35-8.26 (m, 1H), 8.18 (d, J=5.5 Hz, 1H), 7.86-7.74 (m, 1H), 7.43 (dd, J=3.0, 8.3 Hz, 1H), 7.37-7.21 (m, 6H), 7.13-6.98 (m, 1H), 6.73-6.57 (m, 1H), 5.04-4.75 (m, 1H), 4.74-4.66 (m, 1H), 4.62-4.42 (m, 2H), 4.34-3.87 (m, 5H), 3.79-3.71 (m, 1H), 3.68 (s, 2H), 3.46 (br s, 2H), 2.92-2.64 (m, 7H), 2.27-2.10 (m, 2H), 1.56-0.89 (m, 6H); LCMS (Method A): Rt=2.14 min, m/z=760.3 [M+H]⁺; HPLC (Method A): Rt=6.50 min, 99.22%. The remaining crude product (100 mg) was used without further purification to prepare other examples.

### Example 2. N-(Azetidin-3-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 2)

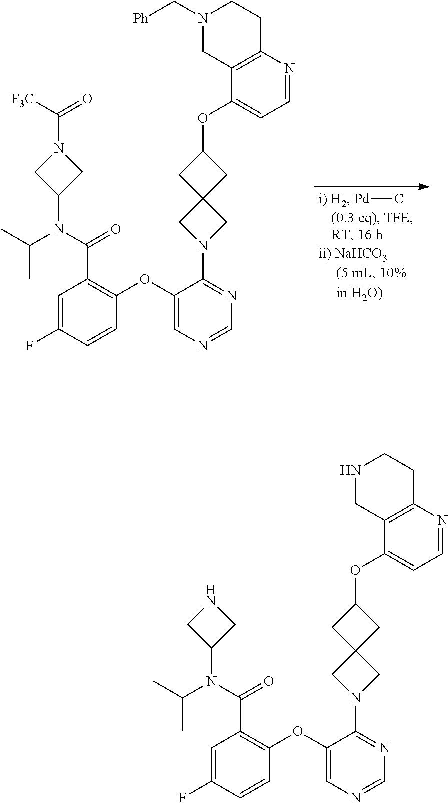

In a round bottom flask under a nitrogen atmosphere, 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-(2,2,2-trifluoroacetyl)azetidin-3-yl)benzamide (90 mg, 0.118 mmol) was dissolved in TFE (2 mL). To this solution, Pd—C (37.8 mg, 0.036 mmol) was added, and the reaction was stirred under a hydrogen atmosphere (balloon pressure) at RT for 16 h. The reaction progress was monitored by TLC (20% MeOH in DCM). After completion, the reaction was filtered through a Celite® pad, and the pad was washed with MeOH (30 mL). The filtrate was concentrated under reduced pressure, and the residue was diluted with 10% aqueous NaHCO₃ solution (5 mL). The mixture was extracted with 10% MeOH in DCM (3×10 mL). The combined organic layer was dried over anhydrous sodium sulfate and filtered, and the filtrate was concentrated under reduced pressure. The crude compound was purified by reverse phase chromatography using a SiliCycle SiliaSep C18 column (40 g) on Biotage isolera one (Mobile phase A: 10 mM NH₄HCO₃ in water, B: ACN), then by prep HPLC (Method B) to obtain N-(azetidin-3-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (5.79 mg, 8.35% yield) as a solid. ¹H NMR (400 MHz, DMSO-d₆): δ 8.33-8.24 (m, 1H), 8.16 (d, J=5.6 Hz, 1H), 7.82-7.68 (m, 1H), 7.38-7.15 (m, 2H), 7.13-6.99 (m, 1H), 6.64 (d, J=5.6 Hz, 1H), 4.73 (quin, J=6.5 Hz, 1H), 4.48-4.36 (m, 1H), 4.33-4.05 (m, 6H), 3.70 (br s, 3H), 3.58-3.49 (m, 1H), 3.00-2.92 (m, 2H), 2.83-2.73 (m, 2H), 2.71-2.64 (m, 3H), 2.28-2.20 (m, 2H), 1.56-0.92 (m, 6H), two protons merged with solvent peaks; LCMS (Method I): Rt=3.91 min, m/z=574.1 [M+H]⁺; HPLC (Method A): Rt=3.72 min, 97.64%.

### Example 3. N-(1-Acetylazetidin-3-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 3)

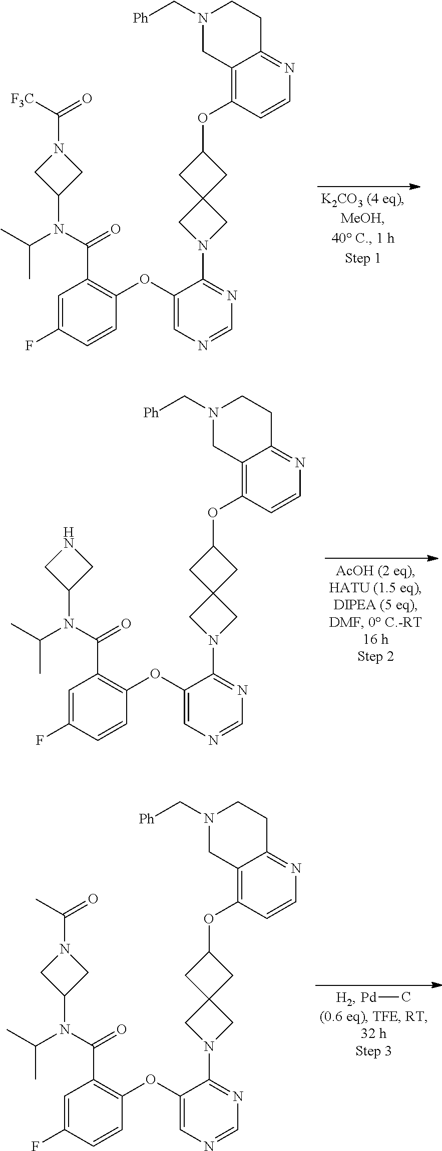

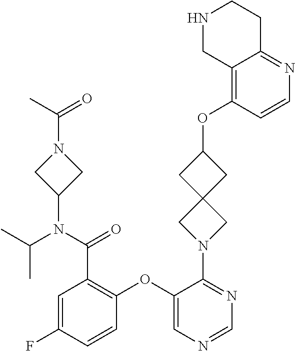 [SMILES: CC(=O)N1CC(N(C(=O)c2cc(F)ccc2Oc2cncnc2N2CC3(CC(Oc4ccnc5c4CNCC5)C3)C2)C(C)C)C1]

### Step 1. N-(Azetidin-3-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide

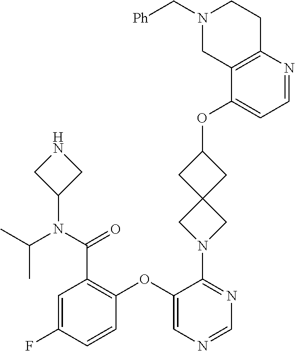 [SMILES: CC(C)N(C(=O)c1cc(F)ccc1Oc1cncnc1N1CC2(CC(Oc3ccnc4c3CN(Cc3ccccc3)CC4)C2)C1)C1CNC1]

In a dried, 25 mL round bottom flask under a nitrogen atmosphere, 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-(2,2,2-trifluoroacetyl)azetidin-3-yl)benzamide (600 mg, 0.790 mmol) was dissolved in MeOH (6 mL). To this solution, K₂CO₃ (437 mg, 3.16 mmol) was added at RT, and the reaction was stirred at 40° C. for 1 h. The reaction progress was monitored by TLC (10% MeOH in DCM). After completion, the reaction was diluted with water (5 mL), and the mixture was extracted with DCM (3×15 mL). The combined organic layer was dried over sodium sulfate and filtered, and the filtrate was concentrated under reduced pressure to obtain crude N-(azetidin-3-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (350 mg, 40% yield). This material was used without further purification. LCMS (Method G): Rt=1.80 min, m/z=664.4 [M+H]⁺, 59.92%.

### Step 2. N-(1-Acetylazetidin-3-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide

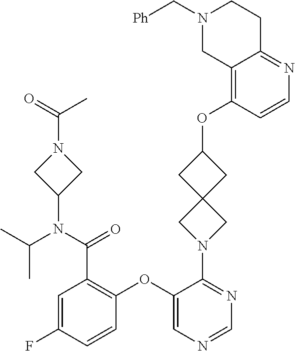 [SMILES: CC(=O)N1CC(N(C(=O)c2cc(F)ccc2Oc2cncnc2N2CC3(CC(Oc4ccnc5c4CN(Cc4ccccc4)CC5)C3)C2)C(C)C)C1]

To a stirred solution of N-(azetidin-3-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (150 mg, 0.226 mmol) and acetic acid (0.026 mL, 0.452 mmol) in DMF (3 mL), DIPEA (0.201 mL, 1.130 mmol) and HATU (129 mg, 0.339 mmol) were added at 0° C. The reaction was stirred at RT for 16 h. The reaction progress was monitored by TLC (10% MeOH in DCM). After completion, the reaction was concentrated under reduced pressure. The crude compound was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford N-(1-acetylazetidin-3-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (85 mg, 52.4% yield) as a solid. LCMS (Method F): Rt=2.05 min, m/z=706.5 [M+H]⁺, 98.30%.

### Step 3. N-(1-Acetylazetidin-3-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

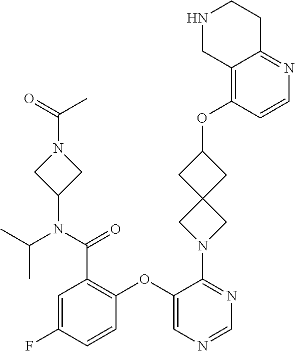 [SMILES: CC(=O)N1CC(N(C(=O)c2cc(F)ccc2Oc2cncnc2N2CC3(CC(Oc4ccnc5c4CNCC5)C3)C2)C(C)C)C1]

In a round bottom flask under a nitrogen atmosphere, N-(1-acetylazetidin-3-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (85 mg, 0.120 mmol) was dissolved in TFE (2 mL). To this solution, Pd—C (38.4 mg, 0.036 mmol) was added, and the reaction was stirred under a hydrogen atmosphere (balloon pressure) at RT for 16 h. The reaction progress was monitored by LCMS and TLC (20% MeOH in DCM). LCMS showed 49.64% product formation and 46.16% SM intact. Therefore, the reaction was filtered through a Celite® pad, and the pad was washed with MeOH (10 mL). The filtrate was concentrated under reduced pressure, and the crude was dissolved in TFE (2 mL). To this reaction mixture, fresh Pd—C (36.2 mg, 0.034 mmol) was added under a nitrogen atmosphere. The reaction was stirred under a hydrogen atmosphere (balloon pressure) at RT for another 16 h. The reaction progress was monitored by TLC (20% MeOH in DCM). After completion, the reaction was filtered through a Celite® pad, and the pad was washed with MeOH (10 mL). The filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain N-(1-acetylazetidin-3-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (28.79 mg, 40.9% yield) as a solid. ¹H NMR (400 MHz, DMSO-d₆): δ 8.34-8.26 (m, 1H), 8.15 (d, J=5.5 Hz, 1H), 7.93-7.71 (m, 1H), 7.39 (m, 1H), 7.28 (m, 1H), 7.05 (m, 1H), 6.71-6.53 (m, 1H), 4.78-4.68 (m, 2H), 4.60-4.43 (m, 1H), 4.37-3.90 (m, 6H), 4.01-3.89 (m, 1H), 3.86-3.72 (m, 3H), 2.94 (br t, J=5.7 Hz, 2H), 2.83-2.75 (m, 2H), 2.70-2.64 (m, 2H), 2.27-2.21 (m, 2H), 1.79-1.70 (m, 3H), 1.54-0.89 (m, 6H), one proton merged with solvent peaks; LCMS (Method F): Rt=1.56 min, m/z=616.4 [M+H]⁺; HPLC (Method A): Rt=3.97 min, 99.58%.

### Example 4. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(1-methylazetidin-3-yl)benzamide (Compound No. 4)

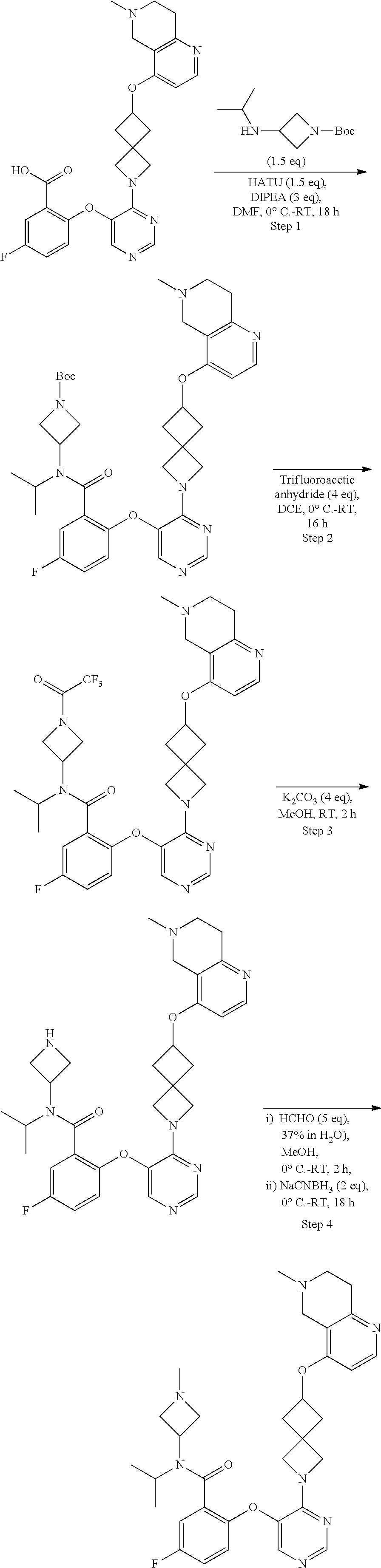

### Step 1. tert-Butyl 3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)azetidine-1-carboxylate

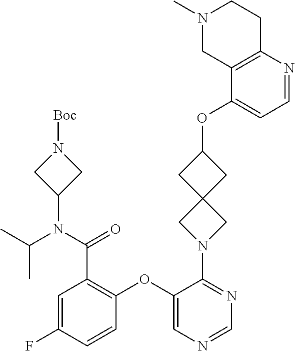 [SMILES: CC(C)N(C(=O)c1cc(F)ccc1Oc1cncnc1N1CC2(CC(Oc3ccnc4c3CN(C)CC4)C2)C1)C1CN(C(=O)OC(C)(C)C)C1]

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.5 equivalents of tert-butyl 3-(isopropylamino)azetidine-1-carboxylate. After work-up, the filtrate was concentrated under reduced pressure to afford crude tert-butyl 3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)azetidine-1-carboxylate (32.2% yield). This material was used without further purification. LCMS (Method A): Rt=1.70 min, m/z=688.3 [M+H]⁺, 90.22%.

### Step 2. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(1-(2,2,2-trifluoroacetyl)azetidin-3-yl)benzamide

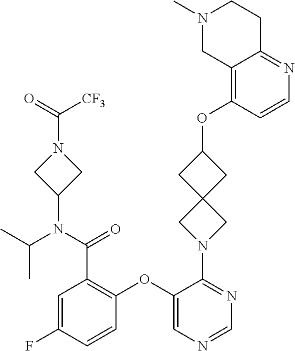 [SMILES: CC(C)N(C(=O)c1cc(F)ccc1Oc1cncnc1N1CC2(CC(Oc3ccnc4c3CN(C)CC4)C2)C1)C1CN(C(=O)C(F)(F)F)C1]

To a stirred solution of tert-butyl 3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)azetidine-1-carboxylate (0.45 g, 0.654 mmol) in DCE (14 mL), trifluoroacetic anhydride (0.369 mL, 2.62 mmol) was added at 0° C. The reaction was stirred at RT for 16 h, and the reaction progress was monitored by LCMS and TLC (10% MeOH in DCM). After completion, the reaction was concentrated under reduced pressure to afford crude 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(1-(2,2,2-trifluoroacetyl)azetidin-3-yl)benzamide (500 mg, 58.3% yield). This material was used without further purification. LCMS (Method B): Rt=1.19 min, m/z=684.1 [M+H]⁺, 52.19%.

### Step 3. N-(Azetidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

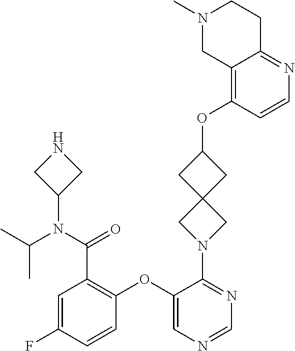 [SMILES: CC(C)N(C(=O)c1cc(F)ccc1Oc1cncnc1N1CC2(CC(Oc3ccnc4c3CN(C)CC4)C2)C1)C1CNC1]

To a stirred solution of 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(1-(2,2,2-trifluoroacetyl)azetidin-3-yl)benzamide (0.5 g, 0.731 mmol) in MeOH (20 mL), K₂CO₃ (0.404 g, 2.93 mmol) was added at 0° C. The reaction was stirred at RT for 4 h, and the reaction progress was monitored by LCMS and TLC (10% MeOH in DCM). After completion, the reaction was concentrated under reduced pressure, and water (10 mL) was added to the residue. The mixture was extracted with 5% MeOH in DCM (2×30 mL). The combined organic layer was concentrated under reduced pressure to afford crude N-(azetidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (285 mg, 46.1% yield). This material was used without further purification. ¹H NMR (400 MHz, DMSO-d₆): δ 8.32-8.25 (m, 1H), 8.18 (d, J=5.5 Hz, 1H), 7.79-7.63 (m, 1H), 7.36-6.96 (m, 3H), 6.72-6.64 (m, 1H), 4.82-4.68 (m, 1H), 4.32-4.07 (m, 5H), 3.74-3.42 (m, 2H), 3.37 (s, 2H), 2.87-2.73 (m, 4H), 2.63 (br t, J=5.8 Hz, 2H), 2.40-2.34 (m, 3H), 2.30-2.21 (m, 2H), 1.53-0.83 (m, 6H), four protons merged with solvent peaks; LCMS (Method B): Rt=0.29-1.05 min, m/z=588.2 [M+H]⁺, 69.61%.

### Step 4. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(1-methylazetidin-3-yl)benzamide

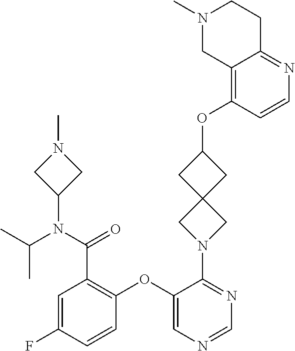 [SMILES: CC(C)N(C(=O)c1cc(F)ccc1Oc1cncnc1N1CC2(CC(Oc3ccnc4c3CN(C)CC4)C2)C1)C1CN(C)C1]

To a stirred solution of N-(azetidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (260 mg, 0.442 mmol) in MeOH (5 mL), formaldehyde (0.165 mL, 2.212 mmol, 37% in H₂O) was added at 0° C. under a nitrogen atmosphere. The reaction was stirred at RT for 2 h. Sodium cyanoborohydride (55.6 mg, 0.885 mmol) was added portion-wise to this reaction mixture at 0° C. The reaction was stirred at RT for 18 h. The reaction progress was monitored by TLC (10% MeOH in DCM). After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN), then by Prep HPLC (Method C). ¹H NMR of this product showed an impurity. Therefore, the product was re-purified by reverse phase chromatography using a SiliCycle SiliaSep C18 column (12 g) on Biotage isolera one (Mobile phase A: 10 mM NH₄HCO₃ in water, B: ACN). The pure fractions were concentrated under reduced pressure and lyophilized to obtain 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(1-methylazetidin-3-yl)benzamide (9 mg, 3.36% yield) as a solid. ¹H NMR (400 MHz, DMSO-d₆): δ 8.28 (br d, J=9.6 Hz, 1H), 8.18 (d, J=5.6 Hz, 1H), 7.77-7.71 (m, 1H), 7.36-7.16 (m, 2H), 7.12-7.03 (m, 1H), 6.66 (d, J=5.6 Hz, 1H), 4.75 (quin, J=6.6 Hz, 1H), 4.38-3.94 (m, 5H), 3.76-3.45 (m, 2H), 3.36 (s, 2H), 3.27-3.06 (m, 2H), 3.01-2.91 (m, 1H), 2.85-2.74 (m, 4H), 2.63 (br t, J=5.8 Hz, 2H), 2.36 (s, 3H), 2.31-2.13 (m, 5H), 1.51-0.91 (m, 6H); LCMS (Method C): Rt=1.47 min, m/z=602.4 [M+H]⁺, 99.39%; HPLC (Method A): Rt=4.51 min, 89.66%.

### Example 5. (R)-5-Fluoro-N-isopropyl-N-(pyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 5)

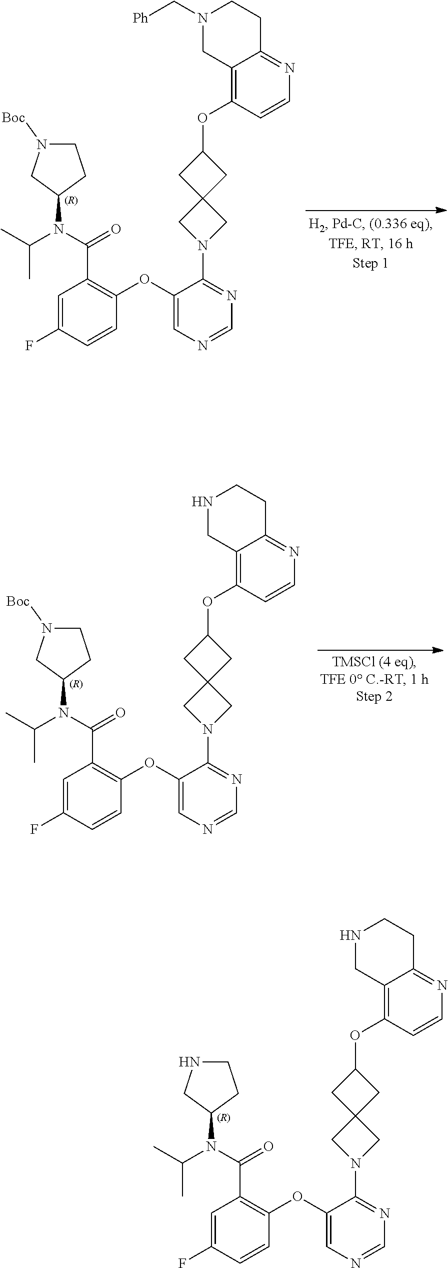

### Step 1. tert-Butyl (R)-3-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)pyrrolidine-1-carboxylate

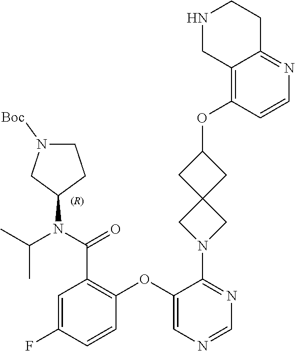

In a dried, round bottom flask under a nitrogen atmosphere, tert-butyl (R)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)pyrrolidine-1-carboxylate (0.125 g, 0.161 mmol) was dissolved in TFE (2 mL). To this solution, Pd—C (0.057 g, 0.054 mmol) was added, and the reaction was stirred under a hydrogen atmosphere (balloon pressure) at RT for 16 h. The reaction progress was monitored by LCMS and TLC (20% MeOH in DCM). After completion, the reaction was degassed with nitrogen for 3 min, then was filtered through a Celite® pad, and the pad was washed with MeOH (2×25 mL). The filtrate was concentrated under reduced pressure to afford crude tert-butyl (R)-3-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)pyrrolidine-1-carboxylate (90 mg, 72.0% yield). This material was used without further purification. LCMS (Method A): Rt=1.75 min, m/z=688.4 [M+H]⁺, 88.43%.

### Step 2. (R)—5-Fluoro-N-isopropyl-N-(pyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

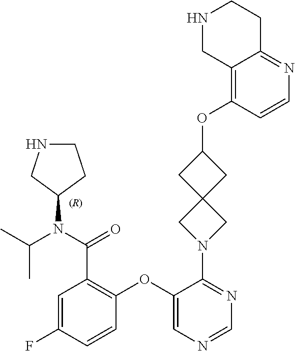

To a stirred solution of tert-butyl (R)-3-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)pyrrolidine-1-carboxylate (0.09 g, 0.131 mmol) in TFE (25 mL), TMSCl (0.057 g, 0.523 mmol) was added at 0° C. The reaction was stirred at RT for 1 h, with monitoring by LCMS. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain (R)-5-fluoro-N-isopropyl-N-(pyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (32.2 mg, 41.7% yield) as a solid. ¹H NMR (400 MHz, DMSO-d₆): δ 8.31-8.25 (m, 1H), 8.16 (d, J=5.5 Hz, 1H), 7.77-7.73 (m, 1H), 7.34-7.19 (m, 2H), 7.12-7.02 (m, 1H), 6.64 (br d, J=3.9 Hz, 1H), 4.73 (quin, J=6.3 Hz, 1H), 4.34-4.17 (m, 3H), 4.16-4.08 (m, 1H), 3.99-3.72 (m, 1H), 3.70 (br s, 2H), 3.19-3.05 (m, 2H), 3.00-2.89 (br d, J=5.1 Hz, 3H), 2.77 (br s, 3H), 2.71-2.62 (m, 3H), 2.29-2.18 (m, 2H), 2.13-1.54 (m, 2H), 1.47-0.97 (m, 6H), two protons merged with solvent peaks; LCMS (Method H): Rt=1.39 min, m/z=588.4 [M+H]⁺; HPLC (Method A): Rt=3.84 min, 99.51%.

### Example 6. (S)-5-Fluoro-N-isopropyl-N-(pyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 6)

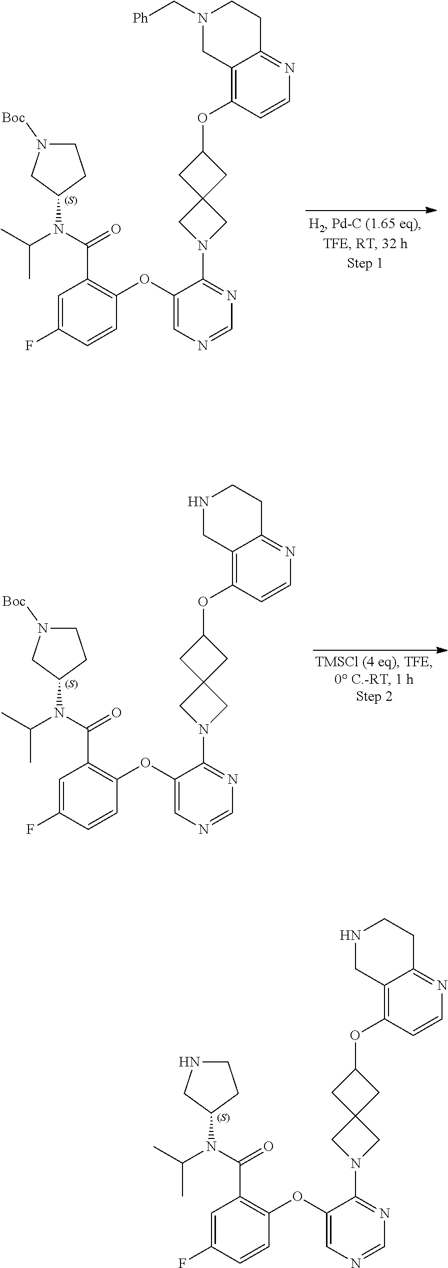

### Step 1. tert-Butyl (S)-3-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)pyrrolidine-1-carboxylate

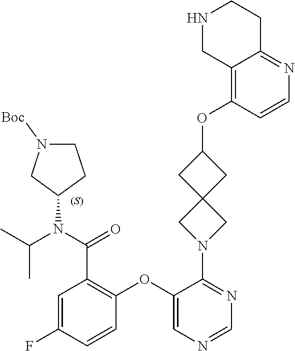

This compound was synthesized following the procedure described for the synthesis of Example 5, step 1, starting with 1 equivalent of (S)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)pyrrolidine-1-carboxylate, except that 0.351 equivalent of Pd—C was used. LCMS of the reaction showed 36% desired product and 56% SM intact. After filtration, the crude was dissolved in TFE, fresh Pd—C (1.3 equivalents) was added, and the reaction was stirred under a hydrogen atmosphere (balloon pressure). After filtration and concentration, the crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure to afford tert-butyl (S)-3-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)pyrrolidine-1-carboxylate (81% yield). LCMS (Method A): Rt=1.76 min, m/z=688.4 [M+H]⁺, 83.18%.

### Step 2. (S)—5-Fluoro-N-isopropyl-N-(pyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

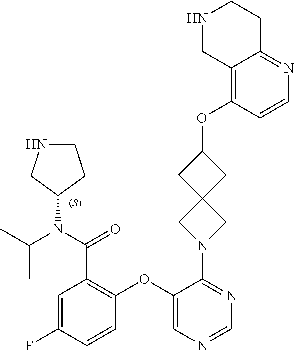

This compound was synthesized following the procedure described for the synthesis of Example 5, step 2, starting with 1 equivalent of tert-butyl (S)-3-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)pyrrolidine-1-carboxylate. After completion, the reaction was concentrated under reduced pressure and re-concentrated from EtOAc. The crude was purified by reverse phase chromatography using a SiliCycle SiliaSep C18 column (40 g) on Biotage isolera one (Mobile phase A: 10 mM NH₄HCO₃ in water, B: ACN). The fractions were concentrated under reduced and lyophilized to obtain (S)-5-fluoro-N-isopropyl-N-(pyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (23.34% yield). ¹H NMR (400 MHz, DMSO-d₆): δ 8.32-8.25 (m, 1H), 8.17 (br d, J=5.3 Hz, 1H), 7.82-7.71 (m, 1H), 7.36-7.18 (m, 2H), 7.12-7.00 (m, 1H), 6.66-6.61 (m, 1H), 4.79-4.68 (m, 1H), 4.31-4.07 (m, 4H), 3.98-3.83 (m, 1H), 3.77-3.66 (m, 4H), 3.07-2.92 (m, 3H), 2.82-2.74 (m, 3H), 2.70-2.64 (m, 3H), 2.27-2.20 (m, 2H), 2.02-1.85 (m, 2H), 1.50-0.97 (m, 6H), two protons merged with solvent peaks; LCMS (Method H): Rt=2.95 min, m/z=588.3 [M+H]⁺; HPLC (Method A): Rt=3.82 min, 98.79%; Chiral SFC (Method M): Rt=2.54 min, 100%.

### Example 7. N-((1r,3r)-3-Acetamidocyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 7)

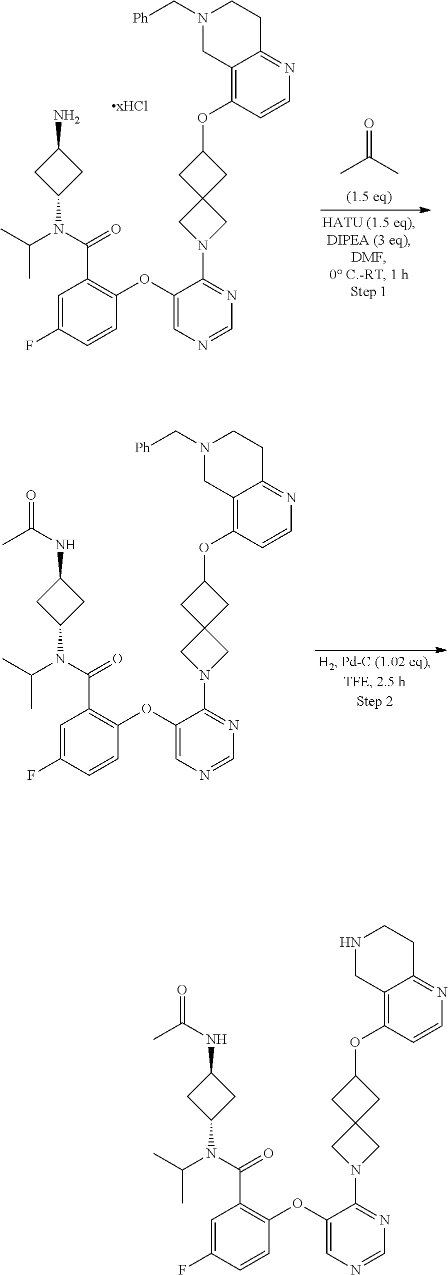

### Step 1. N-((1r,3r)-3-Acetamidocyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide

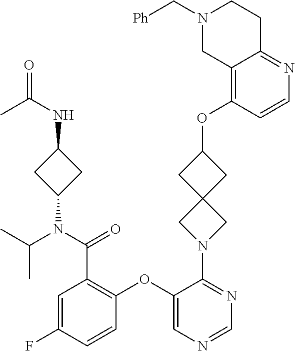 [SMILES: CC(=O)N[C@H]1C[C@H](N(C(=O)c2cc(F)ccc2Oc2cncnc2N2CC3(CC(Oc4ccnc5c4CN(Cc4ccccc4)CC5)C3)C2)C(C)C)C1]

The reaction was performed following the same procedure in three batches.

This compound was synthesized following the procedure described for the synthesis of Example 3, step 2, starting with 1 equivalent of N-((1r,3r)-3-aminocyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl and 1.5 equivalents of AcOH, except that 3 equivalents of DIPEA was used. After completion, the reaction was quenched with ice-cold water and the mixture was extracted with EtOAc. The combined organic layer was washed with brine solution, dried over anhydrous sodium sulfate and filtered, and the filtrate was concentrated under reduced pressure. After workup, the crude residue from three batches were combined and purified by reverse phase chromatography using a SiliCycle SiliaSep C18 column (80 g) on Biotage isolera one (Mobile phase A: 10 mM NH₄HCO₃ in water, B: ACN) to obtain N-((1r,3r)-3-acetamidocyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide as a solid.

Combined Yield: 53.07%; ¹H NMR (400 MHz, DMSO-d₆): δ 8.32-8.25 (m, 1H), 8.23-8.11 (m, 2H), 7.79-7.71 (m, 1H), 7.40-7.21 (m, 7H), 7.19-7.03 (m, 1H), 6.66 (d, J=5.6 Hz, 1H), 4.74-4.66 (m, 1H), 4.42-4.04 (m, 6H), 4.01-3.91 (m, 1H), 3.82-3.71 (m, 1H), 3.68 (s, 2H), 3.47 (s, 2H), 3.27-3.01 (m, 1H), 2.86-2.66 (m, 6H), 2.26-2.17 (m, 2H), 2.06-1.92 (m, 1H), 1.85-1.72 (m, 3H), 1.51-0.91 (m, 6H), one proton merged with solvent peaks; LCMS (Method A): Rt=1.82 min, m/z=720.4 [M+H]⁺, 99.46%.

### Step 2. N-((1r,3r)-3-Acetamidocyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

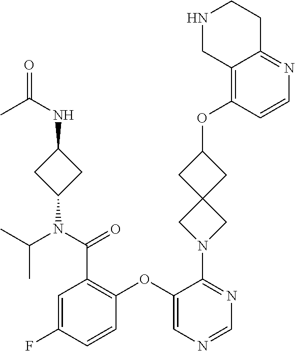 [SMILES: CC(=O)N[C@H]1C[C@H](N(C(=O)c2cc(F)ccc2Oc2cncnc2N2CC3(CC(Oc4ccnc5c4CNCC5)C3)C2)C(C)C)C1]

This compound was synthesized following the procedure described for the synthesis of Example 3, step 3, starting with 1 equivalent of N-((1r,3r)-3-acetamidocyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide, except that 1.02 equivalents of Pd—C was used. After completion, the reaction was filtered through a Celite® pad, and the pad was washed with TFE. The filtrate was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain N-((1r,3r)-3-acetamidocyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 69.7%; ¹H NMR (400 MHz, DMSO-d₆): δ 8.30-8.25 (m, 1H), 8.22-8.09 (m, 2H), 7.76 (s, 1H), 7.34-7.02 (m, 3H), 6.64 (d, J=5.6 Hz, 1H), 4.72 (quin, J=6.5 Hz, 1H), 4.40-4.06 (m, 6H), 4.02-3.71 (m, 1H), 3.70 (s, 2H), 3.26-2.99 (m, 1H), 2.95 (t, J=5.8 Hz, 2H), 2.83-2.72 (m, 2H), 2.68 (t, J=5.5 Hz, 2H), 2.47-2.31 (m, 1H), 2.29-2.16 (m, 2H), 2.12-1.84 (m, 2H), 1.83-1.72 (m, 3H), 1.49-1.26 (m, 3H), 1.07-0.95 (m, 3H), one proton merged with solvent peaks; LCMS (Method A): Rt=1.42 min, m/z=630.3 [M+H]⁺, 99.81%; HPLC (Method A): Rt=4.01 min, 99.85%; Chiral SFC (Method N): Rt=1.46 min, 100%.

### Example 8. N-((1s,3s)-3-Aminocyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 8)

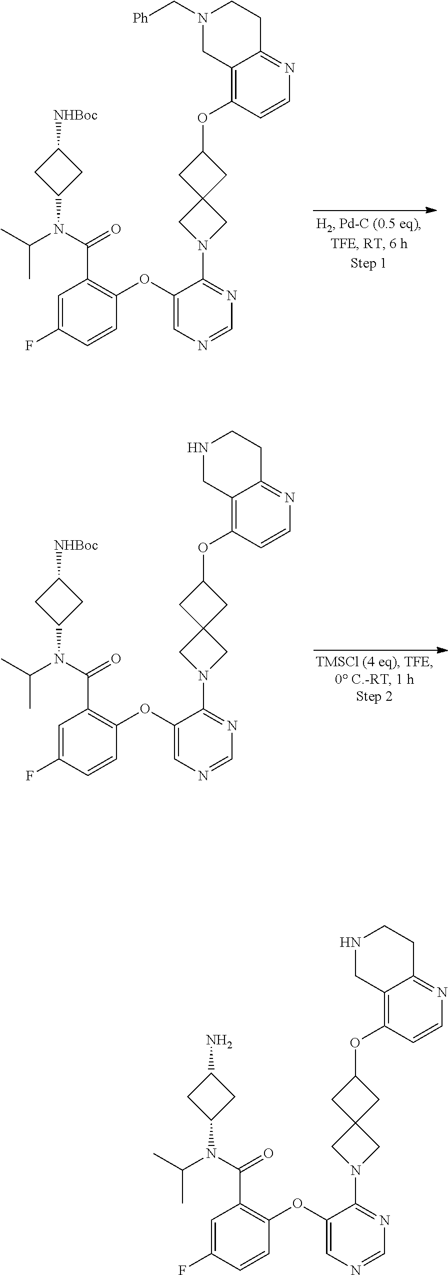

### Step 1. tert-Butyl ((1s,3s)-3-(5-fluoro-N-isopropyl-2-(4-(6-((5,6,7,8-tetrahydro-1,6-naphthyridin-4-yl)oxy)-2-azaspiro[3.3]heptan-2-yl)pyrimidin-5-yl)oxy)benzamido)cyclobutyl)carbamate

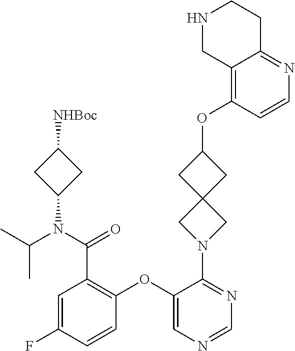 [SMILES: CC(C)N(C(=O)c1cc(F)ccc1Oc1cncnc1N1CC2(CC(Oc3ccnc4c3CNCC4)C2)C1)[C@H]1C[C@@H](NC(=O)OC(C)(C)C)C1]

This compound was synthesized following the procedure described for the synthesis of Example 5, step 1, starting with 1 equivalent of tert-butyl ((1s,3s)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclobutyl)carbamate, except that 0.5 equivalent of Pd—C was used. After filtration, the filtrate was concentrated under reduced pressure to obtain crude tert-butyl ((1s,3s)-3-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)cyclobutyl)carbamate as a solid. This material was used without further purification.

Yield: 92%; LCMS (Method A): Rt=1.73 min, m/z=688.2 [M+H]⁺, 93.91%.

### Step 2. N-((1s,3s)-3-Aminocyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

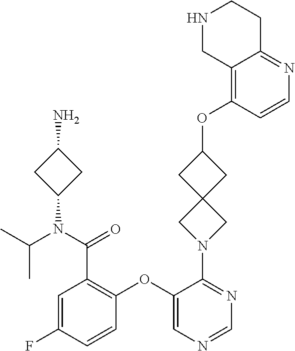 [SMILES: CC(C)N(C(=O)c1cc(F)ccc1Oc1cncnc1N1CC2(CC(Oc3ccnc4c3CNCC4)C2)C1)[C@H]1C[C@@H](N)C1]

This compound was synthesized following the procedure described for the synthesis of Example 5, step 2, starting with 1 equivalent of tert-butyl ((1s,3s)-3-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)cyclobutyl)carbamate. After completion, the reaction was concentrated under reduced pressure to obtain crude N-((1s,3s)-3-aminocyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl. The crude was purified by Prep HPLC (Method I) to afford N-((1s,3s)-3-aminocyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 37.3%; ¹H NMR (400 MHz, DMSO-d₆): δ 8.29-8.24 (m, 1H), 8.16 (d, J=5.6 Hz, 1H), 7.76-7.71 (m, 1H), 7.29-7.21 (m, 1H), 7.14-7.03 (m, 2H), 6.64 (d, J=5.6 Hz, 1H), 4.73 (quin, J=6.6 Hz, 1H), 4.34-4.08 (m, 4H), 3.95-3.83 (m, 1H), 3.70 (s, 2H), 3.61-3.49 (m, 1H), 2.95 (t, J=5.4 Hz, 2H), 2.83-2.72 (m, 3H), 2.68 (t, J=5.4 Hz, 2H), 2.43-2.35 (m, 1H), 2.29-2.17 (m, 3H), 2.11-2.01 (m, 1H), 1.82-1.67 (m, 2H), 1.47-0.95 (m, 6H), two protons merged with solvent peaks; LCMS (Method F): Rt=2.92 min, m/z=588.4 [M+H]⁺; HPLC (Method A): Rt=3.87 min, 99.48%; Chiral SFC (Method S): Rt=3.53 min, 98.95%.

### Example 9. N-((1r,3r)-3-Aminocyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 9)

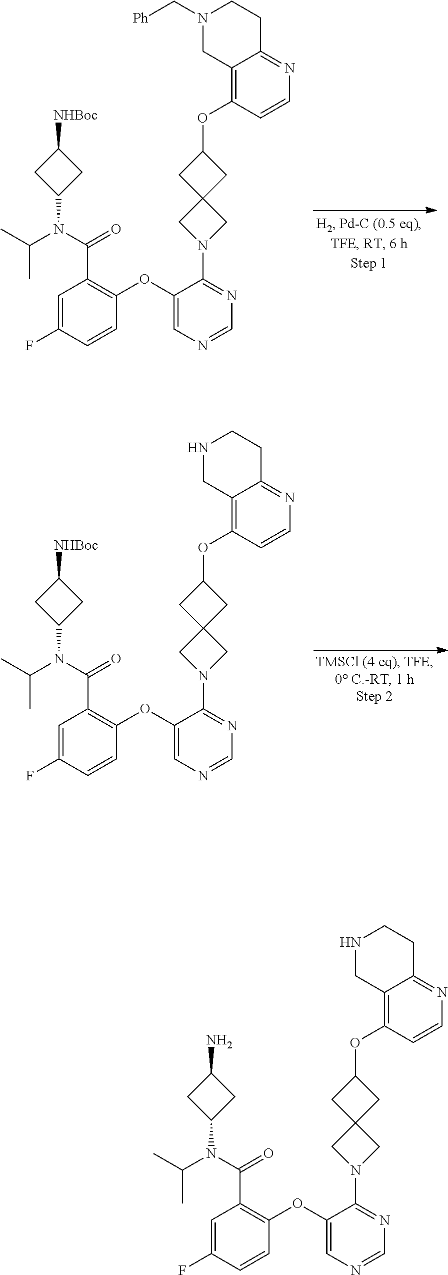

### Step 1. tert-Butyl ((1r,3r)-3-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)cyclobutyl)carbamate

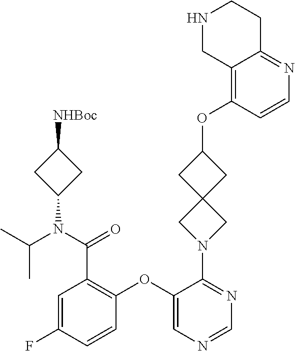 [SMILES: CC(C)N(C(=O)c1cc(F)ccc1Oc1cncnc1N1CC2(CC(Oc3ccnc4c3CNCC4)C2)C1)[C@H]1C[C@H](NC(=O)OC(C)(C)C)C1]

This compound was synthesized following the procedure described for the synthesis of Example 5, step 1, starting with 1 equivalent of tert-butyl ((1r,3r)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclobutyl)carbamate, except that 0.5 equivalent of Pd—C was used. After filtration and concentration, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl ((1r,3r)-3-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)cyclobutyl)carbamate as a solid.

Yield: 70.1%; LCMS (Method A): Rt=1.71 min, m/z=688.2 [M+H]⁺, 92.96%.

### Step 2. N-((1r,3r)-3-Aminocyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

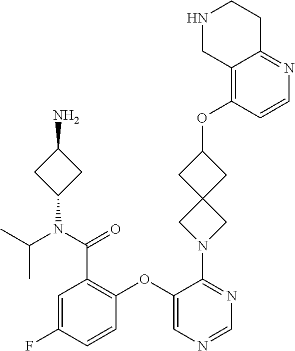 [SMILES: CC(C)N(C(=O)c1cc(F)ccc1Oc1cncnc1N1CC2(CC(Oc3ccnc4c3CNCC4)C2)C1)[C@H]1C[C@H](N)C1]

This compound was synthesized following the procedure described for the synthesis of Example 5, step 2, starting with 1 equivalent of tert-butyl ((1r,3r)-3-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)cyclobutyl)carbamate. After completion, the reaction was concentrated under reduced pressure to obtain crude N-((1r,3r)-3-aminocyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl. The crude was purified by Prep HPLC (Method B) to afford N-((1r,3r)-3-aminocyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 37.8%; ¹H NMR (400 MHz, DMSO-d₆): δ 8.28-8.26 (m, 1H), 8.16 (d, J=5.6 Hz, 1H), 7.74 (s, 1H), 7.30-7.21 (m, 1H), 7.14-7.02 (m, 2H), 6.64 (d, J=5.8 Hz, 1H), 4.73 (quin, J=6.5 Hz, 1H), 4.43-4.33 (m, 1H), 4.32-4.08 (m, 5H), 3.76-3.62 (m, 3H), 3.60-3.49 (m, 1H), 3.07-3.00 (m, 1H), 2.95 (t, J=5.6 Hz, 2H), 2.82-2.74 (m, 2H), 2.70-2.65 (m, 2H), 2.31-2.19 (m, 3H), 1.88-1.58 (m, 2H), 1.47-0.94 (m, 6H), two protons merged with solvent peaks; LCMS (Method F): Rt=1.54 min, m/z=588.4 [M+H]⁺; HPLC (Method A): Rt=3.79 min, 98.48%; Chiral SFC (Method A1): Rt=2.57 min, 97.07%.

### Example 10. N-((1s,3s)-3-(Dimethylamino)cyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 10)

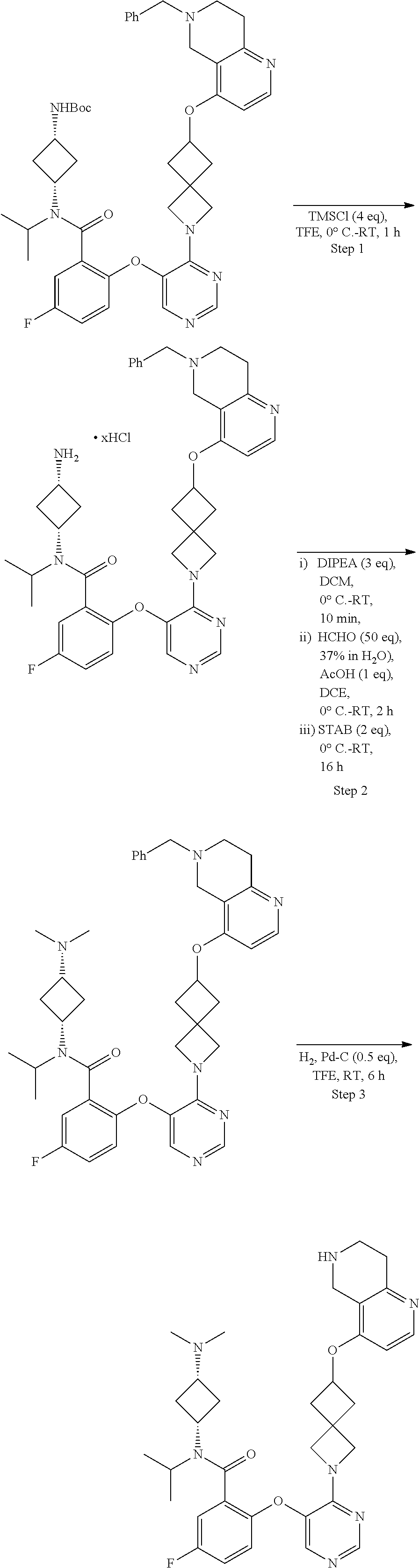

### Step 1. N-((1s,3s)-3-Aminocyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl

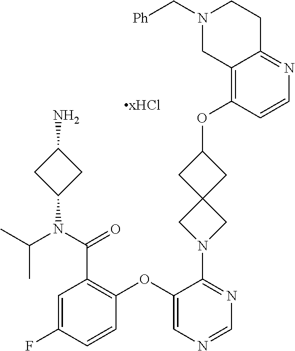 [SMILES: CC(C)N(C(=O)c1cc(F)ccc1Oc1cncnc1N1CC2(CC(Oc3ccnc4c3CN(Cc3ccccc3)CC4)C2)C1)[C@H]1C[C@@H](N)C1]

This compound was synthesized following the procedure described for the synthesis of Example 5, step 2, starting with 1 equivalent of tert-butyl ((1s,3s)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclobutyl)carbamate. After completion, the reaction was concentrated under reduced pressure to obtain crude N-((1s,3s)-3-aminocyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl as a solid. This material was used without further purification.

Yield: 87%; LCMS (Method G): Rt=1.94 min, m/z=678.8 [M+H]⁺, 89.08%.

### Step 2. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-N-((1s,3s)-3-(dimethylamino)cyclobutyl)-5-fluoro-N-isopropylbenzamide

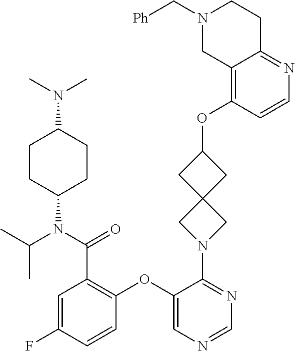

In a dried, round bottom flask under a nitrogen atmosphere, N-((1s,3s)-3-aminocyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl (0.360 g, 0.504 mmol) was dissolved in DCM (3 mL). To this reaction mixture, DIPEA (0.264 mL, 1.512 mmol) was added, and the reaction was stirred at RT for 10 min. The reaction was concentrated under reduced pressure, and the residue was dissolved in DCE (5 mL). To this reaction mixture, formaldehyde (1.876 mL, 25.2 mmol, 37% in H₂O) and acetic acid (0.029 mL, 0.504 mmol) were added at 0° C., and the reaction was stirred at RT for 2 h. The reaction was cooled to 0° C., STAB (0.214 g, 1.008 mmol) was added, and the reaction was stirred at RT for 16 h. The reaction progress was monitored by LCMS. After completion, the reaction was concentrated under reduced pressure. The crude product was purified by reverse phase chromatography using a SiliCycle SiliaSep C18 column (80 g) on Biotage isolera one (Mobile phase A: 10 mM NH₄HCO₃ in water, B: ACN) to obtain 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1s,3s)-3-(dimethylamino)cyclobutyl)-5-fluoro-N-isopropylbenzamide (150 mg, 38.4% yield) as a solid. ¹H NMR (400 MHz, DMSO-d₆): δ 8.30-8.25 (m, 1H), 8.19 (d, J=5.6 Hz, 1H), 7.77-7.74 (m, 1H), 7.35 (d, J=4.5 Hz, 5H), 7.30-7.23 (m, 2H), 7.16-7.06 (m, 1H), 6.66 (d, J=5.6 Hz, 1H), 4.72 (quin, J=6.6 Hz, 1H), 4.32-4.06 (m, 4H), 3.79-3.70 (m, 2H), 3.68 (s, 2H), 3.47 (s, 2H), 2.83-2.73 (m, 5H), 2.72-2.66 (m, 2H), 2.25-2.17 (m, 2H), 2.16-2.07 (m, 2H), 2.02 (s, 3H), 1.95 (s, 3H), 1.89-1.75 (m, 1H), 1.48-0.94 (m, 6H), one proton merged with solvent peaks; LCMS (Method F): Rt=2.07 min, m/z=706.4 [M+H]⁺, 91.12%.

### Step 3. N-((1s,3s)-3-(Dimethylamino)cyclobutyl)-5-fluoro-N-isopropyl-2-((4-(6-((5,6,7,8-tetrahydro-1,6-naphthyridine-4-yl)oxy)-2-azaspiro[3.3]heptan-2-yl)pyrimidin-5-yl)oxy)benzamide

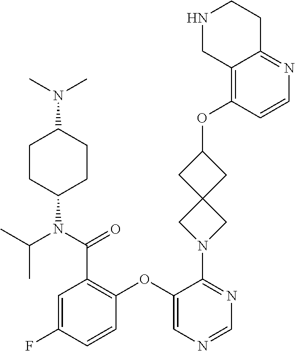

This compound was synthesized following the procedure described for the synthesis of Example 5, step 1, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1s,3s)-3-(dimethylamino)cyclobutyl)-5-fluoro-N-isopropylbenzamide, except that 0.5 equivalent of Pd—C was used. After filtration and concentration, the crude compound was purified by Prep HPLC (Method B) to afford N-((1s,3s)-3-(dimethylamino)cyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 25.5%; ¹H NMR (400 MHz, DMSO-d₆): δ 8.30-8.25 (m, 1H), 8.16 (d, 0.1=5.6 Hz, 1H), 7.78-7.74 (m, 1H), 7.31-7.21 (m, 1H), 7.17-7.02 (m, 2H), 6.64 (d, J=5.6 Hz, 1H), 4.77-4.69 (m, 1H), 4.36-4.07 (m, 4H), 3.79-3.52 (m, 4H), 2.96 (t, J=5.6 Hz, 2H), 2.83-2.74 (m, 3H), 2.71-2.65 (m, 2H), 2.43-2.35 (m, 1H), 2.29-2.08 (m, 5H), 2.04 (s, 3H), 1.96 (s, 3H), 1.88-1.76 (m, 1H), 1.46-0.97 (m, 6H); LCMS (Method K): Rt=4.92 min, m/z=616.1 [M+H]⁺; HPLC (Method A): Rt=4.28 min, 99.50%; Chiral SFC (Method AK): Rt=4.27 min, 100%.

### Example 11. N-((1r,3r)-3-(Dimethylamino)cyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 11)

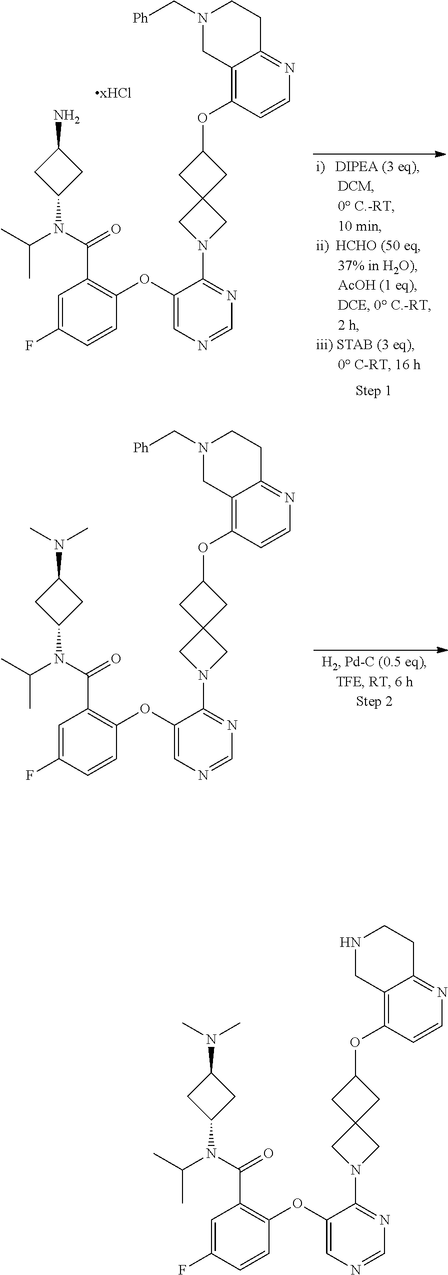 [SMILES: CC(C)N(C(=O)c1cc(F)ccc1Oc1cncnc1N1CC2(CC(Oc3ccnc4c3CN(Cc3ccccc3)CC4)C2)C1)[C@H]1C[C@H](N)C1 ; CC(C)N(C(=O)c1cc(F)ccc1Oc1cncnc1N1CC2(CC(Oc3ccnc4c3CN(Cc3ccccc3)CC4)C2)C1)[C@H]1C[C@H](N(C)C)C1 ; CC(C)N(C(=O)c1cc(F)ccc1Oc1cncnc1N1CC2(CC(Oc3ccnc4c3CNCC4)C2)C1)[C@H]1C[C@H](N(C)C)C1]

### Step 1. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(dimethylamino)cyclobutyl)-5-fluoro-N-isopropylbenzamide

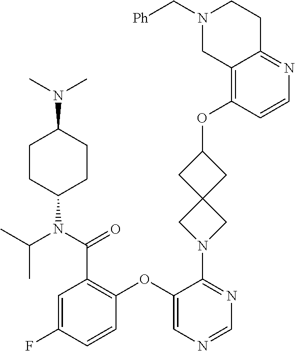 [SMILES: CC(C)N(C(=O)c1cc(F)ccc1Oc1cncnc1N1CC2(CC(Oc3ccnc4c3CN(Cc3ccccc3)CC4)C2)C1)[C@H]1CC[C@H](N(C)C)CC1]

This compound was synthesized following the procedure described for the synthesis of Example 10, step 2, starting with 1 equivalent of N-((1r,3r)-3-aminocyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl, except that 3 equivalents of STAB was used. After completion, the crude was purified by reverse phase chromatography using a SiliCycle SiliaSep C18 column (80 g) on Biotage isolera one (Mobile phase A: 10 mM NH₄HCO₃ in water, B: ACN) to obtain 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(dimethylamino)cyclobutyl)-5-fluoro-N-isopropylbenzamide as a solid.

Yield: 37.6%; LCMS (Method F): Rt=1.93 min, m/z=706.3 [M+H]⁺, 74.26%.

### Step 2. N-((1r,3r)-3-(Dimethylamino)cyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

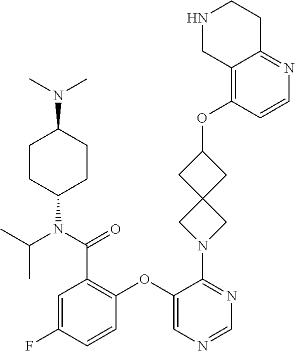 [SMILES: CC(C)N(C(=O)c1cc(F)ccc1Oc1cncnc1N1CC2(CC(Oc3ccnc4c3CNCC4)C2)C1)[C@H]1CC[C@H](N(C)C)CC1]

This compound was synthesized following the procedure described for the synthesis of Example 5, step 1, starting with 1 equivalent of tert-butyl ((1r,3r)-3-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)cyclobutyl)carbamate, except that 0.5 equivalent of Pd—C was used. After filtration and concentration, the crude was purified by Prep HPLC (Method B) to afford N-((1r,3r)-3-(dimethylamino)cyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a semi-solid.

Yield: 16.18%; ¹H NMR (400 MHz, DMSO-d₆): δ 8.29-8.25 (m, 1H), 8.16 (d, J=5.4 Hz, 1H), 7.76-7.63 (m, 1H), 7.31-7.22 (m, 1H), 7.16-7.05 (m, 2H), 6.66-6.61 (m, 1H), 4.73 (quin, J=6.4 Hz, 1H), 4.35-4.07 (m, 5H), 4.02-3.89 (m, 1H), 3.78-3.63 (m, 3H), 2.95 (t, J=5.8 Hz, 2H), 2.87-2.73 (m, 3H), 2.67 (br s, 2H), 2.29-2.19 (m, 3H), 2.14-2.07 (m, 2H), 2.04 (s, 3H), 1.95 (s, 3H), 1.88-1.77 (m, 1H), 1.49-0.89 (m, 6H); LCMS (Method J): Rt=1.84 min, m/z=616.0 [M+H]⁺; HPLC (Method A): Rt=4.15 min, 98.24%; Chiral SFC (Method AM): Rt=5.37 min, 100%.

### Example 12. N-(3-Aminobicyclo[1.1.1]pentan-1-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 12)

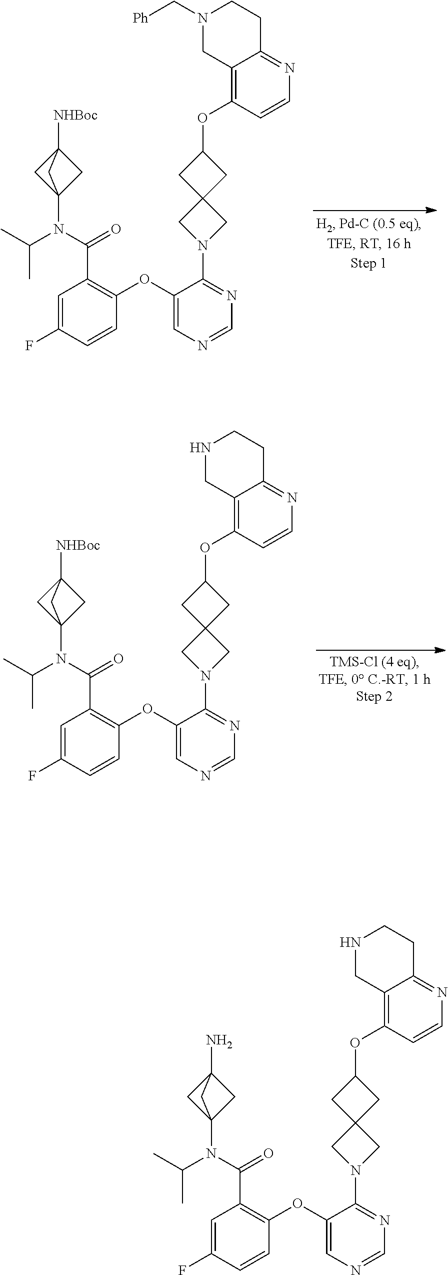 [SMILES: CC(C)N(C(=O)c1cc(F)ccc1Oc1cncnc1N1CC2(CC(Oc3ccnc4c3CN(Cc3ccccc3)CC4)C2)C1)C12CC(NC(=O)OC(C)(C)C)(C1)C2 ; CC(C)N(C(=O)c1cc(F)ccc1Oc1cncnc1N1CC2(CC(Oc3ccnc4c3CNCC4)C2)C1)C12CC(NC(=O)OC(C)(C)C)(C1)C2 ; CC(C)N(C(=O)c1cc(F)ccc1Oc1cncnc1N1CC2(CC(Oc3ccnc4c3CNCC4)C2)C1)C12CC(N)(C1)C2]

### Step 1. tert-Butyl (3-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)ox)benzamido)bicyclo[1.1.1]pentan-1-yl)carbamate

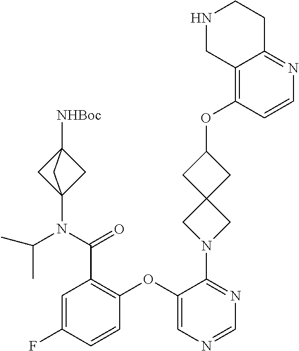 [SMILES: CC(C)N(C(=O)c1cc(F)ccc1Oc1cncnc1N1CC2(CC(Oc3ccnc4c3CNCC4)C2)C1)C12CC(NC(=O)OC(C)(C)C)(C1)C2]

This compound was synthesized following the procedure described for the synthesis of Example 5, step 1, starting with 1 equivalent of tert-butyl (3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)bicyclo[1.1.1]pentan-1-yl)carbamate, except that 0.5 equivalent of Pd—C was used. After filtration, the filtrate was concentrated under reduced pressure to obtain crude tert-butyl (3-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)bicyclo[1.1.1]pentan-1-yl)carbamate as a semi-solid. This material was used without further purification.

Yield: 91%; LCMS (Method A): Rt=1.79 min, m/z=700.4 [M+H]⁺, 97.74%.

### Step 2. N-(3-Aminobicyclo[1.1.1]pentan-1-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

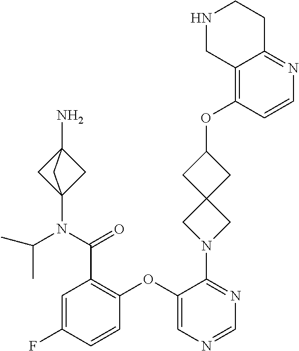 [SMILES: CC(C)N(C(=O)c1cc(F)ccc1Oc1cncnc1N1CC2(CC(Oc3ccnc4c3CNCC4)C2)C1)C12CC(N)(C1)C2]

This compound was synthesized following the procedure described for the synthesis of Example 5, step 2, starting with 1 equivalent of tert-butyl (3-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)bicyclo[1.1.1]pentan-1-yl)carbamate. After completion, the crude was purified by reverse phase chromatography using a SiliCycle SiliaSep C18 column (80 g) on Biotage isolera one (Mobile phase A: 10 mM NH₄HCO₃ in water, B: ACN) to afford N-(3-aminobicyclo[1.1.1]pentan-1-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 34.8%; ¹H NMR (400 MHz, DMSO-d₆): δ 8.27 (br s, 1H), 8.17 (d, J=5.6 Hz, 1H), 7.78-7.63 (m, 1H), 7.32-7.16 (m, 2H), 7.09-7.02 (m, 1H), 6.64 (d, J=5.6 Hz, 1H), 4.73 (quin, J=6.6 Hz, 1H), 4.32-4.10 (m, 4H), 4.06-3.97 (m, 1H), 3.70 (s, 2H), 2.95 (t, J=5.6 Hz, 2H), 2.86-2.73 (m, 3H), 2.71-2.65 (m, 2H), 2.30-2.16 (m, 6H), 1.78-1.61 (m, 4H), 1.38-1.06 (m, 6H); LCMS (Method L): Rt=1.76 min, m/z=600.2 [M+H]⁺; HPLC (Method A): Rt=4.02 min, 96.71%.

### Example 13. N-(3-(Dimethylamino)bicyclo[1.1.1]pentan-1-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 13)

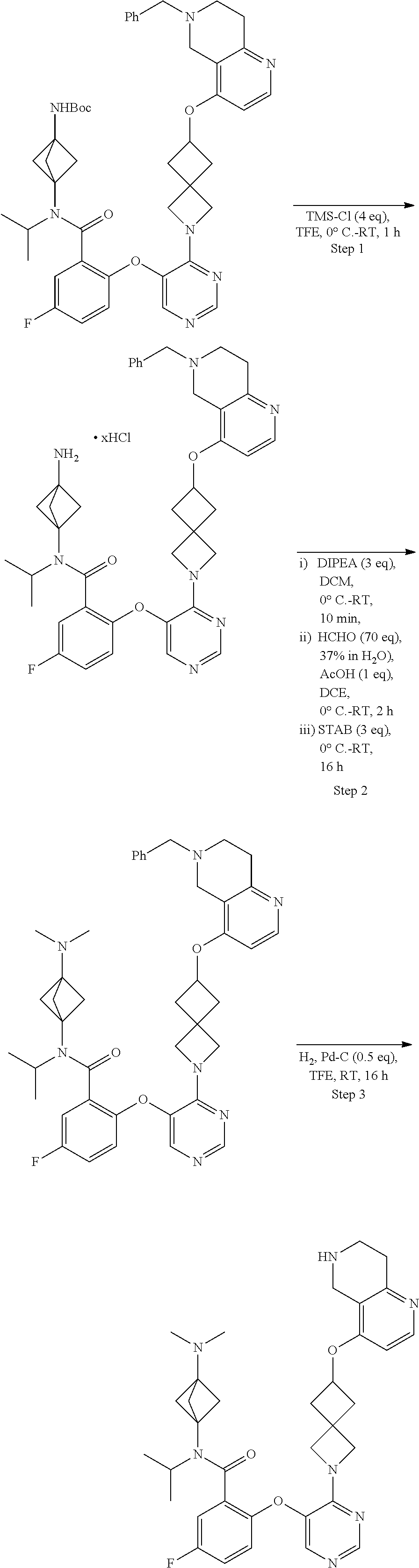

### Step 1. N-(3-Aminobicyclo[1.1.1]pentan-1-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl

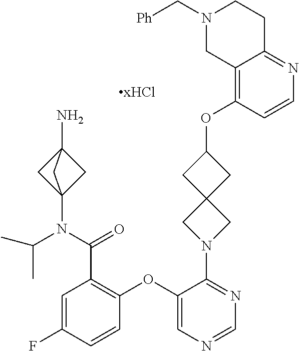 [SMILES: CC(C)N(C(=O)c1cc(F)ccc1Oc1cncnc1N1CC2(CC(Oc3ccnc4c3CN(Cc3ccccc3)CC4)C2)C1)C12CC(N)(C1)C2]

This compound was synthesized following the procedure described for the synthesis of Example 5, step 2, starting with 1 equivalent of tert-butyl (3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)bicyclo[1.1.1]pentan-1-yl)carbamate. After completion, the reaction was concentrated under reduced pressure to obtain crude N-(3-aminobicyclo[1.1.1]pentan-1-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl as a solid. This material was used without further purification.

Yield: 89%; LCMS (Method A): Rt=1.86 min, m/z=690.3 [M+H]⁺, 90.54%

### Step 2. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-N-(3-(dimethylamino)bicyclo[1.1.1]pentan-1-yl)-5-fluoro-N-isopropylbenzamide

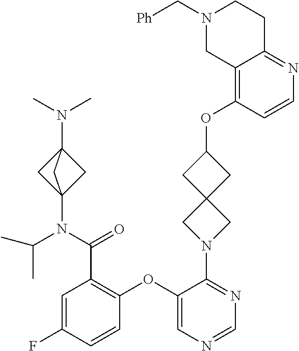 [SMILES: CC(C)N(C(=O)c1cc(F)ccc1Oc1cncnc1N1CC2(CC(Oc3ccnc4c3CN(Cc3ccccc3)CC4)C2)C1)C12CC(N(C)C)(C1)C2]

This compound was synthesized following the procedure described for the synthesis of Example 10, step 2, starting with 1 equivalent of N-(3-aminobicyclo[1.1.1]pentan-1-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl, except that 70 equivalents of formaldehyde (37% in H₂O), and 3 equivalents of STAB were used. After filtration and concentration, the crude was purified by reverse phase chromatography using a SiliCycle SiliaSep C18 column (80 g) on Biotage isolera one (Mobile phase A: 10 mM NH₄HCO₃ in water, B: ACN) to obtain 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-(3-(dimethylamino)bicyclo[1.1.1]pentan-1-yl)-5-fluoro-N-isopropylbenzamide as a solid.

Yield: 38.3%; LCMS (Method A): Rt=2.07 min, m/z=718.3 [M+H]⁺, 92.84%.

### Step 3. N-(3-(Dimethylamino)bicyclo[1.1.1]pentan-1-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

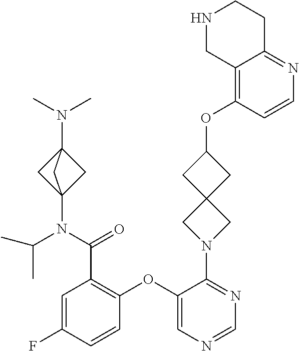 [SMILES: CC(C)N(C(=O)c1cc(F)ccc1Oc1cncnc1N1CC2(CC(Oc3ccnc4c3CNCC4)C2)C1)C12CC(N(C)C)(C1)C2]

This compound was synthesized following the procedure described for the synthesis of Example 5, step 1, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-(3-(dimethylamino)bicyclo[1.1.1]pentan-1-yl)-5-fluoro-N-isopropylbenzamide, except that 0.5 equivalent of Pd—C was used. After filtration and concentration, the crude was purified by Prep HPLC (Method B) to obtain N-(3-(dimethylamino)bicyclo[1.1.1]pentan-1-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 27.5%; ¹H NMR (400 MHz, DMSO-d₆): δ 8.29 (br s, 1H), 8.16 (d, J=5.6 Hz, 1H), 7.81-7.65 (m, 1H), 7.32-7.19 (m, 2H), 7.06-7.03 (m, 1H), 6.63 (d, J=5.6 Hz, 1H), 4.80-4.64 (m, 1H), 4.33-4.07 (m, 4H), 4.06-3.77 (m, 1H), 3.70 (s, 2H), 2.95 (t, J=5.8 Hz, 2H), 2.87-2.72 (m, 2H), 2.71-2.65 (m, 2H), 2.29-2.18 (m, 4H), 2.11 (br s, 3H), 2.01 (br s, 3H), 1.79-1.62 (m, 3H), 1.40-1.06 (m, 6H), two proton merged with solvent peaks; LCMS (Method H): Rt=3.10 min, m/z=628.3 [M+H]⁺; HPLC (Method A): Rt=4.55 min, 99.11%.

### Example 14. (S)-5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(pyrrolidin-3-yl)benzamide (Compound No. 14)

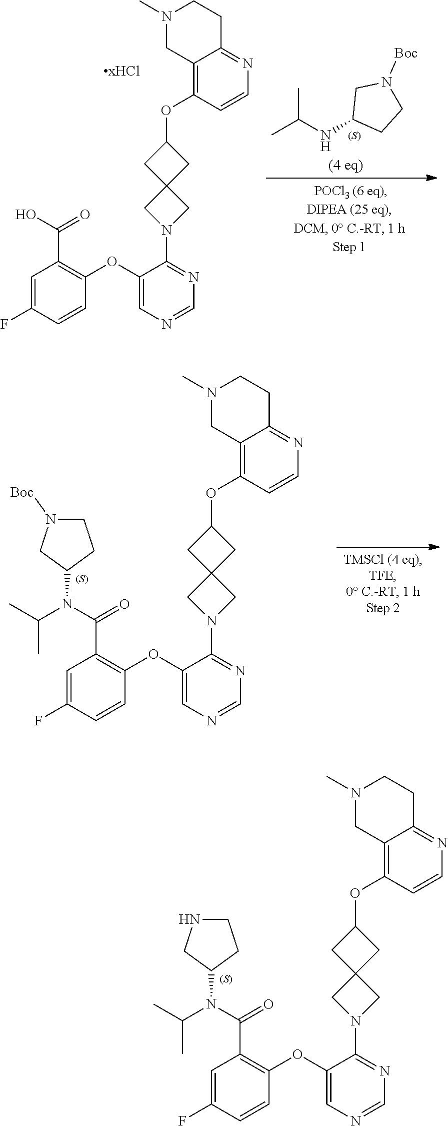

### Step 1. tert-Butyl (S)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)pyrrolidine-1-carboxylate

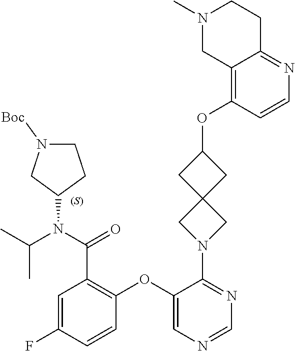

To a stirred solution of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid xHCl (350 mg, 0.712 mmol) in DCM (20 mL), tert-butyl (S)-3-(isopropylamino)pyrrolidine-1-carboxylate (650 mg, 2.85 mmol) and DIPEA (3.11 mL, 17.80 mmol) were added at 0° C. The reaction was stirred at RT for 5 min. To this reaction mixture, POCl₃ (0.398 mL, 4.27 mmol) was added at 0° C. and the reaction was stirred at RT for 1 h. The reaction progress was monitored by LCMS and TLC (100% EtOAc). After completion, the reaction was quenched with saturated bicarbonate solution (10 mL) and the mixture was extracted with DCM (2×10 mL). The combined organic layer was dried over sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain tert-butyl (S)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)pyrrolidine-1-carboxylate (60 mg, 11.97% yield) as a solid. LCMS (Method A): Rt=1.90 min, m/z=702.3 [M+H]⁺, 99.72%.

### Step 2. (S)—5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(pyrrolidin-3-yl)benzamide

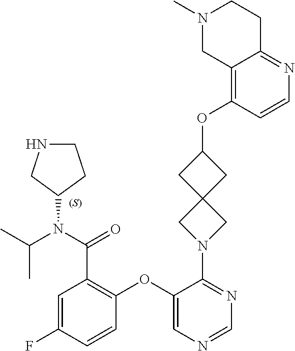

To a stirred solution of tert-butyl (S)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)pyrrolidine-1-carboxylate (60 mg, 0.085 mmol) in TFE (2 mL), TMSCl (0.044 mL, 0.342 mmol) was added at 0° C. The reaction was stirred at RT for 1 h. The reaction progress was monitored by TLC (10% MeOH in DCM). After completion, the reaction was concentrated under reduced pressure, and the residue was re-concentrated from EtOAc (5 mL). The crude was purified by reverse phase chromatography using a SiliCycle SiliaSep C18 column (40 g) on Biotage isolera one (Mobile phase A: 10 mM NH₄HCO₃ in water, B: ACN) to obtain (S)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(pyrrolidin-3-yl)benzamide (25 mg, 47.8% yield) as a solid. ¹H NMR (400 MHz, DMSO-d₆): δ 8.30-8.26 (m, 11H), 8.18 (d, J=5.6 Hz, 11H), 7.78-7.72 (m, 1H), 7.33-7.18 (m, 2H), 7.11-7.03 (m, 1H), 6.66 (br d, J=5.5 Hz, 1H), 4.75 (quin, J=6.6 Hz, 1H), 4.32-4.08 (m, 4H), 3.98-3.66 (m, 2H), 3.36 (br s, 2H), 3.18-3.06 (m, 1H), 2.94-2.73 (m, 6H), 2.63 (br t, J=5.8 Hz, 2H), 2.36 (s, 3H), 2.31-2.20 (m, 2H), 2.12-1.75 (m, 2H), 1.49-0.95 (m, 6H), two protons merged with solvent peaks; LCMS (Method A). Rt=1.63 min, m/z=602.3 [M+H]⁺; HPLC (Method A): Rt=4.35 min, 98.29%; Chiral SFC (Method P): Rt=6.64 min, 100%.

### Example 15. (R)-5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(pyrrolidin-3-yl)benzamide (Compound No. 15)

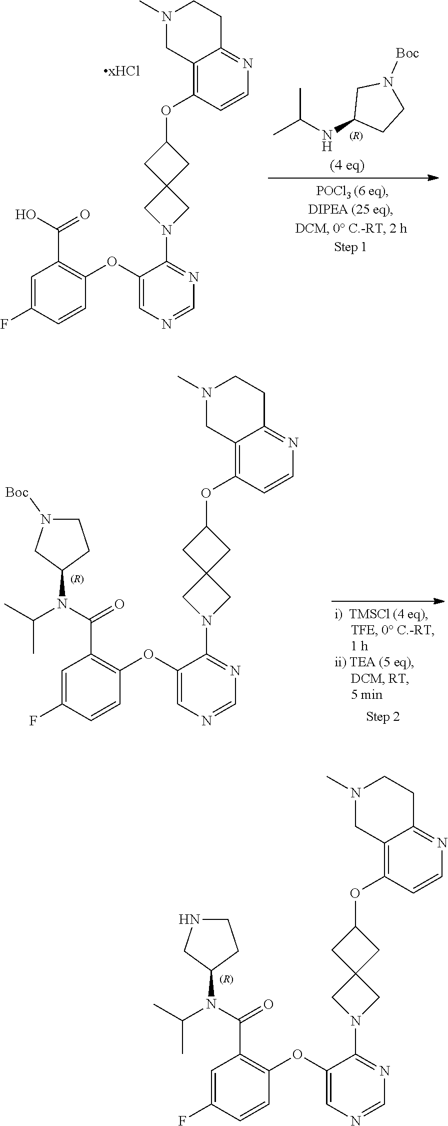

### Step 1. tert-Butyl (R)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)pyrrolidine-1-carboxylate

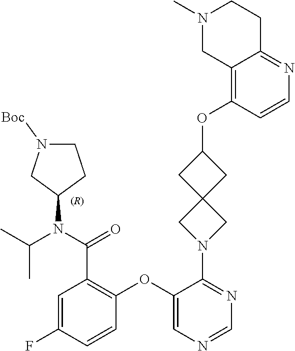

This compound was synthesized following the procedure described for the synthesis of Example 14, step 1, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid xHCl, except that 4 equivalents of tert-butyl (R)-3-(isopropylamino)pyrrolidine-1-carboxylate was used. The crude was purified by reverse phase chromatography using a SiliCycle SiliaSep C18 column (40 g) on Biotage isolera one (Mobile phase A: 10 mM NH₄HCO₃ in water, B: ACN) to obtain tert-butyl (R)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)pyrrolidine-1-carboxylates as a solid.

Yield: 12.37%; LCMS (Method A): Rt=1.90 min, m/z=702.3 [M+H]⁺, 88.32%.

### Step 2. (R)-5-Fluoro-N-isopropyl-2-((4-(6-((6-methyl-5,6,7,8-tetrahydro-1,6-naphthyridine-4-yl)oxy)-2-azaspiro[3.3]heptan-2-yl)pyrimidin-5-yl)oxy)-N-(pyrrolidin-3-yl)benzamide

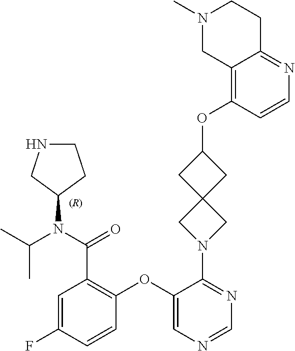

This compound was synthesized following the procedure described for the synthesis of Example 14, step 2, starting with 1 equivalent of tert-butyl (R)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)pyrrolidine-1-carboxylate. After completion, the reaction was concentrated under reduced pressure. The crude was dissolved in DCM, 5 equivalents of TEA was added, and the reaction was stirred at RT for 5 min. The reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain (R)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(pyrrolidin-3-yl)benzamide as a solid.

Yield: 46.4%; ¹H NMR (400 MHz, DMSO-d₆): δ 8.31-8.25 (m, 1H), 8.18 (d, J=5.5 Hz, 1H), 7.78-7.72 (m, 1H), 7.33-7.17 (m, 2H), 7.12-7.03 (m, 1H), 6.66 (br d, J=5.3 Hz, 1H), 4.81-4.66 (m, 1H), 4.33-4.09 (m, 4H), 3.96-3.65 (m, 2H), 3.36 (br s, 2H), 3.15-3.06 (m, 1H), 2.84-2.71 (m, 5H), 2.65-2.60 (m, 2H), 2.36 (s, 3H), 2.30-2.19 (m, 2H), 2.11-1.79 (m, 2H), 1.48-0.96 (m, 6H), three protons merged with solvent peaks; LCMS (Method A): Rt=1.55 min, m/z=602.3 [M+H]⁺; HPLC (Method A): Rt=4.36 min, 95.95%; Chiral SFC (Method P): Rt=6.67 min, 99.94%.

### Example 16. 5-Fluoro-N-isopropyl-N-(piperidin-4-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 16)

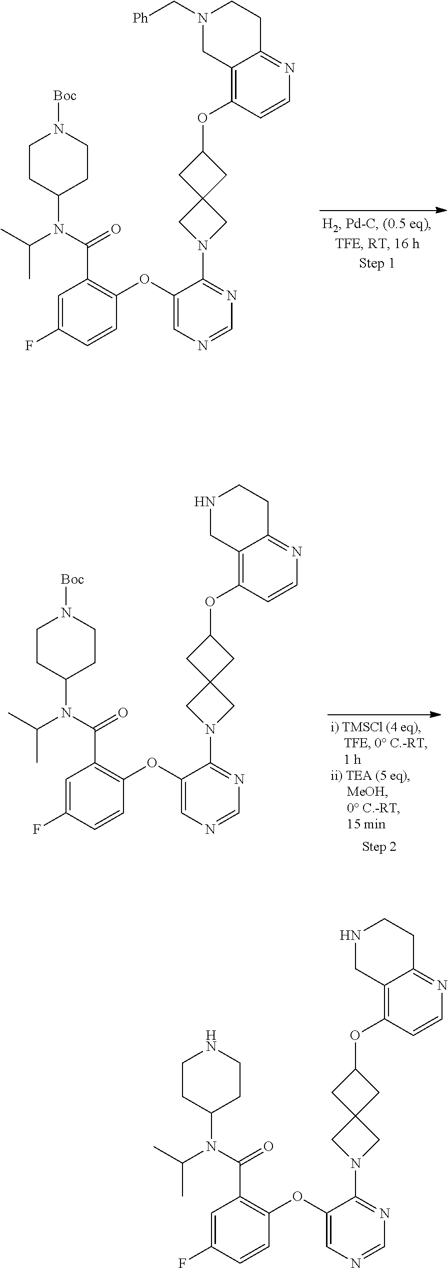 [SMILES: CC(C)N(C(=O)c1cc(F)ccc1Oc1cncnc1N1CC2(CC(Oc3ccnc4c3CN(Cc3ccccc3)CC4)C2)C1)C1CCN(C(=O)OC(C)(C)C)CC1 ; CC(C)N(C(=O)c1cc(F)ccc1Oc1cncnc1N1CC2(CC(Oc3ccnc4c3CNCC4)C2)C1)C1CCN(C(=O)OC(C)(C)C)CC1 ; CC(C)N(C(=O)c1cc(F)ccc1Oc1cncnc1N1CC2(CC(Oc3ccnc4c3CNCC4)C2)C1)C1CCNCC1]

### Step 1. tert-Butyl 4-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)piperidine-1-carboxylate

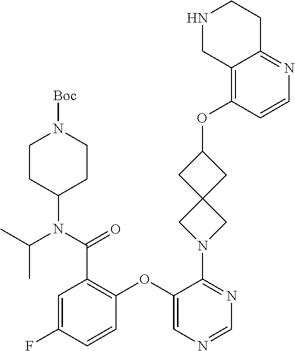 [SMILES: CC(C)N(C(=O)c1cc(F)ccc1Oc1cncnc1N1CC2(CC(Oc3ccnc4c3CNCC4)C2)C1)C1CCN(C(=O)OC(C)(C)C)CC1]

This compound was synthesized following the procedure described for the synthesis of Example 5, step 1, starting with 1 equivalent of tert-butyl 4-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)piperidine-1-carboxylate, except that 0.5 equivalent of Pd—C was used. After filtration, the reaction was concentrated under reduced pressure to afford crude tert-butyl 4-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)piperidine-1-carboxylate as a sticky solid. This material was used without further purification.

Yield: 84%; LCMS (Method A): Rt=1.88 min, m/z=702.2 [M+H]⁺, 93.23%.

### Step 2. 5-Fluoro-N-isopropyl-N-(piperidin-4-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

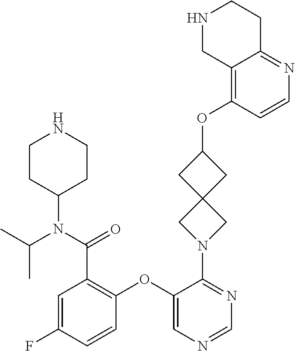 [SMILES: CC(C)N(C(=O)c1cc(F)ccc1Oc1cncnc1N1CC2(CC(Oc3ccnc4c3CNCC4)C2)C1)C1CCNCC1]

In a 25 mL round bottom flask, tert-butyl 4-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)piperidine-1-carboxylate (320 mg, 0.456 mmol) was dissolved in TFE (4 mL). To this solution, TMSCl (0.233 mL, 1.824 mmol) was added at 0° C. The reaction was stirred at RT for 1 h. The reaction was monitored by LCMS and TLC (10% MeOH in DCM). After completion, the reaction was concentrated under reduced pressure to afford crude 5-fluoro-N-isopropyl-N-(piperidin-4-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl (280 mg, 91% yield) as a solid. This material was used without further purification. LCMS (Method B): Rt=0.21 min, m/z=602.3 [M+H]⁺, 71.93%.

A portion of this material was neutralized to afford the free base. In a 25 mL round bottom flask, 5-fluoro-N-isopropyl-N-(piperidin-4-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl (130 mg, 0.204 mmol) was dissolved in in MeOH (2 mL), and TEA (0.142 mL, 1.019 mmol) was added at 0° C. The reaction was stirred at RT for 15 min, then was concentrated under reduced pressure. The crude compound was purified by reverse phase chromatography using a SiliCycle SiliaSep C18 column (60 g) on Biotage isolera one (Mobile phase A: 10 mM NH₄HCO₃ in water, B: ACN). The pure fractions were concentrated under reduced pressure and lyophilized to afford 5-fluoro-N-isopropyl-N-(piperidin-4-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (11.98 mg, 9.61%) as a solid. ¹H NMR (400 MHz, DMSO-d₆): δ 8.30-8.23 (m, 1H), 8.16 (d, J=5.5 Hz, 1H), 7.79-7.58 (m, 1H), 7.29-7.21 (m, 2H), 7.15-7.03 (m, 1H), 6.64 (d, J=5.6 Hz, 1H), 4.78-4.69 (m, 1H), 4.33-4.10 (m, 5H), 3.76-3.680 (m, 3H), 3.58-3.45 (m, 1H), 3.01-2.85 (m, 4H), 2.83-2.72 (m, 3H), 2.61-2.56 (m, 1H), 2.29-2.05 (m, 3H), 1.62-1.50 (m, 2H), 1.45-1.30 (m, 4H), 1.19-0.95 (m, 4H), two protons merged with solvent peaks; LCMS (Method H): Rt=0.94 min, m/z=602.3 [M+H]⁺; HPLC (Method A): Rt=3.88 min, 97.69%.

### Example 17. 5-Fluoro-N-isopropyl-N-(1-methylpiperidin-4-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 17)

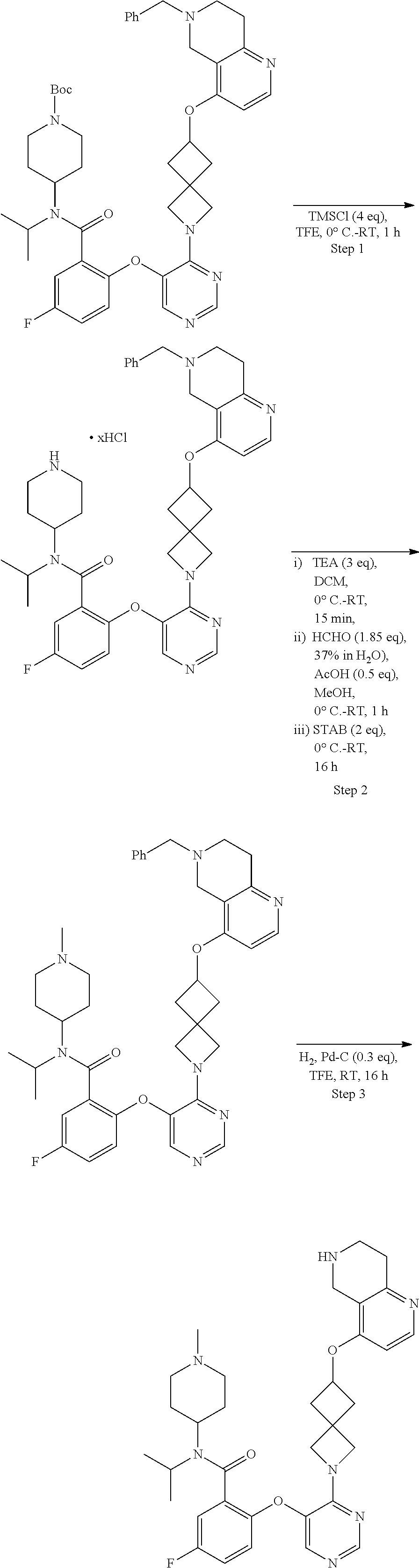

### Step 1. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(piperidin-4-yl)benzamide xHCl

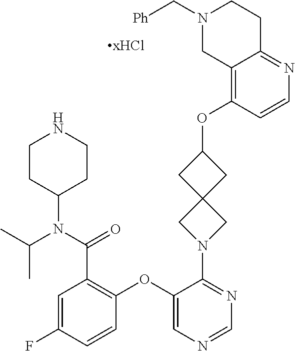 [SMILES: CC(C)N(C(=O)c1cc(F)ccc1Oc1cncnc1N1CC2(CC(Oc3ccnc4c3CN(Cc3ccccc3)CC4)C2)C1)C1CCNCC1]

This compound was synthesized following the procedure described for the synthesis of Example 5, step 2, starting with 1 equivalent of tert-butyl 4-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)piperidine-1-carboxylate. After completion, the reaction was concentrated under reduced pressure to afford crude 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(piperidin-4-yl)benzamide xHCl as a solid. This material was used without further purification

Yield: 81%; LCMS (Method A): Rt=2.05 min, m/z=692.3 [M+H]⁺, 72.42%.

### Step 2. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-methylpiperidin-4-yl)benzamide

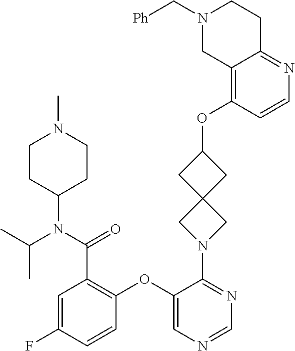 [SMILES: CC(C)N(C(=O)c1cc(F)ccc1Oc1cncnc1N1CC2(CC(Oc3ccnc4c3CN(Cc3ccccc3)CC4)C2)C1)C1CCN(C)CC1]

In a dried, 50 mL round bottom flask under a nitrogen atmosphere, 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(piperidin-4-yl)benzamide xHCl (360 mg, 0.494 mmol) was dissolved in DCM (4.00 mL), and TEA (0.207 mL, 1.483 mmol) was added at 0° C. The reaction was stirred at RT for 15 min. The reaction was concentrated under reduced pressure, and the residue was dissolved in MeOH (4 mL). To this solution, formaldehyde (0.068 mL, 0.914 mmol, 37% in H₂O) and AcOH (0.014 mL, 0.247 mmol) were added at 0° C., and the reaction was stirred at RT for 1 h. To this reaction mixture, sodium triacetoxyborohydride (210 mg, 0.989 mmol) was added at 0° C. The reaction was stirred at RT for 16 h. The reaction was monitored by TLC (10% MeOH in DCM). After completion, the reaction was concentrated under reduced pressure. Saturated sodium bicarbonate solution (10 mL) was added to the residue and the mixture was extracted with 10% MeOH in DCM (2×30 mL). The combined organic layer was dried over anhydrous sodium sulfate and filtered, and the filtrate was concentrated under reduced pressure. The crude compound was purified by reverse phase chromatography using a SiliCycle SiliaSep C18 column (60 g) on Biotage isolera one (Mobile phase A: 10 mM NH₄HCO₃ in water, B: ACN). The pure fractions were concentrated under reduced pressure to afford 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-methylpiperidin-4-yl)benzamide (140 mg, 40.0% yield) as a solid. ¹H NMR (400 MHz, DMSO-d₆): δ 8.29-8.25 (m, 1H), 8.19 (d, J=5.6 Hz, 1H), 7.76-7.61 (m, 1H), 7.37-7.32 (m, 4H), 7.30-7.21 (m, 3H), 7.15-7.03 (m, 1H), 6.66 (d, J=5.6 Hz, 1H), 4.72 (quin, J=6.4 Hz, 1H), 4.32-4.15 (m, 3H), 4.13-4.07 (m, 1H), 3.75-3.70 (m, 1H), 3.69 (s, 2H), 3.56-3.49 (m, 1H), 3.48 (s, 2H), 3.15-2.96 (m, 1H), 2.82-2.67 (m, 9H), 2.25-2.17 (m, 2H), 2.15-2.02 (m, 3H), 1.98-1.85 (m, 1H), 1.77-1.55 (m, 2H), 1.48-0.94 (m, 6H), one proton merged with solvent peaks; LCMS (Method A): Rt=2.03 min, m/z=706.3 [M+H]⁺, 99.71%.

### Step 3. 5-Fluoro-N-isopropyl-N-(1-methylpiperidin-4-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

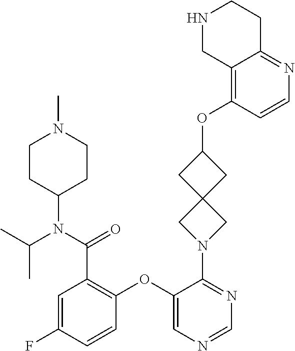 [SMILES: CC(C)N(C(=O)c1cc(F)ccc1Oc1cncnc1N1CC2(CC(Oc3ccnc4c3CNCC4)C2)C1)C1CCN(C)CC1]

This compound was synthesized following the procedure described for the synthesis of Example 5, step 1, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-methylpiperidin-4-yl)benzamide, except that 0.3 equivalent of Pd—C was used. After filtration and concentration, the crude was purified by reverse phase chromatography using a SiliCycle SiliaSep C18 column (60 g) on Biotage isolera one (Mobile phase A: 10 mM NH₄HCO₃ in water, B: ACN). The pure fractions were concentrated under reduced pressure and lyophilized to afford 5-fluoro-N-isopropyl-N-(1-methylpiperidin-4-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 54.2%; ¹H NMR (400 MHz, DMSO-d₆): δ 8.29-8.25 (m, 1H), 8.16 (d, J=5.5 Hz, 1H), 7.77-7.62 (m, 1H), 7.30-7.22 (m, 2H), 7.15-7.02 (m, 1H), 6.64 (d, J=5.8 Hz, 1H), 4.73 (quin, J=6.3 Hz, 1H), 4.36-4.07 (m, 4H), 3.76-3.71 (m, 1H), 3.70 (s, 2H), 3.58-3.45 (m, 1H), 3.15-3.01 (m, 1H), 2.95 (t, J=5.6 Hz, 2H), 2.84-2.60 (m, 9H), 2.29-2.19 (m, 2H), 2.16-2.01 (m, 3H), 1.98-1.81 (m, 1H), 1.76-1.58 (m, 2H), 1.49-0.91 (m, 6H); LCMS (Method H): Rt=2.97 min, m/z=616.2 [M+H]⁺; HPLC (Method A): Rt=4.18 min, 96.52%.

### Example 18. 5-Fluoro-N-isopropyl-N-(1-(oxetan-3-yl)azetidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 18)

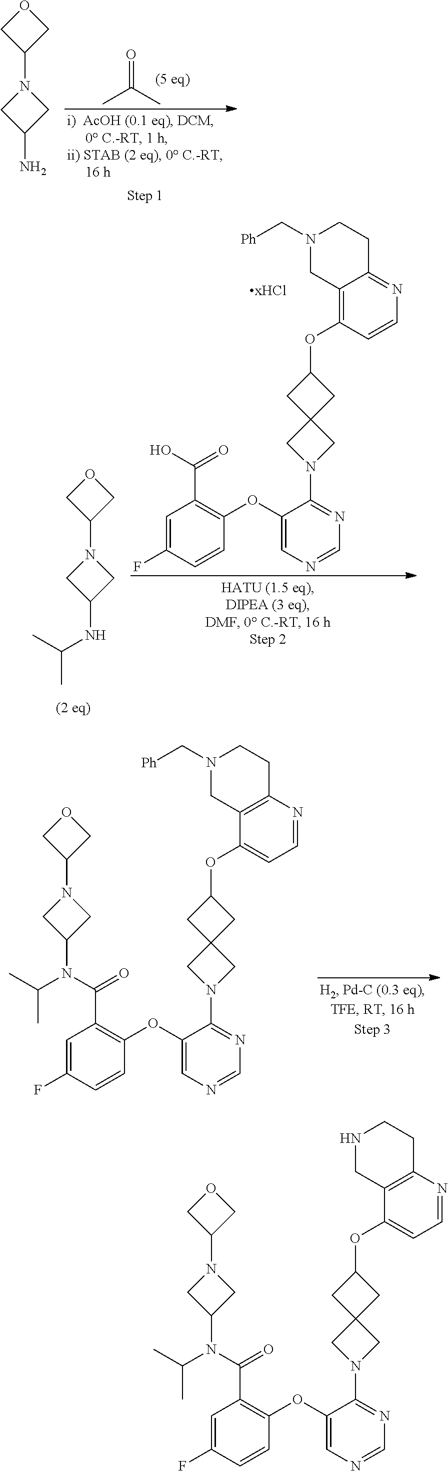

### Step 1. N-Isopropyl-1-(oxetan-3-yl)azetidin-3-amine

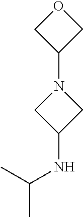

This compound was synthesized following the procedure described for the synthesis of Intermediate 1, starting with 1 equivalent of 1-(oxetan-3-yl)azetidin-3-amine, except that 5 equivalents of acetone and 0.1 equivalent of AcOH were used. After work-up, the filtrate was concentrated under reduced pressure to obtain crude N-isopropyl-1-(oxetan-3-yl)azetidin-3-amine (36.1% yield) as a sticky liquid. This material was used without further purification. ¹H NMR (400 MHz, DMSO-d₆): δ 4.52 (t, J=6.6 Hz, 2H), 4.35 (t, J=5.8 Hz, 2H), 3.69-3.61 (m, 1H), 3.48-3.41 (m, 2H), 3.40-3.34 (m, 1H), 3.29-3.25 (m, 1H), 2.79-2.61 (m, 3H), 0.93 (d, J=6.3 Hz, 6H).

### Step 2. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-(oxetan-3-yl)azetidin-3-yl)benzamide

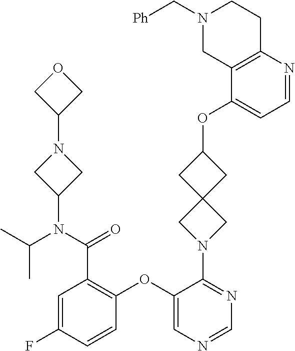 [SMILES: CC(C)N(C(=O)c1cc(F)ccc1Oc1cncnc1N1CC2(CC(Oc3ccnc4c3CN(Cc3ccccc3)CC4)C2)C1)C1CN(C2COC2)C1]

This compound was synthesized following the procedure described for the synthesis of Example 1, step 2, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid xHCl and 2 equivalents of N-isopropyl-1-(oxetan-3-yl)azetidin-3-amine. After work-up, the crude compound was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure to afford 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-(oxetan-3-yl)azetidin-3-yl)benzamide as a solid.

Yield: 25.2%; LCMS (Method A): Rt=1.86 min, m/z=720.2 [M+H]⁺, 99.32%.

### Step 3. 5-Fluoro-N-isopropyl-N-(1-(oxetan-3-yl)azetidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

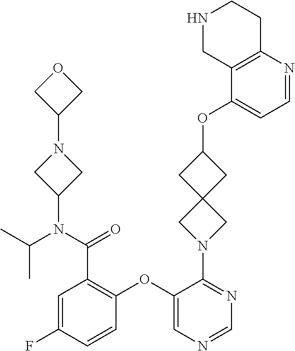 [SMILES: CC(C)N(C(=O)c1cc(F)ccc1Oc1cncnc1N1CC2(CC(Oc3ccnc4c3CNCC4)C2)C1)C1CN(C2COC2)C1]

This compound was synthesized following the procedure described for the synthesis of Example 5, step 1, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-(oxetan-3-yl)azetidin-3-yl)benzamide, except that 0.3 equivalent of Pd—C was used. After filtration and concentration, the crude compound was purified by reverse phase chromatography using a SiliCycle SiliaSep C18 column (60 g) on Biotage isolera one (Mobile phase A: 10 mM NH₄HCO₃ in water, B: ACN), then by Prep HPLC (Method B). The pure fractions were concentrated under reduced pressure and lyophilized to afford 5-fluoro-N-isopropyl-N-(1-(oxetan-3-yl)azetidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 19.76%; ¹H NMR (400 MHz, DMSO-d₆): δ 8.33-8.25 (m, 1H), 8.16 (d, J=5.6 Hz, 1H), 7.80-7.73 (m, 1H), 7.37-7.17 (m, 2H), 7.10-7.00 (m, 1H), 6.64 (d, J=5.6 Hz, 1H), 4.72 (quin, J=6.5 Hz, 1H), 4.59-4.42 (m, 3H), 4.34-4.06 (m, 7H), 4.04-3.82 (m, 2H), 3.76-3.66 (m, 3H), 3.49-3.43 (m, 1H), 3.28-3.20 (m, 1H), 3.12-3.06 (m, 1H), 2.95 (br t, J=5.8 Hz, 2H), 2.82-2.74 (m, 2H), 2.71-2.64 (m, 2H), 2.28-2.19 (m, 2H), 1.51-0.98 (m, 6H); LCMS (Method A): Rt=1.80 min, m/z=630.3 [M+H]⁺; HPLC (Method A): Rt=4.00 min, 99.92%.

### Example 19. N-(1-Cyclobutylazetidin-3-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 19)

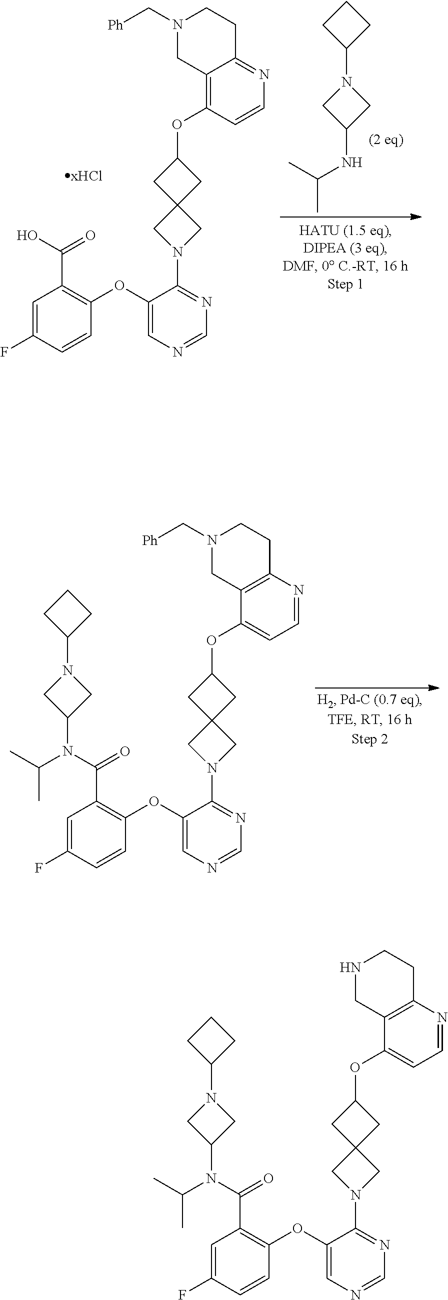 [SMILES: O=C(O)c1cc(F)ccc1Oc1cncnc1N1CC2(CC(Oc3ccnc4c3CN(Cc3ccccc3)CC4)C2)C1 ; CC(C)NC1CN(C2CCC2)C1 ; CC(C)N(C(=O)c1cc(F)ccc1Oc1cncnc1N1CC2(CC(Oc3ccnc4c3CN(Cc3ccccc3)CC4)C2)C1)C1CN(C2CCC2)C1 ; CC(C)N(C(=O)c1cc(F)ccc1Oc1cncnc1N1CC2(CC(Oc3ccnc4c3CNCC4)C2)C1)C1CN(C2CCC2)C1]

### Step 1. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-N-(1-cyclobutylazetidin-3-yl)-5-fluoro-N-isopropylbenzamide

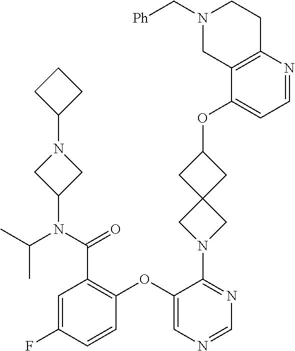 [SMILES: CC(C)N(C(=O)c1cc(F)ccc1Oc1cncnc1N1CC2(CC(Oc3ccnc4c3CN(Cc3ccccc3)CC4)C2)C1)C1CN(C2CCC2)C1]

This compound was synthesized following the procedure described for the synthesis of Example 1, step 2, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid xHCl and 2 equivalents of 1-cyclobutyl-N-isopropylazetidin-3-amine. After work-up, the crude compound was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure to afford 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-(1-cyclobutylazetidin-3-yl)-5-fluoro-N-isopropylbenzamide as a solid.

Yield: 20.98%; LCMS (Method B): Rt=1.20 min, m/z=718.2 [M+H]⁺, 86.56%.

### Step 2. N-(1-Cyclobutylazetidin-3-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

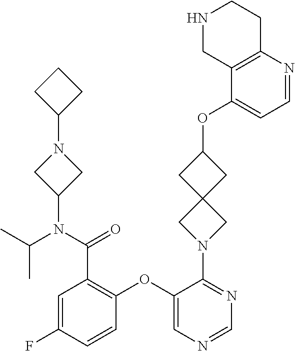 [SMILES: CC(C)N(C(=O)c1cc(F)ccc1Oc1cncnc1N1CC2(CC(Oc3ccnc4c3CNCC4)C2)C1)C1CN(C2CCC2)C1]

This compound was synthesized following the procedure described for the synthesis of Example 5, step 1, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-(1-cyclobutylazetidin-3-yl)-5-fluoro-N-isopropylbenzamide, except that 0.7 equivalent of Pd—C was used. After filtration and concentration, the crude compound was purified by reverse phase chromatography using a SiliCycle SiliaSep C18 column (40 g) on Biotage isolera one (Mobile phase A: 10 mM NH₄HCO₃ in water, B: ACN), then by Prep HPLC (Method B). The pure fractions were concentrated under reduced pressure and lyophilized to afford N-(1-cyclobutylazetidin-3-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 8.02%; ¹H NMR (400 MHz, DMSO-d₆): δ 8.32-8.23 (m, 1H), 8.16 (d, J=5.5 Hz, 1H), 7.82-7.67 (m, 1H), 7.35-7.15 (m, 2H), 7.12-7.00 (m, 1H), 6.64 (d, J=5.7 Hz, 1H), 4.77-4.69 (m, 1H), 4.35-3.97 (m, 6H), 3.84-3.64 (m, 3H), 3.22-3.03 (m, 2H), 2.99-2.93 (m, 2H), 2.34-2.72 (m, 2H), 2.71-2.66 (m, 3H), 2.24 (br dd, J=6.2, 12.4 Hz, 2H), 1.92-1.73 (m, 3H), 1.69-1.53 (m, 3H), 1.49-0.97 (m, 6H), two protons merged with solvent peaks; LCMS (Method H): Rt=1.00 min, m/z=628.4 [M+H]⁺; HPLC (Method B): Rt=2.63 min, 98.14%.

### Example 20. N-((1S,3S)-3-(Dimethyl amino)cyclopentyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 20)

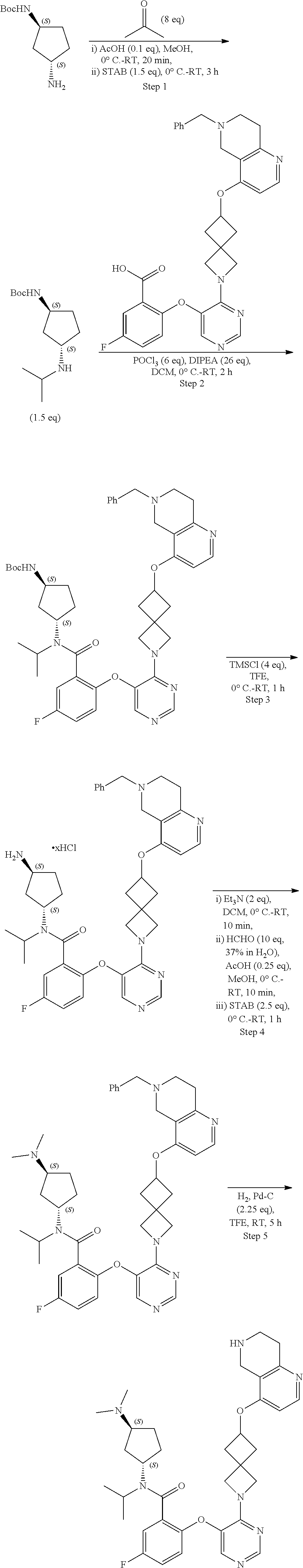

### Step 1. tert-Butyl ((1S,3S)-3-(isopropyl amino)cyclopentyl) carbamate

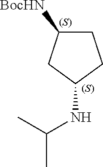

In a dried, 25 mL two neck round bottom flask under a nitrogen atmosphere, tert-butyl ((1S,3S)-3-aminocyclopentyl) carbamate (1.0 g, 4.99 mmol) was dissolved in MeOH (10 mL). To this solution, acetone (2.96 mL, 39.9 mmol) and AcOH (0.029 mL, 0.499 mmol) were added RT. The reaction was stirred at the RT for 20 min. To this reaction mixture, STAB (1.587 g, 7.49 mmol) was added at 0° C. and the reaction was stirred at RT for 3 h. The reaction progress was monitored by ¹H-NMR. After completion, the reaction was concentrated under reduced pressure. Water (50 mL) was added to the residue and the mixture was extracted with 10% MeOH in DCM (2×45 mL). The combined organic layer was dried over anhydrous sodium sulfate and filtered, and the filtrate was concentrated under reduced pressure to obtain crude tert-butyl ((1S,3S)-3-(isopropylamino)cyclopentyl)carbamate (1.2 g, quantitative yield). The crude was used for the next step without further purification. ¹H NMR (400 MHz, DMSO-d₆): δ 6.77 (br d, J=6.7 Hz, 1H), 3.90-3.75 (m, 1H), 3.24-3.11 (m, 1H), 2.76-2.69 (m, 1H), 1.94-1.76 (m, 2H), 1.61-1.47 (m, 2H), 1.37 (s, 9H), 1.34-1.26 (m, 1H), 1.23-1.12 (m, 2H), 0.94 (d, J=6.2 Hz, 6H).

### Step 2. tert-Butyl ((1S,3S)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclopentyl)carbamate

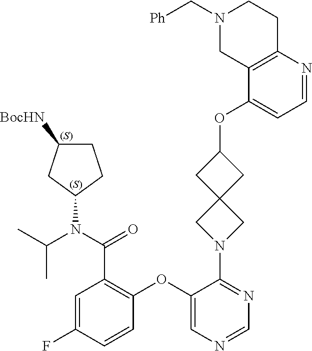

In a dried, 50 mL round bottom flask under a nitrogen atmosphere, 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid (700 mg, 1.233 mmol) and tert-butyl ((1S,3S)-3-(isopropylamino)cyclopentyl)carbamate (448 mg, 1.850 mmol) were dissolved in DCM (15 mL). To this reaction mixture, DIPEA (5.59 mL, 32.1 mmol)) and POCl₃ (0.692 mL, 7.40 mmol) were added at 0° C. and the reaction was stirred at RT for 2 h. The reaction progress was monitored by TLC (10% MeOH in DCM). After completion, the reaction was diluted with 10% NaHCO₃ solution (30 mL) and the mixture was extracted with DCM (3×45 mL). The combined organic layer was washed with brine solution (50 mL), dried over sodium sulfate and filtered, and the filtrate was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN) to obtain tert-butyl ((1S,3S)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclopentyl)carbamate (250 mg, 24.87% yield) as a solid. LCMS (Method A): Rt=2.24 min, m/z=792.4 [M+H]⁺; HPLC (Method A): Rt=7.03 min, 93.28%.

### Step 3. N-((1S,3S)-3-Aminocyclopentyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl

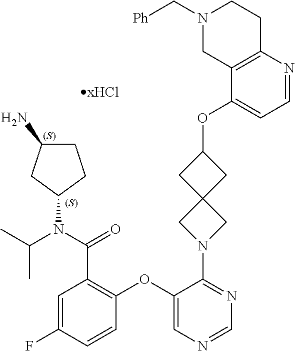

In a 25 mL two neck round bottom flask under a nitrogen atmosphere, tert-butyl ((1S,3S)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclopentyl)carbamate (250 mg, 0.316 mmol) was dissolved in TFE (3 mL). To this solution, TMSCl (0.161 mL, 1.263 mmol) was added at 0° C. and the reaction was stirred at RT for 1 h. The reaction progress was monitored by TLC (10% MeOH in DCM). After completion, the reaction was concentrated under reduced pressure and the residue was triturated with EtOAc (2×15 mL). The organic layer was decanted and the solid was dried under reduced pressure to obtain crude N-((1S,3S)-3-aminocyclopentyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl (215 mg, 90% yield) as a solid. This material was used without further purification. LCMS (Method A): Rt=2.04 min, m/z=692.3 [M+H]⁺, 83.94%.

### Step 4. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-N—((S,3S)-3-(dimethylamino)cyclopentyl)-5-fluoro-N-isopropylbenzamide

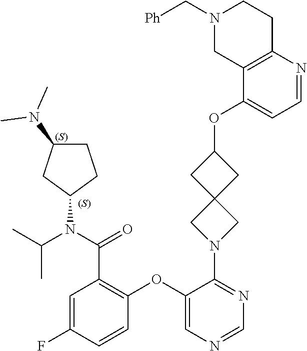

In a dried, 50 mL round bottom flask under a nitrogen atmosphere, N-((1S,3S)-3-aminocyclopentyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide hydrochloride (210 mg, 0.288 mmol) was dissolved in DCM (5 mL). To this solution, TEA (0.085 mL, 0.607 mmol) was added and the reaction was stirred RT for 10 min. The reaction was concentrated under reduced pressure. To this crude material, MeOH (5 mL) was added, and then formaldehyde (0.226 mL, 3.04 mmol, 37% in H₂O) and AcOH (4.34 μL, 0.076 mmol) were added at 0° C. The reaction was stirred at RT for 5 min. To this reaction mixture, STAB (161 mg, 0.759 mmol) was added at 0° C. and the reaction was stirred at RT for 1 h. The reaction progress was monitored by TLC (10% MeOH in DCM). After completion, the reaction was concentrated under reduced pressure. To this crude material, 10% NaHCO₃ solution (25 mL) was added and the mixture was extracted with 10% MeOH in DCM (3×45 mL). The combined organic layer was washed with brine solution (25 mL), dried over sodium sulfate, and filtered, and the filtrate was concentrated under reduced pressure. The crude was purified by reverse phase chromatography using a SiliCycle SiliaSep C18 column (50 g) on Biotage isolera one (Mobile phase A: 10 mM NH₄HCO₃ in water, B: ACN) to obtain 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1S,3S)-3-(dimethylamino)cyclopentyl)-5-fluoro-N-isopropylbenzamide (150 mg, 67.4% yield) as a solid. LCMS (Method A): Rt=2.20 min, m/z=720.3 [M+H]⁺, 98.15%.

### Step 5. N-((1S,3S)-3-(dimethylamino)cyclopentyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

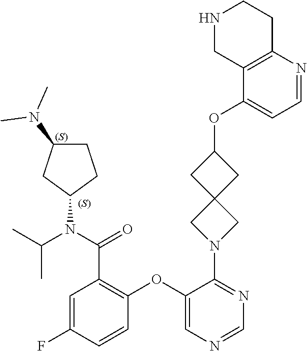

In a dried, 25 mL round bottom flask under a nitrogen atmosphere, 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1S,3S)-3-(dimethylamino)cyclopentyl)-5-fluoro-N-isopropylbenzamide (150 mg, 0.208 mmol) was dissolved in TFE (3 mL). To this solution, Pd—C (100 mg, 0.470 mmol) was added and the reaction was stirred at RT for 10 h under a hydrogen atmosphere (balloon pressure). The reaction progress was monitored by TLC (10% MeOH in DCM). After completion, the reaction was filtered through a Celite® pad, and the pad was washed with TFE (45 mL). The filtrate was concentrated under reduced pressure. The crude product was purified by reverse phase chromatography using a SiliCycle SiliaSep C18 column (40 g) on Biotage isolera one (Mobile phase A: 10 mM NH₄HCO₃ in water, B: ACN) to afford N-((1S,3S)-3-(dimethylamino)cyclopentyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (35 mg, 26.6% yield) as a solid. ¹H NMR (400 MHz, DMSO-d₆): δ 8.33-8.24 (m, 1H), 8.16 (d, J=5.5 Hz, 1H), 7.79-7.66 (m, 1H), 7.33-7.17 (m, 2H), 7.15-6.96 (m, 1H), 6.68-6.59 (m, 1H), 4.81-4.67 (m, 1H), 4.40-4.07 (m, 4H), 3.93-3.74 (m, 1H), 3.70 (s, 2H), 3.58-3.39 (m, 1H), 2.95 (br t, J=5.7 Hz, 2H), 2.87-2.73 (m, 3H), 2.71-2.66 (m, 2H), 2.29-2.18 (m, 3H), 2.14-1.99 (m, 6H), 1.97-1.86 (m, 1H), 1.81-1.52 (m, 3H), 1.48-0.93 (m, 8H); LCMS (Method C): Rt=1.15 min, m/z=630.3 [M+H]⁺; HPLC (Method A): Rt=4.08 min, 98.91%; Chiral SFC (Method Y): Rt=6.14 min, 99.49%.

### Example 21. N-((1R,3S)-3-(Dimethylamino)cyclopentyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 21)

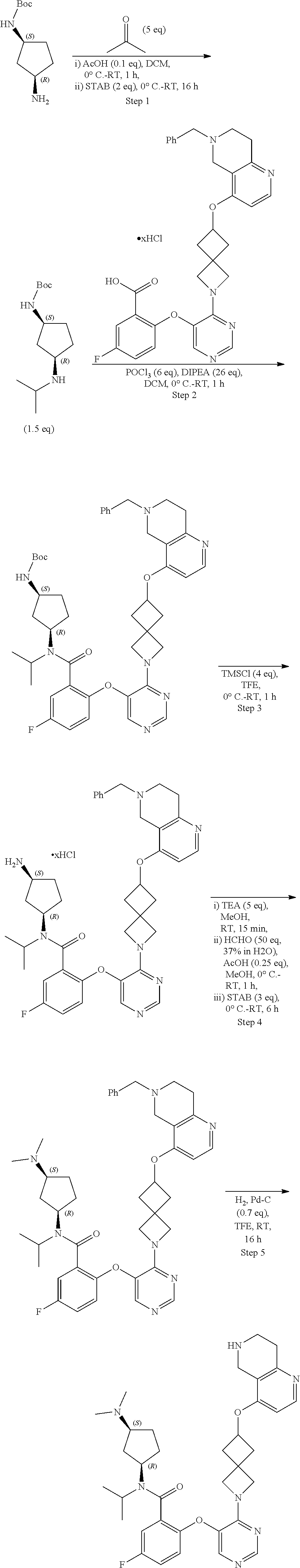

### Step 1. tert-Butyl ((1S,3R)-3-(isopropylamino)cyclopentyl)carbamate Boc

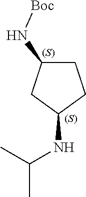

The reaction was performed following the same procedure in two batches.

This compound was synthesized following the procedure described for the synthesis of Example 20, step 1, starting with 1 equivalent of tert-butyl ((1S,3R)-3-aminocyclopentyl)carbamate, except that 5 equivalents of acetone and 2 equivalents of STAB were used, and DCM was used as the solvent. The reaction progress was monitored by TLC (10% MeOH in DCM). After completion, the two batches were combined and quenched with saturated NaHCO₃, and the mixture was extracted with DCM. The combined organic layer was dried over anhydrous sodium sulfate and filtered, and the filtrate was concentrated under reduced pressure to obtain crude tert-butyl ((1S,3R)-3-(isopropylamino)cyclopentyl)carbamate as a liquid. This material was used without further purification.

Combined yield. 99%: ¹H NMR (400 MHz, DMSO-d₆): δ 6.83 (br d, J=7.1 Hz, 1H), 3.77-3.67 (m, 1H), 3.13-3.03 (m, 1H), 2.82-2.70 (m, 1H), 2.08-1.98 (m, 1H), 1.75-1.65 (m, 2H), 1.53-1.41 (m, 1H), 1.37 (s, 9H), 1.21-1.10 (m, 1H), 0.97-0.95 (m, 6H), two protons merged with solvent peaks.

### Step 2. tert-Butyl ((1S,3R)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclopentyl)carbamate

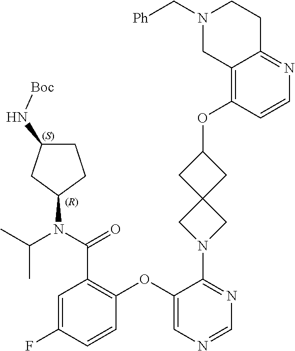

This compound was synthesized following the procedure described for the synthesis of Example 20, step 2, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid xHCl, except that 1.5 equivalents of tert-butyl ((1S,3R)-3-(isopropylamino)cyclopentyl)carbamate was used. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl ((1S,3R)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclopentyl)carbamate as a solid.

Yield: 33.1%; LCMS (Method B): Rt=1.50 min, m/z=792.2 [M+H]⁺, 92.29%.

### Step 3. N-((1R,3S)-3-Aminocyclopentyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl

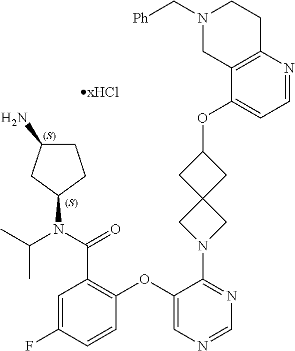

This compound was synthesized following the procedure described for the synthesis of Example 20, step 3, starting with 1 equivalent of tert-butyl ((1S,3R)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclopentyl)carbamate. After completion, the reaction was concentrated under reduced pressure to afford crude N-((1R,3S)-3-aminocyclopentyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl. This material was used without further purification.

Yield: 86%; LCMS (Method B): Rt=1.1 min, m/z=692.2 [M+H]⁺, 79.05%.

### Step 4. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-N-((1R,3S)-3-(dimethylamino)cyclopentyl)-5-fluoro-N-isopropylbenzamide

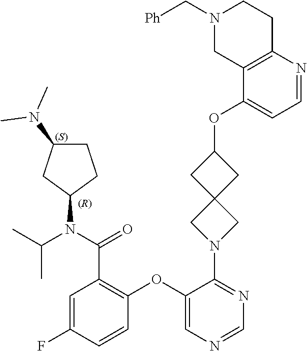

This compound was synthesized following the procedure described for the synthesis of Example 20, step 4, starting with 1 equivalent of N-((1R,3S)-3-aminocyclopentyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl, except that 50 equivalents of formaldehyde (37% in H₂O), 5 equivalents of TEA and 3 equivalents of STAB were used. After completion, the reaction was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1R,3S)-3-(dimethylamino)cyclopentyl)-5-fluoro-N-isopropylbenzamide as a solid.

Yield: 48.6%; LCMS (Method A): Rt=2.29 min, m/z=720.4 [M+H]⁺, 80.17%.

### Step 5. N-((1R,3S)-3-(Dimethylamino)cyclopentyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

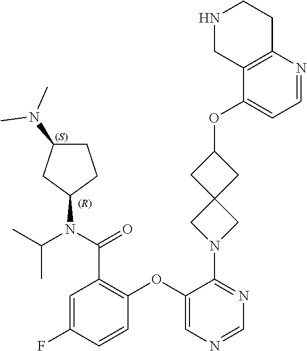

This compound was synthesized following the procedure described for the synthesis of Example 20, step 5, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1R,3S)-3-(dimethylamino)cyclopentyl)-5-fluoro-N-isopropylbenzamide, except that 0.7 equivalent of Pd—C was used. After completion, the reaction was filtered through a Celite® pad, and the pad was washed with MeOH. The filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain N-((1R,3S)-3-(dimethylamino)cyclopentyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 24.90%; H NMR (400 MHz, DMSO-d₆): δ 8.30-8.24 (m, 1H), 8.16 (d, J=5.6 Hz, 11H), 7.76-7.66 (m, 1H), 7.29-7.18 (m, 2H), 7.15-7.02 (m, 11H), 6.63 (br d, J=5.6 Hz, 1H), 4.73 (quin, J=6.4 Hz, 1H), 4.34-4.09 (m, 4H), 3.74-3.67 (m, 3H), 2.95 (br t, J=5.4 Hz, 2H), 2.84-2.73 (m, 3H), 2.70-2.66 (m, 2H), 2.42-2.36 (m, 1H), 2.29-2.19 (m, 3H), 2.16-2.00 (m, 7H), 1.89-1.58 (m, 4H), 1.49-0.95 (m, 6H), one proton merged with one peaks; LCMS (Method C): Rt=1.15 min, m/z=630.3 [M+H]⁺; HPLC (Method B): Rt=2.85 min, 96.09%; Chiral SFC (Method S): Rt=6.67 min, 99.94%.

### Example 22. N-((1R,3R)-3-(Dimethylamino)cyclopentyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 22)

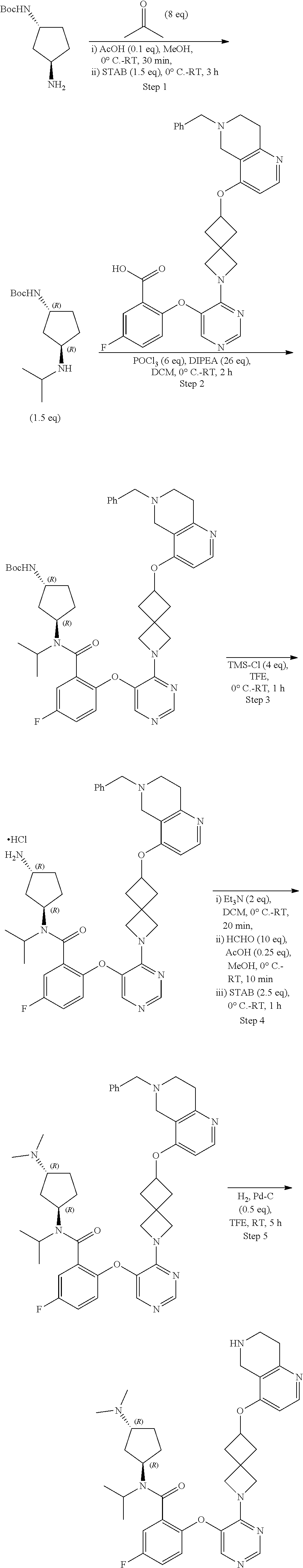

### Step 1. tert-Butyl ((1R,3R)-3-(isopropylamino)cyclopentyl)carbamate

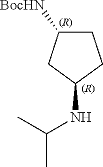

This compound was synthesized following the procedure described for the synthesis of Example 20, step 1, starting with 1 equivalent of tert-butyl ((1R,3R)-3-aminocyclopentyl)carbamate. After completion, the reaction was concentrated under reduced pressure. The crude was diluted with 10% aqueous NaHCO₃ solution, and the mixture was extracted with 10% MeOH in DCM. The combined organic layer was washed with brine solution, dried over anhydrous sodium sulfate, and filtered. The filtrate was concentrated under reduced pressure to obtain crude tert-butyl ((1R,3R)-3-(isopropylamino)cyclopentyl)carbamate as a liquid. This material was used without further purification.

Yield: 68.3%; ¹H NMR (400 MHz, DMSO-d₆): δ 6.82 (br d, J=7.3 Hz, 1H), 3.92-3.78 (m, 1H), 3.34-3.25 (m, 1H), 2.89-2.77 (m, 1H), 1.94-1.86 (m, 2H), 1.68-1.58 (m, 2H), 1.38 (s, 9H), 1.35-1.25 (m, 2H), 1.02 (d, J=6.3 Hz, 6H), one proton merged with solvent peaks.

### Step 2. tert-Butyl ((1R,3R)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclopentyl)carbamate

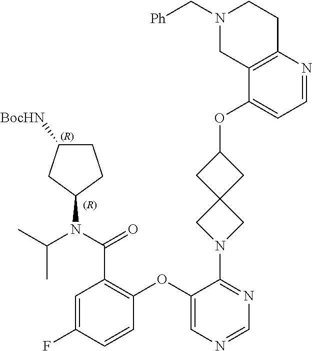

This compound was synthesized following the procedure described for the synthesis of Example 20, Step 2, starting with 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid (650 mg, 1.145 mmol), except that 1.5 equivalents of tert-butyl ((1R,3R)-3-(isopropylamino)cyclopentyl)carbamate was used. After work-up, the crude was purified by reverse phase chromatography using a SiliCycle SiliaSep C18 column (120 g cartridge) on Biotage isolera one (Mobile phase A: 10 mM NH₄HCO₃ in water, B: ACN) to afford tert-butyl ((1R,3R)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclopentyl)carbamate as a solid.

Yield: 33.1%; LCMS (Method A): Rt=2.26 min, m/z=792.4 [M+H]⁺, 99.90%.

### Step 3. N-((1R,3R)-3-Aminocyclopentyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl

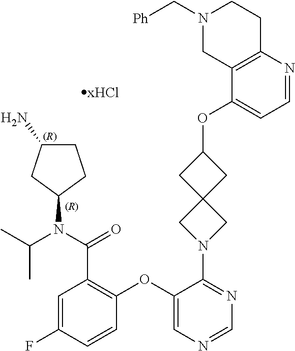

This compound was synthesized following the procedure described for the synthesis of Example 20, Step 3, starting with 1 equivalent of tert-butyl ((1R,3R)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclopentyl)carbamate to obtain crude N-((1R,3R)-3-aminocyclopentyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl as a solid. This material was used without further purification.

Yield: 79%; LCMS (Method A): Rt=1.97 min, m/z=692.2 [M+H]⁺, 79.65%.

### Step 4. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-N-((1R,3R)-3-(dimethylamino)cyclopentyl)-5-fluoro-N-isopropylbenzamide

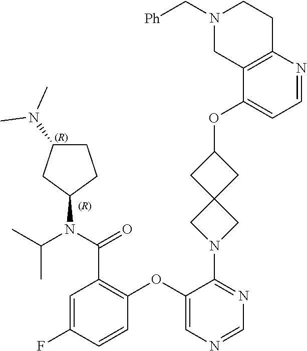

This compound was synthesized following the procedure described for the synthesis of Example 20, Step 4, starting with 1 equivalent of N-((1R,3R)-3-aminocyclopentyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide hydrochloride. After work-up, the crude was purified by reverse phase chromatography using a SiliCycle SiliaSep C18 column (50 g) on Biotage isolera one (Mobile phase A: 10 mM NH₄HCO₃ in water, B: ACN) to obtain 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1R,3R)-3-(dimethylamino)cyclopentyl)-5-fluoro-N-isopropylbenzamide as a solid.

Yield: 20.78%; LCMS (Method A): Rt=2.11 min, m/z=720.3 [M+H]⁺, 84.97%.

### Step 5. N-((1R,3R)-3-(Dimethylamino)cyclopentyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

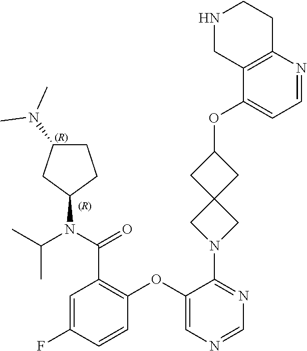

This compound was synthesized following the procedure described for the synthesis of Example 20, Step 5, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1R,3R)-3-(dimethylamino)cyclopentyl)-5-fluoro-N-isopropylbenzamide, except that 0.5 equivalent of Pd—C was used. After filtration and concentration, the crude product was purified by reverse phase chromatography using a SiliCycle SiliaSep C18 column (40 g) on Biotage isolera one (Mobile phase A: 10 mM NH₄HCO₃ in water, B: ACN) to obtain N-((1R,3R)-3-(dimethylamino)cyclopentyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 52.2%; ¹H NMR (400 MHz, DMSO-d₆): δ 8.32-8.23 (m, 1H), 8.16 (d, J=5.6 Hz, 1H), 7.79-7.64 (m, 1H), 7.39-6.97 (m, 3H), 6.68-6.58 (m, 1H), 4.79-4.67 (m, 1H), 4.36-4.07 (m, 4H), 3.80-3.58 (m, 4H), 2.95 (t, J=5.8 Hz, 2H), 2.87-2.72 (m, 3H), 2.70-2.64 (m, 2H), 2.28-2.17 (m, 3H), 2.14-1.99 (m, 6H), 1.98-1.81 (m, 1H), 1.80-1.50 (m, 3H), 1.50-0.94 (m, 7H), one proton merged with solvent peaks; LCMS (Method C): Rt=1.15 min, m/z=630.3 [M+H]⁺; HPLC (Method A): Rt=4.09 min, 99.93%.

### Example 23. N-((1S,3R)-3-(Dimethylamino)cyclopentyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 23)

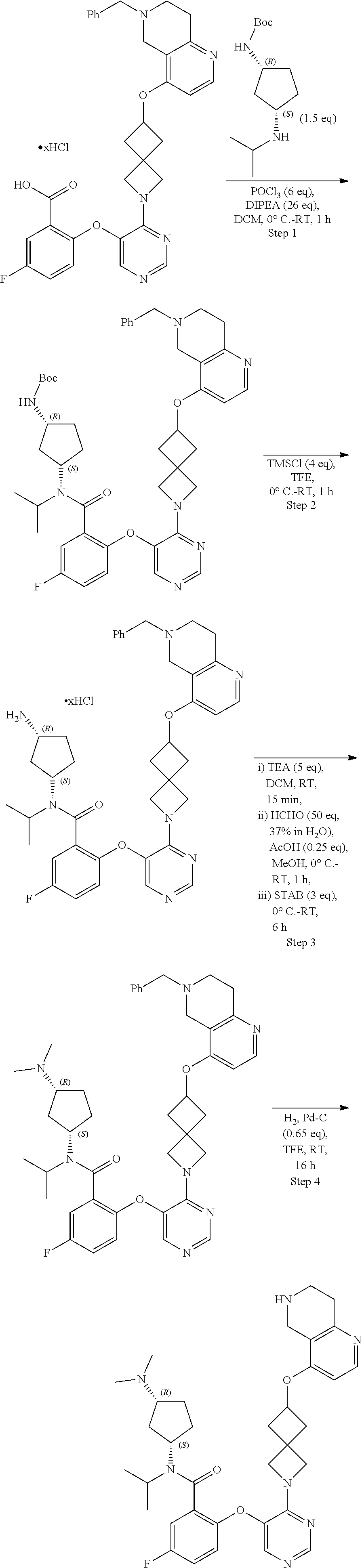

### Step 1. tert-Butyl ((1R,3S)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclopentyl)carbamate

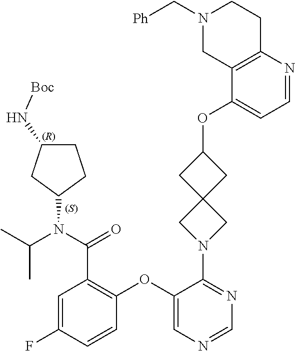

This compound was synthesized following the procedure described for the synthesis of Example 20, step 2, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid xHCl and 1.5 equivalents of tert-butyl ((1R,3S)-3-(isopropylamino)cyclopentyl)carbamate. After work-up, the crude was purified by reverse phase chromatography using a SiliCycle SiliaSep C18 column (40 g) on Biotage isolera one (Mobile phase A: 10 mM NH₄HCO₃ in water, B: ACN) to obtain tert-butyl ((1R,3S)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclopentyl)carbamate as a solid.

Yield: 28.7%; LCMS (Method B): Rt=1.53 min, m/z=792.2 [M+H]⁺, 97.41%.

### Step 2. N-((1S,3R)-3-Aminocyclopentyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl

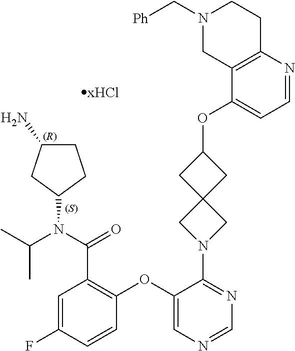

This compound was synthesized following the procedure described for the synthesis of Example 20, step 3, starting with 1 equivalent of tert-butyl ((1R,3S)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclopentyl)carbamate. After completion, the reaction was concentrated under reduced pressure to afford crude N-((1S,3R)-3-aminocyclopentyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl. This material was used without further purification.

Yield: 79%; LCMS (Method A): Rt=2.07 min, m/z=692.2 [M+H]⁺, 60.43%.

### Step 3. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-N—((S,3R)-3-(dimethylamino)cyclopentyl)-5-fluoro-N-isopropylbenzamide

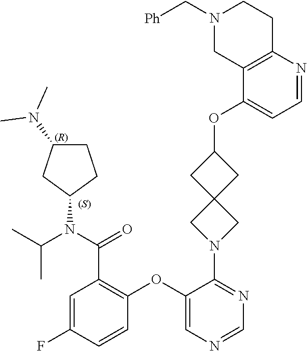

This compound was synthesized following the procedure described for the synthesis of Example 20, step 4, starting with 1 equivalent of N-((1S,3R)-3-aminocyclopentyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl, except that 50 equivalents of formaldehyde (37% in H₂O), 5 equivalents of TEA and 3 equivalents of STAB were used. After completion, the reaction was concentrated under reduced pressure. The crude was purified by reverse phase chromatography using a SiliCycle SiliaSep C18 column (60 g) on Biotage isolera one (Mobile phase A: 10 mM NH₄HCO₃ in water, B: ACN). The pure fractions were concentrated under reduced pressure to afford 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1S,3R)-3-(dimethylamino)cyclopentyl)-5-fluoro-N-isopropylbenzamide as a sticky liquid.

Yield: 51.1%; LCMS (Method A): Rt=2.19 min, m/z=720.2 [M+H]⁺, 94.72%.

### Step 4. N-((1S,3R)-3-(Dimethylamino)cyclopentyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

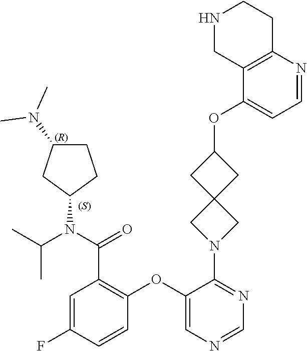

This compound was synthesized following the procedure described for the synthesis of Example 20, step 5, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1S,3R)-3-(dimethylamino)cyclopentyl)-5-fluoro-N-isopropylbenzamide, except that 0.65 equivalent of Pd—C was used. After completion, the reaction was filtered through a Celite® pad, and the pad was washed with MeOH. The filtrate was concentrated under reduced pressure. The crude was purified by Prep HPLC (Method G). The fractions were concentrated under reduced pressure. The residue was dissolved in 10% NaOH, and the mixture was extracted with 10% MeOH in DCM. The combined organic layer was dried over anhydrous sodium sulfate and filtered, and the filtrate was concentrated under reduced pressure. The crude was re-purified by reverse phase chromatography using a SiliCycle SiliaSep C18 column (12 g) on Biotage isolera one (Mobile phase A: 10 mM NH₄HCO₃ in water, B: ACN). The pure fractions were concentrated under reduced pressure and lyophilized to afford N-((1S,3R)-3-(dimethylamino)cyclopentyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 16.89%; ¹H NMR (400 MHz, DMSO-d₆): δ 8.29-8.24 (m, 1H), 8.19-8.11 (m, 1H), 7.76-7.66 (in, 1H), 7.28-7.21 (m, 2H), 7.11-7.03 (m, 1H), 6.63 (d, J=5.6 Hz, 1H), 4.78-4.68 (m, 1H), 4.35-4.10 (m, 4H), 3.74-3.65 (m, 3H), 3.56-3.46 (m, 2H), 2.99-2.91 (m, 2H), 2.85-2.74 (m, 2H), 2.67-2.60 (m, 11H), 2.42-2.36 (m, 11H), 2.29-2.17 (m, 3H), 2.17 −2.02 (in, 7H), 1.85-1.60 (m, 4H), 1.48-0.96 (in, 6H), one proton merged with solvent peaks; LCMS (Method C): Rt=1.48 min, m/z=630.3 [M+H]⁺; HPLC (Method A). Rt=4.25 min, 98.31%; Chiral SFC (Method U): Rt=2.72 min, 98.52%.

### Example 24. N-((1S,3S)-3-(Dimethylamino)cyclopentyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 24)

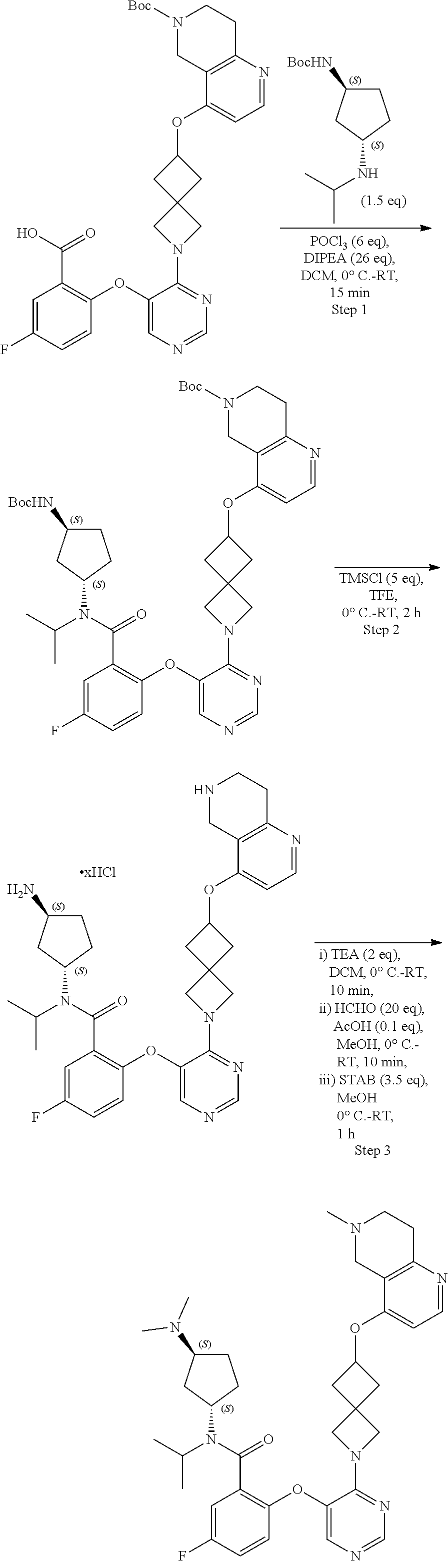

### Step 1. tert-Butyl 4-((2-(5-(2-(((1S,3S)-3-((tert-butoxycarbonyl)amino)cyclopentyl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate

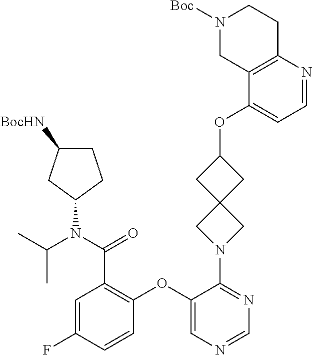 [SMILES: CC(C)N(C(=O)c1cc(F)ccc1Oc1cncnc1N1CC2(CC(Oc3ccnc4c3CN(C(=O)OC(C)(C)C)CC4)C2)C1)[C@H]1CC[C@H](NC(=O)OC(C)(C)C)C1]

This compound was synthesized following the procedure described for the synthesis of Example 20, Step 2, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.5 equivalents of tert-butyl ((1S,3S)-3-(isopropylamino)cyclopentyl)carbamate. After work-up, the crude product was purified by reverse phase chromatography using a SiliCycle SiliaSep C18 column (80 g) on Biotage isolera one (Mobile phase A: 10 mM NH₄HCO₃ in water, B: ACN) to obtain tert-butyl 4-((2-(5-(2-(((1S,3S)-3-((tert-butoxycarbonyl)amino)cyclopentyl)xisopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate as a solid.

Yield: 47.3% yield; ¹H NMR (400 MHz, DMSO-d₆): δ 8.31-8.26 (m, 1H), 8.23 (br d, J=5.6 Hz, 1H), 7.80-7.70 (m, 1H), 7.30-7.20 (m, 2H), 7.10-7.00 (m, 1H), 6.88-6.76 (m, 1H), 6.74 (d, J=5.5 Hz, 1H), 4.85-4.72 (m, 1H), 4.42-4.35 (m, 2H), 4.33-4.28 (m, 1H), 4.25-4.20 (m, 1H), 4.17-4.11 (m, 1H), 3.91-3.79 (m, 1H), 3.73-3.65 (m, 1H), 3.61 (br s, 2H), 3.45-3.37 (m, 2H), 2.87-2.76 (m, 4H), 2.32-2.23 (m, 2H), 2.21-2.11 (m, 1H), 2.09-1.95 (m, 2H), 1.89-1.78 (m, 1H), 1.43 (s, 9H), 1.40-1.32 (m, 9H), 1.29-0.88 (m, 8H); LCMS (Method A): Rt=2.24 min, m/z=802.3 [M+H]⁺, 87.51%.

### Step 2. N-((1S,3S)-3-Aminocyclopentyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl

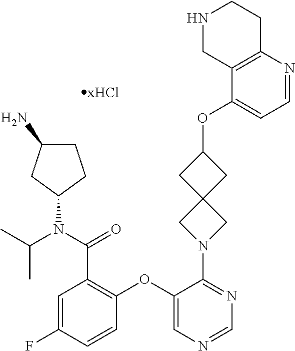 [SMILES: CC(C)N(C(=O)c1cc(F)ccc1Oc1cncnc1N1CC2(CC(Oc3ccnc4c3CNCC4)C2)C1)[C@H]1CC[C@H](N)C1]

This compound was synthesized following the procedure described for the synthesis of Example 20, Step 3, starting with 1 equivalent of tert-butyl 4-((2-(5-(2-(((1S,3S)-3-((tert-butoxycarbonyl)amino)cyclopentyl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate, except that 5 equivalents of TMSCl was used. After completion, the reaction was concentrated under reduced pressure to obtain crude N-((1S,3S)-3-aminocyclopentyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl as a gummy liquid. This material was used without further purification.

Yield: 90%; LCMS (Method H): Rt=1.60 min, m/z=602.5 [M+H]⁺, 67.96%.

### Step 3. N-((1S,3S)-3-(Dimethylamino)cyclopentyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxybenzamide

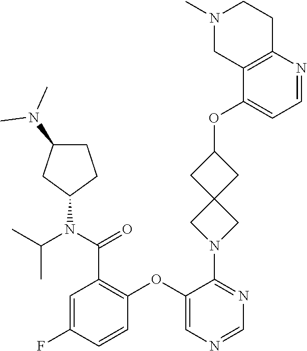 [SMILES: CC(C)N(C(=O)c1cc(F)ccc1Oc1cncnc1N1CC2(CC(Oc3ccnc4c3CN(C)CC4)C2)C1)[C@H]1CC[C@H](N(C)C)C1]

This compound was synthesized following the procedure described for the synthesis of Example 20, Step 4, starting with 1 equivalent of N-((1S,3S)-3-aminocyclopentyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl, except that 20 equivalents of formaldehyde (37% in H₂O), 0.1 equivalent of AcOH, and 3.5 equivalents of STAB were used. After work-up, the crude product was purified by reverse phase chromatography using a SiliCycle SiliaSep C18 column (80 g) on Biotage isolera one (Mobile phase A: 10 mM NH₄HCO₃ in water, B: ACN) to obtain N-((1S,3S)-3-(dimethylamino)cyclopentyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 9.32%; ¹H NMR (400 MHz, DMSO-d₆): δ 8.31-8.25 (m, 1H), 8.18 (d, J=5.5 Hz, 1H), 7.81-7.67 (m, 1H), 7.34-7.17 (m, 2H), 7.16-7.00 (m, 1H), 6.71-6.61 (m, 1H), 4.87-4.65 (m, 1H), 4.39-4.07 (m, 4H), 3.92-3.63 (m, 2H), 3.37 (br s, 2H), 2.86-2.73 (m, 5H), 2.63 (br t, J=5.8 Hz, 2H), 2.37 (s, 3H), 2.34-2.32 (m, 1H), 2.30-2.19 (m, 3H), 2.13-1.99 (m, 6H), 1.96-1.86 (m, 1H), 1.80-1.55 (m, 2H), 1.50-1.29 (m, 2H), 1.13-0.93 (m, 3H), one proton merged with solvent peaks; LCMS (Method H): Rt=0.95 min, m/z=644.3 [M+H]⁺; HPLC (Method A): Rt=4.57 min, 99.03%; Chiral SFC (Method D): Rt=1.88 min, 100%.

### Example 25. 5-Fluoro-N-((3R,4R)-4-fluoro-1-methylpyrrolidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 25)

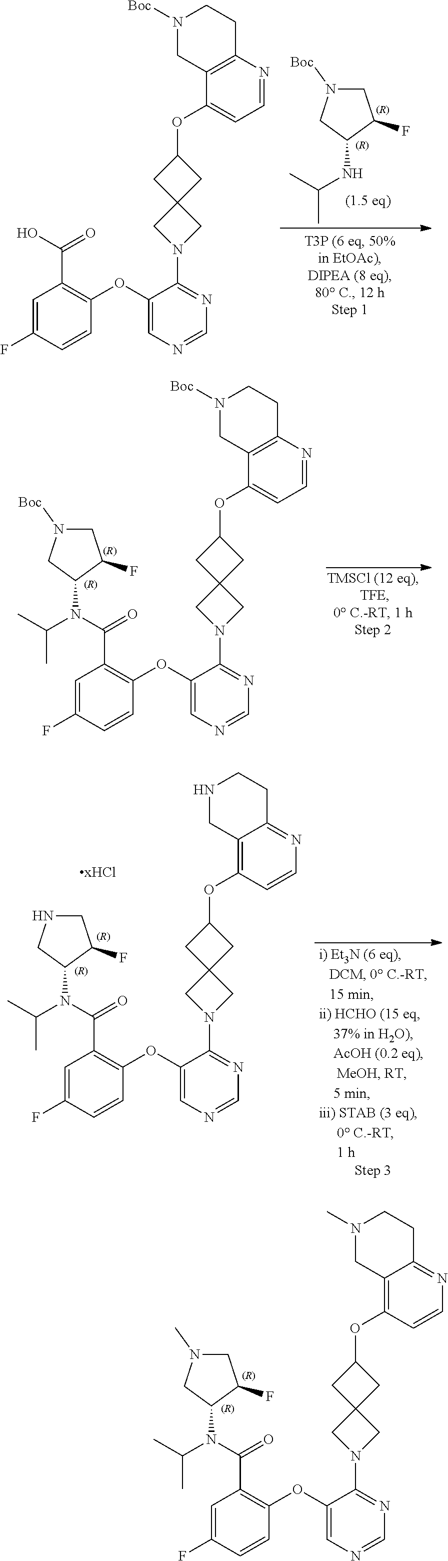

### Step 1. tert-Butyl 4-((2-(S-(2-(((3R,4R)-1-(tert-butoxycarbonyl)-4-fluoropyrrolidin-3-yl) (isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate

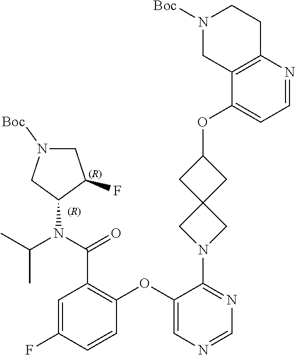

In an 8 mL vial, 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid (300 mg, 0.519 mmol) and tert-butyl (3R,4R)-3-fluoro-4-(isopropyl amino)pyrrolidine-1-carboxylate (192 mg, 0.779 mmol) were dissolved in DIPEA (724 μL, 4.16 mmol) at RT. T3P (1983 mg, 3.12 mmol, 50% in EtOAc) was added at RT and the mixture was stirred at 80° C. for 12 h. The reaction progress was monitored by TLC (10% MeOH in DCM). The reaction was quenched with aqueous Na₂CO₃ solution (10 mL), ice-cold water (10 mL) was added, and the mixture was extracted with EtOAc (2×30 mL). The combined organic layer was washed with brine (10 mL), dried over sodium sulfate and filtered, and the filtrate was concentrated under reduced pressure. The crude product was purified by reverse phase chromatography using a SiliCycle SiliaSep C18 column (80 g) on Biotage isolera one (Mobile phase A: 10 mM NH₄HCO₃ in water, B: ACN) to obtain tert-butyl 4-((2-(5-(2-(((3R,4R)-1-(tert-butoxycarbonyl)-4-fluoropyrrolidin-3-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate (150 mg, 35.3% yield) as a solid. ¹H NMR (400 MHz, DMSO-d₆): δ 8.33-8.28 (m, 1H), 8.25-8.19 (m, 1H), 7.83-7.73 (m, 1H), 7.50-7.42 (m, 1H), 7.33-7.25 (m, 1H), 7.14-7.04 (m, 1H), 6.77-6.70 (m, 1H), 5.72-5.24 (m, 1H), 4.83-4.72 (m, 1H), 4.36 (br s, 2H), 4.33-4.05 (m, 5H), 3.86-3.72 (m, 2H), 3.61 (br t, J=5.8 Hz, 2H), 3.57-3.44 (m, 1H), 2.86-2.73 (m, 4H), 2.32-2.22 (m, 2H), 1.47-1.34 (m, 18H), 1.18-0.93 (m, 6H), two protons merged with solvent peaks; LCMS (Method A): Rt=2.29 min, m/z=806.2 [M+H]⁺, 98.49%.

### Step 2. 5-Fluoro-N-((3R,4R)-4-fluoropyrrolidin-3-yl)-N-isopropyl-2-((4-(6-((5,6,7,8-tetrahydro-1,6-naphthyridin-4-yl)oxy)-2-azaspiro[3.3]heptan-2-yl)pyrimidin-S-yl)oxy)benzamide

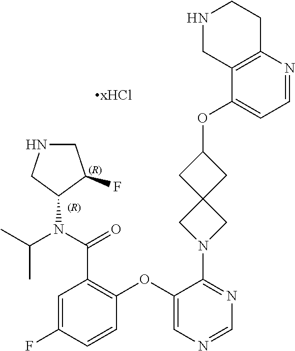

This compound was synthesized following the procedure described for the synthesis of Example 20, Step 3, starting with 1 equivalent tert-butyl 4-((2-(5-(2-(((3R,4R)-1-(tert-butoxycarbonyl)-4-fluoropyrrolidin-3-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate, except that 12 equivalents of TMSCl was used. After completion, the reaction was concentrated under reduced pressure to obtain crude 5-fluoro-N-((3R,4R)-4-fluoropyrrolidin-3-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl as a solid. This material was used without further purification.

Yield: 79%; LCMS (Method A): Rt=1.51 min, m/z=606.2 [M+H]⁺, 82.78%.

### Step 3. 5-Fluoro-N-((3R,4R)-4-fluoro-1-methylpyrrolidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

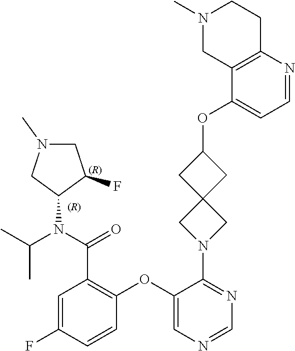

This compound was synthesized following the procedure described for the synthesis of Example 20, Step 4, starting with 1 equivalent of 5-fluoro-N-((3R,4R)-4-fluoropyrrolidin-3-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide, except that 15 equivalents of formaldehyde (37% in H₂O), 0.2 equivalent of AcOH, 3 equivalents of STAB, and 6 equivalents of Et₃N were used. After work-up, the crude product was purified by reverse phase chromatography using a SiliCycle SiliaSep C18 column (80 g) on Biotage isolera one (Mobile phase A: 10 mM NH₄HCO₃ in water, B: ACN) to obtain 5-fluoro-N-((3R,4R)-4-fluoro-1-methylpyrrolidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 35.5%; ¹H NMR (400 MHz, DMSO-d₆): δ 8.30 (s, 1H), 8.18 (d, J=5.5 Hz, 1H), 7.81-7.76 (m, 1H), 7.48-7.23 (m, 2H), 7.13-6.99 (m, 1H), 6.70-6.61 (m, 1H), 5.61-5.22 (m, 1H), 4.81-4.69 (m, 1H), 4.36-4.07 (m, 4H), 3.92-3.68 (m, 2H), 3.36 (s, 2H), 3.07-2.94 (m, 1H), 2.91-2.72 (m, 6H), 2.66-2.60 (m, 2H), 2.36 (s, 3H), 2.31-2.20 (m, 5H), 1.18-0.98 (m, 6H), one proton merged with solvent peaks; LCMS (Method A): Rt=1.66 min, m/z=634.2 [M+H]⁺; HPLC (Method A): Rt=4.83 min, 97.88%; Chiral SFC (Method N): Rt=1.25 min, 100%.

### Example 26. 5-Fluoro-N-((3R,4R)-4-fluoro-1-methylpyrrolidin-3-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 26)

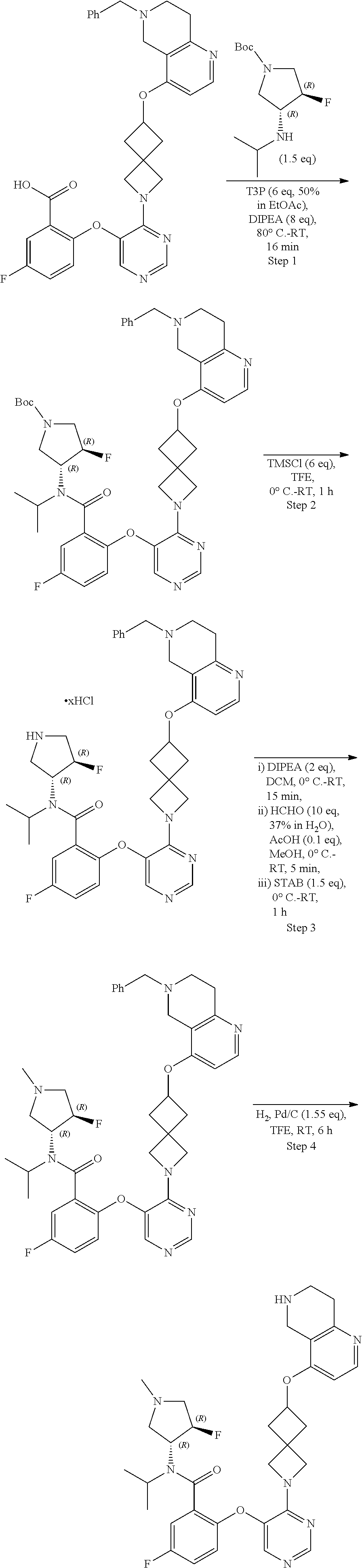

### Step 1. tert-Butyl (3R,4R)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-4-fluoropyrrolidine-1-carboxylate

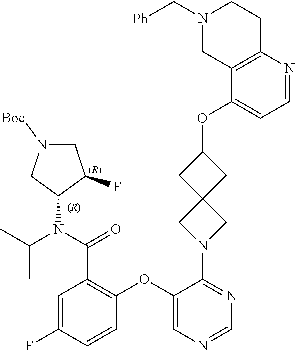

This compound was synthesized following the procedure described for the synthesis of Example 25, Step 1, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.5 equivalents of tert-butyl (3R,4R)-3-fluoro-4-(isopropylamino)pyrrolidine-1-carboxylate. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl (3R,4R)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-4-fluoropyrrolidine-1-carboxylate as a solid.

Yield: 12.6%; ¹H NMR (400 MHz, DMSO-d₆): δ 8.33-8.27 (m, 1H), 8.21-8.15 (m, 1H), 7.80-7.74 (m, 1H), 7.49-7.39 (m, 1H), 7.38-7.24 (m, 6H), 7.12-7.03 (m, 1H), 6.68-6.63 (m, 1H), 5.71-5.26 (m, 1H), 4.78-4.62 (m, 1H), 4.39-4.01 (m, 5H), 3.99-3.73 (m, 3H), 3.68 (s, 2H), 3.49-3.38 (m, 3H), 2.89-2.66 (m, 7H), 2.26-2.17 (m, 2H), 1.47-1.31 (m, 9H), 1.19-0.91 (m, 6H), two protons merged with solvent peaks; LCMS (Method A): Rt=2.32 min, m/z=796.2 [M+H]⁺, 91.21%.

### Step 2. 2-((4-(6-((6-Benzyl-5,6,7,8-tetrahydro-J, 6-naphthyridin-4-yl)oxy)-2-azaspiro[3.3]heptan-2-yl)pyrimidin-5-yl)oxy)-5-fluoro-N-((3R,4R)-4-fluoropyrrolidin-3-yl)-N-isopropylbenzamide xHCl

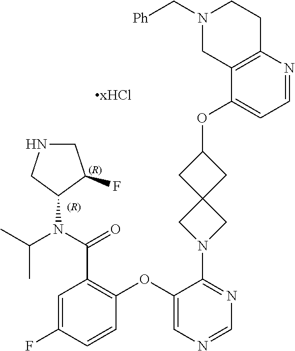

This compound was synthesized following the procedure described for the synthesis of Example 20, Step 3, starting with 1 equivalent of tert-butyl (3R,4R)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-4-fluoropyrrolidine-1-carboxylate, except that 6 equivalents of TMSCl was used. After completion, the reaction was concentrated under reduced pressure to obtain crude 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-((3R,4R)-4-fluoropyrrolidin-3-yl)-N-isopropylbenzamide xHCl as a gummy liquid. This material was used without further purification.

Yield: 80%; LCMS (Method A): Rt=1.90 min, m/z=696.3 [M+H]⁺, 84.25%.

### Step 3. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-((3R,4R)-4-fluoro-1-methylpyrrolidin-3-yl)-N-isopropylbenzamide

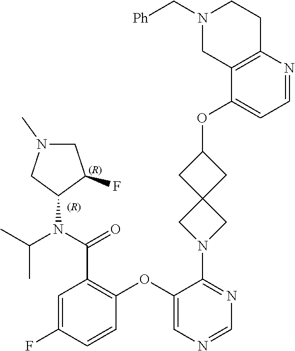

This compound was synthesized following the procedure described for the synthesis of Example 20, Step 4, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-((3R,4R)-4-fluoropyrrolidin-3-yl)-N-isopropylbenzamide hydrochloride, except that 2 equivalents of DIPEA, 0.1 equivalent of AcOH, and 1.5 equivalents of STAB were used. The reaction progress was monitored by TLC (10% MeOH in DCM). After completion, the reaction was diluted with ice-cold water, and aqueous Na₂CO₃ solution was added. The mixture was extracted with EtOAc. The combined organic layer was dried over sodium sulfate and filtered, and the filtrate was concentrated under reduced pressure to obtain crude 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-((3R,4R)-4-fluoro-1-methylpyrrolidin-3-yl)-N-isopropylbenzamide as a solid. This material was used without further purification.

Yield: 77%; LCMS (Method A). Rt=2.01 min, m/z=710.2 [M+H]⁺, 88.44%.

### Step 4: 5-Fluoro-N-((3R,4R)-4-fluoro-1-methylpyrrolidin-3-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

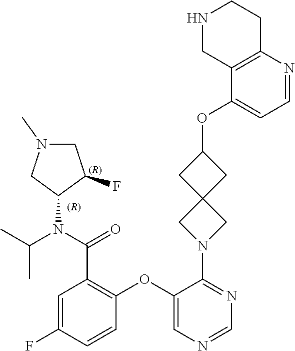

This compound was synthesized following the procedure described for the synthesis of Example 20, Step 5, starting with 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1S,3S)-3-(dimethylamino)cyclopentyl)-5-fluoro-N-isopropylbenzamide, except that 1.55 equivalents of Pd—C was used. The crude was purified by reverse phase chromatography using a SiliCycle SiliaSep C18 column (50 g) on Biotage isolera one (Mobile phase A: 10 mM NH₄HCO₃ in water, B: ACN) to obtain N-((1S,3S)-3-(dimethylamino)cyclopentyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 15.42%; ¹H NMR (400 MHz, DMSO-d₆): δ 8.30 (s, 1H), 8.15 (d, J=5.5 Hz, 1H), 7.83-7.75 (m, 1H), 7.48-7.22 (m, 2H), 7.14-6.99 (m, 1H), 6.69-6.59 (m, 1H), 5.60-5.21 (m, 1H), 4.79-4.66 (m, 1H), 4.34-4.07 (m, 4H), 3.89-3.72 (m, 2H), 3.69 (s, 2H), 3.08-2.89 (m, 4H), 2.85-2.73 (m, 3H), 2.71-2.63 (m, 3H), 2.32-2.19 (m, 5H), 1.18-0.97 (m, 6H); LCMS (Method A): Rt=1.54 min, m/z=620.2 [M+H]⁺; HPLC (Method A): Rt=4.32 min, 97.18%; Chiral SFC (Method N): Rt=1.42 min, 100%.

### Example 27. 5-Fluoro-N-((3R,4S)-4-fluoro-1-methylpyrrolidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 27)

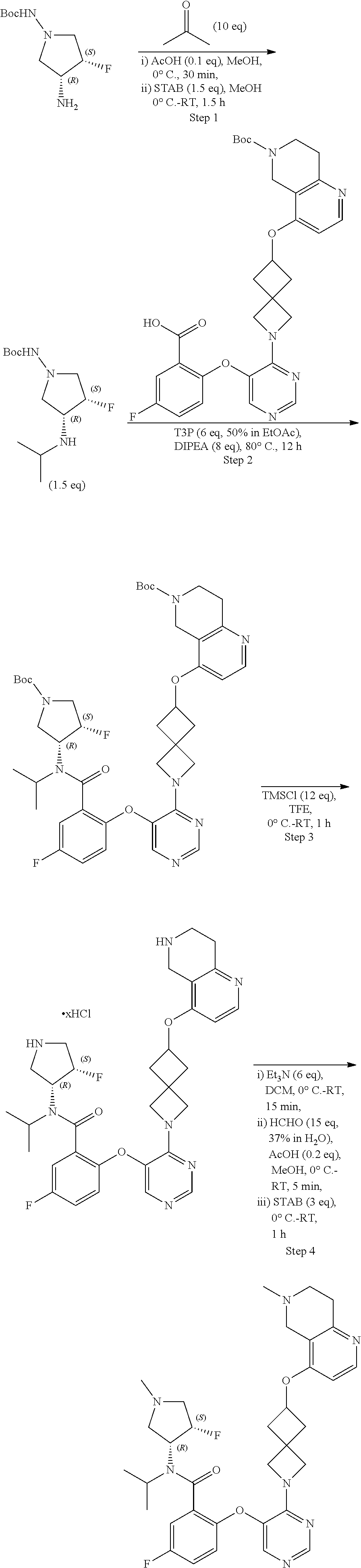

### Step 1. tert-Butyl (3S,4R)-3-fluoro-4-(isopropyl amino)pyrrolidine-1-carboxylate

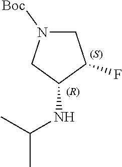

This compound was synthesized following the procedure described for the synthesis of Intermediate 20, Step 1, starting with 1 equivalent of tert-butyl (3R,4S)-3-amino-4-fluoropyrrolidine-1-carboxylate, except that 10 equivalents of acetone was used. After completion, the reaction was diluted with ice-cold water, and aqueous Na₂CO₃ solution, and the mixture was extracted with EtOAc. The combined organic layer was washed with brine solution, dried over anhydrous sodium sulfate and filtered, and the filtrate was concentrated under reduced pressure to afford crude tert-butyl (3S,4R)-3-fluoro-4-(isopropyl amino)pyrrolidine-1-carboxylate as a liquid. This material was used without further purification.

Yield: 71.6%; ¹H NMR (400 MHz, DMSO-d₆): δ 5.13-4.96 (m, 1H), 3.64-3.36 (m, 4H), 2.96-2.77 (m, 2H), 1.42-1.38 (m, 9H), 1.03-0.94 (m, 6H), one proton merged with solvent peaks.

### Step 2. tert-Butyl 4-((2-(S-(2-(((3R,4S)-1-(tert-butoxycarbonyl)-4-fluoropyrrolidin-3-yl) (isopropyl)carbamoyl)-4-fluorophenoxy)pyrimidin-4-yl)-2-azaspiro [3.3]heptan-6-yl)oxy)-7,8-dihydro-1,6-naphthyridine-6(5H)-carboxylate

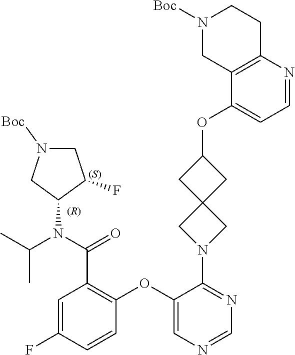

This compound was synthesized following the procedure described for the synthesis of Example 25, Step 1, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid, except that 1.5 equivalents of tert-butyl (3S,4R)-3-fluoro-4-(isopropyl amino)pyrrolidine-1-carboxylate was used. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl 4-((2-(5-(2-(((3R,4S)-1-(tert-butoxycarbonyl)-4-fluoropyrrolidin-3-yl) (isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate as a solid.

Yield: 40.5%; ¹H NMR (400 MHz, DMSO-d₆): δ 8.34-8.26 (m, 1H), 8.23 (d, J=5.5 Hz, 1H), 7.96-7.67 (m, 1H), 7.44-7.19 (m, 2H), 7.14-6.97 (m, 1H), 6.74 (br d, J=5.5 Hz, 1H), 5.31-4.85 (m, 1H), 4.83-4.70 (m, 1H), 4.37 (br s, 2H), 4.33-4.13 (m, 5H), 3.88-3.68 (m, 1H), 3.62 (br t, J=5.7 Hz, 2H), 3.53-3.30 (m, 2H), 2.88-2.74 (m, 4H), 2.31-2.20 (m, 2H), 1.55-1.48 (m, 1H), 1.45-1.00 (m, 24H), one proton merged with solvent peaks; LCMS (Method B): Rt=1.68 min, m/z=806.2 [M+H]⁺, 99.77%.

### Step 3. 5-Fluoro-N-((3R,4S)-4-fluoropyrrolidin-3-yl)-N-isopropyl-2-((4-(6-((S, 6,7,8-tetrahydro-1,6-naphthyridin-4-yl)oxy)-2-azaspiro[3.3]heptan-2-yl)pyrimidin-5-yl)oxy)benzamide

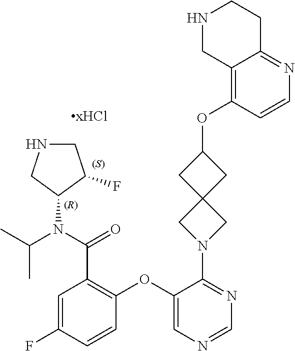

This compound was synthesized following the procedure described for the synthesis of Example 20, Step 3, starting with 1 equivalent of tert-butyl 4-((2-(5-(2-(((3R,4S)-1-(tert-butoxycarbonyl)-4-fluoropyrrolidin-3-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate, except that 12 equivalents of TMSCl was used. After completion, the reaction was concentrated under reduced pressure to afford crude 5-fluoro-N-((3R,4S)-4-fluoropyrrolidin-3-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl as a solid. This material was used without further purification.

Yield: 82%; LCMS (Method A): Rt=1.52 min, m/z=606.1 [M+H]⁺, 84.09%.

### Step 4. 5-Fluoro-N-((3R,4S)-4-fluoro-1-methylpyrrolidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

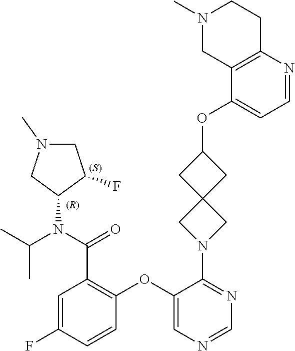

This compound was synthesized following the procedure described for the synthesis of Example 20, Step 4, starting with 1 equivalent of 5-fluoro-N-((3R,4S)-4-fluoropyrrolidin-3-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide, except that 15 equivalents of formaldehyde (37% in H₂O), 6 equivalents of Et₃N, 0.2 equivalent of AcOH, and 3 equivalents of STAB were used. After work-up, the crude was purified by reverse phase chromatography using a SiliCycle SiliaSep C18 column (24 g) on Biotage isolera one (Mobile phase A: 10 mM NH₄HCO₃ in water, B: ACN) to obtain 5-fluoro-N-((3R,4S)-4-fluoro-1-methylpyrrolidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 42.6%; ¹H NMR (400 MHz, DMSO-d₆): δ 8.33-8.27 (m, 1H), 8.18 (d, J=5.6 Hz, 1H), 7.96-7.64 (m, 1H), 7.35-7.17 (m, 1H), 7.15-6.99 (m, 2H), 6.72-6.59 (m, 1H), 4.98-4.67 (m, 2H), 4.43-4.03 (m, 5H), 3.84-3.66 (m, 1H), 3.36 (s, 2H), 2.95-2.69 (m, 7H), 2.63 (br t, J=5.8 Hz, 2H), 2.36 (s, 3H), 2.33-2.13 (m, 6H), 1.53-0.97 (m, 6H); LCMS (Method A): Rt=1.68 min, m/z=634.2 [M+H]⁺; HPLC (Method A): Rt=4.77 min, 99.62%; Chiral SFC (Method N): Rt=1.50 min, 100%.

### Example 28. 5-Fluoro-N-((3R,4S)-3-fluoro-1-methylpiperidin-4-yl)-N-isopropyl-2-((4-(6-((6-methyl-5,6,7,8-tetrahydro-1,6-naphthyridin-4-yl)oxy)-2-azaspiro 13.31 heptan-2-yl)pyrimidin-5-yl)oxy)benzamide (Compound No. 28)

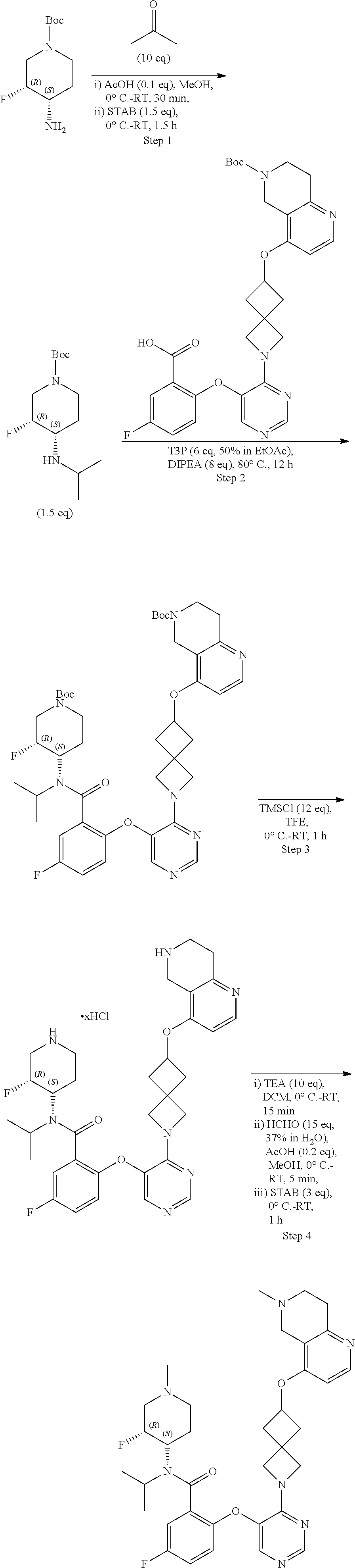

### Step 1. tert-Butyl (3R,4S)-3-fluoro-4-(isopropyl amino) piperidine-1-carboxylate Boc

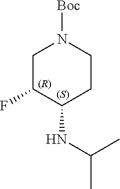

This compound was synthesized following the procedure described for the synthesis of Example 20, Step 1, starting with 1 equivalent of tert-butyl (3R,4S)-4-amino-3-fluoropiperidine-1-carboxylate (200 mg, 0.916 mmol), except that 10 equivalents of acetone was used. After completion, the reaction was diluted with ice-cold water and aqueous Na₂CO₃ solution, and the mixture was extracted with EtOAc. The combined organic layer was washed with brine solution, dried over sodium sulfate and filtered, and the filtrate was concentrated under reduced pressure to obtain crude tert-butyl (3R,4S)-3-fluoro-4-(isopropylamino)piperidine-1-carboxylate as a solid. This material was sued without further purification.

Yield: 84%; ¹H NMR (400 MHz, DMSO-d₆): δ 4.86-4.55 (m, 1H), 4.34-3.81 (m, 2H), 2.97-2.89 (m, 1H), 2.81-2.65 (m, 2H), 1.66-1.57 (m, 1H), 1.39 (s, 9H), 1.35-1.23 (m, 1H), 1.00-0.92 (m, 6H), two protons merged with solvent peaks; LCMS (Method B): Rt=1.14 min, m/z=261.3 [M+H]⁺, 99.71%.

### Step 2. tert-Butyl 4-((2-(5-(2-(((3R,4S)-1-(tert-butoxycarbonyl)-3-fluoropiperidin-4-yl) (isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate

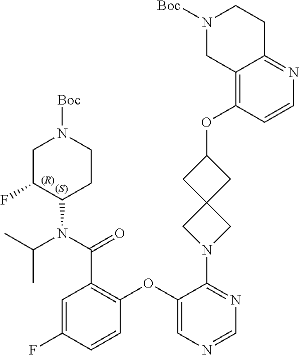

This compound was synthesized following the procedure described for the synthesis of Example 25, Step 1, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic, except that 1.5 equivalents of tert-butyl (3R,4S)-3-fluoro-4-(isopropyl amino)piperidine-1-carboxylate was used. After work-up, the crude was purified by reverse phase chromatography using a SiliCycle SiliaSep C18 column (80 g) on Biotage isolera one (Mobile phase A: 10 mM NH₄HCO₃ in water, B: ACN) to afford tert-butyl 4-((2-(5-(2-(((3R,4S)-1-(tert-butoxycarbonyl)-3-fluoropiperidin-4-yl) (isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate as a solid.

Yield: 52.6%; ¹H NMR (400 MHz, DMSO-d₆): δ 8.34-8.26 (m, 1H), 8.23 (d, J=5.7 Hz, 11H), 7.87-7.74 (m, 1H), 7.38-7.21 (m, 2H), 7.17-7.00 (m, 11H), 6.74 (br d, J=5.7 Hz, 1H), 5.01-4.70 (m, 2H), 4.38 (s, 2H), 4.15 (br d, J=4.9 Hz, 4H), 4.07-3.80 (m, 2H), 3.73-3.44 (m, 4H), 3.03-2.71 (m, 6H), 2.35-2.20 (m, 2H), 2.07-1.92 (m, 1H), 1.51-1.00 (m, 24H), one proton merged with solvent peaks; LCMS (Method A): Rt=2.30 min, m/z=820.3 [M+H]⁺, 99.62%.

### Step 3. 5-Fluoro-N-((3R,4S)-3-fluoropiperidin-4-yl)-N-isopropyl-2-((4-(6-((S, 6,7,8-tetrahydro-1,6-naphthyridin-4-yl)oxy)-2-azaspiro[3.3]heptan-2-yl)pyrimidin-5-yl)oxy)benzamide xHCl

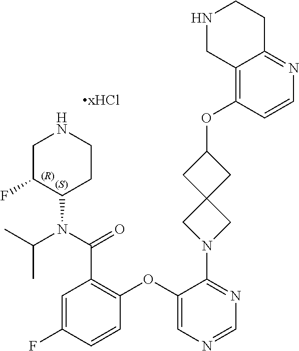

This compound was synthesized following the procedure described for the synthesis of Example 20, Step 3, starting with tert-butyl 4-((2-(5-(2-(((3R,4S)-1-(tert-butoxycarbonyl)-3-fluoropiperidin-4-yl) (isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate, except that 12 equivalents of TMSCl was used. After completion, the reaction was concentrated under reduced pressure to obtain crude 5-fluoro-N-((3R,4S)-3-fluoropiperidin-4-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl as a solid. This material was used without further purification.

Yield: 82%; LCMS (Method A): Rt=1.45 min, m/z=620.2 [M+H]⁺, 95.21%.

### Step 4. 5-Fluoro-N-((3R,4S)-3-fluoro-1-methylpiperidin-4-yl)-N-isopropyl-2-((4-(6-((6-methyl-S, 6,7,8-tetrahydro-1,6-naphthyridin-4-yl)oxy)-2-azaspiro[3.3]heptan-2-yl)pyrimidin-5-yl)oxy)benzamide

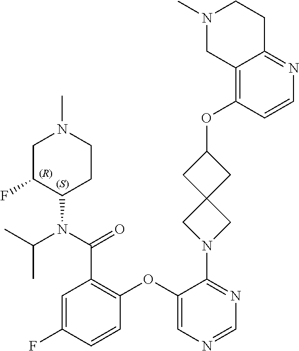

This compound was synthesized following the procedure described for the synthesis of Example 20, Step 4, starting with 1 equivalent of 5-fluoro-N-((3R,4S)-3-fluoropiperidin-4-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide dihydrochloride, except that 15 equivalents of formaldehyde (37% in H₂O), 10 equivalents of TEA, 0.2 equivalent of AcOH, and 3 equivalents of STAB were used. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain 5-fluoro-N-((3R,4S)-3-fluoro-1-methylpiperidin-4-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 37%; ¹H NMR (400 MHz, DMSO-d₆): δ 8.32-8.25 (m, 1H), 8.19 (d, J=5.7 Hz, 1H), 7.80-7.59 (m, 1H), 7.40-7.23 (m, 2H), 7.16-7.03 (m, 1H), 6.66 (d, J=5.7 Hz, 1H), 5.82-5.78 (m, 1H), 4.93-4.70 (m, 2H), 4.38-4.08 (m, 4H), 3.85-3.61 (m, 1H), 3.37 (s, 2H), 3.28-3.16 (m, 11H), 2.94-2.74 (m, 6H), 2.63 (br t, J=5.8 Hz, 3H), 2.37 (s, 3H), 2.31-2.21 (m, 2H), 2.11-2.05 (m, 3H), 1.95-1.64 (m, 2H), 1.55-1.02 (m, 6H); LCMS (Method A): Rt=1.61 min, m/z=648.2 [M+H]⁺; HPLC (Method A): Rt=4.69 min, 99.80%; Chiral SFC (Method Z): Rt=4.35 min, 100%.

### Example 29. 5-Fluoro-N-((3S,4S)-3-fluoro-1-methylpiperidin-4-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 29)

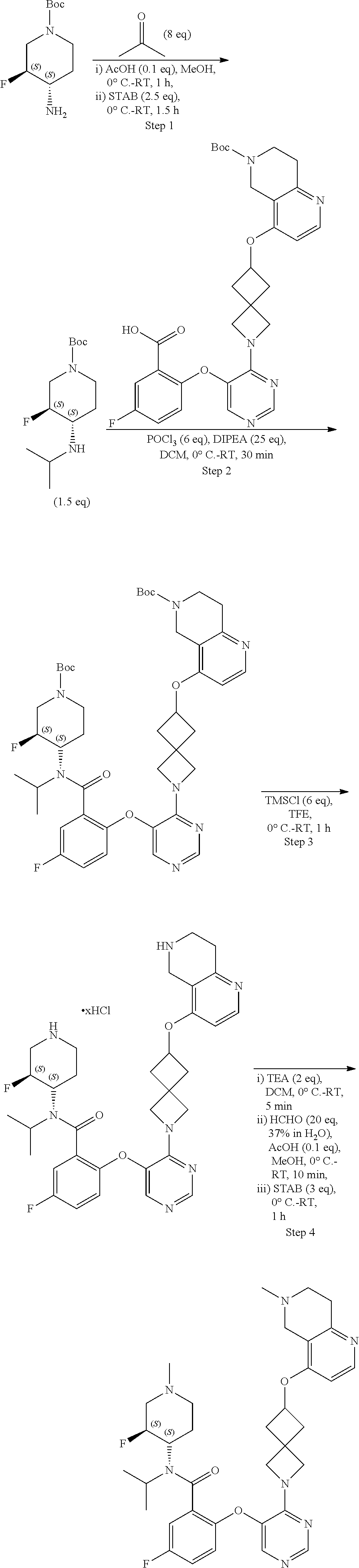

### Step 1. tert-Butyl (3S,4S)-3-fluoro-4-(isopropylamino)piperidine-1-carboxylate

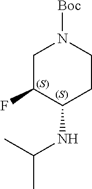

This compound was synthesized following the procedure described for the synthesis of Example 20, Step 1, starting with 1 equivalent of tert-butyl (3S,4S)-4-amino-3-fluoropiperidine-1-carboxylate, except that 2.5 equivalents of STAB were used. After completion, the reaction was concentrated under reduced pressure, the residue was diluted with water, and the mixture was extracted with EtOAc. The combined organic layer was dried over anhydrous sodium sulfate and filtered, and the filtrate was concentrated under reduced pressure to afford crude tert-butyl (3S,4S)-3-fluoro-4-(isopropylamino)piperidine-1-carboxylate as a solid. This material was used without further purification.

Yield: 89%; ¹H NMR (400 MHz, DMSO-d₆): δ 4.36-4.17 (m, 1H), 3.81-3.65 (m, 1H), 3.58-3.41 (m, 1H), 3.30-3.22 (m, 1H), 3.19-3.07 (m, 1H), 2.95-2.74 (m, 2H), 1.87-1.74 (m, 1H), 1.64-1.53 (m, 1H), 1.39 (s, 9H), 1.31-1.21 (m, 1H), 1.03-0.93 (m, 6H); LCMS (Method A): Rt=1.84 min, m/z=261.1 [M+H]⁺, 90.26%.

### Step 2. tert-Butyl 4-((2-(S-(2-(((3S,4S)-1-(tert-butoxycarbonyl)-3-fluoropiperidin-4-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate

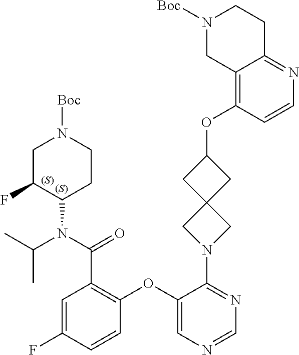

This compound was synthesized following the procedure described for the synthesis of Example 20, Step 2, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.5 equivalents of tert-butyl (3S,4S)-3-fluoro-4-(isopropylamino)piperidine-1-carboxylate, except that 25 equivalents of DIPEA were used. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain tert-butyl 4-((2-(5-(2-(((3S,4S)-1-(tert-butoxycarbonyl)-3-fluoropiperidin-4-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate as a solid.

Yield: 43.5%; LCMS (Method A): Rt=2.31 min, m/z=820.3 [M+H]⁺; HPLC (Method A): Rt=7.20 min, 82.93%.

### Step 3. 5-Fluoro-N-((3S,4S)-3-fluoropiperidin-4-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl

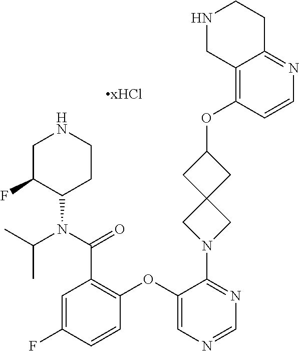

This compound was synthesized following the procedure described for the synthesis of Example 20, Step 3, starting with 1 equivalent of tert-butyl 4-((2-(5-(2-(((3S,4S)-1-(tert-butoxycarbonyl)-3-fluoropiperidin-4-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate, except that 6 equivalents of TMSCl was used. After completion, the reaction was concentrated under reduced pressure to obtain crude 5-fluoro-N-((3S,4S)-3-fluoropiperidin-4-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl as a solid. This material was used without further purification.

Yield: 86%; LCMS (Method A): Rt=1.52 min, m/z=620.2 [M+H]⁺, 83.60%.

### Step 4. 5-Fluoro-N-((3S,4S)-3-fluoro-1-methylpiperidin-4-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

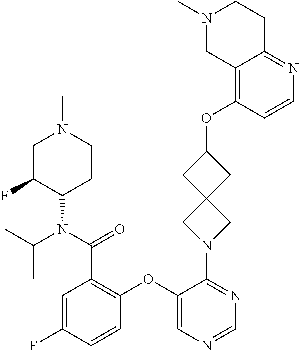

This compound was synthesized following the procedure described for the synthesis of Example 20, Step 4, starting with 1 equivalent of 5-fluoro-N-((3S,4S)-3-fluoropiperidin-4-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide dihydrochloride, except that 20 equivalents of formaldehyde, 0.1 equivalent of AcOH, and 3 equivalents of STAB were used. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain 5-fluoro-N-((3S,4S)-3-fluoro-1-methylpiperidin-4-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 39.5%; ¹H NMR (400 MHz, DMSO-d₆): δ 8.31-8.25 (m, 1H), 8.18 (d, J=5.5 Hz, 1H), 7.81-7.71 (m, 1H), 7.35-6.94 (m, 3H), 6.66 (d, 1=5.6 Hz, 1H), 5.67-5.37 (m, 1H), 4.74 (quin, J=6.6 Hz, 1H), 4.35-4.07 (m, 4H), 3.85-3.57 (m, 1H), 3.37 (br s, 2H), 3.23-3.02 (m, 2H), 2.87-2.69 (m, 5H), 2.69-2.58 (m, 3H), 2.37 (s, 3H), 2.30-2.13 (m, 5H), 2.07-1.55 (m, 3H), 1.51-0.92 (m, 6H); LCMS (Method A): Rt=1.70 min, m/z=648.2 [M+H]⁺, HPLC (Method A): Rt=5.07 min, 98.94%; Chiral SFC (Method AA): Rt=1.33 min, 100%.

### Example 30. 5-Fluoro-N-((3S,4R)-3-fluoro-1-methylpiperidin-4-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 30)

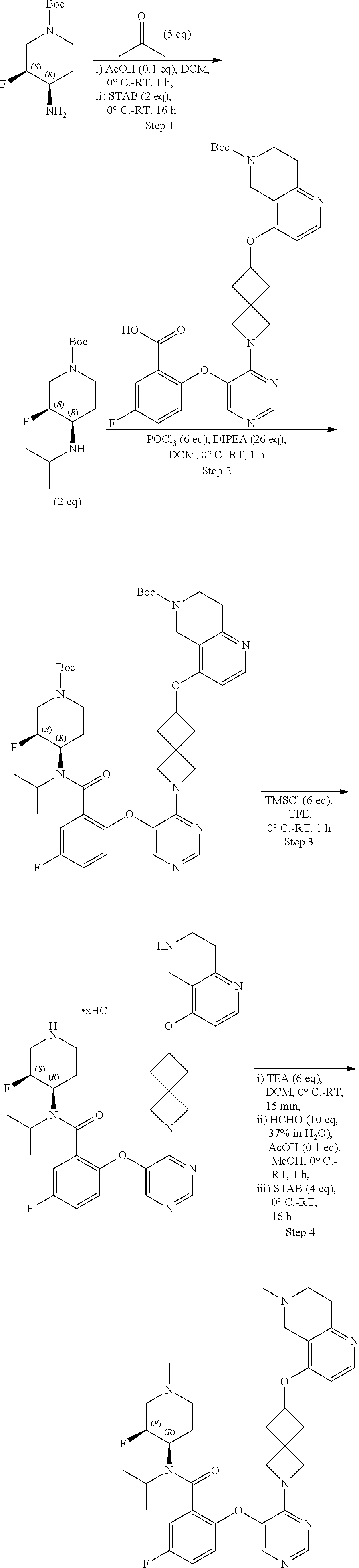

### Step 1. tert-Butyl (3S,4R)-3 fluoro-4-(isopropylamino)piperidine-1-carboxylate

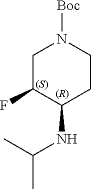

This compound was synthesized following the procedure described for the synthesis of Example 20, Step 1, starting with 1 equivalent of tert-butyl (3S,4R)-4-amino-3-fluoropiperidine-1-carboxylate, except that 5 equivalents of acetone and 2 equivalents of STAB were used, and DCM was used as the solvent. After completion, the reaction was quenched with water and the mixture was extracted with DCM. The combined organic layer was dried over sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN). The fractions were concentrated under reduced pressure to afford tert-butyl (3S,4R)-3-fluoro-4-(isopropylamino)piperidine-1-carboxylate as a solid.

Yield; 47.4%; LCMS (Method A): Rt=1.68 min, m/z=261.1 [M+H]⁺, 83.20%.

### Step 2. tert-Butyl 4-((2-(5-(2-(((3S,4R)-1-(tert-butoxycarbonyl)-3-fluoropiperidin-4-yl)(isopropyl)carbamyl)-4-fluorophenoxy)pyrimidin-4-yl)-2-azaspiro[3.3]heptan-6-yl)oxy)-7,8-dihydro-1,6-naphthyridine-6(5H)-carboxylate

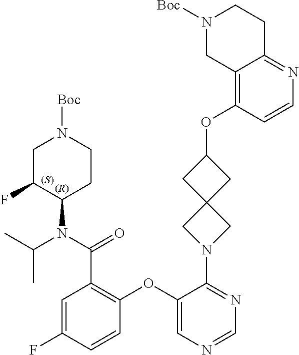

This compound was synthesized following the procedure described for the synthesis of Example 20, Step 2, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid and 2 equivalents of tert-butyl (3S,4R)-3-fluoro-4-(isopropylamino)piperidine-1-carboxylate. After work-up, the crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure to afford tert-butyl 4-((2-(5-(2-(((3S,4R)-1-(tert-butoxycarbonyl)-3-fluoropiperidin-4-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate as a solid.

Yield: 42.2%; LCMS (Method A): Rt=2.28 min, m/z=820.4 [M+H]⁺, 71.87%.

### Step 3. 5-Fluoro-N-((3S,4R)-3-fluoropiperidin-4-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl

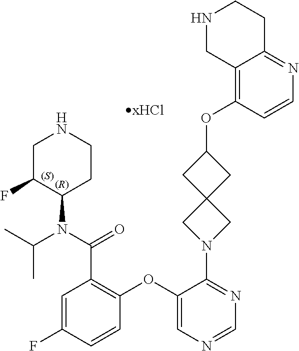

This compound was synthesized following the procedure described for the synthesis of Example 20, Step 3, starting with 1 equivalent of tert-butyl 4-((2-(5-(2-(((3S,4R)-1-(tert-butoxycarbonyl)-3-fluoropiperidin-4-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate, except that 6 equivalents of TMSCl was used. After completion, the reaction was concentrated under reduced pressure to afford crude 5-fluoro-N-((3S,4R)-3-fluoropiperidin-4-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl as a sticky solid. This material was used without further purification.

Yield: 73.4%; LCMS (Method A): Rt=1.46 min, m/z=620.4 [M+H]⁺, 54.83%.

### Step 4. 5-Fluoro-N-((3,5,4R)-3-fluoro-1-methylpiperidin-4-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

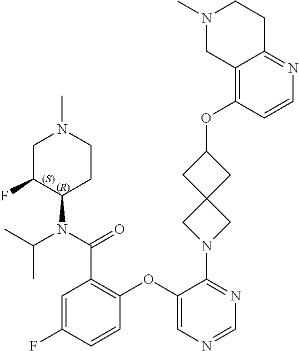

This compound was synthesized following the procedure described for the synthesis of Example 20, Step 4, starting with 1 equivalent of 5-fluoro-N-((3S,4R)-3-fluoropiperidin-4-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl, except that 6 equivalents of TEA, 0.1 equivalent of AcOH and 4 equivalents of STAB were used. After work-up, the crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford 5-fluoro-N-((3S,4R)-3-fluoro-1-methylpiperidin-4-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 21.32%; ¹H NMR (400 MHz, DMSO-d₆): δ 8.31-8.26 (m, 1H), 8.19 (d, J=5.6 Hz, 1H), 7.74-7.67 (m, 1H), 7.37 (dd, J=3.1, 8.3 Hz, 1H), 7.32-7.23 (m, 1H), 7.14-7.04 (m, 1H), 6.66 (d, J=5.8 Hz, 1H), 4.89-4.70 (m, 2H), 4.37-4.09 (m, 5H), 3.79-3.62 (m, 1H), 3.37 (s, 2H), 2.94-2.69 (m, 6H), 2.63 (br t, J=6.0 Hz, 2H), 2.37 (s, 3H), 2.29-2.21 (m, 2H), 2.18-2.05 (m, 5H), 1.94-1.76 (m, 2H), 1.52-1.02 (m, 6H); LCMS (Method A): Rt=1.64 min, m/z=648.2 [M+H]%; HPLC (Method A): Rt=4.68 min, 97.25%; Chiral SFC (Method C): Rt=2.16 min, 94.57%.

### Example 31. 5-Fluoro-N-((3R,4R)-3-fluoro-1-methylpiperidin-4-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 31)

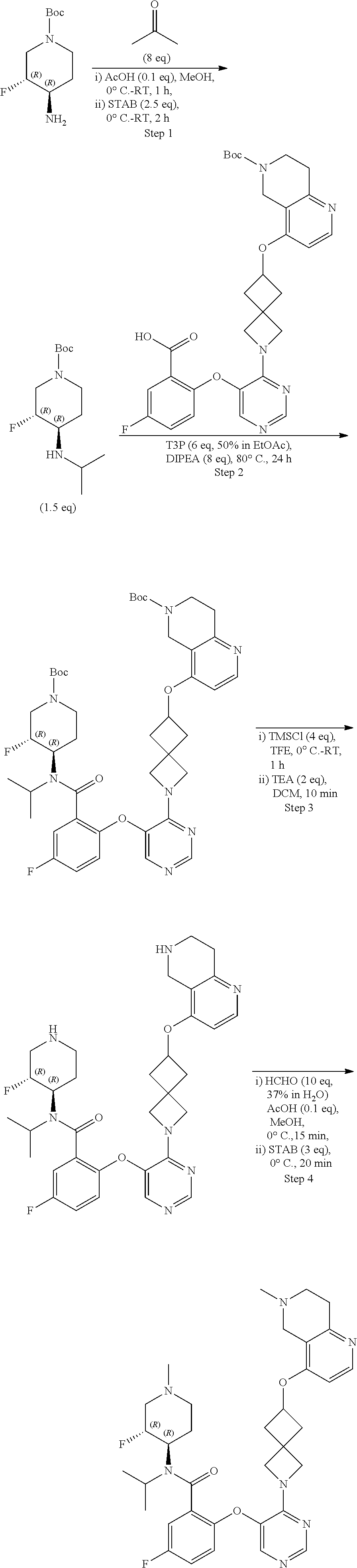

### Step 1. tert-Butyl (3R,4R)-3-fluoro-4-(isopropylamino)piperidine-1-carboxylate

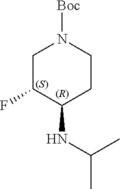

This compound was synthesized following the procedure described for the synthesis of Example 20, Step 1, starting with 1 equivalent of tert-butyl (3R,4R)-4-amino-3-fluoropiperidine-1-carboxylate, except that 2.5 equivalents of STAB were used. After completion, saturated sodium bicarbonate solution was added to the reaction mixture and the resulting mixture was extracted with DCM. The combined organic layer was dried over anhydrous sodium sulfate and filtered, and the filtrate was concentrated under reduced pressure to afford crude tert-butyl (3R,4R)-3-fluoro-4-(isopropylamino)piperidine-1-carboxylate as a liquid. This crude was used without further purification.

Yield: 67.7%; ¹H NMR (400 MHz, DMSO-d₆): δ 4.37-4.17 (m, 1H), 3.79-3.66 (m, 1H), 3.53-3.41 (m, 1H), 3.19-3.10 (m, 1H), 2.91-2.79 (m, 2H), 1.85-1.75 (m, 1H), 1.40 (s, 9H), 1.31-1.19 (m, 1H), 0.99 (d, J=6.3 Hz, 3H), 0.96 (d, J=6.1 Hz, 3H), two protons merged with solvent peaks; LCMS (Method A): Rt=1.70 min, m/z=261.1 [M+H]⁺, 80.73%.

### Step 2. tert-Butyl 4-((2-(S-(2-(((3R,4R)-1-(tert-butoxycarbonyl)-3-fluoropiperidin-4-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate

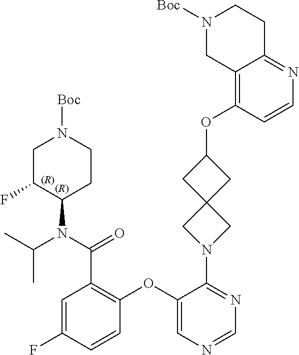

This compound was synthesized following the procedure described for the synthesis of Example 25, Step 1, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid, except that 1.5 equivalents of tert-butyl (3R,4R)-3-fluoro-4-(isopropylamino)piperidine-1-carboxylate was used. After completion, the reaction was quenched with aqueous NaHCO₃ solution, and the mixture was extracted with EtOAc. The combined organic layer was dried over anhydrous sodium sulfate and filtered, and the filtrate was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN) to obtain tert-butyl 4-((2-(5-(2-(((3R,4R)-1-(tert-butoxycarbonyl)-3-fluoropiperidin-4-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate as a solid.

Yield: 32.1%; ¹H NMR (400 MHz, DMSO-d₆): δ 8.31-8.25 (m, 1H), 8.23 (br d, J=5.4 Hz, 1H), 7.80-7.68 (m, 1H), 7.36-7.04 (m, 3H), 6.78-6.71 (m, 1H), 5.50-5.25 (m, 1H), 4.83-4.73 (m, 1H), 4.41-4.33 (m, 2H), 4.32-4.10 (m, 5H), 4.00-3.72 (m, 2H), 3.61 (br t, J=5.6 Hz, 2H), 3.54-3.44 (m, 1H), 3.00-2.74 (m, 7H), 2.31-2.21 (m, 2H), 1.47-1.32 (m, 18H), 1.19-0.90 (m, 6H); LCMS (Method A): Rt=2.31 min, m/z=820.2 [M+H]⁺, 97.60.

### Step 3. 5-Fluoro-N-((3R,4R)-3-fluoropiperidin-4-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

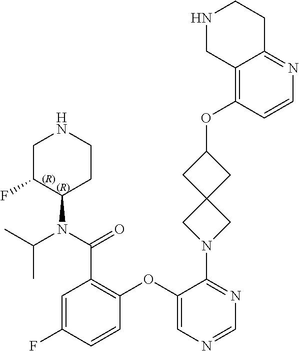

This compound was synthesized following the procedure described for the synthesis of Example 20, Step 3, starting with 1 equivalent of tert-butyl 4-((2-(5-(2-(((3R,4R)-1-(tert-butoxycarbonyl)-3-fluoropiperidin-4-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate. After completion, the reaction was concentrated under reduced pressure to obtain 5-Fluoro-N-((3R,4R)-3-fluoropiperidin-4-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl. The crude was dissolved in DCM, TEA was added and the mixture was stirred for 10 min at RT. The mixture was concentrated under reduced pressure to obtain crude 5-fluoro-N-((3R,4R)-3-fluoropiperidin-4-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a sticky solid. This material was used without further purification.

Quantitative yield; LCMS (Method A): Rt=1.49 min, m/z=620.1 [M+H]⁺, 95.98%.

### Step 4. 5-Fluoro-N-((3R,4R)-3-fluoro-1-methylpiperidin-4-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

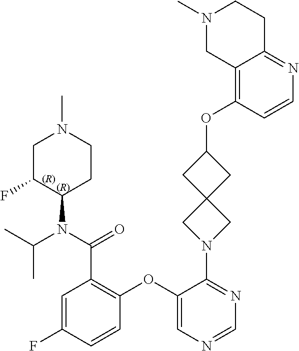

To a stirred solution of 5-fluoro-N-((3R,4R)-3-fluoropiperidin-4-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (180 mg, 0.290 mmol) in MeOH (2 mL), formaldehyde (0.216 mL, 2.90 mmol, 37% in H₂O) and AcOH (1.663 p L, 0.029 mmol) were added at 0° C. The reaction was stirred at the RT for 15 min. To this reaction mixture, STAB (185 mg, 0.871 mmol) was added at 0° C. and the reaction was stirred at 0° C. for 20 min. The reaction progress was monitored by TLC (10% MeOH in DCM). After completion, the reaction was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN) to obtain 5-fluoro-N-((3R,4R)-3-fluoro-1-methylpiperidin-4-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (45 mg, 23.59% yield) as a solid. ¹H NMR (400 MHz, DMSO-d₆): δ 8.33-8.25 (m, 1H), 8.18 (d, J=5.6 Hz, 1H), 7.80-7.70 (m, 1H), 7.35-6.92 (m, 3H), 6.66 (d, J=5.6 Hz, 1H), 5.67-5.38 (m, 1H), 4.74 (quin, J=6.5 Hz, 1H), 4.34-4.06 (m, 4H), 3.85-3.73 (m, 1H), 3.71-3.57 (m, 1H), 3.37 (br s, 2H), 3.23-3.05 (m, 2H), 2.86-2.72 (m, 4H), 2.70-2.60 (m, 3H), 2.37 (s, 3H), 2.30-2.13 (m, 5H), 2.07-1.54 (m, 2H), 1.51-0.91 (m, 6H), one proton merged with solvent peaks; LCMS (Method A): Rt=1.71 min, m/z=648.2 [M+H]⁺; HPLC (Method A): Rt=5.08 min, 98.63%; Chiral SFC (Method C): Rt=2.07 min, 100%.

### Example 32. 5-Fluoro-N-((3S,5R)-5-fluoro-1-methylpiperidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 32)

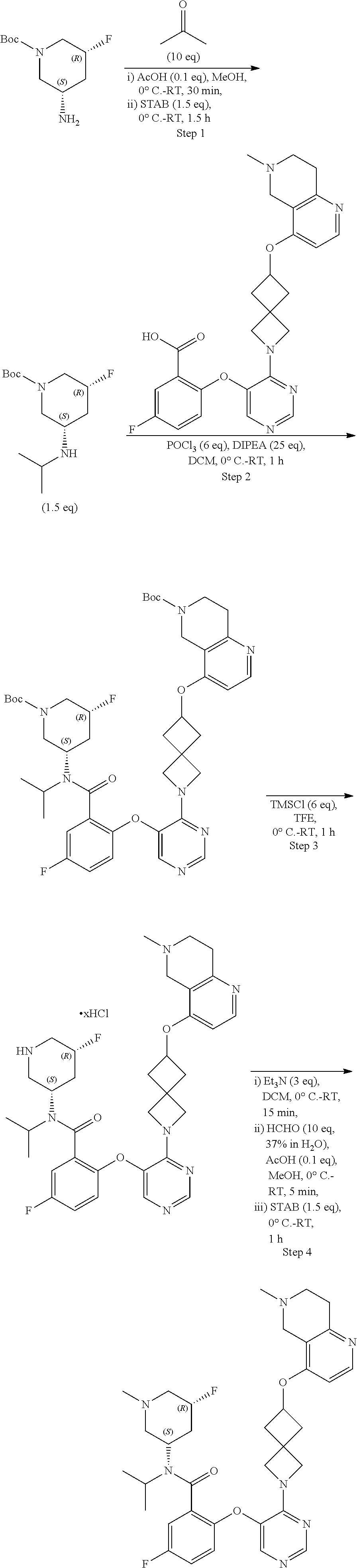

### Step 1. tert-Butyl (3R,5S)-3-fluoro-5-(isopropylamino)piperidine-1-carboxylate

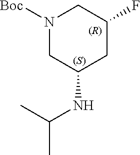

This compound was synthesized following the procedure described for the synthesis of Example 20, Step 1, starting with 1 equivalent of tert-butyl (3S,5R)-3-amino-5-fluoropiperidine-1-carboxylate, except that 10 equivalents of acetone was used. After completion, the reaction was diluted with ice-cold water and aqueous Na₂CO₃ solution, and the mixture was extracted with EtOAc. The combined organic layer was washed with brine solution, dried over anhydrous sodium sulfate and filtered, and the filtrate was concentrated under reduced pressure to afford crude tert-butyl (3R,5S)-3-fluoro-5-(isopropylamino)piperidine-1-carboxylate as a liquid. This material was used without further purification.

Yield: 77%; LCMS (Method A): Rt=1.70 min, m/z=261.1 [M+H]⁺, 91.65%.

### Step 2. tert-Butyl (3R,5S)-3-fluoro-5-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)piperidine-1-carboxylate

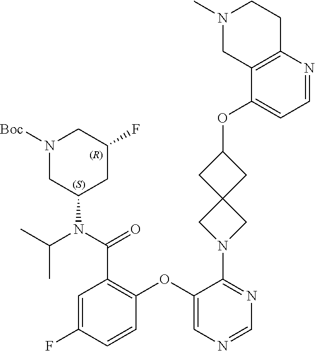

This compound was synthesized following the procedure described for the synthesis of Example 20, Step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.5 equivalents of tert-butyl (3R,5S)-3-fluoro-5-(isopropylamino)piperidine-1-carboxylate, except that 25 equivalents of DIPEA was used. After completion, the reaction was diluted with ice-cold water, and aqueous Na₂CO₃ solution, and the mixture was extracted with EtOAc. The combined organic layer was washed with brine solution, dried over anhydrous sodium sulfate, and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain tert-butyl (3R,5S)-3-fluoro-5-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)piperidine-1-carboxylate as a solid.

Yield: 17.24%; LCMS (Method A): Rt=2.04 min, m/z=734.4 [M+H]⁺, 89.00%.

### Step 3. 5-Fluoro-N-((3S,5R)-5-fluoropiperidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl

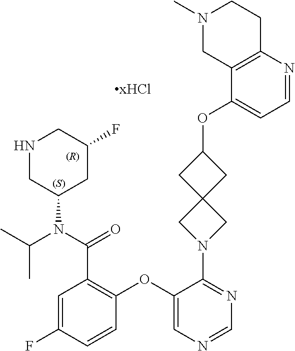

This compound was synthesized following the procedure described for the synthesis of Example 20, Step 3, starting with 1 equivalent of tert-butyl (3R,5S)-3-fluoro-5-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)piperidine-1-carboxylate, except that 6 equivalents of TMSCl was used. After completion, the reaction was concentrated under reduced pressure to obtain crude 5-fluoro-N-((3S,5R)-5-fluoropiperidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl as a solid. This material was used without further purification.

Yield: 97%; LCMS (Method B): Rt=0.91 min, m/z=634.3 [M+H]⁺, 98.68%.

### Step 4. 5-Fluoro-N-((3S,5R)-5-fluoro-1-methylpiperidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

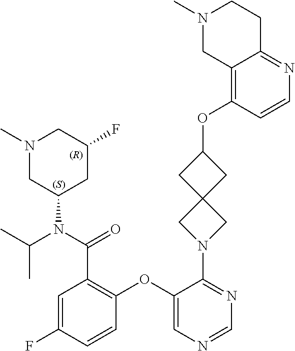

This compound was synthesized following the procedure described for the synthesis of Example 20, Step 4, starting with 1 equivalent of 5-fluoro-N-((3S,5R)-5-fluoropiperidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide hydrochloride, except that 0.1 equivalent of AcOH, 1.5 equivalents of STAB, and 3 equivalents of Et₃N were used. After work-up, the crude was purified by reverse phase chromatography using a SiliCycle SiliaSep C18 column (40 g) on Biotage isolera one (Mobile phase A: 10 mM NH₄HCO₃ in water, B: ACN) to obtain 5-fluoro-N-((3S,5R)-5-fluoro-1-methylpiperidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 32.0%; ¹H NMR (400 MHz, DMSO-d₆): δ 8.33-8.27 (m, 1H), 8.18 (d, J=5.6 Hz, 1H), 7.82-7.73 (m, 1H), 7.36-7.21 (m, 2H), 7.14-7.01 (m, 1H), 6.69-6.62 (m, 1H), 4.82-4.72 (m, 1H), 4.35-4.10 (m, 4H), 3.82-3.60 (m, 1H), 3.42-3.36 (m, 2H), 3.29-3.18 (m, 1H), 3.10-3.01 (m, 1H), 2.88-2.74 (m, 5H), 2.66-2.58 (m, 3H), 2.37 (s, 3H), 2.31-2.17 (m, 5H), 2.12-2.01 (m, 1H), 1.92-1.84 (m, 1H), 1.48-0.94 (m, 6H), two protons merged in solvent peaks; LCMS (Method A): Rt=1.71 min, m/z=648.1 [M+H]⁺; HPLC (Method A): Rt=5.04 min, 99.29%; Chiral SFC (Method Q): Rt=1.80 min, 100%.

### Example 33. 5-Fluoro-N-((3S,5S)-5-fluoro-1-methylpiperidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 33)

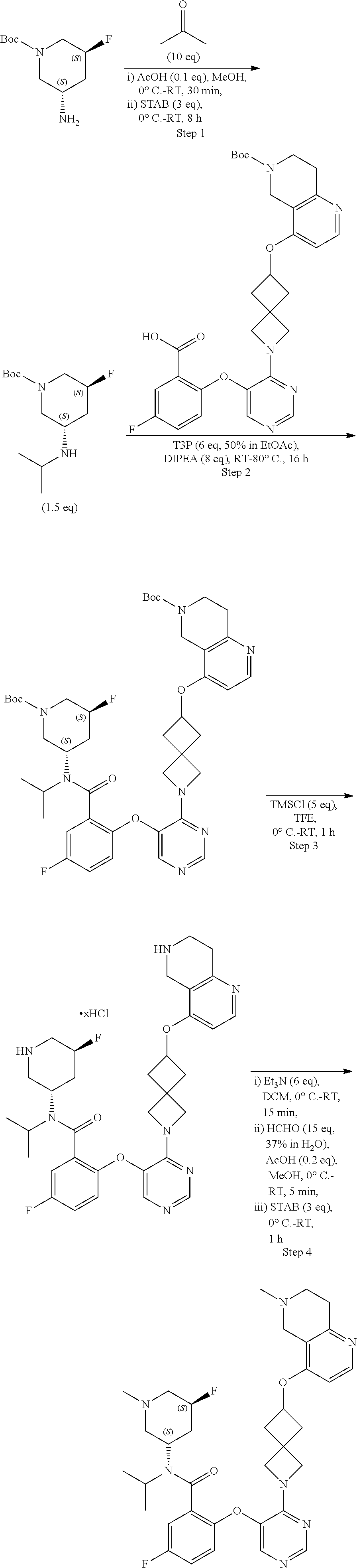

### Step 1. tert-Butyl (3,5S)-3-fluoro-5-(isopropylamino)piperidine-1-carboxylate

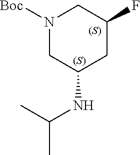

This compound was synthesized following the procedure described for the synthesis of Example 20, Step 1, starting with 1 equivalent of tert-butyl (3S,5S)-3-amino-5-fluoropiperidine-1-carboxylate, except that 10 equivalents of acetone and 3 equivalents of STAB were used. After completion, saturated sodium bicarbonate solution was added and the mixture was extracted with DCM. The combined organic layer was concentrated under reduced pressure to obtain crude tert-butyl (3S,5S)-3-fluoro-5-(isopropylamino)piperidine-1-carboxylate as a liquid. This material was used without further purification.

Yield: 88%; LCMS-ELSD (Method A): Rt=1.75 min, m/z=261.1 [M+H]⁺, 93.13%.

### Step 2. tert-Butyl 4-((2-(5-(2-(((3S,5S)-1-(tert-butoxycarbonyl)-5-fluoropiperidin-3-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate

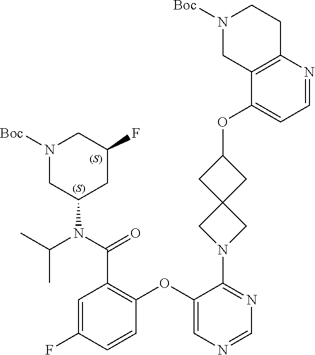

This compound was synthesized following the procedure described for the synthesis of Example 25, Step 1, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid, except that 1.5 equivalents of tert-butyl (3S,5S)-3-fluoro-5-(isopropylamino)piperidine-1-carboxylate was used. After completion, the reaction was quenched with water and the mixture was extracted with EtOAc. The combined organic layer was washed with brine solution, dried over sodium sulfate, and filtered. The filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain tert-butyl 4-((2-(5-(2-(((3S,5S)-1-(tert-butoxycarbonyl)-5-fluoropiperidin-3-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate as a solid.

Yield: 51.0%; LCMS (Method A): Rt=2.27 min, m/z=820.1 [M+H]⁺, 96.55%.

### Step 3. 5-Fluoro-N-((3S,5S)-5-fluoropiperidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl

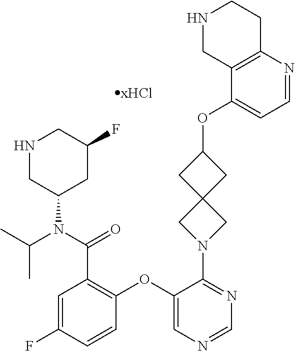 [SMILES: CC(C)N(C(=O)c1cc(F)ccc1Oc1cncnc1N1CC2(CC(Oc3ccnc4c3CNCC4)C2)C1)[C@@H]1CNC[C@@H](F)C1]

This compound was synthesized following the procedure described for the synthesis of Example 20, Step 3, starting with 1 equivalent of tert-butyl 4-((2-(5-(2-(((3S,5S)-1-(tert-butoxycarbonyl)-5-fluoropiperidin-3-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate, except that 5 equivalents of TMSCl was used. After completion, the reaction was concentrated under reduced pressure to obtain crude 5-fluoro-N-((3S,5S)-5-fluoropiperidin-3-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl as a solid. This material was used without further purification. Quantitative yield; LCMS (Method A): Rt=1.47 min, m/z=620.1 [M+H]⁺, 97.18%.

### Step 4. 5-Fluoro-N-((3S,5S)-5-fluoro-1-methylpiperidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

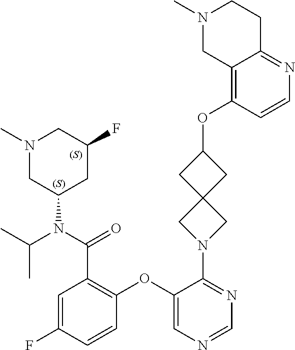

This compound was synthesized following the procedure described for the synthesis of Example 20, Step 4, starting with 1 equivalent of 5-fluoro-N-((3S,5S)-5-fluoropiperidin-3-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide dihydrochloride, except that 15 equivalents of formaldehyde (37% in H₂O), 0.2 equivalent of AcOH, 3 equivalents of STAB, and 6 equivalents of TEA were used. After work-up, the crude was purified by reverse phase chromatography using a SiliCycle SiliaSep C18 column (40 g) on Biotage isolera one (Mobile phase A: 10 mM NH₄HCO₃ in water, B: ACN) to obtain 5-fluoro-N-((3S,5S)-5-fluoro-1-methylpiperidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 60.5%; ¹H NMR (400 MHz, DMSO-d₆): δ 8.36-8.28 (m, 1H), 8.18 (d, J=5.5 Hz, 1H), 7.82-7.62 (m, 1H), 7.36-7.17 (m, 2H), 7.11-6.90 (m, 1H), 6.71-6.61 (m, 1H), 5.05-4.81 (m, 1H), 4.80-4.67 (m, 1H), 4.36-4.05 (m, 4H), 3.84-3.68 (m, 1H), 3.63-3.47 (m, 1H), 3.36 (br s, 2H), 2.98-2.73 (m, 6H), 2.63 (br t, J=5.8 Hz, 2H), 2.37 (s, 3H), 2.30-2.19 (m, 3H), 2.18-2.03 (m, 3H), 1.76 (s, 2H), 1.49-0.97 (m, 6H), one proton merged with solvent peaks; LCMS (Method A): Rt=1.63 min, m/z=648.1 [M+H]⁺; HPLC (Method A): Rt=4.74 min, 99.51%; Chiral SFC (Method Q): Rt=2.04 min, 100%.

### Example 34. 5-Fluoro-N-isopropyl-N-((3R,4R)-4-methoxy-1-methylpyrrolidin-3-yl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 34)

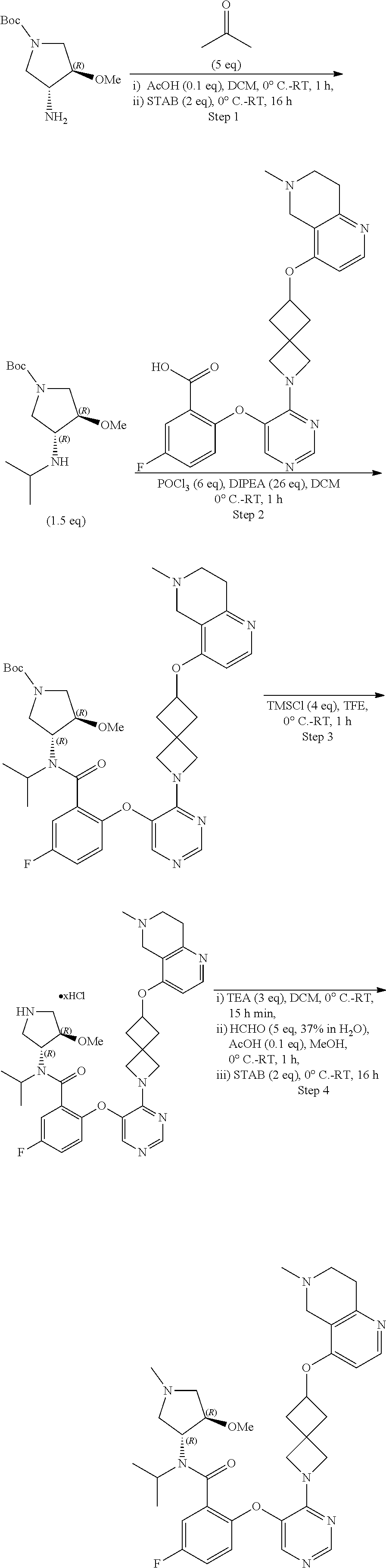

### Step 1. tert-butyl (3R,4R)-3-(isopropylamino)-4-methoxypyrrolidine-1-carboxylate

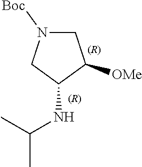

This compound was synthesized following the procedure described for the synthesis of Example 20, Step 1, starting with 1 equivalent of tert-butyl (3R,4R)-3-amino-4-methoxypyrrolidine-1-carboxylate, except that 5 equivalents of acetone and 2 equivalents of STAB were used, and DCM was used as the solvent. After completion, the reaction was quenched with saturated sodium bicarbonate solution and the mixture was extracted with DCM. The combined organic layer was dried over sodium sulfate and filtered, and the filtrate was concentrated under reduced to afford tert-butyl (3R,4R)-3-(isopropylamino)-4-methoxypyrrolidine-1-carboxylate as a sticky liquid. This material was used without further purification.

Yield: 84%; ¹H NMR (400 MHz, DMSO-d₆): δ 3.64-3.56 (m, 1H), 3.47-3.39 (m, 1H), 3.37-3.34 (m, 1H), 3.25 (s, 3H), 3.22-3.17 (m, 2H), 3.08-3.02 (m, 1H), 2.81-2.72 (m, 1H), 1.60-1.53 (m, 1H), 1.40 (s, 9H), 1.02-0.93 (m, 6H).

### Step 2. tert-Butyl (3R,4R)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-4-methoxypyrrolidine-1-carboxylate

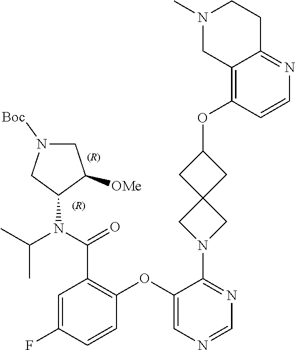

This compound was synthesized following the procedure described for the synthesis of Example 20, Step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.5 equivalents of tert-butyl (3R,4R)-3-(isopropylamino)-4-methoxypyrrolidine-1-carboxylate. After work-up, the crude was purified by reverse phase chromatography using a SiliCycle SiliaSep C18 column (40 g) on Biotage isolera one (Mobile phase A: 10 mM NH₄HCO₃ in water, B: ACN) to obtain tert-butyl (3R,4R)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-4-methoxypyrrolidine-1-carboxylate as a solid.

Yield: 26.6%; LCMS (Method A): Rt=1.93 min, m/z=732.2 [M+H]⁺, 88.70%.

### Step 3. 5-Fluoro-N-isopropyl-N-((3R,4R)-4-methoxypyrrolidin-3-yl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl

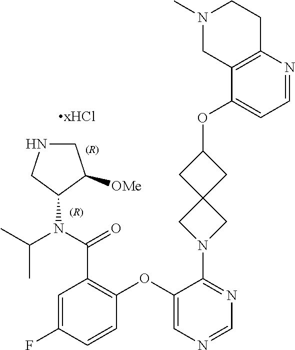

This compound was synthesized following the procedure described for the synthesis of Example 20, Step 3, starting with 1 equivalent of tert-butyl (3R,4R)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-4-methoxypyrrolidine-1-carboxylate. After completion, the reaction was concentrated under reduced pressure to afford crude 5-fluoro-N-isopropyl-N-((3R,4R)-4-methoxypyrrolidin-3-yl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl. This material was used without further purification.

Yield: 96%; LCMS (Method A): Rt=1.55 min, m/z=632.2 [M+H]⁺, 87.27%.

### Step 4. 5-Fluoro-N-isopropyl-N-((3R,4R)-4-methoxy-1-methylpyrrolidin-3-yl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

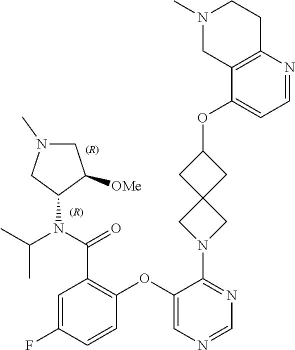

This compound was synthesized following the procedure described for the synthesis of Example 20, Step 4, starting with 1 equivalent of 5-fluoro-N-isopropyl-N-((3R,4R)-4-methoxypyrrolidin-3-yl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl, except that 3 equivalents of TEA, 0.1 equivalent of AcOH, 5 equivalents of formaldehyde (37% in H₂O), and 2 equivalents of STAB were used. After completion, the reaction was diluted with water and the mixture was extracted with 10% MeOH in DCM. The combined organic layer was washed with brine solution, dried over sodium sulfate, and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford 5-fluoro-N-isopropyl-N-((3R,4R)-4-methoxy-1-methylpyrrolidin-3-yl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 39.8%; ¹H NMR (400 MHz, DMSO-d₆): δ 8.31-8.27 (m, 1H), 8.21-8.16 (m, 1H), 7.80-7.69 (m, 1H), 7.39-7.21 (m, 2H), 7.16-7.01 (m, 1H), 6.69-6.61 (m, 1H), 4.84-4.69 (m, 1H), 4.34-4.07 (m, 5H), 3.85-3.71 (m, 1H), 3.61-3.50 (m, 1H), 3.38-3.35 (m, 2H), 3.28-3.10 (m, 3H), 2.91-2.72 (m, 6H), 2.66-2.56 (m, 4H), 2.36 (s, 3H), 2.31-2.11 (m, 5H), 1.55-0.95 (m, 6H); LCMS (Method A): Rt=1.64 min, m/z=646.2 [M+H]⁺; HPLC (Method A): Rt=4.66 min, 99.31%; Chiral SFC (Method W): Rt=2.40 min, 100%.

### Example 35. 5-Fluoro-N-isopropyl-N-((3R,4S)-4-methoxy-1-methylpyrrolidin-3-yl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 35)

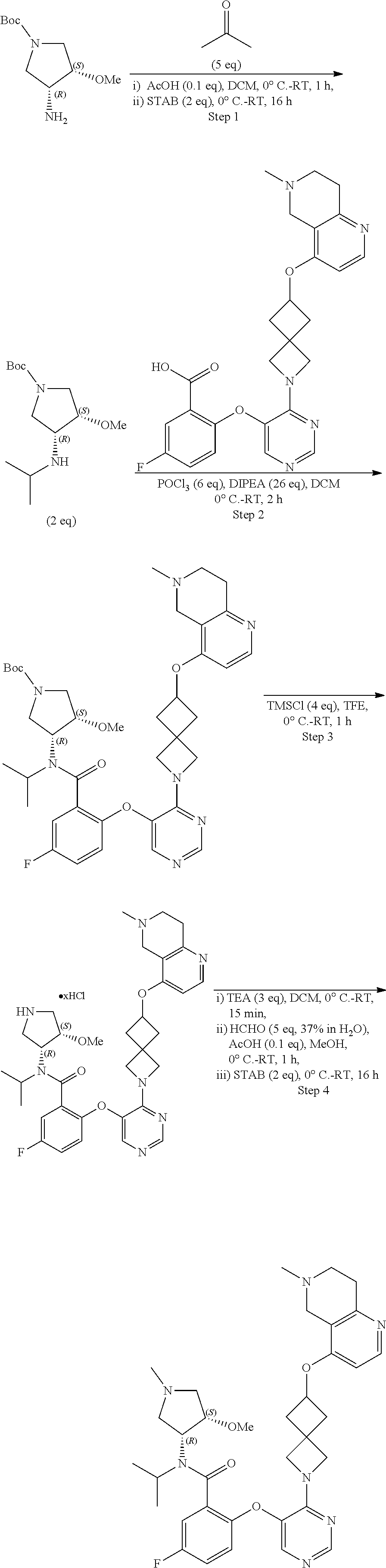

### Step 1. tert-Butyl (3R,4S)-3-(isopropylamino)-4-methoxypyrrolidine-1-carboxylate

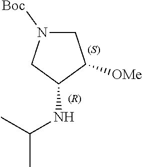

This compound was synthesized following the procedure described for the synthesis of Example 20, Step 1, starting with 1 equivalent of tert-butyl (3R,4S)-3-amino-4-methoxypyrrolidine-1-carboxylate, except that 5 equivalents of acetone and 2 equivalents of STAB were used, and DCM was used as the solvent. After completion, the reaction was quenched with saturated NaHCO₃ solution and the mixture was extracted with DCM. The combined organic layer was dried over sodium sulfate and filtered, and the filtrate was concentrated under reduced to afford tert-butyl (3R,4S)-3-(isopropylamino)-4-methoxypyrrolidine-1-carboxylate as a sticky liquid. This material was used without further purification.

Yield: 85%; ¹H NMR (400 MHz, DMSO-d₆): δ 3.79-3.68 (m, 1H), 3.45-3.34 (m, 3H), 3.29 (s, 3H), 3.26-3.17 (m, 1H), 2.86-2.72 (m, 2H), 1.73-1.55 (m, 1H), 1.39 (s, 9H), 1.01-0.95 (m, 6H); LCMS (Method A): Rt=1.67 min, m/z=259.0 [M+H]⁺, 91.92%.

### Step 2. tert-Butyl (3R,4S)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-4-methoxypyrrolidine-1-carboxylate

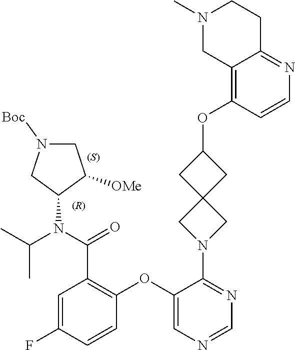

This compound was synthesized following the procedure described for the synthesis of Example 20, Step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 2 equivalents of tert-butyl (3R,4S)-3-(isopropylamino)-4-methoxypyrrolidine-1-carboxylate. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain tert-butyl (3R,4S)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-4-methoxypyrrolidine-1-carboxylate as a solid.

Yield: 93%; LCMS (Method A): Rt=2.00 min, m/z=732.2 [M+H]⁺, 96.86%.

### Step 3. 5-Fluoro-N-isopropyl-N-((3R,4S)-4-methoxypyrrolidin-3-yl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl

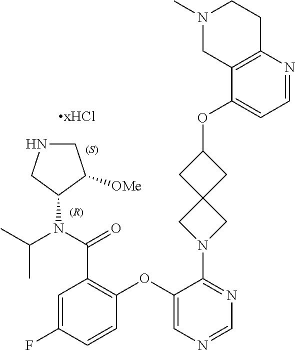

This compound was synthesized following the procedure described for the synthesis of Example 20, Step 3, starting with 1 equivalent of tert-butyl (3R,4S)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-4-methoxypyrrolidine-1-carboxylate. After completion, the reaction was concentrated under reduced pressure and re-concentrated from EtOAc to afford crude 5-fluoro-N-isopropyl-N-((3R,4S)-4-methoxypyrrolidin-3-yl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl. This material was used without further purification.

Yield: 99%; LCMS (Method A): Rt=1.59 min, m/z=632.1 [M+H]⁺, 97.45%.

### Step 4. 5-Fluoro-N-isopropyl-N-((3R,4S)-4-methoxy-1-methylpyrrolidin-3-yl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

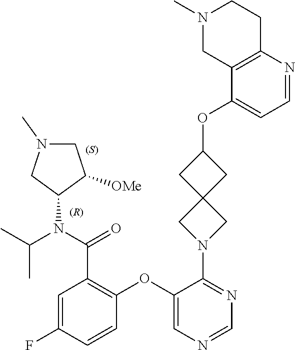

This compound was synthesized following the procedure described for the synthesis of Example 20, Step 4, starting with 1 equivalent of 5-fluoro-N-isopropyl-N-((3R,4S)-4-methoxypyrrolidin-3-yl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl, except that 3 equivalents of TEA, 0.1 equivalent of AcOH, 5 equivalents of formaldehyde (37% in H₂O), and 2 equivalents of STAB were used. After work-up, the crude was purified by reverse phase chromatography using a SiliCycle SiliaSep C18 column (80 g) on Biotage isolera one (Mobile phase A: 10 mM NH₄HCO₃₅ in water, B: ACN). The pure fractions were concentrated under reduced pressure and lyophilized to afford 5-fluoro-N-isopropyl-N-((3R,4S)-4-methoxy-1-methylpyrrolidin-3-yl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 55.5%; ¹H NMR (400 MHz, DMSO-d₆): δ 8.31-8.25 (m, 1H), 8.18 (d, J=5.5 Hz, 1H), 7.95-7.63 (m, 1H), 7.32-6.96 (m, 3H), 6.70-6.62 (m, 1H), 4.80-4.70 (m, 1H), 4.36-3.95 (m, 5H), 3.82-3.70 (m, 1H), 3.64-3.55 (m, 1H), 3.36 (br s, 2H), 3.17-2.99 (m, 2H), 2.89-2.73 (m, 5H), 2.65-2.55 (m, 4H), 2.42-2.31 (m, 4H), 2.31-2.13 (m, 6H), 1.55-0.98 (m, 6H); LCMS (Method A): Rt=1.66 min, m/z=646.2 [M+H]⁺; HPLC (Method A): Rt=4.59 min, 99.71%; Chiral SFC (Method AN): Rt=2.80 min, 100%.

### Example 36. 5-Fluoro-N-isopropyl-N-(2-methyl-2-azaspiro[3.3]heptan-6-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 36)

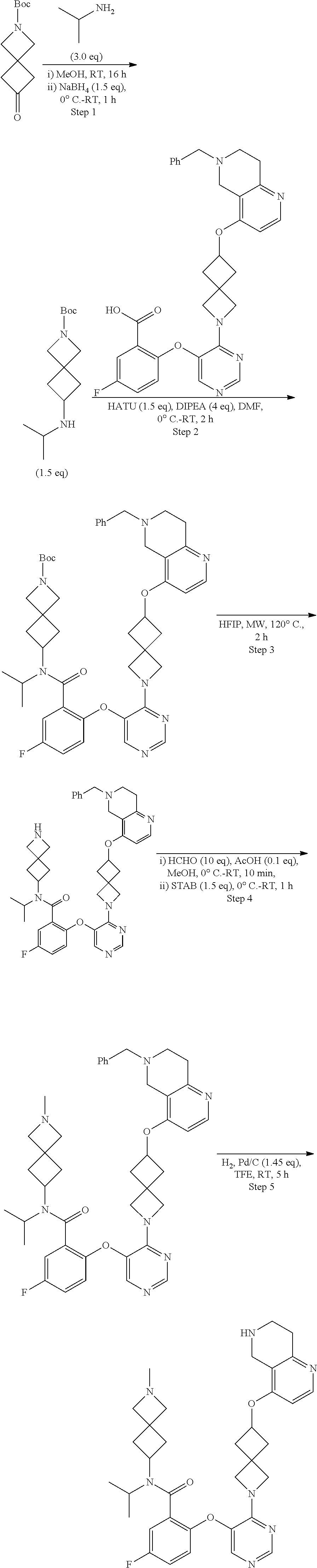

### Step 1. tert-Butyl 6-(isopropylamino)-2-azaspiro[3.3]heptane-2-carboxylate

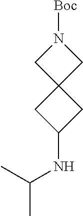 [SMILES: CC(C)NC1CC2(C1)CN(C(=O)OC(C)(C)C)C2]

To a stirred solution of tert-butyl 6-oxo-2-azaspiro[3.3]heptane-2-carboxylate (5.0 g, 23.67 mmol) in MeOH (50 mL), isopropylamine (4.20 g, 71.0 mmol) was added and the reaction was stirred at RT for 16 h. NaBH₄ (1.343 g, 35.5 mmol) was then added and the reaction was stirred at 0° C. for 1 h. The reaction progress was monitored by TLC (70% EtOAc in hexane). After completion, the reaction was concentrated under reduced pressure. The residue was diluted with water (50 mL), and the mixture was extracted with DCM (2×90 mL). The combined organic layer was dried over sodium sulfate and filtered, and the filtrate was concentrated under reduced pressure to obtain crude tert-butyl 6-(isopropylamino)-2-azaspiro[3.3]heptane-2-carboxylate (5.8 g, 96% yield) as a liquid. This material was used without further purification. ¹H NMR (400 MHz, DMSO-d₆): δ 3.82 (br s, 2H), 3.70 (br s, 2H), 3.16-2.99 (m, 1H), 2.71-2.59 (m, 1H), 2.38-2.27 (m, 2H), 1.72-1.81 (m, 2H), 1.53 (br dd, J=4.3, 9.3 Hz, 1H), 1.36 (s, 9H), 0.90 (d, J=6.3 Hz, 6H).

### Step 2. tert-Butyl 6-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-2-azaspiro[3.3]heptane-2-carboxylate

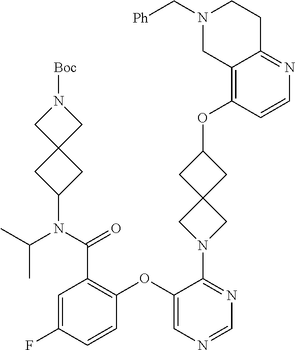 [SMILES: CC(C)N(C(=O)c1cc(F)ccc1Oc1cncnc1N1CC2(CC(Oc3ccnc4c3CN(Cc3ccccc3)CC4)C2)C1)C1CC2(C1)CN(C(=O)OC(C)(C)C)C2]

To a stirred solution of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid (500 mg, 0.881 mmol) and tert-butyl 6-(isopropylamino)-2-azaspiro[3.3]heptane-2-carboxylate (336 mg, 1.321 mmol) in DMF (10 mL), DIPEA (0.614 mL, 3.52 mmol) and HATU (502 mg, 1.321 mmol) were added at 0° C. The reaction was stirred at RT for 2 h. The reaction progress was monitored by TLC (10% MeOH in DCM). After completion, the reaction was concentrated under reduced pressure and the residue was diluted with water (40 mL). The mixture was extracted with EtOAc (2×70 mL). The combined organic layer was dried over sodium sulfate and filtered, and the filtrate was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN) to obtain tert-butyl 6-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-2-azaspiro[3.3]heptane-2-carboxylate (300 mg, 42.1% yield) as a solid. ¹HNMR (400 MHz, DMSO-d₆): δ 8.29-8.26 (m, 1H), 8.19 (d, J=5.6 Hz, 1H), 7.81-7.73 (m, 1H), 7.39-7.31 (m, 4H), 7.30-7.03 (m, 4H), 6.66 (d, J=5.6 Hz, 1H), 4.72 (quin, J=6.7 Hz, 1H), 4.43-4.03 (m, 4H), 3.95-3.78 (m, 4H), 3.75-3.70 (m, 1H), 3.68 (s, 2H), 3.66-3.61 (m, 1H), 3.47 (s, 2H), 2.96-2.73 (m, 5H), 2.72-2.67 (m, 2H), 2.43-2.35 (m, 1H), 2.28-2.16 (m, 3H), 1.45-1.22 (m, 12H), 1.09-0.87 (m, 4H); LCMS (Method A): Rt=2.37 min, m/z=804.5 [M+H]⁺, 99.35%.

### Step 3. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(2-azaspiro[3.3]heptan-6-yl)benzamide

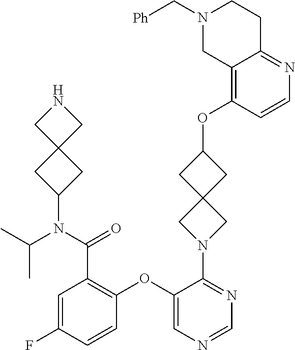 [SMILES: CC(C)N(C(=O)c1cc(F)ccc1Oc1cncnc1N1CC2(CC(Oc3ccnc4c3CN(Cc3ccccc3)CC4)C2)C1)C1CC2(CNC2)C1]

Step 3 was performed following the same procedure in two batches (25 mg and 150 mg).

In a 10 mL microwave vial, tert-butyl 6-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-2-azaspiro[3.3]heptane-2-carboxylate (150 mg, 0.187 mmol) was dissolved in HFIP (3 mL). The reaction was stirred at 120° C. under microwave for 2 h. The reaction progress was monitored by TLC (10% MeOH in DCM). After completion, the reaction was concentrated under reduced pressure. After workup, the crude reaction residue from both batches were combined and purified by reverse phase chromatography using a SiliCycle SiliaSep C18 column (80 g) on Biotage isolera one (Mobile phase A: 10 mM NH₄HCO₃ in water, B: ACN) to obtain 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(2-azaspiro[3.3]heptan-6-yl)benzamide (100 mg, 44.4% combined yield). ¹H NMR (400 MHz, DMSO-d₆): δ 8.32-8.24 (m, 1H), 8.19 (d, J=5.5 Hz, 1H), 7.78-7.67 (m, 1H), 7.42-7.01 (m, 8H), 6.66 (d, J=5.8 Hz, 11H), 4.72 (quin, J=6.6 Hz, 1H), 4.42-4.03 (m, 4H), 3.92-3.72 (m, 1H), 3.70-3.61 (m, 3H), 3.58-3.44 (m, 4H), 3.16-3.01 (m, 1H), 2.97-2.86 (m, 1H), 2.84-2.73 (m, 4H), 2.72-2.68 (m, 2H), 2.26-2.12 (m, 4H), 1.45-0.88 (m, 8H), one proton merged with solvent peaks; LCMS (Method A): Rt=2.03 min, m/z=704.2 [M+H]⁺, 92.27%.

### Step 4. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(2-methyl-2-azaspiro[3.3]heptan-6-yl)benzamide

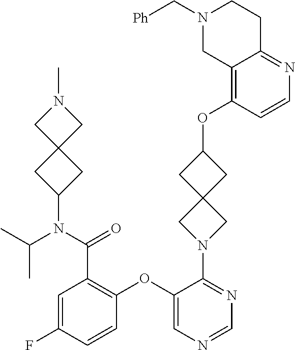 [SMILES: CC(C)N(C(=O)c1cc(F)ccc1Oc1cncnc1N1CC2(CC(Oc3ccnc4c3CN(Cc3ccccc3)CC4)C2)C1)C1CC2(C1)CN(C)C2]

To a stirred solution of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(2-azaspiro[3.3]heptan-6-yl)benzamide (50 mg, 0.071 mmol) in MeOH (1 mL), formaldehyde (0.071 mL, 0.710 mmol, 37% in H₂O) and AcOH (0.406 μL, 7.10 μmol) were added at 0° C. The reaction was stirred at RT for 10 min. To this reaction, STAB (22.58 mg, 0.107 mmol) was added at 0° C. and the reaction was stirred at RT for 1 h. The reaction progress was monitored by TLC (10% MeOH in DCM). After completion, 10% NaHCO₃ solution (10 mL) was added, and the mixture was extracted with EtOAc (2×15 mL). The combined organic layer was dried over anhydrous sodium sulfate and filtered. The filtrate was concentrated under reduced pressure to afford crude 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(2-methyl-2-azaspiro[3.3]heptan-6-yl)benzamide (45 mg, 86% yield) as a semi-solid. This material was used without further purification. ¹H NMR (400 MHz, DMSO-d₆): δ 8.31-8.25 (m, 1H), 8.19 (d, J=5.7 Hz, 1H), 7.78-7.68 (m, 1H), 7.41-7.21 (m, 7H), 7.18-7.02 (m, 1H), 6.66 (d, J=5.8 Hz, 11H), 4.79-4.66 (m, 1H), 4.34-4.03 (m, 4H), 3.94-3.75 (m, 1H), 3.68 (s, 2H), 3.47 (s, 2H), 3.20-3.03 (m, 2H), 2.99-2.88 (m, 2H), 2.84-2.73 (m, 4H), 2.72-2.66 (m, 2H), 2.28-2.18 (m, 3H), 2.14-2.05 (m, 4H), 1.46-1.32 (m, 2H), 1.29-0.89 (m, 6H); LCMS (Method C): Rt=1.62 min, m/z=718.3 [M+H]⁺, 97.45%.

### Step 5. 5-Fluoro-N-isopropyl-N-(2-methyl-2-azaspiro[3.3]heptan-6-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

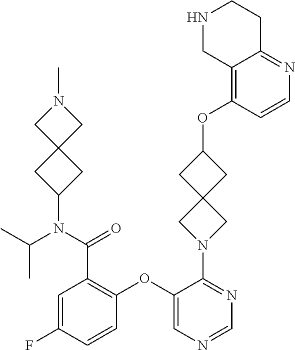 [SMILES: CC(C)N(C(=O)c1cc(F)ccc1Oc1cncnc1N1CC2(CC(Oc3ccnc4c3CNCC4)C2)C1)C1CC2(C1)CN(C)C2]

In a 25 mL round bottom flask, 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(2-methyl-2-azaspiro[3.3]heptan-6-yl)benzamide (40 mg, 0.056 mmol) was dissolved in TFE (1 mL). To this solution, Pd—C (83 mg, 0.081 mmol) was added and the reaction was stirred at RT for 5 h under a hydrogen atmosphere (balloon pressure). The reaction progress was monitored by TLC (10/0 MeOH in DCM). After completion, the reaction was filtered through a Celite® pad, and the pad was washed with MeOH (50 mL). The filtrate was concentrated under reduced pressure. The crude product was purified by reverse phase chromatography using a SiliCycle SiliaSep C18 column (12 g) on Biotage isolera one (Mobile phase A: 10 mM NH₄HCO₃ in water, B: ACN) to afford 5-fluoro-N-isopropyl-N-(2-methyl-2-azaspiro[3.3]heptan-6-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (2.5 mg, 6.86% yield) as a solid. ¹H NMR (400 MHz, DMSO-d₆): δ 8.31-8.24 (m, 1H), 8.21-8.12 (m, 1H), 7.78-7.68 (m, 1H), 7.32-7.02 (m, 3H), 6.69-6.59 (m, 1H), 4.73 (quin, J=6.5 Hz, 1H), 4.42-4.08 (m, 4H), 3.90-3.74 (m, 1H), 3.73-3.49 (m, 3H), 3.19-3.04 (m, 3H), 2.99-2.91 (m, 4H), 2.86-2.74 (m, 3H), 2.71-2.64 (m, 2H), 2.30-2.18 (m, 3H), 2.16-2.04 (m, 4H), 1.49-0.89 (m, 6H), one proton merged with solvent peaks; LCMS (Method C): Rt=1.50 min, m/z=628.3 [M+H]⁺; HPLC (Method B): Rt=2.48 min, 95.96%.

### Example 37. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(2-methyl-2-azaspiro[3.3]heptan-6-yl)benzamide (Compound No. 37)

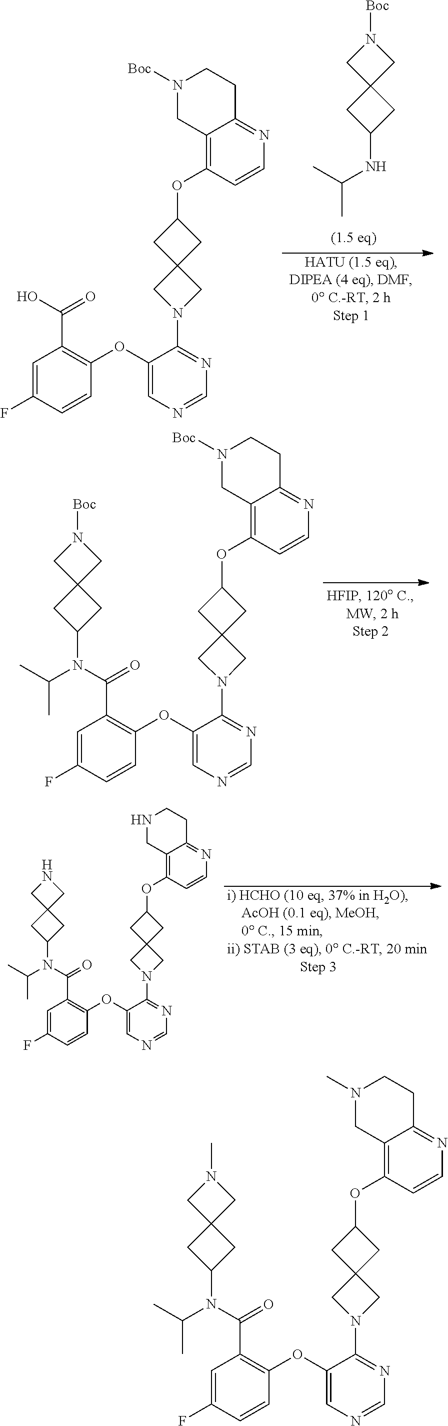

### Step 1. tert-Butyl 4-((2-(5-(2-((2-(tert-butoxycarbonyl)-2-azaspiro[3.3]heptan-6-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate

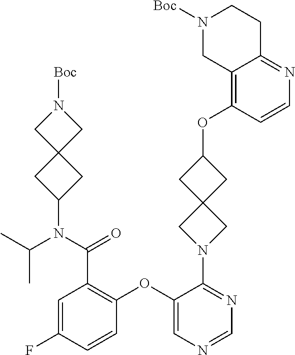 [SMILES: CC(C)N(C(=O)c1cc(F)ccc1Oc1cncnc1N1CC2(CC(Oc3ccnc4c3CN(C(=O)OC(C)(C)C)CC4)C2)C1)C1CC2(C1)CN(C(=O)OC(C)(C)C)C2]

This compound was synthesized following the procedure described for the synthesis of Example 36, Step 2, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.5 equivalents of tert-butyl 6-(isopropylamino)-2-azaspiro[3.3]heptane-2-carboxylate. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl 4-((2-(5-(2-((2-(tert-butoxycarbonyl)-2-azaspiro[3.3]heptan-6-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate as a solid.

Yield: 82%; ¹H NMR (400 MHz, DMSO-d₆): δ 8.29-8.27 (m, 1H), 8.25 (d, J=5.6 Hz, 1H), 7.82-7.72 (m, 1H), 7.34-7.04 (m, 3H), 6.77 (d, J=5.8 Hz, 1H), 4.79 (quin, J=6.6 Hz, 1H), 4.37 (br s, 2H), 4.34-4.09 (m, 4H), 3.96-3.78 (m, 4H), 3.75-3.64 (m, 2H), 3.62 (br t, J=5.9 Hz, 2H), 3.16-3.03 (m, 1H), 2.88-2.75 (m, 4H), 2.43-2.18 (m, 4H), 1.43 (s, 9H), 1.33 (s, 9H), 1.29-1.24 (m, 2H), 1.09-0.89 (m, 4H), one proton merged with solvent peaks; LCMS (Method A): Rt=2.29 min, m/z=814.4 [M+H]⁺, 99.12%.

### Step 2. 5-Fluoro-N-isopropyl-N-(2-azaspiro[3.3]heptan-6-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

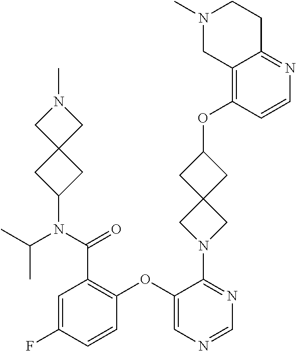 [SMILES: CC(C)N(C(=O)c1cc(F)ccc1Oc1cncnc1N1CC2(CC(Oc3ccnc4c3CN(C)CC4)C2)C1)C1CC2(C1)CN(C)C2]

This compound was synthesized following the procedure described for the synthesis of Example 36, Step 3, starting with 1 equivalent of tert-butyl 4-((2-(5-(2-((2-(tert-butoxycarbonyl)-2-azaspiro[3.3]heptan-6-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate. The crude was purified by Prep HPLC (Method H) to afford 5-fluoro-N-isopropyl-N-(2-azaspiro[3.3]heptan-6-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 32.5%. ¹H NMR (400 MHz, DMSO-d₆): δ 8.36-8.24 (m, 1H), 8.16 (d, J=5.6 Hz, 1H), 7.79-7.68 (m, 1H), 7.33-7.01 (m, 3H), 6.64 (d, J=5.6 Hz, 1H), 4.73 (quin, J=6.4 Hz, 1H), 4.39-4.06 (m, 4H), 3.90-3.61 (m, 6H), 3.54-3.45 (m, 3H), 3.02-2.89 (m, 3H), 2.85-2.73 (m, 2H), 2.68 (br t, J=5.4 Hz, 2H), 2.31-2.11 (m, 4H), 1.46-0.91 (m, 6H), two protons merged with solvent peaks; LCMS (Method B): Rt=0.81 min, m/z=614.3 [M+H]⁺, 94.31%.

### Step 3. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(2-methyl-2-azaspiro[3.3]heptan-6-yl)benzamide

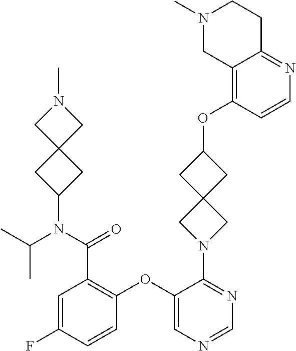 [SMILES: CC(C)N(C(=O)c1cc(F)ccc1Oc1cncnc1N1CC2(CC(Oc3ccnc4c3CN(C)CC4)C2)C1)C1CC2(C1)CN(C)C2]

This compound was synthesized following the procedure described for the synthesis of Example 36, Step 4, starting with 1 equivalent of 5-fluoro-N-isopropyl-N-(2-azaspiro[3.3]heptan-6-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide, except that 3 equivalents of STAB was used. After work-up, the crude was purified by reverse phase chromatography using a SiliCycle SiliaSep C18 column (40 g) on Biotage isolera one (Mobile phase A: 10 mM NH₄HCO₃ in water, B: ACN) to obtain 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(2-methyl-2-azaspiro[3.3]heptan-6-yl)benzamide as a solid.

Yield: 33.2% yield; ¹H NMR (400 MHz, DMSO-d₆): δ 8.32-8.25 (m, 1H), 8.18 (d, J=5.5 Hz, 1H), 7.78-7.67 (m, 1H), 7.33-7.03 (m, 3H), 6.66 (d, J=5.8 Hz, 1H), 4.81-4.70 (m, 1H), 4.38-4.07 (m, 4H), 3.94-3.74 (m, 1H), 3.71-3.60 (m, 1H), 3.37 (s, 2H), 3.17-3.03 (m, 3H), 3.00-2.90 (m, 2H), 2.85-2.75 (m, 4H), 2.63 (br t, J=5.8 Hz, 2H), 2.36 (s, 3H), 2.30-2.22 (m, 2H), 2.21-2.04 (m, 5H), 2.01-1.84 (m, 1H), 1.47-0.89 (m, 6H); LCMS (Method B): Rt=0.92 min, m/z=642.3 [M+H]⁺, HPLC (Method A): Rt=4.48 min, 99.34%.

### Example 38. (E1)-5-Fluoro-N-isopropyl-N-(1-methyl-5-oxopyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 38)

### Example 39. (E2)-5-Fluoro-N-isopropyl-N-(1-methyl-5-oxopyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 39)

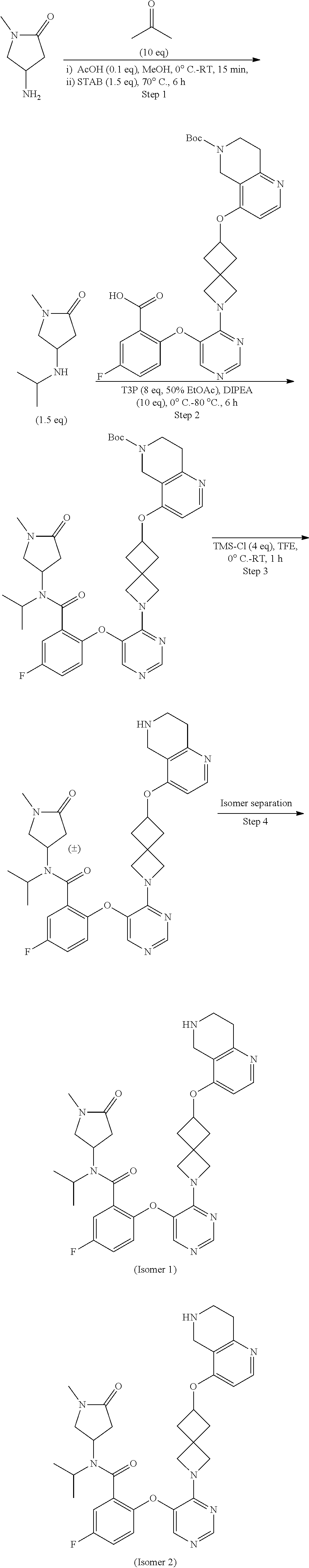

### Step 1. (±)-4-(Isopropylamino)-1-methylpyrrolidin-2-one

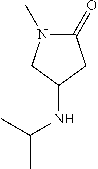

This compound was synthesized following the procedure described for the synthesis of Example 20, Step 1, starting with 1 equivalent of (±)-4-amino-1-methylpyrrolidin-2-one, except that 10 equivalents of acetone was used. The reaction was stirred at 70° C. for 5 h. After completion, the reaction was concentrated under reduced pressure and EtOAc was added to the residue. The resulting mixture was filtered through a sintered funnel. The filtrate was concentrated under reduced pressure and 10% NaOH solution was added to the residue. The mixture was extracted with 10% MeOH in DCM. The combined organic layer was dried over anhydrous sodium sulfate and filtered, and the filtrate was concentrated under reduced pressure to afford crude (±)-4-(isopropylamino)-1-methylpyrrolidin-2-one as a liquid. This material was used without further purification.

Yield: 73.1%; ¹H NMR (400 MHz, DMSO-d₆) δ=3.52-3.39 (m, 2H), 3.01 (dd, J=4.4, 9.0 Hz, 1H), 2.80-2.71 (m, 1H), 2.69 (s, 3H), 2.41 (dd, J=7.4, 16.5 Hz, 1H), 1.99 (dd, J=5.5, 16.4 Hz, 1H), 1.81 (br s, 1H), 1.01-0.93 (m, 6H).

### Step 2. (±)-tert-Butyl 4-((2-(5-(4-fluoro-2-(isopropyl(1-methyl-5-oxopyrrolidin-3-yl)carbamoyl)phenoxyl)pyrimidin-4-yl)-2-azaspiro[3.3]heptan-6-yl)oxy)-7,8-dihydro-1,6-naphthridine-6(5H)-carboxylate

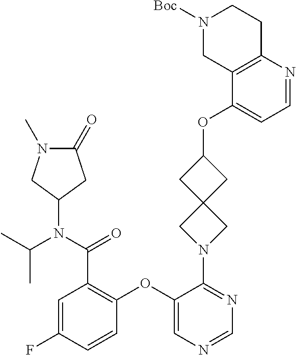 [SMILES: CC(C)N(C(=O)c1cc(F)ccc1Oc1cncnc1N1CC2(CC(Oc3ccnc4c3CN(C(=O)OC(C)(C)C)CC4)C2)C1)C1CC(=O)N(C)C1]

This compound was synthesized following the procedure described for the synthesis of Example 25, Step 1, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.5 equivalents of (±)-4-(isopropylamino)-1-methylpyrrolidin-2-one was used, except that 8 equivalents of T3P (50% in EtOAc) and 10 equivalents of DIPEA were used. After completion, the reaction was diluted with 10% NaHCO₃ solution and the mixture was extracted with EtOAc. The combined organic layer was dried over anhydrous sodium sulfate and filtered, and the filtrate was concentrated under reduced pressure. The crude product was purified by reverse phase chromatography using a SiliCycle SiliaSep C18 column (12 g) on Biotage isolera one (Mobile phase A: 10 mM NH₄HCO₃ in water, B: ACN) to afford (±)-tert-butyl 4-((2-(5-(4-fluoro-2-(isopropyl(1-methyl-5-oxopyrrolidin-3-yl)carbamoyl)phenoxyl)pyrimidin-4-yl)-2-azaspiro[3.3]heptan-6-yl)oxy)-7,8-dihydro-1,6-naphthyridine-6(5H)-carboxylate as a solid.

Yield: 52.5% yield; ¹H NMR (400 MHz, DMSO-d₆): δ 8.35-8.27 (m, 1H), 8.23 (d, J=5.6 Hz, 1H), 7.82-7.73 (m, 1H), 7.41-7.34 (m, 1H), 7.31-7.23 (m, 1H), 7.11-6.97 (m, 1H), 6.79-6.71 (m, 1H), 4.85-4.71 (m, 1H), 4.36 (s, 2H), 4.32-4.25 (m, 1H), 4.24-4.10 (m, 4H), 3.79-3.69 (m, 1H), 3.65-3.49 (m, 4H), 2.87-2.76 (m, 5H), 2.73-2.63 (m, 3H), 2.32-2.22 (m, 2H), 1.43 (s, 9H), 1.14-0.98 (m, 6H), one proton merged with solvent peaks; LCMS (Method A): Rt=1.81 min, m/z=716.1 [M+H]⁺, 99.67%.

### Step 3. (±)-5-Fluoro-N-isopropyl-N-(1-methyl-5-oxopyrrolidine-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

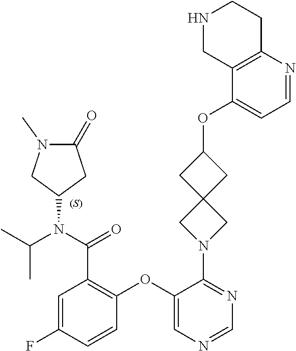

This compound was synthesized following the procedure described for the synthesis of Example 20, Step 3, starting with 1 equivalent of (±)-tert-butyl 4-((2-(5-(4-fluoro-2-(isopropyl(1-methyl-5-oxopyrrolidin-3-yl)carbamoyl)phenoxyl)pyrimidin-4-yl)-2-azaspiro[3.3]heptan-6-yl)oxy)-7,8-dihydro-1,6-naphthyridine-6(5H)-carboxylate. After completion, the reaction was concentrated under reduced pressure. To this crude product, 10% NaHCO₃ solution was added and the mixture was extracted with 10% MeOH in DCM. The combined organic layer was dried over sodium sulfate and filtered, and the filtrate was concentrated under reduced pressure to obtain crude (±)-5-fluoro-N-isopropyl-N-(1-methyl-5-oxopyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid. This material was used without further purification. Quantitative yield; LCMS (Method A): Rt=1.42 min, m/z=616.1 [M+H]⁺, 98.53%.

### Step 4. (E1)-5-Fluoro-N-isopropyl-N-(1-methyl-5-oxopyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

and

### (E2)-5-fluoro-N-isopropyl-N-(1-methyl-5-oxopyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

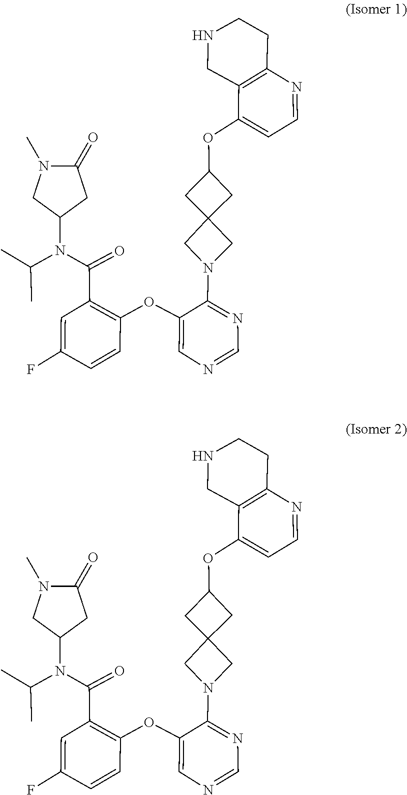 [SMILES: CC(C)N(C(=O)c1cc(F)ccc1Oc1cncnc1N1CC2(CC(Oc3ccnc4c3CNCC4)C2)C1)C1CC(=O)N(C)C1 ; CC(C)N(C(=O)c1cc(F)ccc1Oc1cncnc1N1CC2(CC(Oc3ccnc4c3CNCC4)C2)C1)C1CC(=O)N(C)C1]

(±)-5-Fluoro-N-isopropyl-N-(1-methyl-5-oxopyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (235 mg, 0.38 mmol) was purified by chiral Prep SFC (Method F) to obtain both isomers. These isomers were impure, therefore they were each re-purified separately by Prep HPLC (Method A) to obtain (E1)-5-fluoro-N-isopropyl-N-(1-methyl-5-oxopyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (32 mg, 17.56%) and (E2)-5-fluoro-N-isopropyl-N-(1-methyl-5-oxopyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (43 mg, 23.72%) as solids.

Isomer 1. ¹H NMR (400 MHz, DMSO-d₆): δ 8.36-8.28 (m, 1H), 8.16 (d, J=5.6 Hz, 1H), 7.83-7.74 (m, 1H), 7.42-7.32 (m, 1H), 7.31-7.22 (m, 1H), 7.10-6.97 (m, 1H), 6.72-6.60 (m, 1H), 4.80-4.64 (m, 1H), 4.36-4.05 (m, 5H), 3.80-3.72 (m, 1H), 3.69 (s, 2H), 3.63-3.49 (m, 2H), 3.41-3.37 (m, 11H), 2.95 (br t, J=5.8 Hz, 2H), 2.84-2.72 (m, 4H), 2.71-2.64 (m, 4H), 2.28-2.18 (m, 2H), 1.16-0.95 (m, 6H), one proton merged with solvent peaks; LCMS (Method A): Rt=1.43 min, m/z=616.10 [M+H]⁺; HPLC (Method A): Rt=4.01 min, 92.75%; Chiral SFC (Method Y): Rt=6.43 min, 100%.

Isomer 2. ¹H NMR (400 MHz, DMSO-d₆): δ 8.34-8.28 (m, 1H), 8.16 (d, J=5.6 Hz, 1H), 7.83-7.73 (m, 1H), 7.43-7.34 (m, 1H), 7.31-7.20 (m, 1H), 7.10-6.90 (m, 1H), 6.71-6.61 (m, 1H), 4.81-4.67 (m, 1H), 4.34-4.07 (m, 5H), 3.80-3.73 (m, 1H), 3.71 (s, 2H), 3.64-3.48 (m, 2H), 3.41-3.36 (m, 1H), 2.96 (t, J=5.8 Hz, 2H), 2.84-2.72 (m, 4H), 2.71-2.65 (m, 4H), 2.30-2.18 (m, 2H), 1.18-0.95 (m, 6H), one proton merged with solvent peaks; LCMS (Method A): Rt=1.43 min, m/z=616.20 [M+H]⁺; HPLC (Method A): Rt=4.01 min, 99.64%; Chiral SFC (Method Y): Rt=9.85 min, 98.69%.

### Example 40. N-((1r,3r)-3-(Dimethylamino)-3-methylcyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 40)

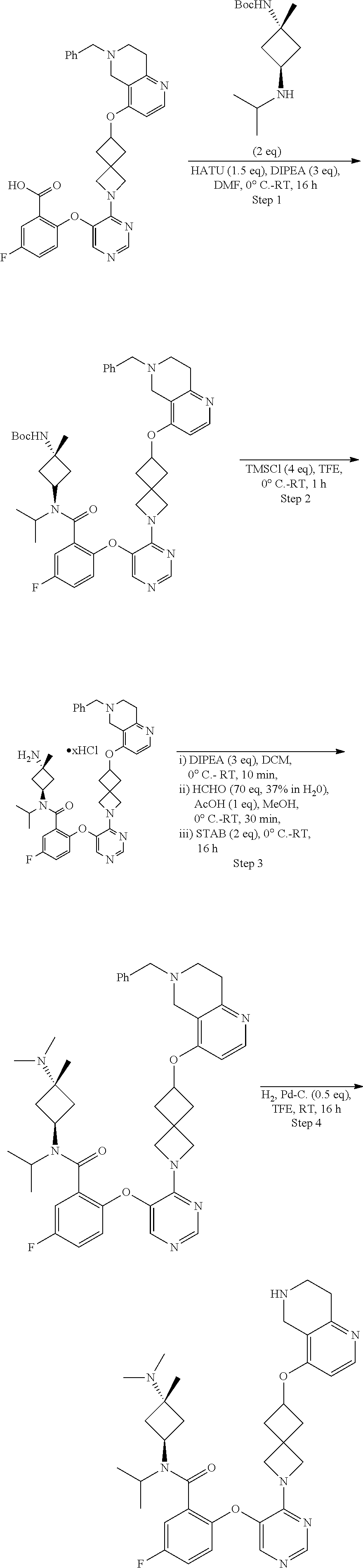

### Step 1. tert-Butyl ((1r,3r)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-1-methylcyclobutyl)carbamate

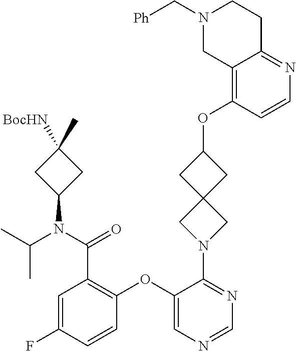 [SMILES: CC(C)N(C(=O)c1cc(F)ccc1Oc1cncnc1N1CC2(CC(Oc3ccnc4c3CN(Cc3ccccc3)CC4)C2)C1)[C@H]1C[C@](C)(NC(=O)OC(C)(C)C)C1]

This compound was synthesized following the procedure described for the synthesis of Example 1, step 2, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 2 equivalents of tert-butyl ((1r,3r)-3-(isopropylamino)-1-methylcyclobutyl)carbamate. After work-up, the crude was purified by silica gel flash column chromatography using MeOH in DCM (product eluted at 10% MeOH in DCM) to obtain tert-butyl ((1r,3r)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-1-methylcyclobutyl)carbamate as a solid.

Yield: 59.9%; LCMS (Method B): Rt=1.50 min, m/z=792.2 [M+H]⁺, 91.16%.

### Step 2. N-((1r,3r)-3-Amino-3-methylcyclobutyl)-2-((4-(6-((6-ben yl-5,6,7,8-tetrahydro-1,6-naphthyridin-4-yl)oxy)-2-azaspiro[3.3]heptan-2-yl)pyrimidin-5- yl)oxy)-5-fluoro-N-isopropylbenzamide xHCl

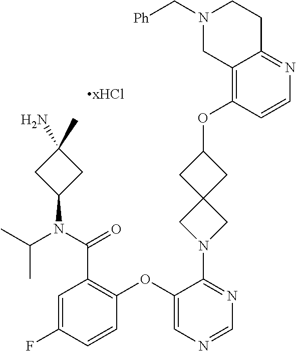 [SMILES: CC(C)N(C(=O)c1cc(F)ccc1Oc1cncnc1N1CC2(CC(Oc3ccnc4c3CN(Cc3ccccc3)CC4)C2)C1)[C@H]1C[C@](C)(N)C1]

This compound was synthesized following the procedure described for the synthesis of Example 20, step 2, starting with 1 equivalent of tert-butyl ((1r,3r)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-1-methylcyclobutyl)carbamate. After completion, the reaction was concentrated under reduced pressure to obtain crude N-((1r,3r)-3-amino-3-methylcyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl as a solid. This material was used without further purification.

Yield: 98%; LCMS (Method A): Rt=2.01 min, m/z=692.3 [M+H]⁺, 98.77%.

### Step 3. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide

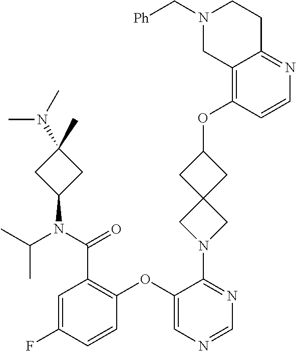 [SMILES: CC(C)N(C(=O)c1cc(F)ccc1Oc1cncnc1N1CC2(CC(Oc3ccnc4c3CN(Cc3ccccc3)CC4)C2)C1)[C@H]1C[C@](C)(N(C)C)C1]

This compound was synthesized following the procedure described for the synthesis of Example 10, step 2, starting with 1 equivalent of N-((1r,3r)-3-amino-3-methylcyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl, except that 70 equivalents of formaldehyde (37% in H₂O) was used, and MeOH was used as the solvent. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide as a solid.

Yield: 63.7%; LCMS (Method A): Rt=2.11 min, m/z=720.4 [M+H]⁺, 98.67%.

### Example 41. N-((1s,3s)-3-(Dimethylamino)-3-methylcyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 41)

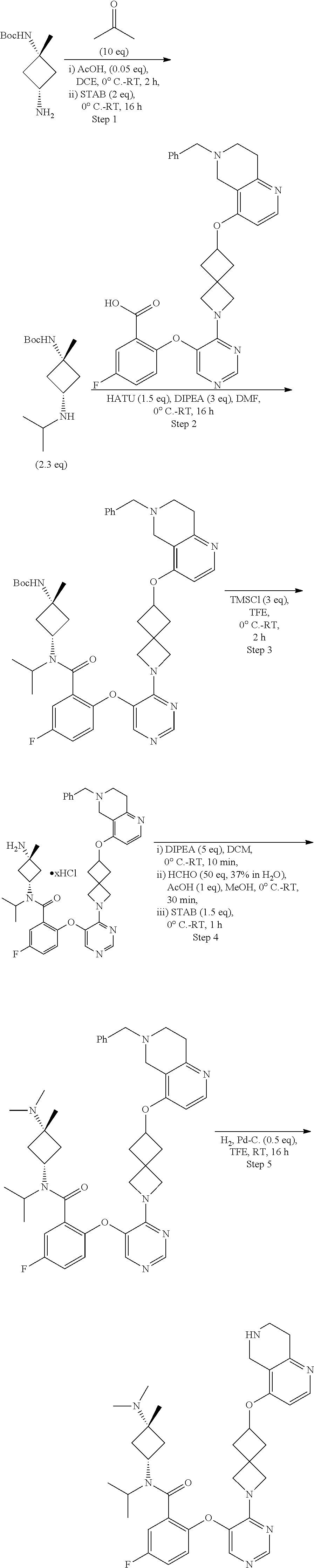

### Step 1. tert-Butyl ((1s,3s)-3-(isopropylamino)-1-methylcyclobutyl)carbamate

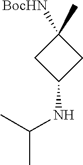 [SMILES: CC(C)N[C@H]1C[C@@](C)(NC(=O)OC(C)(C)C)C1]

This compound was synthesized following the procedure described for the synthesis of Intermediate 20, starting with 1 equivalent of tert-butyl ((1s,3s)-3-amino-1-methylcyclobutyl)carbamate, except that 10 equivalents of acetone, 0.05 equivalent of AcOH, 1.5 equivalents of STAB were used, and DCE was used as the solvent. After completion, the reaction was quenched with saturated sodium bicarbonate solution and the mixture was extracted with DCM. The combined organic layer was dried over sodium sulfate and filtered. The filtrate was concentrated under reduced pressure to obtain crude tert-butyl ((1s,3s)-3-(isopropylamino)-1-methylcyclobutyl)carbamate as a semi-solid. This material was used without further purification.

Yield: 96%; ¹H NMR (400 MHz, DMSO-d₆): δ 6.96-6.89 (br s, 1H), 3.01 (quin, J=7.7 Hz, 1H), 2.74-2.61 (m, 1H), 2.21-2.12 (m, 2H), 1.90-1.76 (m, 2H), 1.37 (s, 9H), 1.26 (s, 3H), 0.92 (d, J=6.2 Hz, 6H), one proton merged with solvent peaks.

### Step 2. tert-Butyl ((1s,3s)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-1-methylcyclobutyl)carbamate

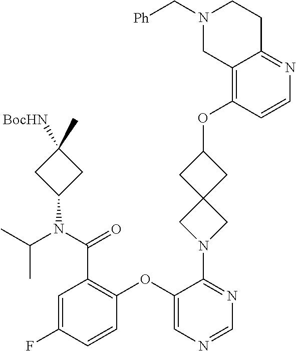 [SMILES: CC(C)N(C(=O)c1cc(F)ccc1Oc1cncnc1N1CC2(CC(Oc3ccnc4c3CN(Cc3ccccc3)CC4)C2)C1)[C@H]1C[C@@](C)(NC(=O)OC(C)(C)C)C1]

This compound was synthesized following the procedure described for the synthesis of Example 1, step 2, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 2.3 equivalents of tert-butyl ((1s,3s)-3-(isopropylamino)-1-methylcyclobutyl)carbamate. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl ((1s,3s)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-1-methylcyclobutyl)carbamate as a solid.

Yield: 76%; LCMS (Method A): Rt=2.29 min, m/z=792.5 [M+H]⁺, 71.14%.

### Step 3. 2-((4-(6-((6-Benzyl-5,6,7,8-tetrahydro-1,6-naphthyridin-4-y)oxy)-2-azaspiro[3.3]heptan-2-yl)pyrimidin-5-yl)oxy)-N-((1s,3s)-3-(dimethylamino)-3-methylcyclobutyl)-5-fluoro-N-isopropylbenzamide xHCl

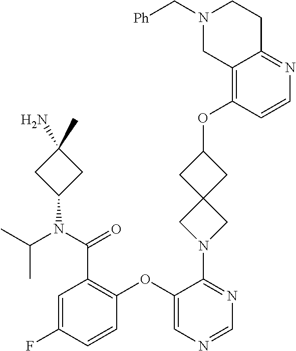 [SMILES: CC(C)N(C(=O)c1cc(F)ccc1Oc1cncnc1N1CC2(CC(Oc3ccnc4c3CN(Cc3ccccc3)CC4)C2)C1)[C@H]1C[C@@](C)(N)C1]

This compound was synthesized following the procedure described for the synthesis of Example 20, Step 3, starting with 1 equivalent of tert-butyl ((1s,3s)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-1-methylcyclobutyl)carbamate, except that 3 equivalents of TMSCl was used. After completion, the reaction was concentrated under reduced pressure to obtain crude N-((1s,3s)-3-amino-3-methylcyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl. This material was used without further purification.

Yield: 97%; LCMS (Method A): Rt=2.13 min, m/z=692.3 [M+H]⁺, 96.47%.

### Step 4. 2-((4-(6-((6-Benzyl-5,6,7,8-tetrahydro-,6-naphthyridin-4-yl)oxy)-2-azaspiro[3.3]heptan-2-yl)pyrimidin-5-yl)oxy)-N-((1s,3s)-3-(dimethylamino)-3-methylcyclobutyl)-5-fluoro-N-isopropylbenzamide

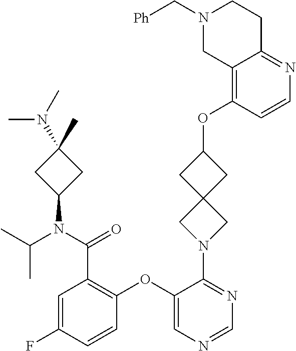 [SMILES: CC(C)N(C(=O)c1cc(F)ccc1Oc1cncnc1N1CC2(CC(Oc3ccnc4c3CN(Cc3ccccc3)CC4)C2)C1)[C@H]1C[C@](C)(N(C)C)C1]

This compound was synthesized following the procedure described for the synthesis of Example 20, step 4, starting with 1 equivalent of N-((1s,3s)-3-amino-3-methylcyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl, except that 5 equivalents of DIPEA, 50 equivalents of formaldehyde (37% in H₂O), 1 equivalent of AcOH, and 3 equivalents of STAB were used. After work-up, the crude was purified by reverse phase chromatography using a SiliCycle SiliaSep C18 column (40 g) on Biotage isolera one (Mobile phase A: 10 mM NH₄HCO₃ in water, B: ACN) to obtain 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1s,3s)-3-(dimethylamino)-3-methylcyclobutyl)-5-fluoro-N-isopropylbenzamide as a solid.

Yield: 72.3%; LCMS (Method A): Rt=2.15 min, m/z=720.3 [M+H]⁺, 97.99%.

### Step 5. N-((1s,3s)-3-(Dimethylamino)-3-methylcyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

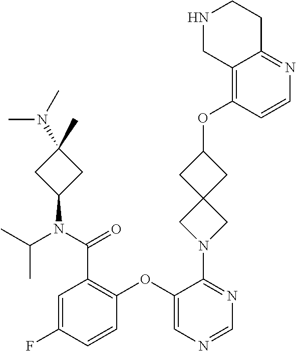 [SMILES: CC(C)N(C(=O)c1cc(F)ccc1Oc1cncnc1N1CC2(CC(Oc3ccnc4c3CNCC4)C2)C1)[C@H]1C[C@](C)(N(C)C)C1]

This compound was synthesized following the procedure described for the synthesis of Example 20, step 5, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1s,3s)-3-(dimethylamino)-3-methylcyclobutyl)-5-fluoro-N-isopropylbenzamide, except that 0.5 equivalent of Pd-C was used. After filtration and concentration, the crude was purified by reverse phase chromatography using a SiliCycle SiliaSep C18 column (80 g) on Biotage isolera one (Mobile phase A: 10 mM NH₄HCO₃ in water, B: ACN) to obtain N-((1s,3s)-3-(dimethylamino)-3-methylcyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide.

Yield: 47.7%; ¹H NMR (400 MHz, DMSO-d₆): δ 8.31-8.23 (m, 1H), 8.16 (d, J=5.6 Hz, 1H), 7.78-7.66 (m, 1H), 7.32-7.21 (m, 1H), 7.18-7.02 (m, 2H), 6.63 (d, J=5.6 Hz, 1H), 4.79-4.66 (m, 1H), 4.35-4.09 (m, 4H), 3.87-3.64 (m, 4H), 2.94 (t, J=5.6 Hz, 2H), 2.87-2.73 (m, 2H), 2.71-2.67 (m, 2H), 2.29-2.17 (m, 2H), 2.04-1.83 (m, 9H), 1.75-1.65 (m, 1H), 1.47-0.80 (m, 9H), one proton merged with solvent peaks; LCMS (Method C): Rt=1.17 min, m/z=630.2 [M+H]⁺; HPLC (Method A): Rt=4.34 min, 99.30%/; Chiral SFC (Method U): Rt=1.10 min, 100%.

### Example 42. N-((1s,3s)-3-(Dimethylamino)-1-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 42)

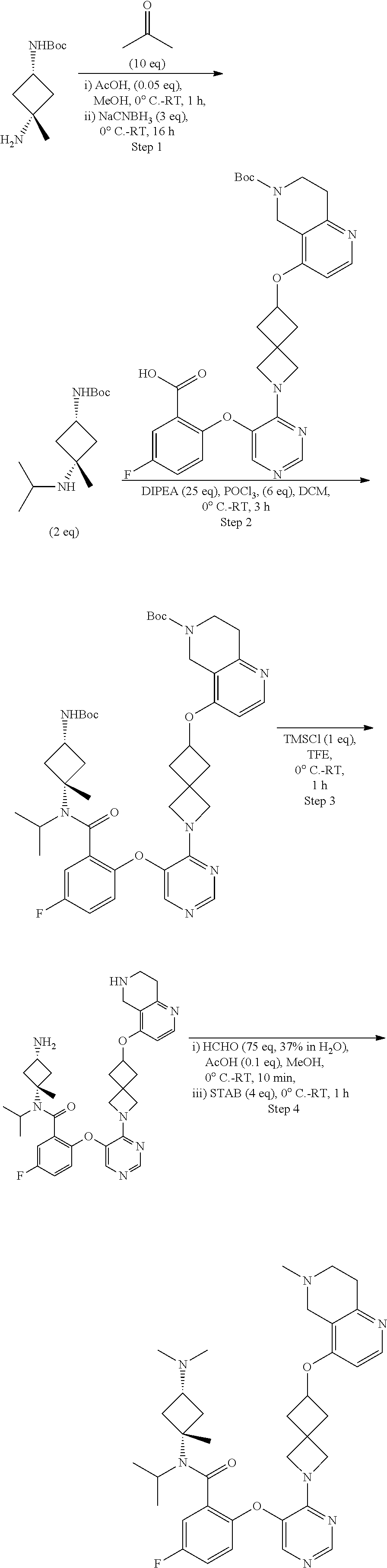

### Step 1. tert-Butyl ((1s,3s)-3-(isopropylamino)-3-methylcyclobutyl)carbamate

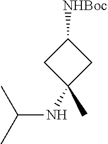 [SMILES: CC(C)N[C@]1(C)C[C@H](NC(=O)OC(C)(C)C)C1]

In a dried, round bottom flask under a nitrogen atmosphere, tert-butyl ((1s,3s)-3-amino-3-methylcyclobutyl)carbamate (0.800 g, 3.99 mmol) was dissolved in MeOH (10 mL). To this solution, acetone (2.94 mL, 39.9 mmol) and acetic acid (0.011 mL, 0.200 mmol) were added at 0° C., and the reaction was stirred at RT for 1 h. To this reaction mixture, sodium cyanoborohydride (0.753 g, 11.98 mmol) was added at 0° C., and the reaction was stirred at RT for 16 h. After completion, the reaction was concentrated under reduced pressure. The residue was quenched with saturated sodium bicarbonate solution (25 mL) and the mixture was extracted with 3% MeOH in DCM (3×25 mL). The combined organic layer was washed with brine solution (25 mL), dried over anhydrous sodium sulfate, and filtered, and the filtrate was concentrated under reduced pressure to obtain crude tert-butyl ((1s,3s)-3-(isopropylamino)-3-methylcyclobutyl)carbamate (0.968 g, 87% yield) as a semi-solid. This material was used without further purification. ¹H NMR (400 MHz, DMSO-d₆): δ 6.97 (br d, J=6.5 Hz, 1H), 3.77-3.67 (m, 1H), 3.03-2.90 (m, 1H), 2.20-2.12 (m, 2H), 1.90-1.79 (m, 2H), 1.33 (s, 9H), 1.19 (s, 3H), 0.98 (d, J=6.3 Hz, 6H), one proton merged with solvent peaks.

### Step 2. tert-Butyl 4-((2-(5-(2-(((1s,3s)-3-((tert-butoxycarbonyl)amino)-1-methylcyclobutyl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate

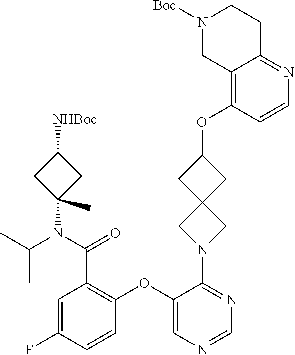 [SMILES: CC(C)N(C(=O)c1cc(F)ccc1Oc1cncnc1N1CC2(CC(Oc3ccnc4c3CN(C(=O)OC(C)(C)C)CC4)C2)C1)[C@]1(C)C[C@H](NC(=O)OC(C)(C)C)C1]

This compound was synthesized following the procedure described for the synthesis of Example 20, step 2, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid and 2 equivalents of tert-butyl ((1s,3s)-3-(isopropylamino)-3-methylcyclobutyl)carbamate, except that 25 equivalents of DIPEA was used. After work-up, the crude was purified by reverse phase chromatography using a SiliCycle SiliaSep C18 column (40 g) on Biotage isolera one (Mobile phase A: 10 mM NH₄HCO₃ in water, B: ACN) to obtain tert-butyl 4-((2-(5-(2-(((1s,3s)-3-((tert-butoxycarbonyl)amino)-1-methylcyclobutyl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate as a semi-solid.

Yield: 27.5%; LCMS (Method A): Rt=2.22 min, m/z=802.8 [M+H]⁺, 63.54%.

### Step 3. N-((1s,3s)-3-Amino-1-methylcyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

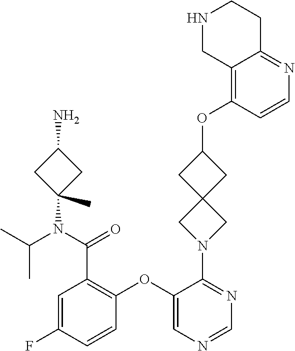 [SMILES: CC(C)N(C(=O)c1cc(F)ccc1Oc1cncnc1N1CC2(CC(Oc3ccnc4c3CNCC4)C2)C1)[C@]1(C)C[C@H](N)C1]

This compound was synthesized following the procedure described for the synthesis of Example 20, step 3, starting with 1 equivalent of tert-butyl 4-((2-(5-(2-(((1s,3s)-3-((tert-butoxycarbonyl)amino)-1-methylcyclobutyl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate, except that 1 equivalent of TMSCl was used. After completion, the reaction was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain N-((1s,3s)-3-amino-1-methylcyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 54.2%; ¹H NMR (400 MHz, DMSO-d₆): δ 8.29 (s, 1H), 8.16 (d, J=5.5 Hz, 1H), 7.73-7.66 (m, 1H), 7.28-7.15 (m, 2H), 7.09-6.92 (m, 1H), 6.68-6.58 (m, 1H), 4.77-4.67 (m, 1H), 4.40-4.08 (m, 4H), 3.69 (s, 2H), 3.12-3.00 (m, 1H), 2.98-2.89 (m, 3H), 2.64-274 (m, 2H), 2.72-2.67 (m, 2H), 2.29-2.20 (m, 2H), 2.14-2.01 (m, 2H), 2.00-1.78 (m, 2H), 1.49-1.12 (m, 9H), three protons merged in solvent peaks; LCMS (Method A): Rt=1.42 min, m/z=602.2 [M+H]⁺, 75.97%.

### Step 4. N-((1s,3s)-3-(Dimethylamino)-1-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

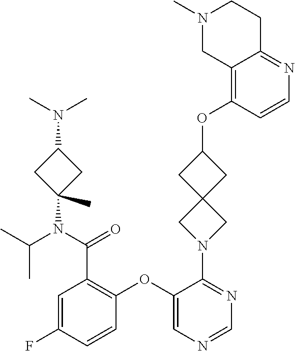

This compound was synthesized following the procedure described for the synthesis of Example 20, step 4, starting with 1 equivalent of N-((1s,3s)-3-amino-1-methylcyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide, except that 75 equivalents of formaldehyde (37% in H₂O), 0.1 equivalent of AcOH, and 4 equivalents of STAB were used. After completion, the reaction mixture was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain N-((1s,3s)-3-(dimethylamino)-1-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 68.4%; ¹H NMR (400 MHz, DMSO-d₆): δ 8.29-8.22 (m, 1H), 8.19 (d, J=5.3 Hz, 1H), 7.67-7.56 (m, 1H), 7.35-7.20 (m, 2H), 7.19-7.01 (m, 1H), 6.71-6.58 (m, 1H), 4.81-4.70 (m, 1H), 4.41-4.11 (m, 4H), 3.79-3.64 (m, 1H), 2.84-2.74 (m, 4H), 2.62 (t, J=5.8 Hz, 2H), 2.36 (s, 3H), 2.31-2.22 (m, 2H), 2.20-2.08 (m, 2H), 2.05-1.87 (m, 8H), 1.50-1.12 (m, 9H), three protons merged with solvent peaks; LCMS (Method A): Rt=1.71 min, m/z=644.2 [M+H]⁺; HPLC (Method A): Rt=4.98 min, 99.82%; Chiral SFC (Method C): Rt=2.72 min, 99.72%.

### Example 43. N-((1r,3r)-3-(Dimethylamino)-1-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 43)

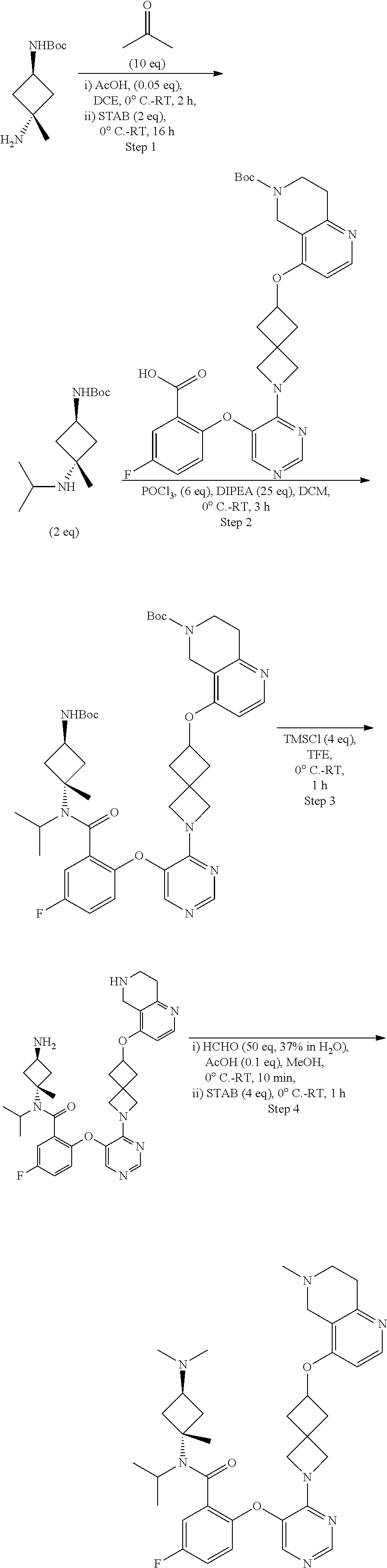

### Step 1. tert-Butyl ((1r,3r)-3-(isopropylamino)-3-methylcyclobutyl)carbamate

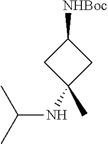 [SMILES: CC(C)N[C@]1(C)C[C@@H](NC(=O)OC(C)(C)C)C1]

This compound was synthesized following the procedure described for the synthesis of Example 20, step 1, starting with 1 equivalent of tert-butyl ((1r,3r)-3-amino-3-methylcyclobutyl)carbamate, except that 10 equivalents of acetone, 0.05 equivalent of AcOH, and 2 equivalents of STAB were used, and DCE was used as the solvent. After completion, the reaction was concentrated under reduced pressure to obtain crude tert-butyl ((1r,3r)-3-(isopropylamino)-3-methylcyclobutyl)carbamate as a semi-solid. This material was used without further purification.

Yield: 65.5%, ¹H NMR (400 MHz, DMSO-d₆): δ 7.03 (br s, J=7.4 Hz, 1H), 4.01-3.84 (m, 1H), 2.84-2.73 (m, 1H), 2.16-2.03 (m, 2H), 1.76-1.67 (m, 2H), 1.37 (s, 9H), 1.17 (s, 3H), 0.96 (d, J=6.3 Hz, 6H), one proton merged with solvent peaks.

### Step 2. tert-Butyl 4-((2-(5-(2-((1r,3r)-3-((tert-butoxycarbonyl)amino)-1-methylcyclobutyl)(isopropyl)carbamoyl)-4-fluorophenoxy)pyrimidin-4-yl)-2-azaspiro[3.3]heptan-6-yl)oxy)-7,8-dihydro-1,6-naphthyridine-6(SH)-carboxylate

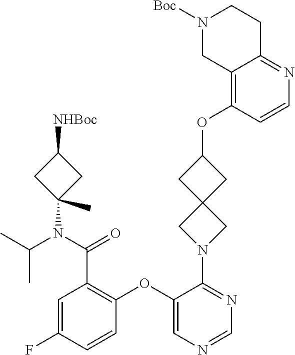 [SMILES: CC(C)N(C(=O)c1cc(F)ccc1Oc1cncnc1N1CC2(CC(Oc3ccnc4c3CN(C(=O)OC(C)(C)C)CC4)C2)C1)[C@]1(C)C[C@@H](NC(=O)OC(C)(C)C)C1]

This compound was synthesized following the procedure described for the synthesis of Example 20, step 1, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid and 2 equivalents of tert-butyl ((1r,3r)-3-(isopropylamino)-3-methylcyclobutyl)carbamate, except that 25 equivalents of DIPEA was used. After work-up, the crude was purified by reverse phase chromatography using a SiliCycle SiliaSep C18 column (40 g) on Biotage isolera one (Mobile phase A: 10 mM NH₄HCO₃ in water, B: ACN) to obtain tert-butyl 4-((2-(5-(2-(((1r,3r)-3-((tert-butoxycarbonyl)amino)-1-methylcyclobutyl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate as a solid.

Yield: 18.61%; LCMS (Method A): Rt=2.22 min, m/z=802.3 [M+H]⁺, 86.12%

### Step 3. N-((1r,3r)-3-Amino-1-methylcyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

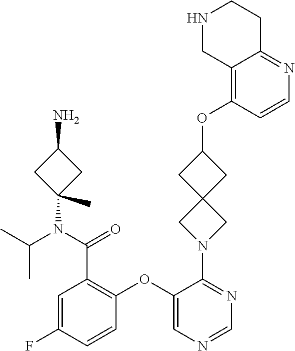 [SMILES: CC(C)N(C(=O)c1cc(F)ccc1Oc1cncnc1N1CC2(CC(Oc3ccnc4c3CNCC4)C2)C1)[C@]1(C)C[C@@H](N)C1]

This compound was synthesized following the procedure described for the synthesis of Example 20, step 3, starting with 1 equivalent of tert-butyl 4-((2-(5-(2-(((1r,3r)-3-((tert-butoxycarbonyl)amino)-1-methylcyclobutyl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain N-((1r,3r)-3-amino-1-methylcyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 61.2%; LCMS (Method H): Rt=0.99 min, m/z=602.3 [M+H]⁺, 98.11%.

### Step 4. N-((1r,3r)-3-(Dimethylamino)-1-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

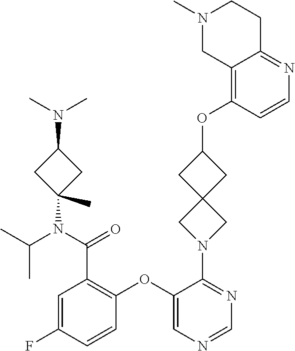 [SMILES: CC(C)N(C(=O)c1cc(F)ccc1Oc1cncnc1N1CC2(CC(Oc3ccnc4c3CN(C)CC4)C2)C1)[C@]1(C)C[C@@H](N(C)C)C1]

This compound was synthesized following the procedure described for the synthesis of Example 20, step 4, starting with 1 equivalent of N-((1r,3r)-3-amino-1-methylcyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide, except that, 50 equivalents of formaldehyde (37% in H2O), 0.1 equivalent of AcOH, and 4 equivalents of STAB were used. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain N-((1r,3r)-3-(dimethylamino)-1-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 49.7%; ¹H NMR (400 MHz, DMSO-d₆): δ 8.28 (s, 1H), 8.18 (d, J=5.6 Hz, 1H), 7.67 (s, 1H), 7.34-7.19 (m, 2H), 7.11-6.99 (m, 1H), 6.66 (d, J=5.6 Hz, 1H), 4.80-4.69 (m, 1H), 4.37-4.12 (m, 4H), 3.83-3.69 (m, 1H), 3.36 (s, 2H), 2.86-2.75 (m, 4H), 2.63 (t, J=5.8 Hz, 2H), 2.36 (s, 3H), 2.32-2.18 (m, 3H), 2.09-1.90 (m, 8H), 1.62-1.13 (m, 9H), two protons merged with solvent peaks; LCMS (Method C): Rt=1.35 min, m/z=644.3 [M+H]⁺; HPLC (Method A): Rt=4.99 min, 99.79%; Chiral SFC (Method AI): Rt=2.86 min, 100%.

### Example 44. N-(3-(Dimethylamino)propyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 44)

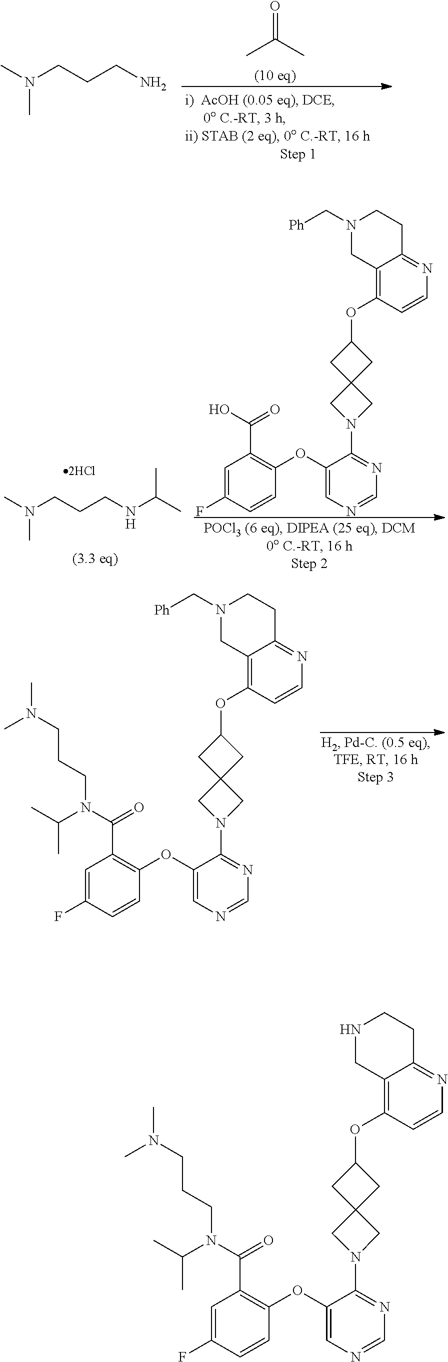

### Step 1. N¹-Isopropyl-N³,N³-dimethylpropane-1,3-diamine dihydrochloride dihydrochloride

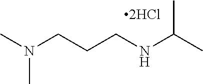

In a dried, 100 mL two neck round bottom flask under a nitrogen atmosphere, N¹,N¹-dimethylpropane-1,3-diamine (2 g, 19.57 mmol) was dissolved in DCE (15 mL). To this solution, acetone (14.39 mL, 196 mmol) and acetic acid (0.056 mL, 0.979 mmol) were added at 0° C. and the reaction was stirred at RT for 3 h. To this reaction mixture, STAB (8.30 g, 39.1 mmol) was added at 0° C., and the reaction was stirred at RT for 16 h. The reaction progress was monitored by LCMS. After completion, the reaction was filtered through a Celite® pad, and the pad was washed 5 with DCM (2×25 mL). The filtrate was cooled to 0° C., 4 M HCl in 1,4-dioxane (3 mL) was added, and the reaction was stirred at RT for 15 min. The solvent was evaporated under reduced pressure, and the residue was triturated with EtOAc (2×20 mL). The solvent was decanted, and the solid was dried under vacuum to obtain crude N¹-isopropyl-N,N-dimethylpropane-1,3-diamine dihydrochloride (1.4 g, 39.6% yield) as a solid. This material was used without further purification.

¹H NMR (400 MHz, DMSO-d₆): δ 11.71-8.71 (m, 3H), 3.28-3.21 (m, 1H), 3.21-3.10 (m, 2H), 3.03-2.95 (m, 2H), 2.75-2.69 (m, 6H), 2.21-2.06 (m, 2H), 1.26 (d, J=6.5 Hz, 6H).

### Step 2. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-N-(3-(dimethylamino)propyl)-5-fluoro-N-isopropylbenzamide

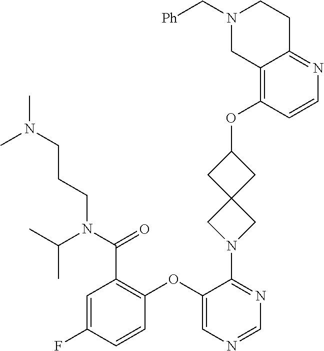 [SMILES: CC(C)N(CCCN(C)C)C(=O)c1cc(F)ccc1Oc1cncnc1N1CC2(CC(Oc3ccnc4c3CN(Cc3ccccc3)CC4)C2)C1]

This compound was synthesized following the procedure described for the synthesis of Example 20, step 2, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 3.3 equivalents of N¹-Isopropyl-N³,N³-dimethylpropane-1,3-diamine dihydrochloride, except that 25 equivalents of DIPEA was used. After completion, the reaction was quenched with aq. NaHCO₃ solution and the mixture was extracted with 5% MeOH in DCM. The combined organic layer was dried over anhydrous sodium sulfate and filtered, and the filtrate was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-(3-(dimethylamino)propyl)-5-fluoro-N-isopropylbenzamide as a solid.

Yield: 32.5%; LCMS (Method A): Rt=1.99 min, m/z=694.4 [M+H]⁺, 86.40%.

### Step 3. N-(3-(Dimethylamino)propyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

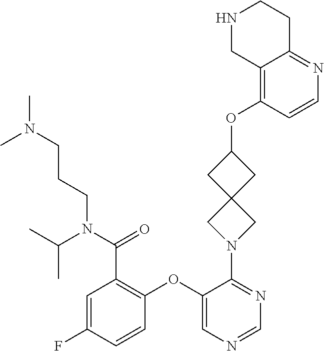 [SMILES: CC(C)N(CCCN(C)C)C(=O)c1cc(F)ccc1Oc1cncnc1N1CC2(CC(Oc3ccnc4c3CNCC4)C2)C1]

This compound was synthesized following the procedure described for the synthesis of Example 20, step 5, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-(3-(dimethylamino)propyl)-5-fluoro-N-isopropylbenzamide, except that 0.5 equivalent of Pd-C was used. After filtration and concentration, the crude was purified by reverse phase chromatography using a SiliCycle SiliaSep C18 column (80 g) on Biotage isolera one (Mobile phase A: 10 mM NH₄HCO₃ in water, B: ACN) to obtain N-(3-(dimethylamino)propyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 54.9%; ¹H NMR (400 MHz, DMSO-d₆): δ 8.29-8.28 (m, 1H), 8.16 (d, J=5.6 Hz, 1H), 7.75-7.67 (m, 1H), 7.35-7.22 (m, 2H), 7.05 (dd, J=4.3, 9.1 Hz, 1H), 6.64 (d, J=5.6 Hz, 1H), 4.73 (quin, J=6.5 Hz, 1H), 4.32-4.09 (m, 4H), 3.80-3.76 (m, 1H), 3.69 (s, 2H), 3.20-3.09 (m, 2H), 2.94 (t, J=5.8 Hz, 2H), 2.82-2.73 (m, 2H), 2.70-2.67 (m, 2H), 2.28-2.18 (m, 4H), 2.13-1.88 (m, 7H), 1.80-1.49 (m, 2H), 1.12-1.00 (m, 6H); LCMS (Method H): Rt=0.99 min, m/z=604.3 [M+H]⁺; HPLC (Method A): Rt=3.99 min, 99.61%.

### Example 45. N-(2-(Dimethylamino)ethyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 45)

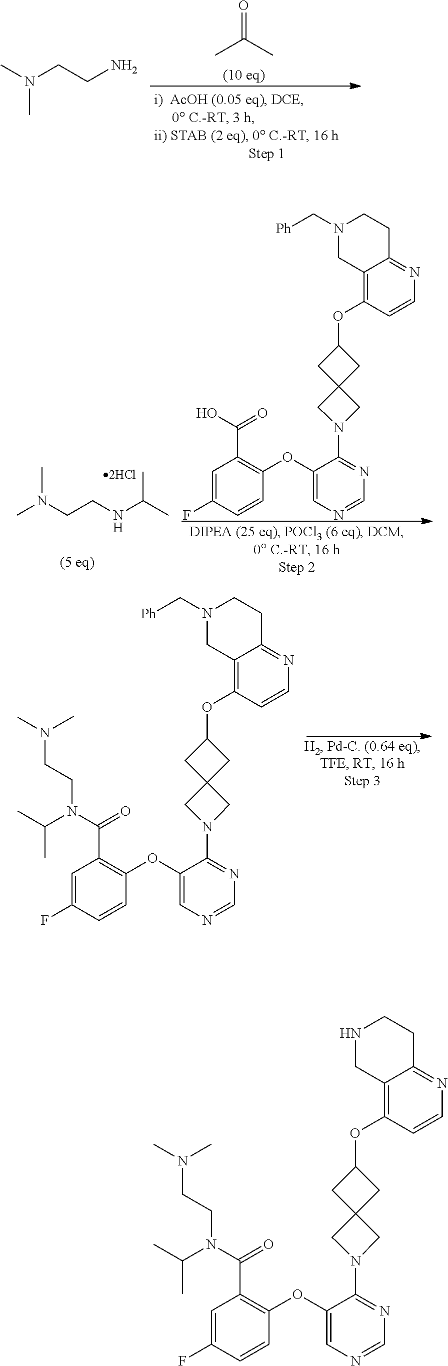

### Step 1. N¹—Isopropyl-N²,N²-dimethylethane-1,2-diamine dihydrochloride

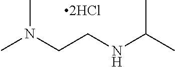

This compound was synthesized following the procedure described for the synthesis of Example 44, Step 1, starting with 1 equivalent of N¹,N¹-dimethylethane-1,2-diamine and 10 equivalents of acetone. After work-up, the solvent was decanted and the solid was dried under vacuum to obtain crude N¹-isopropyl-N³,N³-dimethylethane-1,2-diamine dihydrochloride as a solid. This material was used without further purification.

Yield: 58.2%; ¹H NMR (400 MHz, CDCl₃): δ 10.34 (br s, 3H), 3.33-3.24 (m, 1H), 3.13-3.06 (m, 2H), 2.92-2.86 (m, 2H), 2.42 (s, 6H), 1.32 (d, J=6.5 Hz, 6H).

### Step 2. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-N-(2-(dimethylamino)ethyl)-5-fluoro-N-isopropylbenzamide

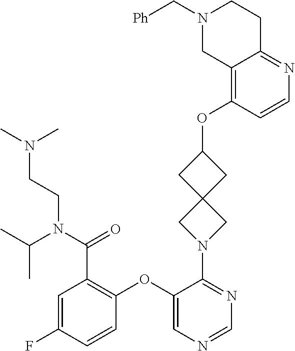 [SMILES: CC(C)N(CCN(C)C)C(=O)c1cc(F)ccc1Oc1cncnc1N1CC2(CC(Oc3ccnc4c3CN(Cc3ccccc3)CC4)C2)C1]

This compound was synthesized following the procedure described for the synthesis of Example 20, step 2, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 5 equivalents of N¹-Isopropyl-N³,N³-dimethylethane-1,2-diamine dihydrochloride, except that 25 equivalents of DIPEA was used. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-(2-(dimethylamino)ethyl)-5-fluoro-N-isopropylbenzamide as a solid.

Yield: 17.28%, LCMS (Method A): Rt=1.86 min, m/z=680.3 [M+H]⁺, 99.24%.

### Step 3. N-(2-(Dimethylamino)ethyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

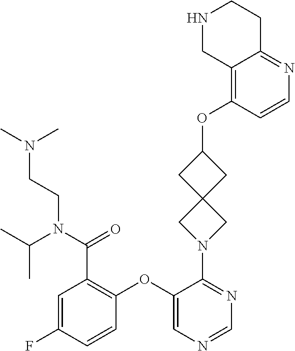 [SMILES: CC(C)N(CCN(C)C)C(=O)c1cc(F)ccc1Oc1cncnc1N1CC2(CC(Oc3ccnc4c3CNCC4)C2)C1]

This compound was synthesized following the procedure described for the synthesis of Example 20, step 5, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-(2-(dimethylamino)ethyl)-5-fluoro-N-isopropylbenzamide, except that 0.5 equivalent of Pd-C was used. After filtration and concentration, the crude was purified by reverse phase chromatography using a SiliCycle SiliaSep C18 column (80 g) on Biotage isolera one (Mobile phase A: 10 mM NH₄HCO₃ in water, B: ACN) to obtain N-(2-(dimethylamino)ethyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 62.9%; ¹H NMR (400 MHz, DMSO-d₆): δ 8.30-8.27 (m, 1H), 8.17-8.14 (m, 1H), 7.75-7.62 (m, 11H), 7.36-7.22 (m, 2H), 7.11-7.02 (m, 1H), 6.64 (d, J=5.6 Hz, 1H), 4.73 (quin, J=6.5 Hz, 1H), 4.43-4.08 (m, 4H), 3.79-3.71 (mn, 1H), 3.69 (s, 2H), 3.49-3.37 (m, 1H), 3.28-3.11 (mn, 2H), 2.94 (t, J=5.7 Hz, 2H), 2.83-2.74 (mn, 2H), 2.67 (br t, J=5.1 Hz, 2H), 2.46-2.40 (m, 1H), 2.31-2.21 (m, 3H), 2.19-1.92 (m, 6H), 1.21-1.00 (m, 6H); LCMS (Method H): Rt=0.96 min, m/z=590.4 [M+H]⁺; HPLC (Method A); Rt=4.12 min, 99.73%.

### Example 46. N-((1s,4s)-4-(Dimethylamino)cyclohexyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 46)

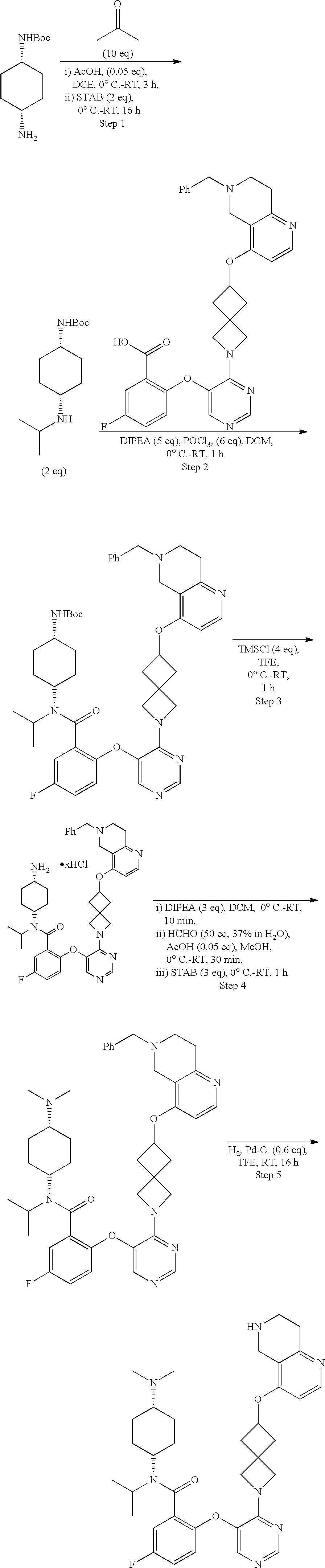

### Step 1. tert-Butyl ((1s,4s)-4-(isopropylamino)cyclohexyl)carbamate

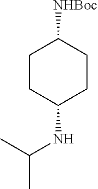 [SMILES: CC(C)N[C@H]1CC[C@@H](NC(=O)OC(C)(C)C)CC1]

This compound was synthesized following the procedure described for the synthesis of Example 20, step 1, starting with 1 equivalent of tert-butyl ((1s,4s)-4-aminocyclohexyl)carbamate, except that 10 equivalents of acetone, 0.05 equivalent of AcOH, 2 equivalents of STAB were used, and DCE was used as the solvent. After work-up, the filtrate was concentrated under reduced pressure to obtain crude tert-butyl ((1s,4s)-4-(isopropylamino)cyclohexyl)carbamate as a semi-solid. This material was used without further purification.

Yield: 74.9%; ¹H NMR (400 MHz, CDCl₃): δ 4.75 (br s, 1H), 3.74-3.65 (m, 1H), 3.03-2.92 (m, 1H), 2.77-2.65 (m, 1H), 1.76-1.56 (m, 6H), 1.45 (s, 9H), 1.42-1.35 (m, 2H), 1.08 (d, J=6.3 Hz, 6H), one proton merged with solvent peaks.

### Step 2. tert-Butyl ((1s,4s)-4-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclohexyl)carbamate

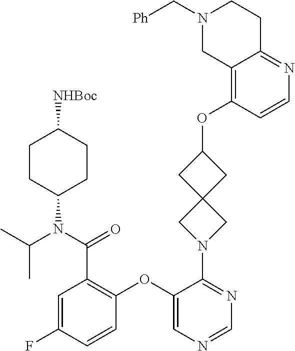 [SMILES: CC(C)N(C(=O)c1cc(F)ccc1Oc1cncnc1N1CC2(CC(Oc3ccnc4c3CN(Cc3ccccc3)CC4)C2)C1)[C@H]1CC[C@@H](NC(=O)OC(C)(C)C)CC1]

This compound was synthesized following the procedure described for the synthesis of Example 20, step 2, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 2 equivalents of tert-butyl ((1s,4s)-4-(isopropylamino)cyclohexyl)carbamate, except that 5 equivalents of DIPEA was used. After work-up, the crude compound was purified by reverse phase chromatography using a SiliCycle SiliaSep C18 column (80 g) on Biotage isolera one (Mobile phase A: 10 mM NH₄HCO₃ in water, B: ACN) to obtain tert-butyl ((1s,4s)-4-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclohexyl)carbamate as a solid.

Yield: 6.69%; LCMS (Method A): Rt=2.34 min, m/z=806.4 [M+H]⁺, 83.39%.

### Step 3. N-((1s,4s)-4-Aminocyclohexyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl

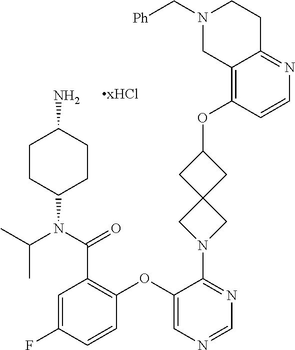

This compound was synthesized following the procedure described for the synthesis of Example 20, step 3, starting with 1 equivalent of tert-butyl ((1s,4s)-4-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclohexyl)carbamate. After completion, the reaction was concentrated under reduced pressure to obtain crude N-((1s,4s)-4-aminocyclohexyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl as a solid. This material was used without further purification.

Yield: 22.88%; LCMS (Method A): Rt=2.12 min, m/z=706.2 [M+H]⁺, 23.12%.

### Step 4. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-N-((1s,4s)-4-(dimethylamino)cyclohexyl)-5-fluoro-N-isopropylbenzamide

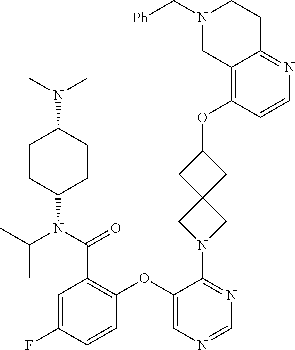

This compound was synthesized following the procedure described for the synthesis of Example 20, step 4, starting with 1 equivalent of N-((1s,4s)-4-aminocyclohexyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide, except that 3 equivalents of DIPEA, 0.05 equivalent of AcOH, 50 equivalents of formaldehyde (37% in H₂O), and 3 equivalents of STAB were used. After completion, the reaction was concentrated under reduced pressure. The crude was purified by Prep HPLC (Method A) to obtain 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1s,4s)-4-(dimethylamino)cyclohexyl)-5-fluoro-N-isopropylbenzamide as a solid.

Yield: 10.70%; LCMS (Method A): Rt=2.26 min, m/z=734.3 [M+H]⁺, 95.34%.

### Step 5. N-((1s,4s)-4-(Dimethylamino)cyclohexyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

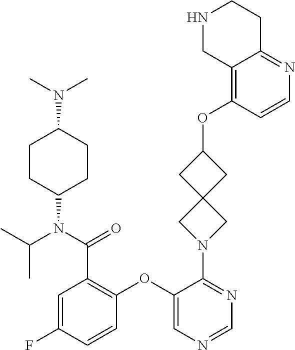

This compound was synthesized following the procedure described for the synthesis of Example 20, step 5, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1s,4s)-4-(dimethylamino)cyclohexyl)-5-fluoro-N-isopropylbenzamide xHCl, except that 0.6 equivalent of Pd-C was used. After filtration and concentration, the crude was purified by reverse phase chromatography using a SiliCycle SiliaSep C18 column (80 g) on Biotage isolera one (Mobile phase A: 10 mM NH₄HCO₃ in water, B: ACN) to obtain N-((1s,4s)-4-(dimethylamino)cyclohexyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 48.7%; ¹H NMR (400 MHz, DMSO-d₆): δ 8.26 (s, 1H), 8.16 (d, J=5.6 Hz, 1H), 7.72-7.66 (m, 1H), 7.29-7.19 (m, 2H), 7.14-7.07 (m, 1H), 6.63 (d, J=5.8 Hz, 1H), 4.78-4.69 (m, 1H), 4.34-4.09 (m, 4H), 3.70 (s, 2H), 3.47-3.40 (m, 1H), 3.28-3.20 (m, 1H), 2.94 (t, J=5.8 Hz, 2H), 2.83-2.72 (m, 2H), 2.71-2.67 (m, 2H), 2.23-2.18 (m, 2H), 2.14-2.06 (m, 6H), 2.01-1.84 (m, 2H), 1.83-1.69 (m, 3H), 1.47-0.91 (m, 10H), one proton merged with solvent peaks; LCMS (Method C): Rt=1.51 min, m/z=644.3 [M+H]⁺; HPLC (Method A): Rt=4.47 min, 99.04%; Chiral SFC (Method A). Rt=2.68 min, 97.42%.

### Example 47. N-((1r,4r)-4-(Dimethylamino)cyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 47)

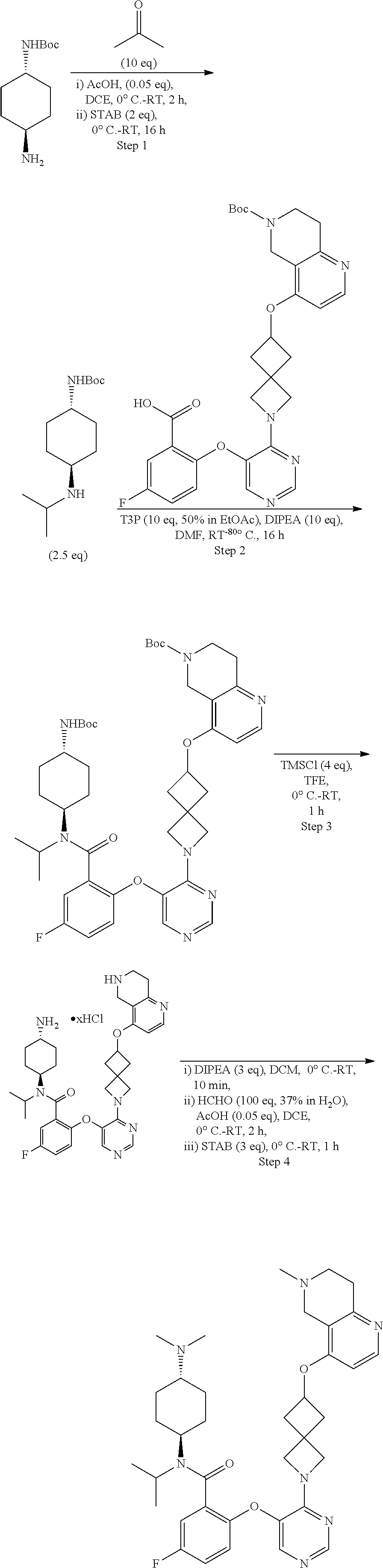

### Step 1. tert-Butyl ((1r,4r)-4-(isopropylamino)cyclohexyl)carbamate

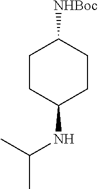 [SMILES: CC(C)N[C@H]1CC[C@H](NC(=O)OC(C)(C)C)CC1]

This compound was synthesized following the procedure described for the synthesis of Example 20, step 1, starting with 1 equivalent of tert-butyl ((1r,4r)-4-aminocyclohexyl)carbamate, except that 10 equivalents of acetone, 0.05 equivalents of ACOH, 2 equivalents of STAB were used, and DCE was used as the solvent. After work-up, the filtrate was concentrated under reduced pressure to obtain crude tert-butyl ((1r,4r)-4-(isopropylamino)cyclohexyl)carbamate. This material was used without further purification.

Yield: 79%, ¹H NMR (400 MHz, CDCl₃): δ 4.38 (br s, 1H), 3.50-3.36 (m, 1H), 3.04-2.91 (m, 1H), 2.56-2.48 (m, 1H), 2.07-2.00 (m, 2H), 1.99-1.92 (m, 2H), 1.46 (s, 9H), 1.20-1.11 (m, 4H), 1.06 (d, J=6.3 Hz, 6H), one proton merged with solvent peaks.

### Step 2. tert-Butyl 4-((2-(5-(2-(((1r,4r)-4-((tert-butoxycarbonyl)amino)cyclohexyl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate

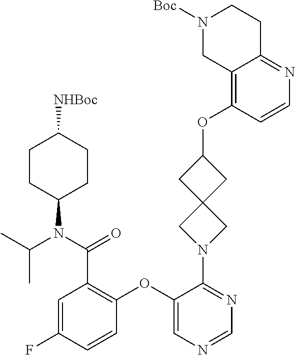 [SMILES: CC(C)N(C(=O)c1cc(F)ccc1Oc1cncnc1N1CC2(CC(Oc3ccnc4c3CN(C(=O)OC(C)(C)C)CC4)C2)C1)[C@H]1CC[C@H](NC(=O)OC(C)(C)C)CC1]

This compound was synthesized following the procedure described for the synthesis of Example 25, step 1, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid and 2.5 equivalents of tert-butyl ((1r,4r)-4-(isopropylamino)cyclohexyl)carbamate, except that 10 equivalents of T3P (50% in EtOAc) and 10 equivalents of DIPEA were used. The crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl 4-((2-(5-(2-(((1r,4r)-4-((tert-butoxycarbonyl)amino)cyclohexyl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate as a solid.

Yield: 47.9%; LCMS (Method A): Rt=2.26 min, m/z=816.2 [M+H]⁺, 90.29%

### Step 3. N-((1r,4r)-4-Aminocyclohexyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl

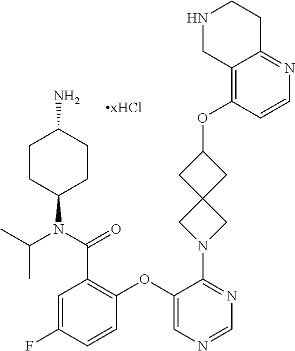 [SMILES: CC(C)N(C(=O)c1cc(F)ccc1Oc1cncnc1N1CC2(CC(Oc3ccnc4c3CNCC4)C2)C1)[C@H]1CC[C@H](N)CC1]

This compound was synthesized following the procedure described for the synthesis of Example 20, step 3, starting with 1 equivalent of tert-butyl 4-((2-(5-(2-(((1r,4r)-4-((tert-butoxycarbonyl)amino)cyclohexyl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate. After completion, the crude was triturated with EtOAc, and the supernatant was decanted. The residue was dried under reduced pressure to obtain crude N-((1r,4r)-4-aminocyclohexyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl. This material was used without further purification.

Yield: 91%, LCMS (Method A): Rt=1.36 min, m/z=616.2 [M+H]⁺, 76.23%.

### Step 4. N-((1r,4r)-4-(Dimethylamino)cyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

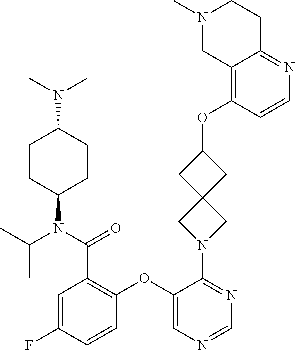 [SMILES: CC(C)N(C(=O)c1cc(F)ccc1Oc1cncnc1N1CC2(CC(Oc3ccnc4c3CN(C)CC4)C2)C1)[C@H]1CC[C@H](N(C)C)CC1]

This compound was synthesized following the procedure described for the synthesis of Example 20, step 4, starting with 1 equivalent of N-((1r,4r)-4-aminocyclohexyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl, except that 3 equivalents of DIPEA, 0.05 equivalent of AcOH, 100 equivalents of formaldehyde (37% in H₂O), and 3 equivalents of STAB were used. After completion, the crude was purified by reverse phase chromatography using a SiliCycle SiliaSep C18 column (80 g) on Biotage isolera one (Mobile phase A: 10 mM NH₄HCO₃ in water, B: ACN) to obtain N-((1r,4r)-4-(dimethylamino)cyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 49.4%; ¹H NMR (400 MHz, DMSO-dc): 6 8.27 (d, J=3.9 Hz, 1H), 8.18 (d, J=5.5 Hz, 1H), 7.70 (d, J=6.6 Hz, 1H), 7.29-7.20 (m, 2H), 7.11-7.07 (m, 1H), 6.66 (d, J=5.6 Hz, 1H), 4.80-4.71 (m, 1H), 4.35-4.09 (m, 4H), 3.74-3.50 (m, 1H), 3.37 (s, 2H), 3.20-2.98 (m, 1H), 2.85-2.74 (m, 4H), 2.63 (t, J=5.8 Hz, 2H), 2.37 (s, 3H), 2.30-2.20 (m, 2H), 2.15 (s, 3H), 2.07 (s, 3H), 1.85-1.50 (m, 4H), 1.49-1.20 (m, 6H), 1.13-0.90 (m, 4H), one proton merged with solvent peaks; LCMS (Method H): Rt=1.60 min, m/z=658.5 [M+H]⁺; HPLC (Method A): Rt=4.57 min, 98.44%; Chiral SFC (Method AF): Rt=5.10 min, 100%.

### Example 48. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(1-methylpiperidin-4-yl)benzamide (Compound No. 48)

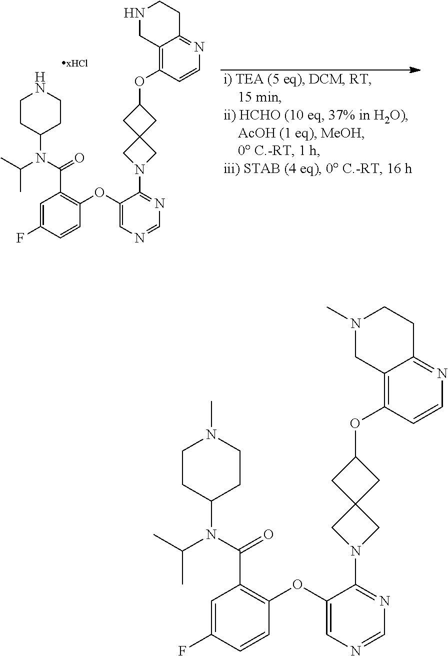 [SMILES: CC(C)N(C(=O)c1cc(F)ccc1Oc1cncnc1N1CC2(CC(Oc3ccnc4c3CNCC4)C2)C1)C1CCNCC1 ; CC(C)N(C(=O)c1cc(F)ccc1Oc1cncnc1N1CC2(CC(Oc3ccnc4c3CN(C)CC4)C2)C1)C1CCN(C)CC1]

This compound was synthesized following the procedure described for the synthesis of Example 20, step 4, starting with 1 equivalent of 5-fluoro-N-isopropyl-N-(piperidin-4-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl, except that 5 equivalents of TEA, 1 equivalent of AcOH and 4 equivalents of STAB were used. After work-up, the crude compound was purified twice by reverse phase chromatography using a SiliCycle SiliaSep C18 column (60 g) on Biotage isolera one (Mobile phase A: 10 mM NH₄HCO₃ in water, B: ACN). The pure fractions were concentrated under reduced pressure and lyophilized to afford 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(1-methylpiperidin-4-yl)benzamide as a solid. Yield: 13.01%; ¹H NMR (400 MHz, DMSO-d₆): δ 8.30-8.24 (m, 1H), 8.18 (d, 0.1=5.6 Hz, 1H), 7.76-7.62 (m, 1H), 7.30-7.21 (m, 2H), 7.14-7.03 (m, 1H), 6.66 (d, J=5.8 Hz, 1H), 4.75 (quin, J=6.5 Hz, 1H), 4.32-4.17 (m, 3H), 4.16-4.09 (m, 1H), 3.77-3.66 (m, 1H), 3.58-3.46 (m, 1H), 3.37 (s, 2H), 3.16-2.95 (m, 11H), 2.85-2.69 (m, 7H), 2.66-2.61 (m, 2H), 2.36 (s, 3H), 2.28-2.20 (m, 2H), 2.16-2.02 (m, 3H), 1.98-1.87 (m, 1H), 1.76-1.55 (m, 2H), 1.47-0.95 (m, 6H), one proton merged with solvent peaks; LCMS (Method H): Rt=0.93 min, m/z=630.4 [M+H]⁺; HPLC (Method A): Rt=4.61 min, 99.79%.

### Example 49. (R)-5-Fluoro-N-isopropyl-N-(1-methylpiperidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 49)

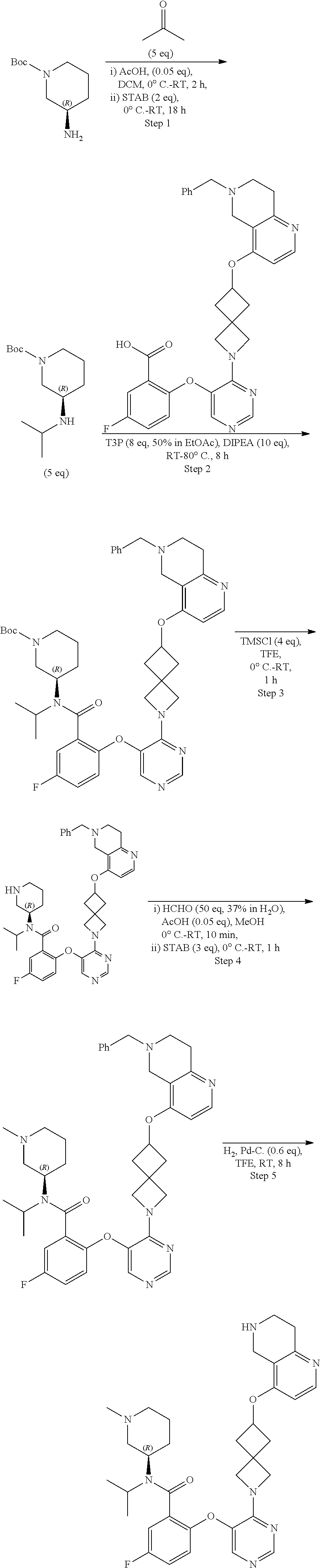

### Step 1. tert-Butyl (R)-3-(isopropylamino)piperidine-1-carboxylate

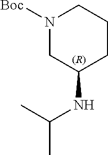

This compound was synthesized following the procedure described for the synthesis of Intermediate 1, starting with 1 equivalent of tert-butyl (R)-3-aminopiperidine-1-carboxylate, except that 5 equivalents of acetone, and 0.5 equivalent of AcOH were used. After completion, the reaction was quenched with saturated sodium bicarbonate solution and extracted with DCM. The combined organic layer was dried over sodium sulfate and filtered. The filtrate was concentrated under reduced pressure to obtain crude tert-butyl (R)-3-(isopropylamino)piperidine-1-carboxylate. This material was used without further purification.

Yield: 91%; ¹H NMR (400 MHz, CDCl₃): δ 4.12-3.85 (m, 1H), 3.79-3.76 (m, 1H), 3.04-2.93 (m, 1H), 2.93-2.81 (m, 1H), 2.74-2.56 (m, 2H), 1.95-1.86 (m, 1H), 1.72-1.60 (m, 2H), 1.47 (s, 9H), 1.34-1.21 (m, 1H), 1.07 (d, J=6.3 Hz, 6H), one proton merged with solvent peaks; LCMS (Method B): Rt=1.03 min, m/z=243.3 [M+H]⁺, 88.07%.

### Step 2. tert-Butyl (R)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)piperidine-1-carboxylate

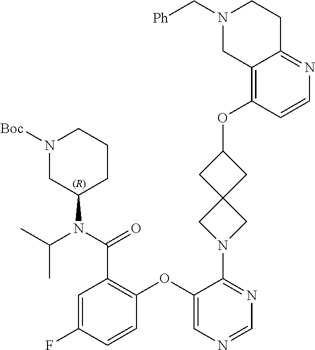

This compound was synthesized following the procedure described for the synthesis of Example 25, step 1, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 5 equivalents of tert-butyl (R)-3-(isopropylamino)piperidine-1-carboxylate, except that 8 equivalents of T3P (50% in EtOAc) and 10 equivalents of DIPEA were used. After completion, the crude was purified by reverse phase chromatography using a SiliCycle SiliaSep C18 column (80 g) on Biotage isolera one (Mobile phase A: 10 mM NHaHCO₃ in water, B: ACN) to obtain tert-butyl (R)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)piperidine-1-carboxylate as a solid.

Yield: 57.3%; LCMS (Method A): Rt=2.36 min, m/z=792.2 [M+H]⁺, 83.23%.

### Step 3. (R)-2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(piperidin-3-yl)benzamide

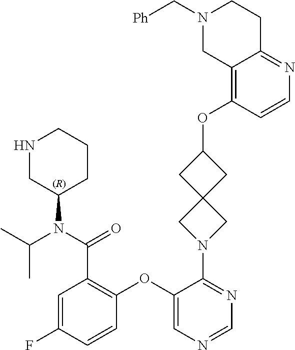

This compound was synthesized following the procedure described for the synthesis of Example 20, step 3, starting with 1 equivalent of tert-butyl (R)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)piperidine-1-carboxylate. After completion, the reaction was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain (R)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(piperidin-3-yl)benzamide as a solid.

Yield: 71.8%; LCMS (Method A): Rt=1.80 min, m/z=692.2 [M+H]⁺, 94.12%.

### Step 4. (R)-2-((4-(6-((6-Benzyl-5,6,7,8-tetrahydro-,6-naphthyridin-4-yl)oxy)-2-azaspiro[3.3]heptan-2-yl)pyrimidin-5-yl)oxy)-5-fluoro-N-isopropyl-N-(1-methylpiperidin-3-yl)benzamide

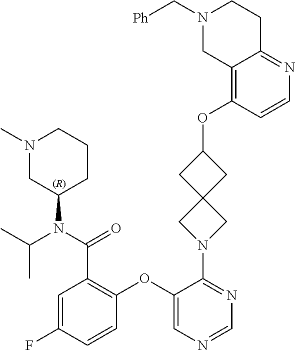

This compound was synthesized following the procedure described for the synthesis of Example 20, Step 4, starting with 1 equivalent of (R)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(piperidin-3-yl)benzamide, except that 50 equivalents of formaldehyde (37% in H₂O), 0.05 equivalent of acetic acid and 3 equivalents of STAB were used. After completion, the crude was purified by reverse phase chromatography using a SiliCycle SiliaSep C18 column (80 g) on Biotage isolera one (Mobile phase A: 10 mM NH₄HCO₃ in water, B: ACN) to obtain (R)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-methylpiperidin-3-yl)benzamide as a solid.

Yield: 67.2%; ¹H NMR (400 MHz, DMSO-d₆): δ 8.31-8.27 (m, 1H), 8.19 (d, J=5.6 Hz, 1H), 7.76-7.64 (m, 1H), 7.38-7.32 (m, 4H), 7.31-7.20 (m, 3H), 7.12-7.01 (m, 1H), 6.66 (d, J=5.8 Hz, 1H), 4.77-4.68 (m, 1H), 4.31-4.07 (m, 4H), 3.68 (s, 2H), 3.63-3.52 (m, 1H), 3.50-3.44 (m, 2H), 2.84-2.72 (m, 5H), 2.72-2.66 (m, 3H), 2.25-1.18 (m, 3H), 2.15-1.91 (m, 4H), 1.81-1.47 (m, 2H), 1.46-0.93 (m, 6H), three protons merged with solvent peaks; LCMS (Method A): Rt=2.04 min, m/z=706.4 [M+H]⁺, 99.72%.

### Step 5. (R)-5-Fluoro-N-isopropyl-N-(1-methylpiperidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

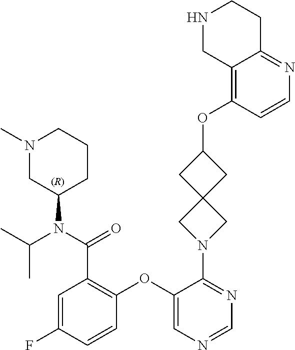

This compound was synthesized following the procedure described for the synthesis of Example 20, step 5, starting with 1 equivalent of (R)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-methylpiperidin-3-yl)benzamide, except that 0.6 equivalent of Pd-C was used. After filtration and concentration, the crude was purified by reverse phase chromatography using a SiliCycle SiliaSep C18 column (80 g) on Biotage isolera one (Mobile phase A: 10 mM NH₄HCO₃ in water, B: ACN) to obtain (R)-5-fluoro-N-isopropyl-N-(1-methylpiperidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 97%; ¹H NMR (400 MHz, DMSO-d₆): δ 8.31-8.26 (m, 1H), 8.16 (d, J=5.5 Hz, 1H), 7.76-7.64 (m, 1H), 7.31-7.21 (m, 2H), 7.13-7.01 (m, 1H), 6.64 (d, J=5.6 Hz, 1H), 4.79-4.69 (m, 1H), 4.33-4.09 (m, 4H), 3.70 (s, 2H), 3.62-3.52 (m, 1H), 3.26-3.16 (m, 1H), 2.98-2.91 (m, 2H), 2.86-2.76 (m, 4H), 2.70-2.67 (m, 2H), 2.28-1.92 (m, 6H), 1.81-1.49 (m, 4H), 1.47-1.30 (m, 3H), 1.12-0.94 (m, 3H), two protons merged with solvent peaks; LCMS (Method H): Rt=1.41 min, m/z=616.3 [M+H]⁺; HPLC (Method A): Rt=4.25 min, 97.93%; Chiral SFC (Method C): Rt=3.46 min, 100%.

### Example 50. (S)-5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(1-methylpiperidin-3-yl)benzamide (Compound No. 50)

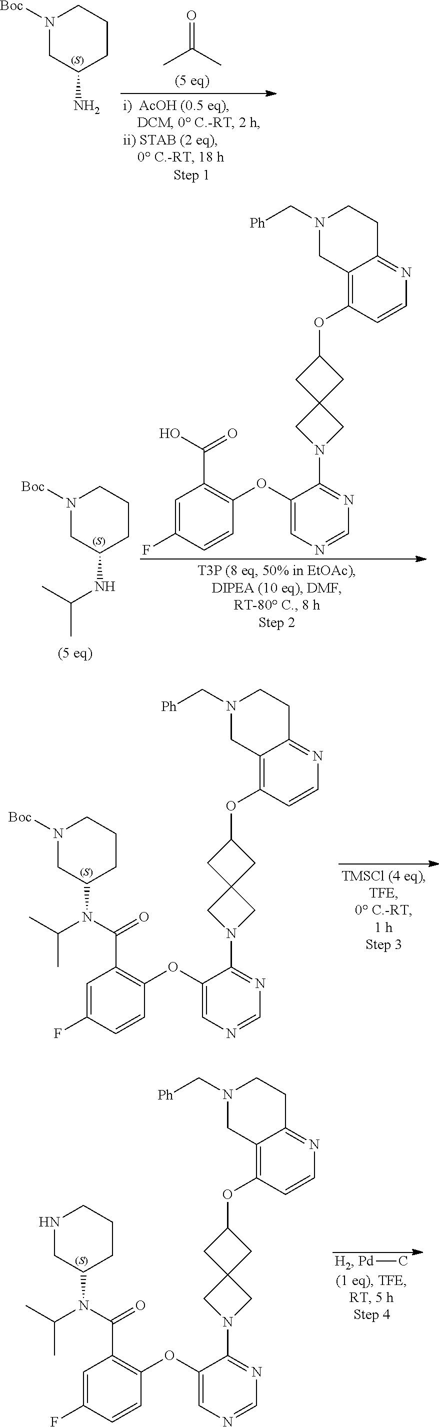

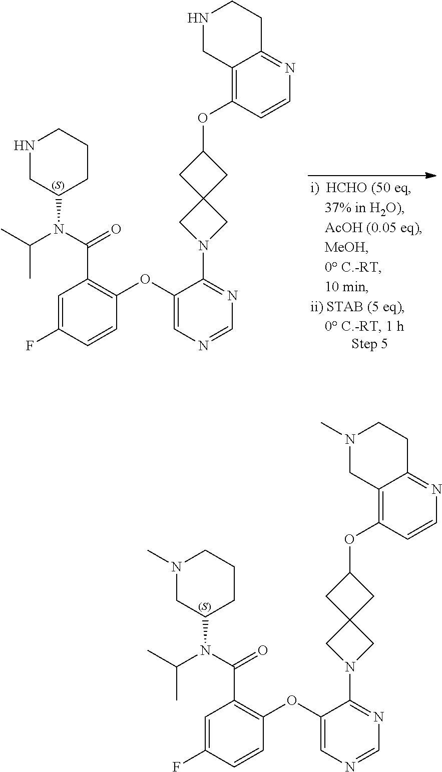

### Step 1. tert-Butyl (S)-3-(isopropylamino)piperidine-1-carboxylate

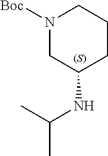

This compound was synthesized following the procedure described for the synthesis of Intermediate 1, starting with 1 equivalent of tert-butyl (S)-3-aminopiperidine-1-carboxylate, except that 5 equivalents of acetone and 0.5 equivalent of AcOH were used. After work-up, the filtrate was concentrated under reduced pressure to obtain crude tert-butyl (S)-3-(isopropylamino)piperidine-1-carboxylate as a semi-solid. This material was used without further purification.

Yield: 91%; 1H NMR (400 MHz, DMSO-d₆): δ 3.93-3.73 (m, 1H), 3.72-3.60 (m, 1H), 2.91-2.83 (m, 1H), 2.82-2.72 (m, 1H), 2.49-2.43 (m, 1H), 1.86-1.78 (m, 1H), 1.64-1.53 (m, 1H), 1.39 (s, 6H), 1.34-1.25 (m, 1H), 1.21-1.07 (m, 1H), 0.96 (dd, J=2.1, 6.3 Hz, 6H), two protons merged with solvent peaks.

### Step 2. tert-Butyl (S)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)piperidine-1-carboxylate

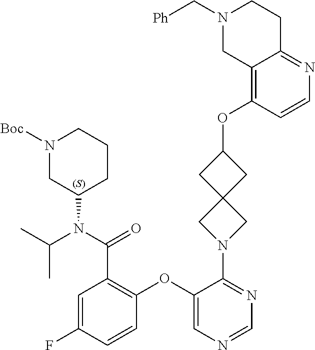

This compound was synthesized following the procedure described for the synthesis of Example 25, step 2, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 5 equivalents of tert-butyl (S)-3-(isopropylamino)piperidine-1-carboxylate, except that 8 equivalents of T3P (50% in EtOAc) and 10 equivalents of DIPEA were used. After completion, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl (S)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)piperidine-1-carboxylate as a solid.

Yield: 35.9%; LCMS (Method A): Rt=2.11 min, m/z=792.2 [M+H]⁺, 83.43%.

### Step 3. (S)-2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(piperidin-3-yl)benzamide

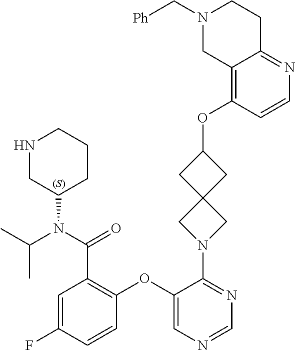

This compound was synthesized following the procedure described for the synthesis of Example 20, step 3, starting with 1 equivalent of (S)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(piperidin-3-yl)benzamide. After completion, the reaction was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain (S)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(piperidin-3-yl)benzamide as a solid.

Yield: 45.4%; ¹H NMR (400 MHz, DMSO-d₆): δ 8.31-8.26 (m, 1H), 8.19 (d, J=5.6 Hz, 1H), 7.80-7.65 (m, 1H), 7.37-7.32 (m, 4H), 7.31-7.20 (m, 3H), 7.14-6.94 (m, 1H), 6.70-6.63 (m, 1H), 4.76-4.68 (m, 1H), 4.34-4.05 (m, 4H), 3.69 (s, 2H), 3.47 (s, 2H), 2.84-2.73 (m, 5H), 2.72-2.65 (m, 4H), 2.27-2.15 (m, 4H), 2.08 (s, 2H), 1.69-1.53 (m, 2H), 1.45-1.30 (m, 4H), 1.11-0.93 (m, 4H); LCMS (Method A): Rt=1.92 min, m/z=692.3 [M+H]⁺, 99.13%.

### Step 4. (S)-5-Fluoro-N-isopropyl-N-(piperidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

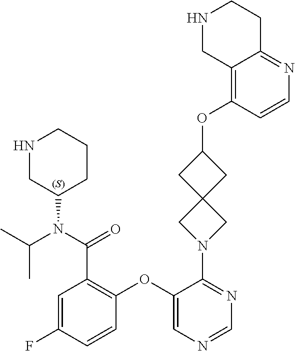

This compound was synthesized following the procedure described for the synthesis of Example 20, step 5, starting with 1 equivalent of (S)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(piperidin-3-yl)benzamide, except that 1 equivalent of Pd-C was used. After filtration and concentration, the crude was purified by reverse phase chromatography using a SiliCycle SiliaSep C18 column (80 g) on Biotage isolera one (Mobile phase A: 10 mM NH₄HCO₃ in water, B: ACN) to obtain (S)-5-fluoro-N-isopropyl-N-(piperidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 86%; LCMS (Method C): Rt=1.40 min, m/z=602.3 [M+H]⁺, 97.06%.

### Step 5. (S)—5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(1-methylpiperidin-3-yl)benzamide

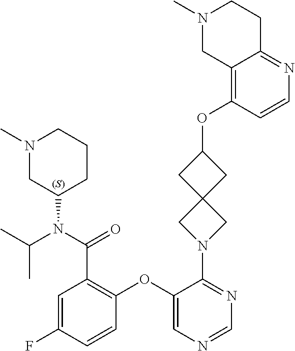

This compound was synthesized following the procedure described for the synthesis of Example 20, Step 4, starting with 1 equivalent of (S)-5-fluoro-N-isopropyl-N-(piperidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide, except that 50 equivalents of formaldehyde (37% in H₂O), 0.05 equivalent of acetic acid, and 5 equivalents of STAB were used. After completion, the crude was purified by reverse phase chromatography using a SiliCycle SiliaSep C18 column (40 g) on Biotage isolera one (Mobile phase A: 10 mM NH₄HCO₃ in water, B: ACN) to obtain (,S)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(1-methylpiperidin-3-yl)benzamide as a solid.

Yield: 54.0%; ¹H NMR (400 MHz, DMSO-d₆): δ 8.31-8.27 (m, 1H), 8.18 (d, J=5.6 Hz, 1H), 7.76-7.65 (m, 1H), 7.31-7.21 (m, 2H), 7.12-7.01 (m, 1H), 6.66 (d, J=5.6 Hz, 1H), 4.80-4.71 (m, 1H), 4.34-4.08 (mn, 4H), 3.78-3.50 (m, 1H), 3.37 (br s, 2H), 3.27-3.16 (m, 1H), 2.86-2.74 (m, 5H), 2.65-2.60 (mn, 2H), 2.37 (s, 3H), 2.30-2.21 (mn, 2H), 2.20-2.01 (mn, 3H), 2.00-1.90 (m, 1H), 1.82-1.48 (n 4H), 1.47-1.18 (N, 3H), 1.12-0.93 (m, 3H)D two protons merged with solvent peaks; LCMS (Method C): Rt=1.38 min, m/z=630.3 [M+H]⁺; HPLC (Method A): Rt=4.73 min, 98.34%; Chiral SFC (Method C): Rt=3.61 min, 100%.

### Example 51. (D1)-5-Fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)-N-((S)-1-methylpyrrolidin-3-yl)benzamide (Compound No. 51)

### Example 52. (1)₂)-5-Fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)-N-((S)-1-methylpyrrolidin-3-yl)benzamide (Compound No. 52)

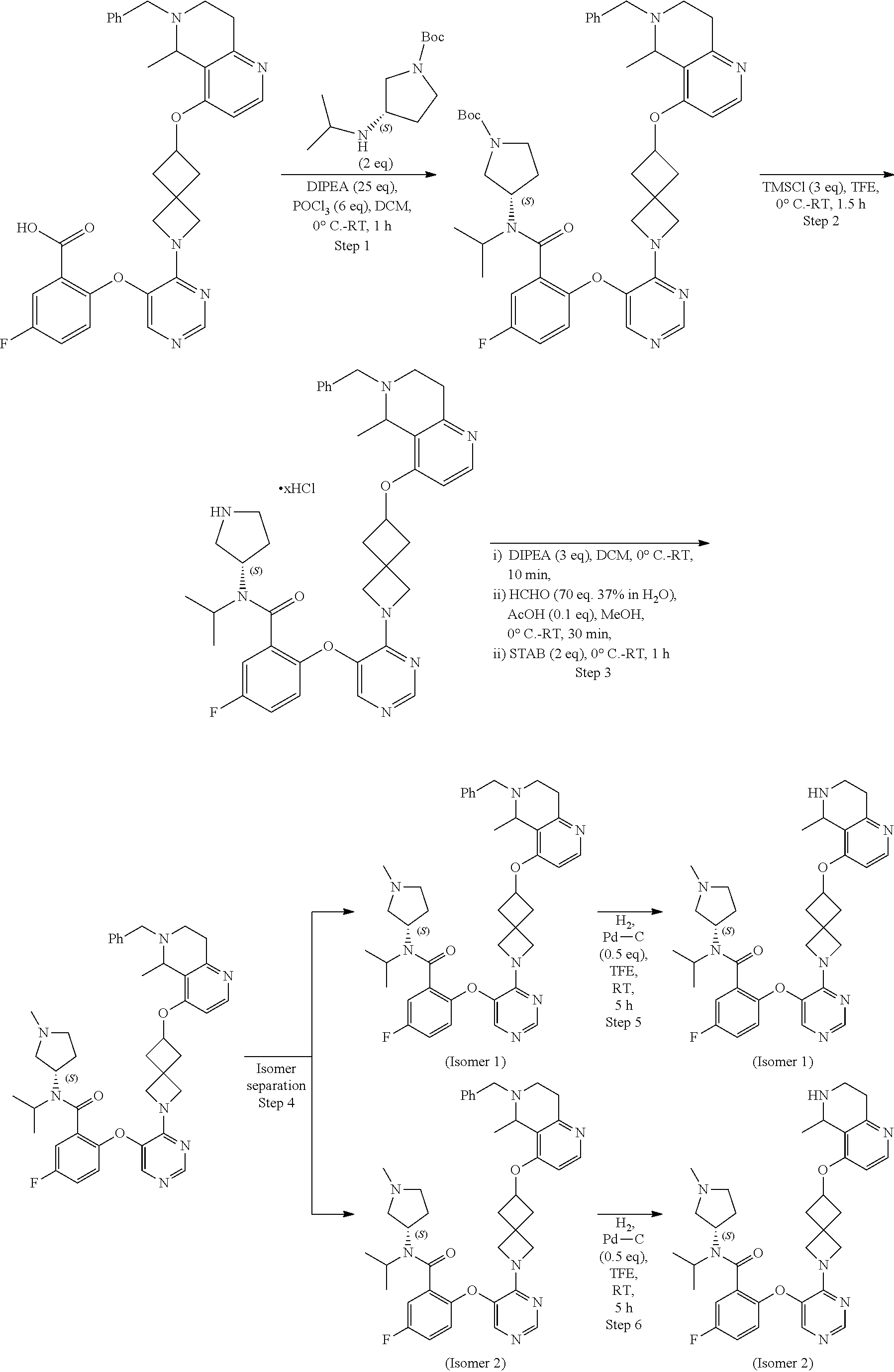

### Step 1. tert-Butyl (3S)-3-(2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)pyrrolidine-1-carboxylate

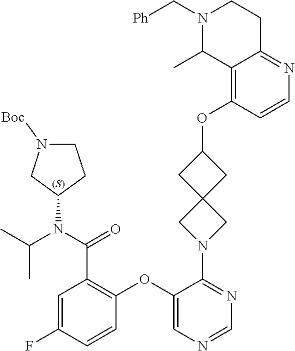

This compound was synthesized following the procedure described for the synthesis of Example 20. step 2, starting with 1 equivalent of (+)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 2 equivalents of tert-butyl (.S)-3-(isopropylamino)pyrrolidine-1-carboxylate, except that 25 equivalents of DIPEA was used. After work-up, the crude compound was purified by reverse phase chromatography using a SiliCycle SiliaSep C18 column (80 g) on Biotage isolera one (Mobile phase A: 10 mM NH₄HCO₃ in water, B: ACN) to afford tert-butyl (3S)-3-(2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)pyrrolidine-1-carboxylate as a solid.

Yield: 55.3%; LCMS (Method A): Rt=2.34 min, m/z=792.3 [M+H]⁺, 99.14%.

### Step 2. 2-((4-(6-((6-Benzyl-S-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((S)-pyrrolidin-3-yl)benzamide xHCl

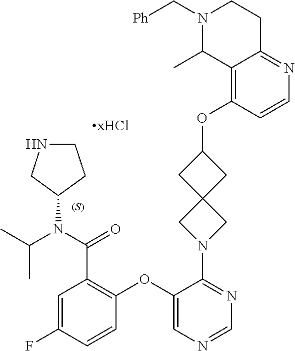

This compound was synthesized following the procedure described for the synthesis of Example 20, step 3, starting with 1 equivalent of tert-butyl (3S)-3-(2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)pyrrolidine-1-carboxylate, except that 3 equivalents of TMSCl was used. After completion, the reaction was concentrated under reduced pressure to obtain crude 2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N—((S)-pyrrolidin-3-yl)benzamide xHCl as a solid. This material was used without further purification.

Yield: 97%; LCMS (Method A): Rt=1.96 min, m/z=692.4 [M+H]⁺, 97.36%.

### Step 3. 2-((4-(6-((6-Benzyl-S-methyl-5,6,7,8-tetrahydro-1,6-naphthridine-4-yl)oxy)-2-azaspiro[3.3]heptan-2-yl)pyrimidin-5-yl)oxy)-5-fluoro-N-isopropyl-N—((S)-1-methylpyrrolidin-3-yl)benzamide

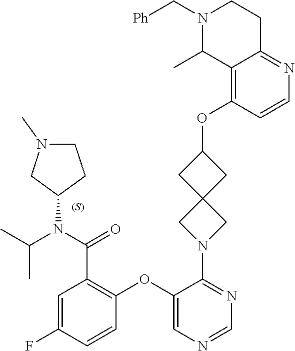

This compound was synthesized following the procedure described for the synthesis of Example 20, step 4, starting with 1 equivalent of 2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N—((S)-pyrrolidin-3-yl)benzamide xHCl, except that 3 equivalents of DIPEA, 0.1 equivalent of AcOH, 70 equivalents of formaldehyde (37% in H₂O), and 2 equivalents of STAB were used. After completion, the reaction was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain 2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N—((S)-1-methylpyrrolidin-3-yl)benzamide as a solid.

Yield: 79%; LCMS (Method A): Rt=2.07 min, m/z=706.4 [M+H]⁺, 94.97%; Chiral SFC (Method AE): Peak 1: Rt=3.63 min, 48.86%; Peak 2: Rt=4.44 min, 49.66%.

### Step 4. (D1)-2-((4-(6-((6-Benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N—((S)-1-methylpyrrolidin-3-yl)benzamide and (D2)-2-((4-(6-((6-Benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N—((S)-1-methylpyrrolidin-3-yl)benzamide

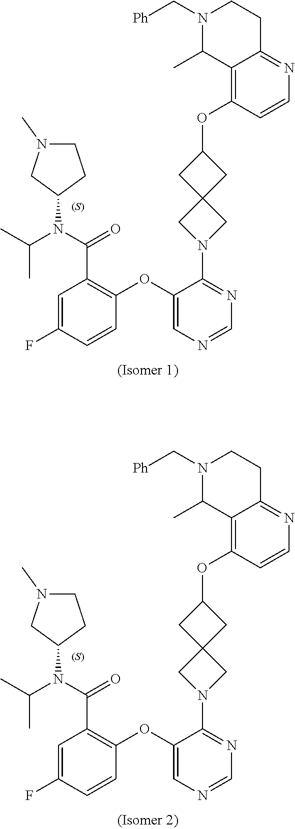

2-((4-(6-((6-Benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N—((S)-1-methylpyrrolidin-3-yl)benzamide (0.25 g, 0.336 mmol) was purified by chiral Prep SFC (Method I) to obtain (D1)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N—((S)-1-methylpyrrolidin-3-yl)benzamide (70 mg, 22.17% yield) and (D2)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N—((S)-1-methylpyrrolidin-3-yl)benzamide (70 mg, 20.45% yield) as solids.

Isomer 1. ¹H NMR (400 MHz, DMSO-d₆): δ 8.29-8.26 (m, 1H), 8.20 (d, J=5.5 Hz, 1H), 7.77-7.72 (m, 1H), 7.37-7.29 (m, 4H), 7.29-7.15 (m, 3H), 7.12-7.02 (m, 1H), 6.67 (d, J=5.4 Hz, 1H), 4.79-4.69 (m, 1H), 4.33-4.15 (m, 3H), 4.14-3.94 (m, 2H), 3.90-3.57 (m, 4H), 3.10-3.00 (m, 1H), 2.92-2.69 (m, 5H), 2.66-2.56 (m, 2H), 2.53-2.46 (m, 2H), 2.28-2.10 (m, 5H), 2.02-1.55 (m, 2H), 1.49-1.31 (m, 3H), 1.24 (d, J=6.4 Hz, 3H), 1.11-0.95 (m, 3H); LCMS (Method A): Rt=2.24 min, m/z=706.2 [M+H]⁺, 95.14%; Chiral SFC (Method AE): Rt=3.42 min, 96.10%.

Isomer 2. LCMS (Method A): Rt=2.16 min, m/z=706.2 [M+H]⁺, 87.77%; Chiral SFC (Method AE): Rt=4.36 min, 99.06%.

### Step 5. (D1)-5-Fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)-N—((S)-1-methylpyrrolidin-3-yl)benzamide

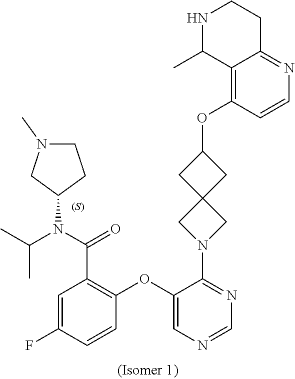

This compound was synthesized following the procedure described for the synthesis of Example 20, step 5, starting with 1 equivalent of (D1)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N—((S)-1-methylpyrrolidin-3-yl)benzamide, except that 0.5 equivalent of Pd-C was used. After filtration and concentration, the crude was purified by reverse phase chromatography using a SiliCycle SiliaSep C18 column (12 g) on Biotage isolera one (Mobile phase A: 10 mM NH₄HCO₃ in water, B: ACN) to obtain (D1)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)-N—((S)-1-methylpyrrolidin-3-yl)benzamide as a solid.

Yield: 59.5%; ¹H NMR (400 MHz, DMSO-d₆): δ 8.30-8.25 (m, 1H), 8.15 (d, J=5.6 Hz, 1H), 8.77-7.72 (m, 1H), 7.31-7.16 (m, 2H), 7.12-7.03 (m, 1H), 6.66-6.61 (m, 1H), 4.78-4.69 (m, 1H), 4.35-4.17 (m, 4H), 4.16-4.01 (m, 2H), 3.90-3.65 (m, 2H), 3.12-3.02 (m, 1H), 2.93-2.70 (m, 6H), 2.69-2.62 (m, 2H), 2.30-2.11 (m, 6H), 2.06-1.73 (m, 2H), 1.50-1.32 (m, 3H), 1.28 (d, J=6.5 Hz, 3H), 1.12-0.96 (m, 3H); LCMS (Method C): Rt=1.28 min, m/z=616.3 [M+H]⁺; HPLC (Method A): Rt=4.07 min, 99.22%; Chiral SFC (Method Y): Rt=5.03 min, 97.60%.

### Step 6. (D2)-5-Fluoro-N-isopropyl-2-((4-(6-((S-methyl-5,6,7,8-tetrahydro-1,6-naphthyridin-4-yl)oxy)-2-azaspiro[3.3]heptan-2-yl)pyrimidin-5-yl)oxy)-N—((S)-1-methylpyrrolidin-3-yl)benzamide

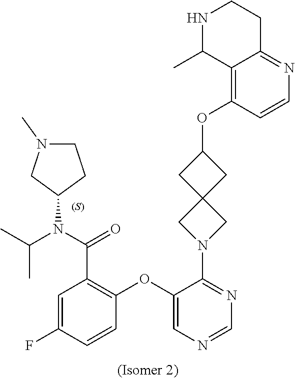

This compound was synthesized following the procedure described for the synthesis of Example 20, step 5, starting with 1 equivalent of (D2)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N—((S)-1-methylpyrrolidin-3-yl)benzamide, except that 0.5 equivalent of Pd-C was used. After filtration and concentration, the crude was purified by reverse phase chromatography using a SiliCycle SiliaSep C18 column (12 g) on Biotage isolera one (Mobile phase A: 10 mM NH₄HCO₃ in water, B: ACN) to obtain (D2)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)-N—((S)-1-methylpyrrolidin-3-yl)benzamide as a solid.

Yield: 53.8%; ¹H NMR (400 MHz, DMSO-d₆): δ 8.30-8.25 (m, 1H), 8.15 (d, J=5.6 Hz, 1H), 7.78-7.72 (m, 1H), 7.31-7.16 (m, 2H), 7.12-7.03 (m, 1H), 6.66-6.61 (m, 1H), 4.74 (quin, J=6.4 Hz, 1H), 4.34-4.18 (m, 4H), 4.15-4.02 (m, 2H), 3.90-3.65 (m, 2H), 3.12-3.01 (m, 1H), 2.92-2.85 (m, 2H), 2.85-2.69 (m, 4H), 2.66-2.54 (m, 2H), 2.29-2.10 (m, 6H), 2.07-1.57 (m, 2H), 1.49-1.32 (m, 3H), 1.30-1.23 (m, 3H), 1.12-0.96 (m, 3H); LCMS (Method C): Rt=1.41 min, m/z=616.3 [M+H]⁺; HPLC (Method A): Rt=4.05 min, 99.61%; Chiral SFC (Method Y): Rt=6.10 min, 98.12%.

### Example 53. (D1)-N-((1r,3r)-3-(Dimethylamino)-3-methylcyclobutyl)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide (Compound No. 53)

### Example 54. (D2)-N-((1r,3r)-3-(Dimethylamino)-3-methylcyclobutyl)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide (Compound No. 54)

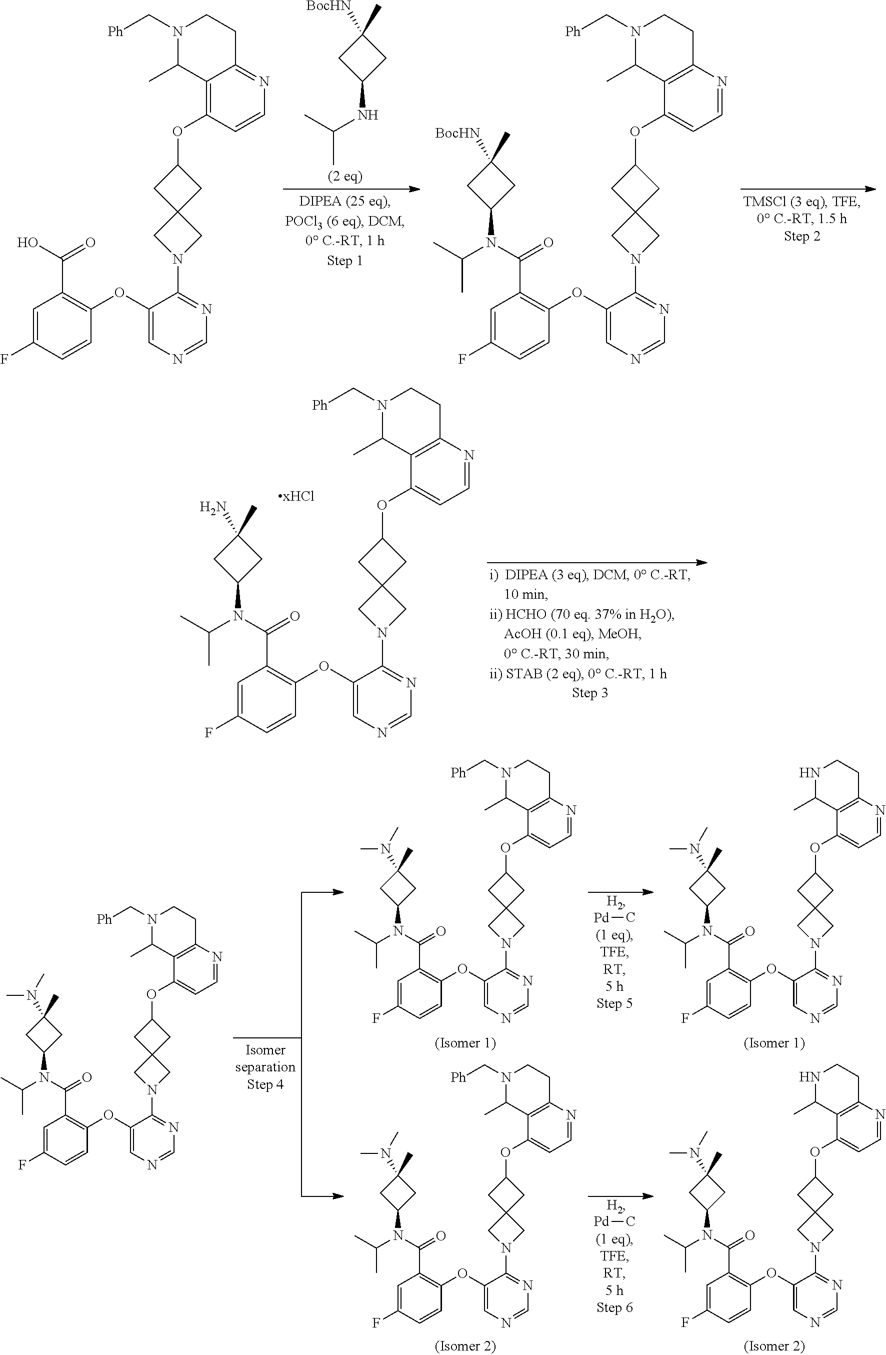

### Step 1. tert-Butyl ((1r,3r)-3-(2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-1-methylcyclobutyl)carbamate

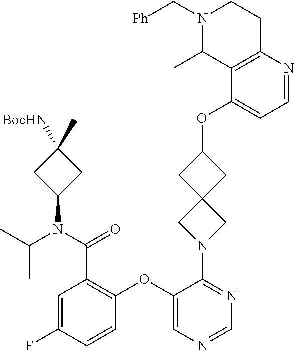 [SMILES: CC1c2c(OC3CC4(C3)CN(c3ncncc3Oc3ccc(F)cc3C(=O)N(C(C)C)[C@H]3C[C@](C)(NC(=O)OC(C)(C)C)C3)C4)ccnc2CCN1Cc1ccccc1]

This compound was synthesized following the procedure described for the synthesis of Example 20, step 2, starting with 1 equivalent of (±)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 2 equivalents of tert-butyl ((1r,3r)-3-(isopropylamino)-1-methylcyclobutyl)carbamate, except that 25 equivalents of DIPEA was used. After work-up, the crude compound was purified by reverse phase chromatography using a SiliCycle SiliaSep C18 column (80 g) on Biotage isolera one (Mobile phase A: 10 mM NH₄HCO₃ in water, B: ACN) to afford tert-butyl ((1r,3r)-3-(2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-1-methylcyclobutyl)carbamate as a solid.

Yield: 53.8%; LCMS (Method A): Rt=2.38 min, m/z=806.3 [M+H]⁺, 93.20%.

### Step 2. N-((1r,3r)-3-Amino-3-methylcyclobutyl)-2-((4-(6-((6-ben yl-S-methyl-5,6,7,8-tetrahydro-1,6-naphthyridin-4-yl)oxy)-2-azaspiro[3.3]heptan-2-yl)pyrimidin-5yl)oxy)-5-fluoro-N-isopropylbenzamide xHCl

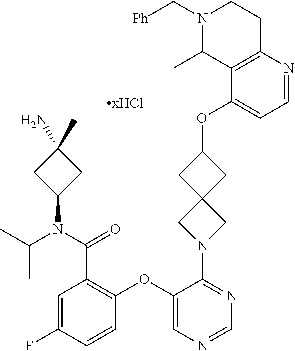 [SMILES: CC1c2c(OC3CC4(C3)CN(c3ncncc3Oc3ccc(F)cc3C(=O)N(C(C)C)[C@H]3C[C@](C)(N)C3)C4)ccnc2CCN1Cc1ccccc1]

This compound was synthesized following the procedure described for the synthesis of Example 20, step 3, starting with 1 equivalent of tert-butyl ((1r,3r)-3-(2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-1-methylcyclobutyl)carbamate. After completion, the reaction was concentrated under reduced pressure to obtain crude N-((1r,3r)-3-amino-3-methylcyclobutyl)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl as a solid. This material was used without further purification.

Yield: 95%; LCMS (Method A): Rt=1.95 min, m/z=706.4 [M+H]⁺, 96.97%.

### Step 3. 2-((4-(6-((6-Benzyl-S-methyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide

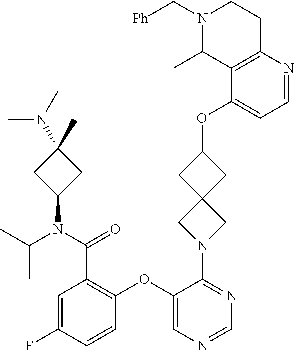 [SMILES: CC1c2c(OC3CC4(C3)CN(c3ncncc3Oc3ccc(F)cc3C(=O)N(C(C)C)[C@H]3C[C@](C)(N(C)C)C3)C4)ccnc2CCN1Cc1ccccc1]

This compound was synthesized following the procedure described for the synthesis of Example 20, step 4, starting with 1 equivalent of N-((1r,3r)-3-amino-3-methylcyclobutyl)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl, except that 3 equivalents of DIPEA, 0.1 equivalent of AcOH, 70 equivalents of formaldehyde (37% in H₂O), and 2 equivalents of STAB were used. After completion, the reaction was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain 2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide as a solid.

Yield: 90%; ¹H NMR (400 MHz, DMSO-d₆): δ 8.27 (d, J=6.4 Hz, 1H), 8.19 (d, J=5.3 Hz, 1H), 7.76-7.59 (m, 1H), 7.36-7.29 (m, 4H), 7.29-7.21 (m, 3H), 7.17-7.05 (m, 1H), 6.70-6.64 (m, 1H), 4.74 (quin, J=6.6 Hz, 1H), 4.34-4.08 (m, 4H), 4.04-3.93 (m, 1H), 3.84-3.56 (m, 4H), 3.11-3.00 (m, 1H), 2.93-2.66 (m, 5H), 2.65-2.60 (m, 1H), 2.26-2.13 (m, 2H), 2.12-1.87 (m, 8H), 1.80-1.68 (m, 1H), 1.46-1.28 (m, 3H), 1.24 (d, J=6.5 Hz, 3H), 1.13-0.90 (m, 6H); LCMS (Method A): Rt=2.22 min, m/z=734.4 [M+H]⁺, 98.05%; Chiral SFC (Method AG): Peak 1: Rt=3.94 min, 48.65%; Peak 2: Rt=4.72 min, 50.23%.

### Step 4. (D1)-2-((4-(6-((6-Benzyl-5-methyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide and (D2)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide

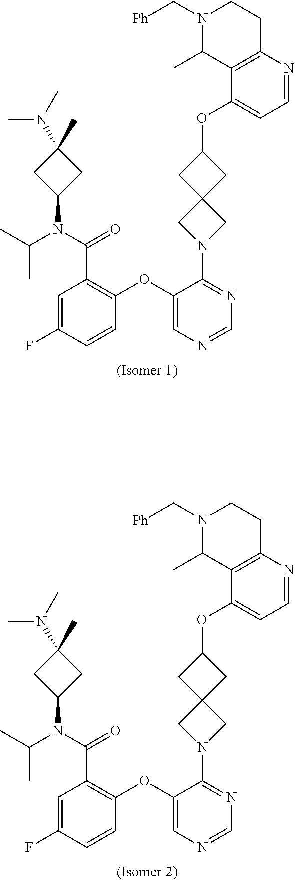 [SMILES: CC1c2c(OC3CC4(C3)CN(c3ncncc3Oc3ccc(F)cc3C(=O)N(C(C)C)[C@H]3C[C@](C)(N(C)C)C3)C4)ccnc2CCN1Cc1ccccc1 ; CC1c2c(OC3CC4(C3)CN(c3ncncc3Oc3ccc(F)cc3C(=O)N(C(C)C)[C@H]3C[C@](C)(N(C)C)C3)C4)ccnc2CCN1Cc1ccccc1]

2-((4-(6-((6-Benzyl-5-methyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide (0.3 g, 0.401 mmol) was purified by chiral Prep SFC (Method J) to obtain (D1)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide (50 mg, 14.63% yield) and (D2)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide (70 mg, 20.80% yield) as solids.

Isomer 1. ¹H NMR (400 MHz, DMSO-d₆): δ 8.27 (d, J=6.3 Hz, 1H), 8.20 (d, J=5.3 Hz, 1H), 7.77-7.58 (m, 1H), 7.37-7.29 (m, 4H), 7.29-7.21 (m, 3H), 7.17-7.03 (m, 1H), 6.70-6.63 (m, 1H), 4.78-4.69 (m, 1H), 4.35-4.08 (m, 4H), 4.03-3.93 (m, 1H), 3.85-3.55 (m, 4H), 3.11-2.98 (m, 1H), 2.92-2.69 (m, 5H), 2.65-2.56 (m, 1H), 2.26-2.14 (m, 2H), 2.12-1.88 (m, 8H), 1.81-1.66 (m, 1H), 1.46-1.27 (m, 3H), 1.24 (d, J=6.4 Hz, 3H), 1.14-0.90 (m, 6H); LCMS (Method A): Rt=2.24 min, m/z=734.4 [M+H]⁺, 95.42%; Chiral SFC (Method AE): Rt=2.82 min, 98.63%.

Isomer 2. ¹H NMR (400 MHz, DMSO-d₆): δ 8.27 (d, J=6.4 Hz, 1H), 8.19 (d, J=5.4 Hz, 1H), 7.77-7.58 (m, 1H), 7.36-7.29 (m, 4H), 7.29-7.21 (m, 3H), 7.17-7.05 (m, 1H), 6.71-6.62 (m, 1H), 4.74 (quin, J=6.8 Hz, 1H), 4.34-4.08 (m, 4H), 4.02-3.93 (m, 1H), 3.84-3.56 (m, 4H), 3.10-3.00 (m, 1H), 2.93-2.69 (m, 5H), 2.64-2.56 (m, 1H), 2.26-2.14 (m, 2H), 2.13-1.89 (m, 8H), 1.80-1.68 (m, 1H), 1.46-1.27 (m, 3H), 1.24 (d, J=6.5 Hz, 3H), 1.14-0.90 (m, 6H); LCMS (Method A): Rt=2.23 min, m/z=734.2 [M+H]⁺, 96.93%; Chiral SFC (Method AE): Rt=3.50 min, 98.98%.

### Step 5. (D1)-N-((1r,3r)-3-(Dimethylamino)-3-methylcyclobutyl)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide

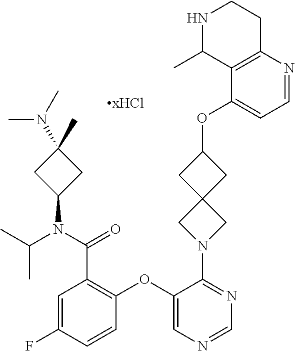 [SMILES: CC1NCCc2nccc(OC3CC4(C3)CN(c3ncncc3Oc3ccc(F)cc3C(=O)N(C(C)C)[C@H]3C[C@](C)(N(C)C)C3)C4)c21]

This compound was synthesized following the procedure described for the synthesis of Example 20, step 5, starting with 1 equivalent of (D1)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide, except that 0.5 equivalent of Pd-C was used. After filtration and concentration, the crude was purified by reverse phase chromatography using a SiliCycle SiliaSep C18 column (12 g) on Biotage isolera one (Mobile phase A: 10 mM NH₄HCO₃ in water, B: ACN) to obtain (D1)-N-((1r,3r)-3-(dimethylamino)-3-methylcyclobutyl)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 93%; ¹H NMR (400 MHz, DMSO-d₆): δ 8.27 (d, J=6.8 Hz, 1H), 8.15 (d, J=5.5 Hz, 1H), 7.79-7.57 (m, 1H), 7.32-7.21 (m, 2H), 7.18-7.05 (m, 1H), 6.67-6.59 (m, 1H), 4.74 (quin, J=6.4 Hz, 1H), 4.36-4.11 (m, 4H), 4.09-3.93 (m, 2H), 3.85-3.63 (m, 2H), 3.12-3.02 (m, 1H), 2.93-2.73 (m, 3H), 2.70-2.64 (m, 2H), 2.28-2.19 (m, 2H), 2.16-1.89 (m, 9H), 1.80-1.68 (m, 1H), 1.47-1.25 (m, 6H), 1.16-0.90 (m, 6H); LCMS (Method C): Rt=1.38 min, m/z=644.5 [M+H]⁺; HPLC (Method A): Rt=4.49 min, 99.41%; Chiral SFC (Method AH): Rt=3.07 min, 98.18%.

### Step 6. (D2)-N-((1r,3r)-3-(Dimethylamino)-3-methylcyclobutyl)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide

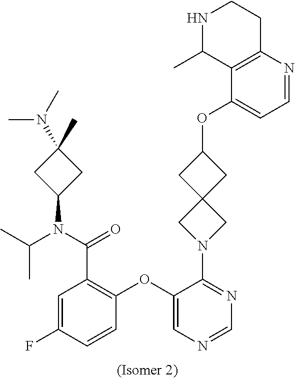 [SMILES: CC1NCCc2nccc(OC3CC4(C3)CN(c3ncncc3Oc3ccc(F)cc3C(=O)N(C(C)C)[C@H]3C[C@](C)(N(C)C)C3)C4)c21]

This compound was synthesized following the procedure described for the synthesis of Example 20, step 5, starting with 1 equivalent of (D2)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide, except that 0.5 equivalent of Pd-C was used. After filtration and concentration, the crude was purified by reverse phase chromatography using a SiliCycle SiliaSep C18 column (12 g) on Biotage isolera one (Mobile phase A: 10 mM NH₄HCO₃ in water, B: ACN) to obtain (D2)-N-((1r,3r)-3-(dimethylamino)-3-methylcyclobutyl)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 40.5%; ¹H NMR (400 MHz, DMSO-d₆): δ 8.27 (d, J=6.8 Hz, 1H), 8.15 (d, J=5.3 Hz, 1H), 7.77-7.59 (m, 1H), 7.32-7.21 (m, 2H), 7.18-7.05 (m, 1H), 6.67-6.58 (m, 1H), 4.74 (quin, J=6.5 Hz, 1H), 4.36-4.11 (m, 4H), 4.09-3.93 (m, 2H), 3.86-3.63 (m, 2H), 3.12-3.02 (m, 1H), 2.93-2.85 (m, 1H), 2.85-2.74 (m, 2H), 2.73-2.65 (m, 2H), 2.28-2.18 (m, 2H), 2.17-1.88 (m, 9H), 1.80-1.68 (m, 1H), 1.47-1.24 (m, 6H), 1.15-0.91 (m, 6H); LCMS (Method C): Rt=1.37 min, m/z=644.3 [M+H]⁺; HPLC (Method A): Rt=4.48 min, 99.49%; Chiral SFC (Method AH): Rt=3.93 min, 97.96%.

### Example 55. (D1)-5-Fluoro-N-isopropyl-N-(3-(1-methylazetidin-3-yl)cyclobutyl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 55)

### Example 56. (D2)-5-Fluoro-N-isopropyl-N-(3-(1-methylazetidin-3-yl)cyclobutyl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 56)

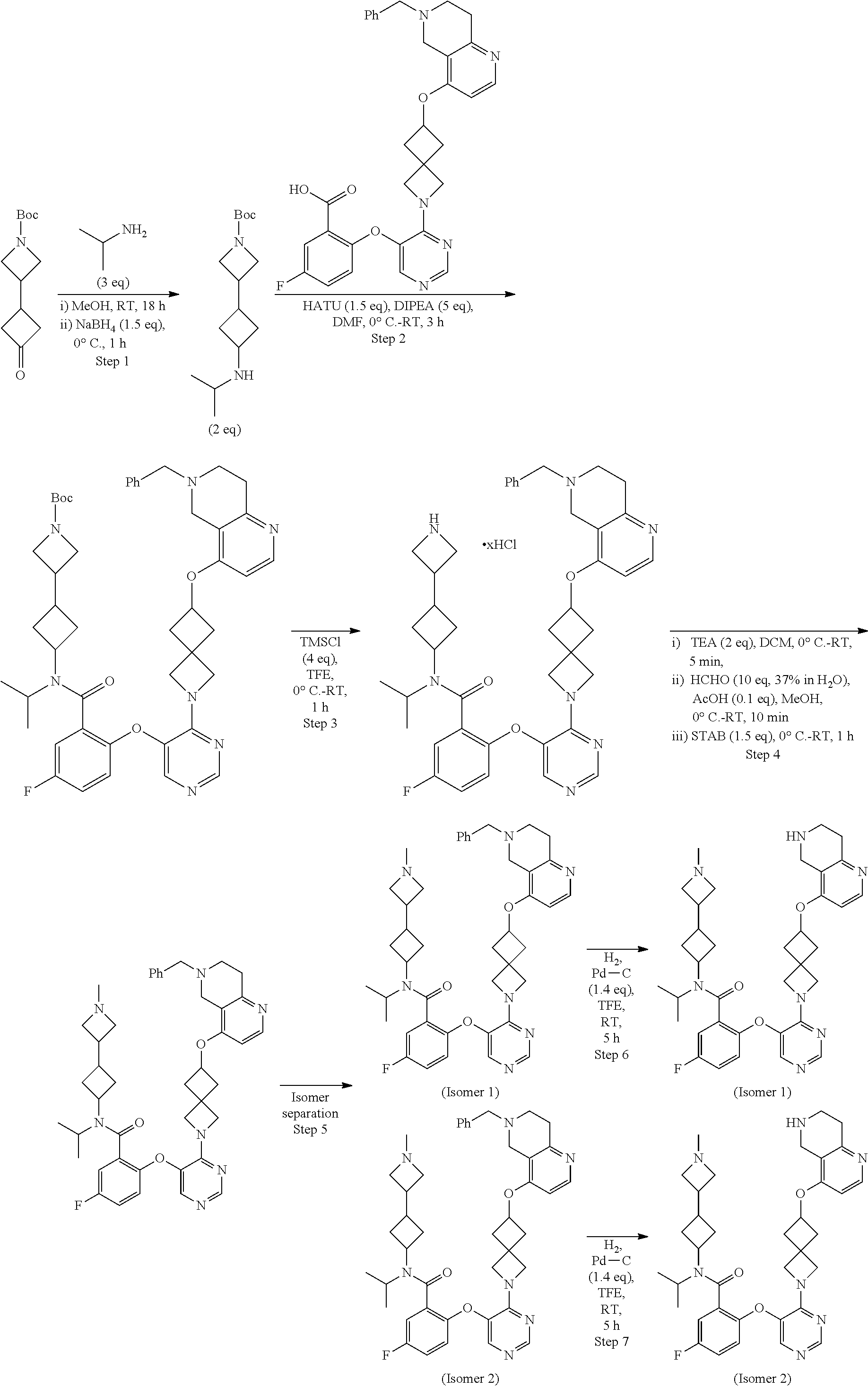

### Step 1. tert-Butyl 3-(3-(isopropylamino)cyclobutyl)azetidine-1-carboxylate Boc

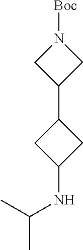 [SMILES: CC(C)NC1CC(C2CN(C(=O)OC(C)(C)C)C2)C1]

This compound was synthesized following the procedure described for the synthesis of Example 36, Step 1, starting with 1 equivalent of tert-butyl 3-(3-oxocyclobutyl)azetidine-1-carboxylate, except that 3 equivalents of isopropylamine was used. After completion, the reaction was concentrated under reduced pressure. The residue was diluted with water and the mixture was extracted with DCM. The combined organic layer was dried over sodium sulfate and filtered, and the filtrate was concentrated under reduced pressure to obtain crude tert-butyl 3-(3-(isopropylamino)cyclobutyl)azetidine-1-carboxylate as a liquid. This material was without further purification.

Yield: 84%; ¹H NMR (400 MHz, DMSO-d₆): δ 4.00-3.72 (m, 2H), 3.59-3.21 (m, 4H), 3.10-2.97 (m, 1H), 2.75-2.58 (m, 1H), 2.23-2.14 (m, 1H), 2.12-1.99 (m, 1H), 1.84-1.74 (m, 1H), 1.37 (s, 9H), 1.34-1.20 (m, 2H), 0.97-0.87 (m, 6H).

### Step 2. tert-Butyl 3-(3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclobutyl)azetidine-1-carboxylate

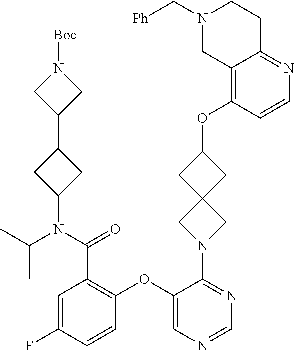 [SMILES: CC(C)N(C(=O)c1cc(F)ccc1Oc1cncnc1N1CC2(CC(Oc3ccnc4c3CN(Cc3ccccc3)CC4)C2)C1)C1CC(C2CN(C(=O)OC(C)(C)C)C2)C1]

This compound was synthesized following the procedure described for the synthesis of Example 36, Step 2, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid, except that 2 equivalents of tert-butyl 3-(3-(isopropylamino)cyclobutyl)azetidine-1-carboxylate and 5 equivalents of DIPEA were used. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl 3-(3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclobutyl)azetidine-1-carboxylate as a solid.

Yield: 41.0%; ¹H NMR (400 MHz, DMSO-d₆): δ 8.29-8.24 (m, 1H), 8.19 (d, J=5.6 Hz, 1H), 7.81-7.68 (m, 1H), 7.38-7.32 (m, 4H), 7.31-7.02 (m, 4H), 6.66 (d, J=5.6 Hz, 1H), 4.79-4.64 (m, 1H), 4.38-4.04 (m, 4H), 3.96-3.75 (m, 3H), 3.68 (s, 3H), 3.47 (s, 2H), 2.85-2.64 (m, 8H), 2.27-2.16 (m, 3H), 2.04-1.85 (m, 1H), 1.81-1.63 (m, 1H), 1.48-1.23 (m, 13H), 1.12-0.91 (m, 4H), one proton merged with solvent peaks; LCMS (Method A): Rt=2.41 min, m/z=818.3 [M+H]⁺, 98.36%.

### Step 3. N-(3-(Azetidin-3-yl)cyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl

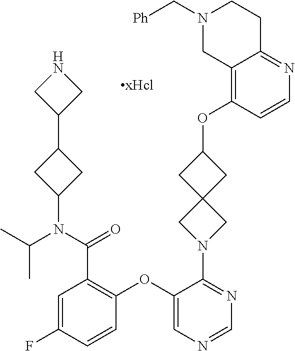 [SMILES: CC(C)N(C(=O)c1cc(F)ccc1Oc1cncnc1N1CC2(CC(Oc3ccnc4c3CN(Cc3ccccc3)CC4)C2)C1)C1CC(C2CNC2)C1]

This compound was synthesized following the procedure described for the synthesis of Example 20, Step 3, starting with 1 equivalent of tert-butyl 3-(3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)cyclobutyl)azetidine-1-carboxylate. After completion, the reaction was concentrated under reduced pressure and the residue was triturated with EtOAc. The supernatant was decanted and the resulting residue was dried under reduced pressure to obtain crude N-(3-(azetidin-3-yl)cyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl as a solid. This material was used without further purification.

Yield: 93%; LCMS (Method A): Rt=1.91 min, m/z=718.4 [M+H]⁺, 93.35%.

### Step 4. 2-((4-(6-((6-Benzyl-5,6,7,8-tetrahydro-1,6-naphthyridin-4-yl]oxy)-2-azaspiro[3.3]heptan-2-yl)pyrimidin-5-yl)oxy)-5-fluoro-N-isopropyl-N-(3-(1-methylazetidin-3-yl)cyclobutyl)benzamide

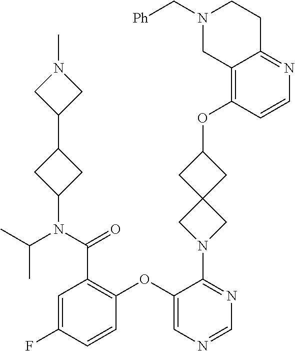 [SMILES: CC(C)N(C(=O)c1cc(F)ccc1Oc1cncnc1N1CC2(CC(Oc3ccnc4c3CN(Cc3ccccc3)CC4)C2)C1)C1CC(C2CN(C)C2)C1]

This compound was synthesized following the procedure described for the synthesis of Example 20, Step 4, starting with 1 equivalent of N-(3-(azetidin-3-yl)cyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide hydrochloride, except that 1 equivalent of AcOH and 1.5 equivalents of STAB were used. Crude 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(3-(1-methylazetidin-3-yl)cyclobutyl)benzamide was obtained as a solid. This material was used without further purification.

Yield: 81%; LCMS (Method A): Rt=2.08 min, m/z=732.4 [M+H]⁺, 95.96%.

### Step 5. (D1)-2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(3-(1-methylazetidin-3-yl)cyclobutyl)benzamide and (D2)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(3-(1-methylazetidin-3-yl)cyclobutyl)benzamide

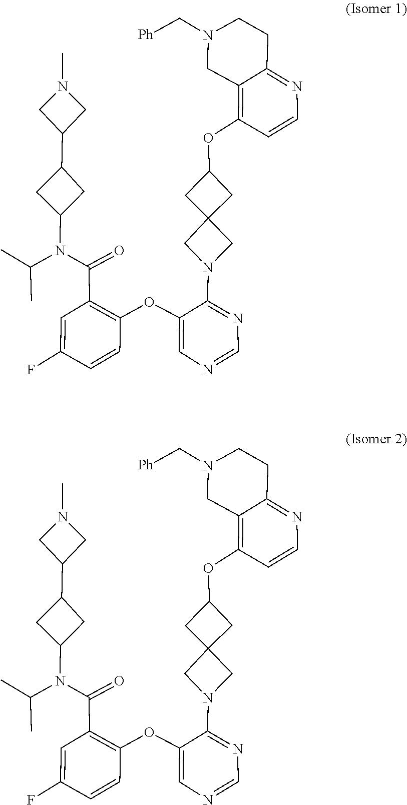 [SMILES: CC(C)N(C(=O)c1cc(F)ccc1Oc1cncnc1N1CC2(CC(Oc3ccnc4c3CN(Cc3ccccc3)CC4)C2)C1)C1CC(C2CN(C)C2)C1 ; CC(C)N(C(=O)c1cc(F)ccc1Oc1cncnc1N1CC2(CC(Oc3ccnc4c3CN(Cc3ccccc3)CC4)C2)C1)C1CC(C2CN(C)C2)C1]

2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(3-(1-methylazetidin-3-yl)cyclobutyl)benzamide (300 mg, 0.41 mmol) was purified by chiral SFC (Method G) to obtain 5 (D1)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(3-(1-methylazetidin-3-yl)cyclobutyl)benzamide (130 mg, 43% yield) and (D2)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(3-(1-methylazetidin-3-yl)cyclobutyl)benzamide (60 mg, 19.92% yield) as solids.

Isomer 1. ¹H NMR (400 MHz, DMSO-d₆): δ 8.30-8.24 (m, 1H), 8.19 (d, J=5.6 Hz, 1H), 7.79-7.70 (m, 1H), 7.39-7.32 (m, 4H), 7.31-7.04 (m, 4H), 6.68-6.63 (m, 1H), 4.79-4.65 (m, 1H), 4.32-4.07 (m, 4H), 3.81-3.74 (m, 1H), 3.68 (s, 2H), 3.47 (s, 2H), 3.21-3.12 (m, 2H), 2.85-2.74 (m, 5H), 2.71-2.66 (m, 5H), 2.28-2.17 (m, 2H), 2.13-2.10 (m, 2H), 2.08 (s, 3H), 2.05-1.98 (m, 1H), 1.93-1.84 (m, 1H), 1.77-1.65 (m, 1H), 1.47-0.93 (m, 6H); LCMS (Method A): Rt=2.06 min, m/z=732.20 [M+H]⁺; HPLC (Method A): Rt=6.10 min, 99.17%; Chiral SFC (Method AB): Rt=3.57 min, 99.20%.

Isomer 2. LCMS (Method A): Rt=2.00 min, m/z=732.4 [M+H]⁺, 99.60%; Chiral SFC (Method AB): Rt=2.95 min, 100%.

### Step 6. (D1)-5-Fluoro-N-isopropyl-N-(3-(1-methylazetidin-3-yl)cyclobutyl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

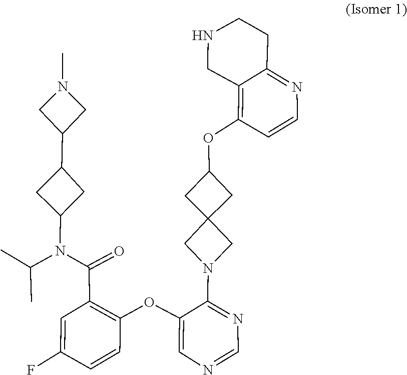 [SMILES: CC(C)N(C(=O)c1cc(F)ccc1Oc1cncnc1N1CC2(CC(Oc3ccnc4c3CNCC4)C2)C1)C1CC(C2CN(C)C2)C1]

This compound was synthesized following the procedure described for the synthesis of Example 20, Step 5, starting with 1 equivalent of (D1)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(3-(1-methylazetidin-3-yl)cyclobutyl)benzamide, except that 1.4 equivalents of Pd-C was used. After filtration and concentration, the crude was purified by reverse phase chromatography using a SiliCycle SiliaSep C18 column (40 g) on Biotage isolera one (Mobile phase A: 10 mM NH₄HCO₃ in water, B: ACN) to obtain (D1)-5-fluoro-N-isopropyl-N-(3-(1-methylazetidin-3-yl)cyclobutyl)-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 54.2%; ¹H NMR (400 MHz, DMSO-d₆): δ 8.30-8.24 (m, 1H), 8.16 (d, J=5.6 Hz, 1H), 7.82-7.71 (m, 1H), 7.33-7.01 (m, 3H), 6.69-6.60 (m, 1H), 4.72 (quin, J=6.5 Hz, 1H), 4.30-4.10 (m, 4H), 3.87-3.67 (m, 4H), 3.12 (br t, J=6.6 Hz, 2H), 2.96-2.92 (m, 2H), 2.85-2.73 (m, 4H), 2.69-2.62 (m, 4H), 2.45-2.39 (m, 2H), 2.27-2.19 (m, 2H), 2.10 (s, 3H), 2.05-1.98 (m, 1H), 1.93-1.84 (m, 1H), 1.74-1.61 (m, 1H), 1.43-0.94 (m, 6H); LCMS (Method C): Rt=1.34 min, m/z=642.3 [M+H]⁺; HPLC (Method A): Rt=4.15 min, 99.61%; Chiral SFC (Method L): Rt=2.33 min, 100%.

### Step 7. (D2)-5-Fluoro-N-isopropyl-N-(3-(1-methylazetidin-3-yl)cyclobutyl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

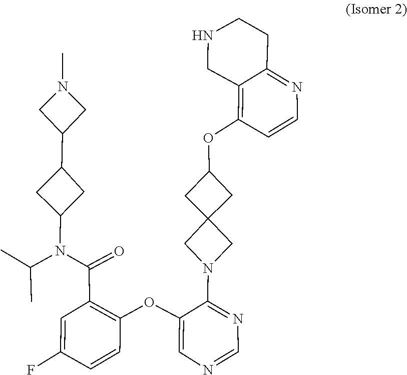 [SMILES: CC(C)N(C(=O)c1cc(F)ccc1Oc1cncnc1N1CC2(CC(Oc3ccnc4c3CNCC4)C2)C1)C1CC(C2CN(C)C2)C1]

This compound was synthesized following the procedure described for the synthesis of Example 20, Step 5, starting with 1 equivalent of (D2)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(3-(1-methylazetidin-3-yl)cyclobutyl)benzamide, except that 1.4 equivalents of Pd-C was used. After work-up, the crude was purified by reverse phase chromatography using a SiliCycle SiliaSep C18 column (12 g) on Biotage isolera one (Mobile phase A: 10 mM NH₄HCO₃ in water, B: ACN) to obtain (D2)-5-fluoro-N-isopropyl-N-(3-(1-methylazetidin-3-yl)cyclobutyl)-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 37.8%; ¹H NMR (400 MHz, DMSO-d₆): δ 8.32-8.23 (m, 1H), 8.16 (d, J=5.6 Hz, 1H), 7.78-7.64 (m, 1H), 7.34-6.99 (m, 3H), 6.64 (br d, J=5.5 Hz, 1H), 4.73 (quin, J=6.2 Hz, 1H), 4.39-3.90 (m, 5H), 3.84-3.61 (m, 3H), 3.26-3.21 (m, 2H), 3.15-3.03 (m, 2H), 2.95 (br t, J=5.6 Hz, 2H), 2.83-2.73 (m, 2H), 2.71-2.64 (m, 3H), 2.29-2.20 (m, 3H), 2.18-2.07 (m, 3H), 1.85-1.66 (m, 2H), 1.61-1.49 (m, 1H), 1.49-0.91 (m, 6H), two protons merged with solvent peaks; LCMS (Method H): Rt=1.37 min, m/z=642.3 [M+H]⁺, 98.64%; HPLC (Method A): Rt=4.08 min, 99.37%; Chiral SFC (Method N): Rt=1.57 min, 100%.

### Example 57. (D1)-N-((1r,3r)-3-(Dimethylamino)-3-methylcyclobutyl)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamide (Compound No. 57)

### Example 58. (D2)-N-((1r,3r)-3-(Dimethylamino)-3-methylcyclobutyl)-5-fluoro-2-((4-(6-((5,6,8,9,10,10a-hexahydropyrrolo[2,1-f[1,6]naphthyridin-1-yl)oxy)-2-azaspiro[3.3]heptan-2-yl)pyrimidin-5-yl)oxy)-N-isopropylbenzamide (Compound No. 58)

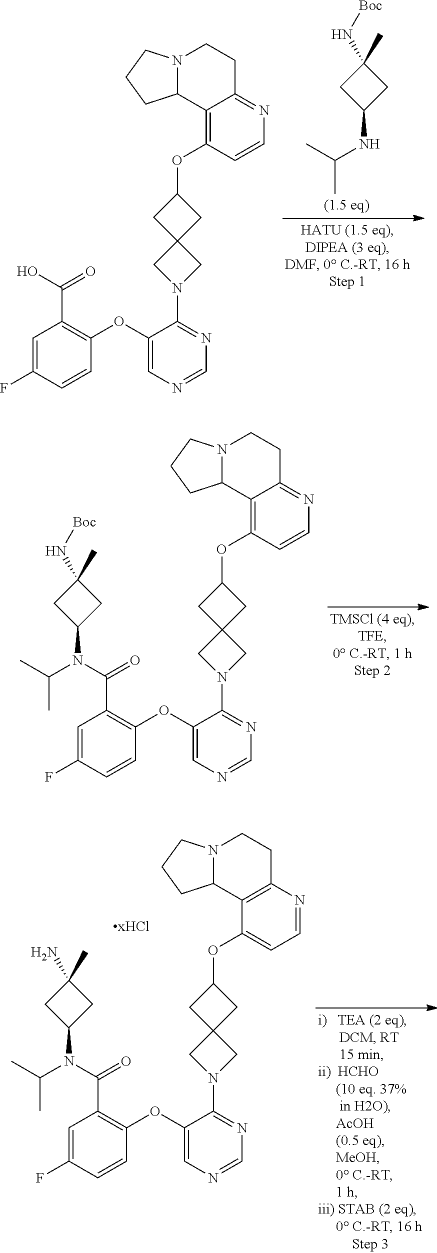

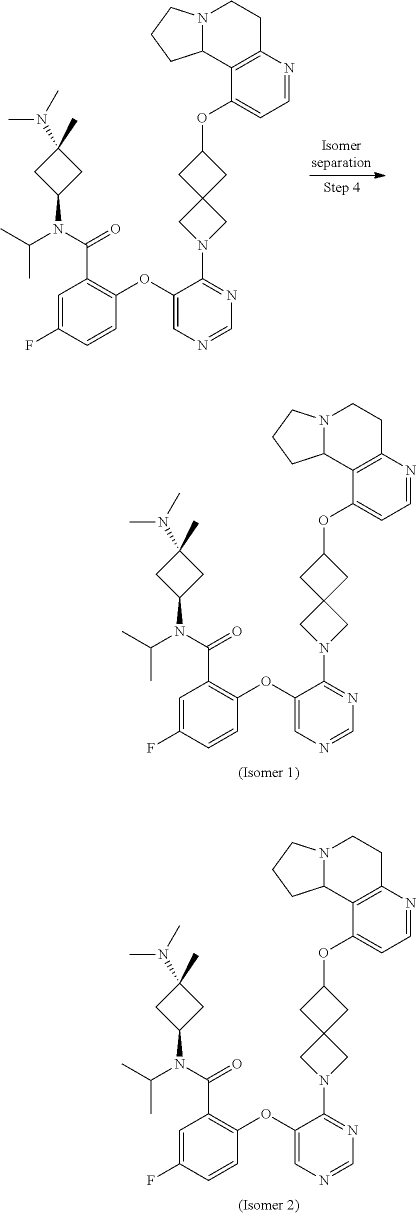 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(N(C)C)C1 ; CC(C)N(C(=O)c1cc(F)ccc1Oc1cncnc1N1CC2(CC(Oc3ccnc4c3C3CCCN3CC4)C2)C1)[C@H]1C[C@](C)(N(C)C)C1 ; CC(C)N(C(=O)c1cc(F)ccc1Oc1cncnc1N1CC2(CC(Oc3ccnc4c3C3CCCN3CC4)C2)C1)[C@H]1C[C@](C)(N(C)C)C1]

### Step 1. tert-Butyl ((1r,3r)-3-(5-fluoro-2-((4-(6-((5,6,8,9,10,10α-hexahydropyrrolo[2,1-f[[1,6]naphthyridin-1-yl)oxy)-2-azaspiro[3.3]heptan-2-yl)pyrimidin-5-yl)oxy)-N-isopropylbenzamido)-1-methylcyclobutyl)carbamate

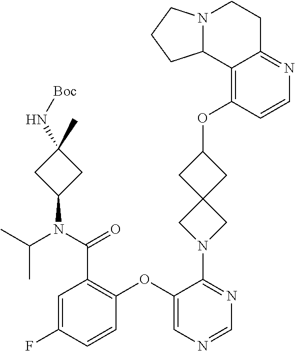 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(NC(=O)OC(C)(C)C)C1]

This compound was synthesized following the procedure described for the synthesis of Example 1, step 2, starting with 1 equivalent of (+)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoic acid, except that 1.5 equivalents of tert-butyl ((1r,3r)-3-(isopropylamino)-1-methylcyclobutyl)carbamate was used. The reaction was concentrated under reduced pressure. The crude was purified reverse phase chromatography using a SiliCycle SiliaSep C18 column (120 g) on Biotage isolera one (Mobile phase A: 10 mM NH₄HCO₃ in water, B: ACN) to obtain tert-butyl ((1r,3r)-3-(5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamido)-1-methylcyclobutyl)carbamate as a sticky solid.

Yield: 12.0%; LCMS (Method A): Rt=2.06 min, m/z=742.5 [M+H]⁺, 80.94%.

### Step 2. N-((1r,3r)-3-Amino-3-methylcyclobutyl)-5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropylbenz amide xHCl

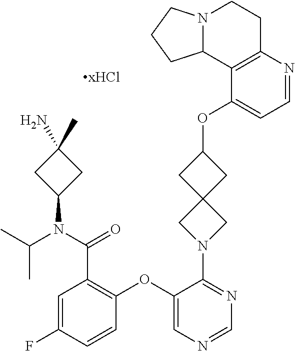 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(N)C1]

This compound was synthesized following the procedure described for the synthesis of Example 20, Step 3, starting with 1 equivalent of tert-butyl ((1r,3r)-3-(5-fluoro-2-((4-(6-((1,2,3,5,6,10b-hexahydropyrrolo[2,1-a]isoquinolin-10-yl)oxy)-2-azaspiro[3.3]heptan-2-yl)pyrimidin-5-yl)oxy)-N-isopropylbenzamido)-1-methylcyclobutyl)carbamate. After completion, the reaction was concentrated under reduced pressure. The residue was reconcentrated from ethyl acetate and dried under vacuum to obtain crude N-((1r,3r)-3-amino-3-methylcyclobutyl)-5-fluoro-2-((4-(6-((1,2,3,5,6,10b-hexahydropyrrolo[2,1-a]isoquinolin-10-yl)oxy)-2-azaspiro[3.3]heptan-2-yl)pyrimidin-5-yl)oxy)-N-isopropylbenzamide xHCl as a solid. This material was used without further purification.

Yield: 87%; LCMS (Method A): Rt=1.52 min, m/z=642.2 [M+H]⁺, 84.79%.

### Step 3. N-((1r,3r)-3-(Dimethylamino)-3-methylcyclobutyl)-5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropylbenzamide

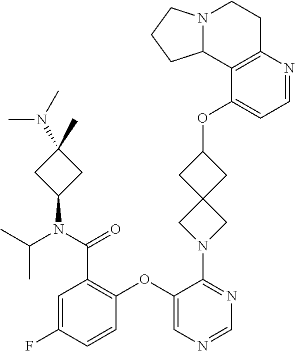 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(N(C)C)C1]

This compound was synthesized following the procedure described for the synthesis of Example 20, Step 4, starting with 1 equivalent of N-((1r,3r)-3-amino-3-methylcyclobutyl)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamide xHCl, except that 0.5 equivalent of AcOH and 2 equivalents of STAB were used. After completion, the reaction was concentrated under reduced pressure. The crude was purified by reverse phase chromatography using a SiliCycle SiliaSep C18 column (60 g) on Biotage isolera one (Mobile phase A: 10 mM NH₄HCO₃ in water, B: ACN). The pure fractions were concentrated under reduced pressure to afford N-((1r,3r)-3-(dimethylamino)-3-methylcyclobutyl)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamide as a solid.

Yield: 60.5%; LCMS (Method B): Rt=1.01 min, m/z=670.4 [M+H]⁺; HPLC (Method A): Rt=4.88 min, 90.79%; SFC (Method X). Peak 1: Rt=4.09 min, 49.35%; Peak 2: Rt=6.59 min, 47.21%.

### Step 4. (D1)-N-((1r,3r)-3-(Dimethylamino)-3-methylcyclobutyl)-5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropylbenzamide and (D2)-N-((1r,3r)-3-(dimethylamino)-3-methylcyclobutyl)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamide

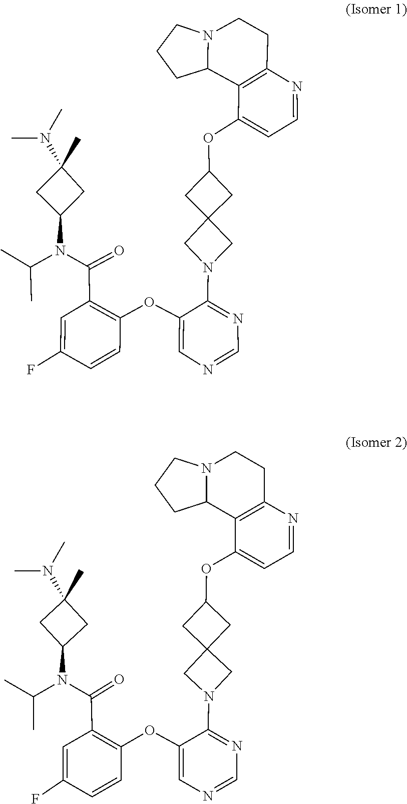 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(N(C)C)C1 ; CC(C)N(C(=O)c1cc(F)ccc1Oc1cncnc1N1CC2(CC(Oc3ccnc4c3C3CCCN3CC4)C2)C1)[C@H]1C[C@](C)(N(C)C)C1]

N-((1r,3r)-3-(Dimethylamino)-3-methylcyclobutyl)-5-fluoro-2-((4-(6-((1,2,3,5,6,1Ob-hexahydropyrrolo[2,1-a]isoquinolin-10-yl)oxy)-2-azaspiro[3.3]heptan-2-yl)pyrimidin-5-yl)oxy)-N-isopropylbenzamide (48 mg, 0.072 mmol) was purified by Chiral Prep SFC (Method E) to obtain (D1)-N-((1r,3r)-3-(dimethylamino)-3-methylcyclobutyl)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamide (10 mg, 20.67% yield) and (D2)-N-((1r,3r)-3-(dimethylamino)-3-methylcyclobutyl)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamide (7 mg, 14.44% yield) as solids.

Isomer 1: ¹H NMR (400 MHz, DMSO-d₆): δ 8.31-8.23 (m, 1H), 8.17 (br d, J=5.5 Hz, 1H), 7.78-7.57 (m, 1H), 7.32-7.06 (m, 3H), 6.70-6.61 (m, 1H), 4.75 (quin, J=6.3 Hz, 1H), 4.36-4.10 (m, 4H), 4.06-3.75 (m, 1H), 3.73-3.59 (m, 2H), 3.01-2.72 (m, 6H), 2.66-2.60 (m, 2H), 2.43-2.37 (m, 1H), 2.30-2.19 (m, 2H), 2.16-1.91 (m, 8H), 1.85-1.68 (m, 3H), 1.52-1.27 (m, 4H), 1.18-0.88 (m, 7H); LCMS (Method A): Rt=1.79 min, m/z=670.2 [M+H]⁺; HPLC (Method A): Rt=4.90 min, 99.37%; Chiral SFC (Method AJ)=Rt=3.19 min, 100%.

Isomer 2. ¹H NMR (400 MHz, DMSO-d₆): δ 8.31-8.23 (m, 1H), 8.17 (br d, J=5.5 Hz, 1H), 7.78-7.57 (m, 1H), 7.32-7.06 (m, 3H), 6.70-6.61 (m, 1H), 4.75 (quin, J=6.3 Hz, 1H), 4.36-4.10 (m, 4H), 4.06-3.75 (m, 1H), 3.73-3.59 (m, 2H), 3.01-2.72 (m, 6H), 2.66-2.60 (m, 2H), 2.43-2.37 (m, 1H), 2.30-2.19 (m, 2H), 2.15-1.95 (m, 8H), 1.85-1.68 (m, 3H), 1.52-1.27 (m, 4H), 1.18-0.88 (m, 7H); LCMS (Method A): Rt=1.82 min, m/z=670.2 [M+H]⁺; HPLC (Method A): Rt=4.89 min, 99.20%; Chiral SFC (Method AJ)=Rt=4.94 min, 98.98%.

### Example 59. (S)-5-Fluoro-N-isopropyl-N-(1-methylpyrrolidin-3-yl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide (Compound No. 59)

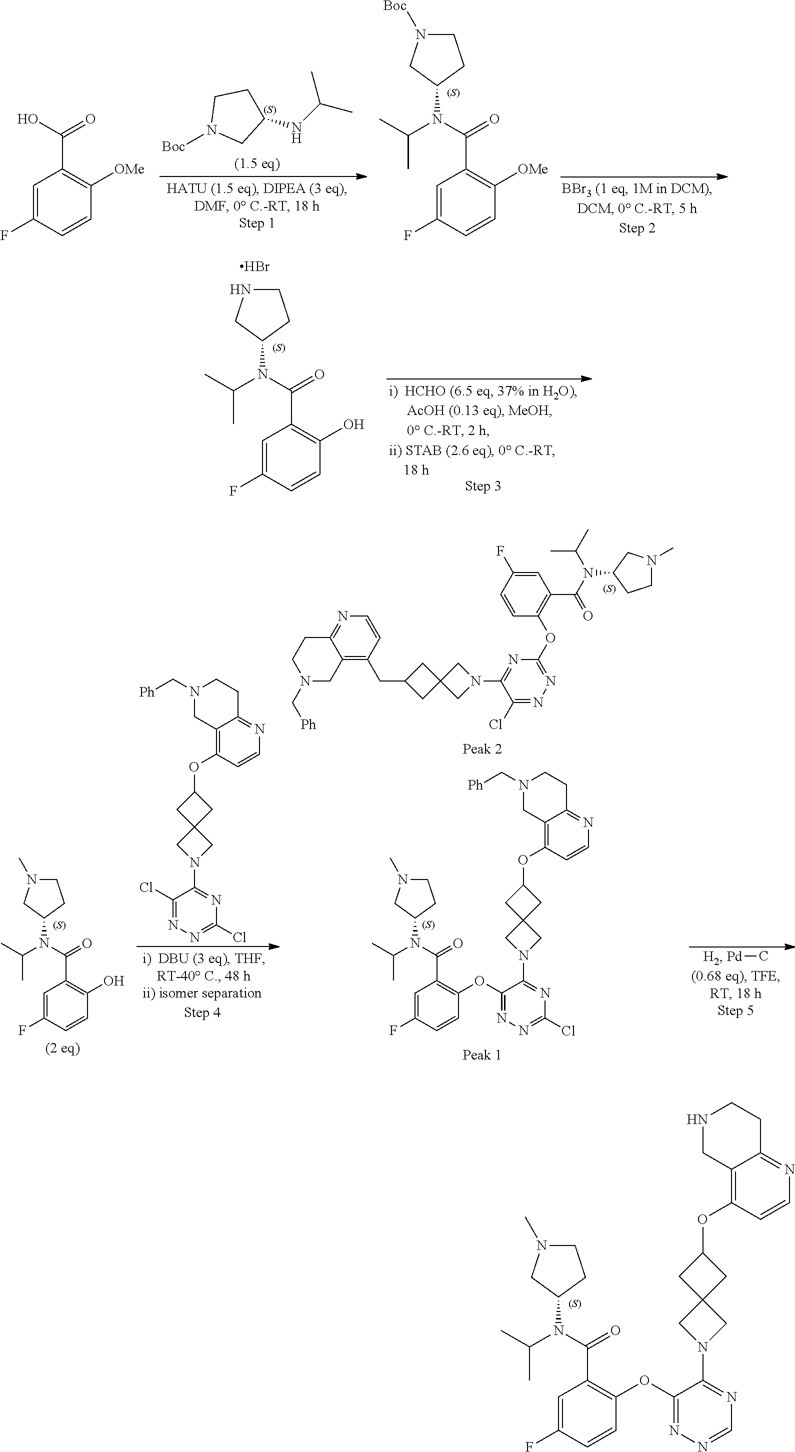

### Step 1. tert-Butyl (S)-3-(5-fluoro-N-isopropyl-2-methoxybenzamido)pyrrolidine-1-carboxylate

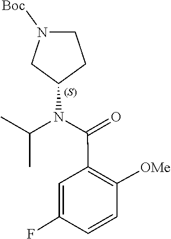

This compound was synthesized following the procedure described for the synthesis of Example 1, step 2, starting with 1 equivalent of 5-fluoro-2-methoxybenzoic acid and 1.5 equivalents of tert-butyl (S)-3-(isopropylamino)pyrrolidine-1-carboxylate. After work-up, the crude was purified by silica gel flash column chromatography using EtOAc in hexane (product eluted at 80% EtOAc in hexane) to afford tert-butyl (S)-3-(5-fluoro-N-isopropyl-2-methoxybenzamido)pyrrolidine-1-carboxylate as a sticky solid.

Yield: 61.32%; ¹H NMR (400 MHz, DMSO-d₆): δ 7.23-7.15 (m, 1H), 7.14-7.03 (m, 2H), 4.07-3.97 (m, 1H), 3.80-3.72 (m, 3H), 3.57-3.48 (m, 2H), 3.42-3.37 (m, 1H), 2.63-2.55 (m, 1H), 1.97-1.83 (m, 1H), 1.42 (s, 9H), 1.39-1.00 (m, 6H), two protons merged with solvent peaks; LCMS (Method A): Rt=2.07 min, m/z=381.1 [M+H]⁺, 79.96%.

### Step 2. (S)—5-Fluoro-2-hydroxy-N-isopropyl-N-(pyrrolidin-3-yl)benzamide hydrobromide

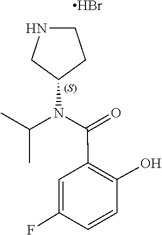

To a stirred solution of tert-butyl (S)-3-(5-fluoro-N-isopropyl-2-methoxybenzamido)pyrrolidine-1-carboxylate (2.5 g, 6.57 mmol) in DCM (70 mL), BBr₃ (6.57 mL, 6.57 mmol, 1 M in DCM) was added at 0° C. The reaction was stirred at RT for 5 h under a nitrogen atmosphere. The reaction progress was monitored by TLC (5% MeOH in DCM). After completion, the reaction was quenched with MeOH (20 mL) at 0° C. and concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure to obtain (S)-5-fluoro-2-hydroxy-N-isopropyl-N-(pyrrolidin-3-yl)benzamide hydrobromide (2.5 g, 73.63% yield) as a solid. ¹H NMR (400 MHz, DMSO-d₆): δ 9.80 (s, 1H), 8.92 (br s, 1H), 8.60-8.41 (m, 1H), 7.09-7.06 (m, 2H), 6.91-6.85 (m, 1H), 4.28-4.16 (m, 1H), 3.69-3.46 (m, 3H), 3.35-3.27 (m, 1H), 3.14-3.03 (m, 1H), 2.31-2.22 (m, 2H), 1.16-1.03 (m, 6H); LCMS (Method A): Rt=1.36 min, m/z=267.1 [M+H]⁺, 67.12%.

### Step 3. (S)-5-Fluoro-2-hydroxy-N-isopropyl-N-(1-methylpyrrolidin-3-yl)benzamide

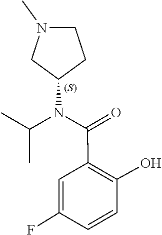

To a stirred solution of (S)-5-fluoro-2-hydroxy-N-isopropyl-N-(pyrrolidin-3-yl)benzamide hydrobromide (2.5 g, 7.22 mmol) in MeOH (40 mL), formaldehyde (3.49 mL, 46.9 mmol, 37% in H₂O) and AcOH (0.054 mL, 0.939 mmol) were added at 0° C. under a nitrogen atmosphere. The reaction was stirred at RT for 2 h. To this reaction mixture, STAB (3.98 g, 18.77 mmol) was added at 0° C. The reaction was stirred at RT for 18 h. The reaction was monitored by TLC (10% MeOH in DCM). After completion, the reaction was concentrated under reduced pressure. The crude product was purified by reverse phase chromatography using a SiliCycle SiliaSep C18 column (120 g) on Biotage isolera one (Mobile phase A: 10 mM NH₄HCO₃ in water, B: ACN). The pure fractions were concentrated under reduced pressure to obtain (S)-5-fluoro-2-hydroxy-N-isopropyl-N-(1-methylpyrrolidin-3-yl)benzamide (2.2 g, 69.0% yield) as a sticky liquid. LCMS (Method A): Rt=1.63 min, m/z=281.1 [M+H]⁺, 82.49%.

### Step 4. (S)-2-((5-(6-((6-Benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-(1-methylpyrrolidin-3-yl)benzamide and (S)-2-((5-(6-((6-benzyl-5,6,7,8-tetrahydro-1,6-naphthyridin-4-yl)oxy)-2-azaspiro[3.3]heptan-2-yl)-6-chloro-1,2,4-triazin-3-yl)oxy)-5-fluoro-N-isopropyl-N-(1-methylpyrrolidin-3-yl)benzamide

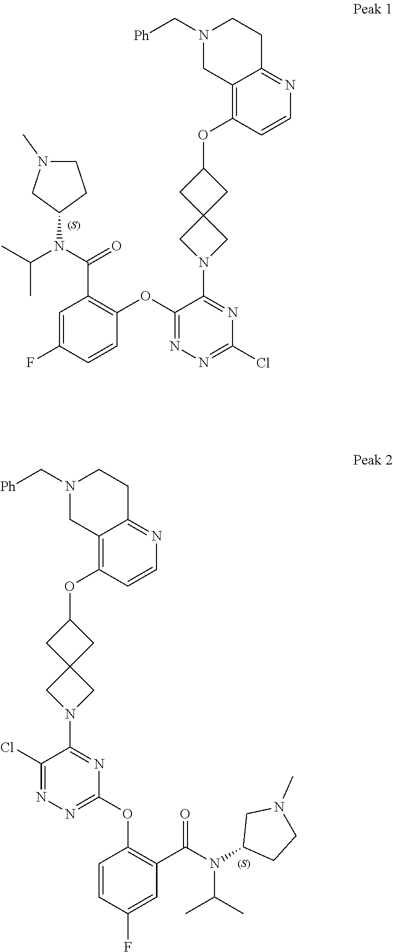

To a stirred solution of 6-benzyl-4-((2-(3,6-dichloro-1,2,4-triazin-5-yl)-2-azaspiro[3.3]heptan-6-yl)oxy)-5,6,7,8-tetrahydro-1,6-naphthyridine (1.9 g, 3.93 mmol) and (S)-5-fluoro-2-hydroxy-N-isopropyl-N-(1-methylpyrrolidin-3-yl)benzamide (2.204 g, 7.86 mmol) in THF (38 mL), DBU (1.777 mL, 11.79 mmol) was added at RT. The reaction was stirred at 40° C. for 48 h. The reaction was monitored by LCMS. After completion, the reaction was quenched with water (50 mL) and the mixture was extracted with EtOAc (3×60 mL). The combined organic layer was dried over sodium sulfate and filtered, and the filtrate was concentrated under reduced pressure. The crude was purified twice by silica gel flash column chromatography using MeOH—in DCM (product eluted at 20% MeOH in DCM) to obtain both regioisomers.

Peak 1: (S)-2-((5-(6-((6-Benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-(1-methylpyrrolidin-3-yl)benzamide (350 mg, 11.28% yield) as a solid. ¹H NMR (400 MHz, DMSO-d₆): δ 8.24-8.18 (m, 1H), 7.50-7.20 (m, 8H), 6.71-6.63 (m, 1H), 4.82-4.54 (m, 3H), 4.31-4.14 (m, 2H), 3.99-3.84 (m, 1H), 3.74-3.56 (m, 3H), 3.47 (s, 2H), 2.89-2.76 (m, 4H), 2.74-2.69 (m, 2H), 2.32-2.09 (m, 5H), 2.07-1.68 (m, 2H), 1.48-0.73 (m, 6H), four protons merged with solvent peaks: LCMS (Method A): Rt=2.15 min, m/z=727.2 [M+H]⁺, 93.12%.

Peak 2: (S)-2-((5-(6-((6-Benzyl-5,6,7,8-tetrahydro-1,6-naphthyridin-4-yl)oxy)-2-azaspiro[3.3]heptan-2-yl)-6-chloro-1,2,4-triazin-3-yl)oxy)-5-fluoro-N-isopropyl-N-(1-methylpyrrolidin-3-yl)benzamide (410 mg, 14.26% yield) as a solid. ¹H NMR (400 MHz, DMSO-d₆): δ 8.20 (br d, J=5.5 Hz, 1H), 7.40-7.16 (m, 8H), 6.67 (br d, J=5.5 Hz, 1H), 4.81-4.61 (m, 3H), 4.17-3.95 (m, 2H), 3.89-3.59 (m, 4H), 3.45 (s, 2H), 2.83-2.66 (m, 6H), 2.31-2.13 (m, 5H), 1.97-1.57 (m, 2H), 1.47-0.92 (m, 6H), four protons merged with solvent peaks; LCMS (Method A): Rt=2.17 min, m/z=727.3 [M+H]⁺, 99.43%.

### Step 5. (S)—5-Fluoro-N-isopropyl-N-(1-methylpyrrolidin-3-yl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide

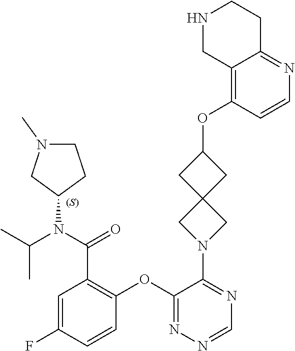

To a stirred solution of (S)-2-((5-(6-((6-benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-(1-methylpyrrolidin-3-yl)benzamide (77 mg, 0.106 mmol) in TFE (5 mL), Pd—C (77 mg, 0.072 mmol) was added at RT. The reaction was stirred at RT for 18 h under a hydrogen atmosphere (balloon pressure). The reaction progress was monitored by TLC (10% MeOH in DCM). After completion, the reaction was filtered through a Celite® pad, and the pad was washed with 2,2,2-trifluoroethanol (20 mL). The filtrate was concentrated under reduced pressure. The crude product was purified by reverse phase chromatography using a SiliCycle SiliaSep C18 column (40 g) on Biotage isolera one (Mobile phase A: 10 mM NH₄HCO₃ in water, B: ACN) to obtain (S)-5-fluoro-N-isopropyl-N-(1-methylpyrrolidin-3-yl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide (18 mg, 28.2% yield) as a solid. ¹HNMR (400 MHz, DMSO-d₆): δ 8.50-8.44 (m, 1H), 8.17 (d, J=5.5 Hz, 1H), 7.48-7.41 (m, 1H), 7.39-7.20 (m, 2H), 6.69-6.60 (m, 1H), 4.84-4.74 (m, 1H), 4.72-4.50 (m, 2H), 4.31-4.08 (m, 2H), 4.01-3.88 (m, 1H), 3.83-3.73 (m, 1H), 3.71 (s, 2H), 3.62-3.54 (m, 1H), 2.95 (br t, J=5.5 Hz, 2H), 2.89-2.75 (m, 2H), 2.71-2.65 (m, 3H), 2.31-2.03 (m, 5H), 2.01-1.80 (m, 2H), 1.48-0.58 (m, 6H), three protons merged with solvent peaks; LCMS (Method C): Rt=1.47 min, m/z=603.2 [M+H]⁺; HPLC (Method A): Rt=3.79 min, 97.70%; Chiral SFC (Method J): Rt=6.47 min, 100%.

### Example 60. 5-Fluoro-N-isopropyl-2-((5-(6-((6-methyl-5,6,7,8-tetrahydro-1,6-naphthyridin-4-yl)oxy)-2-azaspiro[3.3]heptan-2-yl)-1,2,4-triazin-6-yl)oxy)-N-(1-methylpiperidin-4-yl)benzamide (Compound No. 60)

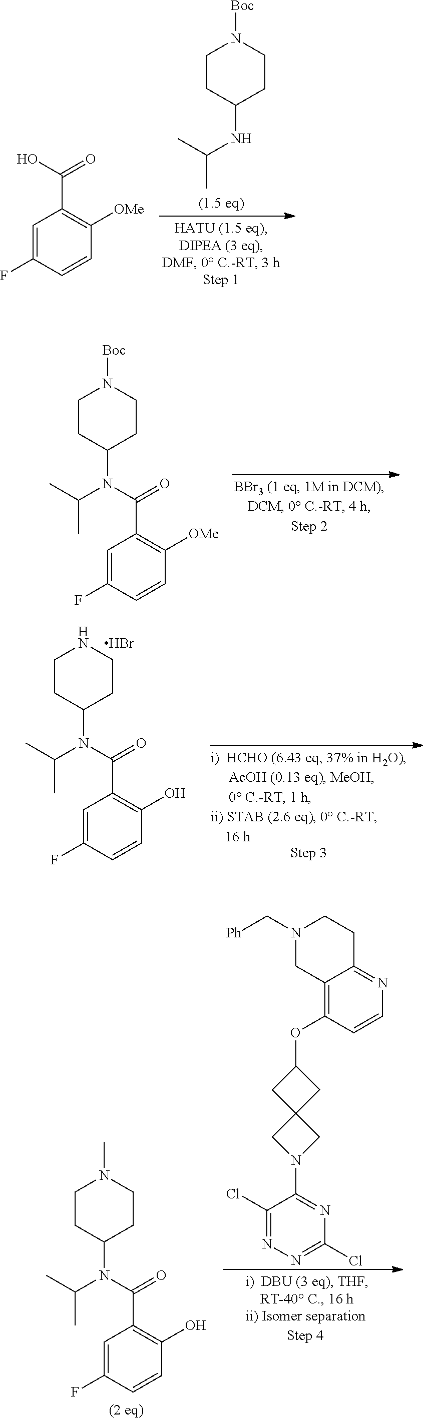

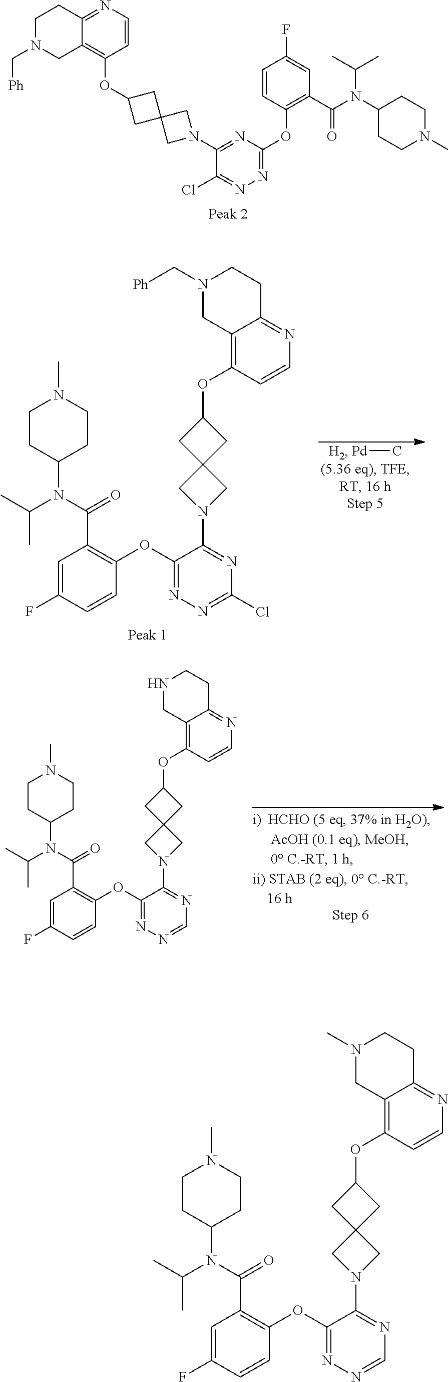

### Step 1. tert-Butyl 4-(5-fluoro-N-isopropyl-2-methoxybenzamido)piperidine-1-carboxylate

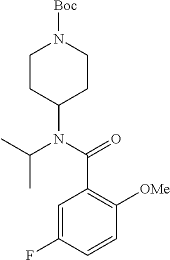 [SMILES: COc1ccc(F)cc1C(=O)N(C(C)C)C1CCN(C(=O)OC(C)(C)C)CC1]

This compound was synthesized following the procedure described for the synthesis of Example 1, step 2, starting with 1 equivalent of 5-fluoro-2-methoxybenzoic acid and 1.5 equivalents of tert-butyl 4-(isopropylamino)piperidine-1-carboxylate. After work-up, the crude was purified by silica gel flash column chromatography using EtOAc in hexane (product eluted at 80% EtOAc in hexane) to afford tert-butyl 4-(5-fluoro-N-isopropyl-2-methoxybenzamido)piperidine-1-carboxylate as a solid.

Yield: 45.3%; LCMS (Method A): Rt=2.15 min, m/z=339.2 [M+H−56]⁺, 70.48%.

### Step 2. 5-Fluoro-2-hydroxy-N-isopropyl-N-(piperidin-4-yl)benzamide hydrobromide

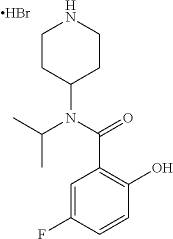 [SMILES: Br ; CC(C)N(C(=O)c1cc(F)ccc1O)C1CCNCC1]

This compound was synthesized following the procedure described for the synthesis of Example 59, step 2, starting with 1 equivalent of tert-butyl 4-(5-fluoro-N-isopropyl-2-methoxybenzamido)piperidine-1-carboxylate. After completion, the reaction was quenched with MeOH at 0° C., and the mixture was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure to obtain 5-fluoro-2-hydroxy-N-isopropyl-N-(piperidin-4-yl)benzamide hydrobromide as a liquid.

Yield: 49.37%; ¹H NMR (400 MHz, DMSO-d₆): δ 9.79-9.53 (m, 1H), 8.24-7.77 (m, 2H), 7.02 (dt, J=3.2, 8.7 Hz, 1H), 6.91 (dd, J=3.0, 8.5 Hz, 1H), 6.85 (dd, J=4.6, 8.9 Hz, 1H), 3.71-3.60 (m, 1H), 3.50-3.38 (m, 1H), 3.04-2.81 (m, 3H), 1.79-1.55 (m, 2H), 1.48-1.04 (m, 6H), three protons merged with solvent peaks; LCMS (Method A): Rt=1.23 min, m/z=281.0 [M+H]⁺, 63.45%.

### Step 3. 5-Fluoro-2-hydroxy-N-isopropyl-N-(1-methylpiperidin-4-yl)benzamide

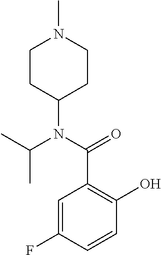 [SMILES: CC(C)N(C(=O)c1cc(F)ccc1O)C1CCN(C)CC1]

This compound was synthesized following the procedure described for the synthesis of Example 59, step 3, starting with 1 equivalent 5-fluoro-2-hydroxy-N-isopropyl-N-(piperidin-4-yl)benzamide hydrobromide, except that 6.43 equivalents of formaldehyde (37% in H₂O) was used. After completion, the reaction was concentrated under reduced pressure. The crude product was purified by reverse phase chromatography using a SiliCycle SiliaSep C18 column (120 g) on Biotage isolera one (Mobile phase A: 10 mM NH₄HCO₃ in water, B: ACN). The pure fractions were concentrated under reduced pressure to obtain 5-fluoro-2-hydroxy-N-isopropyl-N-(1-methylpiperidin-4-yl)benzamide as a liquid.

Yield: 65.7%; LCMS (Method A): Rt=1.42 min, m/z=295.1 [M+H]⁺, 80.46%.

### Step 4. 2-((5-(6-((6-Benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-(1-methylpiperidin-4-yl)benzamide and 2-((5-(6-((6-benzyl-5,6,7,8-tetrahydro-1,6-naphthyridin-4-yl)oxy)-2-azaspiro[3.3]heptan-2-yl)-6-chloro-1,2,4-triazin-3-yl)oxy)-5-fluoro-N-isopropyl-N-(1-methylpiperidin-4-yl)benzamide

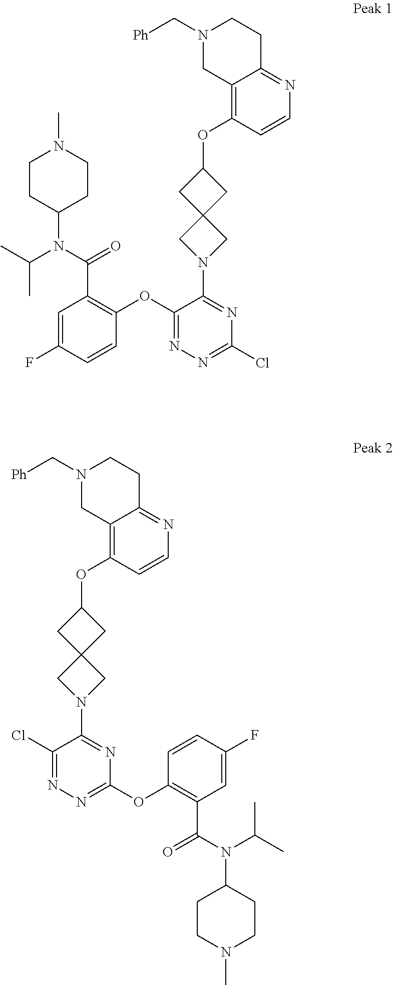 [SMILES: CC(C)N(C(=O)c1cc(F)ccc1Oc1nnc(Cl)nc1N1CC2(CC(Oc3ccnc4c3CN(Cc3ccccc3)CC4)C2)C1)C1CCN(C)CC1 ; CC(C)N(C(=O)c1cc(F)ccc1Oc1nnc(Cl)c(N2CC3(CC(Oc4ccnc5c4CN(Cc4ccccc4)CC5)C3)C2)n1)C1CCN(C)CC1]

This compound was synthesized following the procedure described for the synthesis of Example 59, step 4, starting with 1 equivalent of 6-benzyl-4-((2-(3,6-dichloro-1,2,4-triazin-5-yl)-2-azaspiro[3.3]heptan-6-yl)oxy)-5,6,7,8-tetrahydro-1,6-naphthyridine, except that 2 equivalents for 5-fluoro-2-hydroxy-N-isopropyl-N-(1-methylpiperidin-4-yl)benzamide was used. After work-up, the crude was purified by silica gel flash column chromatography using 20% MeOH in DCM, then by reverse phase chromatography using a SiliCycle SiliaSep C18 column (40 g) on Biotage isolera one (Mobile phase A: 10 mM NH₄HCO₃ in water, B: ACN) to obtain desired isomer.

Desired isomer: 2-((5-(6-((6-benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-(1-methylpiperidin-4-yl)benzamide as a solid. Yield: 20.18%; IH NMR (400 MHz, DMSO-d₆): δ 8.24-8.17 (m, 1H), 7.50-7.25 (m, 8H), 6.71-6.62 (m, 1H), 4.83-4.72 (m, 1H), 4.69-4.57 (m, 2H), 4.30-4.17 (m, 2H), 3.75-3.63 (m, 2H), 3.52-3.43 (m, 2H), 3.04-2.95 (m, 1H), 2.87-2.67 (m, 8H), 2.35-2.10 (m, 4H), 2.07-1.95 (m, 2H), 1.70-1.50 (m, 2H), 1.46-1.20 (m, 5H), 1.13-0.64 (m, 5H); LCMS (Method A): Rt=2.20 min, m/z=741.4 [M+H]⁺, 98.42%.

Note: Undesired isomer was not isolated

### Step 5. 5-Fluoro-N-isopropyl-N-(1-methylpiperidin-4-yl)-2-((S-(6-((5,6,7,8-tetrahydro-1,6-naphthyridin-4-yl)oxy)-2-azaspiro[3.3]heptan-2-yl)-1,2,4-triazin-6-yl)oxy)benzamide

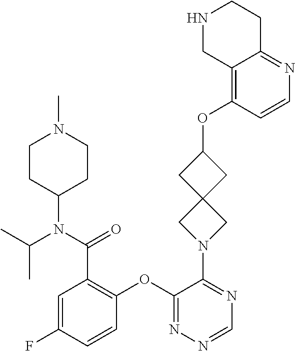 [SMILES: CC(C)N(C(=O)c1cc(F)ccc1Oc1nncnc1N1CC2(CC(Oc3ccnc4c3CNCC4)C2)C1)C1CCN(C)CC1]

This compound was synthesized following the procedure described for the synthesis of Example 59, step 5, starting with 1 equivalent of 2-((5-(6-((6-benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-(1-methylpiperidin-4-yl)benzamide, except that 0.54 equivalent of Pd-C was used. After completion, the reaction was filtered through a Celite® pad, and the pad was washed with MeOH. The filtrate was concentrated under reduced pressure to obtain crude 5-fluoro-N-isopropyl-N-(1-methylpiperidin-4-yl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide as solid. This material was used without further purification.

Yield: 57.7%; LCMS (Method C): Rt=1.34 min, m/z=617.3 [M+H]⁺, 96.65%.

### Step 6. 5-Fluoro-N-isopropyl-2-((S-(6-((6-methyl-5,6,7,8-tetrahydro-1,6-naphthyridin-4-yl)oxy)-2-azaspiro[3.3]heptan-2-yl)-1,2,4-triazin-6-yl)oxy)-N-(1-methylpiperidin-4-yl)benzamide

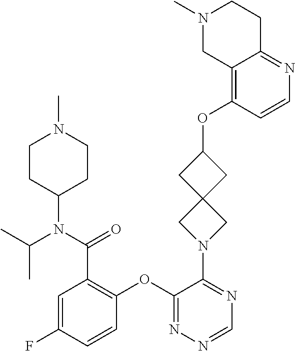 [SMILES: CC(C)N(C(=O)c1cc(F)ccc1Oc1nncnc1N1CC2(CC(Oc3ccnc4c3CN(C)CC4)C2)C1)C1CCN(C)CC1]

To a stirred solution of 5-fluoro-N-isopropyl-N-(1-methylpiperidin-4-yl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide (60 mg, 0.097 mmol) in MeOH (1.5 mL), formaldehyde (0.048 mL, 0.486 mmol, 37% in H₂O) and AcOH (0.556 μL, 9.73 μmol) were added at 0° C. The reaction was stirred at RT for 1 h. To this reaction mixture, STAB (41.2 mg, 0.195 mmol) was added at 0° C. and the reaction was stirred at RT for 16 h. The reaction progress was monitored by TLC (10% MeOH in DCM). After completion, the reaction was concentrated under reduced pressure, and saturated aqueous sodium bicarbonate solution (10 mL) was added. The mixture was extracted with 10% MeOH in DCM (3×10 mL). The combined organic layer was dried over anhydrous sodium sulfate and filtered, and the filtrate was concentrated under reduced pressure. The crude compound was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to obtain 5-fluoro-N-isopropyl-2-((5-(6-((6-methyl-5,6,7,8-tetrahydro-1,6-naphthyridin-4-yl)oxy)-2-azaspiro[3.3]heptan-2-yl)-1,2,4-triazin-6-yl)oxy)-N-(1-methylpiperidin-4-yl)benzamide (15.36 mg, 24.93% yield) as a solid. ¹HNMR (400 MHz, DMSO-d₆): δ 8.49-8.40 (m, 1H), 8.20 (d, J=5.4 Hz, 1H), 7.48-7.41 (m, 1H), 7.39-7.28 (m, 2H), 6.68 (d, 0.1=5.6 Hz, 1H), 4.85-4.74 (m, 1H), 4.69-4.52 (m, 2H), 4.29-4.08 (m, 2H), 3.38 (br s, 2H), 3.10-2.90 (m, 1H), 2.89-2.76 (m, 5H), 2.72-2.61 (m, 3H), 2.58-2.54 (m, 2H), 2.37 (br s, 3H), 2.19-2.12 (m, 3H), 1.96-1.84 (m, 1H), 1.70-1.46 (m, 5H), 1.33-0.49 (m, 6H), one proton merged with solvent peaks; LCMS (Method A): Rt=1.57 min, m/z=631.2 [M+H]⁺; HPLC (Method A): Rt=4.39 min, 99.82%.

### Example 61. N-((1r,3r)-3-(Dimethylamino)-3-methylcyclobutyl)-5-fluoro-N-isopropyl-2-((5-(6-((6-methyl-5,6,7,8-tetrahydro-1,6-naphthyridin-4-yl)oxy)-2-azaspiro[3.3]heptan-2-yl)-1,2,4-triazin-6-yl)oxy)benzamide (Compound No. 61)

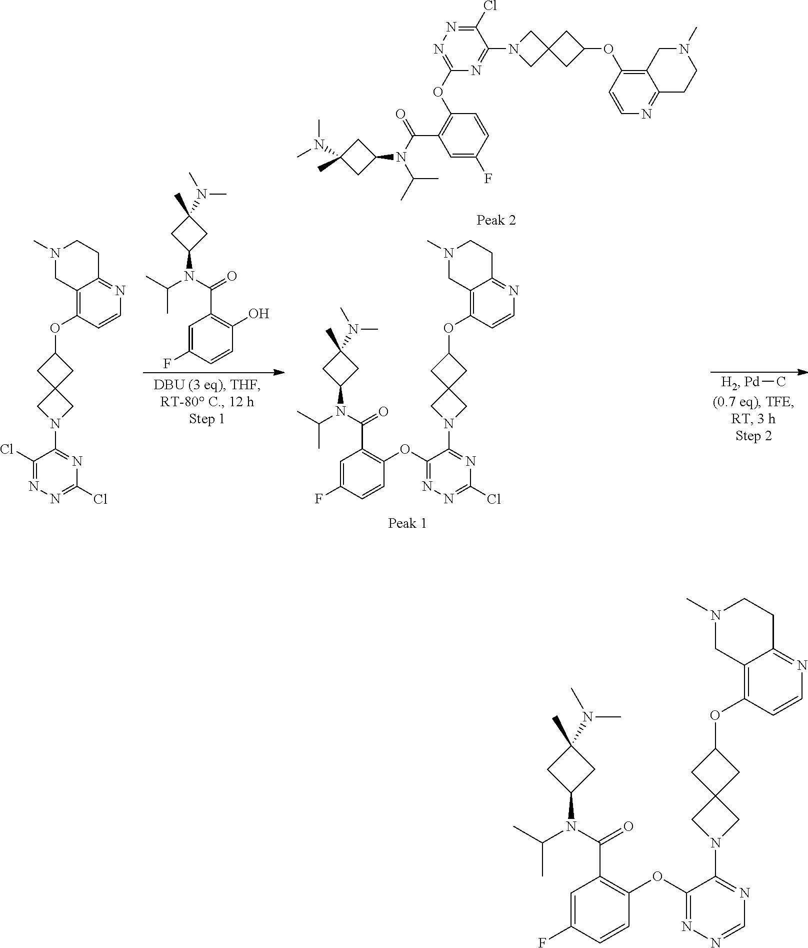

### Step 1. 2-((3-Chloro-5-(6-((6-methyl-5,6,7,8-tetrahydro-1,6-naphthyridin-4-yl)oxy)-2-azaspiro[3.3]heptan-2-yl)-1,2,4-triazin-6-yl)oxy)-N-((1r,3r)-3-(dimethylamino)-3-methylcyclobutyl)-5-fluoro-N-isopropylbenzamide and 2-((6-chloro-5-(6-((6-methyl-5,6,7,8-tetrahydro-1,6-naphthyridin-4-yl)oxy)-2-azaspiro[3.3]heptan-2-yl)-1,2,4-triazin-3-yl)oxy)-N-((1r,3r)-3-(dimethylamino)-3-methylcyclobutyl)-5-fluoro-N-isopropylbenzamide

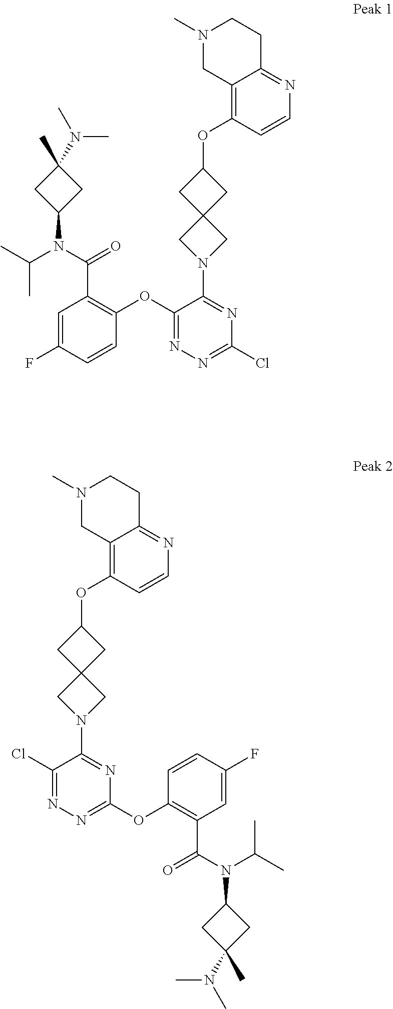 [SMILES: CC(C)N(C(=O)c1cc(F)ccc1Oc1nnc(Cl)nc1N1CC2(CC(Oc3ccnc4c3CN(C)CC4)C2)C1)[C@H]1C[C@](C)(N(C)C)C1 ; CC(C)N(C(=O)c1cc(F)ccc1Oc1nnc(Cl)c(N2CC3(CC(Oc4ccnc5c4CN(C)CC5)C3)C2)n1)[C@H]1C[C@](C)(N(C)C)C1]

This compound was synthesized following the procedure described for the synthesis of Example 59, step 4, starting with 1 equivalent of 4-((2-(3,6-dichloro-1,2,4-triazin-5-yl)-2-azaspiro[3.3]heptan-6-yl)oxy)-6-methyl-5,6,7,8-tetrahydro-1,6-naphthyridine and 1 equivalent of N-((1r,3r)-3-(dimethylamino)-3-methylcyclobutyl)-5-fluoro-2-hydroxy-N-isopropylbenzamide. The reaction was stirred at 80° C. After workup, The crude was purified by reverse phase chromatography using a SiliCycle SiliaSep C18 column (12 g) on Biotage isolera one (Mobile phase A: 10 mM NH₄HCO₃ in water, B: ACN) to obtain 2-((3-chloro-5-(6-((6-methyl-5,6,7,8-tetrahydro-1,6-naphthyridin-4-yl)oxy)-2-azaspiro[3.3]heptan-2-yl)-1,2,4-triazin-6-yl)oxy)-N-((1r,3r)-3-(dimethylamino)-3-methylcyclobutyl)-5-fluoro-N-isopropylbenzamide and the other isomer (54.5% yield) as a solid. LCMS (Method A): Rt=1.92 min, m/z=679.4 [M+H]⁺, 94.40%; Chiral SFC (Method E): Peak-L: Rt=2.40 min, 60.46%; Peak-2: Rt=5.03 min, 35.67%.

This mixture was purified by Chiral SFC (Method H) to obtain Peak-1.

Peak-1. 2-((3-Chloro-5-(6-((6-methyl-5,6,7,8-tetrahydro-1,6-naphthyridin-4-yl)oxy)-2-azaspiro[3.3]heptan-2-yl)-1,2,4-triazin-6-yl)oxy)-N-((1r,3r)-3-(dimethylamino)-3-methylcyclobutyl)-5-fluoro-N-isopropylbenzamide (60% yield) as a solid. ¹H NMR (400 MHz, DMSO-d₆): δ 8.28-8.15 (m, 1H), 7.54-7.13 (m, 3H), 6.74-6.61 (m, 1H), 4.88-4.54 (m, 3H), 4.36-4.17 (m, 2H), 3.98-3.72 (m, 1H), 3.68-3.54 (m, 1H), 3.41-3.36 (m, 2H), 2.95-2.78 (m, 4H), 2.67-2.61 (m, 2H), 2.42-2.25 (m, 5H), 2.15-2.10 (m, 2H), 2.08 (s, 3H), 1.99-1.87 (m, 3H), 1.84-1.68 (m, 1H), 1.64-1.53 (m, 1H), 1.46-1.35 (m, 1H), 1.30-1.14 (m, 2H), 1.11-0.64 (m, 5H), one proton merged with solvent peaks; LCMS (Method A): Rt=1.90 min, m/z=679.1 [M+H]⁺, 97.57%; Chiral SFC (Method E): Rt=2.38 min, 100%.

Note. Peak-2 was not isolated.

### Step 2. N-((1r,3r)-3-(Dimethylamino)-3-methylcyclobutyl)-5-fluoro-N-isopropyl-2-((5-(6-((6-methyl-5,6,7,8-tetrahydro-1,6-naphthyridin-4-yl)oxy)-2-azaspiro[3.3]heptan-2-yl)-1,2,4-triazin-6-yl)oxy)benzamide

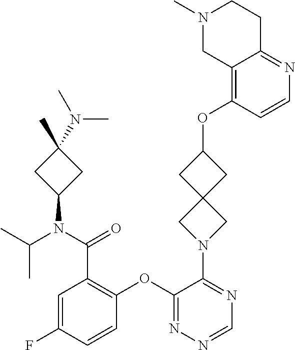 [SMILES: CC(C)N(C(=O)c1cc(F)ccc1Oc1nncnc1N1CC2(CC(Oc3ccnc4c3CN(C)CC4)C2)C1)[C@H]1C[C@](C)(N(C)C)C1]

This compound was synthesized following the procedure described for the synthesis of Example 59, step 4, starting with 1 equivalent of 2-((3-chloro-5-(6-((6-methyl-5,6,7,8-tetrahydro-1,6-naphthyridin-4-yl)oxy)-2-azaspiro[3.3]heptan-2-yl)-1,2,4-triazin-6-yl)oxy)-N-((1r,3r)-3-(dimethylamino)-3-methylcyclobutyl)-5-fluoro-N-isopropylbenzamide, except that 0.7 equivalent of Pd-C was used. After filtration and concentration, the crude product was purified by reverse phase chromatography using a SiliCycle SiliaSep C18 column (40 g) on Biotage isolera one (Mobile phase A: 10 mM NH₄HCO₃ in water, B: ACN) to afford N-((1r,3r)-3-(dimethylamino)-3-methylcyclobutyl)-5-fluoro-N-isopropyl-2-((5-(6-((6-methyl-5,6,7,8-tetrahydro-1,6-naphthyridin-4-yl)oxy)-2-azaspiro[3.3]heptan-2-yl)-1,2,4-triazin-6-yl)oxy)benzamide (63.8% yield) as a solid. ¹H NMR (400 MHz, DMSO-d₆): δ 8.46 (s, 1H), 8.19 (br d, J=5.3 Hz, 1H), 7.53-7.08 (m, 3H), 6.74-6.62 (m, 1H), 4.88-4.76 (m, 1H), 4.72-4.47 (m, 2H), 4.33-4.07 (m, 2H), 4.03-3.68 (m, 1H), 3.65-3.50 (m, 1H), 3.37 (s, 2H), 2.92-2.76 (m, 4H), 2.66-2.60 (m, 2H), 2.42-2.22 (m, 6H), 2.17-2.04 (m, 4H), 1.95 (br s, 3H), 1.83-1.66 (m, 1H), 1.59-1.48 (m, 1H), 1.53-1.46 (m, 9H); LCMS (Method A): Rt=1.68 min, m/z=645.2 [M+H]⁺; HPLC (Method A): Rt=4.75 min, 99.13%; Chiral SFC (Method E): Rt=2.75 min, 100%.

### Example 62. N-((1r,3r)-3-Aminocyclobutyl)-5-fluoro-N-isopropyl-2-((4-(6-((6-(2-methoxyethyl)-5,6,7,8-tetrahydro-1,6-naphthyridin-4-yl)oxy)-2-azaspiro[3.3]heptan-2-yl)pyrimidin-5-yl)oxy)benzamide (Compound No. 62)

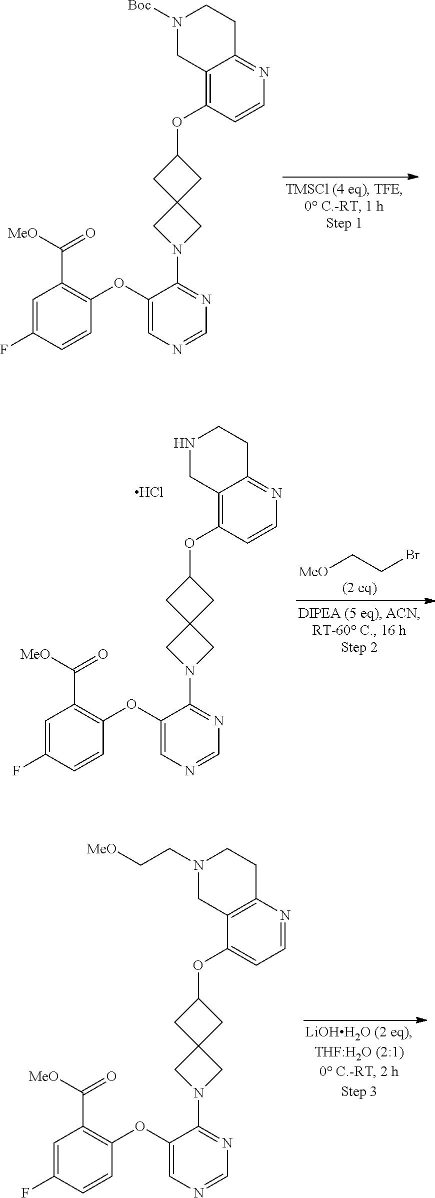 [SMILES: COC(=O)c1cc(F)ccc1Oc1cncnc1N1CC2(CC(Oc3ccnc4c3CN(C(=O)OC(C)(C)C)CC4)C2)C1 ; COC(=O)c1cc(F)ccc1Oc1cncnc1N1CC2(CC(Oc3ccnc4c3CNCC4)C2)C1 ; Cl ; COCCBr ; COCCN1CCc2nccc(OC3CC4(C3)CN(c3ncncc3Oc3ccc(F)cc3C(=O)OC)C4)c2C1]

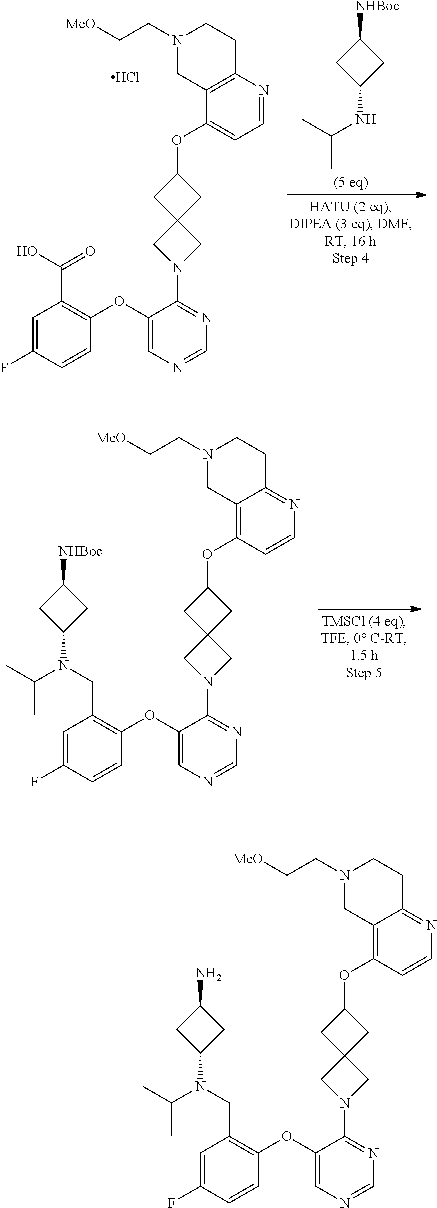

### Step 1. Methyl 5-fluoro-2-((4-(6-((5,6,7,8-tetrahydro-1,6-naphthyridin-4-yl)oxy)-2-azaspiro[3.3]heptan-2-yl)pyrimidin-5-yl)oxy)benzoate Hydrochloride

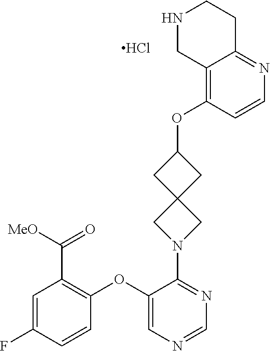 [SMILES: COC(=O)c1cc(F)ccc1Oc1cncnc1N1CC2(CC(Oc3ccnc4c3CNCC4)C2)C1 ; Cl]

This compound was synthesized following the procedure described for the synthesis of Example 20, step 3, starting with 1 equivalent of tert-butyl 4-((2-(5-(4-fluoro-2-(methoxycarbonyl)phenoxyl)pyrimidin-4-yl)-2-azaspiro[3.3]heptan-6-yl)oxy)-7,8-dihydro-1,6-naphthyridine-6(5H)-carboxylate. After completion, the reaction was concentrated under reduced pressure to obtain crude methyl 5-fluoro-2-((4-(6-((5,6,7,8-tetrahydro-1,6-naphthyridin-4-yl)oxy)-2-azaspiro[3.3]heptan-2-yl)pyrimidin-5-yl)oxy)benzoate hydrochloride as a solid. This material was used without further purification.

Yield: 98%; LCMS (Method A): Rt=1.54 min, m/z=492.2 [M+H]⁺, 98.75%.

### Step 2. Methyl 5-fluoro-2-((4-(6-((6-(2-methoxyethyl)-5,6,7,8-tetrahydro-1,6-naphthyridin-4-yl)oxy)-2-azaspiro[3.3]heptan-2-yl)pyrimidin-5-yl)oxy)benzoate

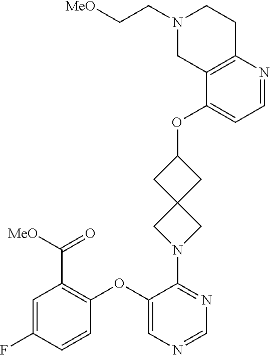 [SMILES: COCCN1CCc2nccc(OC3CC4(C3)CN(c3ncncc3Oc3ccc(F)cc3C(=O)OC)C4)c2C1]

Step 2 was performed following the same procedure in two batches (2×1 g).

To a stirred solution of methyl 5-fluoro-2-((4-(6-((5,6,7,8-tetrahydro-1,6-naphthyridin-4-yl)oxy)-2-azaspiro[3.3]heptan-2-yl)pyrimidin-5-yl)oxy)benzoate hydrochloride (1 g, 1.894 mmol) in acetonitrile (10 mL), DIPEA (1.654 mL, 9.47 mmol) was added at RT under a nitrogen atmosphere. To this reaction mixture, 1-bromo-2-methoxyethane (0.356 mL, 3.79 mmol) was added and the reaction was stirred at 60° C. for 16 h. The reaction progress was monitored by LCMS. After completion, the reaction mixtures from both batches were combined and diluted with water (50 mL). The mixture was extracted with EtOAc (3×40 mL). The combined organic layer was dried over anhydrous sodium sulfate and filtered, and the filtrate was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain methyl 5-fluoro-2-((4-(6-((6-(2-methoxyethyl)-5,6,7,8-tetrahydro-1,6-naphthyridin-4-yl)oxy)-2-azaspiro[3.3]heptan-2-yl)pyrimidin-5-yl)oxy)benzoate (1.034 g, combined yield) as a solid. ¹H NMR (400 MHz, DMSO-d₆): δ 8.29 (s, 1H), 8.18 (d, J=5.6 Hz, 1H), 7.69-7.65 (m, 2H), 7.53-7.43 (m, 1H), 7.11 (dd, J=4.5, 9.1 Hz, 1H), 6.67 (d, J=5.8 Hz, 1H), 4.75 (quin, J=6.6 Hz, 1H), 4.27 (s, 2H), 4.19 (s, 2H), 3.81 (s, 3H), 3.50 (t, J=5.8 Hz, 2H), 3.46 (br s, 2H), 3.25 (s, 3H), 2.83-2.73 (m, 6H), 2.67 (t, J=5.8 Hz, 2H), 2.31-2.23 (m, 2H); LCMS (Method A): Rt=1.71 min, m/z=550.1 [M+H]⁺, 98.79%.

### Step 3. 5-Fluoro-2-((4-(6-((6-(2-methoxyethyl)-5,6,7,8-tetrahydro-1,6-naphthyridin-4-yl)oxy)-2-azaspiro[3.3]heptan-2-yl)pyrimidin-5-yl)oxy)benzoic acid hydrochloride

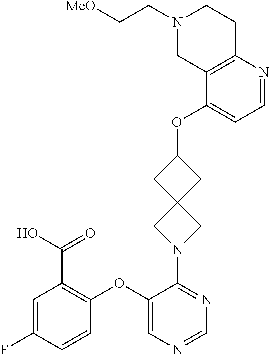 [SMILES: COCCN1CCc2nccc(OC3CC4(C3)CN(c3ncncc3Oc3ccc(F)cc3C(=O)O)C4)c2C1]

To a stirred solution of methyl 5-fluoro-2-((4-(6-((6-(2-methoxyethyl)-5,6,7,8-tetrahydro-1,6-naphthyridin-4-yl)oxy)-2-azaspiro[3.3]heptan-2-yl)pyrimidin-5-yl)oxy)benzoate (1 g, 1.819 mmol) in THF:H₂O (2:1, 12 mL), LiOH·H₂O (0.153 g, 3.64 mmol) was added at 0° C. The reaction was stirred at RT for 2 h. The reaction progress was monitored by TLC (10% MeOH in DCM). After completion, the reaction was concentrated under reduced pressure. The residue was acidified with 1.5 N HCl (pH˜5) and the mixture was concentrated under reduced pressure. The crude was re-concentrated from toluene (2×10 mL) and the residue was dried under reduced pressure to obtain crude 5-fluoro-2-((4-(6-((6-(2-methoxyethyl)-5,6,7,8-tetrahydro-1,6-naphthyridin-4-yl)oxy)-2-azaspiro[3.3]heptan-2-yl)pyrimidin-5-yl)oxy)benzoic acid hydrochloride (0.900 g, 91% yield) as a solid. This material was used without further purification. LCMS (Method A): Rt=1.22 min, m/z=536.2 [M+H]⁺, 98.53%.

### Step 4. tert-Butyl ((1r,3r)-3-(5-fluoro-N-isopropyl-2-((4-(6-((6-(2-methoxyethyl)-5,6,7,8-tetrahydro-1,6-naphthyridin-4-yl)oxy)-2-azaspiro[3.3]heptan-2-yl)pyrimidin-5-yl)oxy)benzamido)cyclobutyl)carbamate

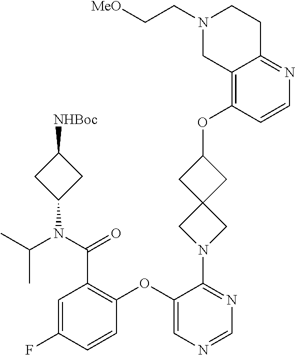 [SMILES: COCCN1CCc2nccc(OC3CC4(C3)CN(c3ncncc3Oc3ccc(F)cc3C(=O)N(C(C)C)[C@H]3C[C@H](NC(=O)OC(C)(C)C)C3)C4)c2C1]

This compound was synthesized following the procedure described for the synthesis of Example 1, step 2, starting with 1 equivalent of 5-fluoro-2-((4-(6-((6-(2-methoxyethyl)-5,6,7,8-tetrahydro-1,6-naphthyridin-4-yl)oxy)-2-azaspiro[3.3]heptan-2-yl)pyrimidin-5-yl)oxy)benzoic acid and 5 equivalents of tert-butyl ((1r,3r)-3-(isopropylamino)cyclobutyl)carbamate, except that 2 equivalents of HATU was used. The crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl ((1r,3r)-3-(5-fluoro-N-isopropyl-2-((4-(6-((6-(2-methoxyethyl)-5,6,7,8-tetrahydro-1,6-naphthyridin-4-yl)oxy)-2-azaspiro[3.3]heptan-2-yl)pyrimidin-5-yl)oxy)benzamido)cyclobutyl)carbamate as a solid.

Yield: 35.7%; ¹H NMR (400 MHz, DMSO-d₆): δ 8.30-8.26 (m, 1H), 8.18 (d, J=5.6 Hz, 1H), 7.83-7.73 (m, 1H), 7.32-7.21 (m, 2H), 7.15-7.03 (m, 1H), 6.66 (d, J=5.8 Hz, 1H), 4.80-4.68 (m, 11H), 4.41-4.06 (m, 6H), 3.82-3.63 (m, 2H), 3.51 (t, 0.1=5.8 Hz, 2H), 3.47 (br s, 2H), 3.26 (s, 3H), 2.83-2.74 (m, 6H), 2.68 (t, J=5.8 Hz, 2H), 2.31-2.21 (m, 2H), 2.11-1.83 (m, 2H), 1.46-1.26 (m, 12H), 1.07-0.92 (m, 3H), two protons merged with solvent peaks; LCMS (Method A): Rt=1.91 min, m/z=746.2 [M+H]⁺99.46%.

### Step 5. N-((1r,3r)-3-Aminocyclobutyl)-5-fluoro-N-isopropyl-2-((4-(6-((6-(2-methoxyethyl)-5,6,7,8-tetrahydro-1,6-naphthyridin-4-yl)oxy)-2-azaspiro[3.3]heptan-2-yl)pyrimidin-5-yl)oxy)benzamide

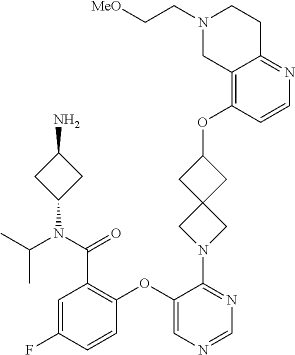 [SMILES: COCCN1CCc2nccc(OC3CC4(C3)CN(c3ncncc3Oc3ccc(F)cc3C(=O)N(C(C)C)[C@H]3C[C@H](N)C3)C4)c2C1]

This compound was synthesized following the procedure described for the synthesis of Example 20, step 3, starting with 1 equivalent of tert-butyl ((1r,3r)-3-(5-fluoro-N-isopropyl-2-((4-(6-((6-(2-methoxyethyl)-5,6,7,8-tetrahydro-1,6-naphthyridin-4-yl)oxy)-2-azaspiro[3.3]heptan-2-yl)pyrimidin-5-yl)oxy)benzamido)cyclobutyl)carbamate. After completion, the reaction was concentrated under reduced pressure to obtain crude N-((1r,3r)-3-aminocyclobutyl)-5-fluoro-N-isopropyl-2-((4-(6-((6-(2-methoxyethyl)-5,6,7,8-tetrahydro-1,6-naphthyridin-4-yl)oxy)-2-azaspiro[3.3]heptan-2-yl)pyrimidin-5-yl)oxy)benzamide xHCl. The crude was purified by reverse phase chromatography using a SiliCycle SiliaSep C18 column (80 g) on Biotage isolera one (Mobile phase A: 10 mM NH₄HCO₃ in water, B: ACN) to obtain N-((1r,3r)-3-aminocyclobutyl)-5-fluoro-N-isopropyl-2-((4-(6-((6-(2-methoxyethyl)-5,6,7,8-tetrahydro-1,6-naphthyridin-4-yl)oxy)-2-azaspiro[3.3]heptan-2-yl)pyrimidin-5-yl)oxy)benzamide as a solid.

Yield: 90%; ¹H NMR (400 MHz, DMSO-d): 6 8.28-8.26 (m, 1H), 8.18 (d, J=5.8 Hz, 1H), 7.74 (br s, 1H), 7.29-7.21 (m, 2H), 7.14-7.03 (m, 1H), 6.66 (d, J=5.8 Hz, 1H), 4.74 (quin, J=6.5 Hz, 1H), 4.41-4.08 (m, 5H), 3.76-3.62 (m, 1H), 3.51 (t, J=5.8 Hz, 2H), 3.48 (s, 2H), 3.26 (s, 3H), 3.23-2.97 (m, 2H), 2.81-2.73 (m, 6H), 2.70-2.67 (m, 2H), 2.30-2.21 (m, 3H), 1.85-1.59 (m, 2H), 1.47-1.27 (m, 3H), 1.09-0.94 (m, 3H), two protons merged with solvent peaks; LCMS (Method A): Rt=1.62 min, m/z=646.2 [M+H]⁺; HPLC (Method A): Rt=4.41 min, 99.70%; Chiral SFC (Method Y): Rt=3.42 min, 98.75%.

### Example 63. N-((1r,3r)-3-(Dimethylamino)cyclobutyl)-5-fluoro-N-isopropyl-2-((4-(6-((6-(2-methoxyethyl)-5,6,7,8-tetrahydro-1,6-naphthyridin-4-yl)oxy)-2-azaspiro[3.3]heptan-2-yl)pyrimidin-5-yl)oxy)benzamide (Compound No. 63)

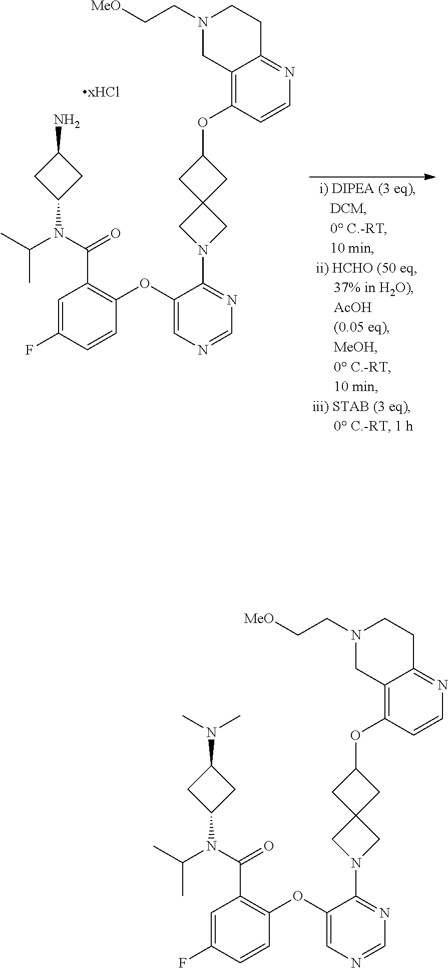 [SMILES: COCCN1CCc2nccc(OC3CC4(C3)CN(c3ncncc3Oc3ccc(F)cc3C(=O)N(C(C)C)[C@H]3C[C@H](N)C3)C4)c2C1 ; COCCN1CCc2nccc(OC3CC4(C3)CN(c3ncncc3Oc3ccc(F)cc3C(=O)N(C(C)C)[C@H]3C[C@H](N(C)C)C3)C4)c2C1]

This compound was synthesized following the procedure described for the synthesis of Example 20, step 4, starting with 1 equivalent of N-((1r,3r)-3-aminocyclobutyl)-5-fluoro-N-isopropyl-2-((4-(6-((6-(2-methoxyethyl)-5,6,7,8-tetrahydro-1,6-naphthyridin-4-yl)oxy)-2-azaspiro[3.3]heptan-2-yl)pyrimidin-5-yl)oxy)benzamide xHCl, except that 3 equivalents of DIPEA, 50 equivalents of formaldehyde (37% in H₂O), 0.05 equivalent of AcOH, and 3 equivalents of STAB were used. After completion, the reaction was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain N-((1r,3r)-3-(dimethylamino)cyclobutyl)-5-fluoro-N-isopropyl-2-((4-(6-((6-(2-methoxyethyl)-5,6,7,8-tetrahydro-1,6-naphthyridin-4-yl)oxy)-2-azaspiro[3.3]heptan-2-yl)pyrimidin-5-yl)oxy)benzamide as a solid.

Yield: 67.4%; ¹H NMR (400 MHz, DMSO-d₆): δ 8.30-8.24 (m, 1H), 8.18 (d, J=5.8 Hz, 1H), 7.76-7.63 (m, 1H), 7.31-7.22 (m, 2H), 7.16-7.06 (m, 1H), 6.67-6.63 (m, 1H), 4.75 (quin, J=6.6 Hz, 1H), 4.36-4.07 (m, 4H), 4.02-3.89 (m, 1H), 3.81-3.61 (m, 1H), 3.51 (t, J=5.8 Hz, 2H), 3.48 (br s, 2H), 3.26 (s, 3H), 2.99-2.88 (m, 1H), 2.83-2.72 (m, 7H), 2.68 (t, J=5.8 Hz, 2H), 2.29-2.23 (m, 2H), 2.15-1.77 (m, 9H), 1.48-1.29 (m, 3H), 1.07-0.88 (m, 3H); LCMS (Method C): Rt=1.52 min, m/z=674.3 [M+H]⁺; HPLC (Method A): Rt=4.77 min, 99.53%; Chiral SFC (Method J): Rt=4.61 min, 97.64%.

### Example 64. 5-Fluoro-N-isopropyl-N-(1-methylazetidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 64)

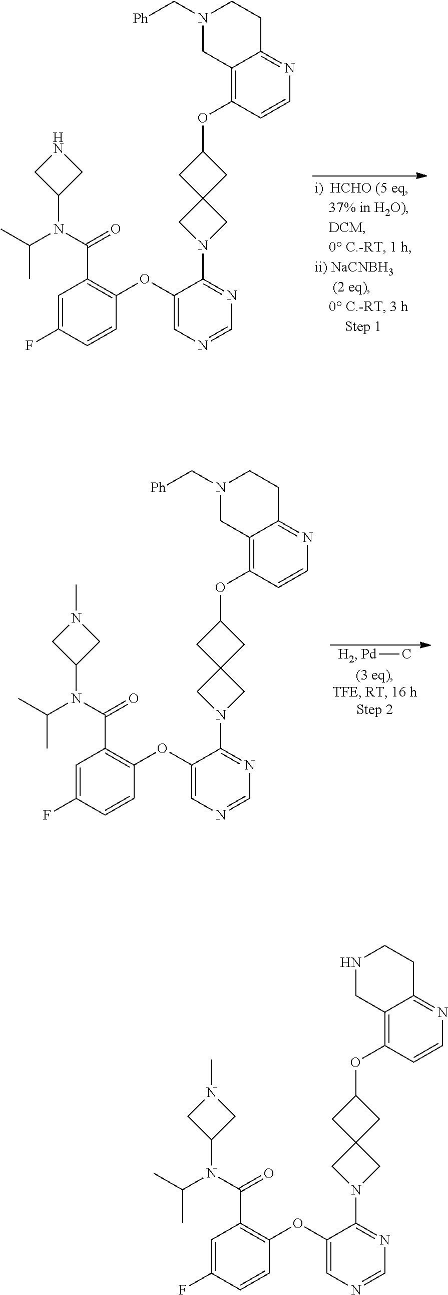

### Step 1. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(J-methylazetidin-3-yl)benzamide

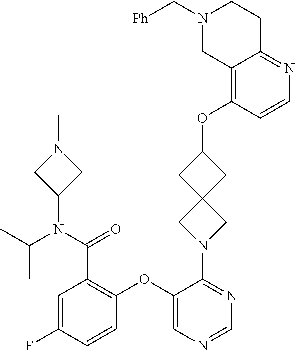 [SMILES: CC(C)N(C(=O)c1cc(F)ccc1Oc1cncnc1N1CC2(CC(Oc3ccnc4c3CN(Cc3ccccc3)CC4)C2)C1)C1CN(C)C1]

In a dried, 25 mL round bottom flask under a nitrogen atmosphere, N-(azetidin-3-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (220 mg, 0.331 mmol) was dissolved in MeOH (5 mL), and formaldehyde (134 mg, 1.657 mmol, 37% in H₂O) was added at 0° C. The reaction was stirred at RT for 1 h. To this reaction mixture, sodium cyanoborohydride (41.7 mg, 0.663 mmol) was added at 0° C. and the reaction was stirred at RT for 3 h. The reaction progress was monitored by TLC (10% MeOH in DCM). After completion, the reaction was concentrated under reduced pressure. The residue was diluted with saturated NaHCO₃ (10 mL) solution, and the mixture was extracted with DCM (2×10 mL). The combined organic layer was dried over sodium sulfate and filtered, and the filtrate was concentrated under reduced pressure to obtain crude 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-methylazetidin-3-yl)benzamide (140 mg, 57.1% yield) as a sticky liquid. This material was used without further purification. LCMS (Method F): Rt=1.87 min, m/z=678.4 [M+H]⁺, 91.64%.

### Step 2. 5-Fluoro-N-isopropyl-N-(1-methylazetidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

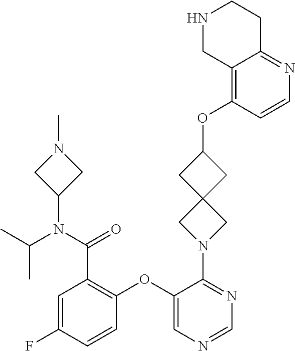 [SMILES: CC(C)N(C(=O)c1cc(F)ccc1Oc1cncnc1N1CC2(CC(Oc3ccnc4c3CNCC4)C2)C1)C1CN(C)C1]

This compound was synthesized following the procedure described for the synthesis of Example 20, step 5, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-methylazetidin-3-yl)benzamide, except that 3 equivalents of Pd-C was used. After workup, the crude compound was purified by reverse phase chromatography using a SiliCycle SiliaSep C18 column (40 g) on Biotage isolera one (Mobile phase A: 10 mM NH₄HCO₃ in water, B: ACN), then by Prep HPLC (Method C) The pure fractions were concentrated under reduced pressure and lyophilized to afford 5-fluoro-N-isopropyl-N-(1-methylazetidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (21.12% yield) as a solid. ¹H NMR (400 MHz, DMSO-d₆): δ 8.32-8.23 (m, 1H), 8.16 (d, J=5.6 Hz, 1H), 7.78-7.69 (m, 1H), 7.35-7.16 (m, 2H), 7.10-7.02 (m, 1H), 6.64 (d, J=5.6 Hz, 1H), 4.73 (quin, J=6.6 Hz, 1H), 4.35-3.96 (m, 6H), 3.70 (s, 2H), 3.60-3.44 (m, 1H), 3.26-3.07 (m, 3H), 2.95 (br t, J=5.7 Hz, 2H), 2.79 (br d, J=9.5 Hz, 2H), 2.71-2.65 (m, 2H), 2.32-2.14 (m, 5H), 1.50-0.96 (m, 6H), one proton merged with solvent peaks; LCMS (Method H): Rt=3.62 min, m/z=588.0 [M+H]⁺; HPLC (Method A): Rt=3.88 min, 96.59%.

### Example 65. (S)-5-Fluoro-N-isopropyl-N-(1-methylpyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 65)

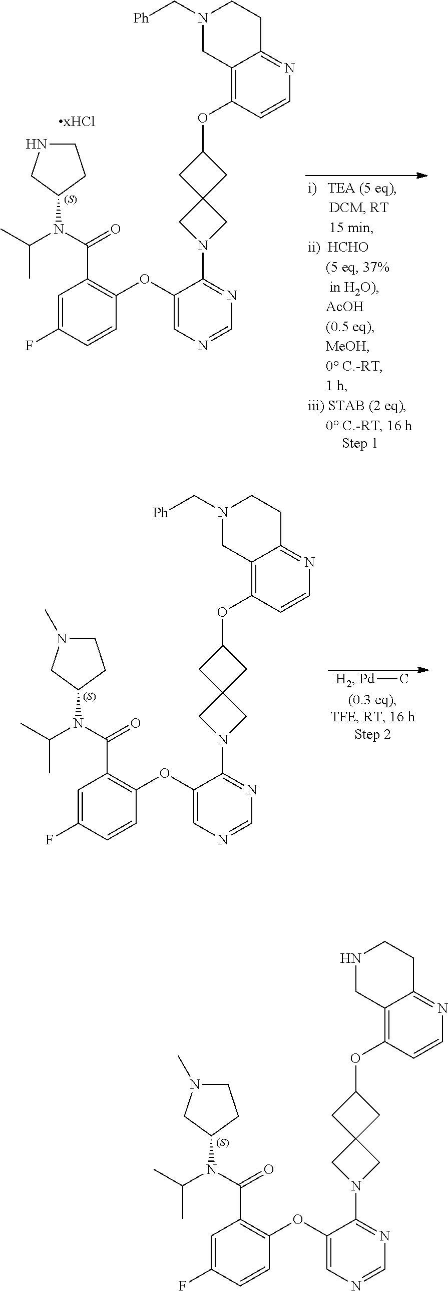

### Step 1. (S)-2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-methylpyrrolidin-3-yl)benzamide

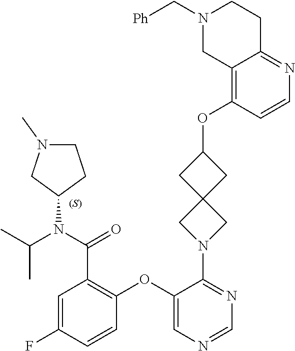

In a dried, round bottom flask under a nitrogen atmosphere, (S)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(pyrrolidin-3-yl)benzamide xHCl (110 mg, 0.154 mmol) was dissolved in DCM (1.2 mL). To this solution, TEA (0.107 mL, 0.770 mmol) was added, and the reaction was stirred at RT for 15 min. The reaction was concentrated under reduced pressure, and the residue was dissolved in MeOH (1.2 mL). To this reaction mixture, formaldehyde (0.077 mL, 0.770 mmol, 37% in H₂O) and acetic acid (4.40 μL, 0.077 mmol) were added at 0° C., and the reaction was stirred at RT for 1 h. Sodium cyanoborohydride (19.36 mg, 0.308 mmol) was added to this mixture at 0° C. and the reaction was stirred at RT for 16 h. The reaction was monitored by TLC (10% MeOH in DCM). After completion, the reaction was concentrated under reduced pressure. The residue was diluted with saturated NaHCO₃ solution (20 mL) and the mixture was extracted with DCM (3×20 mL). The combined organic layer was washed with brine (20 mL), dried over anhydrous sodium sulfate and filtered, and the filtrate was concentrated under reduced pressure to afford crude (S)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-methylpyrrolidin-3-yl)benzamide (80 mg, 54.6% yield) as a solid. This material was used without further purification. LCMS (Method A): Rt=1.91 min, m/z=692.5 [M+H]⁺, 72.74%.

### Step 2. (S)-5-Fluoro-N-isopropyl-N-(1-methylpyrrolidin-3-yl)-2-((4-(6-((5, 6,7, 8-tetrahydro-1,6-naphthyridin-4-yl)oxy)-2-azaspiro[3.3]heptan-2-yl)pyrimidin-5-yl)oxy)benzamide

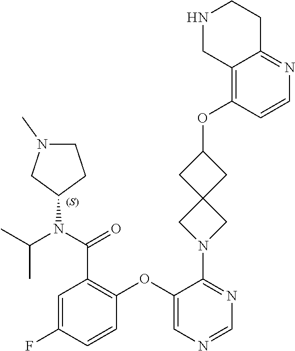

This compound was synthesized following the procedure described for the synthesis of Example 20, step 5, starting with 1 equivalent (S)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-methylpyrrolidin-3-yl)benzamide, except that 0.3 equivalent of Pd-C was used. The crude compound was purified by Prep HPLC (Method F). The pure fractions were concentrated under reduced pressure and lyophilized to afford (S)-5-fluoro-N-isopropyl-N-(1-methylpyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 43.6%; ¹H NMR (400 MHz, DMSO-d₆): δ 8.31-8.25 (m, 1H), 8.16 (d, J=5.6 Hz, 1H), 7.79-7.70 (m, 1H), 7.32-7.16 (m, 2H), 7.14-6.97 (m, 1H), 6.64 (br d, J=5.3 Hz, 1H), 4.79-4.66 (m, 1H), 4.37-4.03 (m, 5H), 3.96-3.77 (m, 1H), 3.70 (s, 2H), 2.95 (br t, J=5.5 Hz, 2H), 2.83-2.73 (m, 3H), 2.71-2.64 (m, 3H), 2.30-2.12 (m, 7H), 2.06-1.58 (m, 2H), 1.50-0.96 (m, 6H), one proton merged with solvent peaks; LCMS (Method H): Rt=2.95 min, m/z=602.3 [M+H]⁺; HPLC (Method A): Rt=3.89 min, 99.27%; Chiral SFC (Method M): Rt=2.58 min, 100%.

### Example 66. (R)-5-Fluoro-N-isopropyl-N-(1-methylpyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 66)

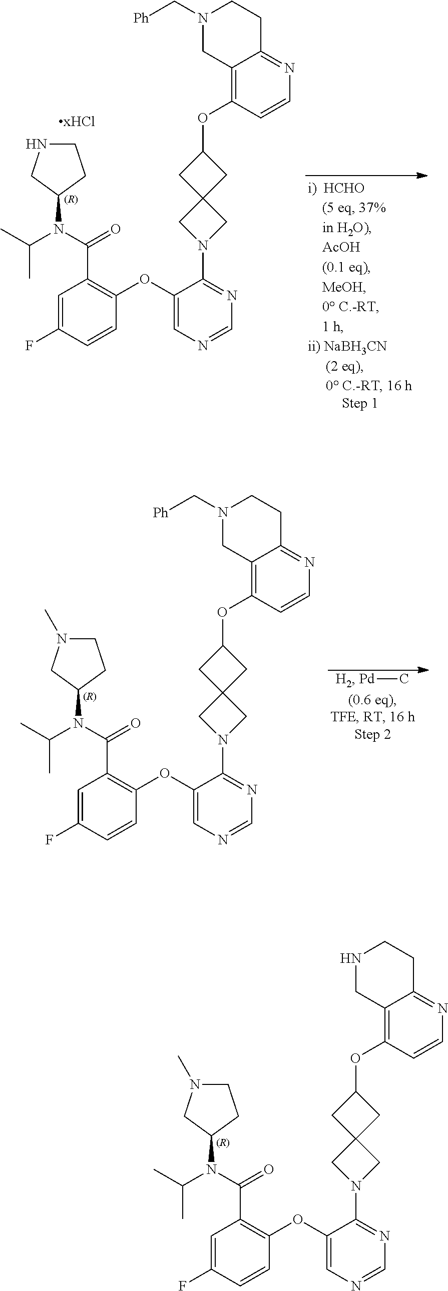

### Step 1. (R)-2-((4-(6-((6-Benzyl-5,6,7,8-tetrahydro-1,6-naphthyridin-4-yl]oxy)-2-azaspiro[3.3]heptan-2-y,)pyrimidin-5-yl)oxy)-5-fluoro-N-isopropyl-N-(1-methylpyrrolidin-3-yl)benzamide

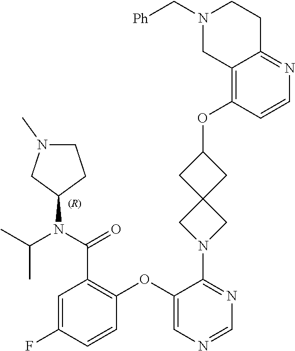

To a stirred solution of (R)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(pyrrolidin-3-yl)benzamide xHCl (0.2 g, 0.280 mmol) and formaldehyde (0.039 mL, 1.400 mmol, 37% in H₂O) in MeOH (4 mL), acetic acid (1.603 μL, 0.028 mmol) was added at 0° C. The reaction was stirred at RT for 1 h. The reaction was cooled to 0° C., and sodium cyanoborohydride (0.035 g, 0.560 mmol) was added. The reaction was stirred at RT for 16 h. The reaction progress was monitored by LCMS and TLC (20% MeOH in DCM). After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain (R)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-methylpyrrolidin-3-yl)benzamide (0.08 g, 31.1% yield) as a solid. LCMS (Method A): Rt=2.04 min, m/z=692.4 [M+H]⁺, 75.35%.

### Step 2. (R)-5-Fluoro-N-isopropyl-N-(1-methylpyrrolidin-3yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

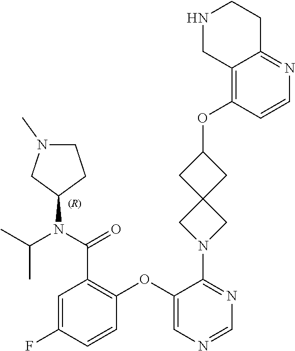

This compound was synthesized following the procedure described for the synthesis of Example 20, step 5, starting with 1 equivalent of (R)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-methylpyrrolidin-3-yl)benzamide, except that 0.6 equivalent of Pd-C was used. The crude was purified by reverse phase chromatography using a SiliCycle SiliaSep C18 column (40 g) on Biotage isolera one (Mobile phase A: 10 mM NH₄HCO₃ in water, B: ACN) to obtain crude (R)-5-fluoro-N-isopropyl-N-(1-methylpyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 33.0%; ¹H NMR (400 MHz, DMSO-d₆): δ 8.30-8.25 (m, 1H), 8.16 (d, J=5.7 Hz, 1H), 7.79-7.70 (m, 1H), 7.31-7.16 (m, 2H), 7.12-7.02 (m, 1H), 6.66-6.60 (m, 1H), 4.77-4.66 (m, 1H), 4.37-4.02 (m, 5H), 3.90-3.77 (m, 1H), 3.70 (s, 2H), 2.95 (br t, J=5.7 Hz, 2H), 2.84-2.71 (m, 4H), 2.70-2.58 (m, 4H), 2.30-2.12 (m, 5H), 2.07-1.72 (m, 2H), 1.52-0.98 (m, 6H), one proton merged with solvent peaks: LCMS (Method B): Rt=1.01 min, m/z=602.2 [M+H]⁺; HPLC (Method A): Rt=3.88 min, 97.74%; Chiral SFC (Method J): Rt=4.96 min, 94.74%.

### Example 67. 5-Fluoro-N-isopropyl-N-(1-(methylsulfonyl)azetidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 67)

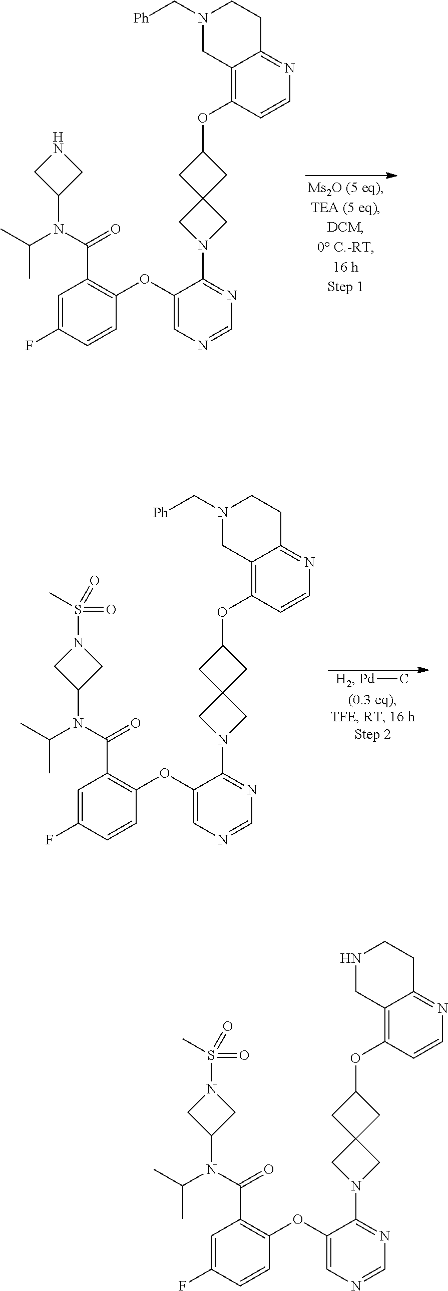

### Step 1. 2-(4-(6-(6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-(methylsulfonyl)azetidin-3-yl)benzamide

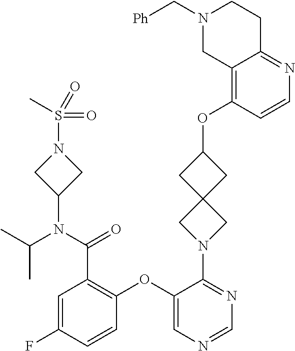 [SMILES: CC(C)N(C(=O)c1cc(F)ccc1Oc1cncnc1N1CC2(CC(Oc3ccnc4c3CN(Cc3ccccc3)CC4)C2)C1)C1CN(S(C)(=O)=O)C1]

To a stirred solution of N-(azetidin-3-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (200 mg, 0.301 mmol) in DCM (3 mL), TEA (0.126 mL, 0.904 mmol) and methanesulfonic anhydride (262 mg, 1.506 mmol) were added at 0° C. The reaction was stirred at RT for 16 h. After completion, the reaction was concentrated under reduced pressure. The crude compound was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure to afford 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-(methylsulfonyl)azetidin-3-yl)benzamide (80 mg, 35.2% yield) as a solid. LCMS (Method F): Rt=2.19 min, m/z=742.5 [M+H]⁺,98.39%.

### Step 2. 5-Fluoro-N-isopropyl-N-(1-(methylsulfonyl)azetidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

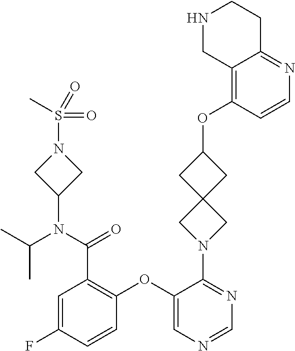 [SMILES: CC(C)N(C(=O)c1cc(F)ccc1Oc1cncnc1N1CC2(CC(Oc3ccnc4c3CNCC4)C2)C1)C1CN(S(C)(=O)=O)C1]

This compound was synthesized following the procedure described for the synthesis of Example 20, step 5, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-(methylsulfonyl)azetidin-3-yl)benzamide, except that 0.3 equivalent of Pd-C was used. After filtration and concentration, the crude was purified by Prep HPLC (Method C), then by achiral Prep SFC (Method A). The pure fractions were concentrated under reduced pressure and lyophilized to obtain 5-fluoro-N-isopropyl-N-(1-(methylsulfonyl)azetidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (39.6% yield) as a solid. ¹H NMR (400 MHz, DMSO-d₆): δ 8.33-8.28 (m, 1H), 8.16 (d, J=5.6 Hz, 1H), 7.93-7.80 (m, 1H), 7.40 (dd, J=3.0, 8.1 Hz, 1H), 7.32-7.22 (m, 1H), 7.04 (dd, J=4.3, 9.1 Hz, 1H), 6.64 (d, J=5.6 Hz, 1H), 4.81-4.67 (m, 2H), 4.60-4.49 (m, 1H), 4.47-4.37 (m, 1H), 4.34-4.00 (m, 4H), 3.98-3.87 (m, 2H), 3.78-3.72 (m, 1H), 3.70 (s, 2H), 3.07-2.99 (m, 3H), 2.96 (br t, J=5.7 Hz, 2H), 2.82-2.74 (m, 2H), 2.71-2.65 (m, 2H), 2.27-2.20 (m, 2H), 1.56-0.95 (m, 6H), one proton merged with solvent peaks; LCMS (Method F): Rt=1.57 min, m/z=652.2 [M+H]⁺; HPLC (Method A): Rt=4.38 min, 99.02%.

### Example 68. (S)—N-(1-Acetylpyrrolidin-3-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 68)

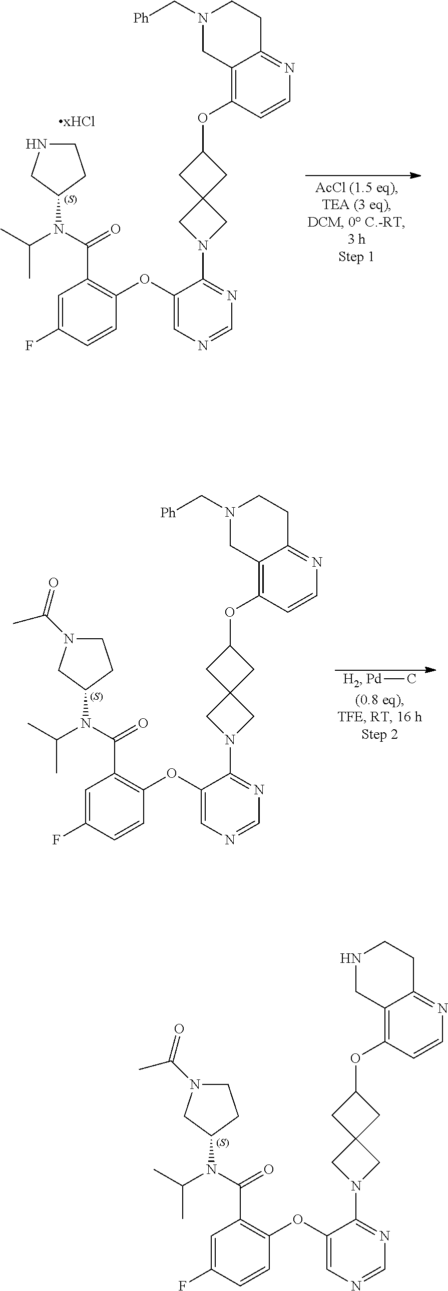

### Step 1. (S)—N-(1-Acetylpyrrolidin-3-yl)-2-((4-(6-((6-benzyl-5,6,7,8-tetrahydro-1,6-naphthyridin-4-yl)oxy)-2-azaspiro[3.3]heptan-2-ylpyrimidin-1-yl)oxy)-5-fluoro-N-isopropylbenzamide

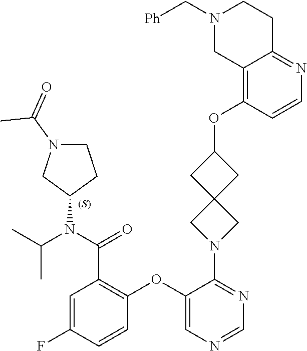

In a dried, 25 mL round bottom flask under a nitrogen atmosphere, (S)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(pyrrolidin-3-yl)benzamide xHCl (250 mg, 0.350 mmol) in DCM (2 mL), triethylamine (0.146 mL, 1.050 mmol) was added at RT. The reaction was stirred at RT for 5 min. To this mixture, acetyl chloride (0.037 mL, 0.525 mmol)) was added at 0° C., and the reaction was stirred at RT for 3 h. The reaction progress was monitored by LCMS and TLC (10% MeOH in DCM). After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure to afford (S)—N-(1-acetylpyrrolidin-3-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (130 mg, 44.5% yield). LCMS (Method B): Rt=1.18 min, m/z=720.2 [M+H]⁺, 86.28%.

### Step 2. (—S)—N-(1Acetylpyrrolidin-3-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

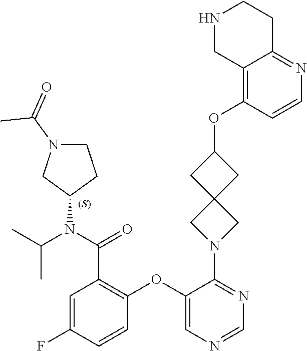

This compound was synthesized following the procedure described for the synthesis of Example 20, step 5, starting with 1 equivalent (S)—N-(1-acetylpyrrolidin-3-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide, except that 0.8 equivalent of Pd-C was used. After filtration and concentration, the crude was purified by reverse phase chromatography using a SiliCycle SiliaSep C18 column (40 g) on Biotage isolera one (Mobile phase A: 10 mM NH₄HCO₃ in water, B: ACN) to obtain (S)—N-(1-acetylpyrrolidin-3-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 14.98%; ¹H NMR (400 MHz, DMSO-d₆): δ 8.32-8.27 (m, 1H), 8.16 (br d, J=5.5 Hz, 1H), 7.82-7.77 (m, 1H), 7.37-7.21 (m, 2H), 7.07-6.98 (m, 1H), 6.70-6.60 (m, 1H), 4.77-4.67 (m, 1H), 4.32-3.93 (m, 5H), 3.79-3.71 (m, 2H), 3.69 (br s, 2H), 3.66-3.40 (m, 2H), 2.94 (br t, J=5.6 Hz, 2H), 2.84-2.73 (m, 3H), 2.70-2.64 (m, 3H), 2.29-2.20 (m, 2H), 1.96-1.82 (m, 4H), 1.47-0.99 (m, 6H), one proton merged with solvent peaks; LCMS (Method A): Rt=1.43 min, m/z=630.3 [M+H]⁺; HPLC (Method A): Rt=4.11 min, 96.80%; Chiral SFC (Method N): Rt=1.88 min, 97.51%.

### Example 69. (S)-5-Fluoro-N-isopropyl-N-(1-(methylsulfonyl)pyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 69)

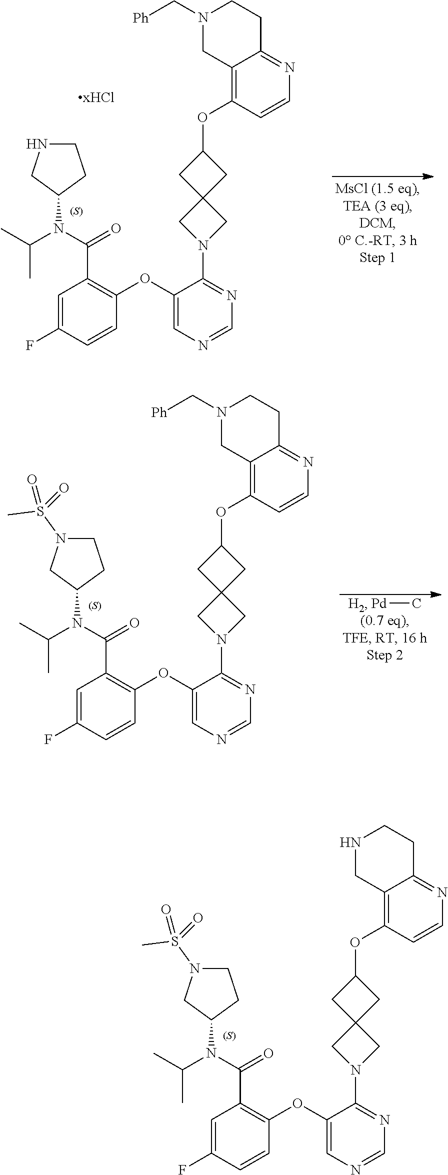

### Step 1. (S)-2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-(methylsulfonyl)pyrrolidin-3-yl)benzamide

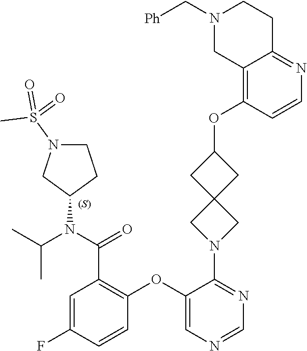

This compound was synthesized following the procedure described for the synthesis of Example 68, step 1, starting with 1 equivalent (S)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(pyrrolidin-3-yl)benzamide xHCl, except that 1.5 equivalents of methanesulfonyl chloride was used. The crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure to afford (S)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-(methylsulfonyl)pyrrolidin-3-yl)benzamide as a solid.

Yield: 34.2%; LCMS (Method B): Rt=1.23 min, m/z=756.0 [M+H]⁺, 82.31%.

### Step 2. (S)—5-Fluoro-N-isopropyl-N-(1-(methylsulfonyl)pyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

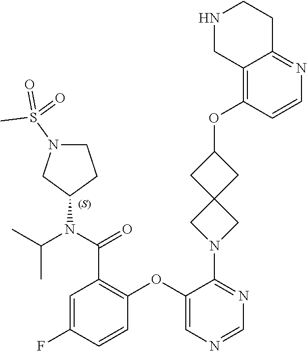

This compound was synthesized following the procedure described for the synthesis of Example 20, step 5, starting with 1 equivalent of (S)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-(methylsulfonyl)pyrrolidin-3-yl)benzamide, except that 0.7 equivalent of Pd-C was used. After filtration and concentration, the crude compound was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford (S)-5-fluoro-N-isopropyl-N-(1-(methylsulfonyl)pyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 27.0%; ¹H NMR (400 MHz, DMSO-d₆): δ 8.32-8.27 (m, 1H), 8.19-8.12 (m, 1H), 7.85-7.74 (m, 1H), 7.42-7.32 (m, 1H), 7.32-7.21 (m, 11H), 7.11-6.97 (m, 1H), 6.64 (d, J=5.6 Hz, 1H), 4.72 (spt, J=6.1 Hz, 1H), 4.31-4.08 (m, 5H), 3.80-3.71 (m, 1H), 3.69 (s, 2H), 3.61-3.47 (m, 2H), 3.45-3.37 (m, 1H), 2.97-2.86 (m, 5H), 2.83-2.72 (m, 2H), 2.70-2.65 (m, 2H), 2.62-2.55 (m, 1H), 2.43-2.35 (m, 1H), 2.28-2.21 (m, 2H), 2.11-1.95 (m, 1H), 1.49-1.01 (m, 6H), one proton merged with solvent peaks; LCMS (Method H): Rt=3.06 min, m/z=666.3 [M+H]⁺; HPLC (Method A): Rt=4.47 min, 97.15%; Chiral SFC (Method O): Rt=1.43 min, 100%.

### Example 70. (R)—N-(1-Acetylpyrrolidin-3-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 70)

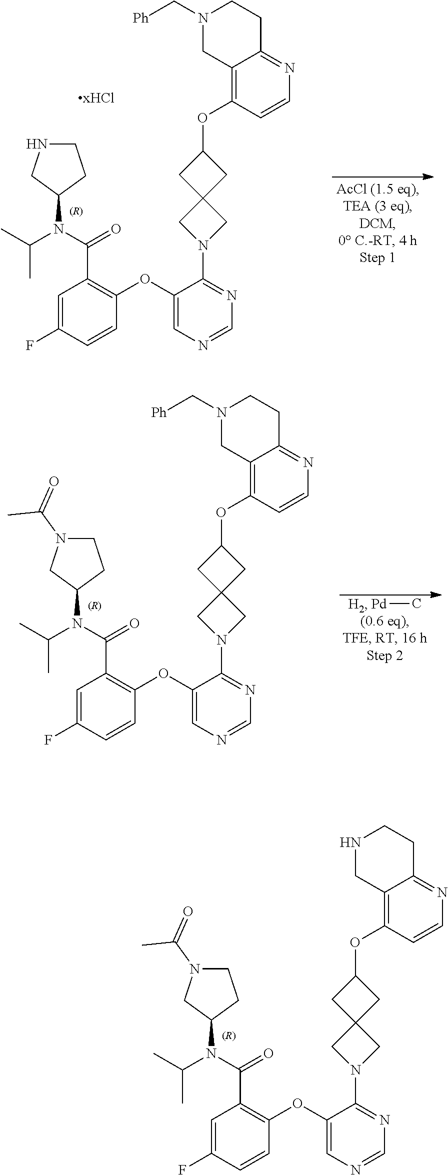

### Step 1. (R)—N-(Acetylpyrrolidin-3 yl)-2-((4-(6-((6-benz-1-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide

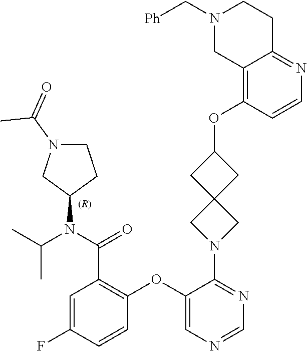

This compound was synthesized following the procedure described for the synthesis of Example 68, step 1, starting with 1 equivalent of (R)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(pyrrolidin-3-yl)benzamide xHCl and 1.5 equivalents of acetyl chloride. The crude was purified by reverse phase chromatography using a SiliCycle SiliaSep C18 column (40 g) on Biotage isolera one (Mobile phase A: 10 mM NH₄HCO₃ in water, B: ACN) to obtain (R)—N-(1-acetylpyrrolidin-3-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide as a solid.

Yield: 69.7%; LCMS (Method A): Rt=1.82 min, m/z=720.3 [M+H]⁺, 92.54%.

### Step 2. (R)—N-(1-Acetylpyrrolidin-3-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

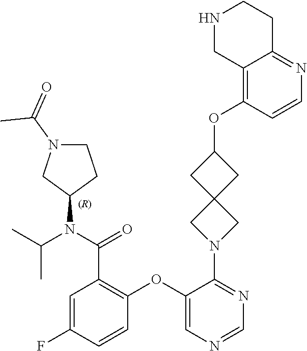

This compound was synthesized following the procedure described for the synthesis of Example 20, step 5, starting with 1 equivalent of (R)—N-(1-acetylpyrrolidin-3-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide, except that 0.6 equivalent of Pd-C was used. After filtration and concentration, the crude was purified by reverse phase chromatography using a SiliCycle SiliaSep C18 column (40 g) on Biotage isolera one (Mobile phase A: 10 mM NH₄HCO₃ in water, B: ACN) to obtain (R)—N-(1-acetylpyrrolidin-3-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 62.9%; ¹H NMR (400 MHz, DMSO-d₆): δ 8.33-8.27 (m, 1H), 8.21-8.13 (m, 1H), 7.87-7.72 (m, 1H), 7.38-7.21 (m, 2H), 7.10-6.97 (m, 1H), 6.72-6.61 (m, 1H), 4.78-4.64 (m, 1H), 4.34-3.92 (m, 5H), 3.80-3.72 (m, 1H), 3.69 (br s, 2H), 3.65-3.39 (m, 3H), 3.27-3.19 (m, 1H), 2.94 (br t, J=5.7 Hz, 2H), 2.84-2.73 (m, 2H), 2.67 (br t, J=5.4 Hz, 2H), 2.29-2.18 (m, 2H), 2.02-1.81 (m, 4H), 1.48-0.98 (m, 6H), two protons merged with solvent peaks; LCMS (Method A): Rt=1.41 min, m/z=630.3 [M+H]⁺; HPLC (Method A): Rt=4.05 min, 98.73%; Chiral SFC (Method N). Rt=1.66 min, 95.76%.

### Example 71. (R)-5-Fluoro-N-isopropyl-N-(1-(methylsulfonyl)pyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 71)

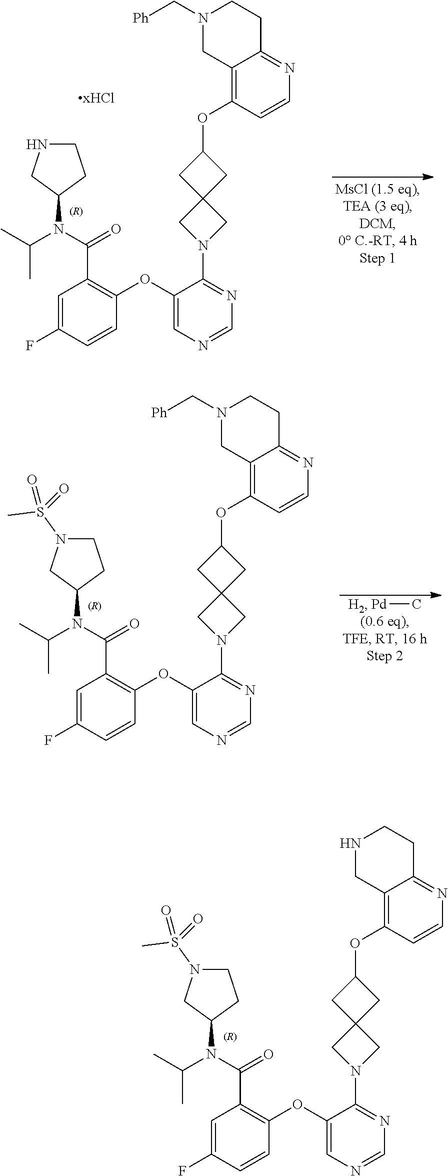

### Step 1. (R)-2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-(methylsulfonyl)pyrrolidin-3-yl)benzamide

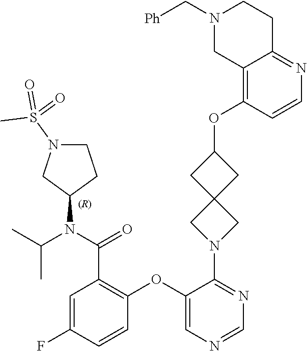

This compound was synthesized following the procedure described for the synthesis of Example 68, step 1, starting with 1 equivalent of (R)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(pyrrolidin-3-yl)benzamide xHCl and 1.5 equivalents methanesulfonyl chloride. The crude was purified by reverse phase chromatography using a SiliCycle SiliaSep C18 column (40 g) on Biotage isolera one (Mobile phase A: 10 mM NH₄HCO₃ in water, B: ACN) to obtain (R)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-(methylsulfonyl)pyrrolidin-3-yl)benzamide as a solid.

Yield: 73.2%; ¹H NMR (400 MHz, DMSO-d₆): δ 8.32-8.26 (m, 1H), 8.22-8.14 (m, 1H), 7.82-7.75 (m, 1H), 7.41-7.31 (m, 5H), 7.30-7.21 (m, 2H), 7.11-6.95 (m, 1H), 6.66 (d, J=5.6 Hz, 1H), 4.79-4.64 (m, 1H), 4.31-4.08 (m, 5H), 3.78-3.70 (m, 1H), 3.68 (s, 2H), 3.62-3.42 (m, 3H), 2.96-2.85 (m, 3H), 2.84-2.68 (m, 6H), 2.27-2.16 (m, 2H), 2.10-1.93 (m, 1H), 1.49-0.98 (m, 6H), four protons merged with solvent peaks; LCMS (Method A): Rt=1.96 min, m/z=756.2 [M+H]⁺, 95.79%.

### Step 2. (R)-5-fluoro-N-isopropyl-N-(1-(methylsulfonyl)pyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

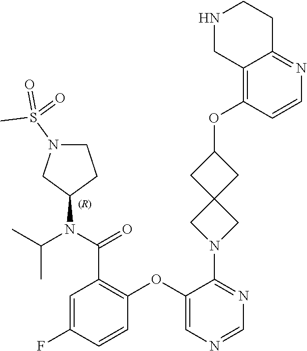

This compound was synthesized following the procedure described for the synthesis of Example 20, step 5, starting with 1 equivalent of (R)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-(methylsulfonyl)pyrrolidin-3-yl)benzamide, except that 0.6 equivalent of Pd-C was used. The crude was purified by reverse phase chromatography using a SiliCycle SiliaSep C18 column (40 g) on Biotage isolera one (Mobile phase A: 10 mM NH₄HCO₃ in water, B: ACN) to obtain (R)-5-fluoro-N-isopropyl-N-(1-(methylsulfonyl)pyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 36.1%; ¹H NMR (400 MHz, DMSO-d₆): δ 8.32-8.25 (m, 1H), 8.22-8.13 (m, 1H), 7.84-7.74 (m, 1H), 7.42-7.30 (m, 11H), 7.29-7.19 (m, 1H), 7.12-6.98 (m, 1H), 6.69-6.62 (m, 1H), 4.80-4.65 (m, 1H), 4.34-4.04 (m, 5H), 3.83-3.64 (m, 2H), 3.60-3.50 (m, 3H), 3.49-3.36 (m, 1H), 3.30-3.25 (m, 2H), 2.95-2.73 (m, 9H), 2.31-2.21 (m, 2H), 2.10-1.93 (m, 1H), 1.49-0.99 (m, 6H), one proton merged with solvent peaks; LCMS (Method A): Rt=1.52 min, m/z=666.2 [M+H]⁺; HPLC (Method A): Rt=4.46 min, 99.41%; Chiral SFC (Method H): Rt=5.45 min, 88.25%.

### Example 72. N-(1-(2,2-Difluoroethyl)azetidin-3-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 72)

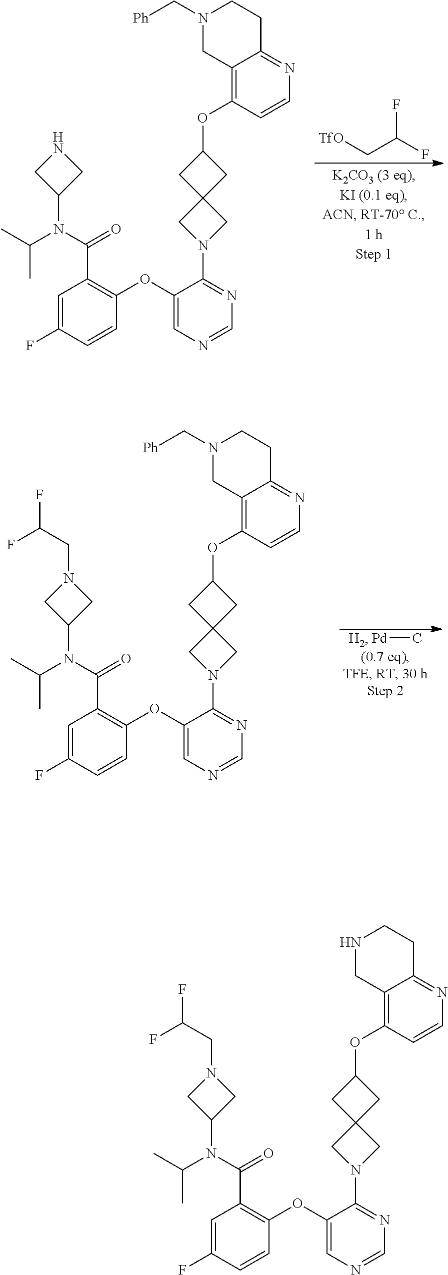

### Step 1. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-N-(1-(2,2-difluoroethyl)azetidin-3-yl)-5-fluoro-N-isopropylbenzamide

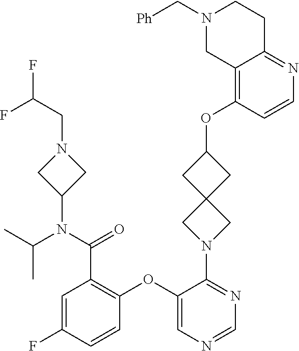 [SMILES: CC(C)N(C(=O)c1cc(F)ccc1Oc1cncnc1N1CC2(CC(Oc3ccnc4c3CN(Cc3ccccc3)CC4)C2)C1)C1CN(CC(F)F)C1]

In a 20 mL pressure vial under a nitrogen atmosphere, N-(azetidin-3-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (200 mg, 0.301 mmol) and 2,2-difluoroethyl trifluoromethanesulfonate (64.5 mg, 0.301 mmol) was dissolved in ACN (10 mL). To this solution, KI (5 mg, 0.030 mmol) and K₂CO₃ (125 mg, 0.904 mmol) were added at RT, and the reaction was stirred at 70° C. for 1 h. The reaction progress was monitored by TLC (10% MeOH in DCM). The reaction was concentrated under reduced pressure, the residue was diluted with water (5 mL), and the mixture was extracted with DCM (2×10 mL). The combined organic layer was dried over sodium sulfate and filtered, and the filtrate was concentrated under reduced pressure to obtain crude 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-(1-(2,2-difluoroethyl)azetidin-3-yl)-5-fluoro-N-isopropylbenzamide (210 mg, 90% yield) as a solid. This material was used without further purification. LCMS (Method F): Rt=2.01 min, m/z=728.2 [M+H]⁺, 94.43%.

### Step 2. N-(J-(2,2-Difluoroethyl)azetidin-3-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

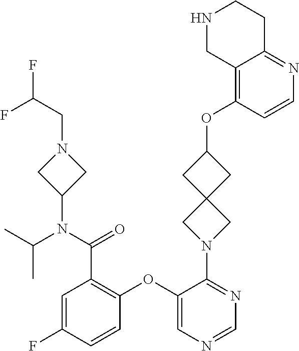 [SMILES: CC(C)N(C(=O)c1cc(F)ccc1Oc1cncnc1N1CC2(CC(Oc3ccnc4c3CNCC4)C2)C1)C1CN(CC(F)F)C1]

This compound was synthesized following the procedure described for the synthesis of Example 20, step 5, starting with 1 equivalent of N-(azetidin-3-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide, except that 0.7 equivalent of Pd-C was used. After filtration and concentration, the crude was purified by reverse phase chromatography using a SiliCycle SiliaSep C18 column (40 g) on Biotage isolera one (Mobile phase A: 10 mM NH₄HCO₃ in water, B: ACN) to obtain N-(1-(2,2-difluoroethyl)azetidin-3-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (26.4% yield) as a solid. ¹H NMR (400 MHz, DMSO-d₆): δ 8.33-8.23 (m, 1H), 8.15 (d, J=5.6 Hz, 1H), 7.79-7.70 (m, 1H), 7.37-7.16 (m, 2H), 7.12-7.01 (m, 1H), 6.63 (br d, J=5.0 Hz, 1H), 6.12-5.66 (m, 1H), 4.72 (quin, J=6.6 Hz, 1H), 4.33-4.07 (m, 6H), 4.00-3.90 (m, 1H), 3.87-3.77 (m, 1H), 3.69 (s, 2H), 3.63-3.53 (m, 1H), 3.44-3.37 (m, 1H), 3.22-3.10 (m, 1H), 3.01-2.88 (m, 3H), 2.87-2.73 (m, 3H), 2.70-2.65 (m, 2H), 2.29-2.19 (m, 2H), 1.51-0.90 (m, 6H); LCMS (Method A): Rt=1.56 min, m/z=638.4 [M+H]⁺; HPLC (Method A): Rt=4.51 min, 96.99%.

### Example 73. N-(3-Acetamidobicyclo[1.1.1]pentan-1-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 73)

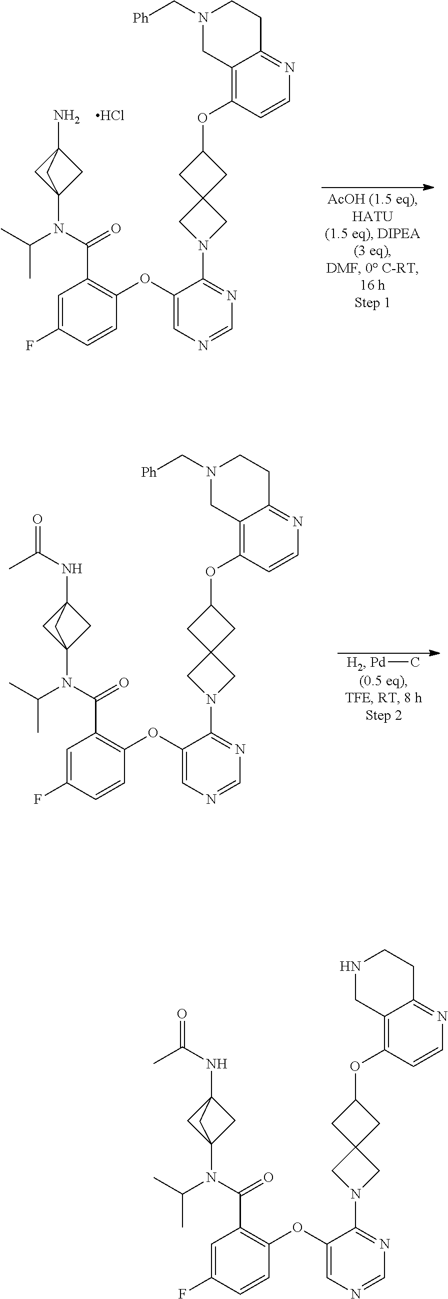

### Step 1. N-(3-Acetamidobicyclo[1.1.1]pentan-1-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide

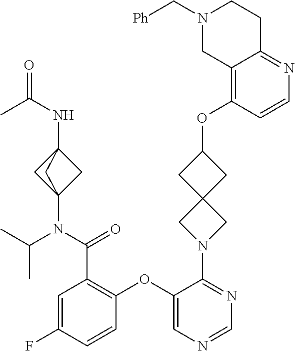 [SMILES: CC(=O)NC12CC(N(C(=O)c3cc(F)ccc3Oc3cncnc3N3CC4(CC(Oc5ccnc6c5CN(Cc5ccccc5)CC6)C4)C3)C(C)C)(C1)C2]

This compound was synthesized following the procedure described for the synthesis of Example 1, step 2, starting with 1 equivalent of N-(3-aminobicyclo[1.1.1]pentan-1-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl and 1.5 equivalents of acetic acid. After completion, the reaction was diluted with ice-cold water and the mixture was extracted with EtOAc. The combined organic layer was washed with brine solution, dried over anhydrous sodium sulfate and filtered, and the filtrate was concentrated under reduced pressure The crude compound was purified by reverse phase chromatography using a SiliCycle SiliaSep C18 column (80 g) on Biotage isolera one (Mobile phase A: 10 mM NH₄HCO₃ in water, B: ACN) to obtain N-(3-acetamidobicyclo[1.1.1]pentan-1-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide as a solid.

Yield: 45.3%; LCMS (Method A): Rt=1.88 min, m/z=732.3 [M+H]⁺, 91.39%.

### Step 2. N-(3-Acetamidobicyclo[1.1.1]pentan-1-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

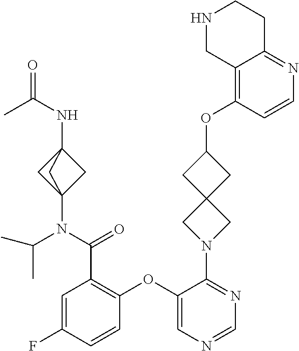 [SMILES: CC(=O)NC12CC(N(C(=O)c3cc(F)ccc3Oc3cncnc3N3CC4(CC(Oc5ccnc6c5CNCC6)C4)C3)C(C)C)(C1)C2]

This compound was synthesized following the procedure described for the synthesis of Example 20, step 5, starting with 1 equivalent of N-(3-acetamidobicyclo[1.1.1]pentan-1-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide, except that 0.5 equivalent of Pd-C was used. After filtration and concentration, the crude was purified by Prep HPLC (Method J) to obtain N-(3-acetamidobicyclo[1.1.1]pentan-1-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 49.8%; ¹H NMR (400 MHz, DMSO-d₆): δ 8.48-8.38 (m, 1H), 8.28 (br s, 1H), 8.16 (d, J=5.6 Hz, 1H), 7.81-7.62 (m, 11H), 7.34-7.21 (m, 2H), 7.12-6.98 (m, 1H), 6.66 (d, J=5.1 Hz, 1H), 4.84-4.66 (m, 1H), 4.34-4.08 (m, 5H), 4.07-3.76 (m, 1H), 3.70 (s, 2H), 2.95 (t, J=5.8 Hz, 2H), 2.86-2.73 (m, 2H), 2.71-2.65 (m, 2H), 2.30-2.19 (m, 2H), 2.05-1.91 (m, 4H), 1.81-1.68 (m, 3H), 1.39-1.07 (m, 6H), two protons merged with solvent peaks; LCMS (Method A): Rt=1.42 min, m/z=642.2 [M+H]⁺; HPLC (Method A): Rt=4.15 min, 99.89%.

### Example 74. N-((1r,3r)-3-(Dimethylamino)cyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 74)

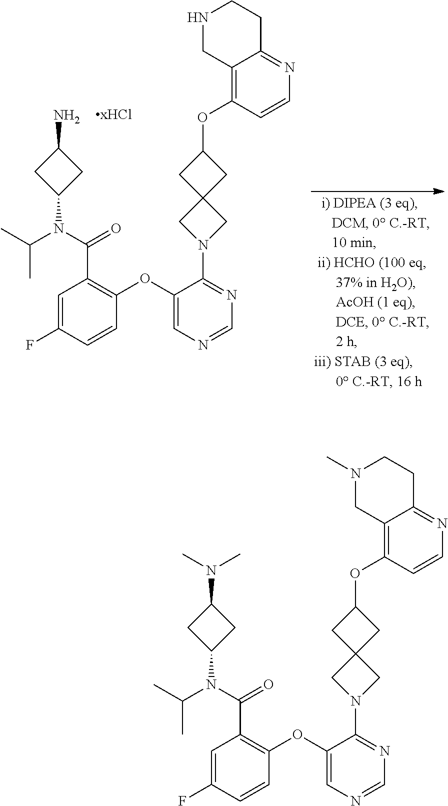 [SMILES: CC(C)N(C(=O)c1cc(F)ccc1Oc1cncnc1N1CC2(CC(Oc3ccnc4c3CNCC4)C2)C1)[C@H]1C[C@H](N)C1 ; CC(C)N(C(=O)c1cc(F)ccc1Oc1cncnc1N1CC2(CC(Oc3ccnc4c3CN(C)CC4)C2)C1)[C@H]1C[C@H](N(C)C)C1]

This compound was synthesized following the procedure described for the synthesis of Example 20, step 4, starting with 1 equivalent of N-((1r,3r)-3-aminocyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl, except that 3 equivalents of DIPEA, 100 equivalents of formaldehyde (37% in H₂O), 1 equivalent of AcOH, 3 equivalents of STAB were used, and DCE was used as the solvent. After filtration and concentration, the crude was purified by Prep HPLC (Method B) to afford N-((1r,3r)-3-(dimethylamino)cyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 22.57%; ¹H NMR (400 MHz, DMSO-d₆): δ 8.29-8.26 (m, 1H), 8.18 (d, J=5.5 Hz, 1H), 7.76-7.64 (m, 1H), 7.31-7.22 (m, 2H), 7.16-7.05 (m, 1H), 6.68-6.63 (m, 1H), 4.75 (quin, J=6.5 Hz, 1H), 4.34-4.06 (m, 5H), 4.01-3.90 (m, 1H), 3.80-3.62 (m, 1H), 3.37 (s, 2H), 2.99-2.89 (m, 1H), 2.88-2.74 (m, 5H), 2.66-2.60 (m, 2H), 2.36 (s, 3H), 2.30-2.19 (m, 2H), 2.18-2.06 (m, 2H), 2.04 (s, 3H), 1.95 (s, 3H), 1.49-1.28 (m, 3H), 1.08-0.89 (m, 3H); LCMS (Method A): Rt=1.65 min, m/z=630.5 [M+H]⁺; HPLC (Method A): Rt=4.57 min, 99.82%; Chiral SFC (Method J): Rt=4.88 min, 100.0%.

### Example 75. N-((1s,3s)-3-(Dimethylamino)cyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 75)

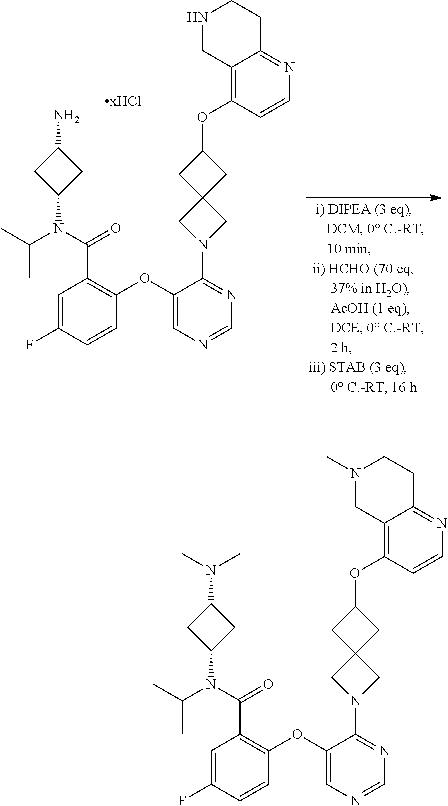

This compound was synthesized following the procedure described for the synthesis of Example 20, step 4, starting with 1 equivalent of N-((1s,3s)-3-aminocyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl, except that 3 equivalents of DIPEA, 70 equivalents of formaldehyde (37% in H₂O), 1 equivalent of AcOH, and 3 equivalents of STAB were used, and DCE was used as the solvent. After filtration and concentration, the crude was purified by reverse phase chromatography using a SiliCycle SiliaSep C18 column (80 g) on Biotage isolera one (Mobile phase A: 10 mM NH₄HCO₃ in water, B: ACN) to obtain N-((1s,3s)-3-(dimethylamino)cyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 42.6%; ¹H NMR (400 MHz, DMSO-d₆): δ 8.30-8.25 (m, 1H), 8.18 (d, J=5.6 Hz, 1H), 7.79-7.74 (m, 1H), 7.31-7.21 (m, 1H), 7.17-7.02 (m, 2H), 6.66 (d, J=5.6 Hz, 1H), 4.78-4.71 (m, 1H), 4.34-4.08 (m, 4H), 3.79-3.53 (m, 2H), 3.36 (s, 2H), 2.85-2.74 (m, 4H), 2.65-2.60 (m, 2H), 2.36 (s, 3H), 2.30-2.21 (m, 2H), 2.20-2.07 (m, 2H), 2.03 (s, 3H), 1.96 (s, 3H), 1.89-1.77 (m, 1H), 1.47-1.25 (m, 3H), 1.12-0.96 (m, 3H); LCMS (Method J): Rt=1.80 min, m/z=630.3 [M+H]⁺; HPLC (Method A): Rt=4.77 min, 97.01%; Chiral SFC (Method J): Rt=4.42 min, 98.93%.

### Example 76. N-((1s,4s)-4-(Dimethylamino)cyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 76)

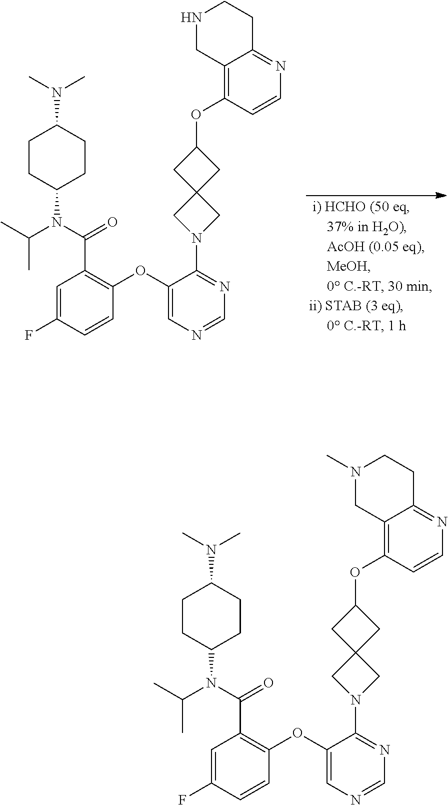

To a stirred solution of N-((1s,4s)-4-(dimethylamino)cyclohexyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (0.050 g, 0.078 mmol) in MeOH (2 mL), formaldehyde (0.107 mL, 3.88 mmol, 37% in H₂O) and acetic acid (0.222 μL, 3.88 μmol) were added at 0° C. The reaction was stirred at RT for 30 min. To this reaction mixture, STAB (0.049 g, 0.233 mmol) was added at 0° C., and the reaction was stirred at RT for 1 h. The reaction progress was monitored by LCMS. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain N-((1s,4s)-4-(dimethylamino)cyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (17 mg, 33.0% yield). ¹H NMR (400 MHz, DMSO-d₆): δ 8.26 (s, 1H), 8.20-8.16 (m, 1H), 7.72-7.68 (m, 1H), 7.29-7.19 (m, 2H), 7.13-7.08 (m, 1H), 6.66 (d, J=5.8 Hz, 1H), 4.79-4.71 (m, 1H), 4.33-4.10 (m, 4H), 3.71-3.39 (m, 1H), 3.36 (s, 2H), 3.29-3.20 (m, 1H), 2.84-2.74 (m, 4H), 2.65-2.60 (m, 2H), 2.36 (s, 3H), 2.30-2.20 (m, 2H), 2.14-2.07 (m, 6H), 2.02-1.84 (m, 2H), 1.83-1.71 (m, 3H), 1.48-0.91 (m, 10H); LCMS (Method C): Rt=1.41 min, m/z=658.3 [M+H]⁺; HPLC (Method A): Rt=4.99 min, 99.65%; Chiral SFC (Method C): Rt=3.81 min, 98.48%.

### Example 77. N-((1r,3r)-3-(Dimethylamino)cyclobutyl)-5-fluoro-2-((4-(6-((6-isobutyl-5,6,7,8-tetrahydro-1,6-naphthyridin-4-yl)oxy)-2-azaspiro[3.3]heptan-2-yl)pyrimidin-5-yl)oxy)-N-isopropylbenzamide (Compound No. 77)

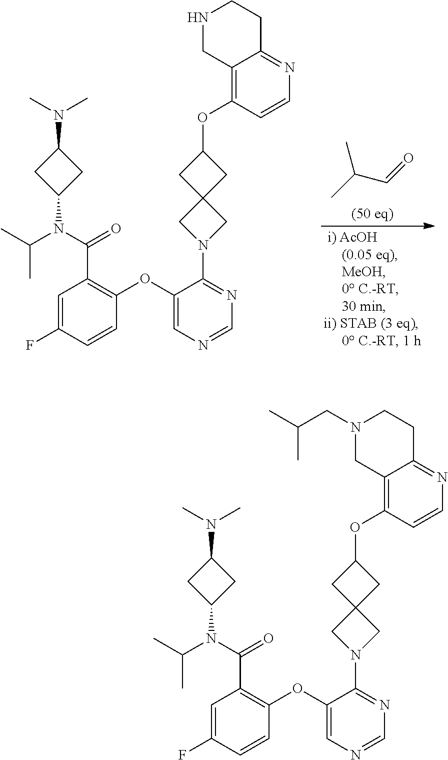

This compound was synthesized following the procedure described for the synthesis of Example 76, starting with 1 equivalent of N-((1r,3r)-3-(dimethylamino)cyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide and 50 equivalents of isobutyraldehyde. After completion, the crude was purified by reverse phase chromatography using a SiliCycle SiliaSep C18 column (80 g) on Biotage isolera one (Mobile phase A: 10 mM NH₄HCO₃ in water, B: ACN) to obtain N-((1r,3r)-3-(dimethylamino)cyclobutyl)-5-fluoro-2-((4-(6-((6-isobutyl-5,6,7,8-tetrahydro-1,6-naphthyridin-4-yl)oxy)-2-azaspiro[3.3]heptan-2-yl)pyrimidin-5-yl)oxy)-N-isopropylbenzamide as a solid.

Yield: 16.54%; ¹H NMR (400 MHz, DMSO-d₆): δ 8.28-8.25 (m, 1H), 8.18 (d, J=5.5 Hz, 1H), 7.71-7.64 (m, 1H), 7.31-7.06 (m, 3H), 6.67-6.63 (m, 1H), 4.74 (quin, J=6.6 Hz, 1H), 4.35-4.08 (m, 5H), 4.01-3.89 (m, 1H), 3.80-3.63 (m, 1H), 3.39 (br s, 2H), 2.98-2.90 (m, 1H), 2.87-2.74 (m, 5H), 2.70-2.62 (m, 2H), 2.29-2.21 (m, 4H), 2.17-2.07 (m, 1H), 2.06-1.92 (m, 6H), 1.90-1.79 (m, 2H), 1.48-1.29 (m, 3H), 1.06-0.85 (m, 9H); LCMS (Method A): Rt=2.53 min, m/z=672.5 [M+H]⁺; HPLC (Method D): Rt=6.25 min, 99.88%; Chiral SFC (Method Q): Rt=3.23 min, 100%.

### Example 78. N-((1r,3r)-3-(Dimethylamino)cyclobutyl)-5-fluoro-N-isopropyl-2-((4-(6-((6-isopropyl-5,6,7,8-tetrahydro-1,6-naphthyridin-4-yl)oxy)-2-azaspiro[3.3]heptan-2-yl)pyrimidin-5-yl)oxy)benzamide (Compound No. 78)

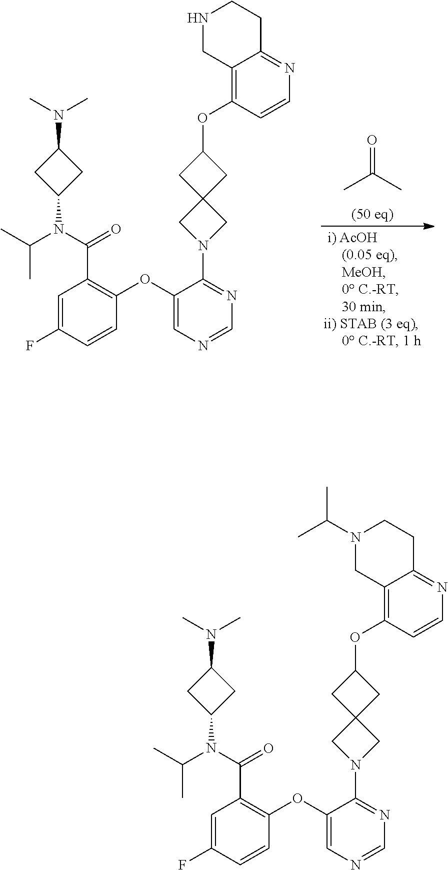

This compound was synthesized following the procedure described for the synthesis of Example 76, starting with 1 equivalent of N-((1r,3r)-3-(dimethylamino)cyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide and 50 equivalents of acetone. After completion, the crude was purified by reverse phase chromatography using a SiliCycle SiliaSep C18 column (80 g) on Biotage isolera one (Mobile phase A: 10 mM NH₄HCO₃ in water, B: ACN) to obtain N-((1r,3r)-3-(dimethylamino)cyclobutyl)-5-fluoro-N-isopropyl-2-((4-(6-((6-isopropyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 18.41%; ¹H NMR (400 MHz, DMSO-d₆): 8.29-8.25 (m, 1H), 8.16 (d, J=5.6 Hz, 1H), 7.77-7.63 (m, 1H), 7.32-7.22 (m, 2H), 7.17-7.06 (m, 1H), 6.67-6.62 (m, 1H), 4.75 (quin, J=6.6 Hz, 1H), 4.35-4.08 (m, 4H), 4.00-3.91 (m, 1H), 3.78-3.63 (m, 1H), 3.50 (s, 2H), 2.96-2.86 (m, 2H), 2.85-2.74 (m, 5H), 2.73-2.66 (m, 3H), 2.30-2.26 (m, 2H), 2.20-2.08 (m, 1H), 2.06-1.93 (m, 6H), 1.89-1.77 (m, 1H), 1.48-1.29 (m, 3H), 1.09-0.90 (m, 9H); LCMS (Method A): Rt=1.85 min, m/z=658.2 [M+H]⁺; HPLC (Method A): Rt=5.16 min, 99.78%; Chiral SFC (Method AL): Rt=1.02 min, 100%.

### Example 79. N-((1r,3r)-3-Acetamidocyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 79)

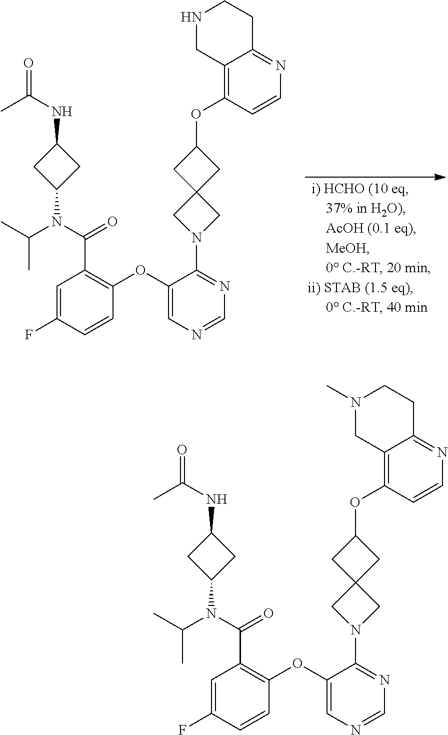 [SMILES: CC(=O)N[C@H]1C[C@H](N(C(=O)c2cc(F)ccc2Oc2cncnc2N2CC3(CC(Oc4ccnc5c4CNCC5)C3)C2)C(C)C)C1 ; CC(=O)N[C@H]1C[C@H](N(C(=O)c2cc(F)ccc2Oc2cncnc2N2CC3(CC(Oc4ccnc5c4CN(C)CC5)C3)C2)C(C)C)C1]

This compound was synthesized following the procedure described for the synthesis of Example 76, starting with 1 equivalent of N-((1r,3r)-3-acetamidocyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide, except that 10 equivalents of formaldehyde (37% in H₂O), 0.1 equivalent of AcOH, and 1.5 equivalents of STAB were used. After completion, the reaction was concentrated under reduced pressure. The crude product was purified by reverse phase chromatography using a SiliCycle SiliaSep C18 column (40 g) on Biotage isolera one (Mobile phase A: 10 mM NH₄HCO₃ in water, B: ACN) to afford N-((1r,3r)-3-acetamidocyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 71.7%; ¹H NMR (400 MHz, DMSO-d₆): δ 8.32-8.25 (m, 1H), 8.23-8.10 (m, 2H), 7.80-7.75 (m, 1H), 7.35-7.02 (m, 3H), 6.66 (d, J=5.6 Hz, 1H), 4.79-4.70 (m, 1H), 4.43-4.06 (m, 6H), 4.02-3.93 (m, 1H), 3.83-3.62 (m, 1H), 3.36 (s, 2H), 3.26-3.01 (m, 1H), 2.86-2.74 (m, 4H), 2.63 (br t, J=5.8 Hz, 2H), 2.36 (s, 3H), 2.31-2.22 (m, 2H), 2.12-1.86 (m, 2H), 1.83-1.73 (m, 3H), 1.50-0.91 (m, 6H); LCMS (Method A): Rt=1.50 min, m/z=644.2 [M+H]⁺; HPLC (Method A): Rt=4.50 min, 99.93%; Chiral SFC (Method AC): Rt=1.38 min, 100%.

### Example 80. N-(3-Acetamidobicyclo[1.1.1]pentan-1-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 80)

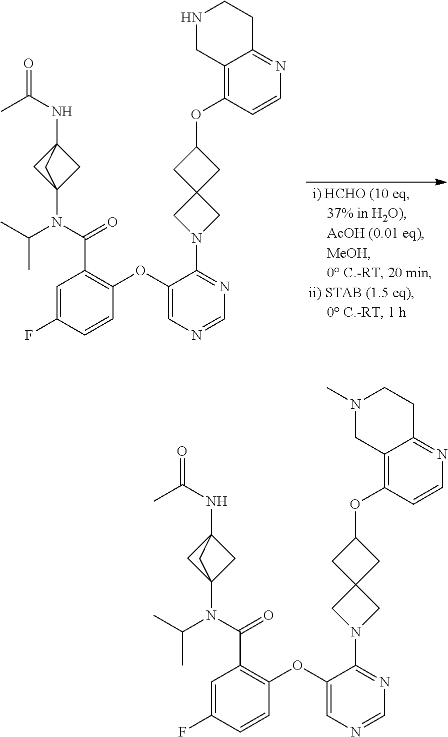 [SMILES: CC(=O)NC12CC(N(C(=O)c3cc(F)ccc3Oc3cncnc3N3CC4(CC(Oc5ccnc6c5CNCC6)C4)C3)C(C)C)(C1)C2 ; CC(=O)NC12CC(N(C(=O)c3cc(F)ccc3Oc3cncnc3N3CC4(CC(Oc5ccnc6c5CN(C)CC6)C4)C3)C(C)C)(C1)C2]

This compound was synthesized following the procedure described for the synthesis of Example 76, starting with 1 equivalent of N-(3-acetamidobicyclo[1.1.1]pentan-1-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide, except that 10 equivalents of formaldehyde (37% in H₂O), 0.1 equivalent of AcOH, and 1.5 equivalents of STAB were used. After filtration and concentration, the crude was purified by reverse phase chromatography using a SiliCycle SiliaSep C18 column (80 g) on Biotage isolera one (Mobile phase A: 10 mM NH₄HCO₃ in water, B: ACN) to obtain N-(3-acetamidobicyclo[1.1.1]pentan-1-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 83%; ¹H NMR (400 MHz, DMSO-d₆): δ 8.47-8.33 (m, 1H), 8.29 (s, 1H), 8.18 (d, J=5.6 Hz, 1H), 7.79-7.64 (m, 1H), 7.32-7.21 (m, 2H), 7.05 (dd, J=4.4, 9.1 Hz, 11H), 6.68 (d, J=4.9 Hz, 1H), 4.81-4.69 (m, 1H), 4.34-4.09 (m, 4H), 4.08-3.76 (m, 1H), 3.37 (s, 2H), 2.85-2.75 (m, 4H), 2.66-2.60 (m, 2H), 2.37 (s, 3H), 2.31-2.22 (m, 2H), 2.05-1.91 (m, 4H), 1.79-1.67 (m, 3H), 1.38-1.07 (m, 6H), two protons merged with solvent peaks; LCMS (Method A): Rt=1.58 min, m/z=656.3 [M+H]⁺; HPLC (Method A): Rt=4.58 min, 99.37%.

### Example 81. (S)-5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(1-methylpyrrolidin-3-yl)benzamide (Compound No. 81)

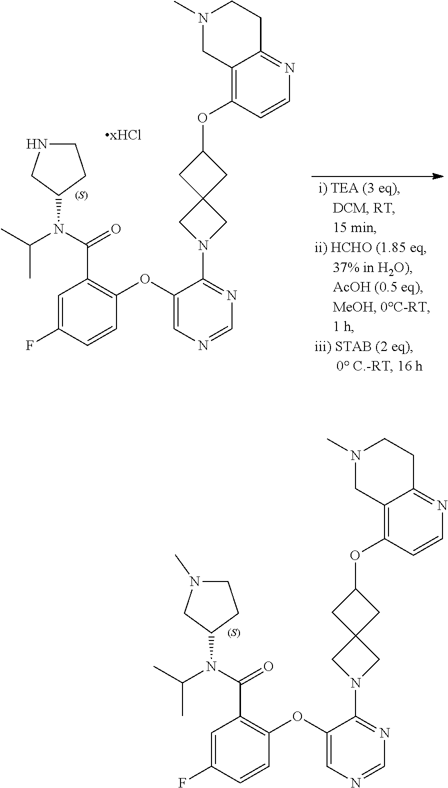

This compound was synthesized following the procedure described for the synthesis of Example 20, step 4, starting with 1 equivalent of (S)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(pyrrolidin-3-yl)benzamide xHCl, except that 1.85 equivalents of formaldehyde (37% in H₂O), 3 equivalents of TEA, 0.5 equivalent of AcOH, and 2 equivalents of STAB were used. The crude was purified by reverse phase chromatography using a SiliCycle SiliaSep C18 column (60 g) on Biotage isolera one (Mobile phase A: 10 mM NH₄HCO₃ in water, B: ACN), then by Prep HPLC (Method A)¹HNMR of this compound showed impurities, therefore, the solid was re-purified by reverse phase chromatography using a SiliCycle SiliaSep C18 column (60 g) on Biotage isolera one (Mobile phase A: 10 mM NH₄HCO₃ in water, B: ACN). The pure fractions were concentrated under reduced pressure and lyophilized to afford (S)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(1-methylpyrrolidin-3-yl)benzamide as a solid.

Yield: 10.01%; ¹H NMR (400 MHz, DMSO-d₆): δ 8.30-8.25 (m, 1H), 8.18 (d, J=5.6 Hz, 1H), 7.79-7.72 (m, 1H), 7.31-7.16 (m, 2H), 7.12-7.02 (m, 1H), 6.70-6.62 (m, 1H), 4.79-4.70 (m, 1H), 4.34-4.03 (m, 4H), 3.89-3.65 (m, 2H), 3.36 (s, 2H), 2.84-2.73 (m, 5H), 2.67-2.60 (m, 3H), 2.36 (s, 3H), 2.30-2.12 (m, 5H), 2.00-1.72 (m, 11H), 1.55-0.91 (m, 6H), three protons merged with solvent peaks; LCMS (Method C): Rt=1.12 min, m/z=616.3 [M+H]⁺; HPLC (Method D): Rt=4.37 min, 98.89%; Chiral SFC (Method Q): Rt=4.01 min, 100%.

### Example 82. (S)-5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(1-(methylsulfonyl)pyrrolidin-3-yl)benzamide (Compound No. 82)

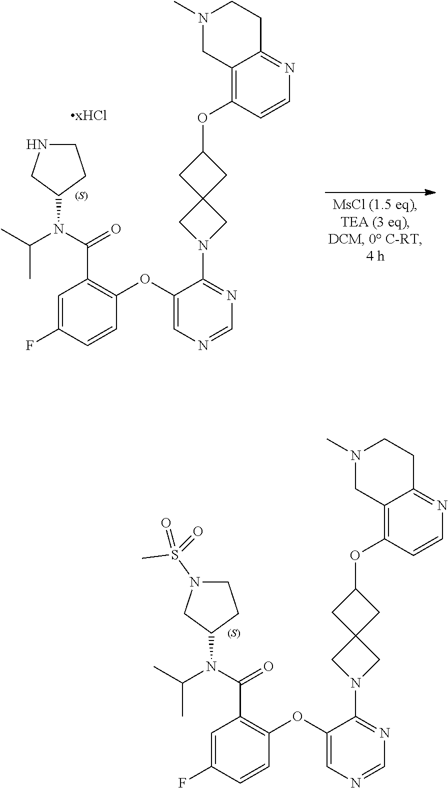

This compound was synthesized following the procedure described for the synthesis of Example 68, step 1, starting with 1 equivalent of (S)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(pyrrolidin-3-yl)benzamide xHCl and 1.5 equivalents of methanesulfonyl chloride were used. The crude was purified by reverse phase chromatography using a SiliCycle SiliaSep C18 column (40 g) on Biotage isolera one (Mobile phase A: 10 mM NH₄HCO₃ in water, B: ACN) to obtain (S)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(1-(methylsulfonyl)pyrrolidin-3-yl)benzamide as a solid.

Yield: 32.5%; ¹H NMR (400 MHz, DMSO-d₆): δ 8.31-8.28 (m, 1H), 8.20-8.16 (m, 1H), 7.81-7.76 (m, 1H), 7.41-7.32 (m, 1H), 7.30-7.22 (m, 1H), 7.10-7.00 (m, 1H), 6.66 (d, J=5.6 Hz, 1H), 4.80-4.69 (m, 1H), 4.26-4.09 (m, 5H), 3.79-3.65 (m, 11H), 3.61-3.47 (m, 1H), 3.36 (s, 2H), 2.95-2.85 (m, 3H), 2.84-2.73 (m, 4H), 2.62 (br t, J=5.8 Hz, 2H), 2.36 (s, 3H), 2.30-2.20 (m, 2H), 2.10-1.96 (m, 1H), 1.52-0.97 (m, 6H), four protons merged in solvent peaks; LCMS (Method B): Rt=1.03 min, m/z=680.2 [M+H]⁺; HPLC (Method A): Rt=4.93 min, 97.21%; Chiral SFC (Method R): Rt=1.45 min, 96.99%.

### Example 83. (S)—N-(1-Acetylpyrrolidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 83)

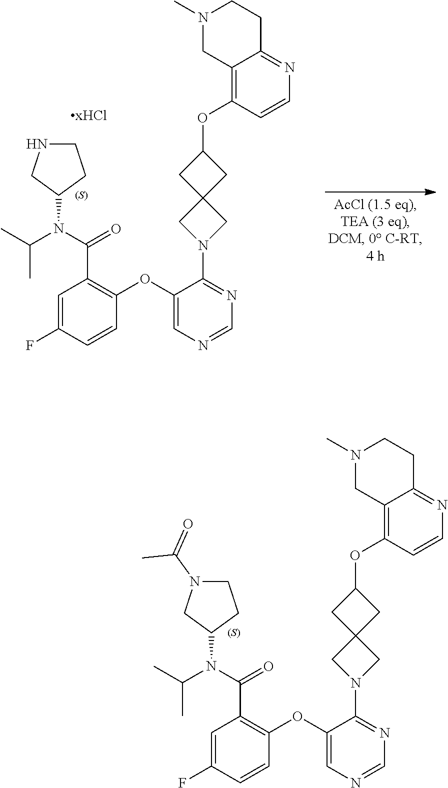

This compound was synthesized following the procedure described for the synthesis of Example 68, step 1, starting with 1 equivalent of (S)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(pyrrolidin-3-yl)benzamide xHCl and 1.5 equivalents of acetyl chloride. The crude was purified by reverse phase chromatography using a SiliCycle SiliaSep C18 column (40 g) on Biotage isolera one (Mobile phase A: 10 mM NH₄HCO₃ in water, B: ACN) to obtain (S)—N-(1-acetylpyrrolidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 54%; ¹H NMR (400 MHz, DMSO-d₆): δ 8.33-8.27 (m, 1H), 8.18 (d, J=5.6 Hz, 1H), 7.83-7.76 (m, 1H), 7.37-7.21 (m, 2H), 7.10-6.97 (m, 1H), 6.72-6.62 (m, 1H), 4.80-4.68 (m, 1H), 4.33-3.93 (m, 5H), 3.82-3.40 (m, 4H), 3.36 (s, 2H), 3.27-3.18 (m, 1H), 2.87-2.74 (m, 4H), 2.63 (br t, J=5.6 Hz, 2H), 2.36 (s, 3H), 2.31-2.19 (m, 2H), 2.01-1.81 (m, 4H), 1.48-0.99 (m, 6H), one proton merged with solvent peaks; LCMS (Method B): Rt=0.99 min, m/z=644.2 [M+H]⁺; HPLC (Method A): Rt=4.49 min, 98.07%; Chiral SFC (Method N): Rt=1.58 min, 100%.

### Example 84. N-(1-Cyclobutylazetidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 84)

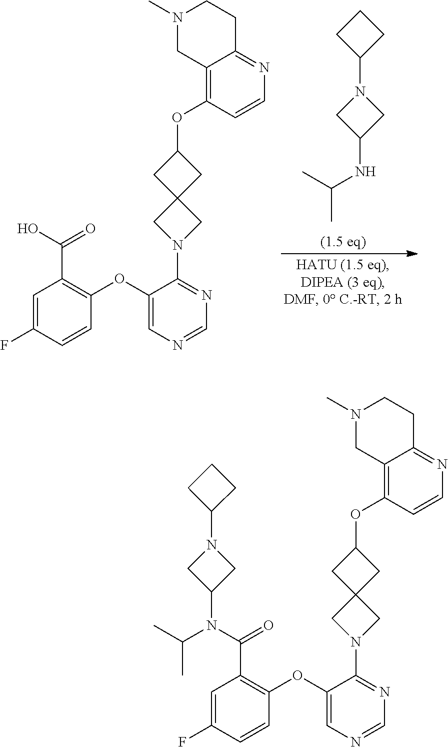 [SMILES: CN1CCc2nccc(OC3CC4(C3)CN(c3ncncc3Oc3ccc(F)cc3C(=O)O)C4)c2C1 ; CC(C)NC1CN(C2CCC2)C1 ; CC(C)N(C(=O)c1cc(F)ccc1Oc1cncnc1N1CC2(CC(Oc3ccnc4c3CN(C)CC4)C2)C1)C1CN(C2CCC2)C1]

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.5 equivalent of 1-cyclobutyl-N-isopropylazetidin-3-amine. After work-up, the crude compound was purified by reverse phase chromatography using a SiliCycle SiliaSep C18 column (120 g) on Biotage isolera one (Mobile phase A: 10 mM NH₄HCO₃ in water, B: ACN), then by Prep HPLC (Method A). The pure fractions were concentrated under reduced pressure to afford N-(1-cyclobutylazetidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 26.0%; ¹H NMR (400 MHz, DMSO-d₆): δ 8.32-8.25 (m, 1H), 8.18 (d, J=5.6 Hz, 1H), 7.81-7.68 (m, 1H), 7.35-7.16 (m, 2H), 7.12-7.01 (m, 1H), 6.66 (d, J=5.6 Hz, 1H), 4.75 (quin, J=6.6 Hz, 1H), 4.34-3.99 (m, 6H), 3.81-3.64 (m, 1H), 3.36 (s, 2H), 3.25-3.16 (m, 1H), 3.14-2.99 (m, 2H), 2.97-2.90 (m, 1H), 2.84-2.76 (m, 4H), 2.63 (br t, 0.1=5.8 Hz, 2H), 2.36 (s, 3H), 2.25 (br dd, J=6.1, 11.9 Hz, 2H), 1.89-1.72 (m, 3H), 1.70-1.52 (m, 3H), 1.49-0.96 (m, 6H); LCMS (Method A): Rt=1.77 min, m/z=642.2 [M+H]⁺; HPLC (Method B): Rt=4.95 min, 98.55%.

### Example 85. N-((1r,3r)-3-(Dimethylamino)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 85)

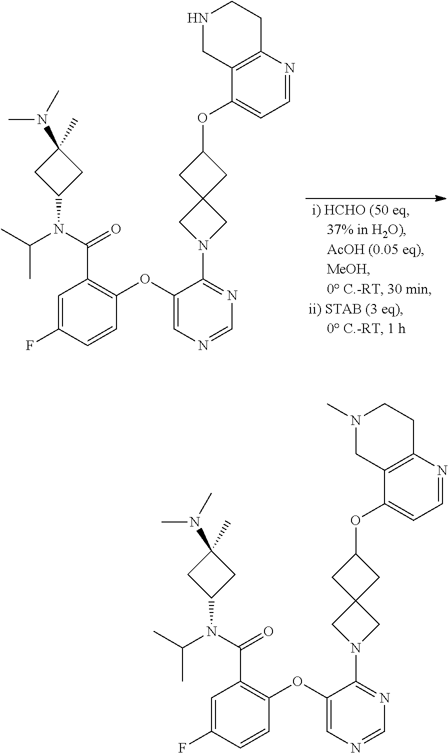

This compound was synthesized following the procedure described for the synthesis of Example 76, starting with 1 equivalent of N-((1r,3r)-3-(dimethylamino)-3-methylcyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide. After completion, the crude was purified by reverse phase chromatography using a SiliCycle SiliaSep C18 column (80 g) on Biotage isolera one (Mobile phase A: 10 mM NH₄HCO₃ in water, B: ACN) and then by Pep HPLC (Method K) to obtain N-((1r,3r)-3-(dimethylamino)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 50.4%; ¹H NMR (400 MHz, DMSO-d₆): δ 8.30-8.24 (d, J=5.3 Hz, 1H), 8.18 (d, J=5.5 Hz, 1H), 7.77-7.59 (m, 1H), 7.31-7.22 (m, 1H), 7.18-7.04 (m, 2H), 6.69-6.62 (m, 1H), 4.75 (quin, J=6.6 Hz, 1H), 4.35-3.94 (m, 5H), 3.86-3.63 (m, 2H), 3.36 (s, 2H), 2.84-2.75 (m, 5H), 2.63 (t, J=5.8 Hz, 2H), 2.36 (s, 3H), 2.30-2.21 (m, 2H), 2.18-1.88 (m, 6H), 1.96-1.87 (m, 1H), 1.80-1.68 (m, 1H), 1.47-1.26 (m, 3H), 1.15-0.90 (m, 6H); LCMS (Method H): Rt=0.94 min, m/z=644.4 [M+H]⁺; HPLC (Method A): Rt=4.84 min, 99.74%; Chiral SFC (Method L): Rt=1.85 min, 100%.

### Example 86. N-((1s,3s)-3-(Dimethylamino)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 86)

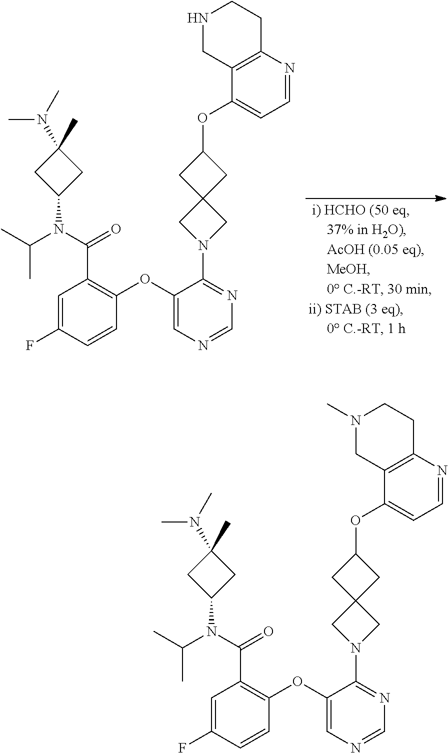 [SMILES: CC(C)N(C(=O)c1cc(F)ccc1Oc1cncnc1N1CC2(CC(Oc3ccnc4c3CNCC4)C2)C1)[C@H]1C[C@@](C)(N(C)C)C1 ; CC(C)N(C(=O)c1cc(F)ccc1Oc1cncnc1N1CC2(CC(Oc3ccnc4c3CN(C)CC4)C2)C1)[C@H]1C[C@@](C)(N(C)C)C1]

This compound was synthesized following the procedure described for the synthesis of Example 76, starting with 1 equivalent of N-((1s,3s)-3-(dimethylamino)-3-methylcyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide. After completion, the crude was purified by reverse phase chromatography using a SiliCycle SiliaSep C18 column (80 g) on Biotage isolera one (Mobile phase A: 10 mM NH₄HCO₃ in water, B: ACN) and then by Prep HPLC (Method K) to obtain N-((1s,3s)-3-(dimethylamino)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 25.8%; ¹H NMR (400 MHz, DMSO-d₆): δ 8.30-8.25 (m, 1H), 8.18 (d, J=5.6 Hz, 1H), 7.78-7.67 (m, 1H), 7.31-7.22 (m, 1H), 7.18-7.03 (m, 2H), 6.65 (d, J=5.8 Hz, 1H), 4.75 (quin, J=6.6 Hz, 1H), 4.37-4.06 (m, 4H), 3.87-3.66 (m, 2H), 3.36 (s, 2H), 2.85-2.75 (m, 4H), 2.66-2.59 (m, 2H), 2.36 (s, 3H), 2.29-2.21 (m, 2H), 2.05-1.84 (m, 9H), 1.75-1.67 (m, 1H), 1.47-0.80 (m, 9H); LCMS (Method C): Rt=1.39 min, m/z=644.3 [M+H]⁺; HPLC (Method A): Rt=4.83 min, 99.03%; Chiral SFC (Method P): Rt=3.06 min, 98.95%.

### Example 87. N-((1r,3r)-3-(Dimethylamino)cyclobutyl)-2-((4-(6-((6-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide. (Compound No. 87)

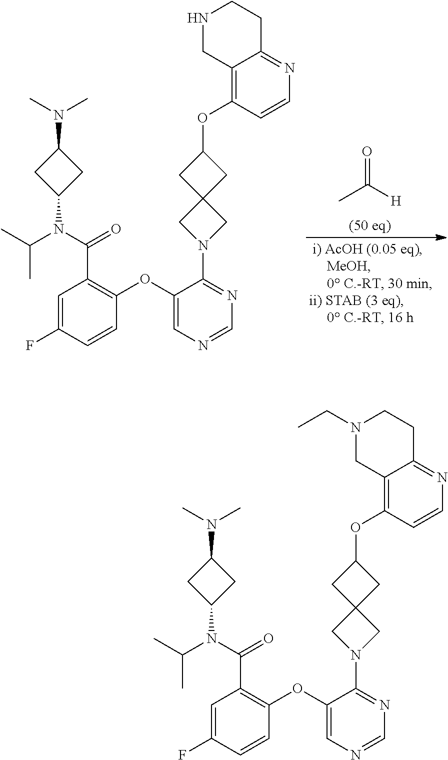

This compound was synthesized following the procedure described for the synthesis of Example 76, starting with 1 equivalent of N-((1r,3r)-3-(dimethylamino)cyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide and 50 equivalents of acetaldehyde. After completion, the crude was purified by reverse phase chromatography using a SiliCycle SiliaSep C18 column (80 g) on Biotage isolera one (Mobile phase A: 10 mM NH₄HCO₃ in water, B: ACN), then by Prep HPLC (Method A) to obtain N-((1r,3r)-3-(dimethylamino)cyclobutyl)-2-((4-(6-((6-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide as a solid.

Yield: 10.99%; 1H NMR (400 MHz, DMSO-d₆): δ 8.30-8.25 (m, 1H), 8.18 (d, J=5.5 Hz, 1H), 7.76-7.63 (m, 1H), 7.31-7.22 (m, 2H), 7.16-7.06 (m, 1H), 6.68-6.63 (m, 1H), 4.79-4.70 (m, 1H), 4.33-4.05 (m, 5H), 4.02-3.90 (m, 1H), 3.79-3.63 (m, 1H), 3.41 (s, 2H), 2.98-2.84 (m, 1H), 2.84-2.74 (m, 5H), 2.70-2.66 (m, 2H), 2.29-2.22 (m, 2H), 2.20-1.79 (m, 8H), 1.50-1.28 (m, 3H), 1.08 (t, J=7.1 Hz, 3H), 1.05-0.90 (m, 3H), two protons merged with solvent peaks; LCMS (Method C): Rt=1.16 min, m/z=644.3 [M+H]⁺; HPLC (Method D): Rt=4.92 min, 99.82%; Chiral SFC (Method AD): Rt=2.90 min, 100%.

### Example 88. N-((1R,3S)-3-(Dimethylamino)cyclopentyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 88)

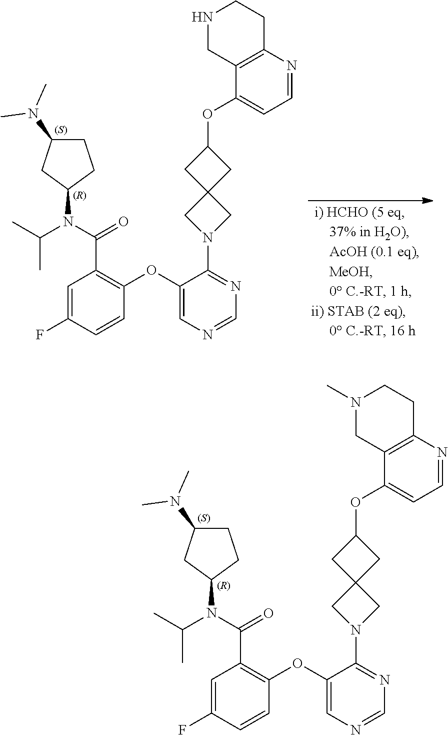

This compound was synthesized following the procedure described for the synthesis of Example 76, starting with 1 equivalent of N-((1R,3S)-3-(dimethylamino)cyclopentyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide, except that 5 equivalents of formaldehyde (37% in H₂O), 0.1 equivalent of AcOH, and 2 equivalents of STAB were used. After workup, the crude was purified by reverse phase chromatography using a SiliCycle SiliaSep C18 column (40 g) on Biotage isolera one (Mobile phase A: 10 mM NH₄HCO₃ in water, B: ACN), then by Prep HPLC (Method A). The fractions were concentrated under reduced pressure and lyophilized to obtain N-((1R,3S)-3-(dimethylamino)cyclopentyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (26.9% yield) as a solid. ¹H NMR (400 MHz, DMSO-d₆): δ 8.30-8.24 (m, 1H), 8.18 (d, J=5.6 Hz, 1H), 7.77-7.63 (m, 1H), 7.29-7.18 (m, 2H), 7.15-7.03 (m, 1H), 6.68-6.62 (m, 1H), 4.75 (quin, J=6.4 Hz, 1H), 4.34-4.09 (m, 4H), 3.74-3.61 (m, 2H), 3.36 (br s, 2H), 2.84-2.74 (m, 4H), 2.63 (br t, J=5.8 Hz, 2H), 2.36 (s, 3H), 2.30-2.21 (m, 2H), 2.16-2.00 (m, 7H), 1.84-1.59 (m, 4H), 1.49-0.93 (m, 6H), two protons merged with solvent peaks; LCMS (Method C): Rt=1.36 min, m/z=644.3 [M+H]⁺; HPLC (Method C): Rt=8.01 min, 99.89%; Chiral SFC (Method T): Rt=3.11 min, 100%.

### Example 89. N-((1S,3R)-3-(Dimethylamino)cyclopentyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 89)

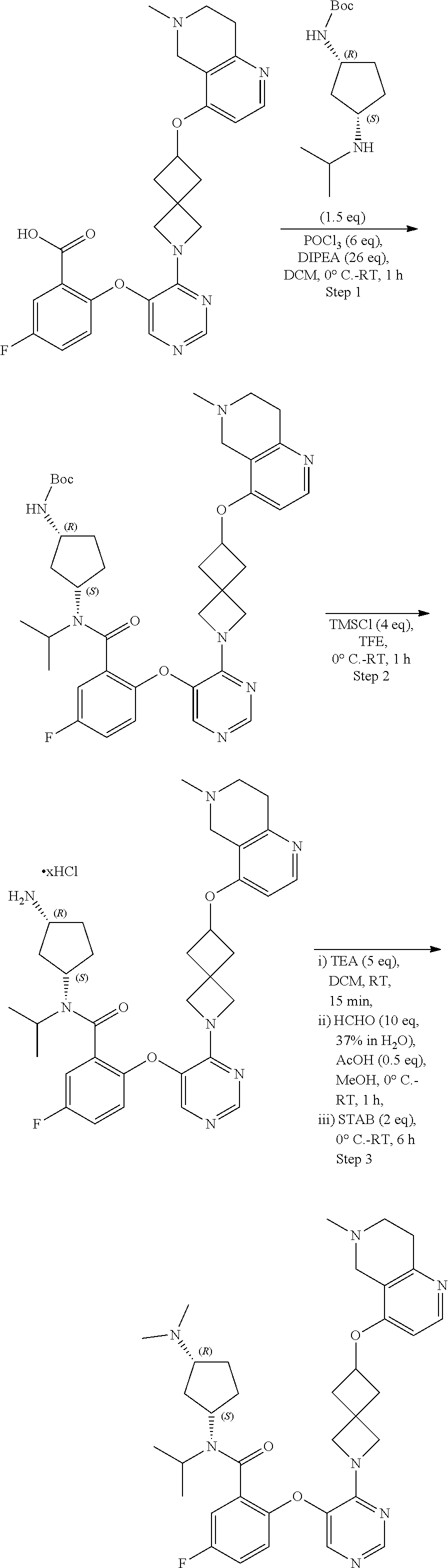

### Step 1. tert-Butyl ((1R,3S)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclopentyl)carbamate

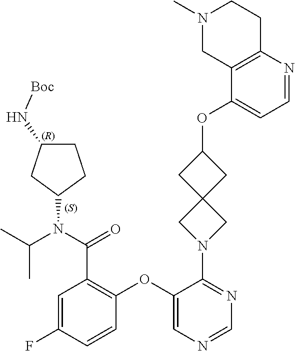

This compound was synthesized following the procedure described for the synthesis of Example 20, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.5 equivalent of tert-butyl ((1R,3,S)-3-(isopropylamino)cyclopentyl)carbamate. After work-up, the crude was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure to afford tert-butyl ((1R,3S)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclopentyl)carbamate as a solid.

Yield: 35.3%; LCMS (Method B): Rt=1.26 min, m/z=716.3 [M+H]⁺, 96.78%.

### Step 2. N-((1S,3R)-3-Aminocyclopentyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl

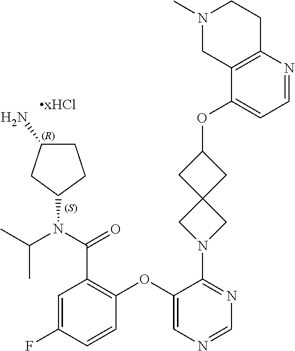

This compound was synthesized following the procedure described for the synthesis of Example 20, step 3, starting with 1 equivalent of tert-butyl ((1R,3S)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclopentyl)carbamate. After completion, the reaction was concentrated under reduced pressure to obtain crude N-((1S,3R)-3-aminocyclopentyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl. This material was used without further purification.

Yield: Quantitative; LCMS (Method B): Rt=0.81-0.86 min, m/z=616.4 [M+H]⁺, 97.19%.

### Step 3. N-((1S,3R)-3-(Dimethylamino)cyclopentyl)-5-fluoro-N-isopropyl-2-((4-(6-((6-methyl-5,6,7,8-tetrahydro-1. 6-naphthyridin-4-yl)oxy)-2-azaspiro[3.3]heptan-2-yl)pyrimidin-5-yl)oxy)benzamide

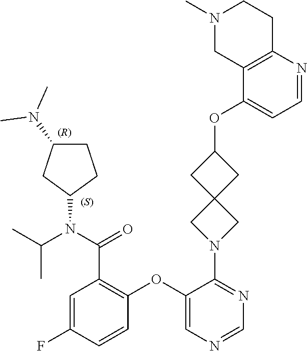

This compound was synthesized following the procedure described for the synthesis of Example 20, step 4, starting with 1 equivalent of N-((1S,3R)-3-aminocyclopentyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl, except that 5 equivalents of TEA, 0.5 equivalent of AcOH and 3 equivalents of ST AB were used. After completion, the reaction was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure and lyophilized to afford N-((1S,3R)-3-(dimethylamino)cyclopentyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 22.98%; ^{l}H NMR (400 MHz, DMSO-d₆): δ 8.30-8.25 (m, 1H), 8.18 (d, J=5.6 Hz, 1H), 7.77-7.67 (m, 1H), 7.29-7.15 (m, 2H), 7.14-7.02 (m, 1H), 6.67-6.62 (m, 1H), 4.75 (quin, J=6.5 Hz, 1H), 4.34-4.09 (m, 4H), 3.87-3.42 (m, 2H), 3.36 (s, 2H), 2.85-2.73 (m, 4H), 2.62 (br t, J=5.8 Hz, 2H), 2.36 (s, 3H), 2.29-2.21 (m, 3H), 2.17-2.00 (m, 6H), 1.86-1.60 (m, 4H), 1.48-0.93 (m, 6H), two protons merged with solvent peaks; LCMS (Method C): Rt=1.39 min, m/z=644.5 [M+H]⁺; HPLC (Method E): Rt=8.68 min, 98.66%; Chiral SFC (Method T): Rt=2.72 min, 100%.

### Example 90. N-(3-(Dimethylamino)bicyclo[1.1.1]pentan-1-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 90)

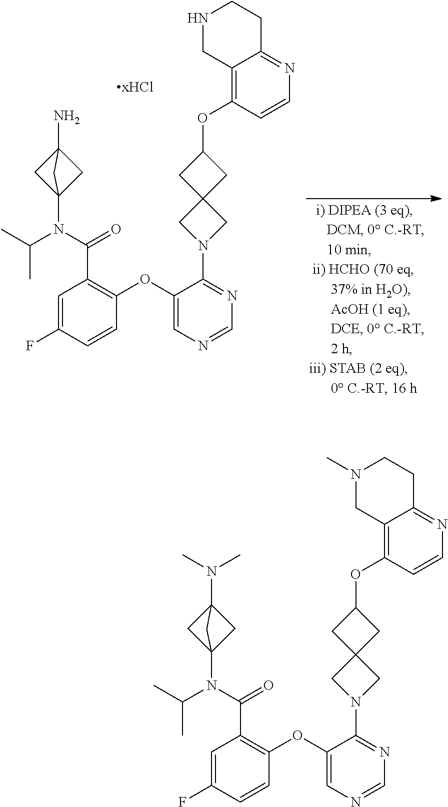 [SMILES: CC(C)N(C(=O)c1cc(F)ccc1Oc1cncnc1N1CC2(CC(Oc3ccnc4c3CNCC4)C2)C1)C12CC(N)(C1)C2 ; CC(C)N(C(=O)c1cc(F)ccc1Oc1cncnc1N1CC2(CC(Oc3ccnc4c3CN(C)CC4)C2)C1)C12CC(N(C)C)(C1)C2]

This compound was synthesized following the procedure described for the synthesis of Example 20, step 4, starting with 1 equivalent of N-(3-aminobicyclo[1.1.1]pentan-1-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl, except that, 3 equivalents of DIPEA, 70 equivalents of formaldehyde (37% in H₂O), 1 equivalent of acetic acid and 2 equivalents of STAB were used. After filtration and concentration, the crude was purified by Prep HPLC (Method A) to obtain N-(3-(dimethylamino)bicyclo[1.1.1]pentan-1-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 11.67%; ¹H NMR (400 MHz, DMSO-d₆): δ 8.29 (br s, 1H), 8.18 (d, J=5.6 Hz, 1H), 7.79-7.66 (m, 1H), 7.30-7.19 (m, 2H), 7.05 (dd, J=4.3, 9.0 Hz, 1H), 6.66 (d, J=5.6 Hz, 1H), 4.81-4.67 (m, 1H), 4.32-4.08 (m, 4H), 4.06-3.77 (m, 1H), 3.37 (s, 2H), 2.86-2.74 (m, 4H), 2.63 (t, J=5.5 Hz, 2H), 2.37 (s, 3H), 2.30-2.18 (m, 4H), 2.11 (s, 3H), 2.01 (s, 3H), 1.79-1.63 (m, 3H), 1.40-1.07 (m, 6H), one proton merged with solvent peaks; LCMS (Method A): Rt=1.73 min, m/z=642.3 [M+H]⁺; HPLC (Method A): Rt=5.05 min, 98.61%.

### Example 91. N-(3-(Dimethylamino)propyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 91)

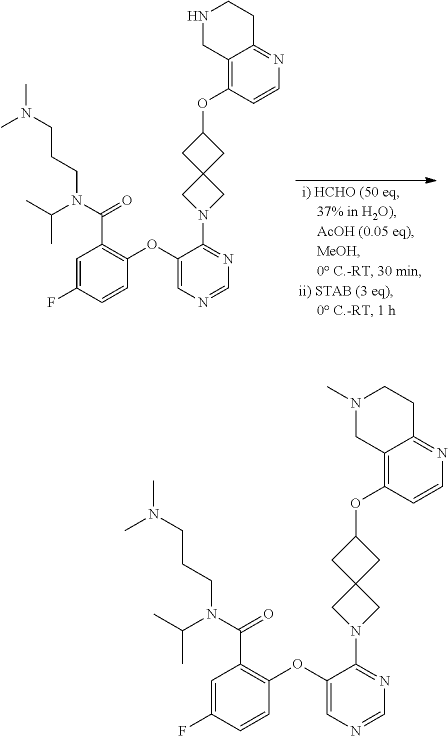 [SMILES: CC(C)N(CCCN(C)C)C(=O)c1cc(F)ccc1Oc1cncnc1N1CC2(CC(Oc3ccnc4c3CNCC4)C2)C1 ; CC(C)N(CCCN(C)C)C(=O)c1cc(F)ccc1Oc1cncnc1N1CC2(CC(Oc3ccnc4c3CN(C)CC4)C2)C1]

This compound was synthesized following the procedure described for the synthesis of Example 76, starting with 1 equivalent of N-(3-(dimethylamino)propyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide After completion, the crude was purified by reverse phase chromatography using a SiliCycle SiliaSep C18 column (80 g) on Biotage isolera one (Mobile phase A: 10 mM NH₄HCO₃ in water, B: ACN) to obtain N-(3-(dimethylamino)propyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 62.1%; ¹H NMR (400 MHz, DMSO-d₆): δ 8.29-8.28 (m, 1H), 8.18 (d, J=5.6 Hz, 1H), 7.74-7.68 (m, 1H), 7.35-7.23 (m, 2H), 7.05 (dd, J=4.3, 8.9 Hz, 1H), 6.66 (d, J=5.6 Hz, 1H), 4.75 (quin, J=6.5 Hz, 1H), 4.31-4.06 (m, 4H), 3.81-3.71 (m, 1H), 3.36 (s, 2H), 3.18-3.08 (m, 1H), 2.84-2.74 (m, 4H), 2.63 (t, J=5.8 Hz, 2H), 2.36 (s, 3H), 2.29-2.18 (m, 4H), 2.12-1.88 (m, 7H), 1.78-1.50 (m, 2H), 1.23-1.00 (m, 6H); LCMS (Method H): Rt=0.78 min, m/z=618.4 [M+H]⁺; HPLC (Method A): Rt=4.48 min, 99.22%.

### Example 92. N-(2-(Dimethylamino)ethyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 92)

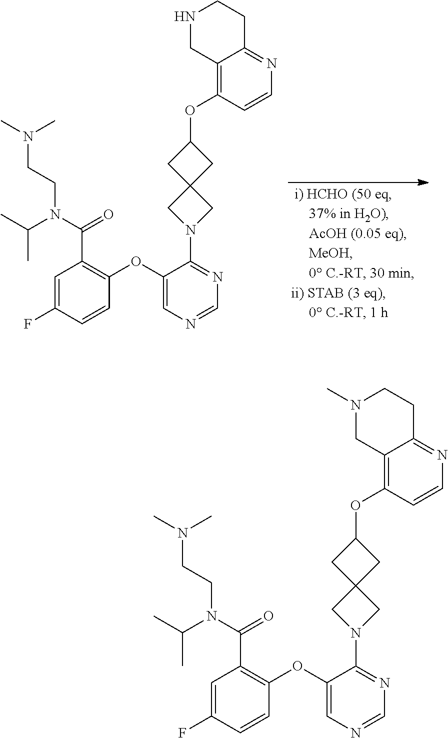 [SMILES: CC(C)N(CCN(C)C)C(=O)c1cc(F)ccc1Oc1cncnc1N1CC2(CC(Oc3ccnc4c3CNCC4)C2)C1 ; CC(C)N(CCN(C)C)C(=O)c1cc(F)ccc1Oc1cncnc1N1CC2(CC(Oc3ccnc4c3CN(C)CC4)C2)C1]

This compound was synthesized following the procedure described for the synthesis of Example 76, starting with 1 equivalent of N-(2-(dimethylamino)ethyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide. After completion, the reaction was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain N-(2-(dimethylamino)ethyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 67.2%; ¹H NMR (400 MHz, DMSO-d₆): δ 8.30-8.27 (m, 1H), 8.18 (d, J=5.6 Hz, 1H), 7.75-7.63 (m, 1H), 7.35-7.23 (m, 2H), 7.11-7.01 (m, 1H), 6.66 (d, J=5.6 Hz, 1H), 4.80-4.71 (m, 1H), 4.45-4.06 (m, 4H), 3.80-3.69 (m, 1H), 3.47-3.36 (m, 3H), 3.29-3.13 (m, 1H), 2.84-2.75 (m, 4H), 2.62 (br t, J=5.7 Hz, 2H), 2.47-2.40 (m, 1H), 2.36 (s, 3H), 2.30-2.22 (m, 3H), 2.20-1.91 (m, 6H), 1.21-1.00 (m, 6H); LCMS (Method H): Rt=1.57 min, m/z=604.3 [M+H]⁺, 98.31%.

### Example 93. (S)-5-Fluoro-N-isopropyl-2-((5-(6-((6-methyl-5,6,7,8-tetrahydro-1,6-naphthyridin-4-yl)oxy)-2-azaspiro[3.3]heptan-2-yl)-1,2,4-triazin-6-yl)oxy)-N-(1-methylpyrrolidin-3-yl)benzamide (Compound No. 93)

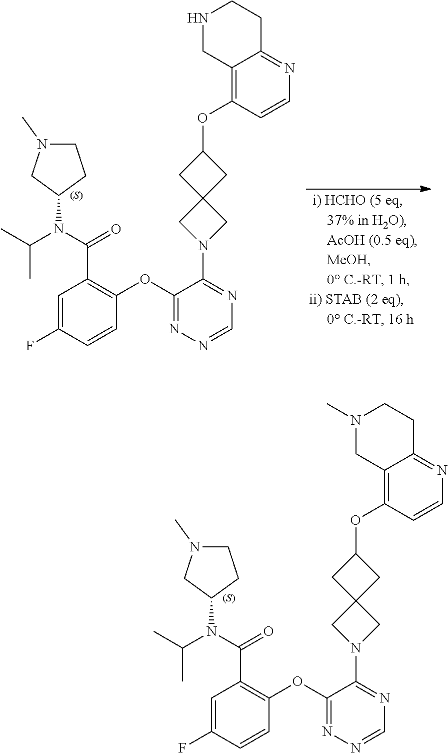

This compound was synthesized following the procedure described for the synthesis of Example 76, starting with 1 equivalent of (S)-5-fluoro-N-isopropyl-N-(1-methylpyrrolidin-3-yl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide, except that 5 equivalents of formaldehyde (37% in H₂O), 0.5 equivalent of AcOH, and 2 equivalents of STAB were used. After workup, the crude compound was purified by reverse phase chromatography using a SiliCycle SiliaSep C18 column (40 g) on Biotage isolera one (Mobile phase A: 10 mM NH₄HCO₃ in water, B: ACN), then by Prep HPLC (Method A). The pure fractions were concentrated under reduced pressure and lyophilized to obtain (S)-5-fluoro-N-isopropyl-2-((5-(6-((6-methyl-5,6,7,8-tetrahydro-1,6-naphthyridin-4-yl)oxy)-2-azaspiro[3.3]heptan-2-yl)-1,2,4-triazin-6-yl)oxy)-N-(1-methylpyrrolidin-3-yl)benzamide (23.04% yield) as a solid. ¹H NMR (400 MHz, DMSO-d₆): δ 8.49-8.44 (m, 1H), 8.20 (d, J=5.6 Hz, 1H), 7.47-7.40 (m, 1H), 7.39-7.20 (m, 2H), 6.71-6.64 (m, 1H), 4.88-4.74 (m, 1H), 4.72-4.51 (m, 2H), 4.30-4.09 (m, 2H), 4.02-3.90 (m, 1H), 3.84-3.66 (m, 1H), 3.65-3.55 (m, 1H), 3.38 (br s, 2H), 2.89-2.78 (m, 4H), 2.65-2.59 (m, 3H), 2.41-2.35 (m, 4H), 2.31-2.04 (m, 5H), 2.01-1.71 (m, 2H), 1.52-0.61 (m, 6H), one proton merged with solvent peaks; LCMS (Method C): Rt=1.32 min, m/z=617.3 [M+H]⁺; HPLC (Method A): Rt=4.24 min, 99.94%, Chiral SFC (Method V): Rt=6.66 min, 100%.

### Example 94. (R)-5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(1-methylpiperidin-3-yl)benzamide (Compound No. 94)

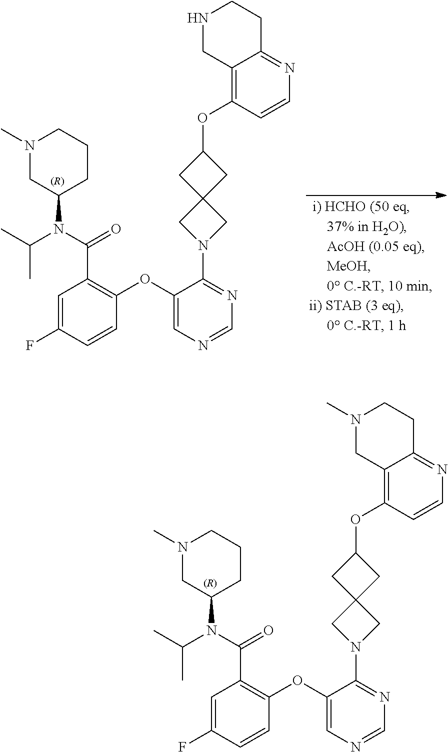

This compound was synthesized following the procedure described for the synthesis of Example 76, starting with 1 equivalent of (R)-5-fluoro-N-isopropyl-N-(1-methylpiperidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide. After completion, the crude was purified by reverse phase chromatography using a SiliCycle SiliaSep C18 column (80 g) on Biotage isolera one (Mobile phase A: 10 mM NH₄HCO₃ in water, B: ACN) to obtain (R)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(1-methylpiperidin-3-yl)benzamide as a solid.

Yield: 52.7%; ¹H NMR (400 MHz, DMSO-d₆): δ 8.32-8.26 (m, 1H), 8.18 (d, J=5.8 Hz, 1H), 7.77-7.63 (m, 1H), 7.32-7.19 (m, 2H), 7.14-7.00 (m, 1H), 6.66 (d, J=5.6 Hz, 1H), 4.79-4.71 (m, 1H), 4.34-4.08 (m, 4H), 3.77-3.53 (m, 1H), 3.37 (br s, 2H), 3.28-3.17 (m, 1H), 2.86-2.74 (m, 5H), 2.69-2.62 (m, 2H), 2.37 (s, 3H), 2.30-1.91 (m, 6H), 1.87-1.48 (m, 4H), 1.46-1.31 (m, 3H), 1.14-0.92 (m, 3H), two protons merged with solvent peaks; LCMS (Method A): Rt=1.70 min, m/z=630.3 [M+H]⁺; HPLC (Method A): Rt=4.77 min, 99.41%; Chiral SFC (Method C): Rt=3.63 min, 100%.

### Example 95. (D1)-5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(3-(1-methylazetidin-3-yl)cyclobutyl)benzamide (Compound No. 95)

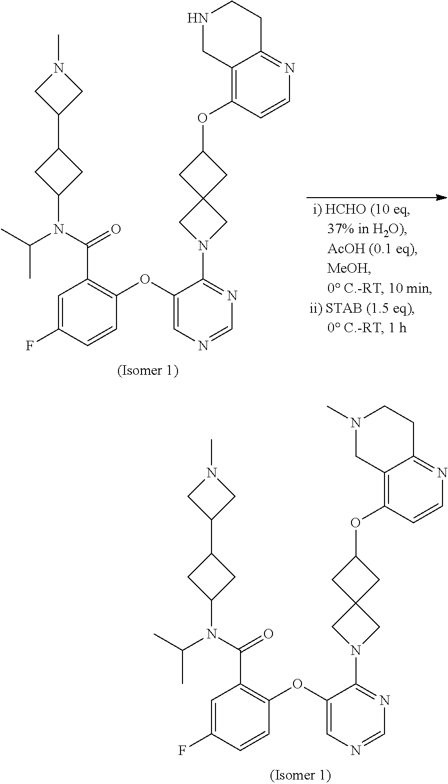 [SMILES: CC(C)N(C(=O)c1cc(F)ccc1Oc1cncnc1N1CC2(CC(Oc3ccnc4c3CNCC4)C2)C1)C1CC(C2CN(C)C2)C1 ; CC(C)N(C(=O)c1cc(F)ccc1Oc1cncnc1N1CC2(CC(Oc3ccnc4c3CN(C)CC4)C2)C1)C1CC(C2CN(C)C2)C1]

This compound was synthesized following the procedure described for the synthesis of Example 76, starting with 1 equivalent of (D1)-5-fluoro-N-isopropyl-N-(3-(1-methylazetidin-3-yl)cyclobutyl)-2-((4-(6-((5,6,7,8-tetrahydro-1,6-naphthyridin-4-yl)oxy)-2-azaspiro[3.3]heptan-2-yl)pyrimidin-5-yl)oxy)benzamide, except that 10 equivalents of formaldehyde (37% H₂O), 0.1 equivalent of AcOH, and 1.5 equivalents of STAB were used. After work-up, the crude product was purified by reverse phase chromatography using a SiliCycle SiliaSep C18 column (40 g) on Biotage isolera one (Mobile phase A: 10 mM NH₄HCO₃ in water, B: ACN) to afford (D/)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(3-(1-methylazetidin-3-yl)cyclobutyl)benzamide as a solid.

Yield: 56.8%; ¹H NMR (400 MHz, DMSO-d₆): δ 8.33-8.24 (m, 1H), 8.18 (d, J=5.6 Hz, 1H), 7.76 (d, J=15.3 Hz, 1H), 7.35-7.01 (m, 3H), 6.66 (br d, J=2.5 Hz, 1H), 4.74 (quin, J=6.6 Hz, 1H), 4.43-4.04 (m, 4H), 3.90-3.65 (m, 2H), 3.36 (s, 2H), 3.12 (br t, J=6.9 Hz, 2H), 2.87-2.74 (m, 6H), 2.66-2.60 (m, 3H), 2.36 (s, 3H), 2.30-2.14 (m, 3H), 2.10 (s, 3H), 2.06-1.96 (m, 1H), 1.94-1.83 (m, 1H), 1.78-1.63 (m, 1H), 1.47-0.93 (m, 6H), one proton merged with solvent peaks; LCMS (Method B): Rt=0.99 min, m/z=656.5 [M+H]⁺; HPLC (Method A): Rt=4.64 min, 99.55%; Chiral SFC (Method H): Rt=5.03 min, 100%.

### Example 96. (D2)-5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(3-(1-methylazetidin-3-yl)cyclobutyl)benzamide (Compound No. 96)

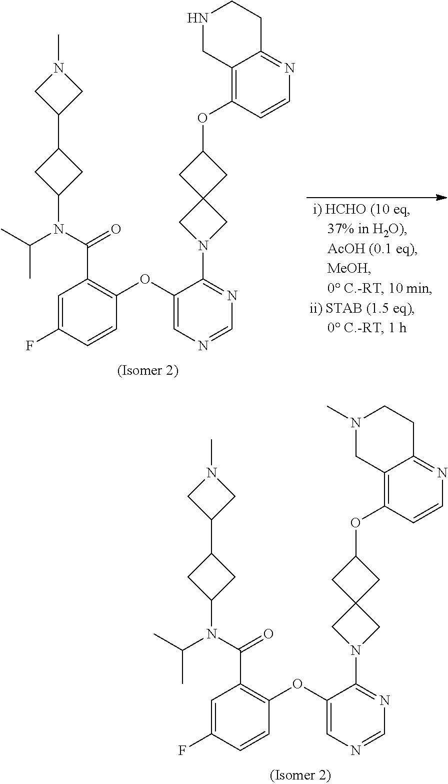 [SMILES: CC(C)N(C(=O)c1cc(F)ccc1Oc1cncnc1N1CC2(CC(Oc3ccnc4c3CNCC4)C2)C1)C1CC(C2CN(C)C2)C1 ; CC(C)N(C(=O)c1cc(F)ccc1Oc1cncnc1N1CC2(CC(Oc3ccnc4c3CN(C)CC4)C2)C1)C1CC(C2CN(C)C2)C1]

This compound was synthesized following the procedure described for the synthesis of Example 76, starting with 1 equivalent of (D2)-5-fluoro-N-isopropyl-N-(3-(1-methylazetidin-3-yl)cyclobutyl)-2-((4-(6-((5,6,7,8-tetrahydro-1,6-naphthyridin-4-yl)oxy)-2-azaspiro[3.3]heptan-2-yl)pyrimidin-5-yl)oxy)benzamide, except that 10 equivalents of formaldehyde (37% H₂O), 0.1 equivalent of AcOH, and 1.5 equivalents of STAB were used. After completion, the reaction was concentrated under reduced pressure. The crude product was purified by reverse phase chromatography using a SiliCycle SiliaSep C18 column (40 g) on Biotage isolera one (Mobile phase A: 10 mM NH₄HCO₃ in water, B: ACN) to afford (D2)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(3-(1-methylazetidin-3-yl)cyclobutyl)benzamide as a solid.

Yield: 48.8%; ¹H NMR (400 MHz, DMSO-d₆): δ 8.30-8.23 (m, 1H), 8.18 (d, J=5.5 Hz, 1H), 7.76-7.62 (m, 1H), 7.33-7.03 (m, 3H), 6.66 (d, J=5.6 Hz, 1H), 4.75 (quin, J=6.5 Hz, 1H), 4.37-3.90 (m, 6H), 3.86-3.61 (m, 1H), 3.37 (s, 2H), 3.20-2.90 (m, 3H), 2.85-2.75 (m, 5H), 2.73-2.67 (m, 2H), 2.63 (br t, J=5.9 Hz, 2H), 2.37 (s, 3H), 2.30-2.21 (m, 4H), 2.20-2.09 (m, 3H), 1.85-1.50 (br s, 1H), 1.48-0.90 (m, 6H); LCMS (Method B): Rt=1.0 min, m/z=656.3 [M+H]⁺; HPLC (Method A): Rt=4.51 min, 99.69%; Chiral SFC (Method L): Rt=1.97 min, 100%.

### Example 97. 2-((4-(6-((6-(3-(5-Oxa-2-azaspiro[3.4]octan-2-yl)propyl)-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-methylpiperidin-4-yl)benzamide (Compound No. 97)

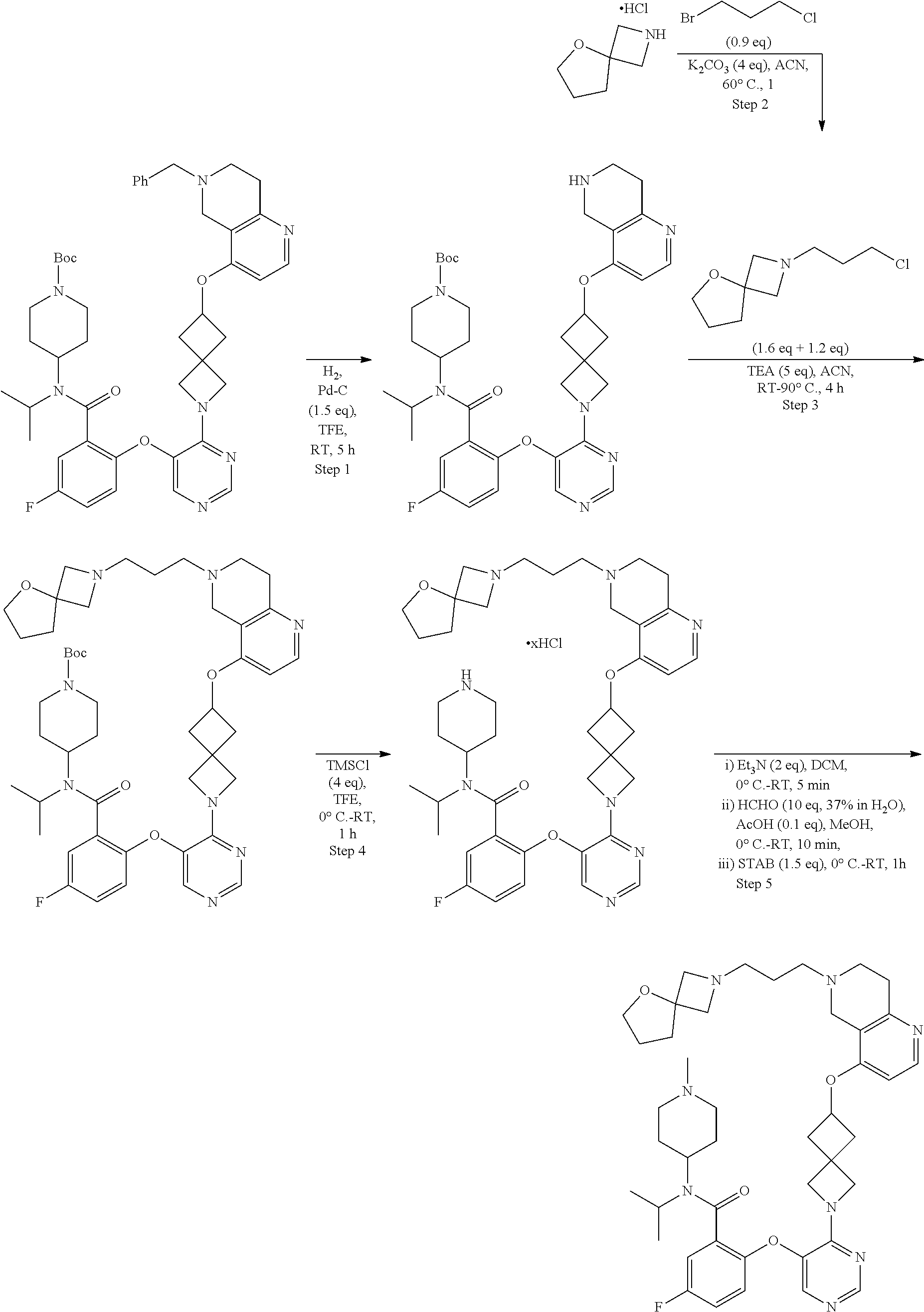

### Step 1. tert-Butyl 4-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)piperidine-1-carboxylate

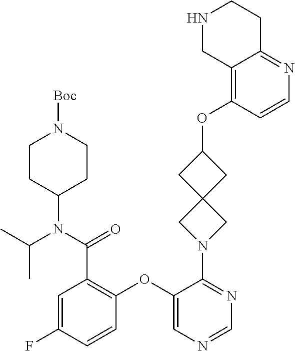 [SMILES: CC(C)N(C(=O)c1cc(F)ccc1Oc1cncnc1N1CC2(CC(Oc3ccnc4c3CNCC4)C2)C1)C1CCN(C(=O)OC(C)(C)C)CC1]

This compound was synthesized following the procedure described for the synthesis of Example 20, Step 5, starting with 1 equivalent of tert-butyl 4-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)piperidine-1-carboxylate, except that 1.5 equivalents of Pd-C was used. After completion, the reaction was filtered through a Celite® pad, and the pad was washed with MeOH. The filtrate was concentrated under reduced pressure. The resulting residue was diluted with aqueous NaHCO₃ solution and the mixture was extracted with 10% MeOH in DCM. The combined organic layer was concentrated under reduced pressure to obtain crude tert-butyl 4-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)piperidine-1-carboxylate as a solid. This material was used without further purification.

Yield: 75%; ¹H NMR (400 MHz, DMSO-d₆): δ 8.31-8.24 (m, 1H), 8.22-8.11 (m, 1H), 7.78-7.67 (m, 1H), 7.31-7.21 (m, 2H), 7.17-7.04 (m, 1H), 6.73-6.62 (m, 1H), 4.89-4.65 (m, 1H), 4.45-3.79 (m, H), 3.76-3.65 (m, 2H), 3.59-3.50 (m, 1H), 2.96 (br t, J=5.7 Hz, 2H), 2.89-2.75 (m, 5H), 2.72-2.65 (m, 2H), 2.31-2.19 (m, 2H), 1.72-1.48 (m, 2H), 1.44-1.27 (m, 11H), 1.15-0.80 (m, 6H).LCMS (Method B): Rt=1.29 min, m/z=702.3 [M+H]⁺, 93.11%.

### Step 2. 2-(3-Chloropropyl)-5-oxa-2-azaspiro[3.4]octane

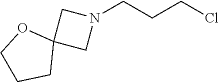 [SMILES: ClCCCN1CC2(CCCO2)C1]

In a 50 mL two-neck round bottom flask under a nitrogen atmosphere, 5-oxa-2-azaspiro[3.4]octane hydrochloride (500 mg, 3.34 mmol) was dissolved in ACN (10 mL). To this solution, K₂CO₃ (1847 mg, 13.37 mmol) and 1-bromo-3-chloropropane (474 mg, 3.01 mmol) were added at RT, and the reaction was stirred at 60° C. for 1 h. The reaction progress was monitored by TLC (10% MeOH in DCM). After completion, the reaction was quenched with ice-cold water (20 mL), and the mixture was extracted with EtOAc (2×70 mL). The combined organic layer was dried over anhydrous sodium sulfate and filtered, and the filtrate was concentrated under reduced pressure to afford crude 2-(3-chloropropyl)-5-oxa-2-azaspiro[3.4]octane (500 mg, 79% yield) as a liquid. This material was used without further purification. ¹H NMR (400 MHz, DMSO-d,): 5 3.69-3.59 (m, 4H), 3.27-3.23 (m, 2H), 2.93-2.88 (m, 2H), 2.47 (t, J=6.8 Hz, 2H), 2.02-1.94 (m, 2H), 1.85-1.75 (m, 2H), 1.71 (quin, J=6.7 Hz, 2H).

### Step 2. tert-Butyl 4-(2-((4-(6-((6-(3-(5-oxa-2-azaspiro[3.4]octan-2-yl)propyl)-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)piperidine-1-carboxylate

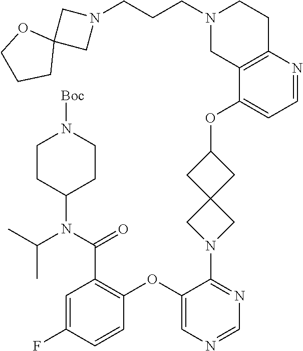 [SMILES: CC(C)N(C(=O)c1cc(F)ccc1Oc1cncnc1N1CC2(CC(Oc3ccnc4c3CN(CCCN3CC5(CCCO5)C3)CC4)C2)C1)C1CCN(C(=O)OC(C)(C)C)CC1]

In a 4 mL vial, tert-butyl 4-(5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamido)piperidine-1-carboxylate (500 mg, 0.712 mmol) and 2-(3-chloropropyl)-5-oxa-2-azaspiro[3.4]octane (216 mg, 1.140 mmol) were dissolved in ACN (5 mL). To this reaction mixture, Et₃N (0.501 mL, 3.56 mmol) was added, and the reaction was stirred at 90° C. for 4 h. The starting material was not consumed. Therefore, the reaction was cooled to RT and another lot of 2-(3-chloropropyl)-5-oxa-2-azaspiro[3.4]octane (162 mg, 0.855 mmol) was added. The reaction was stirred at 90° C. for 4 h. The reaction progress was monitored by LCMS. After completion, the reaction was concentrated under reduced pressure. The crude product was purified by reverse phase chromatography using a SiliCycle SiliaSep C18 column (120 g) on Biotage isolera one (Mobile phase A: 10 mM NH₄HCO₃ in water, B: ACN), then by achiral SFC (Method B) to obtain tert-butyl 4-(2-((4-(6-((6-(3-(5-oxa-2-azaspiro[3.4]octan-2-yl)propyl)-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)piperidine-1-carboxylate (120 mg, 14.64% yield) as a solid. LCMS (Method A): Rt=2.04 min, m/z=855.3 [M+H]⁺, 74.30%.

### Step 3. 2-((4-(6-((6-(3-(5-Oxa-2-azaspiro[3.4]octan-2-yl)propyl)-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(piperidin-4-yl)benzamide xHCl

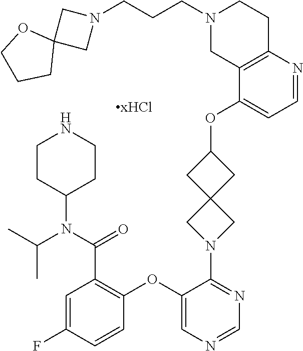 [SMILES: CC(C)N(C(=O)c1cc(F)ccc1Oc1cncnc1N1CC2(CC(Oc3ccnc4c3CN(CCCN3CC5(CCCO5)C3)CC4)C2)C1)C1CCNCC1]

This compound was synthesized following the procedure described for the synthesis of Example 20, Step 3, starting with 1 equivalent of tert-butyl 4-(2-((4-(6-((6-(3-(5-oxa-2-azaspiro[3.4]octan-2-yl)propyl)-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)piperidine-1-carboxylate. After completion, the reaction mixture was concentrated under reduced pressure. The residue was triturated with EtOAc, and the supernatant was decanted. The solid was dried under reduced pressure to obtain crude 2-((4-(6-((6-(3-(5-oxa-2-azaspiro[3.4]octan-2-yl)propyl)-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(piperidin-4-yl)benzamide xHCl as a solid. This material was used without further purification.

Yield: 78%; LCMS (Method B): Rt=0.90 min, m/z=755.4 [M+H]⁺, 74.95%.

### Step 4. 2-((4-(6-((6-(3-(5-Oxa-2-azaspiro[3.4]octan-2-yl)propyl)-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-methylpiperidin-4-yl)benzamide

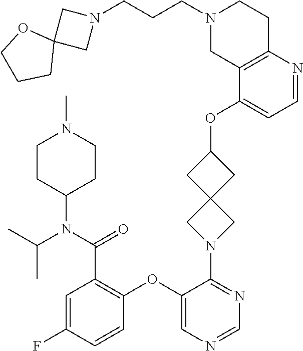 [SMILES: CC(C)N(C(=O)c1cc(F)ccc1Oc1cncnc1N1CC2(CC(Oc3ccnc4c3CN(CCCN3CC5(CCCO5)C3)CC4)C2)C1)C1CCN(C)CC1]

This compound was synthesized following the procedure described for the synthesis of Example 20, Step 4, starting with 1 equivalent of 2-((4-(6-((6-(3-(5-oxa-2-azaspiro[3.4]octan-2-yl)propyl)-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(piperidin-4-yl)benzamide hydrochloride, except that 0.1 equivalent of AcOH, and 1.5 equivalents of STAB were used. After completion, the reaction was concentrated under reduced pressure. The crude was purified by Prep HPLC (Method A) to obtain 2-((4-(6-((6-(3-(5-oxa-2-azaspiro[3.4]octan-2-yl)propyl)-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-methylpiperidin-4-yl)benzamide as a solid.

Yield: 34.0%; ¹H NMR (400 MHz, DMSO-d₆): δ 8.30-8.25 (m, 1H), 8.18 (d, J=5.6 Hz, 1H), 7.76-7.61 (m, 1H), 7.31-7.20 (m, 2H), 7.16-7.03 (m, 1H), 6.66 (d, J=5.6 Hz, 1H), 4.74 (quin, J=6.5 Hz, 1H), 4.38-4.17 (m, 3H), 4.16-4.08 (m, 1H), 3.77-3.68 (m, 11H), 3.65 (t, J==6.8 Hz, 2H), 3.57-3.48 (m, 1H), 3.39 (s, 2H), 3.27-3.21 (m, 2H), 3.15-2.96 (m, 1H), 2.91-2.84 (m, 2H), 2.83-2.72 (m, 6H), 2.69-2.62 (m, 3H), 2.48-2.44 (m, 2H), 2.39 (br t, J=7.1 Hz, 2H), 2.30-2.21 (m, 2H), 2.17-2.03 (m, 3H), 2.01-1.94 (m, 2H), 1.94-1.85 (m, 1H), 1.80 (quin, J=7.0 Hz, 2H), 1.68-1.58 (m, 2H), 1.55-1.46 (m, 2H), 1.45-0.92 (m, 6H), one proton merged with solvent peaks: LCMS (Method C: Rt=1.38 min, m/z=769.4 [M+H]⁺; HPLC (Method A): Rt=4.67 min, 99.89%.

### Example 98. 5-Fluoro-N-isopropyl-N-((1r,3r)-3-morpholinocyclobutyl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 98)

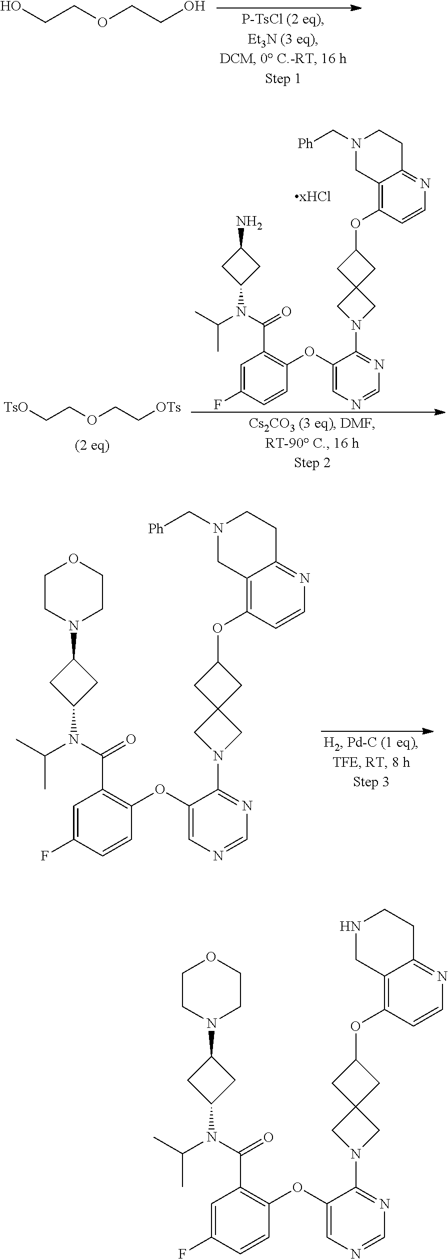 [SMILES: OCCOCCO ; CC(C)N(C(=O)c1cc(F)ccc1Oc1cncnc1N1CC2(CC(Oc3ccnc4c3CN(Cc3ccccc3)CC4)C2)C1)[C@H]1C[C@H](N)C1 ; Cc1ccc(S(=O)(=O)OCCOCCOS(=O)(=O)c2ccc(C)cc2)cc1 ; CC(C)N(C(=O)c1cc(F)ccc1Oc1cncnc1N1CC2(CC(Oc3ccnc4c3CN(Cc3ccccc3)CC4)C2)C1)[C@H]1C[C@H](N2CCOCC2)C1 ; CC(C)N(C(=O)c1cc(F)ccc1Oc1cncnc1N1CC2(CC(Oc3ccnc4c3CNCC4)C2)C1)[C@H]1C[C@H](N2CCOCC2)C1]

### Step 1. Oxybis(ethane-2,1-diyl)bis(4-methylbenzenesulfonate)

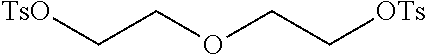 [SMILES: Cc1ccc(S(=O)(=O)OCCOCCOS(=O)(=O)c2ccc(C)cc2)cc1]

To a stirred solution of 2,2′-oxybis(ethan-9-66(0.6 g, 18.85 mmol)) in DCM (20 mL), TEA (7.88 mL, 56.5 mmol) was added at 0° C. To this reaction mixture, p-TsCl (7.19 g, 37.7 mmol) was added, and the reaction was stirred at RT for 16 h. The reaction progress was monitored by TLC (30% EtOAc in hexane). After completion, the reaction was cooled to 0° C. and quenched with saturated NaHCO₃ solution (25 mL), and the mixture was extracted with DCM (3×40 mL). The combined organic layer was washed with brine (25 mL), dried over sodium sulfate, and filtered. The filtrate was concentrated under reduced pressure. The crude was purified by silica gel flash column chromatography using EtOAc in hexane (product eluted at 10% EtOAc in hexane) to obtain oxybis(ethane-2,1-diyl)bis(4-methylbenzenesulfonate) (3.5 g, 44.8% yield) as a solid. 1H NMR (400 MHz, DMSO-d₆): 6.78 (d, J=8.3 Hz, 4H), 7.48 (d, J=8.0 Hz, 4H), 4.08-4.03 (m, 4H), 3.54-3.49 (m, 4H), 2.42 (s, 6H).

### Step 2. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-morpholinocyclobutyl)benzamide

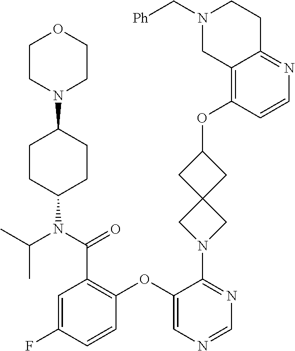 [SMILES: CC(C)N(C(=O)c1cc(F)ccc1Oc1cncnc1N1CC2(CC(Oc3ccnc4c3CN(Cc3ccccc3)CC4)C2)C1)[C@H]1CC[C@H](N2CCOCC2)CC1]

To a stirred solution of N-((1r,3r)-3-aminocyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl in DMF (5 mL), cesium carbonate (0.411 g, 1.260 mmol) was added at RT. To this reaction mixture, oxybis(ethane-2,1-diyl)bis(4-methylbenzenesulfonate) (0.348 g, 0.840 mmol) was added and the reaction was stirred at 90° C. for 16 h. The reaction progress was monitored by LCMS. After completion, the reaction was cooled to RT, quenched with ice-cold water (10 mL), and extracted with EtOAc (3×25 mL). The combined organic layer was washed with brine (30 mL), dried over sodium sulfate, and filtered. The filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-morpholinocyclobutyl)benzamide (0.085 g, 19.07% yield). LCMS (Method A): Rt=1.99 min, m/z=748.2 [M+H]⁺, 85.25%.

### Step 3. 5-Fluoro-N-isopropyl-N-((1r,3r)-3-morpholinocyclobutyl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

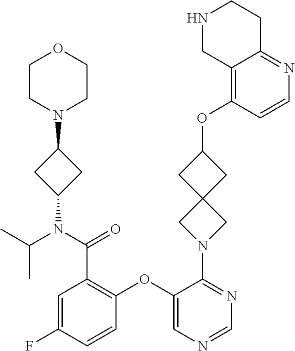 [SMILES: CC(C)N(C(=O)c1cc(F)ccc1Oc1cncnc1N1CC2(CC(Oc3ccnc4c3CNCC4)C2)C1)[C@H]1C[C@H](N2CCOCC2)C1]

This compound was synthesized following the procedure described for the synthesis of Example 20, step 5, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-morpholinocyclobutyl)benzamide, except that 1 equivalent of Pd-C was used. After filtration and concentration, the crude was purified by reverse phase chromatography using a SiliCycle SiliaSep C18 column (40 g) on Biotage isolera one (Mobile phase A: 10 mM NH₄HCO₃ in water, B: ACN) to obtain 5-fluoro-N-isopropyl-N-((1r,3r)-3-morpholinocyclobutyl)-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 36.4%, ¹H NMR (400 MHz, DMSO-d₆): δ 8.29-8.26 (m, 1H), 8.16 (d, J=5.5 Hz, 1H), 7.76-7.67 (m, 1H), 7.32-7.22 (m, 2H), 7.17-7.06 (m, 1H), 6.64 (d, J=5.6 Hz, 1H), 4.77-4.69 (m, 1H), 4.34-4.07 (m, 4H), 4.03-3.92 (m, 1H), 3.81-3.64 (m, 3H), 3.62-3.46 (m, 4H), 3.00-2.87 (m, 4H), 2.82-2.70 (m, 3H), 2.72-2.68 (m, 2H), 2.30-1.99 (m, 9H), 1.49-0.88 (m, 6H; LCMS (Method A): Rt=1.49 min, m/z=658.3 [M+H]⁺; HPLC (Method A): Rt=4.28 min, 99.50%; Chiral SFC (Method C): Rt=4.94 min, 100%.

### Example 99. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((1r,3r)-3-morpholinocyclobutyl)benzamide (Compound No. 99)

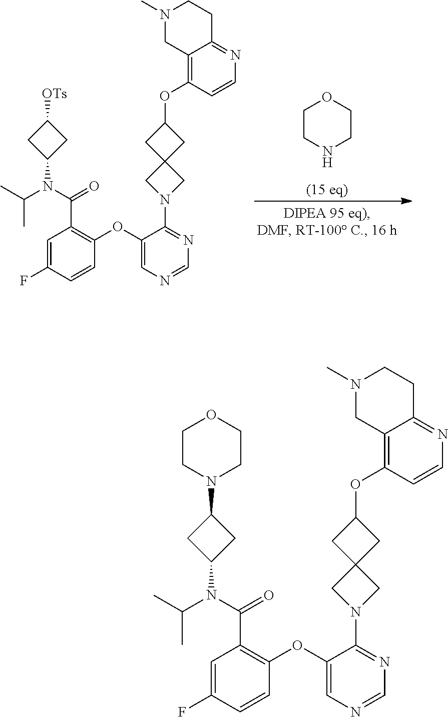 [SMILES: Cc1ccc(S(=O)(=O)O[C@H]2C[C@@H](N(C(=O)c3cc(F)ccc3Oc3cncnc3N3CC4(CC(Oc5ccnc6c5CN(C)CC6)C4)C3)C(C)C)C2)cc1 ; C1COCCN1 ; CC(C)N(C(=O)c1cc(F)ccc1Oc1cncnc1N1CC2(CC(Oc3ccnc4c3CN(C)CC4)C2)C1)[C@H]1C[C@H](N2CCOCC2)C1]

In a 20 mL pressure vial, (1s,3s)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclobutyl 4-methylbenzenesulfonate (190 mg, 0.251 mmol) was dissolved in DMF (3 mL). To this solution, DIPEA (162 mg, 1.255 mmol) and morpholine (0.324 mL, 3.77 mmol) were added at RT. The reaction was stirred at 100° C. for 16 h. The reaction progress was monitored by LCMS. After completion, the reaction was concentrated under reduced pressure. The crude was purified by Prep HPLC (Method A) to obtain 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((1r,3r)-3-morpholinocyclobutyl)benzamide (30 mg, 17.76% yield) as a solid. ¹H NMR (400 MHz, DMSO-d₆): δ 8.29-8.25 (m, 1H), 8.18 (d, J=5.6 Hz, 1H), 7.76-7.67 (m, 1H), 7.32-7.22 (m, 2H), 7.16-7.06 (m, 1H), 6.65 (d, J=5.6 Hz, 1H), 4.75 (quin, J=6.3 Hz, 1H), 4.34-4.07 (m, 4H), 4.03-3.92 (m, 1H), 3.80-3.64 (m, 1H), 3.61-3.48 (m, 4H), 3.37 (br s, 2H), 3.00-2.86 (m, 1H), 2.85-2.71 (m, 5H), 2.65-2.61 (m, 2H), 2.37 (s, 3H), 2.28-2.201 (m, 4H), 2.19-2.03 (m, 4H), 1.50-0.88 (m, 6H), one proton merged with solvent peaks; LCMS (Method A): Rt=1.62 min, m/z=672.2 [M+H]⁺; HPLC (Method A): Rt=4.71 min, 99.82%; Chiral SFC (Method T): Rt=3.21 min, 100%.

### Example 100. N-((1r,3r)-3-(Azetidin-1-yl)cyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 100)

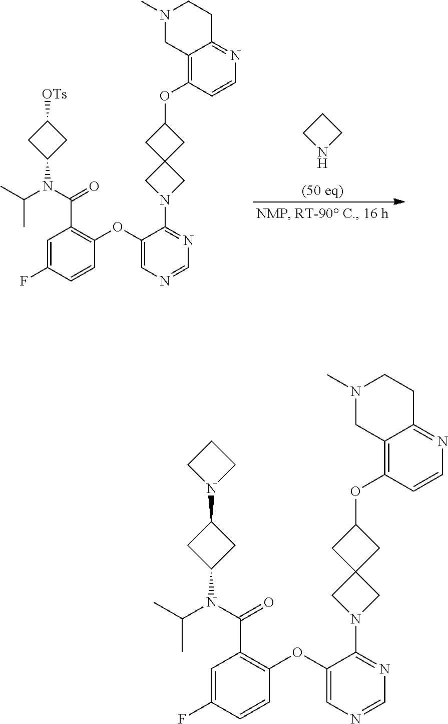

In a 20 mL pressure vial, (1s,3s)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclobutyl 4-methylbenzenesulfonate (150 mg, 0.198 mmol) was dissolved in NMP (2 mL). To this solution, azetidine (566 mg, 9.91 mmol) was added at RT and the reaction was stirred at 90° C. for 16 h. The reaction progress was monitored by LCMS. After completion, the reaction was diluted with cold water (30 mL), and the mixture was extracted with EtOAc (3×30 mL). The combined organic layer was washed with brine solution (30 mL), dried over sodium sulfate, and filtered, and the filtrate was concentrated under reduced pressure. The crude compound was purified by reverse phase chromatography using a SiliCycle SiliaSep C18 column (40 g) on Biotage isolera one (Mobile phase A: 10 mM NH₄HCO₃ in water, B: ACN) to obtain N-((1r,3r)-3-(azetidin-1-yl)cyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (30 mg, 23.03% yield) as a solid. ¹H NMR (400 MHz, DMSO-d₆): δ 8.30-8.26 (m, 1H), 8.18 (d, J=5.6 Hz, 1H), 7.76-7.65 (m, 1H), 7.33-7.21 (m, 2H), 7.17-7.02 (m, 1H), 6.66 (d, J=5.8 Hz, 1H), 4.79-4.70 (m, 1H), 4.34-3.94 (m, 5H), 3.79-3.63 (m, 1H), 3.40-3.36 (m, 2H), 3.05-2.88 (m, 5H), 2.86-2.74 (m, 5H), 2.63 (t, J=5.5 Hz, 2H), 2.37 (s, 3H), 2.31-2.19 (m, 3H), 2.14-2.01 (m, 1H), 1.95-1.64 (m, 3H), 1.47-1.24 (m, 3H), 1.09-0.90 (m, 3H); LCMS (Method A): Rt=1.77 min, m/z=642.1 [M+H]⁺; HPLC (Method C): Rt=8.14 min, 97.63%; Chiral SFC (Method AJ): Rt=3.17 min, 100%.

### Example 101. N-((1r,3r)-3-(5-Oxa-2-azaspiro[3.4]octan-2-yl)cyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 101)

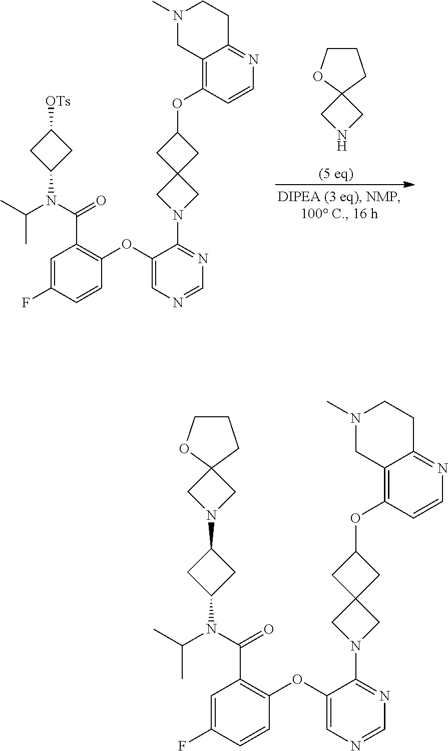 [SMILES: Cc1ccc(S(=O)(=O)O[C@H]2C[C@@H](N(C(=O)c3cc(F)ccc3Oc3cncnc3N3CC4(CC(Oc5ccnc6c5CN(C)CC6)C4)C3)C(C)C)C2)cc1 ; C1COC2(C1)CNC2 ; CC(C)N(C(=O)c1cc(F)ccc1Oc1cncnc1N1CC2(CC(Oc3ccnc4c3CN(C)CC4)C2)C1)[C@H]1C[C@H](N2CC3(CCCO3)C2)C1]

In a 20 mL pressure vial, (1s,3s)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclobutyl 4-methylbenzenesulfonate (300 mg, 0.396 mmol) and 5-oxa-2-azaspiro[3.4]octane (224 mg, 1.982 mmol) were dissolved in NMP (5 mL). To this mixture, DIPEA (0.208 mL, 1.189 mmol) was added and the reaction was stirred at 100° C. for 16 h. The reaction progress was monitored by TLC (10% MeOH in DCM). After completion, the reaction was concentrated under reduced pressure. The crude was purified by Prep HPLC (Method E) to obtain N-((1r,3r)-3-(5-oxa-2-azaspiro[3.4]octan-2-yl)cyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (56 mg, 20.14% yield) as a solid. ¹H NMR (400 MHz, DMSO-d₆): δ 8.28 (s, 1H), 8.18 (d, J=5.6 Hz, 1H), 7.77-7.67 (m, 1H), 7.34-7.02 (m, 3H), 6.66 (d, J=5.6 Hz, 1H), 4.82-4.68 (m, 1H), 4.36-4.08 (m, 4H), 4.06-3.95 (m, 1H), 3.82-3.56 (m, 4H), 3.39-3.36 (m, 2H), 3.24-3.18 (m, 1H), 3.15-2.96 (m, 2H), 2.91-2.73 (m, 6H), 2.63 (br t, J=5.8 Hz, 2H), 2.37 (s, 3H), 2.30-2.21 (m, 2H), 2.18-2.06 (m, 1H), 2.02-1.96 (m, 1H), 1.94-1.87 (m, 1H), 1.86-1.67 (m, 4H), 1.48-0.86 (m, 6H); LCMS (Method C): Rt=1.44 min, m/z=698.3 [M+H]⁺; HPLC (Method C): Rt=8.73 min, 99.49%; Chiral SFC (Method E): Rt=3.10 min, 97.76%.

### Example 102. 5-Fluoro-N-isopropyl-N-(1-isopropylazetidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 102)

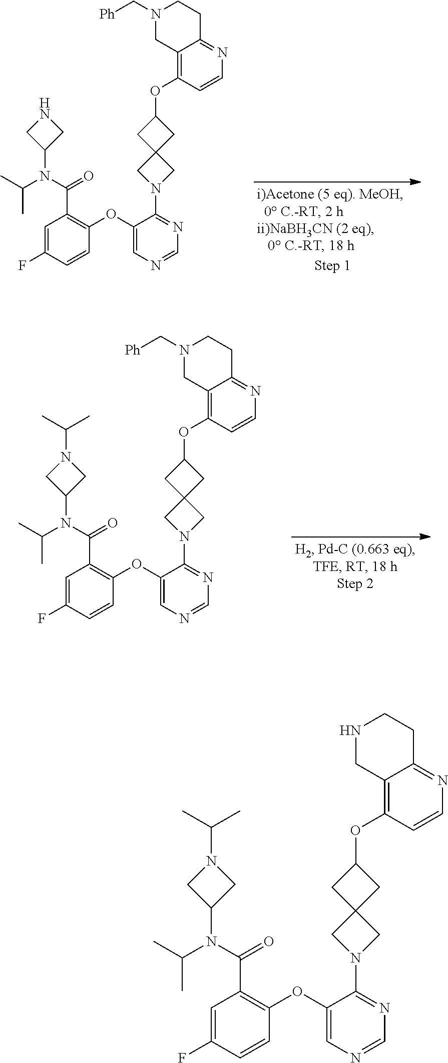 [SMILES: CC(C)N(C(=O)c1cc(F)ccc1Oc1cncnc1N1CC2(CC(Oc3ccnc4c3CN(Cc3ccccc3)CC4)C2)C1)C1CNC1 ; CC(C)N1CC(N(C(=O)c2cc(F)ccc2Oc2cncnc2N2CC3(CC(Oc4ccnc5c4CN(Cc4ccccc4)CC5)C3)C2)C(C)C)C1 ; CC(C)N1CC(N(C(=O)c2cc(F)ccc2Oc2cncnc2N2CC3(CC(Oc4ccnc5c4CNCC5)C3)C2)C(C)C)C1]

### Step 1. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-isopropylazetidin-3-y)benzamide

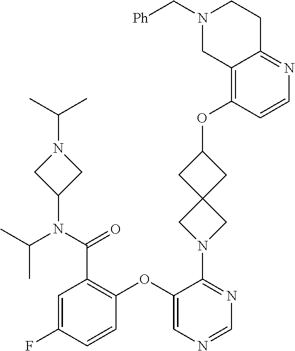 [SMILES: CC(C)N1CC(N(C(=O)c2cc(F)ccc2Oc2cncnc2N2CC3(CC(Oc4ccnc5c4CN(Cc4ccccc4)CC5)C3)C2)C(C)C)C1]

To a stirred solution of N-(azetidin-3-yl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (350 mg, 0.527 mmol) in MeOH (10 mL), acetone (0.194 mL, 2.64 mmol) was added at 0° C. The reaction was stirred at RT for 2 h. To this reaction mixture, sodium cyanoborohydride (66.3 mg, 1.055 mmol) was added at 0° C. The reaction was stirred at RT for 18 h, and was monitored by TLC (10% MeOH in DCM). The reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-isopropylazetidin-3-yl)benzamide (250 mg, 59.3% yield) as a sticky solid. LCMS (Method B): Rt=1.19 min, m/z=706.3 [M+H]⁺, 88.34%.

### Step 2. S-Fluoro-N-isopropyl-N-(1-isopropylazetidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

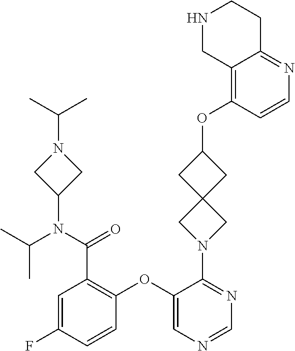 [SMILES: CC(C)N1CC(N(C(=O)c2cc(F)ccc2Oc2cncnc2N2CC3(CC(Oc4ccnc5c4CNCC5)C3)C2)C(C)C)C1]

This compound was synthesized following the procedure described for the synthesis of Example 5, Step 1, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(1-isopropylazetidin-3-yl)benzamide, except that 0.663 equivalent of Pd-C was used. After filtration and concentration, the crude was purified by reverse phase chromatography using a SiliCycle SiliaSep C18 column (40 g) on Biotage isolera one (Mobile phase A: 10 mM NH₄HCO₃ in water, B: ACN) to obtain 5-fluoro-N-isopropyl-N-(1-isopropylazetidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 20.72%; ¹H NMR (400 MHz, DMSO-d₆): δ 8.33-8.22 (m, 1H), 8.16 (d, J=5.6 Hz, 1H), 7.79-7.69 (m, 1H), 7.36-7.02 (m, 3H), 6.63 (br d, J=5.5 Hz, 1H), 4.78-4.68 (m, 1H), 4.36-3.92 (m, 6H), 3.75-3.58 (m, 3H), 3.57-3.39 (m, 2H), 3.27-3.19 (m, 1H), 3.17-2.99 (m, 1H), 2.98-2.89 (m, 2H), 2.85-2.73 (m, 3H), 2.71-2.62 (m, 2H), 2.30-2.19 (m, 3H), 1.49-1.26 (m, 3H), 1.13-0.97 (m, 3H), 0.89-0.72 (m, 6H), one proton merged with solvent peaks; LCMS (Method H): Rt=0.95 min, m/z=616.4 [M+H]⁺, 96.14%; HPLC (Method A): Rt=4.22 min, 83.13%.

### Example 103. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(pyrrolidin-1-yl)cyclobutyl)benzamide (Compound No. 103)

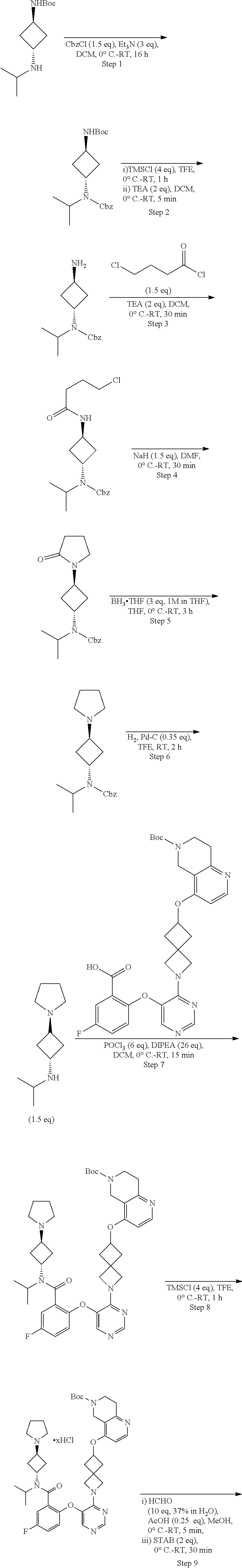

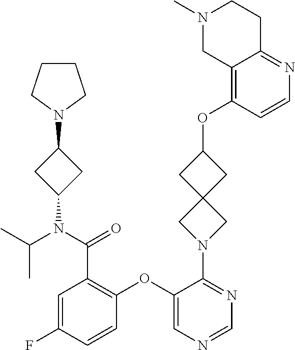 [SMILES: CC(C)N(C(=O)c1cc(F)ccc1Oc1cncnc1N1CC2(CC(Oc3ccnc4c3CN(C)CC4)C2)C1)[C@H]1C[C@H](N2CCCC2)C1]

### Step 1. Benzyl ((1r,3r)-3-((tert-butoxycarbonyl)amino)cyclobutyl)(isopropyl)carbamate

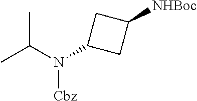

In a 100 mL round bottom flask under a nitrogen atmosphere, tert-butyl ((1r,3r)-3-(isopropylamino)cyclobutyl)carbamate (1.1 g, 4.82 mmol) was dissolved in DCM (12 mL). To this solution, Et₃N (1.919 mL, 14.45 mmol) and Cbz-C1 (1.032 mL, 7.23 mmol) were added at 0° C., and the reaction was stirred at RT for 16 h. The reaction progress was monitored by TLC (20% EtOAc in hexane). After completion, the reaction was quenched with water (20 mL) and the mixture was extracted with DCM (3×50 mL). The combined organic layer was washed with brine solution (30 mL), dried over anhydrous sodium sulfate, and filtered. The filtrate was concentrated under reduced pressure. The crude was purified by silica gel flash column chromatography using EtOAc in hexane (product eluted at 10% EtOAc in hexane) to obtain benzyl ((1r,3r)-3-((tert-butoxycarbonyl)amino)cyclobutyl)(isopropyl)carbamate (0.500 g, 28.6% yield) as a solid. ¹HNMR (400 MHz, DMSO-d₆): δ 7.42-7.32 (m, 6H), 5.09 (s, 2H), 4.41-4.31 (m, 1H), 4.12-3.99 (m, 1H), 3.86-3.75 (m, 1H), 2.66-2.60 (m, 2H), 2.05-1.94 (m, 2H), 1.37 (s, 9H), 1.10 (d, J=6.8 Hz, 6H).

### Step 2. Benzyl ((1r,3r)-3-aminocyclobutyl)(isopropyl)carbamate

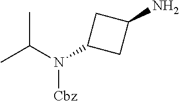

To a stirred solution of benzyl ((1r,3r)-3-((tert-butoxycarbonyl)amino)cyclobutyl)(isopropyl)carbamate (500 mg, 1.379 mmol) in TFE (5 mL), TMSCl (0.705 mL, 5.52 mmol) was added at 0° C., and the reaction was stirred at RT for 1 h. The reaction progress was monitored by TLC (10% MeOH in DCM). After completion, the reaction was concentrated under reduced pressure. The crude was dissolved in DCM, Et₃N (0.385 mL, 2.76 mmol) was added at 0° C., and the mixture was stirred at RT for 5 min. The mixture was concentrated under reduced pressure to obtain crude benzyl ((1r,3r)-3-aminocyclobutyl)(isopropyl)carbamate (440 mg, quantitative yield) as a semi-solid. This material was used without further purification. LCMS (Method A): Rt=1.58 min, m/z=263.1 [M+H]⁺, 97.79%.

### Step 3. Benzyl ((1r,3r)-3-(4-chlorobutanamido)cyclobutyl)(isopropyl)carbamate

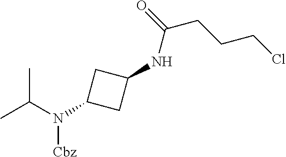 [SMILES: CC(C)N(C(=O)OCc1ccccc1)[C@H]1C[C@H](NC(=O)CCCCl)C1]

In a dried, 25 mL round bottom flask under a nitrogen atmosphere, benzyl ((1r,3r)-3-aminocyclobutyl)(isopropyl)carbamate (440 mg, 1.677 mmol) was dissolved in DCM (8 mL). To this solution, Et₃N (0.584 mL, 4.19 mmol) and 4-chlorobutanoyl chloride (0.282 mL, 2.52 mmol) were added at 0° C., and the reaction was stirred at RT for 1 h. The reaction progress was monitored by TLC (10% MeOH in DCM). After completion, the reaction was quenched with water (10 mL) and the mixture was extracted with DCM (3×20 mL). The combined organic layer was washed with brine solution (10 mL), dried over anhydrous sodium sulfate, and filtered. The filtrate was concentrated under reduced pressure. The crude was purified by silica gel flash column chromatography using MeOH in DCM (product eluted at 2% MeOH in DCM) to obtain benzyl ((1r,3r)-3-(4-chlorobutanamido)cyclobutyl)(isopropyl)carbamate (570 mg, 88% yield) as a gummy liquid. ¹H NMR (400 MHz, DMSO-d₆): δ 8.28 (d, J=6.8 Hz, 1H), 7.45-7.29 (m, 5H), 5.10 (s, 2H), 4.49-4.33 (m, 1H), 4.04-3.96 (m, 2H), 3.63 (t, J=6.5 Hz, 2H), 2.76-2.63 (m, 2H), 2.25-2.19 (m, 2H), 2.04-1.96 (m, 2H), 1.96-1.89 (m, 2H), 1.12 (d, J=6.9 Hz, 6H); LCMS (Method A): Rt=1.97 min, m/z=367.0 [M+H]⁺, 94.80%.

### Step 4. Benzyl Isopropyl((1r,3r)-3-(2-oxopyrrolidin-1-yl)cyclobutyl)carbamate

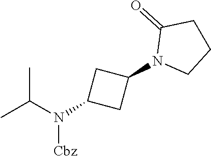 [SMILES: CC(C)N(C(=O)OCc1ccccc1)[C@H]1C[C@H](N2CCCC2=O)C1]

In a dried, 25 mL round bottom flask under a nitrogen atmosphere, benzyl ((1r,3r)-3-(4-chlorobutanamido)cyclobutyl)(isopropyl)carbamate (520 mg, 1.417 mmol) was dissolved in DMF (5 mL). To this solution, NaH (85 mg, 2.126 mmol) was added at 0° C. The reaction was stirred at 0° C. for 10 min then at RT for 30 min. The reaction progress was monitored by TLC (3% MeOH in DCM). After completion, the reaction was cooled to 0° C. and quenched with water (10 mL). The mixture was extracted with EtOAc (3×20 mL). The combined organic layer was washed with brine solution (15 mL), dried over anhydrous sodium sulfate, and filtered. The filtrate was concentrated under reduced pressure. The crude was purified by silica gel flash column chromatography using MeOH in DCM (product eluted at 2% MeOH in DCM) to obtain benzyl isopropyl((1r,3r)-3-(2-oxopyrrolidin-1-yl)cyclobutyl)carbamate (260 mg, 55.4% yield) as a solid. ¹H NMR (400 MHz, DMSO-d₆): δ 7.43-7.29 (m, 5H), 5.12 (s, 2H), 4.62-4.48 (m, 1H), 4.37-4.23 (m, 1H), 4.10 (quin, J=6.8 Hz, 1H), 3.53 (t, J=6.9 Hz, 2H), 2.71-2.61 (m, 2H), 2.43-2.32 (m, 2H), 2.25-2.17 (m, 2H), 2.01-1.88 (m, 2H), 1.11 (d, J=6.8 Hz, 6H); LCMS (Method A): Rt=1.85 min, m/z=331.1 [M+H]⁺, 99.70%.

### Step 5. Benzyl Isopropyl((1r,3r)-3-(pyrrolidin-1-yl)cyclobutyl)carbamate

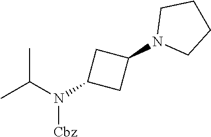 [SMILES: CC(C)N(C(=O)OCc1ccccc1)[C@H]1C[C@H](N2CCCC2)C1]

In a dried, 25 mL round bottom flask under a nitrogen atmosphere, benzyl isopropyl((1r,3r)-3-(2-oxopyrrolidin-1-yl)cyclobutyl)carbamate (210 mg, 0.636 mmol) was dissolved in THF (8 mL). To this solution, BH₃·THF (1.907 mL, 1.907 mmol, 1 M in THF) was added at 0° C. and the reaction was stirred at RT for 3 h. The reaction progress was monitored by TLC (100% EtOAC). After completion, the reaction mixture was concentrated under reduced pressure. The residue was dissolved in MeOH (6 mL) and the mixture was stirred at 60° C. for 24 h. The reaction was concentrated under reduced pressure to obtain crude benzyl isopropyl((1r,3r)-3-(pyrrolidin-1-yl)cyclobutyl)carbamate (140 mg, 57.4% yield) as a liquid. This material was used without further purification. ¹H NMR (400 MHz, DMSO-d₆): δ 7.43-7.30 (m, 5H), 5.08 (s, 2H), 4.29 (quin, J=8.6 Hz, 1H), 4.08-3.94 (m, 1H), 2.73-2.64 (m, 1H), 2.48-2.39 (m, 2H), 2.36-2.28 (m, 4H), 2.11-2.01 (m, 2H), 1.72-1.65 (m, 4H), 1.12 (d, J=6.7 Hz, 6H); LCMS (Method A): Rt=1.81 min, m/z=317.7 [M+H]⁺, 82.49%.

### Step 6. (1r,3r)—N-Isopropyl-3-(pyrrolidin-1-yl)cyclobutan-1-amine

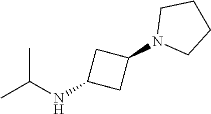

To a stirred solution of benzyl isopropyl((1r,3r)-3-(pyrrolidin-1-yl)cyclobutyl)carbamate (110 mg, 0.348 mmol) in TFE (3 mL), Pd—C (129 mg, 0.122 mmol) was added, and the reaction was stirred at RT for 2 h under a hydrogen atmosphere (balloon pressure). The reaction progress was monitored by TLC (10% MeOH in DCM). After completion, the reaction was filtered through a Celite® pad, and the filter pad was washed with TFE (50 mL). The filtrate was concentrated under reduced pressure to obtain crude (1r,3r)—N-isopropyl-3-(pyrrolidin-1-yl)cyclobutan-1-amine (60 mg, 95% yield) as a liquid. This material was used without further purification. ¹H NMR (400 MHz, DMSO-d₆): δ 2.80-2.72 (m, 1H), 2.71-2.63 (m, 1H), 2.39-2.30 (m, 4H), 2.12-2.01 (m, 2H), 1.78-1.62 (m, 6H), 0.92 (d, J=6.3 Hz, 6H), two protons merged with solvent peaks.

### Step 7. tert-Butyl 4-((2-(5-(4-fluoro-2-(isopropyl((1r,3r)-3-(pyrrolidin-1-yl)cyclobutyl)carbamoyl)phenoxyl)pyrimidin-4-yl)-2-azaspiro[3.3]heptan-6-yl)oxy)-7,8-dihydro-1,6-naphthyridine-6(5H)-carboxylate

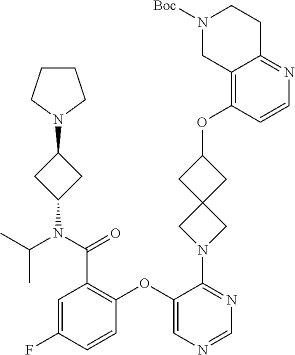 [SMILES: CC(C)N(C(=O)c1cc(F)ccc1Oc1cncnc1N1CC2(CC(Oc3ccnc4c3CN(C(=O)OC(C)(C)C)CC4)C2)C1)[C@H]1C[C@H](N2CCCC2)C1]

This compound was synthesized following the procedure described for the synthesis of Example 20, step 2, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid (125 mg, 0.216 mmol) and 1.5 equivalents of (1r,3r)—N-isopropyl-3-(pyrrolidin-1-yl)cyclobutan-1-amine. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl 4-((2-(5-(4-fluoro-2-(isopropyl((1r,3r)-3-(pyrrolidin-1-yl)cyclobutyl)carbamoyl)phenoxyl)pyrimidin-4-yl)-2-azaspiro[3.3]heptan-6-yl)oxy)-7,8-dihydro-1,6-naphthyridine-6(5H)-carboxylate as a gummy liquid.

Yield: 18.89%; LCMS (Method A): Rt=2.21 min, m/z=742.2 [M+H]⁺, 86.64%.

### Step 8. 5-Fluoro-N-isopropyl-N-((1r,3r)-3-(pyrrolidin-1-yl)cyclobutyl)-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl

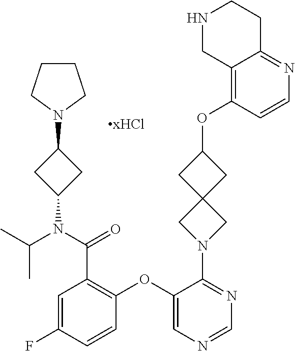 [SMILES: CC(C)N(C(=O)c1cc(F)ccc1Oc1cncnc1N1CC2(CC(Oc3ccnc4c3CNCC4)C2)C1)[C@H]1C[C@H](N2CCCC2)C1]

This compound was synthesized following the procedure described for the synthesis of Example 20, step 3, starting with 1 equivalent of tert-butyl 4-((2-(5-(4-fluoro-2-(isopropyl((1r,3r)-3-(pyrrolidin-1-yl)cyclobutyl)carbamoyl)phenoxyl)pyrimidin-4-yl)-2-azaspiro[3.3]heptan-6-yl)oxy)-7,8-dihydro-1,6-naphthyridine-6(5H)-carboxylate. After completion, the reaction was concentrated under reduced pressure and the residue was triturated with EtOAc. The organic layer was decanted and the solid was dried under reduced pressure to obtain crude 5-fluoro-N-isopropyl-N-((1r,3r)-3-(pyrrolidin-1-yl)cyclobutyl)-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl as a gummy liquid. This material was used without further purification. Quantitative yield; LCMS (Method C): Rt=1.41 min, m/z=642.3 [M+H]⁺, 94.40%.

### Step 9. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(pyrrolidin-1-yl)cyclobutyl)benzamide

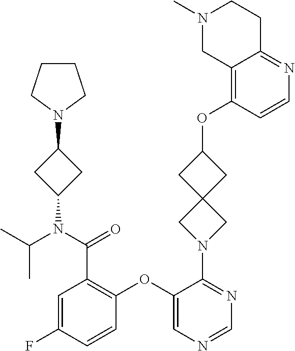 [SMILES: CC(C)N(C(=O)c1cc(F)ccc1Oc1cncnc1N1CC2(CC(Oc3ccnc4c3CN(C)CC4)C2)C1)[C@H]1C[C@H](N2CCCC2)C1]

This compound was synthesized following the procedure described for the synthesis of Example 76, starting with 1 equivalent of 5-fluoro-N-isopropyl-N-((1r,3r)-3-(pyrrolidin-1-yl)cyclobutyl)-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl, except that 10 equivalents of formaldehyde (37% in H₂O), 0.25 equivalent of AcOH, and 2 equivalents of STAB were used. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: MeOH) to obtain 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(pyrrolidin-1-yl)cyclobutyl)benzamide as a solid.

Yield: 27.2%; ¹H NMR (400 MHz, DMSO-d₆): δ 8.29-8.27 (m, 1H), 8.18 (d, J=5.6 Hz, 1H), 7.79-7.64 (m, 1H), 7.35-7.03 (m, 3H), 6.65 (br d, J=5.4 Hz, 1H), 4.81-4.69 (m, 1H), 4.36-3.97 (m, 5H), 3.82-3.60 (m, 1H), 3.37 (br s, 2H), 3.11-2.87 (m, 2H), 2.84-2.75 (m, 4H), 2.66-2.58 (m, 3H), 2.36 (s, 3H), 2.31-2.15 (m, 5H), 2.11-1.91 (m, 2H), 1.75-1.58 (m, 4H), 1.49-1.27 (m, 3H), 1.11-0.82 (m, 3H), one proton merged with solvent peaks; LCMS (Method B): Rt=0.95 min, m/z=656.2 [M+H]⁺; HPLC (Method A): Rt=4.78 min, 98.50%; Chiral SFC (Method AF): Rt=3.86 min, 100%.

### Example 104. N-((1r,3r)-3-(6-Oxa-2-azaspiro[3.4]octan-2-yl)cyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 104)

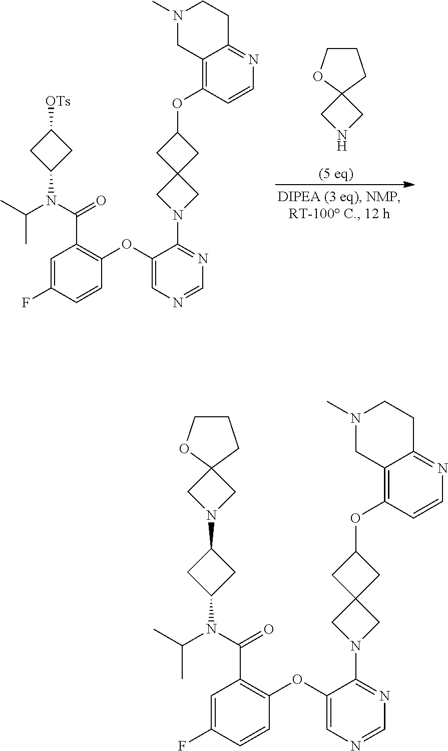 [SMILES: Cc1ccc(S(=O)(=O)O[C@H]2C[C@@H](N(C(=O)c3cc(F)ccc3Oc3cncnc3N3CC4(CC(Oc5ccnc6c5CN(C)CC6)C4)C3)C(C)C)C2)cc1 ; C1COC2(C1)CNC2 ; CC(C)N(C(=O)c1cc(F)ccc1Oc1cncnc1N1CC2(CC(Oc3ccnc4c3CN(C)CC4)C2)C1)[C@H]1C[C@H](N2CC3(CCCO3)C2)C1]

This compound was synthesized following the procedure described for the synthesis of Example 101, starting with 1 equivalent of (1s,3s)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclobutyl 4-methylbenzenesulfonate and 5 equivalents of 6-oxa-2-azaspiro[3.4]octane. After completion, the reaction was concentrated under reduced pressure. The crude was purified by Prep HPLC (Method E) to obtain N-((1r,3r)-3-(6-oxa-2-azaspiro[3.4]octan-2-yl)cyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 19.16%; ¹H NMR (400 MHz, DMSO-d₆): δ 8.28 (s, 1H), 8.18 (d, J=5.6 Hz, 1H), 7.81-7.69 (m, 1H), 7.33-7.02 (m, 3H), 6.66 (d, J=5.8 Hz, 1H), 4.81-4.71 (m, 1H), 4.35-3.95 (m, 5H), 3.82-3.56 (m, 5H), 3.39-3.35 (m, 2H), 3.12-2.89 (m, 5H), 2.86-2.75 (m, 5H), 2.63 (br t, J=5.6 Hz, 2H), 2.37 (s, 3H), 2.30-2.20 (m, 2H), 2.15-2.06 (m, 1H), 2.02-1.88 (m, 2H), 1.87-1.78 (m, 1H), 1.74-1.52 (m, 1H), 1.48-1.28 (m, 3H), 1.09-0.89 (m, 3H); LCMS (Method C): Rt=1.40 min, m/z=698.3 [M+H]⁺; HPLC (Method C): Rt=8.46 min, 99.67%; Chiral SFC (Method E): Rt=3.27 min, 99.12%.

### Example 105. 5-Fluoro-N-isopropyl-N-((1r,3r)-3-(3-methoxyazetidin-1-yl)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 105)

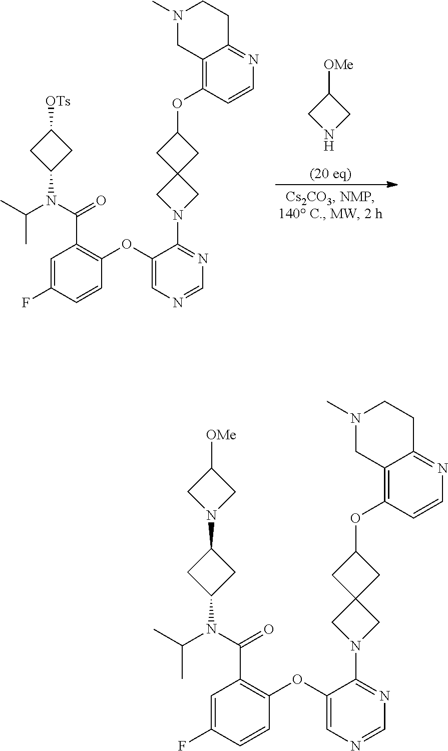 [SMILES: Cc1ccc(S(=O)(=O)O[C@H]2C[C@@H](N(C(=O)c3cc(F)ccc3Oc3cncnc3N3CC4(CC(Oc5ccnc6c5CN(C)CC6)C4)C3)C(C)C)C2)cc1 ; COC1CNC1 ; COC1CN([C@H]2C[C@H](N(C(=O)c3cc(F)ccc3Oc3cncnc3N3CC4(CC(Oc5ccnc6c5CN(C)CC6)C4)C3)C(C)C)C2)C1]

In a 5 mL microwave vial, (1s,3s)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclobutyl 4-methylbenzenesulfonate (100 mg, 0.132 mmol) and 3-methoxyazetidine (230 mg, 2.64 mmol) were dissolved in NMP (1.5 mL). To this reaction mixture, Cs₂CO₃ (129 mg, 0.396 mmol) was added at RT, and the reaction was stirred at 140° C. for 2 h in a microwave oven. The reaction progress was monitored by TLC (10% MeOH in DCM). The reaction was quenched with ice-cold water (10 mL) and the mixture was extracted with EtOAc (2×20 mL). The combined organic layer was washed with brine solution (10 mL), dried over anhydrous sodium sulfate and filtered, and the filtrate was concentrated under reduced pressure. The crude was purified by reverse phase chromatography using a SiliCycle SiliaSep C18 column (12 g) on Biotage isolera one (Mobile phase A: 10 mM NH₄HCO₃ in water, B: ACN) to obtain 5-fluoro-N-isopropyl-N-((1r,3r)-3-(3-methoxyazetidin-1-yl)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (20 mg, 22.36% yield) as a solid. ¹H NMR (400 MHz, DMSO-dh): S 8.31-8.26 (m, 1H), 8.18 (d, J=5.5 Hz, 1H), 7.79-7.64 (m, 1H), 7.34-7.03 (m, 3H), 6.66 (d, J=5.7 Hz, 1H), 4.75 (quin, J=6.4 Hz, 1H), 4.36-3.97 (m, 5H), 3.95-3.80 (m, 1H), 3.78-3.61 (m, 1H), 3.48-3.40 (m, 1H), 3.39-3.35 (m, 3H), 3.16-3.07 (m, 3H), 3.06-3.01 (m, 1H), 2.90-2.75 (m, 5H), 2.72-2.67 (m, 1H), 2.66-2.56 (m, 3H), 2.37 (s, 3H), 2.30-2.18 (m, 2H), 2.16-2.02 (m, 1H), 1.89-1.54 (m, 2H), 1.46-0.92 (m, 6H); LCMS (Method A): Rt=1.70 min, m/z=672.2 [M+H]⁺; HPLC (Method C): Rt=8.50 min, 99.25%; Chiral SFC (Method AS): Rt=3.06 min, 100%.

### Example 106. N-((1r,3r)-3-(2-Oxa-6-azaspiro[3.3]heptan-6-yl)cyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 106)

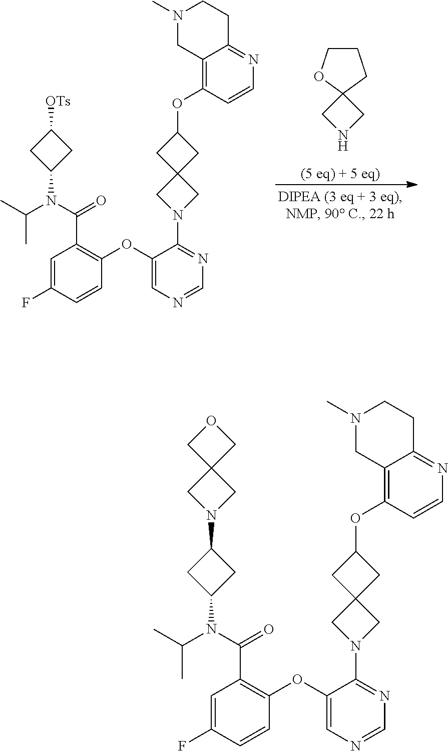 [SMILES: Cc1ccc(S(=O)(=O)O[C@H]2C[C@@H](N(C(=O)c3cc(F)ccc3Oc3cncnc3N3CC4(CC(Oc5ccnc6c5CN(C)CC6)C4)C3)C(C)C)C2)cc1 ; C1COC2(C1)CNC2 ; CC(C)N(C(=O)c1cc(F)ccc1Oc1cncnc1N1CC2(CC(Oc3ccnc4c3CN(C)CC4)C2)C1)[C@H]1C[C@H](N2CC3(COC3)C2)C1]

In 40 mL vial, (1s,3s)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclobutyl 4-methylbenzenesulfonate (200 mg, 0.264 mmol) was dissolved in NMP (4 mL). To this solution, DIPEA (0.138 mL, 0.793 mmol) and 2-oxa-6-azaspiro[3.3]heptane (131 mg, 1.321 mmol) were added at RT, and the reaction was stirred at 90° C. for 12 h. The reaction progress was monitored by LCMS. The starting material was not consumed, therefore another portion of DIPEA (102 mg, 0.793 mmol) and 2-oxa-6-azaspiro[3.3]heptane (131 mg, 1.321 mmol) were added at RT, and the reaction was continued at 90° C. for 10 h. After completion, the reaction was quenched with water (20 mL) and the mixture was extracted with EtOAc (2×25 mL). The combined organic layer was dried over anhydrous sodium sulfate and filtered, and the filtrate was concentrated under reduced pressure. The crude was purified by Prep HPLC (Method A) to obtain N-((1r,3r)-3-(2-oxa-6-azaspiro[3.3]heptan-6-yl)cyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (40 mg, 22.0% yield) as a solid. ¹H NMR (400 MHz, DMSO-d₆): δ 8.28 (s, 1H), 8.18 (d, J=5.5 Hz, 1H), 7.79-7.62 (m, 1H), 7.33-7.02 (m, 3H), 6.66 (d, J=5.5 Hz, 1H), 4.75 (quin, J=6.5 Hz, 1H), 4.59 (s, 2H), 4.53 (s, 2H), 4.38-3.91 (m, 5H), 3.82-3.59 (m, 1H), 3.18 (s, 2H), 3.09 (s, 2H), 3.02-2.92 (m, 1H), 2.87-2.72 (m, 5H), 2.66-2.60 (m, 2H), 2.37 (s, 3H), 2.30-2.20 (m, 2H), 2.16-2.00 (m, 1H), 1.88-1.48 (m, 2H), 1.45-0.86 (m, 6H), one proton merged with solvent peaks; LCMS (Method C): Rt=1.39 min, m/z=684.5 [M+H]⁺; HPLC (Method C): Rt=7.98 min, 98.61%; Chiral SFC (Method J): Rt=5.74 min, 100%.

### Example 107. N-((1r,3r)-3-(1-Oxa-6-azaspiro[3.3]heptan-6-yl)cyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 107)

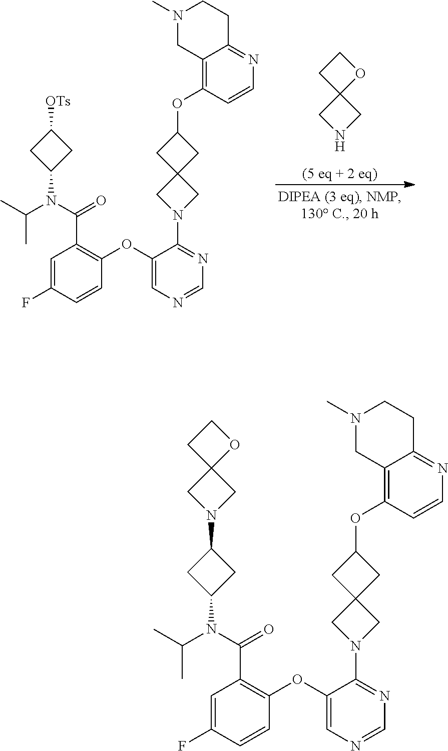 [SMILES: Cc1ccc(S(=O)(=O)O[C@H]2C[C@@H](N(C(=O)c3cc(F)ccc3Oc3cncnc3N3CC4(CC(Oc5ccnc6c5CN(C)CC6)C4)C3)C(C)C)C2)cc1 ; C1CC2(CNC2)O1 ; CC(C)N(C(=O)c1cc(F)ccc1Oc1cncnc1N1CC2(CC(Oc3ccnc4c3CN(C)CC4)C2)C1)[C@H]1C[C@H](N2CC3(CCO3)C2)C1]

In an 8 mL pressure vial, (1s,3s)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclobutyl 4-methylbenzenesulfonate (250 mg, 0.330 mmol) was dissolved in NMP (3 mL). To this solution, 1-oxa-6-azaspiro[3.3]heptane (164 mg, 1.651 mmol) and DIPEA (0.178 mL, 0.991 mmol) were added at RT and the reaction was stirred at 130° C. for 16 h. The reaction progress was monitored by TLC (10% MeOH in DCM). The starting material was not consumed, therefore another portion of 1-oxa-6-azaspiro[3.3]heptane (65.5 mg, 0.661 mmol) was added at RT and the reaction continued at 130° C. for 4 h. After completion, the reaction was quenched with aqueous NaHCO₃ solution (20 mL) and the mixture was extracted with DCM (3×30 mL). The combined organic layer was dried over anhydrous sodium sulfate and filtered, and the filtrate was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain N-((1r,3r)-3-(1-oxa-6-azaspiro[3.3]heptan-6-yl)cyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (31 mg, 13.58% yield) as a solid. ¹H NMR (400 MHz, DMSO-d₆): δ 8.28 (s, 1H), 8.19 (d, J=5.5 Hz, 1H), 7.80-7.64 (m, 1H), 7.35-7.02 (m, 3H), 6.66 (br d, J=5.4 Hz, 1H), 4.75 (quin, J=6.5 Hz, 1H), 4.47-3.91 (m, 8H), 3.81-3.61 (m, 1H), 3.47-3.42 (m, 1H), 3.37 (br s, 2H), 3.06-2.91 (m, 2H), 2.88-2.71 (m, 7H), 2.66-2.60 (m, 3H), 2.37 (s, 3H), 2.30-2.20 (m, 2H), 2.18-2.02 (m, 1H), 1.88-1.49 (m, 2H), 1.44-0.91 (m, 6H); LCMS (Method A): Rt=1.63 min, m/z=684.2 [M+H]⁺; HPLC (Method C): Rt=8.67 min, 96.85%; Chiral SFC (Method AAD): Rt=7.38 min, 100%.

### Example 108. 5-Fluoro-N-((1r,3r)-3-(3-hydroxy-3-methylazetidin-1-yl)cyclobutyl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 108)

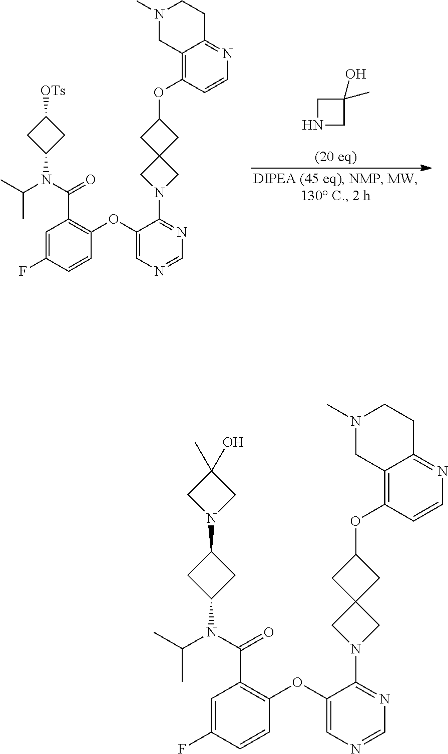

This compound was synthesized following the procedure described for the synthesis of Example 101, starting with 1 equivalent of (1s,3s)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclobutyl 4-methylbenzenesulfonate, except that 20 equivalents of 3-methylazetidin-3-ol and 45 equivalents of DIPEA were used, and the reaction was stirred at 130° C. under microwave irradiation. After completion, the reaction was concentrated under reduced pressure. The crude compound was purified by reverse phase chromatography using a SiliCycle SiliaSep C18 column (40 g) on Biotage isolera one (Mobile phase A: 10 mM NH₄HCO₃ in water, B: ACN), then by Prep HPLC (Method E) to obtain 5-fluoro-N-((1r,3r)-3-(3-hydroxy-3-methylazetidin-1-yl)cyclobutyl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 21.37%; ¹H NMR (400 MHz, DMSO-d₆): δ 8.28 (s, 1H), 8.19 (s, 1H), 7.77-7.66 (m, 1H), 7.32-7.03 (m, 3H), 6.66 (d, J=5.6 Hz, 1H), 5.10-5.02 (m, 1H), 4.81-4.67 (m, 1H), 4.33-3.95 (m, 5H), 3.83-3.62 (m, 1H), 3.16-2.92 (m, 4H), 2.88-2.71 (m, 6H), 2.63 (br t, J=5.8 Hz, 2H), 2.37 (s, 3H), 2.29-2.21 (m, 2H), 2.16-2.04 (m, 2H), 1.93-1.52 (m, 3H), 1.48-1.22 (m, 6H), 1.07-0.92 (m, 3H); LCMS (Method A): Rt=1.63 min, m/z=672.5 [M+H]⁺; HPLC (Method C): Rt=7.98 min, 99.81%; Chiral SFC (Method L): Rt=1.70 min, 100%.

### Example 109. 5-Fluoro-N-((3R,4S)-4-fluoro-1-methylpiperidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 109)

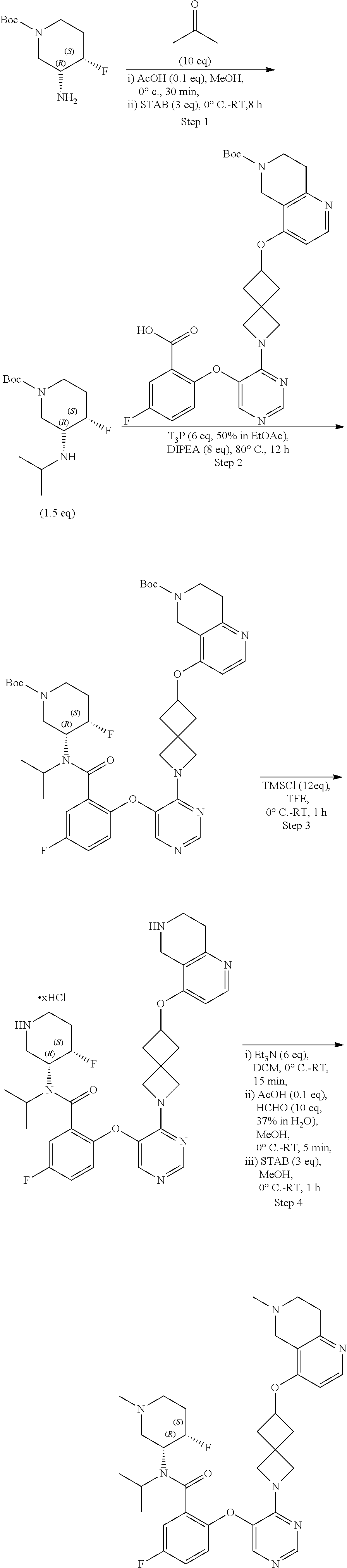

### Step 1. tert-Butyl (3R,4S)-4-fluoro-3-(isopropylamino)piperidine-1-carboxylate

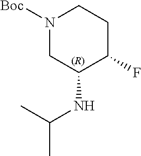

This compound was synthesized following the procedure described for the synthesis of Example 20, Step 1, starting with 1 equivalent of tert-butyl (3R,4S)-3-amino-4-fluoropiperidine-1-carboxylate, except that 10 equivalents of acetone and 3 equivalents of STAB were used. After completion, the reaction was quenched with saturated NaHCO₃, and the mixture was extracted with EtOAc. The combined organic layer was washed with brine solution, dried over anhydrous sodium sulfate, and filtered. The filtrate was concentrated under reduced pressure to obtain crude tert-butyl (3R,4S)-4-fluoro-3-(isopropylamino)piperidine-1-carboxylate as a gummy liquid. This compound was used without further purification. Quantitative yield; ¹H NMR (400 MHz, DMSO-d₆): δ 5.00-4.72 (m, 1H), 3.87-3.51 (m, 3H), 3.07-2.86 (m, 2H), 2.77-2.61 (m, 2H), 1.79-1.55 (m, 2H), 1.40 (s, 9H), 1.02-0.93 (m, 6H).

### Step 2. tert-Buyo 4-((2-(5-(2-(((3R,4S)-1-(tert-butoxycarbonyl)-4-fluoropiperidin-3-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate

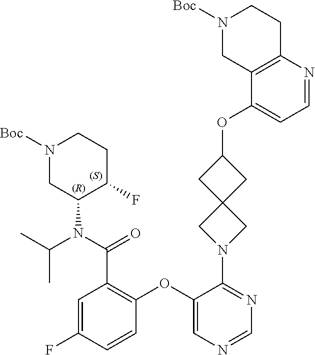

This compound was synthesized following the procedure described for the synthesis of Example 25, Step 1, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid, except that 1.5 equivalents of tert-butyl (3R,4S)-4-fluoro-3-(isopropylamino)piperidine-1-carboxylate was used. After work-up, the crude product was purified by reverse phase chromatography using a SiliCycle SiliaSep C18 column (80 g) on Biotage isolera one (Mobile phase A: 10 mM NH₄HCO₃ in water, B: ACN) to obtain tert-butyl 4-((2-(5-(2-(((3R,4S)-1-(tert-butoxycarbonyl)-4-fluoropiperidin-3-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate as a solid.

Yield: 22.91%; LCMS (Method B): Rt=1.75 min, m/z=820.3 [M+H]⁺, 97.59%.

### Step 3. 5-Fluoro-N-((3R,4S)-4-fluoropiperidin-3-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl

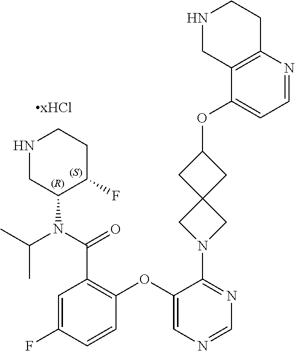

This compound was synthesized following the procedure described for the synthesis of Example 20, Step 3, starting with 1 equivalent of tert-butyl 4-((2-(5-(2-(((3R,4S)-1-(tert-butoxycarbonyl)-4-fluoropiperidin-3-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate, except that 12 equivalents of TMSCl was used. After completion, the reaction was concentrated under reduced pressure to obtain crude 5-fluoro-N-((3R,4S)-4-fluoropiperidin-3-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl as a solid. This compound was used without further purification.

Yield: 93%; LCMS (Method C): Rt=1.29 min, m/z=620.3 [M+H]⁺, 97.98%.

### Step 4. 5-Fluoro-N-((3R,4S)-4-fluoro-1-methylpiperidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

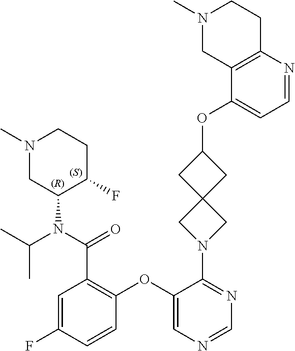

This compound was synthesized following the procedure described for the synthesis of Example 20, Step 4, starting with 1 equivalent of 5-fluoro-N-((3R,4S)-4-fluoropiperidin-3-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl, except that 6 equivalents of Et₃N, 0.1 equivalent of AcOH, and 3 equivalents of STAB were used. After work-up, the crude was purified by reverse phase chromatography using a SiliCycle SiliaSep C18 column (24 g) on Biotage isolera one (Mobile phase A: 10 mM NH₄HCO₃ in water, B: ACN) to afford 5-fluoro-N-((3R,4S)-4-fluoro-1-methylpiperidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 47.5%; ¹H NMR (400 MHz, DMSO-d₆): δ 8.34-8.30 (m, 1H), 8.18 (d, J=5.5 Hz, 1H), 7.79-7.66 (m, 1H), 7.39-7.20 (m, 2H), 7.09-6.99 (m, 1H), 6.66 (d, J=5.8 Hz, 1H), 5.07-4.88 (m, 1H), 4.81-4.67 (m, 1H), 4.34-4.05 (m, 4H), 3.85-3.67 (m, 1H), 3.62-3.45 (m, 1H), 3.39-3.35 (m, 3H), 2.86-2.73 (m, 4H), 2.66-2.61 (m, 2H), 2.45-2.40 (m, 2H), 2.37 (s, 3H), 2.30-2.21 (m, 2H), 2.19-2.08 (m, 3H), 2.07-1.97 (m, 1H), 1.90-1.78 (m, 1H), 1.52-1.29 (m, 6H), one proton merged with solvent peaks; LCMS (Method A): Rt=1.79 min, m/z=648.2 [M+H]⁺; HPLC (Method A): Rt=5.25 min, 98.74%; Chiral SFC (Method AO): Rt=1.38 min, 100%.

### Example 110. 5-Fluoro-N-((3R,4R)-4-fluoro-1-methylpiperidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 110)

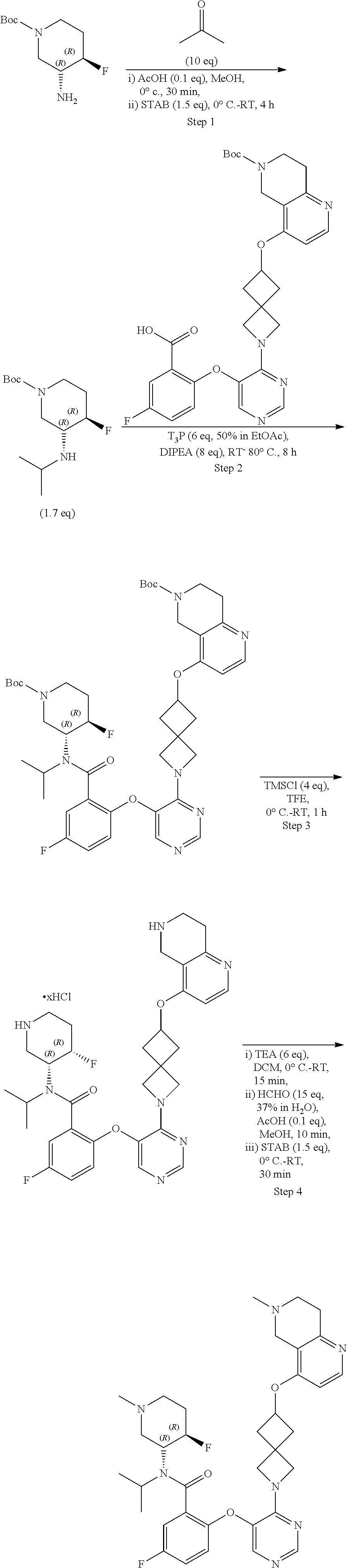

### Step 1. tert-Butyl (3R,4R)-4-fluoro-3-(isopropylamino)piperidine-1-carboxylate

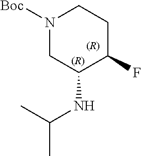

This compound was synthesized following the procedure described for the synthesis of Example 20, Step 1, starting with 1 equivalent of tert-butyl (3R,4R)-3-amino-4-fluoropiperidine-1-carboxylate, except that 10 equivalents of acetone was used. After completion, ice-cold water and aqueous Na-CO₃ were added and the mixture was extracted with EtOAc. The combined organic layer was washed with brine, dried over anhydrous sodium sulfate, and filtered. The filtrate was concentrated under reduced pressure to obtain crude tert-butyl (3R,4R)-4-fluoro-3-(isopropylamino)piperidine-1-carboxylate as a solid. This compound was used without further purification.

Yield: 67.7%; LCMS (Method A): Rt=1.86 min, m/z=261.1 [M+H]⁺, 44.18%.

### Step 2. tert-Butyl 4-((2-(5-(2-(((3R,4R)-1-(tert-butoxycarbonyl)-4-fluoropiperidin-3-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate

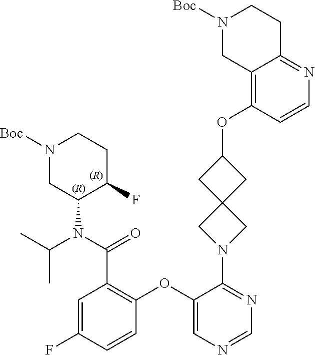

The reaction was performed following the same procedure in two batches.

This compound was synthesized following the procedure described for the synthesis of Example 25, Step 2, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.7 equivalents of tert-butyl (3R,4R)-4-fluoro-3-(isopropylamino)piperidine-1-carboxylate. The reaction was quenched with aqueous NaHCO₃ solution and the mixture was extracted with EtOAc. The combined organic layer was washed with brine, dried over anhydrous sodium sulfate and filtered, and the filtrate was concentrated under reduced pressure. The crude product was purified by reverse phase chromatography using a SiliCycle SiliaSep C18 column (80 g) on Biotage isolera one (Mobile phase A: 10 mM NH₄HCO₃ in water, B: ACN) to obtain tert-butyl 4-((2-(5-(2-(((3R,4R)-1-(tert-butoxycarbonyl)-4-fluoropiperidin-3-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate as a solid. Combined yield: 43%; LCMS (Method A): Rt=2.22 min, m/z=820.4 [M+H]⁺, 99.40%.

### Step 3. 5-Fluoro-N-((3R,4R)-4-fluoropiperidin-3-yl)-N-isopropyl-2-((4-(6-((5,6,7,8-tetrahydro-1,6-naphthyridin-4-yl)oxy)-2-azaspiro[3.3]heptan-2-yl)pyrimidin-5yl)oxy)benzamide xHCl

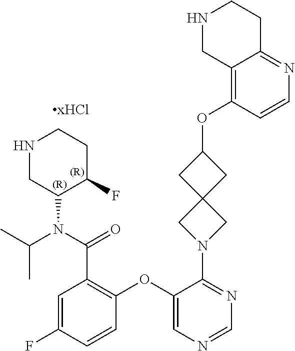

This compound was synthesized following the procedure described for the synthesis of Example 20, Step 3, starting with 1 equivalent of tert-butyl 4-((2-(5-(2-(((3R,4R)-1-(tert-butoxycarbonyl)-4-fluoropiperidin-3-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate. After completion, the reaction was concentrated under reduced pressure to obtain crude 5-fluoro-N-((3R,4R)-4-fluoropiperidin-3-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl as a solid. This material was used without further purification.

Yield: 85%; LCMS (Method A): Rt=1.55 min, m/z=620.4 [M+H]⁺, 87.08%.

### Step 4. 5-Fluoro-N-((3R4R)-4-fluoro-1-methylpiperidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

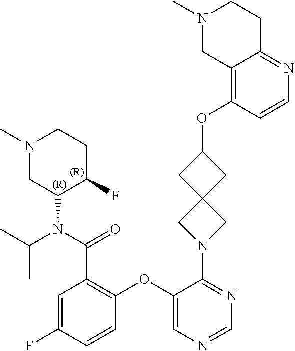

This compound was synthesized following the procedure described for the synthesis of Example 20, Step 4, starting with 1 equivalent of 5-fluoro-N-((3R,4R)-4-fluoropiperidin-3-yl)-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl, except that 15 equivalents of formaldehyde (37% in H₂O), 6 equivalents of Et₃N, 0.1 equivalent of AcOH, and 1.5 equivalents of STAB were used. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain 5-fluoro-N-((3R,4R)-4-fluoro-1-methylpiperidin-3-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 61.2%; ¹H NMR (400 MHz, DMSO-d₆): δ 8.35-8.26 (m, 1H), 8.18 (d, J=5.5 Hz, 1H), 7.84-7.65 (m, 11H), 7.43-6.93 (m, 3H), 6.66 (d, J=5.6 Hz, 1H), 5.69-5.37 (m, 1H), 4.85-4.61 (m, 1H), 4.36-4.08 (m, 4H), 3.87-3.64 (m, 1H), 3.42-3.37 (m, 2H), 2.90-2.72 (m, 5H), 2.67-2.61 (m, 2H), 2.37 (s, 3H), 2.31-2.20 (m, 3H), 2.19-2.13 (m, 2H), 2.08-1.89 (m, 3H), 1.78-1.57 (m, 1H), 1.53-0.93 (m, 6H), two protons merged with solvent peaks; LCMS (Method A): Rt=1.70 min, m/z=648.2 [M+H]⁺; HPLC (Method A): Rt=4.90 min, 98.96%; Chiral SFC (Method C): Rt=2.01 min, 98.98%.

### Example 111. N-((2S,4R)-1,2-Dimethylpiperidin-4-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 111)

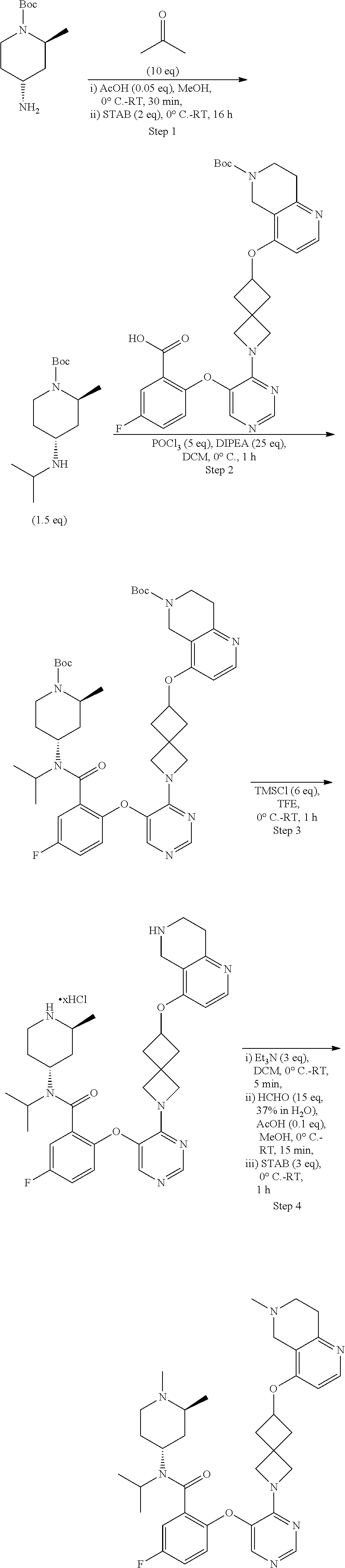

### Step 1. tert-Butyl(2S,4R)-4-(isopropylamino)-2-methylpiperidine-1-carboxylate

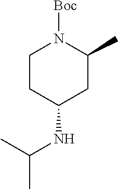 [SMILES: CC(C)N[C@@H]1CCN(C(=O)OC(C)(C)C)[C@@H](C)C1]

This compound was synthesized following the procedure described for the synthesis of Example 20, Step 1, starting with 1 equivalent of tert-butyl(2S,4R)-4-amino-2-methylpiperidine-1-carboxylate, except that 10 equivalents of acetone, 0.05 equivalent of AcOH, and 2 equivalents of STAB were used. After completion, the reaction was quenched with saturated NaHCO₃, and the mixture was extracted with EtOAc. The combined organic layer was washed with brine solution, dried over anhydrous sodium sulfate, and filtered. The filtrate was concentrated under reduced pressure to obtain crude tert-butyl(2S,4R)-4-(isopropylamino)-2-methylpiperidine-1-carboxylate as a liquid. This compound was used without further purification.

Yield: 73.1%; ¹H NMR (400 MHz, DMSO-d₆): δ 4.40-4.21 (m, 1H), 3.88-3.74 (m, 1H), 2.95-2.74 (m, 3H), 1.86-1.74 (m, 11H), 1.71-1.62 (m, 11H), 1.39 (s, 9H), 1.25-1.13 (m, 1H), 1.08 (d, J=6.3 Hz, 3H), 0.94 (d, J=6.3 Hz, 6H), two protons merged with solvent peaks; LCMS (Method A): Rt=1.49 min, m/z=257.2 [M+H]⁺, 85.55%.

### Step 2. tert-Butyl 4-((2-(5-(2-(((2S,4R)-1-(tert-butoxycarbonyl)-2-methylpiperidin-4-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate

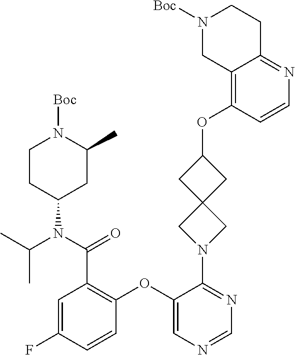 [SMILES: CC(C)N(C(=O)c1cc(F)ccc1Oc1cncnc1N1CC2(CC(Oc3ccnc4c3CN(C(=O)OC(C)(C)C)CC4)C2)C1)[C@@H]1CCN(C(=O)OC(C)(C)C)[C@@H](C)C1]

This compound was synthesized following the procedure described for the synthesis of Example 20, Step 2, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.5 equivalents of tert-butyl(2S,4R)-4-(isopropylamino)-2-methylpiperidine-1-carboxylate, except that 25 equivalents of DIPEA was used. After work-up, the crude was purified by reverse phase chromatography using a SiliCycle SiliaSep C18 column (40 g) on Biotage isolera one (Mobile phase A: 10 mM NH₄HCO₃ in water, B: ACN) to obtain tert-butyl 4-((2-(5-(2-(((2S,4R)-1-(tert-butoxycarbonyl)-2-methylpiperidin-4-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate as a solid.

Yield: 35.1%; LCMS (Method A): Rt=2.36 min, m/z=816.2 [M+H]⁺, 97.46%.

### Step 3. 5-Fluoro-N-isopropyl-N-((2S,4R)-2-methylpiperidin-4-yl)-2-((4-(6-((5, 6,7,8-tetrahydro-1,6-naphthyridin-4-yl)oxy)-2-azaspiro[3.3]heptan-2-yl)pyrimidin-5-yl)oxy)benzamide xHCl

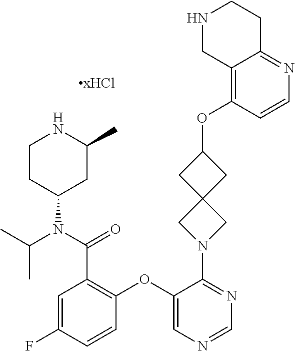 [SMILES: CC(C)N(C(=O)c1cc(F)ccc1Oc1cncnc1N1CC2(CC(Oc3ccnc4c3CNCC4)C2)C1)[C@@H]1CCN[C@@H](C)C1]

This compound was synthesized following the procedure described for the synthesis of 15 Example 20, Step 3, starting with 1 equivalent of tert-butyl 4-((2-(5-(2-(((2S,4R)-1-(tert-butoxycarbonyl)-2-methylpiperidin-4-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate, except that 6 equivalents of TMSCl was used. After completion, the reaction was concentrated under reduced pressure and the residue was triturated with EtOAc. The organic layer was decanted and the solid was dried under reduced pressure to obtain crude 5-fluoro-N-isopropyl-N-((2S,4R)-2-methylpiperidin-4-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl as a solid. This compound was used without further purification.

Yield: 89%; LCMS (Method A): Rt=1.52-1.71 min, m/z=616.2 [M+H]⁺, 71.21%.

### Step 4. N-((2S,4R)-1,2-Dimethylpiperidin-4-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

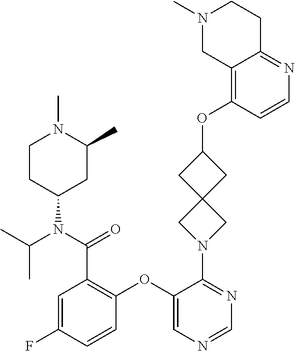 [SMILES: CC(C)N(C(=O)c1cc(F)ccc1Oc1cncnc1N1CC2(CC(Oc3ccnc4c3CN(C)CC4)C2)C1)[C@@H]1CCN(C)[C@@H](C)C1]

This compound was synthesized following the procedure described for the synthesis of Example 20, Step 4, starting with 1 equivalent of 5-fluoro-N-isopropyl-N-((2S,4R)-2-methylpiperidin-4-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl, except that 15 equivalents of formaldehyde (37% in H₂O), 3 equivalents of Et₃N, 0.1 equivalent of AcOH, and 3 equivalents of STAB were used. After work-up, the crude was purified by reverse phase chromatography using a SiliCycle SiliaSep C18 column (50 g) on Biotage isolera one (Mobile phase A: 10 mM NH₄HCO₃ in water, B: MeOH) to obtain N-((2S,4R)-1,2-dimethylpiperidin-4-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 36%; ¹H NMR (400 MHz, DMSO-d₆): δ 8.31-8.24 (m, 1H), 8.21-8.16 (m, 1H), 7.76-7.58 (m, 1H), 7.35-7.02 (m, 3H), 6.66 (d, J=5.6 Hz, 1H), 4.83-4.70 (m, 1H), 4.38-4.09 (m, 4H), 3.79-3.45 (m, 2H), 3.37 (br s, 2H), 3.11-2.93 (m, 1H), 2.85-2.74 (m, 4H), 2.63 (br t, J=5.8 Hz, 2H), 2.37 (s, 3H), 2.30-2.08 (m, 6H), 2.01-1.47 (m, 2H), 1.46-1.40 (m, 2H), 1.37-1.28 (m, 2H), 1.26-1.17 (m, 1H), 1.13-0.51 (m, 6H), one proton merged with solvent peaks; LCMS (Method A): Rt=1.75 min, m/z=644.2 [M+H]⁺; HPLC (Method A): Rt=4.66 min, 99.47%; Chiral SFC (Method AP): Rt=1.14 min, 100%.

### Example 112. N-((2R,4R)-1,2-Dimethylpiperidin-4-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 112)

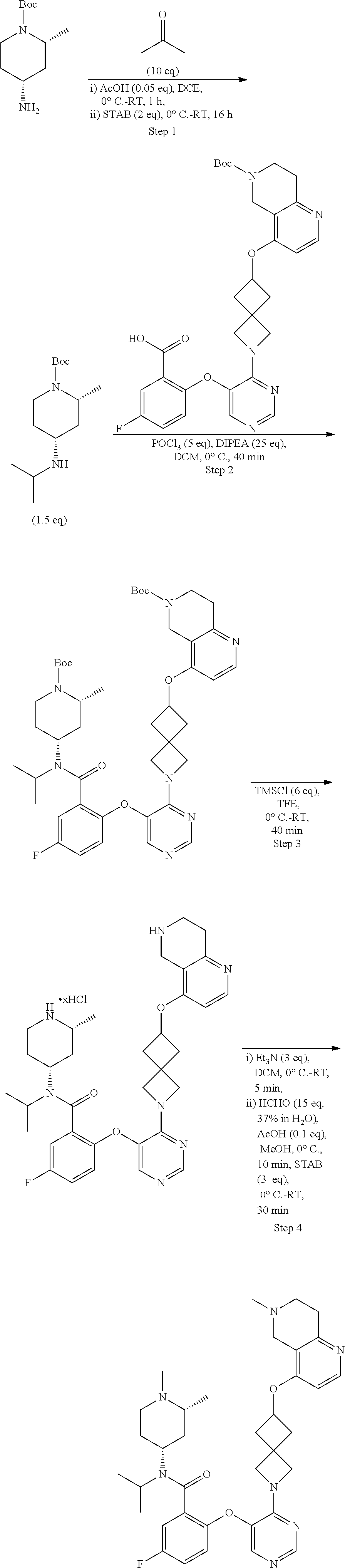

### Step 1. tert-Butyl(2R,4R)-4-(isopropylamino)-2-methylpiperidine-1-carboxylate

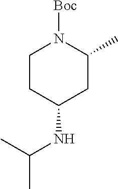 [SMILES: CC(C)N[C@@H]1CCN(C(=O)OC(C)(C)C)[C@H](C)C1]

This compound was synthesized following the procedure described for the synthesis of Example 20, Step 1, starting with 1 equivalent of tert-butyl(2R,4R)-4-amino-2-methylpiperidine-1-carboxylate, except that 10 equivalents of acetone, 0.05 equivalents of AcOH, and 2 equivalents of STAB were used. After completion, the reaction was quenched with aqueous NaHCO₃, and the mixture was extracted with 10% MeOH in DCM. The combined organic layer was dried over anhydrous sodium sulfate and filtered, and the filtrate was concentrated under reduced pressure to afford crude tert-butyl(2R,4R)-4-(isopropylamino)-2-methylpiperidine-1-carboxylate as a solid. This material was used without further purification.

Yield: 88%; ¹H NMR (400 MHz, DMSO-d₆): δ 4.00-3.88 (m, 1H), 3.58-3.51 (m, 1H), 3.21-3.11 (m, 1H), 2.99-2.91 (m, 1H), 2.89-2.79 (m, 1H), 1.93-1.89 (m, 1H), 1.74-1.58 (m, 2H), 1.47-1.42 (m, 1H), 1.39 (s, 9H), 1.23 (d, J=6.8 Hz, 3H), 0.99 (d, J=6.2 Hz, 6H), one proton merged with solvent peaks.

### Step 2. tert-Butyl 4-((2-(5-(2-(((2R,4R)-1-(tert-butoxycarbonyl)-2-methylpiperidin-4-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate

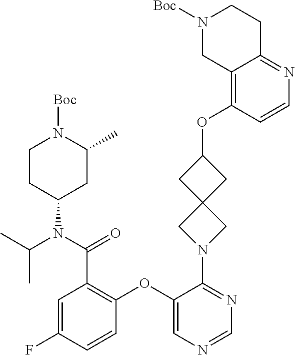 [SMILES: CC(C)N(C(=O)c1cc(F)ccc1Oc1cncnc1N1CC2(CC(Oc3ccnc4c3CN(C(=O)OC(C)(C)C)CC4)C2)C1)[C@@H]1CCN(C(=O)OC(C)(C)C)[C@H](C)C1]

This compound was synthesized following the procedure described for the synthesis of Example 20, Step 2, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.5 equivalents of tert-butyl(2R,4R)-4-(isopropylamino)-2-methylpiperidine-1-carboxylate, except that 25 equivalents of DIPEA was used. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain tert-butyl 4-((2-(5-(2-(((2R,4R)-1-(tert-butoxycarbonyl)-2-methylpiperidin-4-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate as a solid.

Yield: 29.7%; LCMS (Method A): Rt=2.60 min, m/z=816.5 [M+H]⁺, 84.01%.

### Step 3. 5-Fluoro-N-isopropyl-N-((2R,4R)-2-methylpiperidin-4-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl

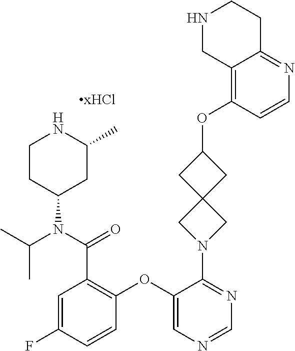 [SMILES: CC(C)N(C(=O)c1cc(F)ccc1Oc1cncnc1N1CC2(CC(Oc3ccnc4c3CNCC4)C2)C1)[C@@H]1CCN[C@H](C)C1]

This compound was synthesized following the procedure described for the synthesis of Example 20, Step 3, starting with 1 equivalent of tert-butyl 4-((2-(5-(2-(((2R,4R)-1-(tert-butoxycarbonyl)-2-methylpiperidin-4-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate, except that 6 equivalents of TMSCl was used. After completion, the reaction was concentrated under reduced pressure and the residue was triturated with EtOAc. The organic layer was decanted and the solid was dried under reduced pressure to obtain crude 5-fluoro-N-isopropyl-N-((2R,4R)-2-methylpiperidin-4-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl as a solid. This material was used without further purification.

Yield: 83%; LCMS (Method C): Rt=1.33 min, m/z=616.3 [M+H]⁺, 87.95%.

### Step 4. N-((2R,4R)-1,2-Dimethylpiperidin-4-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

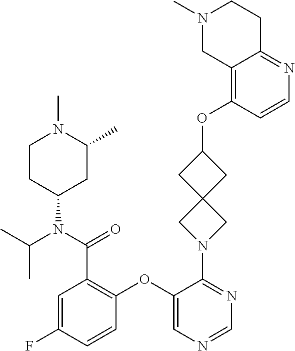 [SMILES: CC(C)N(C(=O)c1cc(F)ccc1Oc1cncnc1N1CC2(CC(Oc3ccnc4c3CN(C)CC4)C2)C1)[C@@H]1CCN(C)[C@H](C)C1]

This compound was synthesized following the procedure described for the synthesis of Example 20, Step 4, starting with 1 equivalent of 5-fluoro-N-isopropyl-N-((2R,4R)-2-methylpiperidin-4-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl, except that 10 equivalents of formaldehyde (37% in H₂O), 0.1 equivalent of AcOH, 3 equivalents of Et₃N, and 3 equivalents of STAB were used. After work-up, the crude was purified by reverse phase chromatography using a SiliCycle SiliaSep C18 column (120 g) on Biotage isolera one (Mobile phase A: 0.1% FA in water, B: ACN). The pure fractions were combined and basified with NaHCO₃, and the mixture was extracted with 10% MeOH in DCM. The combined organic layer was dried over anhydrous sodium sulfate, and filtered. The filtrate was concentrated under reduced pressure and lyophilized to obtain N-((2R,4R)-1,2-dimethylpiperidin-4-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 58.9%; ¹H NMR (400 MHz, DMSO-d₆): δ 8.32-8.25 (m, 1H), 8.18 (d, J=5.5 Hz, 1H), 7.78-7.58 (m, 1H), 7.33-7.01 (m, 3H), 6.66 (br d, J=5.5 Hz, 1H), 4.84-4.68 (m, 1H), 4.41-4.09 (m, 4H), 3.82-3.46 (m, 1H), 3.37 (s, 2H), 3.24-3.04 (m, 1H), 2.86-2.72 (m, 5H), 2.66-2.60 (m, 2H), 2.37 (s, 3H), 2.30-2.20 (m, 2H), 2.16-2.01 (m, 3H), 1.98-1.82 (m, 1H), 1.78-1.53 (m, 2H), 1.49-1.40 (m, 2H), 1.38-1.23 (m, 3H), 1.19-0.82 (m, 7H); LCMS (Method C): Rt=1.35 min, m/z=644.3 [M+H]⁺; HPLC (Method A): Rt=4.75 min, 98.34%; Chiral SFC (Method AP): Rt=1.08 min, 100%.

### Example 113. N-((3.S,6,S)-1,6-Dimethylpiperidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 113)

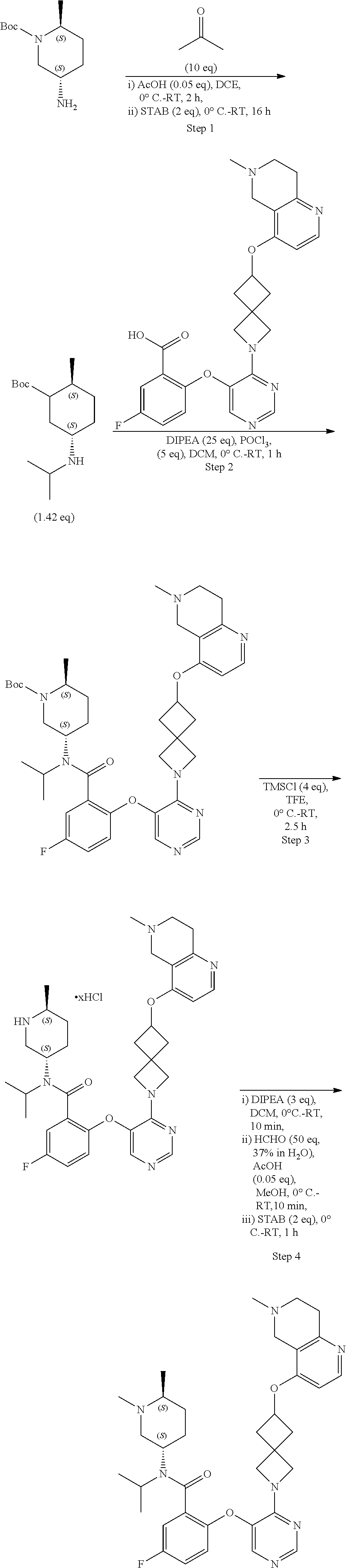

### Step 1. tert-Butyl(2S,5S)-5-(isopropylamino)-2-methylpiperidine-1-carboxylate

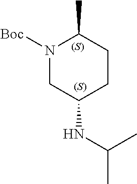

This compound was synthesized following the procedure described for the synthesis of Intermediate 1, starting with 1 equivalent of tert-butyl(2S,5S)-5-amino-2-methylpiperidine-1-carboxylate, except that 10 equivalents of acetone was used, and DCE was used as the solvent. After work-up, the organic layer was concentrated under reduced pressure to obtain crude tert-butyl (2S,5S)-5-(isopropyl amino)-2-methylpiperidine-1-carboxylate as a semi-solid. This material was used without further purification.

Yield: 56.1%; ¹H NMR (400 MHz, CDCl₃): δ 4.42-4.33 (m, 1H), 4.05-3.95 (m, 1H), 2.98-2.88 (m, 2H), 2.88-2.83 (m, 1H), 1.94-1.76 (m, 2H), 1.60-1.52 (m, 2H), 1.48 (s, 9H), 1.33-1.27 (m, 1H), 1.16 (d, J=6.8 Hz, 3H), 1.08-1.03 (m, 6H).

### Step 2. tert-Butyl(2S,5S)-5-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-2-methylpiperidine-1-carboxylate

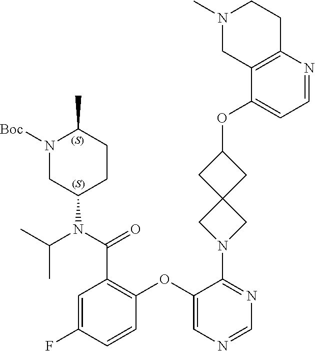

This compound was synthesized following the procedure described for the synthesis of Example 20, Step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.42 equivalents of tert-butyl(2S,5S)-5-(isopropylamino)-2-methylpiperidine-1-carboxylate, except that 25 equivalents of DIPEA and 5 equivalents of POCl₃ were used. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain tert-butyl (2S,5S)-5-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-2-methylpiperidine-1-carboxylate as a solid.

Yield: 21.47%; LCMS (Method D): Rt=2.09 min, m/z=730.2 [M+H]⁺, 94.83%.

### Step 3. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((3S,6S)-6-methylpiperidin-3-yl)benzamide xHCl

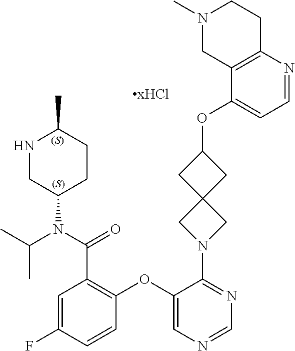

This compound was synthesized following the procedure described for the synthesis of Example 20, step 3, starting with 1 equivalent of tert-butyl(2S,5S)-5-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-2-methylpiperidine-1-carboxylate. After completion, the reaction was concentrated under reduced pressure. The residue was triturated with EtOAc, and the supernatant was decanted. The solid was dried under vacuum to obtain crude 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((3S,6S)-6-methylpiperidin-3-yl)benzamide xHCl as a solid. This material was used without further purification.

Yield: 89%; LCMS (Method A): Rt=1.73 min, m/z=630.4 [M+H]⁺, 89.89%.

### Step 4. N-((3S,6S)-1,6-Dimethylpiperidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

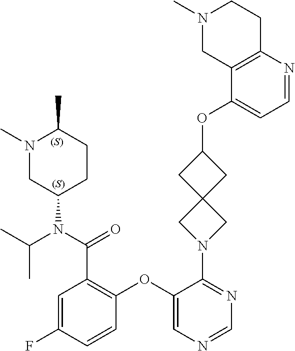

This compound was synthesized following the procedure described for the synthesis of Example 20, step 4, starting with 1 equivalent of 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((3S,6S)-6-methylpiperidin-3-yl)benzamide xHCl, except that 3 equivalents of DIPEA, 50 equivalents of formaldehyde (37% in H₂O), 0.05 equivalent of AcOH, and 2 equivalents of STAB were used. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN), then by Prep HPLC (Method M) to obtain N-((3S,6S)-1,6-dimethylpiperidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 49.1%; ¹H NMR (400 MHz, DMSO-d₆): δ 8.31-8.26 (m, 1H), 8.18 (d, J=5.6 Hz, 1H), 7.77-7.63 (m, 1H), 7.31-7.20 (m, 2H), 7.12-6.99 (m, 1H), 6.65 (br d, J=5.6 Hz, 1H), 4.81-4.69 (m, 1H), 4.33-4.09 (m, 4H), 3.77-3.49 (m, 1H), 3.26-2.94 (m, 2H), 2.87-2.72 (m, 4H), 2.66-2.58 (m, 2H), 2.37 (s, 3H), 2.31-2.20 (m, 2H), 2.19-2.00 (m, 4H), 1.87-1.53 (m, 3H), 1.49-1.19 (m, 5H), 1.12-0.87 (m, 6H), two protons merged with solvent peaks; LCMS (Method A): Rt=1.75 min, m/z=644.2 [M+H]⁺; HPLC (Method C): Rt=8.61 min, 98.90%. Chiral SFC (Method AM): Rt=4.86 min, 100%.

### Example 114. N-((3S,6R)-1,6-Dimethylpiperidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 114)

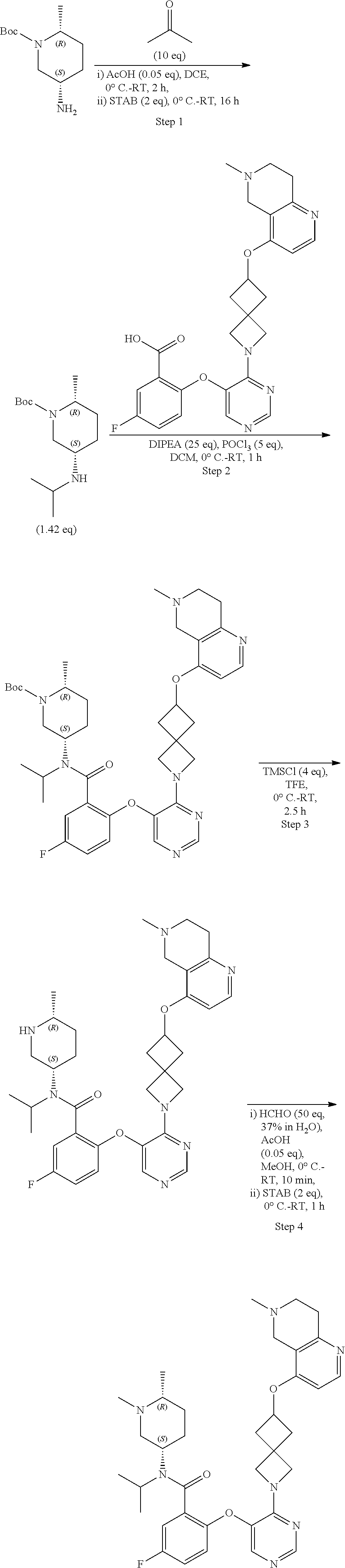

### Step 1. tert-Butyl (2R,5S)-5-(isopropylamino)-2-methylpiperidine-1-carboxylate

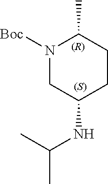

This compound was synthesized following the procedure described for the synthesis of Intermediate 1, starting with 1 equivalent of tert-butyl(2R,5ST)-5-amino-2-methylpiperidine-1-carboxylate, except that 10 equivalents of acetone was used, and DCE was used as the solvent. After work-up, the filtrate was concentrated under reduced pressure to obtain crude tert-butyl (2R,5S)-5-(isopropylamino)-2-methylpiperidine-1-carboxyl ate as a semi-solid. This material was used without further purification.

Yield: 63.9%/; ¹H NMR (400 MHz, CDCl₃): δ 4.46-4.32 (m, 1H), 4.19-4.06 (m, 1H), 3.00 (spt, J=6.2 Hz, 1H), 2.62-2.52 (m, 1H), 2.51-2.42 (m, 1H), 1.80-1.67 (m, 2H), 1.59-1.31 (m, 12H), 1.13 (d, J=6.9 Hz, 3H), 1.09-1.07 (m, 6H).

### Step 2. tert-Butyl(2R,5S)-5-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-2-methylpiperidine-1-carboxylate

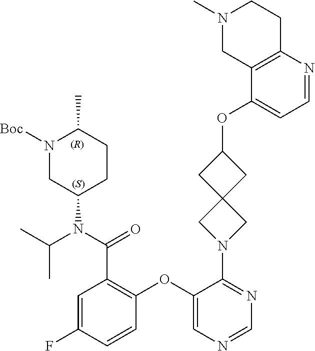

This compound was synthesized following the procedure described for the synthesis of Example 20, Step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 2 equivalents of tert-butyl(2R,5S)-5-(isopropylamino)-2-methylpiperidine-1-carboxylate, except that 5 equivalents of POCl₃ and 25 equivalents of DIPEA were used. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain tert-butyl (2R,5S)-5-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-2-methylpiperidine-1-carboxylate as a solid.

Yield: 43.7%; LCMS (Method A): Rt=2.07 min, m/z=730.3 [M+H]⁺, 85.31%.

### Step 3. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((3S,6R)-6-methylpiperidin-3-yl)benzamide

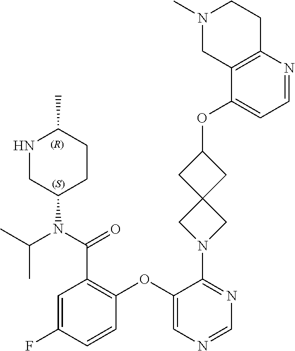

This compound was synthesized following the procedure described for the synthesis of Example 20, step 3, starting with 1 equivalent of tert-butyl(2R,5S)-5-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-2-methylpiperidine-1-carboxylate. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((3S,6R)-6-methylpiperidin-3-yl)benzamide as a solid.

Yield: 80%; LCMS (Method A): Rt=1.71 min, m/z=630.4 [M+H]⁺, 93.69%.

### Step 4. N-((3S,6R)-1,6-Dimethylpiperidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

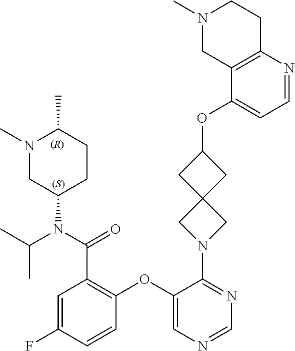

This compound was synthesized following the procedure described for the synthesis of Example 76, starting with 1 equivalent of 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((3S,6R)-6-methylpiperidin-3-yl)benzamide, except that 2 equivalents of STAB was used. After completion, the crude was purified by reverse phase chromatography using a SiliCycle SiliaSep C18 column (40 g) on Biotage isolera one (Mobile phase A: 10 mM NH₄HCO₃ in water, B: ACN) to obtain N-((3S,6R)-1,6-dimethylpiperidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 69.4%; ¹H NMR (400 MHz, DMSO-d₆): δ 8.30-8.25 (m, 1H), 8.18 (d, J=5.6 Hz, 1H), 7.75-7.63 (m, 1H), 7.30-7.21 (m, 2H), 7.14-7.02 (m, 1H), 6.68-6.62 (m, 1H), 4.80-4.70 (m, 1H), 4.37-4.09 (m, 4H), 3.77-3.60 (m, 1H), 3.36-3.32 (m, 2H), 3.29-3.15 (m, 2H), 2.87-2.74 (m, 4H), 2.66-2.59 (m, 2H), 2.36 (s, 3H), 2.30-2.03 (m, 6H), 1.83-1.63 (m, 1H), 1.56-1.31 (m, 5H), 1.22-0.82 (m, 6H), two protons merged with solvent peaks; LCMS (Method A): Rt=1.76 min, m/z=644.2 [M+H]⁺; HPLC (Method C): Rt=8.31 min, 99.22%. Chiral SFC (Method AR): Rt=2.25 min, 100%.

### Example 115. N-((3S,5R)-1,5-Dimethylpyrrolidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 115)

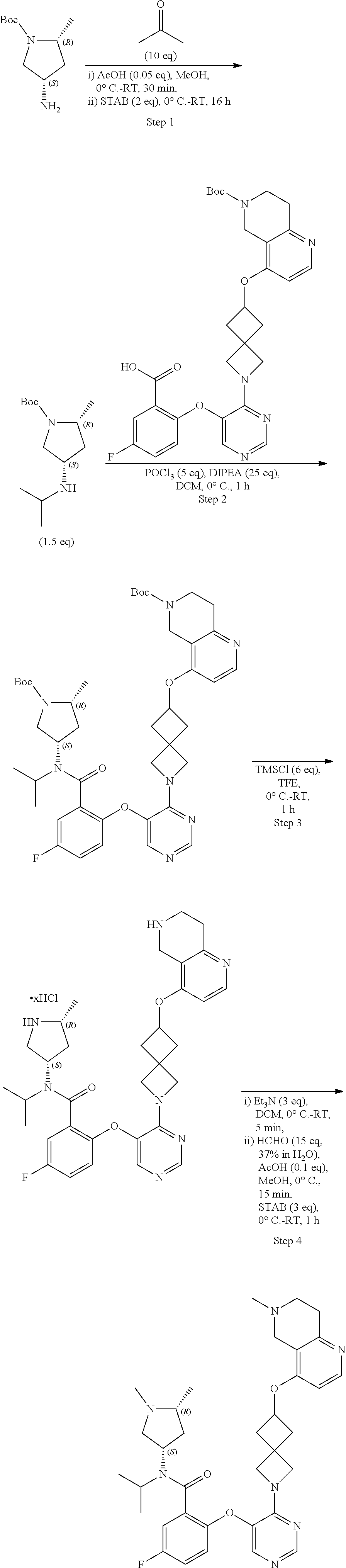

### Step 1. tert-Butyl(2R,4S)-4-(isopropylamino)-2-methylpyrrolidine-1-carboxylate

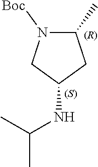

This compound was synthesized following the procedure described for the synthesis of Example 20, Step 1, starting with 1 equivalent of tert-butyl(2R,4S)-4-amino-2-methylpyrrolidine-1-carboxylate, except that 10 equivalents of acetone, 0.05 equivalent of AcOH, and 2 equivalents of STAB were used. After completion, the reaction was concentrated under reduced pressure. The residue was diluted with EtOAc and washed with aqueous NaHCO₃. The combined organic layer was dried over anhydrous sodium sulfate and filtered, and the filtrate was concentrated under reduced pressure to obtain crude tert-butyl(2R,4S)-4-(isopropylamino)-2-methylpyrrolidine-1-carboxylate as a liquid. This compound was used without further purification.

Yield: 88%; ¹H NMR (400 MHz, DMSO-d₆): δ 3.75-3.56 (m, 2H), 3.21-3.14 (m, 2H), 2.88-2.70 (m, 2H), 2.30-2.21 (m, 1H), 1.39 (s, 9H), 1.32-1.26 (m, 1H), 1.21 (d, J=6.2 Hz, 3H), 1.00-0.92 (m, 6H).

### Step 2. tert-Butyl 4-((2-(5-(2-(((3S,5R)-1-(tert-butoxycarbonyl)-5-methylpyrrolidin-3-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate

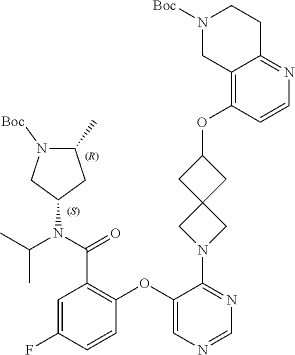

This compound was synthesized following the procedure described for the synthesis of Example 20, Step 2, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.5 equivalents of tert-butyl(2R,4S)-4-(isopropylamino)-2-methylpyrrolidine-1-carboxylate, except that 25 equivalents of DIPEA were used. After work-up, the crude was purified by reverse phase chromatography using a SiliCycle SiliaSep C18 column (40 g) on Biotage isolera one (Mobile phase A: 10 mM NH₄HCO₃ in water, B: ACN) to obtain tert-butyl 4-((2-(5-(2-(((3S,5R)-1-(tert-butoxycarbonyl)-5-methylpyrrolidin-3-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate as a solid.

Yield: 30.1%; ¹H NMR (400 MHz, DMSO-d₆): δ 8.34-8.26 (m, 1H), 8.24-8.18 (m, 1H), 7.83-7.67 (m, 1H), 7.43-6.97 (m, 3H), 6.76-6.68 (m, 1H), 4.88-4.72 (m, 1H), 4.37 (br s, 2H), 4.30-4.06 (m, 4H), 3.96-3.84 (m, 1H), 3.82-3.56 (m, 5H), 2.88-2.72 (m, 4H), 2.32-2.12 (m, 31H), 1.47-1.40 (m, 14H), 1.39-1.20 (m, 9H), 1.15-0.88 (m, 5H), one proton merged with solvent peaks; LCMS (Method C): Rt=2.07 min, m/z=802.3 [M+H]⁺, 95.96%.

### Step 3. 5-Fluoro-N-isopropyl-N-((3S,5R)-5-methylpyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl

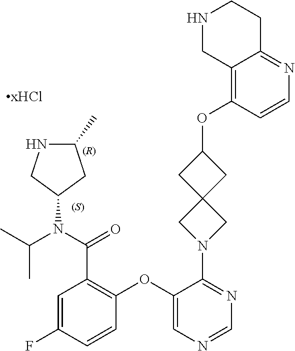

This compound was synthesized following the procedure described for the synthesis of Example 20, Step 3, starting with 1 equivalent of tert-butyl 4-((2-(5-(2-(((3S,5R)-1-(tert-butoxycarbonyl)-5-methylpyrrolidin-3-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate, except that 6 equivalents of TMSCl was used. After completion, the reaction was concentrated under reduced pressure to obtain crude 5-fluoro-N-isopropyl-N-((3S,5R)-5-methylpyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl as a solid. This compound was used without further purification.

Yield: 88%; LCMS (Method A): Rt=1.49 min, m/z=602.2 [M+H]⁺, 80.65%.

### Step 4. N-((3S,5R)-1,S-Dimethylpyrrolidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

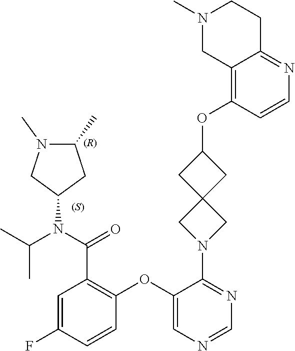

This compound was synthesized following the procedure described for the synthesis of Example 20, Step 4, starting with 1 equivalent of 5-fluoro-N-isopropyl-N-((3S,5R)-5-methylpyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl, except that 15 equivalents of formaldehyde (37% in H₂O), 3 equivalents of Et₃N, 0.1 equivalent of AcOH, and 3 equivalents of STAB were used. After work-up, the crude was purified by reverse phase chromatography using a SiliCycle SiliaSep C18 column (50 g) on Biotage isolera one (Mobile phase A: 10 mM NH₄HCO₃ in water, B: MeOH) to obtain N-((3S,5R)-1,5-dimethylpyrrolidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 39.0%; ¹H NMR (400 MHz, DMSO-d₆): δ 8.31-8.26 (m, 1H), 8.18 (d, J=5.6 Hz, 1H), 7.82-7.70 (m, 1H), 7.30-7.01 (m, 3H), 6.66 (br d, J=5.6 Hz, 1H), 4.81-4.70 (m, 1H), 4.37-4.09 (m, 4H), 4.07-3.83 (m, 2H), 3.36 (br s, 2H), 2.87-2.76 (m, 4H), 2.69-2.60 (m, 3H), 2.36 (s, 3H), 2.31-2.20 (m, 2H), 2.20-2.13 (m, 1H), 2.12-2.04 (m, 3H), 2.00-1.87 (m, 1H), 1.55-1.29 (m, 6H), 1.10-0.95 (m, 4H), one proton merged solvent peaks; LCMS (Method B): Rt=0.87 min, m/z=630.3 [M+H]⁺; HPLC (Method C): Rt=7.69 min, 98.87%; Chiral SFC (Method V): Rt=3.88 min, 98.45%.

### Example 116. N-((3S,5S)-1,5-Dimethylpyrrolidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 116)

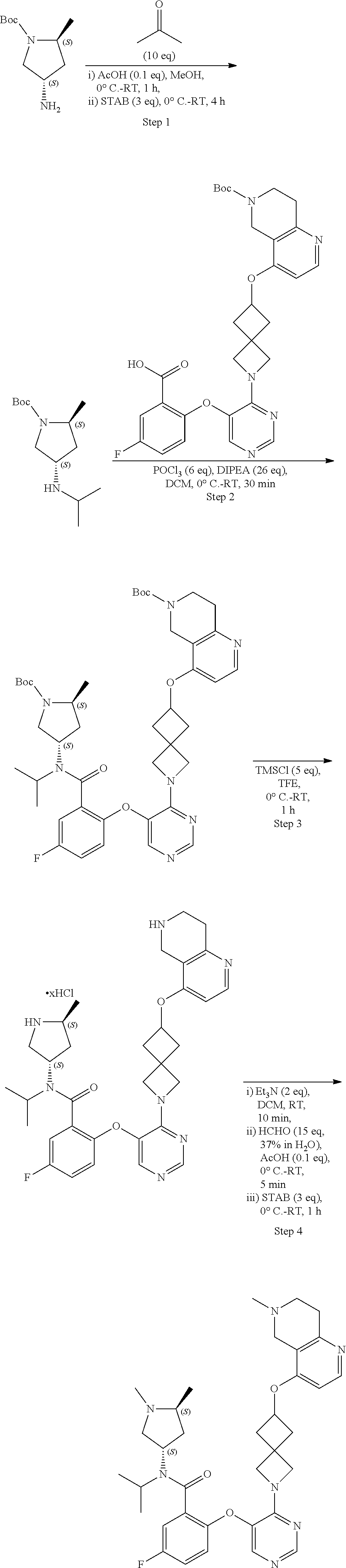

### Step 1. tert-Butyl(2S,4S)-4-(isopropylamino)-2-methylpyrrolidine-1-carboxylate

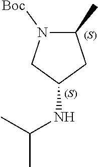

This compound was synthesized following the procedure described for the synthesis of Example 20, Step 1, starting with 1 equivalent of tert-butyl(2S,4S)-4-amino-2-methylpyrrolidine-1-carboxylate, except that 10 equivalents of acetone, and 3 equivalents of STAB were used. After completion, the reaction was quenched with saturated NaHCO₃, and the mixture was extracted with 10% MeOH in DCM. The combined organic layer was dried over anhydrous sodium sulfate and filtered, and the filtrate was concentrated under reduced pressure to obtain crude tert-butyl (2S,4S)-4-(isopropylamino)-2-methylpyrrolidine-1-carboxylate as a liquid. This compound was used without further purification.

Yield: 84%; ¹H NMR (400 MHz, DMSO-d₆): δ 3.91-3.73 (m, 1H), 3.45-3.25 (m, 2H), 2.98-2.87 (m, 1H), 2.79-2.63 (m, 1H), 1.83-1.65 (m, 2H), 1.39 (s, 9H), 1.15-1.08 (m, 3H), 1.00-0.92 (m, 6H), one proton merged with solvent peaks.

### Step 2. tert-Butyl 4-((2-(5-(2-(((3S,5S)-1-(tert-butoxycarbonyl)-5-methylpyrrolidin-3-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate

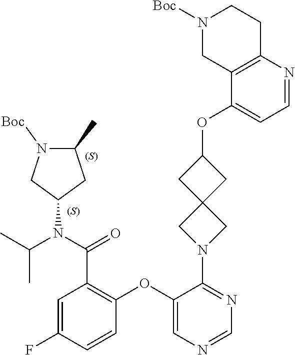

This compound was synthesized following the procedure described for the synthesis of Example 20, Step 2, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1 equivalent of tert-butyl(2S,4S)-4-(isopropylamino)-2-methylpyrrolidine-1-carboxylate. After work-up, the crude was purified by reverse phase chromatography using a SiliCycle SiliaSep C18 column (40 g) on Biotage isolera one (Mobile phase A: 10 mM NH₄HCO₃ in water, B: ACN) to obtain tert-butyl 4-((2-(5-(2-(((3S,5S)-1-(tert-butoxycarbonyl)-5-methylpyrrolidin-3-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate as a solid.

Yield: 26.8%; LCMS (Method B): Rt=1.75 min, m/z=802.6 [M+H]⁺, 92.92%.

### Step 3. 5-Fluoro-N-isopropyl-N-((3S,5S)-5-methylpyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl

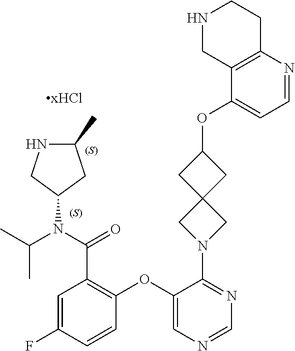

This compound was synthesized following the procedure described for the synthesis of Example 20, Step 3, starting with 1 equivalent of tert-butyl 4-((2-(5-(2-(((3S,5S)-1-(tert-butoxycarbonyl)-5-methylpyrrolidin-3-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate, except that 5 equivalents of TMSCl was used. After completion, the reaction was concentrated under reduced pressure to obtain crude 5-fluoro-N-isopropyl-N-((3S,5S)-5-methylpyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl as a solid. This material was used without further purification.

Yield: 99%; LCMS (Method A): Rt=1.46 min, m/z=602.2 [M+H]⁺, 57.82%.

### Step 4. N-((3S,5S)-1,5-Dimethylpyrrolidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

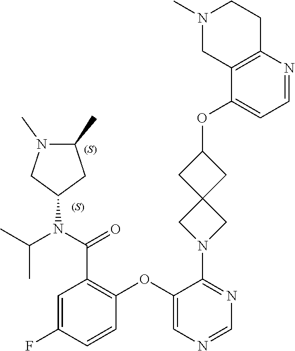

This compound was synthesized following the procedure described for the synthesis of Example 20, Step 2, starting with 1 equivalent of 5-fluoro-N-isopropyl-N-((3S,5S)-5-methylpyrrolidin-3-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl, except that 15 equivalents of formaldehyde (37% in H₂O), 0.1 equivalent of AcOH, and 3 equivalents of STAB were used. After work-up, the crude was purified by reverse phase chromatography using a SiliCycle SiliaSep C18 column (40 g) on Biotage isolera one (Mobile phase A: 10 mM NH₄HCO₃ in water, B: ACN) to obtain N-((3S,5S)-1,5-dimethylpyrrolidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 40.1%; ¹H NMR (400 MHz, DMSO-d₆): δ 8.32-8.25 (m, 1H), 8.18 (d, J=5.6 Hz, 11H), 7.80-7.70 (m, 11H), 7.32-7.17 (m, 2H), 7.14-7.02 (m, 1H), 6.69-6.62 (m, 1H), 4.81-4.71 (m, 1H), 4.37-4.07 (m, 4H), 4.01-3.64 (m, 2H), 3.36 (s, 2H), 2.84-2.72 (m, 6H), 2.66-2.60 (m, 2H), 2.36 (s, 3H), 2.31-2.10 (m, 6H), 1.66-1.42 (m, 2H), 1.40-1.28 (m, 1H), 1.12-0.88 (m, 8H); LCMS (Method A): Rt=1.69 min, m/z=630.2 [M+H]⁺; HPLC (Method C): Rt=8.19 min, 98.78%; Chiral SFC (Method AS): Rt=2.74 min, 100%.

### Example 117. N-((1S,3S)-3-(Dimethylamino)cyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 117)

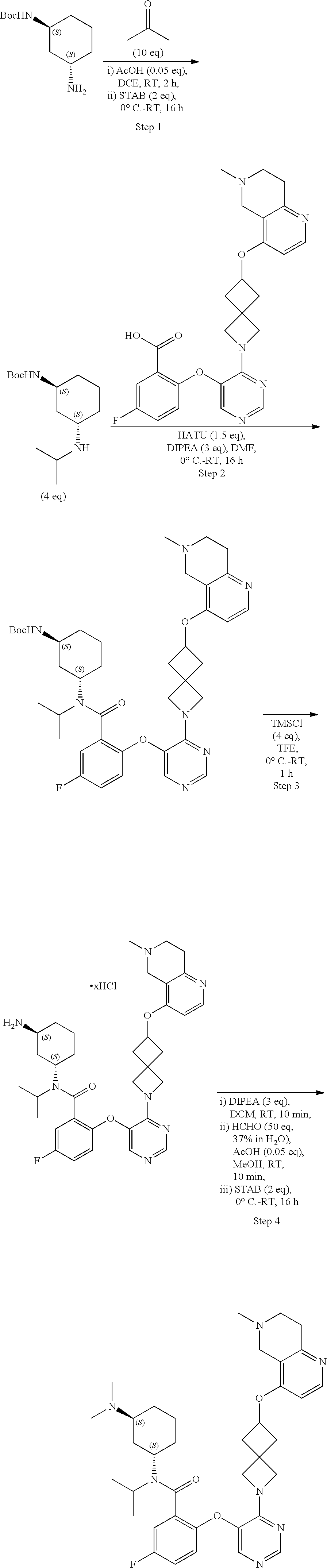

### Step 1. tert-Butyl ((1S,3S)-3-(isopropylamino)cyclohexyl)carbamate

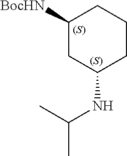

This compound was synthesized following the procedure described for the synthesis of Intermediate 1, starting with 1 equivalent of tert-butyl ((1S,3S)-3-aminocyclohexyl)carbamate, except that 10 equivalents of acetone was used, and DCE was used as the solvent. After work-up, the filtrate was concentrated under reduced pressure to obtain crude tert-butyl ((1S,3S)-3-(isopropylamino)cyclohexyl)carbamate as a semi-solid. This material was used without further purification.

Yield: 64.4%; ¹H NMR (400 MHz, DMSO-d₆): δ 4.55 (br s, 1H), 3.87 (br s, 1H), 2.95 (spt, J=6.2 Hz, 1H), 2.89-2.81 (m, 1H), 1.80-1.61 (m, 4H), 1.57-1.50 (m, 2H), 1.47 (s, 9H), 1.43-1.24 (m, 2H), 1.07-1.04 (m, 6H), one proton merged with solvent peaks; LCMS-ELSD (Method A): Rt=1.41 min, m/z=257.2 [M+H]⁺, 75.38%.

### Step 2. tert-Butyl ((1S,3S)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclohexyl)carbamate

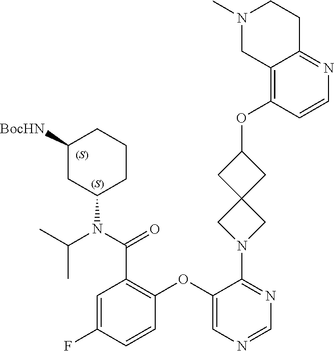

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 4 equivalents of tert-butyl ((1S,3S)-3-(isopropylamino)cyclohexyl)carbamate. After work-up the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl ((1S,3S)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclohexyl)carbamate as a solid.

Yield: 31.6%; LCMS (Method A): Rt=2.01 min, m/z=730.5 [M+H]⁺, 93.88%.

### Step 3. N-((1S,3S)-3-Aminocyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl

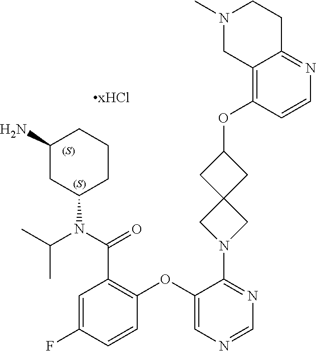

This compound was synthesized following the procedure described for the synthesis of Example 20, step 3, starting with 1 equivalent of tert-butyl ((1S,3S)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclohexyl)carbamate. After completion, the reaction was concentrated under reduced pressure. The residue was triturated with EtOAc, and the supernatant was decanted. The solid was dried under vacuum to obtain crude N-((1S,3S)-3-aminocyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl. This material was used without further purification.

Yield: 85%; LCMS (Method C): Rt=1.33 min, m/z=630.3 [M+H]⁺, 89.41%.

### Step 4. N-((1S,3S)-3-(Dimethylamino)cyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

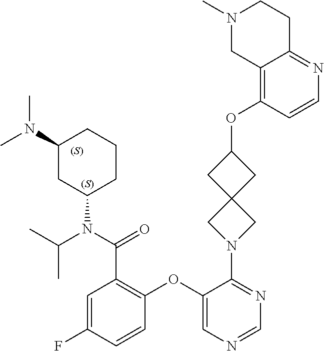

This compound was synthesized following the procedure described for the synthesis of Example 20, step 4, starting with 1 equivalent of N-((1S,3S)-3-aminocyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl, except that 3 equivalents of DIPEA, 50 equivalents of formaldehyde (37% in H₂O), 0.05 equivalent of AcOH, and 2 equivalents of STAB were used. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN), then by Prep HPLC (Method M) to obtain N-((1S,3S)-3-(dimethylamino)cyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 28.1%; ¹H NMR (400 MHz, DMSO-d₆): δ 8.28-8.26 (m, 1H), 8.18 (d, J=5.5 Hz, 1H), 7.74-7.58 (m, 1H), 7.33-7.20 (m, 2H), 7.14-7.04 (m, 1H), 6.66 (d, J=5.7 Hz, 1H), 4.80-4.70 (m, 1H), 4.33-4.09 (m, 4H), 3.74-3.63 (m, 1H), 3.63-3.43 (m, 1H), 3.37 (br s, 2H), 2.85-2.74 (m, 4H), 2.63 (br t, 1=5.6 Hz, 2H), 2.37 (s, 3H), 2.29-2.21 (m, 2H), 2.20-1.97 (m, 5H), 1.96-1.85 (m, 4H), 1.77-1.51 (m, 3H), 1.50-0.91 (m, 9H); LCMS (Method A): Rt=1.91 min, m/z=658.2 [M+H]⁺, HPLC (Method A): Rt=5.31 min, 99.53%; Chiral SFC (Method AAH): Rt=1.88 min, 100%.

### Example 118. N-((1S,3R)-3-(Dimethylamino)cyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 118)

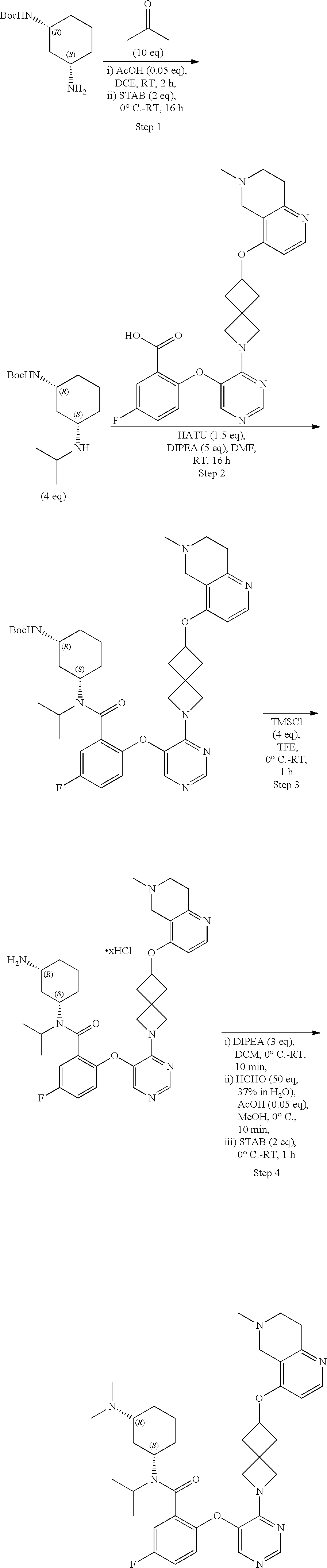

### Step 1. tert-Butyl ((1R,3R)-3-(isopropylamino)cyclohexyl)carbamate

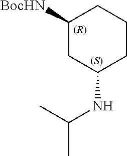

This compound was synthesized following the procedure described for the synthesis of Intermediate 1, starting with 1 equivalent of tert-butyl ((1R,3S)-3-aminocyclohexyl)carbamate, except that 10 equivalents of acetone was used, and DCE was used as the solvent. After work-up, the filtrate was concentrated under reduced pressure to obtain crude tert-butyl ((1R,3S)-3-(isopropylamino)cyclohexyl)carbamate as a semi-solid. This material was used without further purification.

Yield: 87%; ¹H NMR (400 MHz, CDCl₃): δ 4.72 (br s, 1H), 3.60-3.34 (m, 1H), 2.95 (spt, J=6.2 Hz, 1H), 2.69-2.62 (m, 11-H), 1.92-1.74 (m, 4H), 1.45 (s, 9H1), 1.40-1.25 (m, 2H), 1.05 (d, J=6.3 Hz, 6H), 1.02-0.89 (m, 2H), one proton merged with solvent peak.

### Step 2. tert-Butyl ((R, 3S)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclohexyl)carbamate

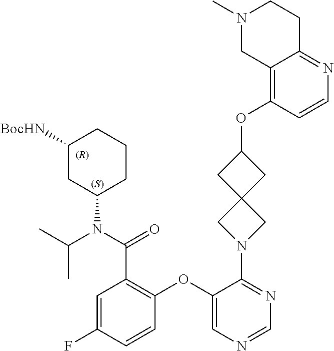

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 4 equivalents of tert-butyl ((1R,3S)-3-(isopropylamino)cyclohexyl)carbamate, except that 5 equivalents of DIPEA was used. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl ((1R,3S)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclohexyl)carbamate as a solid.

Yield: 52%; LCMS (Method A): Rt=1.98 min, m/z=730.3 [M+H]⁺, 96.54%.

### Step 3. N-((1S,3R)-3-Aminocyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl

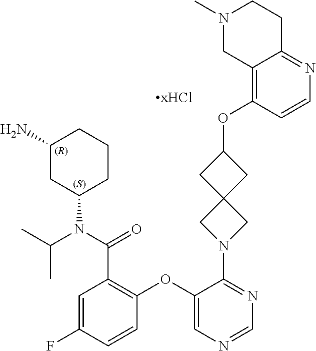

This compound was synthesized following the procedure described for the synthesis of Example 20, step 3, starting with 1 equivalent of tert-butyl ((1R,3S)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclohexyl)carbamate. After completion, the reaction was concentrated under reduced pressure. The residue was triturated with EtOAc, and the supernatant was decanted. The solid was dried under vacuum to obtain crude N-((1S,3R)-3-aminocyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl. This material was used without further purification.

Yield: 92%; LCMS (Method C): Rt=1.34 min, m/z=630.3 [M+H]⁺, 93.26%.

### Step 4. N-((1S,3R)-3-(Dimethylamino)cyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

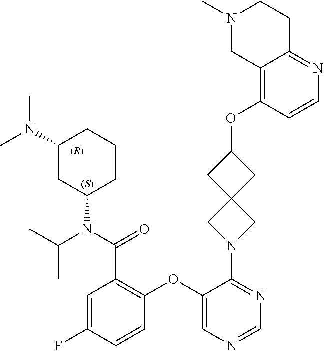

This compound was synthesized following the procedure described for the synthesis of Example 20, step 4, starting with 1 equivalent of N-((1S,3R)-3-aminocyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl, except that 3 equivalents of DIPEA, 50 equivalents of formaldehyde (37% in H₂O), 0.05 equivalent of AcOH, and 2 equivalents of STAB were used. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN), then by Prep HPLC (Method M) to obtain N-((1S,3R)-3-(dimethylamino)cyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 50.7%; ¹H NMR (400 MHz, DMSO-d₆): δ 8.28-8.26 (m, 1H), 8.18 (d, J=5.5 Hz, 1H), 7.74-7.64 (m, 1H), 7.32-7.20 (m, 2H), 7.13-7.04 (mn, 1H), 6.67-6.64 (mn, 1H), 4.80-4.70 (m, 1H), 4.33-4.09 (m, 4H), 3.78-3.53 (m, 1H), 3.37 (br s, 2H), 3.26-3.03 (m, 2H), 2.86-2.73 (m, 4H), 2.66-2.60 (m, 2H), 2.37 (s, 3H), 2.31-2.21 (m, 4H), 2.20-02.04 (m, 6H), 1.83-1.55 (m, 3H), 1.54-0.90 (m, 9H); LCMS (Method C): Rt=1.35 min, mn/z=658.3 [M+H]⁺; HPLC (Method B): Rt=2.77 min, 99.11%; SFC (Method AI): Rt=2.18 min, 100%.

### Example 119. (R)-5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(2-methyl-5-oxa-2-azaspiro[3.4]octan-7-yl)benzamide (Compound No. 119)

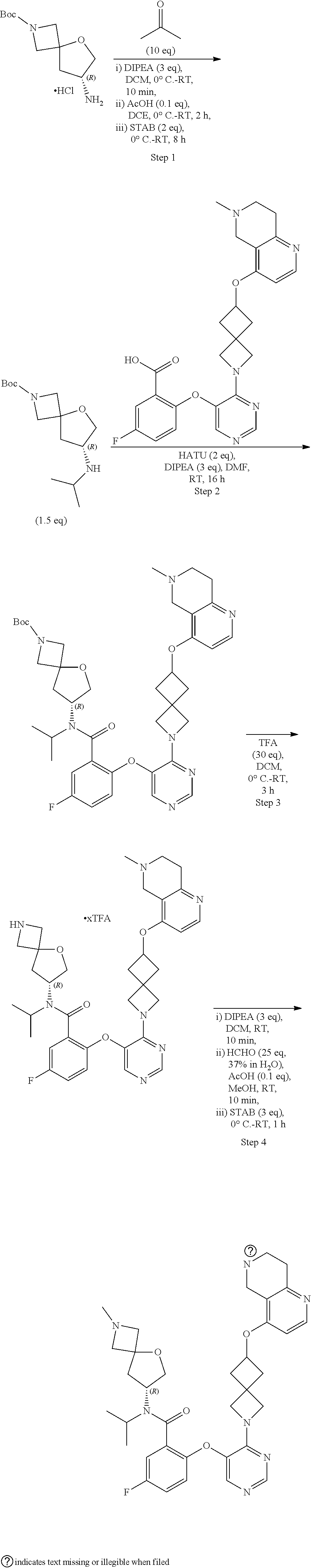

### Step 1. tert-Butyl (R)-7-(isopropylamino)-5-oxa-2-azaspiro[3.4]octane-2-carboxylate

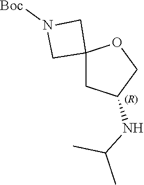

To a stirred solution of tert-butyl (R)-7-amino-5-oxa-2-azaspiro[3.4]octane-2-carboxylate hydrochloride (1 g, 3.78 mmol) in DCM (15 mL), DIPEA (1.974 mL, 11.33 mmol) was added at 0° C., and the reaction was stirred at RT for 10 min. The reaction was concentrated under reduced pressure, and the residue was dissolved in DCE (15 mL). To this reaction mixture, acetone (2.80 mL, 37.8 mmol) and acetic acid (0.022 mL, 0.378 mmol) were added at 0° C., and the reaction was stirred at RT for 2 h. The reaction was cooled to 0° C., STAB (1.601 g, 7.55 mmol) was added, and the reaction was stirred at RT for 8 h. The reaction progress was monitored by TLC (10% MeOH in DCM). After completion, saturated sodium bicarbonate solution (50 mL) was added and the mixture was extracted with 3% MeOH in DCM (3×50 mL). The combined organic layer was dried over anhydrous sodium sulfate and filtered, and the filtrate was concentrated under reduced pressure to obtain crude tert-butyl (R)-7-(isopropylamino)-5-oxa-2-azaspiro[3.4]octane-2-carboxylate (960 mg, 85% yield) as a semi-solid. This material was used without further purification. ¹H NMR (400 MHz, DMSO-d₆): δ 3.91-3.74 (m, 4H), 3.44-3.39 (m, 1H), 3.39-3.34 (m, 1H), 2.75-2.65 (m, 1H), 2.24-2.12 (m, 1H), 1.95-1.85 (m, 1H), 1.54 (br s, 1H), 1.37 (s, 9H), 1.01-0.90 (m, 6H), one proton merged with solvent peaks; LCMS (Method A): Rt=1.63 min, m/z=271.1 [M+H]⁺, 90.79%.

### Step 2. tert-Butyl (R)-7-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-oxa-2-azaspiro[3.4]octane-2-carboxylate

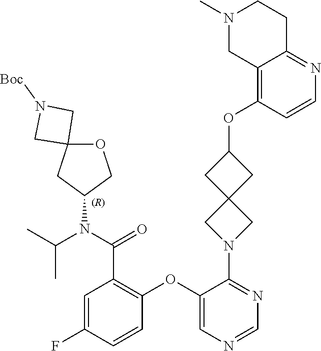

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.5 equivalents of tert-butyl (R)-7-(isopropylamino)-5-oxa-2-azaspiro[3.4]octane-2-carboxylate, except that 2 equivalents of HATU was used. After work-up the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl (R)-7-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-oxa-2-azaspiro[3.4]octane-2-carboxylate as a solid.

Yield: 31.4%; ¹H NMR (400 MHz, DMSO-d₆): δ 8.29 (d, J=9.4 Hz, 1H), 8.17 (t, J=4.6 Hz, 1H), 7.83-7.73 (m, 1H), 7.37-7.20 (m, 2H), 7.11-7.01 (m, 1H), 6.69-6.64 (m, 1H), 4.79-4.69 (m, 1H), 4.34-4.15 (m, 3H), 4.14-4.01 (m, 3H), 4.00-3.66 (m, 6H), 3.35 (br s, 2H), 2.86-2.72 (m, 4H), 2.62 (br t, J=5.3 Hz, 2H), 2.36 (s, 3H), 2.32-2.20 (m, 3H), 1.48-1.26 (m, 10H), 1.16-0.88 (m, 6H); LCMS (Method A): Rt=1.92 min, m/z=744.2 [M+H]⁺, 99.15%.

### Step 3. (R)-5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(5-oxa-2-azaspiro[3.4]octan-7-yl)benzamide xTFA

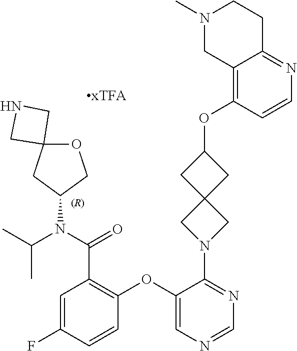

In a 25 mL round bottom flask under a nitrogen atmosphere, tert-butyl (R)-7-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-oxa-2-azaspiro[3.4]octane-2-carboxylate (250 mg, 0.336 mmol) was dissolved in DCM (5 mL). To this solution, TFA (0.78 mL, 10.08 mmol) was added, and the reaction was stirred at RT for 3 h. The reaction progress was monitored by LCMS. After completion, the reaction was concentrated under reduced pressure to obtain crude (R)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(5-oxa-2-azaspiro[3.4]octan-7-yl)benzamide xTFA (240 mg, 89% yield) as a solid. This material was used without further purification.

Yield: 89%; LCMS (Method C): Rt=1.32 min, m/z=644.3 [M+H]⁺, 94.75%.

### Step 4. (R)—5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(2-methyl-5-oxa-2-azaspiro[3.4]octan-7-yl)benzamide

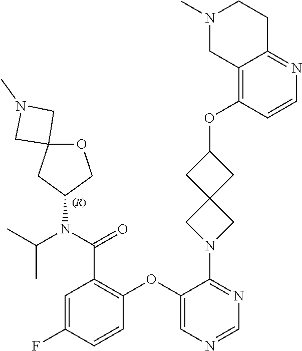

This compound was synthesized following the procedure described for the synthesis of Example 20, step 4, starting with 1 equivalent of (R)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(5-oxa-2-azaspiro[3.4]octan-7-yl)benzamide xTFA, except that 3 equivalents of DIPEA, 25 equivalents of formaldehyde (37% in H₂O), 0.1 equivalent of AcOH, and 3 equivalents of STAB were used. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain (R)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(2-methyl-5-oxa-2-azaspiro[3.4]octan-7-yl)benzamide as a solid.

Yield: 75%; ¹H NMR (400 MHz, DMSO-d₆): δ 8.33-8.27 (m, 1H), 8.20-8.15 (m, 1H), 7.84-7.73 (m, 1H), 7.35-7.20 (m, 2H), 7.10-6.98 (m, 1H), 6.68-6.62 (m, 1H), 4.80-4.70 (m, 1H), 4.34-4.04 (m, 5H), 4.04-3.87 (m, 1H), 3.82-3.63 (m, 2H), 3.56-3.39 (m, 2H), 3.22-3.02 (m, 2H), 2.85-2.69 (m, 5H), 2.66-2.58 (m, 2H), 2.36 (s, 3H), 2.30-2.11 (m, 6H), 1.48-0.92 (m, 6H), two protons merged with solvent peaks; LCMS (Method A): Rt=1.74 min, m/z=658.2 [M+H]⁺; HPLC (Method A): Rt=4.49 m, 98.67%; Chiral SFC (Method: Rt=5.92 mn, 97.50%.

### Example 120. (S)-5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(2-methyl-5-oxa-2-azaspiro[3.4]octan-7-yl)benzamide (Compound No. 120)

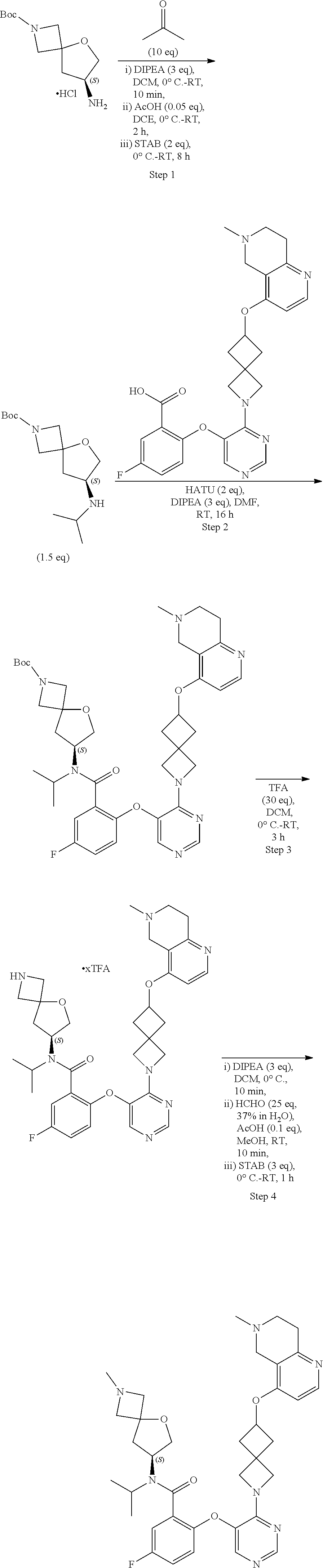

### Step 1. tert-Butyl (S)-7-(isopropylamino)-5-oxa-2-azaspiro[3.4]octane-2-carboxylate

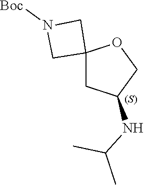

This compound was synthesized following the procedure described for the synthesis of Example 119, step 1, starting with 1 equivalent of tert-butyl (S)-7-amino-5-oxa-2-azaspiro[3.4]octane-2-carboxylate hydrochloride, except that 0.05 equivalent of acetic acid was used. After work-up, the filtrate was concentrated under reduced pressure to obtain tert-butyl (S)-7-(isopropylamino)-5-oxa-2-azaspiro[3.4]octane-2-carboxylate as a semi-solid. This material was used without further purification.

Yield: 94%; ¹H NMR (400 MHz, DMSO-d₆): δ 3.92-3.74 (m, 4H), 3.44-3.39 (m, 1H), 3.39-3.35 (m, 1H), 2.74-2.68 (m, 1H), 2.22-2.13 (m, 1H), 1.93-1.85 (m, 1H), 1.53 (br s, 1H), 1.37 (s, 9H), 0.99-0.92 (m, 6H), one proton merged with solvent peaks; LCMS (Method A): Rt=1.59 min, m/z=271.1 [M+H]⁺, 96.05%.

### Step 2. tert-Butyl (S)-7-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-oxa-2-azaspiro[3.4]octane-2-carboxylate

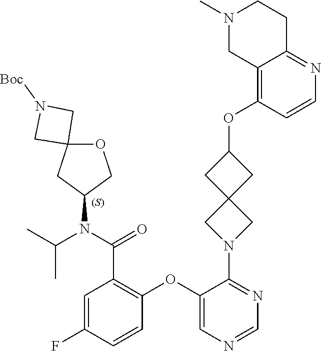

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.5 equivalents of tert-butyl (S)-7-(isopropylamino)-5-oxa-2-azaspiro[3.4]octane-2-carboxylate, except that 2 equivalents of HATU was used. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl (S)-7-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-oxa-2-azaspiro[3.4]octane-2-carboxylate as a solid.

Yield: 31.4%; ¹H NMR (400 MHz, DMSO-dh): 6 8.29 (d, J=9.4 Hz, 1H), 8.17 (t, J=4.6 Hz, 1H), 7.84-7.73 (m, 1H), 7.38-7.20 (m, 2H), 7.11-7.00 (m, 1H), 6.66 (d, J=5.5 Hz, 1H), 4.80-4.68 (m, 1H), 4.34-4.15 (m, 3H), 4.13-4.02 (m, 3H), 4.00-3.67 (m, 6H), 3.35 (br s, 2H), 2.86-2.72 (m, 4H), 2.62 (br t, J=5.3 Hz, 2H), 2.36 (s, 3H), 2.31-2.22 (m, 3H), 1.49-1.28 (m, 10H), 1.16-0.91 (m, 6H); LCMS (Method A): Rt=1.92 min, m/z=744.1 [M+H]⁺, 96.98%.

### Step 3. (S)—5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(5-oxa-2-azaspiro[3.4]octan-7-yl)benzamide xTFA

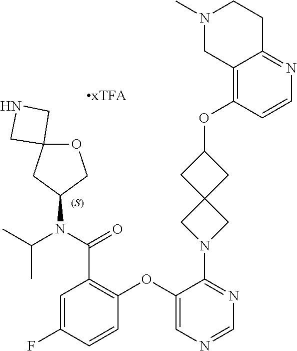

This compound was synthesized following the procedure described for the synthesis of Example 119, step 3, starting with 1 equivalent of tert-butyl (S)-7-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-oxa-2-azaspiro[3.4]octane-2-carboxylate. After completion, the reaction was concentrated under reduced pressure to obtain crude (S)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(5-oxa-2-azaspiro[3.4]octan-7-yl)benzamide xTFA as a solid. This material was used without further purification.

Yield: 95%; LCMS (Method C): Rt=1.31 min, m/z=644.3 [M+H]⁺, 96.69%.

### Step 4. (S)—5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(2-methyl-5-oxa-2-azaspiro[3.4]octan-7-yl)benzamide

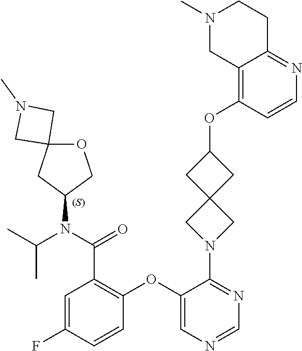

This compound was synthesized following the procedure described for the synthesis of Example 20, step 4, starting with 1 equivalent of (S)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(5-oxa-2-azaspiro[3.4]octan-7-yl)benzamide xTFA, except that 3 equivalents of DIPEA, 25 equivalents of formaldehyde (37% in H₂O), 0.1 equivalent of AcOH, and 3 equivalents of STAB were used. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain (,S)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(2-methyl-5-oxa-2-azaspiro[3.4]octan-7-yl)benzamide as a solid.

Yield: 72.5%; ¹H NMR (400 MHz, DMSO-d₆): δ 8.32-8.27 (m, 1H), 8.20-8.16 (m, 1H), 7.85-7.73 (m, 1H), 7.35-7.20 (m, 2H), 7.10-6.99 (m, 1H), 6.69-6.63 (m, 1H), 4.80-4.70 (m, 1H), 4.36-4.04 (m, 5H), 4.03-3.88 (m, 1H), 3.80-3.64 (m, 2H), 3.55-3.38 (m, 2H), 3.15-2.99 (m, 2H), 2.84-2.70 (m, 5H), 2.66-2.59 (m, 2H), 2.36 (s, 3H), 2.30-2.10 (m, 6H), 1.48-0.92 (m, 6H), two protons merged with solvent peaks; LCMS (Method A): Rt=1.74 min, m/z=658.2 [M+H]⁺; HPLC (Method A): Rt=4.48 min, 99.88%; Chiral SFC (Method C): Rt=3.79 min, 98.97%.

### Example 121. N-((1s,4s)-4-(Dimethylamino)-4-methylcyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 121)

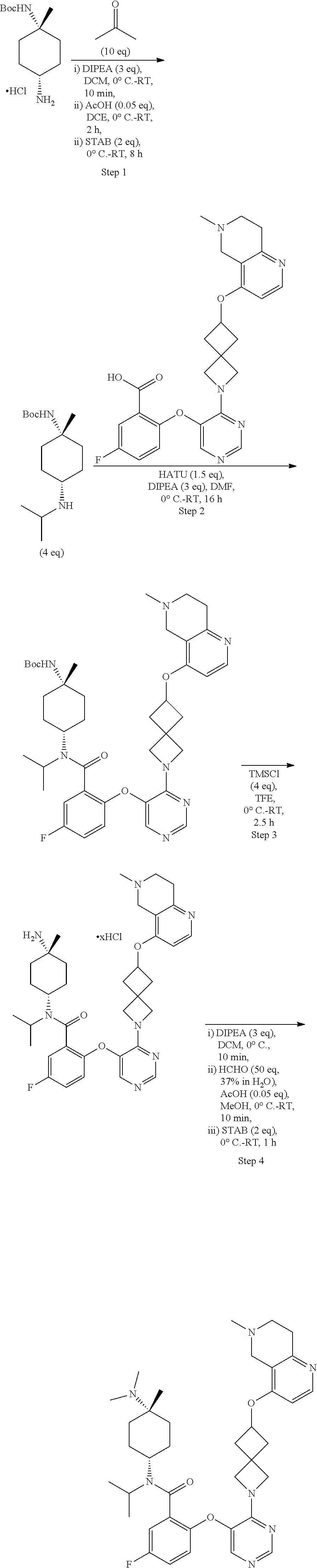

### Step 1. tert-Butyl ((1s,4s)-4-(isopropylamino)-1-methylcyclohexyl)carbamate

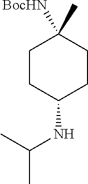 [SMILES: CC(C)N[C@H]1CC[C@@](C)(NC(=O)OC(C)(C)C)CC1]

This compound was synthesized following the procedure described for the synthesis of Example 119, step 1, starting with 1 equivalent of tert-butyl ((1s,4s)-4-amino-1-methylcyclohexyl)carbamate hydrochloride, except that 0.05 equivalent of AcOH was used. After work-up, the filtrate was concentrated under reduced pressure to obtain crude tert-butyl ((1s,4s)-4-(isopropylamino)-1-methylcyclohexyl)carbamate (930 mg, 91% yield) as a semi-solid. This material was used without further purification. ¹H NMR (400 MHz, CDCl₃): δ 4.36 (br s, 1H), 3.03-2.92 (m, 1H), 2.59-2.50 (m, 1H), 2.16-2.04 (m, 2H), 1.78-1.66 (m, 3H), 1.43 (s, 9H), 1.32 (s, 3H), 1.30-1.19 (m, 4H), 1.06 (d, J=6.3 Hz, 6H), one proton merged with solvent peaks.

### Step 2. tert-Butyl ((1s,4s)-4-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-1-methylcyclohexyl)carbamate

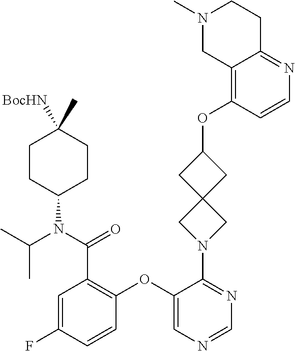 [SMILES: CC(C)N(C(=O)c1cc(F)ccc1Oc1cncnc1N1CC2(CC(Oc3ccnc4c3CN(C)CC4)C2)C1)[C@H]1CC[C@@](C)(NC(=O)OC(C)(C)C)CC1]

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 4 equivalents of tert-butyl ((1s,4s)-4-(isopropylamino)-1-methylcyclohexyl)carbamate. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain tert-butyl ((1s,4s)-4-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-1-methylcyclohexyl)carbamate as a solid.

Yield: 60.8%; LCMS (Method A): Rt=2.15 min, m/z=744.5 [M+H]⁺, 96.00%.

### Step 3. N-((1s,4s)-4-Amino-4-methylcyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl

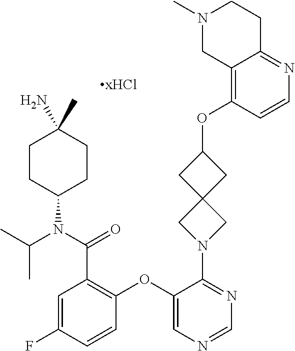 [SMILES: CC(C)N(C(=O)c1cc(F)ccc1Oc1cncnc1N1CC2(CC(Oc3ccnc4c3CN(C)CC4)C2)C1)[C@H]1CC[C@@](C)(N)CC1]

This compound was synthesized following the procedure described for the synthesis of Example 20, step 3, starting with 1 equivalent of tert-butyl ((1s,4s)-4-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-1-methylcyclohexyl)carbamate. After completion, the reaction was concentrated under reduced pressure. The residue was triturated with EtOAc, and the supernatant was decanted. The solid was dried under vacuum to obtain crude N-((1s,4s)-4-amino-4-methylcyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl as a solid. This material was used without further purification.

Yield: 90%; LCMS (Method A): Rt=1.75 min, m/z=644.4 [M+H]⁺, 84.96%.

### Step 4. N-((1s,4s)-4-(Dimethylamino)-4-methylcyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

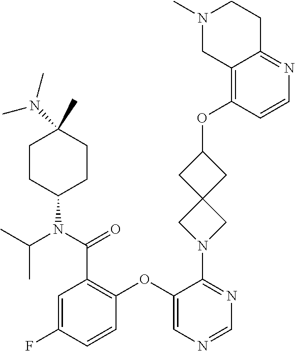 [SMILES: CC(C)N(C(=O)c1cc(F)ccc1Oc1cncnc1N1CC2(CC(Oc3ccnc4c3CN(C)CC4)C2)C1)[C@H]1CC[C@@](C)(N(C)C)CC1]

This compound was synthesized following the procedure described for the synthesis of Example 20, step 4, starting with 1 equivalent of N-((1s,4s)-4-amino-4-methylcyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl, except that 3 equivalents of DIPEA, 50 equivalents of formaldehyde (37% in H₂O), 0.05 equivalent of AcOH, and 2 equivalents of STAB were used. After completion, the reaction was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN), then by Prep HPLC (Method M) to obtain N-((1s,4s)-4-(dimethylamino)-4-methylcyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 26.9%; ¹H NMR (400 MHz, DMSO-d₆): δ 8.27 (s, 1H), 8.18 (d, J=5.7 Hz, 1H), 7.74-7.68 (m, 1H), 7.29-7.19 (m, 2H), 7.12-7.07 (m, 1H), 6.68-6.64 (m, 1H), 4.79-4.70 (m, 1H), 4.33-4.10 (m, 4H), 3.48-3.41 (m, 1H), 3.37 (s, 2H), 3.26-3.17 (m, 1H), 2.85-2.72 (m, 4H), 2.66-2.60 (m, 2H), 2.36 (s, 3H), 2.30-2.20 (m, 2H), 2.13-2.04 (m, 6H), 1.94-1.63 (m, 4H), 1.51-0.84 (m, 10H), 0.79-0.63 (m, 3H); LCMS (Method H): Rt=1.39 min, m/z=672.3 [M+H]⁺; HPLC (Method C): Rt=8.86 min, 98.84%; Chiral SFC (Method AO): Rt=1.68 min, 100%.

### Example 122. N-((1r,4r)-4-(Dimethylamino)-4-methylcyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 122)

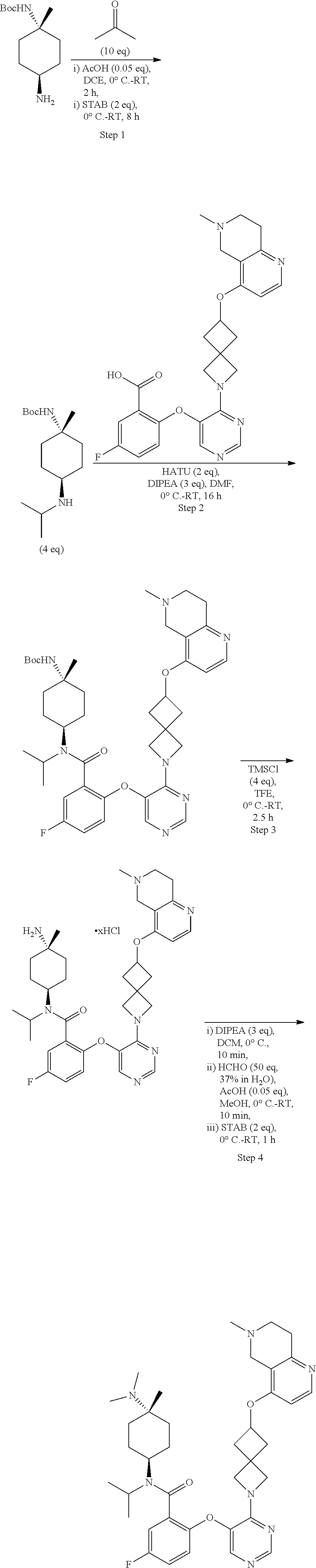

### Step 1. tert-Butyl ((1r,4r)-4-(isopropylamino)-1-methylcyclohexyl)carbamate

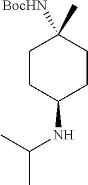 [SMILES: CC(C)N[C@H]1CC[C@](C)(NC(=O)OC(C)(C)C)CC1]

This compound was synthesized following the procedure described for the synthesis of Intermediate 1, starting with 1 equivalent of tert-butyl ((1r,4r)-4-amino-1-methylcyclohexyl)carbamate, except that 10 equivalents of acetone was used, and DCE was used as the solvent. After work-up, the filtrate was concentrated under reduced pressure to obtain crude tert-butyl ((1r,4r)-4-(isopropylamino)-1-methylcyclohexyl)carbamate as a solid. This material was used without further purification.

Yield: 97%; ¹H NMR (400 MHz, CDCl₃): δ 4.32 (br s, 1H), 3.04-2.93 (m, 1H), 2.60-2.51 (m, 1H), 2.17-2.05 (m, 2H), 1.79-1.69 (m, 2H), 1.45 (s, 9H), 1.34 (s, 3H), 1.32-1.21 (m, 4H), 1.08 (d, J=6.3 Hz, 6H), one proton merged with solvent peaks.

### Step 2. tert-Butyl ((1r,4r)-4-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-1-methylcyclohexyl)carbamate

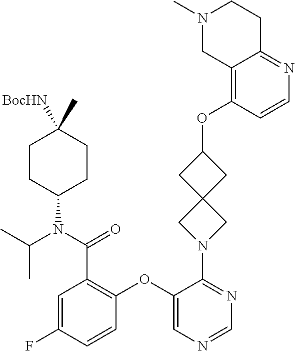 [SMILES: CC(C)N(C(=O)c1cc(F)ccc1Oc1cncnc1N1CC2(CC(Oc3ccnc4c3CN(C)CC4)C2)C1)[C@H]1CC[C@@](C)(NC(=O)OC(C)(C)C)CC1]

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.55 equivalents of tert-butyl ((1r,4r)-4-(isopropylamino)-1-methylcyclohexyl)carbamate, except that 2 equivalents of HATU was used. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain tert-butyl ((1r,4r)-4-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-1-methylcyclohexyl)carbamate as a solid.

Yield: 22.48%; LCMS (Method A): Rt=2.14 min, m/z=744.2 [M+H]⁺, 91.60%.

### Step 3. N-((1r,4r)-4-Amino-4-methylcyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl

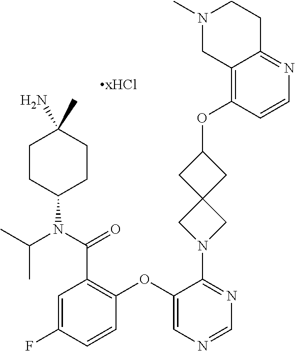 [SMILES: CC(C)N(C(=O)c1cc(F)ccc1Oc1cncnc1N1CC2(CC(Oc3ccnc4c3CN(C)CC4)C2)C1)[C@H]1CC[C@@](C)(N)CC1]

This compound was synthesized following the procedure described for the synthesis of Example 20, step 3, starting with 1 equivalent of tert-butyl ((1r,4r)-4-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-1-methylcyclohexyl)carbamate. After completion, the reaction was concentrated under reduced pressure. The residue was triturated with EtOAc, and the supernatant was decanted. The solid was dried under vacuum to obtain crude N-((1r,4r)-4-amino-4-methylcyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl as a solid. This material was used without further purification.

Yield: 94%; LCMS (Method A): Rt=1.70 min, m/z=644.4 [M+H]⁺, 94.82%.

### Step 4. N-((1r,4r)-4-(Dimethylamino)-4-methylcyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

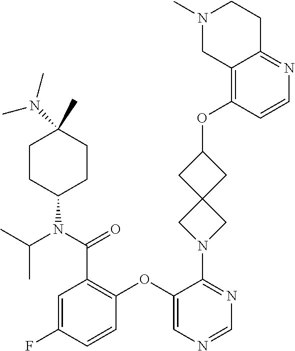 [SMILES: CC(C)N(C(=O)c1cc(F)ccc1Oc1cncnc1N1CC2(CC(Oc3ccnc4c3CN(C)CC4)C2)C1)[C@H]1CC[C@@](C)(N(C)C)CC1]

This compound was synthesized following the procedure described for the synthesis of Example 20, step 4, starting with 1 equivalent of N-((1r,4r)-4-amino-4-methylcyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl, except that 3 equivalents of DIPEA, 50 equivalents of formaldehyde (37% in H₂O), 0.05 equivalent of AcOH, and 2 equivalents of STAB were used. After completion, the reaction was concentrated under reduced pressure. The crude was purified by reverse phase chromatography using a SiliCycle SiliaSep C18 column (12 g) on Biotage isolera one (Mobile phase A: 10 mM NH₄HCO₃ in water, B: ACN), then by Prep HPLC (Method M) to obtain N-((1r,4r)-4-(dimethylamino)-4-methylcyclohexyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 31.3%; ¹H NMR (400 MHz, DMSO-d₆): δ 8.27 (s, 1H), 8.18 (d, J=5.5 Hz, 1H), 7.73-7.68 (m, 1H), 7.29-7.19 (m, 2H), 7.13-7.06 (m, 1H), 6.69-6.63 (m, 1H), 4.80-4.71 (m, 1H), 4.33-4.10 (m, 4H), 3.52-3.41 (m, 1H), 3.37 (br s, 2H), 3.27-3.14 (m, 1H), 2.86-2.71 (m, 4H), 2.66-2.60 (m, 2H), 2.37 (s, 3H), 2.27-2.20 (m, 2H), 2.12-2.02 (m, 6H), 1.94-1.63 (m, 4H), 1.49-0.86 (m, 10H), 0.79-0.59 (m, 3H); LCMS (Method A): Rt=1.92 min, m/z=672.3 [M+H]⁺; HPLC (Method C): Rt=9.34 min, 99.43%; Chiral SFC (Method J): Rt=4.70 min, 99.53%.

### Example 123. N-((1s,3r)-3-(Dimethylamino)-3-ethylcyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 123)

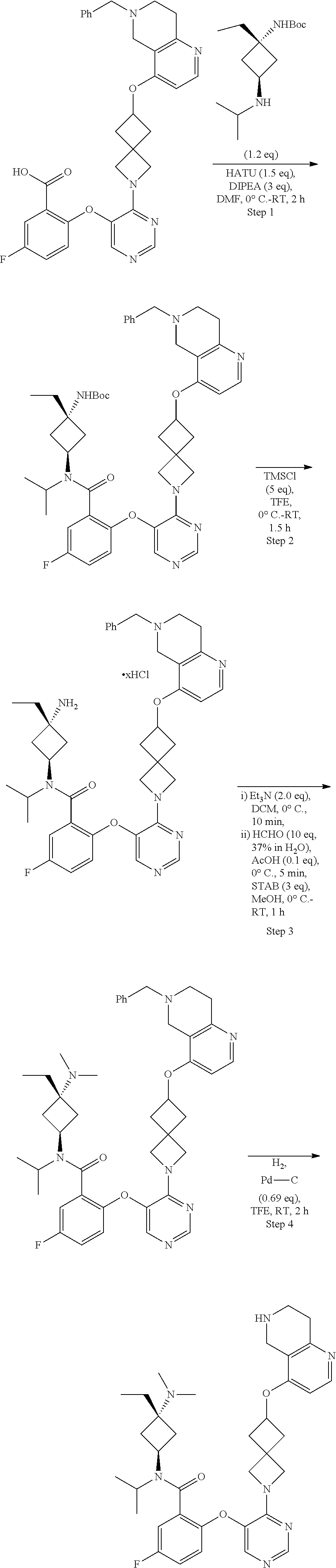

### Step 1. tert-Butyl ((1r,3s)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-1-ethylcyclobutyl)carbamate

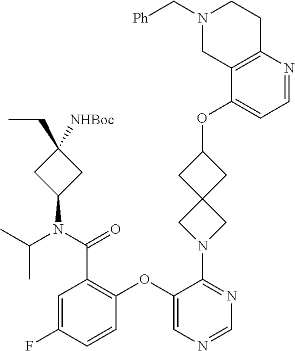 [SMILES: CC[C@]1(NC(=O)OC(C)(C)C)C[C@H](N(C(=O)c2cc(F)ccc2Oc2cncnc2N2CC3(CC(Oc4ccnc5c4CN(Cc4ccccc4)CC5)C3)C2)C(C)C)C1]

This compound was synthesized following the procedure described for the synthesis of Example 1, Step 2, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.2 equivalents of tert-butyl ((1r,3s)-1-ethyl-3-(isopropylamino)cyclobutyl)carbamate. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl ((1r,3s)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-1-ethylcyclobutyl)carbamate as a solid.

Yield: 56.3%; ¹H NMR (400 MHz, DMSO-d₆): δ 8.35-8.25 (m, 1H), 8.18 (d, J=5.6 Hz, 1H), 7.83-7.70 (m, 1H), 7.44-7.16 (m, 7H), 7.14-6.98 (m, 1H), 6.96-6.82 (m, 1H), 6.69-6.58 (m, 1H), 4.78-4.64 (m, 1H), 4.38-4.00 (m, 4H), 3.77-3.60 (m, 3H), 3.47 (s, 2H), 2.85-2.73 (m, 4H), 2.71-2.65 (m, 2H), 2.29-2.12 (m, 4H), 2.00-1.74 (m, 2H), 1.70-1.52 (m, 1H), 1.47-0.56 (m, 18H), two protons merged with solvent peaks; LCMS (Method A): Rt=2.39 min, m/z=806.3 [M+H]⁺, 86.79%.

### Step 2. N-((1s,3r)-3-Amino-3-ethylcyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl

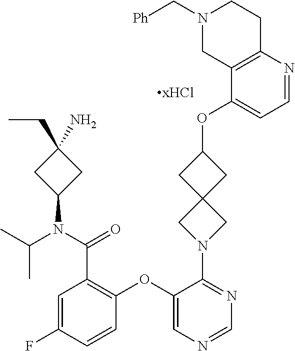 [SMILES: CC[C@]1(N)C[C@H](N(C(=O)c2cc(F)ccc2Oc2cncnc2N2CC3(CC(Oc4ccnc5c4CN(Cc4ccccc4)CC5)C3)C2)C(C)C)C1]

This compound was synthesized following the procedure described for the synthesis of Example 20, Step 3, starting with 1 equivalent of tert-butyl ((1r,3s)-3-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-1-ethylcyclobutyl)carbamate, except that 5 equivalents of TMSCl was used. After completion, the reaction was concentrated under reduced pressure and the residue was triturated with EtOAc. The organic layer was decanted and the solid was dried under vacuum to obtain crude N-((1s,3r)-3-amino-3-ethylcyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl as a solid. This material was used without further purification. Quantitative yield; LCMS (Method A): Rt=1.97 min, m/z=706.2 [M+H]⁺, 85.40%.

### Step 3. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-N-((1s,3r)-3-(dimethylamino)-3-ethylcyclobutyl)-5-fluoro-N-isopropylbenzamide

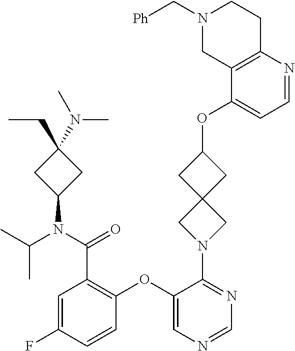 [SMILES: CC[C@]1(N(C)C)C[C@H](N(C(=O)c2cc(F)ccc2Oc2cncnc2N2CC3(CC(Oc4ccnc5c4CN(Cc4ccccc4)CC5)C3)C2)C(C)C)C1]

This compound was synthesized following the procedure described for the synthesis of Example 20, Step 4, starting with 1 equivalent of N-((1s,3r)-3-amino-3-ethylcyclobutyl)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl, except that 0.1 equivalent of AcOH, and 3 equivalents of STAB were used. After work-up, the crude was purified by reverse phase chromatography using a SiliCycle SiliaSep C18 column (80 g) on Biotage isolera one (Mobile phase A: 10 mM NH₄HCO₃ in water, B: ACN), then by achiral Prep SFC (Method C) to obtain 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1s,3r)-3-(dimethylamino)-3-ethylcyclobutyl)-5-fluoro-N-isopropylbenzamide as a solid.

Yield: 47.7%; ¹H NMR (400 MHz, DMSO-d₆): δ 8.31-8.25 (m, 1H), 8.19 (d, J=5.4 Hz, 1H), 7.86-7.54 (m, 1H), 7.41-7.02 (m, 8H), 6.69-6.61 (m, 1H), 4.78-4.67 (m, 1H), 4.42-4.06 (m, 4H), 4.02-3.73 (m, 1H), 3.70-3.59 (m, 3H), 3.46 (s, 2H), 2.90-2.65 (m, 8H), 2.27-2.15 (m, 5H), 2.06-1.98 (m, 3H), 1.88-1.59 (m, 2H), 1.51-0.73 (m, 9H), two protons merged with solvent peaks; LCMS (Method A): Rt=2.26 min, m/z=734.4 [M+H]⁺, 95.45%.

### Step 4. N-((1s,3r)-3-(Dimethylamino)-3-ethylcyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

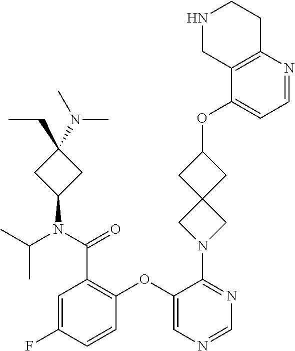 [SMILES: CC[C@]1(N(C)C)C[C@H](N(C(=O)c2cc(F)ccc2Oc2cncnc2N2CC3(CC(Oc4ccnc5c4CNCC5)C3)C2)C(C)C)C1]

This compound was synthesized following the procedure described for the synthesis of Example 20, step 5, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-N-((1s,3r)-3-(dimethylamino)-3-ethylcyclobutyl)-5-fluoro-N-isopropylbenzamide, except that 0.69 equivalent of Pd-C was used. After filtration and concentration, the crude was purified by reverse phase chromatography using a SiliCycle SiliaSep C18 column (40 g) on Biotage isolera one (Mobile phase A: 10 mM NH₄HCO₃ in water, B: ACN) to obtain N-((1s,3r)-3-(dimethylamino)-3-ethylcyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 46.3%; ¹H NMR (400 MHz, DMSO-d₆): δ 8.31-8.24 (m, 1H), 8.16 (d, J=5.5 Hz, 1H), 7.83-7.54 (m, 1H), 7.35-6.96 (m, 3H), 6.69-6.56 (m, 1H), 4.84-4.65 (m, 1H), 4.36-4.09 (m, 4H), 4.06-3.75 (m, 1H), 3.73-3.59 (m, 3H), 2.94 (t, J=5.8 Hz, 2H), 2.89-2.72 (m, 3H), 2.68 (br t, J=5.5 Hz, 2H), 2.28-2.21 (m, 2H), 2.19 (s, 3H), 2.14-2.06 (m, 1H), 2.03 (s, 3H), 1.88-1.60 (m, 3H), 1.50-0.75 (m, 9H), two protons merged with solvent peaks; LCMS (Method A): Rt=1.67 min, m/z=644.2 [M+H]⁺; HPLC (Method C): Rt=8.42 min, 99.67%; Chiral SFC (Method AAE): Rt=0.92 min, 100%.

### Example 124. N-((1s,3r)-3-(Dimethylamino)-3-ethylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 124)

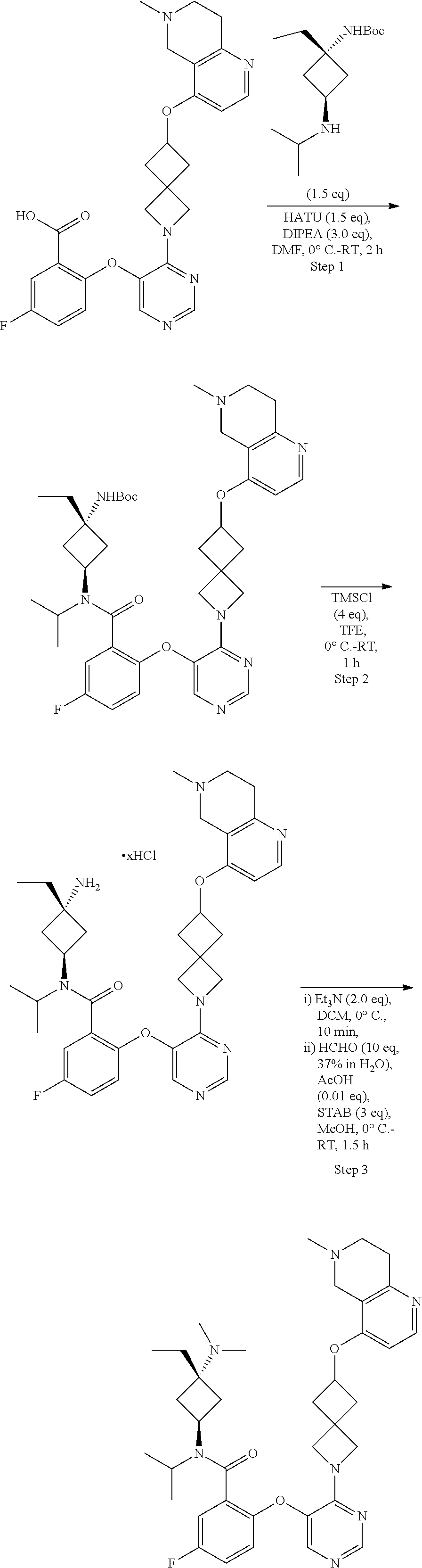

### Step 1. tert-Butyl ((1r,3s)-1-ethyl-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclobutyl)carbamate

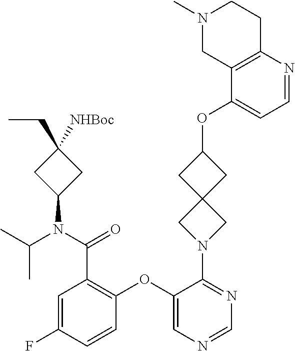 [SMILES: CC[C@]1(NC(=O)OC(C)(C)C)C[C@H](N(C(=O)c2cc(F)ccc2Oc2cncnc2N2CC3(CC(Oc4ccnc5c4CN(C)CC5)C3)C2)C(C)C)C1]

This compound was synthesized following the procedure described for the synthesis of Example 1, Step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.5 equivalents of tert-butyl (1-ethyl-3-(isopropyl amino)cyclobutyl)carbamate. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain tert-butyl ((1r,3s)-1-ethyl-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclobutyl)carbamate as a solid.

Yield: 74.3%; ¹H NMR (400 MHz, DMSO-d₆): δ 8.35-8.25 (m, 1H), 8.18 (d, J=5.5 Hz, 1H), 7.80-7.70 (m, 1H), 7.34-7.15 (m, 2H), 7.13-6.98 (m, 1H), 6.95-6.81 (m, 1H), 6.71-6.60 (m, 1H), 4.81-4.70 (m, 1H), 4.38-4.03 (m, 4H), 3.99-3.63 (m, 2H), 3.36 (s, 2H), 2.88-2.73 (m, 4H), 2.66-2.60 (m, 2H), 2.36 (s, 3H), 2.31-2.17 (m, 4H), 2.02-1.72 (m, 3H), 1.70-1.59 (m, 1H), 1.48-0.60 (m, 18H); LCMS (Method A): Rt=2.06 min, m/z=730.3 [M+H]⁺, 95.14%.

### Step 2. N-((1s,3r)-3-Amino-3-ethylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl

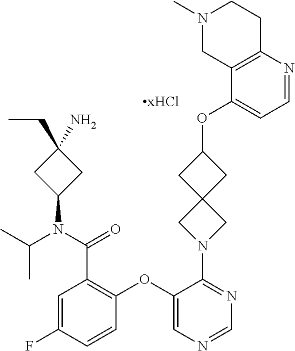 [SMILES: CC[C@]1(N)C[C@H](N(C(=O)c2cc(F)ccc2Oc2cncnc2N2CC3(CC(Oc4ccnc5c4CN(C)CC5)C3)C2)C(C)C)C1]

This compound was synthesized following the procedure described for the synthesis of Example 20, Step 3, starting with 1 equivalent of tert-butyl ((1r,3s)-1-ethyl-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)cyclobutyl)carbamate. After completion, the reaction was concentrated under reduced pressure and the residue was triturated with EtOAc. The organic layer was decanted and the solid was dried under vacuum to obtain crude N-((1s,3r)-3-amino-3-ethylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl as a solid. This material was used without further purification. Quantitative yield; LCMS (Method H): Rt=1.00 min, m/z=630.3 [M+H]⁺, 98.44%.

### Step 3. N-((1s,3r)-3-(Dimethylamino)-3-ethylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

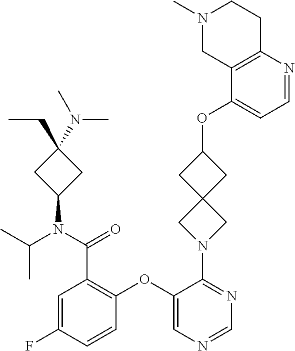 [SMILES: CC[C@]1(N(C)C)C[C@H](N(C(=O)c2cc(F)ccc2Oc2cncnc2N2CC3(CC(Oc4ccnc5c4CN(C)CC5)C3)C2)C(C)C)C1]

This compound was synthesized following the procedure described for the synthesis of Example 20, step 4, starting with 1 equivalent of N-((1s,3r)-3-amino-3-ethylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl, except that 0.01 equivalent of AcOH, and 3 equivalents of STAB were used. After work-up, the crude product was purified by reverse phase chromatography using a SiliCycle SiliaSep C18 column (120 g) on Biotage isolera one (Mobile phase A: 10 mM NH₄HCO₃ in water, B: ACN) to obtain N-((1s,3r)-3-(dimethylamino)-3-ethylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 38.2%; ¹H NMR (400 MHz, DMSO-d₆): δ 8.34-8.24 (m, 1H), 8.18 (d, J=5.4 Hz, 1H), 7.83-7.54 (m, 1H), 7.35-7.00 (m, 3H), 6.71-6.59 (m, 1H), 4.75 (quin, J=6.3 Hz, 1H), 4.44-4.08 (m, 4H), 4.06-3.58 (m, 2H), 3.36 (br s, 2H), 2.91-2.72 (m, 4H), 2.66-2.60 (m, 2H), 2.36 (s, 3H), 2.31-2.22 (m, 2H), 2.18 (s, 3H), 2.15-2.05 (m, 1H), 2.02 (s, 3H), 1.89-1.58 (m, 3H), 1.50-0.76 (m, 9H), two protons merged with solvent peaks; LCMS (Method A): Rt=1.82 min, m/z=658.3 [M+H]⁺; HPLC (Method C): Rt=9.24 min, 96.79%; Chiral SFC (Method J): Rt=4.70 min, 98.53%.

### Example 125. N-(4-(Dimethylamino)bicyclo[2.1.1]hexan-1-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 125)

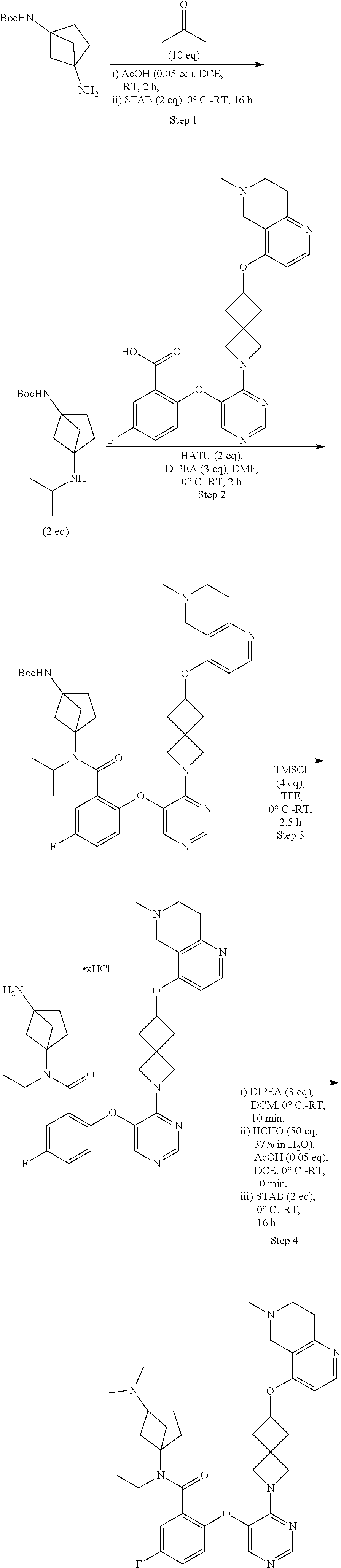

### Step 1. tert-Butyl (4-(isopropylamino)bicyclo[2.1.1]hexan-1-yl)carbamate

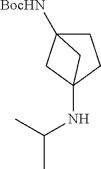 [SMILES: CC(C)NC12CCC(NC(=O)OC(C)(C)C)(C1)C2]

This compound was synthesized following the procedure described for the synthesis of 5 Intermediate 1, starting with 1 equivalent of tert-butyl (4-aminobicyclo[2. 1.1]hexan-1-yl)carbamate, except that 10 equivalents of acetone was used, and DCE was used as the solvent. After work-up, the filtrate was concentrated under reduced pressure to obtain crude tert-butyl (4-(isopropylamino)bicyclo[2.1.1]hexan-1-yl)carbamate as a semi-solid. This material was used without further purification.

Yield: 56.10/. ¹H NMR (400 MHz, DMSO-d₆): δ 7.15 (br s, 1H), 2.88-2.91 (m, 1H), 1.90-1.80 (m, 1H), 1.73-1.61 (m, 4H), 1.52-1.46 (m, 2H), 1.41 (br s, 2H), 1.37 (s, 9H), 0.96 (d, J=6.3 Hz, 6H); LCMS (Method A): Rt=1.68 min m/z=255.1 [M+H]⁺; 78.23%.

### Step 2. tert-Butyl (4-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)bicyclo[2.1.1]hexan-1-yl)carbamate

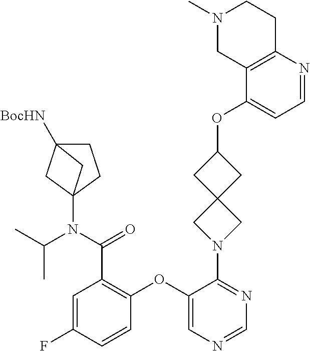 [SMILES: CC(C)N(C(=O)c1cc(F)ccc1Oc1cncnc1N1CC2(CC(Oc3ccnc4c3CN(C)CC4)C2)C1)C12CCC(NC(=O)OC(C)(C)C)(C1)C2]

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 2 equivalents of tert-butyl (4-(isopropylamino)bicyclo[2.1.1]hexan-1-yl)carbamate, except that 2 equivalents of HATU was used. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl (4-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)bicyclo[2.1.1]hexan-1-yl)carbamate as a solid.

Yield: 26.4%; LCMS (Method A): Rt=1.98 min, m/z=728.2 [M+H]⁺, 98.15%.

### Step 3. N-(4-Aminobicyclo[2.1.1]hexan-1-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl

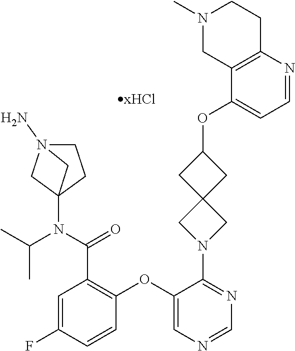

This compound was synthesized following the procedure described for the synthesis of Example 20, step 3, starting with 1 equivalent of tert-butyl (4-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)bicyclo[2.1.1]hexan-1-yl)carbamate. After completion, the reaction was concentrated under reduced pressure. The residue was triturated with EtOAc, and the supernatant was decanted. The solid was dried under vacuum to obtain crude N-(4-aminobicyclo[2.1.1]hexan-1-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl as a solid. This material was used without further purification.

Quantitative yield; LCMS (Method A): Rt=1.67 min, m/z=628.4 [M+H]⁺, 96.11%.

### Step 4. N-(4-(Dimethylamino)bicyclo[2.1.1]hexan-1-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

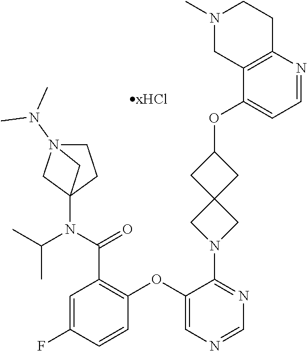

This compound was synthesized following the procedure described for the synthesis of Example 20, step 4, starting with 1 equivalent of N-(4-aminobicyclo[2.1.1]hexan-1-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl, except that 3 equivalents of DIPEA, 50 equivalents of formaldehyde (37% in H₂O), 0.05 equivalent of AcOH, and 2 equivalents of STAB were used. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: CAN) to obtain N-(4-(dimethylamino)bicyclo[2.1.1]hexan-1-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 31.5%; IH NMR (400 MHz, DMSO-d₆): δ 8.28 (s, 1H), 8.19 (d, J=5.5 Hz, 1H), 7.75-7.64 (m, 1H), 7.32-7.19 (m, 2H), 7.09-7.01 (m, 1H), 6.65 (br d, J=5.5 Hz, 1H), 4.80-4.68 (m, 1H), 4.36-4.09 (m, 4H), 3.90-3.51 (m, 1H), 3.37 (s, 2H), 2.86-2.78 (m, 4H), 2.63 (br t, J=5.8 Hz, 2H), 2.37 (s, 3H), 2.30-2.20 (m, 2H), 2.19-2.02 (m, 6H), 1.95-1.62 (m, 3H), 1.59-1.07 (m, 10H), one proton merged with solvent peaks; LCMS (Method A): Rt=1.74 min, m/z=656.3 [M+H]⁺; HPLC (Method A): Rt=5.02 min, 99.33%.

### Example 126. 5-Fluoro-N-isopropyl-2-((4-(6-((6-methyl-6,7-dihydro-5H-pyrrolo[3,4-b]pyridin-4-yl)oxy)-2-azaspiro[3.3]heptan-2-yl)pyrimidin-5-yl)oxy)-N-((2s,4r)-5-methyl-5-azaspiro[3.4]octan-2-yl)benzamide (Compound No. 126)

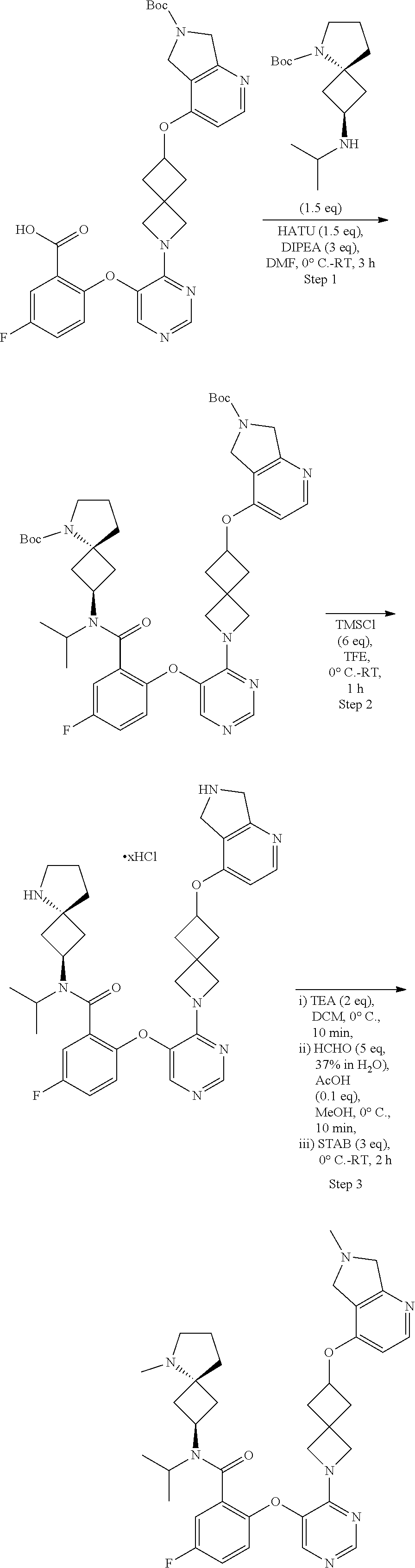 [SMILES: CC(C)(C)OC(=O)N1Cc2nccc(OC3CC4(C3)CN(c3ncncc3Oc3ccc(F)cc3C(=O)O)C4)c2C1 ; CC(C)N[C@H]1C[C@@]2(CCCN2C(=O)OC(C)(C)C)C1 ; CC(C)N(C(=O)c1cc(F)ccc1Oc1cncnc1N1CC2(CC(Oc3ccnc4c3CN(C(=O)OC(C)(C)C)C4)C2)C1)[C@H]1C[C@@]2(CCCN2C(=O)OC(C)(C)C)C1 ; CC(C)N(C(=O)c1cc(F)ccc1Oc1cncnc1N1CC2(CC(Oc3ccnc4c3CNC4)C2)C1)[C@H]1C[C@@]2(CCCN2)C1 ; CC(C)N(C(=O)c1cc(F)ccc1Oc1cncnc1N1CC2(CC(Oc3ccnc4c3CN(C)C4)C2)C1)[C@H]1C[C@@]2(CCCN2C)C1]

### Step 1. tert-Butyl 4-((2-(5-(2-(((2s,4r)-5-(tert-butoxycarbonyl)-5-azaspiro[3.4]octan-2-yl)(isopropyl)carbamoyl)-4-fluorophenoxy)pyrimidin-4-yl)-2-azaspiro[3.3]heptan-6-yl)oxy)-5,7-dihydro-6H-pyrrolo[3,4-b]pyridine-6-carboxylate

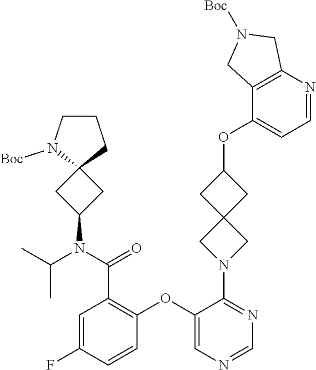 [SMILES: CC(C)N(C(=O)c1cc(F)ccc1Oc1cncnc1N1CC2(CC(Oc3ccnc4c3CN(C(=O)OC(C)(C)C)C4)C2)C1)[C@H]1C[C@@]2(CCCN2C(=O)OC(C)(C)C)C1]

This compound was synthesized following the procedure described for the synthesis of Example 1, step 2, starting with 1 equivalent of 2-((4-(6-((6-(tert-butoxycarbonyl)-6,7-dihydro-5H-pyrrolo[3,4-b]pyridin-4-yl)oxy)-2-azaspiro[3.3]heptan-2-yl)pyrimidin-5-yl)oxy)-5-fluorobenzoic acid and 1.5 equivalents of tert-butyl(2s,4r)-2-(isopropylamino)-5-azaspiro[3.4]octane-5-carboxylate. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl 4-((2-(5-(2-(((2s,4r)-5-(tert-butoxycarbonyl)-5-azaspiro[3.4]octan-2-yl)(isopropyl)carbamoyl)-4-fluorophenoxy)pyrimidin-4-yl)-2-azaspiro[3.3]heptan-6-yl)oxy)-5,7-dihydro-6H-pyrrolo[3,4-b]pyridine-6-carboxylate as a gummy liquid.

Yield: 77%; ¹H NMR (400 MHz, DMSO-d₆): δ 8.31 (d, J=5.6 Hz, 1H), 8.28 (s, 1H), 7.79 (s, 1H), 7.36-7.02 (m, 3H), 6.79 (br d, J=5.5 Hz, 1H), 4.96-4.77 (m, 1H), 4.55-4.46 (m, 4H), 4.37-4.03 (m, 5H), 3.81-3.64 (m, 1H), 3.28-3.16 (m, 3H), 3.09-2.98 (m, 1H), 2.86-2.72 (m, 3H), 2.33-2.21 (m, 4H), 2.18-2.12 (m, 1H), 1.62-1.44 (m, 18H), 1.34-0.86 (m, 8H); LCMS (Method A): Rt=2.43 min, m/z=814.4 [M+H]⁺, 98.25%.

### Step 2. 2-((4-(6-((6,7-Dihydro-5H-pyrrolo[3,4-b]pyridin-4-yl)oxy)-2-azaspiro[3.3]heptan-2-yl)pyrimidin-5-yl)oxy)-5-fluoro-N-isopropyl-N-((2s,4r)-5-azaspiro[3.4]octan-2-yl)benzamide xHCl

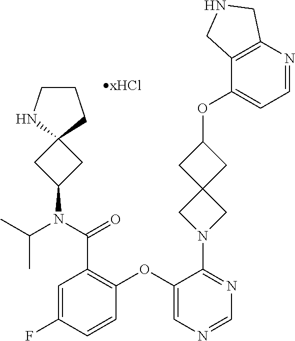 [SMILES: CC(C)N(C(=O)c1cc(F)ccc1Oc1cncnc1N1CC2(CC(Oc3ccnc4c3CNC4)C2)C1)[C@H]1C[C@@]2(CCCN2)C1]

This compound was synthesized following the procedure described for the synthesis of Example 20, Step 3, starting with 1 equivalent of tert-butyl 4-((2-(5-(2-(((2s,4r)-5-(tert-butoxycarbonyl)-5-azaspiro[3.4]octan-2-yl)(isopropyl)carbamoyl)-4-fluorophenoxy)pyrimidin-4-yl)-2-azaspiro[3.3]heptan-6-yl)oxy)-5,7-dihydro-6H-pyrrolo[3,4-b]pyridine-6-carboxylate, except that 6 equivalents of TMSCl was used. After completion, the reaction was concentrated under reduced pressure and the residue was triturated with EtOAc. The organic layer was decanted and the solid was dried under vacuum to obtain crude 2-((4-(6-((6,7-dihydro-5H-pyrrolo[3,4-b]pyridin-4-yl)oxy)-2-azaspiro[3.3]heptan-2-yl)pyrimidin-5-yl)oxy)-5-fluoro-N-isopropyl-N-((2s,4r)-5-azaspiro[3.4]octan-2-yl)benzamide xHCl as a solid. This material was used without further purification.

Yield: 94%; LCMS (Method A): Rt=1.46 min, m/z=614.2 [M+H]⁺, 93.35%.

### Step 3. 5-Fluoro-N-isopropyl-2-((4-(6-((6-methyl-6,7-dihydro-5H-pyrrolo[3,4-b]pyridin-4-yl)oxy)-2-azaspiro[3.3]heptan-2-yl)pyrimidin-5-yl)oxy)-N-((2s,4r)-5-methyl-5-azaspiro[3.4]octan-2-yl)benzamide

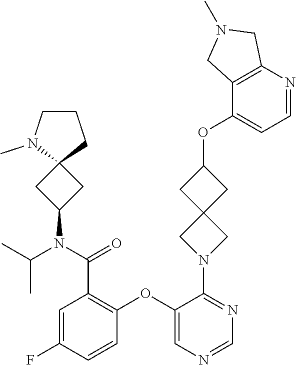 [SMILES: CC(C)N(C(=O)c1cc(F)ccc1Oc1cncnc1N1CC2(CC(Oc3ccnc4c3CN(C)C4)C2)C1)[C@H]1C[C@@]2(CCCN2C)C1]

The reaction was performed following the same procedure in two batches.

This compound was synthesized following the procedure described for the synthesis of Example 20, step 4, starting with 1 equivalent of 2-((4-(6-((6,7-dihydro-5H-pyrrolo[3,4-b]pyridin-4-yl)oxy)-2-azaspiro[3.3]heptan-2-yl)pyrimidin-5-yl)oxy)-5-fluoro-N-isopropyl-N-((2s,4r)-5-azaspiro[3.4]octan-2-yl)benzamide xHCl, except that 25 equivalents of formaldehyde (37% in H₂O), 0.1 equivalent of AcOH, and 3 equivalents of STAB were used. After work-up, the crude from the two batches were combined and purified by reverse phase chromatography using a SiliCycle SiliaSep C18 column (80 g) on Biotage isolera one (Mobile phase A: 10 mM NH₄HCO₃ in water, B: ACN) to afford 5-fluoro-N-isopropyl-2-((4-(6-((6-methyl-6,7-dihydro-5H-pyrrolo[3,4-b]pyridin-4-yl)oxy)-2-azaspiro[3.3]heptan-2-yl)pyrimidin-5-yl)oxy)-N-((2s,4r)-5-methyl-5-azaspiro[3.4]octan-2-yl)benzamide as a solid.

Yield: 24.77%; ¹H NMR (400 MHz, DMSO-d₆): δ 8.33-8.23 (m, 1H), 8.19 (br d, J=5.6 Hz, 1H), 7.80-7.55 (m, 1H), 7.36-7.04 (m, 3H), 6.74-6.65 (m, 1H), 4.78 (quin, J=6.5 Hz, 1H), 4.42-4.06 (m, 4H), 3.78 (s, 4H), 3.75-3.63 (m, 2H), 2.99-2.90 (m, 1H), 2.84-2.70 (m, 3H), 2.47 (s, 3H), 2.45-2.37 (m, 1H), 2.33 (s, 2H), 2.30-2.19 (m, 3H), 2.10-1.96 (m, 2H), 1.94-1.86 (m, 1H), 1.78-1.68 (m, 1H), 1.60-1.48 (m, 2H), 1.47-0.90 (m, 6H), one proton merged with solvent peaks; LCMS (Method H): Rt=1.16 min, m/z=642.3 [M+H]⁺; HPLC (Method G): Rt=6.47 min, 99.33%; Chiral SFC (Method K): Rt=2.51 min, 100%.

### Example 127. 5-Fluoro-N-isopropyl-N-((2s,4r)-5-methyl-5-azaspiro[3.4]octan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 127)

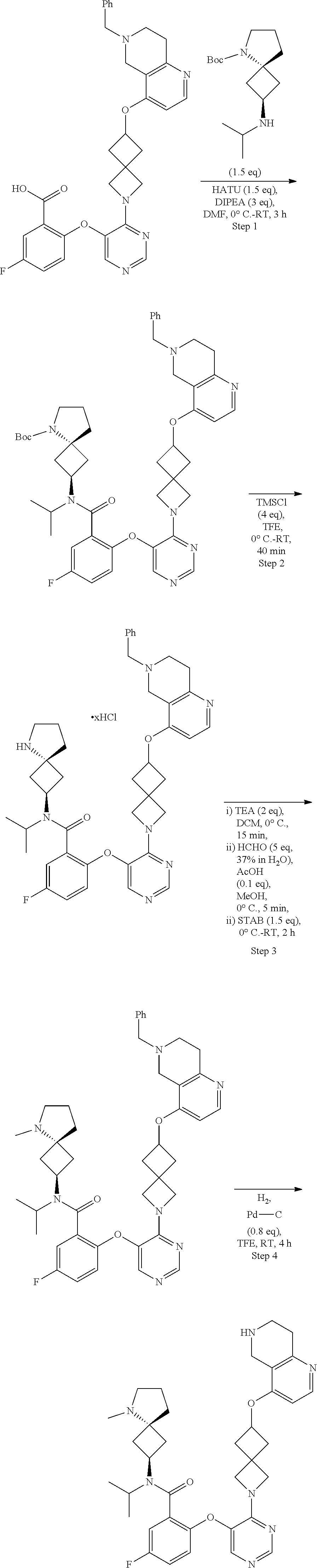

### Step 1. tert-Butyl(2s,4r)-2-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.4]octane-5-carboxylate

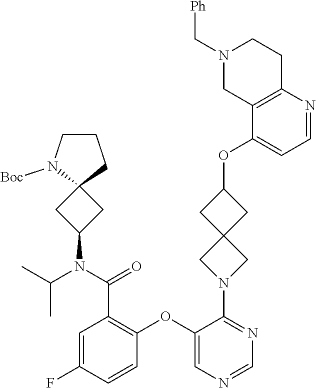 [SMILES: CC(C)N(C(=O)c1cc(F)ccc1Oc1cncnc1N1CC2(CC(Oc3ccnc4c3CN(Cc3ccccc3)CC4)C2)C1)[C@H]1C[C@@]2(CCCN2C(=O)OC(C)(C)C)C1]

This compound was synthesized following the procedure described for the synthesis of Example 1, step 2, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-yl)l)oxy)-5-fluorobenzoic acid and 1.5 equivalents of tert-butyl(2s,4r)-2-(isopropylamino)-5-azaspiro[3.4]octane-5-carboxylate. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl(2s,4r)-2-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.4]octane-5-carboxylate as a solid.

Yield: 57.9%; LCMS (Method A): Rt=2.51 min, m/z=818.3 [M+H]⁺, 94.78%.

### Step 2. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-azaspiro[3.4]octan-2-yl)benzamide xHCl

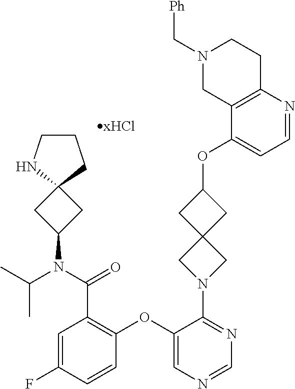 [SMILES: CC(C)N(C(=O)c1cc(F)ccc1Oc1cncnc1N1CC2(CC(Oc3ccnc4c3CN(Cc3ccccc3)CC4)C2)C1)[C@H]1C[C@@]2(CCCN2)C1]

This compound was synthesized following the procedure described for the synthesis of Example 20, Step 3, starting with 1 equivalent of tert-butyl(2s,4r)-2-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.4]octane-5-carboxylate. After completion, the reaction was concentrated under reduced pressure and the residue was triturated with EtOAc. The organic layer was decanted and the solid was dried under vacuum to obtain crude 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-azaspiro[3.4]octan-2-yl)benzamide xHCl as a solid. This material was used without further purification.

Quantitative yield; LCMS (Method H): Rt=1.25 min, m/z=718.3 [M+H]⁺, 99.18%.

### Step 3. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide

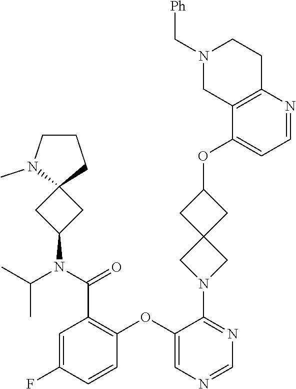 [SMILES: CC(C)N(C(=O)c1cc(F)ccc1Oc1cncnc1N1CC2(CC(Oc3ccnc4c3CN(Cc3ccccc3)CC4)C2)C1)[C@H]1C[C@@]2(CCCN2C)C1]

The reaction was performed following the same procedure in two batches.

This compound was synthesized following the procedure described for the synthesis of Example 20, step 4, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-azaspiro[3.4]octan-2-yl)benzamide xHCl, except that 5 equivalents of formaldehyde (37% in H₂O), 0.1 equivalent of AcOH, and 1.5 equivalents of STAB were used. After work-up, the crude from both batches were combined and purified by reverse phase chromatography using a SiliCycle SiliaSep C18 column (80 g) on Biotage isolera one (Mobile phase A: 10 mM NH₄HCO₃ in water, B: ACN) to obtain 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide as a solid. Combined yield: 72.1%; ¹H NMR (400 MHz, DMSO-d₆): δ 8.31-8.23 (m, 1H), 8.19 (br d, J=5.4 Hz, 1H), 7.81-7.58 (m, 1H), 7.40-7.05 (m, 8H), 6.71-6.59 (m, 1H), 4.79-4.68 (m, 1H), 4.43-4.07 (m, 4H), 3.99-3.73 (m, 1H), 3.71-3.61 (m, 3H), 3.47 (s, 2H), 3.05-2.92 (m, 1H), 2.85-2.72 (m, 4H), 2.69 (br t, J=5.1 Hz, 2H), 2.45-2.35 (m, 2H), 2.28-2.16 (m, 3H), 2.12-1.86 (m, 3H), 1.78-1.65 (m, 1H), 1.63-1.49 (m, 2H), 1.46-0.84 (m, 8H), one proton merged with solvent peaks; LCMS (Method H): Rt=1.28 min, m/z=732.3 [M+H]⁺, 97.60%.

### Step 4. 5-Fluoro-N-isopropyl-N-((2s,4r)-5-methyl-5-azaspiro[3.4]octan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

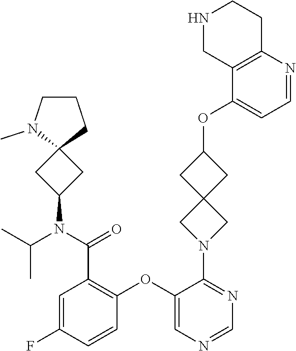 [SMILES: CC(C)N(C(=O)c1cc(F)ccc1Oc1cncnc1N1CC2(CC(Oc3ccnc4c3CNCC4)C2)C1)[C@H]1C[C@@]2(CCCN2C)C1]

This compound was synthesized following the procedure described for the synthesis of Example 20, step 5, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide, except that 0.8 equivalent of Pd-C was used. After filtration and concentration, the crude was purified by achiral Prep SFC (Method B) to obtain 5-fluoro-N-isopropyl-N-((2s,4r)-5-methyl-5-azaspiro[3.4]octan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 26.4%; ¹H NMR (400 MHz, DMSO-d;): S 8.32-8.23 (m, 1H), 8.16 (br d, J=5.1 Hz, 1H), 7.81-7.55 (m, 1H), 7.34-7.04 (m, 3H), 6.68-6.60 (m, 1H), 4.73 (quin, J=6.6 Hz, 1H), 4.41-4.05 (m, 4H), 3.80-3.62 (m, 4H), 2.96 (br t, J=5.7 Hz, 2H), 2.85-2.73 (m, 2H), 2.71-2.64 (m, 3H), 2.47-2.42 (m, 1H), 2.33 (s, 3H), 2.29-2.19 (m, 3H), 2.09-1.96 (m, 2H), 1.94-1.84 (m, 1H), 1.78-1.68 (m, 1H), 1.58-1.49 (m, 2H), 1.48-0.87 (m, 6H), one proton merged with solvent peaks; LCMS (Method H): Rt=1.05 min, m/z=642.4 [M+H]⁺; HPLC (Method G): Rt=6.19 min, 99.87%; Chiral SFC (Method J): Rt=4.71 min, 100%.

### Example 128. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((4r,6s)-1-methyl-1-azaspiro[3.3]heptan-6-yl)benzamide (Compound No. 128)

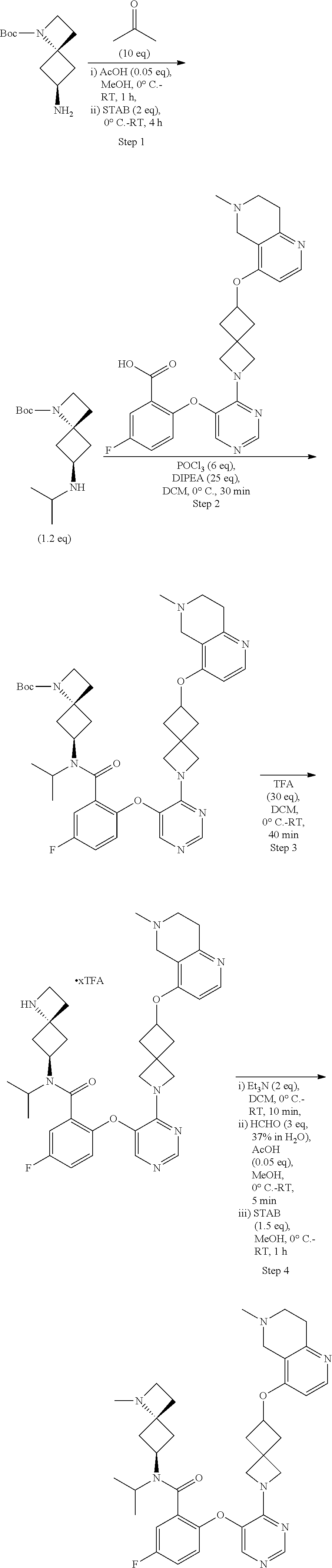

### Step 1. tert-Butyl (4r,6s)-6-(isopropylamino)-1-azaspiro[3.3]heptane-1-carboxylate

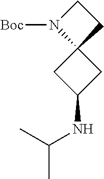 [SMILES: CC(C)N[C@H]1C[C@@]2(CCN2C(=O)OC(C)(C)C)C1]

This compound was synthesized following the procedure described for the synthesis of Example 20, Step 1, starting with 1 equivalent of tert-butyl (4r,6s)-6-amino-1-azaspiro[3.3]heptane-1-carboxylate, except that 10 equivalents of acetone, 0.05 equivalent of AcOH, and 2 equivalents of SATB were used. After completion, the reaction was quenched with water and the mixture was extracted with EtOAc. The combined organic layer was washed with brine solution, dried over anhydrous sodium sulfate, and filtered. The filtrate was concentrated under reduced pressure to obtain crude tert-butyl (4r,6s)-6-(isopropylamino)-1-azaspiro[3.3]heptane-1-carboxylate as a liquid. This material was used without further purification.

Yield: 59.3%; ¹H NMR (400 MHz, DMSO-d₆): δ 3.73-3.55 (m, 2H), 2.77-2.60 (m, 3H), 2.13 (t, J=7.4 Hz, 2H), 1.97-1.87 (m, 2H), 1.48-1.35 (m, 9H), 0.92 (d, J=6.3 Hz, 6H), two protons merged with solvent peaks; LCMS (Method A): Rt=1.50 min, m/z=255.1 [M+H]⁺, 79.28%.

### Step 2. tert-Butyl (4r,6s)-6-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5yl)oxy)benzamido)-1-azaspiro[3.3]heptane-1-carboxylate

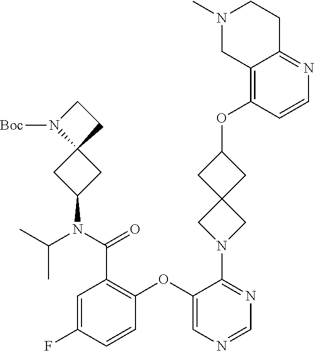 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCN2C(=O)OC(C)(C)C)C1]

This compound was synthesized following the procedure described for the synthesis of Example 20, Step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid, except that 1.2 equivalents of tert-butyl (4r,6s)-6-(isopropylamino)-1-azaspiro[3.3]heptane-1-carboxylate and 25 equivalents of DIPEA were used. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain tert-butyl (4r,6s)-6-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-1-azaspiro[3.3]heptane-1-carboxylate as a solid.

Yield: 68.5%; LCMS (Method A): Rt=2.0 min, m/z=728.2 [M+H]⁺, 99.47%.

### Step 3. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((4r,6s)-1-azaspiro[3.3]heptan-6-yl)benzamide 2,2,2-.xTFA

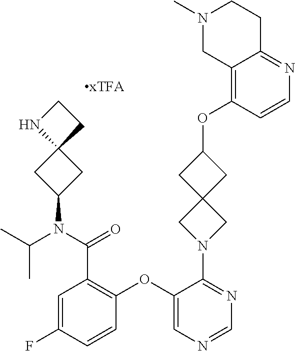 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCN2)C1]

To a stirred solution of tert-butyl (4r,6s)-6-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-1-azaspiro[3.3]heptane-1-carboxylate (200 mg, 0.275 mmol) in DCM (8 mL), trifluoroacetic acid (0.631 mL, 8.24 mmol) was added at 0° C. The reaction was stirred at RT for 40 min. The reaction progress was monitored by TLC (10% MeOH in DCM). After completion, the reaction was concentrated under reduced pressure and the residue was triturated with MTBE (50 mL). The organic layer was decanted and the solid was dried under vacuum to obtain crude 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((4r,6s)-1-azaspiro[3.3]heptan-6-yl)benzamide xTFA as a solid. This material was used without further purification.

Yield: 67.3%; LCMS (Method C): Rt=1.23-1.34 min, m/z=628.3 [M+H]⁺, 86.85%.

### Step 4. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((4r,6s)-1-methyl-1-azaspiro[3.3]heptan-6-yl)benzamide

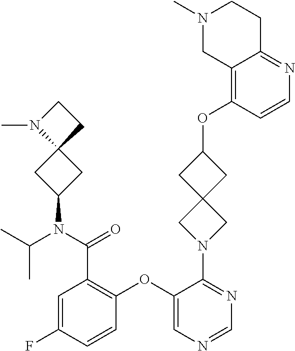 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCN2C)C1]

This compound was synthesized following the procedure described for the synthesis of Example 20, Step 4, starting with 1 equivalent of 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((4r,6s)-1-azaspiro[3.3]heptan-6-yl)benzamide xTFA, except that 3 equivalents of formaldehyde (37% in H₂O), 0.05 equivalent of AcOH, and 1.5 equivalents of STAB were used. After work-up, the crude was purified by achiral Prep SFC (Method B), then by reverse phase chromatography using a SiliCycle SiliaSep C18 column (120 g) on Biotage isolera one (Mobile phase A: 10 mM NH₄HCO₃ in water, B: ACN) to obtain impure product [Chiral SFC (Method AW): Rt=2.86 min, 62.64%]. This impure product was re-purified by Chiral Prep SFC (Method M) to afford 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((4r,6s)-1-methyl-1-azaspiro[3.3]heptan-6-yl)benzamide as solid.

Yield: 3.3%; ¹H NMR (400 MHz, DMSO-d₆): δ 8.31-8.25 (m, 1H), 8.18 (br d, J=5.5 Hz, 1H), 7.81-7.61 (m, 1H), 7.32-7.03 (m, 3H), 6.69-6.63 (m, 1H), 4.80-4.72 (m, 1H), 4.41-4.06 (m, 4H), 3.93-3.56 (m, 2H), 3.40-3.36 (m, 2H), 3.05-2.74 (m, 7H), 2.66-2.60 (m, 2H), 2.36 (s, 3H), 2.31-2.21 (m, 4H), 2.18-2.03 (m, 2H), 2.01-1.93 (m, 2H), 1.50-0.92 (m, 6H), two protons merged with solvent peaks; LCMS (Method C): Rt=1.35 min, m/z=642.2 [M+H]⁺; HPLC (Method A): Rt=4.53 min, 96.93%; Chiral SFC (Method AW): Rt=2.87 min, 100%.

### Example 129. N-((4r,6s)-1-Ethyl-1-azaspiro[3.3]heptan-6-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 129)

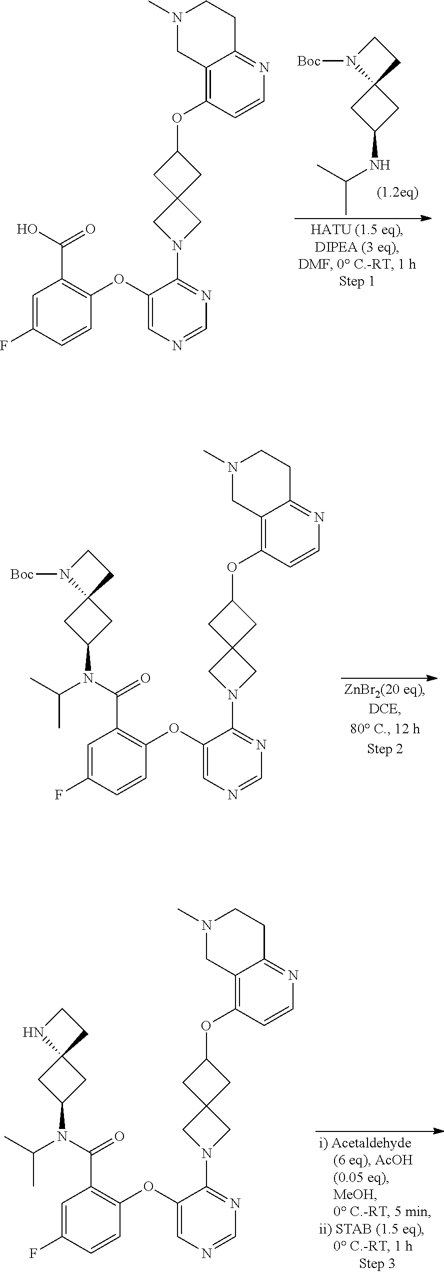 [SMILES: CN1CCc2nccc(OC3CC4(C3)CN(c3ncncc3Oc3ccc(F)cc3C(=O)O)C4)c2C1 ; CC(C)N[C@H]1C[C@@]2(CCN2C(=O)OC(C)(C)C)C1 ; CC(C)N(C(=O)c1cc(F)ccc1Oc1cncnc1N1CC2(CC(Oc3ccnc4c3CN(C)CC4)C2)C1)[C@H]1C[C@@]2(CCN2C(=O)OC(C)(C)C)C1 ; CC(C)N(C(=O)c1cc(F)ccc1Oc1cncnc1N1CC2(CC(Oc3ccnc4c3CN(C)CC4)C2)C1)[C@H]1C[C@@]2(CCN2)C1]

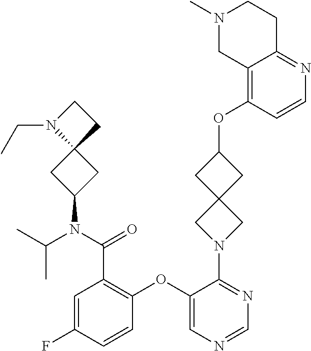 [SMILES: CCN1CC[C@]12C[C@@H](N(C(=O)c1cc(F)ccc1Oc1cncnc1N1CC3(CC(Oc4ccnc5c4CN(C)CC5)C3)C1)C(C)C)C2]

### Step 1. tert-Butyl (4r,6s)-6-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-1-azaspiro[3.3]heptan-1-carboxylate

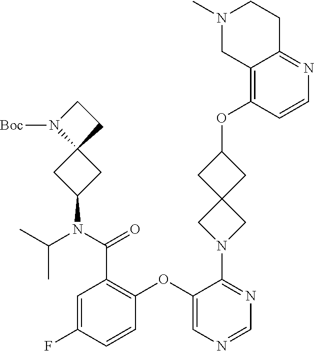 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCN2C(=O)OC(C)(C)C)C1]

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.2 equivalents of tert-butyl (4r,6s)-6-(isopropyl amino)-1-azaspiro[3.3]heptane-1-carboxylate. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl (4r,6s)-6-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-1-azaspiro[3.3]heptane-1-carboxylate as a solid.

Yield: 54.6%; LCMS (Method A): Rt=2.03 min, m/z=728.5 [M+H]⁺, 80.89%.

### Step 2. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((4r,6s)-1-azaspiro[3.3]heptan-6-yl)benzamide

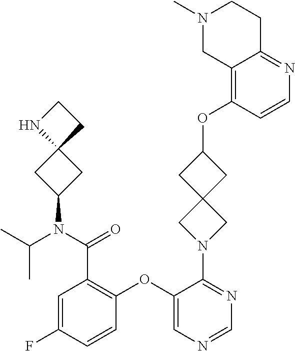 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCN2)C1]

In a 40 mL pressure vail, tert-butyl (4r,6s)-6-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-1-azaspiro[3.3]heptane-1-carboxylate (400 mg, 0.550 mmol) was dissolved in DCE (20 mL). To this solution, ZnBr₂ (2.47 g, 10.99 mmol) was added at RT and the reaction was stirred at 80° C. for 12 h. The reaction progress was monitored by LCMS (Note: The sticky solid has the product, and in the DCE layer the product was not observed). After completion, the DCE layer was decanted, and 10% aqueous NaHCO₃ solution (40 mL) was added to the sticky solid. To this mixture, 10% MeOH in DCM (50 mL) was added and the inorganic solid was filtered through a sintered funnel. The solid was washed with 10% MeOH in DCM (2×70 mL). The filtrate was taken in a separating funnel and the organic layer was separated. The organic layer was dried over anhydrous sodium sulfate and filtered, and the filtrate was concentrated under reduced pressure to afford crude 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((4r,6s)-1-azaspiro[3.3]heptan-6-yl)benzamide (300 mg, 73.6% yield) as a solid. This material was used without further purification. ¹H NMR (400 MHz, DMSO-d₆): δ 8.29 (s, 1H), 8.18 (d, J=5.6 Hz, 1H), 7.84-7.73 (m, 1H), 7.32-7.01 (m, 3H), 6.66 (d, J=5.6 Hz, 1H), 4.75 (quin, J=6.3 Hz, 1H), 4.39-4.06 (m, 5H), 4.02-3.93 (m, 1H), 3.75-3.56 (m, 1H), 3.45 (br t, J=7.3 Hz, 1H), 3.39-3.35 (m, 2H), 3.26-3.14 (m, 1H), 3.11-2.99 (m, 1H), 2.85-2.75 (m, 4H), 2.62 (br t, J=5.7 Hz, 3H), 2.36 (br s, 3H), 2.31-2.18 (m, 3H), 1.46-0.91 (m, 8H), one proton merged with solvent peaks; LCMS (Method C): Rt=1.37 min, m/z=628.4 [M+H]⁺, 84.69%.

### Step 4. N-((4r,6s)-1-Ethyl-1-azaspiro[3.3]heptan-6-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

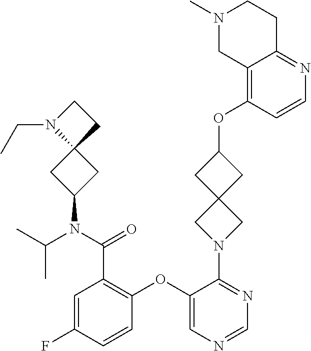 [SMILES: CCN1CC[C@]12C[C@@H](N(C(=O)c1cc(F)ccc1Oc1cncnc1N1CC3(CC(Oc4ccnc5c4CN(C)CC5)C3)C1)C(C)C)C2]

In a 25 mL round bottom flask under a nitrogen atmosphere, 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((4r,6s)-1-azaspiro[3.3]heptan-6-yl)benzamide (100 mg, 0.159 mmol) was dissolved in MeOH (5 mL). To this solution, AcOH (0.478 mg, 7.96 μmol) and acetaldehyde (42.1 mg, 0.956 mmol) were added at 0° C. The reaction was stirred for 5 min at the same temperature. STAB (50.6 mg, 0.239 mmol) was added to the reaction mixture at 0° C., and the reaction was stirred at RT for 1 h. The reaction progress was monitored by LCMS. After completion, the reaction was concentrated under reduced pressure. To this residue, 10% NaHCO₃ solution (10 mL) was added and the mixture was extracted with 10% MeOH in DCM (2×50 mL). The combined organic layer was dried over anhydrous sodium sulfate and filtered, and the filtrate was concentrated under reduced pressure. The crude was purified by Prep HPLC (Method A) to obtain N-((4r,6s)-1-ethyl-1-azaspiro[3.3]heptan-6-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (18.81 mg, 17.78% yield) as a solid. ¹H NMR (400 MHz, DMSO-d₆): δ 8.32-8.25 (m, 11H), 8.18 (br d, J=5.5 Hz, 11H), 7.83-7.59 (m, 1H), 7.32-7.02 (m, 3H), 6.70-6.60 (m, 1H), 4.81-4.70 (m, 1H), 4.41-4.08 (m, 4H), 3.98-3.72 (m, 1H), 3.70-3.55 (m, 1H), 3.36 (br s, 2H), 3.04-2.92 (m, 1H), 2.91-2.74 (m, 6H), 2.63 (br t, J=5.6 Hz, 2H), 2.36 (s, 3H), 2.31-2.17 (m, 3H), 2.09-1.91 (m, 2H), 1.52-0.68 (m, 9H), four protons merged with solvent peaks; LCMS (Method A): Rt=1.78 min, m/z=656.5 [M+H]⁺; HPLC (Method C): Rt=8.13 min, 98.95%; Chiral SFC (Method J): Rt=5.66 min, 97.09%.

### Example 130. N-((4r,6s)-1-(2,2-Difluoroethyl)-1-azaspiro[3.3]heptan-6-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 130)

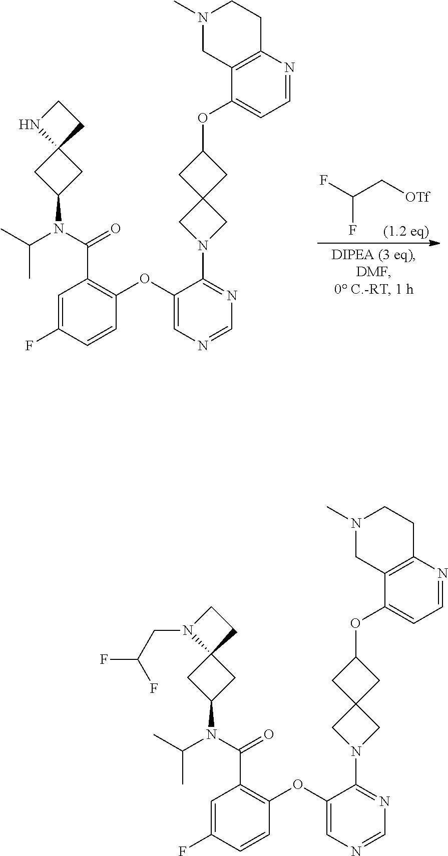 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCN2)C1 ; O=S(=O)(OCC(F)F)C(F)(F)F ; CC(C)N(C(=O)c1cc(F)ccc1Oc1cncnc1N1CC2(CC(Oc3ccnc4c3CN(C)CC4)C2)C1)[C@H]1C[C@@]2(CCN2CC(F)F)C1]

This compound was synthesized following the procedure described for the synthesis of Example 72, step 1, starting with 1 equivalent of 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((4r,6s)-1-azaspiro[3.3]heptan-6-yl)benzamide and 1.2 equivalents of 2,2-difluoroethyl trifluoromethanesulfonate, except that 3 equivalents of DIPEA was used, and DMF was used as the solvent. The reaction was stirred at RT. After completion, the reaction was quenched with 10% aqueous NaHCO₃, and the mixture was extracted with 10% MeOH in DCM. The combined organic layer was dried over anhydrous sodium sulfate and filtered, and the filtrate was concentrated under reduced pressure. The crude was purified by Prep HPLC (Method L) to obtain N-((4r,6s)-1-(2,2-difluoroethyl)-1-azaspiro[3.3]heptan-6-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 12.10%; ¹H NMR (400 MHz, DMSO-d₆): δ 8.32-8.25 (m, 1H), 8.18 (d, J=5.4 Hz, 1H), 7.81-7.66 (m, 1H), 7.36-7.03 (m, 3H), 6.72-6.59 (m, 1H), 6.20-5.66 (m, 1H), 4.75 (quin, J=6.7 Hz, 1H), 4.42-4.06 (m, 4H), 3.92-3.78 (m, 1H), 3.72-3.50 (m, 1H), 3.35 (br s, 2H), 3.13-2.88 (m, 4H), 2.84-2.74 (m, 5H), 2.62 (br t, J=5.4 Hz, 2H), 2.36 (br s, 3H), 2.31-2.21 (m, 2H), 2.19-1.99 (m, 3H), 1.51-0.90 (m, 6H), two protons merged with solvent peaks; LCMS (Method A): Rt=1.79 min, m/z=692.2 [M+H]⁺; HPLC (Method C): Rt=9.50 min, 98.80%; Chiral SFC (Method J): Rt=3.82 min, 98.33%.

### Example 131. N-((2S,3R)-1,2-Dimethylazetidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 131)

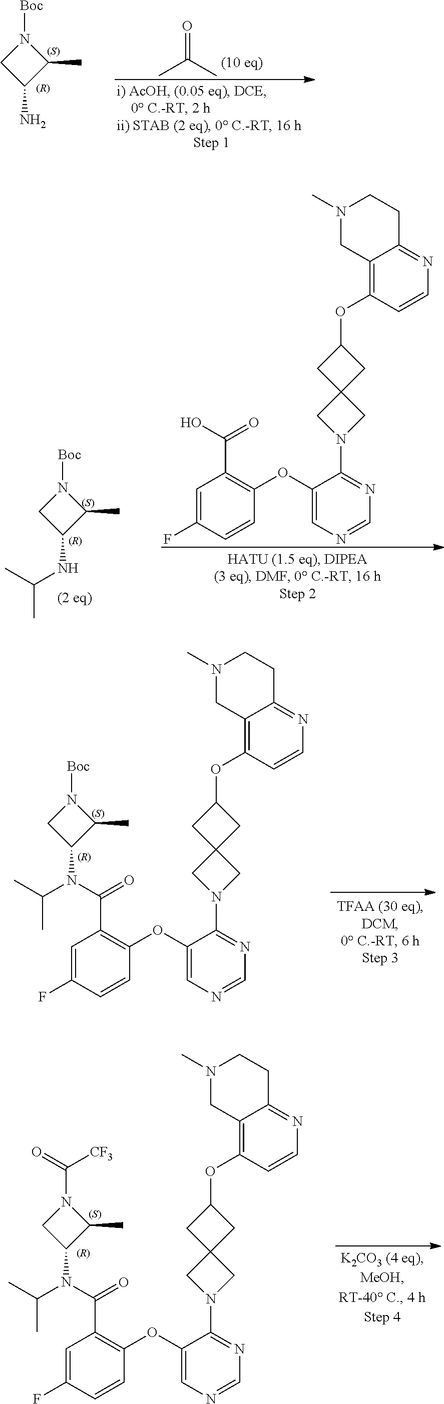

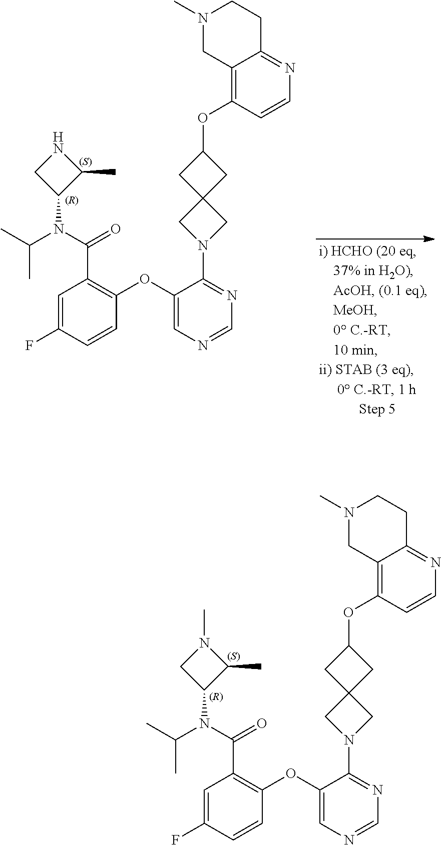

### Step 1. tert-Butyl (2S,3R)-3-(isopropylamino)-2 methylazetidine-1-carboxylate

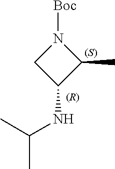

This compound was synthesized following the procedure described for the synthesis of Intermediate 1, starting with 1 equivalent of tert-butyl(2S,3R)-3-amino-2-methylazetidine-1-carboxylate, except that 10 equivalents of acetone was used, and DCE was used as the solvent. After work-up, the filtrate was concentrated under reduced pressure to obtain crude tert-butyl (2S,3R)-3-(isopropylamino)-2-methylazetidine-1-carboxylate as a semi-solid. This material was used without further purification.

Yield: 75%; ¹H NMR (400 MHz, DMSO-d₆): δ 3.88-3.73 (m, 2H), 3.39-3.33 (m, 11H), 3.07-2.99 (m, 1H), 2.75-2.66 (m, 1H), 2.27-2.15 (m, 1H), 1.38 (s, 9H), 1.28 (d, J=6.3 Hz, 3H), 0.96-0.89 (m, 6H).

### Step 2. tert-Butyl(2S,3R)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-2-methylazetidine-1-carboxylate

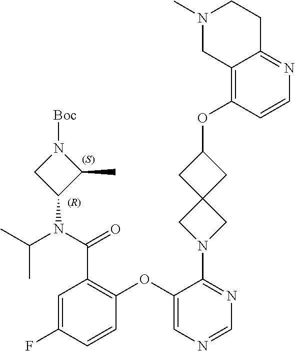

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 2 equivalents of tert-butyl(2S,3R)-3-(isopropylamino)-2-methylazetidine-1-carboxylate. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain tert-butyl(2S,3R)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-2-methylazetidine-1-carboxylate as a solid.

Yield: 51.9%; ¹H NMR (400 MHz, DMSO-d₆): δ 8.32-8.27 (m, 1H), 8.17 (d, J=5.5 Hz, 1H), 7.83-7.77 (m, 1H), 7.43-7.23 (m, 2H), 7.20-7.01 (m, 1H), 6.72-6.61 (m, 1H), 4.96-4.86 (m, 1H), 4.35-4.05 (m, 5H), 3.88-3.63 (m, 3H), 2.86-2.70 (m, 4H), 2.62 (br t, J=5.8 Hz, 2H), 2.36 (s, 3H), 2.29-2.22 (m, 2H), 1.43-1.23 (m, 12H), 1.10-0.87 (m, 6H), three protons merged with solvent peaks; LCMS (Method A): Rt=1.98 min, m/z=702.2 [M+H]⁺, 98.95%.

### Step 3. 5-Fluoro-N-isopropyl-N-((2S,3R)-2-methyl-1-(2,2,2-trifluoroacetyl)azetidin-3-yl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

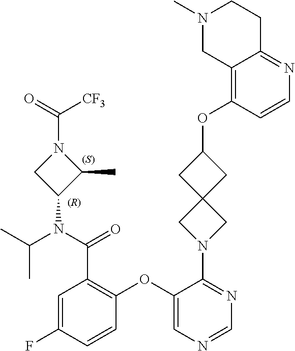

This compound was synthesized following the procedure described for the synthesis of Example 1, step 3, starting with 1 equivalent of tert-butyl(2S,3R)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-2-methylazetidine-1-carboxylate, except that 30 equivalents of trifluoroacetic anhydride was used. After completion, the reaction was concentrated under reduced pressure. The residue was re-concentrated from EtOAc to obtain crude 5-fluoro-N-isopropyl-N-((2S,3R)-2-methyl-1-(2,2,2-trifluoroacetyl)azetidin-3-yl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (0.290 g, 66.5% yield) as a solid. This material was used without further purification. LCMS (Method A): Rt=1.87 min, m/z=698.2 [M+H]⁺, 68.40%.

### Step 4. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2S,3R)-2-methylazetidin-3-yl)benzamide

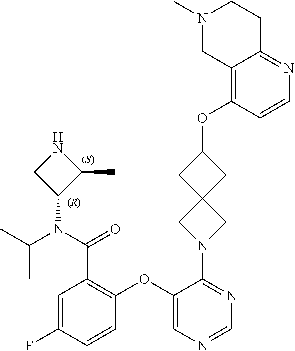

In a dried, 25 mL round bottom flask under a nitrogen atmosphere, 5-fluoro-N-isopropyl-N-((2S,3R)-2-methyl-1-(2,2,2-trifluoroacetyl)azetidin-3-yl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (280 mg, 0.401 mmol) was dissolved in MeOH (5 mL). To this solution, K₂C03 (222 mg, 1.605 mmol) was added at RT, and the reaction was stirred at 40° C. for 4 h. The reaction progress was monitored by LCMS. After completion, the reaction was diluted with water (20 mL), and the mixture was extracted with DCM (3×20 mL). The combined organic layer was dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2S,3R)-2-methylazetidin-3-yl)benzamide (210 mg, 81% yield) as a solid. LCMS (Method A): Rt=1.64 min, m/z=602.2 [M+H]⁺, 93.08%.

### Step 5. N-((2S,3R)-1,2-Dimethylazetidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

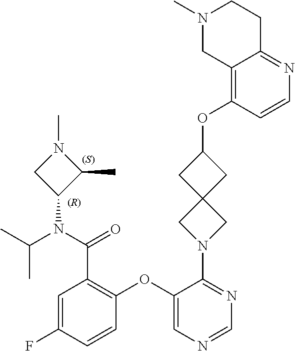

This compound was synthesized following the procedure described for the synthesis of Example 76, starting with 1 equivalent of 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2S,3R)-2-methylazetidin-3-yl)benzamide, except that 20 equivalents of formaldehyde (37% in H₂O) and 0.1 equivalent of AcOH, were used. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain N-((2S,3R)-1,2-dimethylazetidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 52.3%; ¹H NMR (400 MHz, DMSO-d₆): δ 8.31-8.24 (m, 1H), 8.18 (d, J=5.6 Hz, 1H), 7.77-7.67 (m, 1H), 7.35-7.20 (m, 2H), 7.19-7.03 (m, 1H), 6.65 (d, J=5.6 Hz, 1H), 4.79-4.70 (m, 1H), 4.34-4.01 (m, 4H), 3.98-3.85 (m, 1H), 3.81-3.57 (m, 2H), 3.46-3.40 (m, 1H), 3.36 (s, 2H), 3.30-2.99 (m, 2H), 2.85-2.75 (m, 4H), 2.63 (br t, J=5.8 Hz, 2H), 2.36 (s, 3H), 2.32-2.17 (m, 4H), 1.48-1.22 (m, 3H), 1.16-0.84 (m, 6H); LCMS (Method A): Rt=1.62 min, m/z=616.2 [M+H]⁺; HPLC (Method C): Rt=8.28 min, 97.29%; Chiral SFC (Method Y): Rt=4.67 min, 97.06%.

### Example 132. N-((2R,3R)-1,2-dimethylazetidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 132)

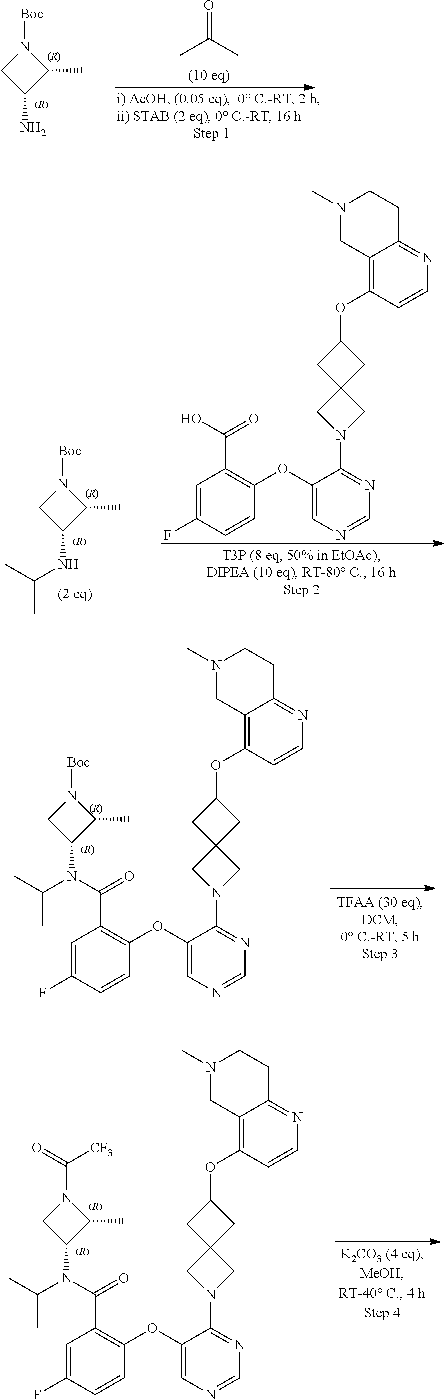

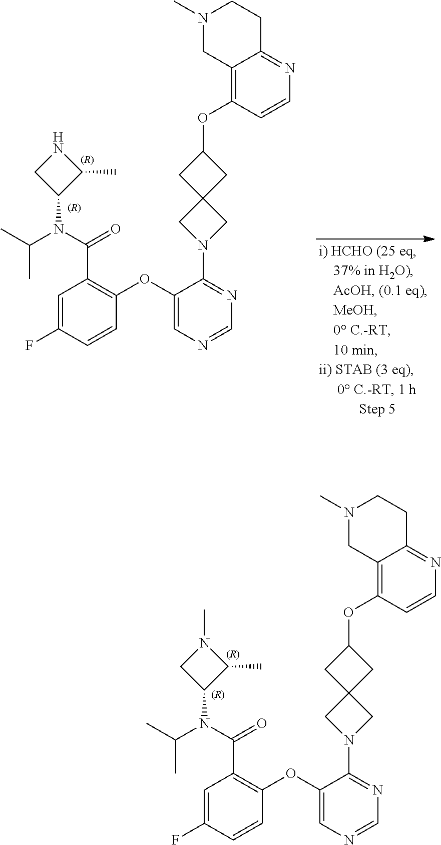

### Step 1. tert-Butyl(2R,3R)-3-(isopropylamino)-2-methylazetidine-1-carboxylate

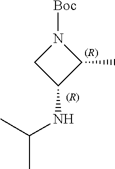

This compound was synthesized following the procedure described for the synthesis of Intermediate 1, starting with 1 equivalent of tert-butyl(2R,3R)-3-amino-2-methylazetidine-1-carboxylate, except that 10 equivalents of acetone was used, and DCE was used as the solvent. After work-up, the filtrate was concentrated under reduced pressure to obtain crude tert-butyl (2R,3R)-3-(isopropylamino)-2-methylazetidine-1-carboxylate as a semi-solid. This material was used without further purification.

Yield: 67%; ¹H NMR (400 MHz, DMSO-dh): S 4.24 (quin, J=6.8 Hz, 1H), 3.97-3.85 (m, 2H), 3.65-3.56 (m, 1H), 3.50-3.39 (m, 1H), 2.69-2.60 (m, 1H), 1.36 (s, 9H), 1.21 (d, J=6.5 Hz, 3H), 0.96-0.93 (m, 6H).

### Step 2. tert-Butyl(2R,3R)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-2-methylazetidine-1-carboxylate

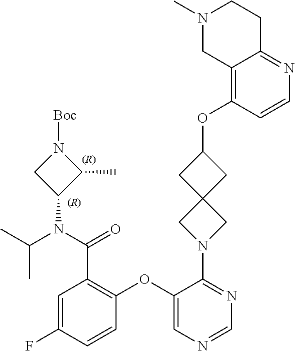

This compound was synthesized following the procedure described for the synthesis of Example 25, Step 1, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 2 equivalents of tert-butyl(2R,3R)-3-(isopropylamino)-2-methylazetidine-1-carboxylate. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl(2R,3R)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-2-methylazetidine-1-carboxylate as a solid.

Yield: 19.07%; LCMS (Method A): Rt=1.94 min, m/z=702.2 [M+H]⁺97.23%.

### Step 3. 5-Fluoro-N-isopropyl-N-((2R,3R)-2-methyl-1-(2,2,2-trifluoroacetyl)azetidin-3-yl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

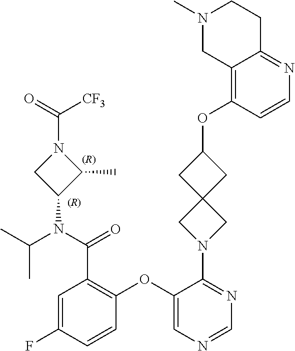

This compound was synthesized following the procedure described for the synthesis of Example 1, step 3, starting with 1 equivalent of tert-butyl(2R,3R)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-2-methylazetidine-1-carboxylate, except that 30 equivalents of trifluoroacetic anhydride was used. After completion, the reaction was concentrated under reduced pressure, and re-concentrated from EtOAc to obtain crude 5-fluoro-N-isopropyl-N-((2R,3R)-2-methyl-1-(2,2,2-trifluoroacetyl)azetidin-3-yl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a semi-solid. This material was used without further purification.

Yield: 62.8%; LCMS (Method A): Rt=1.83 min, m/z=698.4 [M+H]⁺, 65.77%.

### Step 4. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2R,3R)-2-methylazetidin-3-yl)benzamide

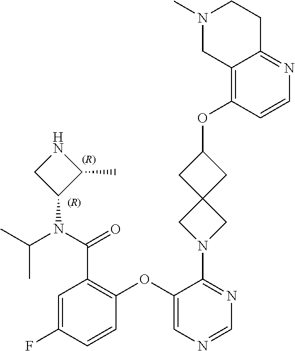

This compound was synthesized following the procedure described for the synthesis of Example 131, step 4, starting with 1 equivalent of 5-fluoro-N-isopropyl-N-((2R,3R)-2-methyl-1-(2,2,2-trifluoroacetyl)azetidin-3-yl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide. After work-up the crude was purified by reverse phase chromatography using a SiliCycle SiliaSep C18 column (40 g) on Biotage isolera one (Mobile phase A: 10 mM NH₄HCO₃ in water, B: ACN) to obtain 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2R,3R)-2-methylazetidin-3-yl)benzamide as a solid.

Yield: 54.3%; ¹H NMR (400 MHz, DMSO-d₆): δ 8.32-8.23 (m, 1H), 8.18 (d, J=5.5 Hz, 1H), 7.77-7.65 (m, 1H), 7.29-7.11 (m, 2H), 7.07-6.98 (m, 1H), 6.66 (d, J=5.5 Hz, 1H), 4.79-4.70 (m, 1H), 4.51-4.37 (m, 1H), 4.33-4.02 (m, 5H), 3.99-3.53 (m, 2H), 3.36 (s, 2H), 2.85-2.74 (m, 4H), 2.66-2.59 (m, 2H), 2.36 (s, 3H), 2.30-2.21 (m, 2H), 1.28-0.93 (m, 9H), two protons merged with the solvent peaks; LCMS (Method A): Rt=1.70 min, m/z=602.4 [M+H]⁺, 93.59%.

### Step 5. N-((2R,3R)-1,2-Dimethylazetidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

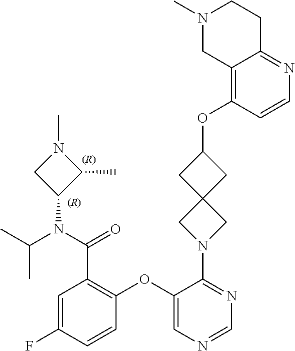

This compound was synthesized following the procedure described for the synthesis of Example 76, starting with 1 equivalent of 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2R,3R)-2-methylazetidin-3-yl)benzamide, except that 25 equivalents of formaldehyde (37% in H₂O) and 0.1 equivalent of AcOH, were used. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN), then by Prep HPLC (Method H) to obtain N-((2R,3R)-1,2-dimethylazetidin-3-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 34.3%; ¹H NMR (400 MHz, DMSO-d₆): δ 8.35-8.22 (m, 1H), 8.21-8.16 (m, 1H), 7.91-7.65 (m, 1H), 7.31-6.85 (m, 3H), 6.71-6.61 (m, 1H), 4.80-4.70 (m, 1H), 4.42-4.04 (m, 6H), 3.55-3.41 (m, 1H), 3.37 (s, 2H), 3.25-2.93 (m, 2H), 2.85-2.72 (m, 4H), 2.63 (br t, J=5.9 Hz, 2H), 2.36 (s, 3H), 2.30-2.17 (m, 2H), 2.10 (br s, 2H), 1.65-1.49 (m, 3H), 1.37-0.85 (m, 6H), one proton merged with solvent peaks; LCMS (Method C): Rt=1.33 min, m/z=616.4 [M+H]⁺; HPLC (Method C): Rt=7.56 min, 98.65%; Chiral SFC (Method Y): Rt=5.19 min, 97.44%.

### Example 133. 5-Fluoro-N-isopropyl-N-((1r,3r)-3-methyl-3-(pyrrolidin-1-yl)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 133)

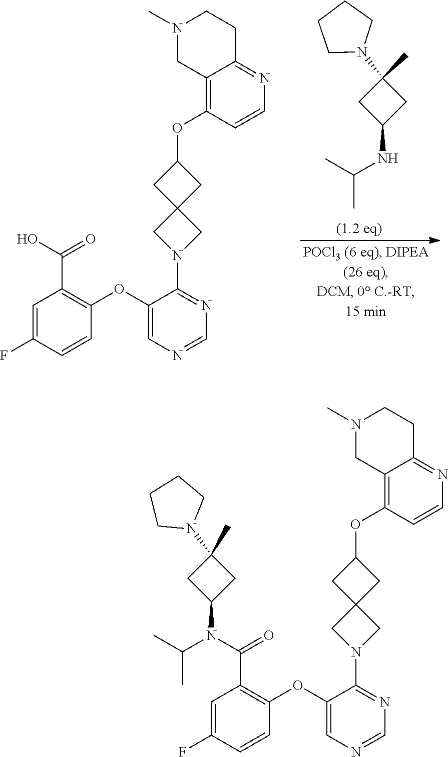 [SMILES: CN1CCc2nccc(OC3CC4(C3)CN(c3ncncc3Oc3ccc(F)cc3C(=O)O)C4)c2C1 ; CC(C)N[C@H]1C[C@](C)(N2CCCC2)C1 ; CC(C)N(C(=O)c1cc(F)ccc1Oc1cncnc1N1CC2(CC(Oc3ccnc4c3CN(C)CC4)C2)C1)[C@H]1C[C@](C)(N2CCCC2)C1]

This compound was synthesized following the procedure described for the synthesis of Example 20, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.2 equivalents of (1r,3r)—N-isopropyl-3-methyl-3-(pyrrolidin-1-yl)cyclobutan-1-amine. After work-up, the crude was purified by reverse phase chromatography using a SiliCycle SiliaSep C18 column (120 g) on Biotage isolera one (Mobile phase A: 10 mM NH₄HCO₃ in water, B: ACN), then by achiral Prep SFC (Method F) to obtain the desired product (HPLC purity: 87%). This impure product was further purified by reverse phase chromatography using a SiliCycle SiliaSep C18 column (40 g) on Biotage isolera one (Mobile phase A: 10 mM NH₄HCO₃ in water, B: ACN) to afford 5-fluoro-N-isopropyl-N-((1r,3r)-3-methyl-3-(pyrrolidin-1-yl)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 23.92%; ¹H NMR (400 MHz, DMSO-d₆): δ 8.27 (s, 1H), 8.18 (d, J=5.4 Hz, 1H), 7.82-7.53 (m, 1H), 7.34-6.99 (m, 3H), 6.69-6.58 (m, 1H), 4.74 (quin, J=6.6 Hz, 1H), 4.41-4.10 (m, 4H), 4.09-3.78 (m, 1H), 3.75-3.59 (m, 1H), 3.36 (br s, 2H), 2.87-2.75 (m, 5H), 2.65-2.59 (m, 2H), 2.36 (s, 3H), 2.31-2.05 (m, 5H), 2.01-1.75 (m, 2H), 1.72-1.55 (m, 4H), 1.48-0.89 (m, 9H), three protons merged with solvent peaks; LCMS (Method C): Rt=1.43 min, m/z=670.4 [M+H]⁺; HPLC (Method C): Rt=8.18 min, 97.74%; Chiral SFC (Method J): Rt=5.48 min, 100%.

### Example 134. N-((1r,3r)-3-(Dimethylamino)-3-methylcyclobutyl)-5-fluoro-N-isopropyl-2-((4-(6-((5,6,7,8-tetrahydro-1,7-naphthyridin-4-yl)oxy)-2-azaspiro[3.3]heptan-2-yl)pyrimidin-5-yl)oxy)benzamide (Compound No. 134)

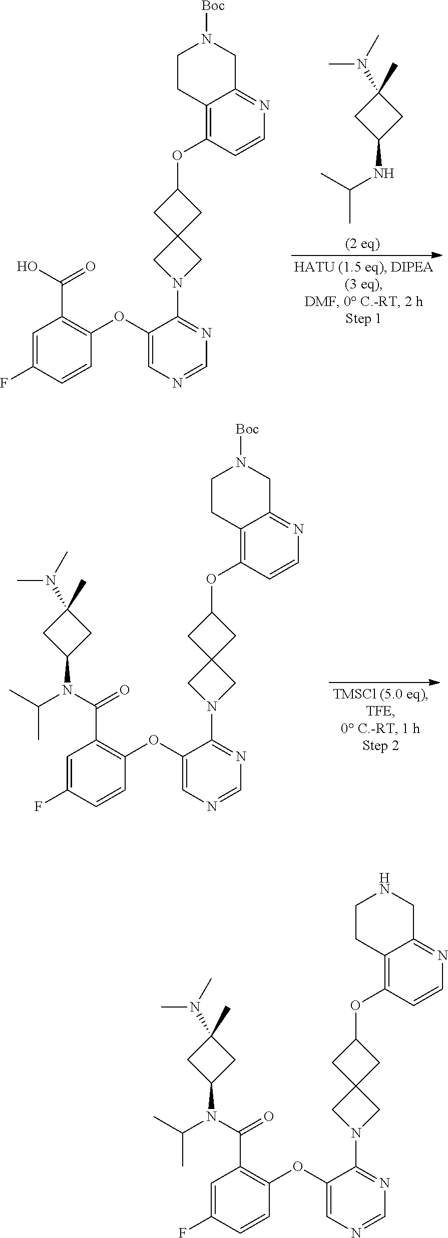 [SMILES: CC(C)(C)OC(=O)N1CCc2c(OC3CC4(C3)CN(c3ncncc3Oc3ccc(F)cc3C(=O)O)C4)ccnc2C1 ; CC(C)N[C@H]1C[C@](C)(N(C)C)C1 ; CC(C)N(C(=O)c1cc(F)ccc1Oc1cncnc1N1CC2(CC(Oc3ccnc4c3CCN(C(=O)OC(C)(C)C)C4)C2)C1)[C@H]1C[C@](C)(N(C)C)C1 ; CC(C)N(C(=O)c1cc(F)ccc1Oc1cncnc1N1CC2(CC(Oc3ccnc4c3CCNC4)C2)C1)[C@H]1C[C@](C)(N(C)C)C1]

### Step 1. tert-Butyl 4-((2-(5-(2-(((1r,3r)-3-(dimethyl amino)-3-methylcyclobutyl)(isopropyl)carbamoyl)-4-fluorophenoxy)pyrimidin-4-yl)-2-azaspiro[3.3]heptan-6-yl)oxy)-5,8-dihydro-1,7-naphthyridine-7(6H)-carboxylate Boc

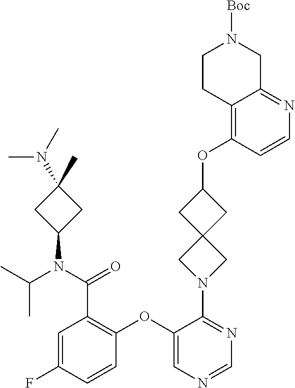 [SMILES: CC(C)N(C(=O)c1cc(F)ccc1Oc1cncnc1N1CC2(CC(Oc3ccnc4c3CCN(C(=O)OC(C)(C)C)C4)C2)C1)[C@H]1C[C@](C)(N(C)C)C1]

This compound was synthesized following the procedure described for the synthesis of Example 1, step 2, starting with l equivalent of 2-((4-(6-((7-(tert-butoxycarbonyl)-5,6,7,8-tetrahydro-1,7-naphthyridin-4-yl)oxy)-2-azaspiro[3.3]heptan-2-yl)pyrimidin-5-yl)oxy)-5-fluorobenzoic acid and 2 equivalents of (1r,3r)—N³-isopropyl-N¹,N¹,1-trimethylcyclobutane-1,3-diamine. After work-up, the crude was purified by reverse phase chromatography using a SiliCycle SiliaSep C18 column (40 g) on Biotage isolera one (Mobile phase A: 10 mM NH₄HCO₃ in water, B: ACN) to obtain tert-butyl 4-((2-(5-(2-(((1r,3r)-3-(dimethyl amino)-3-methylcyclobutyl)(isopropyl)carbamoyl)-4-fluorophenoxy)pyrimidin-4-yl)-2-azaspiro[3.3]heptan-6-yl)oxy)-5,8-dihydro-1,7-naphthyridine-7(6H)-carboxylate as a solid.

Yield: 27.3%; ¹H NMR (400 MHz, DMSO-d;): S 8.32-8.26 (m, 1H), 8.22 (br d, J=5.1 Hz, 1H), 7.81-7.57 (m, 1H), 7.31-7.03 (m, 3H), 6.76-6.66 (m, 1H), 4.77 (quin, J=6.5 Hz, 1H), 4.45 (br s, 2H), 4.37-4.09 (m, 4H), 3.85-3.65 (m, 2H), 3.63-3.53 (m, 2H), 2.87-2.75 (m, 3H), 2.61 (br t, J=5.5 Hz, 2H), 2.31-2.23 (m, 2H), 2.19-2.04 (m, 5H), 1.98 (s, 2H), 1.96-1.87 (m, 1H), 1.81-1.69 (m, 1H), 1.42 (s, 9H), 1.37-0.91 (m, 9H); LCMS (Method A): Rt=2.14 min, m/z=730.3 M+H]⁺, 94.96%.

### Step 2. N-((1r,3r)-3-(Dimethylamino))-3-methylcyclobutyl)-5-fluoro-N-isopropyl-2-((4-(6-((5,6,7,8-tetrahydro-1,7-naphthyridin-4-yl)oxy)-2-azaspiro[3.3]heptan-2-yl)pyrimidin-5-yl)oxy)benzamide

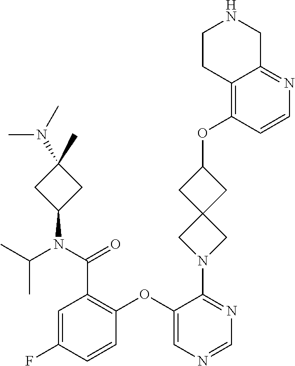 [SMILES: CC(C)N(C(=O)c1cc(F)ccc1Oc1cncnc1N1CC2(CC(Oc3ccnc4c3CCNC4)C2)C1)[C@H]1C[C@](C)(N(C)C)C1]

This compound was synthesized following the procedure described for the synthesis of Example 20, step 3, starting with 1 equivalent of tert-butyl 4-((2-(5-(2-(((1r,3r)-3-(dimethylamino)-3-methylcyclobutyl)(isopropyl)carbamoyl)-4-fluorophenoxy)pyrimidin-4-yl)-2-azaspiro[3.3]heptan-6-yl)oxy)-5,8-dihydro-1,7-naphthyridine-7(6H)-carboxylate, except that 5 equivalents of TMSCl was used. After completion, the reaction was concentrated under reduced pressure. The crude was purified by reverse phase chromatography using a SiliCycle SiliaSep C18 column (25 g) on Biotage isolera one (Mobile phase A: 10 mM NH₄HCO₃ in water, B: ACN) to obtain N-((1r,3r)-3-(dimethylamino)-3-methylcyclobutyl)-5-fluoro-N-isopropyl-2-((4-(6-((5,6,7,8-tetrahydro-1,7-naphthyridin-4-yl)oxy)-2-azaspiro[3.3]heptan-2-yl)pyrimidin-5-yl)oxy)benzamide as a solid.

Yield: 34.5%; ¹H NMR (400 MHz, DMSO-d₆): δ 8.33-8.25 (m, 1H), 8.14 (d, J=5.4 Hz, 11H), 7.80-7.58 (m, 1H), 7.34-7.04 (m, 3H), 6.68-6.60 (m, 1H), 4.73 (quin, J=6.5 Hz, 1H), 4.39-4.06 (m, 4H), 4.04-3.80 (m, 1H), 3.77 (s, 2H), 3.74-3.64 (m, 1H), 2.94-2.88 (m, 2H), 2.85-2.74 (m, 3H), 2.66-2.59 (m, 1H), 2.29-2.20 (m, 2H), 2.17-2.05 (m, 5H), 1.99 (s, 3H), 1.95-1.88 (m, 1H), 1.80-1.68 (m, 1H), 1.48-0.90 (m, 9H), one proton merged with solvent peaks; LCMS (Method H): Rt=1.15 min, m/z=630.3 [M+H]⁺; HPLC (Method G): Rt=6.31 min, 99.24%; Chiral SFC (Method H): Rt=3.90 min, 99.85%.

### Example 135. 2-((4-(6-((6,7-Dihydro-5H-pyrrolo[3,4-b]pyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide (Compound No. 135)

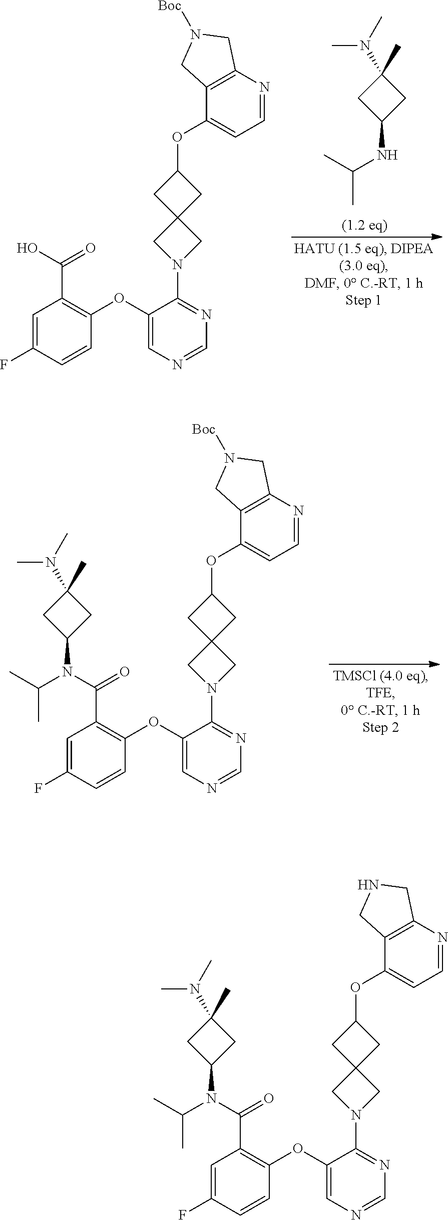 [SMILES: CC(C)(C)OC(=O)N1Cc2nccc(OC3CC4(C3)CN(c3ncncc3Oc3ccc(F)cc3C(=O)O)C4)c2C1 ; CC(C)N[C@H]1C[C@](C)(N(C)C)C1 ; CC(C)N(C(=O)c1cc(F)ccc1Oc1cncnc1N1CC2(CC(Oc3ccnc4c3CN(C(=O)OC(C)(C)C)C4)C2)C1)[C@H]1C[C@](C)(N(C)C)C1 ; CC(C)N(C(=O)c1cc(F)ccc1Oc1cncnc1N1CC2(CC(Oc3ccnc4c3CNC4)C2)C1)[C@H]1C[C@](C)(N(C)C)C1]

### Step 1. tert-Butyl 4-((2-(5-(2-(((1r,3r)-3-(dimethylamino)-3-methylcyclobutyl)(isopropyl)carbamoyl)-4-fluorophenoxy)pyrimidin-4-yl)-2-azaspiro[3.3]heptan-6-yl)oxy)-5,7-dihydro-6H-pyrrolo[3,4-b]pyridine-6-carboxylate

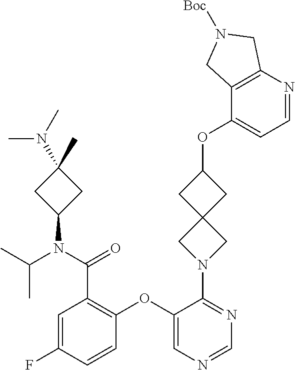 [SMILES: CC(C)N(C(=O)c1cc(F)ccc1Oc1cncnc1N1CC2(CC(Oc3ccnc4c3CN(C(=O)OC(C)(C)C)C4)C2)C1)[C@H]1C[C@](C)(N(C)C)C1]

This compound was synthesized following the procedure described for the synthesis of Example 1, step 2, starting with 1 equivalent of 2-((4-(6-((6-(tert-butoxycarbonyl)-6,7-dihydro-5H-pyrrolo[3,4-b]pyridin-4-yl)oxy)-2-azaspiro[3.3]heptan-2-yl)pyrimidin-5-yl)oxy)-5-fluorobenzoic acid and 1.2 equivalents of (1r,3r)—N³-isopropyl-N¹,N¹,1-trimethylcyclobutane-1,3-diamine were used. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl 4-((2-(5-(2-(((1r,3r)-3-(dimethylamino)-3-methylcyclobutyl)(isopropyl)carbamoyl)-4-fluorophenoxy)pyrimidin-4-yl)-2-azaspiro[3.3]heptan-6-yl)oxy)-5,7-dihydro-6H-pyrrolo[3,4-b]pyridine-6-carboxylate as a solid.

Yield: 41.3%; ¹H NMR (400 MHz, DMSO-d₆): 8.35-8.30 (m, 1H), 8.28 (d, J=5.8 Hz, 1H), 7.84-7.53 (m, 1H), 7.34-6.97 (m, 3H), 6.85-6.70 (m, 1H), 4.83 (quin, J=6.5 Hz, 1H), 4.52 (br s, 2H), 4.49 (br s, 2H), 4.36-4.08 (m, 4H), 4.05-3.77 (m, 1H), 3.74-3.59 (m, 1H), 2.88-2.74 (m, 2H), 2.32-2.24 (m, 2H), 2.19-2.09 (m, 3H), 1.99 (br s, 3H), 1.93-1.86 (m, 1H), 1.80-1.68 (m, 1H), 1.46 (s, 9H), 1.40-0.87 (m, 9H), two protons merges with solvent peaks; LCMS (Method A): Rt=2.08 min, m/z=716.3 [M+H]⁺, 95.38%.

### Step 2. 2-((4-(6-((6,7-Dihydro-5H-pyrrolo[3,4-b]pyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide

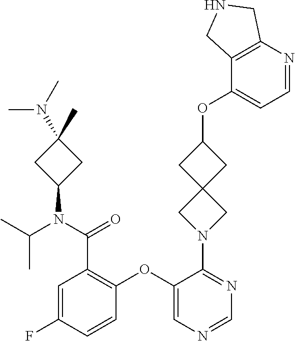 [SMILES: CC(C)N(C(=O)c1cc(F)ccc1Oc1cncnc1N1CC2(CC(Oc3ccnc4c3CNC4)C2)C1)[C@H]1C[C@](C)(N(C)C)C1]

This compound was synthesized following the procedure described for the synthesis of Example 20, step 3, starting with 1 equivalent of tert-butyl 4-((2-(5-(2-(((1r,3r)-3-(dimethylamino)-3-methylcyclobutyl)(isopropyl)carbamoyl)-4-fluorophenoxy)pyrimidin-4-yl)-2-azaspiro[3.3]heptan-6-yl)oxy)-5,7-dihydro-6H-pyrrolo[3,4-b]pyridine-6-carboxylate. After completion, the reaction was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain 2-((4-(6-((6,7-dihydro-5H-pyrrolo[3,4-b]pyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide as a solid.

Yield: 79%; ¹H NMR (400 MHz, DMSO-d₆): δ 8.32-8.25 (m, 1H), 8.21 (d, J=5.5 Hz, 1H), 7.79-7.58 (m, 1H), 7.35-7.04 (m, 3H), 6.77 (br s, 1H), 4.78 (quin, J=6.7 Hz, 1H), 4.54-4.30 (m, 1H), 4.28-4.08 (m, 3H), 4.03 (s, 2H), 3.99 (s, 2H), 3.86-3.64 (m, 2H), 2.86-2.72 (m, 2H), 2.31-2.21 (m, 2H), 2.19-1.92 (s, 6H), 1.95-1.87 (m, 1H), 1.81-1.65 (m, 1H), 1.48-0.88 (m, 9H), one proton merged with solvent peaks; LCMS (Method H): Rt=1.26 min, m/z=616.4 [M+H]⁺; HPLC (Method G): Rt=6.37 min, 99.47%; Chiral SFC (Method K): Rt=3.55 min, 100%.

### Example 136. 5-Fluoro-N-isopropyl-N-((1r,3r)-3-methyl-3-(pyrrolidin-1-yl)cyclobutyl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 136)

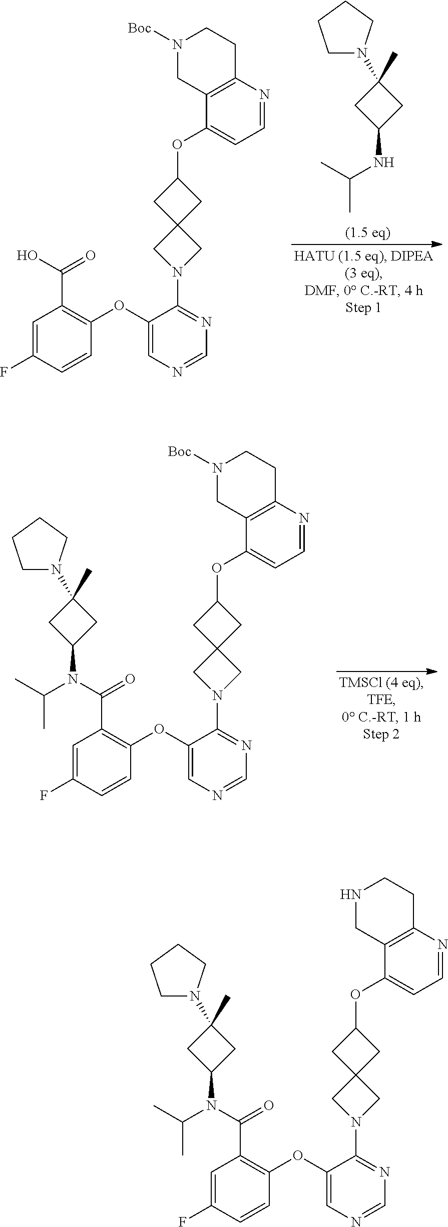 [SMILES: CC(C)(C)OC(=O)N1CCc2nccc(OC3CC4(C3)CN(c3ncncc3Oc3ccc(F)cc3C(=O)O)C4)c2C1 ; CC(C)N[C@H]1C[C@](C)(N2CCCC2)C1 ; CC(C)N(C(=O)c1cc(F)ccc1Oc1cncnc1N1CC2(CC(Oc3ccnc4c3CN(C(=O)OC(C)(C)C)CC4)C2)C1)[C@H]1C[C@](C)(N2CCCC2)C1 ; CC(C)N(C(=O)c1cc(F)ccc1Oc1cncnc1N1CC2(CC(Oc3ccnc4c3CNCC4)C2)C1)[C@H]1C[C@](C)(N2CCCC2)C1]

### Step 1. tert-Butyl 4-((2-(5S-(4-fluoro-2-(isopropyl((1r,3r)-3-methyl-3-(pyrrolidin-1-yl)cyclobutyl)carbamoyl)phenoxyl)pyrimidin-4-yl)-2-azaspiro[3.3]heptan-6-yl)oxy)-7.8-dihydro-1,6-naphthyridine-6(5H)-carboxylate

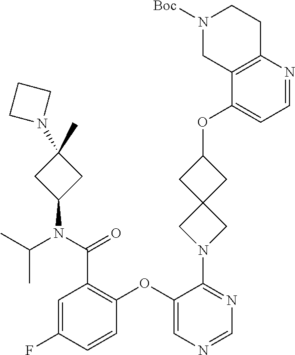 [SMILES: CC(C)N(C(=O)c1cc(F)ccc1Oc1cncnc1N1CC2(CC(Oc3ccnc4c3CN(C(=O)OC(C)(C)C)CC4)C2)C1)[C@H]1C[C@](C)(N2CCC2)C1]

This compound was synthesized following the procedure described for the synthesis of Example 1, step 2, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.5 equivalents of (1r,3r)—N-isopropyl-3-methyl-3-(pyrrolidin-1-yl)cyclobutan-1-amine. After work-up, the crude was purified by reverse phase chromatography using a SiliCycle SiliaSep C18 column (40 g) on Biotage isolera one (Mobile phase A: 10 mM NH-4HCO₃ in water, B: ACN) to obtain tert-butyl 4-((2-(5-(4-fluoro-2-(isopropyl((1r,3r)-3-methyl-3-(pyrrolidin-1-yl)cyclobutyl)carbamoyl)phenoxyl)pyrimidin-4-yl)-2-azaspiro[3.3]heptan-6-yl)oxy)-7,8-dihydro-,6-naphthyridine-6(5)-carboxylate as a solid.

Yield: 34.7%; ¹H NMR (400 MHz, DMSO-d₆): δ 8.31-8.26 (m, 1H), 8.23 (br d, J=5.5 Hz, 1H), 7.79-7.58 (m, 1H), 7.33-7.05 (m, 3H), 6.77-6.68 (m, 1H), 4.78 (quin, J=6.4 Hz, 1H), 4.37 (br s, 2H), 4.32-4.02 (m, 4H), 3.89-3.66 (m, 2H), 3.61 (br t, J=5.4 Hz, 2H), 2.90-2.74 (m, 5H), 2.41-2.35 (m, 2H), 2.31-2.06 (m, 4H), 2.02-1.74 (m, 2H), 1.71-1.53 (m, 4H), 1.43 (s, 9H), 1.34-0.87 (m, 9H), one proton merged with solvent peaks; LCMS (Method A): Rt=2.36 min, m/z=756.5 [M+H]⁺, 86.47%.

### Step 2. 5-Fluoro-N-isopropyl-N-((1r,3r)-3-methyl-3-(pyrrolidin-1-yl)cyclobutyl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

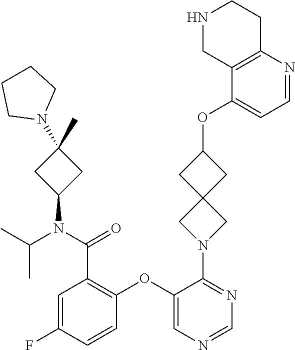 [SMILES: CC(C)N(C(=O)c1cc(F)ccc1Oc1cncnc1N1CC2(CC(Oc3ccnc4c3CNCC4)C2)C1)[C@H]1C[C@](C)(N2CCCC2)C1]

This compound was synthesized following the procedure described for the synthesis of Example 20, step 3, starting with 1 equivalent of tert-butyl 4-((2-(5-(4-fluoro-2-(isopropyl((1r,3r)-3-methyl-3-(pyrrolidin-1-yl)cyclobutyl)carbamoyl)phenoxyl)pyrimidin-4-yl)-2-azaspiro[3.3]heptan-6-yl)oxy)-7,8-dihydro-1,6-naphthyridine-6(5H)-carboxylate. After completion, the reaction concentrated under reduced pressure to obtain crude 5-fluoro-N-isopropyl-N-((1r,3r)-3-methyl-3-(pyrrolidin-1-yl)cyclobutyl)-2-((4-(6-((5,6,7,8-tetrahydro-1,6-naphthyridin-4-yl)oxy)-2-azaspiro[3.3]heptan-2-yl)pyrimidin-5-yl)oxy)benzamide xHCl. The crude was purified by reverse phase chromatography using a SiliCycle SiliaSep C18 column (40 g) on Biotage isolera one (Mobile phase A: 10 mM NH₄HCO₃ in water, B: ACN) to obtain 5-fluoro-N-isopropyl-N-((1r,3r)-3-methyl-3-(pyrrolidin-1-yl)cyclobutyl)-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 58.2%; ¹H NMR (400 MHz, DMSO-d₆): δ 8.27 (s, 1H), 8.16 (br d, J=5.3 Hz, 1H), 7.82-7.56 (m, 1H), 7.36-7.02 (m, 3H), 6.70-6.57 (m, 1H), 4.72 (quin, J=6.3 Hz, 1H), 4.39-4.01 (m, 5H), 3.90-3.72 (m, 1H), 3.69 (s, 2H), 2.95 (br t, J=5.5 Hz, 2H), 2.88-2.73 (m, 3H), 2.71-2.65 (m, 3H), 2.41-2.37 (m, 1H), 2.30-2.08 (m, 4H), 2.03-1.75 (m, 2H), 1.72-1.55 (m, 4H), 1.49-0.86 (m, 9H), two protons merged with solvent peaks; LCMS (Method H): Rt=1.24 min, m/z=656.4 [M+H]⁺; HPLC (Method C): Rt=8.11 min, 98.00%; Chiral SFC (Method E): Rt=3.86 min, 98.65%.

### Example 137. N-((1r,3r)-3-(Azetidin-1-yl)-3-methylcyclobutyl)-2-((4-(6-((6,7-dihydro-5H-pyrrolo[3,4-b]pyridin-4-yl)oxy)-2-azaspiro[3.3]heptan-2-yl)pyrimidin-5-yl)oxy)-5-fluoro-N-isopropylbenzamide (Compound No. 137)

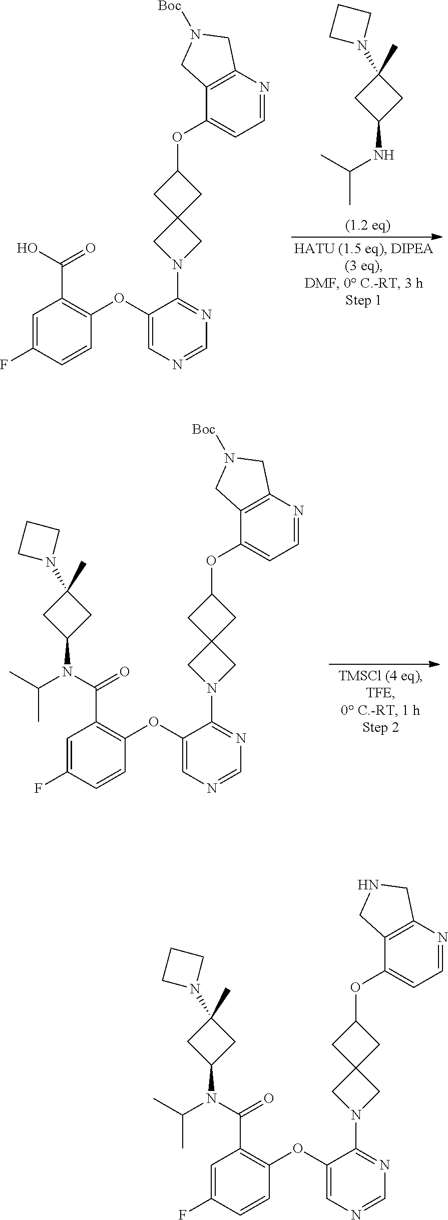 [SMILES: CC(C)(C)OC(=O)N1Cc2nccc(OC3CC4(C3)CN(c3ncncc3Oc3ccc(F)cc3C(=O)O)C4)c2C1 ; CC(C)N[C@H]1C[C@](C)(N2CCC2)C1 ; CC(C)N(C(=O)c1cc(F)ccc1Oc1cncnc1N1CC2(CC(Oc3ccnc4c3CN(C(=O)OC(C)(C)C)C4)C2)C1)[C@H]1C[C@](C)(N2CCC2)C1 ; CC(C)N(C(=O)c1cc(F)ccc1Oc1cncnc1N1CC2(CC(Oc3ccnc4c3CNC4)C2)C1)[C@H]1C[C@](C)(N2CCC2)C1]

### Step 1. tert-Butyl 4-((2-(5-(2-(((I r, 3r)-3-(azetidin-1-yl)-3-methylcyclobutyl)(isopropyl)carbamoyl)-4-fluorophenoxy)pyrimidin-4-yl)-2-azaspiro[3.3]heptan-6-yl)oxy)-5,7-dihydro-6H-pyrrolo[3,4-b]pyridine-6-carboxylate

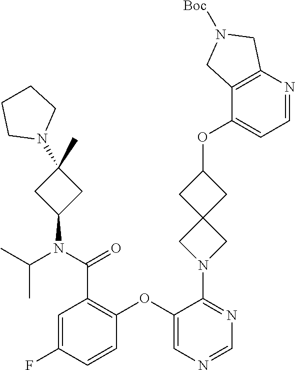 [SMILES: CC(C)N(C(=O)c1cc(F)ccc1Oc1cncnc1N1CC2(CC(Oc3ccnc4c3CN(C(=O)OC(C)(C)C)C4)C2)C1)[C@H]1C[C@](C)(N2CCCC2)C1]

This compound was synthesized following the procedure described for the synthesis of Example 1, step 2, starting with 1 equivalent of 2-((4-(6-((6-(tert-butoxycarbonyl)-6,7-dihydro-5H-pyrrolo[3,4-b]pyridin-4-yl)oxy)-2-azaspiro[3.3]heptan-2-yl)pyrimidin-5-yl)oxy)-5-fluorobenzoic acid and 1.2 equivalents of (1r,3r)-3-(azetidin-1-yl)-N-isopropyl-3-methylcyclobutan-1-amine. After work-up, the crude was purified by reverse phase chromatography using a SiliCycle SiliaSep C18 column (40 g) on Biotage isolera one (Mobile phase A: 10 mM NH₄HCO₃ in water, B: ACN) to obtain tert-butyl 4-((2-(5-(2-(((1r,3r)-3-(azetidin-1-yl)-3-methylcyclobutyl)(isopropyl)carbamoyl)-4-fluorophenoxy)pyrimidin-4-yl)-2-azaspiro[3.3]heptan-6-yl)oxy)-5,7-dihydro-6H-pyrrolo[3,4-b]pyridine-6-carboxylate as a solid.

Yield: 42.3%; ¹H NMR (400 MHz, DMSO-d₆): δ 8.39-8.30 (m, 1H), 8.29-8.25 (m, 1H), 7.80-7.61 (m, 1H), 7.36-7.04 (m, 3H), 6.84-6.75 (m, 1H), 4.83 (quin, J=6.5 Hz, 1H), 4.59-4.44 (m, 4H), 4.38-4.09 (m, 4H), 3.83-3.60 (m, 2H), 3.20-3.05 (m, 2H), 2.97-2.87 (m, 2H), 2.86-2.75 (m, 2H), 2.65-2.56 (m, 1H), 2.32-2.23 (m, 2H), 2.20-1.98 (m, 2H), 1.94-1.74 (m, 3H), 1.46 (s, 9H), 1.34-0.90 (m, 9H); LCMS (Method M): Rt=1.81 min, m/z=728.2 [M+H]⁺, 95.65%.

### Step 2. N-((1r,3r)-3-(Azetidin-1-yl)-3-methylcyclobutyl)-2-((4-(6-((6,7-dihydro-5H-pyrrolo[3,4-b]pyridin-4-yl)oxy)-2-azaspiro[3.3]heptan-2-yl)pyrimidin-5yl)oxy)-5-fluoro-N-isopropylbenzamide

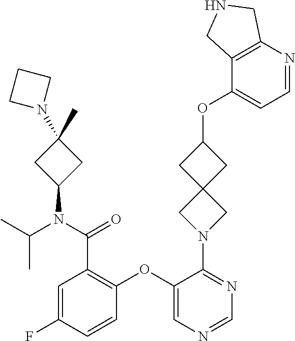 [SMILES: CC(C)N(C(=O)c1cc(F)ccc1Oc1cncnc1N1CC2(CC(Oc3ccnc4c3CNC4)C2)C1)[C@H]1C[C@](C)(N2CCC2)C1]

This compound was synthesized following the procedure described for the synthesis of Example 1, step 2, starting with 1 equivalent of tert-butyl 4-((2-(5-(2-(((1r,3r)-3-(azetidin-1-yl)-3-methylcyclobutyl)(isopropyl)carbamoyl)-4-fluorophenoxy)pyrimidin-4-yl)-2-azaspiro[3.3]heptan-6-yl)oxy)-5,7-dihydro-6H-pyrrolo[3,4-b]pyridine-6-carboxylate. After completion, the reaction was concentrated under reduced pressure to obtain crude N-((1r,3r)-3-(azetidin-1-yl)-3-methylcyclobutyl)-2-((4-(6-((6,7-dihydro-5H-pyrrolo[3,4-b]pyridin-4-yl)oxy)-2-azaspiro[3.3]heptan-2-yl)pyrimidin-5-yl)oxy)-5-fluoro-N-isopropylbenzamide xHCl. The crude was purified by reverse phase chromatography using a SiliCycle SiliaSep C18 column (40 g) on Biotage isolera one (Mobile phase A: 10 mM NH₄HCO₃ in water, B: ACN) to obtain N-((1r,3r)-3-(azetidin-1-yl)-3-methylcyclobutyl)-2-((4-(6-((6,7-dihydro-5H-pyrrolo[3,4-b]pyridin-4-yl)oxy)-2-azaspiro[3.3]heptan-2-yl)pyrimidin-5-yl)oxy)-5-fluoro-N-isopropylbenzamide as a solid.

Yield: 16.97%; ¹H NMR (400 MHz, DMSO-d₆): δ 8.29-8.25 (m, 1H), 8.21 (d, J=5.5 Hz, 1H), 7.78-7.59 (m, 1H), 7.36-7.21 (m, 2H), 7.19-7.06 (m, 1H), 6.72-6.63 (m, 1H), 4.87-4.74 (m, 1H), 4.36-4.18 (m, 3H), 4.16-4.08 (m, 1H), 4.03 (s, 2H), 3.99 (s, 2H), 3.79-3.63 (m, 2H), 3.11 (br t, J=6.6 Hz, 2H), 2.91 (br t, J=6.6 Hz, 2H), 2.85-2.72 (m, 3H), 2.31-2.21 (m, 2H), 2.19-1.98 (m, 2H), 1.92-1.70 (m, 4H), 1.50-0.90 (m, 9H); LCMS (Method H): Rt=1.19 min, m/z=628.3 [M+H]⁺; HPLC (Method G): Rt=6.41 min, 97.58%; Chiral SFC (Method AAB) Rt=1.56 min, 100%.

### Example 138. 2-((4-(6-((6,7-Dihydro-5H-pyrrolo[3,4-b]pyridin-4-yl)oxy)-2-azaspiro 13.31 heptan-2-yl)pyrimidin-5-yl)oxy)-5-fluoro-N-isopropyl-N-((1r,3r)-3-methyl-3-(pyrrolidin-1-yl)cyclobutyl)benzamide (Compound No. 138)

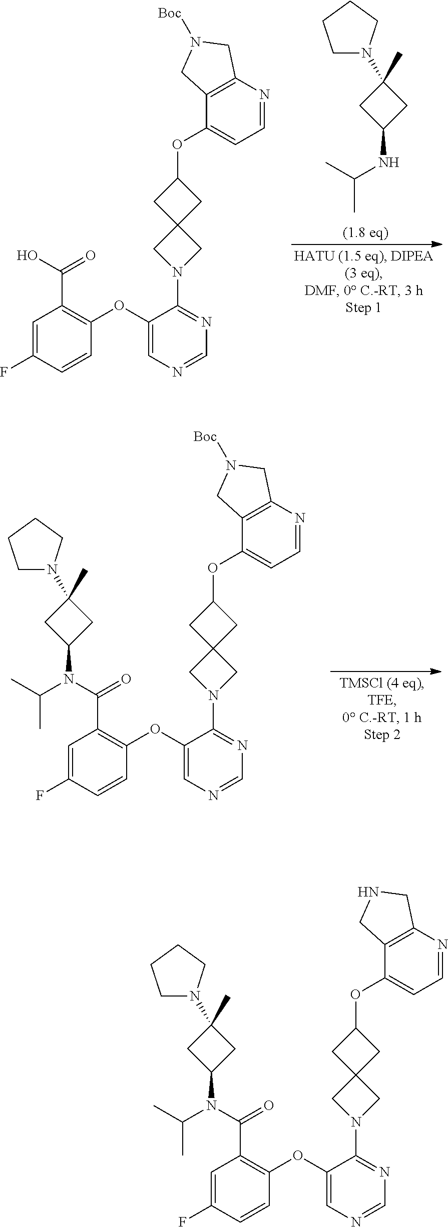 [SMILES: CC(C)(C)OC(=O)N1Cc2nccc(OC3CC4(C3)CN(c3ncncc3Oc3ccc(F)cc3C(=O)O)C4)c2C1 ; CC(C)N[C@H]1C[C@](C)(N2CCCC2)C1 ; CC(C)N(C(=O)c1cc(F)ccc1Oc1cncnc1N1CC2(CC(Oc3ccnc4c3CN(C(=O)OC(C)(C)C)C4)C2)C1)[C@H]1C[C@](C)(N2CCCC2)C1 ; CC(C)N(C(=O)c1cc(F)ccc1Oc1cncnc1N1CC2(CC(Oc3ccnc4c3CNC4)C2)C1)[C@H]1C[C@](C)(N2CCCC2)C1]

### Step 1. tert-Butyl 4-((2-(5-(4-fluoro-2-(isopropyl((1r,3r)-3-methyl-3-(pyrrolidin-1-yl)cyclobutyl)carbamoyl)phenoxyl)pyrimidin-4-yl)-2-azaspiro[3.3]heptan-6-yl)oxy)-5,7-dihydro-6H-pyrrolo[3,4-b]pyridine-6-carboxylate

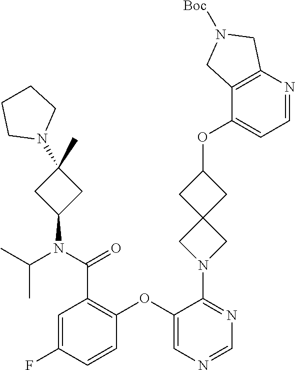 [SMILES: CC(C)N(C(=O)c1cc(F)ccc1Oc1cncnc1N1CC2(CC(Oc3ccnc4c3CN(C(=O)OC(C)(C)C)C4)C2)C1)[C@H]1C[C@](C)(N2CCCC2)C1]

This compound was synthesized following the procedure described for the synthesis of Example 1, step 2, starting with 1 equivalent of 2-((4-(6-((6-(tert-butoxycarbonyl)-6,7-dihydro-5H-pyrrolo[3,4-b]pyridin-4-yl)oxy)-2-azaspiro[3.3]heptan-2-yl)pyrimidin-5-yl)oxy)-5-fluorobenzoic acid and 1.8 equivalents of (1r,3r)—N-isopropyl-3-methyl-3-(pyrrolidin-1-yl)cyclobutan-1-amine. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl 4-((2-(5-(4-fluoro-2-(isopropyl((1r,3r)-3-methyl-3-(pyrrolidin-1-yl)cyclobutyl)carbamoyl)phenoxyl)pyrimidin-4-yl)-2-azaspiro[3.3]heptan-6-yl)oxy)-5,7-dihydro-6H-pyrrolo[3,4-b]pyridine-6-carboxylate as a solid.

Yield: 62.4%; ¹H NMR (400 MHz, DMSO-dh): S 8.31 (br d, J=5.5 Hz, 1H), 8.28 (s, 1H), 7.81-7.56 (m, 1H), 7.35-7.03 (m, 3H), 6.85-6.73 (m, 1H), 4.83 (quin, J=6.5 Hz, 1H), 4.56-4.46 (m, 4H), 4.36-4.01 (m, 5H), 3.89-3.63 (m, 2H), 2.89-2.76 (m, 3H), 2.66-2.62 (m, 1H), 2.37 (br s, 2H), 2.32-2.07 (m, 4H), 1.86-1.75 (m, 1H), 1.72-1.64 (m, 2H), 1.64-1.53 (m, 2H), 1.46 (s, 9H), 1.35-0.88 (m, 9H); LCMS (Method A): Rt=2.11 min, m/z=742.3 [M+H]⁺, 93.96%.

### Step 2. 2-((4-(6-((6,7-Dihydro-5H-pyrrolo[3,4-b pyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl) cyclobutyl)benzamide

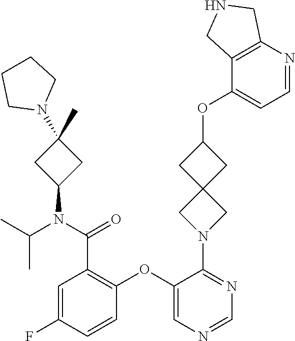 [SMILES: CC(C)N(C(=O)c1cc(F)ccc1Oc1cncnc1N1CC2(CC(Oc3ccnc4c3CNC4)C2)C1)[C@H]1C[C@](C)(N2CCCC2)C1]

This compound was synthesized following the procedure described for the synthesis of Example 20, step 3, starting with 1 equivalent of tert-butyl 4-((2-(5-(4-fluoro-2-(isopropyl((1r,3r)-3-methyl-3-(pyrrolidin-1-yl)cyclobutyl)carbamoyl)phenoxyl)pyrimidin-4-yl)-2-azaspiro[3.3]heptan-6-yl)oxy)-5,7-dihydro-6H-pyrrolo[3,4-b]pyridine-6-carboxylate. After completion, the reaction was concentrated under reduced pressure to obtain 2-((4-(6-((6,7-dihydro-5H-pyrrolo[3,4-b]pyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide xHCl. The crude was purified by reverse phase chromatography using a SiliCycle SiliaSep C18 column (50 g) on Biotage isolera one (Mobile phase A: 10 mM NH₄HCO₃ in water, B: ACN) to obtain 2-((4-(6-((6,7-dihydro-5H-pyrrolo[3,4-b]pyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide as a solid.

Yield: 74.2% yield; ¹H NMR (400 MHz, DMSO-dh): S 8.27 (s, 1H), 8.21 (br d, J=5.5 Hz, 1H), 7.80-7.57 (m, 1H), 7.32-7.04 (m, 3H), 6.80-6.63 (m, 1H), 4.90-4.73 (m, 1H), 4.59-4.43 (m, 1H), 4.36-3.96 (m, 7H), 3.89-3.64 (m, 2H), 2.89-2.74 (m, 3H), 2.41-2.35 (m, 2H), 2.31-2.07 (m, 4H), 2.02-1.90 (m, 1H), 1.86-1.75 (m, 1H), 1.73-1.64 (m, 2H), 1.62-1.54 (m, 2H), 1.48-0.87 (m, 9H), two protons merged with solvent peaks; LCMS (Method H): Rt=1.30 min, m/z=642.4 [M+H]⁺; HPLC (Method G): Rt=6.50 min, 99.28%; Chiral SFC (Method J) Rt=5.01 min, 98.51%.

### Example 139. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-azaspiro[3.4]octan-2-yl)benzamide (Compound No. 139)

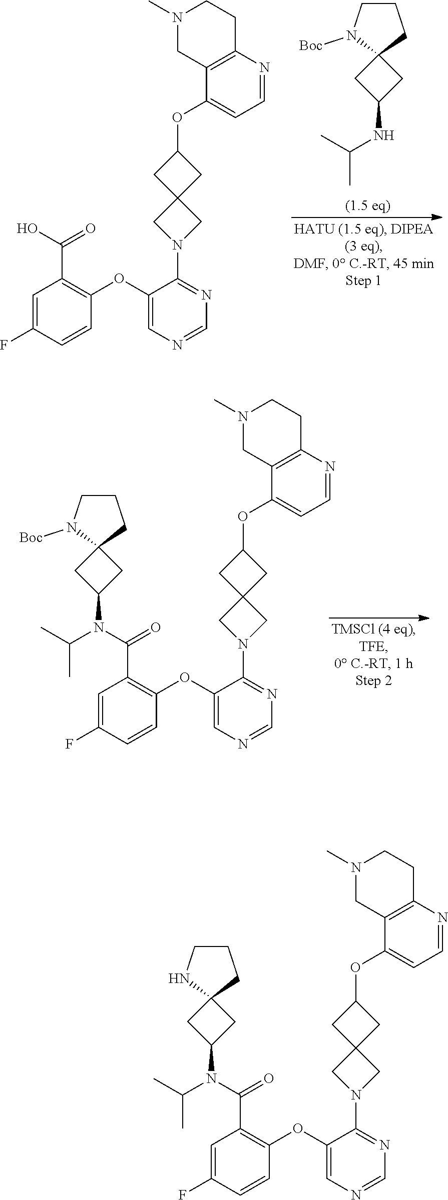 [SMILES: CN1CCc2nccc(OC3CC4(C3)CN(c3ncncc3Oc3ccc(F)cc3C(=O)O)C4)c2C1 ; CC(C)N[C@H]1C[C@@]2(CCCN2C(=O)OC(C)(C)C)C1 ; CC(C)N(C(=O)c1cc(F)ccc1Oc1cncnc1N1CC2(CC(Oc3ccnc4c3CN(C)CC4)C2)C1)[C@H]1C[C@@]2(CCCN2C(=O)OC(C)(C)C)C1 ; CC(C)N(C(=O)c1cc(F)ccc1Oc1cncnc1N1CC2(CC(Oc3ccnc4c3CN(C)CC4)C2)C1)[C@H]1C[C@@]2(CCCN2)C1]

### Step 1. tert-Butyl(2s,4r)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.4]octane-5-carboxylate

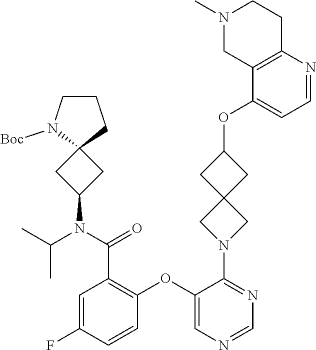 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCN2C(=O)OC(C)(C)C)C1]

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.5 equivalents of tert-butyl(2s,4r)-2-(isopropylamino)-5-azaspiro[3.4]octane-5-carboxylate. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl(2s,4r)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.4]octane-5-carboxylate as a solid.

Yield: 60.8%; ¹H NMR (400 MHz, DMSO-d₆): δ 8.27 (s, 1H), 8.18 (d, J=5.5 Hz, 1H), 7.92-7.72 (m, 1H), 7.37-6.92 (m, 3H), 6.64 (br d, J=5.5 Hz, 1H), 4.82-4.65 (m, 1H), 4.55-3.97 (m, 5H), 3.84-3.57 (m, 1H), 3.36 (br s, 2H), 3.28-3.16 (m, 3H), 3.11-2.96 (m, 1H), 2.86-2.71 (m, 5H), 2.63 (br t, J=5.3 Hz, 2H), 2.36 (br s, 3H), 2.29-2.11 (m, 4H), 1.67-1.27 (m, 12H), 1.21-0.85 (m, 6H); LCMS (Method A): Rt=2.95 min, m/z=742.3 [M+H]⁺, 96.52%.

### Step 2. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-azaspiro[3.4]octan-2-yl)benzamide

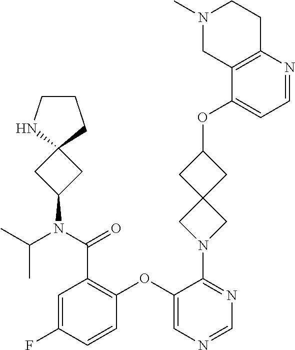 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCN2)C1]

This compound was synthesized following the procedure described for the synthesis of Example 20, step 3, starting with 1 equivalent of tert-butyl(2s,4r)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.4]octane-5-carboxylate. After completion, the reaction was concentrated under reduced pressure to obtain crude 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-azaspiro[3.4]octan-2-yl)benzamide xHCl. The crude was purified by reverse phase chromatography using a SiliCycle SiliaSep C18 column (80 g) on Biotage isolera one (Mobile phase A: 10 mM NH₄HCO₃ in water, B: ACN) to obtain 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-azaspiro[3.4]octan-2-yl)benzamide as a solid.

Yield: 21.77%; ¹H NMR (400 MHz, DMSO-d₆): δ 8.28 (s, 1H), 8.18 (d, J=5.5 Hz, 1H), 7.78-7.71 (m, 1H), 7.31-7.02 (m, 3H), 6.65 (br d, J=5.5 Hz, 1H), 4.80-4.69 (m, 1H), 4.38-4.05 (m, 5H), 3.78-3.62 (m, 1H), 3.36 (s, 2H), 3.16-2.88 (m, 1H), 2.85-2.70 (m, 5H), 2.63 (br t, J=5.8 Hz, 2H), 2.36 (s, 3H), 2.30-2.21 (m, 2H), 2.15-1.77 (m, 4H), 1.69-1.47 (m, 4H), 1.45-0.93 (m, 6H), one proton merged with solvent peaks; LCMS: Rt=1.62 min, m/z=642.3 [M+H]⁺; HPLC (Method C): Rt=7.93 min, 99.75%; Chiral SFC (Method Y): Rt=3.54 min, 100%.

### Example 140. (D1)-N-((1r,3r)-3-(Azetidin-1-yl)-3-methylcyclobutyl)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamide (Compound No. 140)

### Example 141. (D2)-N-((1r,3r)-3-(Azetidin-1-yl)-3-methylcyclobutyl)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamide (Compound No. 141)

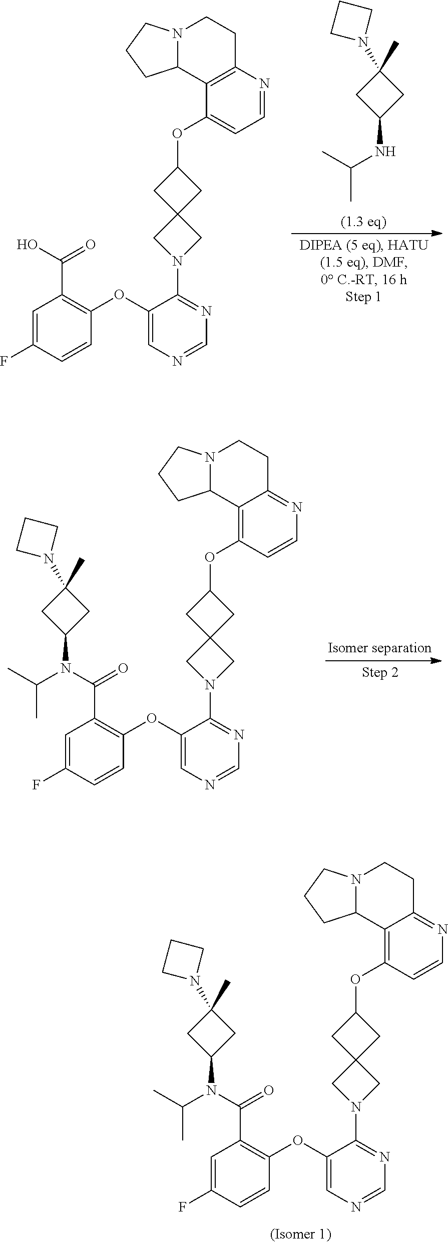 [SMILES: O=C(O)c1cc(F)ccc1Oc1cncnc1N1CC2(CC(Oc3ccnc4c3C3CCCN3CC4)C2)C1 ; CC(C)N[C@H]1C[C@](C)(N2CCC2)C1 ; CC(C)N(C(=O)c1cc(F)ccc1Oc1cncnc1N1CC2(CC(Oc3ccnc4c3C3CCCN3CC4)C2)C1)[C@H]1C[C@](C)(N2CCC2)C1 ; CC(C)N(C(=O)c1cc(F)ccc1Oc1cncnc1N1CC2(CC(Oc3ccnc4c3C3CCCN3CC4)C2)C1)[C@H]1C[C@](C)(N2CCC2)C1]

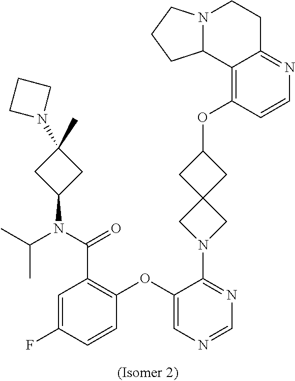 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(N2CCC2)C1]

### Step 1. N-((1r,3r)-3-(Azetidin-1-yl)-3-methylcyclobutyl)-5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropylbenzamide

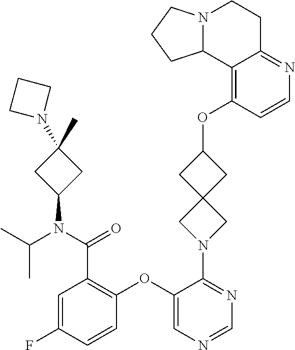 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(N2CCC2)C1]

This compound was synthesized following the procedure described for the synthesis of Example 1, step 2, starting with 1 equivalent of (±)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoic acid and 1.3 equivalents of (1r,3r)-3-(azetidin-1-yl)-N-isopropyl-3-methylcyclobutan-1-amine, except that 5 equivalents of DIPEA was used. After completion, the reaction was concentrated under reduced pressure. The crude was purified by reverse phase chromatography using a SiliCycle SiliaSep C18 column (40 g) on Biotage isolera one (Mobile phase A: 10 mM NH-4HCO₃ in water, B: ACN). The pure fractions were concentrated under reduced pressure to afford N-((1r,3r)-3-(azetidin-1-yl)-3-methylcyclobutyl)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamide as a solid.

Yield: 46.6%; LCMS (Method A): Rt=1.70 min m/z=682.3 [M+H]⁺; HPLC (Method G): Rt=6.32 min, 96.14%; Chiral SFC (Method AX): Peak 1: Rt=4.76 min, 49.98%; Peak 2: Rt=10.82 min, 48.55%.

### Step 2. (D1)-N-((1r,3r)-3-(Azetidin-1-yl)-3-methylcyclobutyl)-5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropylbenzamide and (D2)-N-((1r,3r)-3-(azetidin-1-yl)-3-methylcyclobutyl)-5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropylbenzamide

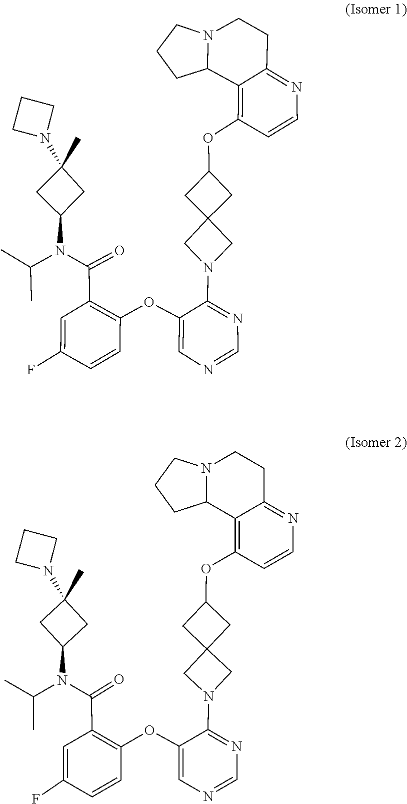 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(N2CCC2)C1 ; CC(C)N(C(=O)c1cc(F)ccc1Oc1cncnc1N1CC2(CC(Oc3ccnc4c3C3CCCN3CC4)C2)C1)[C@H]1C[C@](C)(N2CCC2)C1]

The diastereomeric mixture of N-((1r,3r)-3-(azetidin-1-yl)-3-methylcyclobutyl)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamide (300 mg, 0.440 mmol) was purified by chiral Prep SFC (Method O) to afford (D1)-N-((1r,3r)-3-(azetidin-1-yl)-3-methylcyclobutyl)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamide (49.51 mg, 16% yield) and (D2)-N-((1r,3r)-3-(azetidin-1-yl)-3-methylcyclobutyl)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamide (42.58 mg, 13.53% yield) as solids.

Isomer 1. ¹H NMR (400 MHz, DMSO-d₆): δ 8.31-8.24 (m, 1H), 8.17 (d, J=5.6 Hz, 1H), 7.79-7.60 (m, 1H), 7.33-7.05 (m, 3H), 6.70-6.61 (m, 1H), 4.75 (quin, J=6.4 Hz, 1H), 4.34-3.92 (m, 4H), 3.79-3.61 (m, 3H), 3.23-3.08 (m, 2H), 3.02-2.74 (m, 8H), 2.67-2.65 (m, 1H), 2.44-2.37 (m, 2H), 2.30-1.98 (m, 4H), 1.96-1.65 (m, 5H), 1.55-1.42 (m, 2H), 1.35-0.91 (m, 9H); LCMS (Method H): Rt=1.04 min, m/z=682.4 [M+H]⁺; HPLC (Method G): Rt=6.39 min, 96.92%; Chiral Prep SFC (Method AX): Rt=5.11 min, 100%.

Isomer 2. ¹H NMR (400 MHz, DMSO-d₆): δ 8.32-8.24 (m, 1H), 8.20-8.13 (m, 1H), 7.79-7.61 (m, 1H), 7.34-7.05 (m, 3H), 6.70-6.62 (m, 1H), 4.75 (quin, J=6.3 Hz, 1H), 4.36-3.91 (m, 4H), 3.78-3.62 (m, 3H), 3.18-3.3.06 (m, 2H), 3.01-2.73 (m, 8H), 2.67-2.61 (m, 1H), 2.43-2.36 (m, 2H), 2.31-1.99 (m, 4H), 1.94-1.66 (m, 5H), 1.53-1.41 (m, 2H), 1.36-0.90 (m, 9H); LCMS (Method H): Rt=1.04 min, m/z=682.3 [M+H]⁺; HPLC (Method F): Rt=5.99 min, 95.35%; Chiral Prep SFC (Method AX): Rt=12.41 min, 100%.

### Example 142. 5-Fluoro-2-((4-(6-(((S)-5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((1r,3r)-3-methyl-3-(pyrrolidin-1-yl)cyclobutyl)benzamide or (S)-5-Fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((1r,3r)-3-methyl-3-(pyrrolidin-1-yl)cyclobutyl)benzamide (Compound No. 142) (Method 1)

### Example 143. 5-Fluoro-2-((4-(6-(((R)-5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((1r,3r)-3-methyl-3-(pyrrolidin-1-yl)cyclobutyl)benzamide (Compound No. 143) or (R)-5-Fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((1r,3r)-3-methyl-3-(pyrrolidin-1-yl)cyclobutyl)benzamide (Method 1)

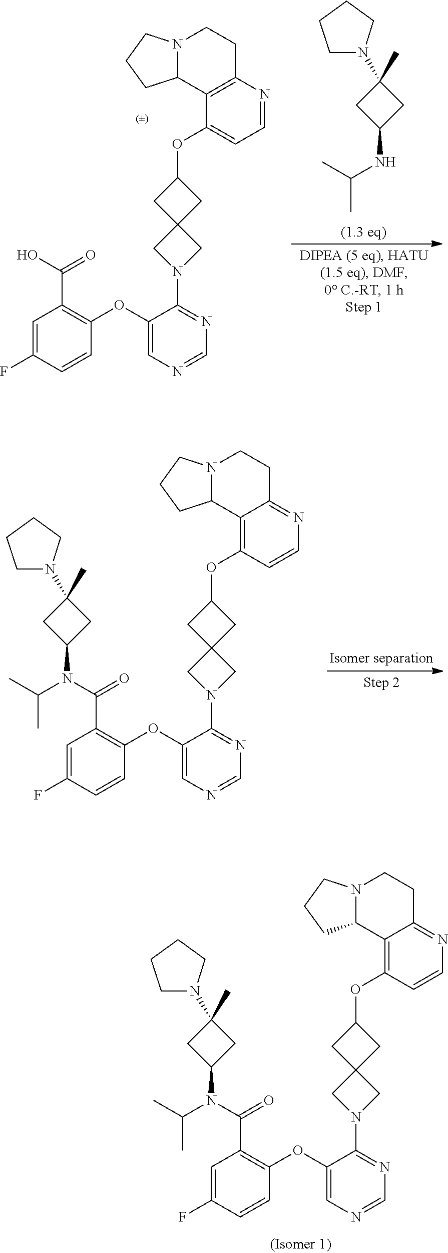 [SMILES: O=C(O)c1cc(F)ccc1Oc1cncnc1N1CC2(CC(Oc3ccnc4c3C3CCCN3CC4)C2)C1 ; CC(C)N[C@H]1C[C@](C)(N2CCCC2)C1 ; CC(C)N(C(=O)c1cc(F)ccc1Oc1cncnc1N1CC2(CC(Oc3ccnc4c3C3CCCN3CC4)C2)C1)[C@H]1C[C@](C)(N2CCCC2)C1 ; CC(C)N(C(=O)c1cc(F)ccc1Oc1cncnc1N1CC2(CC(Oc3ccnc4c3[C@@H]3CCCN3CC4)C2)C1)[C@H]1C[C@](C)(N2CCCC2)C1]

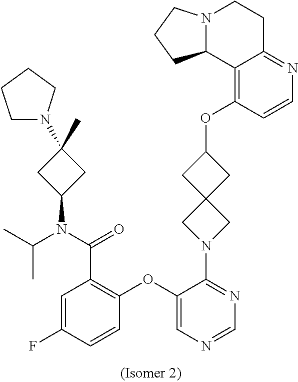 [SMILES: CC(C)N(C(=O)c1cc(F)ccc1Oc1cncnc1N1CC2(CC(Oc3ccnc4c3[C@H]3CCCN3CC4)C2)C1)[C@H]1C[C@](C)(N2CCCC2)C1]

### Step 1. 5-Fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropyl-N-((1r,3r)-3-methyl-3-(pyrrolidin-1-yl)cyclobutyl)benzamide

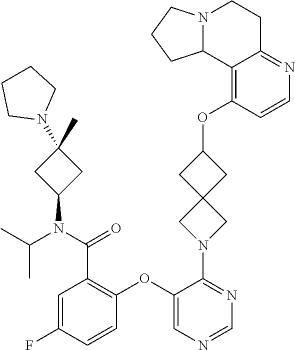 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(N2CCCC2)C1]

This compound was synthesized following the procedure described for the synthesis of Example 1, step 2, starting with 1 equivalent of (+)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoic acid and 1.3 equivalents of (1r,3r)—N-isopropyl-3-methyl-3-(pyrrolidin-1-yl)cyclobutan-1-amine, except that 5 equivalents of DIPEA was used. After completion, the reaction was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure to afford the diastereomeric mixture 5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((1r,3r)-3-methyl-3-(pyrrolidin-1-yl)cyclobutyl)benzamide as a solid.

Yield: 25.08%; LCMS (Method H): Rt=1.08 min, m/z=696.4 [M+H]⁺; HPLC (Method C): Rt=8.98 min, 97.73%; Chiral SFC (Method AX): Peak 1: Rt=4.31 min, 50.51%; Peak 2: Rt=11.01 min, 49.49%.

### Step 2. 5-Fluoro-2-((4-(6-(((S)-5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((1r,3r)-3-methyl-3-(pyrrolidin-1-yl)cyclobutyl)benzamide and 5-fluoro-2-((4-(6-(((R)-5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropyl-N-((1r,3r)-3-methyl-3-(pyrrolidin-1-yl)cyclobutyl)benzamide

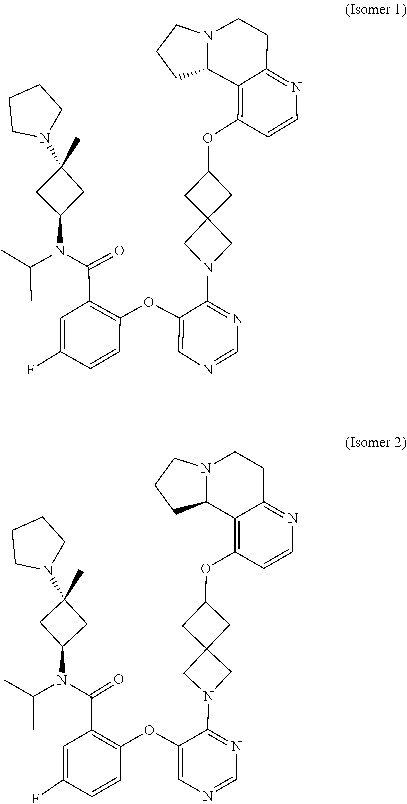 [SMILES: CC(C)N(C(=O)c1cc(F)ccc1Oc1cncnc1N1CC2(CC(Oc3ccnc4c3[C@@H]3CCCN3CC4)C2)C1)[C@H]1C[C@](C)(N2CCCC2)C1 ; CC(C)N(C(=O)c1cc(F)ccc1Oc1cncnc1N1CC2(CC(Oc3ccnc4c3[C@H]3CCCN3CC4)C2)C1)[C@H]1C[C@](C)(N2CCCC2)C1]

The diastereomeric mixture of 5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((1r,3r)-3-methyl-3-(pyrrolidin-1-yl)cyclobutyl)benzamide (170 mg, 0.244 mmol) was purified by chiral Prep SFC (Method 0), then both isomers were individually purified by reverse phase chromatography using a SiliCycle SiliaSep C18 column (12 g) on Biotage isolera one (Mobile phase A: 10 mM NH₄HCO₃ in water, B: ACN) to afford both isomers as solids.

Isomer 1: 5-Fluoro-2-((4-(6-(((S)-5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((1r,3r)-3-methyl-3-(pyrrolidin-1-yl)cyclobutyl)benzamide (30 mg, 17.46% yield). ¹H NMR (400 MHz, DMSO-d₆): δ 8.30-8.23 (m, 1H), 8.17 (d, J=5.5 Hz, 1H), 7.78-7.57 (m, 1H), 7.31-7.06 (m, 3H), 6.70-6.61 (m, 1H), 4.75 (quin, J=6.5 Hz, 1H), 4.34-4.00 (m, 5H), 3.88-3.60 (m, 3H), 3.01-2.73 (m, 7H), 2.71-2.61 (m, 3H), 2.42-2.35 (m, 2H), 2.29-1.92 (m, 4H), 1.85-1.56 (m, 7H), 1.52-0.91 (m, 11H); LCMS (Method H): Rt=1.06 min, m/z=696.4 [M+H]⁺; HPLC (Method C): Rt=8.64 min, 98.94%; Chiral Prep SFC (Method AX): Rt=4.42 min, 100%.

Isomer 2: 5-Fluoro-2-((4-(6-(((R)-5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((1r,3r)-3-methyl-3-(pyrrolidin-1-yl)cyclobutyl)benzamide (25 mg, 14.68% yield). ¹H NMR (400 MHz, DMSO-d₆): δ 8.31-8.22 (m, 1H), 8.16 (br d, J=5.4 Hz, 1H), 7.78-7.58 (m, 1H), 7.31-7.06 (m, 3H), 6.69-6.62 (m, 1H), 4.75 (quin, J=6.3 Hz, 1H), 4.34-4.03 (m, 5H), 3.91-3.61 (m, 3H), 3.01-2.73 (m, 7H), 2.66-2.60 (m, 3H), 2.43-2.36 (m, 2H), 2.29-1.92 (m, 4H), 1.86-1.56 (m, 7H), 1.52-0.90 (m, 11H); LCMS (Method H): Rt=1.07 min, m/z=696.3 [M+H]⁺; HPLC (Method C): Rt=8.61 min, 99.84%; Chiral Prep SFC (Method AX): Peak 1: Rt=13.19 min, 100%.

### Example 142. (S)-5-Fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((1r,3r)-3-methyl-3-(pyrrolidin-1-yl)cyclobutyl)benzamide (Compound No. 142) (Method 2)

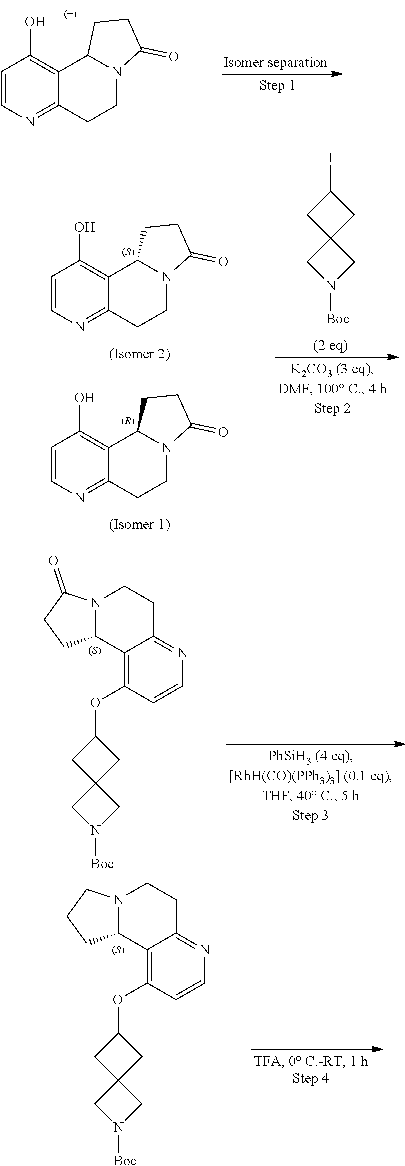

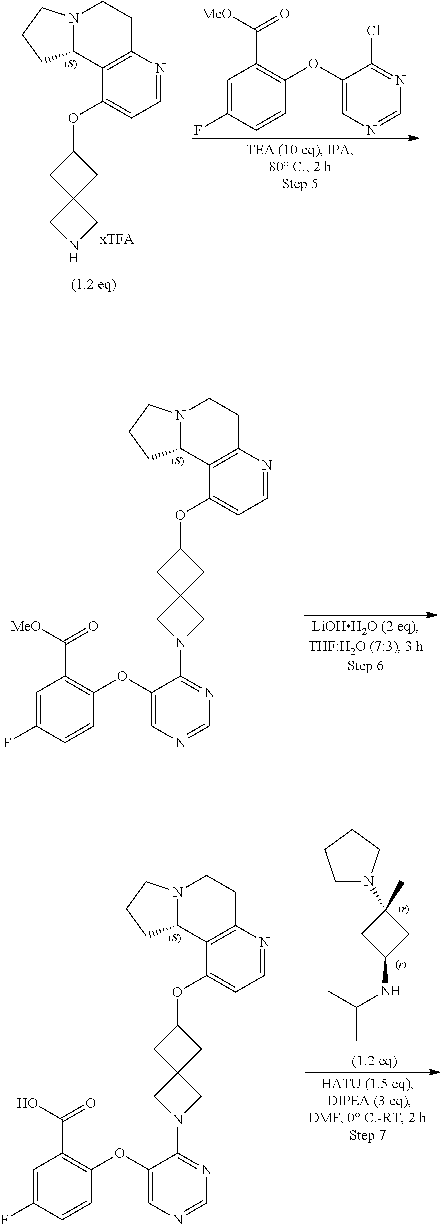

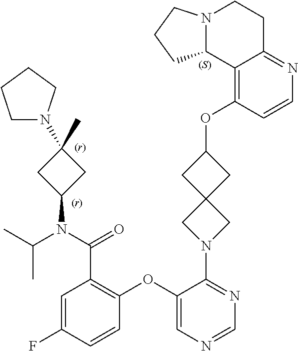

### Step 1. (R)-1-Hydroxy-5,9,10,10α-tetrahydropyrrolo[2,1-f][1,6]naphthyridin-8(6H)-one and(S)-1-hydroxy-5,9,10,10α-tetrahydropyrrolo[2,1-f][1,6]naphthyridin-(6H)-one

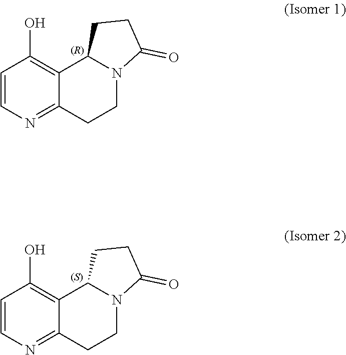

(±)-1-Hydroxy-5,9,10,10α-tetrahydropyrrolo[2,1-f][1,6]naphthyridin-8(6H)-one (122 g, 597 mmol) was purified by chiral Prep SFC (Method Y). Both fractions were concentrated under reduced pressure to afford both isomers as solids.

Isomer 1. (R)-1-Hydroxy-5,9,10,10a-tetrahydropyrrolo[2,1-f][1,6]naphthyridin-8(6H)-one (59.17 g, 46.26% Yield). ¹H NMR (400 MHz, DMSO-d₆): δ 11.19-9.11 (m, 1H), 7.57 (d, J=7.1 Hz, 1H), 6.03 (d, J=7.1 Hz, 1H), 4.51 (br t, J=7.8 Hz, 1H), 4.13 (dd, J=5.3, 12.9 Hz, 1H), 2.93-2.75 (m, 2H), 2.68-2.55 (m, 2H), 2.46-2.31 (m, 1H), 2.15 (dd, J=8.4, 16.5 Hz, 1H), 1.50-1.36 (m, 1H); LCMS (Method B): Rt=0.32 min, m/z=205.6 [M+H]⁺; HPLC (Method G): Rt=3.31 min, 95.39%; Chiral SFC (Method AAK): Rt=6.94 min, 96.97%; [α]_{D}²⁵=+510.84° (c 0.50, MeOH).

Isomer 2. (S)-1-Hydroxy-5,9,10,10a-tetrahydropyrrolo[2,1-f][1,6]naphthyridin-8(6H)-one (45.9 g, 42.69% Yield). ¹H NMR (400 MHz, DMSO-d₆): δ 11.53-10.84 (m, 1H), 7.56 (br d, J=6.3 Hz, 1H), 6.02 (br s, 1H), 4.50 (br t, J=7.6 Hz, 1H), 4.14 (br dd, J=5.8, 12.8 Hz, 1H), 2.93-2.73 (m, 2H), 2.68-2.54 (m, 2H), 2.46-2.33 (m, 1H), 2.15 (br dd, J=8.8, 16.4 Hz, 1H), 1.49-1.36 (m, 1H); LCMS (Method B): Rt=0.31 min, m/z=205.2 [M+H]⁺; HPLC (Method G): Rt=3.29 min, 98.61%; Chiral SFC (Method AAK): Rt=7.94 min, 100%; [α]_{D}²⁵=−549.84° (c 0.50, MeOH).

### Step 2. (S)-tert-Butyl 6-((8-oxo-5,6,8,9,10,10α-hexahydropyrrolo[2,1-f][1,6]naphthyridin-1-yl)oxy)-2-azaspiro[3.3]heptane-2-carboxylate

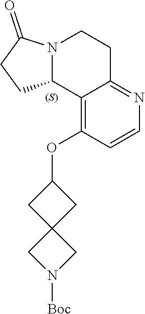

This compound was synthesized following the procedure described for the synthesis of Intermediate 18, step 3, starting with 1 equivalent of (S)-1-hydroxy-5,9,10,10a-tetrahydropyrrolo[2,1-f][1,6]naphthyridin-8(6H)-one and 2 equivalents of tert-butyl 6-iodo-2-azaspiro[3.3]heptane-2-carboxylate. After completion, the reaction was quenched with water, and the mixture was extracted with EtOAc. The combined organic layer was washed with ice-cold brine solution, dried over Na₂SO₄ and filtered, and the filtrate was concentrated under reduced pressure. The crude was purified by silica gel flash column chromatography using MeOH in DCM (product eluted at 10% MeOH in DCM) to obtain (S)-tert-butyl 6-((8-oxo-5,6,8,9,10,10a-hexahydropyrrolo[2,1-f][1,6]naphthyridin-1-yl)oxy)-2-azaspiro[3.3]heptane-2-carboxylate as a solid.

Yield: 63.95%; ¹H NMR (400 MHz, DMSO-d₆): δ 8.25 (d, J=5.6 Hz, 1H), 6.77 (d, J=5.8 Hz, 1H), 4.81-4.71 (m, 2H), 4.23-4.15 (m, 1H), 3.91 (br s, 2H), 3.85 (br s, 2H), 2.99-2.90 (m, 1H), 2.80-2.70 (m, 5H), 2.48-2.41 (m, 1H), 2.31-2.18 (m, 3H), 1.65-1.52 (m, 1H), 1.37 (s, 9H); LCMS (Method A): Rt=1.64 min, m/z=400.2 [M+H]⁺; HPLC (Method C): Rt=8.67 min, 93.76%; Chiral SFC (Method 1): Rt=2.75 min, 90.34%.

### Step 3. (S)-tert-Butyl 6-((5,6,8,9,10,10α-hexahydropyrrolo[2,1-f][1,6]naphthyridin-1-yl)oxy)-2-azaspiro[3.3]heptane-2-carboxylate

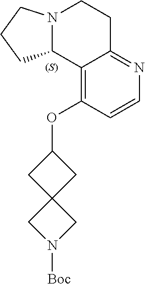

This compound was synthesized following the procedure described for the synthesis of Intermediate 19 (Procedure 2), step 1, starting with 1 equivalent of (S)-tert-butyl 6-((8-oxo-5,6,8,9,10,10a-hexahydropyrrolo[2,1-f][1,6]naphthyridin-1-yl)oxy)-2-azaspiro[3.3]heptane-2-carboxylate. After work-up, the crude compound was purified by reverse phase chromatography using a SiliCycle SiliaSep C18 column (120 g) on Biotage isolera one (Mobile phase A: 0.1% formic acid in water, B: ACN). The pure fractions were concentrated under reduced pressure. The residue was treated with saturated sodium bicarbonate solution and the mixture was extracted with DCM. The combined organic layer was dried over sodium sulfate and filtered, and the filtrate was concentrated under reduced pressure to obtain (S)-tert-butyl 6-((5,6,8,9,10,10a-hexahydropyrrolo[2,1-f][1,6]naphthyridin-1-yl)oxy)-2-azaspiro[3.3]heptane-2-carboxylate as a solid.

Yield: 51.8%; ¹H NMR (400 MHz, DMSO-d₆): δ 8.18 (d, J=5.5 Hz, 1H), 6.66 (d, J=5.6 Hz, 1H), 4.71 (quin, J=6.5 Hz, 1H), 3.91 (br s, 2H), 3.84 (br s, 2H), 3.64 (t, J=8.3 Hz, 1H), 3.01-2.83 (m, 3H), 2.79-2.66 (m, 5H), 2.43-2.37 (m, 1H), 2.27-2.13 (m, 2H), 1.88-1.70 (m, 2H), 1.55-1.43 (m, 1H), 1.37 (s, 9H); LCMS (Method H): Rt=1.28 min, m/z=386.2 [M+H]⁺; HPLC (Method H): Rt=6.26 min, 99.05%; Chiral SFC (Method N): Rt=1.16 min, 100/.

### Step 4. (S)-1-((2-Azaspiro[3.3]heptan-6-yl)oxy)-5,6,8,9,10,10α-hexahydropyrrolo[2,1-f][1,6]naphthyridine xTFA

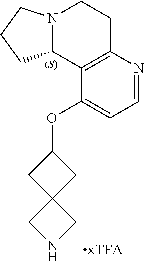

(S)-tert-Butyl 6-((5,6,8,9,10,10a-hexahydropyrrolo[2,1-f][1,6]naphthyridin-1-yl)oxy)-2-azaspiro[3.3]heptane-2-carboxylate (2.15 g, 5.58 mmol) was dissolved in TFA (6 mL) at 0° C. The reaction was stirred at RT for 1 h. The reaction was monitored by LCMS and TLC (10% MeOH in DCM). After completion, the reaction was concentrated under reduced pressure to obtain crude (S)-1-((2-azaspiro[3.3]heptan-6-yl)oxy)-5,6,8,9,10,10a-hexahydropyrrolo[2,1-f][1,6]naphthyridine xTFA (5.86 g, quantitative yield) as a sticky liquid. This material was used without further purification. ¹H NMR (400 MHz, DMSO-d₆): δ 11.46-10.84 (m, 1H), 8.88-8.71 (m, 2H), 8.67 (d, J=6.4 Hz, 1H), 7.23 (d, J=6.4 Hz, 1H), 4.98 (quin, J=6.7 Hz, 1H), 4.78 (br t, J=8.9 Hz, 1H), 4.11-3.96 (m, 4H), 3.71-3.42 (m, 4H), 3.30-3.18 (m, 2H), 2.94-2.80 (m, 2H), 2.75-2.64 (m, 1H), 2.46-2.31 (m, 2H), 2.15-2.04 (m, 2H), 1.94-1.83 (m, 1H); Chiral SFC (Method I): Rt=2.62 min, 91.89%.

### Step 5. (S)-Methyl 5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)benzoate

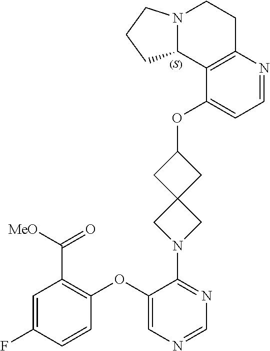

To a stirred solution of (S)-1-((2-azaspiro[3.3]heptan-6-yl)oxy)-5,6,8,9,10,10a-hexahydropyrrolo[2,1-f][1,6]naphthyridine xTFA (5.86 g, 9.34 mmol)) in IPA (60 mL), TEA (10.85 mL, 78 mmol) and methyl 2-((4-chloropyrimidin-5-yl)oxy)-5-fluorobenzoate (2.2 g, 7.78 mmol) were added at RT under a nitrogen atmosphere. The reaction was stirred at 80° C. for 2 h, and the progress was monitored by LCMS and TLC (10% MeOH in DCM). After completion, the reaction was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN). The pure fractions were concentrated under reduced pressure to afford (S)-methyl 5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoate (1.8 g, 42.8% yield) as a solid. ¹H NMR (400 MHz, DMSO-dh): S 8.30 (s, 1H), 8.17 (d, J=5.6 Hz, 1H), 7.70-7.64 (m, 2H), 7.53-7.45 (m, 1H), 7.11 (dd, J=4.4, 9.1 Hz, 1H), 6.67 (d, J=5.6 Hz, 1H), 4.75 (quin, J=6.4 Hz, 1H), 4.26 (s, 2H), 4.19 (s, 2H), 3.81 (s, 3H), 3.63 (brt, J=8.1 Hz, 1H), 2.99-2.83 (m, 3H), 2.81-2.73 (m, 3H), 2.68-2.60 (m, 2H), 2.42-2.35 (m, 1H), 2.31-2.20 (m, 2H), 1.84-1.64 (m, 2H), 1.53-1.39 (m, 1H); LCMS (Method A): Rt=1.74 min, m/z=532.1 [M+H]⁺; HPLC (Method C): Rt=8.76 min, 96.37%; Chiral SFC (Method AAM): Rt=5.69 min, 92.72%.

### Step 6. (S)-5-Fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)benzoic Acid

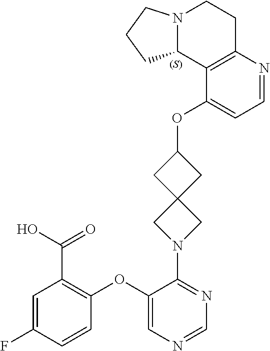

To a stirred solution of (S)-methyl 5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoate (1.8 g, 3.39 mmol) in THF (13 mL) and water (5.57 mL), LiOH·H₂O (0.284 g, 6.77 mmol) was added at 0° C. The reaction was stirred at RT for 3 h, and the progress was monitored by TLC (10% MeOH in DCM). After completion, the reaction was concentrated under reduced pressure. The crude product was cooled to 0° C. and acidified with 1 N HCl. To this mixture, water (10 mL) was added, and the resulting mixture was neutralized (pH˜7) with solid NaHCO₃. The solid was filtered and dried under vacuum to obtain some of the (S)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoic acid (300 mg, crude) as a solid. The remainder of the product was found in the aqueous layer. Therefore, the aqueous layer was concentrated under reduced pressure, and the residue was dissolved in 10% MeOH in DCM. The mixture was concentrated under reduced pressure. The residue from extraction was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure to afford the product (1.195 g). 1.195 g and 300 mg were combined to obtain (S)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoic acid (1.495 g, 81% yield) as a solid. LCMS (Method A): Rt=1.20 min, m/z=518.1 [M+H]⁺, 94.41%.

### Step 7. (S)-5-Fluoro-2-((4-(6-((5,6,8,9,10,10α-hexahydropyrrolo[2,1-f][1,6]naphthyridin-1yl)oxy)-2-azaspiro[3.3]heptan-2-yl)pyrimidin-5-yl)oxy)-N-isopropyl-N-((1r,3r)-3-methyl-3-(pyrrolidin-1-yl)cyclobutyl)benzamide

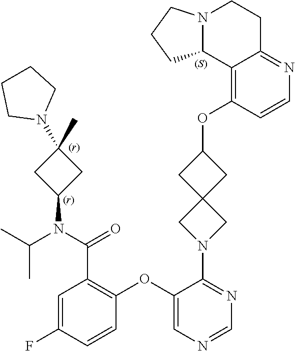

This compound was synthesized following the procedure described for the synthesis of Example 1, step 2, starting with 1 equivalent of (S)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoic acid and 1.2 equivalents of (1r,3r)—N-isopropyl-3-methyl-3-(pyrrolidin-1-yl)cyclobutan-1-amine. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to afford (D2)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((1r,3r)-3-methyl-3-(pyrrolidin-1-yl)cyclobutyl)benzamide. This compound had 13% of isomer 2. Therefore, the compound was re-purified by chiral Prep SFC (Method Z). The pure fractions were concentrated under reduced pressure and lyophilized to afford (S)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((1r,3r)-3-methyl-3-(pyrrolidin-1-yl)cyclobutyl)benzamide as a solid.

Yield: 5.21%; ¹H NMR (400 MHz, DMSO-d₆): δ 8.31-8.23 (m, 1H), 8.17 (d, J=5.5 Hz, 1H), 7.79-7.57 (m, 1H), 7.32-7.06 (m, 3H), 6.71-6.59 (m, 1H), 4.75 (quin, J=6.2 Hz, 1H), 4.35-4.04 (m, 5H), 3.90-3.62 (m, 3H), 3.01-2.73 (m, 7H), 2.71-2.61 (m, 3H), 2.42-2.36 (m, 2H), 2.30-2.17 (m, 3H), 2.14-1.91 (m, 1H), 1.85-1.56 (m, 7H), 1.52-1.42 (m, 2H), 1.34-0.90 (m, 9H); LCMS (Method H): Rt=1.09 min, m/z=696.4 [M+H]⁺; HPLC (Method G): Rt=6.45 min, 99.47%; Chiral SFC (Method AZ): Rt=7.47 min, 100%.

### Example 143. (R)-5-Fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((1r,3r)-3-methyl-3-(pyrrolidin-1-yl)cyclobutyl)benzamide (Compound No. 143) (Method 2)

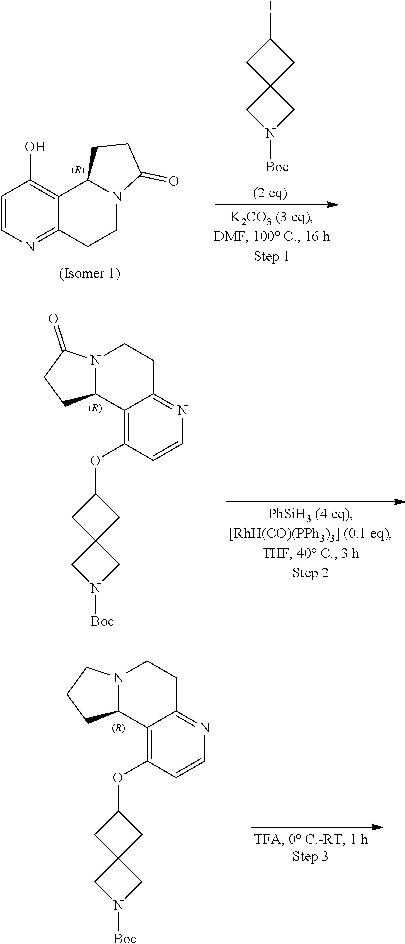

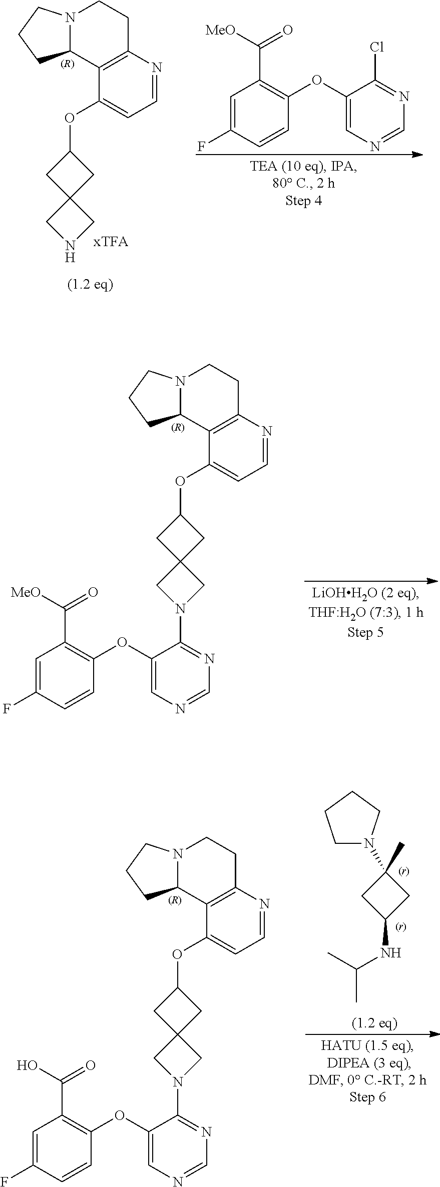

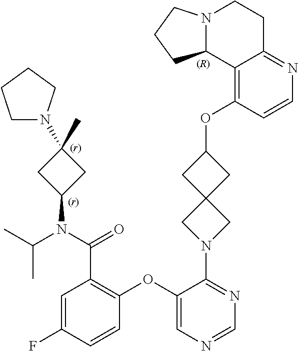

### Step 1. (R)-tert-Butyl 6-((8-oxo-5,6,8,9,10,10α-hexahydropyrrolo[2,1-f][1,6]naphthyridin-1-yl)oxy)-2-azaspiro[3.3]heptane-2-carboxylate

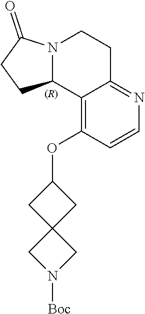

This compound was synthesized following the procedure described for the synthesis of Intermediate 18, step 3, starting with 1 equivalent of (R)-1-hydroxy-5,9,10,10a-tetrahydropyrrolo[2,1-f][1,6]naphthyridin-8(6H)-one and 2 equivalents of tert-butyl 6-iodo-2-azaspiro[3.3]heptane-2-carboxylate. After work-up, the crude was purified by silica gel flash column chromatography using MeOH in DCM (product eluted at 12% MeOH in DCM) to obtain (R)-tert-butyl 6-((8-oxo-5,6,8,9,10,10a-hexahydropyrrolo[2,1-f][1,6]naphthyridin-1-yl)oxy)-2-azaspiro[3.3]heptane-2-carboxylate as a solid.

Yield: 55.2%; ¹H NMR (400 MHz, DMSO-d₆): δ 8.25 (d, J=5.6 Hz, 1H), 6.77 (d, J=5.6 Hz, 1H), 4.81-4.71 (m, 2H), 4.24-4.15 (m, 1H), 3.91 (br s, 2H), 3.85 (br s, 2H), 2.98-2.91 (m, 1H), 2.81-2.70 (m, 5H), 2.48-2.41 (m, 1H), 2.30-2.18 (m, 3H), 1.66-1.51 (m, 1H), 1.37 (s, 9H); LCMS (Method B): Rt=1.34 min, m/z=400.4 [M+H]⁺, 93.44%; Chiral SFC (Method AAL): Rt=3.94 min, 95.09%.

### Step 2. (R)-tert-Butyl 6-((5,6,8,9,10,10α-hexahydropyrrolo[2,1-f][1,6]naphthyridin-1-yl)oxy)-2-azaspiro[3.3]heptane-2-carboxylate

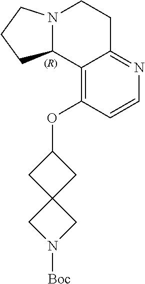

This compound was synthesized following the procedure described for the synthesis of Intermediate 19, step 1, starting with 1 equivalent of (R)-tert-butyl 6-((8-oxo-5,6,8,9,10,10a-hexahydropyrrolo[2,1-f][1,6]naphthyridin-1-yl)oxy)-2-azaspiro[3.3]heptane-2-carboxylate. After work-up, the crude compound was purified by reverse phase chromatography using a SiliCycle SiliaSep C18 column (120 g) on Biotage isolera one (Mobile phase A: 0.1% formic acid in water, B: ACN). The pure fractions were concentrated under reduced pressure. The residue was treated with saturated sodium bicarbonate solution and the mixture was extracted with 10% MeOH in DCM. The combined organic layer was dried over sodium sulfate and filtered, and the filtrate was concentrated under reduced pressure to obtain (R)-tert-butyl 6-((5,6,8,9,10,10a-hexahydropyrrolo[2,1-f][1,6]naphthyridin-1-yl)oxy)-2-azaspiro[3.3]heptane-2-carboxylate as a sticky solid.

Yield: 52.8%; ¹H NMR (400 MHz, DMSO-d₆): δ 8.18 (d, J=5.7 Hz, 1H), 6.66 (d, J=5.7 Hz, 1H), 4.71 (quin, J=6.5 Hz, 1H), 3.91 (br s, 2H), 3.84 (br s, 2H), 3.64 (br t, J=8.4 Hz, 1H), 3.02-2.82 (m, 3H), 2.79-2.61 (m, 5H), 2.43-2.36 (m, 1H), 2.25-2.14 (m, 2H), 1.86-1.68 (m, 2H), 1.54-1.42 (m, 1H), 1.39-1.33 (m, 9H); LCMS (Method B): Rt=1.09 min, m/z=386.2 [M+H]⁺; HPLC (Method G): Rt=7.30 min, 98.73%; Chiral SFC (Method AZ): Rt=2.59 min, 99.24%.

### Step 3. (R)-1-((2-Azaspiro[3.3]heptan-6-yl)oxy)-5,6,8,9,10,10α-hexahydropyrrolo[2,1-f][1,6]naphthyridine xTFA

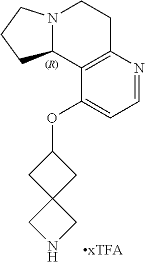

(R)-tert-Butyl 6-((5,6,8,9,10,10a-hexahydropyrrolo[2,1-f][1,6]naphthyridin-1-yl)oxy)-2-azaspiro[3.3]heptane-2-carboxylate (1.6 g, 4.15 mmol) was dissolved in TFA (4 mL) at 0° C. The reaction was stirred at RT for 1 h. The reaction was monitored by LCMS and TLC (10% MeOH in DCM). After completion, the reaction was concentrated under reduced pressure. The crude was reconcentrated from EtOAc (30 mL) and the residue was dried under vacuum to obtain crude (R)-1-((2-azaspiro[3.3]heptan-6-yl)oxy)-5,6,8,9,10,10a-hexahydropyrrolo[2,1-f][1,6]naphthyridine xTFA (4.01 g, quantitative yield) as a gummy compound. This material was used without further purification. ¹H NMR (400 MHz, DMSO-d₆): δ 11.09 (br s, 1H), 8.77 (br s, 2H), 8.65 (d, J=6.4 Hz, 1H), 7.21 (d, J=6.5 Hz, 1H), 4.97 (quin, J=6.7 Hz, 1H), 4.78 (br t, J=8.9 Hz, 1H), 4.08-3.97 (m, 4H), 3.71-3.43 (m, 4H), 3.27-3.18 (m, 2H), 2.94-2.82 (m, 2H), 2.76-2.65 (m, 1H), 2.45-2.30 (m, 2H), 2.15-2.04 (m, 2H), 1.96-1.83 (m, 1H); LCMS (Method B): Rt=0.24 min, m/z=286.2 [M+H]⁺, 89.35%; Chiral SFC (Method I): Rt=2.72 min, 100%.

### Step 4. (R)-Methyl 5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)benzoate

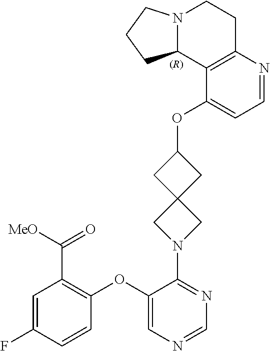

To a stirred solution of (R)-1-((2-azaspiro[3.3]heptan-6-yl)oxy)-5,6,8,9,10,10a-hexahydropyrrolo[2,1-f][1,6]naphthyridine xTFA (4.00 g, 6.37 mmol) in 2-propanol (40 mL), TEA (7.40 mL, 53.1 mmol) and methyl 2-((4-chloropyrimidin-5-yl)oxy)-5-fluorobenzoate (1.5 g, 5.31 mmol) were added at RT under a nitrogen atmosphere. The reaction was stirred at 80° C. for 2 h. The reaction was monitored by LCMS and TLC (10% MeOH in DCM). After completion, the reaction was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure to afford (R)-methyl 5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoate (1.5 g, 48.9% yield) as a solid. ¹H NMR (400 MHz, DMSO-d₆): δ 8.30 (s, 1H), 8.17 (d, J=5.5 Hz, 1H), 7.70-7.64 (m, 2H), 7.52-7.46 (m, 1H), 7.11 (dd, J=4.4, 9.1 Hz, 1H), 6.67 (d, J=5.6 Hz, 1H), 4.75 (quin, J=6.4 Hz, 1H), 4.26 (s, 2H), 4.19 (s, 2H), 3.81 (s, 3H), 3.66 (br t, J=8.2 Hz, 1H), 3.02-2.83 (m, 3H), 2.82-2.73 (m, 3H), 2.72-2.63 (m, 2H), 2.45-2.36 (m, 1H), 2.31-2.19 (m, 2H), 1.86-1.65 (m, 2H), 1.46 (quin, J=10.0 Hz, 1H); LCMS (Method A): Rt=1.74 min, m/z=532.5 [M+H]⁺, 91.96%; Chiral SFC (Method AZ): Rt=7.32 min, 93.83%.

### Step 5. (R)—5-Fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)benzoic Acid

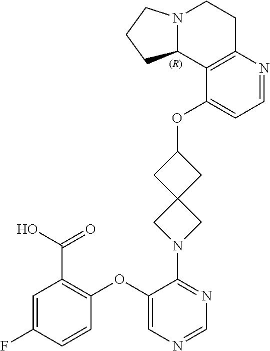

To a stirred solution of (R)-methyl 5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoate (1.5 g, 2.82 mmol) in THF (12 mL) and water (5.14 mL), LiOH·H₂O (0.237 g, 5.64 mmol) was added at 0° C. The reaction was stirred under a nitrogen atmosphere for 1 h. The reaction progress was monitored by TLC (10% MeOH in DCM). After completion, the reaction was concentrated under reduced pressure. The crude was dissolved in water (10 mL), and the solution was acidified with 1N HCl (pH˜3-4) at 0° C. The mixture was then slowly neutralized (pH˜7) using solid NaHCO₃. The resulting mixture was directly concentrated under reduced pressure, and the residue was dried under vacuum. The crude was purified by reverse phase chromatography using a SiliCycle SiliaSep C18 column (120 g) on Biotage isolera one (Mobile phase A: 10 mM NH₄HCO₃ in water, B: ACN). The pure fractions were concentrated under reduced pressure and lyophilized to afford (R)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoic acid (1.29 g, 86% yield) as a solid. LCMS (Method B): Rt=0.85 min, m/z=518.2 [M+H]⁺, 93.41%.

### Step 6. (R)-5-Fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((1r,3r)-3-methyl-3-(pyrrolidin-1-yl)cyclobutyl)benzamide

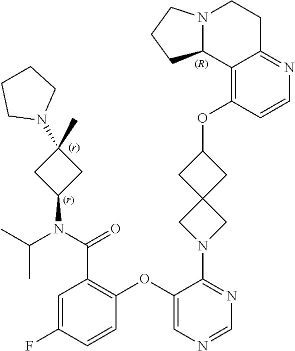

This compound was synthesized following the procedure described for the synthesis of Example 1, step 2, starting with 1 equivalent of (R)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoic acid and 1.2 equivalents of (1r,3r)—N-isopropyl-3-methyl-3-(pyrrolidin-1-yl)cyclobutan-1-amine. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford (R)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((1r,3r)-3-methyl-3-(pyrrolidin-1-yl)cyclobutyl)benzamide as a solid. This compound contained 2.3% of isomer 1.

Yield: 21.73%; ¹H NMR (400 MHz, DMSO-d₆): δ 8.31-8.23 (m, 1H), 8.17 (br d, J=5.5 Hz, 1H), 7.79-7.58 (m, 1H), 7.32-7.05 (m, 3H), 6.72-6.59 (m, 1H), 4.75 (quin, J=6.4 Hz, 1H), 4.37-4.01 (m, 5H), 3.90-3.60 (m, 3H), 3.01-2.74 (m, 7H), 2.71-2.61 (m, 3H), 2.42-2.35 (m, 2H), 2.30-2.18 (m, 3H), 2.13-1.93 (m, 1H), 1.86-1.56 (m, 7H), 1.53-1.41 (m, 2H), 1.35-0.91 (m, 9H); LCMS (Method H): Rt=1.09 min, m/z=696.4 [M+H]⁺; HPLC (Method G): Rt=6.44 min, 97.39%; Chiral SFC (Method AZ): Rt=11.40 min, 95.88%.

### Example 144. (D1)-5-Fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((2s,4r)-5-azaspiro[3.4]octan-2-yl)benzamide (Compound No. 144)

### Example 145. (D2)-5-Fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((2s,4r)-5-azaspiro[3.4]octan-2-yl)benzamide (Compound No. 145)

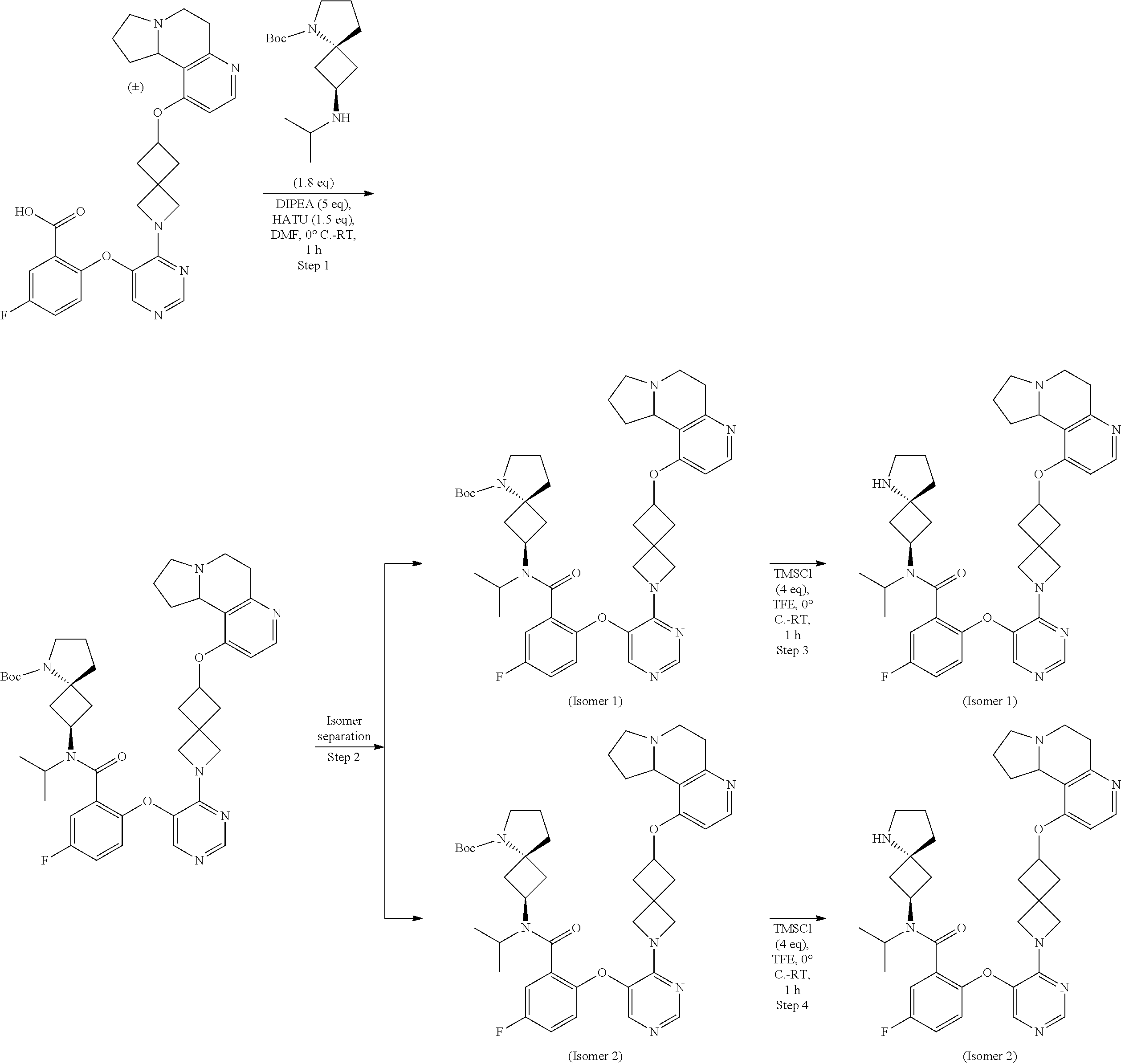

### Step 1. tert-Butyl(2s,4r)-2-(5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropylbenzamido)-5-azaspiro[3.4]octane-5-carboxylate

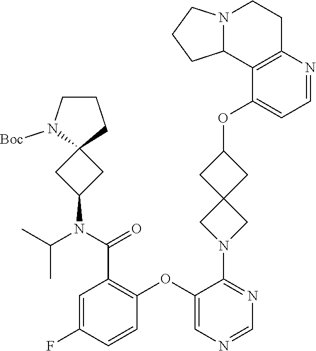 [SMILES: CC(C)N(C(=O)c1cc(F)ccc1Oc1cncnc1N1CC2(CC(Oc3ccnc4c3C3CCCN3CC4)C2)C1)[C@H]1C[C@@]2(CCCN2C(=O)OC(C)(C)C)C1]

This compound was synthesized following the procedure described for the synthesis of Example 1, step 2, starting with 1 equivalent of (±)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoic acid and 1.8 equivalents of tert-butyl(2s,4r)-2-(isopropylamino)-5-azaspiro[3.4]octane-5-carboxylate, except that 5 equivalents of DIPEA was used. After completion, the reaction was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN). The pure fractions were concentrated under reduced pressure to afford tert-butyl(2s,4r)-2-(5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamido)-5-azaspiro[3.4]octane-5-carboxylate as a solid.

Yield: 23.95%; LCMS (Method H): Rt=1.43 min, m/z=768.4 [M+H]⁺; HPLC (Method C): Rt=11.49 min, 96.05%.

### Step 2. (D1)-tert-Butyl(2s,4r)-2-(5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamido)-5-azaspiro[3.4]octane-5-carboxylate and (D2)-tert-butyl(2s,4r)-2-(5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropylbenzamido)-5-azaspiro[3.4]octane-5-carboxylate

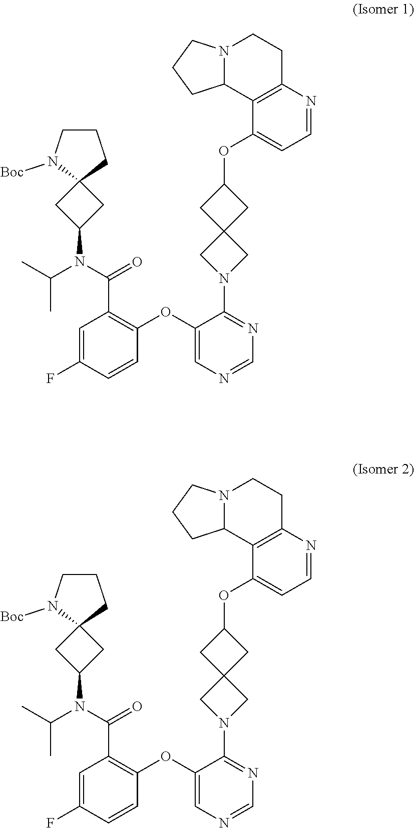 [SMILES: CC(C)N(C(=O)c1cc(F)ccc1Oc1cncnc1N1CC2(CC(Oc3ccnc4c3C3CCCN3CC4)C2)C1)[C@H]1C[C@@]2(CCCN2C(=O)OC(C)(C)C)C1 ; CC(C)N(C(=O)c1cc(F)ccc1Oc1cncnc1N1CC2(CC(Oc3ccnc4c3C3CCCN3CC4)C2)C1)[C@H]1C[C@@]2(CCCN2C(=O)OC(C)(C)C)C1]

The diastereomeric mixture of tert-butyl(2s,4r)-2-(5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamido)-5-azaspiro[3.4]octane-5-carboxylate (370 mg, 0.482 mmol) was purified by chiral Prep SFC (Method A) to obtain (D)-tert-butyl(2s,4r)-2-(5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamido)-5-azaspiro[3.4]octane-5-carboxylate (125 mg, 33.5% yield) and (D2)-tert-butyl(2s,4r)-2-(5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamido)-5-azaspiro[3.4]octane-5-carboxylate (133 mg, 33.2% yield) as solids.

Isomer 1. ¹H NMR (400 MHz, DMSO-d₆): δ 8.27 (s, 1H), 8.17 (d, J=5.7 Hz, 1H), 7.78 (s, 1H), 7.35-7.00 (m, 3H), 6.65 (d, J=5.7 Hz, 1H), 4.81-4.66 (m, 1H), 4.42-4.02 (m, 5H), 3.79-3.59 (m, 2H), 3.28-3.19 (m, 2H), 3.06-2.59 (m, 11H), 2.44-2.15 (m, 5H), 1.85-1.66 (m, 2H), 1.61-1.29 (m, 13H), 1.20-0.88 (m, 6H); LCMS (Method H): Rt=1.43 min, m/z=768.3 [M+H]⁺; HPLC (Method G): Rt=8.07 min, 99.30%; Chiral SFC (Method AAA): Rt=2.36 min, 100%.

Isomer 2. ¹H NMR (400 MHz, DMSO-d₆): δ 8.27 (s, 1H), 8.17 (d, J=5.5 Hz, 1H), 7.78 (s, 1H), 7.32-7.06 (m, 3H), 6.65 (d, J=5.4 Hz, 1H), 4.81-4.68 (m, 1H), 4.42-4.04 (m, 5H), 3.78-3.61 (m, 2H), 3.28-3.18 (m, 2H), 3.07-2.60 (m, 11H), 2.43-2.14 (m, 5H), 1.86-1.66 (m, 2H), 1.62-1.30 (m, 13H), 1.18-0.88 (m, 6H); LCMS (Method H): Rt=1.50 min, m/z=768.4 [M+H]⁺; HPLC (Method G): Rt=8.09 min, 92.24%; Chiral SFC (Method AAA): Rt=3.47 min, 100%.

### Step 3. (D1)-5-Fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropyl-N-((2s,4r)-5-azaspiro[3.4]octan-2-yl)benzamide

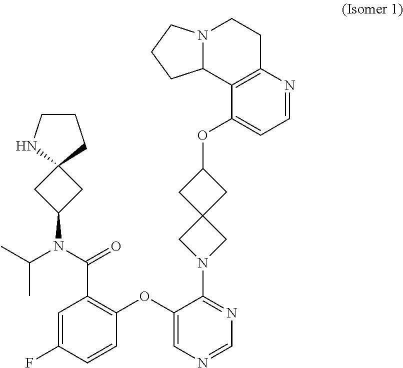 [SMILES: CC(C)N(C(=O)c1cc(F)ccc1Oc1cncnc1N1CC2(CC(Oc3ccnc4c3C3CCCN3CC4)C2)C1)[C@H]1C[C@@]2(CCCN2)C1]

This compound was synthesized following the procedure described for the synthesis of Example 5, step 2, starting with 1 equivalent of (D1)-tert-butyl(2s,4r)-2-(5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamido)-5-azaspiro[3.4]octane-5-carboxylate. After completion, the crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford (D1)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((2s,4r)-5-azaspiro[3.4]octan-2-yl)benzamide as a solid.

Yield: 68.9%; ¹H NMR (400 MHz, DMSO-d₆): δ 8.27 (s, 1H), 8.17 (d, J=5.6 Hz, 1H), 7.79-7.68 (m, 1H), 7.29-7.02 (m, 3H), 6.66 (d, J=5.5 Hz, 1H), 4.74 (quin, J=6.5 Hz, 1H), 4.33-3.90 (m, 5H), 3.77-3.62 (m, 2H), 3.01-2.60 (m, 1 IH), 2.45-2.36 (m, 1H), 2.30-2.17 (m, 2H), 2.13-1.61 (m, 6H), 1.60-1.24 (m, 8H), 1.10-0.91 (m, 3H); LCMS (Method H): Rt=1.17 min, m/z=668.3 [M+H]⁺; HPLC (Method G): Rt=6.22 min, 99.82%; Chiral SFC (Method AAA): Rt=4.22 min, 100%.

### Step 4. (D2)-5-Fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((2s,4r)-5-azaspiro[3.4]octan-2-yl)benzamide (Isomer 2)

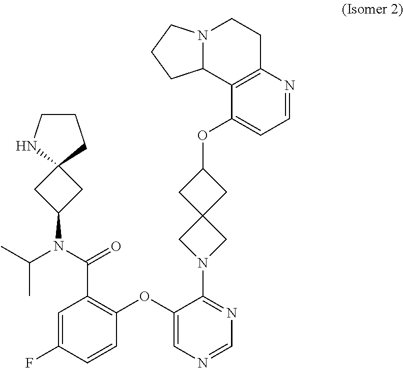 [SMILES: CC(C)N(C(=O)c1cc(F)ccc1Oc1cncnc1N1CC2(CC(Oc3ccnc4c3C3CCCN3CC4)C2)C1)[C@H]1C[C@@]2(CCCN2)C1]

This compound was synthesized following the procedure described for the synthesis of Example 5, step 2, starting with 1 equivalent of (D2)-tert-butyl(2s,4r)-2-(5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamido)-5-azaspiro[3.4]octane-5-carboxylate. After completion, the reaction was concentrated under reduced pressure. The residue was dissolved in saturated sodium bicarbonate solution and the mixture was extracted with 10% MeOH in DCM. The combined organic layer was dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford (D2)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((2s,4r)-5-azaspiro[3.4]octan-2-yl)benzamide as a solid.

Yield: 52.4%; ¹H NMR (400 MHz, DMSO-d₆): δ 8.27 (s, 1H), 8.17 (d, J=5.6 Hz, 1H), 7.83-7.67 (m, 1H), 7.31-7.03 (m, 3H), 6.66 (d, J=5.6 Hz, 1H), 4.74 (quin, J=6.4 Hz, 1H), 4.33-3.91 (m, 5H), 3.75-3.60 (m, 2H), 3.01-2.59 (m, 11H), 2.43-2.36 (m, 1H), 2.30-2.18 (m, 2H), 2.13-1.61 (m, 6H), 1.59-1.25 (m, 8H), 1.14-0.90 (m, 3H); LCMS (Method H): Rt=1.10 min, m/z=668.3 [M+H]⁺; HPLC (Method G): Rt=6.28 min, 99.64%; Chiral SFC (Method N): Rt=1.27 min, 100%.

### Example 146. (D1)-5-Fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((2s,4r)-5-methyl-5-azaspiro[3.4]octan-2-yl)benzamide (Compound No. 146)

### Example 147. (D2)-5-Fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((2s,4r)-5-methyl-5-azaspiro[3.4]octan-2-yl)benzamide (Compound No. 147)

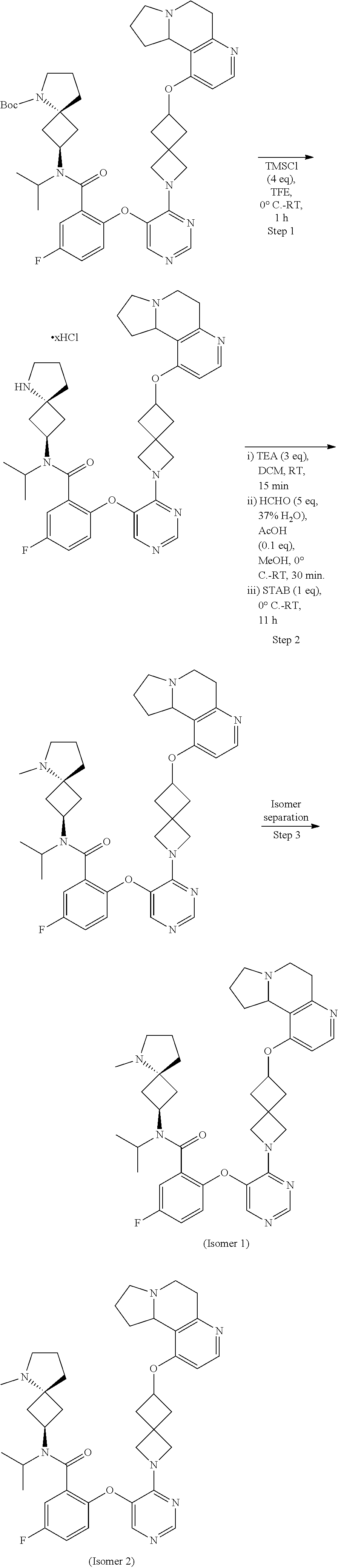

### Step 1. Fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropyl-N-((2s,4r)-5-azaspiro[3.4]octan-2-yl)benzamide xHCl

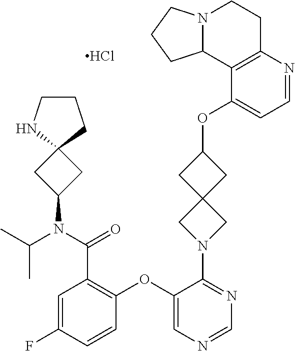 [SMILES: CC(C)N(C(=O)c1cc(F)ccc1Oc1cncnc1N1CC2(CC(Oc3ccnc4c3C3CCCN3CC4)C2)C1)[C@H]1C[C@@]2(CCCN2)C1 ; Cl]

This compound was synthesized following the procedure described for the synthesis of Example 5, step 2, starting with 1 equivalent of tert-butyl (2s,4r)-2-(5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamido)-5-azaspiro[3.4]octane-5-carboxylate. After completion, the reaction was concentrated under reduced pressure to obtain crude 5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((2s,4r)-5-azaspiro[3.4]octan-2-yl)benzamide xHCl as a solid. This material was used without further purification. Yield: Quantitative; LCMS (Method H): Rt=1.11 min, m/z=668.3 [M+H]⁺, 96.65%.

### Step 2. 5-Fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((2s,4r)-5-methyl-5-azaspiro[3.4]octan-2-yl)benzamide

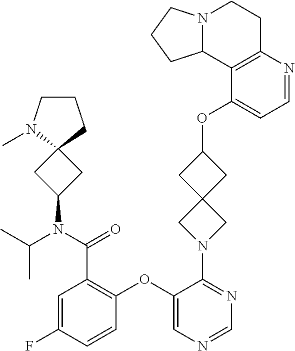 [SMILES: CC(C)N(C(=O)c1cc(F)ccc1Oc1cncnc1N1CC2(CC(Oc3ccnc4c3C3CCCN3CC4)C2)C1)[C@H]1C[C@@]2(CCCN2C)C1]

This compound was synthesized following the procedure described for the synthesis of Example 20, step 4, starting with 1 equivalent of 5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((2s,4r)-5-azaspiro[3.4]octan-2-yl)benzamide xHCl, except that 3 equivalents of TEA, 5 equivalents of formaldehyde (37% in H₂O), 0.1 equivalent of AcOH, and 1 equivalent of STAB were used. The reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford 5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((2s,4r)-5-methyl-5-azaspiro[3.4]octan-2-yl)benzamide as a solid.

Yield: 50.3%; LCMS (Method H): Rt=1.12 min, m/z=682.3 [M+H]⁺; HPLC (Method G): Rt=6.30 min, 99.48%.

### Step 3. (D1)-5-Fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropyl-N-((2s,4r)-5-methyl-5-azaspiro[3.4]octan-2-yl)benzamide and (D2)-5-fluoro-2-((4-(6-((5,6,8,9,10,10α-hexahydropyrrolo[2,1-f][1,6]]naphthyridin-1-yl)oxy)-2-azaspiro[3.3]heptan-2-yl)pyrimidin-5-yl)oxy)-N-isopropyl-N-((2s,4r)-S-methyl-5-azaspiro[3.4]octan-2-yl)benzamide

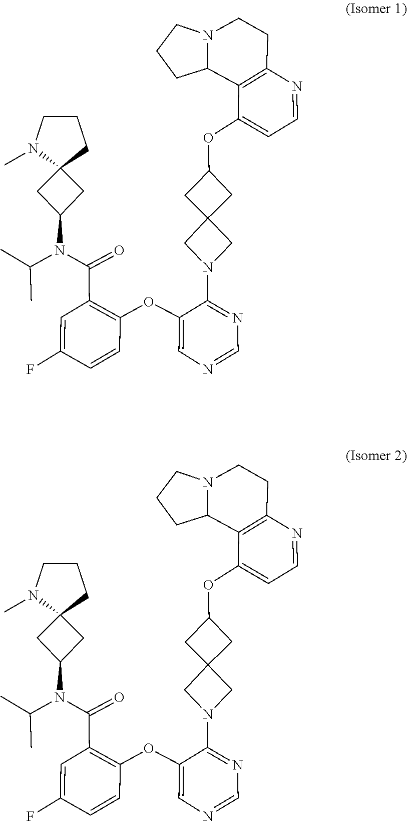 [SMILES: CC(C)N(C(=O)c1cc(F)ccc1Oc1cncnc1N1CC2(CC(Oc3ccnc4c3C3CCCN3CC4)C2)C1)[C@H]1C[C@@]2(CCCN2C)C1 ; CC(C)N(C(=O)c1cc(F)ccc1Oc1cncnc1N1CC2(CC(Oc3ccnc4c3C3CCCN3CC4)C2)C1)[C@H]1C[C@@]2(CCCN2C)C1]

The diastereomeric mixture of 5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((2s,4r)-5-methyl-5-azaspiro[3.4]octan-2-yl)benzamide (193 mg, 0.279 mmol) was purified by chiral Prep SFC (Method S) to obtain (D1)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((2s,4r)-5-methyl-5-azaspiro[3.4]octan-2-yl)benzamide (33.16 mg, 18.47% yield) and (D2)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-((2s,4r)-5-methyl-5-azaspiro[3.4]octan-2-yl)benzamide (45.2 mg, 23.76% yield) as solids.

Isomer 1. ¹H NMR (400 MHz, DMSO-d₆): δ 8.30-8.23 (m, 1H), 8.19-8.14 (m, 1H), 7.79-7.57 (m, 1H), 7.33-7.05 (m, 3H), 6.71-6.59 (m, 1H), 4.75 (quin, J=6.4 Hz, 1H), 4.37-4.10 (m, 4H), 3.94-3.60 (m, 3H), 3.01-2.59 (m, 10H), 2.46-2.36 (m, 2H), 2.33 (s, 3H), 2.29-2.20 (m, 2H), 2.09-1.94 (m, 2H), 1.93-1.67 (m, 4H), 1.59-0.87 (m, 10H); LCMS (Method H): Rt=1.10 min, m/z=682.3 [M+H]⁺; HPLC (Method G): Rt=6.32 min, 99.83%; Chiral SFC (Method AAA): Rt=4.76 min, 100%. Isomer 2. ¹H NMR (400 MHz, DMSO-d₆): δ 8.32-8.21 (m, 1H), 8.16 (br d, J=5.4 Hz, 1H), 7.80-7.56 (m, 1H), 7.35-7.02 (m, 3H), 6.71-6.58 (m, 1H), 4.75 (quin, J=6.4 Hz, 1H), 4.35-4.11 (m, 4H), 3.96-3.61 (m, 3H), 3.01-2.61 (m, 10H), 2.45-2.35 (m, 2H), 2.33 (s, 3H), 2.30-2.18 (m, 2H), 2.06-1.96 (m, 2H), 1.93-1.66 (m, 4H), 1.57-0.91 (m, 10H); LCMS (Method H): Rt=1.10 min, m/z=682.3 [M+H]⁺; HPLC (Method G): Rt=6.32 min, 99.81%; Chiral SFC (Method AAC): Rt=7.79 min, 100%.

### Example 148. (D1)-N-((1r,3r)-3-(Dimethylamino)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (Compound No. 148)

### Example 149. (D2)-N-((1r,3r)-3-(Dimethylamino)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (Compound No. 149)

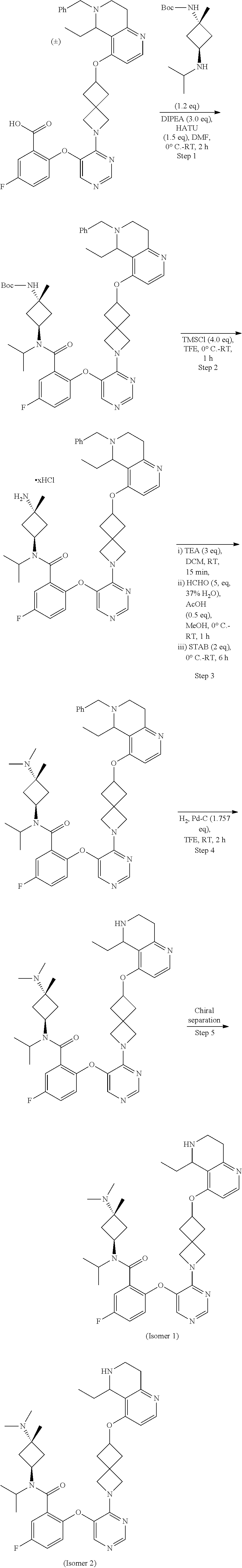

### Step 1. tert-Butyl ((1r,3r)-3-(2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-1-methylcyclobutyl)carbamate

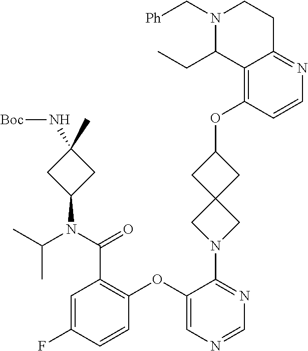 [SMILES: CCC1c2c(OC3CC4(C3)CN(c3ncncc3Oc3ccc(F)cc3C(=O)N(C(C)C)[C@H]3C[C@](C)(NC(=O)OC(C)(C)C)C3)C4)ccnc2CCN1Cc1ccccc1]

This compound was synthesized following the procedure described for the synthesis of Example 1, step 2, starting with 1 equivalent of (±)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.2 equivalents of tert-butyl ((1r,3r)-3-(isopropylamino)-1-methylcyclobutyl)carbamate. After work-up, the crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure to obtain tert-butyl ((1r,3r)-3-(2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-1-methylcyclobutyl)carbamate as a solid.

Yield: 49.7%; LCMS (Method A): Rt=2.77 min, m/z=820.2 [M+H]⁺, 95.40%.

### Step 2. N-((1r,3r)-3-Amino-3-methylcyclobutyl)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl

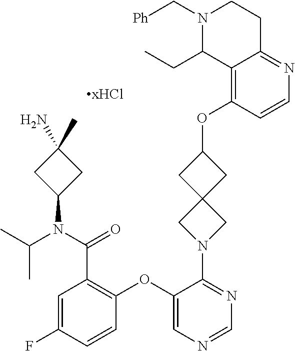 [SMILES: CCC1c2c(OC3CC4(C3)CN(c3ncncc3Oc3ccc(F)cc3C(=O)N(C(C)C)[C@H]3C[C@](C)(N)C3)C4)ccnc2CCN1Cc1ccccc1]

This compound was synthesized following the procedure described for the synthesis of Example 20, step 3, starting with 1 equivalent of tert-butyl ((1r,3r)-3-(2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-1-methylcyclobutyl)carbamate. After completion, the reaction was concentrated under reduced pressure to obtain crude N-((1r,3r)-3-amino-3-methylcyclobutyl)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl as a solid. This material was used without further purification.

Yield: 91%; LCMS (Method A): Rt=2.22 min, m/z=720.2 [M+H]⁺, 82.90%.

### Step 3. 2-((4-(6-((6-Benzyl-5-ethyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide

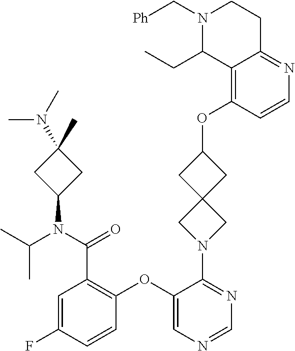 [SMILES: CCC1c2c(OC3CC4(C3)CN(c3ncncc3Oc3ccc(F)cc3C(=O)N(C(C)C)[C@H]3C[C@](C)(N(C)C)C3)C4)ccnc2CCN1Cc1ccccc1]

This compound was synthesized following the procedure described for the synthesis of Example 20, step 4, starting with 1 equivalent of N-((1r,3r)-3-amino-3-methylcyclobutyl)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide xHCl, except that 5 equivalents of formaldehyde (37% in H₂O), 3 equivalents of TEA, 0.5 equivalent of AcOH, and 2 equivalents of STAB were used. After completion, the reaction was concentrated under reduced pressure. 10% NaHCO₃ solution was added to this crude material, and the mixture was extracted with DCM. The combined organic layer was washed with brine solution, dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure to obtain 2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide as a solid.

Yield: 77%; LCMS (Method B): Rt=1.32 min, m/z=748.5 [M+H]⁺; HPLC (Method C): Rt=12.10 min, 96.13%.

### Step 4. N-((1r,3r)-3-(Dimethylamino)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide

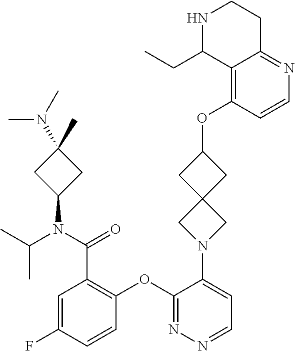 [SMILES: CCC1NCCc2nccc(OC3CC4(C3)CN(c3ccnnc3Oc3ccc(F)cc3C(=O)N(C(C)C)[C@H]3C[C@](C)(N(C)C)C3)C4)c21]

This compound was synthesized following the procedure described for the synthesis of Example 20, step 5, starting with 1 equivalent of 2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide, except that 1.757 equivalents of Pd—C was used. After completion, the reaction was filtered through a Celite® pad, and the pad was washed with MeOH. The filtrate was concentrated under reduced pressure to obtain crude N-((1r,3r)-3-(dimethylamino)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide as a solid. This material was used without further purification.

Yield: 93%; LCMS (Method A): Rt=1.88 min, m/z=658.5 [M+H]⁺, 91.58%; Chiral SFC (Method AU)=Peak 1: Rt=4.18 min, 49.45%; Peak 2: Rt=5.54 min, 48.95%.

### Step 5. (D1)-N-((1r,3r)-3-(Dimethylamino)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide and (D2)-N-((1r,3r)-3-(dimethylamino)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide

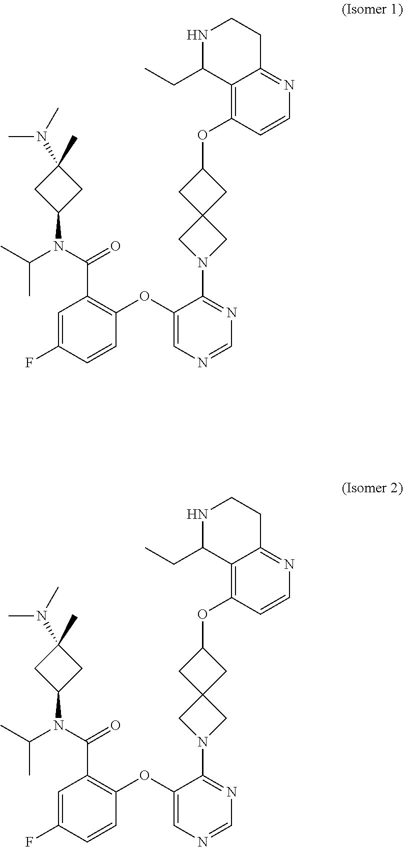 [SMILES: CCC1NCCc2nccc(OC3CC4(C3)CN(c3ncncc3Oc3ccc(F)cc3C(=O)N(C(C)C)[C@H]3C[C@](C)(N(C)C)C3)C4)c21 ; CCC1NCCc2nccc(OC3CC4(C3)CN(c3ncncc3Oc3ccc(F)cc3C(=O)N(C(C)C)[C@H]3C[C@](C)(N(C)C)C3)C4)c21]

The diastereomeric mixture of N-((1r,3r)-3-(dimethylamino)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (250 mg, 0.380 mmol) was purified by chiral Prep SFC (Method L) obtain (D1)-N-((1r,3r)-3-(dimethylamino)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (66 mg, 25.5% yield) and (D2)-N-((1r,3r)-3-(dimethylamino)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (68 mg, 26.4% yield) as solids.

Isomer 1. ¹H NMR (400 MHz, DMSO-d₆): δ 8.32-8.23 (m, 1H), 8.14 (br d, J=5.5 Hz, 1H), 7.78-7.58 (m, 1H), 7.30-7.03 (m, 3H), 6.68-6.58 (m, 1H), 4.79-4.66 (m, 1H), 4.42-4.10 (m, 4H), 4.04-3.60 (m, 3H), 3.05-2.74 (m, 5H), 2.67-2.57 (m, 2H), 2.25-2.17 (m, 2H), 2.16-1.87 (m, 8H), 1.80-1.47 (m, 3H), 1.46-1.23 (m, 3H), 1.14-0.89 (m, 10H); LCMS (Method C): Rt=1.41 min, m/z=658.4 [M+H]⁺; HPLC (Method C): Rt=8.36 min, 96.66%; Chiral SFC (Method AU)=Peak 1: Rt=4.92 min, 99.41%.

Isomer 2. ¹H NMR (400 MHz, DMSO-d₆): δ 8.32-8.21 (m, 1H), 8.15 (d, J=5.4 Hz, 11H), 7.77-7.59 (m, 1H), 7.31-7.06 (m, 3H), 6.67-6.59 (m, 1H), 4.78-4.68 (m, 1H), 4.37-4.10 (m, 4H), 4.05-3.62 (m, 3H), 3.05-2.75 (m, 5H), 2.66-2.58 (m, 2H), 2.27-2.16 (m, 2H), 2.13-1.87 (m, 8H), 1.81-1.47 (m, 3H), 1.46-1.27 (m, 3H), 1.15-0.89 (m, 10H); LCMS (Method C): Rt=1.41 min, m/z=658.4 [M+H]⁺; HPLC (Method C): Rt=8.35 min, 97.16%; Chiral SFC (Method AU)=Peak 1: Rt=6.54 min, 97.27%.

### Example 150. (D1)-N-((1r,3r)-3-(Azetidin-1-yl)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (Compound No. 150)

### Example 151. (D2)-N-((1r,3r)-3-(Azetidin-1-yl)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (Compound No. 151)

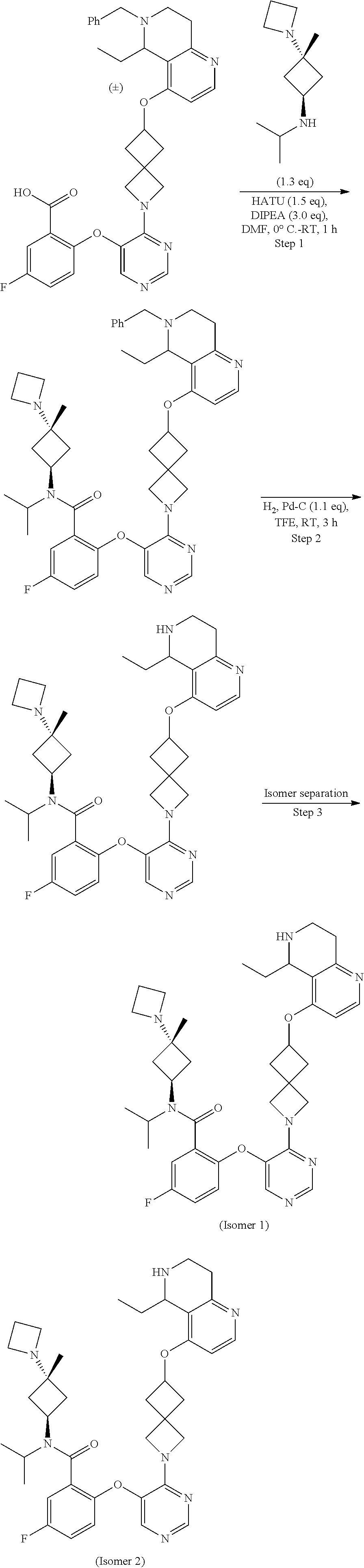

### Step 1. N-((1r,3r)-3-(Azetidin-1-yl)-3-methylcyclobutyl)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide

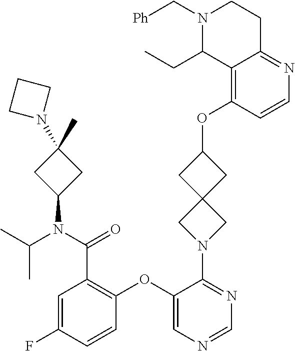 [SMILES: CCC1c2c(OC3CC4(C3)CN(c3ncncc3Oc3ccc(F)cc3C(=O)N(C(C)C)[C@H]3C[C@](C)(N5CCC5)C3)C4)ccnc2CCN1Cc1ccccc1]

This compound was synthesized following the procedure described for the synthesis of Example 1, Step 2, starting with 1 equivalent of (±)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluorobenzoic acid (1.3 g, 2.182 mmol) and 1.3 equivalents of (1r,3r)-3-(azetidin-1-yl)-N-isopropyl-3-methylcyclobutan-1-amine. After work-up, the crude compound was purified by reverse phase chromatography using a SiliCycle SiliaSep C18 column (120 g) on Biotage isolera one (Mobile phase A: 10 mM NH₄HCO₃ in water, B: ACN), then by achiral Prep SFC (Method B). The pure fractions were concentrated under reduced pressure and lyophilized to obtain

N-((1r,3r)-3-(azetidin-1-yl)-3-methylcyclobutyl)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide as a solid.

Yield: 56.8%; ¹H NMR (400 MHz, DMSO-d₆): δ 8.31-8.25 (m, 1H), 8.20 (d, J=5.5 Hz, 1H), 7.81-7.57 (m, 1H), 7.38-7.04 (m, 8H), 6.73-6.63 (m, 1H), 4.78-4.68 (m, 1H), 4.39-4.10 (m, 4H), 3.79-3.63 (m, 3H), 3.62-3.50 (m, 2H), 3.22-3.07 (m, 3H), 2.97-2.87 (m, 2H), 2.83-2.69 (m, 4H), 2.08 (s, 4H), 1.93-1.71 (m, 3H), 1.66-1.52 (m, 2H), 1.47-0.83 (m, 12H), two protons merged with solvent peaks; LCMS (Method A): Rt=2.32 min, m/z=760.3 [M+H]⁺; HPLC (Method C): Rt=11.62 min, 97.80%.

### Step 2. N-((1r,3r)-3-(Azetidin-1-yl)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide

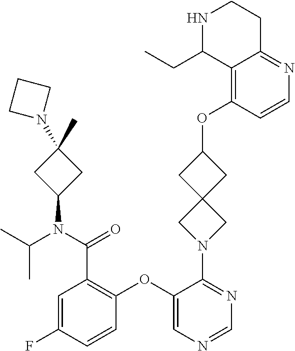 [SMILES: CCC1NCCc2nccc(OC3CC4(C3)CN(c3ncncc3Oc3ccc(F)cc3C(=O)N(C(C)C)[C@H]3C[C@](C)(N5CCC5)C3)C4)c21]

The reaction was performed following the same procedure in two batches.

This compound was synthesized following the procedure described for the synthesis of Example 20, step 5, starting with 1 equivalent of N-((1r,3r)-3-(azetidin-1-yl)-3-methylcyclobutyl)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide, except that 1.1 equivalents of Pd—C was used. After completion, the reaction was filtered through a Celite® pad, and the pad was washed with TFE. The filtrate was concentrated under reduced pressure. After concentration, the crude from both batches were combined to afford crude N-((1r,3r)-3-(azetidin-1-yl)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide as a solid. Combined Yield: 81.9%; LCMS (Method H): Rt=1.05 min, m/z=670.3 [M+H]⁺, 95.30%; Chiral SFC (Method AAE): Peak 1: Rt=5.13 min, 46.70%; Peak 2: Rt=6.71 min, 48.59%.

### Step 3. (D1)-N-((1r,3r)-3-(Azetidin-1-yl)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide and (D2)-N-((1r,3r)-3-(azetidin-1-yl)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide

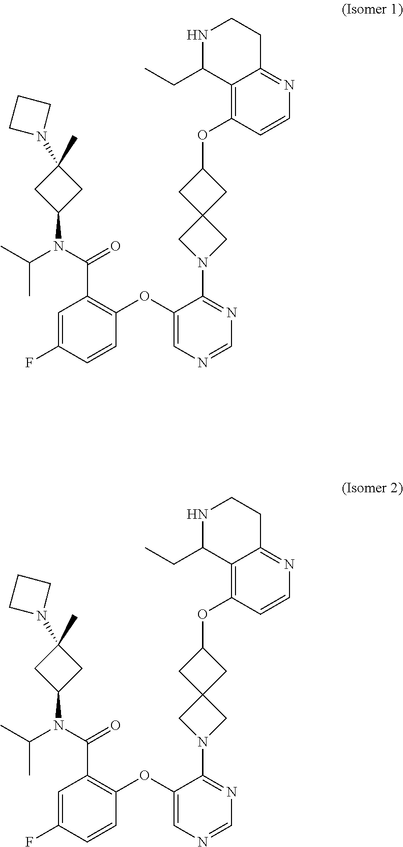 [SMILES: CCC1NCCc2nccc(OC3CC4(C3)CN(c3ncncc3Oc3ccc(F)cc3C(=O)N(C(C)C)[C@H]3C[C@](C)(N5CCC5)C3)C4)c21 ; CCC1NCCc2nccc(OC3CC4(C3)CN(c3ncncc3Oc3ccc(F)cc3C(=O)N(C(C)C)[C@H]3C[C@](C)(N5CCC5)C3)C4)c21]

The diastereomeric mixture of N-((1r,3r)-3-(azetidin-1-yl)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (490 mg, 0.73 mmol) was purified by chiral Prep SFC (Method U) to obtain both isomers (D1)-N-((1r,3r)-3-(azetidin-1-yl)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (145 mg, 28.4% yield) and (D2)-N-((1r,3r)-3-(azetidin-1-yl)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (140 mg, 27.5% yield) as solids.

Isomer 1. ¹H NMR (400 MHz, DMSO-d₆): δ 8.33-8.24 (m, 1H), 8.15 (d, J=5.5 Hz, 11H), 7.79-7.58 (m, 1H), 7.38-7.04 (m, 3H), 6.69-6.58 (m, 1H), 4.81-4.69 (m, 1H), 4.43-4.09 (m, 4H), 3.83-3.63 (m, 3H), 3.18-3.07 (m, 2H), 3.05-2.86 (m, 4H), 2.85-2.72 (m, 3H), 2.70-2.58 (m, 2H), 2.28-1.97 (m, 4H), 1.91-1.74 (m, 3H), 1.67-1.50 (m, 2H), 1.46-0.86 (m, 12H), one proton merged with solvent peaks; LCMS (Method H): Rt=1.04 min, m/z=670.3 [M+H]⁺; HPLC (Method G): Rt=6.65 min, 96.73%; Chiral SFC (Method AAE): Rt=4.77 min, 100%.

Isomer 2. ¹H NMR (400 MHz, DMSO-d₆): δ 8.32-8.25 (m, 1H), 8.15 (d, J=5.5 Hz, 1H), 7.81-7.58 (m, 1H), 7.34-7.06 (m, 3H), 6.70-6.58 (m, 1H), 4.78-4.66 (m, 1H), 4.40-4.09 (m, 4H), 3.83-3.62 (m, 3H), 3.17-3.08 (m, 2H), 3.05-2.85 (m, 4H), 2.85-2.72 (m, 3H), 2.70-2.61 (m, 2H), 2.28-1.97 (m, 4H), 1.95-1.73 (m, 3H), 1.66-1.48 (m, 2H), 1.46-0.86 (m, 12H), one proton merged with solvent peaks; LCMS (Method H): Rt=1.05 min, m/z=670.4 [M+H]⁺; HPLC (Method G): Rt=6.30 min, 97.36%; Chiral SFC (Method AAE): Rt=6.17 min, 99.52%.

### Example 152. (D1)-2-((4-(6-((5-Ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide (Compound No. 152)

### Example 153. (D2)-2-((4-(6-((5-Ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide (Compound No. 153)

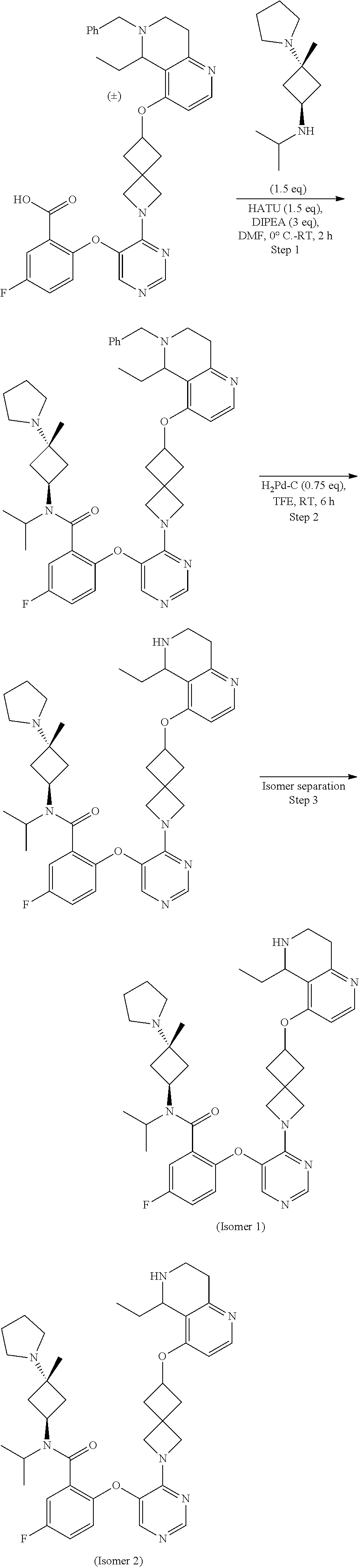

### Step 1. 2-((4-(6-((6-Benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide

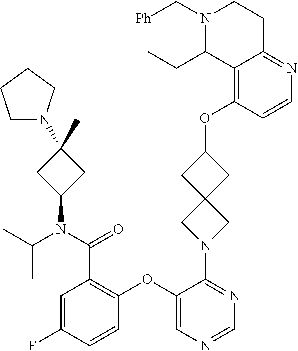 [SMILES: CCC1c2c(OC3CC4(C3)CN(c3ncncc3Oc3ccc(F)cc3C(=O)N(C(C)C)[C@H]3C[C@](C)(N5CCCC5)C3)C4)ccnc2CCN1Cc1ccccc1]

This compound was synthesized following the procedure described for the synthesis of Example 1, step 2, starting with 1 equivalent of (±)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.5 equivalents of (1r,3r)—N-isopropyl-3-methyl-3-(pyrrolidin-1-yl)cyclobutan-1-amine. After work-up, the crude compound was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure to afford 2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide as a solid.

Yield: 44.5%; ¹H NMR (400 MHz, DMSO-d₆): δ 8.30-8.26 (m, 1H), 8.20 (d, J=5.5 Hz, 11H), 7.78-7.56 (m, 11H), 7.36-7.21 (m, 7H), 7.18-7.05 (m, 1H), 6.72-6.63 (m, 1H), 4.73 (quin, J=6.4 Hz, 1H), 4.36-4.08 (m, 4H), 3.77-3.64 (m, 2H), 3.62-3.50 (m, 2H), 3.22-3.11 (m, 1H), 2.99-2.72 (m, 5H), 2.68-2.57 (m, 2H), 2.48-2.33 (m, 2H), 2.29-2.09 (m, 4H), 1.97-1.77 (m, 1H), 1.73-1.50 (m, 6H), 1.47-0.84 (m, 12H), two protons merged with solvent peaks; LCMS (Method B): Rt=1.34 min, m/z=774.6 [M+H]⁺, 96.33%.

### Step 2. 2-((4-(6-((5-Ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide

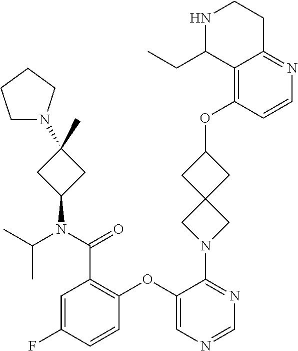 [SMILES: CCC1NCCc2nccc(OC3CC4(C3)CN(c3ncncc3Oc3ccc(F)cc3C(=O)N(C(C)C)[C@H]3C[C@](C)(N5CCCC5)C3)C4)c21]

This compound was synthesized following the procedure described for the synthesis of Example 20, step 5, starting with 1 equivalent of 2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide, except that 0.75 equivalent of Pd—C was used. After completion, the reaction was filtered through a Celite® pad, and the pad was washed with EtOAc:THF (1:1). The filtrate was concentrated under reduced pressure to afford crude 2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide as a solid. This material was used without further purification.

Yield: 76%; LCMS (Method H): Rt=1.07 min, m/z=684.3 [M+H]⁺, 99.82%; Chiral SFC (Method AZ): Peak 1: Rt=4.36 min, 44.32%; Peak 2: 5.50 min, 45.96%.

### Step 3. (D1)-2-((4-(6-((5-Ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide and (D2)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide

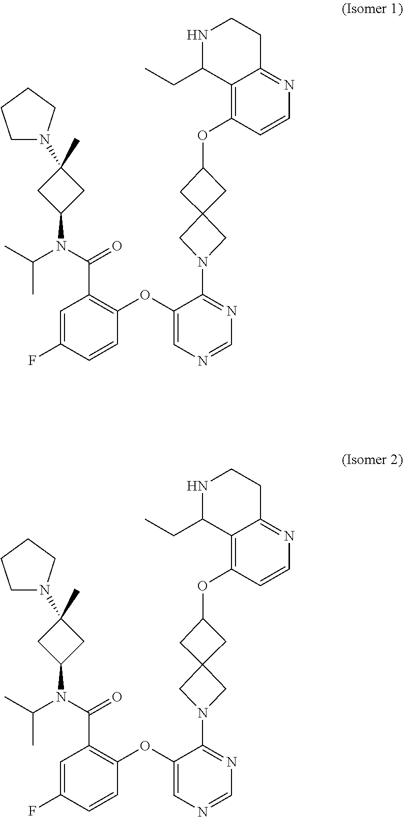 [SMILES: CCC1NCCc2nccc(OC3CC4(C3)CN(c3ncncc3Oc3ccc(F)cc3C(=O)N(C(C)C)[C@H]3C[C@](C)(N5CCCC5)C3)C4)c21 ; CCC1NCCc2nccc(OC3CC4(C3)CN(c3ncncc3Oc3ccc(F)cc3C(=O)N(C(C)C)[C@H]3C[C@](C)(N5CCCC5)C3)C4)c21]

The diastereomeric mixture of 2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide (540 mg, 0.79 mmol) was purified by chiral Prep SFC (Method V) to obtain (D1)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide (120 mg, 22.02% yield) and (D2)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide (125 mg, 23.08% yield) as solids.

Isomer 1. ¹H NMR (400 MHz, DMSO-d₆): δ 8.34-8.25 (m, 1H), 8.14 (br d, J=5.5 Hz, 1H), 7.80-7.56 (m, 1H), 7.36-7.02 (m, 3H), 6.69-6.58 (m, 1H), 4.78-4.67 (m, 1H), 4.38-4.01 (m, 5H), 3.90-3.63 (m, 3H), 3.07-2.95 (m, 1H), 2.93-2.76 (m, 4H), 2.70-2.61 (m, 2H), 2.42-2.35 (m, 2H), 2.28-2.07 (m, 4H), 2.00-1.77 (m, 2H), 1.73-1.49 (m, 6H), 1.46-0.88 (m, 12H), one proton merged with solvent peaks; LCMS (Method H): Rt=1.06 min, m/z=684.3 [M+H]⁺; HPLC (Method G): Rt=6.48 min, 99.11%; Chiral SFC (Method AZ): Rt=4.23 min, 96.96%.

Isomer 2. ¹H NMR (400 MHz, DMSO-d₆): δ 8.32-8.25 (m, 1H), 8.15 (br d, J=5.4 Hz, 1H), 7.80-7.57 (m, 1H), 7.33-7.04 (m, 3H), 6.66-6.58 (m, 1H), 4.82-4.64 (m, 1H), 4.41-3.99 (m, 5H), 3.91-3.65 (m, 3H), 3.08-2.95 (m, 1H), 2.94-2.75 (m, 4H), 2.70-2.61 (m, 2H), 2.41-2.34 (m, 2H), 2.29-2.07 (m, 4H), 2.01-1.74 (m, 2H), 1.72-1.49 (m, 6H), 1.47-0.84 (m, 12H), one proton merged with solvent peaks; LCMS (Method H): Rt=1.06 min, m/z=684.4 [M+H]⁺; HPLC (Method G): Rt=6.48 min, 99.72%; Chiral SFC (Method AZ): Rt=5.44 min, 98.68%.

### Example 154. (D1)-2-((4-(6-((5-Ethyl-5,6,7,8-tetrahydro-1,6-naphthyridin-4-yl)oxy)-2-azaspiro[3.3]heptan-2-yl)pyrimidin-5-yl)oxy-5-fluoro-N-isopropyl-N-((2s,4r)-5-methyl-5-azaspiro[3.4]octan-2-yl)benzamide (Compound No. 154)

### Example 155. (D2)-2-((4-(6-((5-Ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide (Compound No. 155)

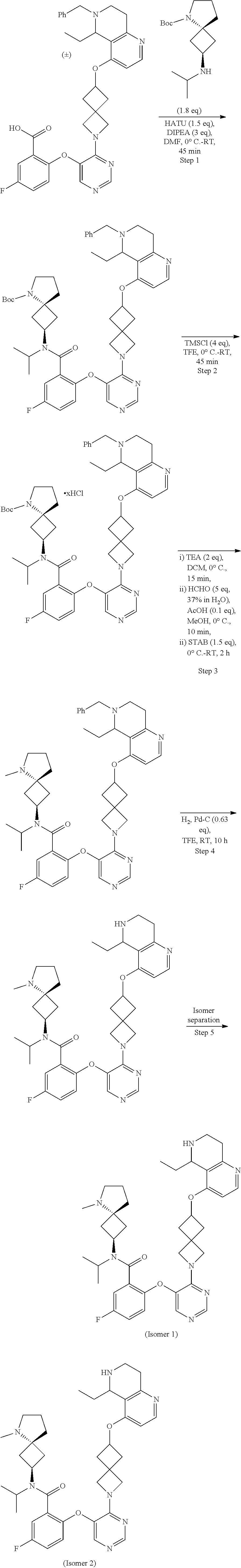

### Step 1. tert-Butyl(2s,4r)-2-(2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.4]octane-5-carboxylate

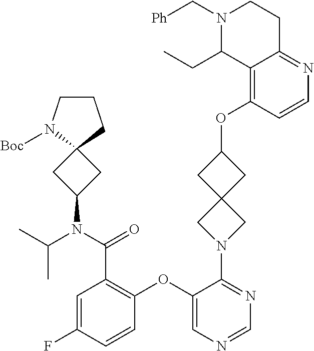 [SMILES: CCC1c2c(OC3CC4(C3)CN(c3ncncc3Oc3ccc(F)cc3C(=O)N(C(C)C)[C@H]3C[C@@]5(CCCN5C(=O)OC(C)(C)C)C3)C4)ccnc2CCN1Cc1ccccc1]

The reaction was performed following the same procedure in two batches.

This compound was synthesized following the procedure described for the synthesis of Example 1, Step 2, starting with 1 equivalent of (f)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.8 equivalents of tert-butyl(2s,4r)-2-(isopropylamino)-5-azaspiro[3.4]octane-5-carboxylate. After completion, the reaction mixture from both batches were combined for work-up. The crude was purified by reverse phase chromatography using a SiliCycle SiliaSep C18 column (120 g) on Biotage isolera one (Mobile phase A: 10 mM NH₄HCO₃ in water, B: ACN) to obtain tert-butyl(2s,4r)-2-(2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.4]octane-5-carboxylate as a solid.

Combined yield: 73.9%; ¹H NMR (400 MHz, DMSO-d₆): δ 8.27 (s, 1H), 8.20 (d, J=5.5 Hz, 1H), 7.77 (s, 1H), 7.38-7.00 (m, 8H), 6.67 (br d, J=5.4 Hz, 1H), 4.79-4.66 (m, 1H), 4.42-4.02 (m, 5H), 3.79-3.63 (m, 2H), 3.62-3.48 (m, 2H), 3.27-3.12 (m, 3H), 3.08-2.99 (m, 1H), 2.97-2.84 (m, 1H), 2.83-2.69 (m, 4H), 2.32-1.88 (m, 5H), 1.67-1.31 (m, 14H), 1.22-0.78 (m, 8H), two protons merged with solvent peaks; LCMS (Method A): Rt=2.72 min, m/z=846.3 [M+H]⁺, 96.43%.

### Step 2. 2-((4-(6-((6-Benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-azaspiro[3.4]octan-2-yl)benzamide xHCl

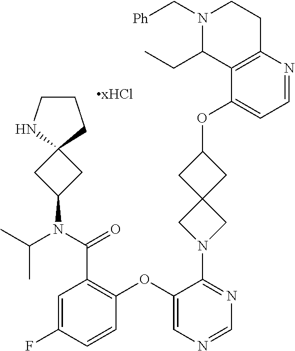 [SMILES: CCC1c2c(OC3CC4(C3)CN(c3ncncc3Oc3ccc(F)cc3C(=O)N(C(C)C)[C@H]3C[C@@]5(CCCN5)C3)C4)ccnc2CCN1Cc1ccccc1 ; "·xHCl"]

This compound was synthesized following the procedure described for the synthesis of Example 20, step 3, starting with 1 equivalent of tert-butyl(2s,4r)-2-(2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.4]octane-5-carboxylate. After completion, the reaction was concentrated under reduced pressure and the residue was triturated with EtOAc. The organic layer was decanted and the solid was dried under vacuum to obtain crude 2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-azaspiro[3.4]octan-2-yl)benzamide xHCl. This material was used without further purification.

Quantitative yield; LCMS (Method A): Rt=2.30 min, m/z=746.3 [M+H]⁺, 87.46%.

### Step 3. 2-((4-(6-((6-Benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide

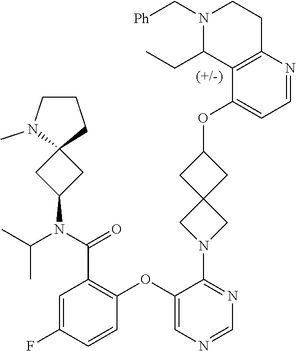 [SMILES: CCC1c2c(OC3CC4(C3)CN(c3ncncc3Oc3ccc(F)cc3C(=O)N(C(C)C)[C@H]3C[C@@]5(CCCN5C)C3)C4)ccnc2CCN1Cc1ccccc1]

The reaction was performed following the same procedure in two batches.

This compound was synthesized following the procedure described for the synthesis of Example 20, Step 4, starting with 1 equivalent of 2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-azaspiro[3.4]octan-2-yl)benzamide xHCl, except that 5 equivalents of formaldehyde (37% in H₂O), 0.1 equivalent of AcOH, and 1.5 equivalents of STAB were used. The reaction mixtures from both batches were combined for work-up, and the crude was purified by reverse phase chromatography using a SiliCycle SiliaSep C18 column (120 g) on Biotage isolera one (Mobile phase A: 10 mM NH₄HCO₃ in water, B: ACN) to obtain 2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide as a solid.

Combined Yield: 60.13%; ¹H NMR (400 MHz, DMSO-d₆): δ 8.30-8.24 (m, 1H), 8.20 (br d, J=5.4 Hz, 1H), 7.80-7.56 (m, 1H), 7.42-7.05 (m, 8H), 6.75-6.64 (m, 1H), 4.81-4.65 (m, 1H), 4.41-4.05 (m, 4H), 3.77-3.49 (m, 5H), 3.25-3.12 (m, 1H), 3.03-2.70 (m, 5H), 2.46-2.38 (m, 2H), 2.33 (br s, 2H), 2.28-2.10 (m, 3H), 2.05-1.85 (m, 3H), 1.78-1.68 (m, 1H), 1.65-1.48 (m, 4H), 1.45 (br d, J=5.9 Hz, 9H); two protons merged with solvent peaks; LCMS (Method A): Rt=2.40 min, m/z=760.3 [M+H]⁺, 93.33%.

### Step 4. 2-((4-(6-((5-Ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide

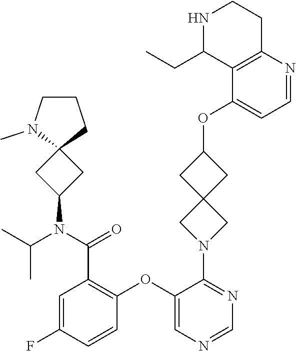 [SMILES: CCC1NCCc2nccc(OC3CC4(C3)CN(c3ncncc3Oc3ccc(F)cc3C(=O)N(C(C)C)[C@H]3C[C@@]5(CCCN5C)C3)C4)c21]

This compound was synthesized following the procedure described for the synthesis of Example 20, step 5, starting with 1 equivalent of 2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide, except that 0.63 equivalent of Pd—C was used. After filtration, the filtrate was concentrated under reduced pressure to obtain crude 2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide as a solid. This material was used without further purification.

Yield: 91%; LCMS (Method B): Rt=0.84 min, m/z=670.3 [M+H]⁺, 99.54%; Chiral SFC (Method AAA): Peak 1: Rt=2.64 min, 48.66%; Peak 2: Rt=3.45 min, 50.70%.

### Step 5. (D1)-2-((4-(6-((5-Ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide and (D2)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide

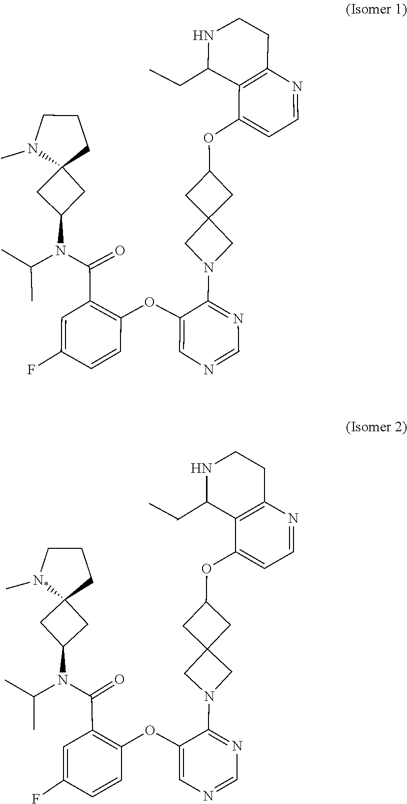

The diastereomeric mixture of 2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide (510 mg, 0.76 mmol) was purified by chiral Prep SFC (Method W) to obtain (D1)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide (183 mg, 35.9% yield) and (D2)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide (180 mg, 35.3% yield) as solids.

Isomer 1. ¹H NMR (400 MHz, DMSO-d₆): δ 8.31-8.23 (m, 1H), 8.15 (br d, J=5.5 Hz, 1H), 7.80-7.56 (m, 1H), 7.35-7.04 (m, 3H), 6.67-6.57 (m, 1H), 4.80-4.64 (m, 1H), 4.43-4.10 (m, 4H), 3.80-3.63 (m, 3H), 3.06-2.86 (m, 3H), 2.84-2.62 (m, 5H), 2.48-2.38 (m, 1H), 2.33 (s, 3H), 2.27-2.14 (m, 3H), 2.09-1.87 (br t, J=7.9 Hz, 4H), 1.76-1.49 (m, 4H), 1.47-0.88 (m, 9H), one proton merged with solvent peaks; LCMS (Method H): Rt=1.12 min, m/z=670.3 [M+H]⁺; HPLC (Method G): Rt=6.32 min, 99.58%; Chiral SFC (Method AAA): Rt=3.37 min, 100%.

Isomer 2. ¹H NMR (400 MHz, DMSO-d₆): δ 8.31-8.23 (m, 1H), 8.15 (br d, J=5.4 Hz, 1H), 7.80-7.54 (m, 1H), 7.34-7.06 (m, 3H), 6.68-6.59 (m, 1H), 4.80-4.68 (m, 1H), 4.41-4.09 (m, 4H), 3.82-3.62 (m, 3H), 3.05-2.86 (m, 3H), 2.84-2.61 (m, 5H), 2.46-2.41 (m, 1H), 2.33 (s, 3H), 2.28-2.15 (m, 3H), 2.07-1.87 (m, 4H), 1.77-1.49 (m, 4H), 1.47-0.89 (m, 9H), one proton merged with solvent peaks; LCMS (Method H): Rt=1.12 min, m/z=670.3 [M+H]⁺; HPLC (Method G): Rt=6.33 min, 99.11%; Chiral SFC (Method AAA): Rt=4.82 min, 99.30%.

### Example 156. (D1)-N-((1r,3r)-3-(Azetidin-1-yl)-3-methylcyclobutyl)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide (Compound No. 156)

### Example 157. (D2)-N-((1r,3r)-3-(Azetidin-1-yl)-3-methylcyclobutyl)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide (Compound No. 157)

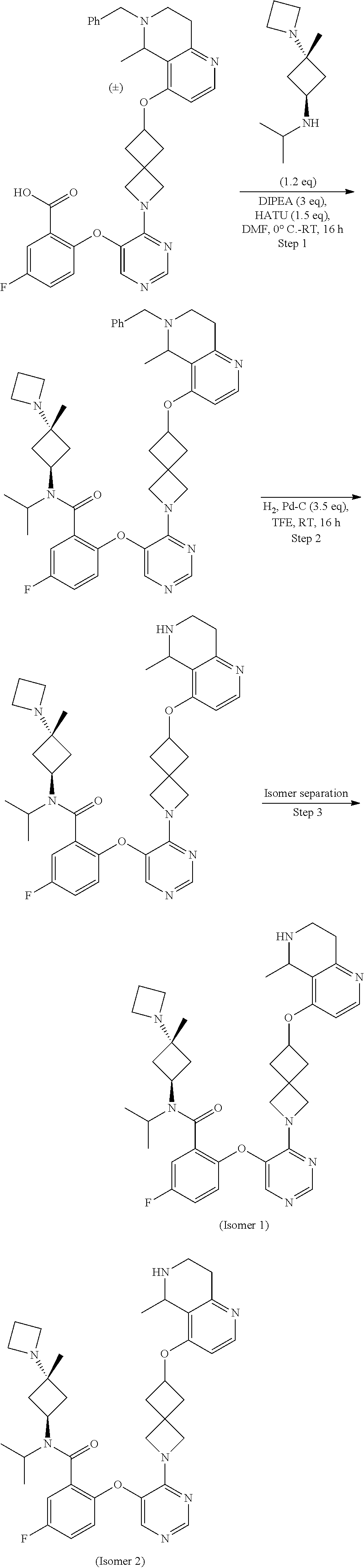

### Step 1. N-((1r,3r)-3-(Azetidin-1-yl)-3-methylcyclobutyl)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide

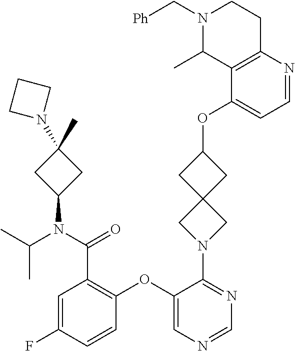 [SMILES: CC1c2c(OC3CC4(C3)CN(c3ncncc3Oc3ccc(F)cc3C(=O)N(C(C)C)[C@H]3C[C@](C)(N5CCC5)C3)C4)ccnc2CCN1Cc1ccccc1]

The reaction was performed following the same procedure in two batches.

This compound was synthesized following the procedure described for the synthesis of Example 1, step 2, starting with 1 equivalent of (±)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.2 equivalents of (1r,3r)-3-(azetidin-1-yl)-N-isopropyl-3-methylcyclobutan-1-amine. After completion, the reaction mixtures from both batches were combined and concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure to afford N-((1r,3r)-3-(azetidin-1-yl)-3-methylcyclobutyl)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide as a solid.

Combined yield: 64.76%; LCMS (Method A): Rt=2.15 min, m/z=746.4 [M+H]⁺; HPLC (Method C): Rt=10.61 min, 91.46%.

### Step 2. N-((1r,3r)-3-(Azetidin-1-yl)-3-methylcyclobutyl)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide

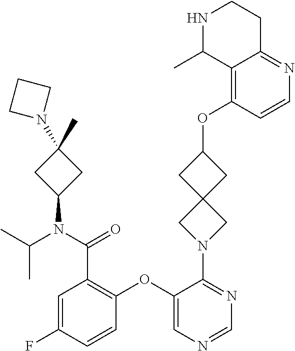 [SMILES: CC1NCCc2nccc(OC3CC4(C3)CN(c3ncncc3Oc3ccc(F)cc3C(=O)N(C(C)C)[C@H]3C[C@](C)(N5CCC5)C3)C4)c21]

The reaction was performed following the same procedure in two batches.

This compound was synthesized following the procedure described for the synthesis of Example 20, step 5, starting with 1 equivalent of N-((1r,3r)-3-(azetidin-1-yl)-3-methylcyclobutyl)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide, except that 3.5 equivalents of Pd—C was used. After completion, the reaction mixtures from both batches were combined and filtered through a Celite® pad, and the pad was washed with MeOH. The filtrate was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN), then by achiral Prep SFC (Method A) to obtain N-((1r,3r)-3-(azetidin-1-yl)-3-methylcyclobutyl)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 25.27%; LCMS (Method H): Rt=1.08 min, m/z=656.3 [M+H]⁺; HPLC (Method C): Rt=7.86 min, 91.83%; Chiral SFC (Method AZ): Peak 1: Rt=3.91 min, 47.90%; Peak 2: Rt=4.74 min, 48.55%.

### Step 3. (D1)-N-((1r,3r)-3-(Azetidin-1-yl)-3-methylcyclobutyl)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide and (±)-2)-N-((1r,3r)-3-(azetidin-1-yl)-3-methylcyclobutyl)-5-fluoro-N-isopropyl-2-((4-(6-((S-methyl-5,6,7,8-tetrahydro-1,6-naphthyridin-4-yl)oxy)-2-azaspiro[3.3]heptan-2-yl)pyrimidin-5-yl)oxy)benzamide

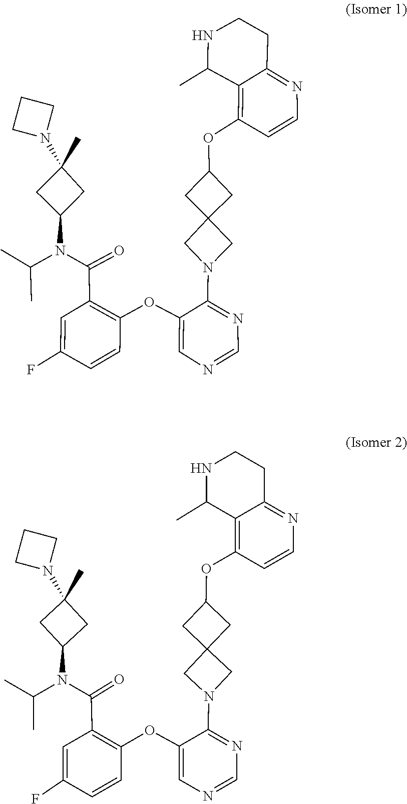 [SMILES: CC1NCCc2nccc(OC3CC4(C3)CN(c3ncncc3Oc3ccc(F)cc3C(=O)N(C(C)C)[C@H]3C[C@](C)(N5CCC5)C3)C4)c21 ; CCC1NCCc2nccc(OC3CC4(C3)CN(c3ncncc3Oc3ccc(F)cc3C(=O)N(C(C)C)[C@H]3C[C@](C)(N5CCC5)C3)C4)c21]

The diastereomeric mixture of N-((1r,3r)-3-(azetidin-1-yl)-3-methylcyclobutyl)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide (300 mg, 0.457 mmol) was purified by chiral Prep SFC (Method R). The pure fractions were concentrated under reduced pressure and lyophilized to obtain (D1)-N-((1r,3r)-3-(azetidin-1-yl)-3-methylcyclobutyl)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide (70 mg, 23.11% yield) as a solid and impure (D2)-N-((1r,3r)-3-(Azetidin-1-yl)-3-methylcyclobutyl)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide (77 mg). This impure isomer was repurified by reverse phase chromatography using a SiliCycle SiliaSep C18 column (40 g) on Biotage isolera one (Mobile phase A: 10 mM NH₄HCO₃ in water, B: ACN) to obtain (D2)-N-((1r,3r)-3-(azetidin-1-yl)-3-methylcyclobutyl)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide (34 mg, 11.29% yield) as a solid.

Isomer 1. ¹H NMR (400 MHz, DMSO-d₆): δ 8.32-8.22 (m, 1H), 8.15 (d, J=5.5 Hz, 1H), 7.79-7.59 (m, 1H), 7.33-7.05 (m, 3H), 6.68-6.56 (m, 1H), 4.73 (quin, J=6.0 Hz, 1H), 4.38-4.11 (m, 4H), 4.05 (q, J=6.3 Hz, 1H), 4.01-3.63 (m, 2H), 3.16-3.03 (m, 4H), 2.97-2.73 (m, 6H), 2.66-2.57 (m, 2H), 2.29-1.97 (m, 4H), 1.92-1.84 (m, 1H), 1.82-1.70 (m, 2H), 1.48-0.86 (m, 12H); LCMS (Method H): Rt=1.13 min, m/z=656.3 [M+H]⁺; HPLC (Method C): Rt=7.93 min, 99.05%; Chiral SFC (Method AZ): Rt=4.67 min, 100%.

Isomer 2. ¹H NMR (400 MHz, DMSO-d₆): δ 8.30-8.24 (m, 1H), 8.16 (d, J=5.5 Hz, 1H), 7.78-7.61 (m, 1H), 7.33-7.05 (m, 3H), 6.69-6.58 (m, 1H), 4.74 (quin, J=6.3 Hz, 1H), 4.35-4.12 (m, 4H), 4.08 (q, J=6.0 Hz, 1H), 4.02-3.63 (m, 2H), 3.19-3.05 (m, 4H), 2.97-2.74 (m, 6H), 2.69-2.63 (m, 2H), 2.30-1.98 (m, 4H), 1.94-1.84 (m, 1H), 1.83-1.71 (m, 2H), 1.48-0.90 (m, 12H); LCMS (Method H): Rt=1.40 min, m/z=656.4 [M+H]⁺; HPLC (Method C): Rt=7.89 min, 99.61%; Chiral SFC (Method AZ): Peak 2: Rt=5.57 min, 98.89%.

### Example 158. (D1)-5-Fluoro-N-isopropyl-N-((1r,3r)-3-methyl-3-(pyrrolidin-1-yl)cyclobutyl)-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide (Compound No. 158)

### Example 159. (D2)-5-Fluoro-N-isopropyl-N-((1r,3r)-3-methyl-3-(pyrrolidin-1-yl)cyclobutyl)-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide (Compound No. 159)

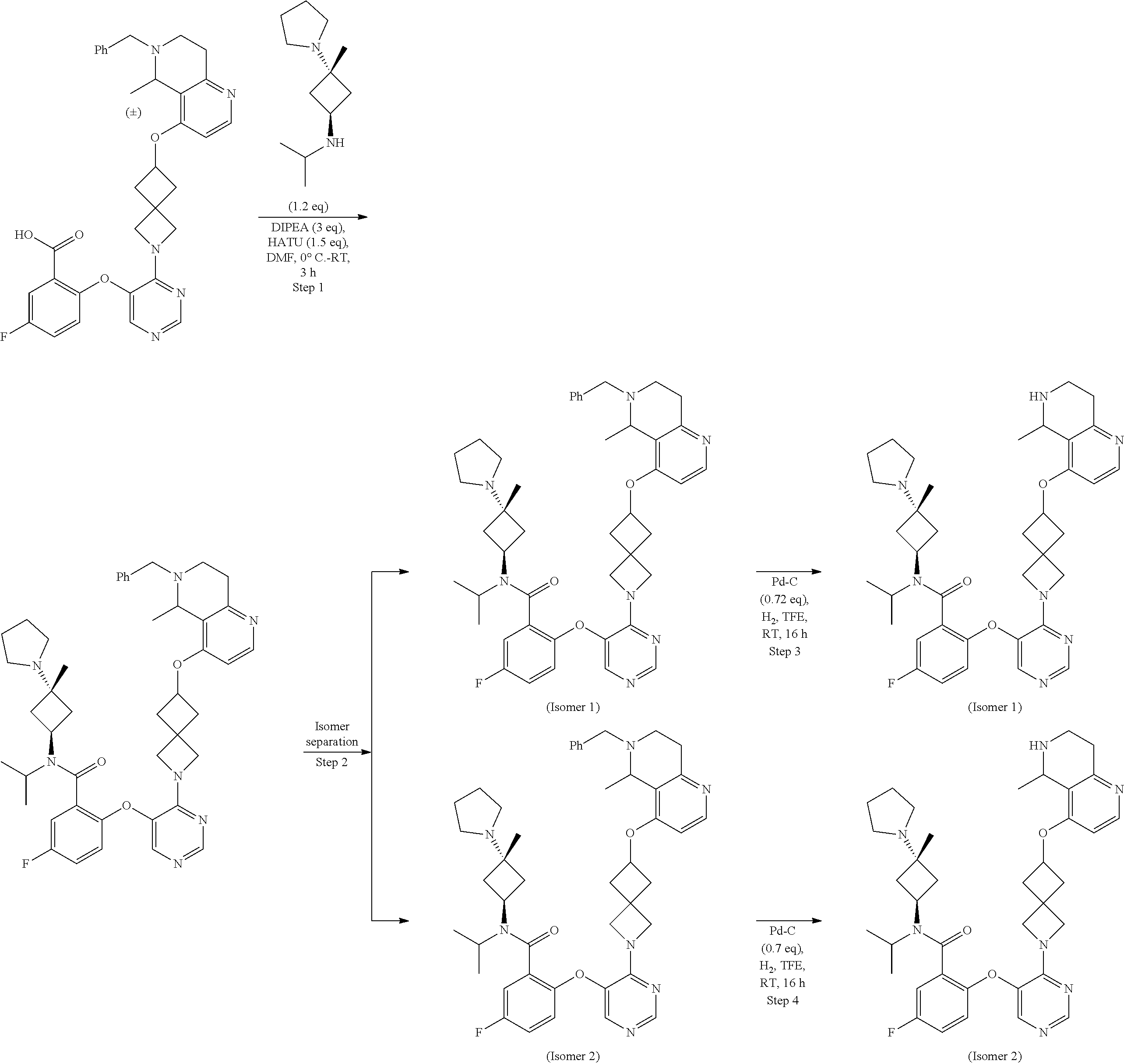

### Step 1. 2-((4-(6-((6-Benzyl-5-methyl-5,6,7,8-tetrahydro-1,6-naphthyridin-4-yl)oxy)-2-azaspiro[3.3]hepta-2-yl)pyrimidin-5-yl)oxy)-5-fluoro-N-isopropyl-N-((1r,3r)-3-methyl-3-(pyrrolidin-1-yl)cyclobutyl)benzamide

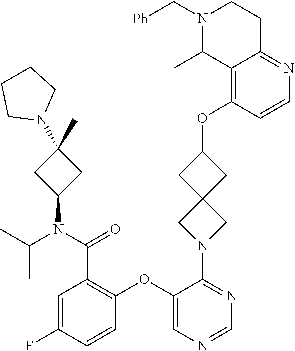 [SMILES: CC1c2c(OC3CC4(C3)CN(c3ncncc3Oc3ccc(F)cc3C(=O)N(C(C)C)[C@H]3C[C@](C)(N5CCCC5)C3)C4)ccnc2CCN1Cc1ccccc1]

The reaction was performed following the same procedure in two batches.

This compound was synthesized following the procedure described for the synthesis of Example 1, step 2, starting with 1 equivalent of (±)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.2 equivalents of (1r,3r)—N-isopropyl-3-methyl-3-(pyrrolidin-1-yl)cyclobutan-1-amine. The reaction mixtures from both batches were combined and concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain 2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide as a sticky solid.

Yield: 93%; LCMS (Method A): Rt=2.20 min, m/z=760.3 [M+H]⁺; HPLC (Method C): Rt=11.01 min, 93.87%; Chiral SFC (Method AY): Peak 1: Rt=7.20 min, 46.39%; Peak 2: Rt=8.84 min, 48.69%.

### Step 2. (D1)-2-((4-(6-((6-Benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide and (D2)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide

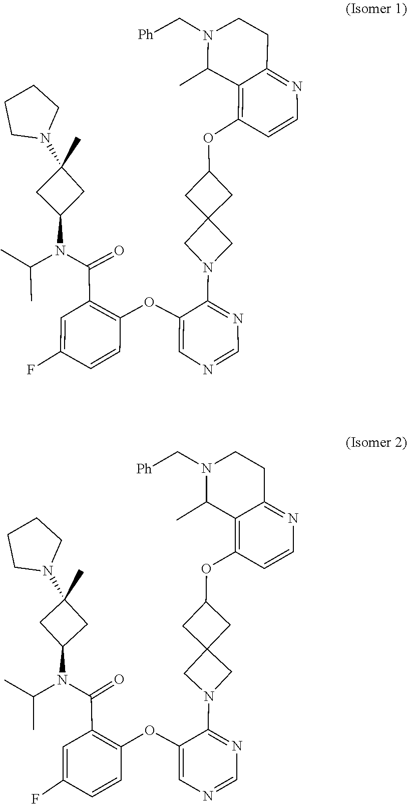 [SMILES: CC1c2c(OC3CC4(C3)CN(c3ncncc3Oc3ccc(F)cc3C(=O)N(C(C)C)[C@H]3C[C@](C)(N5CCCC5)C3)C4)ccnc2CCN1Cc1ccccc1 ; CCC1c2c(OC3CC4(C3)CN(c3ncncc3Oc3ccc(F)cc3C(=O)N(C(C)C)[C@H]3C[C@](C)(N5CCCC5)C3)C4)ccnc2CCN1Cc1ccccc1]

The diastereomeric mixture of 2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide (1.2 g, 1.579 mmol) was purified by chiral Prep SFC (Method Q) to afford (D1)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide (300 mg, 24.94% yield) and (D2)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide (360 mg, 28.4% yield) as solids.

Isomer 1. ¹H NMR (400 MHz, DMSO-d₆): δ 8.30-8.23 (m, 1H), 8.21-8.16 (m, 1H), 7.78-7.55 (m, 1H), 7.37-7.21 (m, 6H), 7.18-7.05 (m, 2H), 6.75-6.61 (m, 1H), 4.81-4.69 (m, 1H), 4.36-3.94 (m, 5H), 3.87-3.56 (m, 4H), 3.12-2.98 (m, 11H), 2.92-2.70 (m, 5H), 2.66-2.58 (m, 2H), 2.37 (br s, 2H), 2.27-2.13 (m, 3H), 2.01-1.52 (m, 6H), 1.46-0.89 (m, 12H), two protons merged with solvent peaks; LCMS (Method A): Rt=2.27 min, m/z=760.3 [M+H]⁺; HPLC (Method C): Rt=11.15 min, 99.74%; Chiral SFC (Method AY): Rt=7.31 min, 99.57%.

Isomer 2. ¹H NMR (400 MHz, DMSO-d₆): δ 8.30-8.24 (m, 1H), 8.24-8.14 (m, 1H), 7.78-7.58 (m, 1H), 7.36-7.22 (m, 6H), 7.19-7.06 (m, 2H), 6.71-6.61 (m, 1H), 4.74 (quin, J=6.5 Hz, 1H), 4.35-3.94 (m, 5H), 3.90-3.58 (m, 4H), 3.10-3.00 (m, 1H), 2.92-2.70 (m, 5H), 2.64-2.55 (m, 2H), 2.37 (br s, 2H), 2.27-2.14 (m, 3H), 2.00-1.55 (m, 6H), 1.48-0.89 (m, 12H), two protons merged with solvent peaks; LCMS (Method H): Rt=1.38 min, m/z=760.4 [M+H]⁺; HPLC (Method G): Rt=6.90 min, 94.64%; Chiral SFC (Method AY): Rt=8.89 min, 97.52%.

### Step 3. (D1)-5-Fluoro-N-isopropyl-N-((1r,3r)-3-methyl-3-(pyrrolidin-1-yl)cyclobutyl)-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide

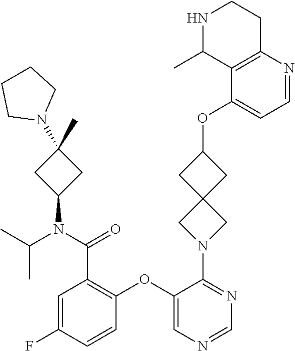 [SMILES: CC1NCCc2nccc(OC3CC4(C3)CN(c3ncncc3Oc3ccc(F)cc3C(=O)N(C(C)C)[C@H]3C[C@](C)(N5CCCC5)C3)C4)c21]

This compound was synthesized following the procedure described for the synthesis of Example 5, step 1, starting with 1 equivalent of (D1)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide, except that 0.72 equivalent of Pd—C was used. After filtration, the filtrate was concentrated under reduced pressure. The residue was diluted with sodium bicarbonate and the mixture was extracted with 10% MeOH in DCM. The combined organic layer was dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford (D1)-5-fluoro-N-isopropyl-N-((1r,3r)-3-methyl-3-(pyrrolidin-1-yl)cyclobutyl)-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 63.5%; ¹H NMR (400 MHz, DMSO-d₆): δ 8.31-8.23 (m, 1H), 8.15 (d, J=5.5 Hz, 1H), 7.78-7.58 (m, 1H), 7.32-7.04 (m, 3H), 6.69-6.58 (m, 1H), 4.73 (quin, J=6.5 Hz, 1H), 4.36-4.12 (m, 4H), 4.05 (br q, 1=6.6 Hz, 11H) 3.89-3.63 (m, 2H), 3.12-2.99 (m, 1H), 2.93-2.74 (m, 4H), 2.71-2.62 (m, 2H), 2.37 (br s, 2H), 2.30-1.91 (m, 5H), 1.85-1.74 (m, 1H), 1.72-1.53 (m, 4H), 1.50-0.90 (m, 12H), two protons merged with solvent peaks; LCMS (Method H): Rt=1.26 min, m/z=670.4 [M+H]⁺; HPLC (Method G): Rt=6.37 min, 99.40%; Chiral SFC (Method J): Rt=4.86 min, 98.01%.

### Step 4. (D2)-5-Fluoro-N-isopropyl-N-((1r,3r)-3-methyl-3-(pyrrolidin-1-yl)cyclobutyl)-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide

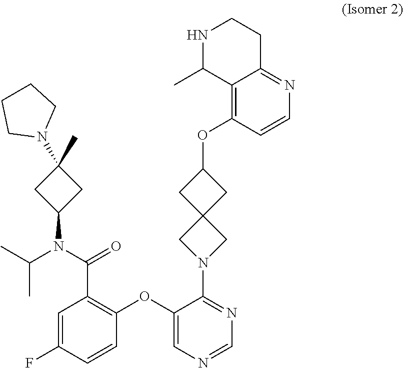 [SMILES: CC1NCCc2nccc(OC3CC4(C3)CN(c3ncncc3Oc3ccc(F)cc3C(=O)N(C(C)C)[C@H]3C[C@](C)(N5CCCC5)C3)C4)c21]

This compound was synthesized following the procedure described for the synthesis of Example 5, step 1, starting with 1 equivalent of (D2)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide, except that 0.7 equivalent of Pd—C was used. After filtration, the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford (D2)-5-fluoro-N-isopropyl-N-((1r,3r)-3-methyl-3-(pyrrolidin-1-yl)cyclobutyl)-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 52.9%; ¹H NMR (400 MHz, DMSO-d₆): δ 8.34-8.22 (m, 1H), 8.15 (br d, J=5.4 Hz, 1H), 7.78-7.56 (m, 1H), 7.31-7.05 (m, 3H), 6.69-6.52 (m, 1H), 4.74 (quin, J=6.4 Hz, 1H), 4.35-4.11 (m, 4H), 4.06 (q, J=6.3 Hz, 1H), 3.89-3.63 (m, 2H), 3.13-3.02 (m, 1H), 2.94-2.74 (m, 4H), 2.71-2.64 (m, 2H), 2.37 (br s, 2H), 2.29-1.90 (m, 5H), 1.85-1.74 (m, 1H), 1.73-1.54 (m, 4H), 1.48-0.90 (m, 12H), two protons merged with solvent peaks; LCMS (Method H): Rt=1.25 min, m/z=670.4 [M+H]⁺; HPLC (Method G): Rt=6.36 min, 98.83%; Chiral SFC (Method J): Rt=5.12 min, 97.74%.

### Example 160. (D1)-5-Fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-methyl-5-azaspiro[3.4]octan-2-yl)benzamide (Compound No. 160)

### Example 161. (D2)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-methyl-5-azaspiro[3.4]octan-2-yl)benzamide (Compound No. 161)

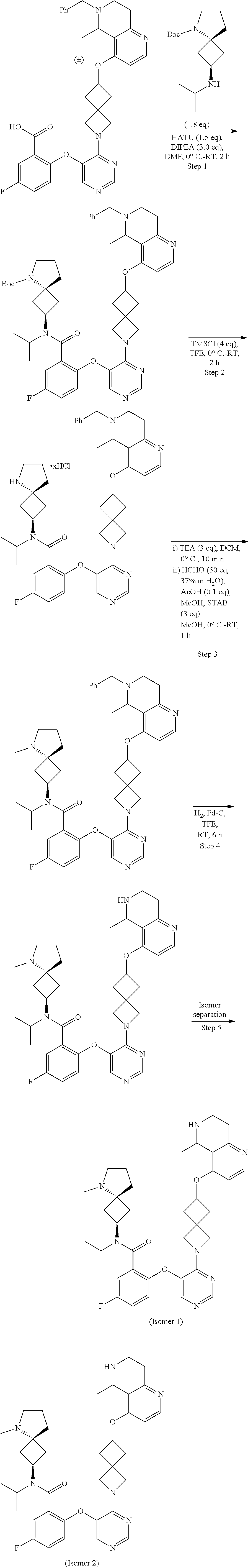

### Step 1. tert-Butyl(2s,4r)-2-(2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.4]octane-5-carboxylate

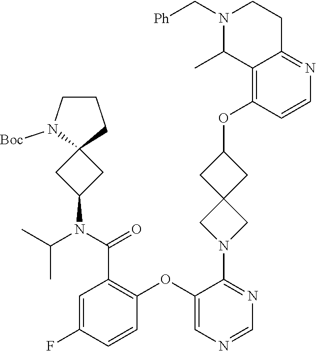 [SMILES: CC1c2c(OC3CC4(C3)CN(c3ncncc3Oc3ccc(F)cc3C(=O)N(C(C)C)[C@H]3C[C@@]5(CCCN5C(=O)OC(C)(C)C)C3)C4)ccnc2CCN1Cc1ccccc1]

This compound was synthesized following the procedure described for the synthesis of Example 1, step 2, starting with 1 equivalent of (±)-2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.8 equivalents of tert-butyl(2s,4r)-2-(isopropylamino)-5-azaspiro[3.4]octane-5-carboxylate. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl(2s,4r)-2-(2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.4]octane-5-carboxylate as a solid.

Yield: 64.3%; ¹H NMR (400 MHz, DMSO-d₆): δ 8.27 (s, 1H), 8.20 (d, J=5.6 Hz, 1H), 7.79-7.76 (m, 1H), 7.37-7.20 (m, 7H), 7.17-7.05 (m, 1H), 6.66 (d, J=5.5 Hz, 1H), 4.77-4.69 (m, 1H), 4.39-4.04 (m, 5H), 4.02-3.94 (m, 1H), 3.80-3.57 (m, 2H), 3.28-3.19 (m, 2H), 3.10-3.00 (m, 2H), 2.92-2.69 (m, 6H), 2.65-2.56 (m, 2H), 2.32-2.12 (m, 4H), 1.61-1.41 (m, 11H), 1.27-0.88 (m, 9H), one proton merged with solvent peaks; LCMS (Method A): Rt=2.57 min, m/z=832.4 [M+H]⁺, 98.15%.

### Step 2. 2-((4-(6-((6-Benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-azaspiro[3.4]octan-2-yl)benzamide xHCl

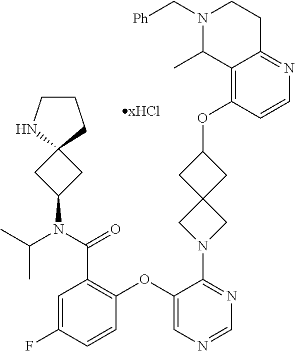 [SMILES: CC1c2c(OC3CC4(C3)CN(c3ncncc3Oc3ccc(F)cc3C(=O)N(C(C)C)[C@H]3C[C@@]5(CCCN5)C3)C4)ccnc2CCN1Cc1ccccc1]

This compound was synthesized following the procedure described for the synthesis of Example 20, Step 3, starting with 1 equivalent of tert-butyl(2s,4r)-2-(2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.4]octane-5-carboxylate. After completion, the reaction was concentrated under reduced pressure and the residue was triturated with EtOAc. The organic layer was decanted and the solid was dried under vacuum to obtain crude 2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-azaspiro[3.4]octan-2-yl)benzamide xHCl as a solid. This material was used without further purification.

Yield: 96%; LCMS (Method A): Rt=2.12 min, m/z=732.3 [M+H]⁺, 97.29%.

### Step 3. 2-((4-(6-((6-Benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide

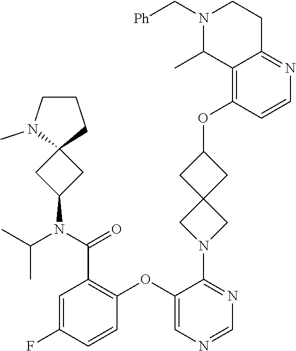 [SMILES: CC1c2c(OC3CC4(C3)CN(c3ncncc3Oc3ccc(F)cc3C(=O)N(C(C)C)[C@H]3C[C@@]5(CCCN5C)C3)C4)ccnc2CCN1Cc1ccccc1]

This compound was synthesized following the procedure described for the synthesis of Example 20, step 4, starting with 1 equivalent of 2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-azaspiro[3.4]octan-2-yl)benzamide xHCl, except that 3 equivalents of TEA, 0.1 equivalent of AcOH, 50 equivalents of formaldehyde, and 3 equivalents of STAB were used. After work-up, the crude product was purified by reverse phase chromatography using a SiliCycle SiliaSep C18 column (80 g) on Biotage isolera one (Mobile phase A: 10 mM NH₄HCO₃ in water, B: ACN) to obtain 2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide as a solid.

Yield: 83%; ¹H NMR (400 MHz, DMSO-d₆): δ 8.29-8.24 (m, 1H), 8.23-8.16 (m, 1H), 7.78-7.57 (m, 1H), 7.37-7.21 (m, 7H), 7.21-7.06 (m, 1H), 6.70-6.63 (m, 1H), 4.81-4.69 (m, 1H), 4.36-4.09 (m, 4H), 4.04-3.83 (m, 1H), 3.81-3.56 (m, 4H), 3.08-3.01 (m, 1H), 2.99-2.69 (m, 4H), 2.62-2.57 (m, 1H), 2.47-2.39 (m, 2H), 2.33 (br s, 3H), 2.30-2.12 (m, 3H), 2.09-1.85 (m, 3H), 1.75-1.68 (m, 1H), 1.60-1.41 (m, 3H), 1.34-0.89 (m, 9H); LCMS (Method A): Rt=2.20 min, m/z=746.3 [M+H]⁺, 99.10%.

### Step 4. 5-Fluoro-N-isopropyl-2-((4-(6-((S-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-methyl-5-azaspiro[3.4]octan-2-yl)benzamide

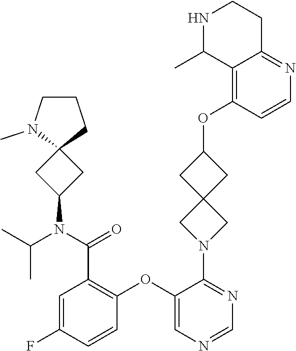 [SMILES: CC1NCCc2nccc(OC3CC4(C3)CN(c3ncncc3Oc3ccc(F)cc3C(=O)N(C(C)C)[C@H]3C[C@@]5(CCCN5C)C3)C4)c21]

This compound was synthesized following the procedure described for the synthesis of Example 20, step 5, starting with 1 equivalent of 2-((4-(6-((6-benzyl-5-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide, except that 0.7 equivalent of Pd—C was used. After completion, the reaction was filtered through a Celite® pad, and the pad was washed with TFE. The filtrate was concentrated under reduced pressure to obtain crude 5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-methyl-5-azaspiro[3.4]octan-2-yl)benzamide as a solid.

Yield: 95%; LCMS (Method H): Rt=1.07 min, m/z=656.4 [M+H]⁺, 98.74%.

### Step 5. (D1)-5-Fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-methyl-5-azaspiro[3.4]octan-2-yl)benzamide and (D2)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-methyl-5-azaspiro[3.4]octan-2-yl)benzamide

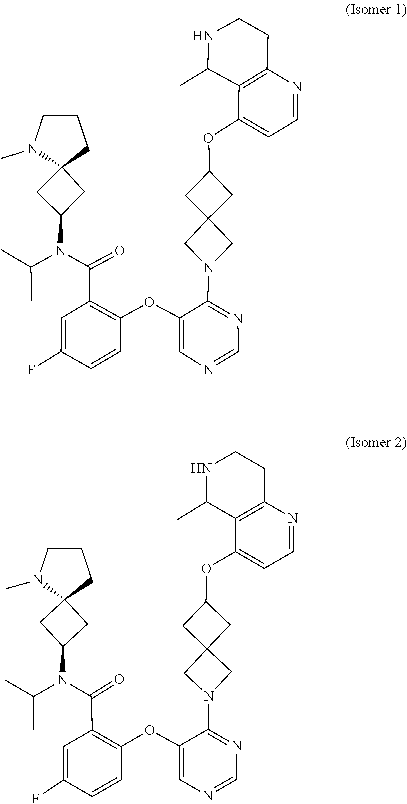 [SMILES: CC1NCCc2nccc(OC3CC4(C3)CN(c3ncncc3Oc3ccc(F)cc3C(=O)N(C(C)C)[C@H]3C[C@@]5(CCCN5C)C3)C4)c21 ; CCC1NCCc2nccc(OC3CC4(C3)CN(c3ncncc3Oc3ccc(F)cc3C(=O)N(C(C)C)[C@H]3C[C@@]5(CCCN5C)C3)C4)c21]

The diastereomeric mixture of 5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-methyl-5-azaspiro[3.4]octan-2-yl)benzamide (420 mg, 0.640 mmol) was purified by chiral Prep SFC (Method X) to obtain (D1)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-methyl-5-azaspiro[3.4]octan-2-yl)benzamide (125 mg, 33.9% yield) and (D2)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-methyl-5-azaspiro[3.4]octan-2-yl)benzamide (81 mg, 1.94% yield) as solids.

Isomer 1. ¹H NMR (400 MHz, DMSO-d₆): δ 8.29-8.23 (m, 1H), 8.21-8.15 (m, 1H), 7.80-7.56 (m, 1H), 7.33-7.22 (m, 2H), 7.20-7.06 (m, 1H), 6.68-6.60 (m, 1H), 4.76-4.70 (m, 1H), 4.38-4.01 (m, 5H), 3.96-3.61 (m, 2H), 3.12-3.02 (m, 1H), 2.99-2.70 (m, 4H), 2.69-2.64 (m, 2H), 2.45-2.39 (m, 2H), 2.33 (s, 3H), 2.26-2.16 (m, 3H), 2.08-1.86 (m, 4H), 1.74-1.70 (m, 1H), 1.58-0.90 (m, 11H); LCMS (Method H): Rt=1.09 min, m/z=656.3 [M+H]⁺; HPLC (Method G): Rt=6.27 min, 99.71%; Chiral SFC (Method AAA)=3.76 min; 100%.

Isomer 2. H NMR (400 MHz, DMSO-d₆): δ 8.31-8.23 (m, 1H), 8.21-8.13 (m, 1H), 7.80-7.55 (m, 1H), 7.33-7.21 (m, 2H), 7.20-7.04 (m, 1H), 6.71-6.61 (m, 1H), 4.74-4.68 (m, 1H), 4.39-4.07 (m, 5H), 3.95-3.60 (m, 2H), 3.19-3.08 (m, 1H), 3.04-2.90 (m, 2H), 2.87-2.69 (m, 5H), 2.46-2.39 (m, 2H), 2.35 (s, 3H), 2.29-2.15 (m, 3H), 2.11-1.87 (m, 4H), 1.78-1.67 (m, 1H), 1.59-0.90 (m, 11H); LCMS (Method H): Rt=1.05 min, m/z=656.3 [M+H]⁺; HPLC (Method B): Rt=6.12 min, 99.17%; Chiral SFC (Method AAA)=4.98 min; 98.43%.

### Example 162. (D1)-N-((1r,3r)-3-(Dimethylamino)-3-methylcyclobutyl)-5-fluoro-2-((5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-isopropylbenzamide (Compound No. 162)

### Example 163. (D2)-N-((1r,3r)-3-(Dimethylamino)-3-methylcyclobutyl)-5-fluoro-2-((5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-isopropylbenzamide (Compound No. 163)

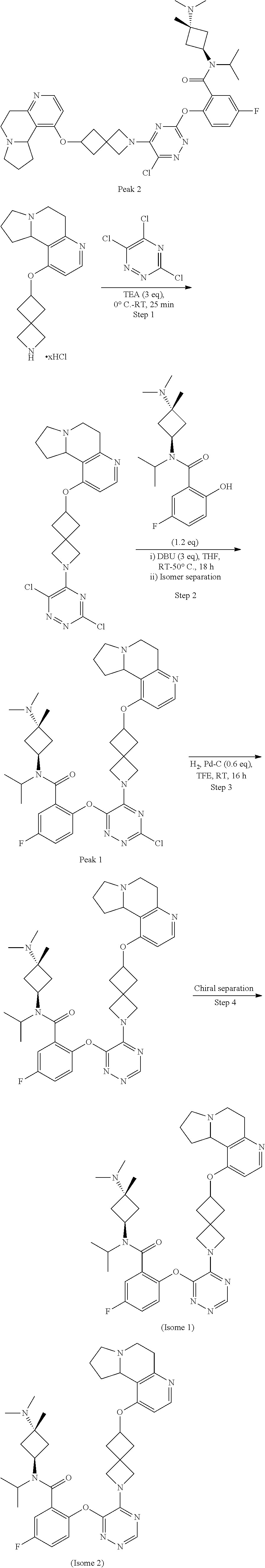

### Step 1. (±)-1-((2-(3,6-Dichloro-1,2,4-triazin-S-yl)-2-azaspiro[3.3]heptan-6-yl)oxy)-5,6,8,9,10,10α-hexahydropyrrolo[2,1-f][1,6]naphthyridine

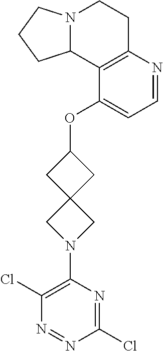 [SMILES: Clc1nnc(Cl)c(N2CC3(CC(Oc4ccnc5c4C4CCCN4CC5)C3)C2)n1]

To a stirred solution of (±)-1-((2-azaspiro[3.3]heptan-6-yl)oxy)-5,6,8,9,10,10a-hexahydropyrrolo[2,1-f][1,6]naphthyridine xHCl (950 mg, 2.95 mmol) in DCM (30 mL) under a nitrogen atmosphere, TEA (1.234 mL, 8.86 mmol) was added at 0° C. The reaction was stirred for 5 min at the same temperature. To this reaction mixture, 3,5,6-trichloro-1,2,4-triazine (544 mg, 2.95 mmol) was added at 0° C. and the reaction was stirred at RT for 20 min. The reaction progress was monitored by LCMS and TLC (10% MeOH in DCM). After completion, the reaction was diluted with water (10 mL), and the mixture was extracted with 10% MeOH in DCM (2×10 mL). The combined organic layer was dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford (±)-1-((2-(3,6-dichloro-1,2,4-triazin-5-yl)-2-azaspiro[3.3]heptan-6-yl)oxy)-5,6,8,9,10,10a-hexahydropyrrolo[2,1-f][1,6]naphthyridine (260 mg, 15.63% yield) as a solid. ¹H NMR (400 MHz, DMSO-d₆): δ 8.19 (d, J=5.6 Hz, 1H), 6.69 (d, J=5.8 Hz, 1H), 4.85-4.72 (m, 3H), 4.28-4.15 (m, 2H), 3.68 (br t, J=8.0 Hz, 1H), 3.01-2.75 (m, 6H), 2.73-2.64 (m, 2H), 2.46-2.40 (m, 1H), 2.35-2.25 (m, 2H), 1.90-1.70 (m, 2H), 1.57-1.41 (m, 1H); LCMS (Method A): Rt=1.65 min, m/z=433.0 [M+H]⁺, 76.88%.

### Step 2. 2-((3-Chloro-5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-N-((trans)-3-(dimethylamino)-3-methylcyclobutyl)-5-fluoro-N-isopropylbenzamide and 2-((6-chloro-5-(6-((5,6,8,9,10,10α-hexahydropyrrolo[2,1-f][1,6]naphthyridin-1-yl)oxy)-2-azaspiro[3.3]heptan-2-yl)-1,2,4-triazin-3-yl)oxy)-N-((trans)-3-(dimethylamino)-3-methylcyclobutyl)-5-fluoro-N-isopropylbenzamide

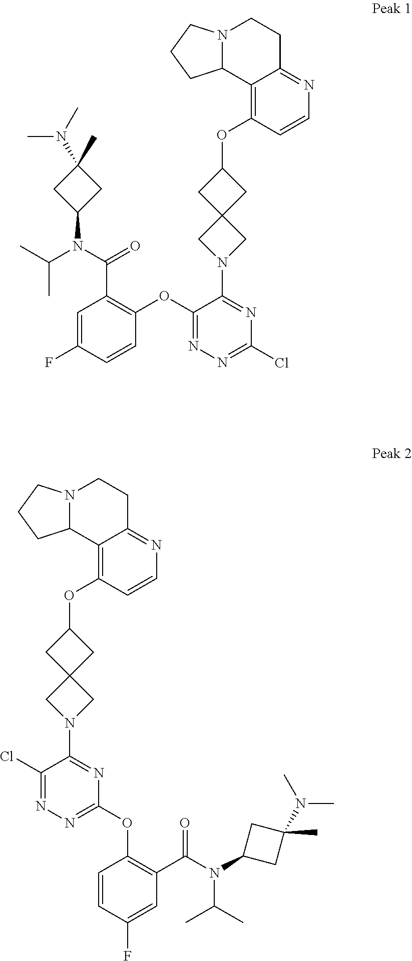 [SMILES: CC(C)N(C(=O)c1cc(F)ccc1Oc1nnc(Cl)nc1N1CC2(CC(Oc3ccnc4c3C3CCCN3CC4)C2)C1)[C@H]1C[C@](C)(N(C)C)C1 ; CC(C)N(C(=O)c1cc(F)ccc1Oc1nnc(Cl)c(N2CC3(CC(Oc4ccnc5c4C4CCCN4CC5)C3)C2)n1)[C@H]1C[C@](C)(N(C)C)C1]

This compound was synthesized following the procedure described for the synthesis of Example 59, step 4, starting with 1 equivalent of (±)-1-((2-(3,6-dichloro-1,2,4-triazin-5-yl)-2-azaspiro[3.3]heptan-6-yl)oxy)-5,6,8,9,10,10a-hexahydropyrrolo[2,1-f][1,6]naphthyridine and 1.2 equivalents of N-((1r,3r)-3-(dimethylamino)-3-methylcyclobutyl)-5-fluoro-2-hydroxy-N-isopropylbenzamide. The reaction was stirred at 50° C. After work-up, the crude compound was purified by reverse phase chromatography using a SiliCycle SiliaSep C18 column (120 g) on Biotage isolera one (Mobile phase A: 10 mM NH₄HCO₃ in water, B: ACN). The pure fractions were concentrated under reduced pressure to afford a mixture of 2-((6-chloro-5-(6-((5,6,8,9,10,10α-hexahydropyrrolo[2,1-f][1,6]naphthyridin-1-yl)oxy)-2-azaspiro[3.3]heptan-2-yl)-1,2,4-triazin-3-yl)oxy)-N-((1r,3r)-3-(dimethylamino)-3-methylcyclobutyl)-5-fluoro-N-isopropylbenzamide and the other regioisomer (340 mg). The mixture of regioisomers (340 mg) were purified by achiral Prep SFC (Method C) to obtain both regioisomers.

Peak 1. 2-((3-Chloro-5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-((1r,3r)-3-(dimethylamino)-3-methylcyclobutyl)-5-fluoro-N-isopropylbenzamide (10.74% yield) as a solid. LCMS (Method A): Rt=1.93 min, m/z=705.1 [M+H]⁺, 99.81%; Achiral SFC (Method A): Rt=3.63 min, 98.99%.

Peak 2. 2-((6-Chloro-5-(6-((5,6,8,9,10,10a-hexahydropyrrolo[2,1-f][1,6]naphthyridin-1-yl)oxy)-2-azaspiro[3.3]heptan-2-yl)-1,2,4-triazin-3-yl)oxy)-N-((1r,3r)-3-(dimethylamino)-3-methylcyclobutyl)-5-fluoro-N-isopropylbenzamide (6.09% yield) as a solid. LCMS (Method A): Rt=2.04 min, m/z=705.1 [M+H]⁺; 99.15% Achiral SFC (Method A): Rt=4.58 min, 90.31%.

### Step 3. N-((1r,3r)-3-(Dimethylamino)-3-methylcyclobutyl)-5-fluoro-2-((5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-N-isopropylbenzamide

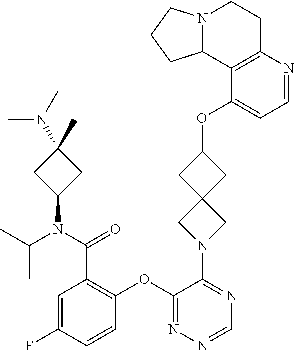 [SMILES: CC(C)N(C(=O)c1cc(F)ccc1Oc1nncnc1N1CC2(CC(Oc3ccnc4c3C3CCCN3CC4)C2)C1)[C@H]1C[C@](C)(N(C)C)C1]

This compound was synthesized following the procedure described for the synthesis of Example 59, step 5, starting with 1 equivalent of 2-((3-chloro-5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-((1r,3r)-3-(dimethylamino)-3-methylcyclobutyl)-5-fluoro-N-isopropylbenzamide, except that 0.6 equivalent of Pd—C was used. After completion, the reaction was filtered through a Celite® pad, and the pad was washed with MeOH. The filtrate was concentrated under reduced pressure. The crude product was purified by reverse phase chromatography using a SiliCycle SiliaSep C18 column (40 g) on Biotage isolera one (Mobile phase A: 10 mM NH₄HCO₃ in water, B: ACN) to obtain N-((1r,3r)-3-(dimethylamino)-3-methylcyclobutyl)-5-fluoro-2-((5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-N-isopropylbenzamide as a solid.

Yield: 56.4%; ¹H NMR (400 MHz, DMSO-d₆): δ 8.46 (s, 1H), 8.21-8.14 (m, 1H), 7.49-7.12 (m, 3H), 6.75-6.59 (m, 1H), 4.85-4.75 (m, 1H), 4.73-4.48 (m, 2H), 4.30-3.84 (m, 3H), 3.81-3.49 (m, 3H), 3.02-2.60 (m, 9H), 2.44-2.23 (m, 2H), 2.16-1.92 (m, 8H), 1.87-1.62 (m, 3H), 1.58-1.35 (m, 3H), 1.25-0.53 (m, 7H); LCMS (Method A): Rt=1.98 min, m/z=671.2 [M+H]⁺, 96.23%; Chiral SFC (Method AT)=Peak 1: Rt=4.17 min, 50.91%; Peak 2: Rt=8.71 min, 49.10%.

### Step 4. (D1)-N-((1r,3r)-3-(Dimethylamino)-3-methylcyclobutyl)-5-fluoro-2-((5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-N-isopropylbenzamide and (D2)-N-((1r,3r)-3-(dimethylamino)-3-methylcyclobutyl)-5-fluoro-2-((5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-isopropylbenzamide

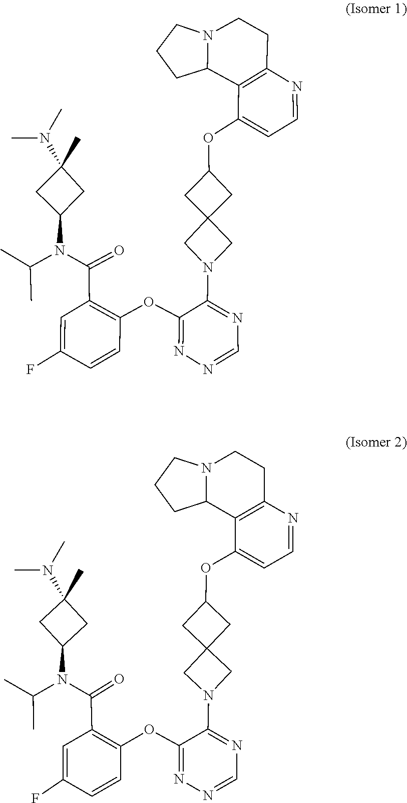 [SMILES: CC(C)N(C(=O)c1cc(F)ccc1Oc1nncnc1N1CC2(CC(Oc3ccnc4c3C3CCCN3CC4)C2)C1)[C@H]1C[C@](C)(N(C)C)C1 ; CC(C)N(C(=O)c1cc(F)ccc1Oc1nncnc1N1CC2(CC(Oc3ccnc4c3C3CCCN3CC4)C2)C1)[C@H]1C[C@](C)(N(C)C)C1]

The diastereomeric mixture of N-((1r,3r)-3-(dimethylamino)-3-methylcyclobutyl)-5-fluoro-2-((5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-N-isopropylbenzamide (78 mg, 0.116 mmol) was purified by chiral Prep SFC (Method K) to obtain (D1)-N-((1r,3r)-3-(dimethylamino)-3-methylcyclobutyl)-5-fluoro-2-((5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-N-isopropylbenzamide (26 mg, 33.3% yield) and (D2)-N-((1r,3r)-3-(dimethylamino)-3-methylcyclobutyl)-5-fluoro-2-((5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-isopropylbenzamide (23 mg, 29.5% yield) as solids.

Isomer 1. ¹H NMR (400 MHz, DMSO-d₆): δ 8.46 (s, 1H), 8.18 (br d, J=5.3 Hz, 1H), 7.49-7.10 (m, 3H), 6.73-6.61 (m, 1H), 4.85-4.75 (m, 1H), 4.73-4.50 (m, 2H), 4.33-3.87 (m, 3H), 3.81-3.49 (m, 3H), 3.05-2.63 (m, 9H), 2.44-2.23 (m, 2H), 2.17-1.91 (m, 8H), 1.87-1.67 (m, 3H), 1.60-1.35 (m, 3H), 1.29-0.56 (m, 7H); LCMS (Method C): Rt=1.43 min, m/z=671.5 [M+H]⁺; HPLC (Method E): Rt=8.89 min, 97.44%; Chiral SFC (Method AT): Rt=3.84 min, 100%.

Isomer 2. ¹H NMR (400 MHz, DMSO-d₆): δ 8.46 (s, 1H), 8.18 (br d, J=5.1 Hz, 1H), 7.49-7.08 (m, 3H), 6.76-6.60 (m, 1H), 4.87-4.76 (m, 1H), 4.72-4.49 (m, 2H), 4.30-3.88 (m, 3H), 3.79-3.53 (m, 3H), 3.03-2.62 (m, 9H), 2.44-2.24 (m, 2H), 2.16-1.92 (m, 8H), 1.85-1.65 (m, 3H), 1.58-1.35 (m, 3H), 1.26-0.55 (m, 7H); LCMS (Method C): Rt=1.45 min, m/z=671.5 [M+H]⁺; HPLC (Method E): Rt=8.89 min, 98.60%; Chiral SFC (Method AT): Rt=7.86 min, 99.17%.

### Example 164. N-((1r,3r)-3-(Dimethylamino)-3-methylcyclobutyl)-5-fluoro-N-isopropyl-2-((5-(6-((1,2,3,4-tetrahydroisoquinolin-8-yl)oxy)-2-azaspiro[3.3]heptan-2-yl)-1,2,4-triazin-6-yl)oxy)benzamide (Compound No. 164)

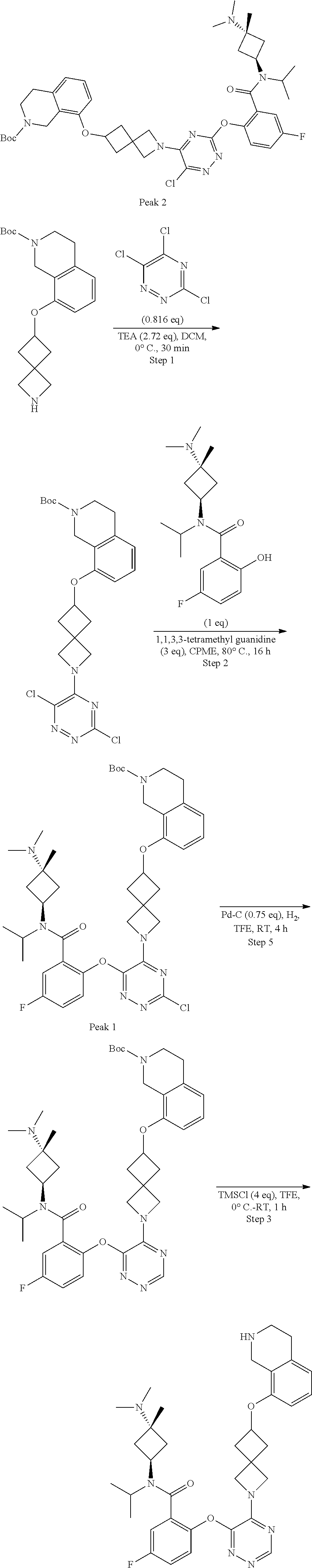

### Step 1. tert-Butyl 8-((2-(3,6-dichloro-1,2,4-triazin-5-yl)-2-azaspiro[3.3]heptan-6-yl)oxy)-3,4-dihydroisoquinoline-2(1H)-carboxylate

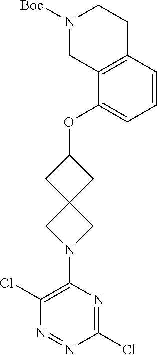 [SMILES: CC(C)(C)OC(=O)N1CCc2cccc(OC3CC4(C3)CN(c3nc(Cl)nnc3Cl)C4)c2C1]

To a stirred solution of tert-butyl 8-((2-azaspiro[3.3]heptan-6-yl)oxy)-3,4-dihydroisoquinoline-2(1H)-carboxylate (5.7 g, 14.96 mmol) in dry DCM (2 mL), TEA (6.26 mL, 44.9 mmol) was added at 0° C. To this reaction mixture, 3,5,6-trichloro-1,2,4-triazine (2.483 g, 13.47 mmol) was added in one portion at 0° C. The reaction was stirred at 0° C. for 30 min. The reaction progress was monitored by TLC (10% MeOH in DCM). The reaction was diluted with sodium bicarbonate solution (10 mL) and the mixture was extracted with DCM (3×50 mL). The combined organic layer was washed with brine (50 mL), dried over anhydrous sodium sulfate and filtered, and the filtrate was concentrated under reduced pressure to afford crude tert-butyl 8-((2-(3,6-dichloro-1,2,4-triazin-5-yl)-2-azaspiro[3.3]heptan-6-yl)oxy)-3,4-dihydroisoquinoline-2(1H)-carboxylate (6.6 g, 65.8% yield) as a gummy solid. This material was used without further purification. ¹H NMR (400 MHz, DMSO-d₆): δ 7.12 (t, J=7.8 Hz, 1H), 6.75 (br d, J=7.6 Hz, 1H), 6.66 (d, J=8.1 Hz, 1H), 4.83-4.64 (m, 3H), 4.37 (br s, 2H), 4.27-4.16 (m, 2H), 3.53 (br t, J=5.5 Hz, 2H), 2.86-2.78 (m, 2H), 2.73 (br t, J=5.4 Hz, 2H), 2.37-2.21 (m, 2H), 1.44 (s, 9H); LCMS (Method B): Rt=2.43 min, m/z=492.2 [M+H]⁺, 81.34%.

### Step 2. tert-Butyl 8-((2-(3-chloro-6-(2-(((1r,3r)-3-(dimethylamino)-3-methylcyclobutyl)(isopropyl)carbamoyl)-4-fluorophenoxy)-1,2,4-triazin-5-yl)-2-azaspiro[3.3]heptan-6-yl)oxy)-3,4-dihydroisoquinoline-2(1H)-carboxylate and tert-butyl 8-((2-(6-chloro-3-(2-(((1r,3r)-3-(dimethylamino)-3-methylcyclobutyl)(isopropyl)carbamoyl)-4-fluorophenoxy)-1,2,4-triazin-S-yl)-2-azaspiro[3.3]heptan-6-yl)oxy)-3,4-dihydroisoquinoline-2(1H)-carboxylate

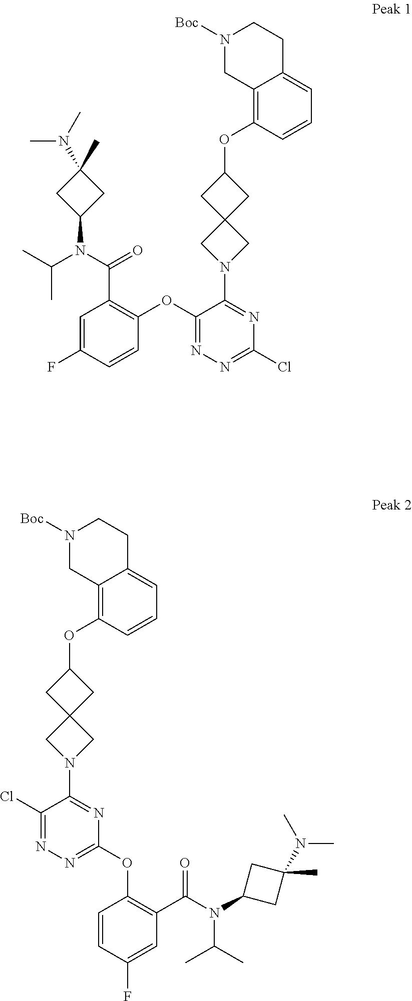 [SMILES: CC(C)N(C(=O)c1cc(F)ccc1Oc1nnc(Cl)nc1N1CC2(CC(Oc3cccc4c3CN(C(=O)OC(C)(C)C)CC4)C2)C1)[C@H]1C[C@](C)(N(C)C)C1 ; CC(C)N(C(=O)c1cc(F)ccc1Oc1nnc(Cl)c(N2CC3(CC(Oc4cccc5c4CN(C(=O)OC(C)(C)C)CC5)C3)C2)n1)[C@H]1C[C@](C)(N(C)C)C1]

In a dried, 250 mL three neck round bottom flask under a nitrogen atmosphere, tert-butyl 8-((2-(3,6-dichloro-1,2,4-triazin-5-yl)-2-azaspiro[3.3]heptan-6-yl)oxy)-3,4-dihydroisoquinoline-2(1H)-carboxylate (3.5 g, 7.11 mmol) and N-((1r,3r)-3-(dimethylamino)-3-methylcyclobutyl)-5-fluoro-2-hydroxy-N-isopropylbenzamide (2.192 g, 7.11 mmol) were dissolved in CPME (35 mL). To this reaction mixture, 1,1,3,3-tetramethylguanidine (2.456 g, 21.32 mmol) was added at RT. The reaction was stirred at 80° C. for 16 h. The reaction progress was monitored by TLC (100% EtOAc) and LCMS. After completion, the reaction was diluted with water (100 mL) and the mixture was extracted with 10% MeOH in DCM (3×100 mL). The combined organic layer was washed with brine (100 mL), dried over anhydrous sodium sulfate, and filtered, and the filtrate was concentrated under reduced pressure. The crude product was purified by silica gel flash column chromatography using EtOAc in hexane (product eluted at 100% EtOAc) to obtain the mixture of regioisomers. The mixture of regioisomers were further purified by reverse phase chromatography using a SiliCycle SiliaSep C18 column (100 g) on Biotage isolera one (Mobile phase A: 10 mM NH₄HCO₃ in water, B: ACN) to obtain tert-butyl 8-((2-(3-chloro-6-(2-(((1r,3r)-3-(dimethylamino)-3-methylcyclobutyl)(isopropyl)carbamoyl)-4-fluorophenoxy)-1,2,4-triazin-5-yl)-2-azaspiro[3.3]heptan-6-yl)oxy)-3,4-dihydroisoquinoline-2(1H)-carboxylate (0.85 g, 0.17% yield) as a solid. LCMS (Method B): Rt=2.02-2.12 min, m/z=764.4 [M+H]⁺, 95.05%; HPLC (Method C): Rt=13.54-13.62 min, 88.15%; Achiral SFC (Method B): Rt=1.82 min, 80.29%.

Note. Undesired regioisomer was not isolated.

### Step 3. tert-Butyl 8-((2-(6-(2-(((1r,3r)-3-(dimethylamino)-3-methylcyclobutyl)(isopropyl)carbamoyl)-4-fluorophenoxy)-1,2,4-triazin-5-yl)-2-azaspiro[3.3]heptan-6-yl)oxy)-3,4-dihydroisoquinoline-2(1H)-carboxylate

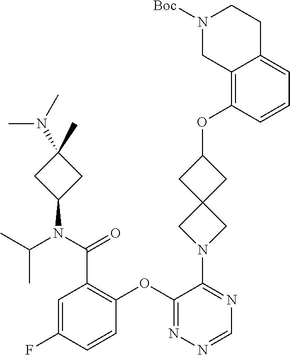 [SMILES: CC(C)N(C(=O)c1cc(F)ccc1Oc1nncnc1N1CC2(CC(Oc3cccc4c3CN(C(=O)OC(C)(C)C)CC4)C2)C1)[C@H]1C[C@](C)(N(C)C)C1]

This compound was synthesized following the procedure described for the synthesis of Example 59, step 5, starting with 1 equivalent of tert-butyl 8-((2-(3-chloro-6-(2-(((1r,3r)-3-(dimethylamino)-3-methylcyclobutyl)(isopropyl)carbamoyl)-4-fluorophenoxy)-1,2,4-triazin-5-yl)-2-azaspiro[3.3]heptan-6-yl)oxy)-3,4-dihydroisoquinoline-2(1H)-carboxylate, except that 0.75 equivalent of Pd—C was used. After completion, the reaction was diluted with MeOH and filtered through a Celite® pad, and the pad was washed with 10% MeOH in DCM. The filtrate was concentrated under reduced pressure. The crude product was reconcentrated from EtOAc to afford crude tert-butyl 8-((2-(6-(2-(((1r,3r)-3-(dimethylamino)-3-methylcyclobutyl)isopropyl)carbamoyl)-4-fluorophenoxy)-1,2,4-triazin-5-yl)-2-azaspiro[3.3]heptan-6-yl)oxy)-3,4-dihydroisoquinoline-2(1H)-carboxylate as a solid. This material was used without further purification.

Yield: 81%; LCMS (Method A): Rt=2.52 min, m/z=730.3 [M+H]⁺; HPLC (Method C): Rt=12.95 min, 90.61%.

### Step 4. N-((1r,3r)-3-(Dimethylamino)-3-methylcyclobutyl)-5-fluoro-N-isopropyl-2-((5-(6-((1,2,3,4-tetrahydroisoquinolin-8-yl)oxy)-2-azaspiro[3.3]heptan-2-yl)-1,2,4-triazin-6-yl)oxy)benzamide

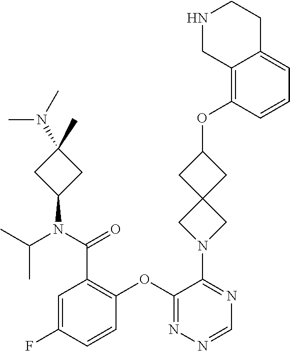 [SMILES: CC(C)N(C(=O)c1cc(F)ccc1Oc1nncnc1N1CC2(CC(Oc3cccc4c3CNCC4)C2)C1)[C@H]1C[C@](C)(N(C)C)C1]

To a stirred solution of tert-butyl 8-((2-(6-(2-(((1r,3r)-3-(dimethylamino)-3-methylcyclobutyl)(isopropyl)carbamoyl)-4-fluorophenoxy)-1,2,4-triazin-5-yl)-2-azaspiro[3.3]heptan-6-yl)oxy)-3,4-dihydroisoquinoline-2(1H)-carboxylate (0.93 g, 1.274 mmol) in TFE (15 mL), TMSCl (0.651 mL, 5.10 mmol) was added at 0° C. The reaction was stirred at RT for 1 h. After completion, the reaction was concentrated under reduced pressure. The crude was quenched with aqueous sodium bicarbonate solution (30 mL) and the mixture was extracted with 10% MeOH in DCM (3×30 mL). The combined organic layer was dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford N-((1r,3r)-3-(dimethylamino)-3-methylcyclobutyl)-5-fluoro-N-isopropyl-2-((5-(6-((1,2,3,4-tetrahydroisoquinolin-8-yl)oxy)-2-azaspiro[3.3]heptan-2-yl)-1,2,4-triazin-6-yl)oxy)benzamide (95 mg, 11.67% yield) as a solid. ¹HNMR (400 MHz, DMSO-d₆): δ 8.46 (s, 1H), 7.51-7.11 (m, 3H), 7.07-6.97 (m, 1H), 6.65 (d, J=7.6 Hz, 1H), 6.61-6.50 (m, 1H), 4.72-4.47 (m, 3H), 4.32-4.06 (m, 2H), 4.00-3.85 (m, 1H), 3.71 (s, 2H), 3.66-3.52 (m, 1H), 2.88 (br t, J=5.6 Hz, 2H), 2.84-2.74 (m, 2H), 2.64 (br t, J=5.2 Hz, 2H), 2.32-2.20 (m, 2H), 2.18-1.91 (m, 8H), 1.84-1.48 (m, 2H), 1.46-0.56 (m, 10H); LCMS (Method A): Rt=1.75 min, m/z=630.2 [M+H]⁺, 95.05%; HPLC (Method C): Rt=9.00 min, 98.58%; Chiral SFC (Method Y): Rt=4.19 min, 100%.

### Example 165. 5-Fluoro-N-isopropyl-N-((1r,3r)-3-methyl-3-(pyrrolidin-1-yl)cyclobutyl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide (Compound No. 165)

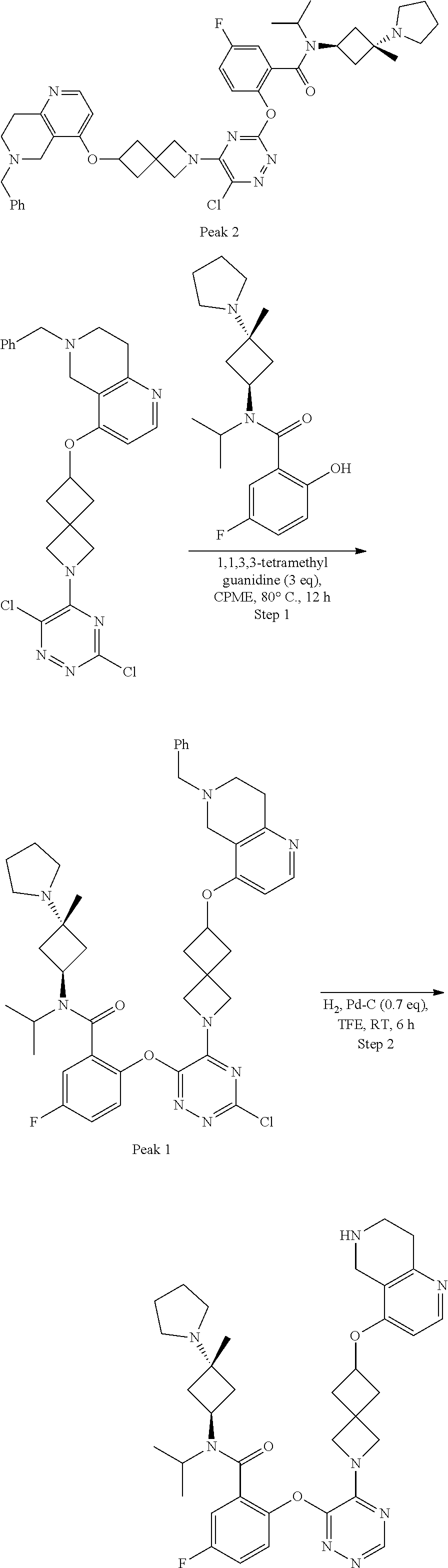

### Step 1. 2-((5-(6-((6-Benzyl-5,6,7,8-tetrahydro-, 6-naphthyridin-4-yl)oxy)-2-azaspiro[3.3]heptan-2-yl)-3-chloro-1,2,4-triazin-6-yl)oxy)-5-fluoro-N-isopropyl-N-((1r,3r)-3-methyl-3-(pyrrolidin-1-yl)cyclobutyl)benzamide and 2-((5-(6-((6-benzyl-5,6,7,8-tetrahydro-1,6-naphthyridin-4-yl)oxy)-2-azaspiro[3.3]heptan-2-yl)-6-chloro-1,2,4-triazin-3-yl)oxy)-5-fluoro-N-isopropyl-N-((1r,3r)-3-methyl-3-(pyrrolidin-1-yl)cyclobutyl)benzamide

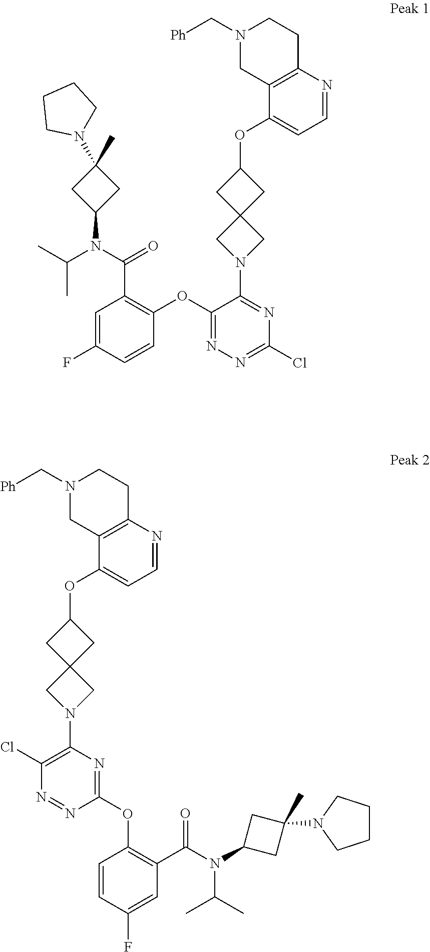 [SMILES: CC(C)N(C(=O)c1cc(F)ccc1Oc1nnc(Cl)nc1N1CC2(CC(Oc3ccnc4c3CN(Cc3ccccc3)CC4)C2)C1)[C@H]1C[C@](C)(N2CCCC2)C1 ; CC(C)N(C(=O)c1cc(F)ccc1Oc1nnc(Cl)c(N2CC3(CC(Oc4ccnc5c4CN(Cc4ccccc4)CC5)C3)C2)n1)[C@H]1C[C@](C)(N2CCCC2)C1]

This compound was synthesized following the procedure described for the synthesis of Example 164, Step 4, starting with 1 equivalent of 6-benzyl-4-((2-(3,6-dichloro-1,2,4-triazin-5-yl)-2-azaspiro[3.3]heptan-6-yl)oxy)-5,6,7,8-tetrahydro-1,6-naphthyridine and 1 equivalent of 5-fluoro-2-hydroxy-N-isopropyl-N-((1r,3r)-3-methyl-3-(pyrrolidin-1-yl)cyclobutyl)benzamide. After work-up, the crude was further purified by reverse phase chromatography using a SiliCycle SiliaSep C18 column (100 g) on Biotage isolera one (Mobile phase A: 10 mM NH₄HCO₃ in water, B: ACN) to obtain 2-((3-chloro-5-(6-((5,6,7,8-tetrahydro-1,6-naphthyridin-4-yl)oxy)-2-azaspiro[3.3]heptan-2-yl)-1,2,4-triazin-6-yl)oxy)-5-fluoro-N-isopropyl-N-((1r,3r)-3-methyl-3-(pyrrolidin-1-yl)cyclobutyl)benzamide as a solid.

Yield: 8.57%; ¹H NMR (400 MHz, DMSO-d₆): δ 8.26-8.15 (m, 1H), 7.52-7.12 (m, 8H), 6.73-6.59 (m, 1H), 4.86-4.48 (m, 3H), 4.38-4.13 (m, 2H), 3.99-3.75 (m, 1H), 3.73-3.62 (m, 3H), 3.47 (s, 2H), 2.91-2.65 (m, 8H), 2.38-2.12 (m, 6H), 1.86-1.75 (m, 1H), 1.72-1.55 (m, 4H), 1.47-0.87 (m, 9H), one proton merged with solvent peaks; LCMS (Method A): Rt=2.33 min, m/z=781.2 [M+H]⁺, 98.52%.

Note. Undesired regioisomer was not isolated.

### Step 2. 5-Fluoro-N-isopropyl-N-((1r,3r)-3-methyl-3-(pyrrolidin-1-yl)cyclobutyl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide

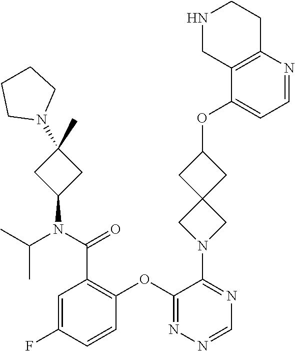 [SMILES: CC(C)N(C(=O)c1cc(F)ccc1Oc1nncnc1N1CC2(CC(Oc3ccnc4c3CNCC4)C2)C1)[C@H]1C[C@](C)(N2CCCC2)C1]

This compound was synthesized following the procedure described for the synthesis of Example 59, step 5, starting with 1 equivalent of 2-((5-(6-((6-benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-((1r,3r)-3-methyl-3-(pyrrolidin-1-yl)cyclobutyl)benzamide, except that 0.7 equivalent of Pd—C was used. After filtration, the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain 5-fluoro-N-isopropyl-N-((1r,3r)-3-methyl-3-(pyrrolidin-1-yl)cyclobutyl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide as a solid.

Yield: 41.7%; ¹H NMR (400 MHz, DMSO-d₆): δ 8.46 (s, 1H), 8.17 (br d, J=4.6 Hz, 1H), 7.53-7.10 (m, 3H), 6.72-6.58 (m, 1H), 4.82-4.74 (m, 1H), 4.71-4.49 (m, 2H), 4.36-4.06 (m, 2H), 4.02-3.78 (m, 1H), 3.71 (s, 2H), 3.66-3.52 (m, 1H), 2.96 (br t, J=5.6 Hz, 2H), 2.89-2.77 (m, 3H), 2.71-2.67 (m, 2H), 2.39-2.27 (m, 4H), 2.17 (br s, 2H), 1.87-1.74 (m, 1H), 1.72-1.53 (m, 5H), 1.48-0.53 (m, 9H), two protons merged with solvent peaks; LCMS (Method H): Rt=1.04 min, m/z=657.3 [M+H]⁺; HPLC (Method G): Rt=6.34 min, 98.51%; Chiral SFC (Method Q): Rt=4.62 min, 100%.

### Example 166. (D1)-5-Fluoro-2-((5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-isopropyl-N-((1r,3r)-3-methyl-3-(pyrrolidin-1-yl)cyclobutyl)benzamide (Compound No. 166)

### Example 167. (D2)-5-Fluoro-2-((5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-isopropyl-N-((1r,3r)-3-methyl-3-(pyrrolidin-1-yl)cyclobutyl)benzamide (Compound No. 167)

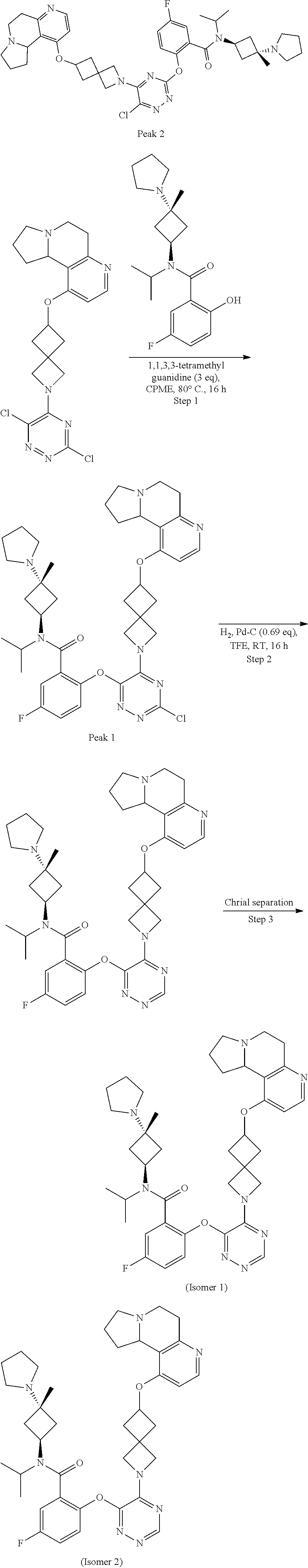

### Step 1. 2-((3-Chloro-5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-5-fluoro-N-isopropyl-N-((1r,3r)-3-methyl-3-(pyrrolidin-1-yl)cyclobutyl)benzamide and 2-((6-chloro-5-(6-((5,6,8,9,10,10α-hexahydropyrrolo[2,1-f][1,6]naphthyridin-1-yl)oxy)-2-azaspiro[3.3]heptan-2-yl)-1,2,4-triazin-3-yl)oxy)-5-fluoro-N-isopropyl-N-((1r,3r)-3-methyl-3-(pyrrolidin-1-yl)cyclobutyl)benzamide

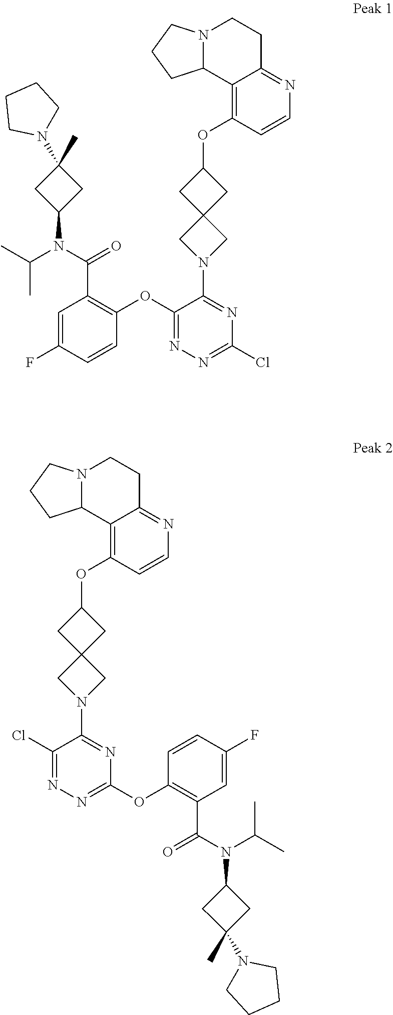 [SMILES: CC(C)N(C(=O)c1cc(F)ccc1Oc1nnc(Cl)nc1N1CC2(CC(Oc3ccnc4c3C3CCCN3CC4)C2)C1)[C@H]1C[C@](C)(N2CCCC2)C1 ; CC(C)N(C(=O)c1cc(F)ccc1Oc1nnc(Cl)c(N2CC3(CC(Oc4ccnc5c4C4CCCN4CC5)C3)C2)n1)[C@H]1C[C@](C)(N2CCCC2)C1]

This compound was synthesized following the procedure described for the synthesis of Example 164, step 4, starting with 1 equivalent of (±)-1-((2-(3,6-dichloro-1,2,4-triazin-5-yl)-2-azaspiro[3.3]heptan-6-yl)oxy)-5,6,8,9,10,10a-hexahydropyrrolo[2,1-f][1,6]naphthyridine and 1 equivalent of 5-fluoro-2-hydroxy-N-isopropyl-N-((1r,3r)-3-methyl-3-(pyrrolidin-1-yl)cyclobutyl)benzamide. The reaction was diluted with water and the mixture was extracted with EtOAc. The combined organic layer was dried over anhydrous sodium sulfate and filtered, and the filtrate was concentrated under reduced pressure. This mixture was purified by achiral Prep SFC (Method E). The pure fractions were concentrated under reduced pressure and re-concentrated from ACN to afford both regioisomers.

Peak 1. 2-((3-Chloro-5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-5-fluoro-N-isopropyl-N-((1r,3r)-3-methyl-3-(pyrrolidin-1-yl)cyclobutyl)benzamide (1 g, 13.98% yield) as a solid. LCMS (Method C): Rt=1.32 min, m/z=731.3 [M+H]⁺; HPLC (Method C): Rt=9.17 min, 97.85%; Achiral SFC (Method C): Rt=4.62 min, 99.42%.

Peak 2. 2-((6-Chloro-5-(6-((5,6,8,9,10,10a-hexahydropyrrolo[2,1-f][1,6]naphthyridin-1-yl)oxy)-2-azaspiro[3.3]heptan-2-yl)-1,2,4-triazin-3-yl)oxy)-5-fluoro-N-isopropyl-N-((1r,3r)-3-methyl-3-(pyrrolidin-1-yl)cyclobutyl)benzamide (180 mg, 2.20% yield) as a solid. LCMS (Method C): Rt=1.36 min, m/z=731.3 [M+H]⁺; HPLC (Method G): Rt=7.24 min, 85.59%; Achiral SFC (Method C): Rt=6.08 min, 77.48%.

### Step 2. 5-Fluoro-2-((5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-N-isopropyl-N-((1r,3r)-3-methyl-3-(pyrrolidin-1-yl)cyclobutyl)benzamide

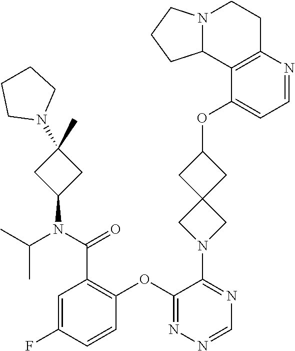 [SMILES: CC(C)N(C(=O)c1cc(F)ccc1Oc1nncnc1N1CC2(CC(Oc3ccnc4c3C3CCCN3CC4)C2)C1)[C@H]1C[C@](C)(N2CCCC2)C1]

This compound was synthesized following the procedure described for the synthesis of Example 59, step 5, starting with 1 equivalent of 2-((3-chloro-5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-5-fluoro-N-isopropyl-N-((1r,3r)-3-methyl-3-(pyrrolidin-1-yl)cyclobutyl)benzamide, except that 0.69 equivalent of Pd—C was used.

After completion, the reaction was filtered through a Celite® pad, and the pad was washed with MeOH. The filtrate was concentrated under reduced pressure. The crude was dissolved in 10% MeOH in DCM and washed with 10% NaHCO₃ solution. The combined organic layer was dried over anhydrous sodium sulfate and filtered, and the filtrate was concentrated under reduced pressure to afford 5-fluoro-2-((5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-isopropyl-N-((1r,3r)-3-methyl-3-(pyrrolidin-1-yl)cyclobutyl)benzamide as a solid.

Yield: 65.8%; ¹H NMR (400 MHz, DMSO-d₆): δ 8.46 (s, 1H), 8.21-8.13 (m, 1H), 7.50-7.13 (m, 3H), 6.68 (br s, 1H), 4.87-4.76 (m, 1H), 4.73-4.51 (m, 2H), 4.31-3.95 (m, 3H), 3.81-3.50 (m, 3H), 3.01-2.61 (m, 9H), 2.43-2.10 (m, 6H), 1.87-1.37 (m, 11H), 1.33-0.55 (m, 7H); LCMS (Method C): Rt=1.17 min, m/z=697.3 [M+H]⁺; HPLC (Method G): Rt=6.48 min, 98.04%; Chiral SFC (Method AJ): Peak 1: Rt=3.42 min, 49.98%; Peak 2: Rt=5.01 min, 50.02%.

### Step 3. (D1)-5-Fluoro-2-((5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-N-isopropyl-N-((1r,3r)-3-methyl-3-(pyrrolidin-1-yl)cyclobutyl)benzamide and (D2)-5-fluoro-2-((5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-N-isopropyl-N-((1r,3r)-3-methyl-3-(pyrrolidin-1-yl)cyclobutyl)benzamide

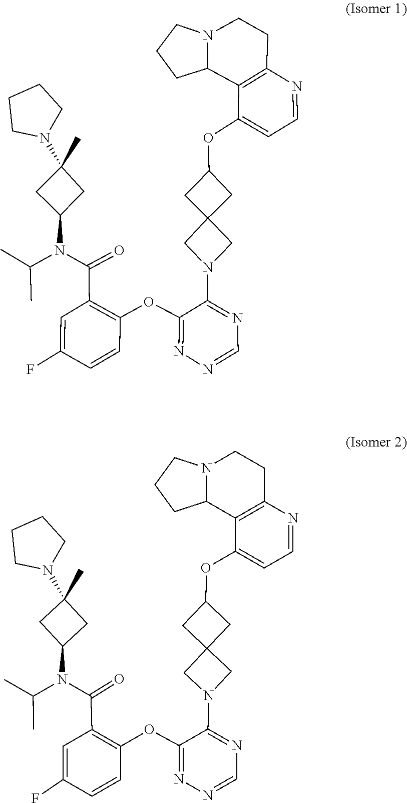 [SMILES: CC(C)N(C(=O)c1cc(F)ccc1Oc1nncnc1N1CC2(CC(Oc3ccnc4c3C3CCCN3CC4)C2)C1)[C@H]1C[C@](C)(N2CCCC2)C1 ; CC(C)N(C(=O)c1cc(F)ccc1Oc1nncnc1N1CC2(CC(Oc3ccnc4c3C3CCCN3CC4)C2)C1)[C@H]1C[C@](C)(N2CCCC2)C1]

The diastereomeric mixture of 5-fluoro-2-((5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-isopropyl-N-((1r,3r)-3-methyl-3-(pyrrolidin-1-yl)cyclobutyl)benzamide (640 mg, 0.918 mmol) was purified by chiral Prep SFC (Method A). The pure fractions were concentrated under reduced pressure, re-concentrated from ACN, then lyophilized to obtain (D1)-5-fluoro-2-((5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-isopropyl-N-((1r,3r)-3-methyl-3-(pyrrolidin-1-yl)cyclobutyl)benzamide (203.37 mg, 31.6% yield) and (D2)-5-fluoro-2-((5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-isopropyl-N-((1r,3r)-3-methyl-3-(pyrrolidin-1-yl)cyclobutyl)benzamide (203.61 mg, 31.8% yield) as solids.

Isomer 1. ¹H NMR (400 MHz, DMSO-d₆): δ 8.46 (s, 1H), 8.18 (br d, J=5.0 Hz, 1H), 7.48-7.07 (m, 3H), 6.68 (br s, 1H), 4.84-4.75 (m, 1H), 4.73-4.49 (m, 2H), 4.31-3.94 (m, 3H), 3.86-3.51 (m, 3H), 3.04-2.60 (m, 9H), 2.45-2.06 (m, 6H), 1.88-1.36 (m, 11H), 1.33-0.54 (m, 7H); LCMS (Method C): Rt=1.15 min, m/z=697.4 [M+H]⁺; HPLC (Method G): Rt=6.52 min, 99.46%; Chiral SFC (Method AJ): Rt=3.45 min, 100%.

Isomer 2. ¹H NMR (400 MHz, DMSO-d₆): δ 8.46 (s, 1H), 8.18 (br d, J=5.4 Hz, 1H), 7.50-7.10 (m, 3H), 6.68 (br s, 1H), 4.86-4.76 (m, 1H), 4.72-4.47 (m, 2H), 4.27-3.93 (m, 3H), 3.81-3.54 (m, 3H), 3.04-2.63 (m, 9H), 2.45-2.09 (m, 6H), 1.88-1.38 (m, 11H), 1.34-0.56 (m, 7H); LCMS (Method C): Rt=1.15 min, m/z=697.4 [M+H]⁺; HPLC (Method G): Rt=6.52 min, 99.81%; Chiral SFC (Method AJ): Rt=5.25 min, 99.17%.

### Example 168. N-((1r,3r)-3-(Dimethylamino)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyridazin-3-yl)oxy)benzamide (Compound No. 168)

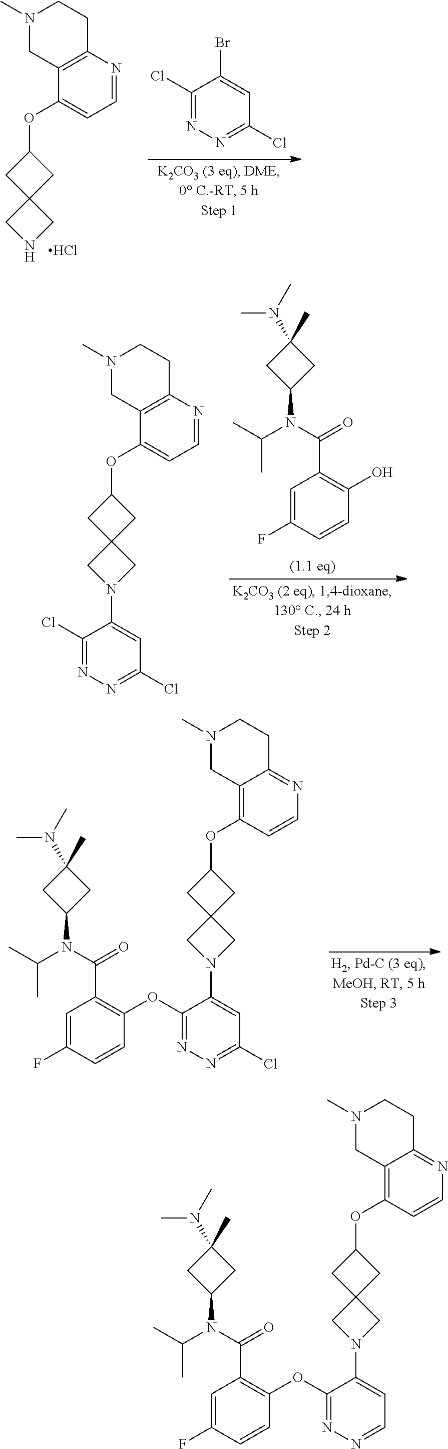

### Step 1. 4-((2-(3,6-Dichloropyridazin-4-yl)-2-azaspiro[3.3]heptan-6-yl)oxy)-6-methyl-5,6,7,8-tetrahydro-1,6-naphthyridine

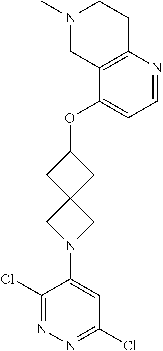 [SMILES: CN1CCc2nccc(OC3CC4(C3)CN(c3cc(Cl)nnc3Cl)C4)c2C1]

This compound was synthesized following the procedure described for the synthesis of Intermediate 34, starting with 1 equivalent of 4-((2-azaspiro[3.3]heptan-6-yl)oxy)-6-methyl-5,6,7,8-tetrahydro-1,6-naphthyridine hydrochloride and 1 equivalent of 4-bromo-3,6-dichloropyridazine, except that 3 equivalents of K₂CO₃ was used, and DME was used as the solvent. After work-up, the crude was purified by silica gel flash column chromatography using MeOH in DCM (product eluted at 16% MeOH in DCM) to obtain 4-((2-(3,6-dichloropyridazin-4-yl)-2-azaspiro[3.3]heptan-6-yl)oxy)-6-methyl-5,6,7,8-tetrahydro-1,6-naphthyridine as a solid.

Yield: 9.94%; ¹H NMR (400 MHz, DMSO-d₆): δ 8.20 (d, J=5.6 Hz, 1H), 6.72 (s, 1H), 6.68 (d, J=5.8 Hz, 1H), 4.77 (quin, J=6.7 Hz, 1H), 4.40 (br s, 2H), 4.30 (br s, 2H), 3.39 (s, 2H), 2.89-2.78 (m, 4H), 2.68-2.61 (m, 2H), 2.39 (s, 3H), 2.35-2.26 (in, 2H); LCMS (Method A): Rt=1.64 min m/z=406.0 [M+H]⁺, 95.85%.

### Step 2. 2-((6-Chloro-4-(6-((6-methyl-5,6,7,8-tetrahydro-1,6-naphthyridin-4-yl)oxy)-2-azaspiro[3.3]heptan-2-yl)pyridazin-3-yl)oxy)-N-((1r,3r)-3-(dimethylamino)-3-methylcyclobutyl)-5-fluoro-N-isopropylbenzamide

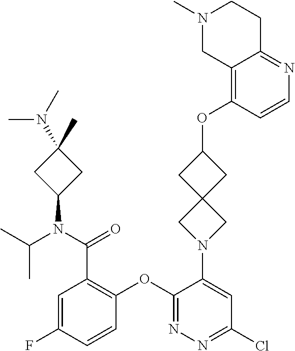 [SMILES: CC(C)N(C(=O)c1cc(F)ccc1Oc1nnc(Cl)cc1N1CC2(CC(Oc3ccnc4c3CN(C)CC4)C2)C1)[C@H]1C[C@](C)(N(C)C)C1]

This compound was synthesized following the procedure described for the synthesis of Example 59, step 4, starting with 1 equivalent of 4-((2-(3,6-dichloropyridazin-4-yl)-2-azaspiro[3.3]heptan-6-yl)oxy)-6-methyl-5,6,7,8-tetrahydro-1,6-naphthyridine and 1.1 equivalents of N-((1r,3r)-3-(dimethylamino)-3-methylcyclobutyl)-5-fluoro-2-hydroxy-N-isopropylbenzamide, except that 2 equivalents of K₂CO₃ was used and 1,4-dioxane was used as the solvent. The reaction was stirred at 130° C. After completion, the reaction was concentrated under reduced pressure. The crude product was purified by reverse phase chromatography using a SiliCycle SiliaSep C18 column (80 g) on Biotage isolera one (Mobile phase A: 10 mM NH₄HCO₃ in water, B: ACN) to obtain 2-((6-chloro-4-(6-((6-methyl-5,6,7,8-tetrahydro-1,6-naphthyridin-4-yl)oxy)-2-azaspiro[3.3]heptan-2-yl)pyridazin-3-yl)oxy)-N-((1r,3r)-3-(dimethylamino)-3-methylcyclobutyl)-5-fluoro-N-isopropylbenzamide as a solid.

Yield: 41.4%; ¹H NMR (400 MHz, DMSO-d₆): δ 8.19 (br d, J=5.1 Hz, 1H), 7.44-7.07 (m, 3H), 6.71-6.62 (m, 1H), 6.60-6.50 (m, 1H), 4.89-4.68 (m, 1H), 4.50-4.15 (m, 3H), 3.98-3.54 (m, 3H), 3.37 (s, 2H), 2.90-2.73 (m, 5H), 2.66-2.61 (m, 2H), 2.37 (s, 3H), 2.32-2.24 (m, 2H), 2.19-2.02 (m, 5H), 1.95 (s, 3H), 1.78-1.47 (m, 1H), 1.46-0.84 (m, 9H); LCMS (Method A): Rt=1.94 min, m/z=678.2 [M+H]⁺, 99.46%.

### Step 3. N-((1r,3r)-3-(Dimethylamino)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyridazin-3-yl)oxy)benzamide

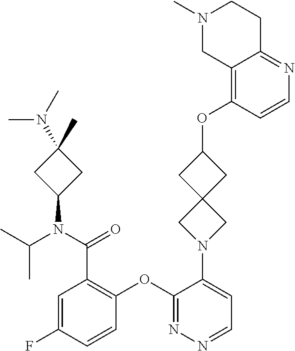 [SMILES: CC(C)N(C(=O)c1cc(F)ccc1Oc1nnccc1N1CC2(CC(Oc3ccnc4c3CN(C)CC4)C2)C1)[C@H]1C[C@](C)(N(C)C)C1]

This compound was synthesized following the procedure described for the synthesis of Example 59, step 4, starting with 1 equivalent of 2-((6-chloro-4-(6-((6-methyl-5,6,7,8-tetrahydro-1,6-naphthyridin-4-yl)oxy)-2-azaspiro[3.3]heptan-2-yl)pyridazin-3-yl)oxy)-N-((1r,3r)-3-(dimethylamino)-3-methylcyclobutyl)-5-fluoro-N-isopropylbenzamide, except that 0.6 equivalents of Pd—C was used. After filtration and concentration, the crude was purified by reverse phase chromatography using a SiliCycle SiliaSep C18 column (80 g) on Biotage isolera one (Mobile phase A: 10 mM NH₄HCO₃ in water, B: ACN), then by chiral Prep SFC (Method C) to obtain N-((1r,3r)-3-(dimethylamino)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyridazin-3-yl)oxy)benzamide as a solid.

Yield: 42.15%; ¹H NMR (400 MHz, DMSO-d₆): δ 8.41 (d, J=5.4 Hz, 1H), 8.19 (br d, J=5.4 Hz, 1H), 7.42-7.23 (m, 3H), 6.72-6.63 (m, 1H), 6.50-6.40 (m, 1H), 4.86-4.71 (m, 1H), 4.43-3.95 (m, 5H), 3.79-3.55 (m, 2H), 3.38 (s, 2H), 2.88-2.73 (m, 5H), 2.67-2.61 (m, 2H), 2.37 (s, 3H), 2.32-2.24 (m, 2H), 2.21-2.03 (m, 4H), 1.95 (br s, 2H), 1.82-1.66 (m, 1H), 1.44-0.88 (m, 9H), one proton merged with solvent peaks: LCMS (Method A): Rt=1.78 min, m/z=644.2 [M+H]⁺; HPLC (Method C): Rt=8.43 min, 99.41%; Chiral SFC (Method E): Rt=3.31 min, 100%.

### Example 169. N-((1r,3r)-3-(Azetidin-1-yl)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 169)

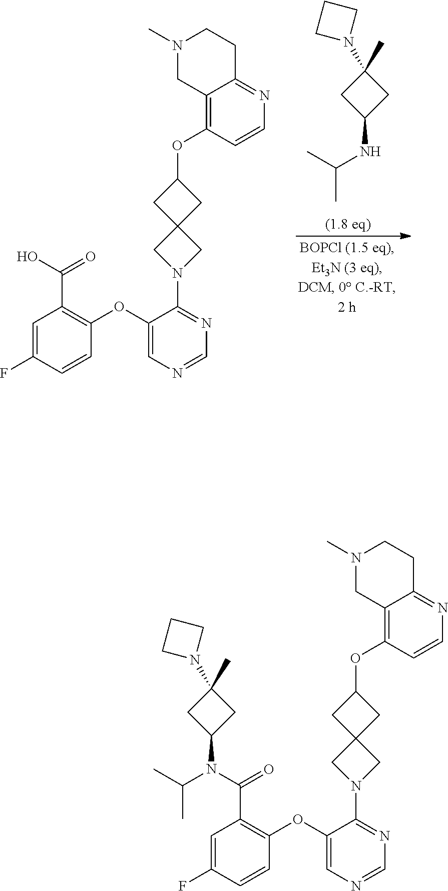 [SMILES: CN1CCc2nccc(OC3CC4(C3)CN(c3ncncc3Oc3ccc(F)cc3C(=O)O)C4)c2C1 ; CC(C)N[C@H]1C[C@](C)(N2CCC2)C1 ; CC(C)N(C(=O)c1cc(F)ccc1Oc1cncnc1N1CC2(CC(Oc3ccnc4c3CN(C)CC4)C2)C1)[C@H]1C[C@](C)(N2CCC2)C1]

To a stirred solution of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid (60 mg, 0.122 mmol) in DCM (3 mL), (1r,3r)-3-(azetidin-1-yl)-N-isopropyl-3-methylcyclobutan-1-amine (40 mg, 0.219 mmol), Et₃N (0.051 mL, 0.366 mmol), and BOP-Cl (46.6 mg, 0.183 mmol) were added at 0° C. The reaction was stirred at RT for 2 h. The reaction progress was monitored by TLC (10% MeOH in DCM). After completion, the reaction was quenched with ice-cold water (10 mL), and the mixture was extracted with DCM (2×20 mL). The combined organic layer was washed with brine solution (15 mL), dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain N-((1r,3r)-3-(azetidin-1-yl)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (20 mg, 19.34% yield) as a solid. ¹HNMR (400 MHz, DMSO-d₆): δ 8.32-8.25 (m, 1H), 8.18 (d, J=5.6 Hz, 1H), 7.80-7.59 (m, 1H), 7.34-7.03 (m, 3H), 6.69-6.61 (m, 1H), 4.75 (quin, J=6.7 Hz, 1H), 4.38-4.09 (m, 4H), 3.79-3.62 (m, 2H), 3.36 (s, 2H), 3.11 (br t, J=6.7 Hz, 2H), 2.96-2.88 (m, 2H), 2.85-2.73 (m, 5H), 2.66-2.60 (m, 2H), 2.36 (s, 3H), 2.30-2.21 (m, 1H), 2.19-1.97 (m, 2H), 1.93-1.72 (m, 3H), 1.49-0.87 (m, 9H), one proton merged with solvent peaks; LCMS (Method C): Rt=1.41 min, m/z=656.5 [M+H]⁺; HPLC (Method C): Rt=8.57 min, 96.70%; Chiral SFC (Method J): Rt=7.13 min, 100%.

### Example 170. (D2)-2-((4-(6-((5,6-Dimethyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide (Compound No. 170)

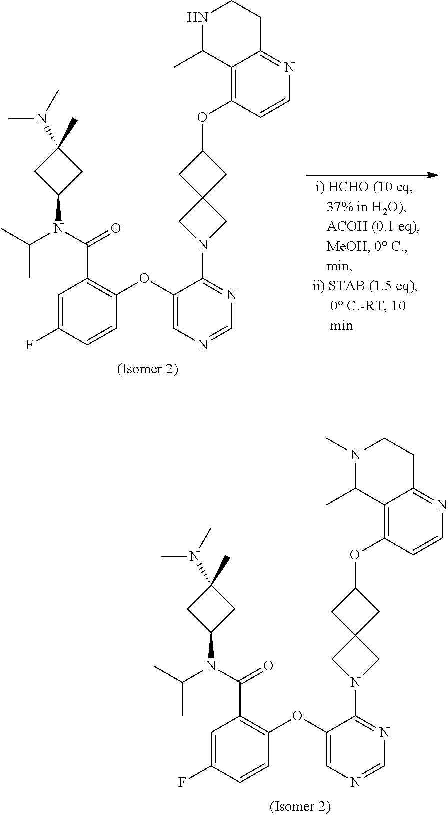 [SMILES: CC1NCCc2nccc(OC3CC4(C3)CN(c3ncncc3Oc3ccc(F)cc3C(=O)N(C(C)C)[C@H]3C[C@](C)(N(C)C)C3)C4)c21 ; CC1c2c(OC3CC4(C3)CN(c3ncncc3Oc3ccc(F)cc3C(=O)N(C(C)C)[C@H]3C[C@](C)(N(C)C)C3)C4)ccnc2CCN1C]

This compound was synthesized following the procedure described for the synthesis of Example 76, starting with 1 equivalent of (D2)-N-((1r,3r)-3-(dimethylamino)-3-methylcyclobutyl)-5-fluoro-N-isopropyl-2-((4-(6-((5-methyl-5,6,7,8-tetrahydro-1,6-naphthyridin-4-yl)oxy)-2-azaspiro[3.3]heptan-2-yl)pyrimidin-5-yl)oxy)benzamide, except that 10 equivalents of formaldehyde (37% in H₂O), 0.1 equivalent of AcOH, and 1.5 equivalents of STAB were used. After completion, the crude was diluted with aqueous NaHCO₃ solution and the mixture was extracted with 10% MeOH in DCM. The combined organic layer dried over anhydrous sodium sulfate and filtered, and the filtrate was concentrated under reduced pressure. The crude product was purified by reverse phase chromatography using a SiliCycle SiliaSep C18 column (40 g) on Biotage isolera one (Mobile phase A: 10 mM NH₄HCO₃ in water, B: ACN) to obtain (D2)-2-((4-(6-((5,6-dimethyl-5,6,7,8-tetrahydro-1,6-naphthyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide as a solid.

Yield: 64.7%; ¹H NMR (400 MHz, DMSO-d₆): δ 8.32-8.25 (m, 1H), 8.17 (d, J=5.4 Hz, 1H), 7.81-7.58 (m, 1H), 7.33-7.04 (m, 3H), 6.69-6.61 (m, 1H), 4.75 (quin, J=6.4 Hz, 1H), 4.44-3.93 (m, 4H), 3.86-3.61 (m, 3H), 3.04-2.93 (m, 1H), 2.92-2.74 (m, 3H), 2.71-2.59 (m, 3H), 2.34 (s, 3H), 2.30-2.19 (m, 2H), 2.17-2.04 (m, 5H), 1.99 (s, 3H), 1.95-1.86 (m, 1H), 1.47-0.91 (m, 12H); LCMS (Method A): Rt=1.82 min, m/z=658.3 [M+H]⁺; HPLC (Method C): Rt=8.49 min, 98.45%; Chiral SFC (Method AS): Rt=2.79 min, 100%.

### Example 171. 5-Fluoro-N-isopropyl-N-((1r,3r)-3-methyl-3-(pyrrolidin-1-yl)cyclobutyl)-2-((4-(6-((6-methyl-6,7-dihydro-5H-pyrrolo[3,4-b]pyridin-4-yl)oxy)-2-azaspiro[3.3]heptan-2-yl)pyrimidin-5-yl)oxy)benzamide (Compound No. 171)

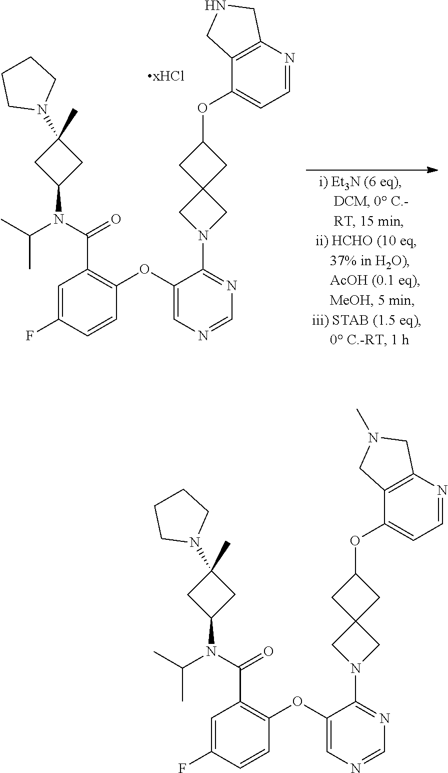 [SMILES: CC(C)N(C(=O)c1cc(F)ccc1Oc1cncnc1N1CC2(CC(Oc3ccnc4c3CNC4)C2)C1)[C@H]1C[C@](C)(N2CCCC2)C1 ; CC(C)N(C(=O)c1cc(F)ccc1Oc1cncnc1N1CC2(CC(Oc3ccnc4c3CN(C)C4)C2)C1)[C@H]1C[C@](C)(N2CCCC2)C1]

This compound was synthesized following the procedure described for the synthesis of Example 20, Step 4, starting with 1 equivalent of 2-((4-(6-((6,7-dihydro-5H-pyrrolo[3,4-b]pyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide xHCl, except that 6 equivalents of TEA, 0.1 equivalent of AcOH, and 1.5 equivalents of STAB were used. After completion, the reaction was quenched with ice-cold water, and aqueous Na₂CO₃ solution was added. The mixture was extracted with EtOAc. The combined organic layer was dried over anhydrous sodium sulfate and filtered, and the filtrate was concentrated under reduced pressure. The crude was purified by Prep HPLC (Method N), then by reverse phase chromatography using a SiliCycle SiliaSep C18 column (40 g) on Biotage isolera one (Mobile phase A: 10 mM NH₄HCO₃ in water, B: ACN) to obtain 5-fluoro-N-isopropyl-N-((1r,3r)-3-methyl-3-(pyrrolidin-1-yl)cyclobutyl)-2-((4-(6-((6-methyl-6,7-dihydro-5H-pyrrolo[3,4-b]pyridin-4-yl)oxy)-2-azaspiro[3.3]heptan-2-yl)pyrimidin-5-yl)oxy)benzamide as a solid.

Yield: 47.2% yield; ¹H NMR (400 MHz, DMSO-d₆): δ8.27 (s, 1H), 8.19 (br d, J=5.6 Hz, 1H), 7.78-7.57 (m, 1H), 7.33-7.03 (m, 3H), 6.69 (br d, J=5.0 Hz, 1H), 4.78 (quin, J=6.6 Hz, 1H), 4.37-4.03 (m, 5H), 3.87-3.77 (m, 4H), 3.75-3.65 (m, 1H), 2.87-2.73 (m, 3H), 2.47 (br s, 3H), 2.42-2.36 (m, 2H), 2.31-2.07 (m, 4H), 2.03-1.91 (m, 1H), 1.85-1.75 (m, 1H), 1.73-1.66 (m, 2H), 1.63-1.56 (m, 2H), 1.49-0.90 (m, 9H), one proton merged with solvent peaks; LCMS (Method H): Rt=1.28 min, m/z=656.4 [M+H]⁺; HPLC (Method C): Rt=8.45 min, 99.30%; Chiral SFC (Method C): Rt=3.31 min, 100%.

### Example 172. N-((1r,3r)-3-(Azetidin-1-yl)-3-methylcyclobutyl)-5-fluoro-N-isopropyl-2-((4-(6-((6-methyl-6,7-dihydro-5H-pyrrolo[3,4-b]pyridin-4-yl)oxy)-2-azaspiro 13.31 heptan-2-yl)pyrimidin-5-yl)oxy)benzamide (Compound No. 172)

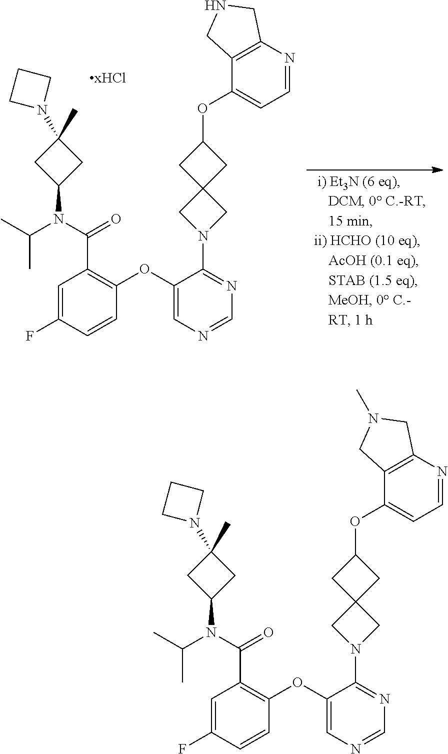 [SMILES: CC(C)N(C(=O)c1cc(F)ccc1Oc1cncnc1N1CC2(CC(Oc3ccnc4c3CNC4)C2)C1)[C@H]1C[C@](C)(N2CCC2)C1 ; CC(C)N(C(=O)c1cc(F)ccc1Oc1cncnc1N1CC2(CC(Oc3ccnc4c3CN(C)C4)C2)C1)[C@H]1C[C@](C)(N2CCC2)C1]

This compound was synthesized following the procedure described for the synthesis of Example 20, step 4, starting with 1 equivalent of N-((1r,3r)-3-(azetidin-1-yl)-3-methylcyclobutyl)-2-((4-(6-((6,7-dihydro-5H-pyrrolo[3,4-b]pyridin-4-yl)oxy)-2-azaspiro[3.3]heptan-2-yl)pyrimidin-5-yl)oxy)-5-fluoro-N-isopropylbenzamide xHCl, except that 6 equivalents of TEA, 0.1 equivalent of AcOH, and 1.5 equivalents of STAB were used. After completion, the reaction was quenched with ice-cold water, and aqueous Na₂CO₃ solution was added. The mixture was extracted with EtOAc. The combined organic layer was dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain N-((1r,3r)-3-(azetidin-1-yl)-3-methylcyclobutyl)-5-fluoro-N-isopropyl-2-((4-(6-((6-methyl-6,7-dihydro-5H-pyrrolo[3,4-b]pyridin-4-yl)oxy)-2-azaspiro[3.3]heptan-2-yl)pyrimidin-5-yl)oxy)benzamide as a solid.

Yield: 29.1%; ¹H NMR (400 MHz, DMSO-d₆): δ 8.29-8.25 (m, 1H), 8.19 (d, J=5.8 Hz, 1H), 7.79-7.61 (m, 1H), 7.35-7.04 (m, 3H), 6.74-6.63 (m, 1H), 4.78 (quin, J=6.3 Hz, 1H), 4.46-4.10 (m, 4H), 3.79 (s, 4H), 3.75-3.65 (m, 1H), 3.11 (br t, J=6.7 Hz, 2H), 2.91 (br t, J=6.6 Hz, 2H), 2.84-2.72 (m, 3H), 2.62-2.56 (m, 1H), 2.47 (s, 2H), 2.32-2.20 (m, 2H), 2.19-1.96 (m, 2H), 1.93-1.70 (m, 3H), 1.50-0.86 (m, 9H), one proton merged with solvent peaks; LCMS (Method A): Rt=1.67 min, m/z=642.2 [M+H]⁺; HPLC (Method C): Rt=8.30 min, 99.36%; Chiral SFC (Method L): Rt=1.53 min, 100%.

### Example 173. N-((1r,3r)-3-(Dimethylamino)-3-methylcyclobutyl)-5-fluoro-N-isopropyl-2-((4-(6-((6-methyl-6,7-dihydro-5H-pyrrolo[3,4-b]pyridin-4-yl)oxy)-2-azaspiro[3.3]heptan-2-yl)pyrimidin-5-yl)oxy)benzamide (Compound No. 173)

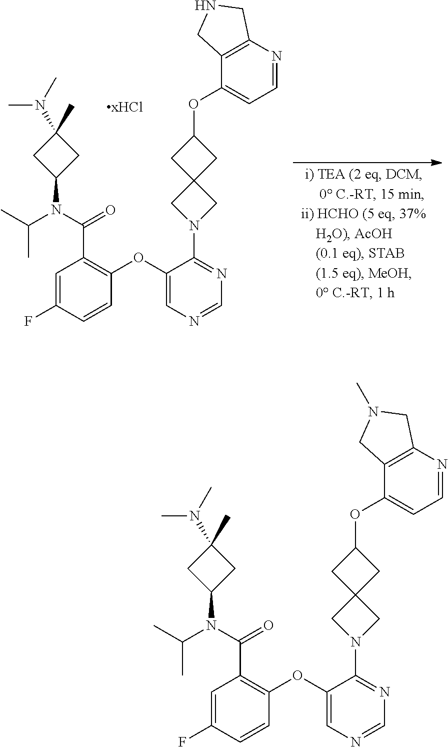 [SMILES: CC(C)N(C(=O)c1cc(F)ccc1Oc1cncnc1N1CC2(CC(Oc3ccnc4c3CNC4)C2)C1)[C@H]1C[C@](C)(N(C)C)C1 ; CC(C)N(C(=O)c1cc(F)ccc1Oc1cncnc1N1CC2(CC(Oc3ccnc4c3CN(C)C4)C2)C1)[C@H]1C[C@](C)(N(C)C)C1]

This compound was synthesized following the procedure described for the synthesis of Example 20, step 4, starting with 1 equivalent of 2-((4-(6-((6,7-dihydro-5H-pyrrolo[3,4-b]pyridin-4-yl)oxy)-2-azaspiro[3.3]heptan-2-yl)pyrimidin-5-yl)oxy)-N-((1r,3r)-3-(dimethylamino)-3-methylcyclobutyl)-5-fluoro-N-isopropylbenzamide xHCl, except that 5 equivalents of formaldehyde (37% in H₂O), 0.1 equivalent of AcOH, and 1.5 equivalents of STAB were used. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain N-((1r,3r)-3-(dimethylamino)-3-methylcyclobutyl)-5-fluoro-N-isopropyl-2-((4-(6-((6-methyl-6,7-dihydro-5H-pyrrolo[3,4-b]pyridin-4-yl)oxy)-2-azaspiro[3.3]heptan-2-yl)pyrimidin-5-yl)oxy)benzamide as a solid.

Yield: 36.2%; ¹H NMR (400 MHz, DMSO-d₆): δ 8.31-8.24 (m, 1H), 8.19 (d, J=5.6 Hz, 1H), 7.80-7.58 (m, 1H), 7.34-7.04 (m, 3H), 6.74-6.65 (m, 1H), 4.78 (quin, J=6.6 Hz, 1H), 4.45-4.11 (m, 4H), 4.05-3.82 (m, 1H), 3.79 (s, 4H), 3.74-3.65 (m, 1H), 2.85-2.73 (m, 3H), 2.47 (s, 3H), 2.31-2.22 (m, 2H), 2.20-1.92 (m, 6H), 1.83-1.67 (m, 1H), 1.51-0.87 (m, 9H); LCMS (Method H): Rt=1.27 min, m/z=630.4 [M+H]⁺; HPLC (Method G): Rt=6.46 min, 98.91%; Chiral SFC (Method AAG): Rt=1.17 min, 100%.

### Example 174. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-methyl-5-azaspiro[3.4]octan-2-yl)benzamide (Compound No. 174)

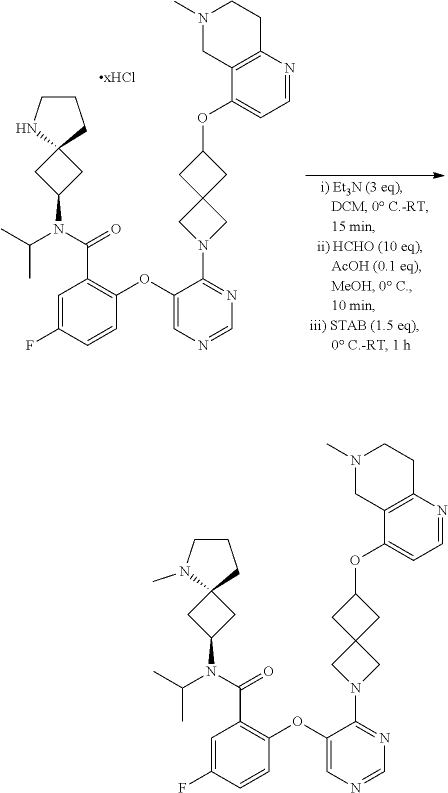 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCN2)C1 ; CC(C)N(C(=O)c1cc(F)ccc1Oc1cncnc1N1CC2(CC(Oc3ccnc4c3CN(C)CC4)C2)C1)[C@H]1C[C@@]2(CCCN2C)C1]

This compound was synthesized following the procedure described for the synthesis of Example 20, step 4, starting with 1 equivalent of 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-azaspiro[3.4]octan-2-yl)benzamide xHCl, except that 3 equivalents of Et₃N, 0.1 equivalent of AcOH, and 1.5 equivalents of STAB were used. After work-up, the crude was purified by reverse phase chromatography using a SiliCycle SiliaSep C18 column (50 g) on Biotage isolera one (Mobile phase A: 10 mM NH₄HCO₃ in water, B: ACN) to obtain 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-methyl-5-azaspiro[3.4]octan-2-yl)benzamide as a solid.

Yield: 17.34%; ¹H NMR (400 MHz, DMSO-d₆): δ 8.31-8.24 (m, 1H), 8.18 (br d, J=5.5 Hz, 1H), 7.81-7.58 (m, 1H), 7.33-7.05 (m, 3H), 6.69-6.62 (m, 1H), 4.80-4.69 (m, 1H), 4.40-4.07 (m, 4H), 3.99-3.66 (m, 2H), 3.36 (s, 2H), 3.01-2.91 (m, 1H), 2.86-2.72 (m, 5H), 2.65-2.59 (m, 2H), 2.39-2.31 (m, 6H), 2.29-2.21 (m, 2H), 2.06-1.85 (m, 4H), 1.78-1.68 (m, 11H), 1.60-1.50 (m, 2H), 1.47-0.84 (m, 6H), one proton merged with solvent peaks; LCMS (Method A): Rt=1.62 min, m/z=656.2 [M+H]⁺; HPLC (Method C): Rt=8.34 min, 98.15%; Chiral SFC (Method AAI): Rt=1.50 min, 100%.

### Example 175. 5-Fluoro-N-isopropyl-N-((1r,3r)-3-methyl-3-(pyrrolidin-1-yl)cyclobutyl)-2-((5-(6-((6-methyl-5,6,7,8-tetrahydro-1,6-naphthyridin-4-yl)oxy)-2-azaspiro[3.3]heptan-2-yl)-1,2,4-triazin-6-yl)oxy)benzamide (Compound No. 175)

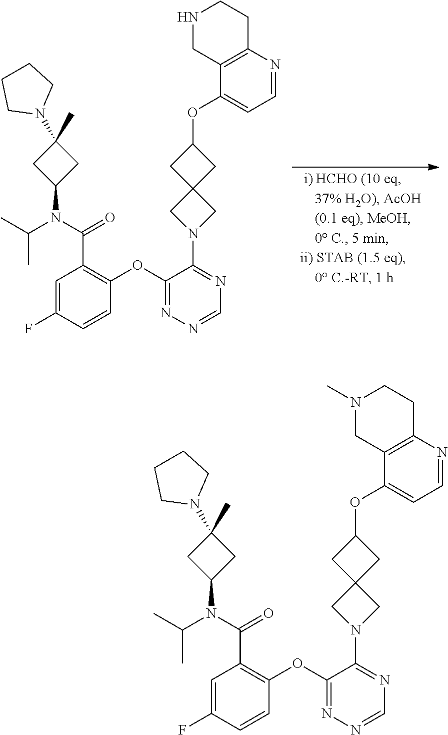 [SMILES: CC(C)N(C(=O)c1cc(F)ccc1Oc1nncnc1N1CC2(CC(Oc3ccnc4c3CNCC4)C2)C1)[C@H]1C[C@](C)(N2CCCC2)C1 ; CC(C)N(C(=O)c1cc(F)ccc1Oc1nncnc1N1CC2(CC(Oc3ccnc4c3CN(C)CC4)C2)C1)[C@H]1C[C@](C)(N2CCCC2)C1]

This compound was synthesized following the procedure described for the synthesis of Example 76, starting with 1 equivalent of 5-fluoro-N-isopropyl-N-((1r,3r)-3-methyl-3-(pyrrolidin-1-yl)cyclobutyl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide, except that 10 equivalents of formaldehyde (37% in H₂O), 0.1 equivalents of AcOH, and 1.5 equivalents of STAB were used. After completion, the reaction was quenched with aqueous NaHCO₃ solution, and the mixture was extracted with 10% MeOH in DCM (10% MeOH in DCM). The combined organic layer was dried over anhydrous sodium sulfate and filtered, and the filtrate was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN), and then by Chiral Prep SFC (Method T) to obtain 5-fluoro-N-isopropyl-N-((1r,3r)-3-methyl-3-(pyrrolidin-1-yl)cyclobutyl)-2-((5-(6-((6-methyl-5,6,7,8-tetrahydro-1,6-naphthyridin-4-yl)oxy)-2-azaspiro[3.3]heptan-2-yl)-1,2,4-triazin-6-yl)oxy)benzamide as a solid.

Yield: 22.90%; ¹H NMR (400 MHz, DMSO-d₆): δ8.46 (s, 1H), 8.19 (br d, J=5.0 Hz, 1H), 7.54-7.11 (m, 3H), 6.76-6.60 (m, 1H), 4.85-4.75 (m, 1H), 4.74-4.47 (m, 2H), 4.34-4.09 (m, 2H), 4.04-3.69 (m, 1H), 3.67-3.53 (m, 1H), 3.38 (s, 2H), 2.82 (br t, J=5.5 Hz, 4H), 2.66-2.60 (m, 2H), 2.36 (m, 3H), 2.32-2.25 (m, 3H), 2.24-2.08 (m, 2H), 1.87-1.74 (m, 1H), 1.70-1.51 (m, 5H), 1.46-0.46 (m, 9H), three protons merged with solvent peaks; LCMS (Method H): Rt=1.28 min, m/z=671.4 [M+H]⁺; HPLC (Method G): Rt=6.43 min, 98.78%; Chiral SFC (Method C): Rt=3.45 min, 100%.

### Example 176. (D2)-N-((1r,3r)-3-(Azetidin-1-yl)-3-methylcyclobutyl)-2-((4-(6-((5-ethyl-6-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (Compound No. 176)

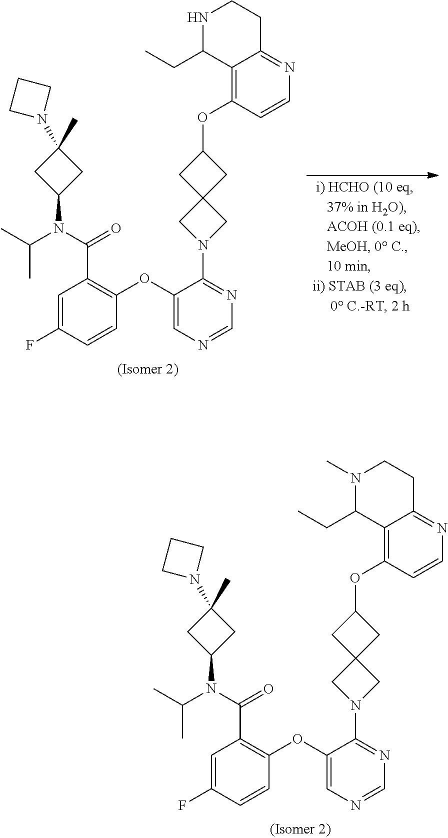 [SMILES: CCC1NCCc2nccc(OC3CC4(C3)CN(c3ncncc3Oc3ccc(F)cc3C(=O)N(C(C)C)[C@H]3C[C@](C)(N5CCC5)C3)C4)c21 ; CCC1c2c(OC3CC4(C3)CN(c3ncncc3Oc3ccc(F)cc3C(=O)N(C(C)C)[C@H]3C[C@](C)(N5CCC5)C3)C4)ccnc2CCN1C]

The reaction was performed following the same procedure in two batches.

This compound was synthesized following the procedure described for the synthesis of Example 76, starting with 1 equivalent of (D2)-N-((1r,3r)-3-(azetidin-1-yl)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide, except that 10 equivalents of formaldehyde (37% in H₂O), 0.1 equivalent of AcOH were used. After completion, the reaction was concentrated under reduced pressure. The residue was diluted with aqueous 10% NaHCO₃ solution and extracted with 10% MeOH in DCM. The combined organic layer was dried over anhydrous sodium sulfate and filtered, and the filtrate was concentrated under reduced pressure. After workup, the crude from both batches were combined and purified by reverse phase chromatography using a SiliCycle SiliaSep C18 column (12 g) on Biotage isolera one (Mobile phase A: 10 mM NH₄HCO₃ in water, B: ACN) to obtain (D2)-N-((1r,3r)-3-(azetidin-1-yl)-3-methylcyclobutyl)-2-((4-(6-((5-ethyl-6-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide as a solid.

Yield: 40.32%; ¹H NMR (400 MHz, DMSO-d₆): δ 8.31-8.24 (m, 1H), 8.17 (d, J=5.6 Hz, 1H), 7.82-7.56 (m, 1H), 7.37-7.05 (m, 3H), 6.69-6.62 (m, 1H), 4.80-4.69 (m, 1H), 4.40-4.12 (m, 4H), 3.81-3.64 (m, 2H), 3.49-3.42 (m, 1H), 3.17-3.07 (m, 3H), 2.96-2.66 (m, 7H), 2.62-2.55 (m, 1H), 2.32 (s, 3H), 2.29-1.97 (m, 4H), 1.74-1.74 (m, 1H), 1.92-1.73 (m, 1H), 1.65-1.49 (m, 2H), 1.47-0.81 (m, 12H), one proton merged with solvent peaks; LCMS (Method H): Rt=1.05 min, m/z=684.3 [M+H]⁺; HPLC (Method G): Rt=6.36 min, 99.00%; Chiral SFC (Method Y): Rt=4.60 min, 99.27%.

### Example 177. (D2)-2-((4-(6-((5-Ethyl-6-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide (Compound No. 177)

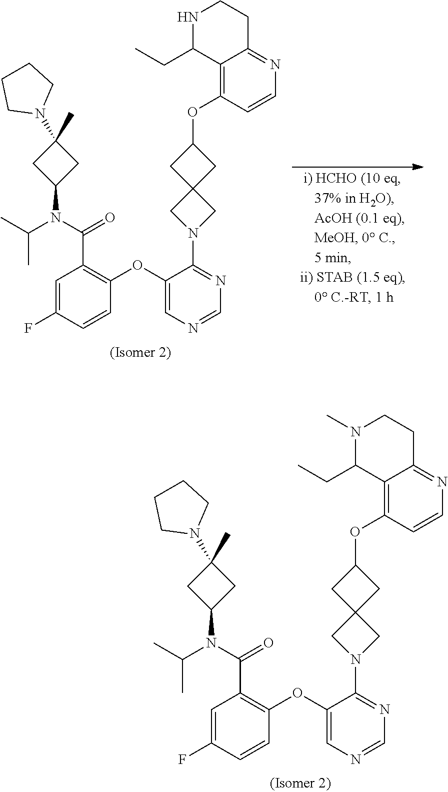 [SMILES: CCC1NCCc2nccc(OC3CC4(C3)CN(c3ncncc3Oc3ccc(F)cc3C(=O)N(C(C)C)[C@H]3C[C@](C)(N5CCCC5)C3)C4)c21 ; CCC1c2c(OC3CC4(C3)CN(c3ncncc3Oc3ccc(F)cc3C(=O)N(C(C)C)[C@H]3C[C@](C)(N5CCCC5)C3)C4)ccnc2CCN1C]

This compound was synthesized following the procedure described for the synthesis of Example 76, starting with 1 equivalent of (D2)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide, except that 10 equivalents of formaldehyde (37% in H₂O), 0.1 equivalent of AcOH, and 1.5 equivalents of STAB were used. After work-up, the crude compound was purified by reverse phase chromatography using a SiliCycle SiliaSep C18 column (12 g) on Biotage isolera one (Mobile phase A: 10 mM NH₄HCO₃ in water, B: ACN) to afford (D2)-2-((4-(6-((5-ethyl-6-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide as a solid.

Yield: 84%; ¹H NMR (400 MHz, DMSO-d₆): δ 8.33-8.25 (m, 1H), 8.17 (d, J=5.4 Hz, 1H), 7.80-7.55 (m, 1H), 7.34-7.04 (m, 3H), 6.70-6.62 (m, 1H), 4.74 (quin, J=6.5 Hz, 1H), 4.41-3.79 (m, 5H), 3.76-3.63 (m, 1H), 3.51-3.43 (m, 1H), 3.17-3.06 (m, 2H), 2.90-2.71 (m, 4H), 2.41-2.36 (m, 2H), 2.35-2.31 (m, 4H), 2.29-2.05 (m, 5H), 2.00-1.73 (m, 2H), 1.71-1.52 (m, 6H), 1.46-0.91 (m, 9H), 0.88 (br t, J=7.1 Hz, 3H); LCMS (Method H): Rt=1.10 min, m/z=698.3 [M+H]⁺; HPLC (Method G): Rt=6.59 min, 99.50%; Chiral SFC (Method AAF): Rt=5.75 min, 100%.

### Example 178. (D2)-2-((4-(6-((5-Ethyl-6-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide (Compound No. 178)

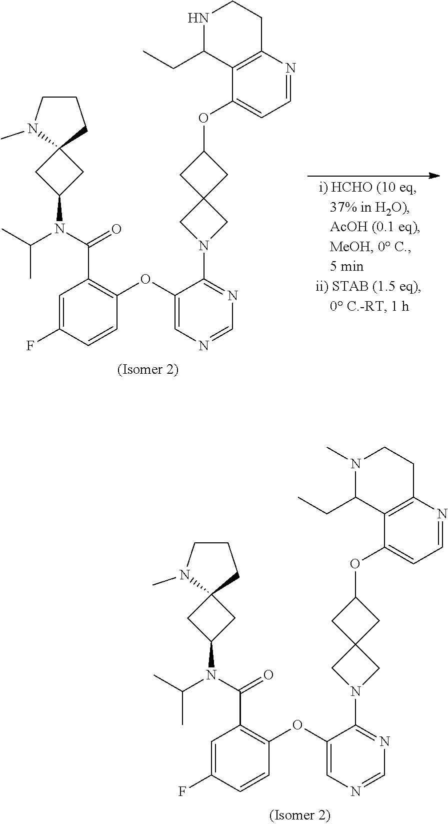 [SMILES: CCC1NCCc2nccc(OC3CC4(C3)CN(c3ncncc3Oc3ccc(F)cc3C(=O)N(C(C)C)[C@H]3C[C@@]5(CCCN5C)C3)C4)c21 ; CCC1c2c(OC3CC4(C3)CN(c3ncncc3Oc3ccc(F)cc3C(=O)N(C(C)C)[C@H]3C[C@@]5(CCCN5C)C3)C4)ccnc2CCN1C]

This compound was synthesized following the procedure described for the synthesis of Example 76, starting with 1 equivalent of (D2)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide, except that 10 equivalents of formaldehyde (37% in H₂O), 0.1 equivalent of AcOH, and 1.5 equivalents of STAB were used. After completion, the reaction was diluted with ice-cold water, aqueous Na₂CO₃ solution was added, and the mixture was extracted with EtOAc. The combined organic layer was washed with brine solution, dried over anhydrous sodium sulfate and filtered, and the filtrate was concentrated under reduced pressure. The crude compound was purified by reverse phase chromatography using a SiliCycle SiliaSep C18 column (30 g) on Biotage isolera one (Mobile phase A: 10 mM NH₄HCO₃ in water, B: ACN) to obtain (D2)-2-((4-(6-((5-ethyl-6-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide as a solid.

Yield: 70.8%; ¹H NMR (400 MHz, DMSO-d₆): δ 8.33-8.23 (m, 1H), 8.17 (br d, J=5.5 Hz, 1H), 7.80-7.56 (m, 1H), 7.34-7.06 (m, 3H), 6.69-6.63 (m, 1H), 4.80-4.69 (m, 1H), 4.43-4.10 (m, 4H), 3.97-3.62 (m, 2H), 3.46 (br d, J=6.0 Hz, 1H), 3.18-3.06 (m, 1H), 2.91-2.67 (m, 5H), 2.46-2.40 (m, 1H), 2.36-2.30 (m, 6H), 2.29-2.17 (m, 3H), 2.07-1.86 (m, 4H), 1.76-1.48 (m, 5H), 1.47-0.84 (m, 9H), one proton merged with solvent peaks; LCMS (Method H): Rt=1.10 min, m/z=684.4 [M+H]⁺; HPLC (Method G): Rt=6.35 min, 99.72%; Chiral SFC (Method J): Rt=4.35 min, 99.83%.

### Example 179. (D2)-2-((4-(6-((5,6-Dimethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide (Compound No. 179)

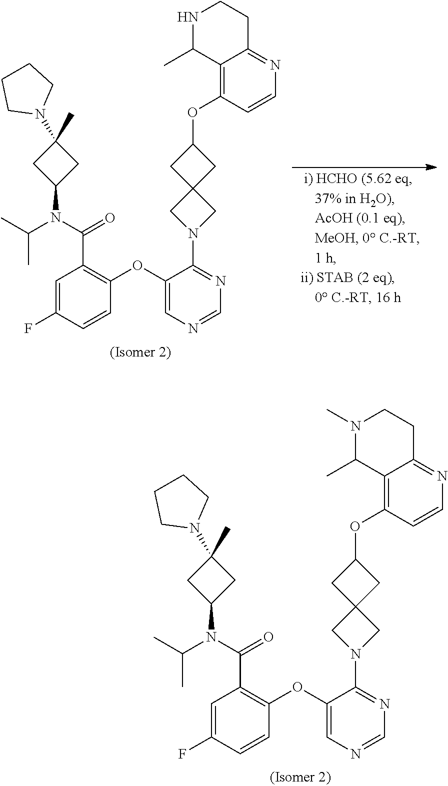 [SMILES: CC1NCCc2nccc(OC3CC4(C3)CN(c3ncncc3Oc3ccc(F)cc3C(=O)N(C(C)C)[C@H]3C[C@](C)(N5CCCC5)C3)C4)c21 ; CC1c2c(OC3CC4(C3)CN(c3ncncc3Oc3ccc(F)cc3C(=O)N(C(C)C)[C@H]3C[C@](C)(N5CCCC5)C3)C4)ccnc2CCN1C]

This compound was synthesized following the procedure described for the synthesis of Example 76, starting with 1 equivalent of (D2)-5-fluoro-N-isopropyl-N-((1r,3r)-3-methyl-3-(pyrrolidin-1-yl)cyclobutyl)-2-((4-(6-(((R)-5-methyl-5,6,7,8-tetrahydro-1,6-naphthyridin-4-yl)oxy)-2-azaspiro[3.3]heptan-2-yl)pyrimidin-5-yl)oxy)benzamide, except that 5.62 equivalents of formaldehyde (37% in H₂O), 0.1 equivalent of AcOH, and 2 equivalents of STAB were used. After completion, the reaction was concentrated under reduced pressure. The residue was dissolved in sodium bicarbonate solution and the mixture was extracted with 10% MeOH in DCM. The combined organic layer was dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford (D2)-2-((4-(6-((5,6-dimethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((1r,3r)-3-methyl-3-(pyrrolidin-1-yl)cyclobutyl)benzamide as a solid.

Yield: 49.9%; ¹H NMR (400 MHz, DMSO-d₆): δ 8.31-8.23 (m, 1H), 8.17 (d, J=5.6 Hz, 1H), 7.78-7.58 (m, 1H), 7.31-7.07 (m, 3H), 6.69-6.60 (m, 1H), 4.75 (quin, J=6.6 Hz, 1H), 4.34-4.01 (m, 5H), 3.82 (q, J=6.3 Hz, 1H), 3.76-3.64 (m, 1H), 3.03-2.93 (m, 1H), 2.89-2.75 (m, 4H), 2.70-2.64 (m, 2H), 2.40-2.31 (m, 5H), 2.30-1.90 (m, 4H), 1.85-1.75 (m, 1H), 1.71-1.54 (m, 4H), 1.47-0.89 (m, 12H), two protons merged with solvent peaks; LCMS (Method H): Rt=1.28 min, m/z=684.4 [M+H]⁺; HPLC (Method C): Rt=8.62 min, 98.45%; Chiral SFC (Method Y): Rt=5.33 min, 95.76%.

### Example 180. (D2)-N-((1r,3r)-3-(Azetidin-1-yl)-3-methylcyclobutyl)-2-((4-(6-((5,6-dimethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (Compound No. 180)

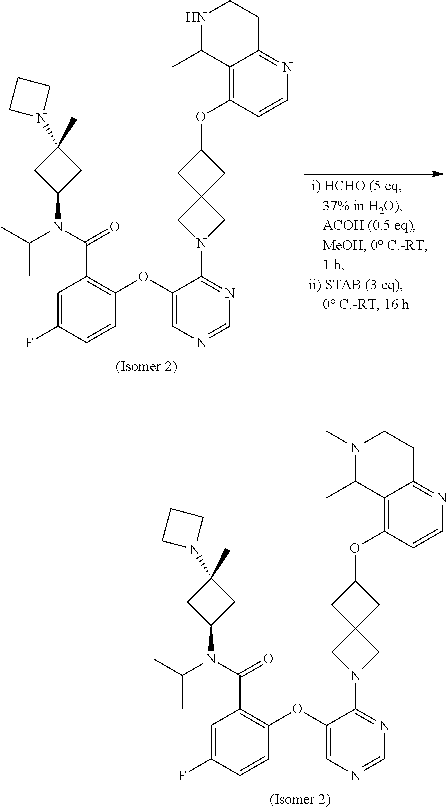 [SMILES: CC1NCCc2nccc(OC3CC4(C3)CN(c3ncncc3Oc3ccc(F)cc3C(=O)N(C(C)C)[C@H]3C[C@](C)(N5CCC5)C3)C4)c21 ; CC1c2c(OC3CC4(C3)CN(c3ncncc3Oc3ccc(F)cc3C(=O)N(C(C)C)[C@H]3C[C@](C)(N5CCC5)C3)C4)ccnc2CCN1C]

This compound was synthesized following the procedure described for the synthesis of Example 76, starting with 1 equivalent of ((D2)-N-((1r,3r)-3-(azetidin-1-yl)-3-methylcyclobutyl)-2-((4-(6-((5,6-dimethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide, except that 5 equivalents of formaldehyde (37% in H₂O) and 0.5 equivalent of AcOH were used. After completion, the reaction was concentrated under reduced pressure. The residue was diluted with saturated sodium bicarbonate solution and the mixture was extracted with 10% MeOH in DCM. The combined organic layer was dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford (D2)-N-((1r,3r)-3-(azetidin-1-yl)-3-methylcyclobutyl)-2-((4-(6-((5,6-dimethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide as a solid.

Yield: 38.39%; ¹H NMR (400 MHz, DMSO-d₆): δ 8.30-8.24 (m, 1H), 8.17 (d, J=5.6 Hz, 1H), 7.78-7.60 (m, 1H), 7.33-7.05 (m, 3H), 6.71-6.60 (m, 1H), 4.80-4.68 (m, 1H), 4.38-3.91 (m, 4H), 3.82 (q, J=6.6 Hz, 1H), 3.77-3.63 (m, 2H), 3.11 (br t, J=6.7 Hz, 2H), 3.03-2.72 (m, 6H), 2.67-2.59 (m, 2H), 2.34 (s, 3H), 2.30-1.97 (m, 5H), 1.92-1.71 (m, 3H), 1.48-0.89 (m, 12H); LCMS (Method H): Rt=1.13 min, m/z=670.4 [M+H]⁺; HPLC (Method G): Rt=6.29 min, 99.83%; Chiral SFC (Method J): Rt=5.39 min, 98.07%.

### Example 181. (D2)-N-((1r,3r)-3-(Dimethylamino)-3-methylcyclobutyl)-2-((4-(6-((5-ethyl-6-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide (Compound No. 181)

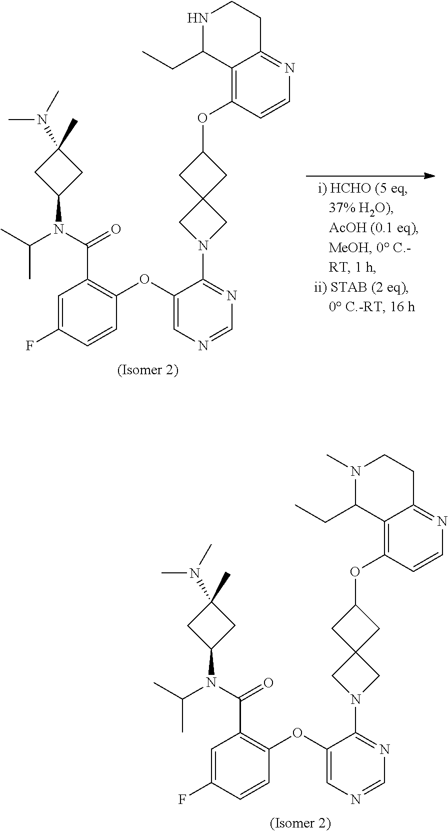 [SMILES: CCC1NCCc2nccc(OC3CC4(C3)CN(c3ncncc3Oc3ccc(F)cc3C(=O)N(C(C)C)[C@H]3C[C@](C)(N(C)C)C3)C4)c21 ; CCC1c2c(OC3CC4(C3)CN(c3ncncc3Oc3ccc(F)cc3C(=O)N(C(C)C)[C@H]3C[C@](C)(N(C)C)C3)C4)ccnc2CCN1C]

This compound was synthesized following the procedure described for the synthesis of Example 76, starting with 1 equivalent of (D2)-N-((1r,3r)-3-(dimethylamino)-3-methylcyclobutyl)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide, except that 5 equivalents of formaldehyde (37% in H₂O), 0.1 equivalent of AcOH, and 2 equivalents of STAB were used. After completion, the reaction was concentrated under reduced pressure. The crude was dissolved in 10% MeOH in DCM and the mixture was washed with aqueous sodium bicarbonate solution. The combined organic layer was dried over anhydrous sodium sulfate and filtered, and the filtrate was concentrated under reduced pressure. The crude was purified by reverse phase chromatography using a SiliCycle SiliaSep C18 column (12 g) on Biotage isolera one (Mobile phase A: 10 mM NH₄HCO₃ in water, B: ACN) to obtain (D2)-N-((1r,3r)-3-(Dimethylamino)-3-methylcyclobutyl)-2-((4-(6-((5-ethyl-6-methyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamide as a solid.

Yield: 43.4%; ¹H NMR (400 MHz, DMSO-d₆): δ 8.31-8.22 (m, 1H), 8.19-8.14 (m, 1H), 7.79-7.57 (m, 1H), 7.32-7.04 (m, 3H), 6.71-6.60 (m, 1H), 4.74 (br t, J=6.4 Hz, 1H), 4.34-4.09 (m, 4H), 4.04-3.77 (m, 1H), 3.75-3.65 (m, 1H), 3.46 (br dd, J=4.1, 7.0 Hz, 1H), 3.16-3.07 (m, 1H), 2.89-2.71 (m, 4H), 2.33 (s, 3H), 2.29-2.17 (m, 3H), 2.14-1.87 (m, 8H), 1.81-1.68 (m, 1H), 1.63-1.51 (m, 2H), 1.46-1.23 (m, 3H), 1.16-0.84 (m, 10H); LCMS (Method A): Rt=2.00 min, m/z=672.5 [M+H]⁺; HPLC (Method C): Rt=9.10 min, 98.05%; Chiral SFC (Method AV): Rt=2.67 min, 100%.

### Example 182. N-((1r,3r)-3-(Dimethylamino)-3-methylcyclobutyl)-5-fluoro-N-isopropyl-2-((5-(6-((2-methyl-1,2,3,4-tetrahydroisoquinolin-8-yl)oxy)-2-azaspiro[3.3]heptan-2-yl)-1,2,4-triazin-6-yl)oxy)benzamide (Compound No. 182)

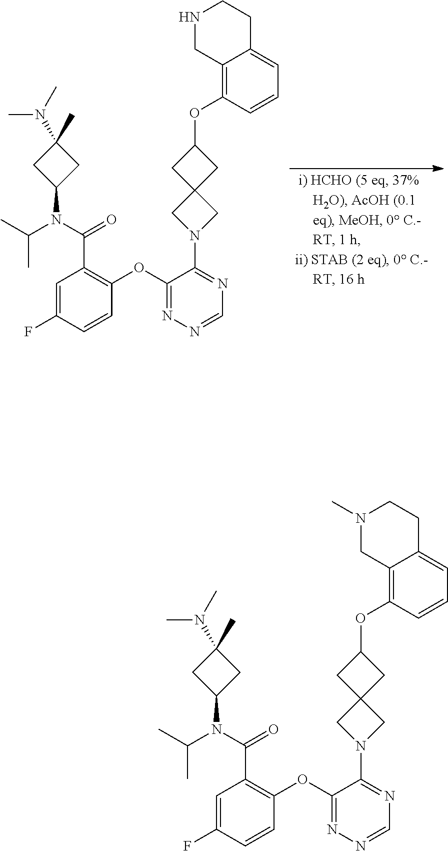 [SMILES: CC(C)N(C(=O)c1cc(F)ccc1Oc1nncnc1N1CC2(CC(Oc3cccc4c3CNCC4)C2)C1)[C@H]1C[C@](C)(N(C)C)C1 ; CC(C)N(C(=O)c1cc(F)ccc1Oc1nncnc1N1CC2(CC(Oc3cccc4c3CN(C)CC4)C2)C1)[C@H]1C[C@](C)(N(C)C)C1]

This compound was synthesized following the procedure described for the synthesis of Example 76, starting with 1 equivalent of N-((1r,3r)-3-(dimethylamino)-3-methylcyclobutyl)-5-fluoro-N-isopropyl-2-((5-(6-((1,2,3,4-tetrahydroisoquinolin-8-yl)oxy)-2-azaspiro[3.3]heptan-2-yl)-1,2,4-triazin-6-yl)oxy)benzamide, except that 5 equivalents of formaldehyde (37% in H₂O), 0.1 equivalent of AcOH, and 2 equivalents of STAB were used. After completion, the reaction was concentrated under reduced pressure. The residue was dissolved in sodium bicarbonate solution and the mixture was extracted with 10% MeOH in DCM. The combined organic layer was dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to afford N-((1r,3r)-3-(dimethylamino)-3-methylcyclobutyl)-5-fluoro-N-isopropyl-2-((5-(6-((2-methyl-1,2,3,4-tetrahydroisoquinolin-8-yl)oxy)-2-azaspiro[3.3]heptan-2-yl)-1,2,4-triazin-6-yl)oxy)benzamide as a solid.

Yield: 56.1%; ¹H NMR (400 MHz, DMSO-d₆): δ 8.46 (s, 1H), 7.50-7.13 (m, 3H), 7.11-6.96 (m, 1H), 6.69 (d, J=7.6 Hz, 1H), 6.65-6.50 (m, 1H), 4.75-4.48 (m, 3H), 4.30-4.09 (m, 2H), 3.99-3.69 (m, 1H), 3.66-3.49 (m, 1H), 3.35 (br s, 2H), 2.86-2.73 (m, 4H), 2.34 (br s, 3H), 2.31-2.22 (m, 2H), 2.15-1.92 (m, 8H), 1.85-1.48 (m, 2H), 1.45-0.58 (m, 9H), two protons merged with solvent peaks; LCMS (Method A): Rt=2.02 min, m/z=644.2 [M+H]⁺, 95.05%; HPLC (Method G): Rt=7.11 min, 97.96%; Chiral SFC (Method J): Rt=5.64 min, 98.66%.

### Example 183. N-((1r,3r)-3-(Ethyl(methyl)amino)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 183)

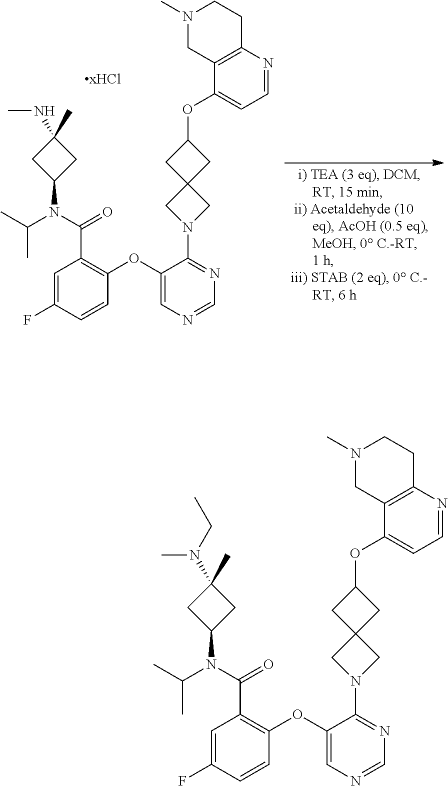 [SMILES: CN[C@]1(C)C[C@@H](N(C(=O)c2cc(F)ccc2Oc2cncnc2N2CC3(CC(Oc4ccnc5c4CN(C)CC5)C3)C2)C(C)C)C1 ; CCN(C)[C@]1(C)C[C@@H](N(C(=O)c2cc(F)ccc2Oc2cncnc2N2CC3(CC(Oc4ccnc5c4CN(C)CC5)C3)C2)C(C)C)C1]

In a 10 mL round bottom flask under a nitrogen atmosphere, 5-fluoro-N-isopropyl-N-((1r,3r)-3-methyl-3-(methylamino)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl (200 mg, 0.300 mmol) was dissolved in DCM (5 mL). To this solution, TEA (0.126 mL, 0.901 mmol) was added at 0° C. The reaction was stirred at RT for 15 min and then concentrated under reduced pressure. The residue was dissolved in MeOH (5 mL), acetaldehyde (0.168 mL, 3.00 mmol), and acetic acid (8.58 μL, 0.150 mmol) were added at 0° C., and the reaction was stirred at RT for 1 h. To this reaction mixture, sodium triacetoxyborohydride (127 mg, 0.600 mmol) was added at 0° C. The reaction was stirred at RT for 6 h. The reaction progress was monitored by TLC (10% MeOH in DCM). After completion, the reaction was concentrated under reduced pressure, the crude was diluted with saturated sodium bicarbonate solution (10 mL), and the mixture was extracted with DCM (3×10 mL). The combined organic layer was dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to obtain N-((1r,3r)-3-(ethyl(methyl)amino)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (62 mg, 30.7% yield) as a solid. ¹H NMR (400 MHz, DMSO-d₆): δ 8.30-8.23 (m, 1H), 8.18 (br d, J=5.1 Hz, 1H), 7.81-7.56 (m, 1H), 7.32-7.05 (m, 3H), 6.70-6.60 (m, 1H), 4.75 (quin, J=6.5 Hz, 1H), 4.36-4.09 (m, 4H), 4.07-3.62 (m, 2H), 3.36 (br s, 2H), 2.85-2.74 (m, 4H), 2.63 (br t, J=5.7 Hz, 2H), 2.36 (s, 3H), 2.31-2.14 (m, 4H), 2.11-1.97 (m, 4H), 1.93-1.67 (m, 2H), 1.47-1.27 (m, 3H), 1.15-0.82 (m, 10H); LCMS (Method A): Rt=1.83 min, m/z=658.2 [M+H]⁺; HPLC (Method A): Rt=5.09 min, 97.79%; Chiral SFC (Method AM): Rt=4.87 min, 100%.

### Example 184. 5-Fluoro-N-isopropyl-N-((1r,3r)-3-((2-methoxyethyl)(methyl)amino)-3-methylcyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 184)

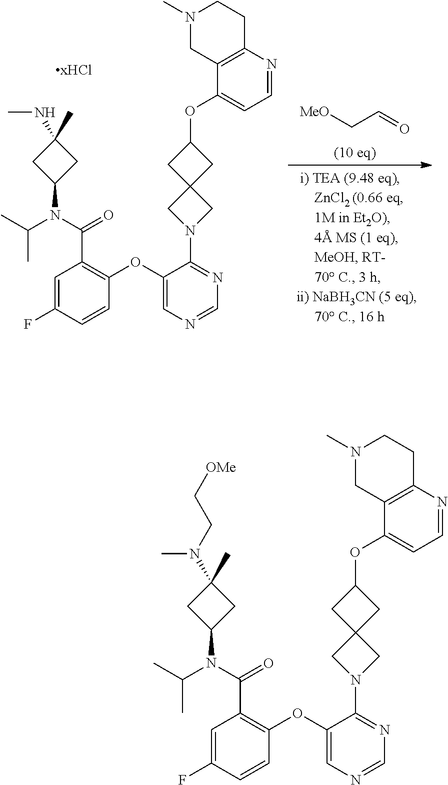 [SMILES: CN[C@]1(C)C[C@@H](N(C(=O)c2cc(F)ccc2Oc2cncnc2N2CC3(CC(Oc4ccnc5c4CN(C)CC5)C3)C2)C(C)C)C1 ; COCC=O ; COCCN(C)[C@]1(C)C[C@@H](N(C(=O)c2cc(F)ccc2Oc2cncnc2N2CC3(CC(Oc4ccnc5c4CN(C)CC5)C3)C2)C(C)C)C1]

In a 4 mL pressure vial, 5-fluoro-N-isopropyl-N-((1r,3r)-3-methyl-3-(methylamino)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl (0.09 g, 0.135 mmol) was dissolved in MeOH (2 mL). To this solution, triethylamine (0.130 g, 1.281 mmol) was added at RT. To this reaction mixture, 2-methoxyacetaldehyde (0.100 g, 1.351 mmol), 4 Å molecular sieves (0.027 g, 0.135 mmol), and zinc chloride (0.089 mL, 0.089 mmol, 1 M in Et₂O) were added at RT. The reaction was stirred at 70° C. for 3 h. The reaction was cooled to RT and sodium cyanoborohydride (0.042 g, 0.675 mmol) was added. The reaction was stirred at 70° C. for 16 h. The reaction progress was monitored by TLC (10% MeOH in DCM) and LCMS. After completion, the reaction was diluted with water (10 mL) and the mixture was extracted with 10% MeOH-DCM (2×20 mL). The combined organic layer was washed with saturated aqueous sodium bicarbonate solution (10 mL). The organic layer was dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN), then by Prep HPLC (Method E) to obtain 5-fluoro-N-isopropyl-N-((1r,3r)-3-((2-methoxyethyl)(methyl)amino)-3-methylcyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (15 mg, 15.77% yield) as a solid. ¹H NMR (400 MHz, DMSO-d₆): δ 8.31-8.23 (m, 1H), 8.18 (d, J=5.5 Hz, 1H), 7.78-7.63 (m, 1H), 7.31-7.05 (m, 3H), 6.70-6.59 (m, 1H), 4.75 (quin, J=6.6 Hz, 1H), 4.35-4.09 (m, 4H), 4.07-3.64 (m, 2H), 3.36 (br s, 2H), 3.26-3.10 (m, 5H), 2.85-2.74 (m, 4H), 2.63 (br t, J=5.8 Hz, 2H), 2.45-2.40 (m, 2H), 2.37 (s, 3H), 2.31-2.22 (m, 3H), 2.17-2.01 (m, 4H), 1.97-1.69 (m, 2H), 1.46-1.27 (m, 3H), 1.16-0.90 (m, 6H); LCMS (Method C): Rt=1.43 min, m/z=688.4 [M+H]⁺; HPLC (Method E): Rt=9.58 min, 97.68%; Chiral SFC (Method C): Rt=2.57 min, 100%.

### Example 185. 5-Fluoro-N-isopropyl-N-((1r,3r)-3-methyl-3-(methyl(oxetan-3-yl)amino)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 185)

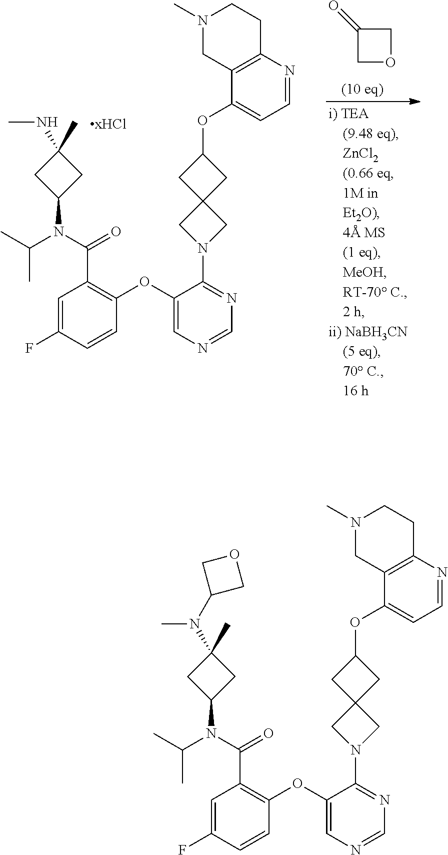 [SMILES: CN[C@]1(C)C[C@@H](N(C(=O)c2cc(F)ccc2Oc2cncnc2N2CC3(CC(Oc4ccnc5c4CN(C)CC5)C3)C2)C(C)C)C1 ; O=C1COC1 ; CC(C)N(C(=O)c1cc(F)ccc1Oc1cncnc1N1CC2(CC(Oc3ccnc4c3CN(C)CC4)C2)C1)[C@H]1C[C@](C)(N(C)C2COC2)C1]

This compound was synthesized following the procedure described for the synthesis of Example 184, starting with 1 equivalent of 5-fluoro-N-isopropyl-N-((1r,3r)-3-methyl-3-(methylamino)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide xHCl and 10 equivalents of oxetan-3-one. After completion, the reaction was diluted with saturated aqueous sodium bicarbonate solution and the mixture was extracted with DCM. The combined organic layer was dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford 5-fluoro-N-isopropyl-N-((1r,3r)-3-methyl-3-(methyl(oxetan-3-yl)amino)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 5.81%; ¹H NMR (400 MHz, DMSO-d₆): δ 8.28 (s, 1H), 8.18 (d, J=5.5 Hz, 1H), 7.77-7.56 (m, 1H), 7.31-7.04 (m, 3H), 6.69-6.61 (m, 1H), 4.75 (quin, J=6.5 Hz, 1H), 4.67 (br t, J=6.6 Hz, 1H), 4.56-4.35 (m, 3H), 4.34-4.09 (m, 4H), 4.04-3.88 (m, 1H), 3.83-3.63 (m, 2H), 3.36 (br s, 2H), 2.85-2.76 (m, 4H), 2.63 (br t, J=5.8 Hz, 2H), 2.36 (s, 3H), 2.32-2.16 (m, 5H), 2.16-1.97 (m, 2H), 1.93-1.67 (m, 2H), 1.46-1.23 (m, 3H), 1.09-0.84 (m, 6H); LCMS (Method A): Rt=1.70 min, m/z=686.2 [M+H]⁺; HPLC (Method A): Rt=4.93 min, 99.65%; Chiral SFC (Method AQ): Rt=2.2 min, 100%.

### Example 186. N-((1r,3r)-3-((2,2-Difluoroethyl)(methyl)amino)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 186)

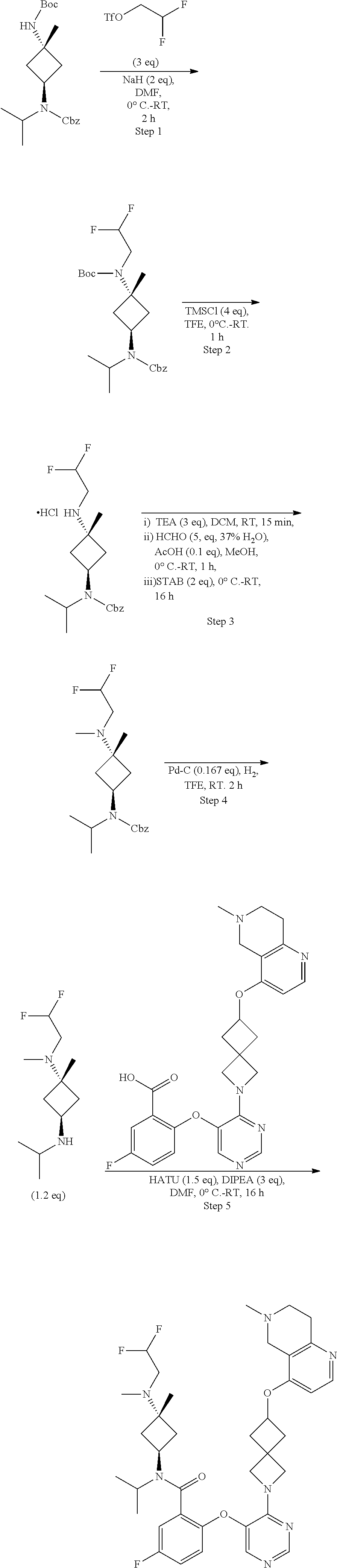

### Step 1. Benzyl ((1r,3r)-3-((tert-butoxycarbonyl)(2,2-difluoroethyl)amino)-3-methylcyclobutyl)(isopropyl)carbamate

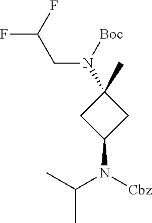 [SMILES: CC(C)N(C(=O)OCc1ccccc1)[C@H]1C[C@](C)(N(CC(F)F)C(=O)OC(C)(C)C)C1]

In a dried, 25 mL two neck round bottom flask under a nitrogen atmosphere, benzyl ((1r,3r)-3-((tert-butoxycarbonyl)amino)-3-methylcyclobutyl)(isopropyl)carbamate (0.43 g, 1.142 mmol) was dissolved in DMF (5 mL). To this solution, sodium hydride (0.091 g, 2.284 mmol) was added at 0° C., and the reaction was stirred at RT for 15 min. To this reaction mixture, 2,2-difluoroethyl trifluoromethanesulfonate (0.734 g, 3.43 mmol) was added at 0° C. The reaction was stirred at RT for 2 h. The reaction was monitored by LCMS and TLC (20% EtOAc in hexane). After completion, the reaction was quenched with saturated ammonium chloride solution (20 mL) and the mixture was extracted with EtOAc (2×20 mL). The combined organic layer was washed with brine (10 mL), dried over anhydrous sodium sulfate and filtered, and the filtrate was concentrated under reduced pressure. The crude product was purified by silica gel flash column chromatography using EtOAc in hexane (product eluted at 10% EtOAc in hexane) to obtain benzyl ((1r,3r)-3-((tert-butoxycarbonyl)(2,2-difluoroethyl)amino)-3-methylcyclobutyl)(isopropyl)carbamate (0.48 g, 95% yield) as a liquid. ¹H NMR (400 MHz, DMSO-d₆): δ 7.43-7.30 (m, 5H), 6.28-5.92 (m, 1H), 5.08 (s, 2H), 4.03 (quin, J=6.8 Hz, 1H), 3.91 (quin, J=9.2 Hz, 1H), 3.53 (dt, 0.1=4.0, 14.4 Hz, 2H), 2.59-2.54 (m, 2H), 2.44-2.35 (m, 2H), 1.40 (s, 9H), 1.24 (br s, 3H), 1.10 (d, J=6.8 Hz, 6H); LCMS-ELSD (Method A): Rt=2.66 min, m/z=441.1 [M+H]⁺, 99.63%.

### Step 2. Benzyl ((1r,3r)-3-((2,2-difluoroethyl)amino)-3-methylcyclobutyl)(isopropyl)carbamate HCl

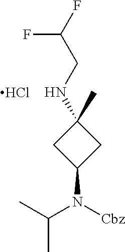 [SMILES: CC(C)N(C(=O)OCc1ccccc1)[C@H]1C[C@](C)(NCC(F)F)C1 ; Cl]

This compound was synthesized following the procedure described for the synthesis of Example 5, step 2, starting with 1 equivalent of benzyl ((1r,3r)-3-((tert-butoxycarbonyl)(2,2-difluoroethyl)amino)-3-methylcyclobutyl)(isopropyl)carbamate. After completion, the solvent was evaporated under reduced pressure, and the residue was triturated with EtOAc. The solvent was decanted, and the solid was dried under vacuum to obtain crude benzyl ((1r,3r)-3-((2,2-difluoroethyl)amino)-3-methylcyclobutyl)(isopropyl)carbamate hydrochloride as a solid. This material was used without further purification.

Quantitative yield; ¹H NMR (400 MHz, DMSO-d₆): δ 9.93 (br s, 2H), 7.45-7.29 (m, 5H), 6.75-6.35 (m, 1H), 5.11 (s, 2H), 4.64-4.51 (m, 1H), 4.29-4.17 (m, 1H), 3.37 (br s, 2H), 2.75-2.58 (m, 2H), 2.48-2.31 (m, 2H), 1.47-1.15 (m, 3H), 1.07 (br d, J=6.6 Hz, 6H); LCMS (Method B): Rt=1.34 min, m/z=341.3 [M+H]⁺, 93.98%.

### Step 3. Benzyl ((1r,3r)-3-((2,2-difluoroethyl)(methyl)amino)-3-methylcyclobutyl)(isopropyl)carbamate

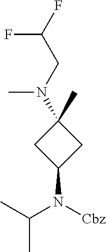 [SMILES: CC(C)N(C(=O)OCc1ccccc1)[C@H]1C[C@](C)(N(C)CC(F)F)C1]

This compound was synthesized following the procedure described for the synthesis of Example 20, step 4, starting with 1 equivalent of benzyl ((1r,3r)-3-((2,2-difluoroethyl)amino)-3-methylcyclobutyl)isopropyl)carbamate hydrochloride, except that 3 equivalents of TEA, 0.1 equivalent of AcOH, 5 equivalents of formaldehyde (37% in H₂O), and 2 equivalents of STAB were used. After completion, the reaction was quenched with aqueous sodium bicarbonate solution and the mixture was extracted with DCM. The combined organic layer was dried over anhydrous sodium sulfate and filtered, and the filtrate was concentrated under reduced pressure. The crude product was purified by silica gel flash column chromatography using EtOAc in hexane (product eluted at 10% EtOAc in hexane) to obtain benzyl ((1r,3r)-3-((2,2-difluoroethyl)(methyl)amino)-3-methylcyclobutyl)(isopropyl)carbamate as a liquid.

Yield: 50.5%; ¹H NMR (400 MHz, DMSO-d₆): δ 7.43-7.30 (m, 5H), 6.24-5.85 (m, 1H), 5.08 (s, 2H), 4.10-3.96 (m, 2H), 2.70-2.58 (m, 2H), 2.21 (s, 3H), 2.11 (br d, J=8.9 Hz, 4H), 1.11 (d, J=6.8 Hz, 6H), 0.95 (br s, 3H); LCMS (Method A): Rt=2.47 min, m/z=355.1 [M+H]⁺, 87.40%.

### Step 4. (1r,3r)—N¹-(2,2-Difluoroethyl)-N³-isopropyl-N¹,1-dimethylcyclobutane-1,3-diamine

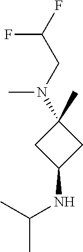

This compound was synthesized following the procedure described for the synthesis of Intermediate 20, step 5, starting with 1 equivalent of benzyl ((1r,3r)-3-((2,2-difluoroethyl)(methyl)amino)-3-methylcyclobutyl)(isopropyl)carbamate, except that 0.167 equivalent of Pd—C was used. After filtration, the filtrate was concentrated under reduced pressure to afford crude (1r,3r)—N¹-(2,2-difluoroethyl)-N-isopropyl-N¹,1-dimethylcyclobutane-1,3-diamine as a solid. This material was used without further purification.

Yield: 84%; ¹H NMR (400 MHz, DMSO-d₆): δ 6.17-5.83 (m, 1H), 3.22-3.12 (m, 11H), 2.73-2.55 (m, 4H), 2.22-2.14 (m, 5H), 1.44-1.36 (m, 2H), 1.04 (s, 3H), 0.92 (d, J=6.3 Hz, 6H).

### Step 5. N-((1r,3r)-3-((2,2-Difluoroethyl)(methyl)amino)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

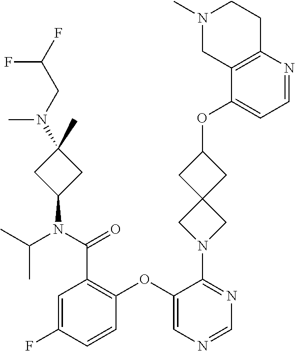 [SMILES: CC(C)N(C(=O)c1cc(F)ccc1Oc1cncnc1N1CC2(CC(Oc3ccnc4c3CN(C)CC4)C2)C1)[C@H]1C[C@](C)(N(C)CC(F)F)C1]

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.2 equivalents of (1r,3r)—N¹-(2,2-difluoroethyl)-N³-isopropyl-N¹,1-dimethylcyclobutane-1,3-diamine. After work-up, the crude compound was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford N-((1r,3r)-3-((2,2-difluoroethyl)(methyl)amino)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 36.5%; ¹H NMR (400 MHz, DMSO-d₆): δ 8.30-8.23 (m, 1H), 8.18 (d, J=5.5 Hz, 1H), 7.78-7.55 (m, 1H), 7.32-7.06 (m, 3H), 6.69-6.62 (m, 1H), 6.24-5.67 (m, 1H), 4.74 (quin, J=6.6 Hz, 1H), 4.35-4.07 (m, 4H), 4.06-3.65 (m, 2H), 3.36 (s, 2H), 2.86-2.74 (m, 4H), 2.71-2.59 (m, 4H), 2.36 (s, 3H), 2.30-2.10 (m, 6H), 2.09-1.70 (m, 3H), 1.47-1.23 (m, 3H), 1.18-0.91 (m, 6H); LCMS (Method A): Rt=2.01 min, m/z=694.2 [M+H]⁺; HPLC (Method A): Rt=6.05 min, 98.49%; Chiral SFC (Method AR): Rt=1.45 min, 100%.

### Example 187. 5-Fluoro-N-((1r,3r)-3-(3-fluoroazetidin-1-yl)cyclobutyl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 187)

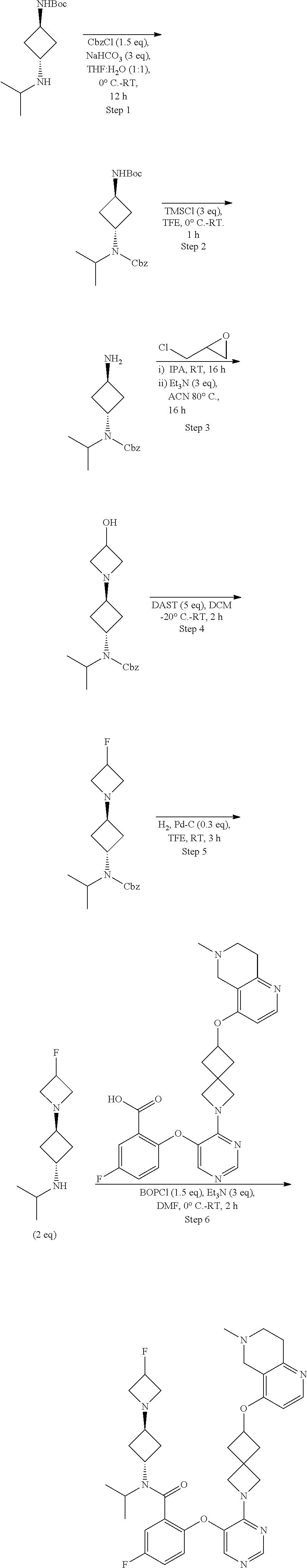

### Step 1. Benzyl ((1r,3r)-3-((tert-butoxycarbonyl)amino)cyclobutyl)(isopropyl)carbamate

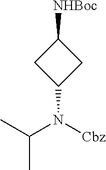

To a stirred solution of tert-butyl ((1r,3r)-3-(isopropyl amino)cyclobutyl)carbamate (5.2 g, 22.77 mmol) in THF:H₂O (1:1, 60 mL) at 0° C., sodium bicarbonate (5.74 g, 68.3 mmol), and Cbz-Cl (4.87 mL, 34.2 mmol) were added at 0° C. The reaction was stirred at RT for 12 h. The reaction progress was monitored by TLC (25% EtOAc in hexane). After completion, the reaction was quenched with ice cold water (50 mL), and the mixture was extracted with DCM (2×50 mL). The combined organic layer was washed with brine solution (50 mL), dried over anhydrous Na₂SO₄ and filtered, and the filtrate was concentrated under reduced pressure. The crude was purified by silica gel flash column chromatography using EtOAc in hexane (product was eluted at 18% EtOAc in hexane) to obtain benzyl ((1r,3r)-3-((tert-butoxycarbonyl)amino)cyclobutyl)(isopropyl)carbamate (4.1 g, 46.2% yield) as a solid. LCMS (Method A): Rt=2.22 min, m/z=361.2 [M−H]⁻, 93.24%.

### Step 2. Benzyl ((1r,3r)-3-aminocyclobutyl)(isopropyl)carbamate

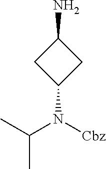 [SMILES: CC(C)N(C(=O)OCc1ccccc1)[C@H]1C[C@H](N)C1]

To a stirred solution of benzyl ((1r,3r)-3-((tert-butoxycarbonyl)amino)cyclobutyl)(isopropyl)carbamate (4.1 g, 11.31 mmol) in TFE (40 mL), TMSCl (4.31 mL, 33.9 mmol) was added at 0° C. and the reaction was stirred at RT for 1 h. The reaction progress was monitored by TLC (10% MeOH in DCM). After completion, the reaction was concentrated under reduced pressure. To this residue, 10% NaHCO₃ solution (40 mL) was added and the mixture was extracted with 10% MeOH in DCM (250 mL). The combined organic layer was dried over anhydrous sodium sulfate and filtered, and the filtrate was concentrated under reduced pressure to obtain crude benzyl ((1r,3r)-3-aminocyclobutyl)(isopropyl)carbamate (2.7 g, 91% yield) as a solid. This material was used without further purification. ¹H NMR (400 MHz, DMSO-d₆); δ 7.49-7.28 (m, 5H), 5.08 (s, 2H), 4.46 (quin, J=8.6 Hz, 1H), 3.99 (spt, J=6.8 Hz, 1H), 2.60-2.54 (m, 1H), 1.88-1.50 (m, 4H), 1.13 (d, J=6.7 Hz, 6H), two protons merged with solvent peaks.

### Step 3. Benzyl (1r,3r)-3-(3-hydroxyazetidin-1-yl)cyclobutyl)(isopropyl)carbamate

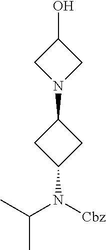 [SMILES: CC(C)N(C(=O)OCc1ccccc1)[C@H]1C[C@H](N2CC(O)C2)C1]

To a stirred solution of benzyl ((1r,3r)-3-aminocyclobutyl)(isopropyl)carbamate (1 g, 3.81 mmol) in isopropanol (20 mL), 2-(chloromethyl) oxirane (0.314 mL, 4.00 mmol) was added at 0° C. and the reaction was stirred at RT for 16 h. The reaction progress was monitored by TLC (10% MeOH in DCM). After completion, the reaction was concentrated under reduced pressure. The residue was re-concentrated from ACN (2×10 mL) then was dissolved in ACN (20 mL). To this mixture, Et₃N (1.594 mL, 11.43 mmol) was added at RT and the reaction was stirred at 80° C. for 16 h. The reaction progress was monitored by TLC (10% MeOH in DCM). After completion, the reaction was concentrated under reduced pressure. The crude product was purified by reverse phase chromatography using a SiliCycle SiliaSep C18 column (40 g) on Biotage isolera one (Mobile phase A: 10 mM NH₄HCO₃ in water, B: ACN) to obtain benzyl ((1r,3r)-3-(3-hydroxyazetidin-1-yl)cyclobutyl) (isopropyl)carbamate (450 mg, 35.5% yield) as a solid. ¹HNMR (400 MHz, DMSO-d₆): δ 7.49-7.15 (m, 5H), 5.22 (d, J=6.5 Hz, 1H), 5.07 (s, 2H), 4.25 (quin, J=8.6 Hz, 1H), 4.17-4.06 (m, 1H), 3.98 (spt, J=6.7 Hz, 1H), 3.45 (dt, J=1.8, 6.0 Hz, 2H), 2.78 (br t, J=6.8 Hz, 1H), 2.59-2.53 (m, 2H), 2.43-2.28 (m, 2H), 1.84-1.73 (m, 2H), 1.11 (d, J=6.8 Hz, 6H); LCMS (Method A): Rt=1.63 min, m/z=319.1 [M+H]⁺, 95.74%.

### Step 4. Benzyl ((1r,3r)-3-(3-fluoroazetidin-1-yl)cyclobutyl)(isopropyl)carbamate

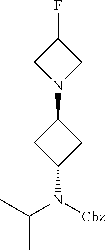 [SMILES: CC(C)N(C(=O)OCc1ccccc1)[C@H]1C[C@H](N2CC(F)C2)C1]

To a stirred solution of benzyl ((1r,3r)-3-(3-hydroxyazetidin-1-yl)cyclobutyl)(isopropyl)carbamate (200 mg, 0.628 mmol) in DCM (6 mL), DAST (0.415 mL, 3.14 mmol) was added at −20° C., and the reaction mixture was stirred at RT for 2 h. The reaction progress was monitored by TLC (10% MeOH in DCM). After completion, the reaction was quenched with ice-cold water (10 mL), and the mixture was extracted with DCM (2×15 mL). The combined organic layer was washed with brine solution (10 mL), dried over anhydrous Na₂SO₄ and filtered, and the filtrate was concentrated under reduced pressure. The crude product was purified by reverse phase chromatography using a SiliCycle SiliaSep C18 column (40 g) on Biotage isolera one (Mobile phase A: 10 mM NH₄HCO₃ in water, B: ACN) to obtain benzyl ((1r,3r)-3-(3-fluoroazetidin-1-yl)cyclobutyl)(isopropyl)carbamate (110 mg, 48.9% yield) as a liquid. ¹H NMR (400 MHz, DMSO-d₆): δ 7.47-7.27 (m, 5H), 5.23-5.01 (m, 3H), 4.25 (quin, J=8.6 Hz, 1H), 3.99 (spt, J=6.7 Hz, 1H), 3.55-3.43 (m, 2H), 3.05-2.84 (m, 3H), 2.46-2.35 (m, 2H), 1.86-1.76 (m, 2H), 1.11 (d, J=6.7 Hz, 6H); LCMS (Method A): Rt=1.99 min, m/z=321.2 [M+H]⁺, 89.52%.

### Step 5. tert-Butyl (3R,4S)-4-fluoro-3-(isopropyl amino) piperidine-1-carboxylate

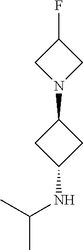

This compound was synthesized following the procedure described for the synthesis of Intermediate 38, step 2, starting with 1 equivalent of benzyl ((1r,3r)-3-(3-fluoroazetidin-1-yl)cyclobutyl)isopropyl)carbamate. After filtration, the filtrate was concentrated under reduced pressure to obtain crude (1r,3r)-3-(3-fluoroazetidin-1-yl)-N-isopropylcyclobutan-1-amine as a colorless liquid. This compound was used without further purification.

Yield: 94%; ¹H NMR (400 MHz, DMSO-d₆): δ 5.25-4.99 (m, 1H), 3.95-3.82 (m, 1H), 3.53-3.43 (m, 2H), 3.39-3.36 (m, 1H), 3.04-2.98 (m, 1H), 2.97-2.91 (m, 2H), 2.79-2.60 (m, 1H), 1.96-1.85 (m, 2H), 1.76-1.64 (m, 2H), 0.93 (d, J=6.3 Hz, 6H); LCMS-ELSD (Method B): Rt=0.28 min, m/z=187.3 [M+H]⁺, 99.87%.

### Step 6. 5-Fluoro-N-((1r,3r)-3-(3-fluoroazetidin-1-yl)cyclobutyl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

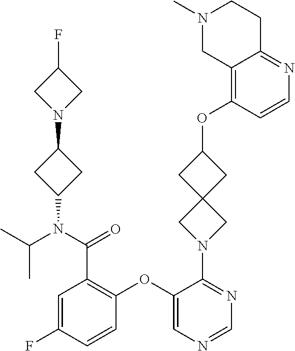

To a stirred solution of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid (80 mg, 0.163 mmol) in DCM (3 mL), (1r,3r)-3-(3-fluoroazetidin-1-yl)-N-isopropylcyclobutan-1-amine (60.6 mg, 0.326 mmol) was added. To this reaction mixture, Et₃N (0.068 mL, 0.488 mmol) and BOP-Cl (62.1 mg, 0.244 mmol) were added at 0° C., and the reaction was stirred at RT for 2 h. The reaction progress was monitored by LCMS. After completion, the reaction was quenched with ice-cold water (5 mL), and the mixture was extracted with DCM (2×10 mL). The combined organic layer was washed with brine solution (5 mL), dried over anhydrous sodium sulfate and filtered, and the filtrate was concentrated under reduced pressure. The crude product was purified by reverse phase chromatography using a SiliCycle SiliaSep C18 column (40 g) on Biotage isolera one (Mobile phase A: 10 mM NH₄HCO₃ in water, B: MeOH) to obtain 5-fluoro-N-((1r,3r)-3-(3-fluoroazetidin-1-yl)cyclobutyl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (14 mg, 12.83% yield) as a solid. ¹HNMR (400 MHz, DMSO-d₆): δ 8.31-8.25 (m, 1H), 8.18 (d, J=5.6 Hz, 11H), 7.79-7.63 (m, 1H), 7.33-7.02 (m, 3H), 6.66 (d, J=5.6 Hz, 1H), 5.29-4.97 (m, 1H), 4.81-4.72 (m, 1H), 4.35-4.08 (m, 5H), 4.07-3.95 (m, 1H), 3.80-3.62 (m, 1H), 3.57-3.46 (m, 1H), 3.17-2.74 (m, 8H), 2.66-2.61 (m, 3H), 2.37 (s, 3H), 2.30-2.20 (m, 2H), 2.17-2.04 (m, 1H), 1.93-1.68 (m, 2H), 1.63-1.52 (m, 1H), 1.47-0.91 (m, 6H); LCMS (Method C): Rt=1.44 min, m/z=660.3 [M+H]⁺; HPLC (Method C): Rt=8.83 min, 98.40%; Chiral SFC (Method H): Rt=3.63 min, 98.87%.

### Example 188. (D1)-5-Fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-((1r,3r)-3-(3-hydroxyazetidin-1-yl)-3-methylcyclobutyl)-N-isopropylbenzamide (Compound No. 188)

### Example 189. (D2)-5-Fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-((1r,3r)-3-(3-hydroxyazetidin-1-yl)-3-methylcyclobutyl)-N-isopropylbenzamide (Compound No. 189)

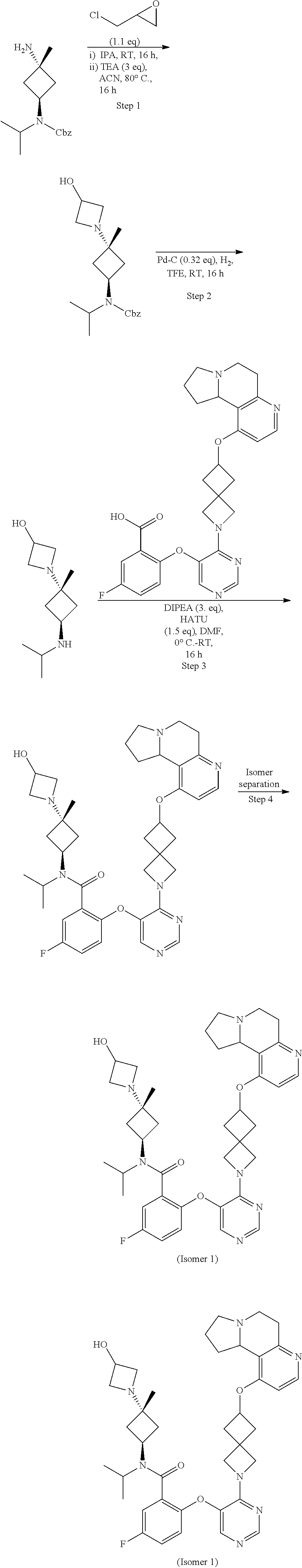

### Step 1. Benzyl ((1r,3r)-3-(3-hydroxyazetidin-1-yl)-3-methylcyclobutyl)(isopropyl)carbamate

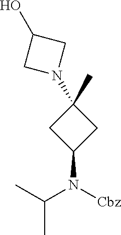 [SMILES: CC(C)N(C(=O)OCc1ccccc1)[C@H]1C[C@](C)(N2CC(O)C2)C1]

This compound was synthesized following the procedure described for the synthesis of Example 187, step 3, starting with 1 equivalent of benzyl ((1r,3r)-3-amino-3-methylcyclobutyl)(isopropyl)carbamate and 1.1 equivalents of 2-(chloromethyl)oxirane. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain benzyl ((1r,3r)-3-(3-hydroxyazetidin-1-yl)-3-methylcyclobutyl)(isopropyl)carbamate a liquid.

Yield: 66.3%; ¹H NMR (400 MHz, DMSO-d₆): δ 7.41-7.30 (m, 5H), 5.20 (d, J=6.8 Hz, 1H), 5.11-5.04 (m, 2H), 4.18-3.93 (m, 2H), 2.81 (br t, J=6.5 Hz, 2H), 2.31-2.25 (m, 1H), 2.19-2.09 (m, 3H), 1.88-1.79 (m, 1H), 1.15-0.95 (m, 9H), two protons merged with solvent peaks; LCMS (Method B): Rt=1.25 min, m/z=333.2 [M+H]⁺, 99.64%.

### Step 2. 1-((1r,3r)-3-(Isopropylamino)-1-methylcyclobutyl)azetidin-3-ol

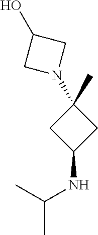

This compound was synthesized following the procedure described for the synthesis of Intermediate 38, step 2, starting with 1 equivalent of benzyl ((1r,3r)-3-(3-hydroxyazetidin-1-yl)-3-methylcyclobutyl)(isopropyl)carbamate, except that 0.32 equivalent of Pd—C was used. After completion, the reaction was diluted with MeOH and filtered through a Celite® pad, and the pad was washed with 10% MeOH in DCM. The filtrate was concentrated under reduced pressure to afford crude to afford crude 1-((1r,3r)-3-(isopropylamino)-1-methylcyclobutyl)azetidin-3-ol as a sticky liquid. This material was used without further purification.

Yield: 45.3%; LCMS-ELSD (Method A): Rt=0.69 min, m/z=199.1 [M+H]⁺, 85.88%.

### Step 3. 5-Fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-((1r,3r)-3-(3-hydroxyazetidin-1-yl)-3-methylcyclobutyl)-N-isopropylbenzamide

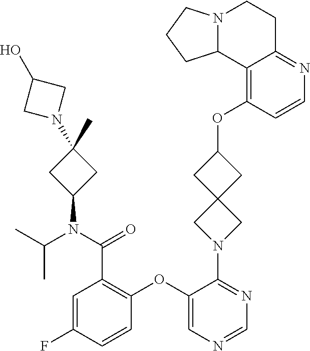 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(N2CC(O)C2)C1]

This compound was synthesized following the procedure described for the synthesis of Example 1, step 2, starting with 1 equivalent of 5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoic acid and 1 equivalent of 1-((1r,3r)-3-(isopropylamino)-1-methylcyclobutyl)azetidin-3-ol. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN), then by achiral prep SFC (Method B) to afford 5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-((1r,3r)-3-(3-hydroxyazetidin-1-yl)-3-methylcyclobutyl)-N-isopropylbenzamide as a solid.

Yield: 21.39%; ¹H NMR (400 MHz, DMSO-d₆): δ 8.32-8.22 (m, 1H), 8.17 (d, J=5.6 Hz, 1H), 7.79-7.60 (m, 1H), 7.32-7.06 (m, 3H), 6.70-6.62 (m, 1H), 5.33-5.16 (m, 1H), 4.75 (quin, J=6.3 Hz, 1H), 4.39-4.02 (m, 6H), 3.79-3.61 (m, 3H), 3.20-3.14 (m, 1H), 3.02-2.74 (m, 8H), 2.73-2.59 (m, 2H), 2.45-2.37 (m, 1H), 2.31-2.01 (m, 4H), 1.86-1.69 (m, 3H), 1.56-0.89 (m, 11H); LCMS (Method H): Rt=1.22 min, m/z=698.4 [M+H]⁺; HPLC (Method C): Rt=7.94 min, 98.45%; Chiral SFC (Method P): Peak 1: Rt=4.16 min, 49.02%; Peak 2: Rt=5.24 min, 47.00%.

### Step 4. (D1)-5-Fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-((1r,3r)-3-(3-hydroxyazetidin-1-yl)-3-methylcyclobutyl)-N-isopropylbenzamide and (D2)-5-fluoro-2-((4-(6-((5,6,8,9,10,10α-hexahydropyrrolo[2,1-f][1,6]naphthyridin-1-yl)ox))-2-azaspiro[3.3]heptan-2-yl)pyrimidin-5-yl)oxy)-N-((1r,3r)-3-(3-hydroxyazetidin-1-yl)-3-methylcyclobutyl)-N-isopropylbenzamide

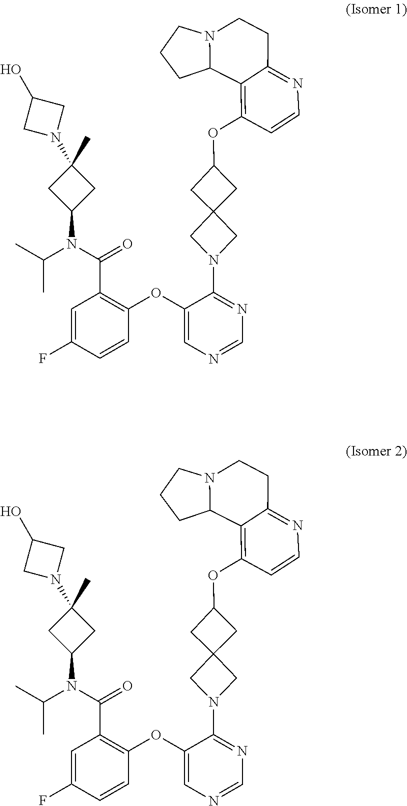 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(N2CC(O)C2)C1 ; CC(C)N(C(=O)c1cc(F)ccc1Oc1cncnc1N1CC2(CC(Oc3ccnc4c3C3CCCN3CC4)C2)C1)[C@H]1C[C@](C)(N2CC(O)C2)C1]

The diastereomeric mixture of 5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-((1r,3r)-3-(3-hydroxyazetidin-1-yl)-3-methylcyclobutyl)-N-isopropylbenzamide was purified by chiral Prep SFC (Method P). The individual diastereomers were then purified again separately, by reverse phase chromatography using a SiliCycle SiliaSep C18 column (40 g) on Biotage isolera one (Mobile phase A: 10 mM NH₄HCO₃ in water, B: ACN), to afford (D1)-5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-((1r,3r)-3-(3-hydroxyazetidin-1-yl)-3-methylcyclobutyl)-N-isopropylbenzamide (26.53 mg, 20.28% yield) and (D2)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-((1r,3r)-3-(3-hydroxyazetidin-1-yl)-3-methylcyclobutyl)-N-isopropylbenzamide (20.01 mg, 15.29% yield) as solids.

Isomer 1. ¹H NMR (400 MHz, DMSO-d₆): δ 8.30-8.22 (m, 1H), 8.19-8.14 (m, 1H), 7.79-7.60 (m, 1H), 7.34-7.04 (m, 3H), 6.73-6.58 (m, 1H), 5.21 (d, J=6.8 Hz, 1H), 4.75 (quin, J=6.4 Hz, 1H), 4.36-3.90 (m, 6H), 3.77-3.60 (m, 3H), 3.20-3.12 (m, 1H), 3.01-2.72 (m, 8H), 2.66-2.60 (m, 2H), 2.45-2.38 (m, 1H), 2.28-2.00 (m, 4H), 1.86-1.66 (m, 3H), 1.54-0.87 (m, 11H); LCMS (Method H): Rt=1.23 min, m/z=698.4 [M+H]⁺; HPLC (Method C): Rt=7.98 min, 99.39%; Chiral SFC (Method P): Rt=4.55 min, 98.05%.

Isomer 2. ¹H NMR (400 MHz, DMSO-d₆): δ 8.30-8.22 (m, 1H), 8.19-8.14 (m, 1H), 7.79-7.60 (m, 1H), 7.34-7.04 (m, 3H), 6.73-6.58 (m, 1H), 5.21 (d, J=6.8 Hz, 1H), 4.75 (quin, J=6.4 Hz, 1H), 4.36-3.90 (m, 6H), 3.77-3.60 (m, 3H), 3.20-3.12 (m, 1H), 3.01-2.72 (m, 8H), 2.66-2.60 (m, 2H), 2.44-2.39 (m, 1H), 2.28-2.00 (m, 4H), 1.86-1.66 (m, 3H), 1.54-0.87 (m, 11H); LCMS (Method H): Rt=1.22 min, m/z=698.4 [M+H]⁺; HPLC (Method C): Rt=7.97 min, 99.35%; Chiral SFC (Method P): Rt=5.39 min, 98.23%.

### Example 190. 5-Fluoro-N-(3-(3-hydroxyazetidin-1-yl)bicyclo[1.1.1]pentan-1-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 190)

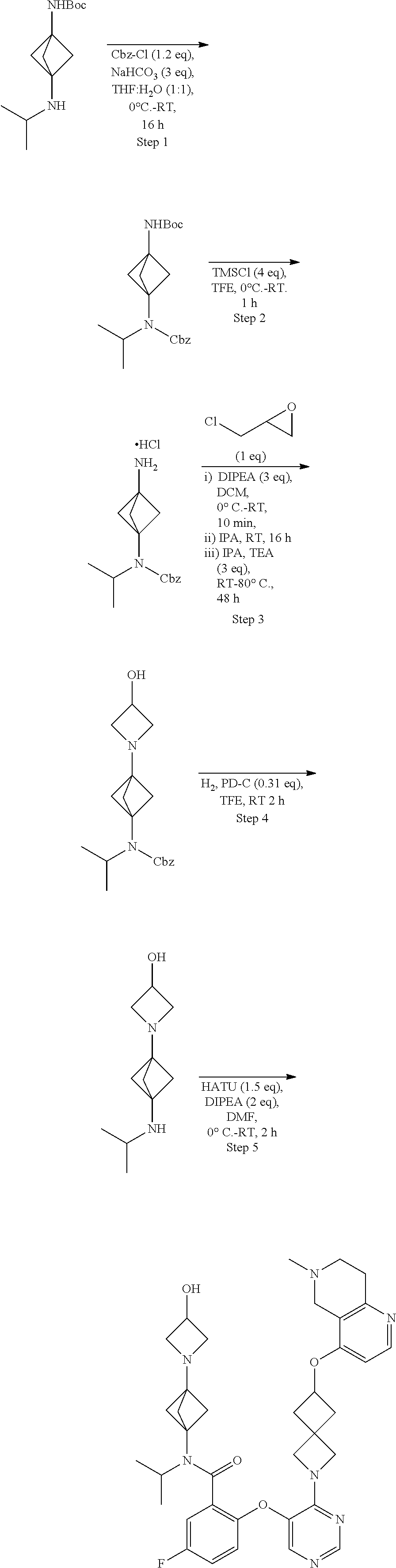

### Step 1. Benzyl (3-((tert-butoxycarbonyl)amino)bicyclo[1.1.1]pentan-1-yl)(isopropyl)carbamate

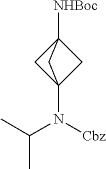 [SMILES: CC(C)N(C(=O)OCc1ccccc1)C12CC(NC(=O)OC(C)(C)C)(C1)C2]

This compound was synthesized following the procedure described for the synthesis of Example 187, step 1, starting with 1 equivalent of tert-butyl (3-(isopropylamino)bicyclo[1.1.1]pentan-1-yl)carbamate, except that 1.2 equivalents of Cbz-Cl was used. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain benzyl (3-((tert-butoxycarbonyl)amino)bicyclo[1.1.1]pentan-1-yl)(isopropyl)carbamate as a solid.

Yield: 54.7%; ¹H NMR (400 MHz, DMSO-CDCl₃): δ 7.40-7.30 (m, 5H), 5.12 (s, 2H), 3.99-3.85 (m, 1H), 2.34 (s, 6H), 1.45 (s, 9H), 1.29 (d, J=7.0 Hz, 6H), one proton merged with solvent peaks.

### Step 2. Benzyl (3-((3-chloro-2-hydroxypropyl)amino)bicyclo[1.1.1]pentan-1-yl)(isopropyl)carbamate Hydrochloride

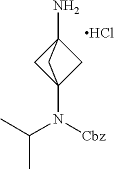 [SMILES: CC(C)N(C(=O)OCc1ccccc1)C12CC(N)(C1)C2 ; Cl]

This compound was synthesized following the procedure described for the synthesis of Example 20, Step 3, starting with 1 equivalent of benzyl (3-((tert-butoxycarbonyl)amino)bicyclo[1.1.1]pentan-1-yl)(isopropyl)carbamate. After completion, the reaction was concentrated under reduced pressure. The residue obtained was triturated with EtOAc, and the supernatant was decanted. The solid was dried under vacuum to obtain crude benzyl (3-aminobicyclo[1.1.1]pentan-1-yl)(isopropyl)carbamate hydrochloride. This material was used without further purification.

Yield: 99%; ¹H NMR (400 MHz, DMSO-d₆): δ 9.04-8.80 (m, 3H), 7.45-7.31 (m, 5H), 5.07 (s, 2H), 3.93-3.73 (m, 1H), 2.27 (s, 6H), 1.22 (d, J=6.9 Hz, 6H).

### Step 3. Benzyl (3-(3-hydroxyazetidin-1-yl)bicyclo[1.1.1]pentan-1-yl)(isopropyl)carbamate

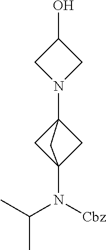 [SMILES: CC(C)N(C(=O)OCc1ccccc1)C12CC(N3CC(O)C3)(C1)C2]

To a stirred solution of benzyl (3-aminobicyclo[1.1.1]pentan-1-yl)(isopropyl)carbamate hydrochloride (1.2 g, 3.86 mmol) in DCM (12 mL), DIPEA (2.023 mL, 11.58 mmol) was added at 0° C. The reaction was stirred at RT for 10 min, then was concentrated under reduced pressure. The residue was dissolved in isopropanol (12 mL), 2-(chloromethyl)oxirane (0.302 mL, 3.86 mmol) was added at 0° C., and the reaction was stirred at RT for 16 h. The reaction progress was monitored by TLC (10% MeOH in DCM). After completion, the reaction was concentrated under reduced pressure. The residue was re-concentrated from ACN (2×10 mL) and was dissolved in ACN (15 mL). To this mixture, TEA (1.633 mL, 12.27 mmol) was added at RT and the reaction was stirred at 80° C. for 48 h. The reaction progress was monitored by LCMS. After completion, the reaction was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN) to obtain benzyl (3-(3-hydroxyazetidin-1-yl)bicyclo[1.1.1]pentan-1-yl)(isopropyl)carbamate (230 mg, 13.23% yield) as a semi-solid. ¹HNMR (400 MHz, DMSO-d₆): δ 7.43-7.29 (m, 5H), 5.05 (s, 2H), 4.20-4.08 (m, 1H), 3.90-3.72 (m, 1H), 3.32-3.28 (m, 2H), 2.84-2.77 (m, 2H), 2.14-1.87 (m, 6H), 1.22 (d, J=6.9 Hz, 6H), one proton merged with solvent peaks; LCMS (Method A): Rt=1.70 min, m/z=331.1 [M+H]⁺, 77.73%.

### Step 4. 1-(3-(Isopropylamino)bicyclo[1.1.1]pentan-1-yl)azetidin-3-ol

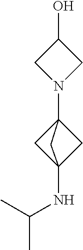

This compound was synthesized following the procedure described for the synthesis of Intermediate 39, step 2, starting with 1 equivalent of benzyl (3-(3-hydroxyazetidin-1-yl)bicyclo[1.1.1]pentan-1-yl)(isopropyl)carbamate, except that 0.31 equivalent of Pd—C was used. After completion, the reaction was filtered through a Celite® pad, and the pad was washed with MeOH. The filtrate was concentrated under reduced pressure to obtain crude 1-(3-(isopropylamino)bicyclo[1.1.1]pentan-1-yl)azetidin-3-ol as a semi-solid. This material was used without further purification.

Yield: 97%; LCMS (Method B): Rt=0.88 min, m/z=197.2 [M+H]⁺, 94.53%.

### Step 5. 5-Fluoro-N-(3-(3-hydroxyazetidin-1-yl)bicyclo[1.1.1]pentan-1-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

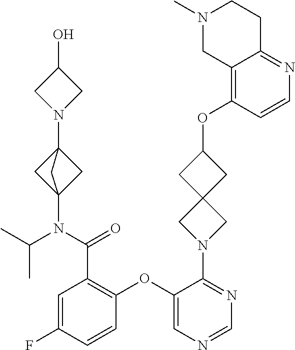 [SMILES: CC(C)N(C(=O)c1cc(F)ccc1Oc1cncnc1N1CC2(CC(Oc3ccnc4c3CN(C)CC4)C2)C1)C12CC(N3CC(O)C3)(C1)C2]

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and two equivalents of 1-(3-(isopropylamino)bicyclo[1.1.1]pentan-1-yl)azetidin-3-ol, except that 2 equivalents of DIPEA was used. After work-up, the crude was purified by Prep HPLC (Method A) to obtain 5-fluoro-N-(3-(3-hydroxyazetidin-1-yl)bicyclo[1.1.1]pentan-1-yl)-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 7.82%; ¹H NMR (400 MHz, DMSO-d₆): δ 8.31-8.27 (m, 1H), 8.18 (d, J=5.5 Hz, 11H), 7.76-7.66 (m, 1H), 7.31-7.20 (m, 2H), 7.07-6.99 (m, 1H), 6.68-6.63 (m, 1H), 5.28 (br s, 1H), 4.74 (quin, J=6.4 Hz, 1H), 4.34-3.76 (m, 6H), 3.37 (s, 2H), 3.29-3.21 (m, 2H), 2.85-2.69 (m, 6H), 2.63 (br t, J=5.8 Hz, 2H), 2.37 (s, 3H), 2.31-2.21 (m, 2H), 2.17 (br s, 2H), 1.75-1.58 (m, 3H), 1.40-1.05 (m, 6H), one proton merged with solvent peaks: LCMS (Method A): Rt=1.51 min, m/z=670.2 [M+H]⁺; HPLC (Method C): Rt=8.17 min, 99.65%.

### Example 191. 5-Fluoro-N-isopropyl-N-((1r,3r)-3-methyl-3-(5-azaspiro 12.31 hexan-5-yl)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 191)

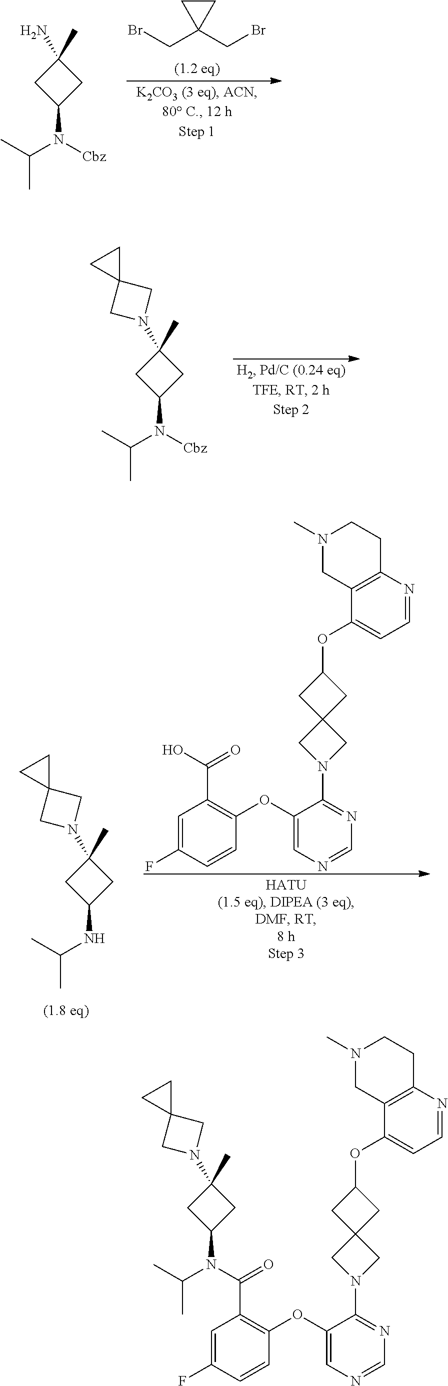

### Step 1. Benzyl isopropyl((1r,3r)-3-methyl-3-(5-azaspiro[2.3]hexan-5-yl)cyclobutyl)carbamate

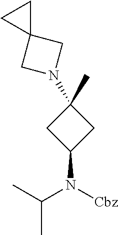 [SMILES: CC(C)N(C(=O)OCc1ccccc1)[C@H]1C[C@](C)(N2CC3(CC3)C2)C1]

In an 8 mL pressure vial, benzyl ((1r,3r)-3-amino-3-methylcyclobutyl)(isopropyl)carbamate (600 mg, 2.171 mmol) was dissolved in ACN (6 mL). To this solution, K₂CO₃ (900 mg, 6.51 mmol) and 1,1-bis(bromomethyl)cyclopropane (594 mg, 2.61 mmol) were added at RT, and the reaction was stirred at 80° C. for 12 h. The reaction progress was monitored by LCMS. After completion, the reaction was quenched with 10% NaHCO₃ solution (15 mL) and the mixture was extracted with EtOAc (3×25 mL). The combined organic layer was washed with brine solution (50 mL), dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 0.1% TFA in water, B: ACN). The pure fractions were concentrated under reduced pressure. The residue was treated with saturated sodium bicarbonate solution (10 mL) and the mixture was extracted with EtOAc (2×25 mL). The combined organic layer was dried over anhydrous sodium sulfate and filtered, and the filtrate was concentrated under reduced pressure to obtain benzyl isopropyl((1r,3r)-3-methyl-3-(5-azaspiro[2.3]hexan-5-yl)cyclobutyl)carbamate (240 mg, 28.7% yield) as a liquid. ¹H NMR (400 MHz, DMSO-d₆): δ 7.55-7.27 (m, 5H), 5.15-5.00 (m, 2H), 4.15-3.95 (m, 2H), 3.23 (s, 4H), 2.25-2.05 (m, 4H), 1.12 (d, J=6.7 Hz, 6H), 1.04 (br s, 3H), 0.48 (s, 4H); LCMS (Method C): Rt=1.99 min, m/z=343.4 [M+H]⁺, 88.94%.

### Step 2. (1r,3r)—N-Isopropyl-3-methyl-3-(5-azaspiro[2.3]hexan-5-yl)cyclobutan-1-amine

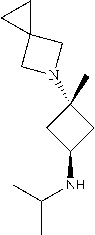

This compound was synthesized following the procedure described for the synthesis of Intermediate 39, step 2, starting with 1 equivalent of benzyl isopropyl((1r,3r)-3-methyl-3-(5-azaspiro[2.3]hexan-5-yl)cyclobutyl)carbamate, except that 0.24 equivalents of Pd—C was used. After filtration, the filtrate was concentrated under reduced pressure to obtain crude (1r,3r)—N-isopropyl-3-methyl-3-(5-azaspiro[2.3]hexan-5-yl)cyclobutan-1-amine as a liquid. This material was used without further purification.

Quantitative yield; LCMS (Method B): Rt=0.42 min, m/z=209.2 [M+H]⁺, 99.15%.

### Step 3. 5-Fluoro-N-isopropyl-N-((1r,3r)-3-methyl-3-(5-azaspiro[2.3]hexan-5-yl)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

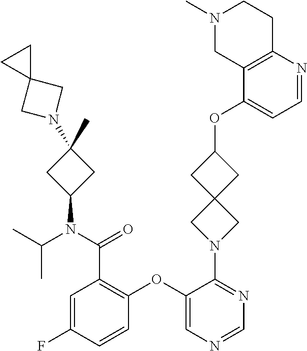 [SMILES: CC(C)N(C(=O)c1cc(F)ccc1Oc1cncnc1N1CC2(CC(Oc3ccnc4c3CN(C)CC4)C2)C1)[C@H]1C[C@](C)(N2CC3(CC3)C2)C1]

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.8 equivalents of (1r,3r)—N-isopropyl-3-methyl-3-(5-azaspiro[2.3]hexan-5-yl)cyclobutan-1-amine. After work-up, the crude was purified by reverse phase chromatography using a SiliCycle SiliaSep C18 column (40 g) on Biotage isolera one (Mobile phase A: 10 mM NH₄HCO₃ in water, B: ACN) to obtain 5-fluoro-N-isopropyl-N-((1r,3r)-3-methyl-3-(5-azaspiro[2.3]hexan-5-yl)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 8.80%; ¹H NMR (400 MHz, DMSO-d₆): δ 8.31-8.25 (m, 1H), 8.18 (d, J=5.5 Hz, 1H), 7.80-7.62 (m, 1H), 7.36-7.06 (m, 3H), 6.71-6.61 (m, 1H), 4.75 (quin, J=6.5 Hz, 1H), 4.42-3.97 (m, 4H), 3.87-3.60 (m, 2H), 3.36 (br s, 2H), 3.28-3.21 (m, 2H), 3.12-3.05 (m, 1H), 2.88-2.72 (m, 5H), 2.63 (br t, J=5.7 Hz, 2H), 2.36 (s, 3H), 2.30-2.21 (m, 2H), 2.18-1.97 (m, 2H), 1.89-1.77 (m, 1H), 1.51-0.89 (m, 9H), 0.60-0.32 (m, 4H), one proton merged with solvent peaks; LCMS (Method A): Rt=1.79 min, m/z=682.2 [M+H]⁺; HPLC (Method G): Rt=6.45 min, 97.65%; Chiral SFC (Method D): Rt=1.73 min, 100%.

### Example 192. 5-Fluoro-N-isopropyl-N-((1r,3r)-3-methyl-3-(3-methylazetidin-1-yl)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 192)

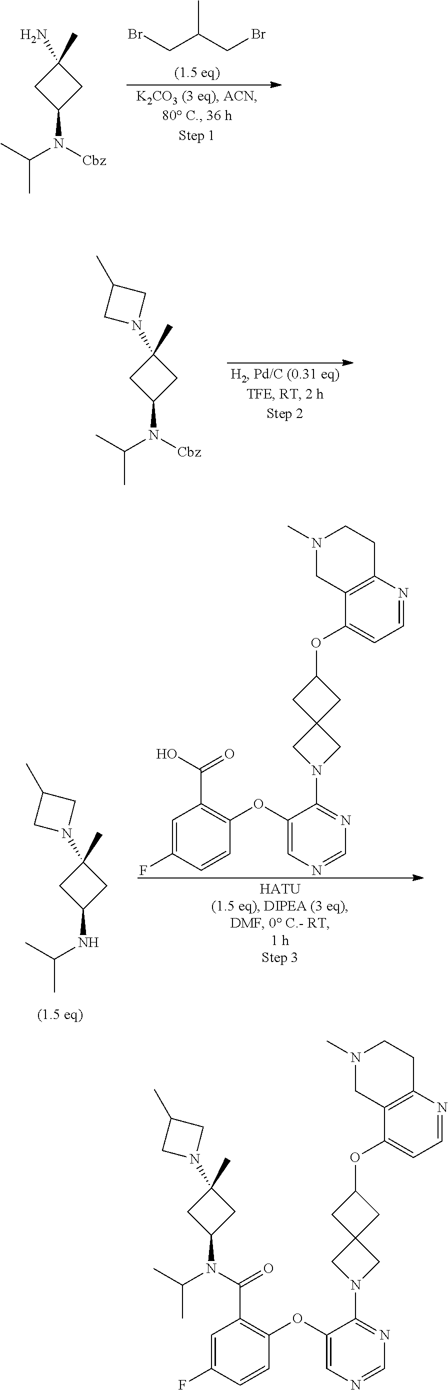

### Step 1. Benzyl isopropyl((1r,3r)-3-methyl-3-(3-methylazetidin-1-yl)cyclobutyl)carbamate

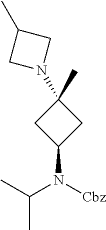 [SMILES: CC1CN([C@]2(C)C[C@@H](N(C(=O)OCc3ccccc3)C(C)C)C2)C1]

Step 1 was performed following the same procedure in two batches.

This compound was synthesized following the procedure described for the synthesis of Intermediate 40, step 1, starting with 1 equivalent of benzyl ((1r,3r)-3-amino-3-methylcyclobutyl)(isopropyl)carbamate and 1.5 equivalents of 1,3-dibromo-2-methylpropane. After completion, the reaction was quenched with 10% aqueous NaHCO₃, and the mixture was extracted with EtOAc. The combined organic layer was dried over anhydrous sodium sulfate and filtered, and the filtrate was concentrated under reduced pressure. The crude from both batches were combined and purified by reverse phase chromatography using a SiliCycle SiliaSep C18 column (80 g) on Biotage isolera one (Mobile phase A: 0.1% TFA in water, B: ACN). The pure fractions were concentrated under reduced pressure. The residue was treated with saturated sodium bicarbonate solution and the mixture was extracted with DCM. The combined organic layer was dried over anhydrous sodium sulfate and filtered, and the filtrate was concentrated under reduced pressure to obtain benzyl isopropyl((1r,3r)-3-methyl-3-(3-methylazetidin-1-yl)cyclobutyl)carbamate as a gummy liquid.

Yield: 17.9%; ¹H NMR (400 MHz, DMSO-d₆): δ 7.44-7.30 (m, 5H), 5.08 (s, 2H), 3.93-3.83 (m, 2H), 3.24 (t, J=6.9 Hz, 2H), 2.68 (t, J=6.4 Hz, 2H), 2.29-2.39 (m, 1H), 2.11 (br s, 2H), 2.09 (br s, 2H), 1.12 (d, J=6.9 Hz, 6H), 1.09 (d, J=6.8 Hz, 3H), 0.97 (br s, 3H); LCMS (Method H): Rt=1.69 min, m/z=331.2 [M+H]⁺, 97.60%.

### Step 2. Benzyl isopropyl((1r,3r)-3-methyl-3-(3-methylazetidin-1-yl)cyclobutyl)carbamate

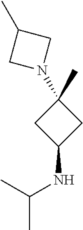

Step 2 was performed following the same procedure in two batches.

This compound was synthesized following the procedure described for the synthesis of Intermediate 38, Step 2, starting with 1 equivalent of benzyl isopropyl((1r,3r)-3-methyl-3-(3-methylazetidin-1-yl)cyclobutyl)carbamate, except that 0.31 equivalent of Pd—C was used. After filtration, the filtrate from both batches were combined and concentrated under reduced pressure to obtain crude (1r,3r)—N-isopropyl-3-methyl-3-(3-methylazetidin-1-yl)cyclobutan-1-amine as a liquid. This compound was used without further purification.

Quantitative yield; ¹H NMR (400 MHz, DMSO-d₆): δ 3.22 (t, J=7.1 Hz, 2H), 3.18-3.08 (m, 1H), 2.75-2.63 (m, 3H), 2.38-2.29 (m, 1H), 2.27-2.20 (m, 2H), 1.45-1.38 (m, 2H), 1.09 (d, J=6.8 Hz, 3H), 1.04 (s, 3H), 0.94 (d, J=6.3 Hz, 6H), one proton merged with solvent peak.

### Step 3. 5-Fluoro-N-isopropyl-N-((1r,3r)-3-methyl-3-(3-methylazetidin-1-yl)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

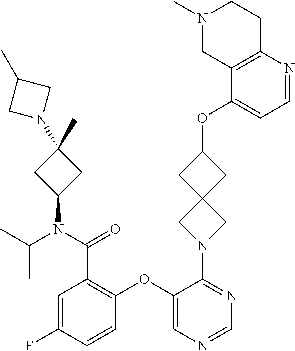 [SMILES: CC1CN([C@]2(C)C[C@@H](N(C(=O)c3cc(F)ccc3Oc3cncnc3N3CC4(CC(Oc5ccnc6c5CN(C)CC6)C4)C3)C(C)C)C2)C1]

This compound was synthesized following the procedure described for the synthesis of Example 1, Step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.5 equivalents of (1r,3r)—N-isopropyl-3-methyl-3-(3-methylazetidin-1-yl)cyclobutan-1-amine. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain 5-fluoro-N-isopropyl-N-((1r,3r)-3-methyl-3-(3-methylazetidin-1-yl)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 30.4%; ¹H NMR (400 MHz, DMSO-d₆): δ 8.27 (s, 1H), 8.18 (d, J=5.5 Hz, 1H), 7.82-7.55 (m, 1H), 7.34-7.04 (m, 3H), 6.73-6.60 (m, 1H), 4.75 (quin, J=6.4 Hz, 1H), 4.45-4.09 (m, 4H), 3.81-3.59 (m, 2H), 3.36 (s, 2H), 3.29-3.23 (m, 1H), 3.11-3.05 (m, 1H), 2.87-2.70 (m, 5H), 2.63 (br t, J=5.8 Hz, 2H), 2.36 (s, 3H), 2.30-2.21 (m, 3H), 2.17-1.93 (m, 2H), 1.85-1.72 (m, 1H), 1.49-0.86 (m, 12H), two protons merged with solvent peaks; LCMS (Method A): Rt=1.72 min, m/z=670.2 [M+H]⁺; HPLC (Method C): Rt=8.78 min, 98.48%; Chiral SFC (Method H): Rt=4.17 min, 96.38%.

### Example 193. 5-Fluoro-N-isopropyl-N-((1r,3r)-3-methyl-3-(2-azaspiro[3.3]heptan-2-yl)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 193)

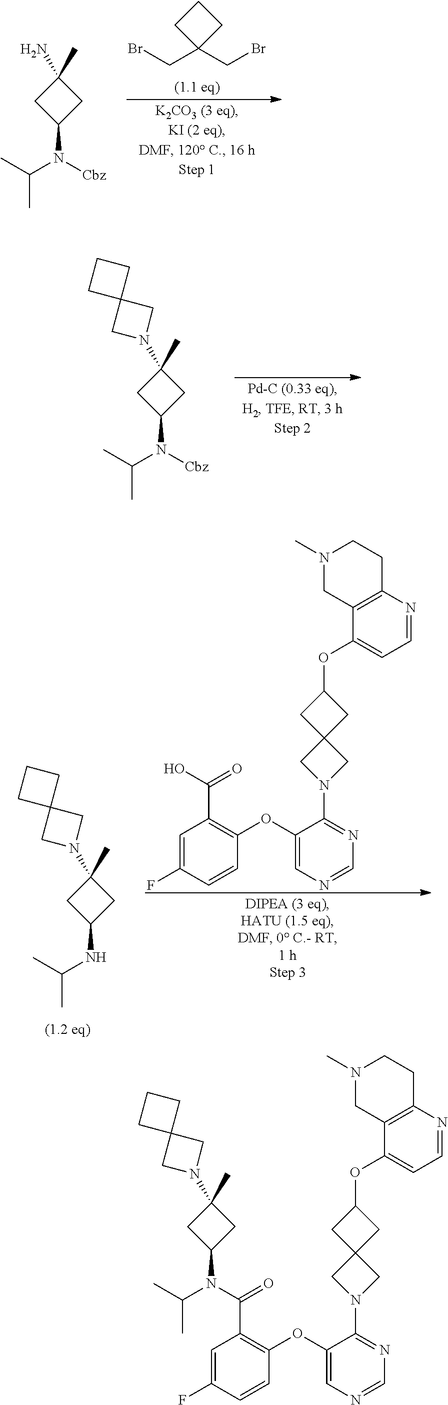

### Step 1. Benzyl isopropyl((1r,3r)-3-methyl-3-(2-azaspiro[3.3]heptan-2-yl)cyclobutyl)carbamate

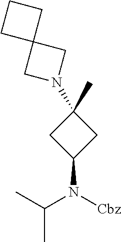 [SMILES: CC(C)N(C(=O)OCc1ccccc1)[C@H]1C[C@](C)(N2CC3(CCC3)C2)C1]

To a stirred solution of benzyl ((1r,3r)-3-amino-3-methylcyclobutyl)(isopropyl)carbamate (1 g, 3.62 mmol) in DMF (15 mL), KI (1.201 g, 7.24 mmol) and K₂CO₃ (1.5 g, 10.85 mmol) were added at RT under a nitrogen atmosphere. To this solution, 1,1-bis(bromomethyl)cyclobutane (0.963 g, 3.98 mmol) was added at RT. The reaction was stirred at 120° C. for 16 h. The reaction was monitored by LCMS. After completion, the reaction was diluted with water (50 mL) and the mixture was extracted with EtOAc (2×30 mL). The combined organic layer was washed with brine solution (50 mL), dried over anhydrous sodium sulfate and filtered, and the filtrate was concentrated under reduced pressure. The crude was purified by silica gel flash column chromatography using MeOH in DCM (product eluted at 12% MeOH in DCM) to obtain benzyl isopropyl((1r,3r)-3-methyl-3-(2-azaspiro[3.3]heptan-2-yl)cyclobutyl)carbamate (0.75 g, 55.9% yield) as a gummy liquid. ¹H NMR (400 MHz, DMSO-d₆): δ 7.43-7.29 (m, 5H), 5.08 (s, 2H), 4.10-3.92 (m, 2H), 3.20 (br s, 4H), 2.22-2.00 (m, 7H), 1.76 (quin, J=7.6 Hz, 2H), 1.10 (d, J=6.8 Hz, 6H), 1.00 (s, 3H), one proton merged with solvent peaks; LCMS (Method B): Rt=1.50 min, m/z=357.2 [M+H]⁺, 96.13%.

### Step 2. (1r,3r)—N-Isopropyl-3-methyl-3-(2-azaspiro[3.3]heptan-2-yl)cyclobutan-1-amine

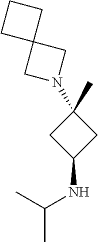 [SMILES: CC(C)N[C@H]1C[C@](C)(N2CC3(CCC3)C2)C1]

This compound was synthesized following the procedure described for the synthesis of Intermediate 38, step 2, starting with 1 equivalent of benzyl isopropyl((1r,3r)-3-methyl-3-(2-azaspiro[3.3]heptan-2-yl)cyclobutyl)carbamate, except that 0.33 equivalent of Pd—C was used. After filtration, the filtrate was concentrated under reduced pressure to obtain crude ((1r,3r)—N-isopropyl-3-methyl-3-(2-azaspiro[3.3]heptan-2-yl)cyclobutan-1-amine as a semisolid. This material was used without further purification.

Yield: 52.6%; LCMS-ELSD (Method A): Rt=1.31 min, m/z=223.1 [M+H]⁺, 53.50%.

### Step 3. 5-Fluoro-N-isopropyl-N-((1r,3r)-3-methyl-3-(2-azaspiro[3.3]heptan-2-yl)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

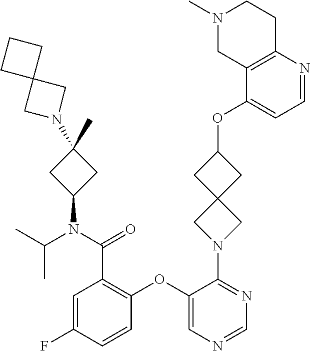 [SMILES: CC(C)N(C(=O)c1cc(F)ccc1Oc1cncnc1N1CC2(CC(Oc3ccnc4c3CN(C)CC4)C2)C1)[C@H]1C[C@](C)(N2CC3(CCC3)C2)C1]

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.2 equivalents of (1r,3r)—N-isopropyl-3-methyl-3-(2-azaspiro[3.3]heptan-2-yl)cyclobutan-1-amine. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN), then by Prep HPLC (Method L), and then by achiral Prep SFC (Method D) to obtain 5-fluoro-N-isopropyl-N-((1r,3r)-3-methyl-3-(2-azaspiro[3.3]heptan-2-yl)cyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 14.60%; ¹H NMR (400 MHz, DMSO-d₆): δ 8.27 (s, 1H), 8.20-8.15 (m, 1H), 7.77-7.61 (m, 1H), 7.32-7.06 (m, 31H), 6.69-6.60 (m, 1H), 4.74 (quin, J=6.1 Hz, 1H), 4.36-3.90 (m, 4H), 3.77-3.61 (m, 2H), 3.36 (br s, 2H), 3.08 (br s, 2H), 2.87 (s, 2H), 2.84-2.74 (m, 5H), 2.63 (br t, J=5.7 Hz, 2H), 2.36 (s, 3H), 2.25 (br s, 2H), 2.15-1.89 (m, 5H), 1.84-1.65 (m, 3H), 1.47-1.23 (m, 3H), 1.19-0.89 (m, 7H); LCMS (Method A): Rt=1.86 min, m/z=696.2 [M+H]⁺; HPLC (Method C): Rt=9.44 min, 99.19%; Chiral SFC (Method H): Rt=4.44 min, 99.24%.

### Example 194. N-((1r,3r)-3-(Azetidin-1-yl)-3-methylcyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 194)

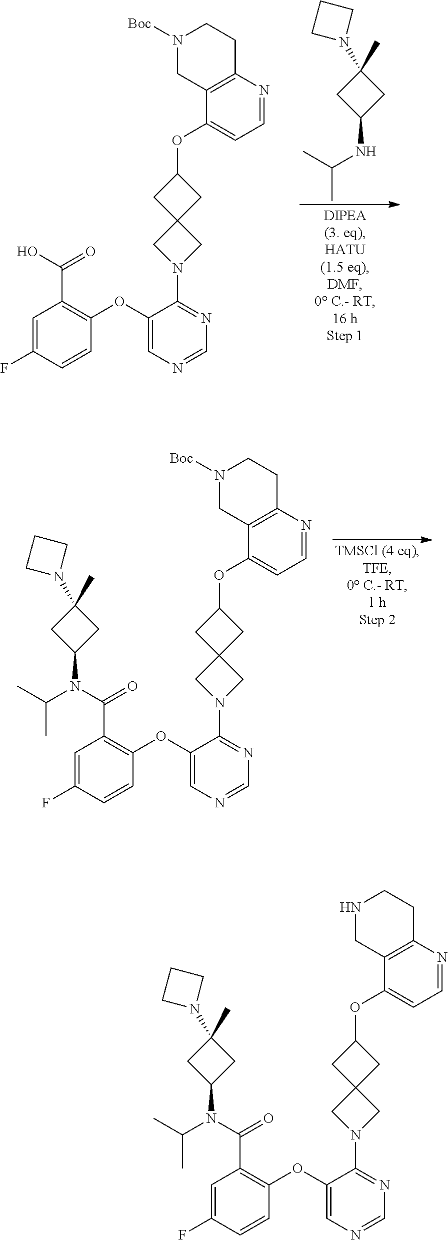 [SMILES: CC(C)(C)OC(=O)N1CCc2nccc(OC3CC4(C3)CN(c3ncncc3Oc3ccc(F)cc3C(=O)O)C4)c2C1 ; CC(C)N[C@H]1C[C@](C)(N2CCC2)C1 ; CC(C)N(C(=O)c1cc(F)ccc1Oc1cncnc1N1CC2(CC(Oc3ccnc4c3CN(C(=O)OC(C)(C)C)CC4)C2)C1)[C@H]1C[C@](C)(N2CCC2)C1 ; CC(C)N(C(=O)c1cc(F)ccc1Oc1cncnc1N1CC2(CC(Oc3ccnc4c3CNCC4)C2)C1)[C@H]1C[C@](C)(N2CCC2)C1]

### Step 1. tert-Butyl 4-((2-(5-(2-(((1r,3r)-3-(azetidin-1-yl)-3-methylcyclobutyl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate

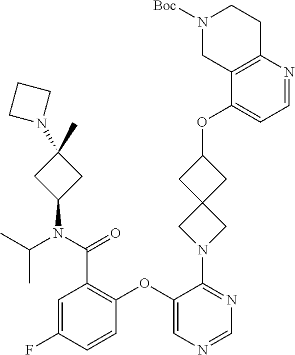 [SMILES: CC(C)N(C(=O)c1cc(F)ccc1Oc1cncnc1N1CC2(CC(Oc3ccnc4c3CN(C(=O)OC(C)(C)C)CC4)C2)C1)[C@H]1C[C@](C)(N2CCC2)C1]

This compound was synthesized following the procedure described for the synthesis of Example 1, step 2, starting with 1 equivalent of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1 equivalent of (1r,3r)-3-(azetidin-1-yl)-N-isopropyl-3-methylcyclobutan-1-amine. After completion, the reaction was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure to afford tert-butyl 4-((2-(5-(2-(((1r,3r)-3-(azetidin-1-yl)-3-methylcyclobutyl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate as a solid.

Yield: 27.1%; LCMS (Method A): Rt=1.97 min, m/z=742.7 [M+H]⁺, 91.74%.

### Step 2. N-((1r,3r)-3-(Azetidin-1-yl)-3-methylcyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide

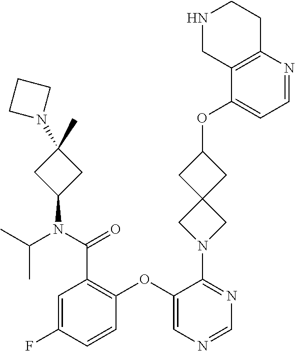 [SMILES: CC(C)N(C(=O)c1cc(F)ccc1Oc1cncnc1N1CC2(CC(Oc3ccnc4c3CNCC4)C2)C1)[C@H]1C[C@](C)(N2CCC2)C1]

To a stirred solution of tert-butyl 4-((2-(5-(2-(((1r,3r)-3-(azetidin-1-yl)-3-methylcyclobutyl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate (0.240 g, 0.323 mmol) in TFE (3 mL), TMSCl (0.165 mL, 1.294 mmol) at 0° C. The reaction was stirred at RT for 1 h. After completion, the reaction was concentrated under reduced pressure. The residue was diluted with saturated sodium bicarbonate solution (10 mL) and the mixture was extracted with 10% MeOH in DCM (3×10 mL). The combined organic layer was dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN), then by chiral Prep SFC (Method N). The pure fractions were concentrated under reduced pressure and lyophilized to afford N-((1r,3r)-3-(azetidin-1-yl)-3-methylcyclobutyl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide (64.63 mg, 30.4% yield) as a solid. ¹H NMR (400 MHz, DMSO-d₆): δ 8.31-8.23 (m, 1H), 8.18-8.13 (m, 1H), 7.79-7.59 (m, 1H), 7.34-7.04 (m, 3H), 6.69-6.55 (m, 1H), 4.72 (quin, J=6.2 Hz, 1H), 4.37-3.92 (m, 4H), 3.80-3.62 (m, 3H), 3.11 (br t, J=6.4 Hz, 2H), 2.98-2.88 (m, 4H), 2.77 (br s, 2H), 2.68 (br s, 2H), 2.29-1.97 (m, 4H), 1.94-1.68 (m, 4H), 1.48-0.86 (m, 9H), two protons merged with solvent peaks; LCMS (Method H): Rt=1.00 min, m/z=642.3 [M+H]⁺; HPLC (Method F): Rt=5.85 min, 98.65%; Chiral SFC (Method J): Rt=5.20 min, 96.87%.

### Example 195. N-((1r,3r)-3-(3,3-Dimethylazetidin-1-yl)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 195)

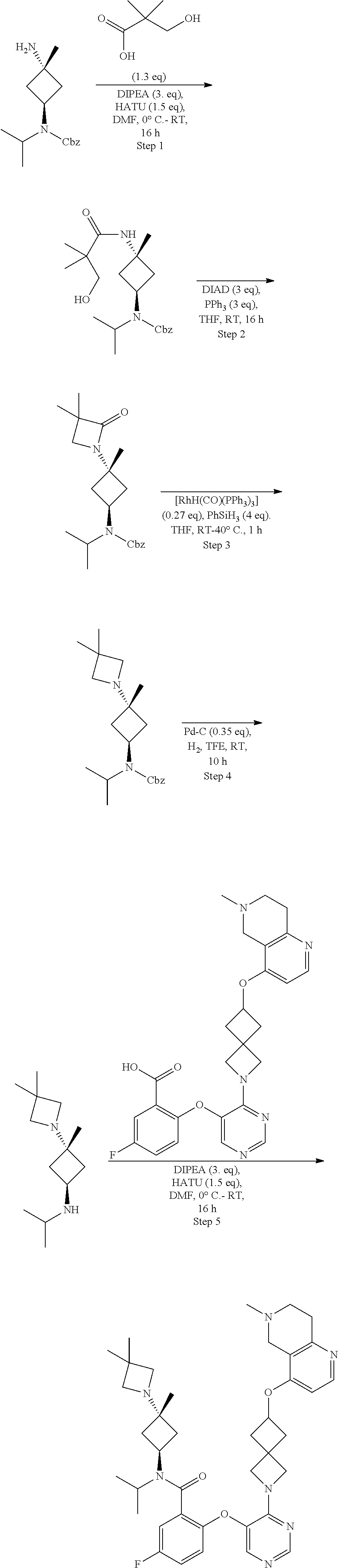

### Step 1. Benzyl ((1r,3r)-3-(3-hydroxy-2,2-dimethylpropanamido)-3-methylcyclobutyl)(isopropyl)carbamate

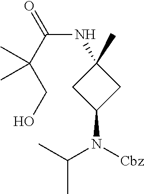 [SMILES: CC(C)N(C(=O)OCc1ccccc1)[C@H]1C[C@](C)(NC(=O)C(C)(C)CO)C1]

To a stirred solution of benzyl ((1r,3r)-3-amino-3-methylcyclobutyl)(isopropyl)carbamate (1 g, 3.62 mmol) in DMF (10 mL), 3-hydroxy-2,2-dimethylpropanoic acid (0.556 g, 4.70 mmol) and DIPEA (1.891 mL, 10.85 mmol) were added at RT. To this reaction mixture, HATU (2.064 g, 5.43 mmol) was added at 0° C. The reaction was stirred at RT for 16 h. The reaction was monitored by LCMS and TLC (10% MeOH in DCM). After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain benzyl ((1r,3r)-3-(3-hydroxy-2,2-dimethylpropanamido)-3-methylcyclobutyl)(isopropyl)carbamate (900 mg, 58.1% yield) as a solid. LCMS (Method A): Rt=1.89 min, m/z=377.2 [M+H]⁺, 88.48%.

### Step 2. Benzyl ((1r,3r)-3-(3,3-dimethyl-2-oxoazetidin-1-yl)-3-methylcyclobutyl)(isopropyl)carbamate

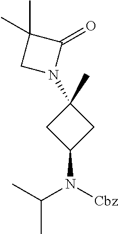 [SMILES: CC(C)N(C(=O)OCc1ccccc1)[C@H]1C[C@](C)(N2CC(C)(C)C2=O)C1]

To a stirred solution of benzyl ((1r,3r)-3-(3-hydroxy-2,2-dimethylpropanamido)-3-methylcyclobutyl)(isopropyl)carbamate (1.3 g, 3.45 mmol) and triphenylphosphine (2.72 g, 10.36 mmol) in THF (15 mL), DIAD (2.054 mL, 10.36 mmol) was added at RT. The reaction was stirred at RT for 16 h. The reaction progress was monitored by TLC (20% EtOAc in hexane). After completion, the reaction was quenched with ice-water (50 mL), and the mixture was extracted with EtOAc (3×70 mL). The combined organic layer was dried over anhydrous sodium sulfate and filtered, and the filtrate was concentrated under reduced pressure. The crude product was purified by silica gel flash column chromatography using EtOAc in hexane (product eluted at 10-20% EtOAc in hexane), and then by reverse phase chromatography using a SiliCycle SiliaSep C18 column (40 g) on Biotage isolera one (Mobile phase A: 10 mM NH₄HCO₃ in water, B: ACN), to afford benzyl ((1r,3r)-3-(3,3-dimethyl-2-oxoazetidin-1-yl)-3-methylcyclobutyl)(isopropyl)carbamate (500 mg, 36.4% yield) as a sticky liquid. LCMS (Method A): Rt=2.19 min, m/z=359.1 [M+H]⁺, 90.20%.

### Step 3. Benzyl ((1r,3r)-3-(3,3-dimethylazetidin-1-yl)-3-methylcyclobutyl)(isopropyl)carbamate

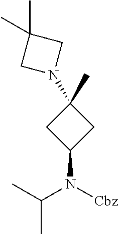 [SMILES: CC(C)N(C(=O)OCc1ccccc1)[C@H]1C[C@](C)(N2CC(C)(C)C2)C1]

To a solution of benzyl ((1r,3r)-3-(3,3-dimethyl-2-oxoazetidin-1-yl)-3-methylcyclobutyl)(isopropyl)carbamate (200 mg, 0.558 mmol) in THF (0.4 mL), carbonylhydridetris(triphenylphosphine)rhodium(I) (138 mg, 0.151 mmol) and phenylsilane (241 mg, 2.232 mmol) were added dropwise at RT. The reaction was stirred at 40° C. for 1 h. After completion, the reaction was cooled to RT and quenched in ice water (10 mL), and the mixture was extracted with EtOAc (2×10 mL). The combined organic layer was dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM FA in water, B: ACN) to obtain benzyl ((1r,3r)-3-(3,3-dimethylazetidin-1-yl)-3-methylcyclobutyl)(isopropyl)carbamate (100 mg, 39.8% yield) as a sticky liquid. LCMS (Method B): Rt=1.56 min, m/z=345.2 [M+H]⁺, 76.42%.

### Step 4. (1r,3r)-3-(3,3-Dimethylazetidin-1-yl)-N-isopropyl-3-methylcyclobutan-1-amine

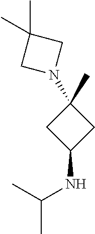

This compound was synthesized following the procedure described for the synthesis of Example 38, step 2, starting with 1 equivalent of benzyl ((1r,3r)-3-(3,3-dimethylazetidin-1-yl)-3-methylcyclobutyl)(isopropyl)carbamate, except that 0.35 equivalent of Pd—C was used. After filtration, the filtrate was concentrated under reduced pressure to afford crude (1r,3r)-3-(3,3-dimethylazetidin-1-yl)-N-isopropyl-3-methylcyclobutan-1-amine as a gummy compound. This material was used without further purification.

Yield: Quantitative: LCMS-ELSD (Method B): Rt=0.41 min, m/z=211.2 [M+H]⁺, 93.51%.

### Step 5. N-((1r,3r)-3-(3,3-Dimethylazetidin-1-yl)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

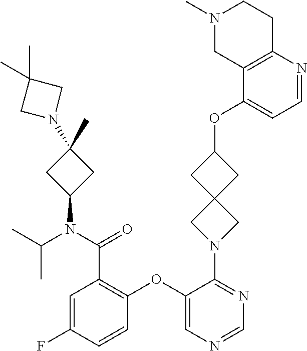 [SMILES: CC(C)N(C(=O)c1cc(F)ccc1Oc1cncnc1N1CC2(CC(Oc3ccnc4c3CN(C)CC4)C2)C1)[C@H]1C[C@](C)(N2CC(C)(C)C2)C1]

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1 equivalent of (1r,3r)-3-(3,3-dimethylazetidin-1-yl)-N-isopropyl-3-methylcyclobutan-1-amine. After work-up, the crude compound was purified by reverse phase chromatography using a SiliCycle SiliaSep C18 column (60 g) on Biotage isolera one (Mobile phase A: 10 mM NH₄HCO₃ in water, B: ACN), then by achiral Prep SFC (Method D). The pure fractions were concentrated under reduced pressure and lyophilized to afford N-((1r,3r)-3-(3,3-dimethylazetidin-1-yl)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 2.84%; ¹H NMR (400 MHz, DMSO-d₆): δ 8.27 (d, J=3.9 Hz, 1H), 8.18 (br d, J=5.5 Hz, 1H), 7.79-7.59 (m, 1H), 7.33-7.05 (m, 3H), 6.70-6.59 (m, 1H), 4.80-4.69 (m, 1H), 4.34-4.08 (m, 4H), 4.02-3.64 (m, 2H), 3.36 (br s, 2H), 2.86 (s, 2H), 2.84-2.73 (m, 5H), 2.66-2.58 (m, 4H), 2.36 (s, 3H), 2.30-2.21 (m, 2H), 2.18-1.92 (m, 2H), 1.85-1.71 (m, 1H), 1.48-1.29 (m, 3H), 1.25-0.91 (m, 12H); LCMS (Method H): Rt=1.08 min, m/z=684.4 [M+H]⁺; HPLC (Method C): Rt=9.40 min, 98.64%; Chiral SFC (Method H): Rt=4.12 min, 98.82%.

### Example 196. N-((1r,3r)-3-(3-(Ethoxy methyl)azetidin-1-yl)-3-methylcyclobutyl)-5-fluoro-N-isopropyl-2-((4-(6-((6-methyl-5,6,7,8-tetrahydro-1,6-naphthyridin-4-yl)oxy)-2-azaspiro 13.31 heptan-2-yl)pyrimidin-5-yl)oxy)benzamide (Compound No. 196)

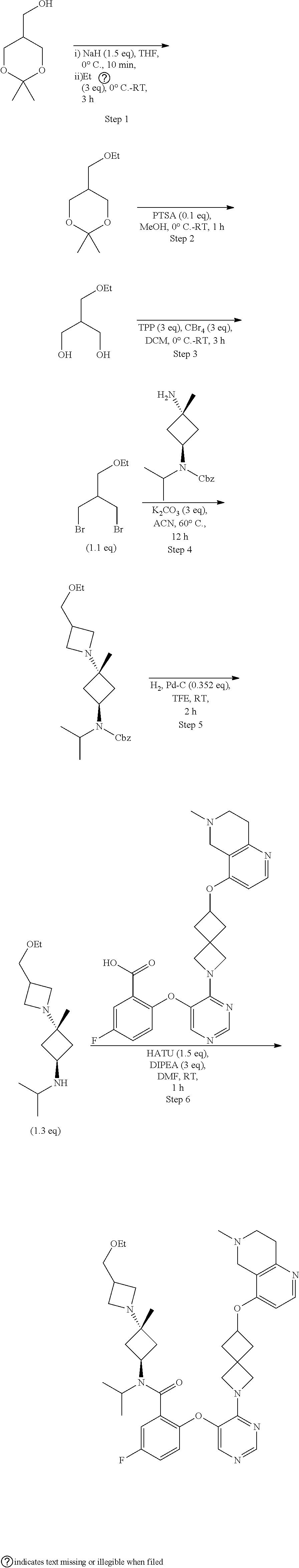

### Step 1. 5-(Ethoxy methyl)-2,2-dimethyl-1,3-dioxane

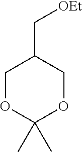

To a stirred solution of (2,2-dimethyl-1,3-dioxan-5-yl) methanol (3 g, 20.52 mmol) in THF (60 mL) under a nitrogen atmosphere, NaH (1.231 g, 30.8 mmol) was added and the reaction was stirred at 0° C. for 10 min. To this reaction mixture, iodoethane (4.98 mL, 61.6 mmol) was added at 0° C. and the reaction was stirred for at RT 3 h. The reaction progress was monitored by TLC (20% EtOAc in hexane). After completion, the reaction was quenched with ice-cold water (100 mL), and the mixture was extracted with EtOAc (2×50 mL). The combined organic layer was dried over anhydrous sodium sulfate and filtered, and the filtrate was concentrated under reduced pressure. The crude was purified by silica gel flash column chromatography using EtOAc in hexane (product eluted at 15% EtOAc in hexane) to obtain 5-(ethoxy methyl)-2,2-dimethyl-1,3-dioxane (2.28 g, 63.8% yield) as a liquid. ¹H NMR (400 MHz, DMSO-d₆): δ 3.84 (dd, J=4.2, 11.7 Hz, 2H), 3.61 (dd, J=6.9, 11.6 Hz, 2H), 3.47-3.25 (m, 4H), 1.92-1.77 (m, 1H), 1.31 (d, J=4.6 Hz, 6H), 1.10 (t, J=7.0 Hz, 3H).

### Step 2. 2-(Ethoxy methyl) propane-1,3-diol

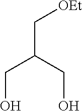

To a stirred solution of 5-(ethoxy methyl)-2,2-dimethyl-1,3-dioxane (2.28 g, 13.09 mmol) in MeOH (23 mL), PTSA (0.249 g, 1.309 mmol) was added at 0° C., and the reaction was stirred at RT for 1 h. The reaction progress was monitored by TLC (10% MeOH in DCM). After completion, the reaction was neutralized with triethylamine (pH˜7), and the mixture was concentrated under reduced pressure. The crude was purified by silica gel flash column chromatography using MeOH in DCM (product eluted at 5% MeOH in DCM) to obtain 2-(ethoxy methyl) propane-1,3-diol (1.3 g, 74.0% yield) as a liquid. ¹H NMR (400 MHz, DMSO-d₆): δ 4.34 (br s, 2H), 3.51-3.29 (m, 8H), 1.73 (spt, J=5.9 Hz, 1H), 1.09 (t, J=7.0 Hz, 3H).

### Step 3. 1,3-Dibromo-2-(ethoxy methyl) propane

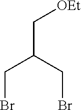

In a dried, 100 mL two-neck round bottom flask under a nitrogen atmosphere, 2-(ethoxy methyl) propane-1,3-diol (1.6 g, 11.92 mmol) was dissolved in DCM (30 mL). To this solution, triphenylphosphine (7.86 g, 30.0 mmol) and CBr₄(9.94 g, 30.0 mmol) were added at 0° C. and the reaction was stirred at RT for 3 h. The reaction progress was monitored by TLC (20% EtOAc in hexane). After completion, the reaction was quenched with water (10 mL) and the mixture was extracted with DCM (2×20 mL). The combined organic layer was dried over anhydrous sodium sulfate and filtered, and the filtrate was concentrated under reduced pressure. The crude was purified by silica gel column chromatography using EtOAc in hexane (product eluted at 3% EtOAc in hexane) to afford 1,3-dibromo-2-(ethoxy methyl) propane (2.1 g, 67.7% yield) as a liquid. ¹HNMR (400 MHz, DMSO-d₆): δ 3.67-3.54 (m, 4H), 3.51-3.40 (m, 4H), 2.33-2.23 (m, 1H), 1.12 (t, J=7.0 Hz, 2H), one proton merged with solvent peaks.

### Step 4. Benzyl ((1r,3r)-3-(3-(ethoxymethyl)azetidin-1-yl)-3-methylcyclobutyl)(isopropyl)carbamate

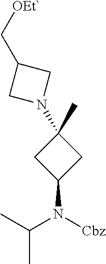

This compound was synthesized following the procedure described for the synthesis of Intermediate 40, step 1, starting with 1 equivalent of benzyl ((1r,3r)-3-amino-3-methylcyclobutyl)(isopropyl)carbamate, except that 1.1 equivalents of 1,3-dibromo-2-(ethoxy methyl) propane was used. After work-up, the crude was purified by reverse phase chromatography using a SiliCycle SiliaSep C18 column (40 g) on Biotage isolera one (Mobile phase A: 10 mM NH₄HCO₃ in water, B: ACN) to obtain benzyl ((1r,3r)-3-(3-(ethoxymethyl)azetidin-1-yl)-3-methylcyclobutyl)(isopropyl)carbamate as a liquid.

Yield: 16.65%; ¹H NMR (400 MHz, DMSO-d₆): δ 7.52-7.28 (m, 5H), 5.08 (s, 2H), 4.10-3.90 (m, 2H), 3.52-3.38 (m, 4H), 3.17 (t, J=7.1 Hz, 2H), 2.86 (t, J=6.3 Hz, 2H), 2.16-2.07 (m, 3H), 1.16-1.06 (m, 9H), 0.97 (br s, 3H), two protons merged with solvent peaks; LCMS (Method A): Rt=2.16 min, m/z=375.2 [M+H]⁺, 84.60%.

### Step 5. (1r,3r)-3-(3-(Ethoxy methyl)azetidin-1-yl)-N-isopropyl-3-methylcyclobutan-1-amine

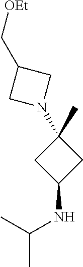 [SMILES: CCOCC1CN([C@]2(C)C[C@@H](NC(C)C)C2)C1]

This compound was synthesized following the procedure described for the synthesis of Intermediate 38, step 2, starting with 1 equivalent of benzyl ((1r,3r)-3-(3-(ethoxy methyl)azetidin-1-yl)-3-methylcyclobutyl)(isopropyl)carbamate, except that 0.352 equivalent of Pd—C was used. After filtration, the filtrate was concentrated under reduced pressure to obtain crude (1r,3r)-3-(3-(ethoxy methyl)azetidin-1-yl)-N-isopropyl-3-methylcyclobutan-1-amine as a gummy liquid. This compound was used without further purification.

Yield: 97%; ¹H NMR (400 MHz, DMSO-d₆): δ 3.49-3.37 (m, 5H), 3.20-3.09 (m, 3H), 2.84 (t, J=6.3 Hz, 2H), 2.77-2.61 (m, 1H), 2.49-2.40 (m, 2H), 2.30-2.21 (m, 2H), 1.10 (t, J=7.0 Hz, 3H), 1.04 (s, 3H), 0.93 (d, J=6.2 Hz, 6H), one proton merged with solvent peaks.

### Step 6. N-((1r,3r)-3-(3-(Ethoxy methyl)azetidin-1-yl)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

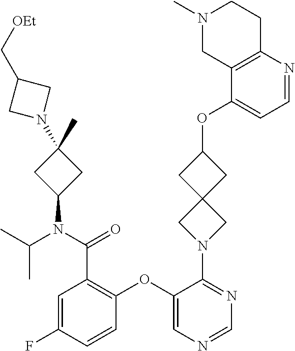 [SMILES: CCOCC1CN([C@]2(C)C[C@@H](N(C(=O)c3cc(F)ccc3Oc3cncnc3N3CC4(CC(Oc5ccnc6c5CN(C)CC6)C4)C3)C(C)C)C2)C1]

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.3 equivalents of (1r,3r)-3-(3-(ethoxymethyl)azetidin-1-yl)-N-isopropyl-3-methylcyclobutan-1-amine. After work-up, the crude was purified by reverse phase chromatography using a SiliCycle SiliaSep C18 column (80 g) on Biotage isolera one (Mobile phase A: 10 mM NH₄HCO₃ in water, B: ACN) to obtain N-((1r,3r)-3-(3-(ethoxymethyl)azetidin-1-yl)-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 17.58%; ¹H NMR (400 MHz, DMSO-d₆): δ 8.32-8.24 (m, 1H), 8.18 (d, J=5.5 Hz, 1H), 7.79-7.59 (m, 1H), 7.34-7.04 (m, 3H), 6.71-6.60 (m, 1H), 4.75 (quin, J=6.4 Hz, 1H), 4.39-4.08 (m, 5H), 3.78-3.61 (m, 2H), 3.51-3.41 (m, 3H), 3.39-3.35 (m, 3H), 3.24-2.98 (m, 2H), 2.93-2.86 (m, 1H), 2.84-2.74 (m, 5H), 2.63 (br t, J=5.7 Hz, 3H), 2.36 (s, 3H), 2.30-1.95 (m, 4H), 1.87-1.73 (m, 1H), 1.48-0.90 (m, 12H); LCMS (Method A): Rt=1.77 min, m/z=714.3 [M+H]⁺; HPLC (Method C): Rt=9.28 min, 97.25%; Chiral SFC (Method AAC): Rt=1.26 min, 100%.

### Example 197. 5-Fluoro-N-isopropyl-N-((1r,3r)-3-(3-(methoxymethyl)azetidin-1-yl)-3-methylcyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 197)

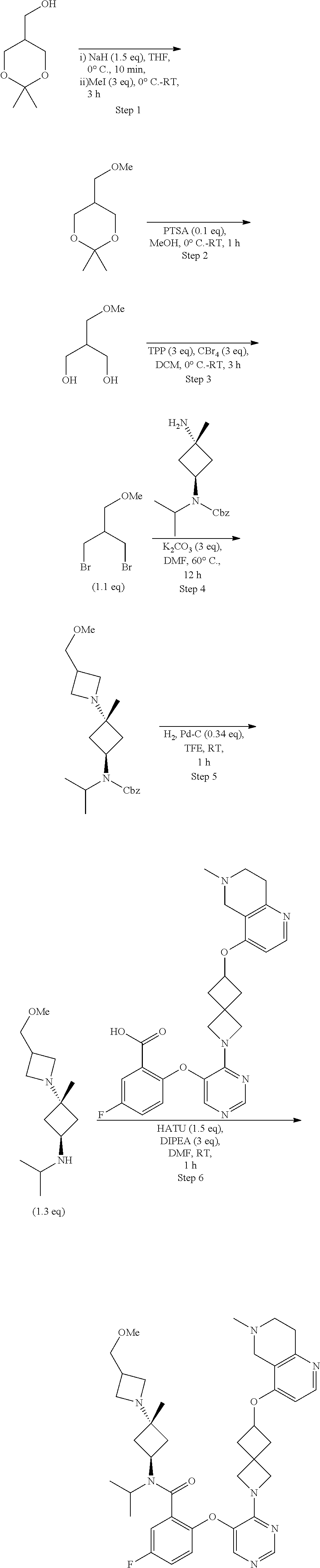

### Step 1. 5-(Methoxymethyl)-2,2-dimethyl-1,3-dioxane

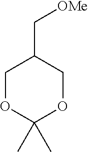

This compound was synthesized following the procedure described for the synthesis of Example 196, step 1, starting with 1 equivalent of (2,2-dimethyl-1,3-dioxan-5-yl)methanol, except that 3 equivalents of methyl iodide was used. After work-up, the crude was purified by silica gel column chromatography using EtOAc in hexane (product eluted at 20% EtOAc in hexane) to afford 5-(methoxymethyl)-2,2-dimethyl-1,3-dioxane as a liquid.

Yield: 63.9%; ¹H NMR (400 MHz, DMSO-d₆): δ 3.89-3.80 (m, 2H), 3.65-3.56 (m, 2H), 3.32 (d, J=6.9 Hz, 2H), 3.23 (s, 3H), 1.93-1.79 (m, 1H), 1.31 (s, 6H).

### Step 2. 2-(Methoxymethyl)propane-1,3-diol

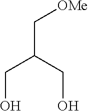

This compound was synthesized following the procedure described for the synthesis of Example 196, step 2, starting with 1 equivalent of 5-(methoxymethyl)-2,2-dimethyl-1,3-dioxane. After work-up, the crude was purified by silica gel column chromatography using MeOH in DCM (product eluted at 5% MeOH in DCM) to obtain 2-(methoxymethyl)propane-1,3-diol as a liquid.

Yield: 76%; ¹H NMR (400 MHz, DMSO-d₆): δ 4.34 (t, J=5.2 Hz, 2H), 3.40 (dt, 1=1.8, 5.5 Hz, 4H), 3.30 (d, J=6.1 Hz, 2H), 3.21 (s, 3H), 1.74 (spt, J=5.9 Hz, 1H).

### Step 3. 1,3-Dibromo-2-(methoxymethyl)propane

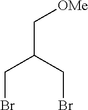

This compound was synthesized following the procedure described for the synthesis of Example 196, step 3, starting with 1 equivalent of 2-(methoxymethyl)propane-1,3-diol. After work-up, the crude was purified by silica gel column chromatography using EtOAc in hexane (product eluted at 5% EtOAc in hexane) to obtain 1,3-dibromo-2-(methoxymethyl)propane as a liquid.

Yield: 77%; ¹H NMR (400 MHz, DMSO-d₆): δ 3.68-3.52 (m, 4H), 3.41 (d, J=6.3 Hz, 2H), 3.28 (s, 3H), 2.31 (spt, J=5.8 Hz, 1H).

### Step 4. Benzyl isopropyl((1r,3r)-3-(3-(methoxymethyl)azetidin-1-yl)-3-methylcyclobutyl)carbamate

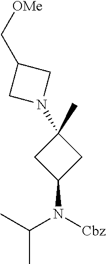 [SMILES: COCC1CN([C@]2(C)C[C@@H](N(C(=O)OCc3ccccc3)C(C)C)C2)C1]

This compound was synthesized following the procedure described for the synthesis of Intermediate 40, step 2, starting with 1 equivalent of benzyl ((1r,3r)-3-amino-3-methylcyclobutyl)(isopropyl)carbamate, except that 1.3 equivalents of 1,3-dibromo-2-(methoxymethyl)propane was used, and DMF was used as the solvent. After work-up, the crude was purified by reverse phase chromatography using a SiliCycle SiliaSep C18 column (40 g) on Biotage isolera one (Mobile phase A: 10 mM NH₄HCO₃ in water, B: ACN) to obtain benzyl isopropyl((1r,3r)-3-(3-(methoxymethyl)azetidin-1-yl)-3-methylcyclobutyl)carbamate as a liquid.

Yield: 4.67%; ¹H NMR (400 MHz, DMSO-d₆): δ 7.47-7.26 (m, 5H), 5.08 (s, 2H), 4.08-3.93 (m, 2H), 3.43 (d, J=6.8 Hz, 2H), 3.24 (s, 3H), 3.17 (t, J=7.1 Hz, 2H), 2.85 (t, J=6.3 Hz, 2H), 2.15-2.06 (m, 4H), 1.12 (d, J=6.8 Hz, 6H), 0.97 (br s, 3H), one proton merged with solvent peaks; LCMS (Method A): Rt=2.01 min, m/z=361.2 [M+H]⁺, 60.91%.

### Step 5. (1r,3r)—N-Isopropyl-3-(3-(methoxymethyl)azetidin-1-yl)-3-methylcyclobutan-1-amine

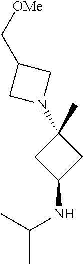 [SMILES: COCC1CN([C@]2(C)C[C@@H](NC(C)C)C2)C1]

This compound was synthesized following the procedure described for the synthesis of Intermediate 38, step 2, starting with 1 equivalent of benzyl isopropyl((1r,3r)-3-(3-(methoxymethyl)azetidin-1-yl)-3-methylcyclobutyl)carbamate, except that 0.34 equivalent of Pd—C was used. After filtration, the filtrate was concentrated under reduced pressure. The residue was reconcentrated from toluene to obtain crude (1r,3r)—N-isopropyl-3-(3-(methoxymethyl)azetidin-1-yl)-3-methylcyclobutan-1-amine as a liquid. This compound was used without further purification.

Yield: 96%; LCMS-ELSD (Method A): Rt=1.42 min, m/z=227.2 [M+H]⁺, 80.30%.

### Step 6. 5-Fluoro-N-isopropyl-N-((1r,3r)-3-(3-((methoxymethyl)azetidin-1-yl)-3-methylcyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

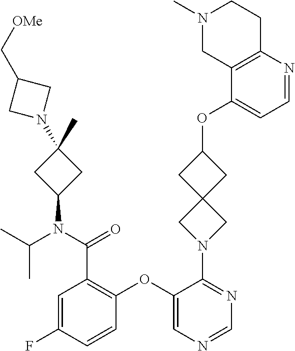 [SMILES: COCC1CN([C@]2(C)C[C@@H](N(C(=O)c3cc(F)ccc3Oc3cncnc3N3CC4(CC(Oc5ccnc6c5CN(C)CC6)C4)C3)C(C)C)C2)C1]

Step 6 was performed following the same procedure in two batches.

This compound was synthesized following the procedure described for the synthesis of Example 1, Step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.3 equivalents of (1r,3r)—N-isopropyl-3-(3-(methoxymethyl)azetidin-1-yl)-3-methylcyclobutan-1-amine. After work-up, the crude from both batches were mixed and purified by reverse phase chromatography using a SiliCycle SiliaSep C18 column (80 g) on Biotage isolera one (Mobile phase A: 10 mM NH₄HCO₃ in water, B: ACN) to obtain 5-fluoro-N-isopropyl-N-((1r,3r)-3-(3-(methoxymethyl)azetidin-1-yl)-3-methylcyclobutyl)-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Combined yield: 7.3%; ¹H NMR (400 MHz, DMSO-d₆): δ 8.29-8.25 (m, 1H), 8.18 (d, J=5.5 Hz, 1H), 7.79-7.60 (m, 1H), 7.35-6.99 (m, 3H), 6.70-6.62 (m, 1H), 4.75 (quin, J=6.7 Hz, 1H), 4.45-4.05 (m, 4H), 3.85-3.57 (m, 2H), 3.48-3.42 (m, 1H), 3.38-3.37 (m, 2H), 3.27-3.17 (m, 5H), 3.06-2.88 (m, 2H), 2.84-2.74 (m, 5H), 2.63 (br t, J=5.7 Hz, 2H), 2.36 (s, 3H), 2.30-2.21 (m, 2H), 2.17-1.93 (m, 2H), 1.86-1.73 (m, 1H), 1.53-0.90 (m, 9H), two protons merged with solvent peaks; LCMS (Method A): Rt=1.71 min, m/z=700.2 [M+H]⁺; HPLC (Method C): Rt=8.69 min, 97.30%; Chiral SFC (Method AB): Rt=1.36 min, 100%.

### Example 198. N-((1r,3r)-3-(2-Azaspiro[3.3]heptan-2-yl)cyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 198)

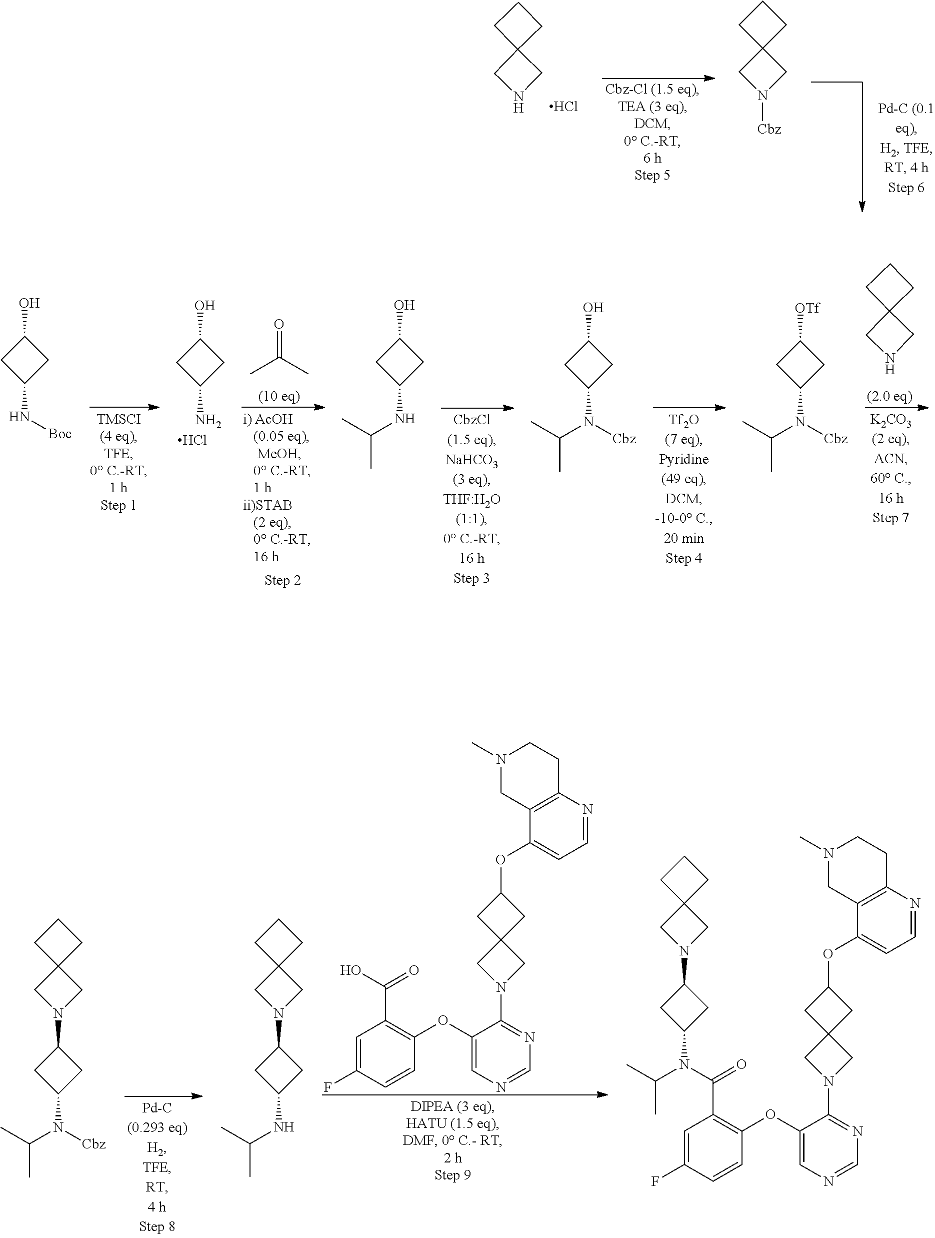

### Step 1. (1s,3s)-3-Aminocyclobutan-1-ol Hydrochloride

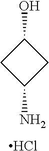

In a 100 mL round bottom flask under a nitrogen atmosphere, tert-butyl ((1s,3s)-3-hydroxycyclobutyl)carbamate (10.0 g, 53.4 mmol) was dissolved in TFE (100 mL). TMSCl (27.3 mL, 214 mmol) was added at 0° C., and the reaction was stirred at RT for 1 h. The reaction progress was monitored by TLC (50% EtOAc in hexane). After completion, the reaction was concentrated under pressure to obtain crude (1s,3s)-3-aminocyclobutan-1-ol hydrochloride (6.6 g, quantitative yield) as a solid. The crude was used without further purification. ¹H NMR (400 MHz, DMSO-d₆): δ 8.31 (br s, 3H), 5.48-5.26 (m, 1H), 3.93-3.82 (m, 1H), 3.17-3.06 (m, 1H), 2.49-2.44 (m, 2H), 2.02-1.87 (m, 2H).

### Step 2. (1s,3s)-3-(Isopropylamino)cyclobutan-1-ol

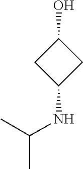

In a dried, 100 mL two neck round bottom flask under a nitrogen atmosphere, (1s,3s)-3-aminocyclobutan-1-ol hydrochloride (6.6 g, 53.4 mmol) was dissolved in MeOH (90 mL). To this solution, acetone (39.2 mL, 534 mmol) and AcOH (0.153 mL, 2.67 mmol) were added at 0° C., and the reaction was stirred at RT for 1 h. Sodium triacetoxyborohydride (22.64 g, 107 mmol) was then added at 0° C., and the reaction was stirred at RT for 16 h. The reaction progress was monitored by TLC (15% MeOH in DCM) and ¹HNMR. After completion, the reaction was filtered through a Celite® pad, and the pad was washed with EtOAc. The filtrate was concentrated under reduced pressure to obtain crude (1s,3s)-3-(isopropylamino)cyclobutan-1-ol (12.0 g, quantitative yield) as a gummy solid. The crude was used without further purification. ¹H NMR (400 MHz, DMSO-d₆): δ 3.80 (quin, J=7.3 Hz, 1H), 2.97-2.80 (m, 2H), 2.49-2.41 (m, 2H), 1.80-1.70 (m, 2H), 1.03 (d, J=6.4 Hz, 6H), two protons merged with solvent peaks; LCMS-ELSD (Method A): Rt=0.483 min, m/z=130.1 [M+H]⁺, 96.86%.

### Step 3. Benzyl ((1s,3s)-3-hydroxycyclobutyl)(isopropyl)carbamate

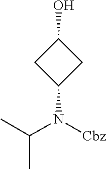 [SMILES: CC(C)N(C(=O)OCc1ccccc1)[C@H]1C[C@@H](O)C1]

In a dried, 250 mL two neck round bottom flask under a nitrogen atmosphere, (1s,3s)-3-(isopropylamino)cyclobutan-1-ol (4 g, 31.0 mmol) was dissolved in THF (20 mL). To this solution, sodium bicarbonate (7.80 g, 93 mmol) and Cbz-Cl (6.66 mL, 46.4 mmol) were added at 0° C., and the reaction was stirred at RT for 16 h. The reaction progress was monitored by TLC (40% EtOAc in hexane). After completion, the reaction was cooled to 0° C.-5° C. and quenched with water (20 mL), and the mixture was extracted with EtOAc (3×50 mL). The combined organic layer was washed with brine solution (50 mL), dried over anhydrous sodium sulfate and filtered, and the filtrate was concentrated under reduced pressure. The crude was purified by silica gel flash column chromatography using EtOAc in hexane (product eluted at 11% EtOAc in hexane) to obtain benzyl ((1s,3s)-3-hydroxycyclobutyl)(isopropyl)carbamate (2.3 g, 26.6% yield) as a liquid.

¹H NMR (400 MHz, DMSO-d₆): δ 7.40-7.28 (m, 5H), 5.07 (s, 2H), 5.00 (d, J=6.8 Hz, 1H), 3.93-3.82 (m, 1H), 3.82-3.70 (m, 2H), 2.41-2.33 (m, 2H), 2.19-2.04 (m, 2H), 1.18 (d, J=6.9 Hz, 6H).

### Step 4. (1s,3s)-3-(((Benzyloxy)carbonyl)(isopropyl)amino)cyclobutyl Trifluoromethanesulfonate

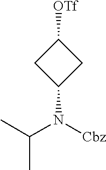 [SMILES: CC(C)N(C(=O)OCc1ccccc1)[C@H]1C[C@@H](OS(=O)(=O)C(F)(F)F)C1]

In a 50 mL round bottom flask under a nitrogen atmosphere, benzyl ((1s,3s)-3-hydroxycyclobutyl(isopropyl)carbamate (0.3 g, 1.139 mmol) was dissolved in DCM (60 mL). To this solution, pyridine (4.51 mL, 55.8 mmol) and trifluoromethanesulfonic anhydride (1.342 mL, 7.97 mmol) were added at −10° C. The reaction was stirred at 0° C. for 20 min. After completion, the reaction was washed with a cold solution of NaHCO₃ (4×50 mL), and then with Na₂CO₃ solution (3×50 mL). The combined organic layer was washed with a cold solution of 1.5N HCl. The combined organic layer was dried over anhydrous sodium sulfate and filtered, and the filtrate was concentrated on reduced pressure to afford crude (1s,3s)-3-(((benzyloxy)carbonyl)(isopropyl)amino)cyclobutyl trifluoromethanesulfonate (360 mg, 80% yield) as a sticky liquid. This material was used without further purification. ¹H NMR (400 MHz, DMSO-d₆): δ 7.44-7.29 (m, 5H), 5.14-5.09 (m, 2H), 4.38-4.24 (m, 1H), 4.18-3.99 (m, 1H), 2.92-2.77 (m, 2H), 2.48-2.43 (m, 2H), 1.13-1.08 (m, 6H), one proton merged with solvent peaks.

### Step 5. Benzyl 2-azaspiro[3.3]heptane-2-carboxylate

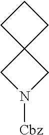 [SMILES: O=C(OCc1ccccc1)N1CC2(CCC2)C1]

To a stirred solution of 2-azaspiro[3.3]heptane hydrochloride (500 mg, 3.74 mmol) and triethylamine (2.62 mL, 18.71 mmol) in DCM (8 mL), Cbz-Cl (0.801 mL, 5.61 mmol) was added at 0° C. The reaction was stirred at RT for 6 h. The reaction progress was monitored by LCMS and TLC (50% EtOAc in hexane). The reaction was quenched with water (5 mL) and the mixture was extracted with DCM (3×10 mL). The combined organic layer was dried over anhydrous sodium sulfate and filtered, and the filtrate was concentrated under reduced pressure. The crude product was purified by silica gel flash column chromatography using EtOAc in hexane (50% EtOAc in hexane) to obtain benzyl 2-azaspiro[3.3]heptane-2-carboxylate (530 mg, 53.2% yield) as a sticky liquid. ¹H NMR (400 MHz, DMSO-d₆): δ 7.40-7.28 (m, 5H), 5.01 (s, 2H), 3.89 (br s, 4H), 2.11 (t, J=7.6 Hz, 4H), 1.75 (quin, J=7.6 Hz, 2H); LCMS (Method A): Rt=2.03 min, m/z=232.1 [M+H]⁺, 86.90%.

### Step 6. 2-Azaspiro[3.3]heptane

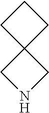

This compound was synthesized following the procedure described for the synthesis of Example 38, step 2, starting with 1 equivalent of benzyl 2-azaspiro[3.3]heptane-2-carboxylate, except that 0.1 equivalent of Pd—C was used. After filtration, the filtrate was concentrated under reduced pressure to afford crude 2-azaspiro[3.3]heptane as a liquid. This material was used without further purification.

Yield: Quantitative; ¹H NMR (400 MHz, DMSO-d₆): δ 3.08-2.97 (m, 4H), 2.00 (t, J=7.5 Hz, 4H), 1.81-1.68 (m, 2H), one proton merged with solvent peaks.

### Step 7. Benzyl ((1r,3r)-3-(2-azaspiro[3.3]heptan-2-yl)cyclobutyl)(isopropyl)carbamate

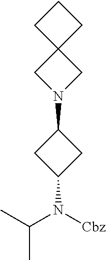 [SMILES: CC(C)N(C(=O)OCc1ccccc1)[C@H]1C[C@H](N2CC3(CCC3)C2)C1]

This compound was synthesized following the procedure described for the synthesis of Example 101, starting with 1 equivalent of (1s,3s)-3-(((benzyloxy)carbonyl)(isopropyl)amino)cyclobutyl trifluoromethanesulfonate and 2 equivalents of 2-azaspiro[3.3]heptane, except that 2 equivalents of K₂CO₃ was used, and ACN was used as the solvent. The reaction was stirred at 60° C. After completion, the reaction was diluted with water and the mixture was extracted with EtOAc. The combined organic layer was washed with brine, dried over anhydrous sodium sulfate and filtered, and the filtrate was concentrated under reduced pressure. The crude material was purified by silica gel flash column chromatography using EtOAc in hexane (product was eluted at 100% EtOAc in hexane) to afford benzyl ((1r,3r)-3-(2-azaspiro[3.3]heptan-2-yl)cyclobutyl)(isopropyl)carbamate as a gummy liquid.

Yield: 43.7%; LCMS (Method A): Rt=2.17 min, m/z=343.0 [M+H]⁺, 62.44%.

### Step 8. (1r,3r)—N-Isopropyl-3-(2-azaspiro[3.3]heptan-2-yl)cyclobutan-1-amine

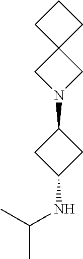

This compound was synthesized following the procedure described for the synthesis of Example 38, step 2, starting with 1 equivalent of benzyl ((1r,3r)-3-(2-azaspiro[3.3]heptan-2-yl)cyclobutyl)(isopropyl)carbamate, except that 0.293 equivalent of Pd—C was used. After filtration, the filtrate was concentrated under reduced pressure to afford crude (1r,3r)—N-isopropyl-3-(2-azaspiro[3.3]heptan-2-yl)cyclobutan-1-amine as a liquid. This material was used without further purification.

Yield: 97%; ¹H NMR (400 MHz, DMSO-d₆): δ 3.88-3.74 (m, 1H), 3.22-3.08 (m, 2H), 2.24-1.96 (m, 10H), 1.82-1.73 (m, 4H), 1.15 (d, J=6.5 Hz, 6H), one proton merged with solvent peaks.

### Step 9. N-((1r,3r)-3-(2-Azaspiro[3.3]heptan-2-yl)cyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

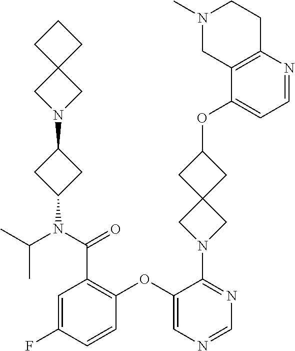 [SMILES: CC(C)N(C(=O)c1cc(F)ccc1Oc1cncnc1N1CC2(CC(Oc3ccnc4c3CN(C)CC4)C2)C1)[C@H]1C[C@H](N2CC3(CCC3)C2)C1]

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1 equivalent of (1r,3r)—N-isopropyl-3-(2-azaspiro[3.3]heptan-2-yl)cyclobutan-1-amine. After completion, the reaction was concentrated under reduced pressure. The crude compound was purified by reverse phase chromatography using a SiliCycle SiliaSep C18 column (40 g) on Biotage isolera one (Mobile phase A: 10 mM NH₄HCO₃ in water, B: ACN), then by Prep HPLC (Method L) to afford N-((1r,3r)-3-(2-azaspiro[3.3]heptan-2-yl)cyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide as a solid.

Yield: 4.66%; ¹H NMR (400 MHz, DMSO-d₆): δ 8.31-8.25 (m, 1H), 8.18 (d, J=5.6 Hz, 1H), 7.77-7.66 (m, 1H), 7.31-7.04 (m, 3H), 6.66 (d, J=5.5 Hz, 1H), 4.75 (quin, J=6.3 Hz, 1H), 4.34-4.07 (m, 5H), 4.04-3.62 (m, 2H), 3.02-2.87 (m, 5H), 2.86-2.74 (m, 5H), 2.63 (br t, J=5.8 Hz, 2H), 2.37 (s, 3H), 2.29-2.21 (m, 2H), 2.12-1.91 (m, 6H), 1.83-1.64 (m, 4H), 1.45-0.90 (m, 6H); LCMS (Method A): Rt=1.84 min, m/z=682.2 [M+H]⁺; HPLC (Method C): Rt=9.30 min, 98.08%; Chiral SFC (Method Y): Rt=3.74 min, 100%.

### Example 199. N-((1r,3r)-3-(Dimethylamino)-3-methylcyclobutyl)-5-fluoro-N-isopropyl-2-((4-(6-((2-methyl-1,2,3,4-tetrahydroisoquinolin-8-yl)oxy)-2-azaspiro[3.3]heptan-2-yl)pyrimidin-5-yl)oxy)benzamide (Compound No. 199)

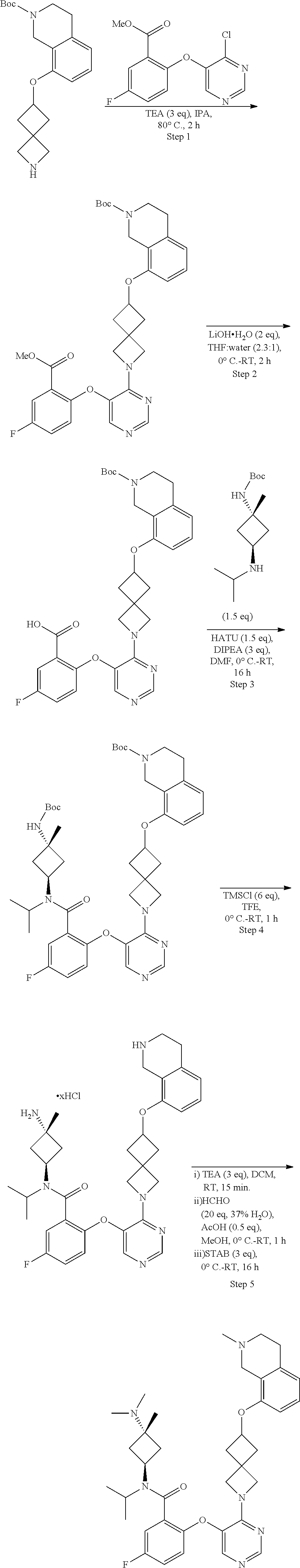

### Step 1. tert-Butyl 8-((2-(5-(4-fluoro-2-(methoxycarbonyl)phenoxy)pyrimidin-4-yl)-2-azaspiro[3.3]heptan-6-yl)oxy)-3,4-dihydroisoquinoline-2(1H)-carboxylate

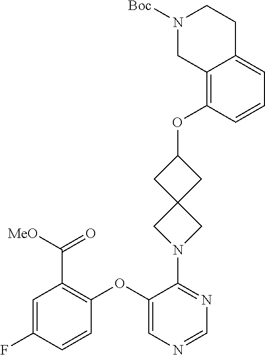 [SMILES: COC(=O)c1cc(F)ccc1Oc1cncnc1N1CC2(CC(Oc3cccc4c3CN(C(=O)OC(C)(C)C)CC4)C2)C1]

This compound was synthesized following the procedure described for the synthesis of Intermediate 24, step 3, starting with 1 equivalent of methyl 2-((4-chloropyrimidin-5-yl)oxy)-5-fluorobenzoate and 1.1 equivalents of tert-butyl 8-((2-azaspiro[3.3]heptan-6-yl)oxy)-3,4-dihydroisoquinoline-2(1H)-carboxylate. After completion, the crude was purified by silica gel flash column chromatography using EtOAc in hexane (product eluted at 80% EtOAc in hexane) to obtain tert-butyl 8-((2-(5-(4-fluoro-2-(methoxycarbonyl)phenoxy)pyrimidin-4-yl)-2-azaspiro[3.3]heptan-6-yl)oxy)-3,4-dihydroisoquinoline-2(1H)-carboxylate as a solid.

Yield: 47%; ¹H NMR (400 MHz, DMSO-d₆): δ 8.32-8.26 (m, 1H), 7.70-7.64 (m, 2H), 7.49 (ddd, J=3.3, 7.9, 9.0 Hz, 1H), 7.13-7.06 (m, 2H), 6.73 (d, J=7.5 Hz, 1H), 6.65 (d, J=8.1 Hz, 1H), 4.67 (quin, J=6.6 Hz, 1H), 4.34 (br s, 2H), 4.27 (s, 2H), 4.20 (s, 2H), 3.81 (s, 3H), 3.52 (br t, J=5.6 Hz, 2H), 2.82-2.69 (m, 4H), 2.29-2.21 (m, 2H), 1.45-1.39 (m, 9H); LCMS (Method B): Rt=2.16 min, m/z=591.2 [M+H]⁺, 79.32%.

### Step 2. 2-((4-(6-((2-(tert-Butoxycarbonyl)-1,2,3,4-tetrahydroisoquinolin-8-yl)oxy)-2-azaspiro[3.3]heptan-2-yl)pyrimidin-5-yl)oxy)-5-fluorobenzoic Acid

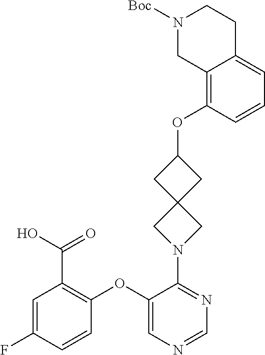 [SMILES: CC(C)(C)OC(=O)N1CCc2cccc(OC3CC4(C3)CN(c3ncncc3Oc3ccc(F)cc3C(=O)O)C4)c2C1]

This compound was synthesized following the procedure described for the synthesis of Intermediate 24, step 4, starting with 1 equivalent of tert-butyl 8-((2-(5-(4-fluoro-2-(methoxycarbonyl)phenoxy)pyrimidin-4-yl)-2-azaspiro[3.3]heptan-6-yl)oxy)-3,4-dihydroisoquinoline-2(1H)-carboxylate, except that 2 equivalents of LiOH·H₂O was used, and THF:H₂O (2.3:1) was used as the solvent. After completion, the reaction was concentrated under reduced pressure. The crude was dissolved in water, acidified with 1.5N HCl (˜pH-5), and then neutralized with solid sodium bicarbonate (˜pH-7). The solid was filtered and dried under vacuum to obtain crude 2-((4-(6-((2-(tert-butoxycarbonyl)-1,2,3,4-tetrahydroisoquinolin-8-yl)oxy)-2-azaspiro[3.3]heptan-2-yl)pyrimidin-5-yl)oxy)-5-fluorobenzoic acid as a solid. This material was used without further purification.

Yield: 60.5%; ¹H NMR (400 MHz, DMSO-d₆): δ 13.50-13.09 (m, 1H), 8.28-8.22 (m, 1H), 7.63-7.54 (m, 2H), 7.42 (br s, 1H), 7.13-7.06 (m, 2H), 6.77-6.71 (m, 1H), 6.66 (d, J=8.2 Hz, 1H), 4.72-4.61 (m, 1H), 4.35 (br s, 2H), 4.30 (br s, 2H), 4.22 (br s, 2H), 3.55-3.50 (m, 2H), 2.82-2.70 (m, 4H), 2.31-2.20 (m, 2H), 1.42 (s, 9H); LCMS (Method A): Rt=1.65 min, m/z=577.3 [M+H]⁺, 85.27%.

### Step 3. tert-Butyl 8-((2-(5-(2-(((1r,3r)-3-((tert-butoxycarbonyl)amino)-3-methylcyclobutyl)(isopropyl)carbamyl)-4-fluorophenoxy)pyrimidin-4-yl)-2-azaspiro[3.3]heptan-6-yl)oxy)-3,4-dihydroisoquinoline-2(1H)-carboxylate

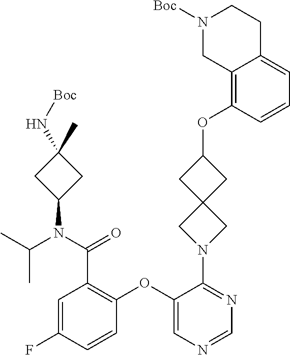 [SMILES: CC(C)N(C(=O)c1cc(F)ccc1Oc1cncnc1N1CC2(CC(Oc3cccc4c3CN(C(=O)OC(C)(C)C)CC4)C2)C1)[C@H]1C[C@](C)(NC(=O)OC(C)(C)C)C1]

This compound was synthesized following the procedure described for the synthesis of Example 1, step 2, starting with 1 equivalent of 2-((4-(6-((2-(tert-butoxycarbonyl)-1,2,3,4-tetrahydroisoquinolin-8-yl)oxy)-2-azaspiro[3.3]heptan-2-yl)pyrimidin-5-yl)oxy)-5-fluorobenzoic acid and 1.5 equivalents of tert-butyl ((1r,3r)-3-(isopropylamino)-1-methylcyclobutyl)carbamate. After work-up, the crude was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure to afford tert-butyl 8-((2-(5-(2-(((1r,3r)-3-((tert-butoxycarbonyl)amino)-3-methylcyclobutyl)(isopropyl)carbamoyl)-4-fluorophenoxy)pyrimidin-4-yl)-2-azaspiro[3.3]heptan-6-yl)oxy)-3,4-dihydroisoquinoline-2(1H)-carboxylate as a solid.

Yield: 70.6%; ¹H NMR (400 MHz, DMSO-d₆): δ 8.30 (s, 1H), 7.74 (br d, J=3.4 Hz, 1H), 7.32-7.16 (m, 2H), 7.20-7.01 (m, 2H), 6.94 (br s, 1H), 6.74 (d, J=7.6 Hz, 1H), 6.65 (br d, J=8.3 Hz, 1H), 4.65 (br s, 1H), 4.36 (br s, 2H), 4.32-4.02 (m, 5H), 3.97-3.63 (m, 2H), 3.52 (br t, J=5.3 Hz, 2H), 3.04-2.92 (m, 1H), 2.87-2.69 (m, 4H), 2.32-2.17 (m, 3H), 2.02-1.92 (m, 1H), 1.44-1.42 (m, 9H), 1.41-1.26 (m, 15H), 1.08-0.90 (m, 3H); LCMS (Method A): Rt=2.63 min, m/z=801.2 [M+H]⁺, 88.27%.

### Step 4. N-((1r,3r)-3-Amino-3-methylcyclobutyl)-5-fluoro-N-isopropyl-2-((4-(6-((1,2,3,4-tetrahydroisoquinolin-8-yl)oxy)-2-azaspiro[3.3]heptan-2-yl)pyrimidin-5-yl)oxy)benzamide xHCl

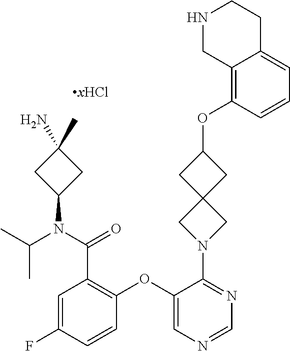 [SMILES: CC(C)N(C(=O)c1cc(F)ccc1Oc1cncnc1N1CC2(CC(Oc3cccc4c3CNCC4)C2)C1)[C@H]1C[C@](C)(N)C1]

This compound was synthesized following the procedure described for the synthesis of Example 20, step 3, starting with 1 equivalent of tert-butyl 8-((2-(5-(2-(((1r,3r)-3-((tert-butoxycarbonyl)amino)-3-methylcyclobutyl)(isopropyl)carbamoyl)-4-fluorophenoxy)pyrimidin-4-yl)-2-azaspiro[3.3]heptan-6-yl)oxy)-3,4-dihydroisoquinoline-2(1H)-carboxylate, except that 6 equivalents of TMSCl was used. After completion, the reaction was concentrated under reduced pressure and the residue was triturated with EtOAc. The organic layer was decanted and the solid was dried under vacuum to obtain crude N-((1r,3r)-3-amino-3-methylcyclobutyl)-5-fluoro-N-isopropyl-2-((4-(6-((1,2,3,4-tetrahydroisoquinolin-8-yl)oxy)-2-azaspiro[3.3]heptan-2-yl)pyrimidin-5-yl)oxy)benzamide xHCl as a solid. This material was used without further purification.

Yield: Quantitative: LCMS (Method C): Rt=1.51 min, m/z=601.3 [M+H]⁺, 90.17%.

### Step 5. N-((1r,3r)-3-(Dimethylamino)-3-methylcyclobutyl)-5-fluoro-N-isopropyl-2-((4-(6-((2-methyl-1,2,3,4-tetrahydroisoquinolin-8-yl)oxy)-2-azaspiro[3.3]heptan-2-yl)pyrimidin-5-yl)oxy)benzamide

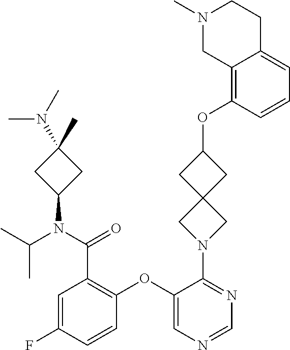 [SMILES: CC(C)N(C(=O)c1cc(F)ccc1Oc1cncnc1N1CC2(CC(Oc3cccc4c3CN(C)CC4)C2)C1)[C@H]1C[C@](C)(N(C)C)C1]

This compound was synthesized following the procedure described for the synthesis of Example 20, step 4, starting with 1 equivalent of N-((1r,3r)-3-amino-3-methylcyclobutyl)-5-fluoro-N-isopropyl-2-((4-(6-((1,2,3,4-tetrahydroisoquinolin-8-yl)oxy)-2-azaspiro[3.3]heptan-2-yl)pyrimidin-5-yl)oxy)benzamide xHCl, except that 3 equivalents of TEA, 20 equivalents of formaldehyde (37% in H₂O), 0.5 equivalent of AcOH, and 3 equivalents of STAB were used. After work-up, the crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford N-((1r,3r)-3-(dimethylamino)-3-methylcyclobutyl)-5-fluoro-N-isopropyl-2-((4-(6-((2-methyl-1,2,3,4-tetrahydroisoquinolin-8-yl)oxy)-2-azaspiro[3.3]heptan-2-yl)pyrimidin-5-yl)oxy)benzamide as a solid.

Yield: 33%; ¹H NMR (400 MHz, DMSO-d₆): δ 8.30-8.25 (m, 1H), 7.78-7.59 (m, 11H), 7.32-7.01 (m, 4H), 6.68 (d, J=7.6 Hz, 1H), 6.56 (br d, J=7.9 Hz, 1H), 4.63 (quin, J=6.6 Hz, 1H), 4.35-4.10 (m, 4H), 4.05-3.77 (m, 1H), 3.75-3.65 (m, 1H), 2.66-2.57 (m, 1H), 2.81-2.71 (m, 4H), 2.34 (s, 4H), 2.26-2.18 (m, 2H), 2.15-1.91 (s, 8H), 1.81-1.68 (m, 1H), 1.47-1.28 (m, 3H), 1.16-0.90 (m, 6H), three protons merged with solvent peaks; LCMS (Method C): Rt=1.57 min, m/z=643.4 [M+H]⁺; HPLC (Method C): Rt=10.25 min, 99.92%; Chiral SFC (Method AS): Rt=3.27 min, 100%.

### Example 200. N-((1r,3r)-3-(Dimethylamino)-3-methylcyclobutyl)-5-fluoro-N-isopropyl-2-((4-(6-((2-methylisoindolin-4-yl)oxy)-2-azaspiro[3.3]heptan-2-yl)pyrimidin-5-yl)oxy)benzamide (Compound No. 200)

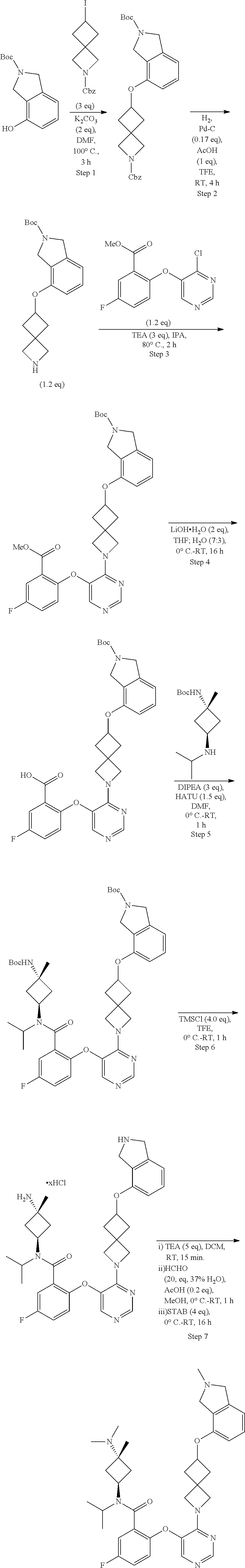

### Step 1. tert-Butyl 4-((2-((benzyloxy)carbonyl)-2-azaspiro[3.3]heptan-6-yl)oxy)isoindoline-2-carboxylate

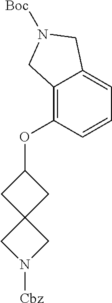 [SMILES: CC(C)(C)OC(=O)N1Cc2cccc(OC3CC4(C3)CN(C(=O)OCc3ccccc3)C4)c2C1]

This compound was synthesized following the procedure described for the synthesis of Intermediate 15, step 3, starting with 1 equivalent of tert-butyl 4-hydroxyisoindoline-2-carboxylate and 3 equivalents of benzyl 6-iodo-2-azaspiro[3.3]heptane-2-carboxylate, except that 2 equivalents of K₂CO₃ was used. After work-up, the crude product was purified by silica gel flash column chromatography using EtOAc in hexane (product eluted at 60% EtOAc in hexane), then by reverse phase chromatography using a SiliCycle SiliaSep C18 column (120 g) on Biotage isolera one (Mobile phase A: 10 mM NH₄HCO₃ in water, B: ACN) to obtain tert-butyl 4-((2-((benzyloxy)carbonyl)-2-azaspiro[3.3]heptan-6-yl)oxy)isoindoline-2-carboxylate as a solid.

Yield: 61.5%; ¹H NMR (400 MHz, DMSO-d₆): δ 7.40-7.29 (m, 5H), 7.23 (t, J=7.9 Hz, 1H), 6.93-6.85 (m, 1H), 6.74-6.69 (m, 1H), 5.03 (s, 2H), 4.68 (quin, J=6.7 Hz, 1H), 4.56 (br d, J=10.5 Hz, 2H), 4.45 (br d, J=10.1 Hz, 2H), 4.06-3.87 (m, 4H), 2.81-2.68 (m, 2H), 2.23 (dt, J=3.7, 6.5 Hz, 2H), 1.46 (s, 9H); LCMS (Method B): Rt=2.58 min, m/z=365.3 [M+H-Boc]⁺, 85.26%.

### Step 2. tert-Butyl 4-((2-azaspiro[3.3]heptan-6-yl)oxy)isoindoline-2-carboxylate

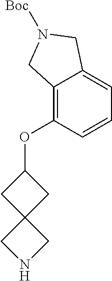 [SMILES: CC(C)(C)OC(=O)N1Cc2cccc(OC3CC4(CNC4)C3)c2C1]

This compound was synthesized following the procedure described for the synthesis of Intermediate 15, step 4, starting with 1 equivalent of tert-butyl 4-((2-((benzyloxy)carbonyl)-2-azaspiro[3.3]heptan-6-yl)oxy)isoindoline-2-carboxylate, except that 1 equivalent of AcOH and 0.17 equivalent of Pd—C were used. After completion, the reaction was filtered through a Celite® pad, and the pad was washed with MeOH. The filtrate was concentrated under reduced pressure to afford crude tert-butyl 4-((2-azaspiro[3.3]heptan-6-yl)oxy)isoindoline-2-carboxylate as a liquid. This material was used without further purification.

Yield: Quantitative; LCMS (Method B): Rt=1.43 min, m/z=331.4 [M+H]⁺, 99.10%.

### Step 3. tert-Butyl 4-((2-(5-(4-fluoro-2-(methoxycarbonyl)phenoxy)pyrimidin-4-yl)-2-azaspiro[3.3]heptan-6-yl)oxy)isoindoline-2-carboxylate

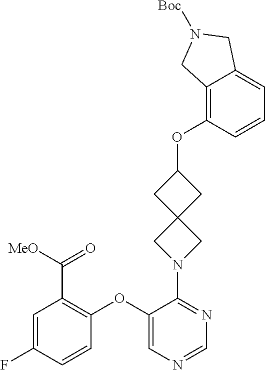 [SMILES: COC(=O)c1cc(F)ccc1Oc1cncnc1N1CC2(CC(Oc3cccc4c3CN(C(=O)OC(C)(C)C)C4)C2)C1]

This compound was synthesized following the procedure described for the synthesis of Intermediate 22, step 1, starting with 1 equivalent of methyl 2-((4-chloropyrimidin-5-yl)oxy)-5-fluorobenzoate and 1.2 equivalents of tert-butyl 4-((2-azaspiro[3.3]heptan-6-yl)oxy)isoindoline-2-carboxylate. After completion, the reaction was concentrated under reduced pressure and diluted with water, and the mixture was extracted with 10% MeOH in DCM. The combined organic layer was dried over anhydrous sodium sulfate and filtered, and the filtrate was concentrated under reduced pressure. The crude product was purified by silica gel flash column chromatography using EtOAc in hexane (product eluted at 80% EtOAc in hexane) to obtain tert-butyl 4-((2-(5-(4-fluoro-2-(methoxycarbonyl)phenoxy)pyrimidin-4-yl)-2-azaspiro[3.3]heptan-6-yl)oxy)isoindoline-2-carboxylate as a solid.

Yield: 69.3%; ¹H NMR (400 MHz, DMSO-d₆): δ 8.32-8.27 (m, 1H), 7.69-7.64 (m, 2H), 7.52-7.45 (m, 1H), 7.22 (t, J=7.8 Hz, 1H), 7.10 (td, J=4.1, 8.8 Hz, 1H), 6.91-6.85 (m, 1H), 6.73 (d, J=8.2 Hz, 1H), 4.71 (quin, J=6.6 Hz, 1H), 4.56 (br d, J=10.8 Hz, 2H), 4.44 (br d, J=11.6 Hz, 2H), 4.27 (br s, 2H), 4.18 (s, 2H), 3.81 (s, 3H), 2.81-2.72 (m, 2H), 2.30-2.23 (m, 2H), 1.46 (s, 9H); LCMS (Method E): Rt=2.81 min, m/z=577.2 [M+H]⁺, 99.22%.

### Step 4. 2-((4-(6-((2-(tert-Butoxycarbonyl)isoindolin-4-yl)oxy)-2-azaspiro[3.3]heptan-2-yl)pyrimidin-5-yl)oxy)-5-fluorobenzoic Acid

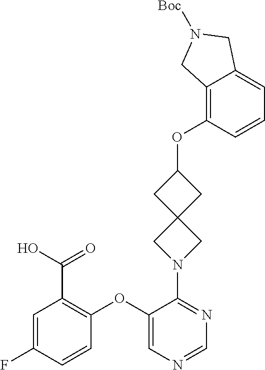 [SMILES: CC(C)(C)OC(=O)N1Cc2cccc(OC3CC4(C3)CN(c3ncncc3Oc3ccc(F)cc3C(=O)O)C4)c2C1]

In a 250 mL round bottom flask, tert-butyl 4-((2-(5-(4-fluoro-2-(methoxycarbonyl)phenoxy)pyrimidin-4-yl)-2-azaspiro[3.3]heptan-6-yl)oxy)isoindoline-2-carboxylate (6.4 g, 11.10 mmol) was dissolved in THF:H₂O (7:3, 28.5 mL). To this solution, LiOH·H₂O (0.931 g, 22.20 mmol) was added at 0° C. The reaction was stirred at RT for 16 h. The reaction progress was monitored by TLC (10% MeOH in DCM). After completion, the reaction was concentrated under reduced pressure. The residue was dissolved in water (10 mL) and the solution was acidified with 1.5N HCl solution at 0° C. The precipitate was filtered and the solid obtained was dried under vacuum to obtain crude 2-((4-(6-((2-(tert-butoxycarbonyl)isoindolin-4-yl)oxy)-2-azaspiro[3.3]heptan-2-yl)pyrimidin-5-yl)oxy)-5-fluorobenzoic acid (5.8 g, 90% yield) as a solid. This material was used without further purification. ¹H NMR (400 MHz, DMSO-d₆): δ 8.29-8.18 (m, 1H), 7.59-7.50 (m, 2H), 7.37 (dt, 0.1=3.2, 8.3 Hz, 1H), 7.27-7.13 (m, 1H), 7.07 (dd, J=4.5, 9.0 Hz, 1H), 6.93-6.83 (m, 1H), 6.73 (d, J=8.1 Hz, 1H), 4.71 (quin, J=6.7 Hz, 1H), 4.56 (br d, J=11.0 Hz, 2H), 4.45 (br d, J=10.4 Hz, 2H), 4.30 (br s, 2H), 4.20 (br s, 2H), 2.76 (br dd, J=6.9, 9.9 Hz, 2H), 2.26 (br dd, J=7.0, 10.1 Hz, 2H), 1.45 (s, 9H), one proton merged with solvent peaks; LCMS (Method A): Rt=1.52 min, m/z=563.2 [M+H]⁺, 97.92%.

### Step 5. tert-Butyl 4-((2-(5-(2-((1r,3r)-3-((tert-butoxycarbonyl)amino)-3-methylcyclobutyl)(isopropyl)carbamoyl)-4-fluorophenoxy)pyrimidin-4-yl)-2-azaspiro[3.3]heptan-6-yl)oxy)isoindoline-2-carboxylate

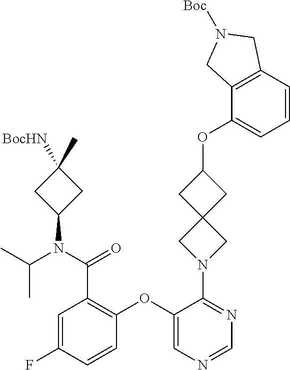 [SMILES: CC(C)N(C(=O)c1cc(F)ccc1Oc1cncnc1N1CC2(CC(Oc3cccc4c3CN(C(=O)OC(C)(C)C)C4)C2)C1)[C@H]1C[C@](C)(NC(=O)OC(C)(C)C)C1]

This compound was synthesized following the procedure described for the synthesis of Example 1, step 2, starting with 1 equivalent of 2-((4-(6-((2-(tert-butoxycarbonyl)isoindolin-4-yl)oxy)-2-azaspiro[3.3]heptan-2-yl)pyrimidin-5-yl)oxy)-5-fluorobenzoic acid and 1 equivalent of tert-butyl ((1r,3r)-3-(isopropylamino)-1-methylcyclobutyl)carbamate. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain tert-butyl 4-((2-(5-(2-(((1r,3r)-3-((tert-butoxycarbonyl)amino)-3-methylcyclobutyl)(isopropyl)carbamoyl)-4-fluorophenoxy)pyrimidin-4-yl)-2-azaspiro[3.3]heptan-6-yl)oxy)isoindoline-2-carboxylate as a solid.

Yield: 74.3%; LCMS (Method A): Rt=2.54 min, m/z=787.2 [M+H]⁺, 98.49%.

### Step 6. N-((1r,3r)-3-Amino-3-methylcyclobutyl)-5-fluoro-2-((4-(6-(isoindolin-4-yloxy)-2-azaspiro[3.3]heptan-2-yl)pyrimidin-5-yl)oxy)-N-isopropylbenzamide xHCl

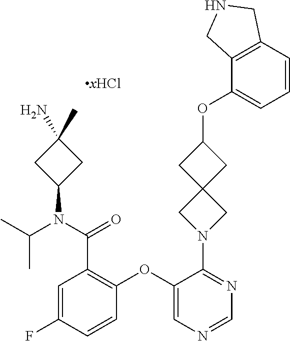 [SMILES: CC(C)N(C(=O)c1cc(F)ccc1Oc1cncnc1N1CC2(CC(Oc3cccc4c3CNC4)C2)C1)[C@H]1C[C@](C)(N)C1]

This compound was synthesized following the procedure described for the synthesis of Example 20, step 3, starting with 1 equivalent of tert-butyl 4-((2-(5-(2-(((1r,3r)-3-((tert-butoxycarbonyl)amino)-3-methylcyclobutyl)(isopropyl)carbamoyl)-4-fluorophenoxy)pyrimidin-4-yl)-2-azaspiro[3.3]heptan-6-yl)oxy)isoindoline-2-carboxylate. After completion, the reaction was concentrated under reduced pressure to obtain crude N-((1r,3r)-3-amino-3-methylcyclobutyl)-5-fluoro-2-((4-(6-(isoindolin-4-yloxy)-2-azaspiro[3.3]heptan-2-yl)pyrimidin-5-yl)oxy)-N-isopropylbenzamide xHCl as a solid. This material was used without further purification.

Yield: Quantitative; LCMS (Method A): Rt=1.82 min, m/z=587.4 [M+H]⁺, 97.14%.

### Step 7. N-((1r,3r)-3-(Dimethylamino)-3-methylcyclobutyl)-5-fluoro-N-isopropyl-2-((4-(6-((2-methylisoindolin-4-yl)oxy)-2-azaspiro[3.3]heptan-2-yl)pyrimidin-5-yl)oxy)benzamide

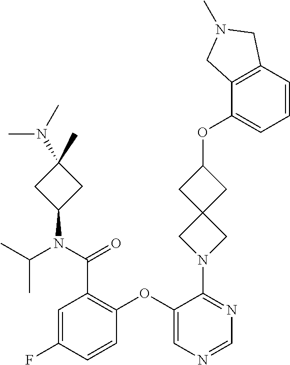 [SMILES: CC(C)N(C(=O)c1cc(F)ccc1Oc1cncnc1N1CC2(CC(Oc3cccc4c3CN(C)C4)C2)C1)[C@H]1C[C@](C)(N(C)C)C1]

This compound was synthesized following the procedure described for the synthesis of Example 20, step 4, starting with 1 equivalent of N-((1r,3r)-3-amino-3-methylcyclobutyl)-5-fluoro-2-((4-(6-(isoindolin-4-yloxy)-2-azaspiro[3.3]heptan-2-yl)pyrimidin-5-yl)oxy)-N-isopropylbenzamide xHCl, except that 5 equivalents of TEA, 20 equivalents of formaldehyde (37% in H₂O), 0.2 equivalent of AcOH and 4 equivalents of STAB were used. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain N-((1r,3r)-3-(dimethylamino)-3-methylcyclobutyl)-5-fluoro-N-isopropyl-2-((4-(6-((2-methylisoindolin-4-yl)oxy)-2-azaspiro[3.3]heptan-2-yl)pyrimidin-5-yl)oxy)benzamide as a solid.

Yield: 28.2%; ¹H NMR (400 MHz, DMSO-d₆): δ 8.32-8.22 (m, 1H), 7.82-7.56 (m, 1H), 7.31-7.06 (m, 4H), 6.80 (d, J=7.4 Hz, 1H), 6.62 (br d, J=7.9 Hz, 1H), 4.65 (quin, J=6.5 Hz, 1H), 4.33-4.09 (m, 4H), 4.05-3.91 (m, 1H), 3.86-3.64 (m, 5H), 2.82-2.71 (m, 2H), 2.45 (s, 3H), 2.29-2.17 (m, 2H), 2.16-1.89 (m, 9H), 1.81-1.69 (m, 1H), 1.48-1.26 (m, 3H), 1.17-0.91 (m, 6H); LCMS (Method C): Rt=1.55 min, m/z=629.5 [M+H]⁺; HPLC (Method C): Rt=9.92 min, 99.66%; Chiral SFC (Method J): Rt=5.72 min, 97.55%.

### Example 201. N-((1r,3r)-3-(Dimethylamino)-3-methylcyclobutyl)-5-fluoro-N-isopropyl-2-((4-(6-((7-methyl-5,6,7,8-tetrahydro-1,7-naphthyridin-4-yl)oxy)-2-azaspiro[3.3]heptan-2-yl)pyrimidin-5-yl)oxy)benzamide (Compound No. 201)

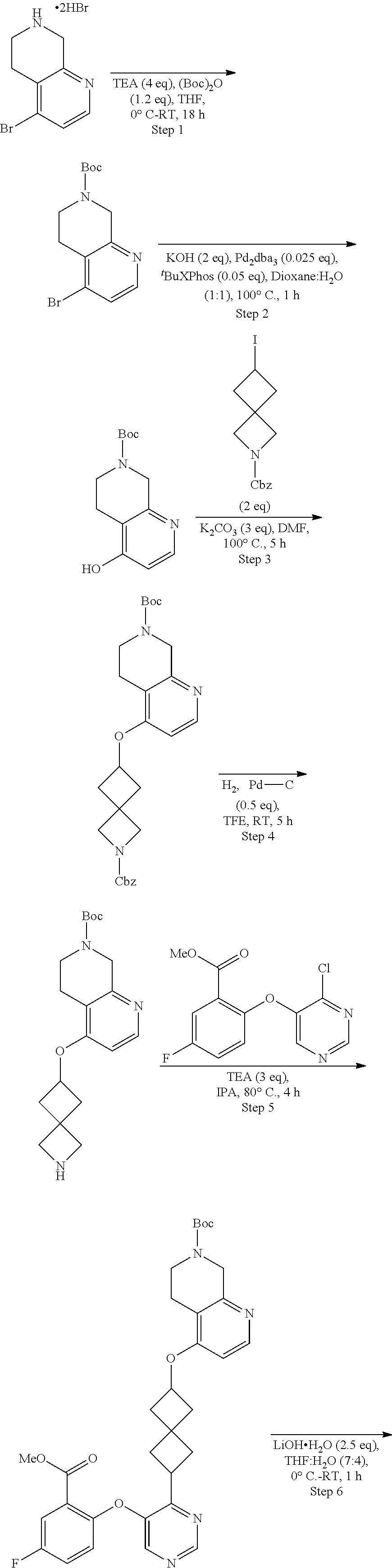

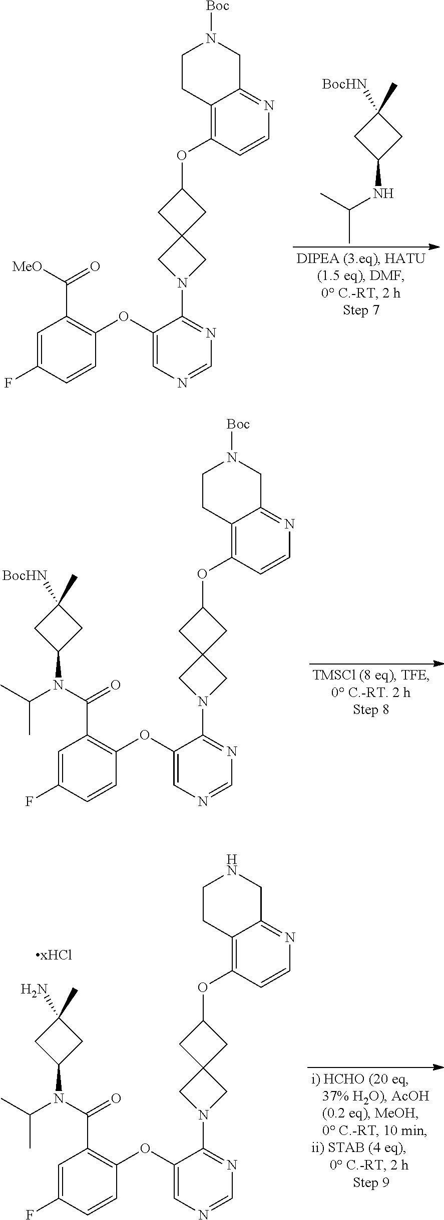 [SMILES: COC(=O)c1cc(F)ccc1Oc1cncnc1N1CC2(CC(Oc3ccnc4c3CCN(C(=O)OC(C)(C)C)C4)C2)C1 ; CC(C)N[C@H]1C[C@](C)(NC(=O)OC(C)(C)C)C1 ; CC(C)N(C(=O)c1cc(F)ccc1Oc1cncnc1N1CC2(CC(Oc3ccnc4c3CCN(C(=O)OC(C)(C)C)C4)C2)C1)[C@H]1C[C@](C)(NC(=O)OC(C)(C)C)C1 ; CC(C)N(C(=O)c1cc(F)ccc1Oc1cncnc1N1CC2(CC(Oc3ccnc4c3CCNC4)C2)C1)[C@H]1C[C@](C)(N)C1]

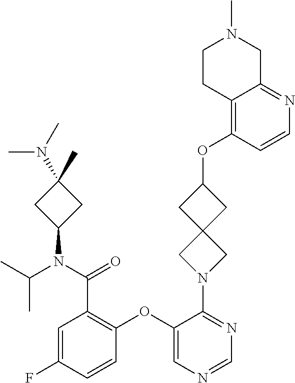 [SMILES: CC(C)N(C(=O)c1cc(F)ccc1Oc1cncnc1N1CC2(CC(Oc3ccnc4c3CCN(C)C4)C2)C1)[C@H]1C[C@](C)(N(C)C)C1]

### Step 1. tert-Butyl 4-bromo-5,8-dihydro-1,7-naphthyridine-7(6H)-carboxylate

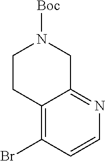 [SMILES: CC(C)(C)OC(=O)N1CCc2c(Br)ccnc2C1]

To a stirred solution of 4-bromo-5,6,7,8-tetrahydro-1,7-naphthyridine dihydrobromide (1 g, 2.67 mmol) in THF (10 mL), TEA (1.487 mL, 10.67 mmol) was added at RT. To this reaction mixture, Boc-anhydride (0.735 mL, 3.20 mmol) was added at 0° C., and the reaction was stirred at RT for 18 h. The reaction progress was monitored by TLC (5% MeOH in DCM). After completion, the reaction was diluted with water (50 mL) and the mixture was extracted with DCM (3×50 mL). The combined organic layer was washed with brine (50 mL), dried over anhydrous sodium sulfate and filtered, and the filtrate was concentrated under reduced pressure. The crude product was purified by silica gel flash column chromatography using EtOAc in hexane (product eluted at 11% EtOAc in hexane) to obtain tert-butyl 4-bromo-5,8-dihydro-1,7-naphthyridine-7(6H)-carboxylate (0.830 g, 98% yield) as a solid. ¹H NMR (400 MHz, DMSO-d₆): δ 8.26 (d, J=5.3 Hz, 1H), 7.60 (d, J=5.3 Hz, 1H), 4.55 (s, 2H), 3.65 (t, J=5.9 Hz, 2H), 2.77 (t, J=5.9 Hz, 2H), 1.43 (s, 9H); LCMS (Method H): Rt=1.73 min, m/z=313.0 [M+H]⁺, 98.99%.

### Step 2. tert-Butyl 4-hydroxy-5,8-dihydro-1,7-naphthyridine-7(6H)-carboxylate

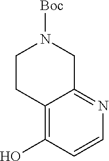 [SMILES: CC(C)(C)OC(=O)N1CCc2c(O)ccnc2C1]

To a stirred solution of tert-butyl 4-bromo-5,8-dihydro-1,7-naphthyridine-7(6H)-carboxylate (800 mg, 2.55 mmol) in 1,4-dioxane:H₂O (1:1, 14 mL), KOH (287 mg, 5.11 mmol) was added at RT. The reaction mixture was purged with a nitrogen gas for 5 min. To this reaction mixture, t-BuXPhos (54.2 mg, 0.128 mmol) and Pd₂(dba)₃ (58.5 mg, 0.064 mmol) were added at RT. The reaction was stirred at 100° C. for 1 h. The reaction progress was monitored by TLC (10% MeOH in DCM). After completion, the reaction was quenched with aqueous NH₄Cl solution (10 mL), and the mixture was extracted with EtOAc (3×25 mL). The combined organic layer was dried over anhydrous sodium sulfate and filtered, and the filtrate was concentrated under reduced pressure. The crude compound was triturated with MTBE (10 mL), and then with hexane (10 mL). The organic layer was decanted, and the residue was dried under vacuum to afford crude tert-butyl 4-hydroxy-5,8-dihydro-1,7-naphthyridine-7(6H)-carboxylate (350 mg, 72.5% yield) as a solid. This material was used without further purification. ¹H NMR (400 MHz, DMSO-d₆): δ 11.55-11.18 (m, 1H), 7.56 (br d, J=6.8 Hz, 1H), 5.97 (br d, J=7.1 Hz, 1H), 4.47-4.22 (m, 2H), 3.50 (br s, 2H), 2.34 (br s, 2H), 1.43 (s, 9H); LCMS (Method H): Rt=1.28 min, m/z=251.1 [M+H]⁺, 99.40%.

### Step 3. tert-Butyl 4-((2-((benzyloxy)carbonyl)-2-azaspiro[3.3]heptan-6-yl)oxy)-5,8-dihydro-1,7-naphthyridine-7(6H)-carboxylate

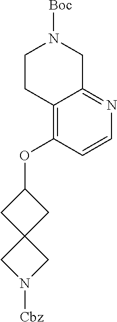 [SMILES: CC(C)(C)OC(=O)N1CCc2c(OC3CC4(C3)CN(C(=O)OCc3ccccc3)C4)ccnc2C1]

This compound was synthesized following the procedure described for the synthesis of Intermediate 24, step 1, starting with 1 equivalent of tert-butyl 4-hydroxy-5,8-dihydro-1,7-naphthyridine-7(6H)-carboxylate and 2 equivalents of benzyl 6-iodo-2-azaspiro[3.3]heptane-2-carboxylate. After work-up, the crude product was purified by silica gel flash column chromatography using EtOAc in hexane (product eluted at 100% EtOAc in hexane) to obtain tert-butyl 4-((2-((benzyloxy)carbonyl)-2-azaspiro[3.3]heptan-6-yl)oxy)-5,8-dihydro-1,7-naphthyridine-7(6H)-carboxylate as a solid.

Yield: 72%; ¹H NMR (400 MHz, DMSO-d₆): δ 8.23 (d, J=5.6 Hz, 1H), 7.42-7.30 (m, 5H), 6.71 (d, J=5.6 Hz, 1H), 5.02 (s, 2H), 4.73 (quin, J=6.6 Hz, 1H), 4.45 (br s, 2H), 4.08-3.89 (m, 4H), 3.56 (br t, J=5.4 Hz, 2H), 2.81-2.73 (m, 2H), 2.60 (br t, J=5.7 Hz, 2H), 2.30-2.21 (m, 2H), 1.42 (s, 9H); LCMS (Method H): Rt=1.81 min, m/z=480.2 [M+H]⁺, 98.49%.

### Step 4. tert-Butyl 4-((2-azaspiro[3.3]heptan-6-yl)oxy)-5,8-dihydro-1,7-naphthyridine-7(6H)-carboxylate

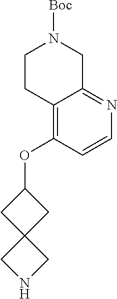 [SMILES: CC(C)(C)OC(=O)N1CCc2c(OC3CC4(CNC4)C3)ccnc2C1]

This compound was synthesized following the procedure described for the synthesis of Intermediate 24, step 2, starting with 1 equivalent of tert-butyl 4-((2-((benzyloxy)carbonyl)-2-azaspiro[3.3]heptan-6-yl)oxy)-5,8-dihydro-1,7-naphthyridine-7(6H)-carboxylate, except that 0.5 equivalent of Pd—C was used. After completion, the reaction was diluted with EtOAc, the mixture was filtered through a Celite® pad, and the filter pad was washed with 10% MeOH in DCM. The filtrate was concentrated under reduced pressure to obtain crude tert-butyl 4-((2-azaspiro[3.3]heptan-6-yl)oxy)-5,8-dihydro-1,7-naphthyridine-7(6H)-carboxylate as a sticky liquid. This material was used without further purification.

Yield: 89%; LCMS (Method B): Rt=0.95 min, m/z=346.2 [M+H]⁺, 96.14%.

### Step 5. tert-Butyl 4-((2-(5-(4-fluoro-2-(methoxycarbonyl)phenoxy)pyrimidin-4-yl)-2-azaspiro[3.3]heptan-6-yl)oxy)-5,8-dihydro-1,7-naphthyridine-7(6H)-carboxylate

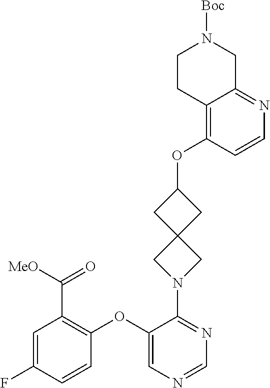 [SMILES: COC(=O)c1cc(F)ccc1Oc1cncnc1N1CC2(CC(Oc3ccnc4c3CCN(C(=O)OC(C)(C)C)C4)C2)C1]

This compound was synthesized following the procedure described for the synthesis of Intermediate 24, step 3, starting with 1 equivalent of methyl 2-((4-chloropyrimidin-5-yl)oxy)-5-fluorobenzoate and 1 equivalent of tert-butyl 4-((2-azaspiro[3.3]heptan-6-yl)oxy)-5,8-dihydro-1,7-naphthyridine-7(6H)-carboxylate. After completion, the reaction was concentrated under reduced pressure. The crude product was purified by silica gel flash column chromatography using EtOAc in hexane, then MeOH in DCM (product eluted at 20% MeOH in DCM). The pure fractions were concentrated under reduced pressure to obtain tert-butyl 4-((2-(5-(4-fluoro-2-(methoxycarbonyl)phenoxy)pyrimidin-4-yl)-2-azaspiro[3.3]heptan-6-yl)oxy)-5,8-dihydro-1,7-naphthyridine-7(6H)-carboxylate as a solid

Yield: 38.7%; ¹H NMR (400 MHz, DMSO-d₆): δ 8.40-8.32 (m, 2H), 7.72-7.66 (m, 2H), 7.52 (ddd, J=3.3, 7.9, 9.0 Hz, 1H), 7.17 (dd, J=4.4, 9.1 Hz, 1H), 6.91 (br d, J=4.9 Hz, 1H), 4.92-4.82 (m, 1H), 4.53 (s, 2H), 4.34 (br s, 2H), 4.26 (s, 2H), 3.82 (s, 3H), 3.58 (br t, J=5.5 Hz, 2H), 2.89-2.78 (m, 2H), 2.62 (br t, J=5.5 Hz, 2H), 2.37-2.28 (m, 2H), 1.43 (s, 9H); LCMS (Method B): Rt=1.56 min, m/z=592.2 [M+H]⁺, 94.82%.

### Step 6. 2-((4-(6-((7-(tert-Butoxycarbonyl)-5,6,7,8-tetrahydro-1,7-naphthyridin-4-yl)oxy)-2-azaspiro[3.3]heptan-2-yl)pyrimidin-5-yl)oxy)-5-fluorobenzoic Acid

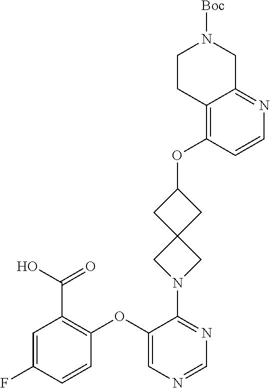 [SMILES: CC(C)(C)OC(=O)N1CCc2c(OC3CC4(C3)CN(c3ncncc3Oc3ccc(F)cc3C(=O)O)C4)ccnc2C1]

This compound was synthesized following the procedure described for the synthesis of Intermediate 24, step 4, starting with 1 equivalent of tert-butyl 4-((2-(5-(4-fluoro-2-(methoxycarbonyl)phenoxy)pyrimidin-4-yl)-2-azaspiro[3.3]heptan-6-yl)oxy)-5,8-dihydro-1,7-naphthyridine-7(6H)-carboxylate, except that 2.5 equivalents of LiOH·H₂O was used, and THF:H₂O (7:4) was used as the solvent. After completion, the reaction was concentrated under reduced pressure. The crude was dissolved in water and acidified with 1.5N HCl solution at 0° C. The precipitate was filtered, washed with water and dried under vacuum to obtain crude 2-((4-(6-((7-(tert-butoxycarbonyl)-5,6,7,8-tetrahydro-1,7-naphthyridin-4-yl)oxy)-2-azaspiro[3.3]heptan-2-yl)pyrimidin-5-yl)oxy)-5-fluorobenzoic acid as a solid. This material was used without further purification.

Yield: Quantitative; ¹H NMR (400 MHz, DMSO-d₆): δ 8.33-8.14 (m, 2H), 7.61-7.47 (m, 2H), 7.33 (br s, 1H), 7.11-6.98 (m, 1H), 6.73 (br d, J=5.4 Hz, 1H), 4.77 (quin, J=6.4 Hz, 1H), 4.44 (br s, 2H), 4.30 (br s, 2H), 4.21 (br s, 2H), 3.56 (br s, 2H), 2.85-2.75 (m, 2H), 2.63-2.56 (m, 2H), 2.31-2.22 (m, 2H), 1.42 (s, 9H), acid proton not observed; LCMS (Method A): Rt=1.42 min, m/z=578.1 [M+H]⁺, 81.33%.

### Step 7. tert-Butyl 4-((2-(5-(2-(((1r,3r)-3-((tert-butoxycarbonyl)amino)-3-methylcyclobutyl)(isopropyl)carbamyl)-4-fluorophenoxy)pyrimidin-4-yl)-2-azaspiro[3.3]heptan-6-yl)oxy)-5,8-dihydro-1,7-naphthyridine-7(6H)-carboxylate

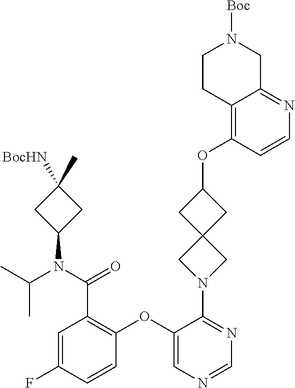 [SMILES: CC(C)N(C(=O)c1cc(F)ccc1Oc1cncnc1N1CC2(CC(Oc3ccnc4c3CCN(C(=O)OC(C)(C)C)C4)C2)C1)[C@H]1C[C@](C)(NC(=O)OC(C)(C)C)C1]

This compound was synthesized following the procedure described for the synthesis of Example 1, step 2, starting with 1 equivalent of 2-((4-(6-((7-(tert-butoxycarbonyl)-5,6,7,8-tetrahydro-1,7-naphthyridin-4-yl)oxy)-2-azaspiro[3.3]heptan-2-yl)pyrimidin-5-yl)oxy)-5-fluorobenzoic acid and 1 equivalent of tert-butyl ((1r,3r)-3-(isopropylamino)-1-methylcyclobutyl)carbamate. After work-up, the crude was purified by reverse phase chromatography using a SiliCycle SiliaSep C18 column (40 g) on Biotage isolera one (Mobile phase A: 10 mM NH₄HCO₃ in water, B: ACN) to obtain tert-butyl 4-((2-(5-(2-(((1r,3r)-3-((tert-butoxycarbonyl)amino)-3-methylcyclobutyl)(isopropyl)carbamoyl)-4-fluorophenoxy)pyrimidin-4-yl)-2-azaspiro[3.3]heptan-6-yl)oxy)-5,8-dihydro-1,7-naphthyridine-7(6H)-carboxylate as a solid.

Yield: 32.2%; LCMS (Method A): Rt=2.30 min, m/z=802.3 [M+H]⁺, 99.28%.

### Step 8. N-((1r,3r)-3-Amino-3-methylcyclobutyl)-5-fluoro-N-isopropyl-2-((4-(6-((5,6,7,8-tetrahydro-1,7-naphthyridin-4-yl)oxy)-2-azaspiro[3.3]heptan-2-y)pyrimidin-5-yl)oxy)benzamide xHCl

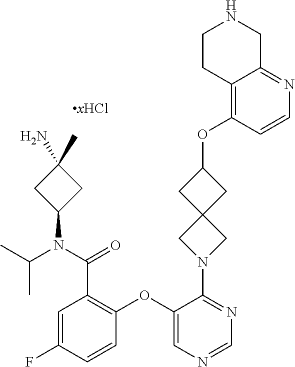 [SMILES: CC(C)N(C(=O)c1cc(F)ccc1Oc1cncnc1N1CC2(CC(Oc3ccnc4c3CCNC4)C2)C1)[C@H]1C[C@](C)(N)C1]

This compound was synthesized following the procedure described for the synthesis of Example 20, step 3, starting with 1 equivalent of tert-butyl 4-((2-(5-(2-(((1r,3r)-3-((tert-butoxycarbonyl)amino)-3-methylcyclobutyl)(isopropyl)carbamoyl)-4-fluorophenoxy)pyrimidin-4-yl)-2-azaspiro[3.3]heptan-6-yl)oxy)-5,8-dihydro-1,7-naphthyridine-7(6H)-carboxylate, except that 8 equivalents of TMSCl was used.

After completion, the reaction was concentrated under reduced pressure to obtain crude N-((1r,3r)-3-amino-3-methylcyclobutyl)-5-fluoro-N-isopropyl-2-((4-(6-((5,6,7,8-tetrahydro-1,7-naphthyridin-4-yl)oxy)-2-azaspiro[3.3]heptan-2-yl)pyrimidin-5-yl)oxy)benzamide xHCl as a gummy solid. This material was used without further purification.

Yield: 97%; LCMS (Method H): Rt=1.01 min, m/z=602.3 [M+H]⁺, 81.44%.

### Step 9. N-((1r,3r)-3-(Dimethylamino)-3-methylcyclobutyl)-5-fluoro-N-isopropyl-2-((4-(6-((7-methyl-5,6,7,8-tetrahydro-1,7-naphthyridin-4-yl)oxy)-2-azaspiro[3.3]heptan-2-yl)pyrimidin-5-yl)oxy)benzamide

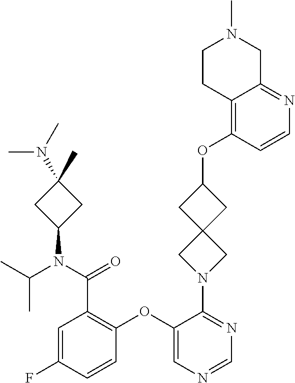 [SMILES: CC(C)N(C(=O)c1cc(F)ccc1Oc1cncnc1N1CC2(CC(Oc3ccnc4c3CCN(C)C4)C2)C1)[C@H]1C[C@](C)(N(C)C)C1]

This compound was synthesized following the procedure described for the synthesis of Example 76, starting with 1 equivalent of N-((1r,3r)-3-amino-3-methylcyclobutyl)-5-fluoro-N-isopropyl-2-((4-(6-((5,6,7,8-tetrahydro-1,7-naphthyridin-4-yl)oxy)-2-azaspiro[3.3]heptan-2-yl)pyrimidin-5-yl)oxy)benzamide xHCl, except that 20 equivalents of formaldehyde (37% in H₂O), 0.2 equivalent of AcOH, and 4 equivalents of STAB were used. After completion, the reaction mixture was diluted with water and the mixture was extracted with 10% MeOH in DCM. The combined organic layer was washed with brine, dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain N-((1r,3r)-3-(dimethylamino)-3-methylcyclobutyl)-5-fluoro-N-isopropyl-2-((4-(6-((7-methyl-5,6,7,8-tetrahydro-1,7-naphthyridin-4-yl)oxy)-2-azaspiro[3.3]heptan-2-yl)pyrimidin-5-yl)oxy)benzamide as a solid.

Yield: 55.9%; ¹H NMR (400 MHz, DMSO-d₆): δ 8.31-8.23 (m, 1H), 8.16 (d, J=5.4 Hz, 1H), 7.79-7.59 (m, 1H), 7.31-7.03 (m, 3H), 6.70-6.61 (m, 1H), 4.75 (quin, J=6.5 Hz, 1H), 4.34-4.07 (m, 4H), 4.06-3.75 (m, 1H), 3.74-3.65 (m, 1H), 3.43 (s, 2H), 2.83-2.75 (m, 2H), 2.65-2.54 (m, 4H), 2.34 (s, 3H), 2.30-2.20 (m, 2H), 2.15-1.92 (m, 8H), 1.80-1.69 (m, 1H), 1.47-1.27 (m, 3H), 1.15-0.90 (m, 6H), one proton merged with solvent peaks; LCMS (Method A): Rt=1.71 min, m/z=644.3 [M+H]⁺; HPLC (Method C): Rt=8.69 min, 99.74%; Chiral SFC (Method U): Rt=1.24 min, 100%.

### Example 202. (D1)-5-Fluoro-2-((5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-isopropyl-N-((2s,4r)-5-methyl-5-azaspiro[3.4]octan-2-yl)benzamide (Compound No. 202)

### Example 203. (D2)-5-Fluoro-2-((5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-isopropyl-N-((2s,4r)-5-methyl-5-azaspiro[3.4]octan-2-yl)benzamide (Compound No. 203)

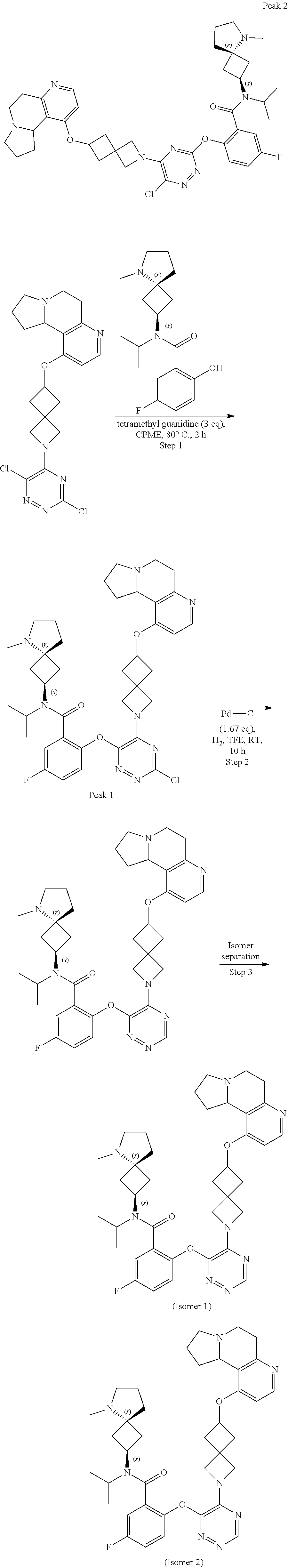

### Step 1. 2-((3-Chloro-5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-5-fluoro-N-isopropyl-N-((2s,4r)-5-methyl-5-azaspiro[3.4]octan-2-yl)benzamide (diastereomeric mixture) and 2-((6-chloro-5-(6-((5,6,8,9,10,10α-hexahydropyrrolo[2,1-f][1,6]naphthyridin-1-yl)oxy)-2-azaspiro[3.3]heptan-2-yl)-1,2,4-triazin-3-yl)oxy)-5-fluoro-N-isopropyl-N-((2s,4r)-5-methyl-5-azaspiro[3.4]octan-2-yl)benzamide (Diastereomeric Mixture)

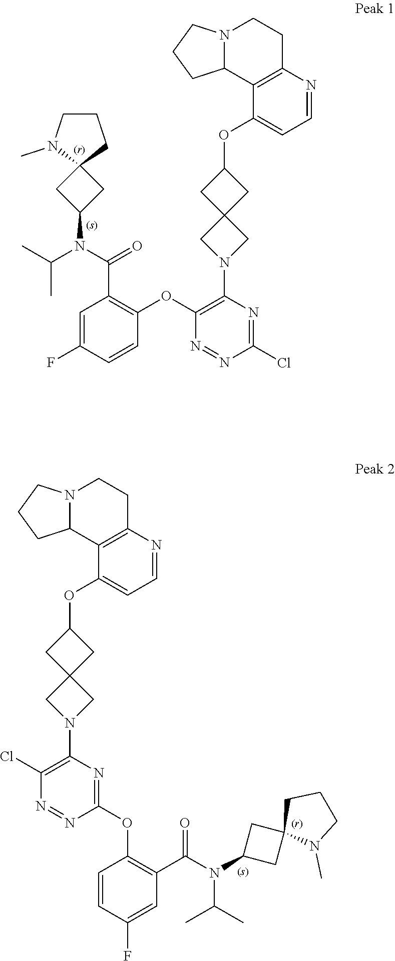 [SMILES: CC(C)N(C(=O)c1cc(F)ccc1Oc1nnc(Cl)nc1N1CC2(CC(Oc3ccnc4c3C3CCCN3CC4)C2)C1)[C@H]1C[C@@]2(CCCN2C)C1 ; CC(C)N(C(=O)c1cc(F)ccc1Oc1nnc(Cl)c(N2CC3(CC(Oc4ccnc5c4C4CCCN4CC5)C3)C2)n1)[C@H]1C[C@@]2(CCCN2C)C1]

This compound was synthesized following the procedure described for the synthesis of Example 164, step 2, starting with 1 equivalent of (±)-1-((2-(3,6-dichloro-1,2,4-triazin-5-yl)-2-azaspiro[3.3]heptan-6-yl)oxy)-5,6,8,9,10,10a-hexahydropyrrolo[2,1-f][1,6]naphthyridine and 1 equivalent of 5-fluoro-2-hydroxy-N-isopropyl-N-((2s,4r)-5-methyl-5-azaspiro[3.4]octan-2-yl)benzamide. The reaction was diluted with water and the mixture was extracted with EtOAc. The combined organic layer was washed with brine, dried over anhydrous sodium sulfate and filtered, and the filtrate was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN), then by achiral Prep SFC (Method B) to obtain both regioisomers.

Peak 1. 2-((3-Chloro-5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-5-fluoro-N-isopropyl-N-((2s,4r)-5-methyl-5-azaspiro[3.4]octan-2-yl)benzamide (diastereomeric mixture) as a solid.

Yield: 17.75%; ¹H NMR (400 MHz, DMSO-d₆): δ 8.23-8.12 (m, 1H), 7.51-7.12 (m, 3H), 6.67 (br s, 1H), 4.86-4.56 (m, 3H), 4.32-4.14 (m, 2H), 3.94-3.50 (m, 3H), 3.02-2.60 (m, 9H), 2.47-2.15 (m, 7H), 2.01-1.65 (m, 7H), 1.60-1.22 (m, 6H), 1.15-0.63 (m, 3H), one proton merged with solvent peaks; LCMS (Method H): Rt=1.16 min, m/z=717.3 [M+H]⁺; HPLC (Method G): Rt=7.12 min, 96.52%.

Peak 2. 2-((6-Chloro-5-(6-((5,6,8,9,10,10a-hexahydropyrrolo[2,1-f][1,6]naphthyridin-1-yl)oxy)-2-azaspiro[3.3]heptan-2-yl)-1,2,4-triazin-3-yl)oxy)-5-fluoro-N-isopropyl-N-((2s,4r)-5-methyl-5-azaspiro[3.4]octan-2-yl)benzamide (diastereomeric mixture) as a solid.

Yield: 4.68%; ¹H NMR (400 MHz, DMSO-d₆): δ 8.22-8.13 (m, 1H), 7.50-7.10 (m, 3H), 6.68 (br s, 1H), 4.84-4.62 (m, 3H), 4.30-3.97 (m, 2H), 3.77-3.59 (m, 3H), 3.04-2.64 (m, 9H), 2.45-2.16 (m, 7H), 2.05-1.64 (m, 7H), 1.63-1.40 (m, 4H), 1.30-0.87 (m, 5H), one proton merged with solvent peaks; LCMS (Method H): Rt=1.19 min, m/z=717.3 [M+H]⁺; HPLC (Method G): Rt=7.18 min, 89.12%.

### Step 2. 5-Fluoro-2-((5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-N-isopropyl-N-((2s,4r)-5-methyl-5-azaspiro[3.4]octan-2-yl)benzamide (Diastereomeric Mixture)

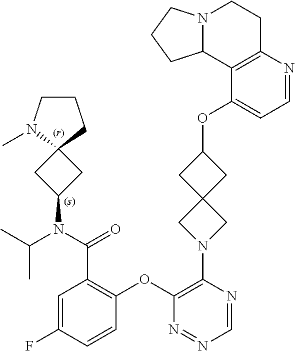 [SMILES: CC(C)N(C(=O)c1cc(F)ccc1Oc1nncnc1N1CC2(CC(Oc3ccnc4c3C3CCCN3CC4)C2)C1)[C@H]1C[C@@]2(CCCN2C)C1]

In a 50 mL round bottom flask, 2-((3-chloro-5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-5-fluoro-N-isopropyl-N-((2s,4r)-5-methyl-5-azaspiro[3.4]octan-2-yl)benzamide (diastereomeric mixture) (350 mg, 0.488 mmol) was dissolved in TFE (5 mL). Nitrogen gas was bubbled through the solution for 5 min. To this reaction mixture, Pd—C (350 mg, 0.329 mmol) was added under a nitrogen atmosphere. The reaction mixture was stirred under a hydrogen atmosphere (balloon pressure) at RT for 5 h. The reaction was monitored by LCMS (46% pdt and 36% SM was observed). The reaction was filtered through a Celite® pad, and the pad was washed with MeOH (2×50 mL). The filtrate was concentrated under reduced pressure. This crude was dissolved in TFE (5 mL) and Pd—C (176 mg, 0.166 mmol) was added under a nitrogen atmosphere. The reaction mixture was stirred under a hydrogen atmosphere (balloon pressure) at RT for 5 h. The reaction was monitored by TLC (10% MeOH in DCM) and LCMS. After completion, the reaction was filtered through a Celite® pad, and the pad was washed with MeOH (50 mL). The filtrate was concentrated under reduced pressure to obtain the crude diastereomeric mixture of 5-fluoro-2-((5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-N-isopropyl-N-((2s,4r)-5-methyl-5-azaspiro[3.4]octan-2-yl)benzamide (280 mg) as a sticky solid. This material was used without further purification. LCMS (Method H): Rt=1.03 min, m/z=683.3 [M+H]⁺; HPLC (Method G): Rt=6.41 min, 92.72%; Chiral SFC (Method P): Peak 1: Rt=6.28 min, 48.37%; Peak 2: Rt=8.16 min, 49.18%.

### Step 3. (D1)-5-Fluoro-2-((5-(6-((5,6,8,9,10,10α-hexahydropyrrolo[2,1-f][1,6]naphthyridin-5-yl)oxy)-2-azaspiro[3.3]heptan-2-yl)-1,2,4-triazin-6-yl)oxy)-N-isopropyl-N-((2s,4r)-5-methyl-5-azaspiro[3.4]octan-2-yl)benzamide and (D2)-5-fluoro-2-((5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-isopropyl-N-((2s,4r)-5-methyl-5-azaspiro[3.4]octan-2-yl)benzamide

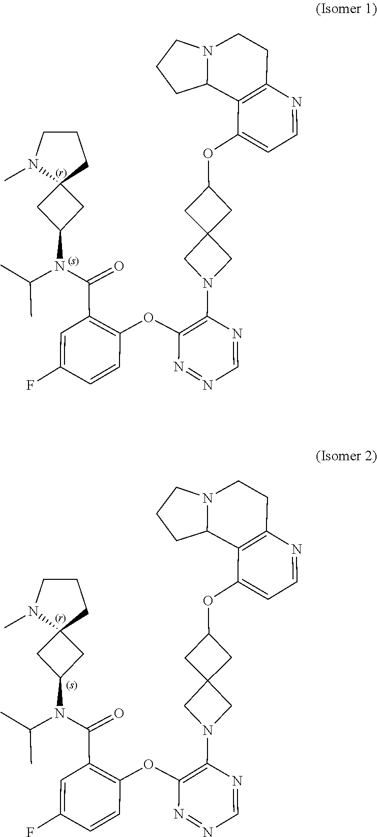 [SMILES: CC(C)N(C(=O)c1cc(F)ccc1Oc1nncnc1N1CC2(CC(Oc3ccnc4c3C3CCCN3CC4)C2)C1)[C@H]1C[C@@]2(CCCN2C)C1 ; CC(C)N(C(=O)c1cc(F)ccc1Oc1nncnc1N1CC2(CC(Oc3ccnc4c3C3CCCN3CC4)C2)C1)[C@H]1C[C@@]2(CCCN2C)C1]

The diastereomeric mixture of 5-fluoro-2-((5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-N-isopropyl-N-((2s,4r)-5-methyl-5-azaspiro[3.4]octan-2-yl)benzamide (280 mg, 0.410 mmol) was purified by chiral Prep SFC (Method P). The pure fractions were concentrated under reduced pressure and lyophilized to obtain (D1)-5-fluoro-2-((5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-isopropyl-N-((2s,4r)-5-methyl-5-azaspiro[3.4]octan-2-yl)benzamide (49 mg, 16.94% yield) and (D2)-5-fluoro-2-((5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-N-isopropyl-N-((2s,4r)-5-methyl-5-azaspiro[3.4]octan-2-yl)benzamide (38 mg, 12.96% yield) as solids.

Isomer 1. ¹H NMR (400 MHz, DMSO-d₆): δ 8.46 (s, 1H), 8.22-8.14 (m, 1H), 7.50-7.11 (m, 3H), 6.74-6.58 (m, 1H), 4.88-4.53 (m, 3H), 4.29-3.82 (m, 3H), 3.71-3.49 (m, 3H), 3.03-2.59 (m, 9H), 2.45-2.15 (m, 7H), 2.01-1.62 (m, 7H), 1.59-1.18 (m, 6H), 1.12-0.52 (m, 3H); LCMS (Method H): Rt=1.05 min, m/z=683.3 [M+H]⁺; HPLC (Method G): Rt=6.53 min, 96.81%; Chiral SFC (Method P): Rt=5.20 min, 98.41%.

Isomer 2. ¹H NMR (400 MHz, DMSO-d₆): δ 8.46 (s, 1H), 8.18 (br d, J=5.1 Hz, 1H), 7.50-7.13 (m, 3H), 6.73-6.60 (m, 1H), 4.85-4.51 (m, 3H), 4.31-3.80 (m, 3H), 3.72-3.48 (m, 3H), 3.03-2.61 (m, 9H), 2.44-2.16 (m, 7H), 2.00-1.64 (m, 7H), 1.61-1.19 (m, 6H), 1.11-0.54 (m, 3H); LCMS (Method H): Rt=1.05 min, m/z=683.4 [M+H]⁺; HPLC (Method C): Rt=8.05 min, 95.47%; Chiral SFC (Method P): Rt=7.92 min, 98.86%.

### Example 204. (D1)-N-((1r,3r)-3-(Azetidin-1-yl)-3-methylcyclobutyl)-5-fluoro-2-((5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-isopropylbenzamide (Compound No. 204)

### Example 205. (D2)-N-((1r,3r)-3-(Azetidin-1-yl)-3-methylcyclobutyl)-5-fluoro-2-((5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-isopropylbenzamide (Compound No. 205)

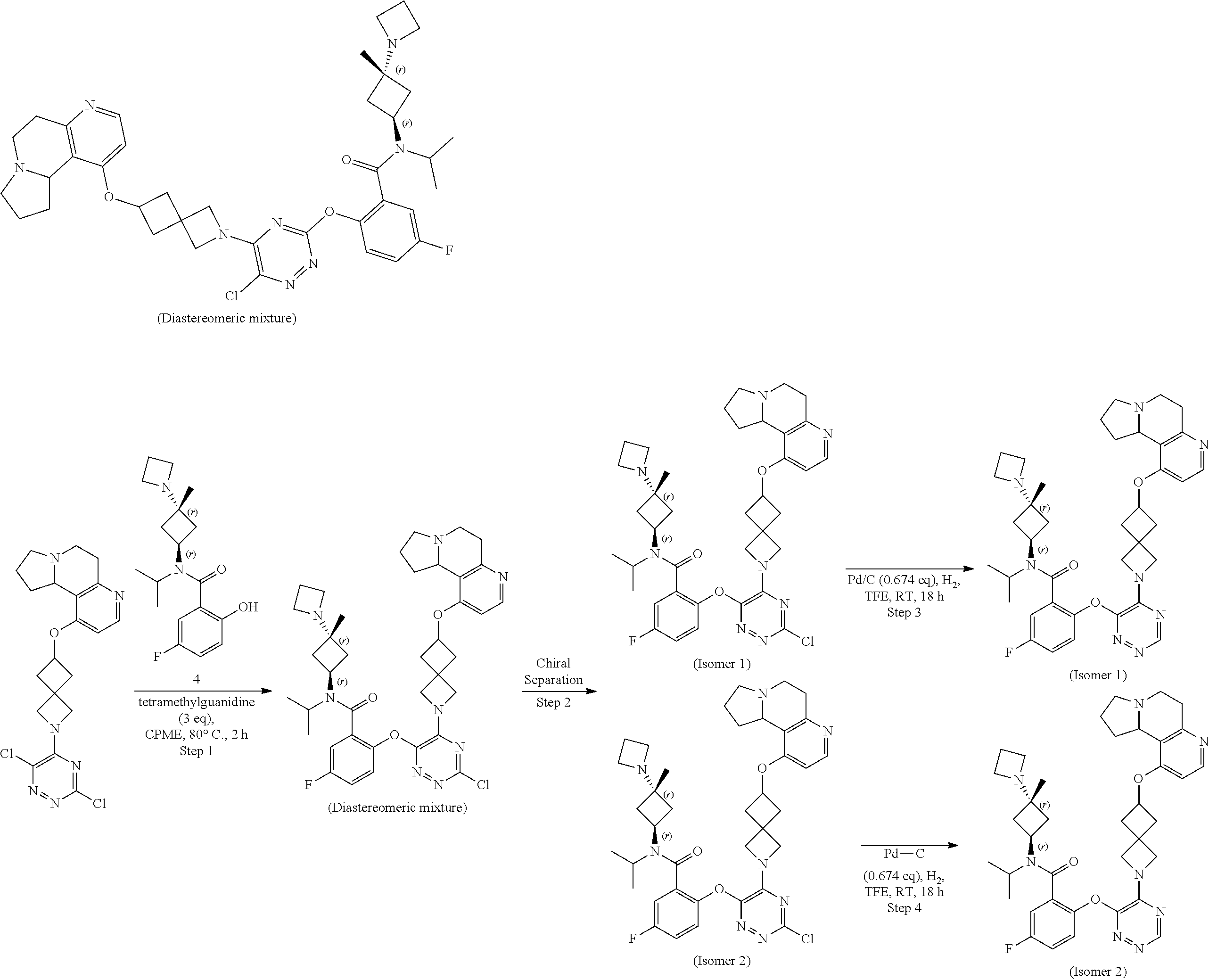

### Step 1. N-((1r,3r)-3-(Azetidin-1-yl)-3-methylcyclobutyl)-2-((3-chloro-5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-5-fluoro-N-isopropylbenzamide (diastereomeric mixture) and N-((1r,3r)-3-(azetidin-1-yl)-3-methylcyclobutyl)-2-((6-chloro-5-(6-((5,6,8,9,10,10α-hexahydropyrrolo[2,1-f][1,6]naphthyridin-1-yl)oxy)-2-azaspiro[3.3]heptan-2-yl)-1,2,4-triazin-3-yl)oxy)-5-fluoro-N-isopropylbenzamide (Diastereomeric Mixture)

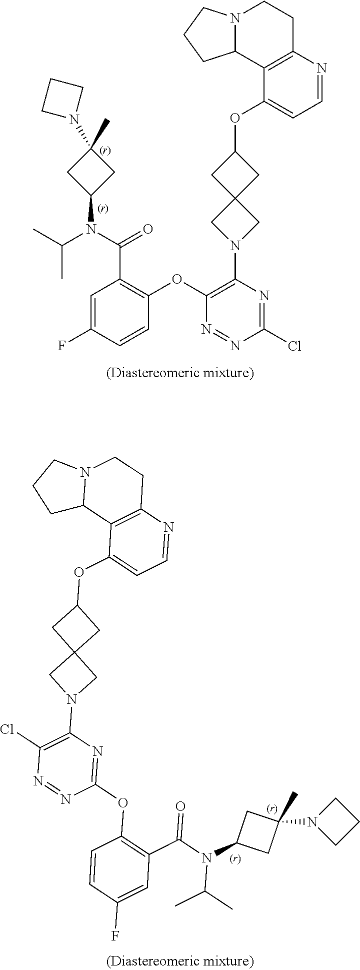 [SMILES: CC(C)N(C(=O)c1cc(F)ccc1Oc1nnc(Cl)nc1N1CC2(CC(Oc3ccnc4c3C3CCCN3CC4)C2)C1)[C@H]1C[C@](C)(N2CCC2)C1 ; CC(C)N(C(=O)c1cc(F)ccc1Oc1nnc(Cl)c(N2CC3(CC(Oc4ccnc5c4C4CCCN4CC5)C3)C2)n1)[C@H]1C[C@](C)(N2CCC2)C1]

This compound was synthesized following the procedure described for the synthesis of Example 164, step 2, starting with 1 equivalent of 1-((2-(3,6-dichloro-1,2,4-triazin-5-yl)-2-azaspiro[3.3]heptan-6-yl)oxy)-5,6,8,9,10,10a-hexahydropyrrolo[2,1-f][1,6]naphthyridine and 1 equivalent of N-((1r,3r)-3-(azetidin-1-yl)-3-methylcyclobutyl)-5-fluoro-2-hydroxy-N-isopropylbenzamide. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain a mixture of diastereomers of N-((1r,3r)-3-(azetidin-1-yl)-3-methylcyclobutyl)-2-((3-chloro-5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-5-fluoro-N-isopropylbenzamide and N-((1r,3r)-3-(azetidin-1-yl)-3-methylcyclobutyl)-2-((6-chloro-5-(6-((5,6,8,9,10,10α-hexahydropyrrolo[2,1-f][1,6]naphthyridin-1-yl)oxy)-2-azaspiro[3.3]heptan-2-yl)-1,2,4-triazin-3-yl)oxy)-5-fluoro-N-isopropylbenzamide as a solid.

Yield: 16.33%; LCMS (Method B): Rt=1.03-1.13 min, m/z=717.4 [M+H]⁺, 98.33%. HPLC (Method G): Rt=6.75 min, 99.38%. Chiral SFC (Method AAN): Peak 1: Rt=4.52 min, 44.54%; Peak 2: Rt=6.64 min, 43.44%; Peak 3: Rt=8.73 min, 5.71%; Peak 4: Rt=13.30 min, 5.57%.

### Step 2. (D1)-N-((1r,3r)-3-(Azetidin-1-yl)-3-methylcyclobutyl)-2-((3-chloro-5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-5-fluoro-N-isopropylbenzamide & (D2)-N-((1r,3r)-3-(Azetidin-1-yl)-3-methylcyclobutyl)-2-((3-chloro-5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-5-fluoro-N-isopropylbenzamide and (D3)-N-((1r,3r)-3-(azetidin-1-yl)-3-methylcyclobutyl)-2-((6-chloro-5-(6-((5,6,8,9,10,10α-hexahydropyrrolo[2,1-f][1,6]naphthyridin-1-yl)oxy)-2-azaspiro[3.3]heptan-2-yl)-1,2,4-triazin-3-yl)oxy)-5-fluoro-N-isopropylbenzamide & (D4)-N-((1r,3r)-3-(azetidin-1-yl)-3-methylcyclobutyl)-2-((6-chloro-5-(6-((5,6,8,9,10,10α-hexahydropyrrolo[2,1-f][1,6]naphthyridin-1-yl)oxy)-2-azaspiro[3.3]heptan-2-yl)-1,2,4-triazin-3-yl)oxy)-5-fluoro-N-isopropylbenzamide

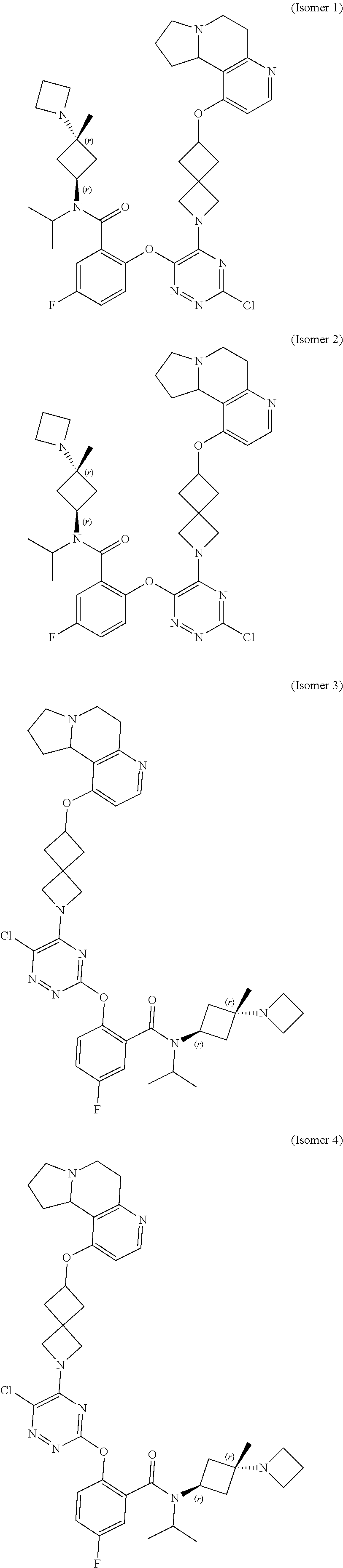 [SMILES: CC(C)N(C(=O)c1cc(F)ccc1Oc1nnc(Cl)nc1N1CC2(CC(Oc3ccnc4c3C3CCCN3CC4)C2)C1)[C@H]1C[C@](C)(N2CCC2)C1 ; CC(C)N(C(=O)c1cc(F)ccc1Oc1nnc(Cl)nc1N1CC2(CC(Oc3ccnc4c3C3CCCN3CC4)C2)C1)[C@H]1C[C@](C)(N2CCC2)C1 ; CC(C)N(C(=O)c1cc(F)ccc1Oc1nnc(Cl)c(N2CC3(CC(Oc4ccnc5c4C4CCCN4CC5)C3)C2)n1)[C@H]1C[C@](C)(N2CCC2)C1 ; CC(C)N(C(=O)c1cc(F)ccc1Oc1nnc(Cl)c(N2CC3(CC(Oc4ccnc5c4C4CCCN4CC5)C3)C2)n1)[C@H]1C[C@](C)(N2CCC2)C1]

A mixture of diastereomers of N-((1r,3r)-3-(azetidin-1-yl)-3-methylcyclobutyl)-2-((3-chloro-5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-5-fluoro-N-isopropylbenzamide and N-((1r,3r)-3-(azetidin-1-yl)-3-methylcyclobutyl)-2-((6-chloro-5-(6-((5,6,8,9,10,10a-hexahydropyrrolo[2,1-f][1,6]naphthyridin-1-yl)oxy)-2-azaspiro[3.3]heptan-2-yl)-1,2,4-triazin-3-yl)oxy)-5-fluoro-N-isopropylbenzamide (880 mg, 1.227 mmol) was purified by chiral Prep SFC (Method S) to obtain the major diastereomers (D1)-N-((1r,3r)-3-(azetidin-1-yl)-3-methylcyclobutyl)-2-((3-chloro-5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-5-fluoro-N-isopropylbenzamide (310 mg, 10.00% yield) and (D2)-N-((1r,3r)-3-(azetidin-1-yl)-3-methylcyclobutyl)-2-((3-chloro-5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-5-fluoro-N-isopropylbenzamide (300 mg, 9.29% yield).

Isomer 1. ¹H NMR (400 MHz, DMSO-d₆): δ 8.18 (t, J=4.8 Hz, 1H), 7.52-7.14 (m, 3H), 6.71-6.61 (m, 1H), 4.78 (quin, J=6.3 Hz, 1H), 4.74-4.58 (m, 2H), 4.32-4.17 (m, 2H), 3.95-3.50 (m, 3H), 3.14-3.05 (m, 2H), 2.99-2.73 (m, 8H), 2.73-2.60 (m, 2H), 2.44-2.22 (m, 2H), 2.17-1.98 (m, 2H), 1.95-1.60 (m, 5H), 1.56-1.37 (m, 3H), 1.30-0.63 (m, 9H); Chiral SFC (Method AAN): Rt=4.38 min, 97.86%.

Isomer 2. ¹H NMR (400 MHz, DMSO-d₆): δ 8.18 (t, J=4.8 Hz, 1H), 7.50-7.14 (m, 3H), 6.73-6.60 (m, 1H), 4.78 (quin, J=6.2 Hz, 1H), 4.73-4.59 (m, 2H), 4.30-4.18 (m, 2H), 3.94-3.47 (m, 3H), 3.14-3.05 (m, 2H), 3.01-2.78 (m, 8H), 2.76-2.60 (m, 2H), 2.45-2.21 (m, 2H), 2.18-2.00 (m, 2H), 1.92-1.62 (m, 5H), 1.57-1.37 (m, 3H), 1.31-0.62 (m, 9H); LCMS (Method A): Rt=2.11 min, m/z=717.2 [M+H]⁺, 82.10%. Chiral SFC (Method AAN): Rt=6.40 min, 100%.

Note: Minor diastereomers were not isolated.

### Step 3. (D1)-N-((1r,3r)-3-(Azetidin-1-yl)-3-methylcyclobutyl)-5-fluoro-2-((5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-N-isopropylbenzamide

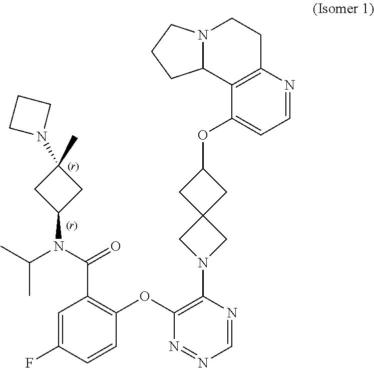 [SMILES: CC(C)N(C(=O)c1cc(F)ccc1Oc1nncnc1N1CC2(CC(Oc3ccnc4c3C3CCCN3CC4)C2)C1)[C@H]1C[C@](C)(N2CCC2)C1]

This compound was synthesized following the procedure described for the synthesis of Example 59, step 5, starting with 1 equivalent (D1)-N-((1r,3r)-3-(azetidin-1-yl)-3-methylcyclobutyl)-2-((3-chloro-5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-5-fluoro-N-isopropylbenzamide, except that 0.674 equivalent of Pd—C was used. After completion, the reaction was filtered through a Celite® pad, and the pad was washed with MeOH. The filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to obtain (D1)-N-((1r,3r)-3-(azetidin-1-yl)-3-methylcyclobutyl)-5-fluoro-2-((5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-isopropylbenzamide as a solid.

Yield: 53.9%; ¹H NMR (400 MHz, DMSO-d₆): δ 8.46 (br s, 1H), 8.18 (d, J=5.5 Hz, 1H), 7.49-7.13 (m, 3H), 6.71-6.62 (m, 1H), 4.80 (quin, J=6.4 Hz, 1H), 4.72-4.51 (m, 2H), 4.29-4.09 (m, 2H), 3.97-3.50 (m, 3H), 3.15-3.02 (m, 2H), 3.01-2.60 (m, 10H), 2.47-2.21 (m, 3H), 2.16-2.00 (m, 2H), 1.93-1.68 (m, 5H), 1.66-1.36 (m, 3H), 1.28-0.54 (m, 8H); LCMS (Method H): Rt=1.01 min, m/z=683.3 [M+H]⁺; HPLC (Method G): Rt=6.41 min, 99.46%; Chiral SFC (Method J): Rt=7.27 min, 99.19%.

### Step 4. (D2)-N-((1r,3r)-3-(Azetidin-1-yl)-3-methylcyclobutyl)-5-fluoro-2-((5-(6-((5,6,8,9,10,10α-hexahydropyrrolo[2,1-f][1,6]naphthyridin-1-yl)oxy)-2-azaspiro[3.3]heptan-2-yl)-1,2,4-triazin-6-yl)oxy)-N-isopropylbenzamide

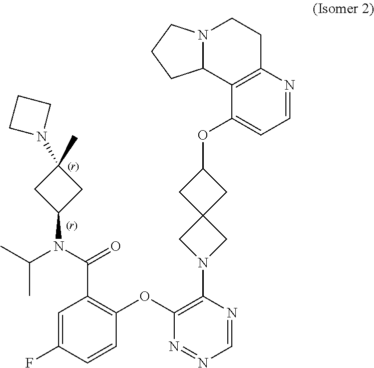 [SMILES: CC(C)N(C(=O)c1cc(F)ccc1Oc1nncnc1N1CC2(CC(Oc3ccnc4c3C3CCCN3CC4)C2)C1)[C@H]1C[C@](C)(N2CCC2)C1]

This compound was synthesized following the procedure described for the synthesis of Example 59, step 5, starting with 1 equivalent (D2)-N-((1r,3r)-3-(azetidin-1-yl)-3-methylcyclobutyl)-2-((3-chloro-5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-5-fluoro-N-isopropylbenzamide, except that 0.674 equivalent of Pd—C was used. After completion, the reaction was filtered through a Celite® pad, and the pad was washed with MeOH (45 mL). The filtrate was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure and lyophilized to obtain (D2)-N-((1r,3r)-3-(azetidin-1-yl)-3-methylcyclobutyl)-5-fluoro-2-((5-(6-((5,6,8,9,10,10a-hexahydropyrrolo[2,1-f][1,6]naphthyridin-1-yl)oxy)-2-azaspiro[3.3]heptan-2-yl)-1,2,4-triazin-6-yl)oxy)-N-isopropylbenzamide as a solid.

Yield: 58.6%; ¹H NMR (400 MHz, DMSO-d₆): δ 8.46 (br s, 11H), 8.18 (d, J=5.5 Hz, 1H), 7.50-7.11 (m, 3H), 6.75-6.59 (m, 1H), 4.86-4.75 (m, 1H), 4.73-4.50 (m, 2H), 4.31-4.07 (m, 2H), 3.97-3.51 (m, 3H), 3.17-3.02 (m, 2H), 3.01-2.59 (m, 10H), 2.45-2.23 (m, 3H), 2.17-2.99 (m, 2H), 1.92-1.69 (m, 5H), 1.61-1.38 (m, 3H), 1.29-0.55 (m, 8H); LCMS (Method H): Rt=1.04 min, m/z=683.4 [M+H]⁺, HPLC (Method G): Rt=6.41 min, 98.36%; Chiral SFC (Method A): Rt=3.30 min, 98.03%.

### Example 206. (D1)-5-Fluoro-N-isopropyl-2-((5-(6-((6-methyl-5,6,7,8-tetrahydro-1,6-naphthyridin-4-yl)oxy)-2-azaspiro[3.3]heptan-2-yl)-1,2,4-triazin-6-yl)oxy)-N-(5-methyl-5-azaspiro[3.5]nonan-2-yl)benzamide (Compound No. 206)

### Example 207. (D2)-5-Fluoro-N-isopropyl-2-((5-(6-((6-methyl-5,6,7,8-tetrahydro-1,6-naphthyridin-4-yl)oxy)-2-azaspiro[3.3]heptan-2-yl)-1,2,4-triazin-6-yl)oxy)-N-(5-methyl-5-azaspiro[3.5]nonan-2-yl)benzamide (Compound No. 207)

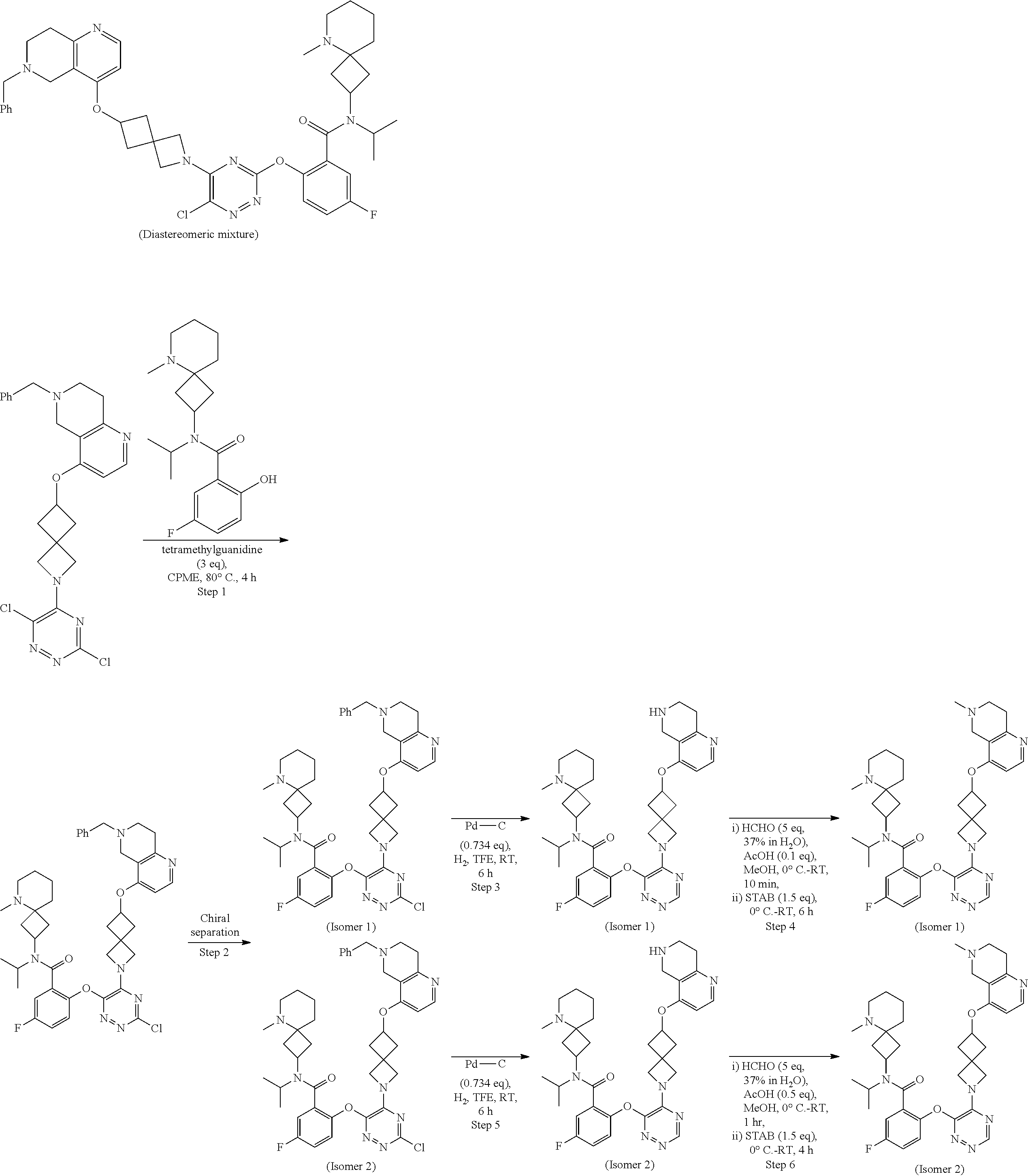

### Step 1. 2-(5-(6-(6-Benzyl-5,6,7,8-tetrahydro-, 6-naphthyridin-4-yl)oxy)-2-azaspiro[3.3]heptan-2-yl)-3-chloro-1,2,4-triazin-6-yl)oxy)-5-fluoro-N-isopropyl-N-(5-methyl-5-azaspiro[3.5]nonan-2-yl)benzamide (diastereomeric mixture)) and 2-((5-(6-((6-benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-(5-methyl-5-azaspiro[3.5]nonan-2-yl)benzamide (Diastereomeric Mixture)

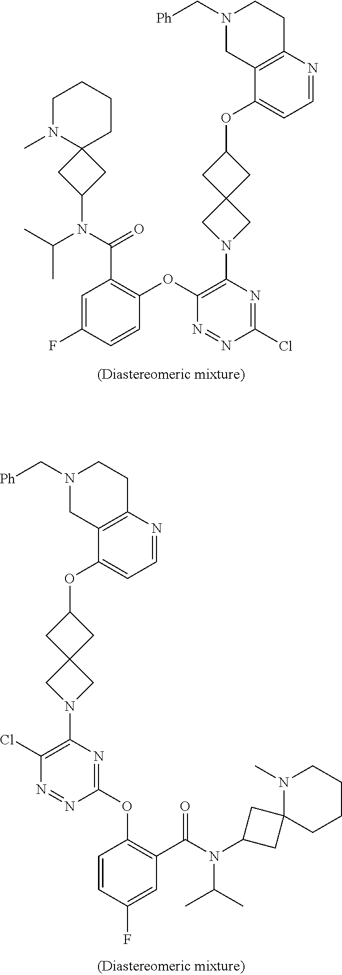 [SMILES: CC(C)N(C(=O)c1cc(F)ccc1Oc1nnc(Cl)nc1N1CC2(CC(Oc3ccnc4c3CN(Cc3ccccc3)CC4)C2)C1)C1CC2(CCCCN2C)C1 ; CC(C)N(C(=O)c1cc(F)ccc1Oc1nnc(Cl)c(N2CC3(CC(Oc4ccnc5c4CN(Cc4ccccc4)CC5)C3)C2)n1)C1CC2(CCCCN2C)C1]

This compound was synthesized following the procedure described for the synthesis of Example 164, step 2, starting with 1 equivalent of 6-benzyl-4-((2-(3,6-dichloro-1,2,4-triazin-5-yl)-2-azaspiro[3.3]heptan-6-yl)oxy)-5,6,7,8-tetrahydro-1,6-naphthyridine and 1 equivalent of 5-fluoro-2-hydroxy-N-isopropyl-N-(5-methyl-5-azaspiro[3.5]nonan-2-yl)benzamide. After completion, the reaction was diluted with 10% NaHCO₃ solution and the mixture was extracted with 10% MeOH in DCM. The combined organic layer was dried over anhydrous sodium sulfate and filtered, and the filtrate was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN) to obtain a mixture of diastereomers of 2-((5-(6-((6-benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-(5-methyl-5-azaspiro[3.5]nonan-2-yl)benzamide and 2-((5-(6-((6-benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-(5-methyl-5-azaspiro[3.5]nonan-2-yl)benzamide as a solid.

Yield: 39.1%; LCMS (Method H): Rt=1.31 min, m/z=781.3 [M+H]⁺; HPLC (Method C): Rt=11.57 min, 99.31%; Chiral SFC (Method AAT): Peak 1: Rt=4.22 min, 59.20%; Peak 2:Rt=6.37 min, 28.49%; Peak 3: Rt=11.15 min, 9%; Peak 4: Rt=12.75 min, 2.68%.

### Step 2. (D1)-2-((5-(6-((6-Benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-(5-methyl-5-azaspiro[3.5]nonan-2-yl)benzamide, (12)-2-((5-(6-((6-benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-(5-methyl-5-azaspiro[3.5]nonan-2-yl)benzamide, (D3)-2-((5-(6-((6-benzyl-5,6,7,8-tetrahydro-1,6-naphthyridin-4-yl)oxy)-2-azaspiro[3.3]heptan-2-yl)-6-chloro-1,2,4-triazin-3-yl)oxy)-5-fluoro-N-isopropyl-N-(5-methyl-5-azaspiro[3.5]nonan-2-yl)benzamide and (D4)-2-((5-(6-((6-benzyl-5,6,7,8-tetrahydro-1,6-naphthyridin-4-yl)oxy)-2-azaspiro[3.3]heptan-2-yl)-6-chloro-1,2,4-triazin-3-yl)oxy)-5-fluoro-N-isopropyl-N-(5-methyl-5-azaspiro[3.5]nonan-2-yl)benzamide

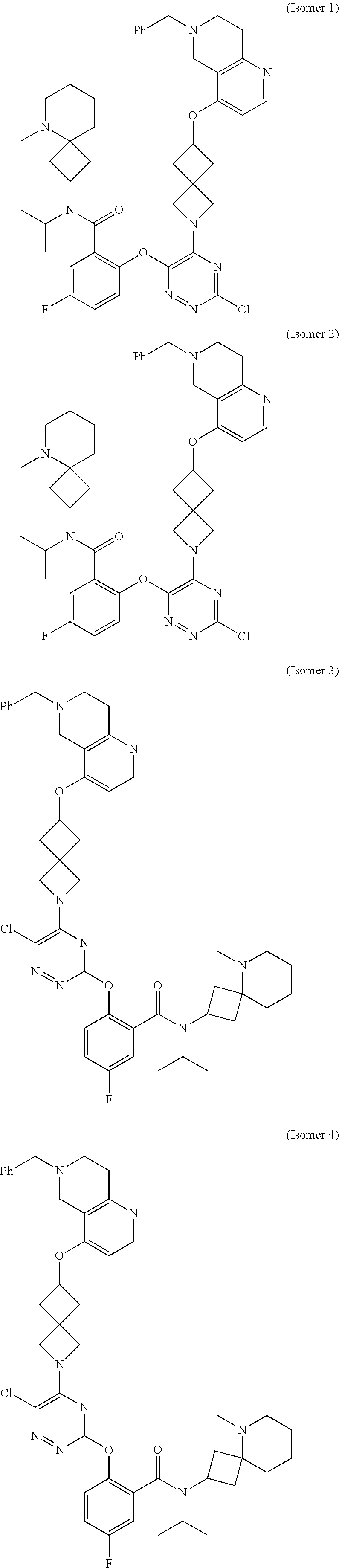 [SMILES: CC(C)N(C(=O)c1cc(F)ccc1Oc1nnc(Cl)nc1N1CC2(CC(Oc3ccnc4c3CN(Cc3ccccc3)CC4)C2)C1)C1CC2(CCCCN2C)C1 ; CC(C)N(C(=O)c1cc(F)ccc1Oc1nnc(Cl)nc1N1CC2(CC(Oc3ccnc4c3CN(Cc3ccccc3)CC4)C2)C1)C1CC2(CCCCN2C)C1 ; CC(C)N(C(=O)c1cc(F)ccc1Oc1nnc(Cl)c(N2CC3(CC(Oc4ccnc5c4CN(Cc4ccccc4)CC5)C3)C2)n1)C1CC2(CCCCN2C)C1 ; CC(C)N(C(=O)c1cc(F)ccc1Oc1nnc(Cl)c(N2CC3(CC(Oc4ccnc5c4CN(Cc4ccccc4)CC5)C3)C2)n1)C1CC2(CCCCN2C)C1]

A mixture of diastereomers of 2-((5-(6-((6-benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-(5-methyl-5-azaspiro[3.5]nonan-2-yl)benzamide and 2-((5-(6-((6-benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-(5-methyl-5-azaspiro[3.5]nonan-2-yl)benzamide was purified by chiral Prep SFC (Method AD) to obtain the major diastereomers.

Isomer 1. (D1)-2-((5-(6-((6-Benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-(5-methyl-5-azaspiro[3.5]nonan-2-yl)benzamide as a solid.

Yield: 16.94%; ¹H NMR (400 MHz, DMSO-d₆) δ 8.25-8.16 (m, 1H), 7.51-7.14 (m, 8H), 6.72-6.61 (m, 1H), 4.83-4.56 (m, 3H), 4.35-4.10 (m, 2H), 3.93-3.52 (m, 4H), 3.47 (s, 2H), 2.88-2.64 (m, 7H), 2.36-2.19 (m, 7H), 1.99-1.53 (m, 4H), 1.51-1.19 (m, 8H), 1.15-0.62 (m, 3H); LCMS (Method A): Rt=2.55-2.79 min, m/z=781.1 [M+H]⁺, HPLC (Method C): Rt=11.45 min, 98.16%; Chiral SFC (Method AAU): Rt=4.76 min, 100%.

Isomer 2. (D2)-2-((5-(6-((6-Benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-(5-methyl-5-azaspiro[3.5]nonan-2-yl)benzamide as a solid.

Yield: 9.87%; ¹H NMR (400 MHz, DMSO-d₆) δ 8.24-8.16 (m, 1H), 7.13 (s, 8H), 6.72-6.60 (m, 1H), 4.80-4.56 (m, 3H), 4.30-4.14 (m, 2H), 3.73-3.56 (m, 4H), 3.47 (br s, 2H), 2.87-2.66 (m, 7H), 2.32-2.12 (m, 4H), 1.99-1.54 (m, 3H), 1.52-1.20 (m, 8H), 1.14-0.66 (m, 3H), four protons merged with solvent peaks; LCMS (Method H): Rt=1.34 min, m/z=781.3 [M+H]⁺, HPLC (Method G): Rt=7.82 min, 97.62%; Chiral SFC (Method AAS): Rt=1.07 min, 98.26%.

Note: Minor diastereomers were not isolated.

### Step 3. (D1)-5-Fluoro-N-isopropyl-N-(5-methyl-5-azaspiro[3.5]nonan-2-yl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide

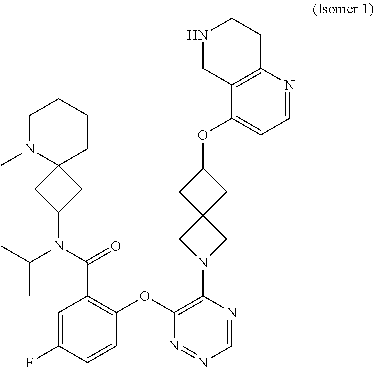 [SMILES: CC(C)N(C(=O)c1cc(F)ccc1Oc1nncnc1N1CC2(CC(Oc3ccnc4c3CNCC4)C2)C1)C1CC2(CCCCN2C)C1]

This compound was synthesized following the procedure described for the synthesis of Example 59, step 5, starting with 1 equivalent of (D1)-2-((5-(6-((6-benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-(5-methyl-5-azaspiro[3.5]nonan-2-yl)benzamide, except that 0.734 equivalent of Pd—C was used. After completion, the reaction was filtered through a Celite® pad, and the pad was washed with MeOH. The filtrate was concentrated under reduced pressure. The crude product was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure to afford (D1)-5-fluoro-N-isopropyl-N-(5-methyl-5-azaspiro[3.5]nonan-2-yl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide as a solid.

Yield: 55.3%; ¹H NMR (400 MHz, DMSO-d₆): δ 8.46 (s, 1H), 8.17 (br d, J=5.0 Hz, 1H), 7.51-7.13 (m, 3H), 6.71-6.58 (m, 1H), 4.87-4.50 (m, 3H), 4.30-4.06 (m, 2H), 3.99-3.84 (m, 1H), 3.70 (s, 2H), 3.67-3.52 (m, 2H), 2.99-2.91 (m, 2H), 2.88-2.74 (m, 3H), 2.72-2.64 (m, 2H), 2.33-2.14 (m, 6H), 2.02-1.61 (m, 4H), 1.54-1.21 (m, 8H), 1.12-0.57 (m, 3H), one proton merged with solvent peaks; LCMS (Method H): Rt=0.97 min, m/z=657.3 [M+H]⁺,HPLC (Method G): Rt=6.40 min, 98.18%; Chiral SFC (Method P): Rt=5.51 min, 100%.

### Step 4. (D1)-5-Fluoro-N-isopropyl-2-((5-(6-((6-methyl-5,6,7,8-tetrahydro-1,6-naphthyridin-4-yl)oxy)-2-azaspiro[3.3]heptan-2-yl)-1,2,4-triazin-6-yl)oxy)-N-(5-methyl-5-azaspiro[3.5]nonan-2-yl)benzamide

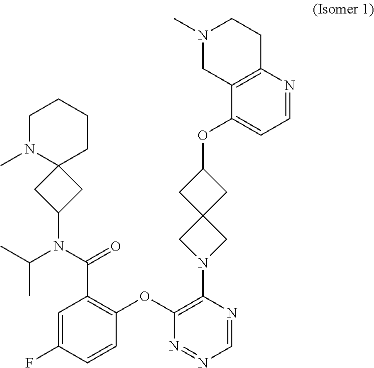 [SMILES: CC(C)N(C(=O)c1cc(F)ccc1Oc1nncnc1N1CC2(CC(Oc3ccnc4c3CN(C)CC4)C2)C1)C1CC2(CCCCN2C)C1]

To a stirred solution of (D1)-5-fluoro-N-isopropyl-N-((5-methyl-5-azaspiro[3.5]nonan-2-yl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide (150 mg, 0.228 mmol) in MeOH (3 mL), formaldehyde (0.090 mL, 1.142 mmol) and AcOH (3.31 μL, 0.057 mmol) were added at 0° C. The reaction was stirred at RT for 10 min. To this reaction mixture, sodium triacetoxyborohydride (72.6 mg, 0.343 mmol) was added at 0° C., and the reaction was stirred at RT for 6 h. The reaction progress was monitored by TLC (10% MeOH in DCM) and LCMS. After completion, the reaction was concentrated under reduced pressure, the residue was diluted with aqueous NaHCO₃ solution (10 mL), and the mixture was extracted with 10% MeOH in DCM (3×45 mL). The combined organic layer was dried over anhydrous sodium sulfate and filtered, and the filtrate was concentrated under reduced pressure. The crude (180 mg) was purified by reverse phase chromatography using a SiliCycle SiliaSep C18 column (40 g) on Biotage isolera one (Mobile phase A: 10 mM NH₄HCO₃ in water, B: ACN) to obtain (D1)-5-fluoro-N-isopropyl-2-((5-(6-((6-methyl-5,6,7,8-tetrahydro-1,6-naphthyridin-4-yl)oxy)-2-azaspiro[3.3]heptan-2-yl)-1,2,4-triazin-6-yl)oxy)-N-(5-methyl-5-azaspiro[3.5]nonan-2-yl)benzamide (101.54 mg, 65.8% yield) as a solid. ¹H NMR (400 MHz, DMSO-d₆) δ 8.47 (s, 1H), 8.20 (br d, J=4.9 Hz, 1H), 7.50-7.13 (m, 3H), 6.73-6.59 (m, 1H), 4.87-4.52 (m, 3H), 4.32-4.08 (m, 2H), 3.99-3.86 (m, 1H), 3.71-3.52 (m, 2H), 3.38 (br s, 2H), 2.90-2.78 (m, 4H), 2.67-2.60 (m, J=5.0 Hz, 2H), 2.41-2.34 (m, 4H), 2.29-2.15 (m, 5H), 1.99-1.63 (m, 4H), 1.55-1.18 (m, 8H), 1.11-0.59 (m, 3H), one proton merged with solvent peaks; LCMS (Method H): Rt=1.00 min, m/z=671.4 [M+H]⁺; HPLC (Method G): Rt=6.44 min, 99.80%; Chiral SFC (Method P). Rt=5.00 min, 94.95%.

### Step 5. (D2)-5-Fluoro-N-isopropyl-N-(5-methyl-5-azaspiro[3.5]nonan-2-yl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide

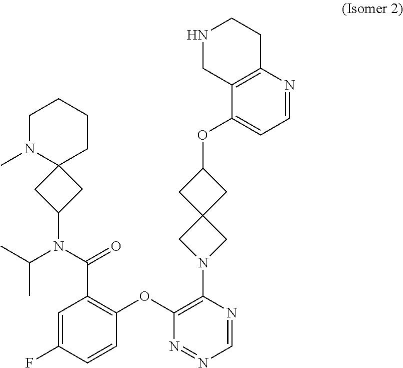 [SMILES: CC(C)N(C(=O)c1cc(F)ccc1Oc1nncnc1N1CC2(CC(Oc3ccnc4c3CNCC4)C2)C1)C1CC2(CCCCN2C)C1]

This compound was synthesized following the procedure described for the synthesis of Example 59, step 5, starting with 1 equivalent of (D2)-2-((5-(6-((6-benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-(5-methyl-5-azaspiro[3.5]nonan-2-yl)benzamide, except that 0.734 equivalent of Pd—C was used. After completion, the reaction was filtered through a Celite® pad, and the pad was washed with MeOH. The filtrate was concentrated under reduced pressure. The crude product was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure to afford crude (D2)-5-fluoro-N-isopropyl-N-(5-methyl-5-azaspiro[3.5]nonan-2-yl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide as a solid.

Yield: 48.8%; LCMS (Method H): Rt=1.00 min, m/z=657.3 [M+H]⁺, 64.88%.

### Step 6. (D2)-5-Fluoro-N-isopropyl-2-((5-(6-((6-methyl-5,6,7,8-tetrahydro-1,6-naphthyridin-4-yl)oxy)-2-azaspiro[3.3]heptan-2-yl)-1,2,4-triazin-6-yl)oxy)-N-(5-methyl-5-azaspiro[3.5]nonan-2-yl)benzamide

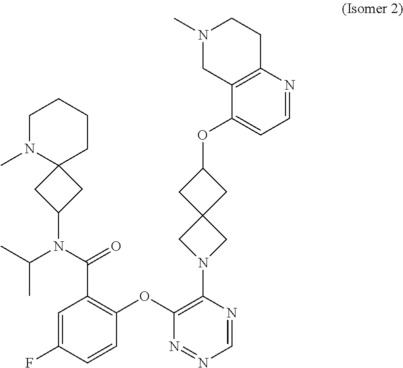 [SMILES: CC(C)N(C(=O)c1cc(F)ccc1Oc1nncnc1N1CC2(CC(Oc3ccnc4c3CN(C)CC4)C2)C1)C1CC2(CCCCN2C)C1]

To a stirred solution of (D2)-5-fluoro-N-isopropyl-N-(5-methyl-5-azaspiro[3.5]nonan-2-yl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide (120 mg, 0.183 mmol) in MeOH (3 mL), formaldehyde (0.068 mL, 0.914 mmol) and AcOH (5.23 μL, 0.091 mmol) were added at 0° C. The reaction was stirred at RT for 1 h. To this reaction mixture, sodium triacetoxyborohydride (58.1 mg, 0.274 mmol) was added at 0° C., and the reaction was stirred at RT for 4 h. The reaction progress was monitored by LCMS. After completion, the reaction was concentrated under reduced pressure. The residue was diluted with saturated sodium bicarbonate solution (5 mL) and the mixture was extracted with 10% MeOH in DCM (2×10 mL). The combined organic layer was dried over sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain (D2)-5-fluoro-N-isopropyl-2-((5-(6-((6-methyl-5,6,7,8-tetrahydro-1,6-naphthyridin-4-yl)oxy)-2-azaspiro[3.3]heptan-2-yl)-1,2,4-triazin-6-yl)oxy)-N-(5-methyl-5-azaspiro[3.5]nonan-2-yl)benzamide (55 mg, 44.7% yield). ¹H NMR (400 MHz, DMSO-d₆) δ 8.47 (br s, 1H), 8.20 (d, J=5.5 Hz, 1H), 7.49-7.13 (m, 3H), 6.74-6.60 (m, 1H), 4.86-4.50 (m, 3H), 4.33-4.10 (m, 2H), 3.75-3.47 (m, 3H), 3.37 (br s, 2H), 3.12-2.73 (m, 5H), 2.67-2.59 (m, 2H), 2.41-2.02 (m, 10H), 1.94-1.51 (m, 3H), 1.50-1.14 (m, 8H), 1.11-0.58 (m, 3H); LCMS (Method H): Rt=1.02 min, m/z=671.4 [M+H]⁺; HPLC (Method G): Rt=6.52 min, 99.50%; Chiral SFC (Method A): Rt=2.64 min, 94.15%.

### Example 208. 5-Fluoro-N-isopropyl-N-((2s,4r)-5-methyl-5-azaspiro[3.4]octan-2-yl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide (Compound No. 208)

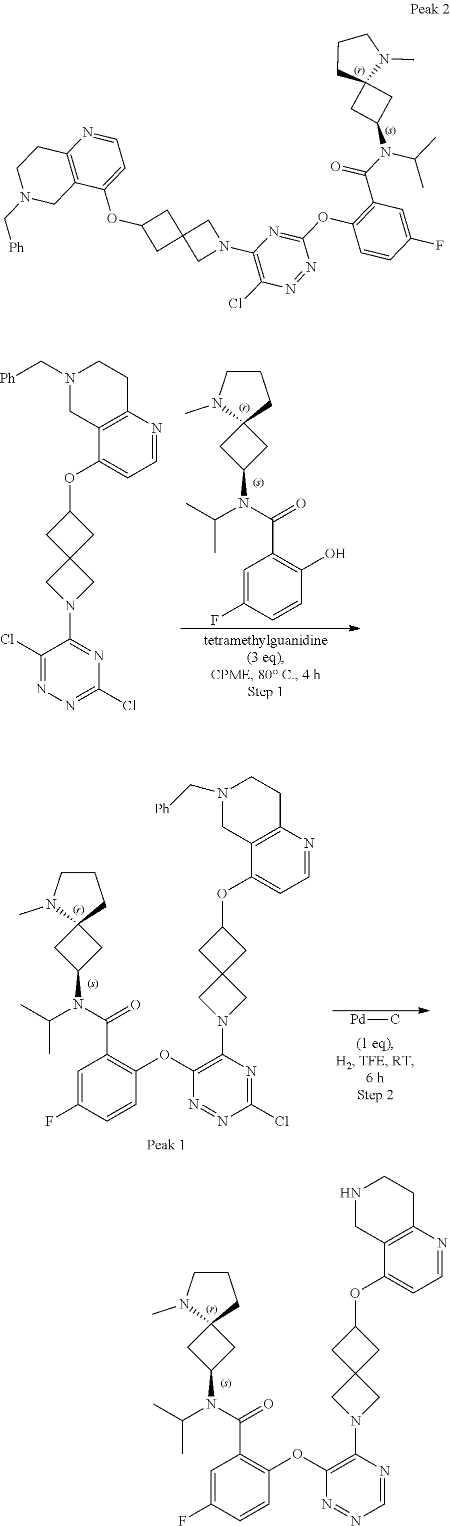

### Step 1. 2-((5-(6-((6-Benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-((2s,4r)-5-methyl-5-azaspiro[3.4]octan-2-yl)benzamide and 2-((5-(6-((6-benzyl-5,6,7,8-tetrahydro-1,6-naphthyridin-4-yl)oxy)-2-azaspiro[3.3]heptan-2-yl)-6-chloro-1,2,4-triazin-3-yl)oxy)-5-fluoro-N-isopropyl-N-((2s,4r)-5-methyl-5-azaspiro[3.4]octan-2-yl)benzamide

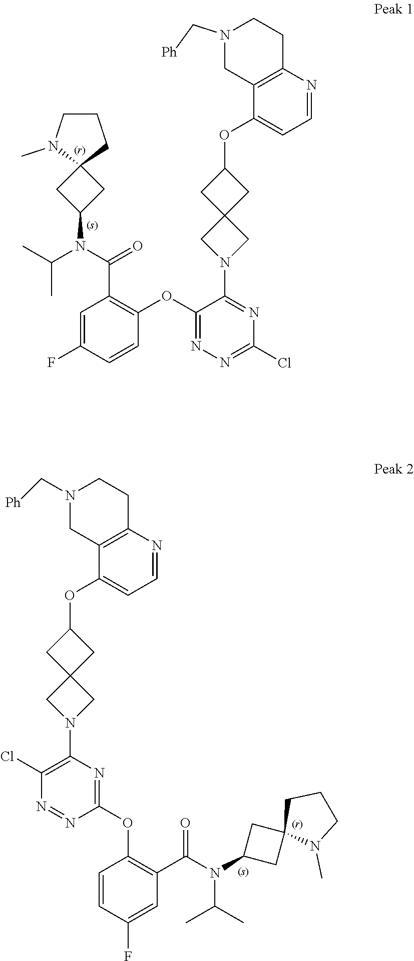 [SMILES: CC(C)N(C(=O)c1cc(F)ccc1Oc1nnc(Cl)nc1N1CC2(CC(Oc3ccnc4c3CN(Cc3ccccc3)CC4)C2)C1)[C@H]1C[C@@]2(CCCN2C)C1 ; CC(C)N(C(=O)c1cc(F)ccc1Oc1nnc(Cl)c(N2CC3(CC(Oc4ccnc5c4CN(Cc4ccccc4)CC5)C3)C2)n1)[C@H]1C[C@@]2(CCCN2C)C1]

This compound was synthesized following the procedure described for the synthesis of Example 164, step 2, starting with 1 equivalent of 6-benzyl-4-((2-(3,6-dichloro-1,2,4-triazin-5-yl)-2-azaspiro[3.3]heptan-6-yl)oxy)-5,6,7,8-tetrahydro-1,6-naphthyridine and 5-fluoro-2-hydroxy-N-isopropyl-N-((2s,4r)-5-methyl-5-azaspiro[3.4]octan-2-yl)benzamide and 1 equivalent of 5-fluoro-2-hydroxy-N-isopropyl-N-((2s,4r)-5-methyl-5-azaspiro[3.4]octan-2-yl)benzamide. After completion, the reaction was diluted with water and the mixture was extracted with EtOAc. The combined organic layer was washed with brine, dried over anhydrous sodium sulfate and filtered, and the filtrate was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN), then by achiral Prep SFC (Method G). The pure fractions were concentrated under reduced pressure to obtain the major isomer 2-((5-(6-((6-benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-((2s,4r)-5-methyl-5-azaspiro[3.4]octan-2-yl)benzamide as solid.

Yield: 27.54%; LCMS (Method A): Rt=2.31 min, m/z=767.2 [M+H]⁺; HPLC (Method C): Rt=10.94 min, 95.48%.

Note: The other regioisomer was not isolated.

### Step 2. 5-Fluoro-N-isopropyl-N-((2s,4r)-5-methyl-5-azaspiro[3.4]octan-2-yl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide

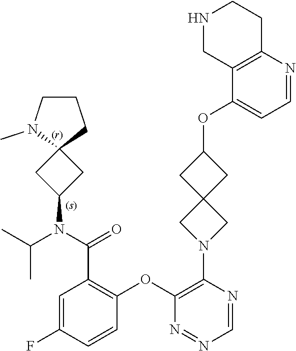 [SMILES: CC(C)N(C(=O)c1cc(F)ccc1Oc1nncnc1N1CC2(CC(Oc3ccnc4c3CNCC4)C2)C1)[C@H]1C[C@@]2(CCCN2C)C1]

This compound was synthesized following the procedure described for the synthesis of Example 59, step 5, starting with 1 equivalent of 2-((5-(6-((6-benzyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-((2s,4r)-5-methyl-5-azaspiro[3.4]octan-2-yl)benzamide, except that 1 equivalent of Pd—C was used. After completion, the reaction was filtered through a Celite® pad, the pad was washed with TFE, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain 5-fluoro-N-isopropyl-N-((2s,4r)-5-methyl-5-azaspiro[3.4]octan-2-yl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide) as a solid.

Yield: 48.5%; ¹H NMR (400 MHz, DMSO-d₆): δ 8.46 (s, 1H), 8.17 (br d, J=4.6 Hz, 1H), 7.50-7.10 (m, 3H), 6.73-6.55 (m, 1H), 4.83-4.50 (m, 3H), 4.00 (d, J=2.1 Hz, 3H), 3.71 (s, 2H), 3.68-3.50 (m, 2H), 2.96 (br t, J=5.5 Hz, 2H), 2.87-2.75 (m, 2H), 2.72-2.65 (m, 2H), 2.41-2.15 (m, 6H), 1.95 (s, 3H), 1.88-1.41 (m, 7H), 1.29-1.16 (m, 2H), 1.12-0.52 (m, 3H); LCMS (Method H): Rt=1.02 min, m/z=643.3 [M+H]⁺; HPLC (Method G): Rt=6.39 min, 99.22%; Chiral SFC (Method AAJ): Rt=1.20 min, 100%.

### Example 209. (D1)-2-((5-(6-((5-Ethyl-5,6,7,8-tetrahydro-1,6-naphthyridin-4-yl)oxy)-2-azaspiro[3.3]heptan-2-yl)-1,2,4-triazin-6-yl)oxy)-5-fluoro-N-isopropyl-N-((1r,3r)-3-methyl-3-(pyrrolidin-1-yl)cyclobutyl)benzamide (Compound No. 209)

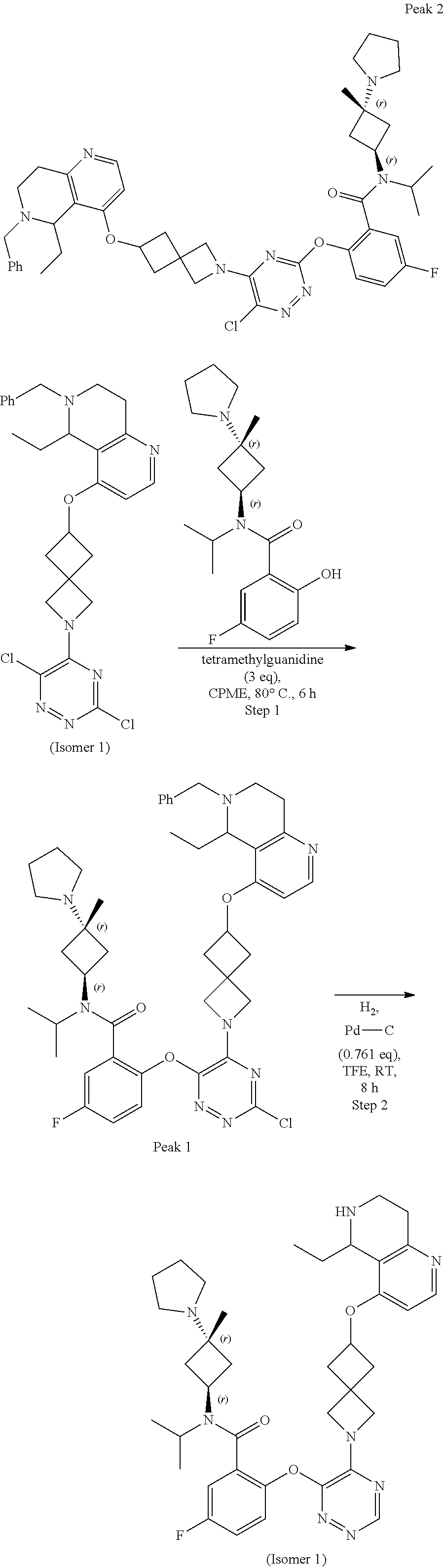

### Step 1. (D1)-2-((5-(6-((6-Benzyl-5-ethyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-((1r,3r)-3-methyl-3-(pyrrolidin-1-yl)cyclobutyl)benzamide and (D)-2-((5-(6-((6-benzyl-5-ethyl-5,6,7,8-tetrahydro-1,6-naphthyridin-4-yl)oxy)-2-azaspiro[3.3]heptan-2-yl)-6-chloro-1,2,4-triazin-3-yl)oxy)-5-fluoro-N-isopropyl-N-((1r,3r)-3-methyl-3-(pyrrolidin-1-yl)cyclobutyl)benzamide

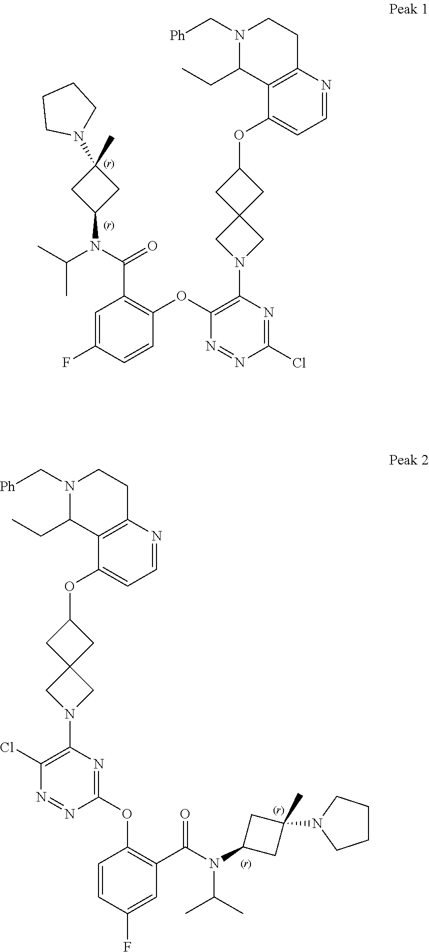 [SMILES: CCC1c2c(OC3CC4(C3)CN(c3nc(Cl)nnc3Oc3ccc(F)cc3C(=O)N(C(C)C)[C@H]3C[C@](C)(N5CCCC5)C3)C4)ccnc2CCN1Cc1ccccc1 ; CCC1c2c(OC3CC4(C3)CN(c3nc(Oc5ccc(F)cc5C(=O)N(C(C)C)[C@H]5C[C@](C)(N6CCCC6)C5)nnc3Cl)C4)ccnc2CCN1Cc1ccccc1]

This compound was synthesized following the procedure described for the synthesis of Example 164, Step 2, starting with 1 equivalent of (E1)-6-benzyl-4-((2-(3,6-dichloro-1,2,4-triazin-5-yl)-2-azaspiro[3.3]heptan-6-yl)oxy)-5-ethyl-5,6,7,8-tetrahydro-1,6-naphthyridine and 1 equivalent of 5-fluoro-2-hydroxy-N-isopropyl-N-((1r,3r)-3-methyl-3-(pyrrolidin-1-yl)cyclobutyl)benzamide. After completion, the reaction was quenched with water and the mixture was extracted with EtOAc. The combined organic layer was dried over sodium sulfate and filtered, and the filtrate was concentrated under reduced pressure. The crude was further purified by reverse phase chromatography using a SiliCycle SiliaSep C18 column (120 g) on Biotage isolera one (Mobile phase A: 10 mM NH₄HCO₃ in water, B: ACN), then by achiral Prep SFC (Method H). The pure fractions were concentrated under reduced pressure to obtain both regioisomers.

Peak 1. (D1)-2-((5-(6-((6-Benzyl-5-ethyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-((1r,3r)-3-methyl-3-(pyrrolidin-1-yl)cyclobutyl)benzamide as a solid.

Yield: 39.2%; ¹H NMR (400 MHz, DMSO-d₆): δ 8.21 (br s, 1H), 7.50-7.11 (m, 8H), 6.69 (br s, 1H), 4.82-4.58 (m, 3H), 4.33-3.93 (m, 3H), 3.82-3.47 (m, 5H), 2.97-2.75 (m, 5H), 2.39-2.13 (m, 5H), 1.86-1.36 (m, 10H), 1.30-0.60 (m, 12H), one proton merged with solvent peaks; LCMS (Method B): Rt=1.40 min, m/z=809.5 [M+H]⁺; HPLC (Method G): Rt=7.11 min, 99.17%.

Peak 2. (D1)-2-((5-(6-((6-Benzyl-5-ethyl-5,6,7,8-tetrahydro-1,6-naphthyridin-4-yl)oxy)-2-azaspiro[3.3]heptan-2-yl)-6-chloro-1,2,4-triazin-3-yl)oxy)-5-fluoro-N-isopropyl-N-((1r,3r)-3-methyl-3-(pyrrolidin-1-yl)cyclobutyl)benzamide as a solid.

Yield: 8.72%; LCMS (Method B): Rt=1.35 min, m/z=809.3 [M+H]⁺; HPLC (Method G): Rt=7.19 min, 72.45%.

### Step 2. (D1)-2-((5-(6-((5-Ethyl-5,6,7,8-tetrahydro-1,6-naphthyridin-4-yl)oxy)-2-azaspiro[3.3]heptan-2-yl)-1,2,4-triazin-6-yl)oxy)-5-fluoro-N-isopropyl-N-((1r,3r)-3-methyl-3-(pyrrolidin-1-yl)cyclobutyl)benzamide

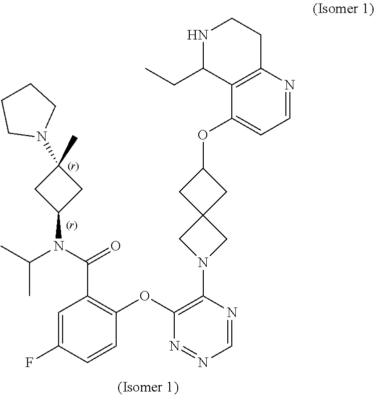 [SMILES: CCC1NCCc2nccc(OC3CC4(C3)CN(c3ncnnc3Oc3ccc(F)cc3C(=O)N(C(C)C)[C@H]3C[C@](C)(N5CCCC5)C3)C4)c21]

This compound was synthesized following the procedure described for the synthesis of Example 59, step 5, starting with 1 equivalent of (D1)-2-((5-(6-((6-benzyl-5-ethyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-((1r,3r)-3-methyl-3-(pyrrolidin-1-yl)cyclobutyl)benzamide, except that 0.761 equivalent of Pd—C was used. After completion, the reaction was filtered through a Celite® pad, the pad was washed with MeOH, and the filtrate was concentrated under reduced pressure. The crude product was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford (D1)-2-((5-(6-((5-ethyl-5,6,7,8-tetrahydro-1,6-naphthyridin-4-yl)oxy)-2-azaspiro[3.3]heptan-2-yl)-1,2,4-triazin-6-yl)oxy)-5-fluoro-N-isopropyl-N-((1r,3r)-3-methyl-3-(pyrrolidin-1-yl)cyclobutyl)benzamide as a solid.

Yield: 51.4%; ¹H NMR (400 MHz, DMSO-d₆): δ 8.46 (s, 1H), 8.16 (br d, J=5.1 Hz, 1H), 7.49-7.11 (m, 3H), 6.64 (br s, 1H), 4.83-4.73 (m, 1H), 4.71-4.50 (m, 2H), 4.29-3.95 (m, 2H), 3.81-3.50 (m, 3H), 3.06-2.76 (m, 5H), 2.66-2.58 (m, 2H), 2.30-2.08 (m, 4H), 1.84-1.34 (m, 10H), 1.31-0.53 (m, 1H), three protons merged with solvent peaks; LCMS (Method H): Rt=1.04 min, m/z=685.3 [M+H]⁺; HPLC (Method G): Rt=6.54 min, 99.39%; Chiral SFC (Method J): Rt=5.27 min, 100%.

### Example 210. (D1)-2-((5-(6-((5-Ethyl-5,6,7,8-tetrahydro-1,6-naphthyridin-4-yl)oxy)-2-azaspiro[3.3]heptan-2-yl)-1,2,4-triazin-6-yl)oxy)-5-fluoro-N-isopropyl-N-((2s,4r)-5-methyl-5-azaspiro[3.4]octan-2-yl)benzamide (Compound No. 210)

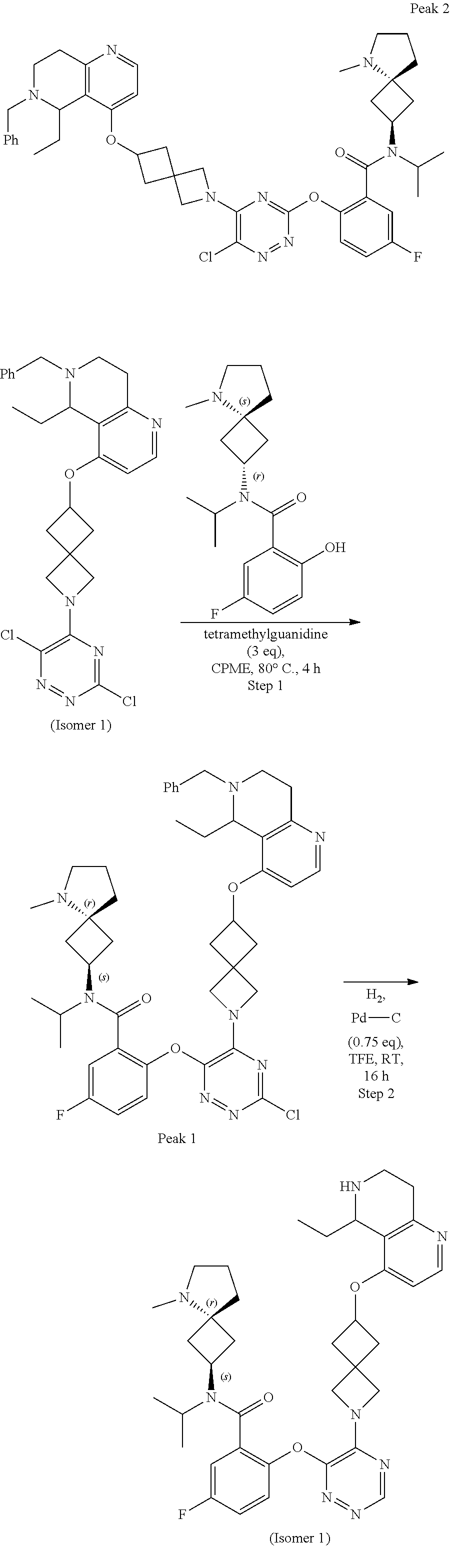

### Step 1. 2-((5-(6-((6-Benzyl-5-ethyl-5,6,7,8-tetrahydro-1,6-naphthyridin-4-yl)oxy)-2-azaspiro[3.3]heptan-2-yl)-3-chloro-1,2,4-triazin-6-yl)ox)-5-fluoro-N-isopropyl-N-((2s,4r)-5-methyl-5-azaspiro[3.4]octan-2-yl)benzamide and 2-((5-(6-((6-benzyl-5-ethyl-5,6,7,8-tetrahydro-1,6-naphthyridin-4-yl)oxy)-2-azaspiro[3.3]heptan-2-yl)-6-chloro-1,2,4-triazin-3-yl)oxy)-5-fluoro-N-isopropyl-N-((2s,4r)-5-methyl-5-azaspiro[3.4]octan-2-yl)benzamide

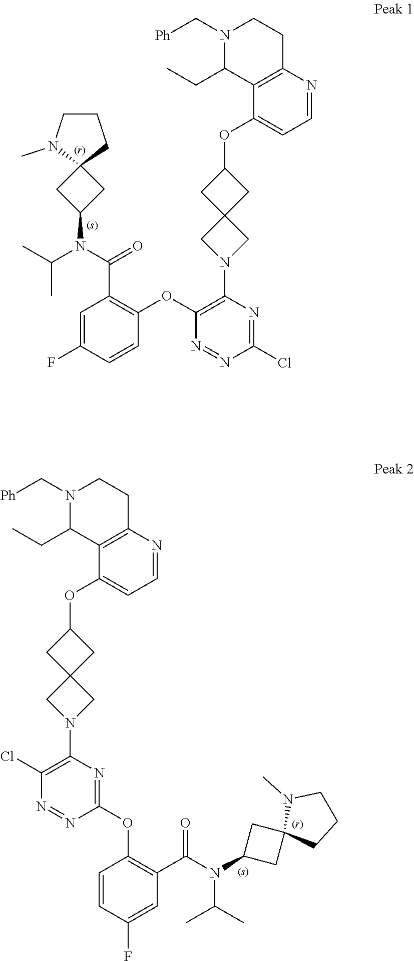 [SMILES: CCC1c2c(OC3CC4(C3)CN(c3nc(Cl)nnc3Oc3ccc(F)cc3C(=O)N(C(C)C)[C@H]3C[C@@]5(CCCN5C)C3)C4)ccnc2CCN1Cc1ccccc1 ; CCC1c2c(OC3CC4(C3)CN(c3nc(Oc5ccc(F)cc5C(=O)N(C(C)C)[C@H]5C[C@@]6(CCCN6C)C5)nnc3Cl)C4)ccnc2CCN1Cc1ccccc1]

This compound was synthesized following the procedure described for the synthesis of Example 164, Step 2, starting with 1 equivalent of (E1)-6-benzyl-4-((2-(3,6-dichloro-1,2,4-triazin-5-yl)-2-azaspiro[3.3]heptan-6-yl)oxy)-5-ethyl-5,6,7,8-tetrahydro-1,6-naphthyridine and 1 equivalent of 5-fluoro-2-hydroxy-N-isopropyl-N-((2s,4r)-5-methyl-5-azaspiro[3.4]octan-2-yl)benzamide. After completion, the reaction was quenched with 10% NaHCO₃ solution and the mixture was extracted with 10% MeOH in DCM. The combined organic layer was dried over sodium sulfate and filtered, and the filtrate was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN), then by chiral Prep SFC (Method AC). The pure fractions were concentrated under reduced pressure to obtain the major regioisomer.

Peak 1. (D1)-2-((5-(6-((6-Benzyl-5-ethyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-((2s,4r)-5-methyl-5-azaspiro[3.4]octan-2-yl)benzamide as a solid.

Yield: 25.4%; ¹H NMR (400 MHz, DMSO-d₆): δ 8.26-8.18 (m, 1H), 7.51-7.14 (m, 8H), 6.73-6.61 (m, 1H), 4.83-4.54 (m, 3H), 4.32-4.15 (m, 2H), 3.94-3.48 (m, 6H), 3.24-3.13 (m, 1H), 2.99-2.72 (m, 5H), 2.42-2.15 (m, 6H), 2.02-1.77 (m, 4H), 1.74-1.20 (m, 8H), 1.12-0.57 (m, 6H), one proton merged with solvent peaks; LCMS (Method A): Rt=2.61 min, m/z=795.2 [M+H]⁺; HPLC (Method C): Rt=12.26 min, 98.33%.

Note: The other regioisomer was not isolated.

### Step 2. (D1)-2-((5-(6-((5-Ethyl-5,6,7,8-tetrahydro-1,6-naphthyridin-4-yl)oxy)-2-azaspiro[3.3]heptan-2-yl)-1,2,4-triazin-6-yl)oxy)-5-fluoro-N-isopropyl-N-((2s,4r)-5-methyl-5-azaspiro[3.4]octan-2-yl)benzamide

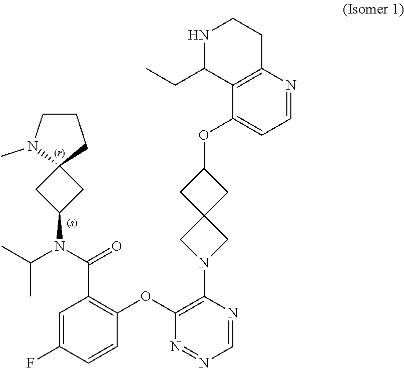 [SMILES: CCC1NCCc2nccc(OC3CC4(C3)CN(c3ncnnc3Oc3ccc(F)cc3C(=O)N(C(C)C)[C@H]3C[C@@]5(CCCN5C)C3)C4)c21]

This compound was synthesized following the procedure described for the synthesis of Example 59, step 5, starting with 1 equivalent of (D1)-2-((5-(6-((6-benzyl-5-ethyl-5,6,7,8-tetrahydro-1,6-naphthyridin-4-yl)oxy)-2-azaspiro[3.3]heptan-2-yl)-3-chloro-1,2,4-triazin-6-yl)oxy)-5-fluoro-N-isopropyl-N-((2s,4r)-5-methyl-5-azaspiro[3.4]octan-2-yl)benzamide, except that 0.75 equivalent of Pd—C was used. After completion, the reaction was filtered through a Celite® pad, the pad was washed with MeOH, and the filtrate was concentrated under reduced pressure. The crude product was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford (D1)-2-((5-(6-((5-ethyl-5,6,7,8-tetrahydro-1,6-naphthyridin-4-yl)oxy)-2-azaspiro[3.3]heptan-2-yl)-1,2,4-triazin-6-yl)oxy)-5-fluoro-N-isopropyl-N-((2s,4r)-5-methyl-5-azaspiro[3.4]octan-2-yl)benzamide as a solid.

Yield: 38.1%; ¹H NMR (400 MHz, DMSO-d₆): δ 8.46 (s, 1H), 8.16 (br d, J=5.0 Hz, 1H), 7.51-7.08 (m, 3H), 6.72-6.56 (m, 1H), 4.84-4.53 (m, 3H), 4.30-4.07 (m, 2H), 3.98-3.82 (m, 1H), 3.75 (br d, J=8.4 Hz, 1H), 3.70-3.48 (m, 2H), 3.07-2.75 (m, 5H), 2.73-2.56 (m, 2H), 2.43-2.16 (m, 7H), 2.04-1.75 (m, 4H), 1.69-1.38 (m, 6H), 1.30-0.52 (m, 7H), one proton merged with solvent peaks; LCMS (Method B): Rt=0.87 min, m/z=671.4 [M+H]⁺; HPLC (Method G): Rt=6.49 min, 99.84%; Chiral SFC (Method C): Rt=3.92 min, 100%.

### Example 211. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2r,4s)-5-methyl-5-azaspiro[3.5]nonan-2-yl)benzamide (Compound No. 211) (Method 1)

### Example 212. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-methyl-5-azaspiro[3.5]nonan-2-yl)benzamide (Compound No. 212) (Method 1)

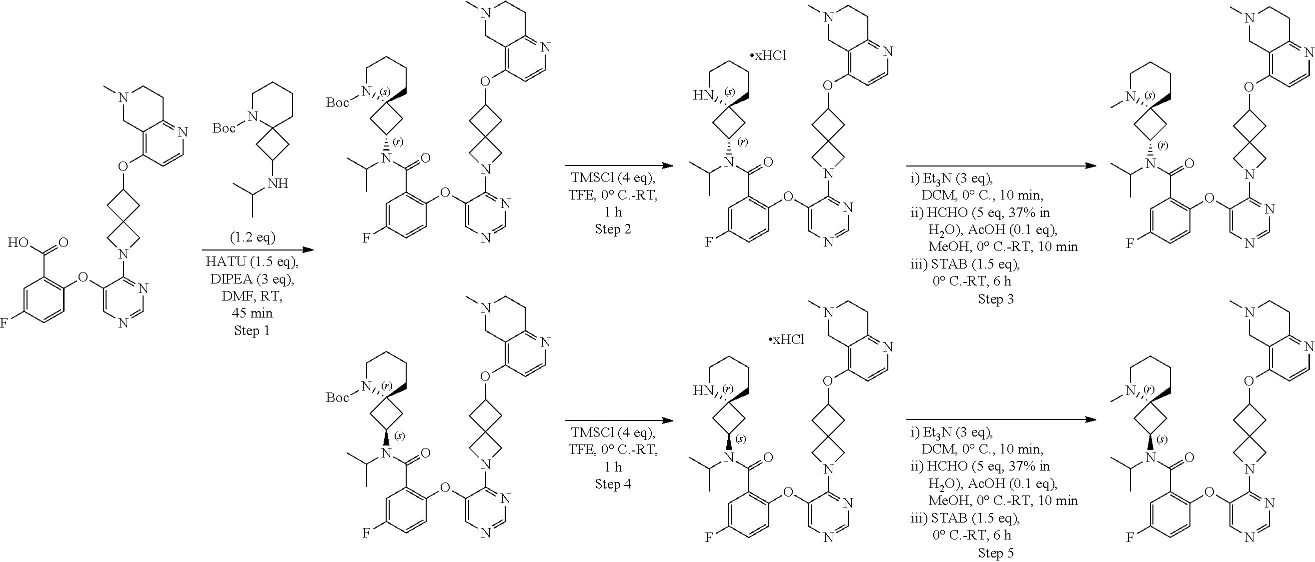

### Step 1. tert-Butyl(2r, 4s)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.5]nonane-5-carboxylate and tert-butyl(2s,4r)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.5]nonane-5-carboxylate

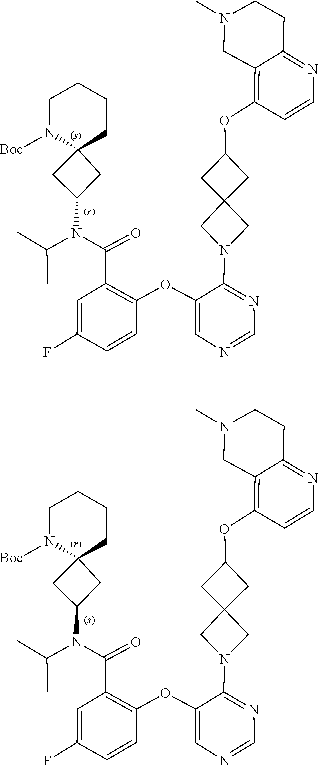 [SMILES: CC(C)N(C(=O)c1cc(F)ccc1Oc1cncnc1N1CC2(CC(Oc3ccnc4c3CN(C)CC4)C2)C1)[C@H]1C[C@]2(CCCCN2C(=O)OC(C)(C)C)C1 ; CC(C)N(C(=O)c1cc(F)ccc1Oc1cncnc1N1CC2(CC(Oc3ccnc4c3CN(C)CC4)C2)C1)[C@H]1C[C@@]2(CCCCN2C(=O)OC(C)(C)C)C1]

Step 1 was performed following the same procedure in two batches (820 and 750 mg).

In a 40 mL pressure vial, 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid (820 mg, 1.668 mmol) and tert-butyl 2-(isopropylamino)-5-azaspiro[3.5]nonane-5-carboxylate (565 mg, 2.002 mmol) were dissolved in DMF (10 mL). To this solution, DIPEA (0.874 mL, 5.00 mmol) and HATU (952 mg, 2.502 mmol) were added at 0° C. The reaction was stirred at RT for 45 min. The reaction progress was monitored by TLC (10% MeOH in DCM). After completion, the reaction mixtures from both batches were combined and quenched with ice water (50 mL), and the mixture was extracted with EtOAc (3×50 mL). The combined organic layer was washed with ice-cold brine (100 mL), dried over anhydrous sodium sulfate and filtered, and the filtrate was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN) to obtain a mixture of diastereomers, tert-butyl (2r,4s)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.5]nonane-5-carboxylate and tert-butyl(2s,4r)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.5]nonane-5-carboxylate (1.8 g). LCMS (Method A): Peak 1; Rt=2.18 min, 50.42%; Peak 2; Rt=2.25 min, 48.35%; m/z=757.3 [M+H]⁺; HPLC (Method C): Peak 1; Rt=10.80 min, 54.37%; Peak 2; Rt=11.19 min, 44.37%.

The mixture of diastereomers was further purified by Prep HPLC (Method E). The pure fractions were concentrated under reduced pressure to obtain both isomers.

Isomer 1. tert-Butyl(2r,4s)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.5]nonane-5-carboxylate (500 mg, 20.71% combined yield) as a solid. ¹H NMR (400 MHz, DMSO-d₆): δ 8.32-8.24 (m, 1H), 8.18 (br d, J=5.3 Hz, 1H), 7.78-7.66 (m, 1H), 7.32-7.01 (m, 3H), 6.66 (d, J=5.6 Hz, 1H), 4.75 (quin, J=6.7 Hz, 1H), 4.34-4.06 (m, 4H), 3.78-3.51 (m, 2H), 3.38-3.35 (m, 2H), 3.30-3.20 (m, 1H), 3.12 (br s, 2H), 2.85-2.75 (m, 4H), 2.65-2.60 (m, 2H), 2.36 (s, 3H), 2.30-1.93 (m, 5H), 1.69-1.48 (m, 3H), 1.45-1.21 (m, 15H), 1.17-0.98 (m, 3H); LCMS (Method A): Rt=2.16 min, m/z=756.8 [M+H]⁺; HPLC (Method C): Rt=10.82 min, 99.35%.

Isomer 2. tert-Butyl(2s,4r)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.5]nonane-5-carboxylate (450 mg, 18.64% combined yield) as a solid. ¹H NMR (400 MHz, DMSO-d₆): δ 8.31-8.25 (m, 1H), 8.17 (br d, J=5.5 Hz, 1H), 7.81-7.63 (m, 1H), 7.33-7.02 (m, 3H), 6.72-6.58 (m, 1H), 4.75 (quin, J=6.5 Hz, 1H), 4.36-4.07 (m, 4H), 4.07-3.83 (m, 1H), 3.69 (spt, J=6.5 Hz, 1H), 3.39-3.36 (m, 1H), 3.30-3.17 (m, 2H), 3.00-2.91 (m, 1H), 2.85-2.77 (m, 4H), 2.74-2.60 (m, 4H), 2.36 (s, 3H), 2.31-2.19 (m, 2H), 2.03-1.80 (m, 2H), 1.62-1.43 (m, 4H), 1.42-1.23 (m, 12H), 1.09-0.93 (m, 3H), two protons merged with solvent peaks; LCMS (Method A): Rt=2.25 min, m/z=756.8 [M+H]⁺; HPLC (Method C): Rt=11.19 min, 99.15%.

### Step 2. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2r,4s)-5-azaspiro[3.5]nonan-2-yl)benzamide xHCl

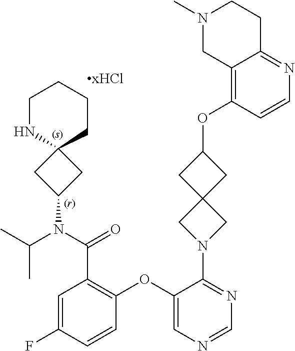 [SMILES: CC(C)N(C(=O)c1cc(F)ccc1Oc1cncnc1N1CC2(CC(Oc3ccnc4c3CN(C)CC4)C2)C1)[C@H]1C[C@]2(CCCCN2)C1]

This compound was synthesized following the procedure described for the synthesis of Example 20, step 3, starting with 1 equivalent of tert-butyl(2r,4s)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.5]nonane-5-carboxylate. After completion, the reaction was concentrated under reduced pressure, and the residue was triturated with EtOAc. The organic layer was decanted and the solid was dried under reduced pressure to obtain crude 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2r,4s)-5-azaspiro[3.5]nonan-2-yl)benzamide xHCl as a solid. This material was used without further purification

Yield: quantitative; LCMS (Method A): Rt=1.89 min, m/z=656.3 [M+H]⁺; HPLC (Method C): Rt=8.11 min, 98.56%.

### Step 3. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2r,4s)-5-methyl-5-azaspiro[3.5]nonan-2-yl)benzamide

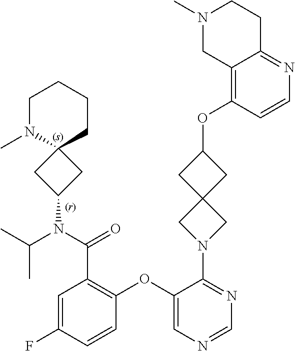

To a stirred solution of 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2r,4s)-5-azaspiro[3.5]nonan-2-yl)benzamide xHCl (700 mg, 1.011 mmol) in DCM (7 mL), TEA (0.423 mL, 3.03 mmol) was added at 0° C. The reaction was stirred at the same temperature for 10 min, then was concentrated under reduced pressure. The residue was dissolved in MeOH (7 mL), and formaldehyde (0.402 mL, 5.06 mmol, 37% in H₂O) and AcOH (5.84 μL, 0.101 mmol) were added at 0° C. The reaction was stirred at RT for 10 min, then STAB (321 mg, 1.517 mmol) was added at 0° C. The reaction was stirred at RT for 6 h. The reaction progress was monitored by TLC (10% MeOH in DCM). After completion, the reaction was concentrated under reduced pressure and the residue was quenched with aqueous NaHCO₃ solution (10 mL). The mixture was extracted with 10% MeOH in DCM (3×45 mL). The combined organic layer was dried over anhydrous sodium sulfate and filtered, and the filtrate was concentrated under reduced pressure to afford crude product (720 mg) as a gummy liquid. LCMS (Method A): Rt=1.84 min, m/z=670.3 [M+H]⁺, 94.07%.

This crude product (720 mg, 1.075 mmol) was dissolved in DCM (10 mL), and TEA (0.449 mL, 3.22 mmol) was added at 0° C. The reaction was stirred at RT for 10 min, then the mixture was concentrated under reduced pressure. The crude product was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure and lyophilized to obtain 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2r,4s)-5-methyl-5-azaspiro[3.5]nonan-2-yl)benzamide (262 mg, 36.1% yield) as a solid. ¹HNMR (400 MHz, DMSO-d₆): δ 8.33-8.22 (m, 1H), 8.18 (d, J=5.7 Hz, 1H), 7.87-7.59 (m, 1H), 7.32-7.01 (m, 3H), 6.70-6.60 (m, 1H), 4.75 (quin, J=6.6 Hz, 1H), 4.37-4.06 (m, 4H), 3.80-3.57 (m, 2H), 3.36 (s, 2H), 3.16-3.02 (m, 1H), 2.85-2.74 (m, 4H), 2.63 (br t, J=5.8 Hz, 2H), 2.36 (s, 3H), 2.31-2.01 (m, 6H), 1.87-1.55 (m, 3H), 1.52-0.94 (m, 11H), two protons merged with solve peaks; LCMS (Method H): Rt=1.06 min, m/z=670.3 [M+H]⁺; HPLC (Method G): Rt=6.39 min, 99.22%; Chiral SFC (Method K): Rt=2.88 min, 100%.

### Step 4. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-azaspiro[3.5]nonan-2-yl)benzamide

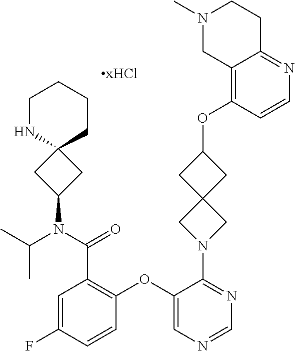 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCCN2)C1]

This compound was synthesized following the procedure described for the synthesis of Example 20, step 3, starting with 1 equivalent of tert-butyl(2s,4r)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.5]nonane-5-carboxylate. After completion, the reaction was concentrated under reduced pressure, and the residue was triturated with EtOAc. The organic layer was decanted and the solid was dried under reduced pressure to obtain crude 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-azaspiro[3.5]nonan-2-yl)benzamide xHCl as a solid. This material was used without further purification.

Yield: quantitative; LCMS (Method H): Rt=1.04 min, m/z=656.3 [M+H]⁺; HPLC (Method C): Rt=7.94 min, 97.41%.

### Step 5. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-methyl-5-azaspiro[3.5]nonan-2-yl)benzamide

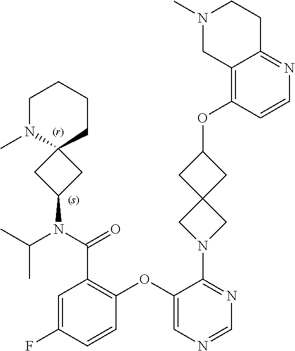 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCCN2C)C1]

To a stirred solution of 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-azaspiro[3.5]nonan-2-yl)benzamide xHCl (600 mg, 0.867 mmol) in DCM (6 mL), TEA (0.362 mL, 2.60 mmol) was added at 0° C. The reaction was stirred at the same temperature for 10 min, then was concentrated under reduced pressure. The residue was dissolved in MeOH (6 mL), and formaldehyde (0.344 mL, 4.33 mmol, 37% in H₂O) and AcOH (4.96 μL, 0.087 mmol) were added at 0° C. The reaction was stirred at RT for 10 min, then STAB (276 mg, 1.300 mmol) was added at 0° C. The reaction was stirred at RT for 6 h. The reaction progress was monitored by TLC (10% MeOH in DCM). After completion, the reaction was concentrated under reduced pressure and the residue was quenched with aqueous NaHCO₃ solution (10 mL). The mixture was extracted with 10% MeOH in DCM (3×45 mL). The combined organic layer was dried over anhydrous sodium sulfate and filtered, and the filtrate was concentrated under reduced pressure to afford crude product (600 mg) as a gummy liquid. LCMS (Method A): Rt=1.83 min, m/z=670.3 [M+H]⁺, 98.00%.

This crude product (600 mg, 0.896 mmol) was dissolved in DCM (10 mL), and TEA (0.375 mL, 2.69 mmol) was added at 0° C. The reaction was stirred at RT for 10 min, then the mixture was concentrated under reduced pressure. The crude product was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure and lyophilized to obtain 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-methyl-5-azaspiro[3.5]nonan-2-yl)benzamide (243 mg, 40.3% yield) as a solid. ¹HNMR (400 MHz, DMSO-d₆): δ 8.32-8.23 (m, 1H), 8.18 (br d, J=5.5 Hz, 1H), 7.80-7.55 (m, 1H), 7.34-7.06 (m, 3H), 6.71-6.58 (m, 1H), 4.75 (quin, J=6.6 Hz, 1H), 4.36-4.06 (m, 4H), 3.99-3.60 (m, 2H), 3.36 (s, 2H), 2.85-2.73 (m, 4H), 2.66-2.60 (m, 2H), 2.36 (s, 3H), 2.31-2.19 (m, 6H), 2.05-1.65 (m, 3H), 1.52-0.88 (m, 11H), three protons merged with solvent peaks; LCMS (Method H): Rt=1.03 min, m/z=670.3 [M+H]⁺; HPLC (Method C): Rt=8.46 min, 99.61%; Chiral SFC (Method H): Rt=4.83 min, 99.28%.

### Example 212. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-methyl-5-azaspiro[3.5]nonan-2-yl)benzamide (Compound No. 212) (Method 2)

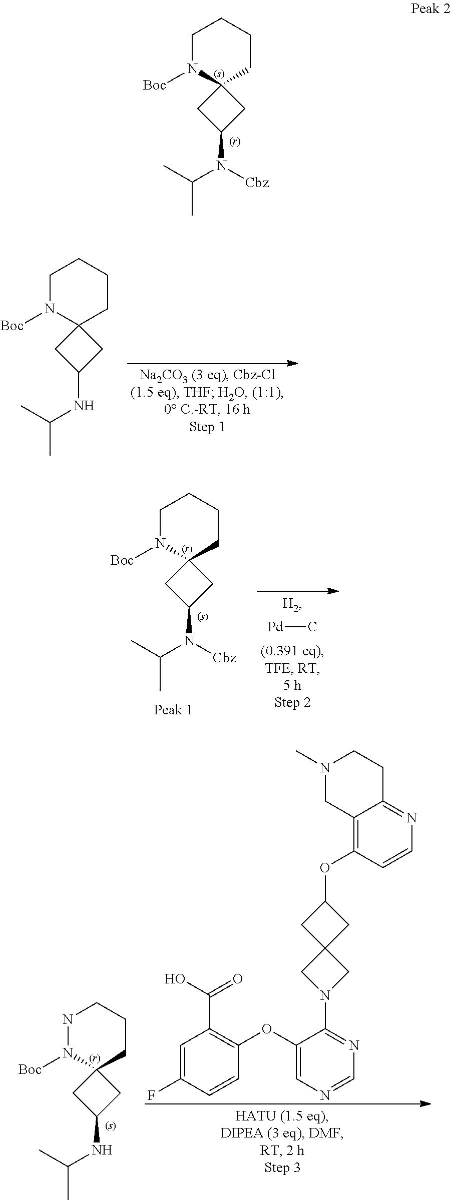

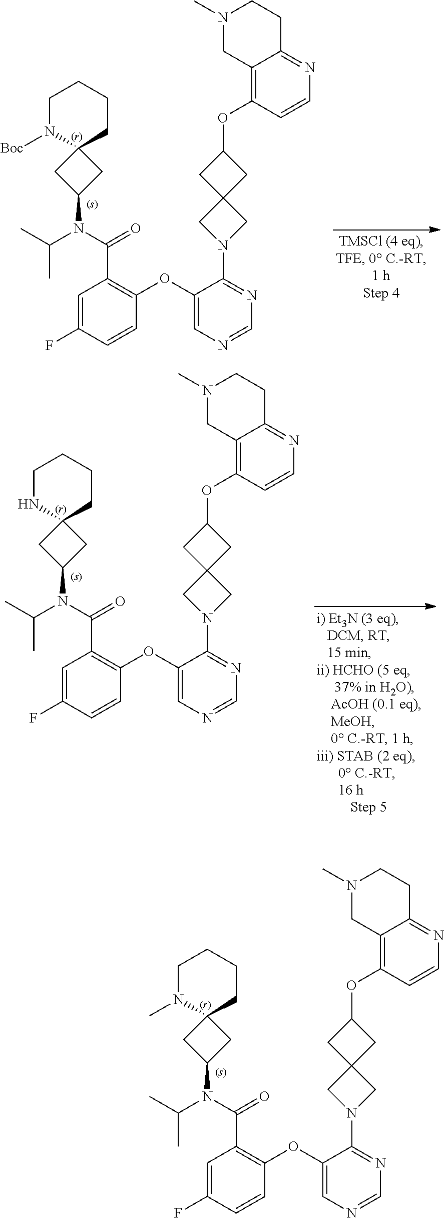

### Step 1. tert-Butyl(2s,4r)-2-(((benzyloxy)carbonyl)(isopropyl)amino)-5-azaspiro[3.5]nonane-5-carboxylate and tert-butyl(2r,4s)-2-(((benzyloxy)carbonyl)(isopropyl)amino)-5-azaspiro[3.5]nonane-5-carboxylate

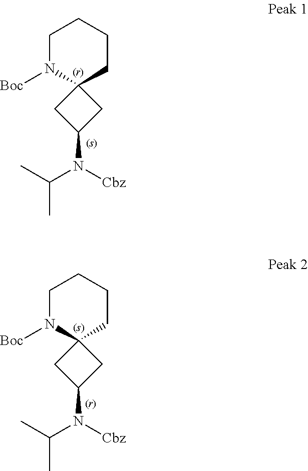 [SMILES: CC(C)N(C(=O)OCc1ccccc1)[C@H]1C[C@@]2(CCCCN2C(=O)OC(C)(C)C)C1 ; CC(C)N(C(=O)OCc1ccccc1)[C@H]1C[C@]2(CCCCN2C(=O)OC(C)(C)C)C1]

To a stirred solution of tert-butyl 2-(isopropylamino)-5-azaspiro[3.5]nonane-5-carboxylate (200 mg, 0.708 mmol) in 1:1 THF:H₂O (2 mL:2 mL), sodium bicarbonate (178 mg, 2.124 mmol) and Cbz-Cl (0.152 mL, 1.062 mmol) were added at 0° C. The reaction was stirred at RT for 16 h under a nitrogen atmosphere. The reaction progress was monitored by TLC (30% EtOAc in hexane). After completion, the reaction was cooled to 0° C.-5° C. and quenched with sodium bicarbonate solution (10 mL), and the mixture was extracted with EtOAc (3×10 mL). The combined organic layer was washed with brine solution (30 mL). The combined organic layer was dried over sodium sulfate and filtered, and the filtrate was concentrated under reduced pressure. The crude was purified by silica gel flash column chromatography using EtOAc in hexane (product eluted at 12% EtOAc in hexane) to obtain both isomers.

Peak 1. tert-Butyl(2s,4r)-2-(((benzyloxy)carbonyl)(isopropyl)amino)-5-azaspiro[3.5]nonane-5-carboxylate (102 mg, 34.5% yield) as a gummy liquid. ¹H NMR (400 MHz, CDCl₃): δ 7.40-7.30 (m, 5H), 5.15 (s, 2H), 4.18 (Sep, J=6.8 Hz, 1H), 4.00 (quin, J=9.1 Hz, 1H), 3.42-3.37 (m, 2H), 2.85-2.73 (m, 2H), 2.48 (br t, J=10.6 Hz, 2H), 1.66-1.58 (m, 2H), 1.53-1.50 (m, 2H), 1.48 (s, 9H), 1.40-1.32 (m, 2H), 1.17 (d, J=6.8 Hz, 6H); LCMS (Method B): Rt=2.86 min, m/z=317.2 [M+H-Boc]⁺, 99.90%.

Peak 2. tert-Butyl(2r,4s)-2-(((benzyloxy)carbonyl)(isopropyl)amino)-5-azaspiro[3.5]nonane-5-carboxylate (35 mg, 11.85% yield) as a gummy liquid. ¹H NMR (400 MHz, DMSO-d₆): δ 7.39-7.29 (m, 5H), 5.07 (s, 2H), 3.97-3.86 (m, 1H), 3.83-3.72 (m, 1H), 3.15-3.08 (m, 2H), 2.32-2.23 (m, 2H), 1.67-1.56 (m, 4H), 1.37 (s, 9H), 1.33-1.26 (m, 2H), 1.15 (d, J=6.9 Hz, 6H), two protons merged with solvent peaks; LCMS (Method B): Rt=2.79 min, m/z=417.3 [M+H]⁺, 99.89%.

### Step 2. tert-Butyl(2s,4r)-2-(isopropylamino)-5-azaspiro[3.5]nonane-5-carboxylate

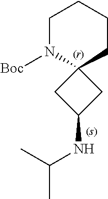 [SMILES: CC(C)N[C@H]1C[C@@]2(CCCCN2C(=O)OC(C)(C)C)C1]

In a 10 mL round bottom flask, tert-butyl(2s,4r)-2-(((benzyloxy)carbonyl)(isopropyl)amino)-5-azaspiro[3.5]nonane-5-carboxylate (90 mg, 0.216 mmol) was dissolved in TFE (4 mL), and Pd—C (90 mg, 0.085 mmol) was added at RT. The reaction was stirred at RT for 5 h under a hydrogen atmosphere (balloon pressure). The reaction progress was monitored by TLC (10% MeOH in DCM). After completion, the reaction was filtered through a Celite® pad, and the pad was washed with MeOH (2×20 mL). The filtrate was concentrated under reduced pressure, and reconcentrated from EtOAc (2×10 mL) then from toluene (2×10 mL), to obtain crude tert-butyl(2s,4r)-2-(isopropylamino)-5-azaspiro[3.5]nonane-5-carboxylate (46 mg, 74.8% yield) as a gummy liquid. This material was used without further purification. LCMS (Method B): Rt=1.36 min, m/z=283.2 [M+H]⁺, 99.23%.

### Step 3. tert-Butyl(2s,4r)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.5]nonane-5-carboxylate

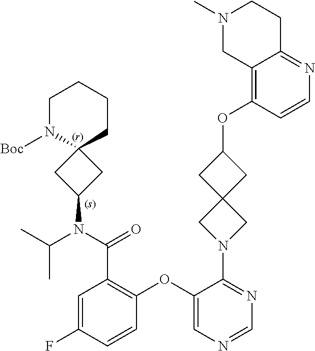 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCCN2C(=O)OC(C)(C)C)C1]

To a stirred solution of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid (75 mg, 0.153 mmol) and tert-butyl(2s,4r)-2-(isopropylamino)-5-azaspiro[3.5]nonane-5-carboxylate (43.1 mg, 0.153 mmol) in DMF (2 mL), DIPEA (0.080 mL, 0.458 mmol) and HATU (87 mg, 0.229 mmol) were added at 0° C. The reaction was stirred at RT for 2 h. The reaction was monitored by LCMS and TLC (10% MeOH in DCM). After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain tert-butyl(2s,4r)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.5]nonane-5-carboxylate (82 mg, 68.0% yield) as a solid. LCMS (Method H): Rt=1.44 min, m/z=756.4 [M+H]⁺, 95.63%.

### Step 4. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-azaspiro[3.5]nonan-2-yl)benzamide xHCl

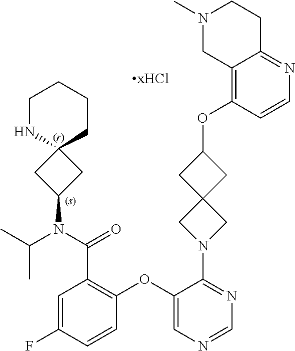

To a stirred solution of tert-butyl(2s,4r)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.5]nonane-5-carboxylate (82 mg, 0.108 mmol) in TFE (2 mL), TMSCl (0.055 mL, 0.434 mmol) was added at 0° C. The reaction was stirred at RT for 1 h. The reaction was monitored by LCMS. After completion, the reaction was concentrated under reduced pressure to afford crude 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-azaspiro[3.5]nonan-2-yl)benzamide xHCl (94 mg, quantitative yield) as a solid. This material was used without further purification. LCMS (Method H): Rt=0.97 min, m/z=656.4 [M+H]⁺, 98.65%.

### Step 5. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-methyl-5-azaspiro[3.5]nonan-2-yl)benzamide

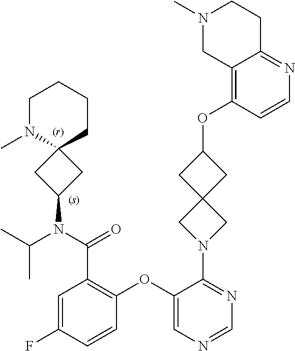 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCCN2C)C1]

To a stirred solution of 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-azaspiro[3.5]nonan-2-yl)benzamide xHCl (94 mg, 0.136 mmol) in DCM (5 mL), TEA (0.095 mL, 0.679 mmol) was added. The reaction was stirred at RT for 15 min, then was concentrated under reduced pressure. The residue was dissolved in MeOH (5 mL), and formaldehyde (0.019 mL, 0.679 mmol, 37% in H₂O) and acetic acid (0.777 μL, 0.014 mmol) were added at 0° C. The reaction was stirred at RT for 1 h, then sodium triacetoxyborohydride (57.6 mg, 0.272 mmol) was added at 0° C. The reaction was stirred at RT for 16 h. The reaction progress was monitored by LCMS. After completion, the reaction was concentrated under reduced pressure and the residue was quenched with aqueous NaHCO₃ solution (20 mL). The mixture was extracted with DCM (2×30 mL). The combined organic layer was dried over anhydrous sodium sulfate and filtered, and the filtrate was concentrated under reduced pressure to afford crude product. The crude was purified by reverse phase chromatography using a SiliCycle SiliaSep C18 column (120 g cartridge) on Biotage isolera one (Mobile phase A: 10 mM NH₄HCO₃ in water, B: ACN), then by reverse phase chromatography using a SiliCycle SiliaSep C18 column (120 g cartridge) on Biotage isolera one (Mobile phase A: 0.1% formic acid, B: ACN), to obtain corresponding formate salt. The compound was dissolved in 10% MeOH (20 mL) and the solution was basified using saturated sodium carbonate solution (10 mL). The organic layer was separated, dried over anhydrous sodium sulfate and filtered, and the filtrate was concentrated under reduced pressure and lyophilized to afford 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-methyl-5-azaspiro[3.5]nonan-2-yl)benzamide (25 mg, 27.1% yield) as a solid. ¹H NMR (400 MHz, DMSO-d₆): δ 8.30-8.23 (m, 1H), 8.20-8.16 (m, 1H), 7.80-7.53 (m, 1H), 7.32-7.05 (m, 3H), 6.70-6.61 (m, 1H), 4.75 (quin, J=6.8 Hz, 1H), 4.38-4.10 (m, 4H), 3.97-3.59 (m, 2H), 3.36 (br s, 2H), 2.86-2.70 (m, 4H), 2.63 (br t, J=5.9 Hz, 2H), 2.36 (s, 3H), 2.32-2.20 (m, 6H), 2.03-1.69 (m, 3H), 1.52-0.93 (m, 11H), three protons merged with solvent peaks; LCMS (Method H): Rt=0.99 min, m/z=670.4 [M+H]⁺; HPLC (Method G): Rt=6.40 min, 98.69%; Chiral SFC (Method C): Rt=4.85 min, 98.37%.

### Example 213. (D1)-5-Fluoro-N-isopropyl-N-(5-methyl-5-azaspiro[3.5]nonan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 213)

### Example 214. (D2)-5-Fluoro-N-isopropyl-N-(5-methyl-5-azaspiro[3.5]nonan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 214)

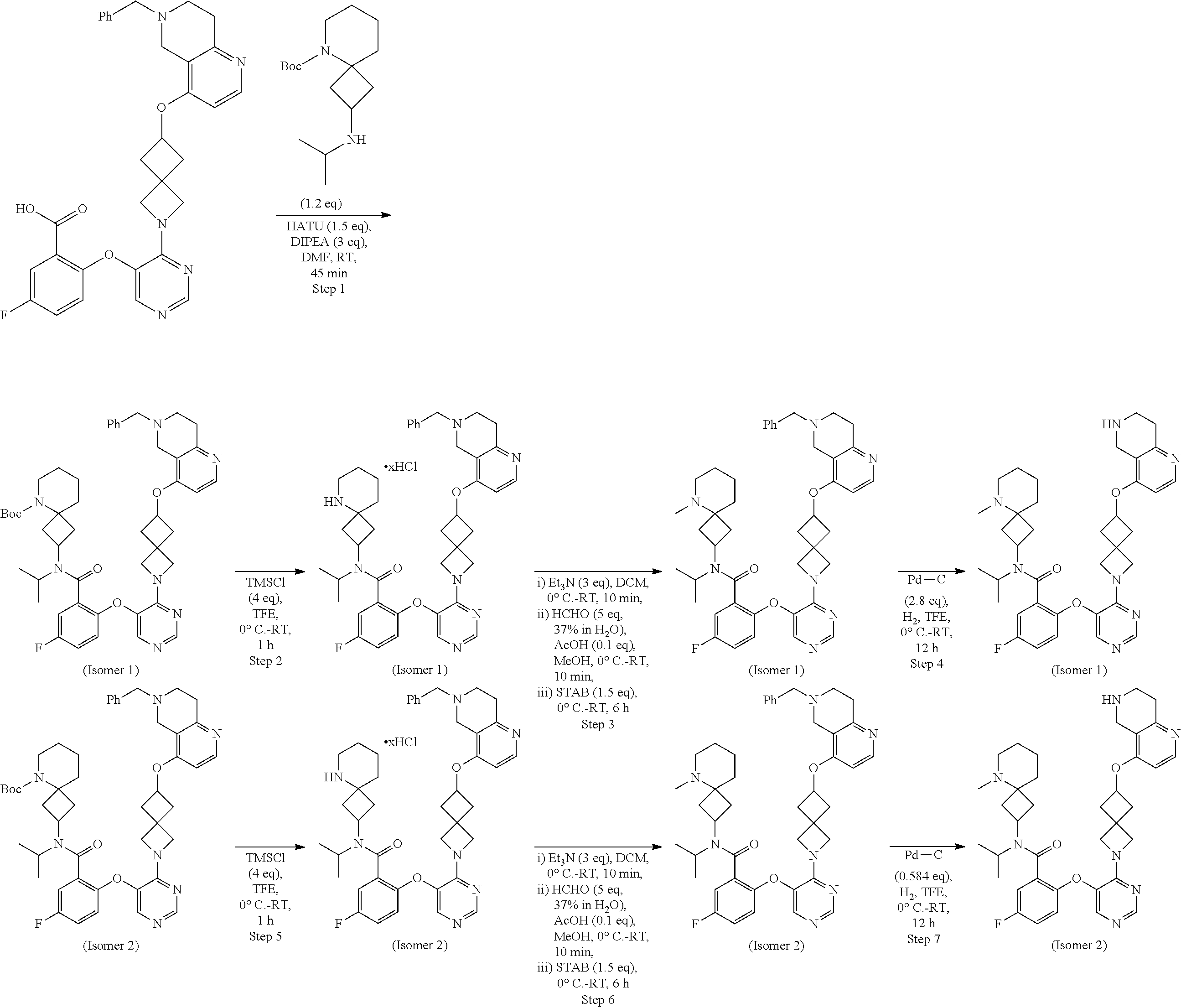

### Step 1. (D1)-tert-Butyl 2-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.5]nonane-5-carboxylate and (D2)-tert-butyl 2-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.5]nonane-5-carboxylate

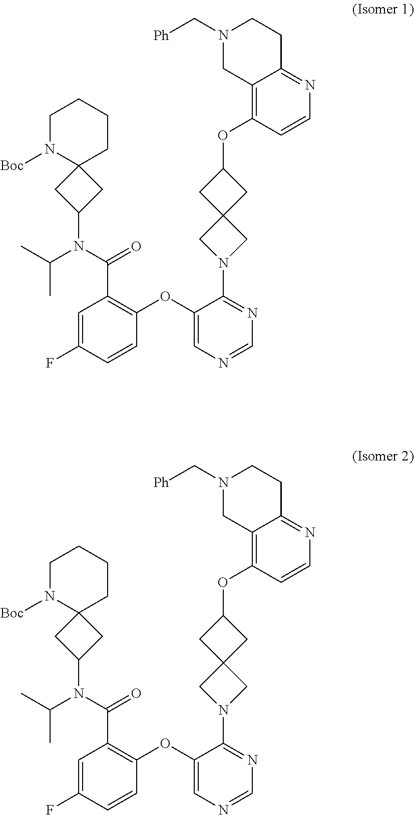 [SMILES: CC(C)N(C(=O)c1cc(F)ccc1Oc1cncnc1N1CC2(CC(Oc3ccnc4c3CN(Cc3ccccc3)CC4)C2)C1)C1CC2(CCCCN2C(=O)OC(C)(C)C)C1 ; CC(C)N(C(=O)c1cc(F)ccc1Oc1cncnc1N1CC2(CC(Oc3ccnc4c3CN(Cc3ccccc3)CC4)C2)C1)C1CC2(CCCCN2C(=O)OC(C)(C)C)C1]

This compound was synthesized following the procedure described for the synthesis of Examples 211 and 212, step 1, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.2 equivalent of tert-butyl 2-(isopropylamino)-5-azaspiro[3.5]nonane-5-carboxylate. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain a mixture of diastereomers, (D1)-tert-butyl 2-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.5]nonane-5-carboxylate and (D2)-tert-butyl 2-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.5]nonane-5-carboxylate.

Yield: 47.26%; LCMS (Method B): Rt=1.60 min; m/z=832.3 [M+H]⁺; HPLC (Method C): Peak 1; Rt=12.68 min, 44.25%; Peak 2; Rt=13.06 min, 54.66%.

The mixture of diastereomers was purified by Prep HPLC (Method A). The pure fractions were concentrated under reduced pressure to obtain both isomers.

Isomer 1. (D1)-tert-Butyl 2-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.5]nonane-5-carboxylate as a solid.

Yield: 13.72%; ¹H NMR (400 MHz, DMSO-d₆): δ 8.32-8.24 (m, 1H), 8.18 (br d, J=4.5 Hz, 1H), 7.79-7.62 (m, 1H), 7.41-7.01 (m, 8H), 6.66 (br d, J=5.4 Hz, 1H), 4.78-4.64 (m, 1H), 4.34-4.06 (m, 4H), 3.78-3.51 (m, 3H), 3.46 (br s, 2H), 3.26-3.01 (m, 3H), 2.86-2.64 (m, 6H), 2.28-1.90 (m, 5H), 1.71-0.97 (m, 21H), one proton merged with solvent peaks; LCMS (Method B): Rt=1.58 min; m/z=832.3 [M+H]⁺; HPLC (Method C): Rt=12.69 min, 99.05%.

Isomer 2. (D2)-tert-Butyl 2-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.5]nonane-5-carboxylate as a solid.

Yield: 17.85%: ¹H NMR (400 MHz, DMSO-d₆): δ 8.31-8.23 (m, 1H), 8.18 (br d, J=5.3 Hz, 1H), 7.80-7.64 (m, 11H), 7.38-7.03 (m, 8H), 6.69-6.60 (m, 1H), 4.72 (quin, J=6.4 Hz, 1H), 4.34-3.97 (m, 4H), 3.95-3.75 (m, 1H), 3.74-3.62 (m, 3H), 3.46 (br s, 2H), 3.30-3.18 (m, 2H), 3.01-2.89 (m, 1H), 2.84-2.60 (m, 8H), 2.20 (br s, 2H), 2.04-1.77 (m, 2H), 1.63-1.21 (m, 16H), 1.12-0.90 (m, 4H); LCMS (Method B): Rt=1.68-1.86 min; m/z=832.4 [M+H]⁺; HPLC (Method C): Rt=13.07 min, 97.86%.

### Step 2. (D1)-2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-azaspiro[3.5]nonan-2-yl)benzamide xHCl

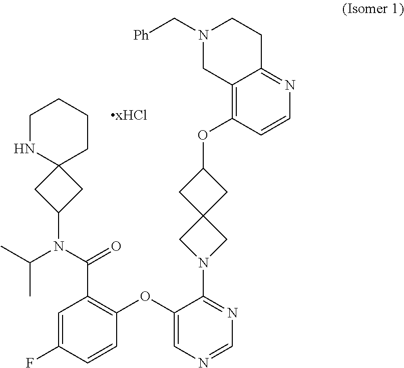 [SMILES: CC(C)N(C(=O)c1cc(F)ccc1Oc1cncnc1N1CC2(CC(Oc3ccnc4c3CN(Cc3ccccc3)CC4)C2)C1)C1CC2(CCCCN2)C1]

This compound was synthesized following the procedure described for the synthesis of Example 20, step 3, starting with 1 equivalent of (D1)-tert-butyl 2-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.5]nonane-5-carboxylate. After completion, the reaction was concentrated under reduced pressure, and the residue was triturated with EtOAc. The organic layer was decanted and the solid was dried under reduced pressure to obtain crude (D1)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-azaspiro[3.5]nonan-2-yl)benzamide xHCl as a solid. This material was used without further purification.

Yield: Quantitative; LCMS (Method B): Rt=1.20-1.33 min; m/z=732.4 [M+H]⁺; HPLC (Method C): Rt=9.94 min, 99.39%.

### Step 3. (D1)-2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-methyl-5-azaspiro[3.5]nonan-2-yl)benzamide

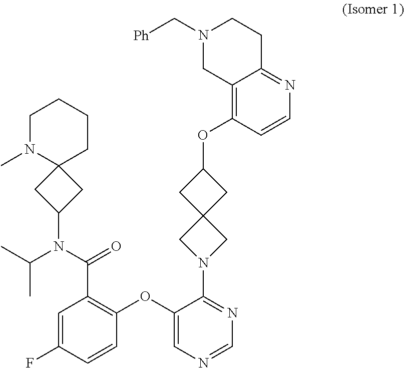 [SMILES: CC(C)N(C(=O)c1cc(F)ccc1Oc1cncnc1N1CC2(CC(Oc3ccnc4c3CN(Cc3ccccc3)CC4)C2)C1)C1CC2(CCCCN2C)C1]

This compound was synthesized following the procedure described for the synthesis of Example 20, step 4, starting with 1 equivalent of (D1)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-azaspiro[3.5]nonan-2-yl)benzamide xHCl, except that 5 equivalents of formaldehyde (37% in H₂O), 3 equivalents of TEA, 0.1 equivalent of AcOH, and 1.5 equivalents of STAB were used. After completion, the reaction was concentrated under reduced pressure. After work-up, the crude product was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to obtain (D1)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-methyl-5-azaspiro[3.5]nonan-2-yl)benzamide as a solid.

Yield: 58.4%; ¹H NMR (400 MHz, DMSO-d₆): δ 8.30-8.24 (m, 1H), 8.19 (d, J=5.6 Hz, 1H), 7.82-7.65 (m, 1H), 7.35 (d, J=4.4 Hz, 4H), 7.31-7.04 (m, 4H), 6.69-6.60 (m, 1H), 4.72 (quin, J=6.6 Hz, 1H), 4.34-4.08 (m, 4H), 3.75-3.54 (m, 4H), 3.47 (s, 2H), 3.13-3.03 (m, 1H), 2.86-2.66 (m, 6H), 2.31-2.13 (m, 6H), 1.86-1.55 (m, 3H), 1.51-1.20 (m, 8H), 1.13-0.94 (m, 3H), two protons merged with solvent peaks; LCMS (Method H): Rt=1.17 min; m/z=746.4 [M+H]⁺; HPLC (Method C): Rt=10.27 min, 99.41%.

### Step 4. (D1)-5-Fluoro-N-isopropyl-N-(5-methyl-5-azaspiro[3.5]nonan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

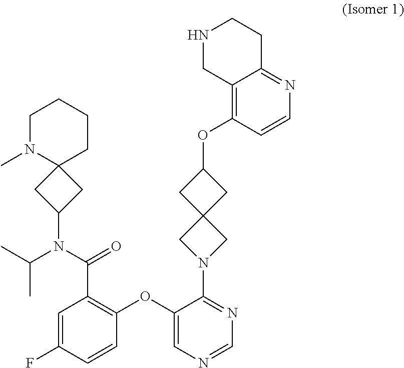 [SMILES: CC(C)N(C(=O)c1cc(F)ccc1Oc1cncnc1N1CC2(CC(Oc3ccnc4c3CNCC4)C2)C1)C1CC2(CCCCN2C)C1]

This compound was synthesized following the procedure described for the synthesis of Example 59, step 5, starting with 1 equivalent of (D1)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-methyl-5-azaspiro[3.5]nonan-2-yl)benzamide, except that 2.8 equivalents of Pd—C was used. After filtration, the crude product was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to obtain (D1)-5-fluoro-N-isopropyl-N-(5-methyl-5-azaspiro[3.5]nonan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 59%; ¹H NMR (400 MHz, DMSO-d₆): δ 8.33-8.22 (m, 1H), 8.16 (d, J=5.6 Hz, 1H), 7.82-7.64 (m, 1H), 7.32-7.02 (m, 3H), 6.69-6.57 (m, 1H), 4.72 (quin, J=6.4 Hz, 1H), 4.40-4.07 (m, 4H), 3.79-3.56 (m, 4H), 3.17-3.00 (m, 1H), 2.94 (br t, J=5.6 Hz, 2H), 2.87-2.73 (m, 2H), 2.67 (br t, J=5.1 Hz, 2H), 2.31-2.01 (m, 7H), 1.86-1.54 (m, 3H), 1.49-1.25 (m, 8H), 1.15-0.95 (m, 3H), one proton merged with solvent peaks; LCMS (Method H): Rt=1.04 min; m/z=656.3 [M+H]⁺; HPLC (Method G): Rt=6.34 min, 98.49%; Chiral SFC (Method AAP): Rt=1.90 min, 100%.

### Step 5. (D2)-2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-azaspiro[3.5]nonan-2-yl)benzamide xHCl

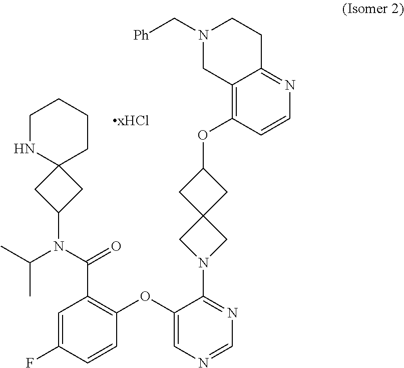 [SMILES: CC(C)N(C(=O)c1cc(F)ccc1Oc1cncnc1N1CC2(CC(Oc3ccnc4c3CN(Cc3ccccc3)CC4)C2)C1)C1CC2(CCCCN2)C1]

This compound was synthesized following the procedure described for the synthesis of Example 20, step 3, starting with 1 equivalent of (D2)-tert-butyl 2-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.5]nonane-5-carboxylate. After completion, the reaction was concentrated under reduced pressure, and the residue was triturated with EtOAc. The organic layer was decanted and the residue was dried under reduced pressure to obtain crude (D2)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-azaspiro[3.5]nonan-2-yl)benzamide hydrochloride as a solid. This material was used without further purification.

Yield: Quantitative; LCMS (Method B): Rt=1.04 min; m/z=732.4 [M+H]⁺; HPLC (Method H): Rt=6.18 min, 99.59%.

### Step 6. (D2)-2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-methyl-5-azaspiro[3.5]nonan-2-yl)benzamide

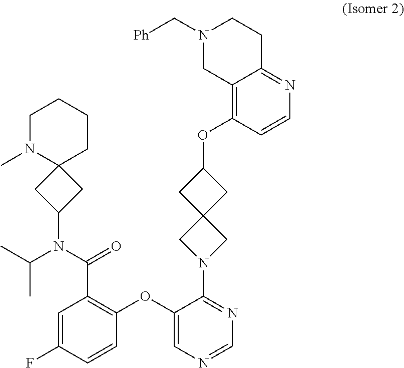 [SMILES: CC(C)N(C(=O)c1cc(F)ccc1Oc1cncnc1N1CC2(CC(Oc3ccnc4c3CN(Cc3ccccc3)CC4)C2)C1)C1CC2(CCCCN2C)C1]

This compound was synthesized following the procedure described for the synthesis of Example 20, step 4, starting with 1 equivalent of (D2)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-azaspiro[3.5]nonan-2-yl)benzamide xHCl, except that 5 equivalents of formaldehyde (37% in H₂O), 3 equivalents of TEA, 0.1 equivalent of AcOH, and 1.5 equivalents of STAB were used. After completion, the reaction was concentrated under reduced pressure. After work-up, the crude product was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to obtain (D2)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-methyl-5-azaspiro[3.5]nonan-2-yl)benzamide as a solid.

Yield: 51%; ¹H NMR (400 MHz, DMSO-d₆): δ 8.29-8.23 (m, 1H), 8.19 (br d, J=5.4 Hz, 1H), 7.80-7.56 (m, 1H), 7.38-7.06 (m, 8H), 6.71-6.57 (m, 1H), 4.72 (quin, J=6.4 Hz, 1H), 4.38-4.08 (m, 4H), 4.00-3.87 (m, 1H), 3.79-3.62 (m, 3H), 3.47 (s, 2H), 2.85-2.66 (m, 6H), 2.39-2.16 (m, 6H), 1.80-1.65 (m, 3H), 1.52-1.24 (m, 8H), 1.11-0.91 (m, 3H), three protons merged with solvent peaks; LCMS (Method H): Rt=1.16 min; m/z=746.4 [M+H]⁺; HPLC (Method C): Rt=10.47 min, 98.45%.

### Step 7. (D2)-5-Fluoro-N-isopropyl-N-(5-methyl-5-azaspiro[3.5]nonan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

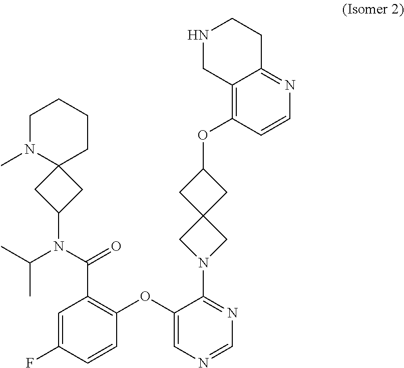 [SMILES: CC(C)N(C(=O)c1cc(F)ccc1Oc1cncnc1N1CC2(CC(Oc3ccnc4c3CNCC4)C2)C1)C1CC2(CCCCN2C)C1]

This compound was synthesized following the procedure described for the synthesis of Example 59, step 5, starting with 1 equivalent of (D2)-2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-methyl-5-azaspiro[3.5]nonan-2-yl)benzamide, except that 0.584 equivalent of Pd—C was used. After filtration, the solution was concentrated under reduced pressure. The crude product was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to obtain (D2)-5-fluoro-N-isopropyl-N-(5-methyl-5-azaspiro[3.5]nonan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide as a solid.

Yield: 56.3%; ¹H NMR (400 MHz, DMSO-d₆): δ 8.30-8.23 (m, 1H), 8.15 (br d, J=5.3 Hz, 1H), 7.80-7.56 (m, 1H), 7.32-7.05 (m, 3H), 6.67-6.57 (m, 1H), 4.72 (quin, J=6.3 Hz, 1H), 4.39-4.09 (m, 4H), 3.99-3.85 (m, 1H), 3.78-3.60 (m, 4H), 2.94 (br t, J=5.6 Hz, 2H), 2.85-2.72 (m, 2H), 2.67 (br t, J=5.0 Hz, 2H), 2.38-2.19 (m, 7H), 1.80-1.66 (m, 3H), 1.51-1.27 (m, 8H), 1.10-0.90 (m, 3H), one proton merged with solvent peaks; LCMS (Method H): Rt=1.02 min; m/z=656.3 [M+H]⁺; HPLC (Method G): Rt=6.23 min, 99.63%; Chiral SFC (Method AAP): Rt=1.98 min, 100%.

### Example 215. (D1)-2-((4-(6-((5-Ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-methyl-5-azaspiro[3.5]nonan-2-yl)benzamide (Compound No. 215)

### Example 216. (D2)-2-((4-(6-((5-Ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-methyl-5-azaspiro[3.5]nonan-2-yl)benzamide (Compound No. 216)

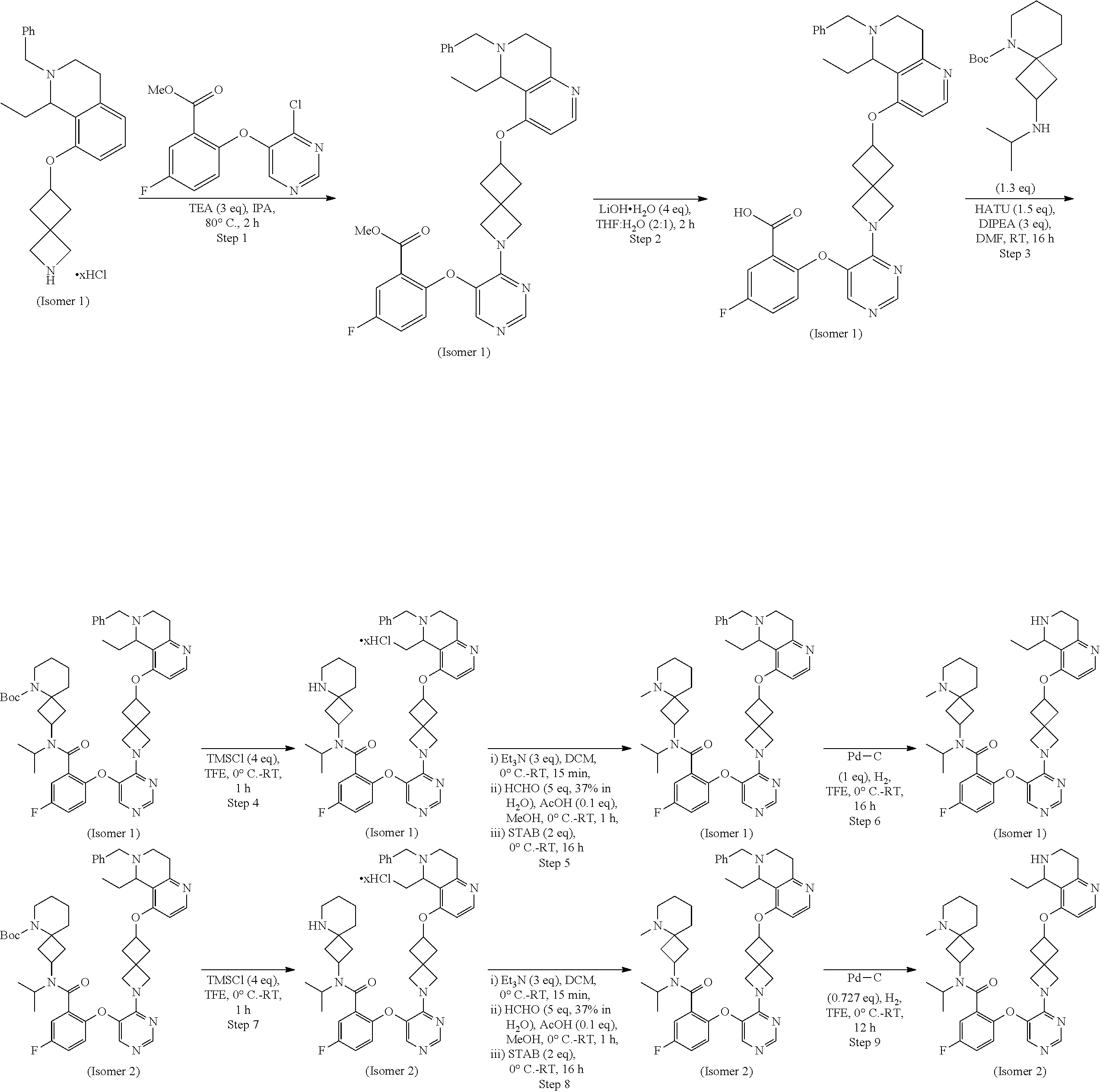

### Step 1. (E1)-Methyl 2-((4-(6-((6-benz yl-5-ethyl-5,6,7,8-tetrahydro-1,6-naphthyridin-4-yl)oxy)-2-azaspiro[3.3]heptan-2-yl)pyrimidin-5-yl)oxy)-5-fluorobenzoate

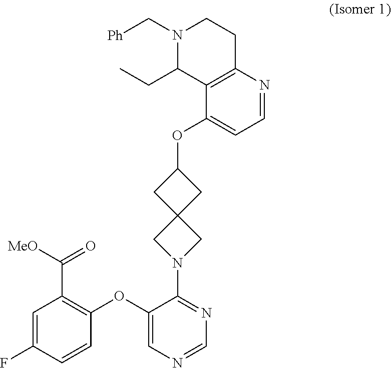 [SMILES: CCC1c2c(OC3CC4(C3)CN(c3ncncc3Oc3ccc(F)cc3C(=O)OC)C4)ccnc2CCN1Cc1ccccc1]

This compound was synthesized following the procedure described for the synthesis of Intermediate 22, step 1, starting with 1 equivalent of methyl 2-((4-chloropyrimidin-5-yl)oxy)-5-fluorobenzoate and 1 equivalent of (E1)-4-((2-azaspiro[3.3]heptan-6-yl)oxy)-6-benzyl-5-ethyl-5,6,7,8-tetrahydro-1,6-naphthyridine xHCl. After work-up, the crude product was purified by silica gel flash column chromatography using MeOH in DCM (product eluted at 7% MeOH in DCM) to obtain (E1)-methyl 2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluorobenzoate as a solid.

Yield: 73.0%; ¹H NMR (400 MHz, DMSO-d₆): δ 8.29 (s, 1H), 8.20 (d, J=5.6 Hz, 1H), 7.70-7.62 (m, 2H), 7.49 (ddd, J=3.3, 7.9, 9.0 Hz, 1H), 7.36-7.28 (m, 4H), 7.24 (dt, J=2.8, 5.7 Hz, 1H), 7.12 (dd, J=4.4, 9.1 Hz, 1H), 6.69 (d, J=5.6 Hz, 1H), 4.74 (quin, J=6.4 Hz, 1H), 4.31-4.23 (m, 2H), 4.22-4.14 (m, 2H), 3.80 (s, 3H), 3.69 (d, J=13.4 Hz, 1H), 3.60-3.56 (m, 1H), 3.53 (d, J=13.5 Hz, 1H), 3.22-3.11 (m, 1H), 2.96-2.85 (m, 1H), 2.83-2.72 (m, 3H), 2.48-2.45 (m, 1H), 2.24-2.12 (m, 2H), 1.64-1.48 (m, 2H), 0.91 (t, J=7.2 Hz, 3H); LCMS (Method A): Rt=2.30 min, m/z=610.7 [M+H]⁺, 95.15%.

### Step 2. (E1)-2-((4-(6-((6-Benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluorobenzoic Acid

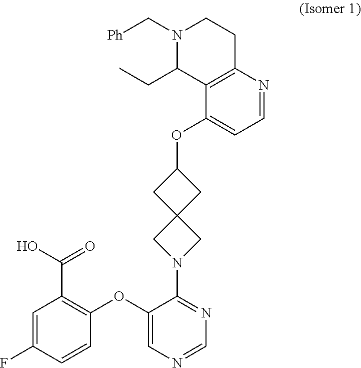 [SMILES: CCC1c2c(OC3CC4(C3)CN(c3ncncc3Oc3ccc(F)cc3C(=O)O)C4)ccnc2CCN1Cc1ccccc1]

This compound was synthesized following the procedure described for the synthesis of Intermediate 46, step 5, starting with 1 equivalent of (E1)-methyl 2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluorobenzoate, except that 4 equivalents of LiOH·H₂O was used, and THF:H₂O (2:1) was used as the solvent. After work-up, the mixture was extracted with 10% MeOH in DCM. The combined organic layer was dried over sodium sulfate and filtered, and the filtrate was concentrated under reduced pressure to obtain crude (E1)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluorobenzoic acid as a solid. This material was used without further purification.

Yield: 80%; LCMS (Method A): Rt=1.50 min, m/z=596.2 [M+H]⁺, 96.19%.

### Step 3. (D1)-tert-Butyl 2-(2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.5]nonane-5-carboxylate and (D2)-tert-butyl 2-(2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.5]nonane-5-carboxylate

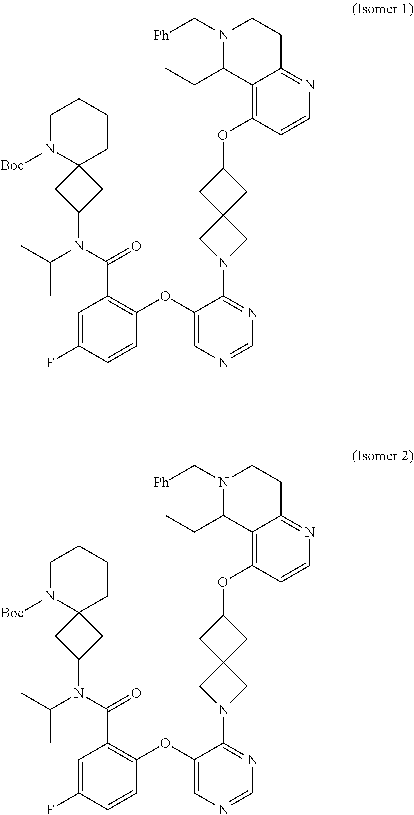 [SMILES: CCC1c2c(OC3CC4(C3)CN(c3ncncc3Oc3ccc(F)cc3C(=O)N(C(C)C)C3CC5(CCCCN5C(=O)OC(C)(C)C)C3)C4)ccnc2CCN1Cc1ccccc1 ; CCC1c2c(OC3CC4(C3)CN(c3ncncc3Oc3ccc(F)cc3C(=O)N(C(C)C)C3CC5(CCCCN5C(=O)OC(C)(C)C)C3)C4)ccnc2CCN1Cc1ccccc1]

This compound was synthesized following the procedure described for the synthesis of Examples 211 and 212, step 1, starting with 1 equivalent of (E1)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.3 equivalents of tert-butyl 2-(isopropylamino)-5-azaspiro[3.5]nonane-5-carboxylate. After completion, the reaction was concentrated under reduced pressure. The crude product was purified by reverse phase chromatography using a SiliCycle SiliaSep C18 column (80 g) on Biotage isolera one (Mobile phase A: 10 mM NH₄HCO₃ in water, B: ACN) to obtain a mixture of diastereomers, (D1)-tert-butyl 2-(2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.5]nonane-5-carboxylate and (D2)-tert-butyl 2-(2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.5]nonane-5-carboxylate, as a solid.

Yield: 70.6%; LCMS (Method A): Peak 1; Rt=2.70 min, 54.71%; Peak 2; Rt=2.77 min, 45.29%; m/z=860.4 [M+H]⁺; HPLC (Method C): Peak 1; Rt=13.66 min, 50.61%; Peak 2;Rt=14.01 min, 42.02%.

The mixture of diastereomers was purified by Prep HPLC (Method O). The pure fractions were concentrated under reduced pressure to obtain both isomers.

Isomer 1. (D1)-tert-Butyl 2-(2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.5]nonane-5-carboxylate as a solid.

Yield: 24.39%; LCMS (Method A): Rt=2.70 min; m/z=860.4 [M+H]⁺; HPLC (Method C): Rt=13.65 min, 99.49%.

Isomer 2. (D2) tert-Butyl 2-(2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.5]nonane-5-carboxylate as a solid.

Yield: 23.70%; LCMS (Method A): Rt=2.78 min; m/z=860.4 [M+H]⁺; HPLC (Method C): Rt=14.01 min, 97.79%.

### Step 4. (D1)-2-((4-(6-((6-Benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-azaspiro[3.5]nonan-2-yl)benzamide xHCl

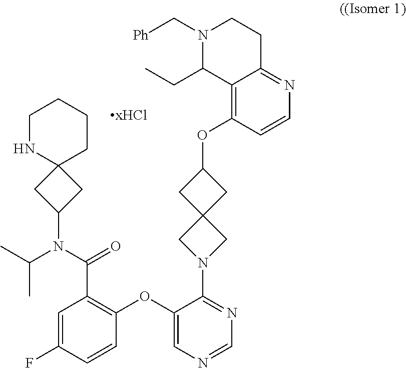 [SMILES: CCC1c2c(OC3CC4(C3)CN(c3ncncc3Oc3ccc(F)cc3C(=O)N(C(C)C)C3CC5(CCCCN5)C3)C4)ccnc2CCN1Cc1ccccc1]

This compound was synthesized following the procedure described for the synthesis of Example 20, step 3, starting with 1 equivalent of (D1)-2-(2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.5]nonane-5-carboxylate. After completion, the reaction was concentrated under reduced pressure, and the residue was triturated with EtOAc. The organic layer was decanted and the solid was dried under reduced pressure to obtain crude (D1)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-azaspiro[3.5]nonan-2-yl)benzamide xHCl as a solid. This material was used without further purification.

Yield: Quantitative; LCMS (Method H): Rt=1.22 min, m/z=760.3 [M+H]⁺, 96.14%.

### Step 5. (D1)-2-((4-(6-((6-Benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-methyl-5-azaspiro[3.5]nonan-2-yl)benzamide

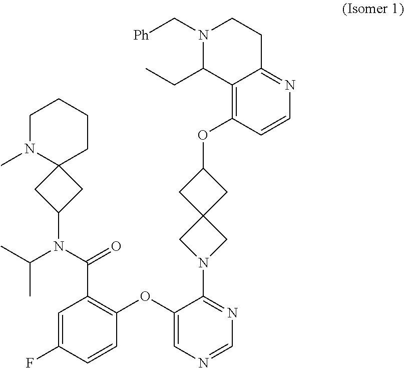 [SMILES: CCC1c2c(OC3CC4(C3)CN(c3ncncc3Oc3ccc(F)cc3C(=O)N(C(C)C)C3CC5(CCCCN5C)C3)C4)ccnc2CCN1Cc1ccccc1]

This compound was synthesized following the procedure described for the synthesis of Example 20, step 4, starting with 1 equivalent of (D1)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-azaspiro[3.5]nonan-2-yl)benzamide xHCl, except that 5 equivalents of formaldehyde (37% in H₂O), 3 equivalents of TEA, 0.1 equivalent of AcOH, and 2 equivalents of STAB were used. After completion, the reaction was concentrated under reduced pressure. After work-up, the crude product was purified by reverse phase chromatography using a SiliCycle SiliaSep C18 column (40 g) on Biotage isolera one (Mobile phase A: 10 mM NH₄HCO₃ in water, B: ACN) to afford (D1)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-methyl-5-azaspiro[3.5]nonan-2-yl)benzamide as a solid.

Yield: 49.3%; LCMS (Method H): Rt=1.23 min, m/z=774.4 [M+H]⁺, 99.89%.

### Step 6. (D1)-2-((4-(6-((5-Ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-methyl-5-azaspiro[3.5]nonan-2-yl)benzamide

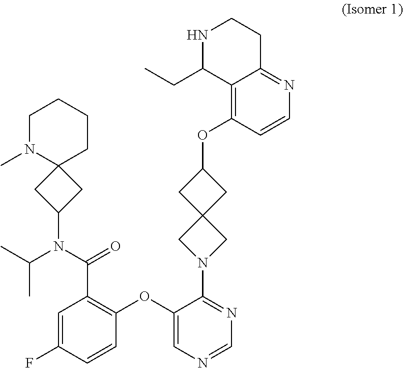 [SMILES: CCC1NCCc2nccc(OC3CC4(C3)CN(c3ncncc3Oc3ccc(F)cc3C(=O)N(C(C)C)C3CC5(CCCCN5C)C3)C4)c21]

This compound was synthesized following the procedure described for the synthesis of Example 59, step 5, starting with 1 equivalent of (D1)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-methyl-5-azaspiro[3.5]nonan-2-yl)benzamide, except that 1 equivalent of Pd—C was used. After completion, the reaction was filtered through a Celite® pad, and the pad was washed with MeOH. The filtrate was concentrated under reduced pressure. The crude product was purified by reverse phase chromatography using a SiliCycle SiliaSep C18 column (12 g) on Biotage isolera one (Mobile phase A: 10 mM NH₄HCO₃ in water, B: ACN). The pure fractions were concentrated under reduced pressure and lyophilized to obtain (D1)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-methyl-5-azaspiro[3.5]nonan-2-yl)benzamide as a solid.

Yield: 47.8%; ¹H NMR (400 MHz, DMSO-d₆): δ 8.32-8.22 (m, 1H), 8.15 (d, J=5.5 Hz, 1H), 7.82-7.63 (m, 1H), 7.31-7.02 (m, 3H), 6.68-6.58 (m, 1H), 4.78-4.65 (m, 1H), 4.41-4.06 (m, 4H), 3.81-3.54 (m, 3H), 3.13-2.73 (m, 5H), 2.66-2.60 (m, 1H), 2.40-2.35 (m, 1H), 2.30-1.98 (m, 7H), 1.82-1.21 (m, 14H), 1.13-0.86 (m, 7H); LCMS (Method B): Rt=1.07 min; m/z=684.4 [M+H]⁺; HPLC (Method G): Rt=6.56 min, 99.21%; Chiral SFC (Method AZ): Rt=6.41 min, 100%.

### Step 7. (D2)-2-((4-(6-((6-Benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-azaspiro[3.5]nonan-2-yl)benzamide xHCl

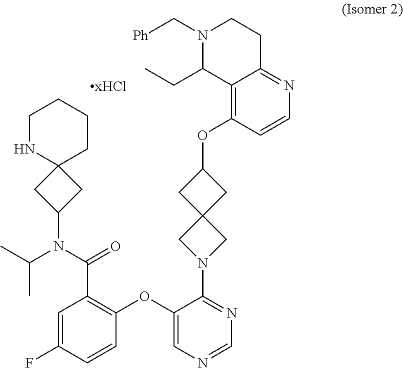 [SMILES: CCC1c2c(OC3CC4(C3)CN(c3ncncc3Oc3ccc(F)cc3C(=O)N(C(C)C)C3CC5(CCCCN5)C3)C4)ccnc2CCN1Cc1ccccc1]

This compound was synthesized following the procedure described for the synthesis of Example 20, step 3, starting with 1 equivalent of (D2)-2-(2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-5-azaspiro[3.5]nonane-5-carboxylate. After completion, the reaction was concentrated under reduced pressure, and the residue was triturated with EtOAc. The organic layer was decanted and the solid was dried under reduced pressure to obtain crude (D2)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-azaspiro[3.5]nonan-2-yl)benzamide xHCl as a solid. This material was used without further purification.

Yield: Quantitative; LCMS (Method H): Rt=1.19 min, m/z=760.3 [M+H]⁺, 99.04%.

### Step 8. (D2)-2-((4-(6-((6-Benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-methyl-5-azaspiro[3.5]nonan-2-yl)benzamide

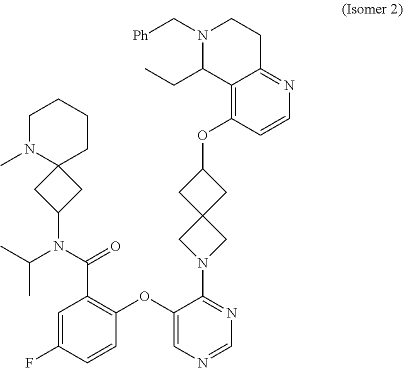 [SMILES: CCC1c2c(OC3CC4(C3)CN(c3ncncc3Oc3ccc(F)cc3C(=O)N(C(C)C)C3CC5(CCCCN5C)C3)C4)ccnc2CCN1Cc1ccccc1]

This compound was synthesized following the procedure described for the synthesis of Example 20, step 4, starting with 1 equivalent of (D2)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-azaspiro[3.5]nonan-2-yl)benzamide xHCl, except that 5 equivalents of formaldehyde (37% in H₂O), 3 equivalents of TEA, 0.1 equivalent of AcOH, and 2 equivalents of STAB were used. After completion, the reaction was concentrated under reduced pressure. After work-up, the crude product was purified by reverse phase chromatography using a SiliCycle SiliaSep C18 column (40 g) on Biotage isolera one (Mobile phase A: 10 mM NH₄HCO₃ in water, B: ACN) to afford (D2)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-methyl-5-azaspiro[3.5]nonan-2-yl)benzamide as a solid.

Yield: 49.3%; LCMS (Method H): Rt=1.22 min, m/z=774.4 [M+H]⁺, 99.80%.

### Step 6. (D2)-2-((4-(6-((5-Ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-methyl-5-azaspiro[3.5]nonan-2-yl)benzamide

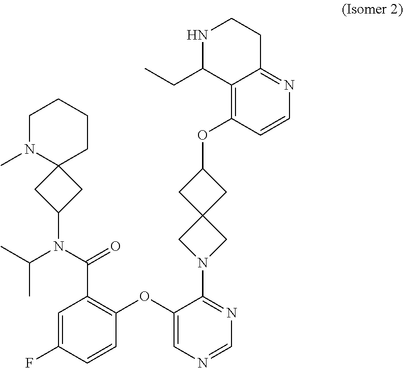 [SMILES: CCC1NCCc2nccc(OC3CC4(C3)CN(c3ncncc3Oc3ccc(F)cc3C(=O)N(C(C)C)C3CC5(CCCCN5C)C3)C4)c21]

This compound was synthesized following the procedure described for the synthesis of Example 59, step 5, starting with 1 equivalent of (D2)-2-((4-(6-((6-benzyl-5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-methyl-5-azaspiro[3.5]nonan-2-yl)benzamide, except that 0.727 equivalent of Pd—C was used. After completion, the reaction was filtered through a Celite® pad, the pad was washed with MeOH, and the filtrate was concentrated under reduced pressure. The crude product was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to obtain (D2)-2-((4-(6-((5-ethyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(5-methyl-5-azaspiro[3.5]nonan-2-yl)benzamide as a solid.

Yield: 51.6%; ¹H NMR (400 MHz, DMSO-d₆): δ 8.29-8.24 (m, 1H), 8.17-8.12 (m, 1H), 7.80-7.55 (m, 1H), 7.33-7.06 (m, 3H), 6.75-6.56 (m, 1H), 4.79-4.66 (m, 1H), 4.40-4.09 (m, 4H), 3.96-3.60 (m, 3H), 3.04-2.72 (m, 5H), 2.66-2.58 (m, 2H), 2.31-2.16 (m, 7H), 2.05-1.95 (m, 2H), 1.79-1.22 (m, 12H), 1.09-0.89 (m, 7H); LCMS (Method H): Rt=1.05 min; m/z=684.4 [M+H]⁺; HPLC (Method G): Rt=6.47 min, 99.69%, Chiral SFC (Method AZ): Rt=6.73 min, 99.22%.

### Example 217. (D1)-5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(5-methyl-8-oxa-5-azaspiro[3.5]nonan-2-yl)benzamide (Compound No. 217)

### Example 218. (D2)-5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(5-methyl-8-oxa-5-azaspiro[3.5]nonan-2-yl)benzamide (Compound No. 218)

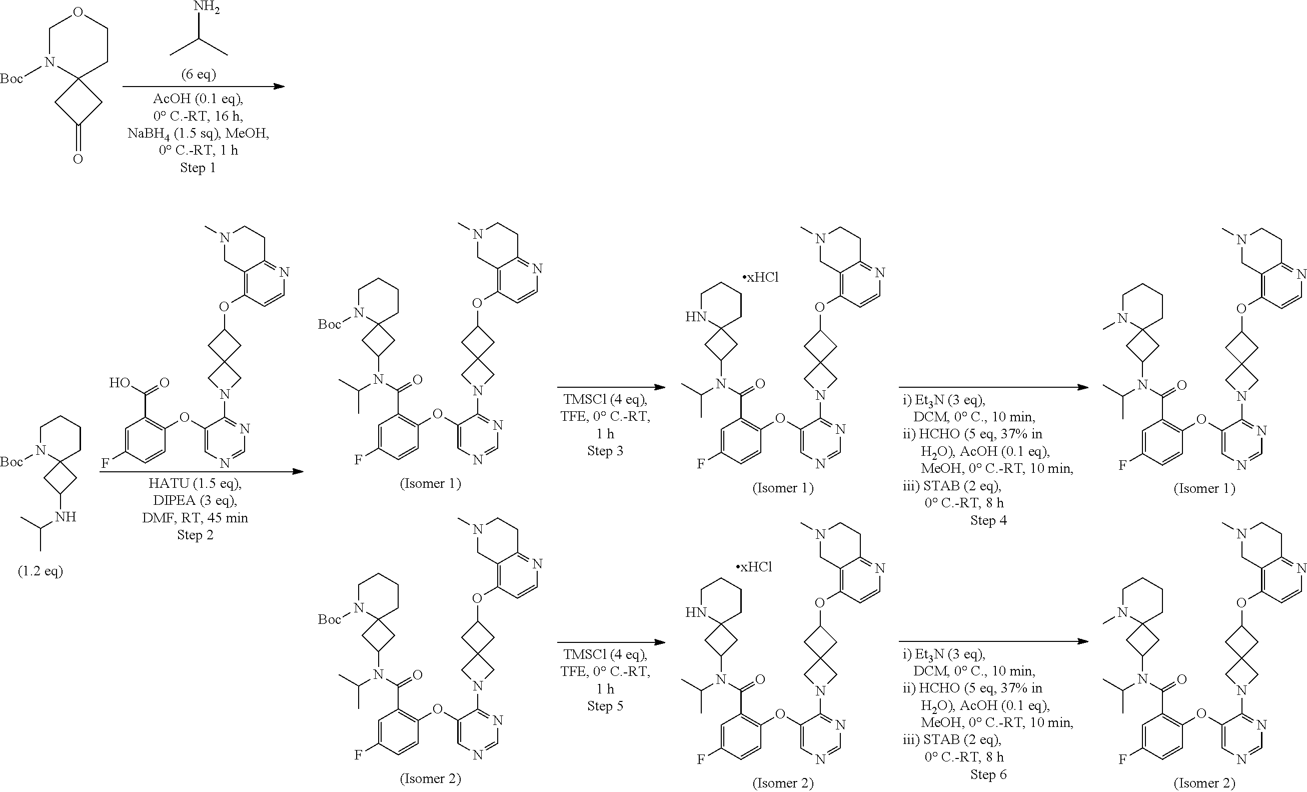

### Step 1. tert-Butyl 2-(isopropylamino)-8-oxa-5-azaspiro[3.5]nonane-5-carboxylate

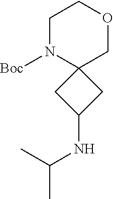 [SMILES: CC(C)NC1CC2(COCCN2C(=O)OC(C)(C)C)C1]

This compound was synthesized following the procedure described for the synthesis of Intermediate 51, starting with 1 equivalent of tert-butyl 2-oxo-8-oxa-5-azaspiro[3.5]nonane-5-carboxylate. After completion, the reaction was concentrated under reduced pressure. The residue was diluted with water and the mixture was extracted with EtOAc. The combined organic layer was dried over sodium sulfate and filtered, and the filtrate was concentrated under reduced pressure to obtain crude tert-butyl 2-(isopropylamino)-8-oxa-5-azaspiro[3.5]nonane-5-carboxylate as a gummy liquid. This material was used without further purification

Yield: 87%; LCMS (Method H): Peak 1; Rt=1.21 min, 66.61%; Peak 2; Rt=1.30 min, 32.71%; m/z=285.1 [M+H]⁺; HPLC (Method C): Peak 1; Rt=7.75 min, 62.82%; Peak 2; Rt=8.20 min, 34.30%.

### Step 2. (D1)-tert-Butyl 2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-8-oxa-5-azaspiro[3.5]nonane-5-carboxylate and (D2)-tert-butyl 2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-8-oxa-5-azaspiro[3.5]nonane-5-carboxylate

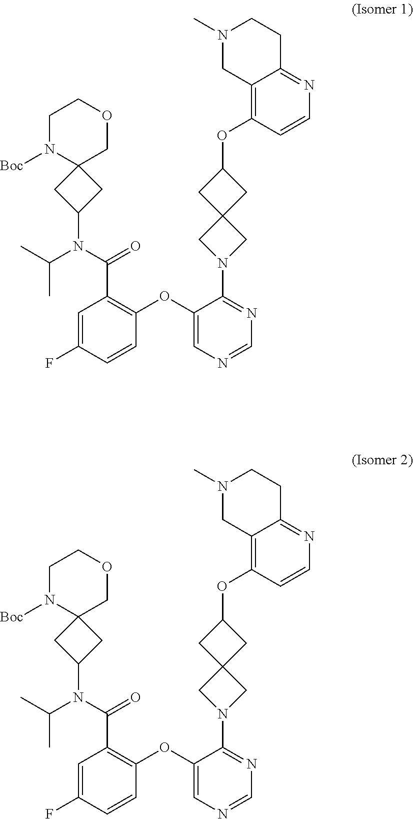 [SMILES: CC(C)N(C(=O)c1cc(F)ccc1Oc1cncnc1N1CC2(CC(Oc3ccnc4c3CN(C)CC4)C2)C1)C1CC2(COCCN2C(=O)OC(C)(C)C)C1 ; CC(C)N(C(=O)c1cc(F)ccc1Oc1cncnc1N1CC2(CC(Oc3ccnc4c3CN(C)CC4)C2)C1)C1CC2(COCCN2C(=O)OC(C)(C)C)C1]

This compound was synthesized following the procedure described for the synthesis of Examples 211 and 212, step 1, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.2 equivalents of tert-butyl 2-(isopropylamino)-8-oxa-5-azaspiro[3.5]nonane-5-carboxylate. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain a mixture of diastereomers, (D1)-tert-butyl 2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-8-oxa-5-azaspiro[3.5]nonane-5-carboxylate and (D2)-tert-butyl 2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-8-oxa-5-azaspiro[3.5]nonane-5-carboxylate.

Yield: 61.88%; ¹H NMR (400 MHz, DMSO-d₆): δ 8.32-8.23 (m, 1H), 8.18 (d, J=5.6 Hz, 1H), 7.82-7.71 (m, 1H), 7.35-7.01 (m, 3H), 6.66 (d, J=5.6 Hz, 1H), 4.74 (quin, J=6.7 Hz, 1H), 4.35-4.06 (m, 4H), 3.84-3.54 (m, 4H), 3.49-3.39 (m, 2H), 3.30-3.24 (m, 2H), 2.86-2.76 (m, 4H), 2.70-2.59 (m, 2H), 2.36 (s, 3H), 2.30-2.19 (m, 3H), 1.80-1.72 (m, 1H), 1.47-1.26 (m, 13H), 1.12-0.91 (m, 6H); LCMS (Method A): Peak 1; Rt=2.01 min, 81.30%; Peak 2;Rt=2.14 min, 17.06%; m/z=758.3 [M+H]⁺; HPLC (Method C): Peak 1; Rt=9.80 min, 83.75%; Peak 2; Rt=10.35 min, 14.97%; Chiral SFC (Method AAR): Peak 1; Rt=7.42 min, 84.04%; Peak 2; Rt=8.95 min, 15.96%.

The mixture of diastereomers was purified by chiral Prep SFC (Method AB). The pure fractions were concentrated under reduced pressure to obtain pure isomer 1 and impure isomer 2.

Isomer 1. (D1)-tert-Butyl 2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-8-oxa-5-azaspiro[3.5]nonane-5-carboxylate as a solid.

Yield: 39.7%; ¹H NMR (400 MHz, DMSO-d₆): δ 8.33-8.23 (m, 1H), 8.18 (d, J=5.5 Hz, 1H), 7.81-7.72 (m, 1H), 7.34-7.01 (m, 3H), 6.68-6.63 (m, 1H), 4.74 (quin, J=6.7 Hz, 1H), 4.35-4.07 (m, 4H), 3.85-3.54 (m, 4H), 3.46-3.42 (m, 2H), 3.28-3.18 (m, 2H), 2.84-2.74 (m, 4H), 2.63-2.60 (m, 2H), 2.36 (s, 3H), 2.32-2.22 (m, 3H), 1.82-1.74 (m, 1H), 1.47-1.28 (m, 13H), 1.13-0.92 (m, 6H); LCMS (Method H): Rt=1.30 min, m/z=758.3 [M+H]⁺; HPLC (Method C): Rt=9.78 min, 98.36%.

Isomer 2. This compound was re-purified by Prep HPLC (Method A) to obtain (D2)-tert-butyl 2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-8-oxa-5-azaspiro[3.5]nonane-5-carboxylate as solid.

Yield: 1.96%; ¹H NMR (400 MHz, DMSO-d₆): δ 8.28 (s, 1H), 8.18 (d, J=5.6 Hz, 1H), 7.82-7.73 (m, 1H), 7.35-7.04 (m, 3H), 6.65 (br d, J=5.5 Hz, 1H), 4.80-4.67 (m, 1H), 4.36-4.08 (m, 5H), 3.88-3.68 (m, 2H), 3.55-3.42 (m, 3H), 3.36 (br s, 2H), 3.28-3.20 (m, 1H), 3.02-2.92 (m, 1H), 2.85-2.73 (m, 5H), 2.63 (br t, J=5.5 Hz, 2H), 2.36 (s, 3H), 2.29-2.02 (m, 3H), 1.50-1.23 (m, 11H), 1.12-0.91 (m, 4H), two protons merged with solvent peaks; LCMS (Method H): Rt=1.40 min, m/z=758.4 [M+H]⁺, 98.64%.

### Step 3. (D1)-5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(8-oxa-5-azaspiro[3.5]nonan-2-yl)benzamide xHCl

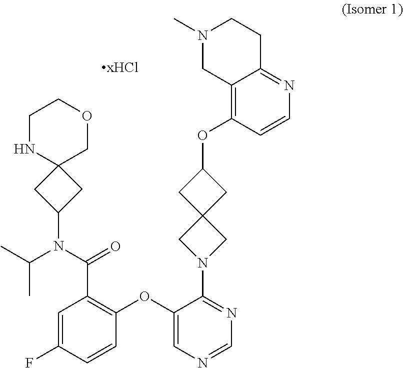 [SMILES: CC(C)N(C(=O)c1cc(F)ccc1Oc1cncnc1N1CC2(CC(Oc3ccnc4c3CN(C)CC4)C2)C1)C1CC2(COCCN2)C1]

This compound was synthesized following the procedure described for the synthesis of Example 20, step 3, starting with 1 equivalent of (D1)-tert-butyl 2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-8-oxa-5-azaspiro[3.5]nonane-5-carboxylate. After completion, the reaction was concentrated under reduced pressure, and the residue was triturated with EtOAc. The organic layer was decanted and the solid was dried under reduced pressure to obtain crude (D1)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(8-oxa-5-azaspiro[3.5]nonan-2-yl)benzamide xHCl as a solid. This material was used without further purification.

Yield: Quantitative; LCMS (Method A): Rt=1.72 min, m/z=658.2 [M+H]⁺; HPLC (Method C): Rt=7.98 min, 97.90%.

### Step 4. (D1)-5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(5-methyl-8-oxa-5-azaspiro[3.5]nonan-2-yl)benzamide

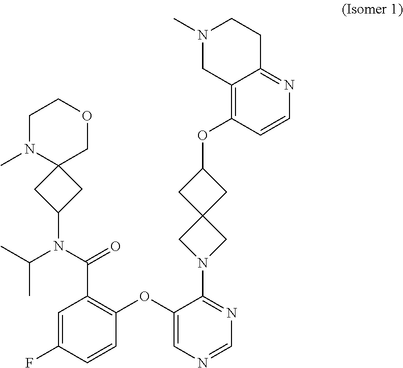 [SMILES: CC(C)N(C(=O)c1cc(F)ccc1Oc1cncnc1N1CC2(CC(Oc3ccnc4c3CN(C)CC4)C2)C1)C1CC2(COCCN2C)C1]

This compound was synthesized following the procedure described for the synthesis of Example 20, step 4, starting with 1 equivalent of (D1)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(8-oxa-5-azaspiro[3.5]nonan-2-yl)benzamide xHCl, except that 5 equivalents of formaldehyde (37% in H₂O), 3 equivalents of TEA, 0.1 equivalent of AcOH, and 2 equivalents of STAB were used. After completion, the reaction was concentrated under reduced pressure. After work-up, the crude product was purified by reverse phase chromatography using a SiliCycle SiliaSep C18 column (120 g) on Biotage isolera one (Mobile phase A: 10 mM NH₄HCO₃ in water, B: ACN). The pure fractions were concentrated under reduced pressure and lyophilized to obtain (D1)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(5-methyl-8-oxa-5-azaspiro[3.5]nonan-2-yl)benzamide as a solid.

Yield: 37.3%; ¹H NMR (400 MHz, DMSO-d₆): δ 8.32-8.25 (m, 1H), 8.21-8.16 (m, 1H), 7.81-7.67 (m, 1H), 7.31-7.02 (m, 3H), 6.64 (br d, J=5.5 Hz, 1H), 4.74 (quin, J=6.6 Hz, 1H), 4.36-4.08 (m, 4H), 3.83-3.61 (m, 2H), 3.60-3.45 (m, 4H), 3.36 (br s, 2H), 3.28-3.09 (m, 1H), 2.84-2.74 (m, 4H), 2.65-2.60 (m, 2H), 2.36 (s, 3H), 2.32 (s, 3H), 2.30-2.21 (m, 5H), 1.83-1.57 (m, 2H), 1.49-0.95 (m, 6H); LCMS (Method H): Rt=0.99 min, m/z=672.3 [M+H]⁺; HPLC (Method C): Rt=8.20 min, 99.75%.

### Step 5. (D2)-5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(8-oxa-5-azaspiro[3.5]nonan-2-yl)benzamide xHCl

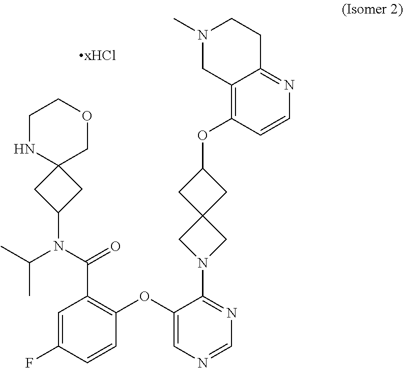 [SMILES: CC(C)N(C(=O)c1cc(F)ccc1Oc1cncnc1N1CC2(CC(Oc3ccnc4c3CN(C)CC4)C2)C1)C1CC2(COCCN2)C1]

This compound was synthesized following the procedure described for the synthesis of Example 20, step 3, starting with 1 equivalent of (D2)-tert-butyl 2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-8-oxa-5-azaspiro[3.5]nonane-5-carboxylate. After completion, the reaction was concentrated under reduced pressure, and the residue was triturated with EtOAc. The organic layer was decanted and the solid was dried under reduced pressure to obtain crude (D2)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(8-oxa-5-azaspiro[3.5]nonan-2-yl)benzamide xHCl as a solid.

Yield: Quantitative; LCMS (Method B): Rt=0.77 min, m/z=658.3 [M+H]⁺, 98.43%.

### Step 6. (D2)-5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(5-methyl-8-oxa-5-azaspiro[3.5]nonan-2-yl)benzamide

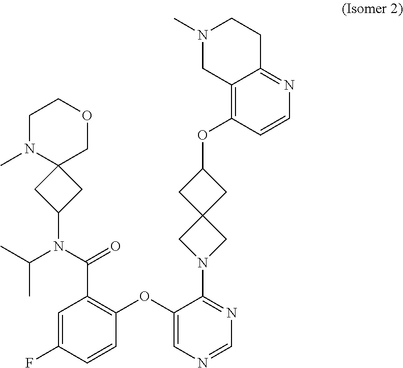 [SMILES: CC(C)N(C(=O)c1cc(F)ccc1Oc1cncnc1N1CC2(CC(Oc3ccnc4c3CN(C)CC4)C2)C1)C1CC2(COCCN2C)C1]

This compound was synthesized following the procedure described for the synthesis of Example 20, step 4, starting with 1 equivalent of (D2)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(8-oxa-5-azaspiro[3.5]nonan-2-yl)benzamide xHCl, except that 5 equivalents of formaldehyde (37% in H₂O), 3 equivalents of TEA, 0.1 equivalent of AcOH, and 2 equivalents of STAB were used. After completion, the reaction was concentrated under reduced pressure. After work-up, the crude product was purified by reverse phase chromatography using a SiliCycle SiliaSep C18 column (120 g) on Biotage isolera one (Mobile phase A: 10 mM NH₄HCO₃ in water, B: ACN). The pure fractions were concentrated under reduced pressure and lyophilized to obtain (D2)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(5-methyl-8-oxa-5-azaspiro[3.5]nonan-2-yl)benzamide as a solid.

Yield: 34.8%; ¹H NMR (400 MHz, DMSO-d₆): δ 8.32-8.23 (m, 1H), 8.21-8.14 (m, 1H), 7.81-7.54 (m, 1H), 7.35-7.06 (m, 3H), 6.65 (br d, J=5.5 Hz, 1H), 4.74 (quin, J=6.4 Hz, 1H), 4.38-4.10 (m, 4H), 4.01-3.57 (m, 4H), 3.56-3.42 (m, 2H), 3.36 (br s, 2H), 2.85-2.75 (m, 4H), 2.63 (br t, =5.7 Hz, 2H), 2.36 (s, 3H), 2.33 (s, 3H), 2.26 (br s, 5H), 2.02-1.70 (m, 2H), 1.49-0.89 (m, 6H), one proton merged with solvent peaks; LCMS (Method H): Rt=1.01 min, m/z=672.3 [M+H]⁺; HPLC (Method G): Rt=6.24 min, 99.31%.

### Example 219. (D1)-5-Fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-(5-methyl-5-azaspiro[3.5]nonan-2-yl)benzamide (Compound No. 219)

### Example 220. (D2)-5-Fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-(5-methyl-5-azaspiro[3.5]nonan-2-yl)benzamide (Compound No. 220)

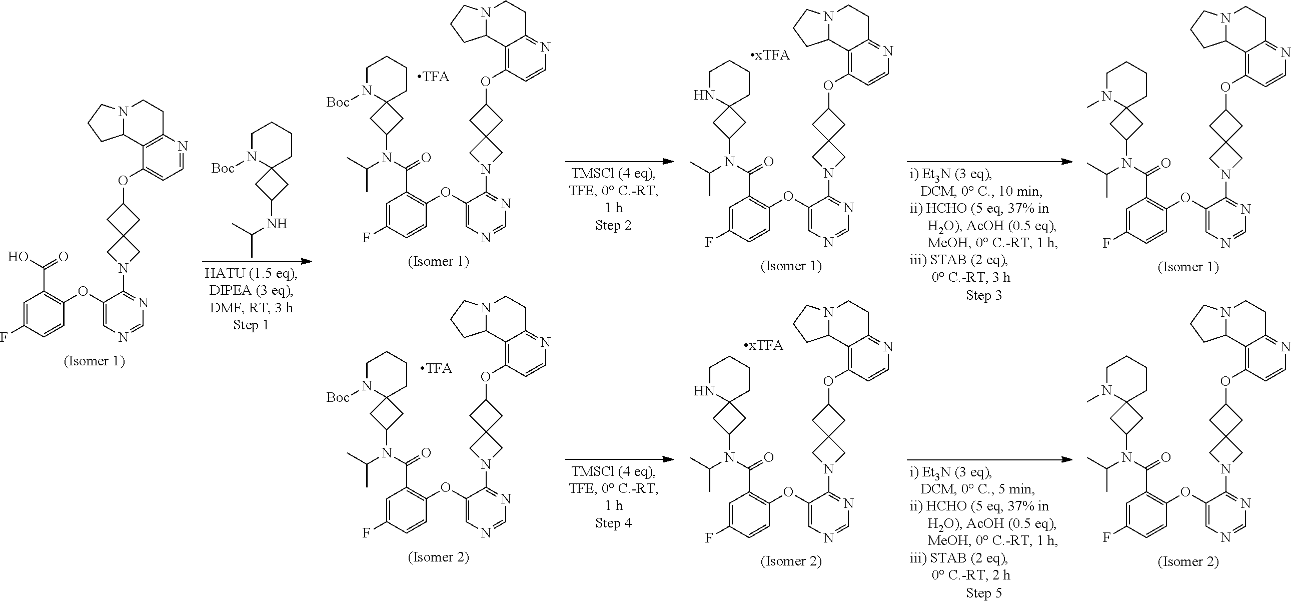

### Step 1. (D1))-tert-Butyl 2-(5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamido)-5-azaspiro[3.5]nonane-5-carboxylate 2,2,2-trifluoroacetate and (D2)-tert-butyl 2-(5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropylbenzamido)-5-azaspiro[3.5]nonane-5-carboxylate 2,2,2-trifluoroacetate

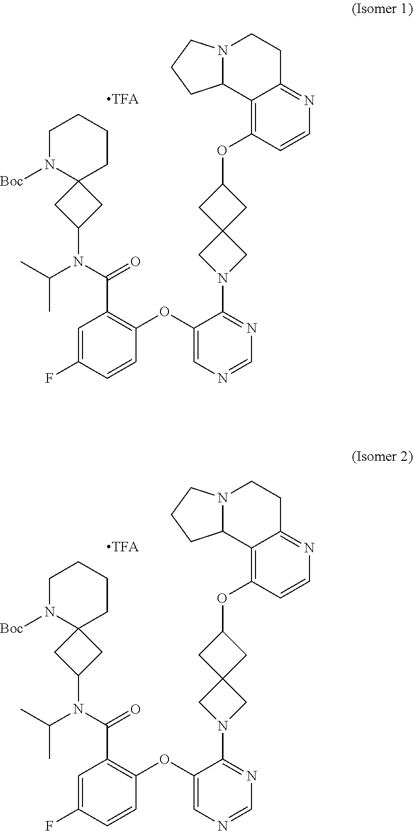 [SMILES: CC(C)N(C(=O)c1cc(F)ccc1Oc1cncnc1N1CC2(CC(Oc3ccnc4c3C3CCCN3CC4)C2)C1)C1CC2(CCCCN2C(=O)OC(C)(C)C)C1 ; CC(C)N(C(=O)c1cc(F)ccc1Oc1cncnc1N1CC2(CC(Oc3ccnc4c3C3CCCN3CC4)C2)C1)C1CC2(CCCCN2C(=O)OC(C)(C)C)C1]

This compound was synthesized following the procedure described for the synthesis of Examples 211 and 212, step 1, starting with 1 equivalent of (E1)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)benzoic acid and 1 equivalent of tert-butyl 2-(isopropylamino)-5-azaspiro[3.5]nonane-5-carboxylate, except that 5 equivalents of DIPEA was used. After completion, the reaction was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain a mixture of diastereomers. The mixture of diastereomers was purified by Prep HPLC (Method G) to obtain both isomers.

Isomer 1. (D1)-tert-Butyl 2-(5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamido)-5-azaspiro[3.5]nonane-5-carboxylate 2,2,2-trifluoroacetate.

Yield: 12.97%; LCMS (Method H): Rt=1.83 min; m/z=782.3 [M+H]⁺, 77.80%.

Isomer 2. (D2)-tert-Butyl 2-(5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropylbenzamido)-5-azaspiro[3.5]nonane-5-carboxylate 2,2,2-trifluoroacetate.

Yield: 7.65%; LCMS (Method H): Rt=1.47 min; m/z=782.3 [M+H]⁺, 93.72%.

### Step 2. (D1)-5-Fluoro-2-((4-(6-((5,6,8,9,10,10α-hexahydropyrrolo[2,1-][1,6]naphthyridin-1-yl)oxy)-2-azaspiro[3.3]heptan-2-yl)pyrimidin-5-yl)oxy)-N-isopropyl-N-(5-azaspiro[3.5]nonan-2-yl)benzamide xTFA

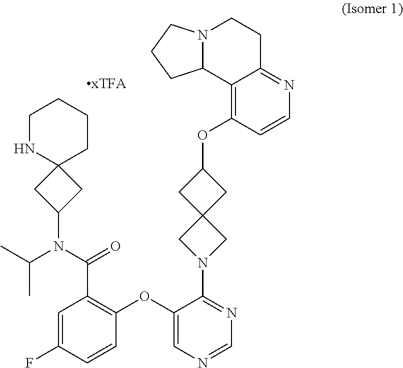 [SMILES: CC(C)N(C(=O)c1cc(F)ccc1Oc1cncnc1N1CC2(CC(Oc3ccnc4c3C3CCCN3CC4)C2)C1)C1CC2(CCCCN2)C1]

This compound was synthesized following the procedure described for the synthesis of Example 20, step 3, starting with 1 equivalent of (D1)-tert-butyl 2-(5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamido)-5-azaspiro[3.5]nonane-5-carboxylate 2,2,2-trifluoroacetate. After completion, the reaction was concentrated under reduced pressure, and reconcentrated from EtOAc to obtain (D1)-5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropyl-N-(5-azaspiro[3.5]nonan-2-yl)benzamide xTFA as a solid. This material was used without further purification.

Yield: Quantitative; LCMS (Method H): Rt=1.04 min; m/z=682.3 [M+H]⁺, 99.02%.

### Step 3. (D1)-5-Fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropyl-N-(5-methyl-5-azaspiro[3.5]nonan-2-yl)benzamide

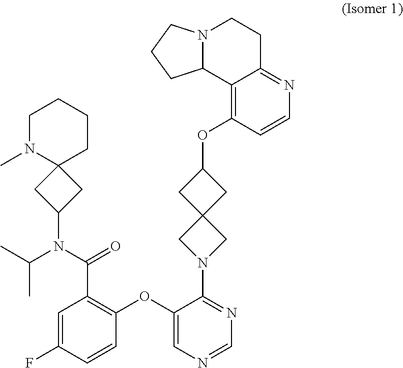 [SMILES: CC(C)N(C(=O)c1cc(F)ccc1Oc1cncnc1N1CC2(CC(Oc3ccnc4c3C3CCCN3CC4)C2)C1)C1CC2(CCCCN2C)C1]

This compound was synthesized following the procedure described for the synthesis of Example 20, step 4, starting with 1 equivalent of (1)₁)-5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropyl-N-(5-azaspiro[3.5]nonan-2-yl)benzamide xTFA, except that 5 equivalents of formaldehyde (37% in H₂O), 3 equivalents of TEA, 0.5 equivalent of AcOH, and 2 equivalents of STAB were used. After completion, the reaction was concentrated under reduced pressure. The crude product was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to obtain (D1)-5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropyl-N-(5-methyl-5-azaspiro[3.5]nonan-2-yl)benzamide as a solid.

Yield: 14.28%; ¹H NMR (400 MHz, DMSO-d₆): δ 8.32-8.23 (m, 1H), 8.17 (d, J=5.7 Hz, 1H), 7.83-7.64 (m, 1H), 7.31-7.02 (m, 3H), 6.71-6.59 (m, 1H), 4.75 (quin, J=6.5 Hz, 1H), 4.37-4.12 (m, 4H), 3.80-3.54 (m, 3H), 3.15-3.05 (m, 1H), 3.01-2.88 (m, 3H), 2.88-2.73 (m, 4H), 2.66-2.61 (m, 2H), 2.43-2.36 (m, 1H), 2.31-2.01 (m, 7H), 1.86-1.55 (m, 5H), 1.52-1.23 (m, 9H), 1.13-0.95 (m, 3H); LCMS (Method H): Rt=1.03 min, m/z=696.4 [M+H]⁺; HPLC (Method G): Rt=6.49 min, 99.74%; Chiral SFC (Method C): Rt=7.07 min, 100%.

### Step 4. (D2)-5-Fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropyl-N-(5-azaspiro[3.5]nonan-2-yl)benzamide xTFA

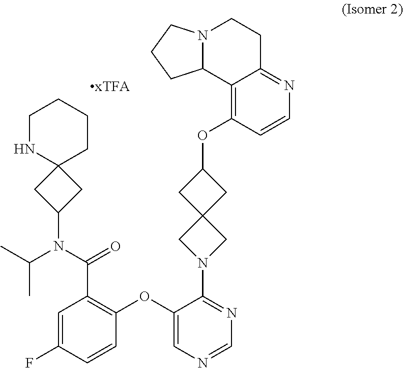 [SMILES: CC(C)N(C(=O)c1cc(F)ccc1Oc1cncnc1N1CC2(CC(Oc3ccnc4c3C3CCCN3CC4)C2)C1)C1CC2(CCCCN2)C1]

This compound was synthesized following the procedure described for the synthesis of Example 20, step 3, starting with 1 equivalent of (D2)-tert-butyl 2-(5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropylbenzamido)-5-azaspiro[3.5]nonane-5-carboxylate 2,2,2-trifluoroacetate. After completion, the reaction was concentrated under reduced pressure, and reconcentrated from EtOAc to obtain crude (D2)-5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropyl-N-(5-azaspiro[3.5]nonan-2-yl)benzamide xTFA as a solid. This material was used without further purification.

Yield: Quantitative; LCMS (Method H): Rt=1.02 min; m/z=682.4 [M+H]⁺, 99.74%.

### Step 5. (D2)-5-Fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropyl-N-(5-methyl-5-azaspiro[3.5]nonan-2-yl)benzamide

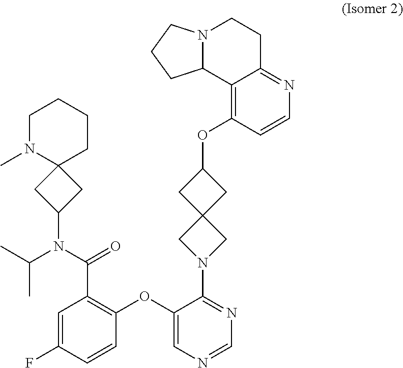 [SMILES: CC(C)N(C(=O)c1cc(F)ccc1Oc1cncnc1N1CC2(CC(Oc3ccnc4c3C3CCCN3CC4)C2)C1)C1CC2(CCCCN2C)C1]

This compound was synthesized following the procedure described for the synthesis of Example 20, step 4, starting with 1 equivalent of (D2)-5-fluoro-2-((4-(6-((5,6,8,9,10,10a-hexahydropyrrolo[2,1-f][1,6]naphthyridin-1-yl)oxy)-2-azaspiro[3.3]heptan-2-yl)pyrimidin-5-yl)oxy)-N-isopropyl-N-(5-azaspiro[3.5]nonan-2-yl)benzamide xTFA, except that 5 equivalents of formaldehyde (37% in H₂O), 3 equivalents of TEA, 0.5 equivalent of AcOH, and 2 equivalents of STAB were used. After completion, the reaction was concentrated under reduced pressure. After work-up, the crude product was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to obtain (D2)-5-fluoro-2-((4-(6-((5,6,8,9,10,10α-hexahydropyrrolo[2,1-f][1,6]naphthyridin-1-yl)oxy)-2-azaspiro[3.3]heptan-2-yl)pyrimidin-5-yl)oxy)-N-isopropyl-N-(5-methyl-5-azaspiro[3.5]nonan-2-yl)benzamide as a solid.

Yield: 14.11%; ¹H NMR (400 MHz, DMSO-d₆): δ 8.30-8.23 (m, 1H), 8.17 (d, J=5.5 Hz, 1H), 7.81-7.53 (m, 1H), 7.33-7.06 (m, 3H), 6.69-6.62 (m, 1H), 4.79-4.70 (m, 1H), 4.37-4.10 (m, 4H), 3.98-3.60 (m, 3H), 3.00-2.88 (m, 2H), 2.87-2.69 (m, 4H), 2.67-2.60 (m, 2H), 2.43-2.38 (m, 1H), 2.31-2.18 (m, 7H), 2.05-1.96 (m, 1H), 1.85-1.64 (m, 4H), 1.54-1.23 (m, 9H), 1.12-0.90 (m, 3H), two protons merged with solvent peaks; LCMS (Method H): Rt=1.02 min, m/z=696.4 [M+H]⁺; HPLC (Method G): Rt=6.40 min, 98.30%; Chiral SFC (Method Y): Rt=7.30 min, 93.59%.

### Example 221. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-6-methyl-6-azaspiro[3.5]nonan-2-yl)benzamide (Compound No. 221) (Method 1)

### Example 222. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2r,4s)-6-methyl-6-azaspiro[3.5]nonan-2-yl)benzamide (Compound No. 222) (Method 1)

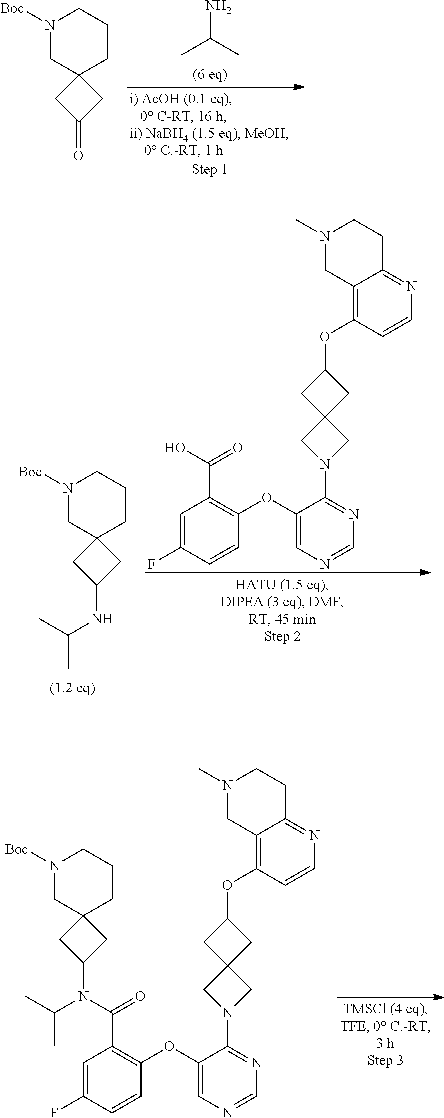

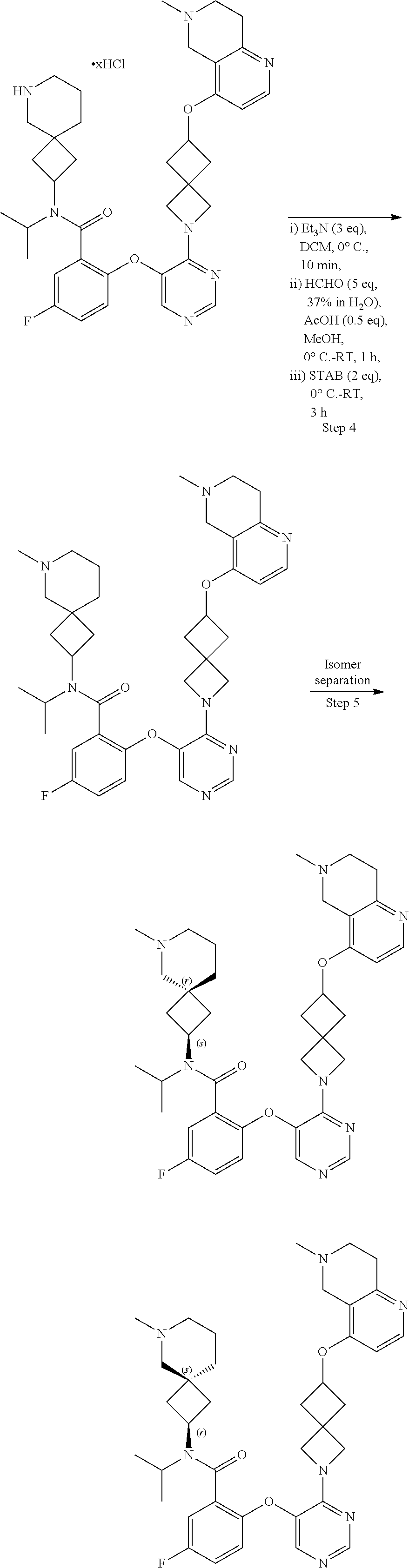

### Step 1. tert-Butyl 2-(isopropylamino)-6-azaspiro[3.5]nonane-6-carboxylate

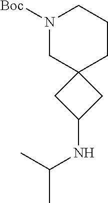 [SMILES: CC(C)NC1CC2(CCCN(C(=O)OC(C)(C)C)C2)C1]

This compound was synthesized following the procedure described for the synthesis of Intermediate 51, starting with 1 equivalent of tert-butyl 2-oxo-6-azaspiro[3.5]nonane-6-carboxylate. After completion, the solvent was concentrated under reduced pressure. The residue was diluted with water and the mixture was extracted with EtOAc. The combined organic layer was dried over sodium sulfate and filtered, and the filtrate was concentrated under reduced pressure to obtain crude tert-butyl 2-(isopropylamino)-6-azaspiro[3.5]nonane-6-carboxylate as a gummy liquid.

Yield: 88%; ¹H NMR (400 MHz, DMSO-d₆): δ3.25-3.14 (m, 5H), 2.75-2.63 (m, 1H), 2.03-1.96 (m, 1H), 1.94-1.86 (in, 1H), 1.56-1.42 (m, 4H), 1.40 (s, 9H), 1.35-1.23 (m, 2H), 0.92 (d, J=6.2 Hz, 6H), three protons merged with solvent peaks; LCMS (Method B): Rt=1.05 min, m/z=283.2 [M+H]⁺; HPLC (Method C): Rt=8.13-8.28 min, 97.26%.

### Step 2. tert-Butyl 2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-6-azaspiro[3.5]nonane-6-carboxylate

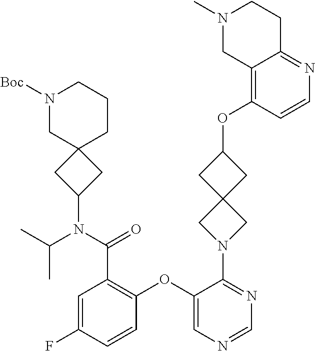 [SMILES: CC(C)N(C(=O)c1cc(F)ccc1Oc1cncnc1N1CC2(CC(Oc3ccnc4c3CN(C)CC4)C2)C1)C1CC2(CCCN(C(=O)OC(C)(C)C)C2)C1]

This compound was synthesized following the procedure described for the synthesis of Examples 211 and 212, step 1, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.2 equivalents of tert-butyl 2-(isopropylamino)-6-azaspiro[3.5]nonane-6-carboxylate. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain a mixture of diastereomers, (D1) tert-butyl 2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-6-azaspiro[3.5]nonane-6-carboxylate and (D2) tert-butyl 2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-6-azaspiro[3.5]nonane-6-carboxylate as a solid.

Yield: 81%; ¹H NMR (400 MHz, DMSO-d₆): δ 8.31-8.22 (m, 1H), 8.20-8.15 (m, 1H), 7.81-7.57 (m, 1H), 7.31-7.01 (m, 3H), 6.69-6.60 (m, 1H), 4.74 (quin, J=6.6 Hz, 1H), 4.35-4.16 (m, 3H), 4.15-4.09 (m, 1H), 3.95-3.63 (m, 2H), 3.36 (br s, 2H), 3.30-3.19 (m, 2H), 2.84-2.74 (m, 5H), 2.63 (br t, J=5.8 Hz, 2H), 2.36 (br s, 3H), 2.30-2.20 (m, 2H), 1.93-1.58 (m, 4H), 1.50-1.23 (m, 16H), 1.12-0.92 (m, 4H); LCMS (Method A): Rt=2.28 min, m/z=756.2 [M+H]⁺; HPLC (Method G): Rt=8.17 min, 99.21%.

### Step 3. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(6-azaspiro[3.5]nonan-2-yl)benzamide xHCl

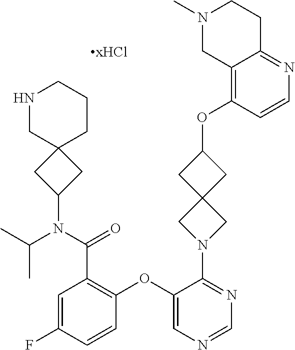 [SMILES: CC(C)N(C(=O)c1cc(F)ccc1Oc1cncnc1N1CC2(CC(Oc3ccnc4c3CN(C)CC4)C2)C1)C1CC2(CCCNC2)C1]

This compound was synthesized following the procedure described for the synthesis of Example 20, step 3, starting with 1 equivalent of a mixture of diastereomers of tert-butyl 2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-6-azaspiro[3.5]nonane-6-carboxylate. After completion, the reaction was concentrated under reduced pressure, and the residue was triturated with EtOAc. The organic layer was decanted and the solid was dried under reduced pressure to obtain crude mixture of diastereomers of 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(6-azaspiro[3.5]nonan-2-yl)benzamide xHCl as a solid.

Yield: Quantitative; LCMS (Method A): Rt=1.92 min, m/z=656.3 [M+H]⁺, 94.32%.

### Step 4. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(6-methyl-6-azaspiro[3.5]nonan-2-yl)benzamide

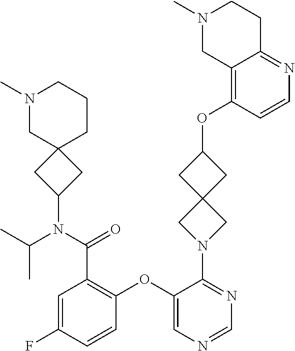 [SMILES: CC(C)N(C(=O)c1cc(F)ccc1Oc1cncnc1N1CC2(CC(Oc3ccnc4c3CN(C)CC4)C2)C1)C1CC2(CCCN(C)C2)C1]

This compound was synthesized following the procedure described for the synthesis of Example 20, step 4, starting with 1 equivalent of 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(6-azaspiro[3.5]nonan-2-yl)benzamide xHCl, except that 5 equivalents of formaldehyde (37%in H₂O), 3 equivalents of TEA, 0.5 equivalent of AcOH, and 2 equivalents of STAB were used. After completion, the reaction was concentrated under reduced pressure. After completion, the reaction was concentrated under reduced pressure. The crude product was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure and lyophilized to afford 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(6-methyl-6-azaspiro[3.5]nonan-2-yl)benzamide as a solid.

Yield: 69.5%; ¹H NMR (400 MHz, DMSO-d₆): δ 8.30-8.25 (m, 1H), 8.18 (br d, J=5.5 Hz, 1H), 7.79-7.65 (m, 1H), 7.31-7.03 (m, 3H), 6.68-6.60 (m, 1H), 4.75 (quin, J=6.4 Hz, 1H), 4.35-4.09 (m, 4H), 4.06-3.83 (m, 1H), 3.80-3.63 (m, 1H), 3.36 (s, 2H), 2.87-2.74 (m, 5H), 2.63 (br t, J=5.6 Hz, 2H), 2.36 (s, 3H), 2.32-2.19 (m, 3H), 2.18-1.96 (m, 6H), 1.93-1.72 (m, 3H), 1.63-1.15 (m, 7H), 1.10-0.93 (m, 3H); LCMS (Method A): Rt=1.92-2.04 min, m/z=670.4 [M+H]⁺; HPLC (Method G): Rt=6.32-6.38 min, 99.55%; Chiral SFC (Method P): Peak 1; Rt=3.50 min, 39.27%; Peak 2; Rt=4.36 min, 60.73%.

### Step 5. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-6-methyl-6-azaspiro[3.5]nonan-2-yl)benzamide and 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2r,4s)-6-methyl-6-azaspiro[3.5]nonan-2-yl)benzamide

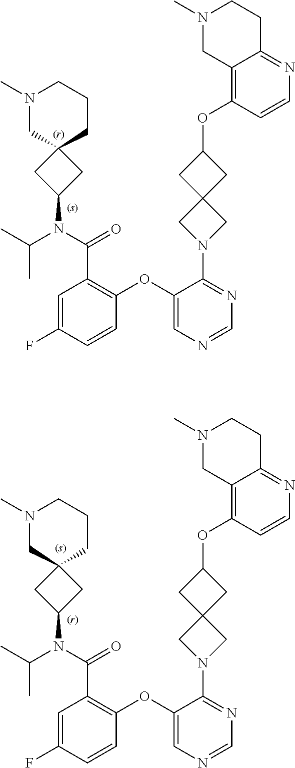 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCN(C)C2)C1 ; CC(C)N(C(=O)c1cc(F)ccc1Oc1cncnc1N1CC2(CC(Oc3ccnc4c3CN(C)CC4)C2)C1)[C@H]1C[C@]2(CCCN(C)C2)C1]

The mixture of diastereomers of 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(6-methyl-6-azaspiro[3.5]nonan-2-yl)benzamide (420 mg, 0.627 mmol) was purified by chiral Prep SFC (Method AE). The pure fractions were concentrated under reduced pressure to obtain both isomers. Each isomer was then worked up further as follows: The individual isomer was diluted with saturated sodium bicarbonate (10 mL) and the mixture was extracted with 10% MeOH in DCM (2×10 mL). The combined organic layer was dried over anhydrous sodium sulfate and filtered, and the filtrate was concentrated under reduced pressure. The residue was reconcentrated from ACN (2×5 mL) and lyophilized to obtain the individual isomer. Subsequently, peak 1 was re-purified by chiral Prep SFC (Method AF).

Isomer 1. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-6-methyl-6-azaspiro[3.5]nonan-2-yl)benzamide (73 mg, 17.35% yield) as a solid. ¹H NMR (400 MHz, DMSO-d₆): δ 8.27 (s, 1H), 8.18 (d, J=5.6 Hz, 1H), 7.80-7.64 (m, 1H), 7.31-7.01 (m, 3H), 6.64 (br d, J=5.5 Hz, 1H), 4.75 (quin, J=6.6 Hz, 1H), 4.35-4.08 (m, 4H), 4.07-3.82 (m, 1H), 3.80-3.62 (m, 1H), 3.36 (s, 2H), 2.84-2.75 (m, 5H), 2.63 (br t, J=5.8 Hz, 2H), 2.36 (s, 3H), 2.30-2.20 (m, 3H), 2.18-1.72 (m, 9H), 1.52-1.25 (m, 7H), 1.10-0.92 (m, 3H); LCMS (Method H): Rt=0.99 min, m/z=670.3 [M+H]⁺, HPLC (Method C): Rt=8.77 min, 99.62%; Chiral SFC (Method P): Rt=3.19 min, 100%.

Isomer 2. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2r,4s)-6-methyl-6-azaspiro[3.5]nonan-2-yl)benzamide (145 mg, 34.5% yield) as a solid. ¹H NMR (400 MHz, DMSO-d₆): δ 8.30-8.25 (m, 1H), 8.17 (br d, J=5.3 Hz, 1H), 7.79-7.65 (m, 1H), 7.31-7.00 (m, 3H), 6.68-6.60 (m, 1H), 4.75 (quin, J=6.5 Hz, 1H), 4.35-4.09 (m, 4H), 4.03-3.85 (m, 1H), 3.78-3.62 (m, 1H), 3.36 (s, 2H), 2.88-2.75 (m, 5H), 2.64-2.60 (m, 2H), 2.36 (s, 3H), 2.31-2.19 (m, 3H), 2.17-2.01 (m, 6H), 1.86-1.54 (m, 4H), 1.50-1.38 (m, 3H), 1.37-1.16 (m, 3H), 1.08-0.93 (m, 4H); LCMS (Method H): Rt=0.99 min, m/z=670.4 [M+H]⁺, HPLC (Method G): Rt=6.39 min, 99.85%; Chiral SFC (Method P): Rt=4.30 min, 99.61%.

### Example 221. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-6-methyl-6-azaspiro[3.5]nonan-2-yl)benzamide (Compound No. 221) (Method 2)

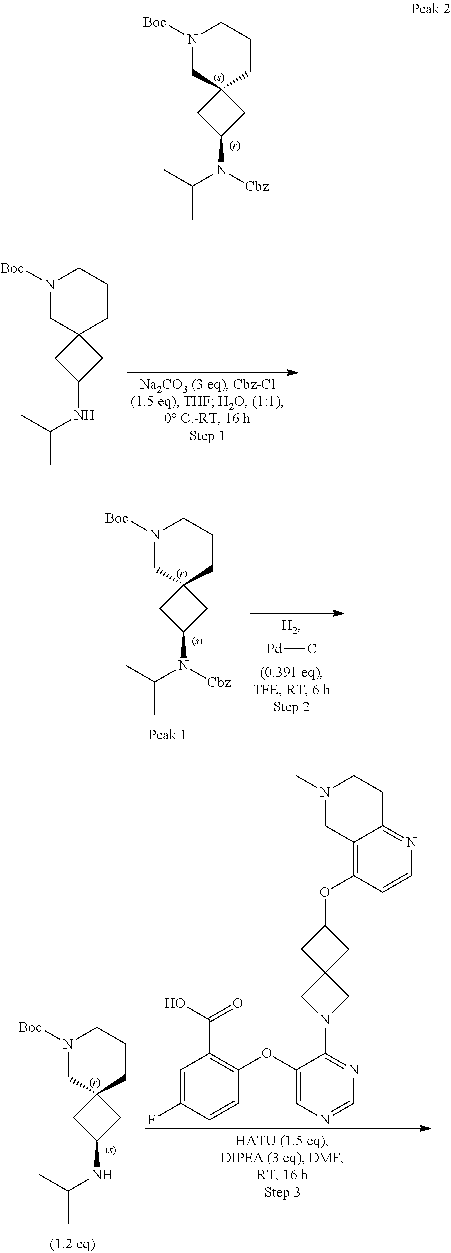

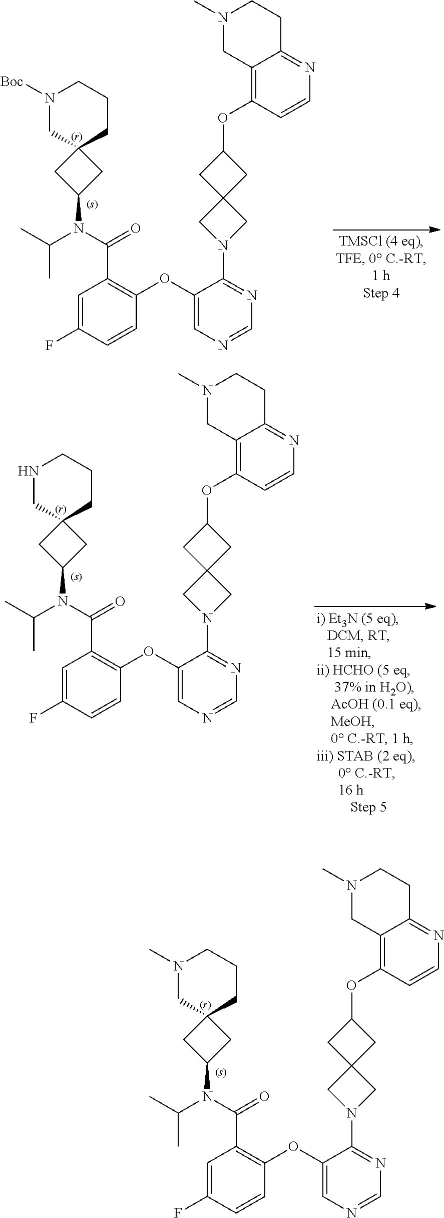

### Step 1. tert-Butyl(2s,4r)-2-(((benzyloxy)carbonyl)(isopropyl)amino)-6-azaspiro[3.5]nonane-6-carboxylate and tert-butyl(2r,4s)-2-(((benzyloxy)carbonyl)(isopropyl)amino)-6-azaspiro[3.5]nonane-6-carboxylate

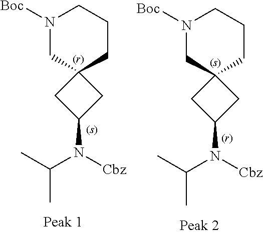 [SMILES: CC(C)N(C(=O)OCc1ccccc1)[C@H]1C[C@@]2(CCCN(C(=O)OC(C)(C)C)C2)C1 ; CC(C)N(C(=O)OCc1ccccc1)[C@H]1C[C@]2(CCCN(C(=O)OC(C)(C)C)C2)C1]

To a stirred solution of tert-butyl 2-(isopropylamino)-6-azaspiro[3.5]nonane-6-carboxylate (1.1 g, 3.89 mmol) in 1:1 THF:H₂O (10 mL:10 mL), sodium carbonate (1.238 g, 11.68 mmol) and Cbz-Cl (0.834 mL, 5.84 mmol) were added at 0° C. The reaction was stirred at RT for 18 h under a nitrogen atmosphere, and monitored by LCMS and TLC (30% EtOAc in hexane). After completion, the reaction was quenched with water (10 mL), and the mixture was extracted with DCM (2×10 mL). The combined organic layer was dried over anhydrous sodium sulfate and filtered, and the filtrate was concentrated under reduced pressure. The crude product was purified by silica gel flash column chromatography using EtOAc in hexane (product eluted at 20% EtOAc in hexane) to obtain a mixture of products. This mixture (1.6 g) was purified by chiral Prep SFC (Method AG) to obtain both isomers.

Peak 1: tert-Butyl(2s,4r)-2-(((benzyloxy)carbonyl)(isopropyl)amino)-6-azaspiro[3.5]nonane-6-carboxylate (490 mg, 29.5% yield) as a sticky liquid. ¹H NMR (400 MHz, DMSO-d₆): δ 7.42-7.27 (m, 5H), 5.08 (s, 2H), 4.13-3.95 (m, 2H), 3.26 (br s, 2H), 3.21 (br t, J=5.0 Hz, 2H), 2.12-2.01 (m, 2H), 1.93-1.82 (m, 2H), 1.39 (s, 9H), 1.32-1.22 (m, 2H), 1.13 (d, J=6.8 Hz, 6H), two protons merged with solvent peaks; LCMS (Method B): Rt=2.45 min, m/z=317.2 [M+H-Boc]⁺; HPLC (Method C): Rt=13.21 min, 97.76%. Chiral SFC (Method AAU): Rt=4.62 min, 100%.

Peak 2: tert-Butyl(2r,4s)-2-(((benzyloxy)carbonyl)(isopropyl)amino)-6-azaspiro[3.5]nonane-6-carboxylate (700 mg, 43% yield) as a solid. ¹H NMR (400 MHz, DMSO-d₆): δ 7.44-7.27 (m, 5H), 5.10 (s, 2H), 4.17-3.94 (m, 2H), 3.30-3.24 (m, 2H), 3.21 (br t, J=5.2 Hz, 2H), 2.31-2.16 (m, 2H), 1.89-1.75 (m, 2H), 1.66-1.56 (m, 2H), 1.45-1.39 (m, 2H), 1.38 (s, 9H), 1.13 (d, J=6.7 Hz, 6H); LCMS (Method B): Rt=2.47 min, m/z=317.2 [M+H-Boc]⁺; HPLC (Method C): Rt=13.28 min, 99.69%. Chiral SFC (Method AAU): Rt=5.25 min, 100%.

### Step 2. tert-Butyl (2s,4r)-2-(isopropylamino)-6-azaspiro[3.5]nonane-6-carboxylate

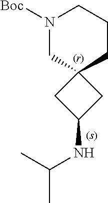 [SMILES: CC(C)N[C@H]1C[C@@]2(CCCN(C(=O)OC(C)(C)C)C2)C1]

To a stirred solution of tert-butyl(2s,4r)-2-(((benzyloxy)carbonyl)(isopropyl)amino)-6-azaspiro[3.5]nonane-6-carboxylate (450 mg, 1.080 mmol) in TFE (8 mL), Pd—C (450 mg, 0.423 mmol) was added at RT. The reaction was stirred at RT for 6 h under a hydrogen atmosphere (balloon pressure), and monitored by TLC (10% MeOH in DCM) and LCMS. After completion, the reaction was filtered through a Celite® pad, and the pad was washed with MeOH (2×30 mL). The filtrate was concentrated under reduced pressure to afford crude tert-butyl(2s,4r)-2-(isopropylamino)-6-azaspiro[3.5]nonane-6-carboxylate (380 mg, quantitative yield) as a sticky liquid. This material was used without further purification. LCMS (Method B): Rt=1.07 min, m/z=283.2 [M+H]⁺, 90.37%.

### Step 3. tert-Butyl(2s,4r)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-6-azaspiro[3.5]nonane-6-carboxylate

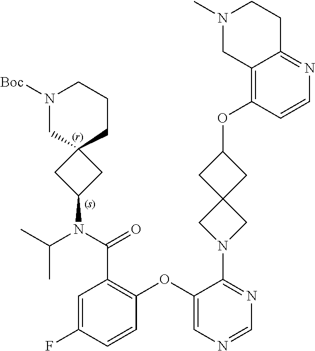 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCN(C(=O)OC(C)(C)C)C2)C1]

In a dried, 50 mL round bottom flask under a nitrogen atmosphere, 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid (550 mg, 1.119 mmol) and tert-butyl(2s,4r)-2-(isopropylamino)-6-azaspiro[3.5]nonane-6-carboxylate (379 mg, 1.343 mmol) were dissolved in DMF (5 mL). To this reaction mixture, DIPEA (0.586 mL, 3.36 mmol) and HATU (638 mg, 1.678 mmol) were added at 0° C. The reaction was stirred at RT for 16 h, and monitored by LCMS and TLC (10% MeOH in DCM). After completion, the reaction was quenched with water (40 mL) and the mixture was extracted with EtOAc (3×20 mL). The combined organic layer was washed with brine (30 mL), dried over anhydrous sodium sulfate and filtered, and the filtrate was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl(2s,4r)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-6-azaspiro[3.5]nonane-6-carboxylate (330 mg, 34.5% yield) as a solid. LCMS (Method B): Rt=1.43 min, m/z=756.3 [M+H]⁺, 88.33%.

### Step 4. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-6-azaspiro[3.5]nonan-2-yl)benzamide xHCl

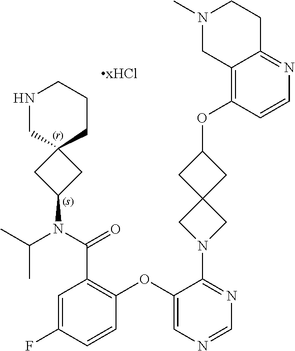 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCNC2)C1]

In a 25 mL round bottom flask, tert-butyl(2s,4r)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-6-azaspiro[3.5]nonane-6-carboxylate (330 mg, 0.437 mmol) was dissolved in TFE (5 mL). To this solution, TMSCl (0.223 mL, 1.746 mmol) as added at 0° C. The reaction was stirred at RT for 1 h, and monitored by LCMS and TLC (10% MeOH in DCM). After completion, the reaction was concentrated under reduced pressure to afford crude 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-6-azaspiro[3.5]nonan-2-yl)benzamide xHCl (0.37 g, quantitative yield) as a gum. This material was used without further purification. LCMS (Method B): Rt=0.82 min, m/z=656.4 [M+H]⁺, 93.19%.

### Step 5. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-6-methyl-6-azaspiro[3.5]nonan-2-yl)benzamide

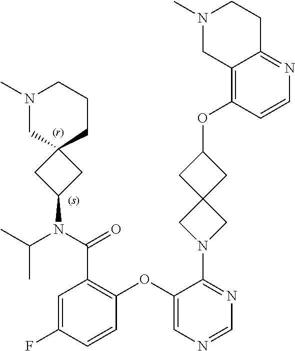 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCN(C)C2)C1]

To a stirred solution of 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-6-azaspiro[3.5]nonan-2-yl)benzamide xHCl (0.37 g, 0.534 mmol) in DCM (5 mL), TEA (0.372 mL, 2.67 mmol) was added at RT. The reaction was stirred at RT for 15 min, then was concentrated under reduced pressure. The residue was dissolved in MeOH (5 mL), and acetic acid (3.06 μL, 0.053 mmol) and formaldehyde (0.074 mL, 2.67 mmol, 37% in H₂O) were added at RT. The reaction was stirred at RT for 1 h, then sodium triacetoxyborohydride (0.227 g, 1.069 mmol) was added at 0° C. The reaction was stirred at RT for 16 h, and monitored by LCMS. After completion, the reaction was concentrated under reduced pressure. The crude was dissolved in 10% MeOH-DCM (20 mL) and washed with sodium bicarbonate solution (15 mL). The combined organic layer was dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 0.1% NH₄HCO₃ in water, B: ACN). The fractions were concentrated under reduced pressure and lyophilized to afford 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-6-methyl-6-azaspiro[3.5]nonan-2-yl)benzamide (93 mg, 25.8% yield) as a solid. ¹H NMR (400 MHz, DMSO-d₆): δ 8.27 (s, 1H), 8.18 (d, J=5.7 Hz, 1H), 7.79-7.67 (m, 1H), 7.31-7.04 (m, 3H), 6.64 (br d, J=5.5 Hz, 1H), 4.75 (quin, J=6.4 Hz, 1H), 4.37-4.08 (m, 4H), 4.07-3.82 (m, 1H), 3.80-3.61 (m, 1H), 3.36 (s, 2H), 2.83-2.75 (m, 5H), 2.63 (br t, J=5.7 Hz, 2H), 2.36 (s, 3H), 2.30-2.20 (m, 3H), 2.18-1.71 (m, 9H), 1.53-1.23 (m, 7H), 1.10-0.91 (m, 3H); LCMS (Method B): Rt=0.87 min, m/z=670.3 [M+H]⁺; HPLC (Method G): Rt=6.28 min, 99.41%; Chiral SFC Chiral SFC (Method AAA): Rt=2.84 min, 100%.

### Example 223. 5-Fluoro-N-isopropyl-N-(7-methyl-7-azaspiro[3.5]nonan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (Compound No. 223)

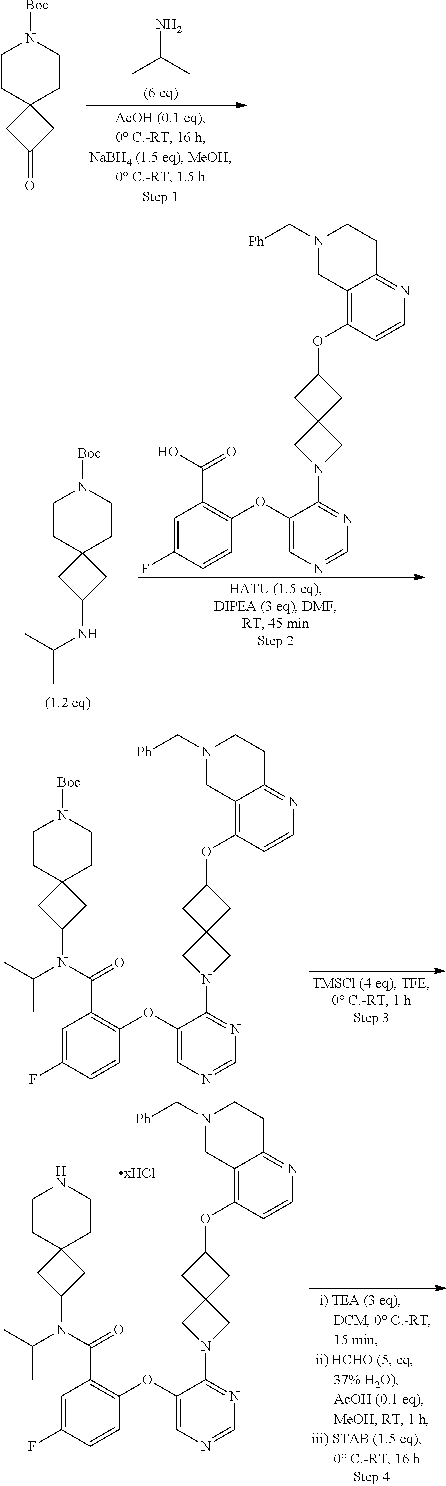

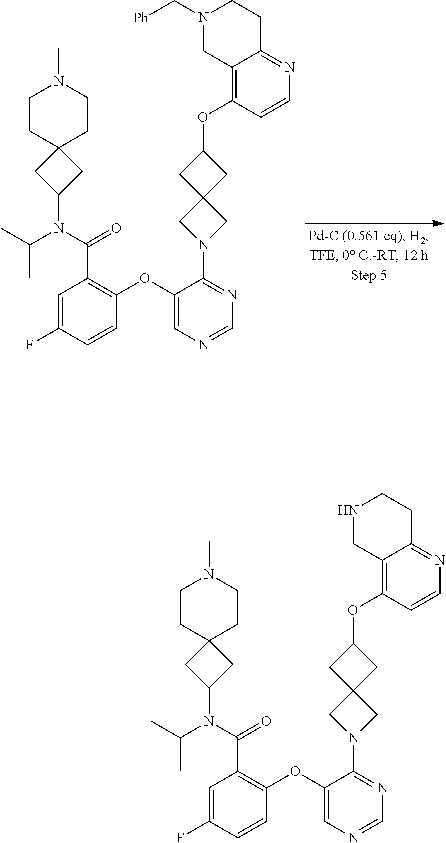 [SMILES: CC(C)N(C(=O)c1cc(F)ccc1Oc1cncnc1N1CC2(CC(Oc3ccnc4c3CN(Cc3ccccc3)CC4)C2)C1)C1CC2(CCN(C)CC2)C1 ; CC(C)N(C(=O)c1cc(F)ccc1Oc1cncnc1N1CC2(CC(Oc3ccnc4c3CNCC4)C2)C1)C1CC2(CCN(C)CC2)C1]

### Step 1. tert-Butyl 2-(isopropylamino)-7-azaspiro[3.5]nonane-7-carboxylate

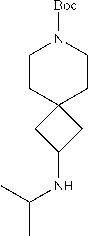 [SMILES: CC(C)NC1CC2(CCN(C(=O)OC(C)(C)C)CC2)C1]

This compound was synthesized following the procedure described for the synthesis of Intermediate 51, starting with 1 equivalent of tert-butyl 2-oxo-7-azaspiro[3.5]nonane-7-carboxylate. After completion, the reaction was concentrated under reduced pressure. The residue was diluted with water and the mixture was extracted with EtOAc. The combined organic layer was dried over sodium sulfate and filtered, and the filtrate was concentrated under reduced pressure to obtain crude tert-butyl 2-(isopropylamino)-7-azaspiro[3.5]nonane-7-carboxylate as a gummy liquid.

Yield: 88.51%; ¹H NMR (400 MHz, DMSO-d₆): δ 3.27-3.14 (m, 5H), 2.68 (spt, J=6.2 Hz, 1H), 2.10-2.03 (m, 2H), 1.46-1.35 (m, 15H), 0.92 (d, J=6.3 Hz, 6H), one proton merged with solvent peaks; LCMS (Method A): Rt=1.65 min, m/z=283.3 [M+H]⁺, 98.53%.

### Step 2. tert-Butyl 2-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-7-azaspiro[3.5]nonane-7-carboxylate

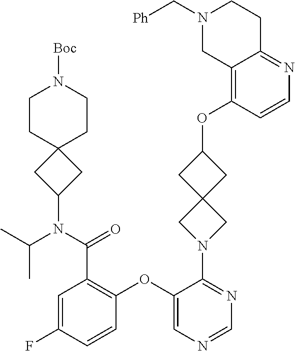 [SMILES: CC(C)N(C(=O)c1cc(F)ccc1Oc1cncnc1N1CC2(CC(Oc3ccnc4c3CN(Cc3ccccc3)CC4)C2)C1)C1CC2(CCN(C(=O)OC(C)(C)C)CC2)C1]

This compound was synthesized following the procedure described for the synthesis of Examples 211 and 212, step 1, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluorobenzoic acid and 1.2 equivalents of tert-butyl 2-(isopropylamino)-7-azaspiro[3.5]nonane-7-carboxylate. After work-up, the crude was purified by reverse phase chromatography using a SiliCycle SiliaSep C18 column (120 g) on Biotage isolera one (Mobile phase A: 10 mM NH₄HCO₃ in water, B: ACN) to obtain tert-butyl 2-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-7-azaspiro[3.5]nonane-7-carboxylate as a solid.

Yield: 64.6%; ¹H NMR (400 MHz, DMSO-d₆): δ 8.30-8.25 (m, 1H), 8.21-8.16 (m, 1H), 7.78-7.66 (m, 1H), 7.35 (d, J=4.2 Hz, 4H), 7.30-7.05 (m, 4H), 6.68-6.61 (m, 1H), 4.71 (quin, J=6.5 Hz, 1H), 4.35-3.87 (m, 5H), 3.79-3.62 (m, 3H), 3.47 (s, 2H), 3.30-3.07 (m, 4H), 2.93-2.65 (m, 7H), 2.26-2.15 (m, 2H), 1.98-1.67 (m, 3H), 1.55 (br d, J=4.7 Hz, 2H), 1.47-1.26 (m, 13H), 1.12-0.92 (m, 4H); LCMS (Method A): Rt=2.55 min, m/z=832.3 [M+H]⁺, 99.74%.

### Step 3. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(7-azaspiro[3.5]nonan-2-yl)benzamide xHCl

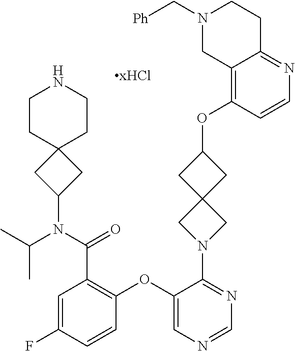 [SMILES: CC(C)N(C(=O)c1cc(F)ccc1Oc1cncnc1N1CC2(CC(Oc3ccnc4c3CN(Cc3ccccc3)CC4)C2)C1)C1CC2(CCNCC2)C1]

This compound was synthesized following the procedure described for the synthesis of Example 20, step 3, starting with 1 equivalent of tert-butyl 2-(2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropylbenzamido)-7-azaspiro[3.5]nonane-7-carboxylate. After completion, the reaction was concentrated under reduced pressure to obtain crude 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(7-azaspiro[3.5]nonan-2-yl)benzamide xHCl as a solid. This material was used without further purification.

Yield: Quantitative; LCMS (Method A): Rt=2.48 min, m/z=732.4 [M+H]⁺; HPLC (Method C): Rt=9.71 min, 95.59%.

### Step 4. 2-((4-(6-((6-Benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(7-methyl-7-azaspiro[3.5]nonan-2-yl)benzamide

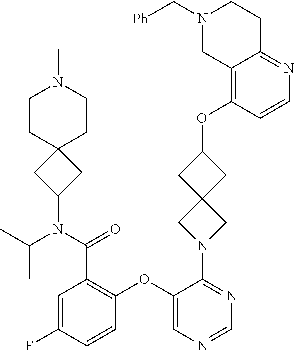 [SMILES: CC(C)N(C(=O)c1cc(F)ccc1Oc1cncnc1N1CC2(CC(Oc3ccnc4c3CN(Cc3ccccc3)CC4)C2)C1)C1CC2(CCN(C)CC2)C1]

This compound was synthesized following the procedure described for the synthesis of Example 20, step 4, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(7-azaspiro[3.5]nonan-2-yl)benzamide xHCl, except that 5 equivalents of formaldehyde (37% in H₂O), 3 equivalents of TEA, 0.1 equivalent of AcOH, and 1.5 equivalents of STAB were used. After completion, the reaction was concentrated under reduced pressure. After work-up, the crude product was purified by reverse phase chromatography using a SiliCycle SiliaSep C18 column (120 g) on Biotage isolera one (Mobile phase A: 10 mM NH₄HCO₃ in water, B: ACN). The pure fractions were concentrated under reduced pressure and lyophilized to afford 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(7-methyl-7-azaspiro[3.5]nonan-2-yl)benzamide as a solid.

Yield: 85%; ¹H NMR (400 MHz, DMSO-d₆): δ 8.32-8.24 (m, 1H), 8.19 (d, J=5.5 Hz, 1H), 7.81-7.61 (m, 1H), 7.35 (d, J=4.3 Hz, 4H), 7.31-7.04 (m, 4H), 6.70-6.59 (m, 1H), 4.72 (quin, J=6.5 Hz, 1H), 4.34-4.08 (m, 4H), 4.03-3.82 (m, 1H), 3.78-3.62 (m, 3H), 3.47 (s, 2H), 2.86-2.60 (m, 7H), 2.28-1.98 (m, 8H), 1.92-1.53 (m, 5H), 1.51-1.27 (m, 5H), 1.12-0.90 (m, 4H); LCMS (Method H): Rt=1.13 min, m/z=746.4 [M+H]⁺; HPLC (Method C): Rt=10.02 min, 99.46%.

### Step 5. 5-Fluoro-N-isopropyl-N-(7-methyl-7-azaspiro[3.5]nonan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide

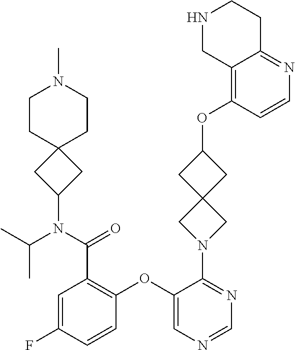 [SMILES: CC(C)N(C(=O)c1cc(F)ccc1Oc1cncnc1N1CC2(CC(Oc3ccnc4c3CNCC4)C2)C1)C1CC2(CCN(C)CC2)C1]

This compound was synthesized following the procedure described for the synthesis of Example 5, step 1, starting with 1 equivalent of 2-((4-(6-((6-benzyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-(7-methyl-7-azaspiro[3.5]nonan-2-yl)benzamide, except that 0.561 equivalent of Pd—C was used. After filtration, the crude product was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure and lyophilized to obtain 5-fluoro-N-isopropyl-N-(7-methyl-7-azaspiro[3.5]nonan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide.

Yield: 7.65%; ¹H NMR (400 MHz, DMSO-d₆): δ 8.31-8.23 (m, 1H), 8.16 (d, J=5.6 Hz, 1H), 7.79-7.64 (m, 1H), 7.31-7.05 (m, 3H), 6.68-6.60 (m, 1H), 4.73 (quin, J=6.5 Hz, 1H), 4.35-4.10 (m, 4H), 4.04-3.84 (m, 1H), 3.77-3.62 (m, 3H), 2.94 (br t, J=5.6 Hz, 2H), 2.85-2.74 (m, 3H), 2.67 (br t, J=5.0 Hz, 2H), 2.28-2.01 (m, 9H), 1.91-1.56 (m, 5H), 1.51-1.26 (m, 4H), 1.13-0.93 (m, 3H), two protons merged with solvent peaks; LCMS (Method H): Rt=0.98 min, m/z=656.3 [M+H]⁺; HPLC (Method G): Rt=6.31 min, 99.68%.

### Example 224. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2r,4s)-5-methyl-5-azaspiro[3.4]octan-2-yl)benzamide (Compound No. 224)

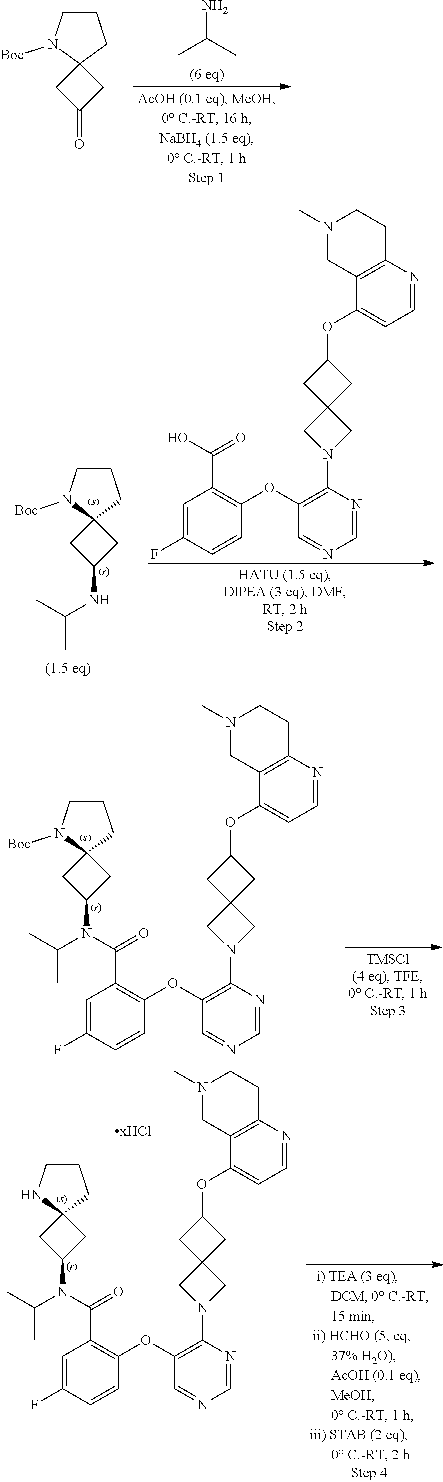

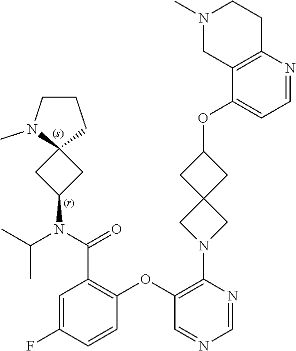

### Step 1. tert-Butyl (2r,4s)-2-(isopropylamino)-5-azaspiro[3.4]octane-5-carboxylate

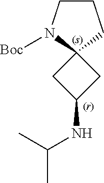 [SMILES: CC(C)N[C@H]1C[C@]2(CCCN2C(=O)OC(C)(C)C)C1]

In 40 mL pressure vial, tert-butyl 2-oxo-5-azaspiro[3.4]octane-5-carboxylate (500 mg, 2.219 mmol) was dissolved in MeOH (10 mL), then propan-2-amine (1.134 mL, 13.32 mmol) and AcOH (0.013 mL, 0.222 mmol) were added at 0° C. The reaction was stirred at RT for 16 h. To this reaction mixture, NaBH₄ (126 mg, 3.33 mmol) was added at 0° C., and the reaction was stirred at 0° C. for 1 h. The reaction progress was monitored by TLC (10% MeOH in DCM). After completion, the reaction was concentrated under reduced pressure. The residue was dissolved in water (10 mL) and the mixture was extracted with 10% MeOH in DCM (3×10 mL). The combined organic layer was dried over sodium sulfate and filtered, and the filtrate was concentrated under reduced pressure to obtain crude tert-butyl (2r,4s)-2-(isopropylamino)-5-azaspiro[3.4]octane-5-carboxylate (610 mg, quantitative yield) as a sticky liquid. This material was used without further purification. ¹H NMR (400 MHz, DMSO-d₆): δ 3.23 (t, J=6.7 Hz, 2H), 3.02-2.87 (m, 1H), 2.77-2.60 (m, 2H), 2.11-1.84 (m, 4H), 1.62 (quin, J=6.7 Hz, 2H), 1.52-1.36 (m, 9H), 0.93 (d, J=6.1 Hz, 6H), three protons merged with solvent peaks; LCMS (Method B): Rt=1.22 min, m/z=269.3 [M+H]⁺, 99.15%.

### Step 2. tert-Butyl(2r,4s)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.4]octane-5-carboxylate

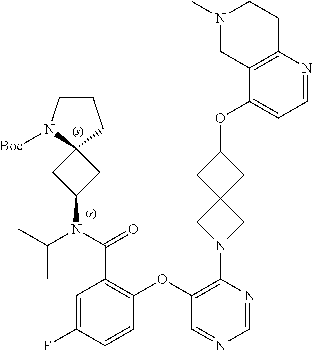 [SMILES: CC(C)N(C(=O)c1cc(F)ccc1Oc1cncnc1N1CC2(CC(Oc3ccnc4c3CN(C)CC4)C2)C1)[C@H]1C[C@]2(CCCN2C(=O)OC(C)(C)C)C1]

This compound was synthesized following the procedure described for the synthesis of Example 1, step 2, starting with 1 equivalent of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid and 1.5 equivalents of tert-butyl(2r,4s)-2-(isopropylamino)-5-azaspiro[3.4]octane-5-carboxylate. After work-up, the crude was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure to obtain tert-butyl(2r,4s)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.4]octane-5-carboxylate as a solid.

Yield: 68.2%; LCMS (Method H): Rt=1.43 min, m/z=742.4 [M+H]⁺, 99.88%.

### Step 3. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2r,4s)-5-azaspiro[3.4]octan-2-yl)benzamide xHCl

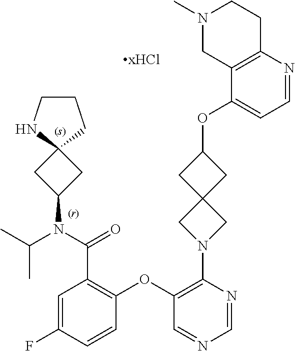 [SMILES: CC(C)N(C(=O)c1cc(F)ccc1Oc1cncnc1N1CC2(CC(Oc3ccnc4c3CN(C)CC4)C2)C1)[C@H]1C[C@]2(CCCN2)C1]

This compound was synthesized following the procedure described for the synthesis of Example 20, step 3, starting with 1 equivalent of tert-butyl(2r,4s)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.4]octane-5-carboxylate. After completion, the reaction was concentrated under reduced pressure. The crude was reconcentrated from EtOAc to afford crude 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2r,4s)-5-azaspiro[3.4]octan-2-yl)benzamide xHCl as a solid. This material was used without further purification.

Yield: Quantitative; LCMS (Method B): Rt=1.00 min, m/z=642.3 [M+H]⁺, 92.50%.

### Step 3. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2r,4s)-5-methyl-5-azaspiro[3.4]octan-2-yl)benzamide

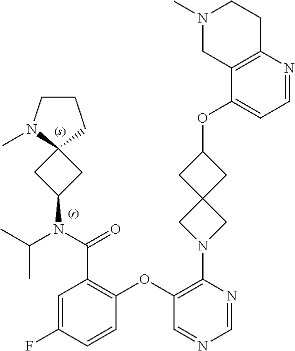 [SMILES: CC(C)N(C(=O)c1cc(F)ccc1Oc1cncnc1N1CC2(CC(Oc3ccnc4c3CN(C)CC4)C2)C1)[C@H]1C[C@]2(CCCN2C)C1]

This compound was synthesized following the procedure described for the synthesis of Example 20, step 4, starting with 1 equivalent of 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2r,4s)-5-azaspiro[3.4]octan-2-yl)benzamide xHCl, except that 3 equivalents of TEA, 5 equivalents of formaldehyde (37% in H₂O), 0.1 equivalent of AcOH, and 2 equivalents of STAB were used. After completion, the reaction was concentrated under reduced pressure. The residue was diluted with water and the mixture was extracted with EtOAc. The combined organic layer was washed with brine, dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2r,4s)-5-methyl-5-azaspiro[3.4]octan-2-yl)benzamide as a solid.

Yield: 57.8%; ¹H NMR (400 MHz, DMSO-d₆): δ 8.31-8.24 (m, 1H), 8.18 (d, J=5.6 Hz, 1H), 7.81-7.64 (m, 1H), 7.32-7.03 (m, 3H), 6.70-6.59 (m, 1H), 4.74 (quin, J=6.6 Hz, 1H), 4.42-4.07 (m, 4H), 3.80-3.58 (m, 2H), 3.36 (s, 2H), 3.23-3.12 (m, 1H), 2.87-2.74 (m, 4H), 2.63 (br t, J=5.9 Hz, 2H), 2.39-2.31 (m, 5H), 2.29-2.16 (m, 4H), 1.87 (br t, J=7.2 Hz, 1H), 1.78-1.52 (m, 5H), 1.49-1.28 (m, 3H), 1.15-0.96 (m, 3H), two protons merged with solvent peaks; LCMS (Method H): Rt=1.00 min, m/z=656.3 [M+H]⁺; HPLC (Method C): Rt=8.35 min, 99.69%; Chiral SFC (Method K): Rt=2.47 min, 99.21%.

### Example 225. 5-Fluoro-N-isopropyl-2-((5-(6-((6-methyl-5,6,7,8-tetrahydro-1,6-naphthyridin-4-yl)oxy)-2-azaspiro[3.3]heptan-2-yl)-1,2,4-triazin-6-yl)oxy)-N-((2s,4r)-5-methyl-5-azaspiro[3.4]octan-2-yl)benzamide (Compound No. 225)

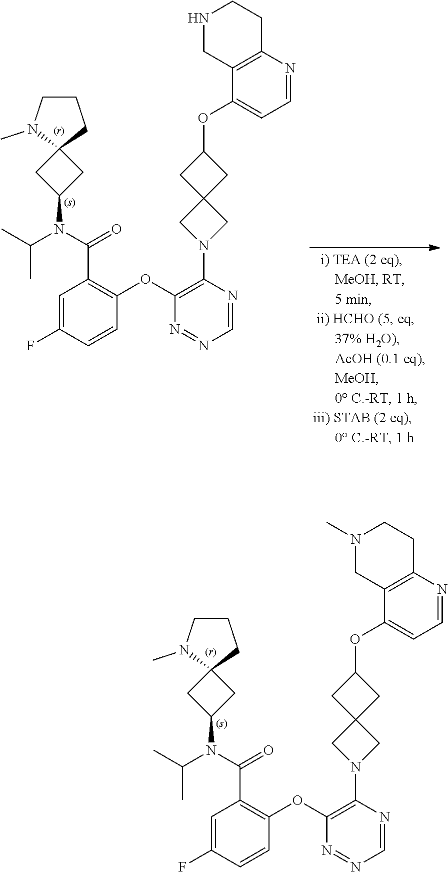 [SMILES: CC(C)N(C(=O)c1cc(F)ccc1Oc1nncnc1N1CC2(CC(Oc3ccnc4c3CNCC4)C2)C1)[C@H]1C[C@@]2(CCCN2C)C1 ; CC(C)N(C(=O)c1cc(F)ccc1Oc1nncnc1N1CC2(CC(Oc3ccnc4c3CN(C)CC4)C2)C1)[C@H]1C[C@@]2(CCCN2C)C1]

This compound was synthesized following the procedure described for the synthesis of Example 20, step 4, starting with 1 equivalent of 5-fluoro-N-isopropyl-N-((2s,4r)-5-methyl-5-azaspiro[3.4]octan-2-yl)-2-((5-(6-((5,6,7,8-tetrahydro-1,6-naphthyridin-4-yl)oxy)-2-azaspiro[3.3]heptan-2-yl)-1,2,4-triazin-6-yl)oxy)benzamide, except that 5 equivalents of formaldehyde, 0.1 equivalent of AcOH, and 2 equivalents of STAB were used, and MeOH was used as the solvent. After completion, the reaction was concentrated under reduced pressure. To this crude material, 10% NaHCO₃ solution was added, and the mixture was extracted with DCM. The combined organic layer was washed with brine solution, dried over sodium sulfate and filtered, and the filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to afford 5-fluoro-N-isopropyl-2-((5-(6-((6-methyl-5,6,7,8-tetrahydro-1,6-naphthyridin-4-yl)oxy)-2-azaspiro[3.3]heptan-2-yl)-1,2,4-triazin-6-yl)oxy)-N-((2s,4r)-5-methyl-5-azaspiro[3.4]octan-2-yl)benzamide as a solid.

Yield: 17.81%; ¹H NMR (400 MHz, DMSO-d₆): δ 8.46 (s, 1H), 8.23-8.15 (m, 1H), 7.49-7.13 (m, 3H), 6.72-6.60 (m, 1H), 4.86-4.51 (m, 3H), 4.37-3.83 (m, 3H), 3.75-3.56 (m, 2H), 3.37 (br s, 2H), 2.94-2.73 (m, 5H), 2.67-2.59 (m, 3H), 2.40-2.15 (m, 8H), 2.00-1.62 (m, 5H), 1.56-1.39 (m, 3H), 1.28-0.57 (m, 5H); LCMS (Method H): Rt=1.03 min, m/z=657.3 [M+H]⁺; HPLC (Method G): Rt=6.31 min, 98.10%; Chiral SFC (Method K): Rt=3.02 min, 96.80%.

### Example 226. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(7-methyl-7-azaspiro[3.5]nonan-2-yl)benzamide (Compound No. 226)

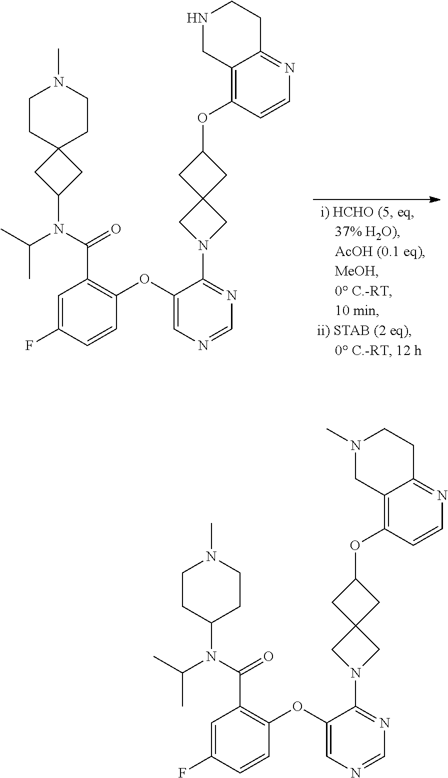 [SMILES: CC(C)N(C(=O)c1cc(F)ccc1Oc1cncnc1N1CC2(CC(Oc3ccnc4c3CNCC4)C2)C1)C1CC2(CCN(C)CC2)C1 ; CC(C)N(C(=O)c1cc(F)ccc1Oc1cncnc1N1CC2(CC(Oc3ccnc4c3CN(C)CC4)C2)C1)C1CCN(C)CC1]

To a stirred solution of 5-fluoro-N-isopropyl-N-(7-methyl-7-azaspiro[3.5]nonan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (350 mg, 0.534 mmol) in MeOH (5 mL), formaldehyde (0.210 mL, 2.67 mmol, 37% in H₂O) and acetic acid (3.06 μL, 0.053 mmol) were added at 0° C. The reaction was stirred at the same temperature for 10 min, then sodium triacetoxyborohydride (226 mg, 1.067 mmol) was added at 0° C. The reaction was stirred at RT for 12 h, and the progress was monitored by TLC (10% MeOH in DCM) and LCMS. After completion, the reaction was concentrated under reduced pressure. The residue was quenched with aqueous NaHCO₃ solution (10 mL), and the mixture was extracted with 10% MeOH in DCM (3×30 mL). The combined organic layer was dried over anhydrous sodium sulfate and filtered, and the filtrate was concentrated under reduced pressure. The residue (520 mg) was purified by reverse phase chromatography using a SiliCycle SiliaSep C18 column (80 g) on Biotage isolera one (Mobile phase A: 10 mM NH₄HCO₃ in water, B: ACN). The pure fractions were concentrated under reduced pressure to obtain 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-(7-methyl-7-azaspiro[3.5]nonan-2-yl)benzamide (110.77 mg, 30.6% yield) as a solid. ¹H NMR (400 MHz, DMSO-d₆): δ 8.31-8.24 (m, 1H), 8.18 (d, J=5.7 Hz, 1H), 7.80-7.67 (m, 1H), 7.31-7.04 (m, 3H), 6.70-6.62 (m, 1H), 4.75 (quin, J=6.5 Hz, 1H), 4.35-4.09 (m, 4H), 4.04-3.85 (m, 1H), 3.80-3.61 (m, 1H), 3.37 (br s, 2H), 2.88-2.74 (m, 5H), 2.65-2.61 (m, 2H), 2.36 (s, 3H), 2.30-2.03 (m, 8H), 1.94-1.56 (m, 6H), 1.53-1.22 (m, 4H), 1.09-0.91 (m, 4H); LCMS (Method H): Rt=1.03 min, m/z=670.3 [M+H]⁺; HPLC (Method C): Rt=8.18 min, 98.43%.

### Example 227. 2-((4-(6-((6-Acetyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide (Compound No. 227)

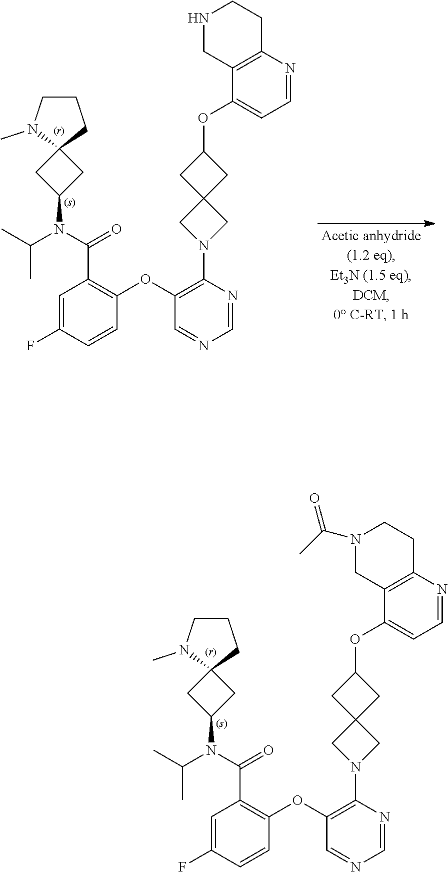 [SMILES: CC(C)N(C(=O)c1cc(F)ccc1Oc1cncnc1N1CC2(CC(Oc3ccnc4c3CNCC4)C2)C1)[C@H]1C[C@@]2(CCCN2C)C1 ; CC(=O)N1CCc2nccc(OC3CC4(C3)CN(c3ncncc3Oc3ccc(F)cc3C(=O)N(C(C)C)[C@H]3C[C@@]5(CCCN5C)C3)C4)c2C1]

In a dried, 25 mL two neck round bottom flask under a nitrogen atmosphere, 5-fluoro-N-isopropyl-N-((2s,4r)-5-methyl-5-azaspiro[3.4]octan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (0.3 g, 0.467 mmol) was dissolved in DCM (2 mL). To this solution, TEA (0.098 mL, 0.701 mmol) and acetic anhydride (0.053 mL, 0.561 mmol) were added at 0° C. The reaction was stirred at RT for 1 h, and the progress was monitored by TLC (10% MeOH in DCM). After completion, the reaction was quenched with aqueous sodium bicarbonate (20 mL) and the mixture was extracted with 10% MeOH in DCM (2×20 mL). The combined organic layer was dried over anhydrous sodium sulfate and filtered, and the filtrate was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN). The product-containing fractions were concentrated under reduced pressure and lyophilized to afford 2-((4-(6-((6-acetyl-5,6,7,8-tetrahydro-1,6-naphthyridin-4-yl)oxy)-2-azaspiro[3.3]heptan-2-yl)pyrimidin-5-yl)oxy)-5-fluoro-N-isopropyl-N-((2s,4r)-5-methyl-5-azaspiro[3.4]octan-2-yl)benzamide (154 mg, 47.7% yield) as a solid. ¹H NMR (400 MHz, DMSO-d₆): δ 8.25-8.25 (m, 1H), 8.30-8.21 (m, 1H), 7.79-7.57 (m, 1H), 7.33-7.06 (m, 3H), 6.78-6.69 (m, 1H), 4.82-4.73 (m, 1H), 4.47 (br s, 2H), 4.39-4.10 (m, 4H), 3.94-3.62 (m, 4H), 2.98-2.69 (m, 6H), 2.47-2.38 (m, 1H), 2.35-2.18 (m, 6H), 2.13-2.06 (m, 3H), 2.05-1.95 (m, 2H), 1.93-1.83 (m, 1H), 1.76-1.67 (m, 1H), 1.58-1.28 (m, 4H), 1.12-0.88 (m, 4H); LCMS (Method C): Rt=1.06 min, m/z=684.4 [M+H]⁺; HPLC (Method G): Rt=6.64 min, 98.97%; Chiral SFC (Method A): Rt=4.47 min, 97.76%.

### Example 228. N-(3-(Dimethylamino)azetidin-1-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (Compound No. 228)

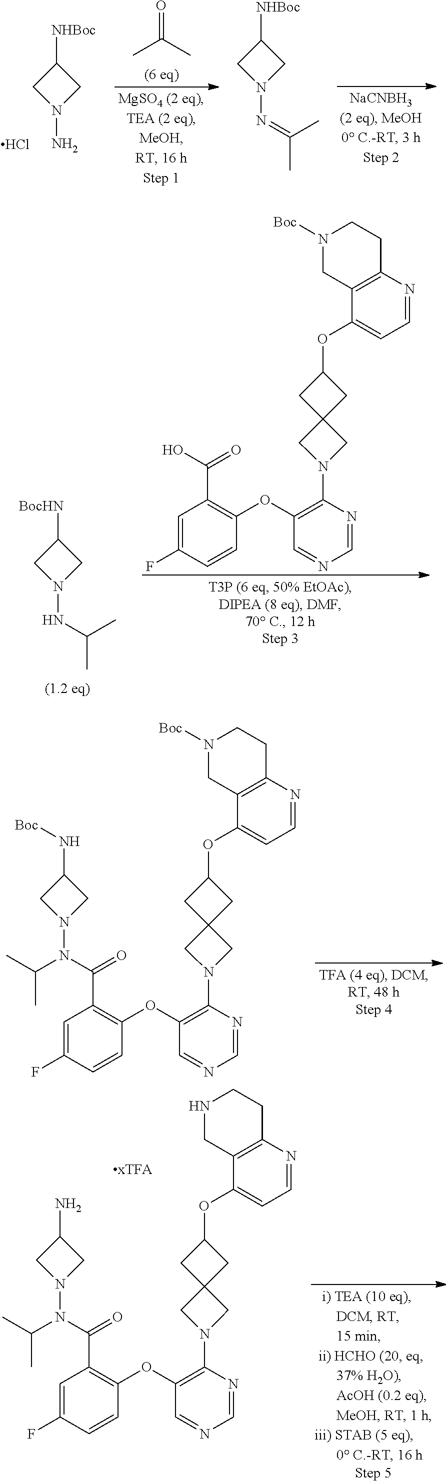

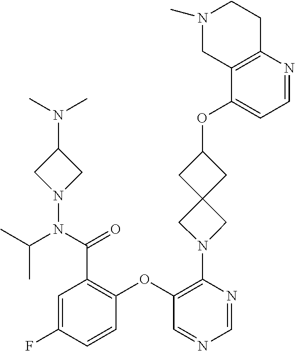 [SMILES: CC(C)N(C(=O)c1cc(F)ccc1Oc1cncnc1N1CC2(CC(Oc3ccnc4c3CN(C)CC4)C2)C1)N1CC(N(C)C)C1]

### Step 1. tert-Butyl (1-(propan-2-ylideneamino)azetidin-3-yl)carbamate

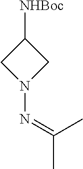 [SMILES: CC(C)=NN1CC(NC(=O)OC(C)(C)C)C1]

To a stirred solution of tert-butyl (1-aminoazetidin-3-yl)carbamate hydrochloride (1.0 g, 4.47 mmol) in MeOH (2 mL), TEA (1.257 mL, 8.94 mmol) was added at RT. The mixture was stirred at RT for 15 min, then was concentrated under reduced pressure. The residue was dissolved in MeOH (2.0 mL), then MgSO₄ (1.076 g, 8.94 mmol) and acetone (1.987 mL, 26.8 mmol) were added at RT. The reaction was stirred at RT for 12 h, and the progress was monitored by LCMS and TLC (10% MeOH in DCM). The reaction was filtered, and the filtrate was concentrated under reduced pressure to afford crude tert-butyl (1-(propan-2-ylideneamino)azetidin-3-yl)carbamate (1.6 g, 48.4% yield). This material was used without further purification. LCMS (Method H): Rt=1.16 min, m/z=228.3 [M+H]⁺, 30.72%.

### Step 2. tert-Butyl (1-(isopropylamino)azetidin-3-yl)carbamate

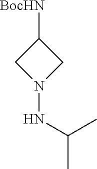 [SMILES: CC(C)NN1CC(NC(=O)OC(C)(C)C)C1]

To a stirred solution of tert-butyl (1-(propan-2-ylideneamino)azetidin-3-yl)carbamate (1.6 g, 7.04 mmol) in MeOH (20 mL), sodium cyanoborohydride (0.885 g, 14.08 mmol) was added at 0° C. The reaction was stirred at RT for 3 h, and the progress was monitored by LCMS and TLC (10% MeOH in DCM). After completion, the reaction was concentrated under reduced pressure. The crude was dissolved in 10% MeOH in DCM (30 mL) and washed with water (30 mL). The organic layer was separated and the aqueous layer was re-extracted with 10% MeOH in DCM (2×20 mL). The combined organic layer was dried over sodium sulfate and filtered, and the filtrate was concentrated under reduced pressure to afford crude tert-butyl (1-(isopropylamino)azetidin-3-yl)carbamate (1.1 g, 61.5% yield) as an oil. This material was used without further purification. LCMS (Method H): Rt=1.28 min, m/z=230.1 [M+H]⁺, 90.25%.

### Step 3. tert-Butyl 4-((2-(5-(2-((3-((ter-butoxycarbonyl)amino)azetidin-1-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate

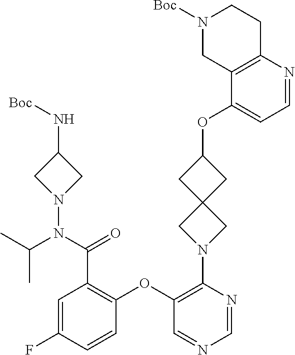 [SMILES: CC(C)N(C(=O)c1cc(F)ccc1Oc1cncnc1N1CC2(CC(Oc3ccnc4c3CN(C(=O)OC(C)(C)C)CC4)C2)C1)N1CC(NC(=O)OC(C)(C)C)C1]

To a mixture of 2-((4-(6-((6-(tert-butoxycarbonyl)-5,6,7,8-tetrahydro-1,6-naphthyridin-4-yl)oxy)-2-azaspiro[3.3]heptan-2-yl)pyrimidin-5-yl)oxy)-5-fluorobenzoic acid (500 mg, 0.866 mmol) and tert-butyl (1-(isopropylamino)azetidin-3-yl)carbamate (238 mg, 1.039 mmol), DIPEA (1.210 mL, 6.93 mmol) and T3P (3.07 mL, 5.19 mmol, 50% in EtOAc) were added at RT. The reaction was stirred at 70° C. for 12 h and monitored by TLC (10% MeOH in DCM) and LCMS. After completion, the reaction was diluted with 10% NaHCO₃ solution (25 mL) and the mixture was extracted with 10% MeOH in DCM (3×30 mL). The combined organic layer was washed with cold brine solution (35 mL), dried over anhydrous sodium sulfate and filtered, and the filtrate was concentrated under reduced pressure. The crude (650 mg) was purified by reverse phase chromatography using a SiliCycle SiliaSep C18 column (80 g cartridge) to obtain tert-butyl 4-((2-(5-(2-((3-((tert-butoxycarbonyl)amino)azetidin-1-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate (180 mg, 25.10% yield) as a solid. ¹H NMR (400 MHz, DMSO-d₆): δ 8.31-8.20 (m, 2H), 7.79-7.64 (m, 1H), 7.40-7.02 (m, 4H), 6.78-6.70 (m, 1H), 4.78 (quin, J=6.4 Hz, 1H), 4.37 (br d, J=2.4 Hz, 2H), 4.32-4.01 (m, 6H), 3.79-3.53 (m, 3H), 3.51-3.41 (m, 2H), 2.85-2.76 (m, 4H), 2.34-2.15 (m, 2H), 1.43 (s, 9H), 1.40-1.32 (m, 12H), 1.06-0.90 (m, 3H), one proton merged with solvent peaks; LCMS (Method A): Rt=2.35 min, m/z=789.3 [M+H]⁺; HPLC (Method C): Rt=11.47 min, 89.21%.

### Step 4. N-(3-Aminoazetidin-1-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xTFA

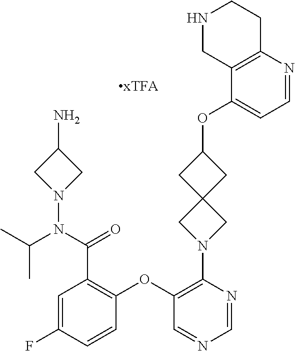 [SMILES: CC(C)N(C(=O)c1cc(F)ccc1Oc1cncnc1N1CC2(CC(Oc3ccnc4c3CNCC4)C2)C1)N1CC(N)C1]

To a stirred solution of tert-butyl 4-((2-(5-(2-((3-((tert-butoxycarbonyl)amino)azetidin-1-yl)(isopropyl)carbamoyl)-4-fluorophenoxy)pyrimidin-4-yl)-2-azaspiro[3.3]heptan-6-yl)oxy)-7,8-dihydro-1,6-naphthyridine-6(5H)-carboxylate (140 mg, 0.177 mmol) in DCM (1.5 mL), trifluoroacetic acid (0.055 mL, 0.710 mmol) was added at 0° C. The reaction was continued at RT for 48 h, and was monitored by LCMS. When complete, the reaction was concentrated under reduced pressure. The residue was reconcentrated from DCM (3×20 mL) to obtain crude N-(3-aminoazetidin-1-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xTFA (150 mg, 61.8% yield). This material was used without further purification. LCMS (Method H): Rt=0.96 min, m/z=589.3 [M+H]⁺; 23.01%.

### Step 5. N-(3-(Dimethylamino)azetidin-1-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

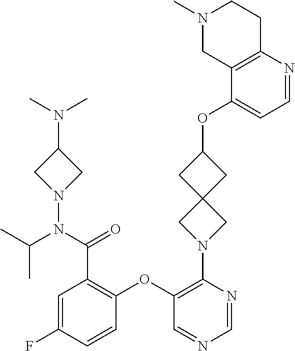 [SMILES: CC(C)N(C(=O)c1cc(F)ccc1Oc1cncnc1N1CC2(CC(Oc3ccnc4c3CN(C)CC4)C2)C1)N1CC(N(C)C)C1]

To a stirred solution of N-(3-aminoazetidin-1-yl)-5-fluoro-N-isopropyl-2-((4-(6-((5,6,7,8-tetrahydro-1,6-naphthyridin-4-yl)oxy)-2-azaspiro[3.3]heptan-2-yl)pyrimidin-5-yl)oxy)benzamide xTFA (150 mg, 0.184 mmol) in DCM (3 mL), triethylamine (0.256 mL, 1.837 mmol) was added at RT. The reaction was stirred at RT for 15 min, then was concentrated under reduced pressure, and the residue was dissolved in MeOH (3 mL). To this mixture, acetic acid (3.15 μL, 0.055 mmol) and formaldehyde (0.289 mL, 3.67 mmol) were added at RT. The reaction was stirred at RT for 1 h, then sodium triacetoxyborohydride (195 mg, 0.918 mmol) was added at 0° C. The reaction was stirred at RT for 16 h, and the progress was monitored by LCMS. After completion, the reaction was concentrated under reduced pressure. The residue was dissolved in 10% MeOH in DCM (20 mL) and the solution was washed with aqueous sodium bicarbonate (2×10 mL). The combined organic layer was dried over sodium sulfate and filtered, and the filtrate was concentrated under reduced pressure. The crude was purified by reverse phase chromatography using a SiliCycle SiliaSep C18 column (120 g) on Biotage isolera one (Mobile phase A: 10 mM NH₄HCO₃ in water, B: ACN). The pure fractions were concentrated under reduced pressure to afford N-(3-(dimethylamino)azetidin-1-yl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (6.5 mg, 5.38% yield) as a solid. ¹H NMR (400 MHz, DMSO-d₆): δ 8.33-8.22 (m, 1H), 8.18 (d, J=5.5 Hz, 1H), 7.79-7.61 (m, 1H), 7.37-7.23 (m, 1H), 7.22-7.01 (m, 2H), 6.71-6.60 (m, 1H), 4.75 (quin, J=6.4 Hz, 1H), 4.32-4.08 (m, 5H), 3.96-3.87 (m, 1H), 3.63-3.50 (m, 1H), 3.44-3.38 (m, 1H), 3.36 (br s, 2H), 3.05-2.96 (m, 1H), 2.86-2.75 (m, 4H), 2.63 (br t, J=5.7 Hz, 2H), 2.36 (s, 3H), 2.30-2.22 (m, 2H), 2.08 (s, 3H), 1.93 (s, 3H), 1.45-0.89 (m, 6H), one proton merged with solvent peaks; LCMS (Method H): Rt=0.98 min, m/z=631.3 [M+H]⁺; HPLC (Method G): Rt=6.17 min, 95.90%.

### Example 229. 1-Acetoxyethyl 4-((2-(5-(4-fluoro-2-(isopropyl((2s,4r)-5-methyl-5-azaspiro[3.4]octan-2-yl)carbamoyl)phenoxy)pyrimidin-4-yl)-2-azaspiro[3.3]heptan-6-yl)oxy)-7,8-dihydro-1,6-naphthyridine-6(5H)-carboxylate (Compound No. 229)

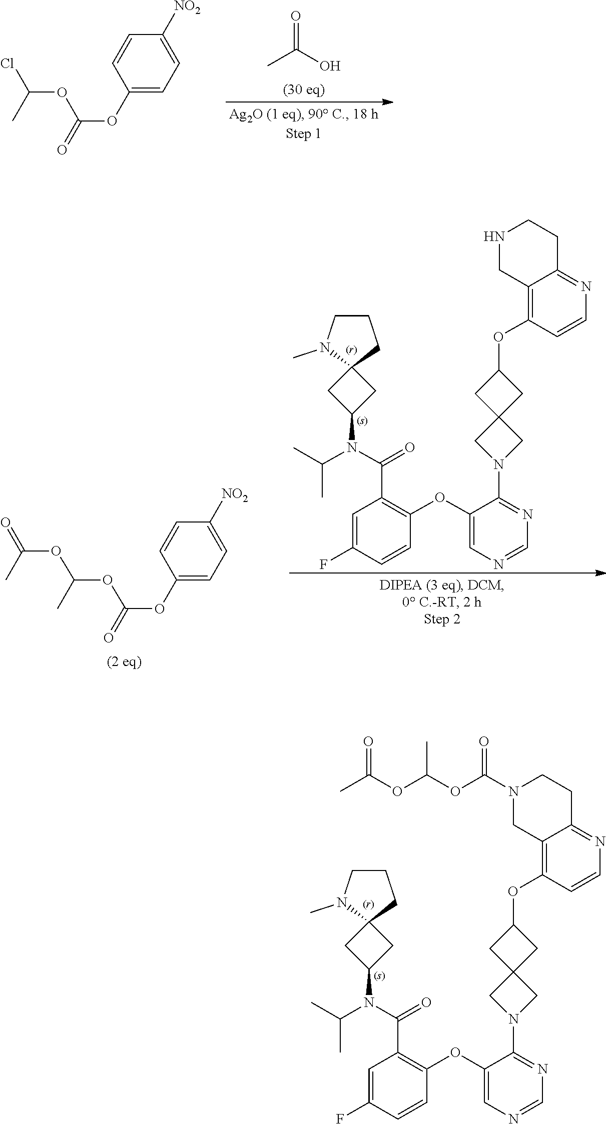

### Step 1. 1-(((4-Nitrophenoxy)carbonyl)oxy)ethyl acetate

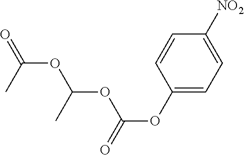 [SMILES: CC(=O)OC(C)OC(=O)Oc1ccc([N+](=O)[O-])cc1]

To a stirred solution of 1-chloroethyl (4-nitrophenyl) carbonate (1 g, 4.07 mmol) in AcOH (6.99 mL, 122 mmol), silver oxide (0.943 g, 4.07 mmol) was added at RT. The reaction was stirred at 90° C. for 18 h. The reaction was monitored by TLC (30% EtOAc in hexane). After completion, the reaction was diluted with water (25 mL) and the mixture was extracted with EtOAc (2×25 mL). The combined organic layer was dried over anhydrous sodium sulfate and filtered, and the filtrate was concentrated under reduced pressure to afford crude 1-(((4-nitrophenoxy)carbonyl)oxy)ethyl acetate (920 mg, 84% yield) as a liquid. This material was used without further purification.

### Step 2. 1-Acetoxyethyl 4-((2-(5-(4-fluoro-2-(isopropyl((2s,4r)-5-methyl-5-azaspiro[3.4]octan-2-yl)carbamoyl)phenoxy)pyrimidin-4-yl)-2-azaspiro[3.3]heptan-6-yl)oxy)-7,8-dihydro-1,6-naphthyridine-6(5H)-carboxylate

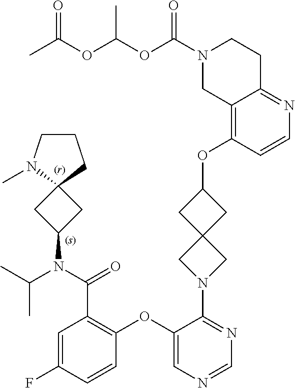 [SMILES: CC(=O)OC(C)OC(=O)N1CCc2nccc(OC3CC4(C3)CN(c3ncncc3Oc3ccc(F)cc3C(=O)N(C(C)C)[C@H]3C[C@@]5(CCCN5C)C3)C4)c2C1]

To a stirred solution of 5-fluoro-N-isopropyl-N-((2s,4r)-5-methyl-5-azaspiro[3.4]octan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (250 mg, 0.390 mmol) and 1-(((4-nitrophenoxy)carbonyl)oxy)ethyl acetate (210 mg, 0.779 mmol) in DCM (5 mL) under a nitrogen atmosphere, DIPEA (0.204 mL, 1.169 mmol) was added at 0° C. The reaction was stirred at RT, and progress was monitored by TLC (10% MeOH in DCM). After 2 h, the reaction was concentrated under reduced pressure. The crude was purified by reverse phase chromatography using a SiliCycle SiliaSep C18 column (40 g) on Biotage isolera one (Mobile phase A: 0.1% formic acid in water, B: ACN). The pure fractions were concentrated under reduced pressure, the residue was diluted with saturated sodium bicarbonate solution (5 mL), and the mixture was extracted with 10% MeOH in DCM (2×10 mL). The combined organic layer was dried over anhydrous sodium sulfate and filtered, and the filtrate was concentrated under reduced pressure and lyophilized to obtain 1-acetoxyethyl 4-((2-(5-(4-fluoro-2-(isopropyl((2s,4r)-5-methyl-5-azaspiro[3.4]octan-2-yl)carbamoyl)phenoxy)pyrimidin-4-yl)-2-azaspiro[3.3]heptan-6-yl)oxy)-7,8-dihydro-1,6-naphthyridine-6(5H)-carboxylate (141 mg, 46.5% yield). ¹H NMR (400 MHz, DMSO-d₆): δ 8.31-8.22 (m, 2H), 7.79-7.57 (m, 1H), 7.33-7.06 (m, 3H), 6.79-6.64 (m, 2H), 4.79 (quin, J=6.6 Hz, 1H), 4.42 (br s, 2H), 4.37-4.12 (m, 4H), 3.95-3.63 (m, 4H), 3.01-2.69 (m, 6H), 2.43-2.19 (m, 7H), 2.03 (s, 3H), 2.01-1.86 (m, 3H), 1.77-1.67 (m, 1H), 1.59-1.48 (m, 2H), 1.45 (d, J=5.4 Hz, 3H), 1.35-0.89 (m, 5H), one proton merged with solvent peaks; LCMS (Method H): Rt=1.24 min; m/z=772.3 [M+H]⁺; HPLC (Method H): Rt=6.29 min, 99.17%;

### Example 230. 1-((L-Valyl)oxy)ethyl 4-((2-(5-(4-fluoro-2-(isopropyl((2s,4r)-5-methyl-5-azaspiro[3.4]octan-2-yl)carbamoyl)phenoxy)pyrimidin-4-yl)-2-azaspiro[3.3]heptan-6-yl)oxy)-7,8-dihydro-1,6-naphthyridine-6(5H)-carboxylate dihydrochloride (Compound No. 230)

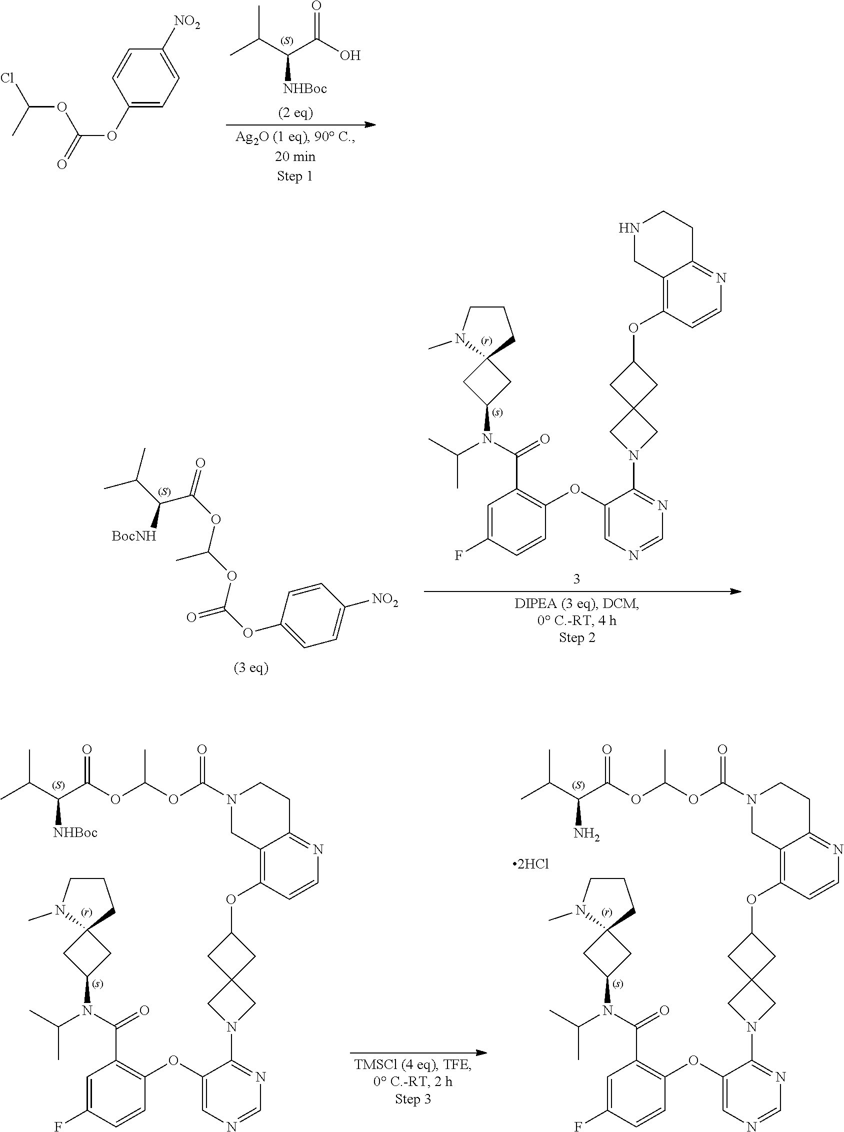

### Step 1. 1-(((4-Nitrophenoxy)carbonyl)oxy)ethyl(tert-butoxycarbonyl)-L-valinate

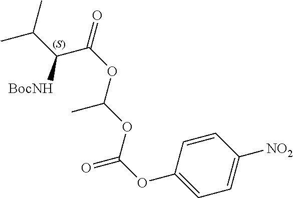

A mixture of 1-chloroethyl (4-nitrophenyl) carbonate (3 g, 12.21 mmol), (tert-butoxycarbonyl)-L-valine (5.31 g, 24.43 mmol), and silver oxide (2.83 g, 12.21 mmol) was heated at 90° C. for 20 min, and the reaction was monitored by TLC (30% EtOAc in hexane). After completion, the reaction was filtered through a Celite® pad, and the pad was washed with EtOAc (100 mL). The filtrate was washed with saturated sodium bicarbonate solution (50 mL), dried over anhydrous sodium sulfate and filtered, and the filtrate was concentrated under reduced pressure. The crude product was purified by silica gel flash column chromatography using EtOAc in hexane (product eluted at 30% EtOAc in hexane) to obtain impure 1-(((4-nitrophenoxy)carbonyl)oxy)ethyl (tert-butoxycarbonyl)-L-valinate (1.5 g, 28.8% yield) as a sticky liquid. This material was used without further purification.

### Step 2. 1-(((tert-Butoxycarbonyl)-L-valyl)oxy)ethyl 4-((2-(5-(4-fluoro-2-(isopropyl((2s,4r)-5-methyl-5-azaspiro[3.4]octan-2-yl)carbamoyl)phenoxy)pyrimidin-4-yl)-2-azaspiro[3.3]heptan-6-yl)oxy)-7,8-dihydro-1,6-naphthyridine-6(5H)-carboxylate

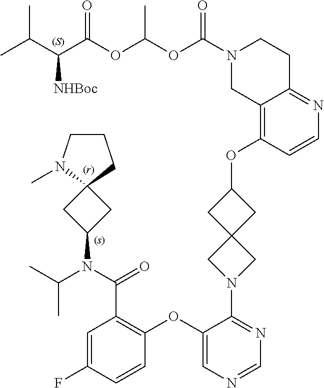

To a stirred solution of 5-fluoro-N-isopropyl-N-((2s,4r)-5-methyl-5-azaspiro[3.4]octan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide (400 mg, 0.623 mmol) and 1-(((4-nitrophenoxy)carbonyl)oxy)ethyl (tert-butoxycarbonyl)-L-valinate (532 mg, 1.247 mmol) in DCM (8 mL), DIPEA (0.327 mL, 1.870 mmol) was added at 0° C. The reaction was stirred at RT for 2 h under a nitrogen atmosphere. The starting material was not consumed. Therefore, another portion of 1-(((4-nitrophenoxy)carbonyl)oxy)ethyl (tert-butoxycarbonyl)-L-valinate (532 mg, 1.247 mmol) was added at 0° C., and the reaction was stirred at RT for another 2 h. The reaction progress was monitored by TLC (10% MeOH in DCM). After completion, the reaction was concentrated under reduced pressure. The crude was purified by reverse phase chromatography using a SiliCycle SiliaSep C18 column (40 g) on Biotage isolera one (Mobile phase A: 0.1% formic acid in water, B: ACN). The pure fractions were concentrated under reduced pressure and lyophilized to obtain impure product (500 mg). The isolated product showed presence of 4-nitrophenol impurity by LCMS and TLC. Therefore, the material was dissolved in DCM (20 mL) and the solution was washed with 10% NaOH (2×10 mL). The combined organic layer was dried over sodium sulfate and filtered, and the filtrate was concentrated under reduced pressure to obtain 1-(((tert-butoxycarbonyl)-L-valyl)oxy)ethyl 4-((2-(5-(4-fluoro-2-(isopropyl((2s,4r)-5-methyl-5-azaspiro[3.4]octan-2-yl)carbamoyl)phenoxy)pyrimidin-4-yl)-2-azaspiro[3.3]heptan-6-yl)oxy)-7,8-dihydro-1,6-naphthyridine-6(5H)-carboxylate (210 mg, 33.9% yield) as a solid. ¹H NMR (400 MHz, DMSO-d₆): δ 8.29-8.20 (m, 2H), 7.80-7.56 (m, 1H), 7.33-7.05 (m, 4H), 6.79-6.68 (m, 2H), 4.84-4.71 (m, 1H), 4.49-4.09 (m, 6H), 3.94-3.57 (m, 6H), 2.88-2.75 (m, 5H), 2.34-2.21 (m, 5H), 2.08-1.86 (m, 4H), 1.76-1.68 (m, 1H), 1.59-1.43 (m, 7H), 1.41-1.27 (m, 11H), 1.09-0.82 (m, 10H); LCMS (Method B): Rt=1.45 min; m/z=929.4 [M+H]⁺, 93.42%.

### Step 3. 1-((L-Valyl)oxy)ethyl 4-((2-(5-(4-fluoro-2-(isopropyl((2s,4r)-5-methyl-5-azaspiro[3.4]octan-2-yl)carbamoyl)phenoxy)pyrimidin-4-yl)-2-azaspiro[3.3]heptan-6-yl)oxy)-7,8-dihydro-1,6-naphthyridine-6(5H)-carboxylate Dihydrochloride

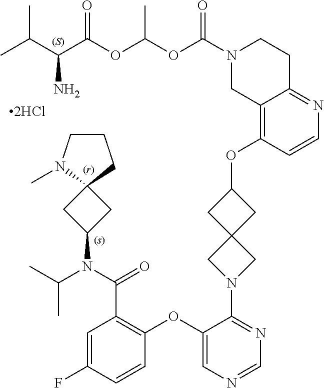

To a stirred solution of 1-(((tert-butoxycarbonyl)-L-valyl)oxy)ethyl 4-((2-(5-(4-fluoro-2-(isopropyl((2s,4r)-5-methyl-5-azaspiro[3.4]octan-2-yl)carbamoyl)phenoxy)pyrimidin-4-yl)-2-azaspiro[3.3]heptan-6-yl)oxy)-7,8-dihydro-1,6-naphthyridine-6(5H)-carboxylate (180 mg, 0.194 mmol) in TFE (5 mL), TMSCl (0.099 mL, 0.775 mmol) was added dropwise at −20° C. The reaction was stirred at RT for 2 h. The reaction progress was monitored by TLC (10% MeOH in DCM) and LCMS. After completion, the reaction was concentrated under reduced pressure. The residue was reconcentrated from ACN (4×2 mL) and lyophilized to obtain 1-((L-valyl)oxy)ethyl 4-((2-(5-(4-fluoro-2-(isopropyl((2s,4r)-5-methyl-5-azaspiro[3.4]octan-2-yl)carbamoyl)phenoxy)pyrimidin-4-yl)-2-azaspiro[3.3]heptan-6-yl)oxy)-7,8-dihydro-1,6-naphthyridine-6(5H)-carboxylate dihydrochloride (148 mg, 83% yield) as a solid. ¹H NMR (400 MHz, DMSO-d₆): δ 11.53-11.04 (m, 1H), 8.67 (br d, J=5.1 Hz, 1H), 8.52 (br d, J=16.6 Hz, 4H), 7.90 (br s, 1H), 7.43-7.23 (m, 4H), 6.89-6.79 (m, 1H), 5.18-5.03 (m, 1H), 4.61-4.25 (m, 7H), 4.13 (br s, 1H), 3.95 (br s, 1H), 3.85-3.64 (m, 5H), 3.14-2.87 (m, 7H), 2.82 (br d, J=4.5 Hz, 3H), 2.74-2.63 (m, 1H), 2.25-2.06 (m, 3H), 1.96-1.78 (m, 2H), 1.54 (br d, J=4.5 Hz, 3H), 1.21-1.04 (m, 3H), 1.01-0.90 (m, 9H), one proton merged with solvent peaks; LCMS (Method B): Rt=0.95 min, m/z=829.4 [M+H]⁺; HPLC (Method H): Rt=5.74 min, 98.08%; Chiral SFC (Method AAQ): Rt=1.14 min, 100%.

### Example 231. 1-(Pivaloyloxy)ethyl 4-((2-(5-(4-fluoro-2-(isopropyl((2s,4r)-5-methyl-5-azaspiro[3.4]octan-2-yl)carbamoyl)phenoxy)pyrimidin-4-yl)-2-azaspiro[3.3]heptan-6-yl)oxy)-7,8-dihydro-1,6-naphthyridine-6(5H)-carboxylate (Compound No. 231)

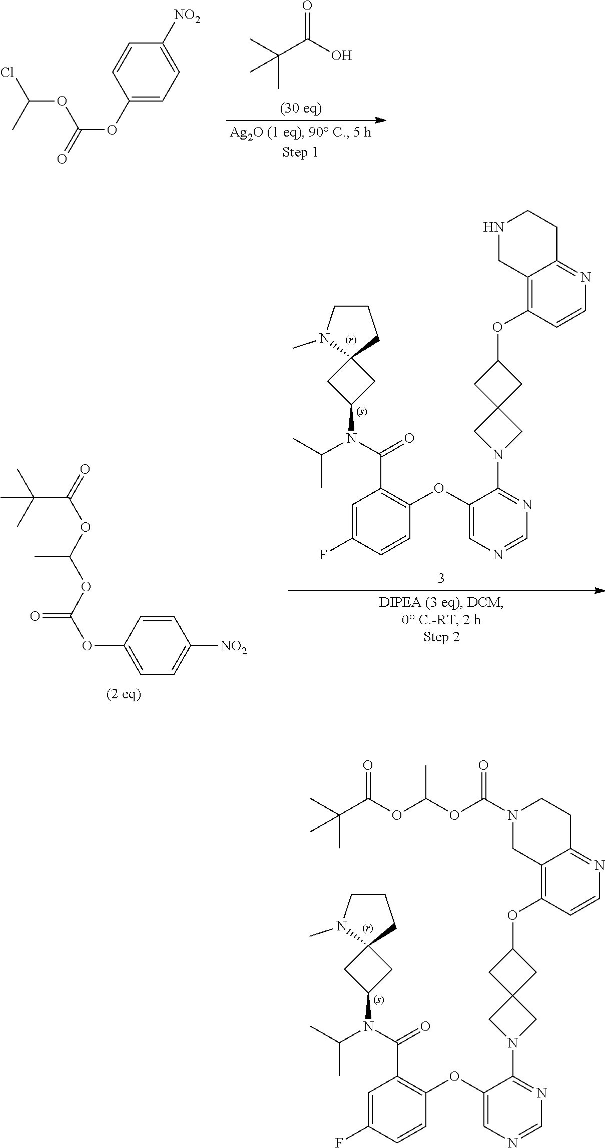

### Step 1. 1-(((4-Nitrophenoxy)carbonyl)oxy)ethyl Pivalate

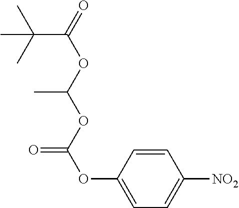 [SMILES: CC(OC(=O)Oc1ccc([N+](=O)[O-])cc1)OC(=O)C(C)(C)C]

A mixture of 1-chloroethyl (4-nitrophenyl) carbonate (1 g, 4.07 mmol), pivalic acid (12.47 g, 122 mmol), and silver oxide (0.943 g, 4.07 mmol) was heated at 90° C. for 5 h, and the reaction was monitored by TLC (30% EtOAc in hexane). The reaction was quenched with water (25 mL) and extracted with EtOAc (2×25 mL). The combined organic layer was dried over anhydrous sodium sulfate and filtered, and the filtrate was concentrated under reduced pressure crude 1-(((4-nitrophenoxy)carbonyl)oxy)ethyl pivalate (940 mg, 74.2% yield) as a liquid. This material was used without further purification. ¹H NMR (400 MHz, DMSO-d₆). δ 8.37-8.32 (m, 2H), 7.60-7.54 (m, 2H), 6.73 (q, J=5.4 Hz, 1H), 1.56 (d, J=5.4 Hz, 3H), 1.17 (s, 9H).

### Step 2. 1-(Pivaloyloxy)ethyl 4-((2-(5-(4-fluoro-2-(isopropyl((2s,4r)-5-methyl-5-azaspiro[3.4]octan-2-yl)carbamoyl)phenoxy)pyrimidin-4-yl)-2-azaspiro[3.3]heptan-6-yl)oxy)-7,8-dihydro-1,6-naphthyridine-6(5H)-carboxylate

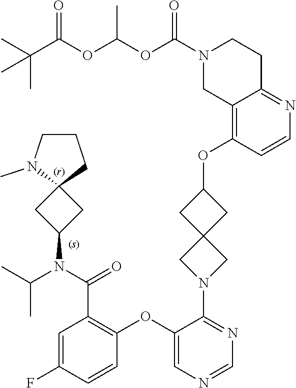

This compound was synthesized following the procedure described for the synthesis of Example 229, step 2, starting with 1 equivalent of 5-fluoro-N-isopropyl-N-(5-methyl-5-azaspiro[3.4]octan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide and 2 equivalents of 1-(((4-nitrophenoxy)carbonyl)oxy)ethyl pivalate. After completion, the reaction was concentrated under reduced pressure. The crude was purified by reverse phase chromatography using a SiliCycle SiliaSep C18 column (40 g) on Biotage isolera one (Mobile phase A: 10 mM ABC in water, B: ACN). The pure fractions were concentrated under reduced pressure and lyophilized to afford 1-(pivaloyloxy)ethyl 4-((2-(5-(4-fluoro-2-(isopropyl(5-methyl-5-azaspiro[3.4]octan-2-yl)carbamoyl)phenoxy)pyrimidin-4-yl)-2-azaspiro[3.3]heptan-6-yl)oxy)-7,8-dihydro-1,6-naphthyridine-6(5H)-carboxylate as a solid.

Yield: 36.7%; ¹H NMR (400 MHz, DMSO-d₆): δ 8.31-8.21 (m, 2H), 7.81-7.59 (m, 1H), 7.33-7.06 (m, 3H), 6.77-6.72 (m, 1H), 6.70-6.59 (m, 11H), 4.78 (quin, J=6.3 Hz, 1H), 4.41 (br s, 2H), 4.35-4.08 (m, 4H), 3.95-3.58 (m, 4H), 3.00-2.90 (m, 1H), 2.87-2.71 (m, 5H), 2.44-2.37 (m, 2H), 2.30-2.18 (m, 3H), 2.08-1.85 (m, 4H), 1.76-1.68 (m, 1H), 1.57-1.48 (m, 2H), 1.46 (d, J=5.4 Hz, 3H), 1.34-0.91 (m, 15H), one proton merged with solvent peaks; LCMS (Method B): Rt=1.25 min, m/z=814.4 [M+H]⁺; HPLC (Method G): Rt=8.08 min, 97.27%.

### Example 232. 1-Acetoxy-2-methylpropyl 4-((2-(5-(4-fluoro-2-(isopropyl((2s,4r)-5-methyl-5-azaspiro[3.4]octan-2-yl)carbamoyl)phenoxy)pyrimidin-4-yl)-2-azaspiro[3.3]heptan-6-yl)oxy)-7,8-dihydro-1,6-naphthyridine-6(5H)-carboxylate (Compound No. 232)

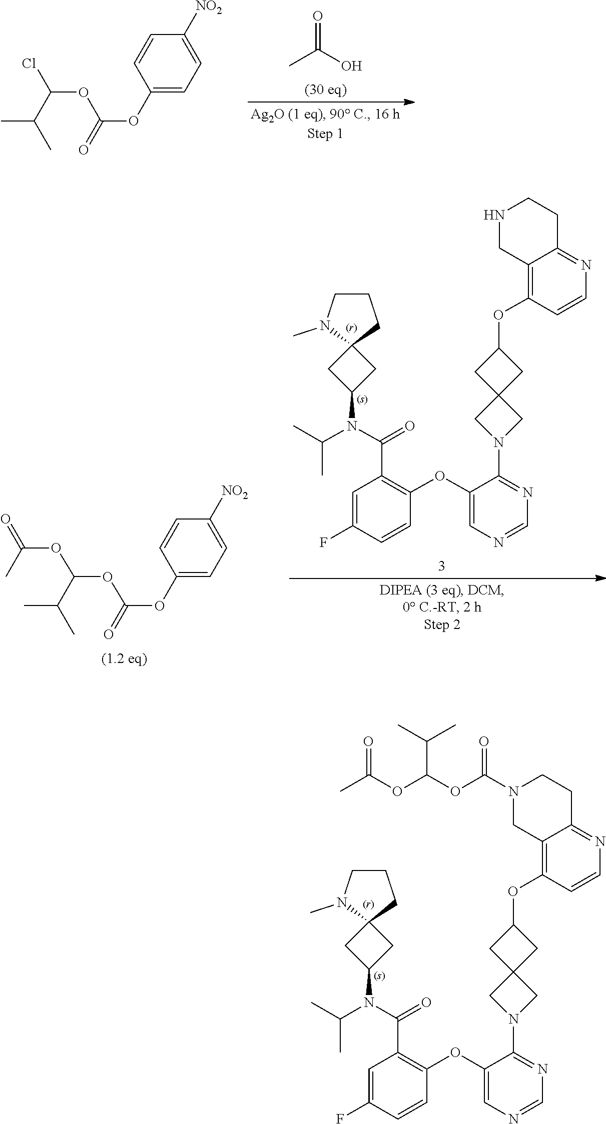

### Step 1. 2-Methyl-1-(((4-nitrophenoxy)carbonyl)oxy)propyl Acetate

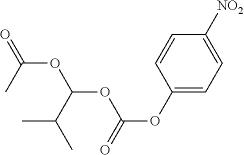 [SMILES: CC(=O)OC(OC(=O)Oc1ccc([N+](=O)[O-])cc1)C(C)C]

To a stirred solution of 1-chloro-2-methylpropyl (4-nitrophenyl) carbonate (1 g, 3.65 mmol) in acetic acid (6.28 mL, 110 mmol), silver oxide (0.847 g, 3.65 mmol) was added at RT. The reaction was stirred at 90° C. and monitored by TLC (30% EtOAc in hexane). After 16 h, the reaction was quenched with water (25 mL) and extracted with EtOAc (2×25 mL). The combined organic layer was dried over anhydrous sodium sulfate and filtered, and the filtrate was concentrated under reduced pressure to afford crude 2-methyl-1-(((4-nitrophenoxy)carbonyl)oxy)propyl acetate (550 mg, 50.6% yield) as a liquid. This material was used without further purification.

### Step 2. 1-Acetoxy-2-methylpropyl 4-((2-(5-(4-fluoro-2-(isopropyl((2s,4r)-5-methyl-5-azaspiro[3.4]octan-2-yl)carbamoyl)phenoxy)pyrimidin-4-yl)-2-azaspiro[3.3]heptan-6-yl)oxy)-7,8-dihydro-1,6-naphthyridine-6(5H)-carboxylate

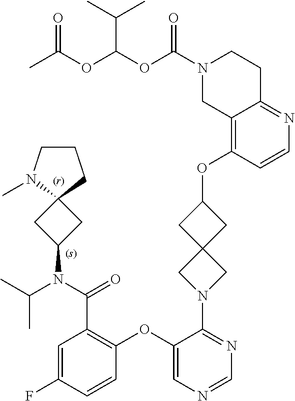 [SMILES: CC(=O)OC(OC(=O)N1CCc2nccc(OC3CC4(C3)CN(c3ncncc3Oc3ccc(F)cc3C(=O)N(C(C)C)[C@H]3C[C@@]5(CCCN5C)C3)C4)c2C1)C(C)C]

This compound was synthesized following the procedure described for the synthesis of Example 229, step 2, starting with 1 equivalent of 5-fluoro-N-isopropyl-N-((2s,4r)-5-methyl-5-azaspiro[3.4]octan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide and 1.2 equivalents of 2-methyl-1-(((4-nitrophenoxy)carbonyl)oxy)propyl acetate. After completion, the reaction was concentrated under reduced pressure. The crude was purified by reverse phase chromatography using a SiliCycle SiliaSep C18 column (40 g) on Biotage isolera one (Mobile phase A: 0.1% FA in water, B: ACN). The pure fractions were concentrated under reduced pressure. The residue was diluted with saturated sodium bicarbonate solution and the mixture was extracted with 10% MeOH in DCM. The combined organic layer was dried over anhydrous sodium sulfate and filtered, and the filtrate was concentrated under reduced pressure and lyophilized to obtain 1-acetoxy-2-methylpropyl 4-((2-(5-(4-fluoro-2-(isopropyl((2s,4r)-5-methyl-5-azaspiro[3.4]octan-2-yl)carbamoyl)phenoxy)pyrimidin-4-yl)-2-azaspiro[3.3]heptan-6-yl)oxy)-7,8-dihydro-1,6-naphthyridine-6(5H)-carboxylate as a solid.

Yield: 55.7%; ¹H NMR (400 MHz, DMSO-d₆): δ 8.30-8.22 (m, 2H), 7.78-7.57 (m, 1H), 7.33-7.06 (m, 3H), 6.79-6.70 (m, 1H), 6.54-6.44 (m, 1H), 4.88-4.73 (m, 1H), 4.50-4.40 (m, 2H), 4.37-4.12 (m, 4H), 3.95-3.62 (m, 4H), 3.00-2.76 (m, 5H), 2.46-2.38 (m, 2H), 2.36-2.20 (m, 5H), 2.09-1.95 (m, 6H), 1.94-1.70 (m, 2H), 1.58-1.26 (m, 5H), 1.11-0.87 (m, 10H); LCMS (Method B): Rt=1.21 min; m/z=800.3 [M+H]⁺; HPLC (Method G): Rt=7.71 min, 99.18%.

### Example 233. 2-Methyl-1-(pivaloyloxy)propyl 4-((2-(5-(4-fluoro-2-(isopropyl((2s,4r)-5-methyl-5-azaspiro[3.4]octan-2-yl)carbamoyl)phenoxy)pyrimidin-4-yl)-2-azaspiro[3.3]heptan-6-yl)oxy)-7,8-dihydro-1,6-naphthyridine-6(5H)-carboxylate (Compound No. 233)

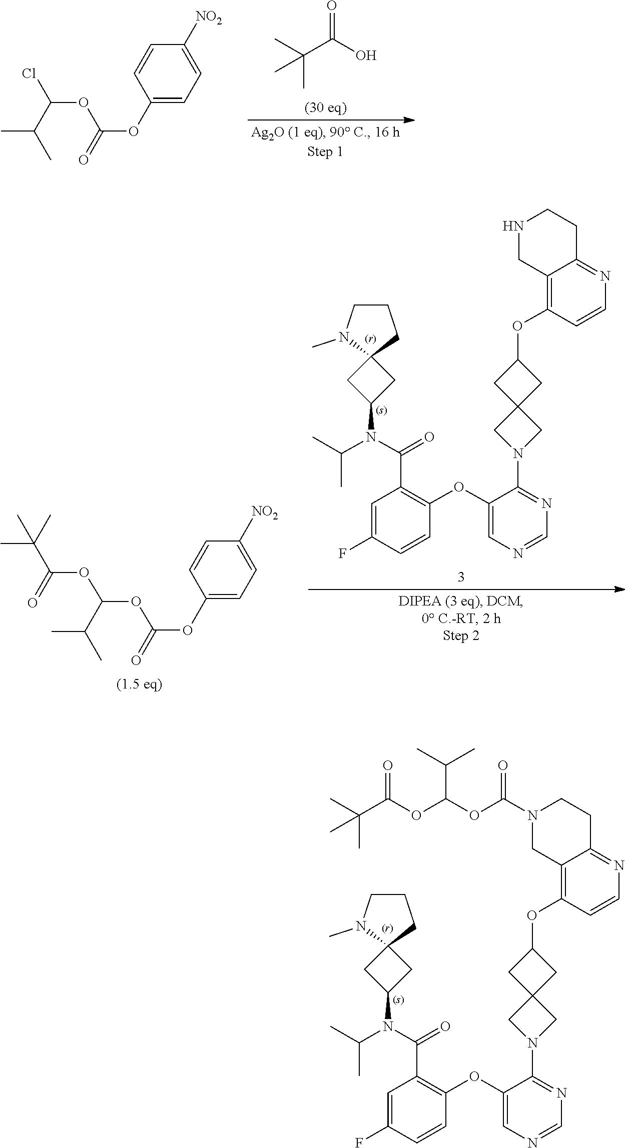

### Step 1. 2-Methyl-1-(((4-nitrophenoxy)carbonyl)oxy)propyl Pivalate

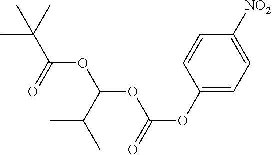 [SMILES: CC(C)C(OC(=O)Oc1ccc([N+](=O)[O-])cc1)OC(=O)C(C)(C)C]

A mixture of 1-chloro-2-methylpropyl (4-nitrophenyl) carbonate (1 g, 3.65 mmol), pivalic acid (11.20 g, 110 mmol), and silver oxide (0.847 g, 3.65 mmol) was heated at 90° C. for 16 h, and the reaction was monitored by TLC (30% EtOAc in hexane). The reaction was quenched with saturated sodium bicarbonate solution (20 mL) and extracted with DCM (2×30 mL). The combined organic layer was washed with 10% NaOH solution (2×20 mL), dried over anhydrous sodium sulfate and filtered, and the filtrate was concentrated under reduced pressure to afford crude 2-methyl-1-(((4-nitrophenoxy)carbonyl)oxy)propyl pivalate (640 mg, 51.6% yield) as gummy liquid. This material was used without further purification.

### Step 2. 2-Methyl-1-(pivaloyloxy)propyl 4-((2-(5-(4-fluoro-2-(isopropyl((2s,4r)-5-methyl-5-azaspiro[3.4]octan-2-yl)carbamoyl)phenoxy)pyrimidin-4-yl)-2-azaspiro[3.3]heptan-6-yl)oxy)-7,8-dihydro-1,6-naphthyridine-6(5H)-carboxylate

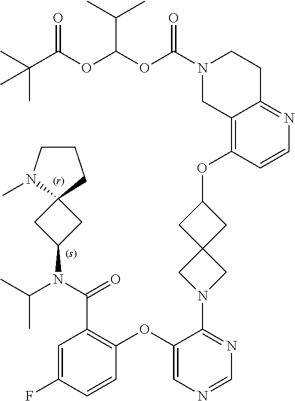 [SMILES: CC(C)C(OC(=O)N1CCc2nccc(OC3CC4(C3)CN(c3ncncc3Oc3ccc(F)cc3C(=O)N(C(C)C)[C@H]3C[C@@]5(CCCN5C)C3)C4)c2C1)OC(=O)C(C)(C)C]

This compound was synthesized following the procedure described for the synthesis of Example 229, step 2, starting with 1 equivalent of 5-fluoro-N-isopropyl-N-((2s,4r)-5-methyl-5-azaspiro[3.4]octan-2-yl)-2-((4-(6-((5,6,7,8-tetrahydro-1,6-naphthyridin-4-yl)oxy)-2-azaspiro[3.3]heptan-2-yl)pyrimidin-5-yl)oxy)benzamide and 1.5 equivalents of 2-methyl-1-(((4-nitrophenoxy)carbonyl)oxy)propyl pivalate. After completion, the reaction was concentrated under reduced pressure. The crude was diluted with saturated sodium bicarbonate solution and the mixture was extracted with DCM. The combined organic layer was washed with 10% NaOH solution, dried over anhydrous sodium sulfate and filtered, and the filtrate was concentrated under reduced pressure. The crude was purified by reverse phase chromatography using a SiliCycle SiliaSep C18 column (40 g) on Biotage isolera one (Mobile phase A: 0.1% formic acid in water, B: ACN). The pure fractions were concentrated under reduced pressure. The residue was diluted with saturated sodium bicarbonate solution and the mixture was extracted with 10% MeOH in DCM. The combined organic layer was dried over anhydrous sodium sulfate and filtered, and the filtrate was concentrated under reduced pressure and lyophilized to obtain 2-methyl-1-(pivaloyloxy)propyl 4-((2-(5-(4-fluoro-2-(isopropyl((2s,4r)-5-methyl-5-azaspiro[3.4]octan-2-yl)carbamoyl)phenoxy)pyrimidin-4-yl)-2-azaspiro[3.3]heptan-6-yl)oxy)-7,8-dihydro-1,6-naphthyridine-6(5H)-carboxylate as a solid.

Yield: 48.96%; ¹H NMR (400 MHz, DMSO-d₆): δ 8.30-8.21 (m, 2H), 7.78-7.53 (m, 1H), 7.35-7.06 (m, 3H), 6.79-6.71 (m, 1H), 6.50-6.36 (m, 1H), 4.83-4.75 (m, 1H), 4.43 (br s, 2H), 4.36-4.07 (m, 4H), 3.94-3.58 (m, 4H), 2.98-2.76 (m, 5H), 2.46-2.37 (m, 2H), 2.35-2.18 (m, 5H), 2.09-1.94 (m, 4H), 1.92-1.85 (m, 1H), 1.75-1.70 (m, 1H), 1.58-1.28 (m, 4H), 1.16-1.04 (m, 11H), 1.01-0.86 (m, 8H); LCMS (Method H): Rt=1.49 min; m/z=842.3 [M+H]⁺; HPLC (Method G): Rt=8.52 min, 97.36%.

### Example 234. 5-Fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-azaspiro[3.5]nonan-2-yl)benzamide

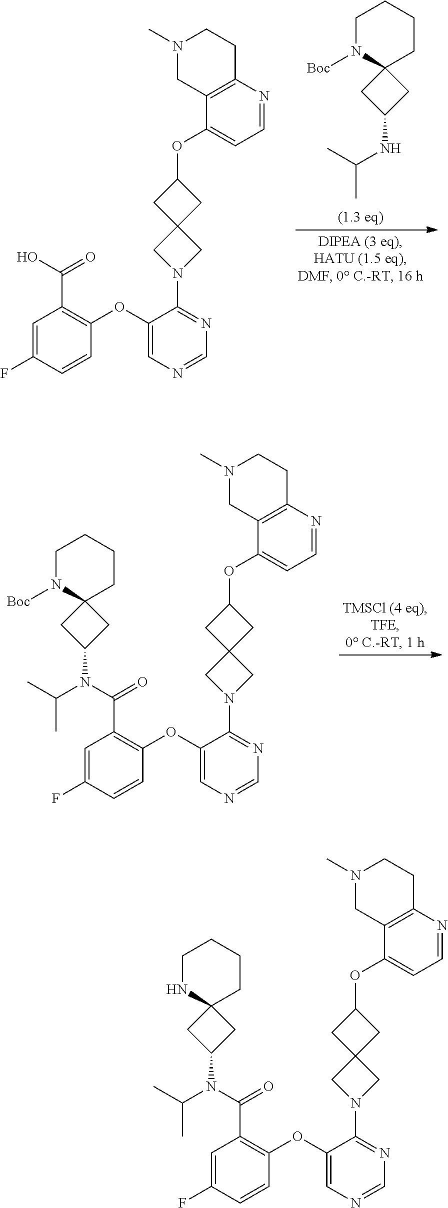 [SMILES: CN1CCc2nccc(OC3CC4(C3)CN(c3ncncc3Oc3ccc(F)cc3C(=O)O)C4)c2C1 ; CC(C)N[C@H]1C[C@@]2(CCCCN2C(=O)OC(C)(C)C)C1 ; CC(C)N(C(=O)c1cc(F)ccc1Oc1cncnc1N1CC2(CC(Oc3ccnc4c3CN(C)CC4)C2)C1)[C@H]1C[C@@]2(CCCCN2C(=O)OC(C)(C)C)C1 ; CC(C)N(C(=O)c1cc(F)ccc1Oc1cncnc1N1CC2(CC(Oc3ccnc4c3CN(C)CC4)C2)C1)[C@H]1C[C@@]2(CCCCN2)C1]

### Step 1. tert-Butyl(2s,4r)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.5]nonane-5-carboxylate

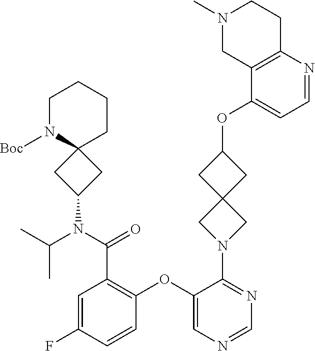 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCCN2C(=O)OC(C)(C)C)C1]

To a stirred solution of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid (600 mg, 1.221 mmol), tert-butyl(2s,4r)-2-(isopropylamino)-5-azaspiro[3.5]nonane-5-carboxylate (448 mg, 1.587 mmol) and DIPEA (0.640 mL, 3.66 mmol) in DMF (6 mL) was added HATU (696 mg, 1.831 mmol) at 0° C. The reaction was stirred at RT for 16 h, and monitored by LCMS. After completion, the reaction was diluted with water (30 mL) and extracted with EtOAc (3×20 mL). The combined organic layer was washed with brine (10 mL), dried over anhydrous sodium sulfate, and filtered, and the filtrate was concentrated under reduced pressure. The crude product was purified by silica-gel flash column chromatography using MeOH in DCM (product eluted at 19% MeOH in DCM) to obtain tert-butyl(2s,4r)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.5]nonane-5-carboxylate (620 mg, 44.8% yield) as a solid. LCMS (Method D): Rt=2.22 min, m/z=756.3 [M+H]⁺, 66.63%.

### Step 2. 5-Fluoro-N-isopropyl-2-((4-(6-((6-methyl-5,6,7,8-tetrahydro-, 6-naphthyridin-4-yl)oxy)-2-azaspiro[3.3]heptan-2-yl)pyrimidin-5-yl)oxy)-N-((2s,4r)-5-azaspiro[3.5]nonan-2-yl)benzamide

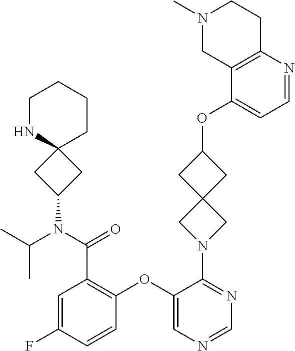 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCCN2)C1]

To a stirred solution of tert-butyl(2s,4r)-2-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-5-azaspiro[3.5]nonane-5-carboxylate (600 mg, 0.794 mmol) in TFE (6 mL) was added TMSCl (0.406 mL, 3.17 mmol) at 0° C. The reaction was stirred at RT for 1 h, and monitored by LCMS and TLC (10% MeOH in DCM). After completion, the reaction was concentrated under reduced pressure. The crude product was dissolved in DCM (5 mL) and TEA (0.5 mL) was added. The resulting reaction was stirred for 5 min, and then concentrated under reduced pressure. The crude product was purified by reverse phase chromatography using a SiliCycle SiliaSep C18 column (80 g) on Biotage isolera one (Mobile phase A: 0.1% NH₄HCO₃ in water, B: ACN). followed by Achiral prep SFC (Method I) to obtain 5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)-N-((2s,4r)-5-azaspiro[3.5]nonan-2-yl)benzamide (115 mg, 22% yield) as a solid. ¹H NMR (400 MHz, DMSO-d₆) δ 8.29-8.26 (m, 1H), 8.20-8.16 (m, 1H), 7.81-7.68 (m, 1H), 7.35-6.97 (m, 3H), 6.73-6.57 (m, 1H), 4.83-4.66 (m, 1H), 4.36-3.95 (m, 5H), 3.75-3.59 (m, 1H), 3.36 (s, 2H), 2.92-2.74 (m, 5H), 2.71-2.57 (m, 4H), 2.36 (s, 3H), 2.29-2.21 (m, 2H), 1.98-1.72 (m, 3H), 1.61-1.51 (m, 1H), 1.48-1.20 (m, 9H), 1.10-0.88 (m, 3H); LCMS (Method E): Rt=1.72 min, m/z=656.3 [M+H]⁺, 97.7%; HPLC (Method A): Rt=6.45 min, 99.66%; Chiral SFC (Method J): Rt=3.45 min, 100%.

### Example 235. N-((1r,3r)-3-Acetamido-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

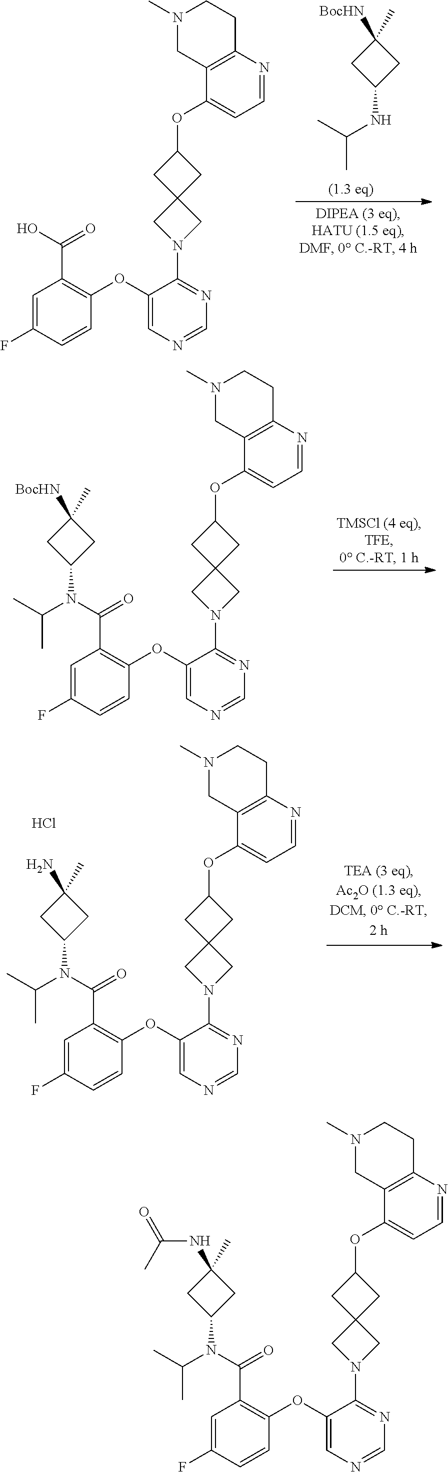

### Step 1. tert-Butyl (3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-1-methylcyclobutyl)carbamate

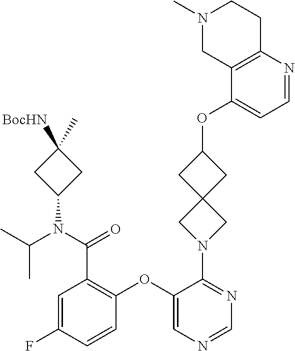 [SMILES: CC(C)N(C(=O)c1cc(F)ccc1Oc1cncnc1N1CC2(CC(Oc3ccnc4c3CN(C)CC4)C2)C1)[C@H]1C[C@](C)(NC(=O)OC(C)(C)C)C1]

To a stirred solution of 5-fluoro-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzoic acid (1 g, 2.034 mmol), tert-butyl ((1r,3r)-3-(isopropylamino)-1-methylcyclobutyl)carbamate (0.740 g, 3.05 mmol), and DIPEA (1.066 mL, 6.10 mmol) in DMF (10 mL) was added HATU (1.160 g, 3.05 mmol) at 0° C. The reaction was stirred at RT for 12 h, and monitored by LCMS and TLC (10% MeOH in DCM). After completion, the reaction was diluted with water (50 mL) and extracted with EtOAc (2×20 mL). The combined organic layer was washed with brine (20 mL), dried over anhydrous sodium sulfate, and filtered, and the filtrate was concentrated under reduced pressure. The crude was purified by reverse phase chromatography using a SiliCycle SiliaSep C18 column (80 g) on Biotage isolera one (mobile phase A: 0.1% NH₄HCO₃ in water, B: ACN) to obtain tert-butyl (3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-1-methylcyclobutyl)carbamate (250 mg, 16.48% yield) as a solid. LCMS (Method B): Rt=1.83 min, m/z=716.3 [M+H]⁺, 88.88%.

### Step 2. N-((1r,3r)-3-Amino-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide Hydrochloride

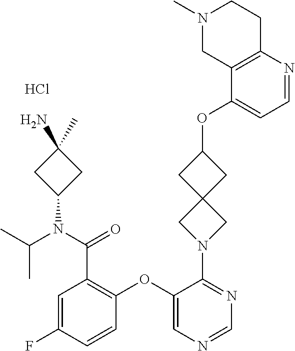 [SMILES: CC(C)N(C(=O)c1cc(F)ccc1Oc1cncnc1N1CC2(CC(Oc3ccnc4c3CN(C)CC4)C2)C1)[C@H]1C[C@](C)(N)C1 ; Cl]

To a stirred solution of tert-butyl ((1r,3r)-3-(5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamido)-1-methylcyclobutyl)carbamate (250 mg, 0.349 mmol) in TFE (5 mL) was added TMSCl (0.179 mL, 1.397 mmol) at 0° C. The reaction was stirred at RT 1 h, and monitored by LCMS and TLC (10% MeOH in DCM). After completion, the reaction was concentrated under reduced pressure to obtain N-((1r,3r)-3-amino-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide hydrochloride (230 mg, 88% yield) as a solid. This material was used without further purification. LCMS (Method B): Rt=0.815 min, m/z=616.4 [M+H]⁺, 70.29%.

### Step 3. N-((1r,3r)-3-Acetamido-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide

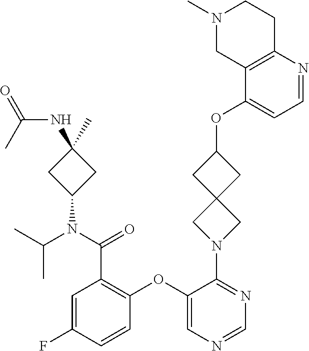 [SMILES: CC(=O)N[C@]1(C)C[C@@H](N(C(=O)c2cc(F)ccc2Oc2cncnc2N2CC3(CC(Oc4ccnc5c4CN(C)CC5)C3)C2)C(C)C)C1]

To a stirred solution of N-((1r,3r)-3-amino-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide hydrochloride (230 mg, 0.353 mmol) in DCM (5 mL), was added TEA (0.147 mL, 1.058 mmol), followed by dropwise addition of acetic anhydride (0.043 mL, 0.458 mmol) at 0° C. The reaction was stirred at RT for 2 h, and monitored by LCMS and TLC (10% MeOH in DCM). After completion, the reaction mixture was quenched with aqueous NaHCO₃ (20 mL) and extracted with 10% MeOH in DCM (2×20 mL). The combined organic layer was dried over anhydrous sodium sulfate and filtered, and the filtrate was concentrated under reduced pressure. The crude product was purified by reverse phase chromatography using a SiliCycle SiliaSep C18 column (80 g) on Biotage isolera one (Mobile phase A: 10 mM NH₄HCO₃ in water, B: ACN). followed by prep-HPLC purification (Method P) to obtain N-((1r,3r)-3-acetamido-3-methylcyclobutyl)-5-fluoro-N-isopropyl-2-((4-(6-((6-methyl-5,6,7,8-tetrahydro-1,6-naphthyridin-4-yl)oxy)-2-azaspiro[3.3]heptan-2-yl)pyrimidin-5-yl)oxy)benzamide (105 mg, 45.2% yield) as a solid. ¹H NMR (400 MHz, DMSO-d₆) δ 8.28 (s, 1H), 8.18 (d, J=1.0 Hz, 1H), 7.83 (d, J=1.0 Hz, 1H), 7.74 (d, J=1.0 Hz, 1H), 7.35-6.97 (m, 3H), 6.71-6.59 (m, 1H), 4.81-4.68 (m, 1H), 4.40-4.05 (m, 4H), 4.03-3.86 (m, 1H), 3.78-3.58 (m, 1H), 3.36 (s, 2H), 3.07-2.96 (m, 1H), 2.90-2.71 (m, 4H), 2.66-2.59 (m, 2H), 2.42-2.17 (m, 6H), 2.06-1.94 (m, 1H), 1.85-1.65 (m, 3H), 1.49-1.27 (m, 5H), 1.12-0.88 (m, 3H), two exchangeable protons merged with solvent peaks; LCMS (Method B): Rt=1.685 min, m/z=616.4 [M+H]⁺, 99.9%; HPLC (Method C): Rt=8.215 min, 99.81%.

### Example 236. 5-Fluoro-N-isopropyl-N-((1r,3r)-3-methyl-3-(pyrrolidin-1-yl)cyclobutyl)-2-((4-(6-((1,2,3,4-tetrahydroisoquinolin-8-yl)oxy)-2-azaspiro[3.3]heptan-2-yl)pyrimidin-5-yl)oxy)benzamide

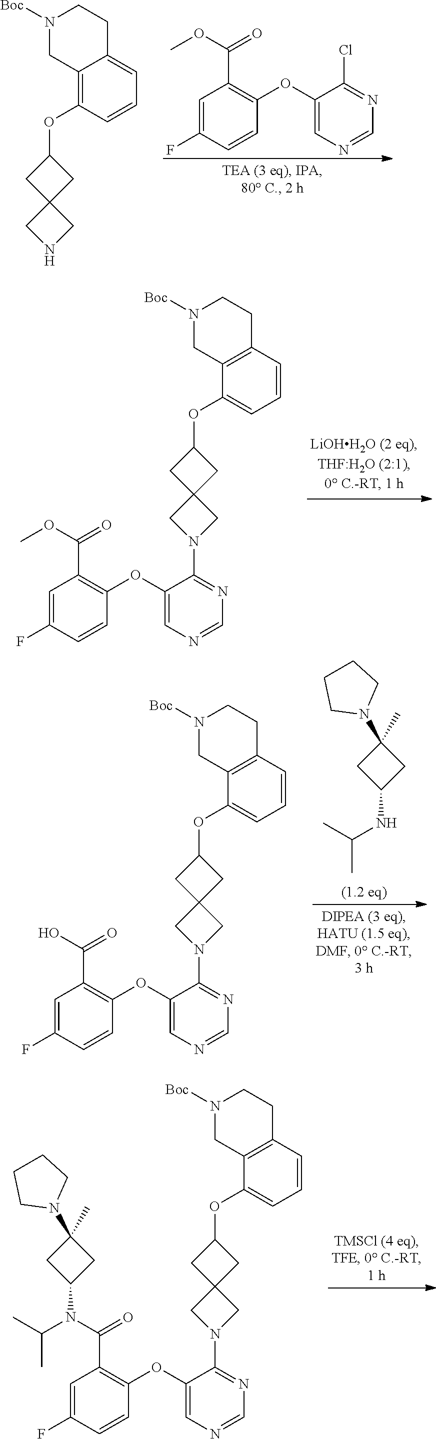

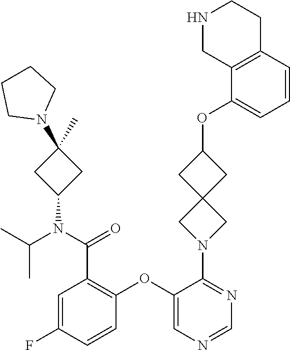 [SMILES: CC(C)N(C(=O)c1cc(F)ccc1Oc1cncnc1N1CC2(CC(Oc3cccc4c3CNCC4)C2)C1)[C@H]1C[C@](C)(N2CCCC2)C1]

### Step 1. tert-Butyl 8-((2-(S-(4-fluoro-2-(methoxycarbonyl)phenoxy)pyrimidin-4-yl)-2-azaspiro[3.3]heptan-6-yl)oxy)-3,4-dihydroisoquinoline-2(1H)-carboxylate

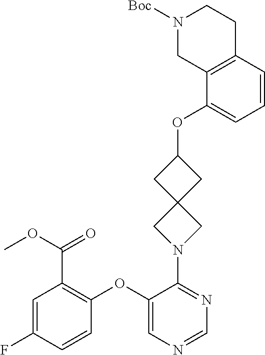 [SMILES: COC(=O)c1cc(F)ccc1Oc1cncnc1N1CC2(CC(Oc3cccc4c3CN(C(=O)OC(C)(C)C)CC4)C2)C1]

To a stirred solution of tert-butyl 8-((2-azaspiro[3.3]heptan-6-yl)oxy)-3,4-dihydroisoquinoline-2(1H)-carboxylate (2 g, 5.81 mmol) and TEA (2.448 mL, 17.42 mmol) in IPA (40 mL) was added methyl 2-((4-chloropyrimidin-5-yl)oxy)-5-fluorobenzoate (1.641 g, 5.81 mmol) at RT. The reaction was heated at 80° C. for 2 h, with monitoring by LCMS and TLC (10% MeOH in DCM). After completion, the reaction was concentrated under reduced pressure. The residue was diluted with water (50 mL) and the mixture was extracted with DCM (2×20 mL). The combined organic layer was dried over anhydrous sodium sulfate and filtered, and the filtrate was concentrated under reduced pressure. The crude product was purified by silica-gel flash column chromatography using MeOH in DCM (product eluted at 3% MeOH in DCM) to obtain tert-butyl 8-((2-(5-(4-fluoro-2-(methoxycarbonyl)phenoxy)pyrimidin-4-yl)-2-azaspiro[3.3]heptan-6-yl)oxy)-3,4-dihydroisoquinoline-2(1H)-carboxylate (3.0 g, 77% yield) as a solid. LCMS (Method A): Rt=2.576 min, m/z=591.3 [M+H]⁺, 87.85%.

### Step 2. ((4-(6-((2-(tert-Butoxycarbonyl)-1,2,3,4-tetrahydroisoquinolin-8-yl)oxy)-2-azaspiro[3.3]heptan-2-yl)pyrimidin-5-yl)oxy)-5-fluorobenzoic Acid

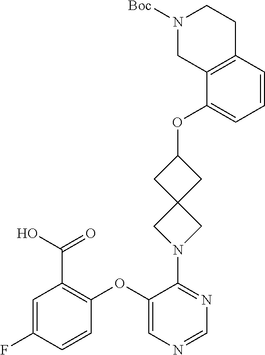 [SMILES: CC(C)(C)OC(=O)N1CCc2cccc(OC3CC4(C3)CN(c3ncncc3Oc3ccc(F)cc3C(=O)O)C4)c2C1]

To a stirred solution of tert-butyl 8-((2-(5-(4-fluoro-2-(methoxycarbonyl)phenoxy)pyrimidin-4-yl)-2-azaspiro[3.3]heptan-6-yl)oxy)-3,4-dihydroisoquinoline-2(1H)-carboxylate (3 g, 5.08 mmol) in THF (30 mL) and water (15 mL) was added LiOH·H₂O (0.426 g, 10.16 mmol) at 0° C. The reaction was stirred at RT for 1 h, with monitoring by LCMS and TLC (10% MeOH in hexane). After completion, the reaction was concentrated under reduced pressure. The residue was acidified to pH=4-5 with 1.5N HCl (5 mL) and the mixture was extracted with 10% MeOH in DCM (3×20 mL). The combined organic layer was dried over anhydrous sodium sulfate and filtered, and the filtrate was concentrated under reduced pressure to obtain 2-((4-(6-((2-(tert-butoxycarbonyl)-1,2,3,4-tetrahydroisoquinolin-8-yl)oxy)-2-azaspiro[3.3]heptan-2-yl)pyrimidin-5-yl)oxy)-5-fluorobenzoic acid (2.7 g, 80% yield) as a solid. This material was used without further purification. LCMS (Method D): Rt=1.611 min, m/z=577.3 [M+H]⁺, 86.78%.

### Step-3. tert-Butyl 8-((2-(S-(4-fluoro-2-(isopropyl((1r,3r)-3-methyl-3-(pyrrolidin-1-yl)cyclobutyl)carbamoyl)phenoxy)pyrimidin-4-yl)-2-azaspiro[3.3]heptan-6-yl)oxy)-3,4-dihydroisoquinoline-2(1H)-carboxylate

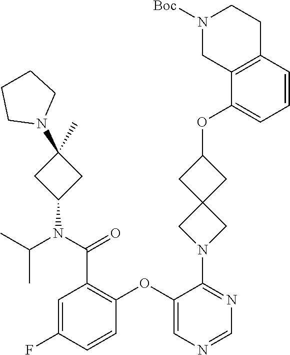 [SMILES: CC(C)N(C(=O)c1cc(F)ccc1Oc1cncnc1N1CC2(CC(Oc3cccc4c3CN(C(=O)OC(C)(C)C)CC4)C2)C1)[C@H]1C[C@](C)(N2CCCC2)C1]

To a stirred solution of 2-((4-(6-((2-(tert-butoxycarbonyl)-1,2,3,4-tetrahydroisoquinolin-8-yl)oxy)-2-azaspiro[3.3]heptan-2-yl)pyrimidin-5-yl)oxy)-5-fluorobenzoic acid (1.6 g, 2.77 mmol) and (1r,3r)—N-isopropyl-3-methyl-3-(pyrrolidin-1-yl)cyclobutan-1-amine (0.654 g, 3.33 mmol) in DMF (16 mL) was added DIPEA (1.454 mL, 8.32 mmol) and HATU (1.583 g, 4.16 mmol) at 0° C. The reaction was stirred at RT for 3 h, and monitored by TLC (10% MeOH in DCM). After completion, the reaction was diluted with water (25 mL) and extracted with EtOAc (2×25 mL). The combined organic layer was washed with brine (25 mL), dried over anhydrous sodium sulfate and filtered, and the filtrate was concentrated under reduced pressure. The crude product was purified by reverse phase chromatography using a SiliCycle SiliaSep C18 column (80 g) on Biotage isolera one (mobile phase A: 0.1% ABC in water, B: ACN) to obtain tert-butyl 8-((2-(5-(4-fluoro-2-(isopropyl((1r,3r)-3-methyl-3-(pyrrolidin-1-yl)cyclobutyl)carbamoyl)phenoxy)pyrimidin-4-yl)-2-azaspiro[3.3]heptan-6-yl)oxy)-3,4-dihydroisoquinoline-2(1H)-carboxylate (0.9 g, 40.0% yield) as a solid. LCMS (Method B): Rt=1.74 min, m/z=755.4 [M+H]⁺, 93.11%.

### Step-4. 5-Fluoro-N-isopropyl-N-((1r,3r)-3-methyl-3-(pyrrolidin-1-yl)cyclobutyl)-2-((4-(6-((1,2,3,4-tetrahydroisoquinolin-8-yl)oxy)-2-azaspiro[3.3]heptan-2-yl)pyrimidin-5-yl)oxy)benzamide

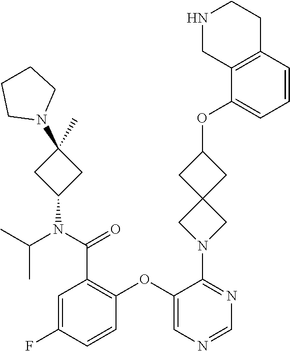 [SMILES: CC(C)N(C(=O)c1cc(F)ccc1Oc1cncnc1N1CC2(CC(Oc3cccc4c3CNCC4)C2)C1)[C@H]1C[C@](C)(N2CCCC2)C1]

To a stirred solution of tert-butyl 8-((2-(5-(4-fluoro-2-(isopropyl((1r,3r)-3-methyl-3-(pyrrolidin-1-yl)cyclobutyl)carbamoyl)phenoxy)pyrimidin-4-yl)-2-azaspiro[3.3]heptan-6-yl)oxy)-3,4-dihydroisoquinoline-2(1H)-carboxylate (0.9 g, 1.192 mmol) in TFE (10 mL) was added TMSCl (0.609 mL, 4.77 mmol) at 0° C. The reaction was stirred at RT for 1 h, and monitored by TLC (10% MeOH in DCM). After completion, the reaction was concentrated under reduced pressure. The crude product was dissolved in 10% MeOH in DCM (20 mL) and basified with aqueous NaHCO₃ solution (20 mL). The aqueous layer was extracted with 10% MeOH in DCM (2×20 mL). The combined organic layer was dried over anhydrous sodium sulfate and filtered, and the filtrate was concentrated under reduced pressure to obtain crude (750 mg, 96% yield) as a solid. A portion of the crude product (250 mg) was purified by reverse phase chromatography using a SiliCycle SiliaSep C18 column (80 g) on Biotage isolera one (mobile phase A: 0.1% ABC in water, B: ACN) to obtain 5-fluoro-N-isopropyl-N-((1r,3r)-3-methyl-3-(pyrrolidin-1-yl)cyclobutyl)-2-((4-(6-((1,2,3,4-tetrahydroisoquinolin-8-yl)oxy)-2-azaspiro[3.3]heptan-2-yl)pyrimidin-5-yl)oxy)benzamide (72 mg) as a solid. ¹H NMR (400 MHz, DMSO-d₆) δ 8.27 (d, J=2.9 Hz, 1H), 7.80-7.57 (m, 1H), 7.34-6.95 (m, 4H), 6.70-6.48 (m, 2H), 4.60 (t, J=6.5 Hz, 1H), 4.40-4.00 (m, 4H), 3.90-3.61 (m, 4H), 2.91-2.59 (m, 6H), 2.57-2.53 (m, 2H), 2.41-2.33 (m, 2H), 2.28-2.07 (m, 4H), 2.03-1.90 (m, 1H), 1.85-1.75 (m, 1H), 1.73-1.54 (m, 3H), 1.49-1.39 (m, 2H), 1.36-1.29 (m, 2H), 1.28-1.20 (m, 2H), 1.13-0.87 (m, 5H); LCMS (Method B): Rt=1.196 min, m/z=655.4 [M+H]⁺, 99.71%; HPLC (Method H): Rt=6.294 min, 99.87%. Chiral SFC (Method Y): Rt=4.53 min, 99.14%.

Note: The remaining 500 mg of this material was used without further purification.

### Example 237: 5-Fluoro-N-isopropyl-2-((4-(6-((2-methyl-1,2,3,4-tetrahydroisoquinolin-8-yl)oxy)-2-azaspiro[3.3]heptan-2-yl)pyrimidin-5-yl)oxy)-N-(3-methyl-3-(pyrrolidin-1-yl)cyclobutyl)benzamide

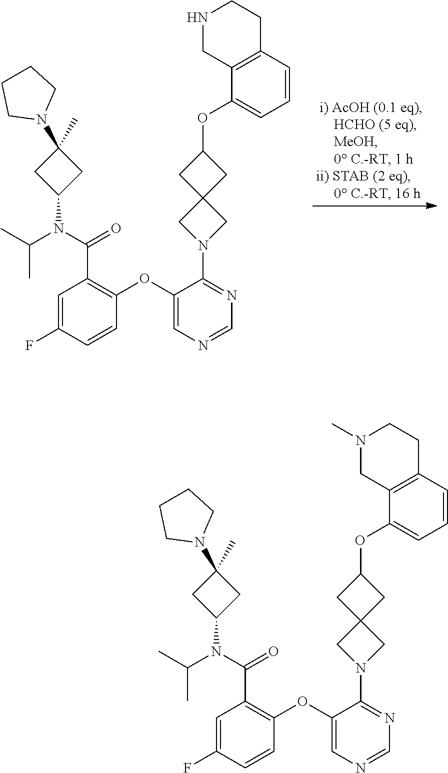 [SMILES: CC(C)N(C(=O)c1cc(F)ccc1Oc1cncnc1N1CC2(CC(Oc3cccc4c3CNCC4)C2)C1)[C@H]1C[C@](C)(N2CCCC2)C1 ; CC(C)N(C(=O)c1cc(F)ccc1Oc1cncnc1N1CC2(CC(Oc3cccc4c3CN(C)CC4)C2)C1)[C@H]1C[C@](C)(N2CCCC2)C1]

### Step 1. 5-Fluoro-N-isopropyl-2-((4-(6-((2-methyl-1,2,3,4-tetrahydroisoquinolin-8-yl)oxy)-2-azaspiro[3.3]heptan-2-yl)pyrimidin-5-yl)oxy)-N-(3-methyl-3-(pyrrolidin-1-yl)cyclobutyl)benzamide

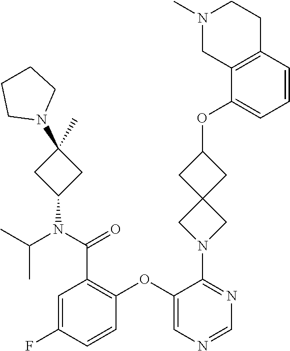 [SMILES: CC(C)N(C(=O)c1cc(F)ccc1Oc1cncnc1N1CC2(CC(Oc3cccc4c3CN(C)CC4)C2)C1)[C@H]1C[C@](C)(N2CCCC2)C1]

To a stirred solution of 5-fluoro-N-isopropyl-N-((1r,3r)-3-methyl-3-(pyrrolidin-1-yl)cyclobutyl)-2-((4-(6-((1,2,3,4-tetrahydroisoquinolin-8-yl)oxy)-2-azaspiro[3.3]heptan-2-yl)pyrimidin-5-yl)oxy)benzamide (500 mg, 0.764 mmol) in MeOH (6 mL) was added formaldehyde (37% in H₂O, 0.284 mL, 3.82 mmol) and AcOH (4.37 μL, 0.076 mmol) at 0° C. The reaction was stirred at RT for 1 h then cooled to 0° C., and NaBH(OAc)₃ (324 mg, 1.527 mmol) was added. The resulting reaction was stirred at RT for 16 h, and monitored by LCMS. After completion, the reaction was concentrated under reduced pressure. The crude product was dissolved in 10% MeOH in DCM (20 mL) and the solution was basified with aqueous NaHCO₃ solution (20 mL). The aqueous layer was extracted with 10% MeOH in DCM (2×20 mL). The combined organic layer was dried over anhydrous sodium sulfate and filtered, and the filtrate was concentrated under reduced pressure. The crude product was purified by reverse phase chromatography using a SiliCycle SiliaSep C18 column (80 g) on Biotage isolera one (mobile phase A: 0.1% FA in water, B: ACN) to obtain the desired product as a formate salt. The formate salt was basified with aqueous NaHCO₃ solution (20 mL) and extracted with 10% MeOH in DCM (2×20 mL). The combined organic layer was dried over anhydrous sodium sulfate and filtered, and the filtrate was concentrated under reduced pressure to obtain 5-fluoro-N-isopropyl-2-((4-(6-((2-methyl-1,2,3,4-tetrahydroisoquinolin-8-yl)oxy)-2-azaspiro[3.3]heptan-2-yl)pyrimidin-5-yl)oxy)-N-(3-methyl-3-(pyrrolidin-1-yl)cyclobutyl)benzamide (350 mg, 68.4% yield) as a solid. ¹H NMR (400 MHz, DMSO-d₆) δ 8.34-8.21 (m, 1H), 7.85-7.55 (m, 1H), 7.32-6.95 (m, 4H), 6.68 (d, J=7.5 Hz, 1H), 6.61-6.50 (m, 1H), 4.69-4.56 (m, 1H), 4.36-4.00 (m, 5H), 3.91-3.79 (m, 1H), 3.75-3.63 (m, 1H), 3.34 (s, 2H), 2.89-2.65 (m, 5H), 2.59-2.52 (m, 4H), 2.34 (s, 3H), 2.27-2.15 (m, 3H), 2.03-1.75 (m, 2H), 1.73-1.54 (m, 4H), 1.49-1.29 (m, 3H), 1.26 (s, 2H), 1.10-0.91 (m, 5H); LCMS (Method B): Rt=1.09 min, m/z=669.4 [M+H]⁺, 98.71%; HPLC (Method H): Rt=6.31 min, 99.86%. Chiral SFC (Method P): Rt=3.84 min, 99.23%.

### Example 238. Menin-MLL Competition and MV4; 11 Cell Proliferation Assays

Menin-MLL is a competition assay between human Menin and N-terminal portion of human MLL representing amino acids 4-43 of the protein. The interaction between Menin and MLL peptide was monitored by HTRF employing Terbium labeled anti-His6 antibody directed to the N-terminal His6-tag on recombinant Menin and FITC group covalently attached to the MLL peptide. The N-terminal fragment of MLL, retained in all MLL fusion proteins, is involved in the interactions with Menin, and this protein-protein interaction is critical for the MLL fusion proteins mediated leukemogenic transformations.

For IC₅₀ determination test compounds were prepared as 10 mM DMSO stock solutions. Considering DMSO as the vehicle in the assay system. Lower sub-stocks of 50 μM were prepared from the 10 mM stock solution. To test the compounds in assay, 3.16-fold serial dilutions are made in 100% DMSO. Mid-stock of 50× compounds (50 μM) were serially diluted (3.16 fold) in 100%DMSO in Polypropylene plate. In assay plate 1 micro-litre of the previously prepared compound dilution was stamped. H-FL-Menin diluted to 4 nM in assay buffer (50 mM Tris-HCl, pH 7.4, 50 mM NaCl, freshly prepared 1 mM DTT, 0.01% BSA, 0.005% Triton X-100) was pre-incubated with 8 nM anti-His6-Tb for 30 min at room temperature. FITC-MLL-4-43 was diluted to 2 nM in assay buffer and 25 μL was dispensed into each well of the assay plate followed by addition of 25 μl of pre-incubated H-FL-Menin and anti-His6-Tb mixture. Final concentration H-FL-Menin diluted to 1 nM in assay plate with 2 nM anti-His6-Tb and 1 nM FITC-MLL-4-43. After 1 hr incubation at room temperature, the HTRF signal was measured on the Spark multi-label plate reader. Resulting data were captured as a ratio of RFU520/RFU485×1000. The max values were obtained from 0% inhibition in presence of 2% DMSO and the min. values were 100% inhibition in presence of 1 μM reference compound.

### Example 239. Cell Proliferation Assay on MV4; 11 and MV4-11 [M327I-Heterozygous] Cells Using CTG Assay

### Test Compounds Preparation

Compounds were dissolved to obtain as 10 mM solution in DMSO. These stocks were diluted 1:5 to the top concentration to achieve 2 mM in 100% DMSO. For EC₅₀ determination, compounds were serially diluted with semi-log in 100% DMSO. Each prepared DMSO solution were further diluted 1:500 in the cell culture media to obtain the 2× dosing solutions. The Final concentrations of tested compounds in the cell culture media ranged from 0.632 nM to 2000 nM which keeps % DMSO in assay as 0.1 across the wells.

### Experimental Procedure

MV4:11 [harboring the MLL-AF4; (ATCC Cat. No. CRL-9591)]& mutated cell line of MV4-11 [M327I-hetero; Source: DFCI, Boston, MA] cells were cultured in IMDM/RPMI with 10% HI FBS at 5% CO₂ and 37° C. The cell suspension containing 150,000 cells/mL in the culture medium was dispensed in 96-well cell culture plates 100 μL (15,000 cell/well) per well. Then 100 μL of 2× dosing media with test compounds were added to each well. Cells were cultured for 72 h at 37° C. and 5% CO₂ in a humidified incubator.

Post 72 h incubation, cultured cells were resuspended and 100 μL of the suspension from the assay plate was transferred into 96-well black OptiPlate (PerkinElmer Cat. No. 6005320). To this, 100 μL of Cell Titer Glo reagent was added, assay plates were shaken for 5 min at RT, and luminescence was read on Tecan Spark (Tecan) plate reader.

### Data Analysis

Percent inhibition values were calculated based on the high luminescence signal obtained with 0.1% DMSO control and the low signal observed with 5 μM Revumenib. The percent inhibition vs. concentration data were fit into a four-parameter model, and EC₅₀ values were calculated from the fit as the concentrations corresponding to the inflection points on the dose-response curves. Data is shown in Table 2.

### Example 240. Determination of Binding Constants to Menin-MLL

For Ki determination, individual compounds were prepared as 10 mM DMSO stock solutions. Considering DMSO as the vehicle in the assay system. Lower sub-stocks of 16 μM were prepared from the 10 mM stock solution. To test the compounds in assay, 3.16-fold serial dilutions are made in 100% DMSO. Mid-stock of 50× compounds (16 μM) were serially diluted (3.16 fold) in 100% DMSO in Polypropylene plate. In assay plate 1 micro-litre of the previously prepared compound dilution was stamped. H-FL-Menin diluted to 1 nM in assay buffer (50 mM Tris-HCl, pH 7.4, 50 mM NaCl, freshly prepared 1 mM DTT, 0.01% BSA, 0.005% Triton X-100) and pre-incubated with 2 nM anti-His6-Tb for 30 min at room temperature and then 25 μl was dispensed into each well. FITC-MLL-4-43 was diluted to 6.4 nM in assay buffer and 25 μL was dispensed into each well of the assay plate followed by addition of 25 μl of pre-incubated H-FL-Menin and anti-His6-Tb mixture. Final concentration H-FL-Menin diluted to 0.25 nM in assay plate with 0.5 nM anti-His6-Tb and 3.2 nM FITC-MLL-4-43. After 24 hr incubation at room temperature, the HTRF signal was measured on the Spark multi-label plate reader. Resulting data were captured as a ratio of RFU520/RFU485×1000. The max values were obtained from 0% inhibition in presence of 2% DMSO and the min. values were 100% inhibition in presence of 320 nM reference compound. Data is shown in Table 2.

Table 2. Shows (i) Menin-MLL binding data according to Homogeneous Time Resolved Fluorescence (HTRF) assay with (i) individual compound Ki (nM) and (ii) the EC₅₀ (nM) against MV4; 11 and MV4; 11 M327I leukemia cell lines. Ki data (nM) are provided below (“n/a” refers to data not available; “+++” means <1.0 nM; “++” means ≥1.0 nM and <1.5 nM; and “+” means ≥1.5 nM). EC₅₀ (nM) against MV4; 11 and MV4; 11 M327I data are provided below (“n/a” refers to data not available; “+++” means <100 nM; “++” means ≥100 nM and <1000 nM; and “+” means ≥1000 nM).

| | | WT | MV4;11 | MV4;11 |
|---|---|---|---|---|
| Compound | | Menin | EC₅₀ | M3271 (het) |
| No. | Structure | Ki (nM) | (nM) | EC₅₀ (nM) |
| 1 | 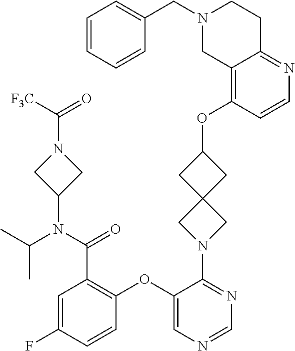 [SMILES: CC(C)N(C(=O)c1cc(F)ccc1Oc1cncnc1N1CC2(CC(Oc3ccnc4c3CN(Cc3ccccc3)CC4)C2)C1)C1CN(C(=O)C(F)(F)F)C1] | +++ | +++ | +++ |
| 2 | 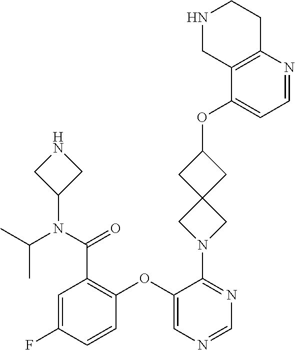 [SMILES: CC(C)N(C(=O)c1cc(F)ccc1Oc1cncnc1N1CC2(CC(Oc3ccnc4c3CNCC4)C2)C1)C1CNC1] | +++ | ++ | ++ |
| 3 | 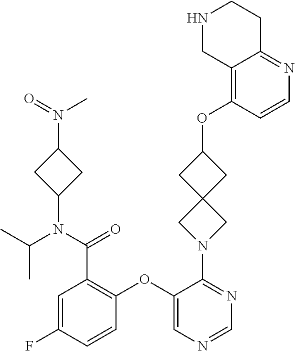 | +++ | ++ | ++ |
| 4 | 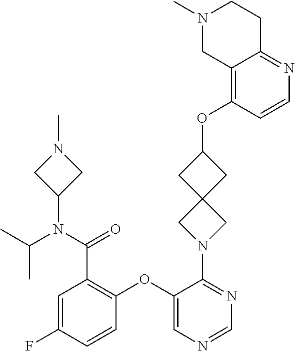 [SMILES: CC(C)N(C(=O)c1cc(F)ccc1Oc1cncnc1N1CC2(CC(Oc3ccnc4c3CN(C)CC4)C2)C1)C1CN(C)C1] | +++ | +++ | +++ |
| 5 | 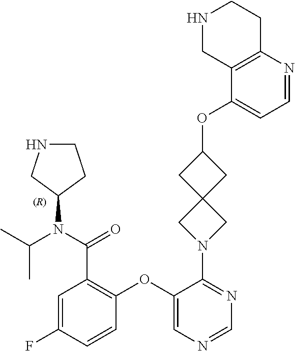 | +++ | ++ | ++ |
| 6 | 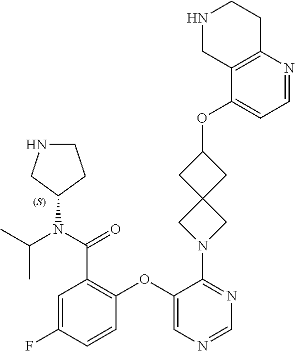 | +++ | +++ | ++ |
| 7 | 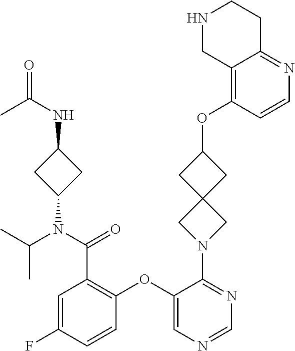 [SMILES: CC(=O)N[C@H]1C[C@H](N(C(=O)c2cc(F)ccc2Oc2cncnc2N2CC3(CC(Oc4ccnc5c4CNCC5)C3)C2)C(C)C)C1] | +++ | ++ | ++ |
| 8 | 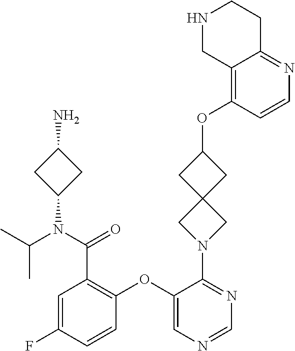 [SMILES: CC(C)N(C(=O)c1cc(F)ccc1Oc1cncnc1N1CC2(CC(Oc3ccnc4c3CNCC4)C2)C1)[C@H]1C[C@@H](N)C1] | +++ | +++ | +++ |
| 9 | 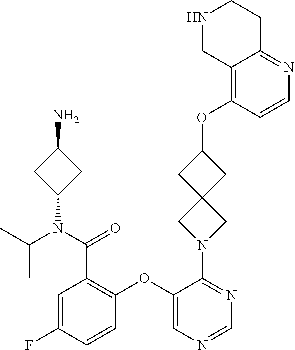 [SMILES: CC(C)N(C(=O)c1cc(F)ccc1Oc1cncnc1N1CC2(CC(Oc3ccnc4c3CNCC4)C2)C1)[C@H]1C[C@H](N)C1] | +++ | +++ | +++ |
| 10 | 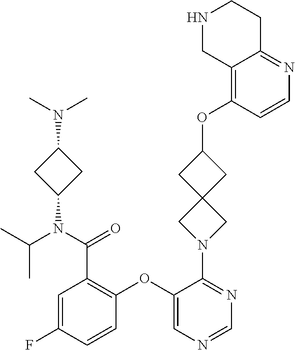 [SMILES: CC(C)N(C(=O)c1cc(F)ccc1Oc1cncnc1N1CC2(CC(Oc3ccnc4c3CNCC4)C2)C1)[C@H]1C[C@@H](N(C)C)C1] | +++ | +++ | +++ |
| 11 | 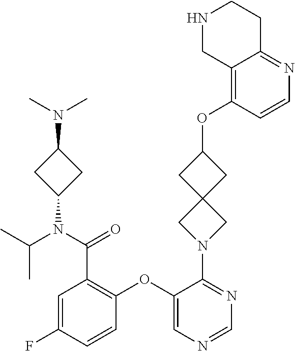 [SMILES: CC(C)N(C(=O)c1cc(F)ccc1Oc1cncnc1N1CC2(CC(Oc3ccnc4c3CNCC4)C2)C1)[C@H]1C[C@H](N(C)C)C1] | +++ | +++ | +++ |
| 12 | 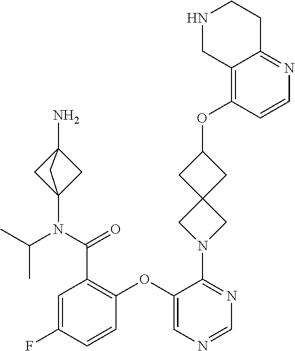 [SMILES: CC(C)N(C(=O)c1cc(F)ccc1Oc1cncnc1N1CC2(CC(Oc3ccnc4c3CNCC4)C2)C1)C12CC(N)(C1)C2] | +++ | +++ | +++ |
| 13 | 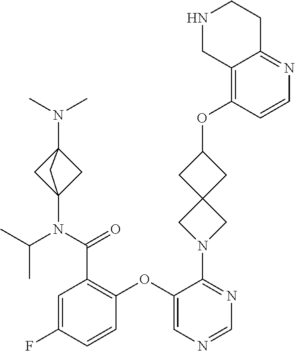 [SMILES: CC(C)N(C(=O)c1cc(F)ccc1Oc1cncnc1N1CC2(CC(Oc3ccnc4c3CNCC4)C2)C1)C12CC(N(C)C)(C1)C2] | +++ | +++ | +++ |
| 14 | 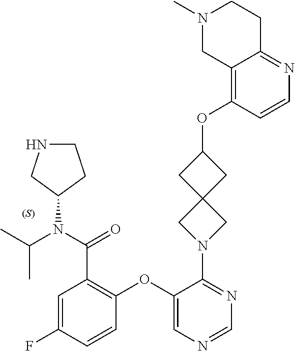 | +++ | +++ | +++ |
| 15 | 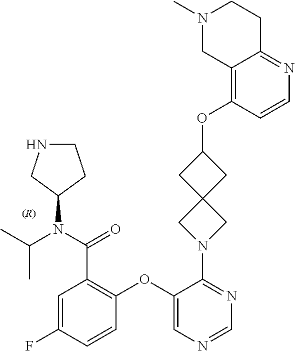 | +++ | +++ | +++ |
| 16 | 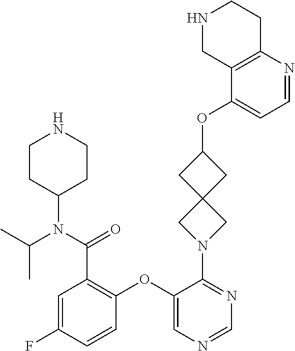 [SMILES: CC(C)N(C(=O)c1cc(F)ccc1Oc1cncnc1N1CC2(CC(Oc3ccnc4c3CNCC4)C2)C1)C1CCNCC1] | +++ | ++ | ++ |
| 17 | 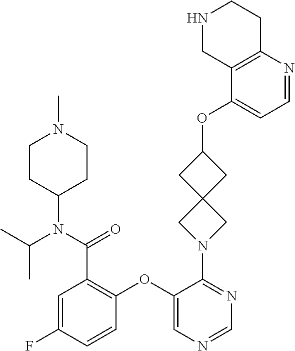 [SMILES: CC(C)N(C(=O)c1cc(F)ccc1Oc1cncnc1N1CC2(CC(Oc3ccnc4c3CNCC4)C2)C1)C1CCN(C)CC1] | +++ | +++ | +++ |
| 18 | 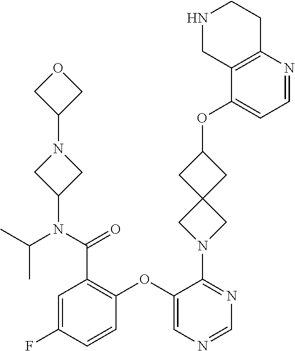 [SMILES: CC(C)N(C(=O)c1cc(F)ccc1Oc1cncnc1N1CC2(CC(Oc3ccnc4c3CNCC4)C2)C1)C1CN(C2COC2)C1] | +++ | +++ | +++ |
| 19 | 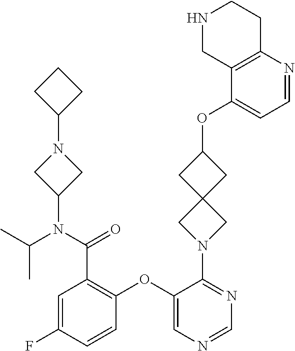 [SMILES: CC(C)N(C(=O)c1cc(F)ccc1Oc1cncnc1N1CC2(CC(Oc3ccnc4c3CNCC4)C2)C1)C1CN(C2CCC2)C1] | +++ | +++ | +++ |
| 20 | 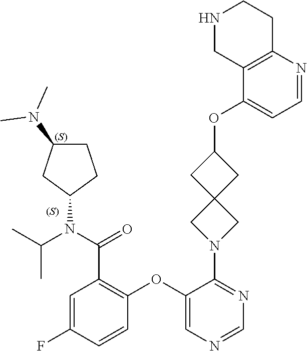 | +++ | +++ | +++ |
| 21 | 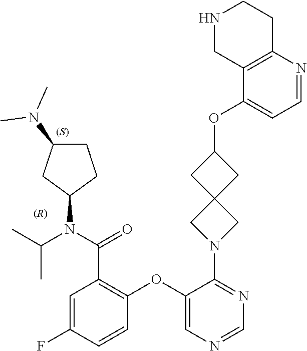 | +++ | +++ | ++ |
| 22 | 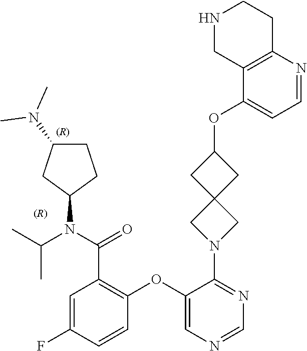 | +++ | +++ | +++ |
| 23 | 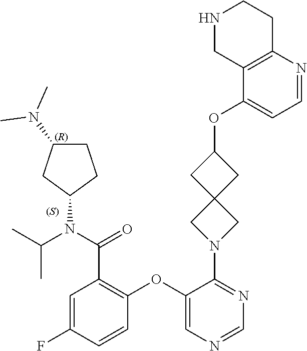 | +++ | +++ | +++ |
| 24 | 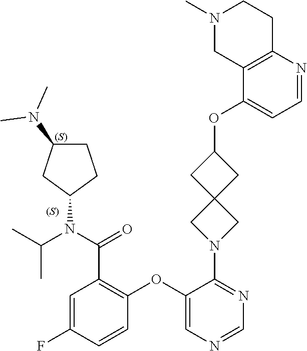 | +++ | +++ | +++ |
| 25 | 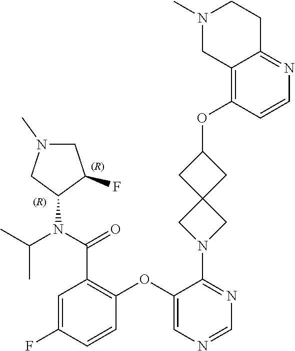 | +++ | +++ | +++ |
| 26 | 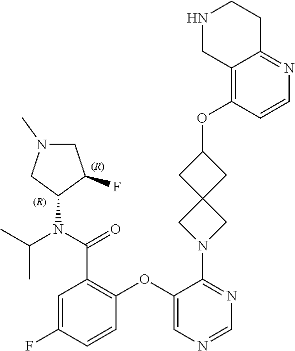 | +++ | +++ | +++ |
| 27 | 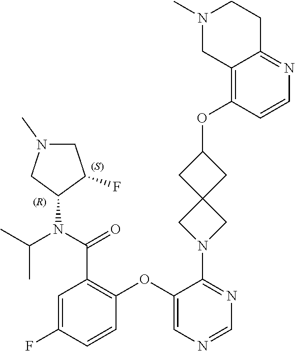 | +++ | +++ | +++ |
| 28 | 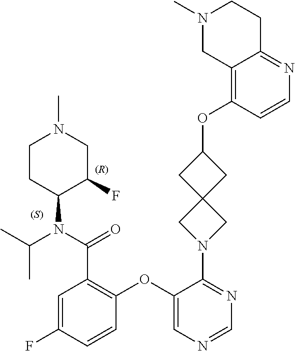 | +++ | +++ | +++ |
| 29 | 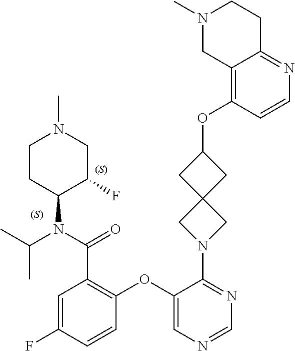 | +++ | +++ | +++ |
| 30 | 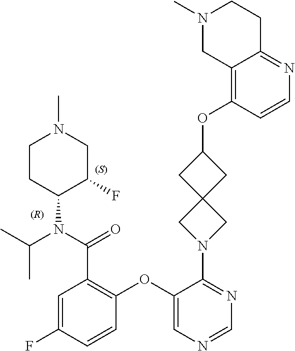 | +++ | +++ | +++ |
| 31 | 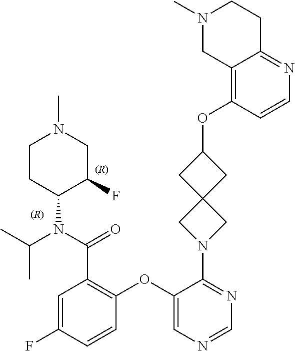 | +++ | +++ | +++ |
| 32 | 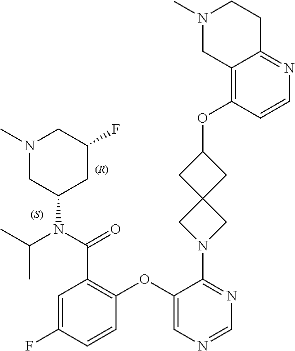 | +++ | +++ | +++ |
| 33 | 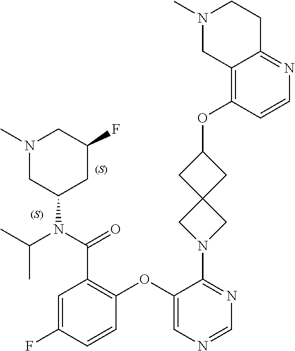 | +++ | +++ | +++ |
| 34 | 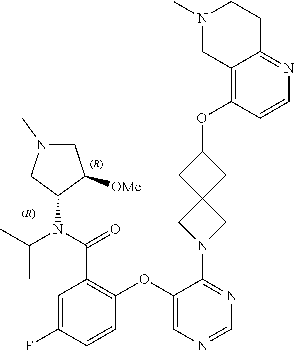 | +++ | +++ | +++ |
| 35 | 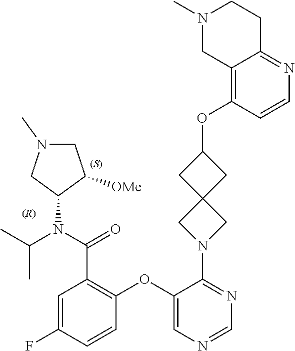 | +++ | +++ | +++ |
| 36 | 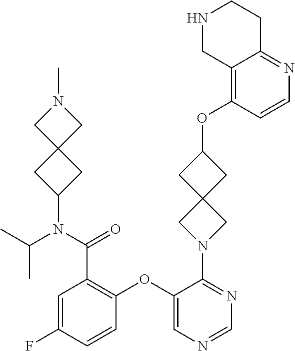 [SMILES: CC(C)N(C(=O)c1cc(F)ccc1Oc1cncnc1N1CC2(CC(Oc3ccnc4c3CNCC4)C2)C1)C1CC2(C1)CN(C)C2] | +++ | +++ | ++ |
| 37 | 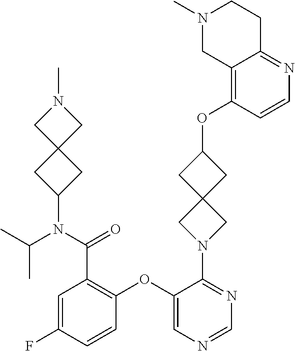 [SMILES: CC(C)N(C(=O)c1cc(F)ccc1Oc1cncnc1N1CC2(CC(Oc3ccnc4c3CN(C)CC4)C2)C1)C1CC2(C1)CN(C)C2] | +++ | +++ | +++ |
| 38 | 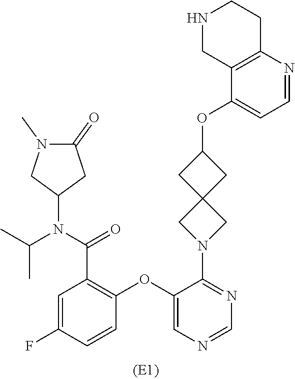 [SMILES: CC(C)N(C(=O)c1cc(F)ccc1Oc1cncnc1N1CC2(CC(Oc3ccnc4c3CNCC4)C2)C1)C1CC(=O)N(C)C1] | +++ | ++ | ++ |
| 39 | 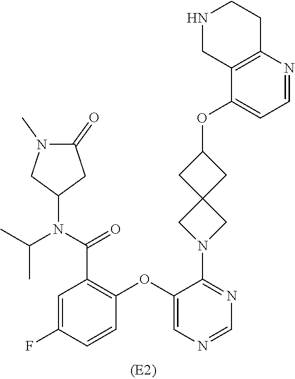 [SMILES: CC(C)N(C(=O)c1cc(F)ccc1Oc1cncnc1N1CC2(CC(Oc3ccnc4c3CNCC4)C2)C1)C1CC(=O)N(C)C1] | +++ | ++ | ++ |
| 40 | 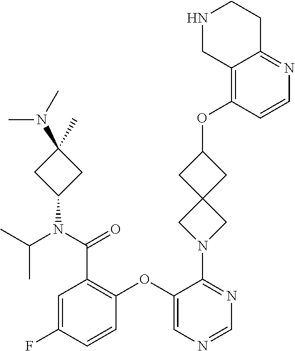 [SMILES: CC(C)N(C(=O)c1cc(F)ccc1Oc1cncnc1N1CC2(CC(Oc3ccnc4c3CNCC4)C2)C1)[C@H]1C[C@](C)(N(C)C)C1] | +++ | +++ | +++ |
| 41 | 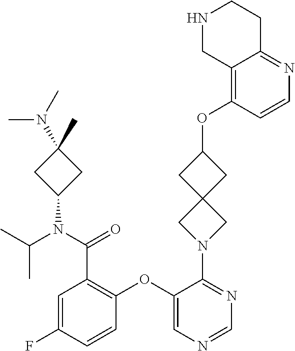 [SMILES: CC(C)N(C(=O)c1cc(F)ccc1Oc1cncnc1N1CC2(CC(Oc3ccnc4c3CNCC4)C2)C1)[C@H]1C[C@@](C)(N(C)C)C1] | +++ | +++ | +++ |
| 42 | 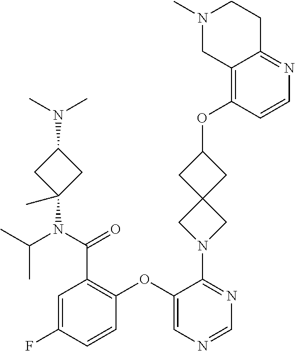 [SMILES: CC(C)N(C(=O)c1cc(F)ccc1Oc1cncnc1N1CC2(CC(Oc3ccnc4c3CN(C)CC4)C2)C1)[C@]1(C)C[C@H](N(C)C)C1] | +++ | +++ | +++ |
| 43 | 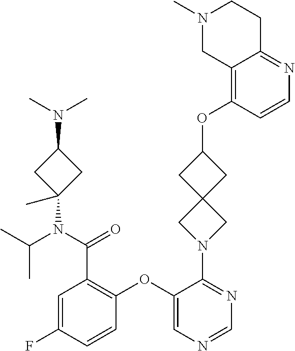 [SMILES: CC(C)N(C(=O)c1cc(F)ccc1Oc1cncnc1N1CC2(CC(Oc3ccnc4c3CN(C)CC4)C2)C1)[C@]1(C)C[C@@H](N(C)C)C1] | +++ | +++ | +++ |
| 44 | 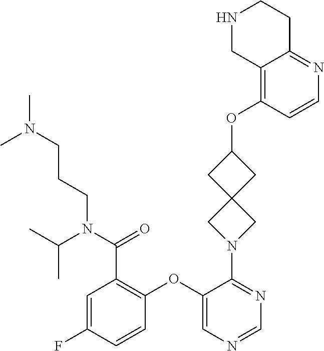 [SMILES: CC(C)N(CCCN(C)C)C(=O)c1cc(F)ccc1Oc1cncnc1N1CC2(CC(Oc3ccnc4c3CNCC4)C2)C1] | +++ | +++ | +++ |
| 45 | 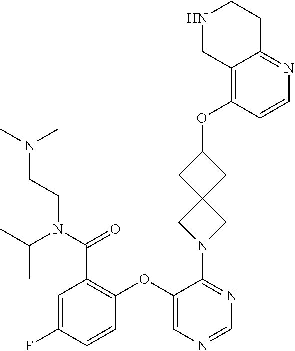 [SMILES: CC(C)N(CCN(C)C)C(=O)c1cc(F)ccc1Oc1cncnc1N1CC2(CC(Oc3ccnc4c3CNCC4)C2)C1] | +++ | +++ | +++ |
| 46 | 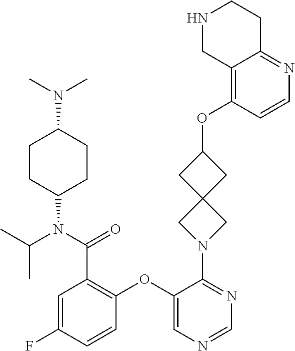 | +++ | +++ | +++ |
| 47 | 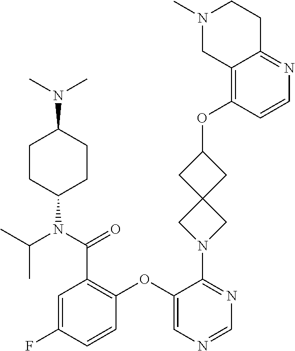 [SMILES: CC(C)N(C(=O)c1cc(F)ccc1Oc1cncnc1N1CC2(CC(Oc3ccnc4c3CN(C)CC4)C2)C1)[C@H]1CC[C@H](N(C)C)CC1] | +++ | +++ | +++ |
| 48 | 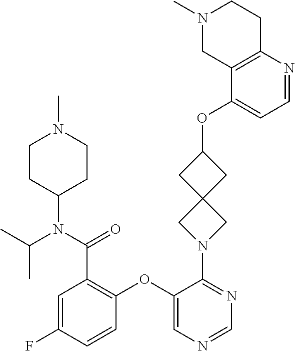 [SMILES: CC(C)N(C(=O)c1cc(F)ccc1Oc1cncnc1N1CC2(CC(Oc3ccnc4c3CN(C)CC4)C2)C1)C1CCN(C)CC1] | +++ | +++ | +++ |
| 49 | 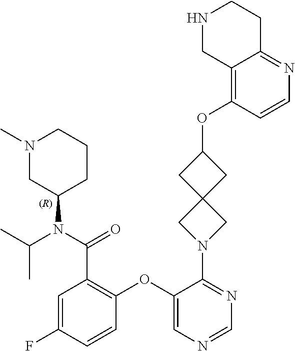 | +++ | +++ | +++ |
| 50 | 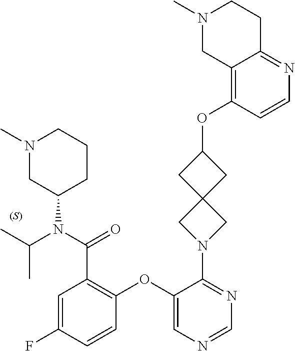 | +++ | +++ | +++ |
| 51 | 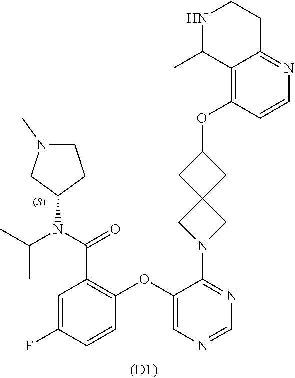 | +++ | +++ | +++ |
| 52 | 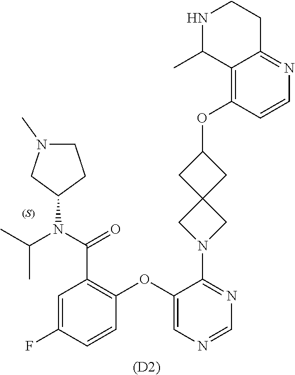 | +++ | +++ | +++ |
| 53 | 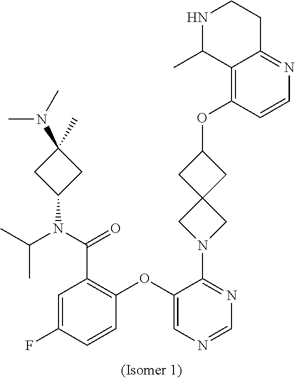 [SMILES: CC1NCCc2nccc(OC3CC4(C3)CN(c3ncncc3Oc3ccc(F)cc3C(=O)N(C(C)C)[C@H]3C[C@](C)(N(C)C)C3)C4)c21] | +++ | +++ | +++ |
| 54 | 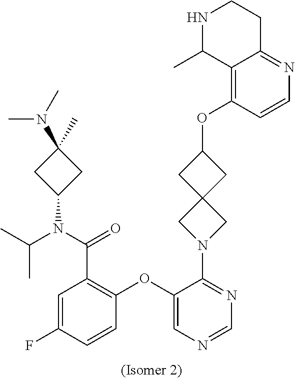 [SMILES: CC1NCCc2nccc(OC3CC4(C3)CN(c3ncncc3Oc3ccc(F)cc3C(=O)N(C(C)C)[C@H]3C[C@](C)(N(C)C)C3)C4)c21] | +++ | +++ | +++ |
| 55 | 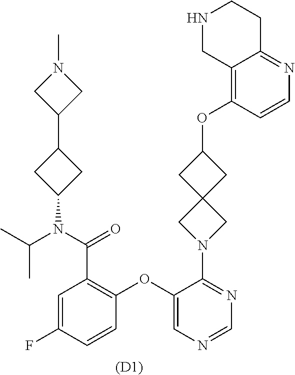 | +++ | +++ | +++ |
| 56 | 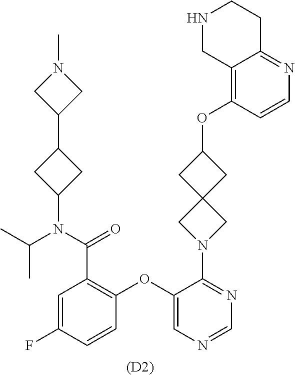 | +++ | +++ | +++ |
| 57 | 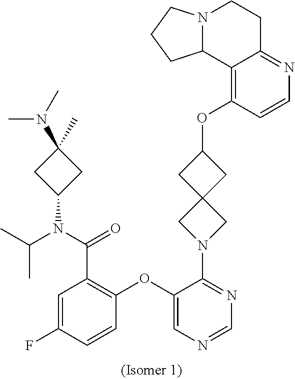 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(N(C)C)C1] | +++ | +++ | +++ |
| 58 | 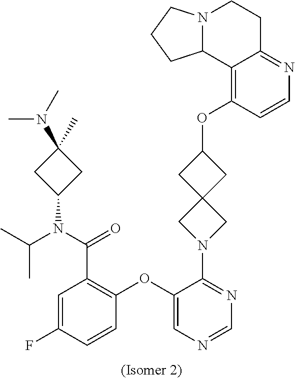 [SMILES: CC(C)N(C(=O)c1cc(F)ccc1Oc1cncnc1N1CC2(CC(Oc3ccnc4c3C3CCCN3CC4)C2)C1)[C@H]1C[C@](C)(N(C)C)C1] | +++ | +++ | +++ |
| 59 | 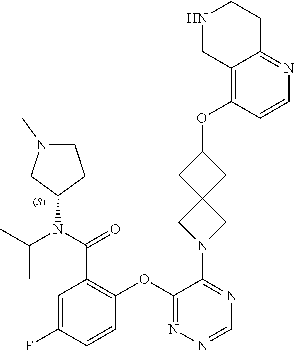 | +++ | +++ | +++ |
| 60 | 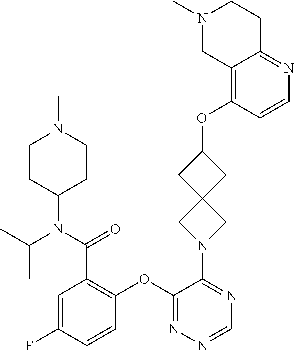 [SMILES: CC(C)N(C(=O)c1cc(F)ccc1Oc1nncnc1N1CC2(CC(Oc3ccnc4c3CN(C)CC4)C2)C1)C1CCN(C)CC1] | +++ | +++ | +++ |
| 61 | 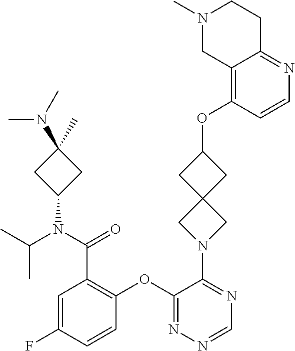 [SMILES: CC(C)N(C(=O)c1cc(F)ccc1Oc1nncnc1N1CC2(CC(Oc3ccnc4c3CN(C)CC4)C2)C1)[C@H]1C[C@](C)(N(C)C)C1] | +++ | +++ | +++ |
| 62 | 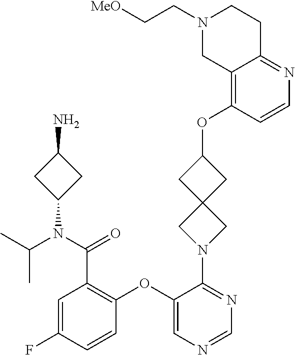 [SMILES: COCCN1CCc2nccc(OC3CC4(C3)CN(c3ncncc3Oc3ccc(F)cc3C(=O)N(C(C)C)[C@H]3C[C@H](N)C3)C4)c2C1] | +++ | +++ | +++ |
| 63 | 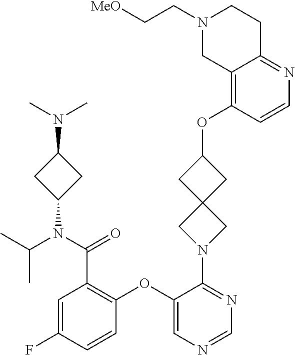 [SMILES: COCCN1CCc2nccc(OC3CC4(C3)CN(c3ncncc3Oc3ccc(F)cc3C(=O)N(C(C)C)[C@H]3C[C@H](N(C)C)C3)C4)c2C1] | +++ | +++ | +++ |
| 64 | 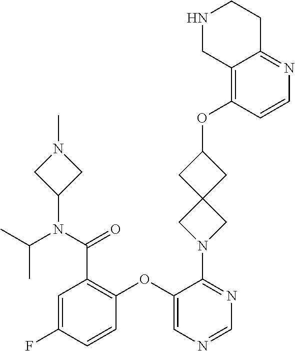 [SMILES: CC(C)N(C(=O)c1cc(F)ccc1Oc1cncnc1N1CC2(CC(Oc3ccnc4c3CNCC4)C2)C1)C1CN(C)C1] | +++ | +++ | +++ |
| 65 | 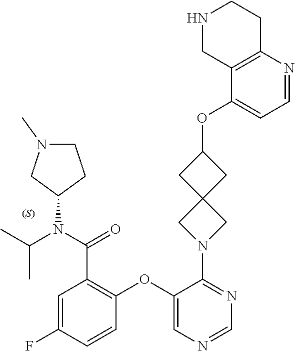 | +++ | +++ | +++ |
| 66 | 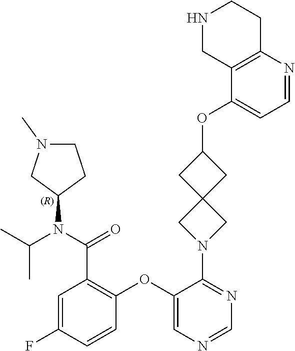 | +++ | +++ | +++ |
| 67 | 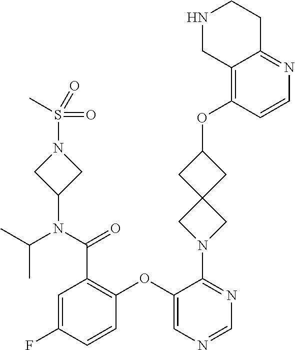 [SMILES: CC(C)N(C(=O)c1cc(F)ccc1Oc1cncnc1N1CC2(CC(Oc3ccnc4c3CNCC4)C2)C1)C1CN(S(C)(=O)=O)C1] | +++ | +++ | +++ |
| 68 | 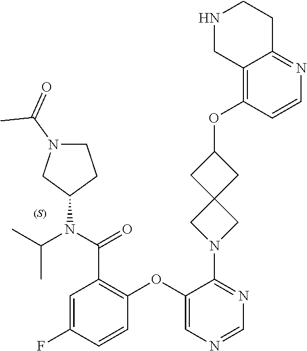 | +++ | +++ | ++ |
| 69 | 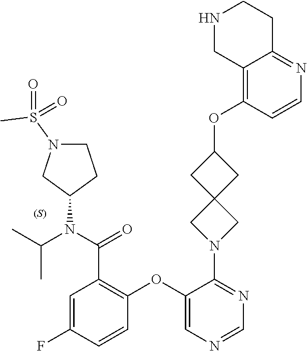 | +++ | +++ | +++ |
| 70 | 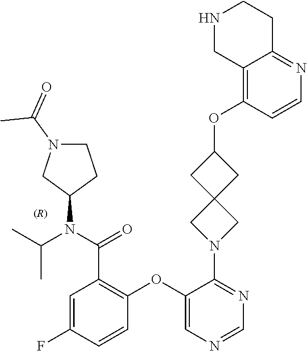 | +++ | ++ | ++ |
| 71 | 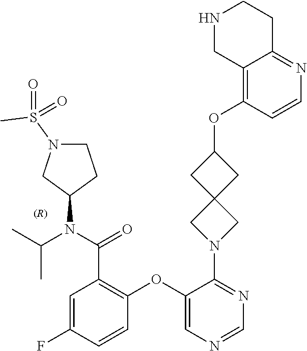 | +++ | +++ | ++ |
| 72 | 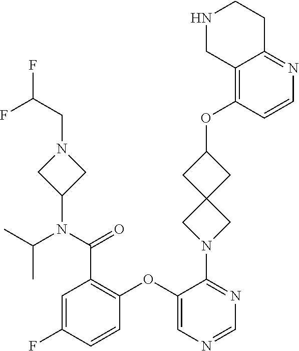 [SMILES: CC(C)N(C(=O)c1cc(F)ccc1Oc1cncnc1N1CC2(CC(Oc3ccnc4c3CNCC4)C2)C1)C1CN(CC(F)F)C1] | +++ | +++ | +++ |
| 73 | 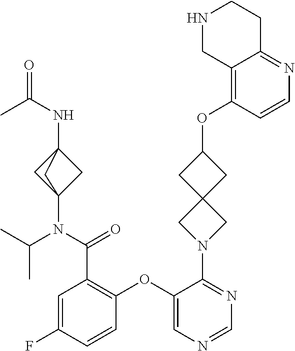 [SMILES: CC(=O)NC12CC(N(C(=O)c3cc(F)ccc3Oc3cncnc3N3CC4(CC(Oc5ccnc6c5CNCC6)C4)C3)C(C)C)(C1)C2] | +++ | +++ | ++ |
| 74 | 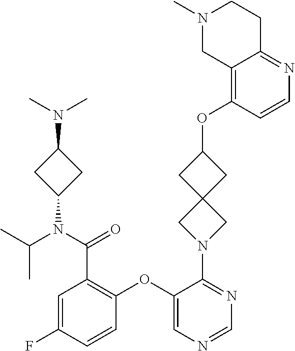 [SMILES: CC(C)N(C(=O)c1cc(F)ccc1Oc1cncnc1N1CC2(CC(Oc3ccnc4c3CN(C)CC4)C2)C1)[C@H]1C[C@H](N(C)C)C1] | +++ | +++ | +++ |
| 75 | 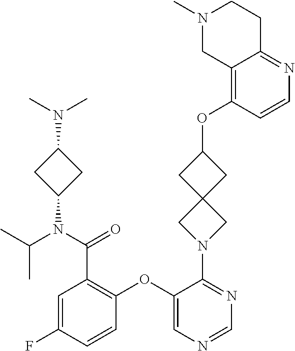 [SMILES: CC(C)N(C(=O)c1cc(F)ccc1Oc1cncnc1N1CC2(CC(Oc3ccnc4c3CN(C)CC4)C2)C1)[C@H]1C[C@@H](N(C)C)C1] | +++ | +++ | +++ |
| 76 | 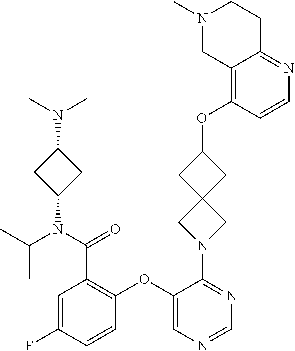 [SMILES: CC(C)N(C(=O)c1cc(F)ccc1Oc1cncnc1N1CC2(CC(Oc3ccnc4c3CN(C)CC4)C2)C1)[C@H]1C[C@@H](N(C)C)C1] | +++ | +++ | +++ |
| 77 | 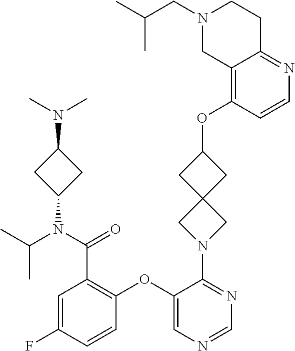 [SMILES: CC(C)CN1CCc2nccc(OC3CC4(C3)CN(c3ncncc3Oc3ccc(F)cc3C(=O)N(C(C)C)[C@H]3C[C@H](N(C)C)C3)C4)c2C1] | +++ | +++ | +++ |
| 78 | 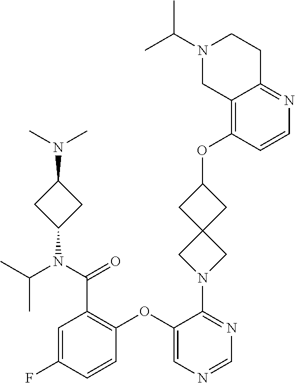 [SMILES: CC(C)N1CCc2nccc(OC3CC4(C3)CN(c3ncncc3Oc3ccc(F)cc3C(=O)N(C(C)C)[C@H]3C[C@H](N(C)C)C3)C4)c2C1] | +++ | +++ | +++ |
| 79 | 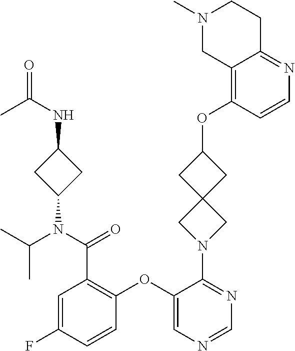 [SMILES: CC(=O)N[C@H]1C[C@H](N(C(=O)c2cc(F)ccc2Oc2cncnc2N2CC3(CC(Oc4ccnc5c4CN(C)CC5)C3)C2)C(C)C)C1] | +++ | +++ | +++ |
| 80 | 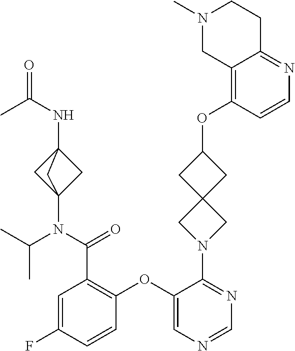 [SMILES: CC(=O)NC12CC(N(C(=O)c3cc(F)ccc3Oc3cncnc3N3CC4(CC(Oc5ccnc6c5CN(C)CC6)C4)C3)C(C)C)(C1)C2] | +++ | +++ | +++ |
| 81 | 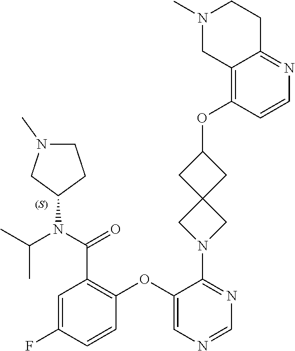 | +++ | +++ | +++ |
| 82 | 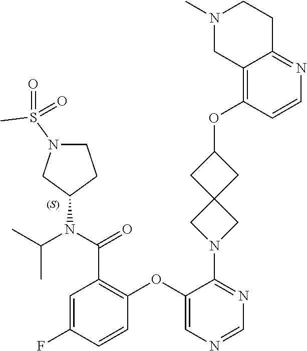 | +++ | +++ | +++ |
| 83 | 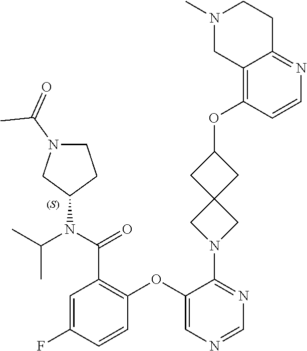 | +++ | +++ | +++ |
| 84 | 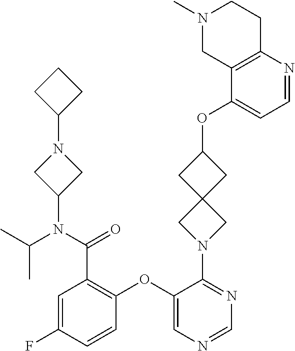 [SMILES: CC(C)N(C(=O)c1cc(F)ccc1Oc1cncnc1N1CC2(CC(Oc3ccnc4c3CN(C)CC4)C2)C1)C1CN(C2CCC2)C1] | +++ | +++ | +++ |
| 85 | 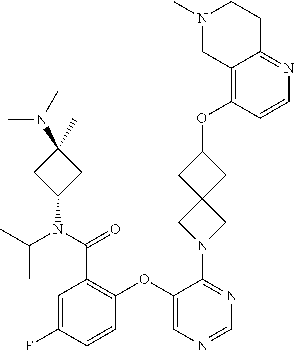 [SMILES: CC(C)N(C(=O)c1cc(F)ccc1Oc1cncnc1N1CC2(CC(Oc3ccnc4c3CN(C)CC4)C2)C1)[C@H]1C[C@](C)(N(C)C)C1] | +++ | +++ | +++ |
| 86 | 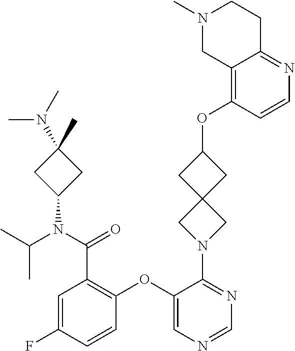 [SMILES: CC(C)N(C(=O)c1cc(F)ccc1Oc1cncnc1N1CC2(CC(Oc3ccnc4c3CN(C)CC4)C2)C1)[C@H]1C[C@@](C)(N(C)C)C1] | +++ | +++ | +++ |
| 87 | 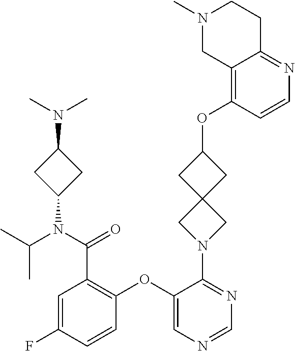 [SMILES: CC(C)N(C(=O)c1cc(F)ccc1Oc1cncnc1N1CC2(CC(Oc3ccnc4c3CN(C)CC4)C2)C1)[C@H]1C[C@H](N(C)C)C1] | +++ | +++ | +++ |
| 88 | 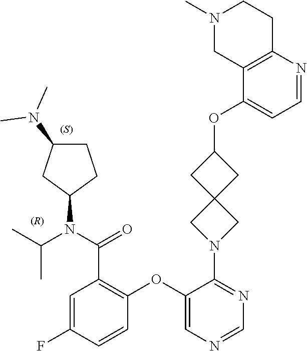 | +++ | +++ | +++ |
| 89 | 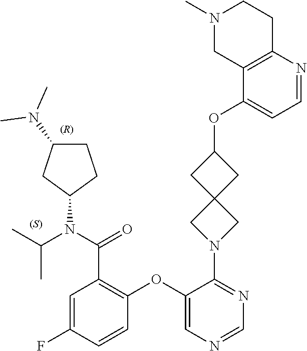 | +++ | +++ | +++ |
| 90 | 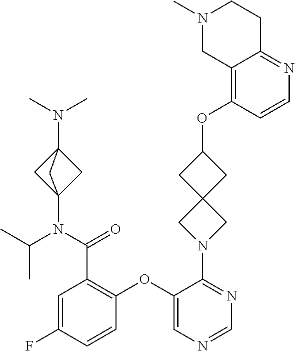 [SMILES: CC(C)N(C(=O)c1cc(F)ccc1Oc1cncnc1N1CC2(CC(Oc3ccnc4c3CN(C)CC4)C2)C1)C12CC(N(C)C)(C1)C2] | +++ | +++ | +++ |
| 91 | 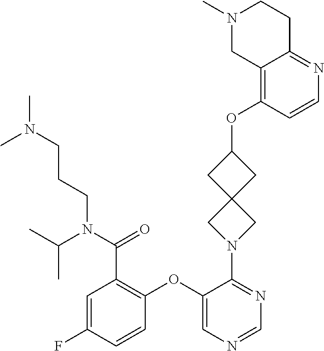 [SMILES: CC(C)N(CCCN(C)C)C(=O)c1cc(F)ccc1Oc1cncnc1N1CC2(CC(Oc3ccnc4c3CN(C)CC4)C2)C1] | +++ | +++ | +++ |
| 92 | 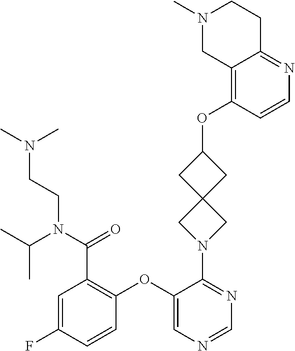 [SMILES: CC(C)N(CCN(C)C)C(=O)c1cc(F)ccc1Oc1cncnc1N1CC2(CC(Oc3ccnc4c3CN(C)CC4)C2)C1] | +++ | +++ | +++ |
| 93 | 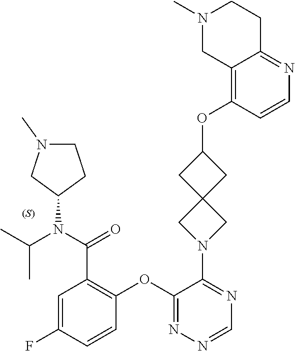 | +++ | +++ | +++ |
| 94 | 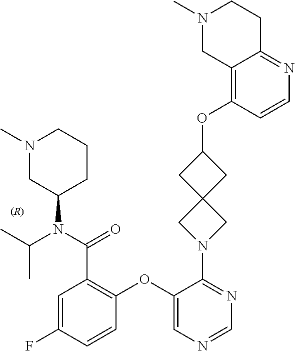 | +++ | +++ | +++ |
| 95 | 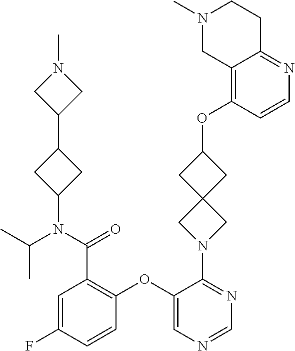 [SMILES: CC(C)N(C(=O)c1cc(F)ccc1Oc1cncnc1N1CC2(CC(Oc3ccnc4c3CN(C)CC4)C2)C1)C1CC(C2CN(C)C2)C1] | +++ | +++ | +++ |
| 96 | 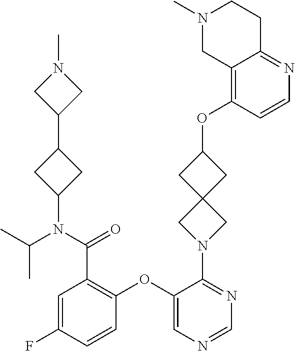 [SMILES: CC(C)N(C(=O)c1cc(F)ccc1Oc1cncnc1N1CC2(CC(Oc3ccnc4c3CN(C)CC4)C2)C1)C1CC(C2CN(C)C2)C1] | +++ | +++ | +++ |
| 97 | 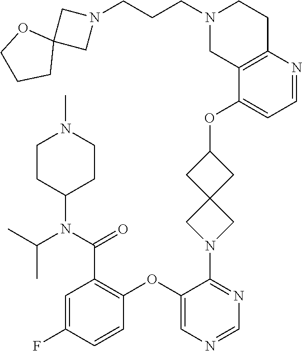 [SMILES: CC(C)N(C(=O)c1cc(F)ccc1Oc1cncnc1N1CC2(CC(Oc3ccnc4c3CN(CCCN3CC5(CCCO5)C3)CC4)C2)C1)C1CCN(C)CC1] | +++ | +++ | +++ |
| 98 | 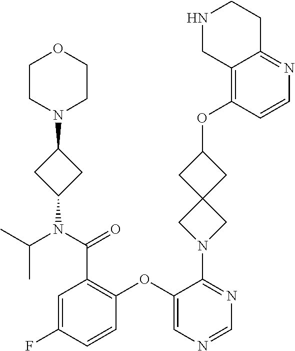 [SMILES: CC(C)N(C(=O)c1cc(F)ccc1Oc1cncnc1N1CC2(CC(Oc3ccnc4c3CNCC4)C2)C1)[C@H]1C[C@H](N2CCOCC2)C1] | +++ | +++ | +++ |
| 99 | 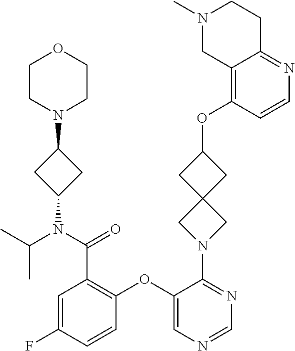 [SMILES: CC(C)N(C(=O)c1cc(F)ccc1Oc1cncnc1N1CC2(CC(Oc3ccnc4c3CN(C)CC4)C2)C1)[C@H]1C[C@H](N2CCOCC2)C1] | +++ | +++ | +++ |
| 100 | 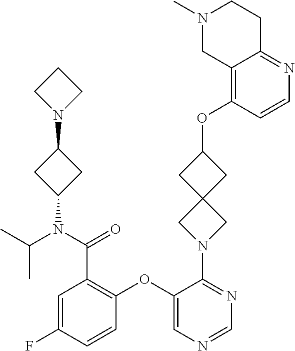 [SMILES: CC(C)N(C(=O)c1cc(F)ccc1Oc1cncnc1N1CC2(CC(Oc3ccnc4c3CN(C)CC4)C2)C1)[C@H]1C[C@H](N2CCC2)C1] | +++ | +++ | +++ |
| 101 | 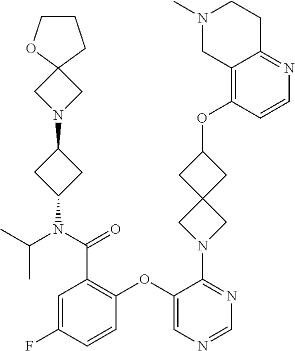 [SMILES: CC(C)N(C(=O)c1cc(F)ccc1Oc1cncnc1N1CC2(CC(Oc3ccnc4c3CN(C)CC4)C2)C1)[C@H]1C[C@H](N2CC3(CCCO3)C2)C1] | +++ | +++ | +++ |
| 102 | 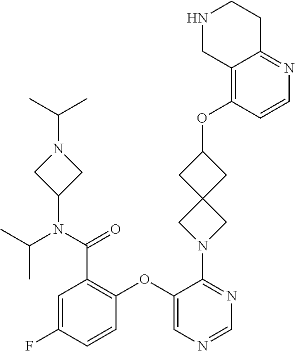 [SMILES: CC(C)N1CC(N(C(=O)c2cc(F)ccc2Oc2cncnc2N2CC3(CC(Oc4ccnc5c4CNCC5)C3)C2)C(C)C)C1] | +++ | +++ | +++ |
| 103 | 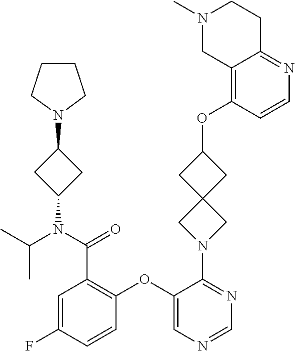 [SMILES: CC(C)N(C(=O)c1cc(F)ccc1Oc1cncnc1N1CC2(CC(Oc3ccnc4c3CN(C)CC4)C2)C1)[C@H]1C[C@H](N2CCCC2)C1] | +++ | +++ | +++ |
| 104 | 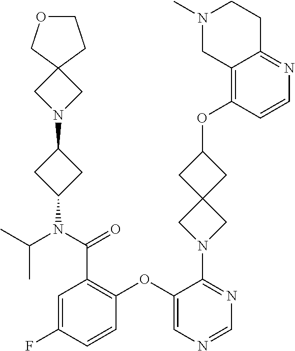 [SMILES: CC(C)N(C(=O)c1cc(F)ccc1Oc1cncnc1N1CC2(CC(Oc3ccnc4c3CN(C)CC4)C2)C1)[C@H]1C[C@H](N2CC3(CCOC3)C2)C1] | +++ | +++ | +++ |
| 105 | 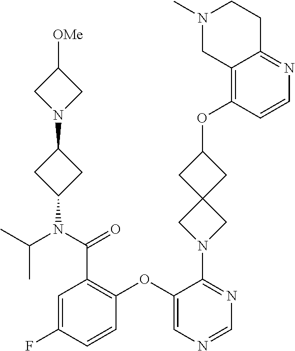 [SMILES: COC1CN([C@H]2C[C@H](N(C(=O)c3cc(F)ccc3Oc3cncnc3N3CC4(CC(Oc5ccnc6c5CN(C)CC6)C4)C3)C(C)C)C2)C1] | +++ | +++ | +++ |
| 106 | 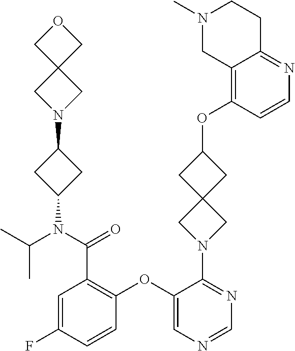 [SMILES: CC(C)N(C(=O)c1cc(F)ccc1Oc1cncnc1N1CC2(CC(Oc3ccnc4c3CN(C)CC4)C2)C1)[C@H]1C[C@H](N2CC3(COC3)C2)C1] | +++ | +++ | +++ |
| 107 | 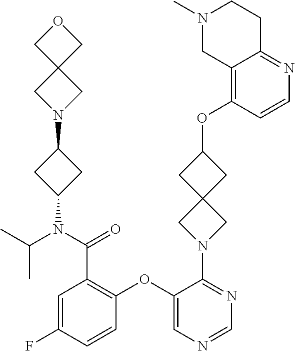 [SMILES: CC(C)N(C(=O)c1cc(F)ccc1Oc1cncnc1N1CC2(CC(Oc3ccnc4c3CN(C)CC4)C2)C1)[C@H]1C[C@H](N2CC3(COC3)C2)C1] | +++ | +++ | +++ |
| 108 | 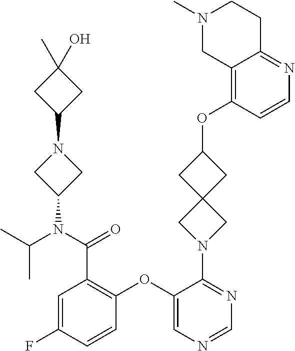 | +++ | +++ | +++ |
| 109 | 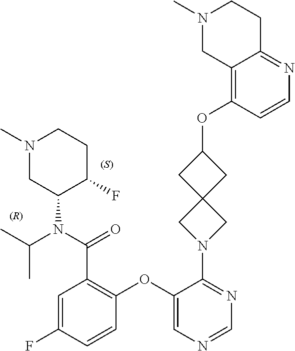 | +++ | +++ | +++ |
| 110 | 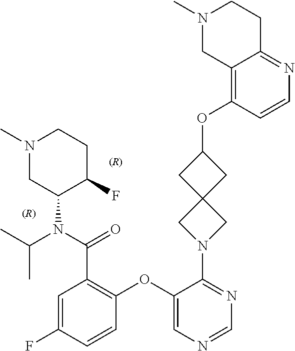 | +++ | +++ | +++ |
| 111 | 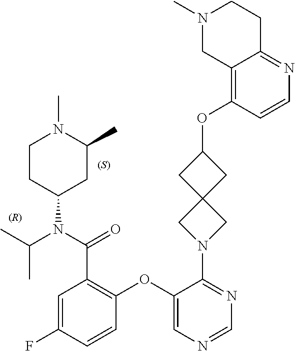 | +++ | +++ | +++ |
| 112 | 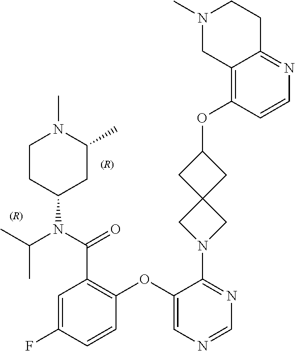 | +++ | +++ | +++ |
| 113 | 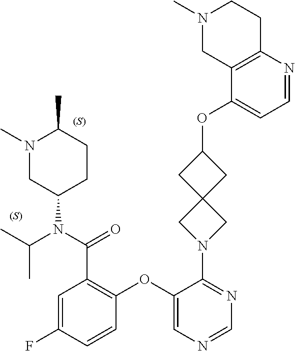 | +++ | +++ | +++ |
| 114 | 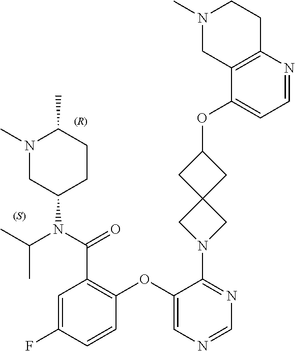 | +++ | +++ | +++ |
| 115 | 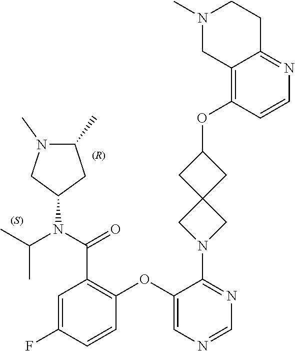 | +++ | +++ | +++ |
| 116 | 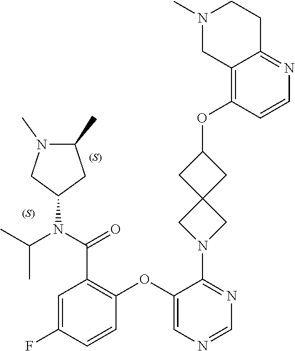 | +++ | +++ | +++ |
| 117 | 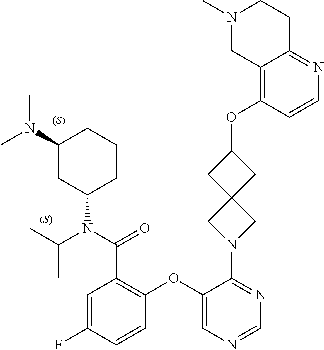 | +++ | +++ | +++ |
| 118 | 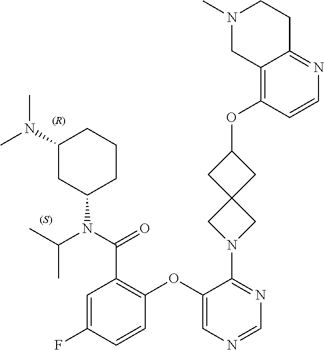 | +++ | +++ | +++ |
| 119 | 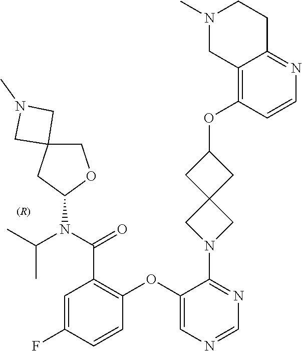 | +++ | +++ | +++ |
| 120 | 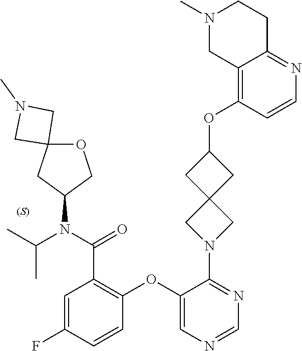 | +++ | +++ | +++ |
| 121 | 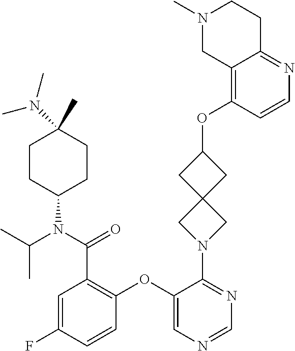 [SMILES: CC(C)N(C(=O)c1cc(F)ccc1Oc1cncnc1N1CC2(CC(Oc3ccnc4c3CN(C)CC4)C2)C1)[C@H]1CC[C@@](C)(N(C)C)CC1] | +++ | +++ | +++ |
| 122 | 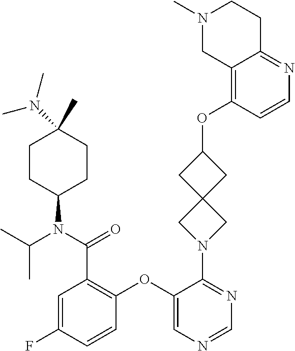 [SMILES: CC(C)N(C(=O)c1cc(F)ccc1Oc1cncnc1N1CC2(CC(Oc3ccnc4c3CN(C)CC4)C2)C1)[C@H]1CC[C@](C)(N(C)C)CC1] | +++ | +++ | +++ |
| 123 | 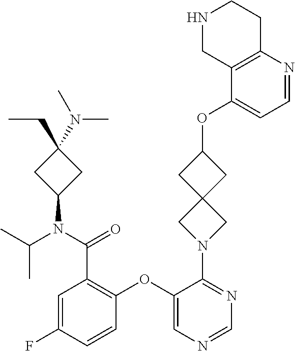 [SMILES: CC[C@]1(N(C)C)C[C@H](N(C(=O)c2cc(F)ccc2Oc2cncnc2N2CC3(CC(Oc4ccnc5c4CNCC5)C3)C2)C(C)C)C1] | +++ | +++ | +++ |
| 124 | 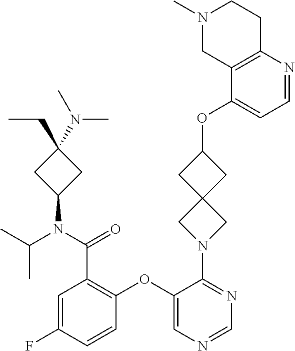 [SMILES: CC[C@]1(N(C)C)C[C@H](N(C(=O)c2cc(F)ccc2Oc2cncnc2N2CC3(CC(Oc4ccnc5c4CN(C)CC5)C3)C2)C(C)C)C1] | +++ | +++ | +++ |
| 125 | 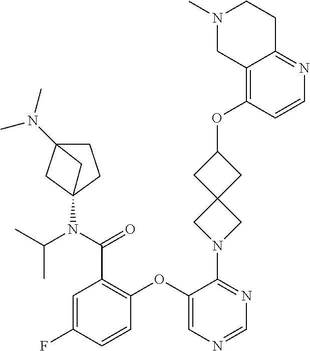 | +++ | +++ | +++ |
| 126 | 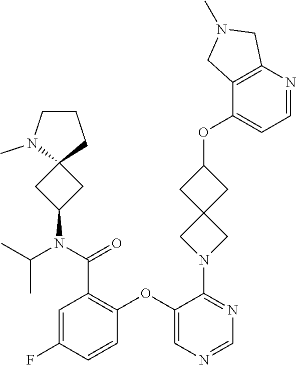 [SMILES: CC(C)N(C(=O)c1cc(F)ccc1Oc1cncnc1N1CC2(CC(Oc3ccnc4c3CN(C)C4)C2)C1)[C@H]1C[C@@]2(CCCN2C)C1] | +++ | +++ | +++ |
| 127 | 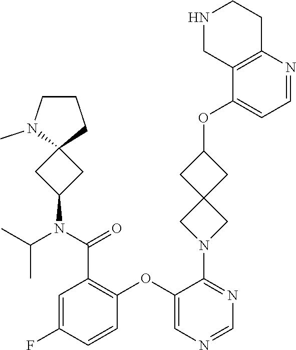 [SMILES: CC(C)N(C(=O)c1cc(F)ccc1Oc1cncnc1N1CC2(CC(Oc3ccnc4c3CNCC4)C2)C1)[C@H]1C[C@@]2(CCCN2C)C1] | +++ | +++ | +++ |
| 128 | 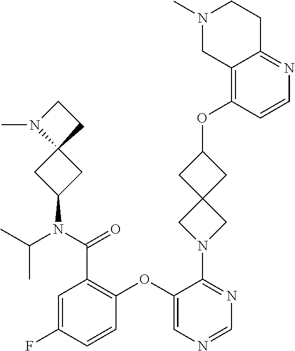 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCN2C)C1] | +++ | +++ | +++ |
| 129 | 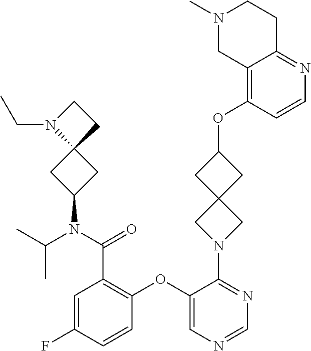 [SMILES: CCN1CC[C@]12C[C@@H](N(C(=O)c1cc(F)ccc1Oc1cncnc1N1CC3(CC(Oc4ccnc5c4CN(C)CC5)C3)C1)C(C)C)C2] | +++ | +++ | +++ |
| 130 | 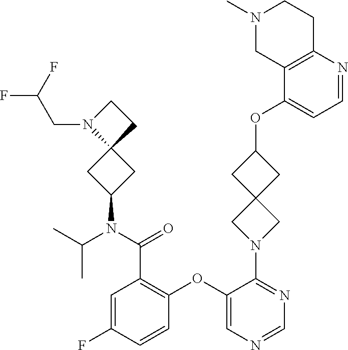 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCN2CC(F)F)C1] | +++ | +++ | +++ |
| 131 | 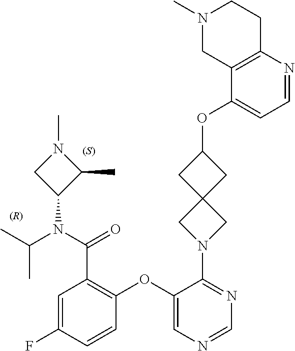 | +++ | +++ | +++ |
| 132 | 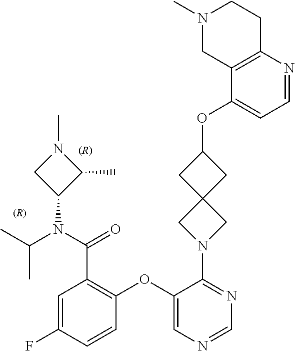 | +++ | +++ | +++ |
| 133 | 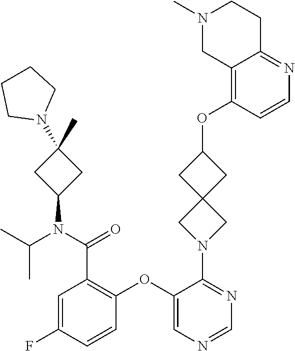 [SMILES: CC(C)N(C(=O)c1cc(F)ccc1Oc1cncnc1N1CC2(CC(Oc3ccnc4c3CN(C)CC4)C2)C1)[C@H]1C[C@](C)(N2CCCC2)C1] | +++ | +++ | +++ |
| 134 | 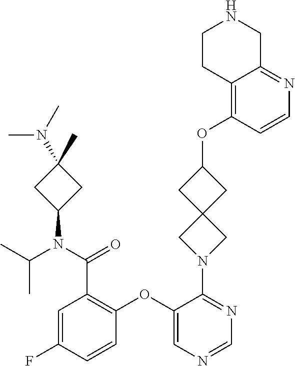 [SMILES: CC(C)N(C(=O)c1cc(F)ccc1Oc1cncnc1N1CC2(CC(Oc3ccnc4c3CCNC4)C2)C1)[C@H]1C[C@](C)(N(C)C)C1] | +++ | +++ | +++ |
| 135 | 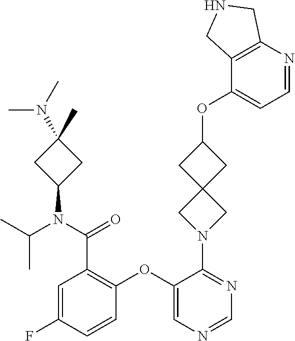 [SMILES: CC(C)N(C(=O)c1cc(F)ccc1Oc1cncnc1N1CC2(CC(Oc3ccnc4c3CNC4)C2)C1)[C@H]1C[C@](C)(N(C)C)C1] | +++ | +++ | +++ |
| 136 | 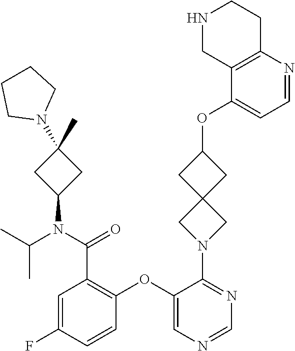 [SMILES: CC(C)N(C(=O)c1cc(F)ccc1Oc1cncnc1N1CC2(CC(Oc3ccnc4c3CNCC4)C2)C1)[C@H]1C[C@](C)(N2CCCC2)C1] | +++ | +++ | +++ |
| 137 | 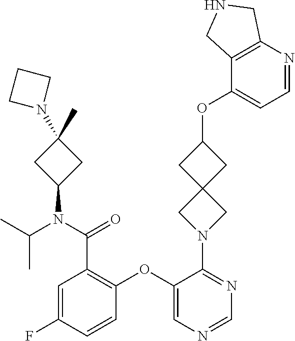 [SMILES: CC(C)N(C(=O)c1cc(F)ccc1Oc1cncnc1N1CC2(CC(Oc3ccnc4c3CNC4)C2)C1)[C@H]1C[C@](C)(N2CCC2)C1] | +++ | +++ | +++ |
| 138 | 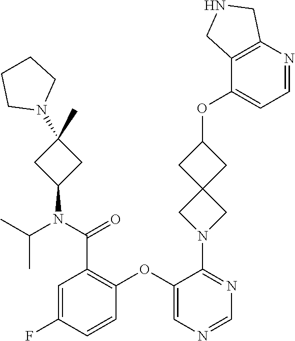 [SMILES: CC(C)N(C(=O)c1cc(F)ccc1Oc1cncnc1N1CC2(CC(Oc3ccnc4c3CNC4)C2)C1)[C@H]1C[C@](C)(N2CCCC2)C1] | +++ | +++ | +++ |
| 139 | 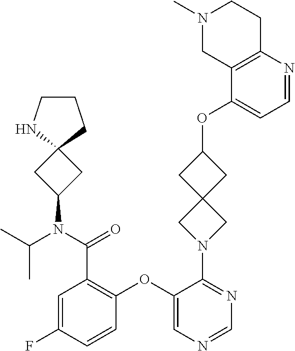 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCN2)C1] | +++ | +++ | +++ |
| 140 | 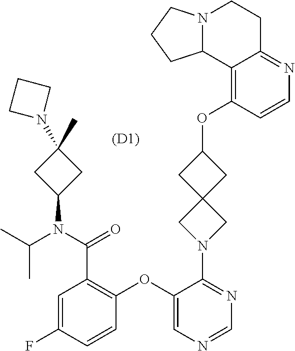 | +++ | +++ | +++ |
| 141 | 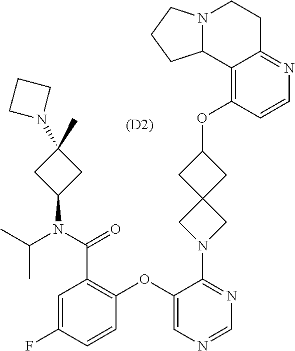 | +++ | +++ | +++ |
| 142 | 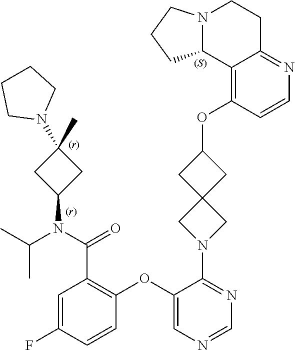 | +++ | +++ | +++ |
| 143 | 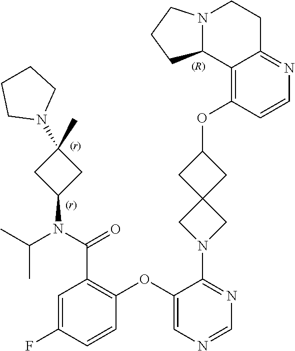 | +++ | +++ | +++ |
| 144 | 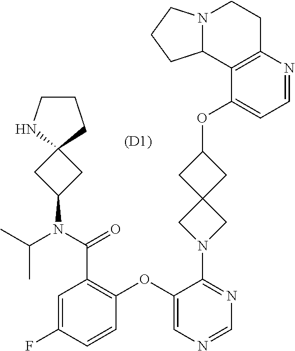 | +++ | +++ | ++ |
| 145 | 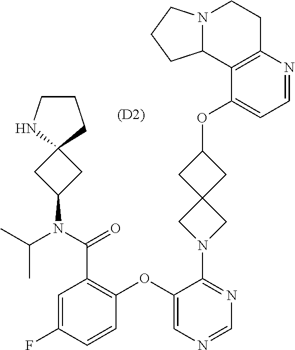 | +++ | +++ | +++ |
| 146 | 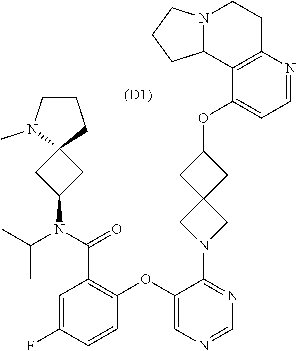 [SMILES: CC(C)N(C(=O)c1cc(F)ccc1Oc1cncnc1N1CC2(CC(Oc3ccnc4c3C3CCCN3CC4)C2)C1)[C@H]1C[C@@]2(CCCN2C)C1 ; "(D1)"] | +++ | +++ | +++ |
| 147 | 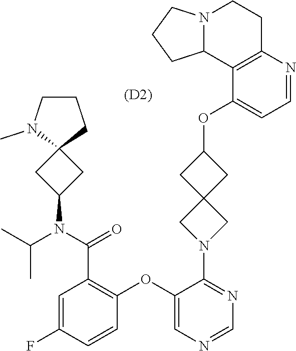 | +++ | +++ | +++ |
| 148 | 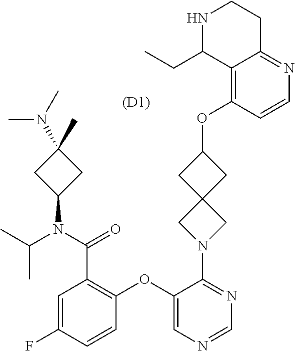 [SMILES: CCC1NCCc2nccc(OC3CC4(C3)CN(c3ncncc3Oc3ccc(F)cc3C(=O)N(C(C)C)[C@H]3C[C@](C)(N(C)C)C3)C4)c21 ; "(D1)"] | +++ | +++ | +++ |
| 149 | 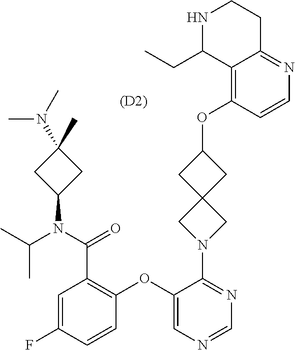 | +++ | +++ | +++ |
| 150 | 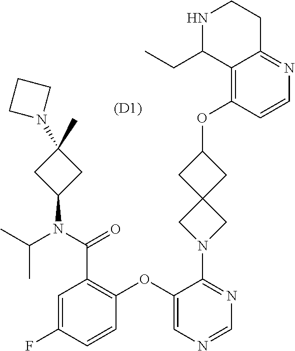 | +++ | +++ | +++ |
| 151 | 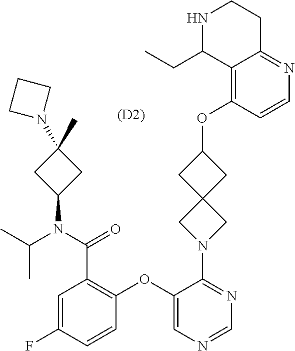 | +++ | +++ | +++ |
| 152 | 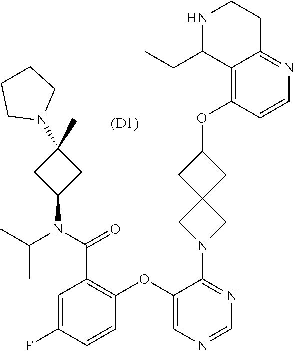 | +++ | +++ | +++ |
| 153 | 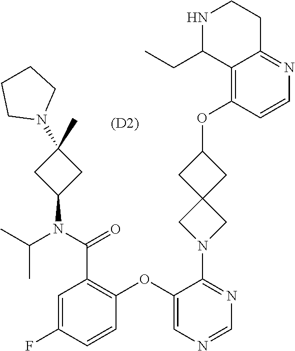 | +++ | +++ | +++ |
| 154 | 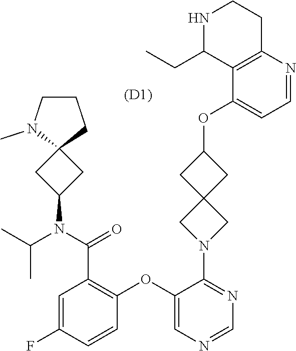 [SMILES: CCC1NCCc2nccc(OC3CC4(C3)CN(c3ncncc3Oc3ccc(F)cc3C(=O)N(C(C)C)[C@H]3C[C@@]5(CCCN5C)C3)C4)c21 ; "(D1)"] | +++ | +++ | +++ |
| 155 | 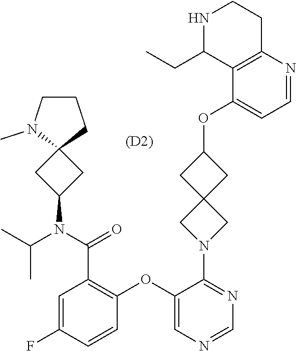 | +++ | +++ | +++ |
| 156 | 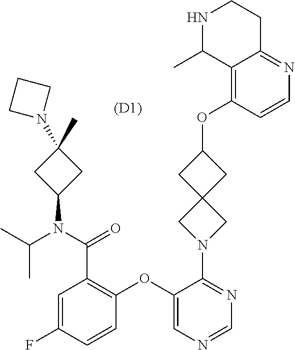 | +++ | +++ | +++ |
| 157 | 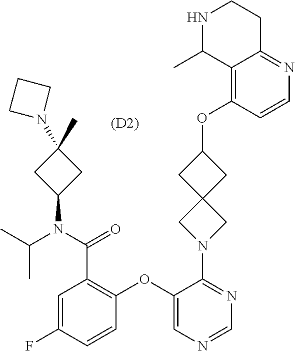 | +++ | +++ | +++ |
| 158 | 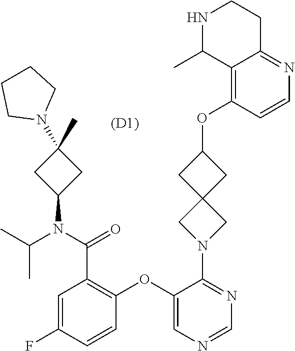 | +++ | +++ | +++ |
| 159 | 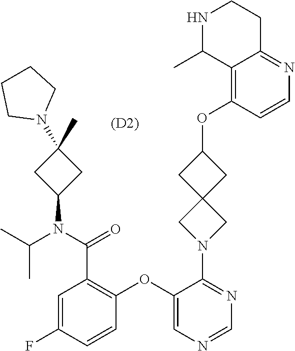 | +++ | +++ | +++ |
| 160 | 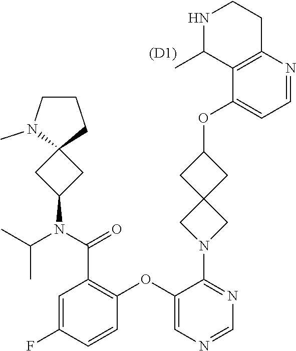 | +++ | +++ | +++ |
| 161 | 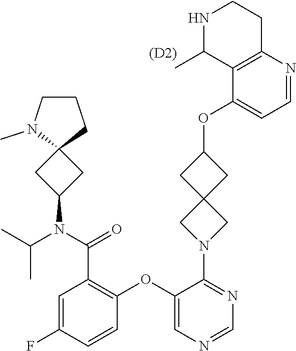 | +++ | +++ | +++ |
| 162 | 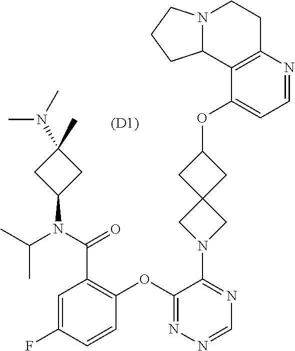 | +++ | +++ | +++ |
| 163 | 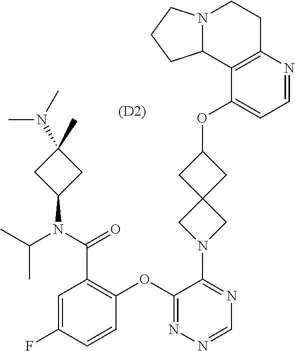 | +++ | +++ | +++ |
| 164 | 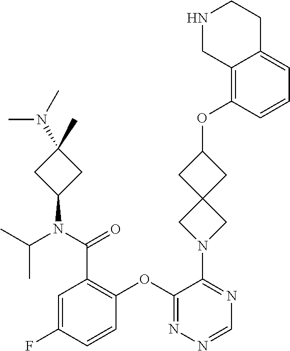 [SMILES: CC(C)N(C(=O)c1cc(F)ccc1Oc1nncnc1N1CC2(CC(Oc3cccc4c3CNCC4)C2)C1)[C@H]1C[C@](C)(N(C)C)C1] | +++ | +++ | +++ |
| 165 | 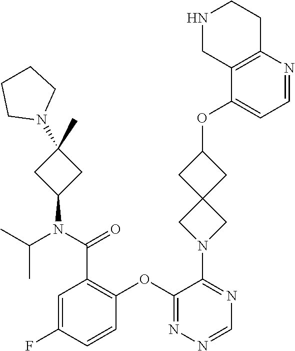 [SMILES: CC(C)N(C(=O)c1cc(F)ccc1Oc1nncnc1N1CC2(CC(Oc3ccnc4c3CNCC4)C2)C1)[C@H]1C[C@](C)(N2CCCC2)C1] | +++ | +++ | +++ |
| 166 | 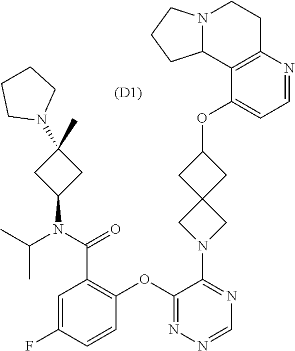 [SMILES: CC(C)N(C(=O)c1cc(F)ccc1Oc1nncnc1N1CC2(CC(Oc3ccnc4c3C3CCCN3CC4)C2)C1)[C@H]1C[C@](C)(N2CCCC2)C1 ; "(D1)"] | +++ | +++ | +++ |
| 167 | 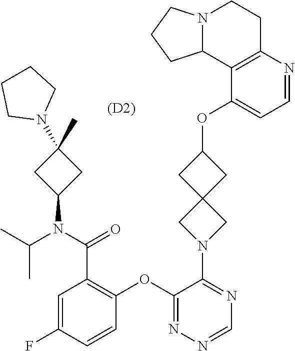 | +++ | +++ | +++ |
| 168 | 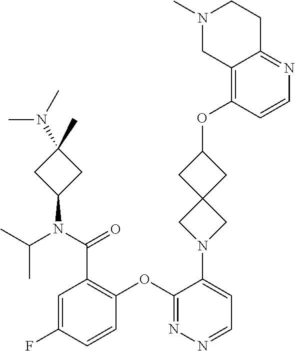 [SMILES: CC(C)N(C(=O)c1cc(F)ccc1Oc1nnccc1N1CC2(CC(Oc3ccnc4c3CN(C)CC4)C2)C1)[C@H]1C[C@](C)(N(C)C)C1] | +++ | +++ | +++ |
| 169 | 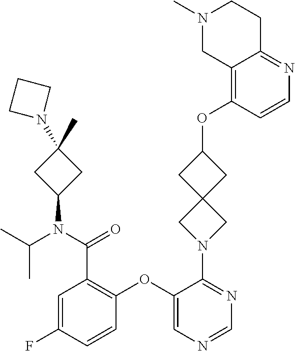 [SMILES: CC(C)N(C(=O)c1cc(F)ccc1Oc1cncnc1N1CC2(CC(Oc3ccnc4c3CN(C)CC4)C2)C1)[C@H]1C[C@](C)(N2CCC2)C1] | +++ | +++ | +++ |
| 170 | 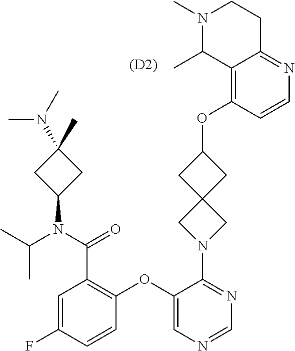 [SMILES: CC1c2c(OC3CC4(C3)CN(c3ncncc3Oc3ccc(F)cc3C(=O)N(C(C)C)[C@H]3C[C@](C)(N(C)C)C3)C4)ccnc2CCN1C ; "(D2)"] | +++ | +++ | +++ |
| 171 | 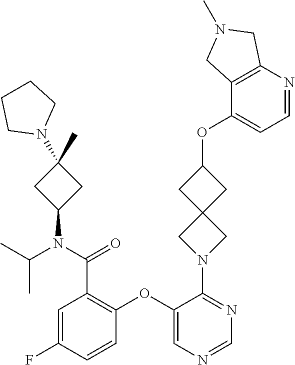 [SMILES: CC(C)N(C(=O)c1cc(F)ccc1Oc1cncnc1N1CC2(CC(Oc3ccnc4c3CN(C)C4)C2)C1)[C@H]1C[C@](C)(N2CCCC2)C1] | +++ | +++ | +++ |
| 172 | 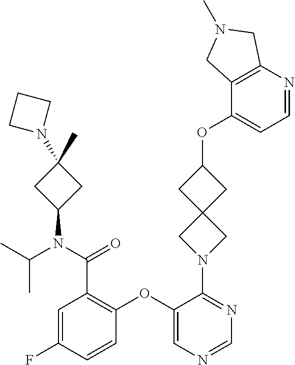 [SMILES: CC(C)N(C(=O)c1cc(F)ccc1Oc1cncnc1N1CC2(CC(Oc3ccnc4c3CN(C)C4)C2)C1)[C@H]1C[C@](C)(N2CCC2)C1] | +++ | +++ | +++ |
| 173 | 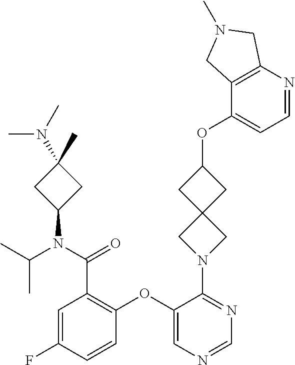 [SMILES: CC(C)N(C(=O)c1cc(F)ccc1Oc1cncnc1N1CC2(CC(Oc3ccnc4c3CN(C)C4)C2)C1)[C@H]1C[C@](C)(N(C)C)C1] | +++ | +++ | +++ |
| 174 | 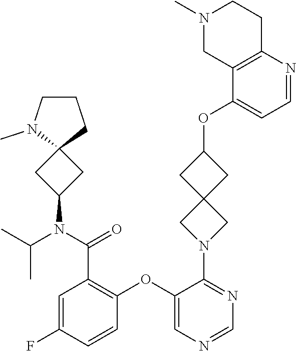 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCN2C)C1] | +++ | +++ | +++ |
| 175 | 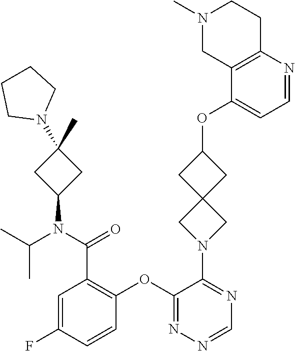 [SMILES: CC(C)N(C(=O)c1cc(F)ccc1Oc1nncnc1N1CC2(CC(Oc3ccnc4c3CN(C)CC4)C2)C1)[C@H]1C[C@](C)(N2CCCC2)C1] | +++ | +++ | +++ |
| 176 | 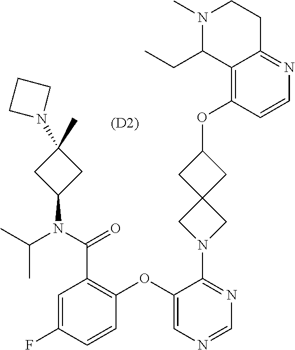 | +++ | +++ | +++ |
| 177 | 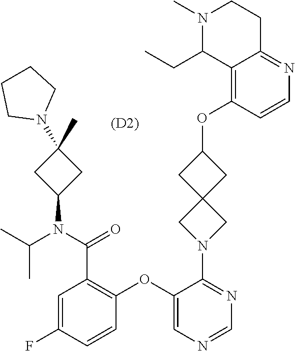 | +++ | +++ | +++ |
| 178 | 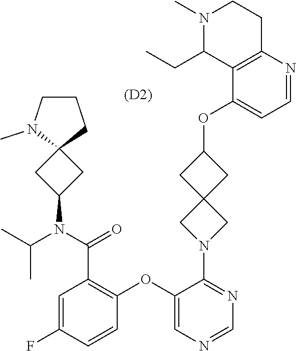 | +++ | +++ | +++ |
| 179 | 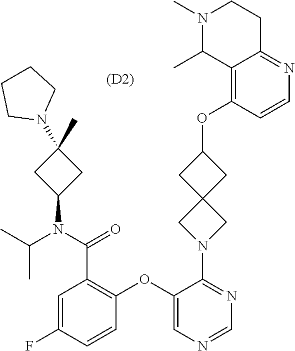 | +++ | +++ | +++ |
| 180 | 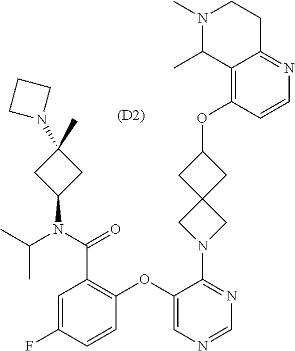 | +++ | +++ | +++ |
| 181 | 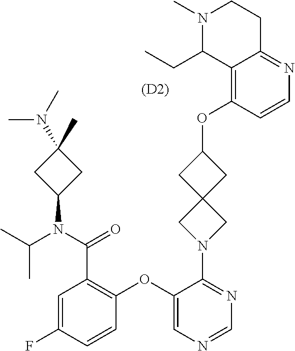 | +++ | +++ | +++ |
| 182 | 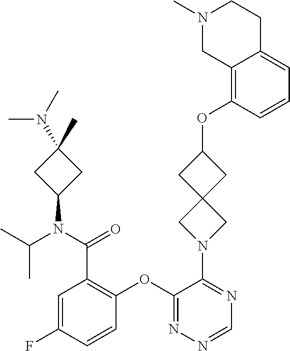 [SMILES: CC(C)N(C(=O)c1cc(F)ccc1Oc1nncnc1N1CC2(CC(Oc3cccc4c3CN(C)CC4)C2)C1)[C@H]1C[C@](C)(N(C)C)C1] | +++ | +++ | +++ |
| 183 | 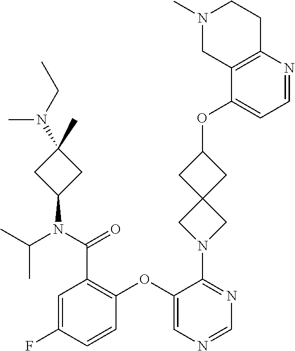 [SMILES: CCN(C)[C@]1(C)C[C@@H](N(C(=O)c2cc(F)ccc2Oc2cncnc2N2CC3(CC(Oc4ccnc5c4CN(C)CC5)C3)C2)C(C)C)C1] | +++ | +++ | +++ |
| 184 | 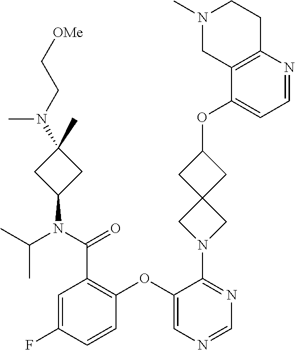 [SMILES: COCCN(C)[C@]1(C)C[C@@H](N(C(=O)c2cc(F)ccc2Oc2cncnc2N2CC3(CC(Oc4ccnc5c4CN(C)CC5)C3)C2)C(C)C)C1] | +++ | +++ | +++ |
| 185 | 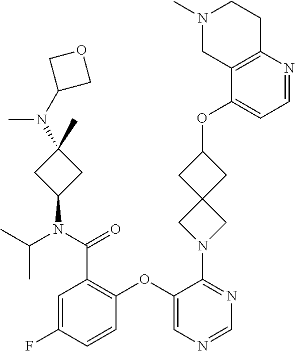 [SMILES: CC(C)N(C(=O)c1cc(F)ccc1Oc1cncnc1N1CC2(CC(Oc3ccnc4c3CN(C)CC4)C2)C1)[C@H]1C[C@](C)(N(C)C2COC2)C1] | +++ | +++ | +++ |
| 186 | 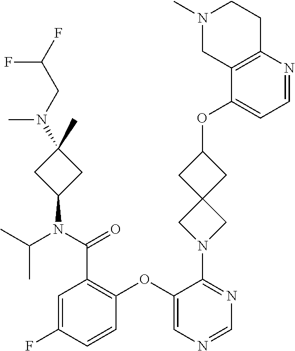 [SMILES: CC(C)N(C(=O)c1cc(F)ccc1Oc1cncnc1N1CC2(CC(Oc3ccnc4c3CN(C)CC4)C2)C1)[C@H]1C[C@](C)(N(C)CC(F)F)C1] | +++ | +++ | +++ |
| 187 | 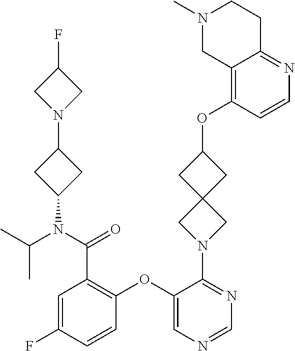 | +++ | +++ | +++ |
| 188 | 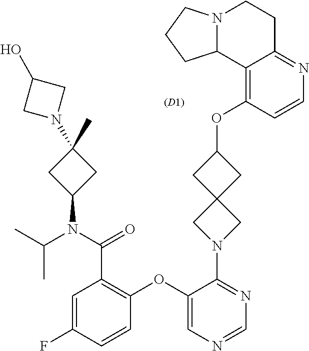 | +++ | +++ | +++ |
| 189 | 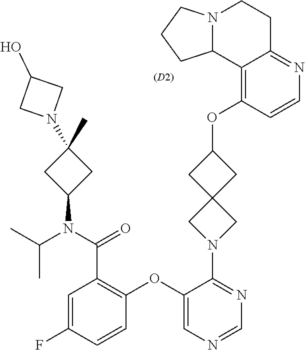 | +++ | +++ | +++ |
| 190 | 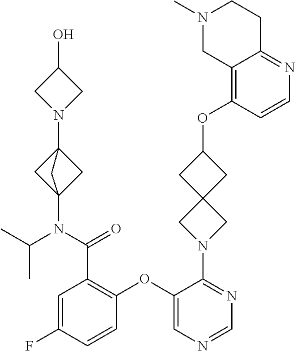 [SMILES: CC(C)N(C(=O)c1cc(F)ccc1Oc1cncnc1N1CC2(CC(Oc3ccnc4c3CN(C)CC4)C2)C1)C12CC(N3CC(O)C3)(C1)C2] | +++ | +++ | +++ |
| 191 | 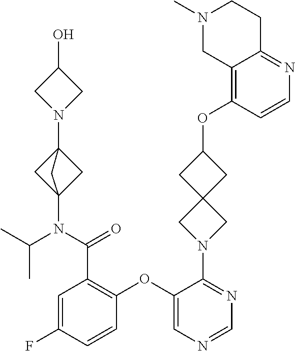 [SMILES: CC(C)N(C(=O)c1cc(F)ccc1Oc1cncnc1N1CC2(CC(Oc3ccnc4c3CN(C)CC4)C2)C1)C12CC(N3CC(O)C3)(C1)C2] | +++ | +++ | +++ |
| 192 | 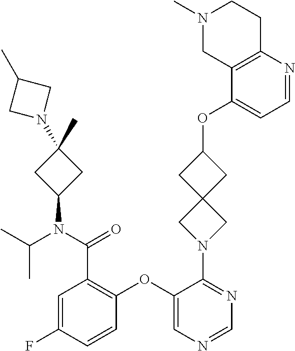 [SMILES: CC1CN([C@]2(C)C[C@@H](N(C(=O)c3cc(F)ccc3Oc3cncnc3N3CC4(CC(Oc5ccnc6c5CN(C)CC6)C4)C3)C(C)C)C2)C1] | +++ | +++ | +++ |
| 193 | 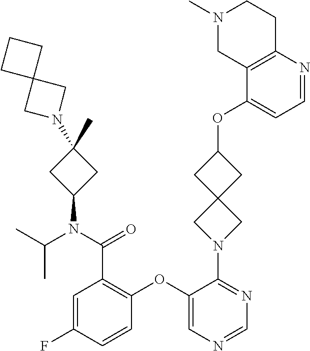 [SMILES: CC(C)N(C(=O)c1cc(F)ccc1Oc1cncnc1N1CC2(CC(Oc3ccnc4c3CN(C)CC4)C2)C1)[C@H]1C[C@](C)(N2CC3(CCC3)C2)C1] | +++ | +++ | +++ |
| 194 | 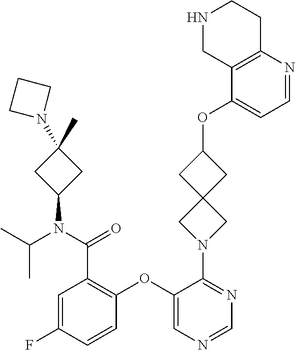 [SMILES: CC(C)N(C(=O)c1cc(F)ccc1Oc1cncnc1N1CC2(CC(Oc3ccnc4c3CNCC4)C2)C1)[C@H]1C[C@](C)(N2CCC2)C1] | +++ | +++ | +++ |
| 195 | 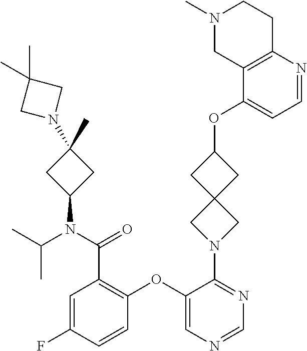 [SMILES: CC(C)N(C(=O)c1cc(F)ccc1Oc1cncnc1N1CC2(CC(Oc3ccnc4c3CN(C)CC4)C2)C1)[C@H]1C[C@](C)(N2CC(C)(C)C2)C1] | +++ | +++ | +++ |
| 196 | 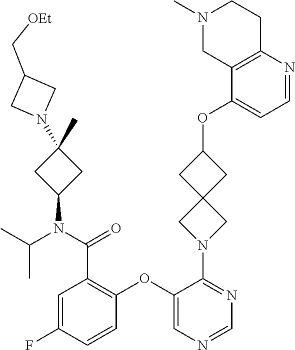 [SMILES: CCOCC1CN([C@]2(C)C[C@@H](N(C(=O)c3cc(F)ccc3Oc3cncnc3N3CC4(CC(Oc5ccnc6c5CN(C)CC6)C4)C3)C(C)C)C2)C1] | +++ | +++ | +++ |
| 197 | 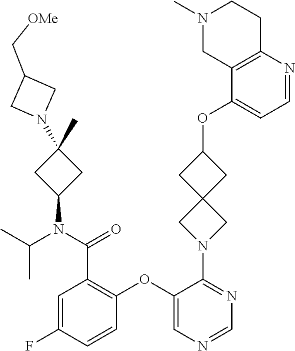 [SMILES: COCC1CN([C@]2(C)C[C@@H](N(C(=O)c3cc(F)ccc3Oc3cncnc3N3CC4(CC(Oc5ccnc6c5CN(C)CC6)C4)C3)C(C)C)C2)C1] | +++ | +++ | +++ |
| 198 | 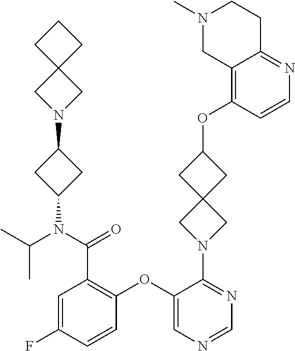 [SMILES: CC(C)N(C(=O)c1cc(F)ccc1Oc1cncnc1N1CC2(CC(Oc3ccnc4c3CN(C)CC4)C2)C1)[C@H]1C[C@H](N2CC3(CCC3)C2)C1] | +++ | +++ | +++ |
| 199 | 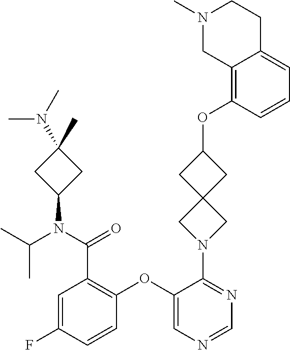 [SMILES: CC(C)N(C(=O)c1cc(F)ccc1Oc1cncnc1N1CC2(CC(Oc3cccc4c3CN(C)CC4)C2)C1)[C@H]1C[C@](C)(N(C)C)C1] | +++ | +++ | +++ |
| 200 | 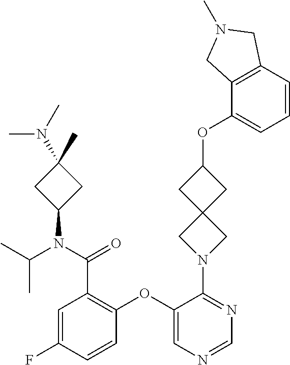 [SMILES: CC(C)N(C(=O)c1cc(F)ccc1Oc1cncnc1N1CC2(CC(Oc3cccc4c3CN(C)C4)C2)C1)[C@H]1C[C@](C)(N(C)C)C1] | +++ | +++ | +++ |
| 201 | 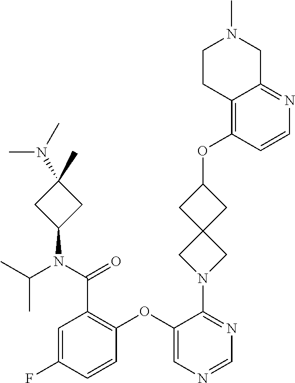 [SMILES: CC(C)N(C(=O)c1cc(F)ccc1Oc1cncnc1N1CC2(CC(Oc3ccnc4c3CCN(C)C4)C2)C1)[C@H]1C[C@](C)(N(C)C)C1] | +++ | +++ | +++ |
| 202 | 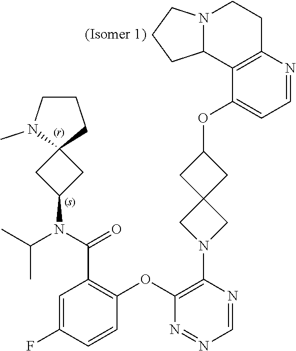 | +++ | +++ | ++ |
| 203 | 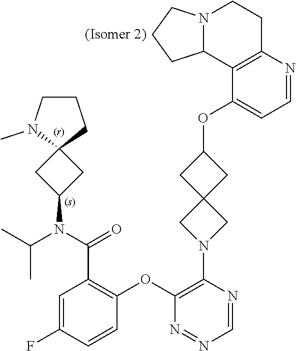 | +++ | +++ | +++ |
| 204 | 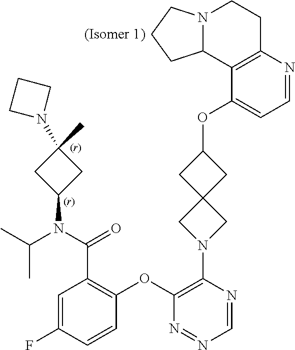 | +++ | +++ | +++ |
| 205 | 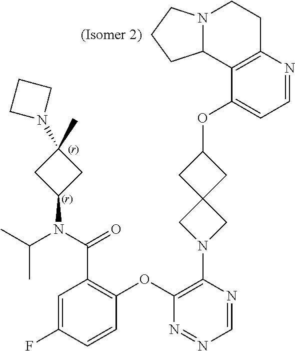 [SMILES: CC(C)N(C(=O)c1cc(F)ccc1Oc1nncnc1N1CC2(CC(Oc3ccnc4c3C3CCCN3CC4)C2)C1)[C@H]1C[C@](C)(N2CCC2)C1 ; "(Isomer 2)"] | +++ | +++ | +++ |
| 206 | 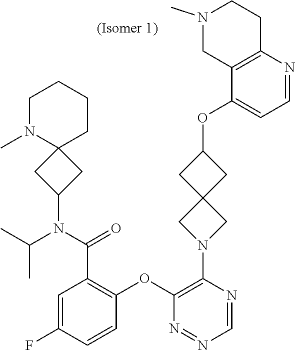 [SMILES: CC(C)N(C(=O)c1cc(F)ccc1Oc1nncnc1N1CC2(CC(Oc3ccnc4c3CN(C)CC4)C2)C1)C1CC2(CCCCN2C)C1 ; "(Isomer 1)"] | +++ | +++ | +++ |
| 207 | 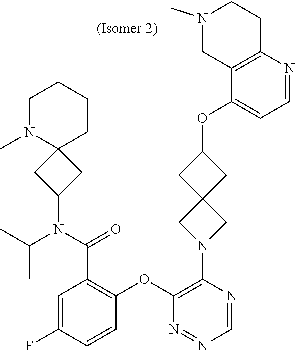 [SMILES: CC(C)N(C(=O)c1cc(F)ccc1Oc1nncnc1N1CC2(CC(Oc3ccnc4c3CN(C)CC4)C2)C1)C1CC2(CCCCN2C)C1 ; "(Isomer 2)"] | +++ | +++ | +++ |
| 208 | 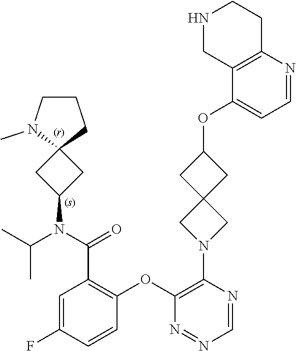 | +++ | +++ | +++ |
| 209 | 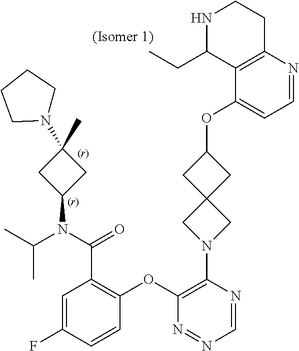 | +++ | +++ | +++ |
| 210 | 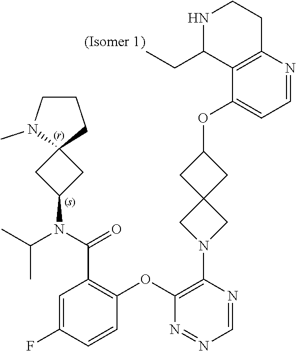 | +++ | +++ | +++ |
| 211 | 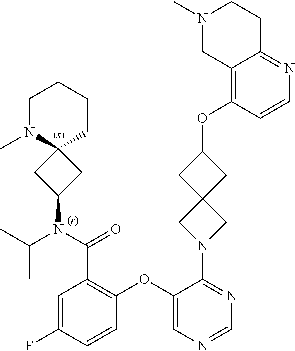 [SMILES: CC(C)N(C(=O)c1cc(F)ccc1Oc1cncnc1N1CC2(CC(Oc3ccnc4c3CN(C)CC4)C2)C1)[C@H]1C[C@]2(CCCCN2C)C1] | +++ | +++ | +++ |
| 212 | 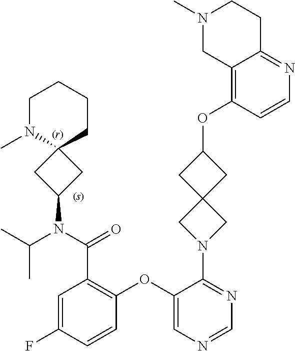 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCCN2C)C1] | +++ | +++ | +++ |
| 213 | 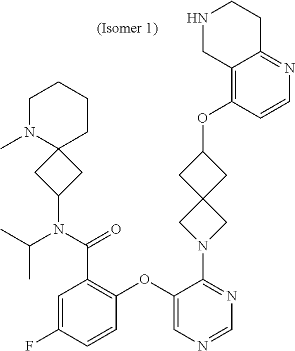 [SMILES: CC(C)N(C(=O)c1cc(F)ccc1Oc1cncnc1N1CC2(CC(Oc3ccnc4c3CNCC4)C2)C1)C1CC2(CCCCN2C)C1 ; "(Isomer 1)"] | +++ | +++ | +++ |
| 214 | 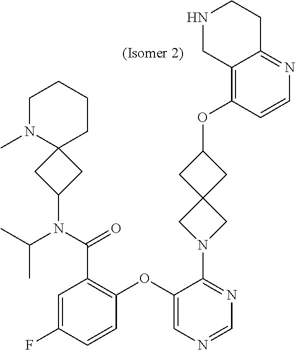 [SMILES: CC(C)N(C(=O)c1cc(F)ccc1Oc1cncnc1N1CC2(CC(Oc3ccnc4c3CNCC4)C2)C1)C1CC2(CCCCN2C)C1] | +++ | +++ | +++ |
| 215 | 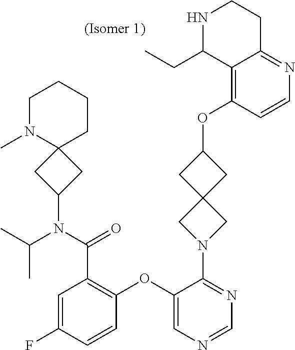 [SMILES: CCC1NCCc2nccc(OC3CC4(C3)CN(c3ncncc3Oc3ccc(F)cc3C(=O)N(C(C)C)C3CC5(CCCCN5C)C3)C4)c21 ; "(Isomer 1)"] | +++ | +++ | +++ |
| 216 | 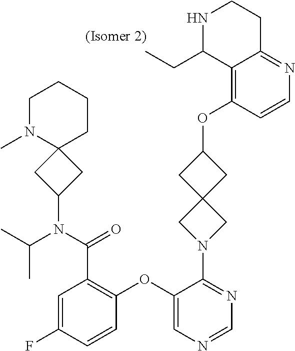 [SMILES: CCC1NCCc2nccc(OC3CC4(C3)CN(c3ncncc3Oc3ccc(F)cc3C(=O)N(C(C)C)C3CC5(CCCCN5C)C3)C4)c21 ; "(Isomer 2)"] | +++ | +++ | +++ |
| 217 | 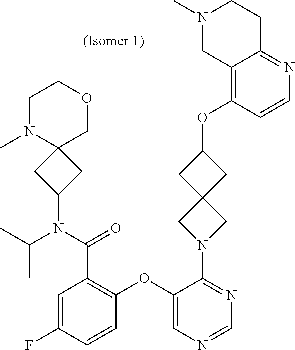 [SMILES: CC(C)N(C(=O)c1cc(F)ccc1Oc1cncnc1N1CC2(CC(Oc3ccnc4c3CN(C)CC4)C2)C1)C1CC2(COCCN2C)C1] | +++ | +++ | +++ |
| 218 | 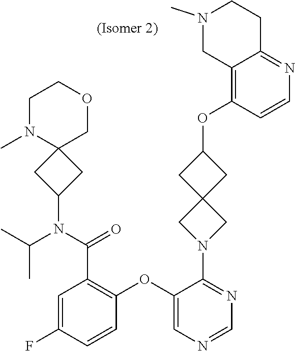 [SMILES: CC(C)N(C(=O)c1cc(F)ccc1Oc1cncnc1N1CC2(CC(Oc3ccnc4c3CN(C)CC4)C2)C1)C1CC2(COCCN2C)C1 ; "(Isomer 2)"] | +++ | +++ | +++ |
| 219 | 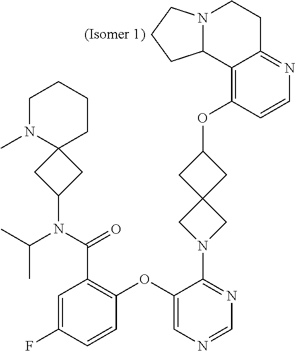 [SMILES: CC(C)N(C(=O)c1cc(F)ccc1Oc1cncnc1N1CC2(CC(Oc3ccnc4c3C3CCCN3CC4)C2)C1)C1CC2(CCCCN2C)C1] | +++ | +++ | +++ |
| 220 | 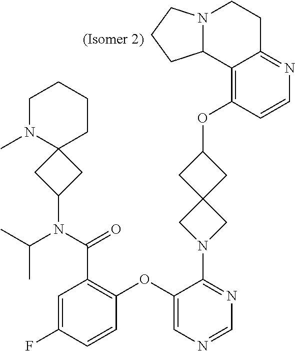 [SMILES: CC(C)N(C(=O)c1cc(F)ccc1Oc1cncnc1N1CC2(CC(Oc3ccnc4c3C3CCCN3CC4)C2)C1)C1CC2(CCCCN2C)C1 ; "(Isomer 2)"] | +++ | +++ | +++ |
| 221 | 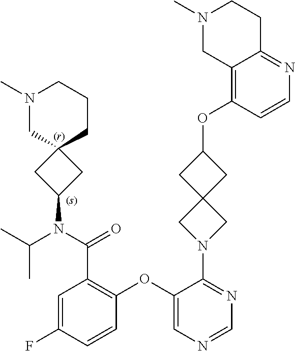 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCN(C)C2)C1] | +++ | +++ | +++ |
| 222 | 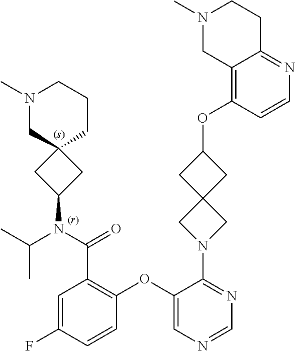 [SMILES: CC(C)N(C(=O)c1cc(F)ccc1Oc1cncnc1N1CC2(CC(Oc3ccnc4c3CN(C)CC4)C2)C1)[C@H]1C[C@]2(CCCN(C)C2)C1] | +++ | +++ | +++ |
| 223 | 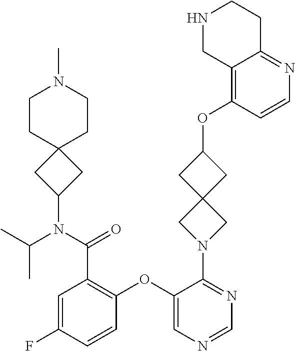 [SMILES: CC(C)N(C(=O)c1cc(F)ccc1Oc1cncnc1N1CC2(CC(Oc3ccnc4c3CNCC4)C2)C1)C1CC2(CCN(C)CC2)C1] | +++ | +++ | +++ |
| 224 | 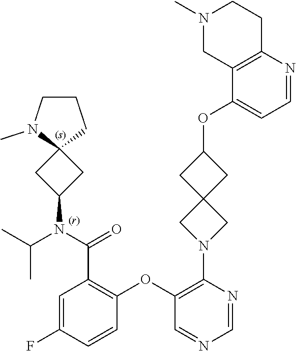 [SMILES: CC(C)N(C(=O)c1cc(F)ccc1Oc1cncnc1N1CC2(CC(Oc3ccnc4c3CN(C)CC4)C2)C1)[C@H]1C[C@]2(CCCN2C)C1] | +++ | +++ | +++ |
| 225 | 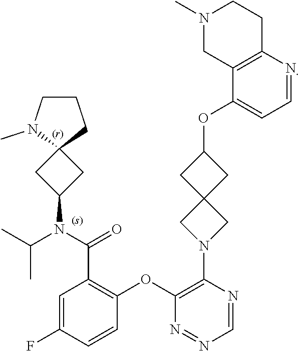 | +++ | +++ | +++ |
| 226 | 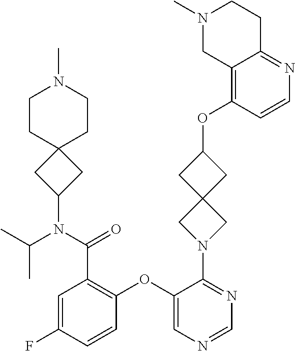 [SMILES: CC(C)N(C(=O)c1cc(F)ccc1Oc1cncnc1N1CC2(CC(Oc3ccnc4c3CN(C)CC4)C2)C1)C1CC2(CCN(C)CC2)C1] | +++ | +++ | +++ |
| 227 | 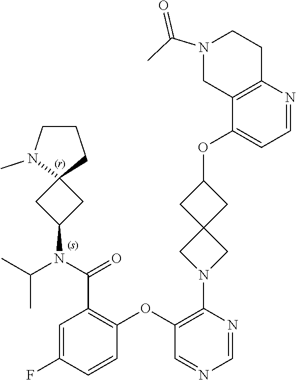 | +++ | + | + |
| 228 | 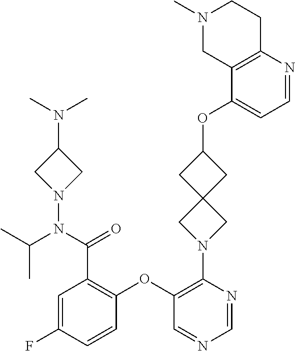 [SMILES: CC(C)N(C(=O)c1cc(F)ccc1Oc1cncnc1N1CC2(CC(Oc3ccnc4c3CN(C)CC4)C2)C1)N1CC(N(C)C)C1] | +++ | +++ | +++ |
| 229 | 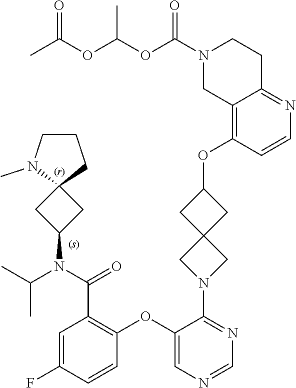 | +++ | +++ | +++ |
| 230 | 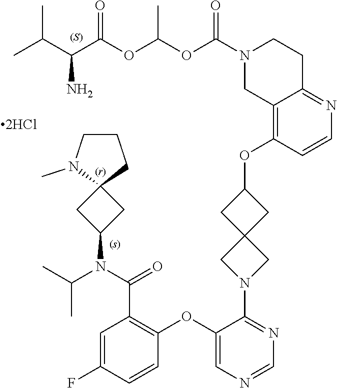 | +++ | +++ | +++ |
| 231 | 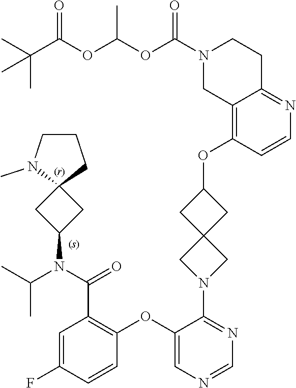 | +++ | +++ | +++ |
| 232 | 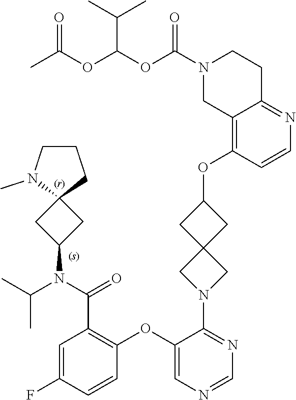 | +++ | +++ | +++ |
| 233 | 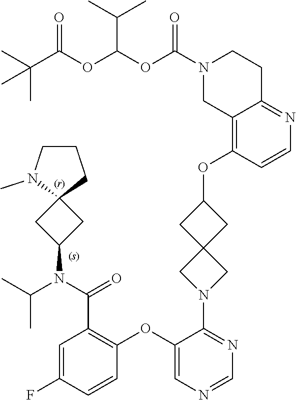 | +++ | +++ | +++ |
| 234 | 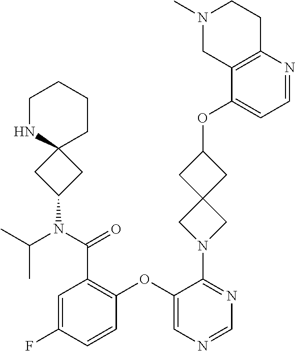 [SMILES: CC(C)N(C(=O)c1cc(F)ccc1Oc1cncnc1N1CC2(CC(Oc3ccnc4c3CN(C)CC4)C2)C1)[C@H]1C[C@@]2(CCCCN2)C1] | +++ | +++ | +++ |
| 235 | 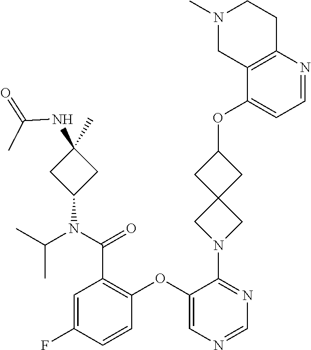 [SMILES: CC(=O)N[C@]1(C)C[C@@H](N(C(=O)c2cc(F)ccc2Oc2cncnc2N2CC3(CC(Oc4ccnc5c4CN(C)CC5)C3)C2)C(C)C)C1] | +++ | +++ | +++ |
| 236 | 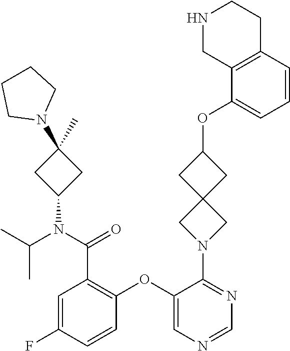 [SMILES: CC(C)N(C(=O)c1cc(F)ccc1Oc1cncnc1N1CC2(CC(Oc3cccc4c3CNCC4)C2)C1)[C@H]1C[C@](C)(N2CCCC2)C1] | +++ | +++ | +++ |
| 237 | 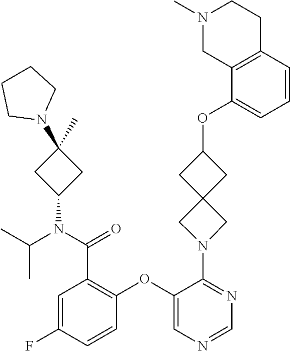 [SMILES: CC(C)N(C(=O)c1cc(F)ccc1Oc1cncnc1N1CC2(CC(Oc3cccc4c3CN(C)CC4)C2)C1)[C@H]1C[C@](C)(N2CCCC2)C1] | +++ | +++ | +++ |

### Example 241. Patch Clamp Assay

The 35 mm culture dishes upon which cells were seeded at a density allowing single cells to be recorded were placed on the dish holder of the microscope and continuously perfused (at approximately 1 mL/min) with the bath solution. All solutions applied to cells including the pipette solution were maintained at room temperature (19° C. to 30° C.). After formation of a Gigaohm seal between the patch electrodes and individual hERG stably transfected HEK 293 cells (pipette resistance range: 2.0 MΩ to 7.0 MΩ; seal resistance range: >1 GΩ) the cell membrane across the pipette tip was ruptured to assure electrical access to the cell interior (whole-cell patch-configuration). In case the quality of the seal was poor, the process of seal formation was repeated with a different cell and anew pipette. As soon as astable seal could be established, hERG outward tail currents were measured upon depolarization of the cell membrane to +20 mV for 2s (activation of channels) from a holding potential of −80 mV and upon subsequent repolarization to −40 mV for 3 s. This voltage protocol (as shown below) was run at least 10 times at intervals of 10 s. If current density was judged to be too low for measurement, another cell was recorded.

Once control recordings have been accomplished, cells were continuously perfused with a bath solution containing the test item 0.3% DMSO or 100 nM E-4031. During wash-in of the test item the voltage protocol indicated above was run continuously again at 10 s intervals until the steady-state level of block was reached.

### EQUIVALENTS

While we have described a number of embodiments of this disclosure, it is apparent that our basic examples may be altered to provide other embodiments that utilize the compounds and methods of this disclosure. The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art. The foregoing description has been presented only for the purposes of illustration and is not intended to limit the disclosure to the precise form disclosed, but by the claims appended hereto. The references cited herein are not admitted to be prior art to the application.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

What is claimed is:

1. A compound of Formula I′, 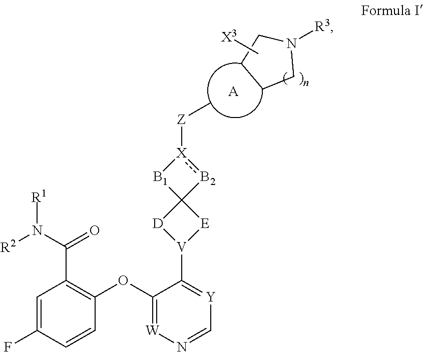
stereoisomer, or a pharmaceutically acceptable salt thereof, wherein: [symbol]
denotes a single bond or a double bond, as valency permits;
B₁, B₂, D, and E are each independently selected from —C(R^{A1})(R^{A2})—,—C(R^{A1})(R^{A2})—C(R^{A1})(R^{A2})—, —C(R^{A1})(R^{A2})—O—, —C(R^{A1})(R^{A2})—NR^{A3}—, —C(═O)—,—C(R^{A1})(R^{A2})—C(═O)—, and —N═C(NH₂)—wherein no more than one of B₁, B₂, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂)—;
V is N or CR^{W}, wherein R^{W} is H, halo, CN, OH, C₁-C₄ alkyl, C₁-C₄ alkoxy, amino, C₁-C₄ alkyl amino, or C₂-C₈ dialkylamino;
X is N or CR^{X}, wherein R^{X} is absent, H, halo, CN, OH, C₁-C₄ alkyl, C₁-C₄ alkoxy, amino, C₁-C₄ alkyl amino, or C₂-C₈ dialkylamino;
each R^{A1} is independently selected from absent, H, halo, C₁-C₄ alkyl, C₁-C₄ alkoxy, C₁-C₄ haloalkyl, C₁-C₄ haloalkoxy, amino, C₁-C₄ alkylamino, C₂-C₈ dialkylamino, CN, NO₂, and OH;
each R^{A2} is independently selected from H, halo, C₁-C₄ alkyl, C₁-C₄ alkoxy, C₁-C₄ haloalkyl, C₁-C₄ haloalkoxy, amino, C₁-C₄ alkylamino, C₂-C₈ dialkylamino, CN, NO₂, and OH;
each R^{A3} is independently selected from H, C₁-C₄ alkyl, C₁-C₄ alkoxy, C₁-C₄ haloalkyl, C(O)R^{z}, and C(O)OR^{z}, wherein said C₁-C₄alkyl is optionally substituted by phenyl, C₁-C₄ alkoxy, C₁-C₄ haloalkoxy, CN, NO₂, or OH;
R^{z} is H, C₁-C₄ alkyl, or phenyl;
W is N or CH;
Y is N or CH;
R¹ is
(a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′},
(b) 3- to 12-membered heterocyclyl which has at least one nitrogen and optionally one or more other heteroatom ring members, wherein the 3- to 12-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
(c) C₃-C₁₂ cycloalkyl substituted with one or more 3- to 12-membered heterocyclyl which has at least one nitrogen and optionally one or more other heteroatom ring members;
and wherein each of the C₃-C₁₂ cycloalkyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Z is O, CH₂, or NH;
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′},C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.

2. A compound of Formula I, 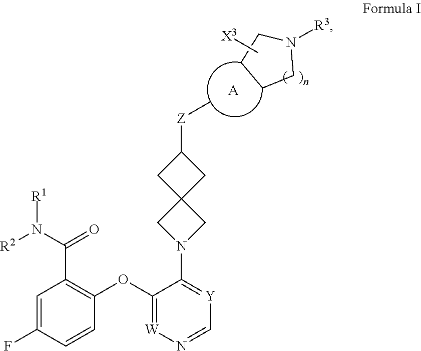
a stereoisomer, or a pharmaceutically acceptable salt thereof, wherein:
W is N or CH;
Y is N or CH;
R¹ is
(a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
(b) 3- to 12-membered heterocyclyl which has at least one nitrogen and optionally one or more other heteroatom ring members, wherein the 3- to 12-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
(c) C₃-C₁₂ cycloalkyl substituted with one or more 3- to 12-membered heterocyclyl which has at least one nitrogen and optionally one or more other heteroatom ring members;
and wherein each of the C₃-C₁₂ cycloalkyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Z is O, CH₂, or NH;
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR^{4a}, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′},C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.

3. A compound of Formula I-a, 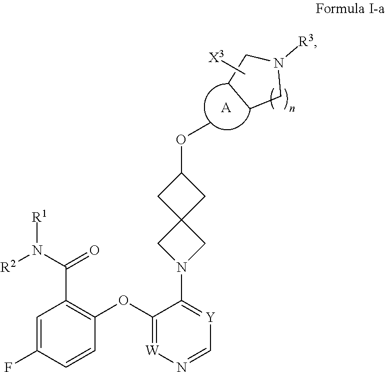
a stereoisomer, or a pharmaceutically acceptable salt thereof, wherein:
W is N or CH;
Y is N or CH;
R¹ is
(a) —C₁-C₆ alkylene-N(R^{3a′})(R^{3b′}) or —C₃-C₁₂ cycloalkylene-N(R^{3a′})(R^{3b′}), wherein the C₁-C₆ alkylene or C₃-C₁₂ cycloalkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a′};
(b) 3- to 12-membered heterocyclyl which has at least one nitrogen and optionally one or more other heteroatom ring members, wherein the 3- to 12-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}; or
(c) C₃-C₁₂ cycloalkyl substituted with one or more 3- to 12-membered heterocyclyl which has at least one nitrogen and optionally one or more other heteroatom ring members;
and wherein each of the C₃-C₁₂ cycloalkyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy;
R² is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl, wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more halo, OH, oxo, CN, or N(R^{4a′})(R^{4b′});
Ring A is 6- to 10-membered aryl or 6- to 10-membered heteroaryl, wherein the 6- to 10-membered aryl or 6- to 10-membered heteroaryl is optionally substituted with one or more C₁-C₆ alkyl, halo, OH, CN, or C₁-C₆ alkoxy;
X³ is H or C₁-C₆ alkyl, wherein the C₁-C₆ alkyl is optionally substituted with one or more halo, OR⁴, oxo, CN, C(═O)N(R^{4a})(R^{4b}), or N(R^{4a})(R^{4b});
R³ is H, C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, 3- to 12-membered heterocyclyl, C(O)(R^{4a}), or C(O)O—C(R^{4a})₂—OC(O)(R^{4a}), wherein the C₁-C₆ alkyl, C₂-C₆ alkenyl, C₂-C₆ alkynyl, C₁-C₆ alkoxy, C₃-C₁₂ cycloalkyl, C₆-C₁₀ aryl, 5- to 10-membered heteroaryl, or 3- to 12-membered heterocyclyl is optionally substituted with one or more R^{3a}; or R³ forms a 3- to 12-membered heterocyclyl with the carbon atom next to the nitrogen atom to which it is connected;
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4b′}, N(R^{4a})(R^{4b}), C₁-C₆ alkyl, C₃-C₁₂ cycloalkyl, C₁-C₆ alkoxy, C₆-C₁₀ aryl, 3- to 12-membered heterocyclyl, or 5- to 10-membered heteroaryl, wherein the C₁-C₆ alkyl, C₃-C₁₂ cycloalkyl, C₆-C₁₀ aryl, 3- to 12-membered heterocyclyl, or 5- to 10-membered heteroaryl is optionally substituted with one or more substituent independently selected from halo, O(R^{4a′}), oxo, CN, C(O)OR^{4a′}, OC(O)N(R^{4a′})(R^{4b′}), N(R^{4a′})(R^{4b′}), N(R^{4a′})C(O)(R^{4b′}), N(R^{4a′})C(O)O(R^{4b′}), C₁-C₆ alkyl that is optionally substituted with O(R^{4a}), and 3- to 12-membered heterocyclyl that is optionally substituted with oxo;
each R^{4a} is independently H, CN, or C₁-C₆ alkyl optionally substituted with C₆-C₁₀ aryl or N(R^{4a′})(R^{4b′});
each R^{4b} is independently H, S(═O)₂R^{4b′}, C(O)OR^{4b′}, C(O)R^{4b′}, C(O)NR^{4a′}R^{4b′},C(O)CH₂—N(R^{4a′})(R^{4b′}), C₁-C₆ alkyl, C₃-C₁₂ cycloalkyl, C₆₋₁₀ aryl, 5- to 10-membered heteroaryl, or 3- to 12-membered heterocyclyl, wherein the C₁-C₆ alkyl is optionally substituted with C₁-C₆ alkoxy or C(O)N(R^{4a′})(R^{4b′});
each R^{3a′} is independently H, C₁-C₆ alkyl, C₁-C₆ haloalkyl, C(O)R^{3c′}, C₃-C₁₂ cycloalkyl, or 3- to 12-membered heterocyclyl, wherein the alkyl, haloalkyl, cycloalkyl or heterocyclyl are optionally substituted with C₁-C₆ alkoxy;
each R^{3b′} is independently H, C₁-C₆ alkyl, C₁-C₆ haloalkyl, C₃-C₁₂ cycloalkyl, or 3- to 12-membered heterocyclyl;
each R^{3c′} is independently H, C₁-C₆ alkyl, C₁-C₆ haloalkyl, C₃-C₁₂ cycloalkyl, or 3- to 12-membered heterocyclyl;
each R^{4a′} and R^{4b′} is independently H, C₁-C₆ alkyl, C₃-C₁₂ cycloalkyl, or 3- to 12-membered heterocyclyl; and
n is 1, 2, or 3.

4. The compound of any one of the preceding claims, wherein W is N.

5. The compound of any one of the preceding claims, wherein W is CH.

6. The compound of any one of the preceding claims, wherein Y is N.

7. The compound of any one of the preceding claims, wherein Y is CH.

8. The compound of any one of the preceding claims, wherein R² is C₁-C₆ alkyl optionally substituted with one or more halo, OH, oxo, CN, or N(R^{4a′})(R^{4b′}).

9. The compound of any one of the preceding claims, wherein R² is isopropyl.

10. The compound of any one of the preceding claims, wherein Z is O.

11. The compound of any one of claims 1-10, wherein R¹ is —C₁-C₆ alkylene-N(R^{3a′})(R^{3b′}) wherein the C₁-C₆ alkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a}.

12. The compound of claim 11, wherein R¹ is

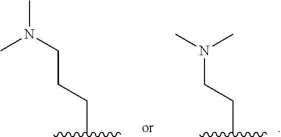

13. The compound of any one of claims 1-10, wherein R¹ is —C₃-C₁₂ cycloalkylene-N(R^{3a′})(R^{3b′}), wherein the C₃-C₁₂ cycloalkylene is optionally substituted with one or more R^{1as};
each R^{1as} is independently halo, oxo, C₁-C₆ alkyl, C₁-C₆ haloalkyl, C(═O)(R^{3a′}), N(R^{3a′})(R^{3b′}), or S(═O)₂R^{3a}.

14. The compound of claim 13, wherein R¹ is

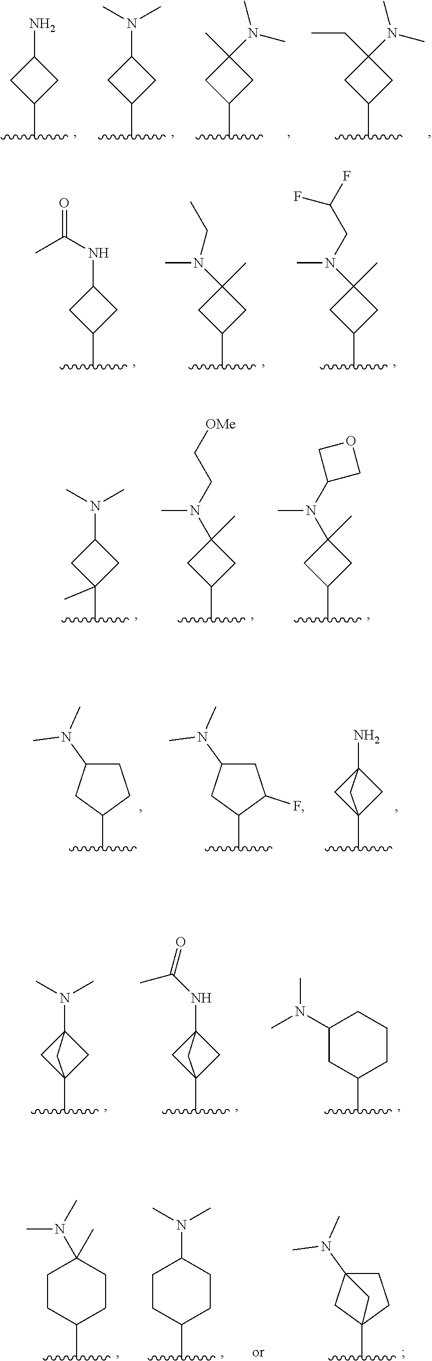

15. The compound of any one of claims 1-10, wherein R¹ is 3- to 12-membered heterocyclyl which has at least one nitrogen and optionally one or more other heteroatom ring members, wherein the 3- to 12-membered heterocyclyl is optionally substituted with one or more R^{1bs};
each R^{1bs} is independently halo, oxo, OR^{3a′}, C₁-C₆ alkyl, C₁-C₆ haloalkyl, C₃-C₁₂ cycloalkyl, 3- to 12-membered heterocyclyl, C(═O)(R^{3a′}), N(R^{3a′})(R^{3b′}), or S(═O)₂R^{3a′}.

16. The compound of claim 15, wherein R¹ is

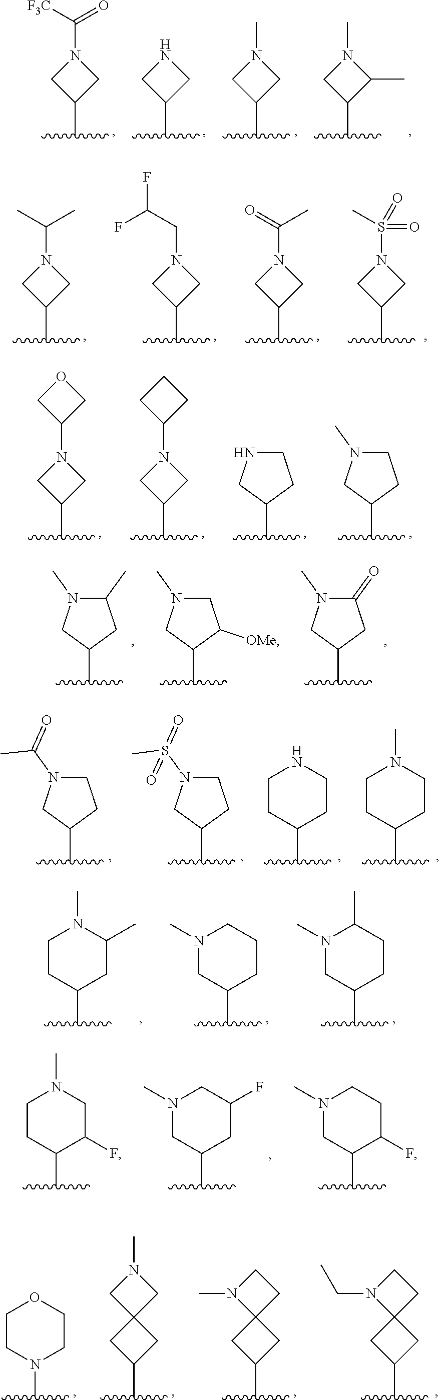

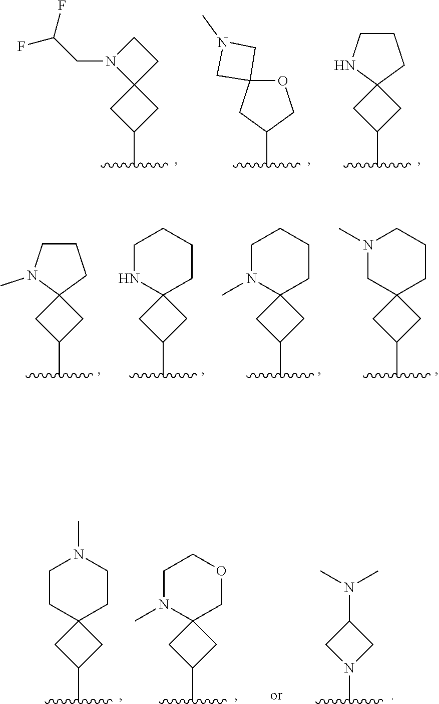

17. The compound of any one of claims 1-10, wherein R¹ is C₃-C₁₂ cycloalkyl substituted with one or more 3- to 12-membered heterocyclyl which has at least one nitrogen and optionally one or more other heteroatom ring members; and wherein each of the C₃-C₁₂ cycloalkyl and the 3- to 12-membered heterocyclyl is optionally substituted with one or more R^{1cs};
each R^{1cs} is independently halo, oxo, C₁-C₆ alkyl, C₁-C₆ haloalkyl, C(═O)(R^{3a′}), N(R^{3a′})(R^{3b′}), OR^{3a′}, or S(═O)₂R^{3a′}, wherein the alkyl or haloalkyl is optionally substituted by one or more C₁-C₆ alkoxy.

18. The compound of claim 17, wherein R¹ is

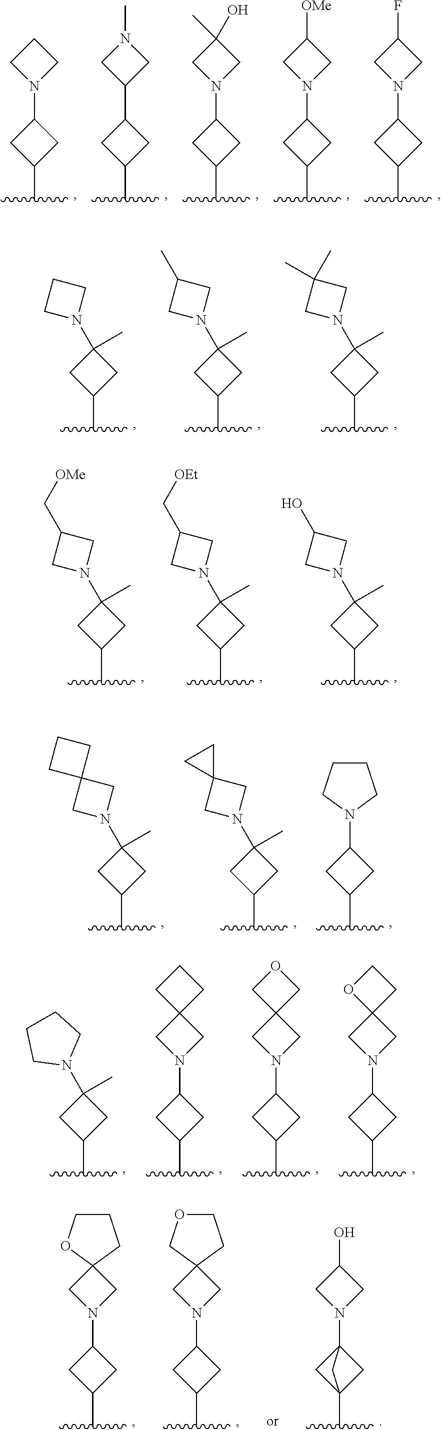

19. The compound of any one of the preceding claims, wherein Ring A is 6- to 10-membered aryl or 6- to 10-membered heteroaryl.

20. The compound of any one of claims 1-19, wherein Ring A is

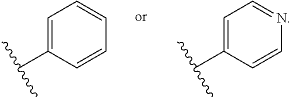 [SMILES: *c1ccccc1 ; "or" ; *c1ccncc1]

21. The compound of any one of claims 1-20, wherein 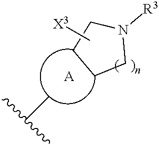
is

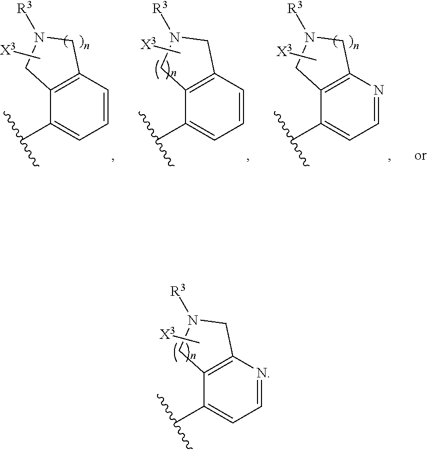

22. The compound of claim 21, wherein 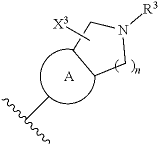
is

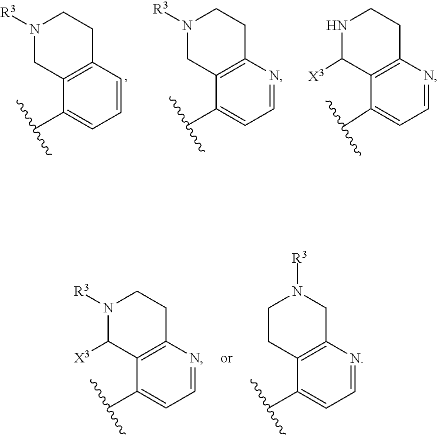

23. The compound of claim 21, wherein 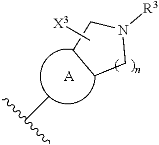
is

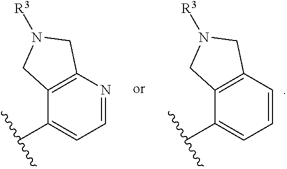

24. The compound of claim 21, wherein 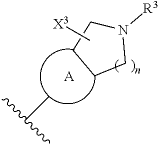
is

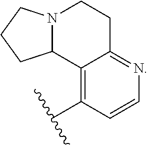 [SMILES: *c1ccnc2c1C1CCCN1CC2]

25. The compound of any one of claims 1-22, wherein X³ is H or C₁-C₆ alkyl.

26. The compound of any one of the preceding claims, wherein R³ is C₁-C₆ alkyl optionally substituted with C₆-C₁₀ aryl, 3- to 12-membered heterocyclyl, or OR^{4a}, and R^{4a} is H, CN, or C₁-C₆ alkyl.

27. The compound of any one of the preceding claims, wherein R³ is methyl, ethyl, isopropyl or tert-butyl.

28. The compound of any one of claims 1-25, wherein R³ is

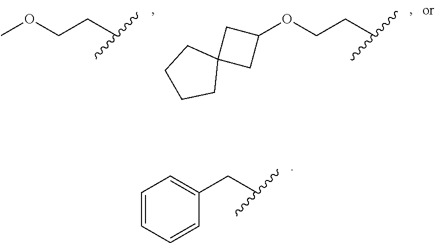

29. The compound of any one of claims 1-25, wherein R³ is C(O)(R^{4a}) or C(O)O—C(R^{4a})₂—OC(O)(R^{4a}), wherein:
each R^{4a} is independently H, CN, or C₁-C₆ alkyl optionally substituted with C₆-C₁₀ aryl or N(R^{4a′})(R^{4b′}); and
each R^{4a′} and R^{4b′} is independently H, C₁-C₆ alkyl, C₃-C₁₂ cycloalkyl, or 3- to 12-membered heterocyclyl.

30. The compound of any one of claims 1-25 and 29, wherein R³ is

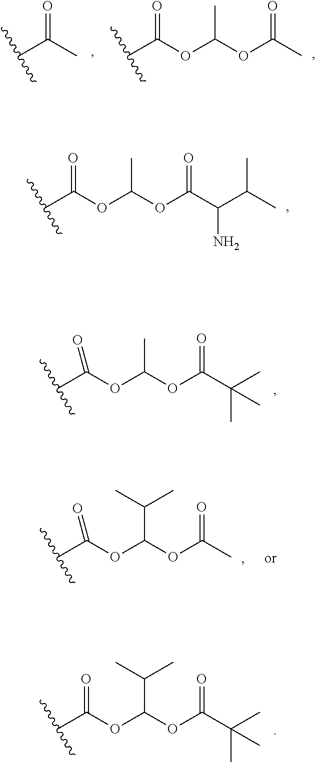

31. The compound of any one of claims 1-25, wherein R³ is H.

32. The compound of any one of the preceding claims, wherein n is 1.

33. The compound of any one of claims 1-31, wherein n is 2.

34. A compound as shown in Table 1 or a stereoisomer thereof, or a pharmaceutically acceptable salt thereof.

35. A compound as shown in Table 1 or a pharmaceutically acceptable salt thereof.

36. A compound as shown in Table 1.

37. A compound as shown in Table 1 or a pharmaceutically acceptable salt thereof, wherein the salt is hydrochloride.

38. The compound according to any one of the preceding claims, wherein the compound is useful for the treatment of cancer and wherein the compound minimizes hERG binding.

39. A pharmaceutical composition comprising a compound of any one of the preceding claims, or a pharmaceutically acceptable salt thereof, and at least one pharmaceutically acceptable carrier.

40. A pharmaceutical composition comprising a salt or crystalline form of the compound of any one of claims 1-38, and at least one pharmaceutically acceptable carrier.

41. A method of inhibiting the interaction between menin and MLL comprising contacting the menin and MLL with a compound of any one of claims 1-38 or the pharmaceutical composition of claim 39 or claim 40.

42. A method of treating cancer in a patient comprising administering to the patient a compound of any one of claims 1-38 or the pharmaceutical composition of claim 39 or claim 40.

43. The method of claim 42, wherein the cancer is a hematological cancer.

44. The method of claim 42, wherein the cancer is a leukemia or a lymphoma.

45. The method of claim 42, wherein the cancer is mixed lineage leukemia (MLL), MLL-related leukemia, MLL-associated leukemia, MLL-positive leukemia, MLL-induced leukemia, rearranged mixed lineage (KMT2A-rearranged) leukemia (MLL-r), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL), acute myeloid leukemia (AML), acute granulocytic leukemia, acute nonlymphocytic leukemia, chronic lymphocytic leukemia (CLL), chronic myelogenous leukemia (CML), therapy related leukemia, myelodysplastic syndrome (MDS), myeloproliferative disease (MPD), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sézary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non Hodgkin's lymphoma (malignant lymphoma), or Waldenstrom's macroglobulinemia.

46. The method of claim 42, wherein the cancer is an abstract nucleophosmin (NPM1)-mutated acute myeloid leukemia (i.e., NPM1^{mut} acute myeloid leukemia) or a rearranged mixed lineage (KMT2A-rearranged) leukemia (MLL-r).

47. The compound of any one of claims 1-38 or a pharmaceutically acceptable salt thereof, or the pharmaceutical composition of claim 39 or claim 40, for use in treating or preventing a disease caused by, or associated with, menin expression, activity, and/or function.

48. The compound of any one of claims 1-38 or a pharmaceutically acceptable salt thereof, or the pharmaceutical composition of claim 39 or claim 40, for use in treating or preventing cancer.

49. Use of the compound of any one of claims 1-38 or a pharmaceutically acceptable salt thereof, or the pharmaceutical composition of claim 39 or claim 40, for treating or preventing a disease caused by, or associated with, menin expression, activity, and/or function.

50. Use of the compound of any one of claims 1-38 or a pharmaceutically acceptable salt thereof, or the pharmaceutical composition of claim 39 or claim 40, in the manufacture of a medicament for treating or preventing a disease caused by, or associated with, menin expression, activity, and/or function.

51. Use of a compound of any of claims 1-38 or a pharmaceutically acceptable salt thereof, or the pharmaceutical composition of claim 39 or claim 40, for treating or preventing cancer.

52. Use of the compound of any one of claims 1-38 or a pharmaceutically acceptable salt thereof, or the pharmaceutical composition of claim 39 or claim 40, in the manufacture of a medicament for treating or preventing cancer.

53. A kit comprising the compound of any one of claims 1-38 or a pharmaceutically acceptable salt thereof, or the pharmaceutical composition of claim 39 or claim 40 and instructions for its use.

54. A method of preparing a compound of any one of claims 1-38 or a pharmaceutically acceptable salt thereof, according to a scheme of the present disclosure or synthetic description in the Examples.

55. An intermediate useful in the preparation of a compound of any one of claims 1-38 or a pharmaceutically acceptable salt thereof.
